SEQUENCE LISTING (62 sequences)

SEQ ID NO: 1 - DNA, 573 bp, Glycine max
aagtgttcgggaatgcaagatccgcctcttaaaaccagttcaggaggaaatgaaaatgaaaactctgagaagaagaattggatgagttcagcacagctttggagcactcagaaatccaaatcgaggaatgaggaagatgataggtctgtgccagcgaactcaattaatgggaattcgtgtgtccccgagaaggagggctcgcaagttccgagcttcggtttgatggctcgtgcttctgacttggtcaataccaattccacaagtgttggtggagatactagttctgcctcctctctgctccgtgttgaggtacagagtcagccacagccgccgcagcatatgcagcagaatccgaggaaacaacggcggtgctggtcgccggagcttcaccggcgctttgtggacgcccttcaacaacttgctggagcacaagtggccactcctaaacagattagagaactgatgcacgtggaaggccttacacatgatgaagtgaacagtcatttgcataagtacacgcttcatgtcaggagattcccagcttcttcaatcggccaggttgataatggctca

SEQ ID NO: 2 - DNA, 503 bp, Glycine max
gcaaagtggaaggaatacaacgaacacctttattctggcaaagatgatagtagaacaactccaaaggcaccggctaaaggttcgaagaaagggtgcatgaaagggaagggaggacctcaaaactctcagtgtaactacagaggagttaggcagaggacatgggggaaatgggttggtgagattagggagcccaatagaggaagcaggctttggttgggtaccttctcttctgcccaggaagctgctcttgcctatgatgaagctgctagagctatgtatggtccttgtgcacgcctcaattttcccggcatcacagattatgcttcttttaaggaatcgttgaaggaatctccgatggccgcatcgtcctcttgttcttcggcagaaactgcaacatctgacactactactacatccaactagcagaatgaagcaagagcctaaggatgaggctgtggatcacatggtccccggggctgggaaaattctagatgtcagaccag

SEQ ID NO: 3 - DNA, 502 bp, Glycine max
  misc_feature (431)..(431): n is a, c, g, or t
agaagtaatttgttcatatgaggccatacaacaggaaagaaaaaaccagaatctgtaattggccacctctgcctaacaatcctggaaccttaaaggtgaggtattcaaaatcatcatcaaaggtcctccttaacttcataaaatagtattatcatccagctaaaatatgattttgctgctagtaaattttgggtaattatcacatcatatcatatattttttttttaaaaattatcacatcataaattaataatgatgttgatgtgttgtattataaagctgttatccttggatgcgttgaatttcttttccgggaaattgattgaagaaatacatatttaattaaaaaaaaaaaaaaaaaacgaaacccgcagaggaacaacaacaacgaagtgtttcatcgtacagaggagtacggaagaggaagtggnggaagtacgtgtcggaaattaggttacccaacagccgtcaaaggatttggttggttcctatgacagcgccg

SEQ ID NO: 4 - DNA, 562 bp, Glycine max
cctacacatttctactctttagtttctccaattttctttctcaattctcaacaatggccactacaacacaacttcacttaccccctggattcagattccatccaacagatgaagaactcgtcatccactacctttgtcgcaaatgcgcttcgcaacatatcgcgtttcccataatcgccgaaattgatctgtacaagtacgacccttgggaccttccaggaatggctttgtacggagagaaagagtggtattttttcacgccgagggaccgcaagtacccgaacggttcgcggccgaaccggtccgcgggaaccgggtactggaaggcaaccggagcggataaaccagttggtaaaccgaaaccggttgggatcaagaaagcgttggttttttacgctggaaaagcgcccaagggagagaaaactaactggatcatgcacgagtatcgtcttgcagacgtggatcgttccgttcgcaaaaagaacagcttaaggctggatgactgggtgctgtgccgaatttacaacaagaaaggtgcaattgaaaagcaacaactaccacc

SEQ ID NO: 5 - DNA, 647 bp, Glycine max
ttttttttttttagaaatcaaaaacatgtcattgccgtttatgttaaggtttcacctcatacatctcaaaagggtggcaaagactactactatttgtacttcacaacaattcacacacacagagaaggacttaaaaagaggaaaaattgaagagatataataaaaactttggccttgttttcaaattcaaccaagcaggtccaaagagtctgtgattgaggaatcagatttgacatagggagaaacatcatcatcaatgggactcaagggaggagggtggtatggactgtgtagctgcatgtcacatatcatttggcgaagttctgaggcttcttctctcagctgaacattctcttgaacaactttatcttgtgacgcagacacatggttcagcttgtccattagctggtggttttcattcctgagccaaaccacctgtgaccacagttcatcaaggtgtttctgcttcctcatgcgtgaccggcgcgccgactcgcggttcgatatcatcctcctgtgcttcctctcattgatgaggctttgctgctgctcatctgcttcatcagaggttgagttgctgctgatgcatgaggactgaggggggctgaagtctgcaagcactttaggaggattctgcaaaccataga

SEQ ID NO: 6 - DNA, 492 bp, Glycine max
  misc_feature (473)..(473): n is a, c, g, or t
ttattcgattgagcaaaaaatgggagttccagagagagaccctcttgcacaattgagcttgcctcctggatttagattttatcccactgatgaggagcttttggttcagtacctttgccgcaaggttgctggccattatttctctcttccaatcattgctgaagttgatttgtacaagtttgatccatgggttcttccaggtaaggcagcgtttggagagaaggagtggtacttcttcagtccaagagacaggaagtacccgaatggttcacgaccaaacagagttgcgggttctgggtattggaaagccactggaactgacaaaatcatcaccactgaaggtagaaaagttggcataaaaaaagcacttgttttctacgttggcaaagcacccaaaggctccaaaaccaattggatcatgcacgagtatcgccttctcgactcttcccgcaaacacaacctcggaaccgcanagcttgatgattgggttct

SEQ ID NO: 7 - DNA, 666 bp, Glycine max
ggcacgaggttaattagtaaagtcacacgtgctttctaacggtggcgcaccagaggggtttaggttcaggttcggggaagttcttggcggcggaacaaggcggcgatgaggaaacggaagagagtactgtaggttcggttccgagccatgtggtctctgcagcttcggcccaaaccaataatcagtgggtcccggcaagtggttctccaatgatgtcacgttttatgaatgctccttcctctccaggattgtctcctctgtttggcgttggccacaagagagcccgtgaagacgacacgggtgcgggtgcaagtgtgggtcttggtgttgttgcagcagcttcttcacaaacacaagggaatgaaagtggcggcggcgagaggaggaggagatacagaggagtgaggcagaggccgtgggggaaatgggcggcggagattcgcgatcctcacaaagcggcgagagtgtggcttgggacattcgacactgccgaggctgctgccagagcttacgatgaagccgcattgaggttcagaggtaacagagccaaacttaatttcgccgaaaatgttgtacaggttcaacctcctcctgcgcttccaaatttaccggcaagtgcggttgctggttccggttccggttcggttgtgacaggttttgcgccga

SEQ ID NO: 8 - DNA, 748 bp, Glycine max
  misc_feature (697)..(697): n is a, c, g, or t
ccatattcaccggcgcccccaagttcgaggactttctgggcggttcctccgcaacagccaccgccaccacgtgtgcaccgccacagcttccgcagttctccaccgacaacaacaaccacctgtacgattcggagctgaagacaacaatagccgcgtgcttccctcgcgcctttgtcgccgaaccaaccaccgaacctcagaaaccctctccaaagaaaaccgtcgacaccttcggccaacgcacctccatctaccgcggcgtcacccgacatagatggacgggaagatacgaagctcatctatgggacaatagttgtagaagagaaggccaaagcaggaaaggaagacaagtttacctgggtggttatgacaaggaagataaggcagccagggcttacgatctcgcagctctcaagtactggggtccaactaccaccaccaactttcccatttccaactatgagaaggaactggaggagatgaagaacatgaccaggcaagagtttgttgcttctctacgaaggaagagcagtggtttctctaggggggcctctatatacagaggagtgacgagacaccaccagcatggccgatggcaggcgagaataggcagagttgccggaaacaaagacctctaccttggaactttcagcacccaagaagaagctgctgaggcctatgacattgctgctatcanattcaggggattaaatgcagtcancaactttgacatgagtcgctacgatgt

SEQ ID NO: 9 - DNA, 655 bp, Glycine max
aatttcatttcatttttccttttcacttcactttcccccaattttctttctcaacaatggccgcagcaacacaactccacttaccccctggattccgattccaccccaccgatgaagaactcgtcgttcactatctctgccgcaaatgcgcttcgcaagaaatcgcagttcttataatcgccgaaatcgatctctacaagtacgacccatgggaccttccaggaatggctttgtacggagagaaagagtggtattttttcacgccgagggaccgcaagtacccgaacggttcgcggccgaaccggtccgcggggaccggatactggaaggcaaccggagcggataaaccggttggcaaaccgaaacctgttgggatcaagaaagcattggttttttacgccggaaaagcgcccaagggagtgaaaactaactggatcatgcacgagtatcgtctcgccgacgtggatcgttcggttcgcaaaaagaacagcttaaggctggatgactgggtgctgtgtcgaatttacaacacgaaaggtgcaattgagaagcagcaaccagcaccgccaccaccgagtggggtccacacaattgaatgttacgaaatggaagacgcgaagccggagtatccggcggcggattgcctgtacttcga

SEQ ID NO: 10 - DNA, 708 bp, Glycine max
gagactcatcttcagcgacagaagaagcccccacctccacaactacaagtgtaaccgctgtggcaacaacggttactgaaaccccacccccaccgggaggtggggaaaggaggagaaagtacaggggagtgaggcagaggccatggggaaaatgggcagcagaaattcgtgatccacacaaagcagcaagagtttggcttggaacattcgacacagaagaagcagcagcaagagcatatgatgaagctgcattgaggttcagaggcaacagagcaaagcttaacttccctgaaaatgtaagagcagttccacccattcaaccttttcaagccaccactaggctaaccgtttctgattccaccacctctcaattccggccactcgtcacggagacgccgcccttcattcagcagccacagattcagggctcctctgacttgatcagagattacttgcaatactctcagcttctacagagtgattttcaacagcaacaaatacaacaacaatcaccttctagtttgcttcagcagttgtactataatgcacaattggcttcacttcaatcaccttcagtgctatcatcatctccttcattttcttcttccgtgtcaccaacaccattcccattgttctcaacctctgctcagttttcctctgcttctttccccatgttttcaagtcaacaaatgggctatttccagctgc

SEQ ID NO: 11 - DNA, 536 bp, Glycine max
gccgaacggttcgcggccgaaccggtccgcggggaccggatactggaaggcaaccggagcggataaaccggttggcaaaccgaaacctgttgggatcaagaaagcattggttttttacgccggaaaagcgcccaagggagtgaaaactaactggatcatgcacgagtatcgtctcgccgacgtggatcgttcggttcgcaaaaagaacagcttaagggtatataccagtatgcgcccttttttctcttcactatcgaggacataatagacatttcaacgcacatttacctttcgaaaatcaccgttttaccctttaatgaatcacacctggccccacccccccttggcaagcgcgcagagaagcttcggtttacgataagttgataaggaccactcacagaaaaaagaaaaggaaaataattacaataataaatgttttcgccaaccaattattactttcctacatatggaaattattcgttctttcttatctctattgaagttagggatatgagaaattgaaaattaaaaaaatg

SEQ ID NO: 12 - DNA, 650 bp, Glycine max
  misc_feature (325)..(325): n is a, c, g, or t
acgatgatcacaacaacaaagaaaagaacgggtagaagaagtccaacatcggataagctcaagaatcaacaccgcgagaagcagtcgatgaaaccttaccgtggaataaggatgcggaagtgggggaagtgggtggcggagatcagagaacccaacaaaaggtcgaggatatggttgggttcttacacgacacccgtggccgccgcacgtgcctacgacaccgctgtcttttacctccggggtcccaccgcgcgccttaacttccccgaactcttgttccaggacgacgaccaggagggcagtgattcggtgcagcacggcgcancagggaacatgtccgctgattccattcgccgaaaagccacgcaagtcggcgccagagtcgacgctctccaaaccgcgcttcaccaccacgcgccaagtaccaactctctcaatctcaagcccgacttgaacgagtttccaaaactcgaagaccttcaagattgatataaatcaaatatcaatatcaatcaatatatttaatttcctaagttctttattaatatatagttttatgtgtgtatatatagatgatgatgcctcggagttggggcttgaaactaattaaccccttcccttccccttaatttagatatatcccttttctt

SEQ ID NO: 13 - DNA, 481 bp, Glycine max
gatgatgaagaagaactcttaactgctaataacacctcttccaaagtgaaggtggatgaacccagcacggggaagagggtgaacccacagcgttcaaagcattcagaaaccgagcaacgtagaaggagcaagattaacgaaaggtttcaggttctgagagatctcatacctcaaaatgatcaaaaaagagataaggcatccttcttgttggaggtcattgagtatattcagttcctacaggaaaaattacaaatttatgaacagacttatgaaggatggaatcaggagcccacgaaattaactccatggagctagtagcccagtatgcatatttgaatgagttcaggatggcattgtggcaacttcagtgatctatttgtcacaatgagaaataatcacgggcctgcagaaaatactacagatccttctcaagctactcaaaatgggtctgttgatgagaaaaataacaatgtctctccat

SEQ ID NO: 14 - DNA, 753 bp, Glycine max
  misc_feature (580)..(580): n is a, c, g, or t
caaagtttcatcaatcatcaccagtggttcttttccttctgagaagatcgaattgtgttttgcaaaaatgggagttccagagaaagaccctctttcccaattgagtttgcctcctggttttcggttttaccccaccgacgaggagcttctcgttcagtatctgtgccgcaaggtcgctggccaccatttctctcttccaatcattgccgaaattgacttgtacaagttcgacccatgggttcttccaagtaaagcgattttcggtgaaaaagagtggtactttttcagccccagagacaggaaatacccgaacgggtctcgacccaacagagtagctgggtcgggttattggaaagccaccggaaccgacaagatcatcaccaccgaaggtagaaaagttggcataaaaaaagccctcgttttctacgttggcaaagcccccaagggcaccaaaaccaattggatcatgcacgagtatcgcctcctcgactcttcccgaaagaacactggcaccaagcttgatgattgggttctgtgtcgtatatacaagaagaactcgagtgcacagaagacggcgcanaacggcgtggttccgagcaacgagcacactcaatacagcaacggttcctcttcttcttcttcgtcccagctggaggacgttctggaatctctgccatcgattgatgaaaggtgtttcgcgatgccacgcgtcaacacgctgcaacaancacagcaccacgaggagaaggtcaa

SEQ ID NO: 15 - DNA, 556 bp, Glycine max
caatttgccttcacttcataataataataactcctcttttcctgtaccaaaccaaattgatattttggactcaatggagggtggcacaacaagctctgaactcccccctggctttaggtttcatccaacggatgaggaactgattgtgtattacctttgtaaccaagccacctcaaagccatgccctgcatctatcatcccagaagtggatctctataagtttgatccatgggaattgccagataaaacagaatttggagaaaatgaatggtatttctttactccaagagacaggaagtacccaaatggggtgaggcctaaccgagcaaccgtgtcagggtattggaaggctactggcacagacaaggcaatctacagtggatctaagcatgtaggggtgaagaaatctttagtcttttacaagggtaggccaccaaagggtgccaagacagattggatcatgcatgagtatcgattggctgaatcaaaaataccatctagcaggaaaattggatccatgagggatctataagaagaagagcatggtaaaggcatt

SEQ ID NO: 16 - DNA, 678 bp, Glycine max
tttttttttatcaacaaaaacaaactgtcatagcaaatggcattaacagtaacaactaaaaaaaacaagattcctctccagctataagctgaaatattattaaccaaaaaagaaaacaaattattgaaatatgaataaacaaaaactatcactaactagtatccgattttgaaatttttaactggctccaatattgccaattctaagtttctcagcggatcaagattatgacaattaacgatgatccataatcagaaacatgaaaatagacaaaaacaaaactaccttaattaatttcagtgggtgtcatcgttggtgctgtttgtgttgtaaggatgattattattattatctgtactatcactaccggtgccattgattctttcacctggttccaacgatttcttcctgccagaagtgttctagtagttgtgcatccacaccttgaagacccctcttgaaacgccaatctccttgcagaaatcttgcaccaatccttcatcccctttctgcattctccaacccaatttctcggcgaaatggtgcattttttctttctgttcatggctgaactgggtcctatgcctcttcttcccacccgagtattctggattccagtgggaagaagagttaaaattaaaactcatgtgatgatgatgttcatttccaagggccaag

SEQ ID NO: 17 - DNA, 508 bp, Glycine max
tattttttttttttttttttagggtaaaattctatttgtaatttattcgtctcgaacttggcaatgtcacccctaacatgtataatatatgcatcttttaagttatcaggagatattaaatttaattagcatccagagtgtggtatggttgatcttcatcagaacataaaaaaattctgataataattcataagtaaatcgtaaaaagccaagcactgcatggaacaatcagcaatttactcttgaaccacgcatttacacctccgttggtgctctgacagttcctcaatttgcttttgtagaaacttgacatacgctacagcctcatctaacatatctgcagtatttgtttgcttatccatgtttggcacaagttcttgcagctttctgatgcggtcactgatacgagtcctgcgaaccctttcagcaatactcctgggatgagtagcacagccacgcttagccctcactctgcaaggaacggaatcttctaacatgaagatttcag

SEQ ID NO: 18 - DNA, 592 bp, Glycine max
  misc_feature (527)..(529): n is a, c, g, or t
tcatggttgaaacttcttcaactgtcactagcaaggtaaataaatacgatccaaccaaacaaaataccaatctctaacctctctacagtaccaaatacaaagaaatgctgttcaccataaaaatctcccctaccaaaggtcaatatttgataaaatcacttcaatttacattttacctgcatcatgcgacatactaacgctacctaccacacaacctacagtgttgtacaaagccagaatgaagcaaaagtgggatatccaaggcaggtattattgatcttgtgacatacaatatagatctccactgtctaaggtatcgtcgtctcagtagtttaggatgtcctatctgatgaggtttgatcatttttattagaattaagattgatattggattgtgatttgtcagctgatgtcacatcatcatgctgattctcgtctttctttgaattcagaaaccgaccaccacatcctcttggtcttctcagtgcatgcttgtgtcgagattcatgcaagcatggcttgctatnnnttataacnnnattttctgatnnnncttttgcacggnannnnnnannnnntaatataccgcggc

SEQ ID NO: 19 - DNA, 754 bp, Glycine max
  misc_feature (21)..(22): n is a, c, g, or t
acgttattaactcatataaannatacatgaaatgcctgaacctgatgggttccatataggagctgattacatgcctgacccacaatcaaggacttgcctgatatgttaaacaacaaactttaaattggatataaatagcaggaactccaaactacataaaattggaacttgtgggattgacgtcctccattagctaattccctcaattgggtaattttaaggtattgctatccactattcagttaaaggtggaagaacctgctttgccactaacagcatcatcaatattagctgttccactcatgggtccgctgctattattgcctggtgcaatcattgtttgactatgttgcactgcttgggaaccatatctgaacacattcataggctgagaggcacttcgcataaaatggtaacccatgtaaggtgcatattgttctggaaggcaagggttttgcatgtgggtttggtaattaaaggcacccaaaacttgattttggcacattaaattctggtttggcatctgnngcacagacatggcttgatcatgtggcacttttggcaattgcatcgggttgtaaatggannncaagnnnnntgcacccattcccatacccatttgtgacatatagtgctgaattcntnnnaacatcactggtgtcatgccnnnncccatccacattacctgaagctgaaactgaagcgatntcaagtattcgnnnnnntcttctaacattgatgctttatctnnctt

SEQ ID NO: 20 - DNA, 586 bp, Glycine max
aagggaaagaatgcagaagtgagtcaaaggataacgtttagaaccagatcgcaacttgaggttatggatgatggatataaatggaggaaatacgggaagaaaacagtgaagagcagtcccaacccacggaactactacaagtgttcaggtgaaggatgcgatgtgaagaaaagggtggaaagggatagggatgacttttctatgtgttaacaacgtacgacggtgtccacaatcatcagaccccgtctactgcctactacagccaaatgcccttgctgcattctaatcatgattgggccctccacccttctgcaaactcataatatcctccttaatatagtaatatccagagataggagaatcaacttatacttttaggattactttttatctttattatttattttttataagagattttaaaaaataaataatgagaatagagaataattttaaaagaaatattgttgatgtaagttaatctctactctgtatatatatatagttattgactcatttagtatattgcctgtcaataaccattattacatgtatattgattattatgcatattccagtccacgct

SEQ ID NO: 21 - DNA, 618 bp, Glycine max
  misc_feature (7)..(9): n is a, c, g, or t
tttacgnnncaccagaatcgccgctggaagcggnagtaaaatgcgactacataaaattttgtaccaacaactctacactcttccaaagctccaacgagtacgacaaaatttcaaacttggacaacgagagggggaggggggcaatnnngggatttccaactcacacttccgcaatgcaaacagggtgtaatttaccaaactgtccctgagatttttatcactagtaaggccacgtcatgaactcgtccagatcgaattcatcaaacgcctcctcctgcttcttcccgtgatgacacgtcagcgcaacgttaacgttaacttccggcaaacttaacggcgcgtcgatgtggaacccgaacgcgtcaacgtcgagataacggaaaccgtcgaacggcgtcgagtcgcactcgtaggccaagacggacgtcggcgaggccactaggtccgagtacgacgcgccgccgtcggagctgaggttgtccgcggcgagcggtggagactgcgccgtnnnctccggcgcgagggggaagttggtgacggcgttggggcccttgagtttgacggcggctctgtcgtactccgtggcgnnntcnnnngncgtgtcgannnnnccgagcc

SEQ ID NO: 22 - DNA, 558 bp, Glycine max
  misc_feature (4)..(5): n is a, c, g, or t
ttgnnacnnttaacaagtaagtaagtaagtaagtaagtaagtaagaaattatattagagaagaaaagaaagaaagaaagaaagagagagatttcagtaatatgatttcatttcatgtgcaaggttctgtaatccaaaacgacggtggttttcaaggccaagaaatcatagccagtagcagtagcgttggcagtactagtgtggagggcacagctacaatctttgctattatgcaaacccagactgcagggaaagcaaaggtttgagtttgtcacaagggtcttcaagtgcggatgatcaggttgaggatgatggggaggactaatggttagctcaaggttcaagtcagggcagcgttcatggtgaaaggactcttgatgtttaggaggggcagagacagcnnnaggttcttgatgagaagaatcgttgagaggcctgtgagtggcagggtcaatacctctgctcnnanncttcctgcgtatgtgagtattccagtaattcttgatctcattgtcagttctaccgngcannntaccngcaattagannccacttgnngc

SEQ ID NO: 23 - DNA, 545 bp, Glycine max
cagattctcaacataccttacaatcagacccttaaaccatcatcttgaaaaatagttctaaatttcccaccatggttcctagtgagatcagaggagtccactatctagcacctgaaaatccattcctagttccaccaaactttagccttctgcaaagtgacatccctaatctccatcttaacaccctcctaagcaactttccaaactgccacttccctccttcaggccatgaatttgttgttcctccctcttcatgcctcagcagcaactcaacctctgatgaagctgatgaaatccagttcaacatcatcgacgaaaggaagcaccggagaatgatatcgaaccgagaatccgctcgcaggtcaaggatgaggaagcagaagcacctggatgagctgtggtcacaggttgttaggctcagaactgagaaccacaaccttattgacaaactcaaccatgtgtctgaatcccatgacagagtgctccaagagaatgcacgcctcaatgaagaagctttcgctcttcgccaaatgctagcagacatg

SEQ ID NO: 24 - DNA, 377 bp, Glycine max
  misc_feature (219)..(219): n is a, c, g, or t
gaagcttcaatgctggatgaggcaattgagtatcttaaaacacttcagctccaagttcaaattatgtcaatgggagctggtttatatatgcctccaatgatgttacctgcaggaatgcagcacatgcatgcaccacatatggctccattttcacctatgggtgttggcatgcatatgggatatggtatgggttatgggatgggaatgcctgacatgaanggtggatcttcaagattncctatgattcaggtgccccaaatgcaaggaactcatattcctgttgcacatatgtctggatctacatctattcatgggatggccatagctaatcctccaatgtttgggcttcctggacaaggacatcccatgccaatgcc

SEQ ID NO: 25 - DNA, 536 bp, Glycine max
tgaagaccaattggatcatgcacgaatatcgcttagccaacgtcgacagatccgcctcaaaaaaaaacaccaccaccaacaacttgaggcttgatgattgggttttgtgccgaatttacaacaagaaagggaagattgagaagtacaatggtgttgcggtggtggatcagaaagtggccaagttatcagaagaggaggttcagttccagcacgagaccaagcctcagatcaagatgtacggccacgatgatttcaggaacgaccaattgtacatggacacgtcggattcggggcccaggttgcacacggactccagctgctccgagcacgtggtttcgcccgacggcacgtgcgagaaggaggtgcagagcgagcccaagtggaatgatcttgagttgggccccgacccagccttgggcttcgattttaatttcatggacctgacctcagatgacgctattgcccctcaggttcaataccaaatcaaccatctctcaccgtggcaagaaatgttcacgtacctaccaaagacattt

SEQ ID NO: 26 - DNA, 554 bp, Glycine max
gagtaaagcaccctcttcaacctcggtagccgccgcgagttctagcccgcagaacccgcagcagaaaaatctctgaatgagggacatagaaagcagcgcaggtgctggtcacaggacttgcacaagcgcttcttgcatgcccttcagcagcttgggggtgcagatactgccacaccaaagcaaattagagagataatgaatgttgatggcctcacaaatgatgaagtcaaaagccatttgcagaaataccgtttgcacactagaaggccaagtcccatggttcacaacagttccaatccacaagcagcaccttttgtccttgtaggaaacatttttgtccaatcaccagaatatgcagctgtagctacctcaacagcatcaagagaagtggccacagttgcagcacctgctggaatttatgcaccagtggccacacaccctatacctgtttcacacccaccagctgattcaattaagaatccacagtttaagaaggtacagctgtttgagcattcaatttcagatgaaagggctaatcacagtgaaggagctgt

SEQ ID NO: 27 - DNA, 476 bp, Glycine max
tggatgatggctacaagtggaggaagtacggacagaaagtggtaaagaacacacaacacccgagaagctactaccgttgcacacaagataactgtcgagtaaagaaacgcgtggagcgcttggcagaggatccaaggatggtgataaccacatatgaagggagacacgtgcactcgccatcaaatgaacttgaatactcgcaaaccccttctgaacttagtaatttcttgtggtagtttacaccaacataatgtttacatatatgtatattttaatcgtagagagtagagatcgatgaagaccgaataagtagagaggaatgagtaaaaattgacagtgtatcttggaaatgatgcatgcatgctttgtttggttcattgtcaattttcactcgtttccatccattcctcccattttcaccctttccaagagcacaataaatatacatatacagttgtgctttttttaaaaaaaaa

SEQ ID NO: 28 - DNA, 545 bp, Glycine max
cgaccatggggacgctacgctgcagaaataagagatccgtggaagaagacacgtgtctggctgggcacattcgatactcccgaggaagccgcccttcgcctacgacggcgccgcccgctccctccgcggcgccaaggccaagaccaacttccctccggcgccgcctctctgcctcgacctcaacgtcccttcctccgatcaccgctggccccacgtcccccctcgcagcctcgtcctcagcgagttcctccacaccgccgttctcagagacatcgacttgaacgaacccccacccttgtggctatgaataatgaatgaaccctgactaggtttctttcgacgtcgttttatgttccctagtattatctactactactcccatgttttttcattttaattttccctcagcatgcttaggtttttattagtagcgttgtacgttcatcgtagagggagtagtcgtatgtatctgtccatgtgagtctaacattaggttaatggaaggggaaaaaaactgctatgatattttgtaaaaaaaaaaaaaa

SEQ ID NO: 29 - DNA, 572 bp, Glycine max
gcacgagggaggatcttacattgaaaacttcaaaaaagaccgaacaagaacaaggtgaaaattcccaagataaaacactgagaaagccggacaaaatactgccttgcccccgctgcaatagcatggataccaagttctgctactacaataattacaatgtcaaccaaccccgccatttctgcaagaattgtcagagatactggactgccggtggagtgatgaggaatgttcctgtgggtgctggtcgcaggaagaacaagaattttacttctcagtatcgtcagataactgttcctgaagcagcactcttgagttctcagccggacttgccaaatggagtcagtcatccttccttgaactgtaatgccacactccttgcatttgggtctgatacacccttgtgtgaatcaatgacatctgttttgaaccttgctgaaaaagcagtgaacaattccacaacaaatggatttaatggctcagaagaactagaaattccttttgcttatgttgctggagaaaaagtgaatgatgattccaataaatcttctgacatgtcaacaaagccaatggaa

SEQ ID NO: 30 - DNA, 673 bp, Glycine max
  misc_feature (620)..(621): n is a, c, g, or t
gaatcatgtgacatataggtgatcttttaatctaaaaacagattatgtcgaatctagcgatcatttttcatccattcctctcacctgtacctcaccccctaccaaatcaagcatggcattcaaattgcacaagttaacacaaattattatttcaacttttcctacaaggccaaatccgggcaaaacacacccagaaccatggatcttcttatggatcttccacaagcagtggatcaatagaatactttcaggctgaatcaaattacttggaattatttggctgcacctattttctctatacaacaatgtaactccgagaacctgcaatggcatgcaatctctcaaacctttccctccttatatctacaccaggccacaaagcctagtctgtccattagtgatgacaaacagatcacaatgattgtgctgaatttactcttcgaagttttttcttcggtttaggcatcacctcaattggaatgatgcactcgaaaagctgctttttcctctgccttctcatctcagcctcttcatttaataactgcacattctcttgctctagctggtgaaccagatactcaagctcagatgtgtaagcctgcttgcgttccctggacctnncagcnnnctctctattcttgatcatcctcctctgcttctgaagcgttgcctt

SEQ ID NO: 31 - DNA, 655 bp, Glycine max
  misc_feature (553)..(554): n is a, c, g, or t
aatccatcatgttacaataattttcatgccaaggcctattatatagtatatgtgcaaaaagataacgtgggttttacaagtgtgatcatgatgagaatgaaaactaaaacttaaatatcccagtgcagcaacaaagtcctcatctatattctcctacagaaactaataacaagaaatcacataaaaacagtaatgaagtaatcatctaatcttctttcaacatatgtgaagaaacaacatcttgaagaagcccatagtcacttaacaaggcagtctcttgaggaaaagtggcattccttgaagacgaagaagagaggaaacccaaagaggacaatgcatcgatgtggacttgttgatgctgctgctgataatattgggacatgtagtttccagcagatccagacataattaccgaaccaaagttgttggagcgacccataactgggcttggatgcgtgtgttgtccttcgtaggttgtcaccacaatgcttgggtcgctaaaagatcgctccacacgtttcttcacattacatgaaacactggtgcaacgatnntagctcctgggaaaggggctgtttttcacagctttttgaccgtactttctccatctgtatccatcttccagatgatccacctcgcttttcgtcatgaacgc

SEQ ID NO: 32 - DNA, 433 bp, Glycine max
ggaacagagaatgtaaagaaggaggagggaaggagaaaatatagaggggtgaggcagaggccgtggggaaaatgggcagcggagataagagacccattcaaagcagcaagagtatggttaggaacatttgagacggcagaggcagcagctagagcctacgatgaagctgcccttcgttttaggggcagcaaggccaaactcaacttccccgagaatgtcactctccgacaacctcaattcactgttttcacatcaacagacccatttcaactctcgggtaccaacctttatgatcataggatgtttctgacttctaataccatggcttctcatcatcttcaatcttctttgccgctgccaccagcaacctcctcttctctaacttctcctcctcagaacatcacttcactttcttccttgtattctgcccagt

SEQ ID NO: 33 - DNA, 487 bp, Glycine max
caaatataaaaaggtttttttcttcttcttcaaattagcataggtgaagagggcaagttgaagccctatatatatgtcacatcaggacgcgtgattggtggatcaatattaatcttgaaggtggaagaaaattactaatcttcaggtttgggatactcgttcaagtctggtttgtcagagctgctgacgtggctggaattagcggagtgtgaggacgaggattgctgaagcgcggtttggagcgcgtcgactctggcgccgacttgggtggcttttttgcgaattgaatcggcggacatgttgccctgttgttgggtcgaaacgtcgtcgtcctgggacagcaattcggggaagttgaggcgagcggaagggcctcggaggtggaagacggcggtgtcataggcgcgtgcggcggcgacgggggtggcgtaagaacccagccagatcctcgaacgcttattgggttctctaatctccgccctcgtgc

SEQ ID NO: 34 - DNA, 476 bp, Glycine max
  misc_feature (440)..(440): n is a, c, g, or t
ggccctcgaggccaagaattcaagcgaacaggtaattagttgcagattacggtgggttaattacctccaccccgatctcaaacgagggaagctcacaccccaagaacaacacctcgtcatggaccttcattccaaatggggaaatagatggtcaagaattgctcgcaagctaccagggcgcactgacaatgagattaagaattattggaggactcacatgagaaagaaaaaggctctggagaaaaagcatgcatcagcatcagcatcatcacctgtttcttctagttgtcaatcttcactatcttcaaacaaccatgttgtggattcacatgcttccaaagagagcttttatgacacaggtggatatgatgacatgatgatggggttaattggtggtgaaaaggggtactctatggatgatatatggaaagatattgttntgtcaggagaagagaacagcatttataacaacaata

SEQ ID NO: 35 - DNA, 634 bp, Glycine max
  misc_feature (587)..(590): n is a, c, g, or t
tccaaaaaaagaagacaaatggtgggaaaccaaaaaaaaacgaaacatataataacggttattgaccagcaatatatgaaagaacatatatatgtatatatgtcaaaaacacacacacacgcacacacacacacacacatatatatatatatatatatatatatatatatatatatatatatatatatatatatatattactgtattgaagaggatcatgagtttgcagaagggtgcagctggggccaatcatggttagaatgcaccaagggaatttggctgtagtaggcagtcgacgggctctcatgattgtggacaccgtcgtacgttgttaaaacatagttcgagtcatccctgtctctttccacccttttcttcacattgcatccttcacctgaacacttgtagtagttccttgggttgggattgttcttcactgtcttctttccgtatttcctccatttatatccatcatccataacctcaagctgcgatctggttctaaacgtgatcatttgactcacttcttccttctttcgctttatcccattattttcgcactttatatgcatgttggtgttgctgannnntgcatcaccaaacccatgactgacatcgctggagtttcctttct

SEQ ID NO: 36 - DNA, 654 bp, Glycine max
  misc_feature (493)..(495): n is a, c, g, or t
tgagcactgcatatcaggagttatatcaaacttgaaggaactaaaatagatagccagggagttataacttgggttttctggtgctaaacatgtagtcatatagacaatgaaaaacaaaaatctcctaccatttactatgcagtagcagcagcatctgagttattgactctcccagctcactaacgaacaagctcttttggctaaaaaggaggataaggctccaagctgagaccaaacctcttgtcagagtctgaggtctaaatcaagcttgagtggctcctgtggtgactgtggctgttttggaaccaagcgaaagctgcctggccacaccatatccgcttgatcctcggtccagaaaggtgtccatgccatcccaatccttgcatatgcatcatgaaagctcggtgccaccatgataccatctctgcatggcttgtgcacaagtgagtgtggctgaacaccaagtgatcgacacatagttagtgtgttcatnnngaatcccattattgccatcgacctctgagattggtatcttctggcagcacccctttccctcttgtgagcgttctgatggcctcctagtgcctgtgagctaaagaacttcctcatgcagnngttgcatgaaaaaactttggcagtggctggtgcctcgtg

SEQ ID NO: 37 - DNA, 629 bp, Glycine max
  misc_feature (457)..(459): n is a, c, g, or t
tgttagactcacatggacagatacatacgactactccctctacgatgaacgtacaacgctactaataaaaacctaagcatgctgagggaaaattaaaatgaaaaaacatgggagtagtagtagataatactagggaacataaaacgacgtcgaaagaaacctagtcagggttcattcattattcatagccacaagggtgggggttcgttcaagtcgatggggatgccgcgcctaacgagacccaaaaacgacgccgttggagaactctccaccacctgaactcccccggcgtgggggcttcggtgagcgacgatggcggcggcgggtggaaaaggtagttgcggcggttcaatgtctctgagaacggcggtgtggaggaactcgctgaggacgaggctgcgaggggggacgtggggccagcggtgatcggaggaagggacgttgaggtcgaggcagnnnggcggcgccggnnngaagttggtcttggccttggcgccgcggagggagcgggcggcgccgtnnnnngcgagggcnnnnnnctcgggagtancgaatgtgcccagcnngacacgtgtcttctnnnacggatctcntatttctgcagcgtaacgtccccatggtcgcttcct

SEQ ID NO: 38 - DNA, 647 bp, Glycine max
ttaattaattatccagttgtatactgcggctagctagctagatcacttgcattaatagctaataatttggtaagctaatttcatcaagatcatcatccaatgacaagaatacttcatccaagcacgacagctctgttccattatcatcgtcgccattgcgctcataaacacgacatatcacccatttattgtattcctgaatcaaacacaaaaggaaaaataaaatcagcaatcaaatcaatgtgttgtacaatagttttccaactcgaatatacgtcagtggtaactgttcacacgactagctattttaaaaaagttatatgtatttttttaattttaatcacttatgtattagctaatagtttatatttttaattctcatttcttactataagaagagttcatgtttgatgtgcttcctgattatgtatgaaaatatgtatactgactatttttggttgtgattttcttttggatgatctggtagaggaagaagaatctgatagacgatattcttgcattatccaattggttttgataccagcagcaggggcttctcccacatggaacacaaaatatttcttcatgccaactctcttgttgcttgtgcttgaaaccactggttcttctattcccgttggtttcca

SEQ ID NO: 39 - DNA, 590 bp, Glycine max
  misc_feature (36)..(38): n is a, c, g, or t
tcctatttctctgcgatttcacctaatcatttttcnnngacctaatatgaatataattttccgctcttgaattcattattcaaccctaccaattcccatcctctccaacaccgagttcctaatggcatcggtttgcatacacacaccattacgttccagtacagacatgtaatttctcacttccactcgtatcatctcttgcatcaccttcataaactcttggctgaacaactgcttttgggaaccaagcatcggtatctcctggaacgggccgcaactgggcccctgattcggcccaggcccattattagccccatcgcacgaatcgaatcccggtagagacaaactcaataacgtggcaggatcagttactgaagaagacgcggtttccgcaaggtggttagagggagctaccgtcacgctcacgcttcttgcaagaataatgggtaaacccgaatcgctcaaatcggatccggattgactacccgaaacggtggcggatctcttcnnnngcnnnngctcggnnnnnggctcggagcacttcctcttgnnngtggaattcnnntggttcttgatggcgttgtcggtgc

SEQ ID NO: 40 - DNA, 465 bp, Glycine max
gggactgagggtggttctctcagaaatgttcctgtgggtggtggctctagaaagaacaagagatcaaccccgccagcaccaccatcagcaccagcaccaacaaagaagctttctgatctcgcaacaccaaatttccctcaatctgcttctcaggaccctaagatccaccaaggccaagaccttaacctagcatatccaccagctgaggactacagcactgtctccaagttcattgaggttccttacagcactgaattagacaagggtactactggtcttcatcaaaaccctacttcctcatcaacaacaacatctgcatcttctcagttgtctgccatggagcttctcaagactgggattgcagctgcttcctcaaggggtttgaactccttcatgccaatgtataattcaacccatgggtttcccttgcaggactttaagccaccacatggccttaacttcagc

SEQ ID NO: 41 - DNA, 515 bp, Glycine max
  misc_feature (491)..(491): n is a, c, g, or t
acgagggggcaagctgtatgctcttattacagaatctacggaatgtgtaagtttggcccaacatgcaaatttgatcatccagttttgactatccctcagaactatggcttgacctcacctgctatgaatgtacttgatactcctctcaccagggggttatcaaatgtacaaccacctgagacatctccttctaaattaagtgacaagaagcttcagcattctgatgcaaaagctgctacagaagattcatccaaacaagatgatactacattgaattccttcccggcttccgcagagcctttacacaactaaactacttgaagtttgattcttttttcttttggatattttgttttcccccacattttcaggagaagtatatcatacataccatctattcccccccttttcacctgcaattctgtggagttggatagtttgttcagagatttttgtgacaaccactagcttgtggggaatggaaggcattntttgtgttgaataaaatgcangta

SEQ ID NO: 42 - DNA, 604 bp, Glycine max
  misc_feature (12)..(12): n is a, c, g, or t
aacttatattcntctttggcataaacattactgatgaaagcaattgttagattgaatcaatgacaaagaagagtccaaaagcaatgaacaaaatttgtctgtacagagtagcagaaacaaaatcccaatcccacctatatactaaaaaaaaacataaaatgaaaattaaactaactggtttgaattcataatcatattcgcgatataccagcaacaaattcaatttcaatcaatttaaaataaaattctgcaaacaaatctacgcggtttcatcatcttcccctgtaaacaaacgcggcctcttggcggttaccggcaacgggctttccacctcttgttcaccgcctaccttcttcttcccgaaatcaaaccccatcggcgaccagaactccgtcagcggcgccggaaggttcaggtcaaacccacggagcgtgtgggaggnngaagcagnnnnnnnnnnnnnnntannngcgccgctgctgttgtttccgttggcgttgctgttactgtggttgttcccgccgtcgtannggcagcgcttgtggccacccaaggcctgaccggtagggaaactcttgtgacagatggaacactcgtgcatcnt

SEQ ID NO: 43 - DNA, 395 bp, Glycine max
gcaccaggccgaaccgtgccgccggaagcggctattggaaagccaccggcgccgataaggcgatcggaaaaccgaaagcgctagggatcaagaaagctctggttttttacgccggaaaagcccccaaaggagtgaaaaccaattggatcatgcacgaatatcgcctcgccaatgttgaccgatctgcctccaagaaaaacaacaacaacttgagggtatgtcccaaattgtaatttcactagtttttcttttatgggcaaatattaattttgttagaatgttagttttcttgcaatcttttttctgtttcccttattcaattcaccttatatctccaatttgactaaaattttgattagaaaaggattagaattgcataagttttgatctttcag

SEQ ID NO: 44 - DNA, 622 bp, Glycine max
gccacacttacatggtcaaggagtttcatcttcacctgctgttgggactcattcaagcatagaatcaccaaccaaattatctggaaatactgatcagggtttaatgaagaaatcaaaagggtttgatgggcttgcaatgtcaataggcaattgcaatgctgagagtgctgagcatggagctgagaacaggcagtcacagagtgtggatactgagggttacagcgacggaagtgatggcaacactgcaggggctaatcaaacaaaaaggaaaagatgccgagagggaacactgaccactggtatgactcttattcactgttgcatcttgacgaacaaaagagttgtgttggattagaattatacttttggtgttatcactgtgcaatgatgtcaactagatcattgagtatggtactatggtcttgataagttgatattatcctattacttatgcacagttaatgaagttacccgagttttaaccttctgtttagttggacaaatttgatacttggatgggctttttacctggttttcatacttaatacagtcaagcttttggtgtcctttgtatgcaattatcaagaaaatgacatacttgcatgcagaatgtgatatttaa

SEQ ID NO: 45 - DNA, 622 bp, Glycine max
tttctcttgaagcctctccaatttgccttcacttcttaataataataataatattaattcctcttttcttgtaccaaaccaaattgatattttgaactcaatggaaggtagcacaacaagctctgaactccctcctggctttaggtttcacccaacggacgaggaactgattgtgtattacctttgtaaccaagccacctcaaagccatgccctgcatctatcatcccagaagtggatctctataagtttgatccttgggaattgccagataaaacagaatttggagaaaatgaatggtatttctttagtccaagagacaggaagtacccaaatggggtgaggcctaaccgagcaactgtgtctgggtattggaaggctactggcacagacaaggcaatctatagtggatctaagaatgttggggtgaagaaatctttagtcttttacaagggtaggccaccaaagggtgccaagactgattggatcatgcatgagtatcgattggctgaatcaaaaataccagctagcaggaaaattgggtccatgaggctggatgactgggtcttgtgcaggatctataagaagaagagcatggtaaaagcattggagcacaaagaggca

SEQ ID NO: 46 - DNA, 518 bp, Glycine max
ctgatatcaatttcaatctgagtgattatgaggatgaccttaaacagatgcagaatttgtccaaggaggaattcgtacatatactgcgtcgccaaagtactggtttctcaagaggaagctctaaataccgaggagtaacactccacaagtgtggccggtgggaagctcgaatgggacaatttcttggcaaaaagtgaggacatatttttatatatcaacttacgtagtgatctactgtggttatgaaatattatgaatttaagcacctattgggctgcaaagtgaaattggtttcagcttaattctcaatgcatctttatgcttttttccatgtttggagaaacatgtctaagatagtaatatagagatgcgttttggttatagagattgcaatgctttcatgcaggtatatatatcttggactattcgacagcgaagtagaagctgcaaggtcctagtgatacatgaattacattctacccgcactgaatttatttacatgaattatcaaagctcat

SEQ ID NO: 47 - DNA, 746 bp, Glycine max
  misc_feature (6)..(10): n is a, c, g, or t
ggtttnnnnnttaatattctttaatcaatttctaaattaaagcaaaaacaaaaagagagaagctttaaattaaagatcatatatacctggggtgctgcgtgttcttcacaactttctgtccgtacttcctccacttgtagccatcgtccaatacatccacatcgctcatggtcttgaagcaaaacctaggctctctcaccttccttcttgccttgattttcttcatcttcatggctgaaattcggaaatgatgatcatcaaatccacttcttttactgctgaagcattcagccacttctcccaatgcccatggatttacacttgatctatgcaaggataggaattgacctcctccaaagctagaagtgaggtcttctcttgacttttgagcagtggttgagaatagggtttctgctgttagatttgatgcagaatcttgttgagatatagcaagtgaagaaggtgctatggaactaaaggctttcaaagaggattgatggcaacccaaaggagggaaagtaaggtttgttggaaaggaaaagaaacccatctgtgtaggtatctggtcctgctcatcgaataagctgtggtgaacaatgggatgggaagttgttgacatcacttcttatgatcttttccctttctccttctcccttctctcttccaattatctttatatagaggttgatgatgtttctagaggaagaggtttggtgaaaattgaaataggtggtttctctcctggt

SEQ ID NO: 48 - DNA, 463 bp, Glycine max
gctcctttgagcatagagacaccacccaaatcatctggaaatactgatcagggtttaatgaagaaattgaaagagtttgatggacttgcaatgtcaattggcaatggccatgctgaaagtgcagagcgtggaggtgaaaacaggctctcacagagatggagaagggaaaactgagatacaaggcagtccaatttccaaagagactgcagcttctaataagatgttgggagttgtccctgccagtgttgcaggaacaatagttggacatgtagtttcttcaggtatgaccactgcactggagctgagaaatccttccagtgttcattctaaaacaagtgccccacaaccttgtccagtattgcctgcagaagcttgggtacagaatgagcgtgagctgaaacgggagaggcggaaacagtcaaatcgagaatctgctagaaggtccagactaaggaagcatgct

SEQ ID NO: 49 - DNA, 701 bp, Glycine max
  misc_feature (693)..(695): n is a, c, g, or t
ccccaccagcaattagaacagccaatgtccacccaaacatgcagagatgagacccatctaaatatgggactaattaatttcttcagggggcatcagagagttcccatgacaaaaagaaatgaaagaattttatgatgtggaaaaggataattttaattcggagtctcatatcaaaactcacgcagtatagaaggcaaagctaaaccttcaataaaaactccaacagcctcttatcgttgaccctctttttttatgtatcatcatcatgtagtattattatgtagtggtaaaactcctcccatgtatacttttatctgcaaaattcatggtggagagtagtatcctatacatcaacttttcctgctttgtgcatatgacttttccaactaaaactctcggatcttgcatcttcatcatcttccatgctgtcaccttcagttggggatgttcctccccgggaccctgcagccaaatgaagaggactctgaggagaacctgaccctgaactgctaacttttgaagagtcattgtactgatcttgagacatccacaaacctccaagaaccacaactggctggttagaacttgcagctggaactctccgcgtatgaagacgatatttttgtaaatggctcttcacttcatcgttagtcaggccatcaacttgcataagctctctaatttgctttggtnnngctact

SEQ ID NO: 50 - DNA, 556 bp, Glycine max
agagagagagagaactagtctcgagttttttttttttttttttgaaaagttgaagcaggattcttaacatcacttgcaaaactaagttctgaacaaaagtgctcaagtagcctacgttttctattctaaggtactggttaatattacaaacaaatgacatgtaaaatcacaagggcaattacaattgagggttaatgacatagagttagtataacaaagaaaaaaactttatatcctattctcaattaagagacctggccattgataagaaaattctagctgcagattcgtcttaattaagaatctatcaaattttgccgttaccatcaaacactaaactgaggaactgaaatgcgttgctttctcatccaaggttagcaactgcttctggaccacacaaccgctccaactcttccttgatagaattattttctgatgtaagcttctcgcattcttcagaaagtctctgaagctcatctctgagagtttgattctcacctcccagcgactccaccctcttttgtaactcttcacactcagcctgcttgcgtaatct

SEQ ID NO: 51 - DNA, 578 bp, Glycine max
acgaaaagcgaggtggatcatctggaagatgggtacagatggagaaagtacggtcaaaaagctgtgaaaaacagcccctttcccaggagctactatcgttgcaccagtgtttcatgtaatgtgaagaaacgcgtggagcgatctttcagcgacccaagcattgtggtgacaacctacgaaggccaacacacgcatccaagcccagttatgggtcgctccaacaactttggtacggtaatgtctggatctgctggaaactacatgtcccaatattatcatcagcaacaagtccacgtcaatgcattgtcttctttgggtttcctctcttcttcttcgtcttcaaggaatgccactttttctcaagagactgccttgttaagtgactatgggcttcttcaagatgttgttccttcacatatgttgaaagaagactagatgataataacttcactactgtttctatgtgatttcttataattagtttttgtaggagaatatagatgaggactatgttgctgcactgggatatttaagttttagttttcattaccatcatgaaaaaaaaaaaaaaaaaactc

SEQ ID NO: 52 - DNA, 758 bp, Glycine max
ctatgggaaggcaacctagatgtgacaaacttggggtgaagaagggtccatggactgctgaggaagacaagaagctcatcaacttcattctcaccaatggccaatgctgttggcgcgccgtgccaaagctcgctggcctcaagcgctgtggcaagagttgtcgccttcgttggacgaattatcttcgtccagacttgaagagagggctactcacagaggcagaggagcaacttgttattgatctccatgcccgtcttggcaacaggtggtccaagattgctgccaggttacccggaagaacagacaatgagattaaaaatcattggaacacccatatcaagaaaaaactccttaagataggtattgatcccgttactcatgagcctctcaaaaaacaagctgcagcatcttctcaggatagttcatcctcccctgcagagcatttgccgcaacctggcaataaccttgatgatcaggtcaaagaaactgaagggattcttctcaactcagaggagaattcaagttcatcacaagctgaaaattcttctggggatgattcacttttgctagatagcatttgcagcgacgattctctattaaacagcatgtggctggatgaaactcctctggtggaagcattatgggacacaacacctaaccttgagaatactagtaataacatgggtttgttaccatcttgggaggataattgtgcttggttattggactgtcaagactttggtattcatgattttggg

SEQ ID NO: 53 - DNA, 480 bp, Glycine max
gtttgagaaccatgcagaaataattaaactataagctacctaccttccaatcatcatcactcataattttaattaattttttcctaccatacagtactattagtattaattagttctttgtcttatgtttttttttttttttatatagaataggatatcaaaagatttataataatttatatacattgagctagatcgtttgacaccttattttgttaattagttactttgaatttttttccttaattactttgatgattcctttgcagatggtcaagaattgctcgcaggctaccagggcgaactgacaatgagattaagaattattggaggactcacatgaggaaaaagaaggctcaagagaaaaagcatgcatcagcagcatcgcctgtttcttctagttgtcaatcttcactgtcctcaaacaaccatgttgtggattcacatgcttccaaagaggttggagaacagagttgttat

SEQ ID NO: 54 - DNA, 527 bp, Glycine max
atagtttcagctttgacacacgtggtgtcttcagggtctggccagaggagcaccgaatggacccagcaaagtggttttcctatgatgtctgcttcttctctttcacgtttgtctgctttctcttcttcttctccttctccttcttctggagcctcttgggttggccacaaaagaggcagagaagaagaagagaatagtacttcacataacttgatgcaacaacaacaacaaagtgctccaagactcttcagaaacattggtgacttcatggtgccttctcaaggagactcatcatcagtgacagaagaagcccccacctccacaactacaactgtaaccgccgtcactgaaaacccaccaggaggtggggaaaggaggagaaagtacagaggagtgaggcagaggccatggggaaaatgggcagcagaaatccgtgatccacacaaagcagcaagagtttggctaggcacatttgacacagaagaagcagcagcaagagcctatgatgaagctgcattgaggttcag

SEQ ID NO: 55 - DNA, 609 bp, Glycine max
ttcataacataaagagtatagagcagcaaacgtaaaagtggatccaagaaggctacatggcaagcttccactatcttcacaagcatcagaggagaaagaggacacagtagaagagtttgatgcttataataattatgcatcttggtcttcagaaactgacatgtctgaaatggtttctgctcttgctcaagttatgggaactaccaatcggaacccatacatgccacaatcaacctcctttcctttctcaacttccgtggtcaaagatcaacctcaaccgtacccatctcaacctcctccagatcaagaggtggcaaagaagccacactacagaggagtgaggcagagaccatgggggaaatgggcagcagaaatccgtgacccaaaaaaagcagccagagtgtggctaggaacctttgaaacagcagaggatgcagcacttgcatatgacaaagcagcactcaagttcaaaggcacaaaagctaagctcaatttccctgaaagagttcatcaagatgttccctttatgcaacaacaccaacaacgttcatcaaacagaaacgtttttcccatccatgcaacttccaccactagtagtgctactgggtc

SEQ ID NO: 56 - DNA, 560 bp, Glycine max
aacaaatcagggaattaatgaaggttgatggcctcaccaatgatgaagtcaaaagtcatttacagaaatttcgtcttcacactagaagaagccccataatccacaacagtgcaagttcacaagcaggctctttatttcttgtggggaacatttttgtgcagccaccagaatatgctacctcatcagcatctggaggggaattgaccacagctacccctgctgctccaactggaatatatgcacctgtggctgcacaccttccagctggcacccacacatcaccaattatgaagcattcacattcaccttctgaggaaaagaataatcatagtgttcattcaaactcaccggcctcatcatcctccacacacaccatcacaacttcaccacctgcctaattgattttgatcaatatgttgtaaaaaaaattgtaaacatattgtagttcaaagaaagcctatgtgatattgtttgttatcctctttataattagtgtatgtttagattaatttattttaagaaaataatctttttttttttctgtattttactcaaaaaaa

SEQ ID NO: 57 - DNA, 458 bp, Glycine max
ggagttgccgtctccgttggacaaactatctccggccagatataaagcgaggtcggttttcatttgaggaggaagagacaataattcaactacatagcattcttggaaacaagtggtctgcgattgcatctcgcttaccaggaaggacagacaatgaaataaagaactattggaacactcacattagaaaaaggcttctgagaatgggaattgaccctgtgacacacagtcctcgacttgatctattggacatcaattccattctaaacgcatctttctatggttcatcacaaatcaacatccaaagactccttggcatgcaccagtacatggtgaaccctgagcttttaaaattggcttcttcactcttctccacctcacagcagcaacacaatgaccctgatgacatgcctgctcaaaaaggtcaattacagaaccagctttgtgatcctcaaatt

SEQ ID NO: 58 - DNA, 557 bp, Glycine max
attttcatcatcttcttcttctttttcatcaatcatcaccagtggttcttttccttctgagaagatcgaattgtgttttgcaaaaatgggagttccagagaaagaccctctttcccaattgagtttgcctcctggttttcggttttaccccaccgacgaggagcttctcgttcagtatctgtgccgcaaggtcgctggccaccatttctctcttccaatcattgccgaaattgacttgtacaagttcgacccatgggttcttccaagtaattatcaaatcgctcacccatctccatttttttttaagttaattactttgttctaaattgaatagatttgattgattaattttgcaggtaaagcgattttcggtgaaaaagagtggtactttttcagccccagagacaggaaatacccgaacgggtctcgacccaacagagtagctgggtcgggttattggaaagccaccggaaccgacaagatcatcaccaccgaaggtagaaaagttggcataaaaaaagccctcgttttctacgttggcaaagcccccaagggca

SEQ ID NO: 59 - DNA, 456 bp, Glycine max
aaatagactgtaatatatcagaaaatttgtacaccatgctttgaatatgtggcatcaaatttgattttgatgatatataaagtcaacttttataagctgattttcccacaaccactcccaaacaaccgctgaagatgtggctacataattatcggaaaggtgcactagtttctagaccaaattattactaagctgtacacgtaatgacgcgggtatcctcctccttttgcgttattgctgcttcttgtgtgaacacgtacactttgcccgattgtccgaaagagtctgaacttggttttgaaggtctttaatgtactcaacagccaagtccaacatgtcagctgtgtttgtttgcttatccatgttgggaacgagatcttgtagcttcctcattcgctcactaattttagttcttctaaccctctctgcaatgcttcttgggtgagtgcctcgtgc

SEQ ID NO: 60 - DNA, 441 bp, Glycine max
agttttttttttttttttttattactaaatcatccaatctctattactatgttgacatacaaaaggatcccgtcaaatttgtgcagaccctgtatccatatctttctcaaccgtggcataaataacataatgatttctgcctaaggatcacttgaaaatgattttaacactatattttaagaattaaagaatggaccaataattggagcaggccaatatctggacctggaacaatactccttatactcgggggaacgcttaacttgaataattctaactgttgtttttcatcgaccttttattgtcgcatgcccagaatcgccaagatgtttatgtaatttattccttgtctcaaactcctctccacatgcatcacaaatattgccaacattctttgatgccacttttccttttcttcctttggatgaaacttttgcttta

SEQ ID NO: 61 - DNA, 482 bp, Glycine max
aaacgaggcatggcatcattattagttgcagtgccaccaaacacttgagaatgttgatcactgagtggcgtcgccgaggcgtggaacaatgatggacgattagtgaggtaaccaaattccttagagaaatgaaccctttctggaaaattaagcttagctttggttcctttgaatctgagagctgcttcatcgtaagcgagtgcagctgcctcagcagtttcaaaggttcccagccaaactctcgctgctttctttgggtctcgaatctccgatgcccacttcccccacggtcgttgtctcactcctctatagtgccttcttacgtttccttcacatacacacacacatgtccatacatttcaacaaaacaatttgctcccatctttgttacactattttttatttttttttaacaagttatgatatttagacatccatttttctcaaacatctatctgatactaatcatttctctttaaaaa

SEQ ID NO: 62 - DNA, 473 bp, Glycine max
gtgaagccaccaccacattttcccgaacaagaacaactccagtgtccacgttgtaactccaacaacaccaaattctgctactacaacaactataacctctcgcagccacgccacttttgcaagaactgtagaagatactggactaaaggtggtgccttgagaaacatccctgttgggggtggaagcagaaagaccacaaaacgttcttcatcaacctcaaaacgctcttctgcaccatcttcttcttcctctgcagtttcggatcctgacccgacccgtaattgcaccaacccggttgatcaagaccaaagggtgctcaacattggtggtgggagcttcagttccctcttggcttcatctgggcactttggaaccctcctggagggtctgaatccaactgggtcgagtctcaaaatgggtgaatttggggagggtgtgagttctgatccgggtttgaatttggtttcgggtca

(19) United States
(12) Patent Application Publication
(10) Pub. No.: US 2009/0210968 A1
(43) Pub. Date: Aug. 20, 2009

(54) Drought Responsive Genes In Plants And Methods Of Their Use

(76) Inventors: Henry T. Nguyen, Columbia, MO (US); Babu Valliyodan, Columbia, MO (US)

Correspondence Address: LATHROP & GAGE LC, 4845 PEARL EAST CIRCLE, SUITE 300, BOULDER, CO, 80301, US

(21) Appl. No.: 12/138,392

(22) Filed: Jun. 12, 2008

Related U.S. Application Data
(60) Provisional application No. 60/934,321, filed on Jun. 12, 2007.

(51) Int. Cl. C12N 15/29; A01H 5/00

(52) U.S. Cl. 800/287; 800/278; 800/298; 800/312; 800/320.1; 800/320.2; 800/320.3; 800/314

(57) ABSTRACT

Microarrays are employed to analyze soybean transcriptions under water stress conditions in both root and leaf tissues at vegetative stage. Novel drought responsive genes may be thus identified and may be used for enhancing drought tolerance in soybean or other plants through genetic/metabolic engineering. This disclosure pertains to nucleic acid molecules isolated from Soybean that encode polypeptides that may be important for drought tolerance. The disclosure also relates to methods of using these genes from soybean in transgenic plants to confer the desired agronomic traits, and to use such genes or regulatory elements thereof to assist germplasm enhancement by molecular breeding or to identify other factors or chemicals that may enhance a plant's capability to grow under drought conditions.

### RELATED APPLICATIONS

This application claims priority of U.S. Provisional Application No. 60/934,321 filed on Jun. 12, 2007, the contents of which is hereby incorporated into this application by reference.

### BACKGROUND

1. Field of the Invention

The present invention relates to methods and materials for identifying genes and the regulatory network in an organism that control the organism's response to various environmental conditions or stress. More particularly, the present invention relates to the plant drought-responsive genes and their use.

2. Description of the Related Art

Drought is one of the major abiotic stress factors limiting crop productivity worldwide. Global climate changes may further exacerbate the drought situation in major crop-producing countries. Although irrigation may in theory solve the drought problem, it is usually not a viable option because of the cost associated with building and maintaining an effective irrigation system, as well as other non-economical issues, such as the general availability of water (Boyer, 1983). Thus, alternative means for alleviating plant water stress are needed.

In soybean, drought stress during flowering and early pod development significantly increases the rate of flower and pod abortion, thus decreasing final yield (Boyer 1983; Westgate and Peterson 1993). Soybean yield reduction of 40% because of drought is common experience among soybean producers in the United States (Muchow & Sinclair, 1986; Specht et al. 1999).

Mechanisms for selecting drought tolerant plants fall into three general categories. The first is called drought escape, in which selection is aimed at those developmental and maturation traits that match seasonal water availability with crop needs. The second is dehydration avoidance, in which selection is focused on traits that: lessen evaporatory water loss from plant surfaces or maintain water uptake during drought via a deeper and more extensive root system. The last mechanism is dehydration tolerance, in which selection is directed at maintaining cell turgor or enhancing cellular constituents that protect cytoplasmic proteins and membranes from drying.

The molecular mechanisms of abiotic stress responses and the genetic regulatory networks of drought stress tolerance have been reviewed recently (Wang et al 2003; Vinocur and Altman 2005; Chaves and Oliveira 2004; Shinozaki et al. 2003). Plant modification for enhanced drought tolerance is mostly based on the manipulation of either transcription and/or signaling factors or genes that directly protect plant cells against water deficit. Despite much progress in the field, understanding the basic biochemical and molecular mechanisms for drought stress perception, transduction, response and tolerance remains a major challenge in the filed. Utilization of the knowledge on drought tolerance to generate plants that can tolerate extreme water deficit condition is even a bigger challenge.

Analysis of changes in gene expression within a target plant is important for revealing the transcriptional regulatory networks. Elucidation of these complex regulatory networks may contribute to our understanding of the responses mounted by a plant to various stresses and developmental changes, which may ultimately lead to crop improvement. DNA microarray assays (Schena et al. 1995; Shalon et al. 1996) have provided an unprecedented opportunity for the generation of gene expression data on a whole-genome scale.

Gene expression profiling using cDNAs or oligonucleotides microarray technology has advanced our understanding of gene regulatory network when a plant is subject to various stresses (Bray 2004; Denby and Gehring 2005). For example, numerous genes that respond to dehydration stress have been identified in Arabidopsis and have been categorized as “rd” (responsive to dehydration) or “erd” (early response to dehydration) (Shinozaki and Yamaguchi-Shinozaki 1999).

There are at least four independent regulatory pathways for gene expression in response to water stress. Out of the four pathways, two are abscisic acid (ABA) dependent and the other two are ABA independent (Shinozaki and Yamaguchi-Shinozaki 2000). In the ABA independent regulatory pathways, a cis-acting element is involved and the Dehydration-responsive element/C-repeat (DRE/CRT) has been identified. DRE/CRT also functions in cold response and high-salt-responsive gene expression. When the DRE/CRT binding protein DREB1/ICBF is overexpressed in a transgenic Arabidopsis plant, changes in expression of more than 40 stress-inducible genes can be observed, which lead to enhanced tolerance to freeze, high salt, and drought (Seki et al, 2001; Fowler and Thomashow 2002; Murayama et al. 2004).

In summary, the production of microarrays and the global transcript profiling of plants have revolutionized the study of gene expression which provides a unique snapshot of how these plants are responding to a particular stress. However, no transcriptional profiling or transcriptome changes have been reported for soybean plants under water stress conditions. A well designed analysis of gene expression in soybean grown under drought may help illuminate the regulatory networks in soybean under these adverse conditions, which may further lead to development of new soybean plant that can better tolerate drought conditions than conventional strains.

### SUMMARY

The instrumentalities described herein overcome the problems outlined above and advance the art by providing genes and DNA regulatory elements which may play an important part in the drought responses mounted by a soybean plant grown under water deficit conditions. Methodology is also provided whereby these drought responsive genes may be introduced into a plant to enhance its capability to grow and reproduce under water deficit conditions. The regulatory elements may also be employed to control expression of genes that are not yet identified but may prove beneficial for enhancing a plant's capability to grow under drought conditions.

More particularly, microarray experiments are conducted to analyze the gene expression pattern in soybean root and leaf tissues in response to drought stress. Tissue specific transcriptomes may be compared to help elucidate the transcriptional regulatory network and facilitate the identification of stress specific genes and promoters. For purpose of this disclosure, genes whose expression are either up- or down-regulated in response to drought condition are referred to as Drought Response Genes (or DRGs). For purpose of this disclosure, a “DRG protein” refers to a protein encoded by a DRG. Some DRGs may show tissue specific expression patterns in response to drought condition.

The microarray experiments described in this disclosure may not have uncovered all the DRGs in all plants, or even in soybean alone, due to the variations in experimental conditions, and more importantly, due to the different gene expressions among different plant species. It is also to be understood that certain DRGs disclosed here may have been identified and studied previously; however, regulation of their expression under drought condition or their role in drought response may not have been appreciated in previous studies. Alternatively, some DRGs may contain novel coding sequences. Thus, it is an object of the present disclosure to identify known or unknown genes whose expression levels are altered in response to drought condition.

In order to generate a transgenic plant that is more tolerant to drought condition when compared to a host plant, the expression levels of a protein encoded by an endogenous Drought Response Gene (DRG) or a fragment thereof may be altered to confer a drought resistant phenotype to the host plant. More particularly, the transcription, translation or protein stability of the protein encoded by the DRG may be modified so that the levels of this protein are rendered significantly higher than the levels of this protein would otherwise be even under the same drought condition. To this end, either the coding or non-coding regions, or both, of the endogenous DRG may be modified.

In another aspect, in order to generate a transgenic plant that is more tolerant to drought condition when compared to a host plant, the method may comprise the steps of: (a) introducing into a plant cell a construct comprising a Drought Response Gene (DRG) or a fragment thereof encoding a polypeptide; and (b) generating a transgenic plant expressing said polypeptide or a fragment thereof. In one embodiment, the Drought Response Gene or a fragment thereof is derived from a plant that is genetically different from the host plant. In another embodiment, the Drought Response Gene or a fragment thereof is derived from a plant that belongs to the same species as the host plant. For instance, a DRG identified in soybean may be introduced into soybean as a transgene to confer upon the host increased capability to grow and/or reproduced under mild to severe drought conditions.

The DRGs disclosed here include known genes as well as genes whose functions are not yet fully understood. Nevertheless, both known or unknown DRGs may be placed under control of a promoter and be transformed into a host plant in accordance with standard plant transformation protocols. The transgenic plants thus obtained may be tested for the expression of the DRGs and their capability to grow and/or reproduce under drought conditions as compared to the original host (or parental) plant.

Although the DRGs disclosed herein are identified in soybean, they may be introduced into other plants as transgenes. Examples of such other plants may include corn, wheat, rice, and cotton. In another aspect, homologs in other plant species may be identified by PCR, hybridization or by genome search which may share substantial sequence similarity with the DRGs disclosed herein. In a preferred embodiment, such a homolog shares at least 90%, more preferably 98%, or even more preferably 99% sequence identity with a protein encoded by a soybean DRG.

In another embodiment, a portion of the DRGs disclosed herein are transcription factors, such as most of the DRGs or fragments thereof with SEQ ID Nos. 1-62. It is desirable to introduce one or more of these DRGs or fragments thereof into a host plant so that the transcription factors may be expressed at a sufficiently high level to drive the expression of other downstream effortor proteins that may result in increased drought resistance to the transgenic plant.

It is further an object to identify the non-coding sequences of the DRGs, termed Drought Response Regulatory Elements (DRREs) for purpose of this disclosure. These DRREs may be used to prepare DNA constructs for the expression of genes of interest in a host plant. The DREEs or the DRGs may also be used to screen for factors or chemicals that may affect the expression of certain DRGs by interacting with a DREE. Such factors or chemicals may be used to induce drought responses by activating expression of certain genes in a plant.

For purpose of this disclosure, the genes of interest may be genes from other plants or even non-plant organisms. The genes of interest may be those identified and listed in this disclosure, or they may be any other genes that have been found to enhance the capability of a host plant to grow under water deficit condition.

In a preferred embodiment, the genes of interest may be placed under control of the DRREs such that their expression may be upregulated under drought condition. This arrangement is particularly useful for those genes of interest that may not be desirable under normal condition, because such genes may be placed under a tightly regulated DRRE which only drives the expression of the genes of interest when water deficit condition is sensed by the plant. Under control of such a DRRE, expression of the gene of interest may be only detected under drought condition.

It is an object of this disclosure to provide a system and a method for the genetic modification of a plant, to increase the resistance of the plant to adverse conditions such as drought and/or excessive temperatures, compared to an unmodified plant.

It is another object of the present invention to provide a transgenic plant that exhibits increased resistance to adverse conditions such as drought and/or excessive temperatures as compared to an unmodified plant.

It is another object of the present invention to provide a system and method of modifying a plant, to alter the metabolism or development of the plant.

In one embodiment, a gene of interest may be placed under control of a tissue specific promoter such that such gene of interest may be expressed in specific site, for example, the guard cells. The expression of the introduced genes may enhance the capacity of a plant to modulate guard cell activity in response to water stress. For instance, the transgene may help reduce stomatal water loss. In addition, other characteristics such as early maturation of plants may be introduced into plants to help cope with drought condition.

Preferably, the transgene is under control of a promoter, which may be a constitutive or inducible promoter. An inducible promoter is inactive under normal condition, and is activated under certain conditions to drive the expression of the gene under its control. Conditions that may activate a promoter include but are not limited to light, heat, certain nutrients or chemicals, and water conditions. A promoter that is activated under water deficit condition is preferred.

In another aspect, a tissue specific promoter, an organ specific promoter, or a cell-specific promoter may be employed to control the transgene. Despite their different names, these promoters are similar in that they are only activated in certain cell, tissue or organ types. It is to be understood that a gene under control of an inducible promoter, or a promoter specific for certain cells, tissues or organs may have low level of expression even under conditions that are not supposed to activate the promoter, a phenomenon known as “leaky expression” in the field. A promoter can be both inducible and tissue specific. By way of example, a transgene may be placed under control of a guard cell specific promoter such that the gene can be inducibly expressed in the guard cell of the transgenic plant.

In another aspect, the present disclosure provides a method of generating a transgenic plant having an altered stress response or an altered phenotype compared to an unmodified plant. The coding sequences of the genes that are disclosed to be upregulated may be placed under a promoter such that the genes can be expressed in the transgenic plant. The method may contain two steps: (a) introducing into a plant cell capable of being transformed and regenerated into a whole plant a construct comprising, in addition to the DNA sequences required for transformation and selection in plants, an expression construct including the coding sequence of a gene that a operatively linked to a promoter for expressing said DNA sequence; and (b) recovery of a plant which contains the expression construct.

The transgenic plant generated by the methods disclosed above may exhibit an altered trait or stress response. The altered traits may include increased tolerance to extreme temperature, such as heat or cold; or increased tolerance to extreme water condition such as drought or excessive water. The transgenic plant may exhibits one or more altered phenotype that may contribute to the resistance to drought condition. These phenotypes may include, by way of example, early maturation, increased growth rate, increased biomass, or increased lipid content.

In accordance with the disclosed methods, the coding sequence to be introduced in the transgenic plant preferably encodes a peptide having at least 70%, more preferably at least 90%, more preferably at least 98% identity, and even more preferably at least 99% identity to the polypeptide encoded by the DRGs disclosed in this application. In an alternative aspect, DNA sequence may be oriented in an antisense direction relative to said promoter within said construct.

In accordance with the methods of the present invention, the promoter is preferably selected from the group consisting of an constitutive promoter, an inducible promoter, a tissue specific promoter, and organ specific promoter, a cell-specific promoter. More preferably the promoter is an inducible promoter for expressing said DNA sequence under water deficit conditions.

In another aspect, the present invention provides a method of identifying whether a plant that has been successfully transformed with a construct, characterized in that the method comprises the steps of: (a) introducing into plant cells capable of being transformed and regenerated into whole plants a construct comprising, in addition to the DNA sequences required for transformation and selection in plants, an expression construct that includes a DNA sequence selected from at least one of the DRGs disclosed herein, said DNA sequence may be operatively linked to a promoter for expressing said DNA sequence; (b) regenerating the plant cells into whole plants; and (c) subjecting the plants to a screening process to differentiate between transformed plants and non-transformed plants.

The screening process may involve subjecting the plants to environmental conditions suitable to kill non-transformed plants, retain viability in transformed plants. For instance by growing the plants in a medium or soil that contains certain chemicals, such that only those plants expressing the transgenes can survive. In one particular embodiment, after obtaining a transgenic plant that appear to be expressing the transgene, a functional screening may be carried out by growing the plants under water deficit conditions to select for those that can tolerate such a condition.

In another aspect, the present disclosure provides a kit for generating a transgenic plant having an altered stress response or an altered phenotype compared to an unmodified plant, characterized in that the kit comprises: an expression construct including a DNA sequence selected from at least one of the DRGs disclosed herein, said DNA sequence may be operatively linked to an promoter suitable for expressing said DNA sequence in a plant cell.

Preferably the kit further includes targeting means for targeting the activity of the protein expressed from the construct to certain tissues or cells of the plant. Preferably the targeting means comprises an inducible, tissue-specific promoter for specific expression of the DNA sequence within certain tissues of the plant. Alternatively the targeting means may be a signal sequence encoded by said expression construct and may contain a series of amino acids covalently linked to the expressed protein.

In accordance with the kit of the present invention, the DNA sequence may encode a peptide having at least 70%, more preferably at least 90%, more preferably at least 98%, or even 99% identity to the peptide encoded by coding sequences selected from at least one of the DRGs disclosed herein. In one aspect, said DNA sequence may be oriented in an antisense direction relative to said promoter within said construct.

### BRIEF DESCRIPTION OF THE DRAWINGS

FIG. 1 summarizes the classification of drought responsive transcripts in soybean leaf tissues based on reported or predicted function of the corresponding proteins.

FIG. 2 summarizes the classification of drought responsive transcripts in soybean root tissues based on reported or predicted function of the corresponding proteins.

FIG. 3 lists all sequences of all validated transcripts listed in Table 5.

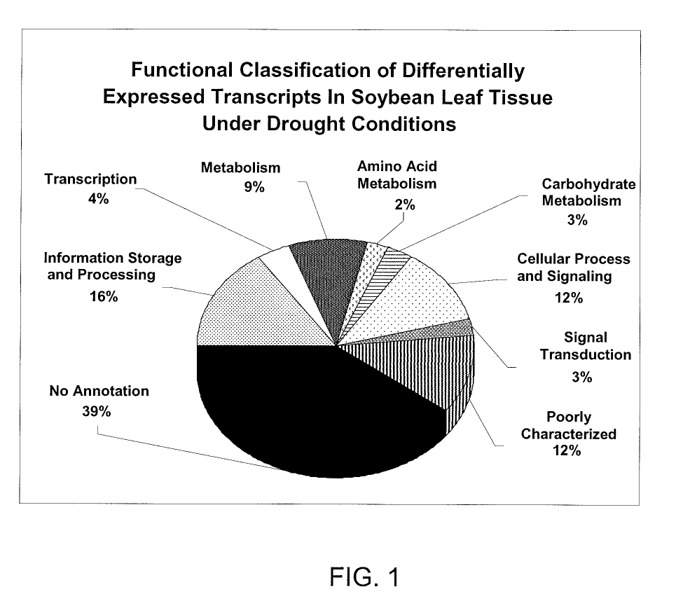

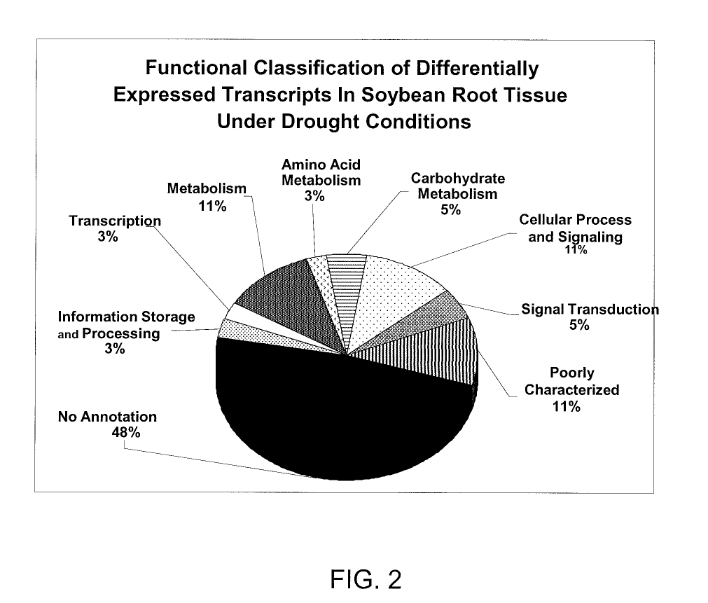

### DETAILED DESCRIPTION

The methods and materials described herein relate to gene expression profiling using microarrays, and follow-up analysis to decode the regulatory network that controls a plant's response to stress. More particularly, drought response is analyzed at the molecular level to identify genes and/or promoters which may be activated under water deficit conditions. The coding sequences of such genes may be introduced into a host plant to obtain transgenic plants that are more tolerant to drought than unmodified plants.

It is to be understood that the materials and methods are taught by way of example, and not by limitation. The disclosed instrumentalities may be broader than the particular methods and materials described herein, which may vary within the skill of the art. It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the related art. The following terminology and grammatical variants are used in accordance with the definitions set out below.

The present disclosure provides genes whose expression levels are altered in response to stress conditions in soybean plants using genome-wide microarray (or gene chip) analysis of soybean plants grown under water deficit conditions. Those genes identified using microarray analysis may be subject to validation to confirm that their expression levels are altered under the stress conditions. Validation may be conducted using high throughput two-step qRT-PCR or by the delta delta CT method.

Sequences of those genes that have been validated may be subject to further sequence analysis by comparing their sequences to published sequences of various families of genes or proteins. For instance, some of these DRGs may encode proteins with substantial sequence similarity to known transcription factors. These transcription factors may play a role in the stress response by activating the transcription of other genes.

The present disclosure provides a system and a method for expressing a protein that may enhance a host's capability to grow or to survive in an adverse environment characterized by water deficit. Although plants are the most preferred host for purpose of this disclosure, the genetic constructs described herein may be introduced into other eukaryotic organisms, if the traits conferred upon these organisms by the constructs are desirable.

The term “transgenic plant” refers to a host plant into which a gene construct has been introduced. A gene construct, also referred to as a construct, an expression construct, or a DNA construct, generally contains as its components at least a coding sequence and a regulatory sequence. A gene construct typically contains at least on component that is foreign to the host plant. For purpose of this disclosure, all components of a gene construct may be from the host plant, but these components are not arranged in the host in the same manner as they are in the gene construct. A regulatory sequence is a non-coding sequence that typically contribute to the regulation of gene expression, at the transcription or translation levels. It is to be understood that certain segments in the coding sequence may be translated but may be later removed from the functional protein. An example of these segments is the so-called signal peptide, which may facilitate the maturation or localization of the translated protein, but is typically removed once the protein reaches its destination. Examples of a regulatory sequence include but are not limited to a promoter, an enhancer, and certain post-transcriptional regulatory elements.

After its introduction into a host plant, a gene construct may exist separately from the host chromosomes. Preferably, the entire gene construct, or at least part of it, is integrated onto a host chromosome. The integration may be mediated by a recombination event, which may be homologous, or non-homologous recombination. The term “express” or “expression” refers to production of RNAs using DNAs as template through transcription or translation of proteins from RNAs or the combination of both transcription and translation.

A “host cell,” as used herein, refers to a prokaryotic or eukaryotic cell that contains heterologous DNA which has been introduced into the cell by any means, e.g., electroporation, calcium phosphate precipitation, microinjection, transformation, viral infection, and/or the like. A “host plant” is a plant into which a transgene is to be introduced.

A “vector” is a composition for facilitating introduction, replication and/or expression of a selected nucleic acid in a cell. Vectors include, for example, plasmids, cosmids, viruses, yeast artificial chromosomes (YACs), etc. A “vector nucleic acid” is a nucleic acid vector into which heterologous nucleic acid is optionally inserted and which can then be introduced into an appropriate host cell. Vectors preferably have one or more origins of replication, and one or more sites into which the recombinant DNA can be inserted. Vectors often have convenient markers by which cells with vectors can be selected from those without. By way of example, a vector may encode a drug resistance gene to facilitate selection of cells that are transformed with the vector. Common vectors include plasmids, phages and other viruses, and “artificial chromosomes.” “Expression vectors” are vectors that comprise elements that provide for or facilitate transcription of nucleic acids which are cloned into the vectors. Such elements may include, for example, promoters and/or enhancers operably coupled to a nucleic acid of interest.

“Plasmids” generally are designated herein by a lower case “p” preceded and/or followed by capital letters and/or numbers, in accordance with standard nomenclatures that are familiar to those of skill in the art. Starting plasmids disclosed herein are either commercially available, publicly available on an unrestricted basis, or can be constructed from available plasmids by routine application of well known, published procedures. Many plasmids and other cloning and expression vectors are well known and readily available to those of skill in the art. Moreover, those of skill readily may construct any number of other plasmids suitable for use as described below. The properties, construction and use of such plasmids, as well as other vectors, is readily apparent to those of ordinary skill upon reading the present disclosure.

When a molecule is identified in or can be isolated from a organism, it can be said that such a molecule is derived from said organism. When two organisms have significant difference in the genetic materials in their respective genomes, these two organisms can be said to be genetically different. For purpose of this disclosure, the term “plant” means a whole plant, a seed, or any organ or tissue of a plant that may potentially develop into a whole plant.

The term “isolated” means that the material is removed from its original environment, such as the native or natural environment if the material is naturally occurring. For example, a naturally-occurring nucleic acid, polypeptide, or cell present in a living animal is not isolated, but the same polynucleotide, polypeptide, or cell separated from some or all of the coexisting materials in the natural system, is isolated, even if subsequently reintroduced into the natural system. Such nucleic acids can be part of a vector and/or such nucleic acids or polypeptides could be part of a composition, and still be isolated in that such vector or composition is not part of its natural environment.

A “recombinant nucleic acid” is one that is made by recombining nucleic acids, e.g., during cloning, DNA evolution or other procedures. A “recombinant polypeptide” is a polypeptide which is produced by expression of a recombinant nucleic acid. An “amino acid sequence” is a polymer of amino acid residues (a protein, polypeptide, etc.) or a character string representing an amino acid polymer, depending on context. Either the given nucleic acid or the complementary nucleic acid can be determined from any specified polynucleotide sequence.

The terms “nucleic acid,” or “polynucleotide” refer to a deoxyribonucleotide, in the case of DNA, or ribonucleotide in the case of RNA polymer in either single- or double-stranded form, and unless otherwise specified, encompasses known analogues of natural nucleotides that can be incorporated into nucleic acids in a manner similar to naturally occurring nucleotides. A “polynucleotide sequence” is a nucleic acid which is a polymer of nucleotides (A, C, T, U, G, etc. or naturally occurring or artificial nucleotide analogues) or a character string representing a nucleic acid, depending on context. Either the given nucleic acid or the complementary nucleic acid can be determined from any specified polynucleotide sequence.

A “subsequence” or “fragment” is any portion of an entire sequence of a DNA, RNA or polypeptide molecule, up to and including the complete sequence. Typically a subsequence or fragment comprises less than the full-length sequence, and is sometimes referred to as the “truncated version.”

Nucleic acids and/or nucleic acid sequences are “homologous” when they are derived, naturally or artificially, from a common ancestral nucleic acid or nucleic acid sequence. Proteins and/or protein sequences are homologous when their encoding DNAs are derived, naturally or artificially, from a common ancestral nucleic acid or nucleic acid sequence. Similarly, nucleic acids and/or nucleic acid sequences are homologous when they are derived, naturally or artificially, from a common ancestral nucleic acid or nucleic acid sequence. The homologous molecules can be termed homologs. For example, any naturally occurring DRGs, as described herein, can be modified by any available mutagenesis method. When expressed, this mutagenized nucleic acid encodes a polypeptide that is homologous to the protein encoded by the original DRGs. Homology is generally inferred from sequence identity between two or more nucleic acids or proteins (or sequences thereof). The precise percentage of identity between sequences that is useful in establishing homology varies with the nucleic acid and protein at issue, but as little as 25% sequence identity is routinely used to establish homology. Higher levels of sequence identity, e.g., 30%, 40%, 50%, 60%, 70%, 80%, 90%, 95% or 99% or more can also be used to establish homology. Methods for determining sequence identity percentages (e.g., BLASTP and BLASTN using default parameters) are described herein and are generally available.

The terms “identical” or “sequence identity” in the context of two nucleic acid sequences or amino acid sequences of polypeptides refers to the residues in the two sequences which are the same when aligned for maximum correspondence over a specified comparison window. A “comparison window”, as used herein, refers to a segment of at least about 20 contiguous positions, usually about 50 to about 200, more usually about 100 to about 150 in which a sequence may be compared to a reference sequence of the same number of contiguous positions after the two sequences are aligned optimally. Methods of alignment of sequences for comparison are well-known in the art. Optimal alignment of sequences for comparison may be conducted by the local homology algorithm of Smith and Waterman (1981) Adv. Appl. Math. 2:482; by the alignment algorithm of Needleman and Wunsch (1970) J. Mol. Biol. 48:443; by the search for similarity method of Pearson and Lipman (1988) Proc. Nat. Acad. Sci. U.S.A. 85:2444; by computerized implementations of these algorithms (including, but not limited to CLUSTAL in the PC/Gene program by Intelligentics, Mountain View Calif., GAP, BESTFIT, BLAST, FASTA, and TFASTA in the Wisconsin Genetics Software Package, Genetics Computer Group (GCG), 575 Science Dr., Madison, Wis., U.S.A.); the CLUSTAL program is well described by Higgins and Sharp (1988) Gene 73:237-244 and Higgins and Sharp (1989) CABIOS 5:151-153; Corpet et al. (1988) Nucleic Acids Res. 16:10881-10890; Huang et al (1992) Computer Applications in the Biosciences 8:155-165; and Pearson et al. (1994) Methods in Molecular Biology 24:307-331. Alignment is also often performed by inspection and manual alignment.

In one class of embodiments, the polypeptides herein are at least 70%, generally at least 75%, optionally at least 80%, 85%, 90%, 98% or 99% or more identical to a reference polypeptide, e.g., those that are encoded by DNA sequences as set forth by any one of the DRGs disclosed herein or a fragment thereof, e.g., as measured by BLASTP (or CLUSTAL, or any other available alignment software) using default parameters. Similarly, nucleic acids can also be described with reference to a starting nucleic acid, e.g., they can be 50%, 60%, 70%, 75%, 80%, 85%, 90%, 98%, 99% or more identical to a reference nucleic acid, e.g., those that are set forth by any one of the DRGs disclosed herein or a fragment thereof, e.g., as measured by BLASTN (or CLUSTAL, or any other available alignment software) using default parameters. When one molecule is said to have certain percentage of sequence identity with a larger molecule, it means that when the two molecules are optimally aligned, said percentage of residues in the smaller molecule finds a match residue in the larger molecule in accordance with the order by which the two molecules are optimally aligned.

The term “substantially identical” as applied to nucleic acid or amino acid sequences means that a nucleic acid or amino acid sequence comprises a sequence that has at least 90% sequence identity or more, preferably at least 95%, more preferably at least 98% and most preferably at least 99%, compared to a reference sequence using the programs described above (preferably BLAST) using standard parameters. For example, the BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1989)). Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

The term “polypeptide” is used interchangeably with the terms “polypeptides” and “protein(s)”, and refers to a polymer of amino acid residues. A ‘mature protein’ is a protein which is full-length and which, optionally, includes glycosylation or other modifications typical for the protein in a given cellular environment.

The term “variant” or “mutant” with respect to a polypeptide refers to an amino acid sequence that is altered by one or more amino acids with respect to a reference sequence. The variant may have “conservative” changes, wherein a substituted amino acid has similar structural or chemical properties, e.g., replacement of leucine with isoleucine. Alternatively, a variant may have “nonconservative” changes, e.g., replacement of a glycine with a tryptophan. Analogous minor variation can also include amino acid deletion or insertion, or both. Guidance in determining which amino acid residues can be substituted, inserted, or deleted without eliminating biological or immunological activity can be found using computer programs well known in the art, for example, DNASTAR software.

A variety of additional terms are defined or otherwise characterized herein. In practicing the instrumentalities described herein, many conventional techniques in molecular biology, microbiology, and recombinant DNA are optionally used. These techniques are well known to those of ordinary skill in the art. For example, one skilled in the art would be familiar with techniques for in vitro amplification methods, including the polymerase chain reaction (PCR), for the production of the homologous nucleic acids described herein.

In addition, commercially available kits may facilitate the purification of plasmids or other relevant nucleic acids from cells. See, for example, EasyPrep™ and FlexiPrep™ kits, both from Pharmacia Biotech; StrataClean™ from Stratagene; and, QIAprep™ from Qiagen. Any isolated and/or purified nucleic acid can be further manipulated to produce other nucleic acids, used to transfect cells, incorporated into related vectors to infect organisms, or the like. Typical cloning vectors contain transcription terminators, transcription initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or both.

Various types of mutagenesis are optionally used to modify DRGs and their encoded polypeptides, as described herein, to produce conservative or non-conservative variants. Any available mutagenesis procedure can be used. Such mutagenesis procedures optionally include selection of mutant nucleic acids and polypeptides for one or more activity of interest. Procedures that can be used include, but are not limited to: site-directed point mutagenesis, random point mutagenesis, in vitro or in vivo homologous recombination (DNA shuffling), mutagenesis using uracil-containing templates, oligonucleotide-directed mutagenesis, phosphorothioate-modified DNA mutagenesis, mutagenesis using gapped duplex DNA, point mismatch repair, mutagenesis using repair-deficient host strains, restriction-selection and restriction-purification, deletion mutagenesis, mutagenesis by total gene synthesis, double-strand break repair, mutagenesis by chimeric constructs, and many others known to persons of skill in the art.

In one embodiment, mutagenesis can be guided by known information about the naturally occurring molecule or altered or mutated naturally occurring molecule. By way of example, this known information may include sequence, sequence comparisons, physical properties, crystal structure and the like. In another class of mutagenesis, modification is essentially random, e.g., as in classical DNA shuffling.

Polypeptides may include variants, in which the amino acid sequence has at least 70% identity, preferably at least 80% identity, typically 90% identity, preferably at least 95% identity, more preferably at least 98% identity and most preferably at least 99% identity, to the amino acid sequences as encoded by the DNA sequences set forth in any one of the DRGs disclosed herein.

The aforementioned polypeptides may be obtained by any of a variety of methods. Smaller peptides (less than 50 amino acids long) are conveniently synthesized by standard chemical techniques and can be chemically or enzymatically ligated to form larger polypeptides. Polypeptides can be purified from biological sources by methods well known in the art, for example, as described in Protein Purification, Principles and Practice, Second Edition Scopes, Springer Verlag, N.Y. (1987) Polypeptides are optionally but preferably produced in their naturally occurring, truncated, or fusion protein forms by recombinant DNA technology using techniques well known in the art. These methods include, for example, in vitro recombinant DNA techniques, synthetic techniques and in vivo genetic recombination. See, for example, the techniques described in Sambrook et al. (2001) Molecular Cloning, A Laboratory Manual, Third Edition, Cold Spring Harbor Press, N.Y.; and Ausubel et al., eds. (1997) Current Protocols in Molecular Biology, Green Publishing Associates, Inc., and John Wiley & Sons, Inc., N.Y (supplemented through 2002). RNA encoding the proteins may also be chemically synthesized. See, for example, the techniques described in Oligonucleotide Synthesis, (1984) Gait ed., IRL Press, Oxford, which is incorporated by reference herein in its entirety.

The nucleic acid molecules described herein may be expressed in a suitable host cell or an organism to produce proteins. Expression may be achieved by placing a nucleotide sequence encoding these proteins into an appropriate expression vector and introducing the expression vector into a suitable host cell, culturing the transformed host cell under conditions suitable for expression of the proteins described or variants thereof, or a polypeptide that comprises one or more domains of such proteins. The recombinant proteins from the host cell may be purified to obtain purified and, preferably, active protein. Alternatively, the expressed protein may be allowed to function in the intact host cell or host organism.

Appropriate expression vectors are known in the art, and may be purchased or applied for use according to the manufacturer's instructions to incorporate suitable genetic modifications. For example, pET-14b, pcDNA1Amp, and pVL1392 are available from Novagen and Invitrogen, and are suitable vectors for expression in E. coli, mammalian cells and insect cells, respectively. These vectors are illustrative of those that are known in the art, and many other vectors can be used for the same purposes. Suitable host cells can be any cell capable of growth in a suitable media and allowing purification of the expressed protein. Examples of suitable host cells include bacterial cells, such as E. coli, Streptococci, Staphylococci, Streptomyces and Bacillus subtilis cells; fungal cells such as Saccharomyces and Aspergillus cells; insect cells such as Drosophila S2 and Spodoptera Sf9 cells, mammalian cells such as CHO, COS, HeLa, 293 cells; and plant cells.

Culturing and growth of the transformed host cells can occur under conditions that are known in the art. The conditions will generally depend upon the host cell and the type of vector used. Suitable culturing conditions may be used such as temperature and chemicals and will depend on the type of promoter utilized.

Purification of the proteins or domains of such proteins, if desired, may be accomplished using known techniques without performing undue experimentation. Generally, the transformed cells expressing one of these proteins are broken, crude purification occurs to remove debris and some contaminating proteins, followed by chromatography to further purify the protein to the desired level of purity. Host cells may be broken by known techniques such as homogenization, sonication, detergent lysis and freeze-thaw techniques. Crude purification can occur using ammonium sulfate precipitation, centrifugation or other known techniques. Suitable chromatography includes anion exchange, cation exchange, high performance liquid chromatography (HPLC), gel filtration, affinity chromatography, hydrophobic interaction chromatography, etc. Well known techniques for refolding proteins can be used to obtain the active conformation of the protein when the protein is denatured during intracellular synthesis, isolation or purification.

In general, DRG proteins or domains, or antibodies to such proteins can be purified, either partially (e.g., achieving a 5×, 10×, 10×, 500×, or 1000× or greater purification), or even substantially to homogeneity (e.g., where the protein is the main component of a solution, typically excluding the solvent (e.g., water or DMSO) and buffer components (e.g., salts and stabilizers) that the protein is suspended in, e.g., if the protein is in a liquid phase), according to standard procedures known to and used by those of skill in the art. Accordingly, the polypeptides can be recovered and purified by any of a number of methods well known in the art, including, e.g., ammonium sulfate or ethanol precipitation, acid or base extraction, column chromatography, affinity column chromatography, anion or cation exchange chromatography, phosphocellulose chromatography, hydrophobic interaction chromatography, hydroxylapatite chromatography, lectin chromatography, gel electrophoresis and the like. Protein refolding steps can be used, as desired, in making correctly folded mature proteins. High performance liquid chromatography (HPLC), affinity chromatography or other suitable methods can be employed in final purification steps where high purity is desired. In one embodiment, antibodies made against the proteins described herein are used as purification reagents, e.g., for affinity-based purification of proteins comprising one or more DRG protein domains or antibodies thereto. Once purified, partially or to homogeneity, as desired, the polypeptides are optionally used e.g., as assay components, therapeutic reagents or as immunogens for antibody production.

In addition to other references noted herein, a variety of purification methods are well known in the art, including, for example, those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana, Bioseparation of Proteins, Academic Press, Inc. (1997); Bollag et al., Protein Methods, 2nd Edition Wiley-Liss, NY; Walker (1996) The Protein Protocols Handbook Humana Press, NJ; Harris and Angal Protein Purification Applications: A Practical Approach IRL Press at Oxford, Oxford, England (1990); Scopes, Protein Purification: Principles and Practice 3rd Edition Springer Verlag, NY (1993); Janson and Ryden, Protein Purification: Principles, High Resolution Methods and Applications, Second Edition Wiley-VCH, NY (1998); and Walker, Protein Protocols on CD-ROM Humana Press, NJ (1998); and the references cited therein.

After synthesis, expression and/or purification, proteins may possess a conformation different from the desired conformations of the relevant polypeptides. For example, polypeptides produced by prokaryotic systems often are optimized by exposure to chaotropic agents to achieve proper folding. During purification from, e.g., lysates derived from E. coli, the expressed protein is optionally denatured and then renatured. This is accomplished, e.g., by solubilizing the proteins in a chaotropic agent such as guanidine HCl. In general, it is occasionally desirable to denature and reduce expressed polypeptides and then to cause the polypeptides to re-fold into the preferred conformation. For example, guanidine, urea, DTT, DTE, and/or a chaperonin can be added to a translation product of interest. Methods of reducing, denaturing and renaturing proteins are well known to those of skill in the art. Debinski, et al., for example, describe the denaturation and reduction of inclusion body proteins in guanidine-DTE. The proteins can be refolded in a redox buffer containing, e.g., oxidized glutathione and L-arginine. Refolding reagents can be flowed or otherwise moved into contact with the one or more polypeptide or other expression product, or vice-versa.

In another aspect, antibodies to the DRG proteins or fragments thereof may be generated using methods that are well known in the art. The antibodies may be utilized for detecting and/or purifying the DRG proteins, optionally discriminating the proteins from various homologues. As used herein, the term “antibody” includes, but is not limited to, polyclonal antibodies, monoclonal antibodies, humanized or chimeric antibodies and biologically functional antibody fragments, which are those fragments sufficient for binding of the antibody fragment to the protein.

General protocols that may be adapted for detecting and measuring the expression of the described DRG proteins using the above mentioned antibodies are known. Such methods include, but are not limited to, dot blotting, western blotting, competitive and noncompetitive protein binding assays, enzyme-linked immunosorbant assays (ELISA), immunohistochemistry, fluorescence-activated cell sorting (FACS), and other protocols that are commonly used and widely described in scientific and patent literature.

Sequence of the DRG genes may also be used in genetic mapping of plants or in plant breeding. Polynucleotides derived from the DRG gene sequences may be used in in situ hybridization to determine the chromosomal locus of the DRG genes on the chromosomes. These polynucleotides may also be used to detect segregation of different alleles at certain DRG loci.

Sequence information of the DRG genes may also be used to design oligonucleotides for detecting DRG mRNA levels in the cells or in plant tissues. For example, the oligonucleotides can be used in a Northern blot analysis to quantify the levels of DRG mRNA. Moreover, full-length or fragment of the DRG genes may be used in preparing microarrays (or gene chips). Full-length or fragment of the DRG genes may also be used in microarray experiments to study expression profile of the DRG genes. High-throughput screening can be conducted to measure expression levels of the DRG genes in different cells or tissues. Various compounds or other external factors may be screened for their effects expression of the DRG gene expression.

Sequences of the DRG genes and proteins may also provide a tool for identification of other proteins that may be involved in plant drought response. For example, chimeric DRG proteins can be used as a “bait” to identify other proteins that interact with DRG proteins in a yeast two-hybrid screening. Recombinant DRG proteins can also be used in pull-down experiment to identify their interacting proteins. These other proteins may be cofactors that enhance the function of the DRG proteins, or they may be DRG proteins themselves which have not been identified in the experiments disclosed herein.

The DRG polypeptides may possess structural features which can be recognized, for example, by using immunological assays. The generation of antisera which specifically bind the DRG polypeptides, as well as the polypeptides which are bound by such antisera, are a feature of the disclosed embodiments.

In order to produce antisera for use in an immunoassay, one or more of the immunogenic DRG polypeptides or fragments thereof are produced and purified as described herein. For example, recombinant protein may be produced in a host cell such as a bacterial or an insect cell. The resultant proteins can be used to immunize a host organism in combination with a standard adjuvant, such as Freund's adjuvant. Commonly used host organisms include rabbits, mice, rats, donkeys, chickens, goats, horses, etc. An inbred strain of mice may also be used to obtain more reproducible results due to the virtual genetic identity of the mice. The mice are immunized with the immunogenic DRG polypeptides in combination with a standard adjuvant, such as Freund's adjuvant, and a standard mouse immunization protocol. See, for example, Harlow and Lane, Antibodies, A Laboratory Manual, Cold Spring Harbor Publications, New York (1988), which provides comprehensive descriptions of antibody generation, immunoassay formats and conditions that can be used to determine specific immunoreactivity. Alternatively, one or more synthetic or recombinant DRG polypeptides or fragments thereof derived from the sequences disclosed herein is conjugated to a carrier protein and used as an immunogen.

Antisera that specifically bind the DRG proteins may be used in a range of applications, including but not limited to immunofluorescence staining of cells for the expression level and localization of the DRG proteins, cytological staining for the expression of DRG proteins in tissues, as well as in Western blot analysis.

Another aspect of the disclosure includes screening for potential or candidate modulators of DRG protein activity. For example, potential modulators may include small molecules, organic molecules, inorganic molecules, proteins, hormones, transcription factors, or the like, which can be contacted to a cell or certain tissues that express the DRG proteins to assess the effects, if any, of the candidate modulator upon DRG protein activity.

Alternatively, candidate modulators may be screened to modulate expression of DRG proteins. For example, potential modulators may include small molecules, organic molecules, inorganic molecules, proteins, hormones, transcription factors, or the like, which can be contacted to a cell or certain tissues that express the DRG proteins, to assess the effects, if any, of the candidate modulator upon DRG protein expression. Expression of a DRG gene described herein may be detected, for example, via Northern blot analysis or quantitative (optionally real time) RT-PCR, before and after application of potential expression modulators. Alternatively, promoter regions of the various DRG genes may be coupled to reporter constructs including, without limitation, CAT, beta-galactosidase, luciferase or any other available reporter, and may similarly be tested for expression activity modulation by the candidate modulator. Promoter regions of the various genes are generally sequences in the proximity upstream of the start site of transcription, typically within 1 Kb or less of the start site, such as within 500 bp, 250 bp or 100 bp of the start site. In certain cases, a promoter region may be located between 1 and 5 Kb from the start site.

In either case, whether the assay is to detect modulated activity or expression, a plurality of assays may be performed in a high-throughput fashion, for example, using automated fluid handling and/or detection systems in serial or parallel fashion. Similarly, candidate modulators can be tested by contacting a potential modulator to an appropriate cell using any of the activity detection methods herein, regardless of whether the activity that is detected is the result of activity modulation, expression modulation or both.

A method of modifying a plant may include introducing into a host plant one or more DRG genes described above. The DRG genes may be placed in an expression construct, which may be designed such that the DRG protein(s) are expressed constitutively, or inducibly. The construct may also be designed such that the DRG protein(s) are expressed in certain tissue(s), but not in other tissue(s). The DRG protein(s) may enhance the ability of the host plant in drought tolerance, such as by reducing water loss or by other mechanisms that help a plant cope with water deficit growth conditions. The host plant may include any plants whose growth and/or yield may be enhanced by a modified drought response. Methods for generating such transgenic plants is well known in the field. See e.g., Leandro Peña (Editor), Transgenic Plants: Methods and Protocols (Methods in Molecular Biology), Humana Press, 2004.

The use of gene inhibition technologies such as antisense RNA or co-suppression or double stranded RNA interference is also within the scope of the present disclosure. In these approaches, the isolated gene sequence is operably linked to a suitable regulatory element. In one embodiment of the disclosure, the construct contains a DNA expression cassette that contains, in addition to the DNA sequences required for transformation and selection in said cells, a DNA sequence that encodes a DRG proteins or a DRG modulator protein, with at least a portion of said DNA sequence in an antisense orientation relative to the normal presentation to the transcriptional regulatory region, operably linked to a suitable transcriptional regulatory region such that said recombinant DNA construct expresses an antisense RNA or portion thereof of an antisense RNA in the resultant transgenic plant.

It is apparent to one of skill in the art that the polynucleotide encoding the DRG proteins or a DRG modulator proteins can be in the antisense (for inhibition by antisense RNA) or sense (for inhibition by co-suppression) orientation, relative to the transcriptional regulatory region. Alternatively a combination of sense and antisense RNA expression can be utilized to induce double stranded RNA interference. See, e.g., Chuang and Meyerowitz, PNAS 97: 4985-4990, 2000; also Smith et al., Nature 407: 319-320, 2000.

These methods for generation of transgenic plants generally entail the use of transformation techniques to introduce the gene or construct encoding the DRG proteins or a DRG modulator proteins, or a part or a homolog thereof, into plant cells. Transformation of a plant cell can be accomplished by a variety of different methodology. Methods that have general utility include, for example, Agrobacterium based systems, using either binary and/or cointegrate plasmids of both A. tumifaciens and A. rhyzogenies, (See e.g., U.S. Pat. No. 4,940,838, U.S. Pat. No. 5,464,763), the biolistic approach (See e.g., U.S. Pat. No. 4,945,050, U.S. Pat. No. 5,015,580, U.S. Pat. No. 5,149,655), microinjection, (See e.g., U.S. Pat. No. 4,743,548), direct DNA uptake by protoplasts, (See e.g., U.S. Pat. No. 5,231,019, U.S. Pat. No. 5,453,367) or needle-like whiskers (See e.g., U.S. Pat. No. 5,302,523). Any method for the introduction of foreign DNA into a plant cell and for expression therein may be used within the context of the present disclosure.

Plants that are capable of being transformed encompass a wide range of species, including but not limited to soybean, corn, potato, rice, wheat and many other crops, fruit plants, vegetables and tobacco. See generally, Vain, P., Thirty years of plant transformation technology development, Plant Biotechnol J. 2007 March; 5(2):221-9. Any plants that are capable of taking in foreign DNA and transcribing the DNA into RNA and/or further translating the RNA into a protein may be a suitable host.

The modulators described above that may alter the expression levels or the activity of the DRG proteins (collectively called DRG modulators) may also be introduced into a host plant in the same or similar manner as described above.

The DRG proteins or the DRG modulators may be used to modify a target plant by causing them to be assimilated by the plant. Alternatively, the DRG proteins or the DRG modulators may be applied to a target plant by causing them to be in contact with the plant, or with a specific organ or tissue of the plant. In one embodiment, organic or inorganic molecules that can function as DRG modulators may be caused to be in contact with a plant such that these chemicals may enhance the drought response of the target plant.

In addition to the DRG modulators, DRG polypeptides or DRG nucleic acids, a composition containing other ingredients may be introduced, administered or delivered to the plant to be modified. In one aspect, a composition containing an agriculturally acceptable ingredient may be used in conjunction with the DRG modulators to be administered or delivered to the plant.

Bioinformatic systems are widely used in the art, and can be utilized to identify homology or similarity between different character strings, or can be used to perform other desirable functions such as to control output files, provide the basis for making presentations of information including the sequences and the like. Examples include BLAST, discussed supra. For example, commercially available databases, computers, computer readable media and systems may contain character strings corresponding to the sequence information herein for the DRG polypeptides and nucleic acids described herein. These sequences may include specifically the DRG sequences listed herein and the various silent substitutions and conservative substitutions thereof.

The bioinformatic systems contain a wide variety of information that includes, for example, a complete sequence listings for the entire genome of an individual organism representing a species. Thus, for example, using the DRG sequences as a basis for comparison, the bioinformatic systems may be used to compare different types of homology and similarity of various stringency and length on the basis of reported data. These comparisons are useful to identify homologs or orthologs where, for example, the basic DRG gene ortholog is shown to be conserved across different organisms. Thus, the bioinformatic systems may be used to detect or recognize the homologs or orthologs, and to predict the function of recognized homologs or orthologs. By way of example, many homology determination methods have been designed for comparative analysis of sequences of biopolymers including nucleic acids, proteins, etc. With an understanding of hydrogen bonding between the principal bases in natural polynucleotides, models that simulate annealing of complementary homologous polynucleotide strings can also be used as a foundation of sequence alignment or other operations typically performed on the character strings corresponding to the sequences herein. One example of a software package for calculating sequence similarity is BLAST, which can be adapted to the present invention by inputting character strings corresponding to the sequences herein.

The software can also include output elements for controlling nucleic acid synthesis (e.g., based upon a sequence or an alignment of a sequences herein) or other operations which occur downstream from an alignment or other operation performed using a character string corresponding to a sequence herein.

In an additional aspect, kits may embody any of the methods, compositions, systems or apparatus described above. Kits may optionally comprise one or more of the following: (1) a composition, system, or system component as described herein; (2) instructions for practicing the methods described herein, and/or for using the compositions or operating the system or system components herein; (3) a container for holding components or compositions, and, (4) packaging materials.

### EXAMPLES

The nonlimiting examples that follow report general procedures, reagents and characterization methods that teach by way of example, and should not be construed in a narrowing manner that limits the disclosure to what is specifically disclosed. Those skilled in the art will understand that numerous modifications may be made and still the result will fall within the spirit and scope of the present invention.

### Example 1

### Gene Profiling of Drought Response Genes in Soybean

Genetic material and the growing system: We have used cv Williams 82 for our green house experiments. Plants were grown in Turface-sand medium in 3 gallon pots. One-month old soybean plants were subjected to gradual stress by withholding water and the samples were collected in three biological replicates. To quantitate the stress level we monitored relative water content (RWC), leaf water potential, and turface-soil mixture water potential and moisture content. Leaf RWC, leaf water potential, and soil water content were 95%. −0.3 MPa, and 20% (v/v), respectively, for well-watered samples. These values were 65%, −1.6 MPa, 9.6% for the water-stressed samples.

RNA isolation and the microarray: Flash-frozen plant tissue samples were ground under liquid nitrogen with a mortar and pestle. Total RNA is extracted using a modified Trizol (Invitrogen Corp., Carlsbad, Calif.) protocol followed by additional purification using RNEasy columns (Qiagen, Valencia, Calif.). RNA quality is assayed using an Agilent 2100Bioanalyzer to determine integrity and purity; RNA purity is further assayed by measuring absorbance at 200 nm and 280 nm using a NanoDrop spectrophotometer.

Microarray hybridization, data acquisition, and image processing: We used the pair wise comparison experimental plan for the microarray experiments. A total number of 12 hybridizations were conducted as: 2 biological conditions×3 biological replicates×2 tissue types. First strand GDNA were synthesized with 30 pg total RNA and T7-Oligo(dT) primer. The total RNA were processed to use on Affymetrix Soybean GeneChip arrays, according to the manufacturer's protocol (Affymetrix, Santa Clara, Calif.). The GeneChip soybean genome array consists of 35,611 soybean transcripts (details as in the results description). Microarray hybridization, washing and scanning with Affymetrix high density scanner were performed according to the standard protocols. The scanned images were processed and the data acquired using GCOS. Having selected genes that are significantly correlated with phenotype or treatment, data mining is conducted using a variety of tools focusing on class discovery and class comparison in order to identify and prioritize candidates.

Confirmation of gene expression by qRT-PCR: Validation of the microarray profiling and the expression of significant genes at significant time points in the experiments were determined by a high-throughput two-step quantitative RT-PCR (qRT-PCR) assay using SYBR Green on the ABI 7900 HT and by the delta delta CT method (Applied Biosystems) developed in course of these studies.

One-month old soybean plants were subjected to gradual stress by withholding water and the samples were collected in three biological replicates. To quantitate the stress level we monitored relative water content (RWC), leaf water potential, and surface-soil mixture water potential and moisture content. Total RNA isolation and microarray hybridizations were conducted using standard protocols. We used 60K soybean Affymetrix GeneChips for the transcriptome profiling. The GeneChip® Soybean Genome Array is a 49-format, 11-micron array design, and it contains 11 probe pairs per probe set. Sequence Information for this array includes public content from GenBank® and dbEST. Sequence clusters were created from UniGene Build 13 (Nov. 5, 2003). The GeneChip® Soybean Genome Array contains ˜60,000 transcripts and 37,500 transcripts are specific for soybean. In addition to extensive soybean coverage, the GeneChip® Soybean Genome Array includes probe sets to detect approximately 15,800 transcripts for Phytophthora sojae (a water mold that commonly attacks soybean crops) as well as 7,500 Heterodera glycines (cyst nematode pathogen) transcripts. (www.affymetrix.com) The affymetrix chip hybridization data of the soybean root under stress were processed. The statistical analysis of the data was performed using the mixed linear model ANOVA (log2 (pm)˜probe+trt+array (trt)). The response variable “log2 (pm)” is the log base 2 transformed perfect match intensity after RMA background correction and quantile normalization; the covarlate “probe” indicates the probe levels since for each gene there are usually 11 probes; “tit” is the treatment/condition effect and it specifies if the array considered is treatment or control; “array(trt)” is the array nested within trt effect, as there are replicate arrays for each treatment.

FDR adjusted p-value is less than 0.01 cutoff point where fdr_p is less than 0.01.

The statistically analyzed data were sorted and the functional classifications (KOG and GO) were performed. Significantly differentially expressed transcripts in root and leaf tissues between well-watered and water stressed condition are:

p value adjusted FDR 5%
Leaf tissue—2497 up regulated, 938 down regulated
Root tissue—885 up regulated, 5428 down regulated
Leaf vs root—769 up regulated, 406 down regulated
   p value adjusted FDR 1%
Leaf tissue—2088 up regulated, 863 down regulated
Root tissue—800 up regulated, 5428 down regulated
Leaf vs root—576 up regulated, 211 down regulated

The functional classification of the differentially expressed genes in soybean leaf under drought condition is summarized in Table 1, which shows the numbers of genes that are either up- or down-regulated in each category as defined by protein function.

TABLE 1
Functional Classification of drought responsive transcripts in
soybean leaf tissues:

| | | | Up + |
|---|---|---|---|
| | Up | Down | Down |
| Leaf tissue | regulated | regulated | regulated |
| Information Storage and Processing | 508 | 29 | 537 |
| Transcription | 106 | 27 | 133 |
| Metabolism | 225 | 88 | 313 |
| Amino Acid Metabolism | 74 | 10 | 84 |
| Carbohydrate Metabolism | 80 | 28 | 108 |
| Cellular Process and Signaling | 320 | 80 | 400 |
| Signal Transduction | 42 | 46 | 88 |
| Poorly Characterized | 302 | 102 | 404 |
| No Annotation | 840 | 524 | 1364 |
| Total | 2497 | 934 | 3431 |

Genes or DNA fragments from the leaf tissues that show at least two fold difference in expression levels between those soybean plants grown under normal water condition and those under drought conditions are listed in Table 2. Sequences for the genes listed in Tables 2 and 4 can be found in GenBank, a nucleotide and protein sequence database maintained by the National Center for Biotechnology Information (NCBI). In Table 2, the term FC stands for fold change, which represents the difference in transcript levels of an individual gene in a plant under normal condition as compared to a plant under water deficit condition as described herein. “Direction” indicates whether the change is an increase or a decrease. “PublicID” refers to the GenBank accession # or other IDs used in other publically available databases maintained by NCBI. The column under “Description and possible Function” contains functional description of a DRG or its homolog.

TABLE 2
List of Soybean Leaf genes or fragments with at least 2 fold differential expression
under normal or drought conditions

| | | | | | | | | | % | | |
|---|---|---|---|---|---|---|---|---|---|---|---|
| Item No. | FC | Direction | P Value | adjPVal | Public ID | Query_Length | Iden_numero | Iden_denom | identity | E-value | Description and possible Function |
| 1 | 2.000140815 | down | 0.007062387 | 0.03730826 | BM092139 | 411 | 36 | 50 | 72 | 6e−14 | |
| 2 | 2.000486653 | down | 0.003246677 | 0.022118419 | BM522506 | | | | | | |
| 3 | 2.00117375 | down | 0.002934264 | 0.020685994 | BI945588 | 469 | 15 | 27 | 55 | 0.12 | |
| 4 | 2.001423255 | down | 0.003446792 | 0.022938362 | CA800336 | 520 | 55 | 167 | 32 | 2e−15 | sodium ion transport |
| 5 | 2.002291748 | down | 0.003816368 | 0.024567662 | BI699518 | 358 | 11 | 28 | 39 | 1.5 | |
| 6 | 2.004820418 | down | 0.002508701 | 0.018685598 | CD403759 | 420 | 16 | 46 | 34 | 0.35 | |
| 7 | 2.004889846 | down | 0.002901433 | 0.020514002 | BU548272 | 712 | 119 | 166 | 71 | 1e−65 | protein amino acid phosphorylation |
| 8 | 2.005142372 | down | 0.007530361 | 0.03887018 | AW309809 | 656 | 58 | 68 | 85 | 2e−28 | beta-1\,3 glucan biosynthesis |
| 9 | 2.005813326 | down | 0.000340304 | 0.005277719 | BQ627607 | | | | | | |
| 10 | 2.007206808 | down | 0.000407887 | 0.005867922 | BI321695 | 425 | 12 | 25 | 48 | 0.62 | protein amino acid phosphorylation |
| 11 | 2.007453111 | down | 0.003043762 | 0.021174047 | BM525031 | 555 | 78 | 183 | 42 | 7e−35 | regulation of transcription\, DNA-dependent |
| 12 | 2.008511618 | down | 0.000365405 | 0.005480246 | CD417424 | 696 | 128 | 167 | 76 | 1e−70 | photosynthesis |
| 13 | 2.009067999 | down | 0.000436523 | 0.006121403 | CD403633 | 595 | 56 | 96 | 58 | 9e−25 | |
| 14 | 2.00949117 | down | 0.006417604 | 0.035045263 | AI856462 | 492 | 16 | 37 | 43 | 4.3 | cellulose biosynthesis |
| 15 | 2.009608029 | down | 0.002995476 | 0.02097391 | BE556493 | 501 | 35 | 60 | 58 | 3e−16 | vitamin K2 biosynthesis |
| 16 | 2.009777746 | down | 0.002890807 | 0.020499323 | BI972916 | 471 | 40 | 86 | 46 | 2e−12 | |
| 17 | 2.01071962 | down | 0.006330126 | 0.034769475 | BE022778 | 129 | 7 | 18 | 38 | 7.2 | electron transport |
| 18 | 2.011458763 | down | 0.000629733 | 0.007784017 | BF424030 | 439 | 48 | 144 | 33 | 2e−11 | |
| 19 | 2.015399343 | down | 0.000564601 | 0.007206273 | AW164562 | | | | | | |
| 20 | 2.016653352 | down | 0.002180445 | 0.017117299 | BI425390 | 590 | 48 | 154 | 31 | 4e−08 | response to light |
| 21 | 2.020489504 | down | 0.003138911 | 0.021627953 | CF807208 | 581 | 71 | 144 | 49 | 3e−27 | ethylene mediated signaling pathway |
| 22 | 2.020722056 | down | 0.004825915 | 0.028868 | CA939014 | 498 | 21 | 23 | 91 | 1e−05 | |
| 23 | 2.02303358 | down | 0.000587034 | 0.007417099 | BU551310 | 674 | 86 | 108 | 79 | 7e−46 | |
| 24 | 2.023769658 | down | 0.004813908 | 0.028826848 | BU763686 | 559 | 62 | 135 | 45 | 1e−27 | |
| 25 | 2.025610615 | down | 0.002168316 | 0.017075666 | BI968543 | 775 | 153 | 187 | 81 | 4e−81 | methionine metabolism |
| 26 | 2.025983604 | down | 0.001859632 | 0.015319356 | CF806242 | 652 | 95 | 193 | 49 | 6e−42 | apoptosis |
| 27 | 2.026045251 | down | 0.004996878 | 0.029556941 | AW349971 | 684 | 62 | 104 | 59 | 7e−30 | electron transport |
| 28 | 2.026073163 | down | 0.009055148 | 0.04400521 | BF598665 | 467 | 37 | 71 | 52 | 1e−13 | metal ion transport |
| 29 | 2.027197359 | down | 0.002413571 | 0.018202427 | BQ610925 | 354 | 14 | 29 | 48 | 0.13 | basic amino acid transport |
| 30 | 2.027362083 | down | 0.001332155 | 0.012398272 | BG043633 | 522 | 55 | 59 | 93 | 1e−25 | protein targeting |
| 31 | 2.03015335 | down | 0.003384865 | 0.022690845 | CD400915 | 429 | 13 | 27 | 48 | 1.4 | protein folding |
| 32 | 2.031100853 | down | 0.000237253 | 0.00427192 | BQ453915 | 339 | 34 | 36 | 94 | 1e−13 | metabolism |
| 33 | 2.031198073 | down | 0.002879687 | 0.020475721 | BQ273536 | 578 | 13 | 31 | 41 | 2.0 | |
| 34 | 2.031208914 | down | 0.001087344 | 0.010938585 | BG508249 | 393 | 33 | 91 | 36 | 1e−07 | |
| 35 | 2.032078842 | down | 0.003615976 | 0.023699482 | CD392826 | 641 | 86 | 115 | 74 | 1e−42 | Fructose-bisphosphate aldolase (FBA) |
| 36 | 2.033186362 | down | 0.006593823 | 0.035670943 | CF809244 | 533 | 19 | 56 | 33 | 0.054 | |
| 37 | 2.036088468 | down | 0.002435627 | 0.018326396 | BU551370 | | | | | | |
| 38 | 2.03918358 | down | 0.001778402 | 0.014938099 | CA819977 | 359 | 9 | 19 | 47 | 3.4 | |
| 39 | 2.041311634 | down | 0.003839026 | 0.024679734 | BU083613 | 278 | 57 | 71 | 80 | 6e−33 | |
| 40 | 2.041901449 | down | 0.000506993 | 0.006740857 | CD405423 | 579 | 66 | 137 | 48 | 6e−24 | |
| 41 | 2.042032034 | down | 0.007267108 | 0.037991493 | CD416575 | 357 | 21 | 70 | 30 | 2.5 | exocytosis |
| 42 | 2.043406882 | down | 0.000322629 | 0.005109176 | AW133420 | 326 | 13 | 27 | 48 | 5.4 | |
| 43 | 2.044487423 | down | 0.0000582 | 0.00202784 | AI443309 | 517 | 16 | 50 | 32 | 0.94 | regulation of transcription |
| 44 | 2.045951532 | down | 0.000577928 | 0.007333221 | CA938657 | 519 | 63 | 89 | 70 | 1e−31 | |
| 45 | 2.045952014 | down | 0.007613051 | 0.039157293 | CF805977 | 620 | 59 | 207 | 28 | 6e−06 | |
| 46 | 2.04652924 | down | 0.00801874 | 0.040545354 | CD391117 | 648 | 86 | 101 | 85 | 1e−48 | protein folding |
| 47 | 2.048436591 | down | 0.003069671 | 0.021303067 | BM176963 | 428 | 17 | 57 | 29 | 1.4 | oligopeptide transport |
| 48 | 2.048830767 | down | 0.004485223 | 0.027508099 | BG155364 | | | | | | |
| 49 | 2.050321891 | down | 0.007979446 | 0.040449904 | CF806291 | 466 | 24 | 53 | 45 | 9e−05 | |
| 50 | 2.052464321 | down | 0.003255209 | 0.022145311 | AW757224 | 458 | 63 | 152 | 41 | 6e−21 | |
| 51 | 2.054305966 | down | 0.001315695 | 0.012304462 | CD399104 | 567 | 31 | 46 | 67 | 3e−09 | protein amino acid dephosphorylation |
| 52 | 2.055131644 | down | 0.003031249 | 0.021129987 | BU548461 | 451 | 75 | 132 | 56 | 1e−39 | protein amino acid phosphorylation |
| 53 | 2.056192574 | down | 0.001660869 | 0.014246736 | BM568006 | 547 | 10 | 19 | 52 | 3.0 | Mo-molybdopterin cofactor biosynthesis |
| 54 | 2.059094233 | down | 0.001971482 | 0.01595642 | CK606714 | 185 | 8 | 17 | 47 | 7.1 | Cytosolic glutamine synthetase GSbeta 1 |
| 55 | 2.059493958 | down | 0.000259471 | 0.004490311 | BI322056 | 479 | 15 | 37 | 40 | 0.62 | |
| 56 | 2.060262813 | down | 0.004109411 | 0.025781558 | BE823130 | 460 | 79 | 99 | 79 | 8e−40 | |
| 57 | 2.063771012 | down | 0.001866382 | 0.015351432 | BM177533 | 421 | 50 | 103 | 48 | 3e−21 | protein amino acid phosphorylation |
| 58 | 2.066887374 | down | 0.003967009 | 0.025204022 | CA784143 | 564 | 14 | 26 | 53 | 5.5 | |
| 59 | 2.066959527 | down | 0.003296366 | 0.022348505 | CD399280 | 494 | 71 | 109 | 65 | 8e−37 | |
| 60 | 2.067747366 | down | 0.007674384 | 0.039354368 | BI971060 | 780 | 66 | 118 | 55 | 5e−36 | |
| 61 | 2.068221451 | down | 0.000433479 | 0.006090733 | AW706133 | 299 | 24 | 33 | 72 | 6e−10 | |
| 62 | 2.068953539 | down | 0.001187601 | 0.011557681 | BE823928 | 411 | 18 | 36 | 50 | 0.008 | |
| 63 | 2.069503072 | down | 0.010241409 | 0.047835635 | AF175394.1 | | | | | | resistance protein LM12 |
| 64 | 2.07020045 | down | 0.001115578 | 0.011127378 | BU084402 | 584 | 38 | 78 | 48 | 3e−12 | gluconeogenesis |
| 65 | 2.071750053 | down | 0.003929682 | 0.025029587 | CK606399 | 835 | 126 | 226 | 55 | 3e−70 | Lipoxygenase L-5 (vlxB) |
| 66 | 2.073108531 | down | 0.001525988 | 0.013483325 | AW760530 | 502 | 42 | 47 | 89 | 2e−17 | |
| 67 | 2.074104276 | down | 0.000135967 | 0.003091825 | AW132264 | 456 | 48 | 73 | 65 | 3e−26 | cell death |
| 68 | 2.074503877 | down | 0.001535236 | 0.013530077 | BQ628343 | 244 | 14 | 43 | 32 | 4.1 | |
| 69 | 2.076228055 | down | 0.002049467 | 0.016384686 | BQ628088 | 591 | 66 | 88 | 75 | 1e−30 | cation transport |
| 70 | 2.076815852 | down | 0.000164414 | 0.003441361 | CD396298 | 694 | 162 | 172 | 94 | 2e−91 | chlorophyll a/b-binding protein precursor, |
| | | | | | | | | | | | photosynthesis light harvesting in photosystem II |
| 71 | 2.077485789 | down | 0.005185963 | 0.030324827 | BQ612445 | 613 | 164 | 206 | 79 | 2e−93 | secondary cell wall biosynthesis (sensu |
| | | | | | | | | | | | Magnoliophyta) |
| 72 | 2.07804172 | down | 0.001167161 | 0.011441649 | BM526895 | 548 | 77 | 135 | 57 | 1e−34 | regulation of transcription\, DNA-dependent |
| 73 | 2.078930902 | down | 0.005097216 | 0.029957295 | BM522553 | 392 | 13 | 43 | 30 | 0.89 | mRNA processing |
| 74 | 2.084574507 | down | 0.003615018 | 0.023699302 | CA800304 | 750 | 112 | 249 | 44 | 2e−58 | response to wounding |
| 75 | 2.087080248 | down | 0.009660186 | 0.045948184 | BE820999 | 487 | 68 | 99 | 68 | 2e−37 | regulation of transcription\, DNA- |
| | | | | | | | | | | | dependent |
| 76 | 2.08819518 | down | 0.003493016 | 0.023131307 | BI498476 | 413 | 43 | 123 | 34 | 4e−18 | electron transport |
| 77 | 2.088406985 | down | 0.008665846 | 0.042748474 | CD400746 | 424 | 50 | 79 | 63 | 1e−21 | |
| 78 | 2.090959394 | down | 0.00465927 | 0.028180382 | BQ298018 | 609 | 103 | 203 | 50 | 9e−55 | electron transport |
| 79 | 2.091445137 | down | 0.001293114 | 0.01219658 | BE821322 | 639 | 47 | 86 | 54 | 2e−19 | |
| 80 | 2.094834916 | down | 0.00261183 | 0.019199014 | AW707230 | 506 | 14 | 16 | 87 | 0.31 | |
| 81 | 2.094987023 | down | 0.00036613 | 0.005485846 | BI784952 | 534 | 8 | 16 | 50 | 6.6 | electron transport |
| 82 | 2.095301151 | down | 0.000250426 | 0.004407052 | BI974909 | 552 | 43 | 98 | 43 | 1e−11 | |
| 83 | 2.095726992 | down | 0.007618895 | 0.039181995 | BE821413 | 663 | 91 | 166 | 54 | 1e−39 | |
| 84 | 2.096760215 | down | 0.005722845 | 0.032374654 | CD406079 | 652 | 117 | 146 | 80 | 2e−68 | brassinosteroid mediated signaling |
| 85 | 2.103394271 | down | 0.000430108 | 0.006068296 | CD412146 | 648 | 62 | 133 | 46 | 3e−27 | |
| 86 | 2.106075389 | down | 0.009581495 | 0.045753252 | CA850791 | 557 | 19 | 78 | 24 | 0.099 | response to cold |
| 87 | 2.106423186 | down | 0.001892125 | 0.015502152 | BI317008 | 434 | 20 | 78 | 25 | 3.3 | RNA dependent DNA replication |
| 88 | 2.107237797 | down | 0.001200625 | 0.011628744 | BQ628871 | 463 | 25 | 84 | 29 | 2.9 | protein amino acid phosphorylation |
| 89 | 2.108300448 | down | 0.008551446 | 0.042428977 | BU544018 | 651 | 40 | 87 | 45 | 3e−11 | |
| 90 | 2.108924894 | down | 0.000083 | 0.002432537 | BI471946 | 499 | 57 | 128 | 44 | 4e−25 | |
| 91 | 2.109644963 | down | 0.000262849 | 0.00452485 | BU082460 | 588 | 29 | 50 | 58 | 4e−11 | aromatic compound metabolism |
| 92 | 2.109823473 | down | 0.002329665 | 0.017756436 | BM143105 | 448 | 40 | 55 | 72 | 1e−16 | |
| 93 | 2.113683031 | down | 0.000202608 | 0.003880291 | BM568079 | 388 | 21 | 51 | 41 | 7e−06 | protein amino acid phosphorylation |
| 94 | 2.114473907 | down | 0.003899931 | 0.024935249 | BG508616 | 518 | 10 | 25 | 40 | 8.1 | electron transport |
| 95 | 2.11452091 | down | 0.010095151 | 0.047334707 | BQ611599 | 418 | 15 | 57 | 26 | 1.0 | cation transport |
| 96 | 2.115177474 | down | 0.002689519 | 0.019574993 | BQ611063 | 356 | 12 | 37 | 32 | 0.86 | |
| 97 | 2.117311534 | down | 0.005445433 | 0.031318734 | CD396936 | 666 | 108 | 168 | 64 | 4e−65 | |
| 98 | 2.117314098 | down | 0.004732701 | 0.028487091 | CD399954 | 465 | 26 | 49 | 53 | 2e−10 | biological_process unknown |
| 99 | 2.11876276 | down | 0.004252394 | 0.02642852 | CF805753 | 515 | 64 | 151 | 42 | 1e−19 | response to wounding |
| 100 | 2.119972008 | down | 0.000922805 | 0.009842516 | BM890692 | 575 | 137 | 187 | 73 | 3e−79 | neurotransmitter transport |
| 101 | 2.120128125 | down | 0.000499763 | 0.006670715 | CK606172 | 800 | 168 | 216 | 77 | e−100 | phenylpropanoid biosynthesis |
| 102 | 2.121876864 | down | 0.003615319 | 0.023699302 | CD409089 | 613 | 87 | 104 | 83 | 3e−47 | beta-1\,3 glucan biosynthesis |
| 103 | 2.123140105 | down | 0.001073153 | 0.010877274 | BI273666 | | | | | | |
| 104 | 2.126700533 | down | 0.000416955 | 0.005948834 | BI968053 | 525 | 23 | 67 | 34 | 1.7 | |
| 105 | 2.127506119 | down | 0.010627267 | 0.049017049 | CD412631 | 707 | 78 | 140 | 55 | 4e−39 | |
| 106 | 2.127843873 | down | 0.001520828 | 0.013466194 | BE331512 | 530 | 18 | 50 | 36 | 2.9 | nucleotide metabolism |
| 107 | 2.127878977 | down | 0.007169107 | 0.03764754 | BQ296209 | | | | | | |
| 108 | 2.129126678 | down | 0.007142765 | 0.037586452 | AW350412 | 629 | 50 | 86 | 58 | 2e−22 | |
| 109 | 2.130265635 | down | 0.000277522 | 0.004678935 | CD416063 | 405 | 64 | 90 | 71 | 4e−32 | L-serine biosynthesis |
| 110 | 2.130711774 | down | 0.005002729 | 0.029585228 | BU544647 | 576 | 57 | 120 | 47 | 2e−24 | protein amino acid phosphorylation |
| 111 | 2.131205658 | down | 0.000807316 | 0.009059185 | CF808307 | 431 | 58 | 101 | 57 | 1e−30 | electron transport |
| 112 | 2.131948906 | down | 0.001268606 | 0.012028774 | CD412259 | 224 | 14 | 34 | 41 | 3.2 | electron transport |
| 113 | 2.132977375 | down | 0.001328741 | 0.012377234 | BU548459 | 496 | 39 | 110 | 35 | 2e−12 | |
| 114 | 2.133227352 | down | 0.003345554 | 0.022557756 | BE821395 | | | | | | |
| 115 | 2.133272707 | down | 0.004621188 | 0.028026662 | BG510290 | 495 | 130 | 160 | 81 | 1e−71 | protein amino acid phosphorylation |
| 116 | 2.133361127 | down | 0.004548127 | 0.027731117 | BM522465 | 293 | 9 | 20 | 45 | 1.9 | |
| 117 | 2.135714298 | down | 0.000110124 | 0.002789365 | BI970506 | 742 | 28 | 100 | 28 | 0.001 | protein amino acid phosphorylation |
| 118 | 2.136863263 | down | 0.004856882 | 0.028988695 | CA784613 | 756 | 215 | 252 | 85 | e−123 | carbohydrate metabolism |
| 119 | 2.137341426 | down | 0.000751833 | 0.00869602 | BU578146 | 573 | 93 | 150 | 62 | 4e−47 | response to salicylic acid stimulus |
| 120 | 2.137682858 | down | 0.004120068 | 0.025833009 | BI321301 | | | | | | |
| 121 | 2.1390826 | down | 0.002281308 | 0.017529956 | BM139615 | 266 | 13 | 43 | 30 | 1.1 | protein amino acid phosphorylation |
| 122 | 2.139601889 | down | 0.001608392 | 0.013939648 | AI938354 | 169 | 15 | 38 | 39 | 4.2 | |
| 123 | 2.141964664 | down | 0.00247198 | 0.018514746 | CD413441 | 538 | 19 | 32 | 59 | 1e−04 | electron transport |
| 124 | 2.145964865 | down | 0.004933352 | 0.029291727 | CD417493 | 699 | 98 | 140 | 70 | 5e−53 | |
| 125 | 2.150676477 | down | 0.000786794 | 0.008937084 | AF537220.1 | | | | | | ethylene responsive protein |
| 126 | 2.152413065 | down | 0.0000564 | 0.002005588 | CD410928 | 349 | 16 | 48 | 33 | 2.5 | intracellular protein transport |
| 127 | 2.154223068 | down | 0.000405781 | 0.005858599 | BI702282 | | | | | | |
| 128 | 2.154382692 | down | 0.009813879 | 0.04645014 | CA784898 | 760 | 62 | 212 | 29 | 2e−08 | |
| 129 | 2.156999375 | down | 0.003329158 | 0.022519136 | CD406471 | 570 | 31 | 46 | 67 | 3e−16 | |
| 130 | 2.158334714 | down | 0.000367636 | 0.005490568 | BE658339 | 391 | 27 | 92 | 29 | 0.004 | |
| 131 | 2.158476709 | down | 0.002944211 | 0.020730728 | CF807507 | 457 | 36 | 46 | 78 | 3e−17 | fatty acid alpha-oxidation |
| 132 | 2.159248257 | down | 0.000375989 | 0.005575844 | BE057315 | 245 | 13 | 31 | 41 | 2.4 | proteolysis and peptidolysis |
| 133 | 2.159659391 | down | 0.000449844 | 0.006262193 | AY323131.1 | | | | | | GAD protein |
| 134 | 2.160399838 | down | 0.00571852 | 0.032364792 | BU546346 | 607 | 13 | 39 | 33 | 6.3 | |
| 135 | 2.16149233 | down | 0.001138409 | 0.011274389 | AW397591 | 311 | 10 | 21 | 47 | 4.2 | |
| 136 | 2.161493999 | down | 0.010028109 | 0.047084921 | U43839.1 | | | | | | GmCK2p |
| 137 | 2.162984414 | down | 0.002887679 | 0.020497672 | BU544472 | 651 | 32 | 124 | 25 | 4e−07 | metabolism |
| 138 | 2.166407289 | down | 0.001620039 | 0.014011534 | BQ298229 | | | | | | |
| 139 | 2.169899238 | down | 0.005799953 | 0.03268306 | BG237455 | 211 | 10 | 20 | 50 | 2.5 | |
| 140 | 2.170194083 | down | 0.009105502 | 0.044181418 | BQ612822 | 431 | 12 | 13 | 92 | 0.17 | |
| 141 | 2.173783688 | down | 0.010233468 | 0.047810419 | CA850791 | 557 | 19 | 78 | 24 | 0.099 | response to cold |
| 142 | 2.176325299 | down | 0.006130203 | 0.033958976 | BG510290 | 495 | 130 | 160 | 81 | 1e−71 | protein amino acid phosphorylation |
| 143 | 2.177064564 | down | 0.000334098 | 0.005229615 | AW310754 | 374 | 23 | 56 | 41 | 6e−08 | carbohydrate metabolism |
| 144 | 2.177556464 | down | 0.009902745 | 0.046717905 | BE820973 | 704 | 80 | 126 | 63 | 3e−39 | flower development |
| 145 | 2.177846323 | down | 0.008794648 | 0.043163086 | BU545410 | 550 | 31 | 51 | 60 | 1e−10 | phototropism |
| 146 | 2.178630044 | down | 0.001344393 | 0.012461308 | CA938457 | 333 | 25 | 45 | 55 | 2e−08 | |
| 147 | 2.179178729 | down | 0.002759511 | 0.019884341 | AB086391.1 | | | | | | simillar to Arabidopsis thaliana putative disease |
| | | | | | | | | | | | resistance protein |
| 148 | 2.181212903 | down | 0.000323992 | 0.005122583 | BE820147 | 285 | 8 | 28 | 28 | 9.2 | |
| 149 | 2.181562359 | down | 0.000633147 | 0.007811531 | BE022504 | 372 | 10 | 24 | 41 | 1.00 | protein amino acid phosphorylation |
| 150 | 2.181895812 | down | 0.002051611 | 0.016384686 | AW310139 | 676 | 53 | 77 | 68 | 5e−26 | protein amino acid phosphorylation |
| 151 | 2.182595288 | down | 0.000220255 | 0.004081121 | AW397682 | 471 | 59 | 95 | 62 | 2e−26 | |
| 152 | 2.183769029 | down | 0.003335076 | 0.022525689 | CD414940 | 471 | 12 | 35 | 34 | 0.46 | protein amino acid phosphorylation |
| 153 | 2.183869903 | down | 0.002051509 | 0.016384686 | CF808455 | 320 | 17 | 22 | 77 | 3e−04 | amino acid transport |
| 154 | 2.183926286 | down | 0.0000299 | 0.00147742 | BQ630585 | | | | | | |
| 155 | 2.18564035 | down | 0.0000257 | 0.001377579 | BQ297707 | 624 | 95 | 200 | 47 | 1e−42 | G1 phase of mitotic cell cycle |
| 156 | 2.186292248 | down | 0.010704922 | 0.049284568 | CD409745 | 438 | 36 | 82 | 43 | 6e−13 | flower development |
| 157 | 2.188231724 | down | 0.002374111 | 0.018003991 | BM085386 | 513 | 97 | 153 | 63 | 1e−49 | |
| 158 | 2.190175273 | down | 0.007749916 | 0.039633626 | BE658944 | 398 | 9 | 20 | 45 | 5.9 | |
| 159 | 2.191483924 | down | 0.010532166 | 0.048661946 | BF595464 | 443 | 32 | 66 | 48 | 1e−08 | transport |
| 160 | 2.193179361 | down | 0.005480545 | 0.031445766 | BG509003 | 369 | 16 | 56 | 28 | 0.57 | |
| 161 | 2.194106915 | down | 0.000155096 | 0.003324117 | CF809320 | 516 | 20 | 36 | 55 | 0.002 | |
| 162 | 2.194946357 | down | 0.007939507 | 0.040302181 | CF807824 | 568 | 104 | 171 | 60 | 7e−57 | |
| 163 | 2.195116541 | down | 0.000101888 | 0.002670663 | BG238723 | 468 | 48 | 80 | 60 | 2e−22 | electron transport |
| 164 | 2.195466157 | down | 0.0000597 | 0.002061297 | BM271089 | 479 | 131 | 162 | 80 | 7e−71 | protein amino acid phosphorylation |
| 165 | 2.195997825 | down | 0.001490645 | 0.01328652 | BI969613 | 699 | 35 | 48 | 72 | 1e−13 | |
| 166 | 2.196358168 | down | 0.004886318 | 0.029127412 | BM524135 | 531 | 15 | 44 | 34 | 2.9 | |
| 167 | 2.197358946 | down | 0.001542991 | 0.013579334 | AW424283 | 445 | 22 | 30 | 73 | 8e−07 | |
| 168 | 2.201564842 | down | 0.008047501 | 0.040657985 | BU545972 | 537 | 77 | 83 | 92 | 7e−40 | carbohydrate metabolism |
| 169 | 2.201829925 | down | 0.000568234 | 0.00724527 | BG156885 | 247 | 14 | 50 | 28 | 1.9 | |
| 170 | 2.201938634 | down | 0.000237563 | 0.004275452 | CA853083 | 222 | 46 | 55 | 83 | 4e−22 | protein amino acid phosphorylation |
| 171 | 2.202061778 | down | 0.007152441 | 0.03761106 | CA937103 | 335 | 17 | 28 | 60 | 6e−04 | |
| 172 | 2.205735813 | down | 0.003199486 | 0.021877018 | BE804860 | 626 | 78 | 94 | 82 | 2e−38 | |
| 173 | 2.207273997 | down | 0.000201772 | 0.003868666 | CA819289 | 582 | 66 | 117 | 56 | 2e−37 | |
| 174 | 2.20774385 | down | 0.003759769 | 0.024303137 | AW348549 | 715 | 122 | 204 | 59 | 4e−68 | metabolism |
| 175 | 2.211222376 | down | 0.000143873 | 0.003182607 | BM522670 | 383 | 11 | 15 | 73 | 0.48 | |
| 176 | 2.212043797 | down | 0.005549 | 0.031701107 | BG509584 | 339 | 27 | 36 | 75 | 1e−11 | |
| 177 | 2.213199598 | down | 0.002241071 | 0.017342117 | BQ627887 | 566 | 23 | 55 | 41 | 8e−05 | |
| 178 | 2.213634565 | down | 0.000240334 | 0.004302823 | CA819417 | 216 | 11 | 21 | 52 | 9.4 | |
| 179 | 2.214827347 | down | 0.004538545 | 0.027709387 | BG726161 | 399 | 14 | 23 | 60 | 0.53 | |
| 180 | 2.215818294 | down | 0.000497493 | 0.006654586 | BQ743019 | 368 | 25 | 25 | 100 | 2e−09 | positive regulation of flower |
| | | | | | | | | | | | development |
| 181 | 2.219722354 | down | 0.0000143 | 0.001114494 | BM188674 | 326 | 10 | 13 | 76 | 1.8 | regulation of transcription\, DNA- |
| | | | | | | | | | | | dependent |
| 182 | 2.223298611 | down | 0.0000427 | 0.001761872 | BI427232 | 555 | 103 | 181 | 56 | 2e−54 | protein amino acid phosphorylation |
| 183 | 2.223690551 | down | 0.001658433 | 0.014235576 | CD395825 | 653 | 133 | 142 | 93 | 3e−73 | Myo-inositol-3-phosphate synthase (MIPS), myo- |
| | | | | | | | | | | | inositol biosynthesis |
| 184 | 2.225180145 | down | 0.000352541 | 0.005371525 | BE329841 | 461 | 13 | 46 | 28 | 6.4 | |
| 185 | 2.227677166 | down | 0.004809578 | 0.028815489 | CF806372 | 533 | 50 | 102 | 49 | 3e−16 | |
| 186 | 2.228577659 | down | 0.003796676 | 0.024470214 | BG551162 | 498 | 140 | 165 | 84 | 9e−81 | |
| 187 | 2.230234507 | down | 0.001046691 | 0.01071299 | CA937905 | 534 | 143 | 179 | 79 | 2e−77 | |
| 188 | 2.230728585 | down | 0.001306442 | 0.012266724 | BM731975 | 534 | 106 | 174 | 60 | 5e−64 | |
| 189 | 2.233128074 | down | 0.0000837 | 0.002444271 | BE607604 | 436 | 44 | 53 | 83 | 3e−20 | |
| 190 | 2.23377688 | down | 0.006869305 | 0.036603208 | AW101694 | 307 | 17 | 60 | 28 | 0.50 | |
| 191 | 2.233882571 | down | 0.003279395 | 0.022277614 | BU545628 | 542 | 25 | 87 | 28 | 0.36 | |
| 192 | 2.234107196 | down | 0.002495582 | 0.018624769 | BM143123 | 316 | 34 | 66 | 51 | 2e−10 | |
| 193 | 2.235124296 | down | 0.003379016 | 0.022671835 | AW569785 | 258 | 20 | 41 | 48 | 3e−04 | |
| 194 | 2.237048005 | down | 0.002173702 | 0.017098026 | CD406171 | 552 | 62 | 113 | 54 | 4e−35 | |
| 195 | 2.24213843 | down | 0.001532711 | 0.013514161 | CD408395 | 662 | 59 | 133 | 44 | 2e−19 | |
| 196 | 2.243373686 | down | 0.000190106 | 0.003734024 | BE611653 | 367 | 24 | 81 | 29 | 0.065 | |
| 197 | 2.244331651 | down | 0.002681426 | 0.019552974 | CD396390 | 672 | 124 | 162 | 76 | 5e−71 | |
| 198 | 2.244578234 | down | 0.007300671 | 0.038087024 | BQ630327 | 571 | 40 | 74 | 54 | 8e−15 | proteolysis and peptidolysis |
| 199 | 2.245347827 | down | 0.006134133 | 0.03396833 | CD412626 | 415 | 16 | 32 | 50 | 0.35 | |
| 200 | 2.245803437 | down | 0.00731213 | 0.038130936 | BI971119 | 782 | 97 | 112 | 86 | 4e−54 | purine ribonucleoside monophosphate biosynthesis |
| 201 | 2.247931567 | down | 0.010730202 | 0.049334535 | CA935591 | 423 | 13 | 38 | 34 | 8.9 | metabolism |
| 202 | 2.248764012 | down | 0.007752271 | 0.039634895 | CD393581 | 604 | 72 | 134 | 53 | 2e−32 | |
| 203 | 2.249054069 | down | 0.001381725 | 0.012661014 | BF598754 | 439 | 21 | 22 | 95 | 1e−06 | electron transport |
| 204 | 2.251281312 | down | 0.00074315 | 0.008640755 | AW397797 | 421 | 22 | 59 | 37 | 0.78 | regulation of carbohydrate metabolism |
| 205 | 2.251433814 | down | 0.0000798 | 0.002384832 | AW310615 | 616 | 56 | 127 | 44 | 4e−20 | protein amino acid phosphorylation |
| 206 | 2.25409234 | down | 0.00127349 | 0.012056644 | BG510307 | 294 | 13 | 40 | 32 | 7.2 | |
| 207 | 2.255025116 | down | 0.004413333 | 0.027181377 | CF807323 | 751 | 73 | 163 | 44 | 2e−39 | response to pathogenic fungi |
| 208 | 2.255765196 | down | 0.000143445 | 0.003181853 | BQ298627 | 460 | 15 | 24 | 62 | 0.030 | protein amino acid phosphorylation |
| 209 | 2.256700329 | down | 0.001486392 | 0.013259916 | BG405383 | 420 | 10 | 29 | 34 | 5.1 | |
| 210 | 2.25848202 | down | 0.006558556 | 0.035531258 | BI967327 | 770 | 149 | 163 | 91 | 4e−87 | |
| 211 | 2.258731467 | down | 0.001994506 | 0.016105914 | BE346680 | 643 | 144 | 214 | 67 | 3e−83 | signal transduction |
| 212 | 2.260687431 | down | 0.000440555 | 0.006169934 | AW394946 | 411 | 19 | 20 | 95 | 7e−05 | |
| 213 | 2.260753144 | down | 0.003364744 | 0.022626124 | BE659422 | 423 | 13 | 28 | 46 | 0.16 | |
| 214 | 2.261904314 | down | 0.000630513 | 0.007791105 | CD399550 | 713 | 87 | 128 | 67 | 1e−47 | |
| 215 | 2.261944408 | down | 0.002564663 | 0.018948583 | CD399841 | 483 | 20 | 36 | 55 | 0.002 | |
| 216 | 2.262938839 | down | 0.000455495 | 0.006314252 | BG510614 | 503 | 54 | 70 | 77 | 5e−28 | |
| 217 | 2.262967934 | down | 0.005395434 | 0.031107275 | BG508205 | 312 | 36 | 41 | 87 | 3e−17 | alternative oxidase 2b |
| 218 | 2.263153611 | down | 0.006575274 | 0.035585951 | BM085346 | 540 | 32 | 36 | 88 | 4e−13 | cation transport |
| 219 | 2.263722455 | down | 0.010261658 | 0.047876704 | AW102068 | 470 | 53 | 82 | 64 | 6e−27 | lipid catabolism |
| 220 | 2.266294395 | down | 0.001664381 | 0.014260597 | BM271210 | 494 | 25 | 61 | 40 | 5e−04 | |
| 221 | 2.267977737 | down | 0.0000351 | 0.001586526 | CF808907 | 620 | 135 | 209 | 64 | 3e−78 | |
| 222 | 2.268241051 | down | 0.002068306 | 0.016482467 | BG406413 | 351 | 11 | 42 | 26 | 5.4 | regulation of transcription\, DNA- |
| | | | | | | | | | | | dependent |
| 223 | 2.273615255 | down | 0.004182604 | 0.026135051 | CD405755 | 420 | 56 | 65 | 86 | 1e−29 | fatty acid metabolism |
| 224 | 2.273651874 | down | 0.002953154 | 0.020770368 | BG508510 | 334 | 14 | 38 | 36 | 0.85 | |
| 225 | 2.274531638 | down | 0.00029991 | 0.004877132 | AW394946 | 411 | 19 | 20 | 95 | 7e−05 | |
| 226 | 2.274587834 | down | 0.005721325 | 0.032374654 | CA937161 | 444 | 51 | 79 | 64 | 2e−25 | red\, far-red light phototransduction |
| 227 | 2.275617394 | down | 0.000638928 | 0.007844377 | BU083528 | 579 | 54 | 105 | 51 | 2e−21 | transport |
| 228 | 2.278752618 | down | 0.0001671 | 0.003478297 | BQ454274 | 439 | 13 | 36 | 36 | 0.67 | |
| 229 | 2.27886503 | down | 0.001992734 | 0.016095058 | BU546941 | 643 | 39 | 99 | 39 | 1e−12 | aging |
| 230 | 2.278991489 | down | 0.000131232 | 0.003039857 | BI320672 | | | | | | |
| 231 | 2.279736365 | down | 0.00057862 | 0.007337898 | AW781094 | 406 | 12 | 34 | 35 | 8.0 | nucleotide-excision repair |
| 232 | 2.280534416 | down | 0.0000308 | 0.001495932 | CD405129 | 558 | 41 | 101 | 40 | 8e−18 | |
| 233 | 2.283165913 | down | 0.002599806 | 0.019124234 | BG726570 | 409 | 20 | 58 | 34 | 0.97 | RNA processing |
| 234 | 2.284348024 | down | 0.00324264 | 0.022099776 | CD402638 | 362 | 20 | 34 | 58 | 4e−05 | |
| 235 | 2.285037423 | down | 0.00100641 | 0.010445666 | CD399334 | 628 | 25 | 34 | 73 | 5e−08 | |
| 236 | 2.286565335 | down | 0.001665731 | 0.014268916 | AJ563365.1 | | | | | | monosaccharide transporter |
| 237 | 2.287483668 | down | 0.000262581 | 0.004522303 | BM178538 | 393 | 16 | 46 | 34 | 0.14 | regulation of translational initiation |
| 238 | 2.290447128 | down | 0.0000822 | 0.002420756 | BE657993 | 436 | 13 | 24 | 54 | 0.060 | protein amino acid phosphorylation |
| 239 | 2.290743528 | down | 0.005236332 | 0.030507173 | BG508937 | 373 | 15 | 31 | 48 | 2.2 | |
| 240 | 2.291033412 | down | 0.000654041 | 0.007948662 | BM188434 | 569 | 117 | 181 | 64 | 5e−68 | protein amino acid phosphorylation |
| 241 | 2.291559882 | down | 0.003000244 | 0.020999472 | BM525005 | 428 | 16 | 36 | 44 | 1.4 | |
| 242 | 2.293446117 | down | 0.009559127 | 0.045675433 | AW307396 | 403 | 19 | 53 | 35 | 0.24 | G-protein coupled receptor protein |
| | | | | | | | | | | | signaling pathway |
| 243 | 2.301581964 | down | 0.009710689 | 0.046118415 | BG157397 | 383 | 9 | 24 | 37 | 5.3 | |
| 244 | 2.302311933 | down | 0.004619662 | 0.028026446 | BI785018 | | | | | | |
| 245 | 2.303456681 | down | 0.000373645 | 0.005554873 | CD403268 | 487 | 37 | 88 | 42 | 7e−11 | response to wounding |
| 246 | 2.304536823 | down | 0.000639552 | 0.007844432 | BU546369 | 607 | 96 | 137 | 70 | 2e−47 | |
| 247 | 2.306029958 | down | 0.007744718 | 0.039624126 | BI424658 | 591 | 116 | 197 | 58 | 3e−64 | trehalose biosynthesis |
| 248 | 2.306498643 | down | 0.003897388 | 0.024929379 | CK605667 | 746 | 144 | 167 | 86 | 1e−77 | methionine metabolism |
| 249 | 2.308103661 | down | 0.000534112 | 0.006951941 | BE209713 | 280 | 14 | 16 | 87 | 0.034 | |
| 250 | 2.309598622 | down | 0.000596264 | 0.00750347 | CD418234 | 402 | 14 | 46 | 30 | 6.0 | |
| 251 | 2.311228859 | down | 0.000164573 | 0.003441361 | BU550106 | 563 | 78 | 118 | 66 | 6e−42 | |
| 252 | 2.312225505 | down | 0.005091108 | 0.029939148 | BI892928 | 214 | 10 | 31 | 32 | 1.9 | protein amino acid phosphorylation |
| 253 | 2.315409015 | down | 0.006591218 | 0.035662747 | CD406446 | 490 | 17 | 48 | 35 | 2.5 | |
| 254 | 2.315422946 | down | 0.000882212 | 0.009575935 | BE612219 | 859 | 134 | 179 | 74 | 4e−74 | photosynthesis |
| 255 | 2.315979928 | down | 0.010884082 | 0.049834921 | BQ452666 | 438 | 33 | 39 | 84 | 6e−15 | |
| 256 | 2.317016498 | down | 0.000604539 | 0.00757604 | AW133007 | 515 | 103 | 156 | 66 | 4e−48 | regulation of transcription\, DNA- |
| | | | | | | | | | | | dependent |
| 257 | 2.317533205 | down | 0.006221846 | 0.034324384 | BU546655 | 639 | 159 | 167 | 95 | 4e−92 | microtubule-based process |
| 258 | 2.318656987 | down | 0.0000111 | 0.000993386 | BU548662 | 690 | 75 | 143 | 52 | 1e−33 | ethylene mediated signaling pathway |
| 259 | 2.319467728 | down | 0.000456432 | 0.006321149 | CD394372 | 470 | 21 | 41 | 51 | 2e−05 | protein amino acid phosphorylation |
| 260 | 2.319917724 | down | 0.009637129 | 0.045908176 | BE823413 | 671 | 21 | 44 | 47 | 0.007 | |
| 261 | 2.321066726 | down | 0.001809363 | 0.015090326 | BU547306 | 547 | 14 | 20 | 70 | 0.12 | |
| 262 | 2.322419314 | down | 0.000657486 | 0.007972497 | BE058386 | 434 | 27 | 68 | 39 | 4e−04 | |
| 263 | 2.322964253 | down | 0.006483453 | 0.035256411 | BG790659 | 468 | 58 | 93 | 62 | 1e−27 | regulation of transcription |
| 264 | 2.325152612 | down | 0.000336532 | 0.00525023 | BE824025 | 661 | 52 | 85 | 61 | 2e−24 | |
| 265 | 2.325237701 | down | 0.001151181 | 0.011353043 | BI974524 | 402 | 14 | 41 | 34 | 6.0 | |
| 266 | 2.325871728 | down | 0.000226959 | 0.00415694 | BI945149 | 327 | 16 | 55 | 29 | 2.4 | proteolysis and peptidolysis |
| 267 | 2.327134914 | down | 0.000141136 | 0.003154302 | BI788296 | 558 | 27 | 68 | 39 | 6e−04 | |
| 268 | 2.329422451 | down | 0.001246515 | 0.011882012 | BM271110 | 429 | 96 | 121 | 79 | 1e−49 | transport |
| 269 | 2.329460681 | down | 0.002255379 | 0.017419679 | CF808749 | 565 | 15 | 44 | 34 | 4.2 | |
| 270 | 2.332308962 | down | 0.005035767 | 0.029703217 | BU578968 | 430 | 11 | 30 | 36 | 5.4 | electron transport |
| 271 | 2.332459991 | down | 0.005661232 | 0.032142206 | CD415914 | 589 | 58 | 62 | 93 | 1e−29 | |
| 272 | 2.332831923 | down | 0.000903101 | 0.009706107 | BG650176 | 384 | 14 | 48 | 29 | 1.8 | |
| 273 | 2.333003985 | down | 0.00041357 | 0.005910346 | BU763592 | 419 | 15 | 67 | 22 | 6.6 | regulation of transcription |
| 274 | 2.333948774 | down | 0.001697238 | 0.014462803 | CK606552 | 636 | 160 | 184 | 86 | 2e−91 | |
| 275 | 2.335000322 | down | 0.006069633 | 0.033702861 | CF809350 | 751 | 130 | 243 | 53 | 2e−68 | 4-coumarate:coenzyme A ligase, |
| | | | | | | | | | | | phenylpropanoid metabolism |
| 276 | 2.336512939 | down | 0.001885395 | 0.015463855 | CD409604 | 687 | 106 | 136 | 77 | 6e−59 | |
| 277 | 2.336623705 | down | 0.005123366 | 0.030046514 | BE659749 | 397 | 18 | 68 | 26 | 1.5 | proteolysis and peptidolysis |
| 278 | 2.337790759 | down | 0.000977762 | 0.01020593 | AI938916 | 342 | 15 | 43 | 34 | 9.4 | protein amino acid phosphorylation |
| 279 | 2.338461293 | down | 0.002362372 | 0.017936672 | AI939229 | 408 | 12 | 12 | 100 | 4.8 | |
| 280 | 2.341072118 | down | 0.003753825 | 0.024285558 | AW349256 | 729 | 25 | 92 | 27 | 0.12 | |
| 281 | 2.342044906 | down | 0.00002 | 0.001265458 | BE823876 | 710 | 102 | 151 | 67 | 6e−53 | proteolysis and peptidolysis |
| 282 | 2.343535689 | down | 0.01038556 | 0.048245333 | BE822041 | 681 | 90 | 127 | 70 | 5e−47 | |
| 283 | 2.347854638 | down | 0.006270771 | 0.034519067 | AW707191 | 555 | 80 | 155 | 51 | 2e−38 | regulation of transcription |
| 284 | 2.351109375 | down | 0.000748546 | 0.008672578 | AI442578 | 407 | 14 | 43 | 32 | 0.97 | |
| 285 | 2.351377196 | down | 0.00098064 | 0.010220504 | BI945486 | 372 | 11 | 25 | 44 | 0.77 | protein amino acid phosphorylation |
| 286 | 2.351945329 | down | 0.006195097 | 0.03420261 | BE023715 | 365 | 14 | 48 | 29 | 4.7 | transport |
| 287 | 2.352212118 | down | 0.000340244 | 0.005277719 | BG047555 | 501 | 17 | 47 | 36 | 1.5 | metal ion transport |
| 288 | 2.35595839 | down | 0.002776417 | 0.019967909 | BI970041 | 793 | 110 | 152 | 72 | 3e−57 | exocytosis |
| 289 | 2.356172375 | down | 0.000228995 | 0.004179238 | BM523017 | 399 | 27 | 73 | 36 | 1e−05 | |
| 290 | 2.356419435 | down | 0.005826241 | 0.032782084 | BE658881 | 604 | 43 | 102 | 42 | 6e−13 | salinity response |
| 291 | 2.358546723 | down | 0.003129276 | 0.021577378 | BU549358 | 372 | 15 | 39 | 38 | 2.9 | defense response |
| 292 | 2.36028609 | down | 0.003590792 | 0.023593894 | CD412200 | 620 | 30 | 35 | 85 | 3e−12 | |
| 293 | 2.361551879 | down | 0.0000134 | 0.001091338 | AW310916 | 535 | 55 | 95 | 57 | 1e−29 | basic amino acid transport |
| 294 | 2.365206336 | down | 0.00058742 | 0.007419476 | BQ452906 | 575 | 13 | 19 | 68 | 0.062 | regulation of transcription\, DNA- |
| | | | | | | | | | | | dependent |
| 295 | 2.367557793 | down | 0.001825554 | 0.015168185 | BU548420 | 375 | 14 | 33 | 42 | 1.3 | |
| 296 | 2.370362585 | down | 0.007832381 | 0.039892646 | CF808691 | 498 | 144 | 165 | 87 | 6e−82 | glutamate dehydrogenase 1, electron |
| | | | | | | | | | | | transport |
| 297 | 2.373240027 | down | 0.002636889 | 0.019337219 | AW349752 | 618 | 53 | 102 | 51 | 9e−23 | N-terminal protein amino acid |
| | | | | | | | | | | | acetylation |
| 298 | 2.37522851 | down | 0.000746624 | 0.008659739 | AW201322 | 379 | 66 | 102 | 64 | 2e−36 | |
| 299 | 2.375308907 | down | 0.002111112 | 0.016713823 | BF009782 | 308 | 16 | 35 | 45 | 0.84 | regulation of transcription\, DNA- |
| | | | | | | | | | | | dependent |
| 300 | 2.378236127 | down | 0.003508571 | 0.0232058 | BE658979 | 752 | 43 | 86 | 50 | 4e−22 | similar to NP_071584.1 dual |
| | | | | | | | | | | | specificity phosphatase 12 [Rattus |
| | | | | | | | | | | | norvegicus] |
| 301 | 2.385608376 | down | 0.002327004 | 0.017749833 | BU549296 | 628 | 35 | 62 | 56 | 3e−15 | protein amino acid phosphorylation |
| 302 | 2.3887479984 | down | 0.002041296 | 0.016350361 | AY308655.1 | | | | | | Disease resistance-like KR68 |
| | | | | | | | | | | | pseudogene |
| 303 | 2.388293798 | down | 0.002223444 | 0.017287137 | BQ742835 | 425 | 41 | 104 | 39 | 1e−11 | tRNA aminoacylation for protein |
| | | | | | | | | | | | translation |
| 304 | 2.390575936 | down | 0.001697593 | 0.014462803 | BE058510 | 349 | 17 | 47 | 36 | 0.020 | protein amino acid phosphorylation |
| 305 | 2.392092553 | down | 0.001124183 | 0.011176734 | CD390427 | 328 | 12 | 39 | 30 | 7.0 | protein amino acid glycosylation |
| 306 | 2.393031758 | down | 0.005218782 | 0.030437972 | BG155042 | 496 | 20 | 32 | 62 | 4e−05 | cation transport |
| 307 | 2.395316172 | down | 0.00000341 | 0.000750585 | BU551233 | 564 | 52 | 183 | 28 | 2e−13 | |
| 308 | 2.39711697 | down | 0.0000829 | 0.002431824 | AF502079.1 | | | | | | resistance protein KR3 |
| 309 | 2.397402854 | down | 0.002896632 | 0.020499323 | CA937161 | 444 | 51 | 79 | 64 | 2e−25 | red\, far-red light phototransduction |
| 310 | 2.400096953 | down | 0.004348961 | 0.026887215 | CD413692 | 507 | 51 | 73 | 69 | 9e−22 | |
| 311 | 2.400792079 | down | 0.004064227 | 0.025579836 | BE821142 | 410 | 16 | 41 | 39 | 0.76 | glycerol-3-phosphate metabolism |
| 312 | 2.400894955 | down | 0.002891628 | 0.020499323 | CD407578 | 683 | 66 | 81 | 81 | 2e−39 | electron transport |
| 313 | 2.40139737 | down | 0.00020064 | 0.0038555 | BE657634 | 749 | 96 | 195 | 49 | 1e−40 | |
| 314 | 2.402450131 | down | 0.006633024 | 0.035826329 | CF806101 | 552 | 102 | 186 | 54 | 2e−52 | |
| 315 | 2.404260672 | down | 0.000259629 | 0.004490311 | BE821939 | 618 | 23 | 57 | 40 | 4e−06 | regulation of transcription\, DNA- |
| | | | | | | | | | | | dependent |
| 316 | 2.404752251 | down | 0.002168331 | 0.017075666 | BQ628624 | 283 | 10 | 24 | 41 | 5.4 | |
| 317 | 2.404903872 | down | 0.006718229 | 0.036115985 | AW101682 | 417 | 34 | 60 | 56 | 5e−17 | |
| 318 | 2.406425048 | down | 0.000712512 | 0.008392264 | BQ273182 | 384 | 12 | 15 | 80 | 0.48 | |
| 319 | 2.409122835 | down | 0.009541951 | 0.045622141 | AW350114 | 760 | 122 | 199 | 61 | 7e−69 | |
| 320 | 2.412574055 | down | 0.000522902 | 0.006867477 | BE022504 | 372 | 10 | 24 | 41 | 1.00 | protein amino acid phosphorylation |
| 321 | 2.413307625 | down | 0.004060576 | 0.025566538 | CF807837 | 432 | 13 | 31 | 41 | 4.2 | carbohydrate metabolism |
| 322 | 2.418208876 | down | 0.000157375 | 0.003347279 | BE821339 | 682 | 14 | 31 | 45 | 1.2 | nucleobase\, nucleoside\, nucleotide |
| | | | | | | | | | | | and nucleic acid transport |
| 323 | 2.419837896 | down | 0.006037462 | 0.033570219 | BI469060 | 313 | 38 | 53 | 71 | 2e−13 | response to DNA damage stimulus |
| 324 | 2.421814475 | down | 0.000122405 | 0.002941938 | AW397684 | 522 | 84 | 173 | 48 | 3e−39 | |
| 325 | 2.422351559 | down | 0.002438981 | 0.018344301 | AW597705 | 378 | 13 | 34 | 38 | 0.35 | lipid metabolism |
| 326 | 2.424401551 | down | 0.0000153 | 0.001131323 | CD410650 | 647 | 57 | 127 | 44 | 5e−17 | |
| 327 | 2.425338409 | down | 0.00025132 | 0.004413227 | BE820448 | 457 | 21 | 64 | 32 | 0.33 | |
| 328 | 2.426935628 | down | 0.001238976 | 0.011843163 | CD412566 | 649 | 77 | 96 | 80 | 3e−42 | |
| 329 | 2.427412804 | down | 0.001408578 | 0.012819318 | AI442650 | 533 | 49 | 107 | 45 | 1e−27 | |
| 330 | 2.431050714 | down | 0.003519425 | 0.023249866 | AW309513 | 427 | 26 | 37 | 70 | 3e−10 | cysteine biosynthesis from serine |
| 331 | 2.431691959 | down | 0.009381859 | 0.045097666 | CD410408 | 656 | 78 | 131 | 59 | 6e−39 | |
| 332 | 2.431886251 | down | 0.003337119 | 0.022529473 | BF596552 | 313 | 8 | 16 | 50 | 1.1 | transport |
| 333 | 2.435358457 | down | 0.001307097 | 0.012266753 | AI938957 | 566 | 28 | 46 | 60 | 3e−11 | |
| 334 | 2.436005497 | down | 0.009001712 | 0.043816151 | CD403403 | 638 | 30 | 108 | 27 | 1e−05 | RNA dependent DNA replication |
| 335 | 2.438833644 | down | 0.002052115 | 0.016384686 | BE820646 | 471 | 66 | 151 | 43 | 7e−34 | electron transport |
| 336 | 2.440271524 | down | 0.007963355 | 0.040387982 | BM527005 | 570 | 12 | 26 | 46 | 0.67 | |
| 337 | 2.441271178 | down | 0.009120108 | 0.044218063 | U63726.1 | | | | | | gamma glutamyl hydrolase |
| 338 | 2.441931198 | down | 0.00182953 | 0.015184457 | BU545678 | 587 | 19 | 57 | 33 | 0.11 | |
| 339 | 2.446808447 | down | 0.001524275 | 0.013477687 | BU577878 | 480 | 80 | 107 | 74 | 3e−45 | electron transport |
| 340 | 2.447287609 | down | 0.006461887 | 0.03521416 | AW707191 | 555 | 80 | 155 | 51 | 2e−38 | regulation of transcription |
| 341 | 2.450604531 | down | 0.000489044 | 0.006602617 | BI971984 | 553 | 138 | 184 | 75 | 2e−76 | |
| 342 | 2.452345932 | down | 0.000191481 | 0.003743432 | BU550583 | 650 | 54 | 126 | 42 | 3e−18 | |
| 343 | 2.453428965 | down | 0.000494077 | 0.006639592 | AW101990 | 442 | 50 | 74 | 67 | 5e−26 | protein amino acid phosphorylation |
| 344 | 2.454621929 | down | 0.000051 | 0.001917433 | BE058386 | 434 | 27 | 68 | 39 | 4e−04 | |
| 345 | 2.455012187 | down | 0.002047337 | 0.016384686 | BI497844 | 335 | 18 | 30 | 60 | 4e−04 | |
| 346 | 2.455114443 | down | 0.000026 | 0.001380871 | BM522407 | 559 | 52 | 90 | 57 | 2e−27 | carbohydrate metabolism |
| 347 | 2.455162414 | down | 0.001277182 | 0.012079438 | CD405547 | 603 | 36 | 82 | 43 | 1e−07 | regulation of transcription\, DNA- |
| | | | | | | | | | | | dependent |
| 348 | 2.455689154 | down | 0.000477908 | 0.006498931 | BQ298588 | 354 | 14 | 28 | 50 | 1.9 | |
| 349 | 2.45593101 | down | 0.00378891 | 0.024432723 | AW620343 | 381 | 83 | 126 | 65 | 4e−43 | |
| 350 | 2.457923947 | down | 0.005376371 | 0.031031278 | BE821395 | | | | | | |
| 351 | 2.459838507 | down | 0.006007056 | 0.033489836 | AW423990 | 442 | 89 | 105 | 84 | 9e−52 | electron transport |
| 352 | 2.468170208 | down | 0.001061741 | 0.010805285 | BU546941 | 643 | 39 | 99 | 39 | 1e−12 | aging |
| 353 | 2.468241341 | down | 0.0000875 | 0.002488219 | CD410115 | 653 | 88 | 141 | 62 | 6e−41 | ciliary/flagellar motility |
| 354 | 2.471014111 | down | 0.007994895 | 0.0404792 | BG043694 | 533 | 41 | 76 | 53 | 2e−16 | |
| 355 | 2.471735459 | down | 0.000361427 | 0.005454491 | CD417424 | 696 | 128 | 167 | 76 | 1e−70 | photosynthesis |
| 356 | 2.472737501 | down | 0.006140402 | 0.033980443 | CD393748 | 543 | 15 | 54 | 27 | 3.0 | |
| 357 | 2.472773185 | down | 0.003108969 | 0.021484613 | CD415914 | 589 | 58 | 62 | 93 | 1e−29 | |
| 358 | 2.475410167 | down | 0.001082305 | 0.01092202 | BG508509 | | | | | | |
| 359 | 2.476391256 | down | 0.000790229 | 0.00894856 | AF301590.1 | | | | | | Cytosolic glutamine synthetase |
| | | | | | | | | | | | GSbeta 1 |
| 360 | 2.479297024 | down | 0.001931506 | 0.015725472 | BG509587 | 471 | 9 | 16 | 56 | 1.8 | metal ion transport |
| 361 | 2.479316218 | down | 0.000410589 | 0.005881143 | BF067733 | 404 | 82 | 119 | 68 | 6e−42 | photosynthesis |
| 362 | 2.480258744 | down | 0.002937355 | 0.020697952 | AI959966 | 570 | 52 | 124 | 41 | 8e−20 | regulation of transcription |
| 363 | 2.480332751 | down | 0.0000803 | 0.002395372 | BI893662 | 510 | 115 | 142 | 80 | 3e−65 | proteolysis and peptidolysis |
| 364 | 2.481622536 | down | 0.0000262 | 0.001380871 | CD401349 | 257 | 12 | 47 | 25 | 7.2 | |
| 365 | 2.482693922 | down | 0.000287392 | 0.004770501 | CD404800 | 376 | 27 | 35 | 77 | 8e−11 | photosynthesis light harvesting |
| 366 | 2.483552348 | down | 0.003711883 | 0.024076349 | BU091400 | 473 | 21 | 74 | 28 | 0.27 | |
| 367 | 2.485387634 | down | 0.00245532 | 0.018430333 | AW101957 | 340 | 50 | 139 | 35 | 3e−15 | |
| 368 | 2.48876946 | down | 0.002694793 | 0.019594425 | BG508375 | 261 | 18 | 52 | 34 | 0.65 | electron transport |
| 369 | 2.489972787 | down | 0.003759731 | 0.024303137 | CD391105 | 663 | 86 | 136 | 63 | 4e−44 | regulation of transcription |
| 370 | 2.497082099 | down | 0.001890807 | 0.015498109 | BG238507 | 522 | 68 | 82 | 82 | 4e−35 | circadian rhythm |
| 371 | 2.497083485 | down | 0.005334414 | 0.030888032 | BU546067 | 552 | 62 | 128 | 48 | 5e−32 | very-long-chain fatty acid metabolism |
| 372 | 2.500626957 | down | 0.001558213 | 0.013681257 | AW309766 | 626 | 90 | 170 | 52 | 1e−42 | protein amino acid phosphorylation |
| 373 | 2.501009331 | down | 0.003458149 | 0.022981413 | AW132856 | 524 | 34 | 68 | 50 | 4e−09 | |
| 374 | 2.504460361 | down | 0.007339834 | 0.038206547 | BI700686 | 597 | 27 | 33 | 81 | 9e−10 | |
| 375 | 2.50551793 | down | 0.001279501 | 0.012092252 | BG507801 | 397 | 15 | 50 | 30 | 3.4 | ubiquitin-dependent protein |
| | | | | | | | | | | | catabolism |
| 376 | 2.506047581 | down | 0.000268211 | 0.004579435 | AW432320 | 472 | 44 | 122 | 36 | 3e−10 | regulation of transcription\, DNA- |
| | | | | | | | | | | | dependent |
| 377 | 2.50688213 | down | 0.001183769 | 0.011532317 | BI941775 | 503 | 12 | 16 | 75 | 0.11 | |
| 378 | 2.507311313 | down | 0.00064123 | 0.007855504 | CD406073 | 678 | 133 | 167 | 79 | 1e−71 | |
| 379 | 2.509393736 | down | 0.001553547 | 0.013653061 | CD403514 | 595 | 49 | 101 | 48 | 2e−22 | proton transport |
| 380 | 2.513434956 | down | 0.006370622 | 0.034915429 | CD391061 | 628 | 20 | 37 | 54 | 5e−05 | aromatic compound metabolism |
| 381 | 2.517446876 | down | 0.001083647 | 0.010927299 | BI701059 | 350 | 16 | 22 | 72 | 0.001 | electron transport |
| 382 | 2.517683638 | down | 0.0000248 | 0.001362306 | AW350270 | 761 | 70 | 150 | 46 | 8e−30 | cell death |
| 383 | 2.520534345 | down | 0.004098497 | 0.025727778 | CD412604 | 653 | 52 | 106 | 49 | 2e−25 | metabolism |
| 384 | 2.520930211 | down | 0.0000342 | 0.00156033 | AW597896 | 240 | 51 | 55 | 92 | 4e−26 | potassium ion transport |
| 385 | 2.521290896 | down | 0.000771082 | 0.008834441 | AW152996 | 425 | 20 | 57 | 35 | 1.8 | |
| 386 | 2.522203624 | down | 0.000471674 | 0.006449127 | AF363021.1 | | | | | | cytosolic glutamine synthetase beta 2 |
| 387 | 2.522564888 | down | 0.007120099 | 0.037502506 | BE823084 | 618 | 100 | 128 | 78 | 8e−57 | |
| 388 | 2.522884377 | down | 0.002025011 | 0.016271899 | BU546442 | 489 | 54 | 105 | 51 | 2e−26 | |
| 389 | 2.524279783 | down | 0.000513178 | 0.006794322 | BQ298667 | 382 | 13 | 26 | 50 | 0.80 | |
| 390 | 2.525378101 | down | 0.009355325 | 0.045010384 | BM521030 | 444 | 31 | 99 | 31 | 7e−06 | |
| 391 | 2.529059754 | down | 0.000832894 | 0.009242976 | CD418036 | 640 | 47 | 86 | 54 | 4e−18 | |
| 392 | 2.529995232 | down | 0.01022152 | 0.04778596 | AW597585 | 418 | 41 | 94 | 43 | 8e−12 | |
| 393 | 2.530695959 | down | 0.003231671 | 0.022036995 | BE608750 | 439 | 14 | 36 | 38 | 0.39 | transport |
| 394 | 2.530867449 | down | 0.00000566 | 0.000820095 | AW704103 | 371 | 10 | 13 | 76 | 2.2 | regulation of transcription\, DNA- |
| | | | | | | | | | | | dependent |
| 395 | 2.531371382 | down | 0.002932979 | 0.020685994 | CD409241 | 446 | 12 | 33 | 36 | 1.5 | |
| 396 | 2.532068408 | down | 0.000922834 | 0.009842516 | BU760651 | 444 | 16 | 50 | 32 | 0.89 | electron transport |
| 397 | 2.53366636 | down | 0.006149105 | 0.034018597 | AI736757 | 428 | 15 | 40 | 37 | 0.13 | |
| 398 | 2.538105365 | down | 0.001440964 | 0.012981399 | CD409882 | 626 | 35 | 102 | 34 | 1e−07 | |
| 399 | 2.540515255 | down | 0.003675768 | 0.023941182 | BI468833 | 492 | 88 | 117 | 75 | 1e−45 | |
| 400 | 2.543329997 | down | 0.005717104 | 0.032361649 | AB030493.1 | | | | | | thiamin biosynthetic enzyme |
| 401 | 2.54351594 | down | 0.006324461 | 0.034748508 | BI470070 | 325 | 18 | 28 | 64 | 6e−07 | |
| 402 | 2.545279525 | down | 0.000432313 | 0.006085711 | CA818882 | 395 | 17 | 43 | 39 | 0.40 | brassinosteroid biosynthesis |
| 403 | 2.545762362 | down | 0.000214304 | 0.004024387 | CD396298 | 694 | 162 | 172 | 94 | 2e−91 | chlorophyll a/b-binding protein |
| | | | | | | | | | | | precursor, photosynthesis light |
| | | | | | | | | | | | harvesting in photosystem II |
| 404 | 2.545983621 | down | 0.002272708 | 0.017494614 | AW707132 | 610 | 82 | 172 | 47 | 5e−40 | |
| 405 | 2.548006245 | down | 0.001396854 | 0.01275893 | BM177649 | 523 | 72 | 80 | 90 | 1e−39 | photosynthesis light harvesting |
| 406 | 2.551153223 | down | 0.001495559 | 0.013314543 | AI959849 | 503 | 14 | 51 | 27 | 2.6 | |
| 407 | 2.551187962 | down | 0.002442045 | 0.01836367 | BE609741 | 266 | 33 | 71 | 46 | 2e−10 | protein amino acid phosphorylation |
| 408 | 2.552375095 | down | 0.000282339 | 0.004728362 | BF067657 | 427 | 87 | 119 | 73 | 1e−45 | ciliary/flagellar motility |
| 409 | 2.552606761 | down | 0.004518124 | 0.027624302 | BE658352 | 377 | 20 | 46 | 43 | 0.005 | |
| 410 | 2.552956902 | down | 0.0000614 | 0.002089098 | BQ454274 | 439 | 13 | 36 | 36 | 0.67 | |
| 411 | 2.554712393 | down | 0.002825607 | 0.020217347 | BI967466 | 474 | 16 | 42 | 38 | 0.16 | |
| 412 | 2.554970438 | down | 0.010589716 | 0.048861821 | CD392652 | 381 | 12 | 33 | 36 | 4.0 | |
| 413 | 2.559195692 | down | 0.001576582 | 0.01377504 | BG157524 | 404 | 27 | 96 | 28 | 0.010 | DNA replication |
| 414 | 2.559740605 | down | 0.006429073 | 0.035077336 | CD403334 | 657 | 112 | 140 | 80 | 3e−66 | response to dessication |
| 415 | 2.560818803 | down | 0.002396453 | 0.018104077 | AI431227 | 341 | 36 | 102 | 35 | 2e−07 | electron transport |
| 416 | 2.561386756 | down | 0.001436899 | 0.012957929 | BE822397 | 618 | 109 | 179 | 60 | 4e−64 | |
| 417 | 2.563523853 | down | 0.00686339 | 0.036587237 | CD393360 | 528 | 17 | 23 | 73 | 1e−04 | glycolysis |
| 418 | 2.565809074 | down | 0.003833144 | 0.024650347 | AW201116 | 365 | 54 | 122 | 44 | 8e−24 | |
| 419 | 2.567149555 | down | 0.009162046 | 0.044381351 | BE800180 | 491 | 11 | 32 | 34 | 1.1 | |
| 420 | 2.56951058 | down | 0.002186188 | 0.017127947 | BM886486 | 528 | 110 | 158 | 69 | 2e−57 | |
| 421 | 2.571272412 | down | 0.000602442 | 0.007558567 | BE348221 | 491 | 73 | 82 | 89 | 4e−37 | |
| 422 | 2.572305673 | down | 0.006844544 | 0.036523007 | BE659472 | 778 | 67 | 180 | 37 | 3e−18 | |
| 423 | 2.572576102 | down | 0.001227184 | 0.011766305 | BQ611623 | 617 | 48 | 114 | 42 | 3e−12 | |
| 424 | 2.574998394 | down | 0.000111074 | 0.002790978 | CD396712 | 462 | 17 | 44 | 38 | 2.2 | |
| 425 | 2.577551626 | down | 0.010401334 | 0.048288791 | CF808286 | | | | | | |
| 426 | 2.579501672 | down | 0.0000123 | 0.001057791 | AF243371.1 | | | | | | glutathione S-transferase GST 16 |
| 427 | 2.584039081 | down | 0.000104582 | 0.002705473 | AW348689 | 573 | 23 | 34 | 67 | 6e−07 | ciliary/flagellar motility |
| 428 | 2.586214591 | down | 0.001815957 | 0.015120416 | BG511652 | 329 | 31 | 56 | 55 | 1e−13 | |
| 429 | 2.591912038 | down | 0.001581989 | 0.013800155 | BG551594 | 318 | 11 | 27 | 40 | 5.4 | cell growth and/or maintenance |
| 430 | 2.592752195 | down | 0.00078554 | 0.008932069 | BE022626 | | | | | | |
| 431 | 2.593511161 | down | 0.005828112 | 0.032787704 | U13182.1 | | | | | | hypothetical LOC547849 |
| 432 | 2.603585092 | down | 0.005155831 | 0.030184979 | CD413817 | 524 | 60 | 90 | 66 | 6e−29 | protein amino acid phosphorylation |
| 433 | 2.604776535 | down | 0.001596405 | 0.013877324 | BQ080393 | 347 | 101 | 115 | 87 | 6e−55 | DNA repair |
| 434 | 2.604952437 | down | 0.006552499 | 0.03551891 | CF806299 | 650 | 92 | 210 | 43 | 4e−42 | ethylene mediated signaling pathway |
| 435 | 2.613062889 | down | 0.009419956 | 0.045205695 | BI425980 | 517 | 80 | 131 | 61 | 1e−37 | regulation of transcription\, DNA- |
| | | | | | | | | | | | dependent |
| 436 | 2.613683287 | down | 0.000199575 | 0.003839804 | AW132172 | 365 | 16 | 45 | 35 | 0.42 | |
| 437 | 2.613723586 | down | 0.000274448 | 0.004645862 | AW310578 | 262 | 11 | 19 | 57 | 0.29 | |
| 438 | 2.615888903 | down | 0.002745796 | 0.019838999 | BE820525 | 761 | 76 | 95 | 80 | 2e−42 | |
| 439 | 2.620124774 | down | 0.000235949 | 0.004260065 | CD416691 | 698 | 34 | 55 | 61 | 1e−10 | G-protein coupled receptor protein |
| | | | | | | | | | | | signaling pathway |
| 440 | 2.622239262 | down | 0.005093333 | 0.029939148 | BM144029 | 533 | 36 | 95 | 37 | 9e−10 | |
| 441 | 2.628477796 | down | 0.000821815 | 0.009148195 | BF598781 | 431 | 15 | 43 | 34 | 0.65 | |
| 442 | 2.633606508 | down | 0.001667495 | 0.014280775 | BM732034 | 542 | 57 | 75 | 76 | 2e−26 | |
| 443 | 2.63447462 | down | 0.005656519 | 0.032126158 | CF806416 | 652 | 64 | 139 | 46 | 5e−27 | |
| 444 | 2.640459518 | down | 0.003940696 | 0.025084053 | BE330206 | 503 | 24 | 52 | 46 | 2e−04 | regulation of transcription\, DNA- |
| | | | | | | | | | | | dependent |
| 445 | 2.643527082 | down | 0.000682497 | 0.008158544 | CD417087 | 677 | 18 | 51 | 35 | 5.8 | regulation of transcription\, DNA- |
| | | | | | | | | | | | dependent |
| 446 | 2.643898743 | down | 0.003903267 | 0.024935249 | BM527963 | 534 | 12 | 30 | 40 | 1.7 | |
| 447 | 2.644424939 | down | 0.000274772 | 0.004647151 | BE657843 | 309 | 9 | 20 | 45 | 4.2 | |
| 448 | 2.645656742 | down | 0.00113082 | 0.011221282 | Y15076.1 | | | | | | ferredoxin thioredoxin reductase |
| | | | | | | | | | | | precursor |
| 449 | 2.646490686 | down | 0.0000197 | 0.001250712 | CA819306 | 251 | 11 | 23 | 47 | 5.6 | regulation of transcription\, DNA- |
| | | | | | | | | | | | dependent |
| 450 | 2.647256974 | down | 0.002887397 | 0.020497672 | BF426150 | 276 | 10 | 21 | 47 | 0.49 | |
| 451 | 2.648276532 | down | 0.009879629 | 0.046673295 | AB062754.1 | | | | | | ferritin |
| 452 | 2.650922005 | down | 0.001793243 | 0.015012473 | BU547563 | 650 | 81 | 101 | 80 | 4e−37 | transport |
| 453 | 2.650951633 | down | 0.007601084 | 0.039121464 | BQ612268 | 382 | 13 | 29 | 44 | 0.002 | dicarboxylic acid transport |
| 454 | 2.651200979 | down | 0.0000902 | 0.002526814 | BI469914 | 300 | 12 | 29 | 41 | 3.2 | |
| 455 | 2.656915044 | down | 0.00000895 | 0.00091778 | BQ273338 | 590 | 16 | 33 | 48 | 5e−04 | |
| 456 | 2.657789717 | down | 0.001669369 | 0.014292417 | BG510539 | 287 | 14 | 50 | 28 | 1.4 | |
| 457 | 2.658580453 | down | 0.0000612 | 0.002087031 | BG653611 | 499 | 70 | 143 | 48 | 3e−32 | resistance protein MG13 |
| 458 | 2.658704975 | down | 0.001999048 | 0.01612752 | BM893052 | 558 | 88 | 104 | 84 | 2e−47 | |
| 459 | 2.660151037 | down | 0.000402944 | 0.005826398 | BQ453283 | 435 | 52 | 57 | 91 | 5e−25 | carbohydrate metabolism |
| 460 | 2.661621331 | down | 0.000115644 | 0.002848619 | BE058386 | 434 | 27 | 68 | 39 | 4e−04 | |
| 461 | 2.664030542 | down | 0.004595427 | 0.027924454 | AW310932 | 424 | 11 | 28 | 39 | 3.0 | |
| 462 | 2.664260241 | down | 0.000105068 | 0.00270768 | BU764573 | 457 | 64 | 90 | 71 | 9e−34 | amino acid transport |
| 463 | 2.665034229 | down | 0.009730219 | 0.046185493 | AW348397 | 790 | 110 | 212 | 51 | 1e−59 | lipoxygenase |
| 464 | 2.666547648 | down | 0.001504915 | 0.013363047 | BE820324 | 383 | 13 | 35 | 37 | 3.1 | proteolysis and peptidolysis |
| 465 | 2.67134064 | down | 0.001932904 | 0.015730044 | BE057084 | 277 | 23 | 53 | 43 | 0.012 | protein complex assembly |
| 466 | 2.679202732 | down | 0.005525796 | 0.03160971 | CD417321 | 357 | 28 | 43 | 65 | 8e−12 | |
| 467 | 2.680309267 | down | 0.000165567 | 0.003455957 | BM178538 | 393 | 16 | 46 | 34 | 0.14 | regulation of translational initiation |
| 468 | 2.682014387 | down | 0.001211271 | 0.01168224 | AW349565 | 697 | 106 | 173 | 61 | 7e−63 | protein amino acid phosphorylation |
| 469 | 2.684738219 | down | 0.001183401 | 0.011531719 | BQ627821 | 578 | 95 | 178 | 53 | 3e−50 | carbohydrate metabolism |
| 470 | 2.686412809 | down | 0.001085526 | 0.010937846 | CD418039 | | | | | | |
| 471 | 2.688591441 | down | 0.009715295 | 0.046134466 | AW201851 | | | | | | |
| 472 | 2.690034534 | down | 0.010739892 | 0.049354954 | BE060005 | 455 | 19 | 28 | 67 | 1e−04 | |
| 473 | 2.690162571 | down | 0.0000861 | 0.002468444 | CF808919 | 441 | 51 | 128 | 39 | 3e−19 | |
| 474 | 2.691398796 | down | 0.006562431 | 0.035538056 | BM107802 | 394 | 16 | 34 | 47 | 0.047 | ethylene mediated signaling pathway |
| 475 | 2.691516948 | down | 0.000491126 | 0.006623602 | BI967918 | 694 | 22 | 55 | 40 | 0.003 | G-protein coupled receptor protein |
| | | | | | | | | | | | signaling pathway |
| 476 | 2.696373949 | down | 0.00204309 | 0.016361246 | BM270590 | 369 | 13 | 33 | 39 | 0.74 | |
| 477 | 2.700819346 | down | 0.008710213 | 0.042894205 | AI930773 | 478 | 58 | 105 | 55 | 3e−23 | |
| 478 | 2.70262647 | down | 0.00253165 | 0.018804491 | CD395831 | 668 | 75 | 91 | 82 | 3e−38 | |
| 479 | 2.70663696 | down | 0.00143258 | 0.012933387 | CD403179 | 591 | 51 | 104 | 49 | 5e−18 | proteolysis and peptidolysis |
| 480 | 2.708550533 | down | 0.001822646 | 0.01515072 | BU548610 | 657 | 41 | 96 | 42 | 1e−12 | DNA repair |
| 481 | 2.710076065 | down | 0.00000561 | 0.000820095 | CF806642 | 273 | 7 | 17 | 41 | 2.4 | |
| 482 | 2.711498952 | down | 0.003037269 | 0.021150805 | BE820477 | 468 | 89 | 111 | 80 | 2e−50 | multidrug transport |
| 483 | 2.714336198 | down | 0.000392794 | 0.005733243 | BU578491 | 568 | 154 | 189 | 81 | 1e−88 | protein amino acid phosphorylation |
| 484 | 2.724459882 | down | 0.002770834 | 0.019943008 | BI426052 | 370 | 42 | 44 | 95 | 9e−20 | microtubule-based process |
| 485 | 2.726210558 | down | 0.000373781 | 0.005554873 | BE657917 | 581 | 36 | 131 | 27 | 2e−10 | defense response |
| 486 | 2.726231605 | down | 0.000899822 | 0.009683106 | AF243379.1 | | | | | | glutathione S-transferase GST 24 |
| 487 | 2.730188762 | down | 0.005590431 | 0.031860376 | CA935007 | 334 | 12 | 33 | 36 | 4.2 | |
| 488 | 2.731025983 | down | 0.006294027 | 0.034631878 | BM521030 | 444 | 31 | 99 | 31 | 7e−06 | |
| 489 | 2.733761513 | down | 0.000758595 | 0.008750017 | CA936869 | 540 | 62 | 125 | 49 | 2e−27 | protein amino acid phosphorylation |
| 490 | 2.73790549 | down | 0.0000442 | 0.00177615 | CD416100 | 689 | 87 | 144 | 60 | 8e−45 | protein amino acid phosphorylation |
| 491 | 2.74188226 | down | 0.000343974 | 0.005300377 | BG238497 | 572 | 109 | 188 | 57 | 1e−56 | proteolysis and peptidolysis |
| 492 | 2.743018238 | down | 0.000478869 | 0.006504934 | BI968049 | 315 | 10 | 21 | 47 | 1.9 | |
| 493 | 2.743988679 | down | 0.001176535 | 0.011509964 | BF596725 | 378 | 35 | 42 | 83 | 4e−15 | branched chain family amino acid |
| | | | | | | | | | | | biosynthesis |
| 494 | 2.744937929 | down | 0.003429801 | 0.022878507 | BM093715 | 441 | 19 | 26 | 73 | 9e−06 | |
| 495 | 2.749471031 | down | 0.002718869 | 0.01969837 | BI945123 | | | | | | |
| 496 | 2.759154064 | down | 0.000136173 | 0.003091825 | BU550583 | 650 | 54 | 126 | 42 | 3e−18 | |
| 497 | 2.759704981 | down | 0.0000161 | 0.0011509 | BE059671 | 557 | 112 | 168 | 66 | 3e−60 | |
| 498 | 2.765845017 | down | 0.000849355 | 0.009364113 | BQ094713 | 489 | 11 | 27 | 40 | 4.2 | |
| 499 | 2.773188995 | down | 0.000295272 | 0.004830959 | BI969908 | 774 | 80 | 100 | 80 | 8e−44 | jasmonic acid mediated signaling |
| | | | | | | | | | | | pathway (jasmonic acid/ethylene |
| | | | | | | | | | | | dependent systemic resistance) |
| 500 | 2.773340956 | down | 0.000116053 | 0.002848619 | BI968242 | 611 | 73 | 115 | 63 | 3e−40 | aromatic compound metabolism |
| 501 | 2.775085598 | down | 0.000252386 | 0.004420006 | BF219543 | 582 | 69 | 96 | 71 | 2e−34 | |
| 502 | 2.77636551 | down | 0.006060316 | 0.03367101 | BE820594 | 671 | 42 | 69 | 60 | 1e−17 | metal ion transport |
| 503 | 2.780406174 | down | 0.000250135 | 0.004407052 | CF807373 | 265 | 14 | 50 | 28 | 1.4 | 24 kDa seed coat protein |
| 504 | 2.789743468 | down | 0.009002265 | 0.043816151 | CA783795 | 433 | 40 | 97 | 41 | 4e−11 | |
| 505 | 2.79983936 | down | 0.001222718 | 0.011750461 | BG508816 | 204 | 13 | 40 | 32 | 9.2 | |
| 506 | 2.801866742 | down | 0.003475196 | 0.023049908 | BF009132 | 447 | 55 | 87 | 63 | 3e−30 | embryonic development (sensu |
| | | | | | | | | | | | Magnoliophyta) |
| 507 | 2.80324494 | down | 0.000858551 | 0.009412277 | BU762337 | 635 | 116 | 206 | 56 | 9e−61 | |
| 508 | 2.806526361 | down | 0.005568154 | 0.031776506 | BE819915 | 866 | 147 | 218 | 67 | 2e−79 | aldehyde dehydrogenase family 7 |
| | | | | | | | | | | | member A1, proline biosynthesis |
| 509 | 2.80859339 | down | 0.0000124 | 0.001058667 | BI469429 | 566 | 55 | 121 | 45 | 2e−26 | electron transport |
| 510 | 2.809913295 | down | 0.005477387 | 0.031435222 | BU084208 | 596 | 12 | 24 | 50 | 4.7 | apoptosis |
| 511 | 2.817795727 | down | 0.005522061 | 0.031600001 | BQ299038 | 579 | 39 | 59 | 66 | 1e−20 | transport |
| 512 | 2.819142076 | down | 0.001107938 | 0.011072154 | BG155556 | 460 | 58 | 73 | 79 | 7e−31 | glutamate dehydrogenase 1, electron |
| | | | | | | | | | | | transport |
| 513 | 2.820462943 | down | 0.00556282 | 0.031751127 | BQ253025 | 594 | 12 | 31 | 38 | 3.5 | |
| 514 | 2.820532309 | down | 0.00318846 | 0.021854343 | BM886413 | 553 | 55 | 93 | 59 | 8e−25 | |
| 515 | 2.823387261 | down | 0.002802391 | 0.020085585 | BI967474 | 644 | 78 | 110 | 70 | 2e−43 | chitin catabolism |
| 516 | 2.82668618 | down | 0.001226391 | 0.011763568 | BU762161 | 580 | 19 | 50 | 38 | 0.53 | defense response |
| 517 | 2.839997163 | down | 0.00064847 | 0.007909869 | BG509076 | 311 | 16 | 33 | 48 | 0.026 | steroid metabolism |
| 518 | 2.841113117 | down | 0.001341245 | 0.012447465 | BI700897 | 396 | 19 | 52 | 36 | 0.30 | |
| 519 | 2.841641373 | down | 0.000120409 | 0.00290697 | BG652873 | 463 | 64 | 91 | 70 | 8e−32 | regulation of transcription |
| 520 | 2.843664447 | down | 0.00981715 | 0.04645393 | CD394552 | 709 | 51 | 148 | 34 | 1e−16 | |
| 521 | 2.846933626 | down | 0.001616086 | 0.013986986 | BF423911 | 472 | 53 | 62 | 85 | 5e−24 | |
| 522 | 2.849256015 | down | 0.002414328 | 0.018202554 | CF920436 | 662 | 80 | 120 | 66 | 2e−45 | |
| 523 | 2.852886268 | down | 0.001904023 | 0.015572044 | BG511748 | 330 | 16 | 18 | 88 | 0.004 | |
| 524 | 2.852953439 | down | 0.003875424 | 0.024845783 | AW707000 | 557 | 28 | 101 | 27 | 2.4 | |
| 525 | 2.854443909 | down | 0.000974841 | 0.010188292 | CF806515 | 512 | 86 | 112 | 76 | 2e−47 | |
| 526 | 2.857438669 | down | 0.005758535 | 0.032517871 | BE024072 | 313 | 10 | 16 | 62 | 4.2 | |
| 527 | 2.857574167 | down | 0.003248316 | 0.022118419 | AW307490 | 243 | 13 | 29 | 44 | 1.1 | cysteine proteinase |
| 528 | 2.857985286 | down | 0.008422727 | 0.041984062 | AW307490 | 243 | 13 | 29 | 44 | 1.1 | cysteine proteinase |
| 529 | 2.859419071 | down | 0.001578129 | 0.013785354 | BU549671 | 685 | 25 | 48 | 52 | 1e−05 | blue light signaling pathway |
| 530 | 2.862052018 | down | 0.000208458 | 0.003952455 | CA936289 | 567 | 146 | 189 | 77 | 1e−80 | protein amino acid phosphorylation |
| 531 | 2.865641468 | down | 0.002394627 | 0.018097547 | CA852377 | 386 | 12 | 28 | 42 | 0.85 | |
| 532 | 2.868880768 | down | 0.002962671 | 0.020821732 | AI416885 | 506 | 43 | 49 | 87 | 1e−20 | |
| 533 | 2.869259241 | down | 0.001416964 | 0.012856283 | BE822399 | 741 | 75 | 136 | 55 | 4e−41 | proteolysis and peptidolysis |
| 534 | 2.873851564 | down | 0.000136081 | 0.003091825 | CD392179 | 625 | 64 | 107 | 59 | 1e−31 | |
| 535 | 2.875428363 | down | 0.00111659 | 0.011134517 | CD398677 | 448 | 44 | 88 | 50 | 2e−18 | response to pathogenic bacteria |
| | | | | | | | | | | | (incompatible interaction) |
| 536 | 2.876773248 | down | 0.000473057 | 0.006463333 | AW133007 | 515 | 103 | 156 | 66 | 4e−48 | regulation of transcription\, DNA- |
| | | | | | | | | | | | dependent |
| 537 | 2.880360265 | down | 0.000809673 | 0.009077561 | BE802446 | 428 | 100 | 134 | 74 | 1e−56 | |
| 538 | 2.880493465 | down | 0.000792051 | 0.008954675 | AI856378 | 417 | 30 | 41 | 73 | 4e−09 | |
| 539 | 2.882040949 | down | 0.000519573 | 0.006847663 | CD394361 | 717 | 165 | 194 | 85 | 1e−93 | photosynthesis light harvesting |
| 540 | 2.882478104 | down | 0.010297005 | 0.047970217 | BI968685 | 792 | 63 | 155 | 40 | 1e−20 | auxin mediated signaling |
| 541 | 2.885253707 | down | 0.000243922 | 0.004340089 | BE658807 | 382 | 14 | 38 | 36 | 1.1 | |
| 542 | 2.885319664 | down | 0.005057379 | 0.029792994 | BI892728 | 421 | 16 | 51 | 31 | 2.3 | neuropeptide signaling pathway |
| 543 | 2.88554391 | down | 0.0000383 | 0.001670558 | BI967718 | 653 | 31 | 104 | 29 | 0.22 | |
| 544 | 2.887840527 | down | 0.010185479 | 0.047657249 | CD414547 | 512 | 30 | 63 | 47 | 4e−10 | |
| 545 | 2.889415196 | down | 0.001298871 | 0.012232284 | BQ610611 | 354 | 20 | 29 | 68 | 6e−07 | response to stress |
| 546 | 2.890661013 | down | 0.00021892 | 0.004072519 | CD409224 | 392 | 25 | 90 | 27 | 2.6 | electron transport |
| 547 | 2.893820299 | down | 0.000658158 | 0.007974312 | CF807372 | 748 | 139 | 254 | 54 | 1e−67 | |
| 548 | 2.898399003 | down | 0.000775665 | 0.008870381 | CD414210 | 593 | 33 | 69 | 47 | 8e−10 | |
| 549 | 2.900395003 | down | 0.002211393 | 0.017224619 | AW348132 | 721 | 139 | 238 | 58 | 2e−72 | lipid metabolism |
| 550 | 2.900553378 | down | 0.000113825 | 0.002831633 | BG789835 | 305 | 43 | 61 | 70 | 6e−18 | response to wounding |
| 551 | 2.904854061 | down | 0.000423828 | 0.006010626 | AW733209 | 356 | 38 | 70 | 54 | 1e−16 | |
| 552 | 2.907883516 | down | 0.0000774 | 0.002341624 | BQ299018 | 293 | 12 | 27 | 44 | 3.3 | |
| 553 | 2.913380853 | down | 0.000961292 | 0.010091554 | CF807507 | 457 | 36 | 46 | 78 | 3e−17 | fatty acid alpha-oxidation |
| 554 | 2.913916147 | down | 0.000123707 | 0.002950604 | BU083449 | 363 | 11 | 34 | 32 | 2.7 | |
| 555 | 2.914162573 | down | 0.00089461 | 0.009658402 | CD392418 | 640 | 40 | 84 | 47 | 3e−12 | |
| 556 | 2.920013803 | down | 0.0000405 | 0.00172704 | AI960155 | 475 | 57 | 60 | 95 | 4e−27 | |
| 557 | 2.923663409 | down | 0.0000508 | 0.001913604 | BE659664 | 446 | 11 | 20 | 55 | 7.7 | plasmid partitioning (sensu Bacteria) |
| 558 | 2.92473771 | down | 0.00061062 | 0.007620386 | BQ454285 | 355 | 12 | 46 | 26 | 5.6 | response to mechanical stimulus |
| 559 | 2.927553644 | down | 0.003649273 | 0.023855365 | AW307490 | 243 | 13 | 29 | 44 | 1.1 | cysteine proteinase |
| 560 | 2.930610648 | down | 0.00124879 | 0.011896764 | BE057404 | 393 | 14 | 22 | 63 | 0.028 | regulation of transcription\, DNA- |
| | | | | | | | | | | | dependent |
| 561 | 2.933888591 | down | 0.000304201 | 0.004918258 | AI736533 | 436 | 10 | 21 | 47 | 2.5 | multidrug transport |
| 562 | 2.937849968 | down | 0.000502219 | 0.006689253 | BE822092 | 651 | 64 | 146 | 43 | 2e−24 | |
| 563 | 2.938103168 | down | 0.000284613 | 0.004741119 | CD394332 | 681 | 90 | 107 | 84 | 1e−49 | |
| 564 | 2.93856323 | down | 0.004647551 | 0.028136644 | BG508624 | 364 | 15 | 50 | 30 | 0.92 | |
| 565 | 2.944070433 | down | 0.002352589 | 0.017880436 | CD404982 | 394 | 11 | 29 | 37 | 4.4 | |
| 566 | 2.948457441 | down | 0.000996269 | 0.010351836 | BE658686 | | | | | | |
| 567 | 2.948461084 | down | 0.010312919 | 0.04802651 | BQ296811 | 574 | 26 | 43 | 60 | 3e−08 | |
| 568 | 2.949909373 | down | 0.004600722 | 0.027947601 | AW310967 | 551 | 25 | 83 | 30 | 6e−07 | ciliary/flagellar motility |
| 569 | 2.951740494 | down | 0.001321504 | 0.012342421 | CD398324 | 582 | 35 | 61 | 57 | 7e−14 | |
| 570 | 2.956740677 | down | 0.006772361 | 0.036304606 | CD391191 | 593 | 39 | 93 | 41 | 2e−12 | |
| 571 | 2.959551528 | down | 0.000316336 | 0.00504181 | BI968254 | 765 | 118 | 155 | 76 | 2e−69 | photosynthetic water oxidation |
| 572 | 2.973969426 | down | 0.010620798 | 0.048993219 | CD416993 | 637 | 98 | 176 | 55 | 4e−43 | |
| 573 | 2.980881707 | down | 0.00143289 | 0.012933387 | CD398655 | 611 | 72 | 88 | 81 | 9e−39 | GMP biosynthesis |
| 574 | 2.984648589 | down | 0.001391592 | 0.012729416 | AW306851 | 187 | 36 | 59 | 61 | 6e−17 | protein amino acid phosphorylation |
| 575 | 2.987807838 | down | 0.006303876 | 0.034670855 | AW348511 | 738 | 116 | 184 | 63 | 1e−64 | |
| 576 | 2.993590423 | down | 0.000214532 | 0.004025893 | CD418598 | 606 | 52 | 110 | 47 | 4e−19 | |
| 577 | 2.993781694 | down | 0.010863567 | 0.049753102 | BG511716 | 286 | 15 | 46 | 32 | 3.2 | regulation of transcription\, DNA- |
| | | | | | | | | | | | dependent |
| 578 | 2.996035712 | down | 0.000280647 | 0.004710512 | AW132799 | 638 | 153 | 213 | 71 | 3e−84 | photosynthesis |
| 579 | 3.000221781 | down | 0.000143847 | 0.003182607 | BI943612 | 625 | 22 | 44 | 50 | 4e−05 | photosynthesis |
| 580 | 3.00430351 | down | 0.002085071 | 0.016553083 | BE659788 | 437 | 15 | 41 | 36 | 0.30 | |
| 581 | 3.005127093 | down | 0.00020946 | 0.003963086 | AI974100 | 273 | 14 | 32 | 43 | 0.84 | protein amino acid phosphorylation |
| 582 | 3.005589212 | down | 0.0000969 | 0.00260019 | BI973828 | 533 | 25 | 71 | 35 | 5e−05 | |
| 583 | 3.00577259 | down | 0.000351537 | 0.005368458 | CD395689 | 327 | 11 | 20 | 55 | 1.8 | electron transport |
| 584 | 3.005852156 | down | 0.00760786 | 0.039144566 | BE211206 | 577 | 96 | 192 | 50 | 7e−41 | |
| 585 | 3.00879189 | down | 0.001176578 | 0.011509964 | AB086392.1 | | | | | | simillar to Arabidopsis thaliana sucrose-proton |
| | | | | | | | | | | | symporter protein |
| 586 | 3.011495778 | down | 0.001710774 | 0.014532306 | AW152996 | 425 | 20 | 57 | 35 | 1.8 | |
| 587 | 3.012306684 | down | 0.004254854 | 0.026435759 | BE824219 | 610 | 10 | 32 | 31 | 6.3 | metabolism |
| 588 | 3.012473306 | down | 0.000250161 | 0.004407052 | BE022465 | 248 | 12 | 28 | 42 | 3.1 | regulation of transcription\, DNA- |
| | | | | | | | | | | | dependent |
| 589 | 3.012540124 | down | 0.003977562 | 0.025229683 | CD408197 | 376 | 19 | 56 | 33 | 2.2 | transport |
| 590 | 3.013303354 | down | 0.001257428 | 0.01195272 | CF807038 | 510 | 74 | 164 | 45 | 7e−35 | response to wounding |
| 591 | 3.01374089 | down | 0.002029015 | 0.016287976 | AB000130.1 | | | | | | src2 protein |
| 592 | 3.016547242 | down | 0.001533248 | 0.013515722 | BQ299410 | 582 | 93 | 190 | 48 | 1e−47 | electron transport |
| 593 | 3.020064211 | down | 0.008966823 | 0.043694532 | CD416993 | 637 | 98 | 176 | 55 | 4e−43 | |
| 594 | 3.023704272 | down | 0.0000392 | 0.001692808 | BM178426 | 353 | 14 | 34 | 41 | 0.51 | |
| 595 | 3.027701751 | down | 0.00000154 | 0.000704945 | BU091737 | 673 | 44 | 114 | 38 | 2e−09 | |
| 596 | 3.031928686 | down | 0.000116053 | 0.002848619 | BU579160 | 584 | 14 | 29 | 48 | 0.41 | protein amino acid phosphorylation |
| 597 | 3.032268518 | down | 0.002348907 | 0.017866891 | CA784848 | 729 | 70 | 136 | 51 | 1e−31 | |
| 598 | 3.034196205 | down | 0.000179216 | 0.003632031 | CD406753 | 626 | 43 | 95 | 45 | 4e−13 | |
| 599 | 3.044208258 | down | 0.000918945 | 0.009814079 | CF808678 | 434 | 39 | 72 | 54 | 2e−13 | salinity response |
| 600 | 3.06236175 | down | 0.004070258 | 0.025593195 | BE657635 | 540 | 49 | 88 | 55 | 7e−23 | metabolism |
| 601 | 3.066319425 | down | 0.00752169 | 0.038846746 | BG041895 | 406 | 22 | 66 | 33 | 1.6 | |
| 602 | 3.069194149 | down | 0.001409001 | 0.012820068 | BI941802 | 263 | 13 | 53 | 24 | 4.2 | |
| 603 | 3.070053022 | down | 0.000288513 | 0.004773484 | BM891946 | 563 | 70 | 145 | 48 | 3e−28 | proton transport |
| 604 | 3.076156532 | down | 0.003003684 | 0.02101573 | BM107802 | 394 | 16 | 34 | 47 | 0.047 | ethylene mediated signaling pathway |
| 605 | 3.077374542 | down | 0.007411215 | 0.038455708 | BM527363 | 530 | 17 | 37 | 45 | 0.018 | |
| 606 | 3.078581751 | down | 0.000456352 | 0.006321149 | BQ740268 | 599 | 96 | 140 | 68 | 1e−47 | regulation of transcription\, DNA- |
| | | | | | | | | | | | dependent |
| 607 | 3.087382816 | down | 0.001275097 | 0.012065788 | AW781053 | 552 | 92 | 167 | 55 | 3e−46 | lipid metabolism |
| 608 | 3.09602638 | down | 0.000907717 | 0.00972747 | BM732633 | 551 | 14 | 51 | 27 | 3.1 | Hydroxyproline-rich glycoprotein |
| | | | | | | | | | | | (hrgp) |
| 609 | 3.106067633 | down | 0.002949545 | 0.020754874 | CF921631 | 321 | 29 | 39 | 74 | 3e−10 | metabolism |
| 610 | 3.10686292 | down | 0.000764353 | 0.008777221 | CA803090 | | | | | | |
| 611 | 3.107041658 | down | 0.0000731 | 0.002273082 | BM142992 | 553 | 66 | 115 | 57 | 3e−34 | |
| 612 | 3.116088445 | down | 0.000278066 | 0.004686009 | CD392332 | 604 | 34 | 38 | 89 | 2e−12 | |
| 613 | 3.117322837 | down | 0.006643098 | 0.035854998 | CA938720 | 446 | 17 | 43 | 39 | 0.004 | |
| 614 | 3.119257483 | down | 0.000359901 | 0.005443424 | BQ298945 | 427 | 31 | 54 | 57 | 3e−09 | |
| 615 | 3.119966575 | down | 0.000618378 | 0.007684068 | CA820071 | 427 | 25 | 44 | 56 | 3e−11 | glutathione transferase, aromatic |
| | | | | | | | | | | | amino acid family metabolism |
| 616 | 3.125561862 | down | 0.0000329 | 0.001543897 | BG789835 | 305 | 43 | 61 | 70 | 6e−18 | response to wounding |
| 617 | 3.130617296 | down | 0.000182203 | 0.003660995 | AW351155 | 348 | 19 | 41 | 46 | 2e−04 | |
| 618 | 3.133097754 | down | 0.000492698 | 0.006632463 | BG511038 | 347 | 19 | 58 | 32 | 0.84 | |
| 619 | 3.139129084 | down | 0.001232143 | 0.011795833 | BI967912 | 769 | 65 | 191 | 34 | 5e−14 | |
| 620 | 3.13954004 | down | 0.009603158 | 0.045810167 | CD402881 | 699 | 67 | 100 | 67 | 4e−32 | regulation of transcription\, DNA- |
| | | | | | | | | | | | dependent |
| 621 | 3.14142841 | down | 0.001199447 | 0.011624836 | CD409882 | 626 | 35 | 102 | 34 | 1e−07 | |
| 622 | 3.152169592 | down | 0.001249012 | 0.011896764 | BG650381 | 477 | 49 | 183 | 26 | 3e−07 | |
| 623 | 3.157364942 | down | 0.000495537 | 0.006642601 | AW706721 | 171 | 12 | 30 | 40 | 1.5 | |
| 624 | 3.157510089 | down | 0.000123543 | 0.002950604 | BE659214 | 541 | 17 | 36 | 47 | 0.014 | |
| 625 | 3.161871879 | down | 0.000455173 | 0.006314252 | BG651854 | 494 | 40 | 75 | 53 | 1e−14 | G beta-like protein |
| 626 | 3.161947737 | down | 0.0000419 | 0.001750979 | BQ297653 | 528 | 19 | 29 | 65 | 2e−05 | protein amino acid phosphorylation |
| 627 | 3.167330033 | down | 0.005091599 | 0.029939148 | AW278868 | 502 | 44 | 52 | 84 | 2e−20 | protein folding |
| 628 | 3.179161304 | down | 0.001289596 | 0.012175418 | CF808477 | 559 | 99 | 162 | 61 | 3e−60 | |
| 629 | 3.182722876 | down | 0.000256749 | 0.004464791 | BQ627520 | 341 | 10 | 23 | 43 | 1.1 | |
| 630 | 3.183491315 | down | 0.001757605 | 0.014826322 | CD398227 | 449 | 15 | 54 | 27 | 0.084 | |
| 631 | 3.184880159 | down | 0.000178856 | 0.003632031 | BQ628871 | 463 | 25 | 84 | 29 | 2.9 | protein amino acid phosphorylation |
| 632 | 3.187040928 | down | 0.001225518 | 0.011763568 | BI425372 | 429 | 21 | 59 | 35 | 1e−04 | protein amino acid phosphorylation |
| 633 | 3.193464992 | down | 0.000213938 | 0.004019515 | AW568786 | 448 | 59 | 105 | 56 | 6e−26 | |
| 634 | 3.212870176 | down | 0.000297834 | 0.004855972 | BU762280 | 397 | 13 | 34 | 38 | 0.68 | |
| 635 | 3.213438404 | down | 0.006616084 | 0.035160507 | AW349120 | 670 | 59 | 132 | 44 | 4e−14 | regulation of transcription\, DNA- |
| | | | | | | | | | | | dependent |
| 636 | 3.21577993 | down | 0.000712175 | 0.008392157 | AI855922 | 436 | 23 | 61 | 37 | 0.012 | |
| 637 | 3.218673543 | down | 0.009976706 | 0.046926879 | BF596737 | 620 | 17 | 32 | 53 | 0.041 | |
| 638 | 3.223238493 | down | 0.001147356 | 0.011327203 | CF808931 | 684 | 132 | 216 | 61 | 9e−78 | GmGST mRNA for geraniol-inducible |
| | | | | | | | | | | | glutathione S-transferase |
| 639 | 3.223681137 | down | 0.0000949 | 0.002566638 | BE821100 | 675 | 38 | 66 | 57 | 8e−15 | zinc ion transport |
| 640 | 3.22944116 | down | 0.00210335 | 0.016669915 | BM307614 | 468 | 42 | 116 | 36 | 5e−10 | electron transport |
| 641 | 3.231641594 | down | 0.007980506 | 0.040449904 | BU545606 | 645 | 52 | 122 | 42 | 5e−17 | protein amino acid phosphorylation |
| 642 | 3.232524222 | down | 0.000755247 | 0.008716958 | BM887472 | 551 | 100 | 140 | 71 | 2e−54 | |
| 643 | 3.232716597 | down | 0.000763116 | 0.008775139 | BE657646 | 415 | 26 | 30 | 86 | 1e−10 | |
| 644 | 3.239812832 | down | 0.00048465 | 0.006571609 | AB062754.1 | | | | | | ferritin |
| 645 | 3.248452145 | down | 0.00000816 | 0.000899886 | AI988142 | 647 | 51 | 112 | 45 | 8e−25 | protein amino acid phosphorylation |
| 646 | 3.249989435 | down | 0.000132292 | 0.003049042 | BE820284 | 740 | 73 | 180 | 40 | 8e−25 | |
| 647 | 3.253885402 | down | 0.000223805 | 0.004122529 | AI900506 | 413 | 26 | 38 | 68 | 2e−09 | |
| 648 | 3.26165059 | down | 0.001192175 | 0.011587206 | CD393442 | 709 | 85 | 102 | 83 | 1e−43 | |
| 649 | 3.264021765 | down | 0.001352847 | 0.012515001 | AF279267.1 | | | | | | 4-coumarate:coenzyme A ligase |
| 650 | 3.264788278 | down | 0.00025843 | 0.004485737 | BQ612355 | 403 | 81 | 107 | 75 | 3e−47 | oligopeptide transport |
| 651 | 3.27360037 | down | 0.000263131 | 0.00452565 | BI321576 | 586 | 33 | 53 | 62 | 9e−17 | |
| 652 | 3.275640217 | down | 0.001898175 | 0.01553312 | CK605562 | 713 | 32 | 68 | 47 | 1e−12 | metal ion transport |
| 653 | 3.276124438 | down | 0.00000234 | 0.000707618 | BE800639 | 413 | 71 | 136 | 52 | 8e−41 | |
| 654 | 3.27985395 | down | 0.002688602 | 0.019572105 | BI470523 | 250 | 15 | 45 | 33 | 2.4 | |
| 655 | 3.286564536 | down | 0.001306359 | 0.012266724 | BG511457 | 384 | 10 | 37 | 27 | 0.82 | |
| 656 | 3.302958984 | down | 0.002292445 | 0.017583206 | BQ454119 | 239 | 12 | 29 | 41 | 1.9 | |
| 657 | 3.308063396 | down | 0.003450371 | 0.022945942 | BG550873 | 445 | 37 | 67 | 55 | 5e−15 | |
| 658 | 3.318495721 | down | 0.0000821 | 0.002420756 | BG509161 | 318 | 19 | 67 | 28 | 0.38 | transport |
| 659 | 3.319557144 | down | 0.000146555 | 0.003216456 | BI967193 | 737 | 57 | 150 | 38 | 5e−16 | |
| 660 | 3.324840889 | down | 0.00666584 | 0.035926193 | AI855972 | | | | | | |
| 661 | 3.326304459 | down | 0.000106702 | 0.00273398 | BQ612383 | 437 | 16 | 43 | 37 | 0.52 | |
| 662 | 3.331338535 | down | 0.00282167 | 0.020196821 | BG239335 | 422 | 14 | 40 | 35 | 0.28 | |
| 663 | 3.341026735 | down | 0.001895011 | 0.015519037 | BG154860 | 352 | 16 | 39 | 41 | 0.64 | RNA dependent DNA replication |
| 664 | 3.357528631 | down | 0.001458024 | 0.013079445 | CA820021 | 346 | 20 | 30 | 66 | 5e−07 | |
| 665 | 3.359050721 | down | 0.002985823 | 0.020936523 | BQ296111 | 574 | 93 | 207 | 44 | 8e−39 | positive regulation of transcription |
| 666 | 3.359611146 | down | 0.0000209 | 0.001283285 | BI497646 | 392 | 81 | 129 | 62 | 6e−44 | protein amino acid phosphorylation |
| 667 | 3.366805492 | down | 0.000139753 | 0.003145136 | BQ741049 | 580 | 136 | 190 | 71 | 7e−81 | metabolism |
| 668 | 3.368519976 | down | 0.005797241 | 0.03268306 | BE209739 | 478 | 27 | 61 | 44 | 9e−10 | |
| 669 | 3.375220057 | down | 0.0000438 | 0.001772209 | BE021748 | 415 | 10 | 32 | 31 | 3.8 | |
| 670 | 3.377399639 | down | 0.000508563 | 0.006752082 | BE612253 | 351 | 18 | 21 | 85 | 6e−04 | |
| 671 | 3.380792185 | down | 0.004530348 | 0.027685542 | BM526911 | 432 | 49 | 68 | 72 | 2e−24 | response to pest/pathogen/parasite |
| 672 | 3.381121954 | down | 0.000161934 | 0.003408507 | BG510710 | | | | | | |
| 673 | 3.38467428 | down | 0.000175808 | 0.003591302 | BU544028 | 586 | 26 | 65 | 40 | 1e−05 | |
| 674 | 3.386288791 | down | 0.005731275 | 0.032394518 | BU084823 | 330 | 21 | 32 | 65 | 5e−07 | |
| 675 | 3.393647534 | down | 0.0000138 | 0.001103751 | CD409471 | 460 | 51 | 105 | 48 | 2e−23 | protein amino acid phosphorylation |
| 676 | 3.40651415 | down | 0.005510706 | 0.031563833 | BE659352 | 442 | 14 | 42 | 33 | 0.11 | proteolysis and peptidolysis |
| 677 | 3.407661119 | down | 0.00087033 | 0.009475313 | CD414577 | 652 | 103 | 168 | 61 | 5e−62 | |
| 678 | 3.415592272 | down | 0.0000245 | 0.001353826 | BE822234 | 674 | 102 | 173 | 58 | 7e−57 | proteolysis and peptidolysis |
| 679 | 3.416403291 | down | 0.002071521 | 0.016501099 | CF806578 | 512 | 15 | 17 | 88 | 0.013 | |
| 680 | 3.417678697 | down | 0.000635114 | 0.007817744 | CD406104 | 723 | 89 | 127 | 70 | 2e−42 | photosynthesis |
| 681 | 3.422910905 | down | 0.0000767 | 0.002334034 | BI967564 | 595 | 18 | 48 | 37 | 0.32 | |
| 682 | 3.442338977 | down | 0.000602236 | 0.007558567 | CD406104 | 723 | 89 | 127 | 70 | 2e−42 | photosynthesis |
| 683 | 3.451487706 | down | 0.006044749 | 0.033594441 | BM140209 | 177 | 14 | 44 | 31 | 2.5 | regulation of transcription |
| 684 | 3.461280268 | down | 0.003117816 | 0.021525975 | BI946245 | 333 | 48 | 68 | 70 | 1e−25 | |
| 685 | 3.475695354 | down | 0.0000326 | 0.001543897 | BG789835 | 305 | 43 | 61 | 70 | 6e−18 | response to wounding |
| 686 | 3.489370674 | down | 0.002176056 | 0.017100571 | CD411677 | 623 | 45 | 81 | 55 | 4e−18 | proton transport |
| 687 | 3.499721731 | down | 0.001941429 | 0.015778935 | BI970208 | 770 | 25 | 46 | 54 | 5e−08 | metal ion transport |
| 688 | 3.511498878 | down | 0.00109268 | 0.010971717 | CA819497 | 214 | 11 | 27 | 40 | 1.9 | |
| 689 | 3.525412642 | down | 0.000379458 | 0.005608036 | CD402384 | 321 | 13 | 30 | 43 | 0.83 | DNA metabolism |
| 690 | 3.528162444 | down | 0.000571513 | 0.007277213 | AW307590 | | | | | | |
| 691 | 3.533014879 | down | 0.000748596 | 0.008672578 | CA852403 | 268 | 12 | 34 | 35 | 7.2 | transport |
| 692 | 3.537516589 | down | 0.007126545 | 0.037522099 | L01447.1 | | | | | | G-box binding factor |
| 693 | 3.538870024 | down | 0.000943385 | 0.009980826 | BU765669 | 430 | 55 | 67 | 82 | 2e−30 | |
| 694 | 3.557451905 | down | 0.00000206 | 0.000707618 | BE347229 | 358 | 23 | 45 | 51 | 1e−07 | proteolysis and peptidolysis |
| 695 | 3.560671578 | down | 0.001521925 | 0.013466403 | BQ454088 | 389 | 14 | 56 | 25 | 5.6 | |
| 696 | 3.564378864 | down | 0.003572339 | 0.023520716 | CD412016 | 668 | 19 | 43 | 44 | 0.67 | |
| 697 | 3.565079147 | down | 0.000934936 | 0.009925676 | AI856145 | 264 | 53 | 69 | 76 | 4e−26 | |
| 698 | 3.58116219 | down | 0.001788492 | 0.01498604 | BE805053 | 602 | 65 | 151 | 43 | 1e−27 | G-protein coupled receptor protein |
| | | | | | | | | | | | signaling pathway |
| 699 | 3.584395671 | down | 0.000763573 | 0.008775139 | AI965698 | 461 | 11 | 21 | 52 | 8.3 | |
| 700 | 3.586390662 | down | 0.008808908 | 0.043204888 | AW310948 | 692 | 117 | 171 | 68 | 9e−62 | trehalose biosynthesis |
| 701 | 3.595606881 | down | 0.005621741 | 0.031990343 | BE023603 | 428 | 68 | 132 | 51 | 2e−36 | |
| 702 | 3.609392349 | down | 0.0000238 | 0.00133979 | CD408731 | 550 | 83 | 138 | 60 | 9e−40 | transport |
| 703 | 3.614648165 | down | 0.0000455 | 0.001800696 | CD414114 | 650 | 87 | 135 | 64 | 9e−51 | |
| 704 | 3.616613645 | down | 0.010826901 | 0.049634762 | BI970404 | 796 | 66 | 102 | 64 | 2e−28 | |
| 705 | 3.617768307 | down | 0.0000324 | 0.001543897 | CF808173 | 612 | 46 | 83 | 55 | 5e−21 | protein amino acid phosphorylation |
| 706 | 3.618215718 | down | 0.00614836 | 0.034018597 | AI938444 | 352 | 56 | 67 | 83 | 4e−26 | ATP-dependent proteolysis |
| 707 | 3.628863802 | down | 0.0000143 | 0.001117991 | BM107998 | 712 | 41 | 88 | 46 | 2e−15 | apoptosis |
| 708 | 3.629798062 | down | 0.001175853 | 0.011508861 | BE804769 | 522 | 68 | 121 | 56 | 6e−32 | |
| 709 | 3.630148931 | down | 0.000581255 | 0.00736139 | BI426378 | 224 | 9 | 20 | 45 | 9.4 | cell wall biosynthesis (sensu |
| | | | | | | | | | | | Magnoliophyta) |
| 710 | 3.631495654 | down | 0.0000176 | 0.001209655 | CD418138 | 473 | 50 | 101 | 49 | 2e−21 | |
| 711 | 3.637311523 | down | 0.001069361 | 0.010859292 | BQ740743 | 457 | 15 | 59 | 25 | 2.1 | |
| 712 | 3.64893541 | down | 0.000114418 | 0.002833761 | BG725838 | 515 | 28 | 51 | 54 | 3e−10 | aromatic compound metabolism |
| 713 | 3.651425148 | down | 0.00152861 | 0.013486401 | BQ296620 | 536 | 104 | 177 | 58 | 3e−48 | |
| 714 | 3.652228641 | down | 0.000121714 | 0.002929089 | CF807644 | 596 | 122 | 159 | 76 | 4e−70 | |
| 715 | 3.659000821 | down | 0.000100019 | 0.002647286 | BQ452657 | 588 | 69 | 72 | 95 | 1e−34 | biological_process unknown |
| 716 | 3.670011898 | down | 0.00000656 | 0.00085619 | BQ081093 | 342 | 36 | 88 | 40 | 4e−06 | |
| 717 | 3.672030338 | down | 0.000164779 | 0.003443322 | CF806235 | 637 | 84 | 107 | 78 | 7e−55 | regulation of transcription |
| 718 | 3.67203162 | down | 0.000100579 | 0.002653677 | AF195028.1 | | | | | | plasma membrane Ca2+-ATPase |
| 719 | 3.674714806 | down | 0.003346421 | 0.022557756 | BG507829 | 399 | 31 | 57 | 54 | 7e−12 | |
| 720 | 3.675785079 | down | 0.000432877 | 0.006089987 | CF808027 | 533 | 127 | 179 | 70 | 6e−68 | |
| 721 | 3.677611188 | down | 0.0000726 | 0.002264259 | AW706013 | 362 | 19 | 33 | 57 | 0.24 | |
| 722 | 3.680804565 | down | 0.003661372 | 0.023884642 | BQ576552 | 499 | 23 | 51 | 45 | 9e−06 | |
| 723 | 3.689482231 | down | 0.0000848 | 0.002461966 | CD416724 | 705 | 104 | 183 | 56 | 1e−50 | |
| 724 | 3.693693264 | down | 0.0000436 | 0.001771311 | BE824094 | 711 | 116 | 157 | 73 | 1e−63 | protein amino acid phosphorylation |
| 725 | 3.693987647 | down | 0.0000864 | 0.002469742 | BU548684 | 687 | 94 | 136 | 69 | 3e−50 | SferH-2 mRNA for ferritin, iron ion |
| | | | | | | | | | | | transport |
| 726 | 3.697739614 | down | 0.003252042 | 0.022131776 | BE658601 | 475 | 18 | 34 | 52 | 0.004 | |
| 727 | 3.703495756 | down | 0.0000822 | 0.002420756 | BU964540 | 550 | 32 | 63 | 50 | 2e−09 | regulation of transcription |
| 728 | 3.705703609 | down | 0.003289834 | 0.02231629 | BU548241 | 553 | 18 | 64 | 28 | 1.4 | |
| 729 | 3.70780396 | down | 0.000317662 | 0.005048874 | CD413509 | 718 | 15 | 70 | 21 | 0.34 | |
| 730 | 3.708359952 | down | 0.000582196 | 0.007368341 | BM528250 | 555 | 81 | 145 | 55 | 6e−38 | signal transduction |
| 731 | 3.719960067 | down | 0.000566876 | 0.007230407 | BE347412 | 468 | 89 | 145 | 61 | 6e−47 | sulfate transport |
| 732 | 3.720721093 | down | 0.001562391 | 0.013696465 | CB063482 | 417 | 11 | 20 | 55 | 6.7 | DNA repair |
| 733 | 3.735211855 | down | 0.00013516 | 0.003079293 | AI966345 | 614 | 65 | 147 | 44 | 2e−27 | |
| 734 | 3.739285473 | down | 0.000614774 | 0.007653815 | BE657686 | 610 | 71 | 108 | 65 | 7e−36 | |
| 735 | 3.740903663 | down | 0.004695521 | 0.028327037 | CF809068 | 600 | 30 | 47 | 63 | 3e−14 | |
| 736 | 3.742689351 | down | 0.0000502 | 0.001899206 | CD396554 | 736 | 171 | 189 | 90 | e−105 | photosynthesis light harvesting in |
| | | | | | | | | | | | photosystem I |
| 737 | 3.749050309 | down | 0.0000736 | 0.00228073 | BI470435 | 534 | 45 | 101 | 44 | 3e−20 | protein amino acid phosphorylation |
| 738 | 3.752057801 | down | 0.000142754 | 0.003175131 | BQ299050 | 205 | 8 | 14 | 57 | 7.0 | |
| 739 | 3.752315031 | down | 0.0000614 | 0.002089098 | CF809241 | 573 | 55 | 115 | 47 | 6e−21 | |
| 740 | 3.765225748 | down | 0.0000577 | 0.002027667 | BG651885 | 390 | 35 | 120 | 29 | 8e−05 | regulation of transcription |
| 741 | 3.769910357 | down | 0.000695947 | 0.00827089 | CA799365 | 408 | 59 | 92 | 64 | 1e−26 | |
| 742 | 3.773966305 | down | 0.000452534 | 0.006290321 | BQ740743 | 457 | 15 | 59 | 25 | 2.1 | |
| 743 | 3.781657613 | down | 0.000134916 | 0.003077454 | BQ454004 | 592 | 67 | 67 | 100 | 6e−32 | ubiquitin-dependent protein |
| | | | | | | | | | | | catabolism |
| 744 | 3.790482453 | down | 0.005634532 | 0.032038896 | CA783731 | 740 | 106 | 203 | 52 | 8e−52 | |
| 745 | 3.790694175 | down | 0.000014 | 0.001108626 | CD413105 | 667 | 81 | 107 | 75 | 1e−45 | protein amino acid phosphorylation |
| 746 | 3.805857684 | down | 0.0000378 | 0.001657189 | BM521488 | 357 | 12 | 21 | 57 | 1.5 | |
| 747 | 3.814948935 | down | 0.007712014 | 0.03951501 | CF809068 | 600 | 30 | 47 | 63 | 3e−14 | |
| 748 | 3.818213208 | down | 0.000369226 | 0.005502921 | CD411163 | 691 | 90 | 123 | 73 | 3e−50 | |
| 749 | 3.819709568 | down | 0.000485319 | 0.006578306 | CA802451 | 277 | 14 | 53 | 26 | 4.2 | |
| 750 | 3.819947775 | down | 0.000666539 | 0.00802343 | BQ628641 | 531 | 54 | 105 | 51 | 2e−27 | |
| 751 | 3.828808351 | down | 0.000832599 | 0.009242976 | AW153021 | 384 | 14 | 29 | 48 | 0.48 | response to pathogenic bacteria |
| 752 | 3.835335382 | down | 0.000998013 | 0.010364238 | BI967568 | 547 | 26 | 40 | 65 | 8e−12 | |
| 753 | 3.850654183 | down | 0.001040708 | 0.010675244 | BM525300 | 495 | 52 | 133 | 39 | 9e−19 | |
| 754 | 3.861951858 | down | 0.000500706 | 0.0066762 | CD401157 | 619 | 27 | 33 | 81 | 3e−09 | |
| 755 | 3.864143547 | down | 0.000734524 | 0.008574917 | BE657686 | 610 | 71 | 108 | 65 | 7e−36 | |
| 756 | 3.872328691 | down | 0.005374724 | 0.031031278 | BE022458 | 204 | 11 | 23 | 47 | 3.2 | transport |
| 757 | 3.877691599 | down | 0.0000745 | 0.002292577 | BQ610796 | 186 | 8 | 13 | 61 | 3.2 | response to UV |
| 758 | 3.894602034 | down | 0.0000571 | 0.002012438 | BU548684 | 687 | 94 | 136 | 69 | 3e−50 | SferH-2 mRNA for ferritin, iron ion |
| | | | | | | | | | | | transport |
| 759 | 3.903717694 | down | 0.000228546 | 0.004173076 | BI969674 | 743 | 87 | 174 | 50 | 7e−37 | |
| 760 | 3.912115705 | down | 0.000069 | 0.002191837 | BE800257 | 588 | 43 | 97 | 44 | 3e−18 | carbohydrate metabolism |
| 761 | 3.916104869 | down | 0.000127915 | 0.00299962 | BI321901 | 479 | 108 | 158 | 68 | 5e−61 | DNA repair |
| 762 | 3.923840768 | down | 0.003690176 | 0.023995811 | BE611977 | 438 | 22 | 46 | 47 | 0.027 | regulation of transcription\, DNA- |
| | | | | | | | | | | | dependent |
| 763 | 3.950025758 | down | 0.00485563 | 0.028988695 | BE802186 | 462 | 16 | 31 | 51 | 0.031 | regulation of transcription |
| 764 | 3.962614 | down | 0.004522858 | 0.027648756 | BE819852 | 695 | 95 | 156 | 60 | 3e−51 | lipid catabolism |
| 765 | 3.973458015 | down | 0.000185166 | 0.003694813 | CD393939 | 447 | 56 | 70 | 80 | 4e−28 | |
| 766 | 3.986605989 | down | 0.001149666 | 0.011347031 | AW706745 | 163 | 10 | 19 | 52 | 7.3 | |
| 767 | 3.987927972 | down | 0.000516467 | 0.00682168 | CF808698 | 260 | 27 | 40 | 67 | 7e−13 | |
| 768 | 4.002839327 | down | 0.0000897 | 0.002516945 | BG511605 | 255 | 9 | 17 | 52 | 4.2 | |
| 769 | 4.009122426 | down | 0.000515091 | 0.006810063 | CA819552 | | | | | | |
| 770 | 4.01645465 | down | 0.000141266 | 0.003155335 | BE823592 | 644 | 22 | 54 | 40 | 2e−06 | |
| 771 | 4.021149635 | down | 0.000887127 | 0.009603513 | BE822823 | 668 | 18 | 53 | 33 | 1.5 | proteolysis and peptidolysis |
| 772 | 4.027479982 | down | 0.001646081 | 0.01419108 | BU083616 | 575 | 30 | 66 | 45 | 8e−10 | |
| 773 | 4.028715825 | down | 0.00010137 | 0.002664532 | BG507797 | 414 | 18 | 53 | 33 | 1.7 | proton transport |
| 774 | 4.029981697 | down | 0.000904408 | 0.009707165 | BF219523 | 701 | 91 | 131 | 69 | 1e−46 | |
| 775 | 4.034168681 | down | 0.0000156 | 0.001135545 | BG511678 | | | | | | |
| 776 | 4.034297069 | down | 0.0000888 | 0.002506437 | CD401365 | 703 | 64 | 99 | 64 | 2e−29 | |
| 777 | 4.035630845 | down | 0.0000879 | 0.002493933 | BU550006 | 634 | 58 | 97 | 59 | 1e−26 | response to oxidative stress |
| 778 | 4.06561406 | down | 0.000465374 | 0.00638895 | BE821409 | 754 | 60 | 124 | 48 | 2e−30 | |
| 779 | 4.095955206 | down | 0.00000708 | 0.000873658 | CD416827 | 480 | 13 | 34 | 38 | 1.4 | electron transport |
| 780 | 4.105324179 | down | 0.000130669 | 0.00303449 | CA935006 | 262 | 11 | 36 | 30 | 5.5 | |
| 781 | 4.119568829 | down | 0.00000274 | 0.000720842 | CD414114 | 650 | 87 | 135 | 64 | 9e−51 | |
| 782 | 4.129091378 | down | 0.0000178 | 0.001214142 | CD408184 | 700 | 14 | 37 | 37 | 1.2 | response to water deprivation |
| 783 | 4.183341247 | down | 0.000160307 | 0.003381829 | AW350021 | 751 | 143 | 175 | 81 | 5e−82 | |
| 784 | 4.192119051 | down | 0.001631806 | 0.014082611 | BI321552 | 321 | 27 | 70 | 38 | 0.49 | |
| 785 | 4.225054112 | down | 0.000140315 | 0.003147175 | BI945671 | 371 | 13 | 38 | 34 | 2.9 | |
| 786 | 4.228149299 | down | 0.0000677 | 0.00218184 | BU547775 | 634 | 96 | 111 | 86 | 6e−49 | |
| 787 | 4.230172688 | down | 0.001133165 | 0.011231327 | BM270077 | 273 | 24 | 45 | 53 | 4e−09 | |
| 788 | 4.237074631 | down | 0.001253992 | 0.01193212 | AI736267 | 666 | 136 | 222 | 61 | 1e−80 | response to wounding |
| 789 | 4.241073786 | down | 0.0000198 | 0.001254275 | BQ298628 | 563 | 15 | 44 | 34 | 4.2 | |
| 790 | 4.254494232 | down | 0.000732181 | 0.008555532 | AW397177 | 319 | 10 | 20 | 50 | 3.2 | |
| 791 | 4.255821756 | down | 0.000580195 | 0.007350438 | BM270690 | 533 | 13 | 41 | 31 | 6.6 | |
| 792 | 4.257600295 | down | 0.0000819 | 0.002420422 | CD416562 | 612 | 95 | 140 | 67 | 3e−54 | |
| 793 | 4.258412679 | down | 0.000116821 | 0.002860746 | CD405633 | 309 | 28 | 38 | 73 | 3e−10 | G-protein coupled receptor protein |
| | | | | | | | | | | | signaling pathway |
| 794 | 4.271510526 | down | 0.0000338 | 0.001558741 | CD393550 | 318 | 16 | 54 | 29 | 0.29 | |
| 795 | 4.280406169 | down | 0.0000624 | 0.002103258 | BE824431 | 474 | 81 | 112 | 72 | 4e−42 | photosynthesis |
| 796 | 4.280644145 | down | 0.0000463 | 0.001810721 | BU547775 | 634 | 96 | 111 | 86 | 6e−49 | |
| 797 | 4.293552239 | down | 0.003690535 | 0.023995811 | BU548282 | 709 | 92 | 164 | 56 | 1e−44 | |
| 798 | 4.295717132 | down | 0.000428517 | 0.006056654 | AW234479 | 532 | 17 | 48 | 35 | 6.5 | response to stress |
| 799 | 4.308148954 | down | 0.000745377 | 0.00865327 | BI786624 | 516 | 18 | 52 | 34 | 0.55 | |
| 800 | 4.309850099 | down | 0.0000398 | 0.001711403 | BU965521 | 473 | 31 | 57 | 54 | 4e−11 | regulation of transcription |
| 801 | 4.320885402 | down | 0.000398064 | 0.005789087 | BG362937 | 487 | 11 | 15 | 73 | 0.044 | |
| 802 | 4.321600415 | down | 0.010068373 | 0.047232698 | BQ299283 | 586 | 41 | 64 | 64 | 1e−16 | |
| 803 | 4.33244359 | down | 0.0000263 | 0.001384548 | BG237175 | 279 | 16 | 69 | 23 | 0.84 | |
| 804 | 4.352818569 | down | 0.0000212 | 0.001286626 | CD409367 | 714 | 73 | 155 | 47 | 7e−36 | |
| 805 | 4.365879465 | down | 0.0000707 | 0.002230697 | BE058741 | 263 | 14 | 18 | 77 | 0.29 | |
| 806 | 4.391857313 | down | 0.00000418 | 0.000769894 | BM269644 | 594 | 19 | 29 | 65 | 8e−08 | |
| 807 | 4.405186087 | down | 0.000721685 | 0.008467082 | BE821299 | 326 | 40 | 96 | 41 | 7e−21 | |
| 808 | 4.408409843 | down | 0.000034 | 0.001559984 | BG511457 | 384 | 10 | 37 | 27 | 0.82 | |
| 809 | 4.428127269 | down | 0.000198094 | 0.003826966 | AW459996 | 441 | 21 | 72 | 29 | 1.2 | |
| 810 | 4.430366173 | down | 0.003253171 | 0.022135448 | CF921045 | 628 | 48 | 88 | 54 | 1e−20 | protein folding |
| 811 | 4.464086014 | down | 0.000848676 | 0.009361322 | BQ297791 | 289 | 15 | 51 | 29 | 7.0 | |
| 812 | 4.494987711 | down | 0.00000861 | 0.000900892 | AW156348 | 346 | 18 | 59 | 30 | 1.9 | |
| 813 | 4.501165065 | down | 0.00000346 | 0.000753007 | CD395088 | 588 | 50 | 94 | 53 | 8e−16 | |
| 814 | 4.516157503 | down | 0.001149976 | 0.011347113 | BE821378 | 614 | 42 | 106 | 39 | 4e−14 | |
| 815 | 4.536905769 | down | 0.0000667 | 0.002171397 | BI968765 | 775 | 54 | 80 | 67 | 2e−30 | carbohydrate metabolism |
| 816 | 4.538469018 | down | 0.000198984 | 0.003834388 | BU545974 | 587 | 13 | 31 | 41 | 1.2 | gluconeogenesis |
| 817 | 4.547630008 | down | 0.000294353 | 0.004824317 | BG510367 | 361 | 14 | 41 | 34 | 2.6 | |
| 818 | 4.563195635 | down | 0.002279819 | 0.01752567 | U36191.1 | | | | | | lipoxygenase |
| 819 | 4.593513307 | down | 0.00031304 | 0.005016908 | CD394856 | 671 | 130 | 146 | 89 | 5e−74 | carbohydrate metabolism |
| 820 | 4.602773028 | down | 0.0000113 | 0.001006671 | AI736154 | 349 | 10 | 27 | 37 | 9.3 | |
| 821 | 4.616113749 | down | 0.000367848 | 0.005490568 | AW780736 | 350 | 34 | 44 | 77 | 2e−15 | electron transport |
| 822 | 4.617413064 | down | 0.000364539 | 0.005474833 | BF067710 | 571 | 90 | 112 | 80 | 1e−50 | |
| 823 | 4.644152998 | down | 0.000360216 | 0.005444643 | CD396424 | 617 | 29 | 71 | 40 | 0.018 | |
| 824 | 4.697091161 | down | 0.000131256 | 0.003039857 | BE658173 | 577 | 74 | 81 | 91 | 2e−37 | |
| 825 | 4.71119772 | down | 0.000467705 | 0.006413136 | BI945154 | 734 | 14 | 37 | 37 | 0.35 | Sali3-2 |
| 826 | 4.727303836 | down | 0.000803241 | 0.00902153 | BQ785815 | 609 | 64 | 109 | 58 | 3e−25 | |
| 827 | 4.765599609 | down | 0.000543654 | 0.007029569 | BF067710 | 571 | 90 | 112 | 80 | 1e−50 | |
| 828 | 4.765672906 | down | 0.0000235 | 0.001339615 | BI321588 | 581 | 47 | 65 | 72 | 6e−23 | |
| 829 | 4.766301026 | down | 0.003281443 | 0.022283482 | BU549733 | 348 | 20 | 52 | 38 | 0.84 | |
| 830 | 4.798854618 | down | 0.00003 | 0.00147742 | BG237175 | 279 | 16 | 69 | 23 | 0.84 | |
| 831 | 4.800150908 | down | 0.000854731 | 0.009384081 | CD412938 | 625 | 92 | 155 | 59 | 6e−49 | response to wounding |
| 832 | 4.823945984 | down | 0.0000245 | 0.001353826 | AW432965 | 386 | 15 | 52 | 28 | 9.3 | oligosaccharide metabolism |
| 833 | 4.833018881 | down | 0.000381618 | 0.005622468 | BU578286 | 578 | 63 | 85 | 74 | 7e−33 | |
| 834 | 4.849430345 | down | 0.00013124 | 0.003039857 | CF808970 | 495 | 47 | 96 | 48 | 4e−16 | indole-3-acetic acid induced protein |
| | | | | | | | | | | | ARG-2 homolog, response to |
| | | | | | | | | | | | reactive oxygen species |
| 835 | 4.873944265 | down | 0.000177673 | 0.00361419 | CF809250 | 548 | 67 | 177 | 37 | 4e−34 | electron transport |
| 836 | 4.880918462 | down | 0.000464911 | 0.00638895 | AW349564 | 420 | 21 | 31 | 67 | 1e−06 | electron transport |
| 837 | 4.915790836 | down | 0.0000948 | 0.002566638 | BU762030 | 381 | 16 | 31 | 51 | 0.004 | |
| 838 | 4.93282129 | down | 0.00183986 | 0.015223166 | BQ627853 | 429 | 61 | 102 | 59 | 6e−34 | |
| 839 | 4.938358447 | down | 0.00010128 | 0.002664013 | BI427101 | 420 | 11 | 42 | 26 | 2.3 | |
| 840 | 4.948919808 | down | 0.000034 | 0.001558741 | BG237175 | 279 | 16 | 69 | 23 | 0.84 | |
| 841 | 4.972095812 | down | 0.000109599 | 0.002783571 | BE658824 | 362 | 27 | 70 | 38 | 0.001 | |
| 842 | 4.991783217 | down | 0.0000219 | 0.001310321 | BG508418 | 386 | 16 | 47 | 34 | 0.007 | regulation of transcription\, DNA- |
| | | | | | | | | | | | dependent |
| 843 | 5.004766312 | down | 0.0000187 | 0.001230108 | BG508418 | 386 | 16 | 47 | 34 | 0.007 | regulation of transcription\, DNA- |
| | | | | | | | | | | | dependent |
| 844 | 5.037702139 | down | 0.00050107 | 0.006678683 | AW350291 | 698 | 129 | 175 | 73 | 5e−71 | |
| 845 | 5.046265878 | down | 0.0000329 | 0.001543897 | AI960141 | 684 | 71 | 100 | 71 | 4e−34 | |
| 846 | 5.049087423 | down | 0.000157986 | 0.003347889 | AW309932 | 673 | 76 | 87 | 87 | 3e−41 | hypothetical LOC547601 |
| 847 | 5.098775537 | down | 0.000607408 | 0.007590376 | BF599073 | 455 | 91 | 139 | 65 | 8e−51 | |
| 848 | 5.130840148 | down | 0.000327678 | 0.005161333 | BI946236 | 341 | 9 | 30 | 30 | 1.9 | proteolysis and peptidolysis |
| 849 | 5.18571969 | down | 0.0000231 | 0.001328603 | BG650417 | 359 | 21 | 52 | 40 | 0.68 | |
| 850 | 5.200228842 | down | 0.0000863 | 0.002469418 | BG508042 | 315 | 12 | 43 | 27 | 2.4 | |
| 851 | 5.255681298 | down | 0.00000582 | 0.000829921 | AW704921 | 411 | 47 | 91 | 51 | 1e−18 | |
| 852 | 5.272307166 | down | 0.000041 | 0.001734775 | CA819873 | 367 | 12 | 30 | 40 | 2.8 | protein biosynthesis |
| 853 | 5.280183623 | down | 0.000149481 | 0.003255682 | BQ786355 | 150 | 11 | 32 | 34 | 9.2 | |
| 854 | 5.296048032 | down | 0.000131661 | 0.003040011 | BF066553 | 364 | 97 | 121 | 80 | 4e−52 | terpenoid biosynthesis |
| 855 | 5.319644672 | down | 0.0000988 | 0.002632822 | BI700784 | 369 | 13 | 16 | 81 | 0.20 | response to oxidative stress |
| 856 | 5.320281527 | down | 0.000155547 | 0.003326183 | AW349880 | 601 | 39 | 106 | 36 | 9e−10 | regulation of transcription\, DNA- |
| | | | | | | | | | | | dependent |
| 857 | 5.33119303 | down | 0.000089 | 0.002506437 | BI975081 | 566 | 32 | 47 | 68 | 1e−12 | |
| 858 | 5.333052022 | down | 0.000166177 | 0.003464842 | CF808678 | 434 | 39 | 72 | 54 | 2e−13 | salinity response |
| 859 | 5.364896535 | down | 0.00219171 | 0.017153349 | CF809203 | 407 | 75 | 100 | 75 | 8e−40 | Win gene encoding wound-induced |
| | | | | | | | | | | | protein, response to virus |
| 860 | 5.387475211 | down | 0.0000936 | 0.00255512 | AI855886 | 281 | 16 | 20 | 80 | 0.004 | carbohydrate metabolism |
| 861 | 5.422080773 | down | 0.0000111 | 0.000993386 | CF805964 | 559 | 54 | 90 | 60 | 3e−24 | |
| 862 | 5.434260993 | down | 0.000167349 | 0.003479645 | CA853434 | 350 | 34 | 64 | 53 | 1e−11 | transport |
| 863 | 5.456939012 | down | 0.0000452 | 0.001793484 | BM732305 | 420 | 15 | 33 | 45 | 0.042 | DNA repair |
| 864 | 5.5347149 | down | 0.00030996 | 0.004986674 | BE348124 | 484 | 20 | 69 | 28 | 0.28 | electron transport |
| 865 | 5.670176408 | down | 0.0000567 | 0.002008532 | BU547499 | 634 | 71 | 131 | 54 | 1e−35 | |
| 866 | 5.700793287 | down | 0.000358279 | 0.005427896 | U89693.1 | | | | | | Sali3-2 |
| 867 | 5.7840182 | down | 0.00000261 | 0.000712455 | CD416587 | 602 | 16 | 39 | 41 | 2.1 | |
| 868 | 5.802962369 | down | 0.000182117 | 0.003660995 | BI700784 | 369 | 13 | 16 | 81 | 0.20 | response to oxidative stress |
| 869 | 5.846792872 | down | 0.0000116 | 0.001020193 | AI939190 | 527 | 77 | 114 | 67 | 3e−41 | metabolism |
| 870 | 5.853736166 | down | 0.000131178 | 0.003039857 | BI975081 | 566 | 32 | 47 | 68 | 1e−12 | |
| 871 | 5.856316582 | down | 0.000432116 | 0.006085212 | BI424099 | 547 | 63 | 169 | 37 | 1e−26 | metabolism |
| 872 | 5.888210855 | down | 0.00000473 | 0.000799682 | CD394432 | 699 | 93 | 113 | 82 | 2e−51 | |
| 873 | 5.926712891 | down | 0.000940658 | 0.009972351 | AW394524 | 400 | 12 | 39 | 30 | 2.6 | metabolism |
| 874 | 5.94979846 | down | 0.000579824 | 0.007348219 | BQ785967 | 476 | 19 | 64 | 29 | 0.015 | |
| 875 | 6.006490631 | down | 0.000150456 | 0.003269358 | AI856145 | 264 | 53 | 69 | 76 | 4e−26 | |
| 876 | 6.146871238 | down | 0.0000233 | 0.001330585 | AI856254 | 559 | 8 | 16 | 50 | 7.1 | electron transport |
| 877 | 6.247868447 | down | 0.001629041 | 0.01406352 | BI945193 | | | | | | |
| 878 | 6.286396908 | down | 0.0000673 | 0.002177084 | BE824281 | 666 | 95 | 114 | 83 | 2e−53 | |
| 879 | 6.387760656 | down | 0.000148046 | 0.003239438 | CA851238 | 318 | 15 | 36 | 41 | 0.84 | regulation of transcription\, DNA- |
| | | | | | | | | | | | dependent |
| 880 | 6.444005172 | down | 0.0000212 | 0.001286626 | CD393428 | 449 | 113 | 143 | 79 | 6e−71 | |
| 881 | 6.487833314 | down | 0.0000279 | 0.001441369 | CD408184 | 700 | 14 | 37 | 37 | 1.2 | response to water deprivation |
| 882 | 6.490940139 | down | 0.00000617 | 0.000832282 | CD410679 | 626 | 12 | 28 | 42 | 5.1 | |
| 883 | 6.633627838 | down | 0.0000264 | 0.001388554 | CA802722 | 265 | 8 | 16 | 50 | 9.4 | |
| 884 | 6.655577709 | down | 0.00049406 | 0.006639592 | U26457.1 | | | | | | |
| 885 | 6.661720242 | down | 0.0000751 | 0.002304812 | BM307457 | 319 | 16 | 41 | 39 | 0.005 | regulation of transcription |
| 886 | 6.671277186 | down | 0.0002692 | 0.00458997 | BQ628654 | 457 | 52 | 81 | 64 | 6e−27 | electron transport |
| 887 | 6.688875259 | down | 0.000149251 | 0.003254069 | BQ786098 | 423 | 11 | 26 | 42 | 2.3 | |
| 888 | 6.716664254 | down | 0.000227651 | 0.004162802 | BG510367 | 361 | 14 | 41 | 34 | 2.6 | |
| 889 | 6.748518028 | down | 0.0000521 | 0.001943186 | BI700522 | 420 | 126 | 139 | 90 | 1e−71 | hypothetical LOC547601 |
| 890 | 6.865326132 | down | 0.002510136 | 0.018687311 | AW202185 | 388 | 19 | 80 | 23 | 3.2 | |
| 891 | 7.014681065 | down | 0.000353098 | 0.005373204 | BQ612000 | 271 | 12 | 36 | 33 | 1.4 | DNA recombination |
| 892 | 7.019147897 | down | 0.0000255 | 0.001374317 | CD403709 | 451 | 16 | 26 | 61 | 4.6 | |
| 893 | 7.041243398 | down | 0.000198988 | 0.003834388 | BU547403 | 441 | 18 | 54 | 33 | 0.69 | proteolysis and peptidolysis |
| 894 | 7.206743725 | down | 0.0000301 | 0.001477923 | BE821995 | 571 | 16 | 44 | 36 | 0.51 | |
| 895 | 7.267061772 | down | 0.0000146 | 0.001125207 | CD408455 | 594 | 56 | 144 | 38 | 7e−22 | |
| 896 | 7.31369684 | down | 0.000639204 | 0.007844377 | CD406958 | 601 | 103 | 119 | 86 | 7e−57 | |
| 897 | 7.490615584 | down | 0.00000201 | 0.000707618 | CD416008 | 320 | 7 | 19 | 36 | 4.1 | |
| 898 | 7.613534506 | down | 0.001134447 | 0.011238106 | BQ785103 | 271 | 19 | 67 | 28 | 5.5 | |
| 899 | 7.795957744 | down | 0.000964551 | 0.010114479 | BG509121 | 510 | 73 | 123 | 59 | 2e−33 | aging |
| 900 | 7.822947293 | down | 0.000017 | 0.001184989 | BU544997 | 376 | 37 | 85 | 43 | 5e−11 | |
| 901 | 7.908159019 | down | 0.000563829 | 0.007198868 | CF809113 | 741 | 66 | 143 | 46 | 5e−30 | protein folding |
| 902 | 7.942588667 | down | 0.00070002 | 0.008290479 | CD414525 | 589 | 26 | 91 | 28 | 1e−06 | |
| 903 | 8.351005095 | down | 0.000286276 | 0.004760849 | U36191.1 | | | | | | lipoxygenase |
| 904 | 8.512324712 | down | 0.000021 | 0.001283285 | BI321925 | 256 | 13 | 42 | 30 | 1.1 | |
| 905 | 8.51871477 | down | 0.003625272 | 0.02374128 | CF808220 | 620 | 50 | 114 | 43 | 1e−19 | 40S ribosomal S4 protein |
| 906 | 8.783386049 | down | 0.001216856 | 0.011718521 | BU081272 | 488 | 13 | 27 | 48 | 0.50 | |
| 907 | 8.951869877 | down | 0.00000845 | 0.000900892 | BQ298381 | 389 | 13 | 22 | 59 | 1.9 | |
| 908 | 8.985187033 | down | 0.000361564 | 0.005454491 | U36191.1 | | | | | | lipoxygenase |
| 909 | 9.242782138 | down | 0.00000647 | 0.000848516 | AW394497 | 258 | 12 | 23 | 52 | 9.4 | |
| 910 | 9.247318621 | down | 0.0000205 | 0.001272227 | BQ785389 | 263 | 14 | 41 | 34 | 4.2 | |
| 911 | 9.393348669 | down | 0.000269438 | 0.00458997 | BQ298295 | 412 | 22 | 90 | 24 | 3.8 | |
| 912 | 9.495972212 | down | 0.0000991 | 0.002636034 | BI945212 | 440 | 18 | 34 | 52 | 3e−06 | proteolysis and peptidolysis |
| 913 | 9.516647114 | down | 0.00000595 | 0.000830526 | CD396692 | 603 | 22 | 30 | 73 | 3e−07 | photosynthesis light harvesting |
| 914 | 9.53589734 | down | 0.001037457 | 0.010664167 | AW757140 | 416 | 15 | 66 | 22 | 3.9 | |
| 915 | 10.08216336 | down | 0.001459164 | 0.013083526 | BQ272624 | 466 | 54 | 97 | 55 | 2e−24 | cysteine proteinase inhibitor |
| 916 | 10.88312873 | down | 0.0000255 | 0.001374317 | AI930800 | 451 | 121 | 141 | 85 | 1e−66 | carbohydrate metabolism |
| 917 | 10.91865906 | down | 0.000607228 | 0.007590376 | M64267.1 | | | | | | Fe-superoxide dismutase |
| 918 | 10.98194446 | down | 0.0000636 | 0.002125098 | BQ628525 | 450 | 71 | 95 | 74 | 9e−39 | response to pathogenic bacteria |
| | | | | | | | | | | | (incompatible interaction) |
| 919 | 11.27757119 | down | 0.0000471 | 0.001823768 | AW507983 | 412 | 38 | 48 | 79 | 2e−17 | |
| 920 | 12.32910756 | down | 0.002383998 | 0.018049858 | BU765327 | 570 | 20 | 27 | 74 | 4e−06 | 40S ribosomal S4 protein |
| 921 | 12.53165257 | down | 0.00003 | 0.00147742 | BQ628525 | 450 | 71 | 95 | 74 | 9e−39 | response to pathogenic bacteria |
| | | | | | | | | | | | (incompatible interaction) |
| 922 | 12.58118327 | down | 0.00000256 | 0.000712455 | CF806342 | 671 | 100 | 221 | 45 | 1e−56 | carbohydrate metabolism |
| 923 | 12.5983927 | down | 0.000960499 | 0.010091113 | BG511448 | 514 | 91 | 154 | 59 | 2e−47 | response to ethylene stimulus |
| 924 | 12.97664974 | down | 0.0000202 | 0.001260874 | BE807213 | 252 | 18 | 50 | 36 | 3.3 | regulation of transcription\, DNA- |
| | | | | | | | | | | | dependent |
| 925 | 13.43229811 | down | 0.000240771 | 0.004304359 | CA936258 | 478 | 10 | 19 | 52 | 0.21 | lipid transport |
| 926 | 13.78857747 | down | 0.0000212 | 0.001286626 | BI945352 | | | | | | |
| 927 | 13.81701359 | down | 0.000147184 | 0.003224335 | BG508637 | 441 | 33 | 43 | 76 | 9e−14 | |
| 928 | 14.59039197 | down | 0.000832553 | 0.009242976 | BF324935 | 663 | 16 | 26 | 61 | 6e−04 | proteolysis and peptidolysis |
| 929 | 14.8748104 | down | 0.000624124 | 0.007737606 | BF324935 | 663 | 16 | 26 | 61 | 6e−04 | vacuolar processing enzyme 2, |
| | | | | | | | | | | | proteolysis and peptidolysis |
| 930 | 15.90088465 | down | 0.00000182 | 0.000704945 | CD400517 | 579 | 31 | 82 | 37 | 7e−06 | |
| 931 | 16.57873599 | down | 0.00000735 | 0.000884012 | CD406819 | 479 | 11 | 28 | 39 | 0.48 | G-protein coupled receptor protein |
| | | | | | | | | | | | signaling pathway |
| 932 | 17.04813447 | down | 0.00000174 | 0.000704945 | BM520414 | 367 | 12 | 25 | 48 | 0.95 | |
| 933 | 17.9752754 | down | 0.0000651 | 0.002148386 | AF108084.1 | | | | | | iron-superoxide dismutase |
| 934 | 27.17339042 | down | 0.000000265 | 0.00067462 | U63725.1 | | | | | | metalloproteinase |
| 935 | 27.76198587 | down | 0.00000214 | 0.000707618 | CD409405 | 377 | 11 | 20 | 55 | 1.3 | |
| 936 | 47.38766879 | down | 0.0000032 | 0.000723963 | CD397232 | 594 | 64 | 138 | 46 | 5e−25 | Polyubiquitin, partial |
| 937 | 2.000135769 | up | 0.0000619 | 0.002095846 | CD414265 | 618 | 33 | 81 | 40 | 1e−10 | |
| 938 | 2.000199918 | up | 0.000363823 | 0.00547067 | BE820500 | 625 | 84 | 126 | 66 | 3e−42 | electron transport |
| 939 | 2.001545838 | up | 0.006790098 | 0.036362578 | AI460913 | 435 | 94 | 99 | 94 | 2e−52 | |
| 940 | 2.001817029 | up | 0.000211247 | 0.003990314 | BU080862 | 574 | 155 | 191 | 81 | 2e−84 | |
| 941 | 2.00244534 | up | 0.003193549 | 0.021855919 | BM731619 | 545 | 144 | 151 | 95 | 1e−79 | |
| 942 | 2.002733963 | up | 0.00015794 | 0.003347889 | BU964742 | 444 | 43 | 79 | 54 | 9e−19 | transport |
| 943 | 2.002817703 | up | 0.001257953 | 0.011954684 | CD405904 | 711 | 98 | 151 | 64 | 5e−48 | regulation of transcription |
| 944 | 2.0030488 | up | 0.000158511 | 0.003355218 | BU549035 | 396 | 13 | 24 | 54 | 0.23 | protein biosynthesis |
| 945 | 2.003074738 | up | 0.009817029 | 0.04645393 | BI321141 | 571 | 41 | 64 | 64 | 7e−16 | protein biosynthesis |
| 946 | 2.003135224 | up | 0.0000935 | 0.002553736 | BU548296 | 543 | 39 | 119 | 32 | 6e−09 | |
| 947 | 2.003387015 | up | 0.009928821 | 0.046793969 | AI460581 | 426 | 50 | 62 | 80 | 1e−26 | proteolysis and peptidolysis |
| 948 | 2.003575201 | up | 0.005239604 | 0.030512846 | CD396813 | 686 | 127 | 149 | 85 | 2e−69 | |
| 949 | 2.003683714 | up | 0.000340199 | 0.005277719 | BM526500 | 493 | 60 | 90 | 66 | 7e−32 | |
| 950 | 2.004355423 | up | 0.000669707 | 0.008042894 | BG406519 | 464 | 21 | 33 | 63 | 9e−05 | |
| 951 | 2.004863837 | up | 0.000911371 | 0.009758134 | BQ298636 | 434 | 14 | 23 | 60 | 0.17 | |
| 952 | 2.005130499 | up | 0.001468574 | 0.013142743 | AW351283 | 678 | 106 | 140 | 75 | 2e−56 | |
| 953 | 2.005177726 | up | 0.000743473 | 0.008641838 | BG047325 | 381 | 44 | 60 | 73 | 7e−19 | |
| 954 | 2.005423578 | up | 0.0000666 | 0.002171397 | AW306916 | 428 | 70 | 144 | 48 | 2e−32 | |
| 955 | 2.005427631 | up | 0.000977885 | 0.01020593 | BU544119 | 624 | 61 | 105 | 58 | 4e−28 | two-component signal transduction |
| | | | | | | | | | | | system (phosphorelay) |
| 956 | 2.005431112 | up | 0.000242089 | 0.004322552 | BI321390 | 582 | 26 | 106 | 24 | 0.70 | |
| 957 | 2.005680809 | up | 0.00159179 | 0.013860812 | AW310171 | 465 | 32 | 44 | 72 | 1e−12 | nucleosome assembly |
| 958 | 2.005752766 | up | 0.000687476 | 0.008201312 | CD407681 | 443 | 31 | 42 | 73 | 1e−12 | |
| 959 | 2.006409455 | up | 0.000202217 | 0.003874772 | BE607581 | 484 | 80 | 156 | 51 | 3e−40 | |
| 960 | 2.006491563 | up | 0.003667341 | 0.023909466 | CF807112 | 391 | 109 | 115 | 94 | 7e−59 | translational initiation |
| 961 | 2.006581901 | up | 0.000181735 | 0.003658009 | BM085822 | 559 | 130 | 152 | 85 | 7e−75 | |
| 962 | 2.006593513 | up | 0.001886846 | 0.015472385 | BQ453430 | 564 | 134 | 165 | 81 | 2e−78 | |
| 963 | 2.006692784 | up | 0.000702084 | 0.008307068 | BM309527 | 519 | 12 | 32 | 37 | 0.95 | lipid metabolism |
| 964 | 2.006968995 | up | 0.0000544 | 0.001979942 | BU764183 | 625 | 56 | 104 | 53 | 4e−18 | protein folding |
| 965 | 2.007200206 | up | 0.000102626 | 0.002675093 | BU548422 | 316 | 26 | 65 | 40 | 1e−04 | |
| 966 | 2.008014508 | up | 0.000649584 | 0.007914955 | CD395860 | 480 | 42 | 54 | 77 | 7e−21 | |
| 967 | 2.008123018 | up | 0.000743935 | 0.008641871 | AW310724 | 430 | 46 | 80 | 57 | 1e−18 | |
| 968 | 2.008299326 | up | 0.0000492 | 0.001873618 | CA784534 | 735 | 82 | 173 | 47 | 5e−40 | intracellular protein transport |
| 969 | 2.008915742 | up | 0.0000874 | 0.002487774 | BQ629982 | 456 | 22 | 37 | 59 | 2e−07 | protein modification |
| 970 | 2.009408493 | up | 0.000284388 | 0.004739979 | AW348568 | 687 | 116 | 152 | 76 | 1e−60 | metabolism |
| 971 | 2.009712221 | up | 0.000105631 | 0.00271209 | CD404769 | 467 | 27 | 35 | 77 | 6e−09 | metabolism |
| 972 | 2.009923082 | up | 0.000701297 | 0.008300368 | BG653978 | 358 | 10 | 26 | 38 | 1.1 | protein amino acid phosphorylation |
| 973 | 2.010031739 | up | 0.000747337 | 0.008665329 | BU549065 | 690 | 55 | 171 | 32 | 2e−19 | copper ion transport |
| 974 | 2.010183281 | up | 0.001410096 | 0.012821776 | BM094841 | 565 | 95 | 159 | 59 | 3e−50 | regulation of cell cycle |
| 975 | 2.010210723 | up | 0.000459506 | 0.006344924 | BM309439 | 529 | 53 | 115 | 46 | 8e−22 | |
| 976 | 2.010750114 | up | 0.001615559 | 0.013985643 | BM093848 | 229 | 7 | 17 | 41 | 3.2 | |
| 977 | 2.010816712 | up | 0.010283746 | 0.047926256 | BG652339 | 482 | 116 | 160 | 72 | 9e−67 | glycolysis |
| 978 | 2.010931946 | up | 0.0000912 | 0.002541909 | BU082425 | 619 | 103 | 192 | 53 | 1e−51 | |
| 979 | 2.010989733 | up | 0.000541225 | 0.007008223 | BG352513 | 539 | 35 | 79 | 44 | 9e−16 | |
| 980 | 2.01170346 | up | 0.000285625 | 0.004755864 | BM092431 | 418 | 33 | 65 | 50 | 3e−11 | regulation of transcription |
| 981 | 2.011995509 | up | 0.005426083 | 0.031245666 | BE657393 | 798 | 162 | 214 | 75 | 3e−94 | |
| 982 | 2.012575361 | up | 0.000633205 | 0.007811531 | AW350048 | 819 | 116 | 177 | 65 | 4e−56 | ribosomal protein S6 |
| 983 | 2.012597647 | up | 0.0000881 | 0.002497786 | BU965088 | 633 | 129 | 165 | 78 | 5e−73 | electron transport |
| 984 | 2.012995308 | up | 0.001609856 | 0.013949118 | BQ298655 | 396 | 13 | 26 | 50 | 5.7 | cell growth and/or maintenance |
| 985 | 2.013546995 | up | 0.001523105 | 0.013470507 | BU549354 | 562 | 96 | 128 | 75 | 2e−51 | actin cytoskeleton organization and |
| | | | | | | | | | | | biogenesis |
| 986 | 2.01385177 | up | 0.004901676 | 0.029177344 | CD417415 | 532 | 75 | 97 | 77 | 4e−39 | response to wounding |
| 987 | 2.014211544 | up | 0.0003406 | 0.005277719 | AW397102 | 341 | 43 | 93 | 46 | 2e−19 | |
| 988 | 2.014288987 | up | 0.002020479 | 0.016252844 | CD398987 | 710 | 118 | 138 | 85 | 4e−74 | |
| 989 | 2.015059083 | up | 0.000409387 | 0.005868406 | AW734461 | 453 | 57 | 82 | 69 | 4e−25 | carbohydrate metabolism |
| 990 | 2.015102452 | up | 0.000425206 | 0.006023998 | BU578399 | 408 | 78 | 136 | 57 | 1e−37 | phenylalanyl-tRNA aminoacylation |
| 991 | 2.015385931 | up | 0.0000527 | 0.001953259 | CF808750 | 596 | 71 | 129 | 55 | 3e−29 | G-protein coupled receptor protein |
| | | | | | | | | | | | signaling pathway |
| 992 | 2.015389255 | up | 0.001444357 | 0.013003366 | BG509166 | 362 | 21 | 29 | 72 | 1e−05 | |
| 993 | 2.016132066 | up | 0.000021 | 0.001283285 | BU551348 | 631 | 107 | 145 | 73 | 2e−58 | transport |
| 994 | 2.016238686 | up | 0.005513002 | 0.031572176 | CD391504 | 683 | 20 | 77 | 25 | 0.54 | |
| 995 | 2.016755536 | up | 0.0000149 | 0.001128257 | BU548183 | 542 | 39 | 121 | 32 | 0.007 | transport |
| 996 | 2.016829984 | up | 0.000833278 | 0.009242976 | CD415458 | 628 | 86 | 120 | 71 | 3e−45 | |
| 997 | 2.017010161 | up | 0.000975344 | 0.010190717 | BE608631 | 451 | 71 | 74 | 95 | 5e−34 | regulation of phosphorylation |
| 998 | 2.017425032 | up | 0.003281073 | 0.022283482 | BM092174 | 450 | 28 | 67 | 41 | 4e−07 | |
| 999 | 2.01778442 | up | 0.001031298 | 0.010623251 | BM308100 | 332 | 14 | 14 | 100 | 0.005 | establishment and/or maintenance of |
| | | | | | | | | | | | chromatin architecture |
| 1000 | 2.018147992 | up | 0.000755808 | 0.008720536 | CD401186 | 693 | 119 | 151 | 78 | 3e−58 | |
| 1001 | 2.018413565 | up | 0.000153439 | 0.003313124 | BF595972 | 599 | 83 | 112 | 74 | 1e−46 | |
| 1002 | 2.01873332 | up | 0.001467681 | 0.013140999 | CA802485 | 365 | 11 | 25 | 44 | 4.7 | intracellular signaling cascade |
| 1003 | 2.01887418 | up | 0.000443567 | 0.006197742 | BU550149 | 736 | 20 | 57 | 35 | 1.0 | |
| 1004 | 2.019284302 | up | 0.001794897 | 0.015022977 | BG042731 | 480 | 50 | 80 | 62 | 2e−22 | |
| 1005 | 2.01981207 | up | 0.003158801 | 0.021717992 | AW759569 | 618 | 161 | 203 | 79 | 6e−96 | biosynthesis |
| 1006 | 2.019876399 | up | 0.000787406 | 0.008937084 | BG790666 | 511 | 111 | 170 | 65 | 1e−55 | |
| 1007 | 2.020697415 | up | 0.000233749 | 0.004241302 | BM086170 | 591 | 141 | 162 | 87 | 4e−78 | histidine biosynthesis |
| 1008 | 2.021256519 | up | 0.000477264 | 0.006496916 | BG653021 | 393 | 18 | 21 | 85 | 3e−04 | |
| 1009 | 2.02160766 | up | 0.000649886 | 0.007916069 | BU577819 | 408 | 109 | 135 | 80 | 5e−59 | transport |
| 1010 | 2.022980993 | up | 0.00031694 | 0.005043386 | BE658830 | 578 | 10 | 22 | 45 | 10.0 | |
| 1011 | 2.023064149 | up | 0.0000832 | 0.002435687 | BU964767 | 614 | 176 | 206 | 85 | 1e−95 | |
| 1012 | 2.023802618 | up | 0.003648666 | 0.023855365 | BQ079729 | 576 | 74 | 197 | 37 | 1e−27 | metabolism |
| 1013 | 2.024567322 | up | 0.000134835 | 0.003077454 | BI785606 | 558 | 74 | 88 | 84 | 7e−38 | arginine biosynthesis |
| 1014 | 2.024688902 | up | 0.000244115 | 0.004340089 | CD394657 | 287 | 18 | 61 | 29 | 1.8 | aromatic compound metabolism |
| 1015 | 2.02491745 | up | 0.001427171 | 0.012906284 | BE661461 | 905 | 146 | 173 | 84 | 5e−85 | nucleosome assembly |
| 1016 | 2.024999266 | up | 0.008757948 | 0.043061629 | BI944138 | 344 | 31 | 56 | 55 | 3e−13 | |
| 1017 | 2.025593156 | up | 0.000115898 | 0.002848619 | CD414455 | 630 | 19 | 55 | 34 | 5e−04 | protein folding |
| 1018 | 2.025624477 | up | 0.000052 | 0.001943186 | CA800169 | 686 | 67 | 92 | 72 | 8e−34 | |
| 1019 | 2.026170432 | up | 0.002151217 | 0.016977731 | CA782177 | 335 | 12 | 41 | 29 | 9.4 | |
| 1020 | 2.026609658 | up | 0.0000713 | 0.002243384 | CF808744 | 703 | 134 | 198 | 67 | 2e−67 | fatty acid biosynthesis |
| 1021 | 2.027066118 | up | 0.000259595 | 0.004490311 | BI973058 | 595 | 135 | 147 | 91 | 5e−77 | |
| 1022 | 2.027241608 | up | 0.0000864 | 0.002469742 | BI945472 | 470 | 44 | 67 | 65 | 4e−21 | arginine biosynthesis |
| 1023 | 2.027371893 | up | 0.001255218 | 0.011937746 | CF806846 | 617 | 133 | 170 | 78 | 6e−78 | |
| 1024 | 2.027771756 | up | 0.002515354 | 0.018708265 | BI969411 | 770 | 98 | 152 | 64 | 1e−59 | |
| 1025 | 2.02817441 | up | 0.001188249 | 0.011558001 | BE611098 | 408 | 36 | 55 | 65 | 6e−16 | regulation of transcription |
| 1026 | 2.028350826 | up | 0.0000557 | 0.001994018 | BM086384 | 545 | 63 | 100 | 63 | 1e−32 | DNA replication |
| 1027 | 2.028378476 | up | 0.000347948 | 0.005339087 | BG653819 | 279 | 13 | 33 | 39 | 1.9 | |
| 1028 | 2.02869883 | up | 0.004324701 | 0.026776841 | BG511483 | 218 | 14 | 29 | 48 | 4.2 | oligopeptide transport |
| 1029 | 2.028866336 | up | 0.00048684 | 0.006585943 | BI784912 | 455 | 47 | 68 | 69 | 2e−23 | |
| 1030 | 2.029834471 | up | 0.0000567 | 0.002008532 | CA802777 | 427 | 30 | 39 | 76 | 2e−11 | |
| 1031 | 2.030022836 | up | 0.002355022 | 0.017894018 | BU548694 | 621 | 66 | 111 | 59 | 4e−32 | carbohydrate metabolism |
| 1032 | 2.030297642 | up | 0.004491657 | 0.027524356 | BG045664 | 558 | 135 | 151 | 89 | 5e−76 | galactose metabolism |
| 1033 | 2.030441864 | up | 0.0000424 | 0.001755385 | BU550802 | 686 | 16 | 43 | 37 | 2.0 | regulation of transcription |
| 1034 | 2.03047518 | up | 0.000269694 | 0.004592243 | CD400873 | 548 | 19 | 47 | 40 | 2.4 | |
| 1035 | 2.030610679 | up | 0.000475627 | 0.00648603 | BI944899 | 145 | 10 | 18 | 55 | 5.4 | G-protein coupled receptor protein |
| | | | | | | | | | | | signaling pathway |
| 1036 | 2.030953891 | up | 0.005910561 | 0.033147437 | AW507595 | 446 | 42 | 86 | 48 | 1e−18 | |
| 1037 | 2.032157258 | up | 0.000789816 | 0.00894856 | BU761057 | 483 | 128 | 161 | 79 | 1e−71 | |
| 1038 | 2.032479216 | up | 0.000538337 | 0.006982419 | BM308414 | 530 | 67 | 142 | 47 | 3e−29 | protein biosynthesis |
| 1039 | 2.032719504 | up | 0.0000942 | 0.002560554 | CD411452 | 640 | 70 | 125 | 56 | 1e−29 | |
| 1040 | 2.03298234 | up | 0.000492644 | 0.006632463 | AW349138 | 656 | 19 | 58 | 32 | 0.059 | |
| 1041 | 2.033030389 | up | 0.002557662 | 0.01892221 | BM091939 | 521 | 140 | 170 | 82 | 2e−81 | fatty acid biosynthesis |
| 1042 | 2.033126798 | up | 0.000470225 | 0.006433374 | CD392760 | 403 | 11 | 22 | 50 | 1.6 | G-protein coupled receptor protein |
| | | | | | | | | | | | signaling pathway |
| 1043 | 2.03314691 | up | 0.000130604 | 0.00303449 | BF325197 | 427 | 58 | 84 | 69 | 7e−29 | DNA repair |
| 1044 | 2.035760872 | up | 0.000180189 | 0.003643894 | CD390492 | 642 | 78 | 105 | 74 | 2e−43 | |
| 1045 | 2.036429103 | up | 0.002328327 | 0.017749833 | CA784222 | 746 | 119 | 164 | 72 | 2e−63 | |
| 1046 | 2.036624886 | up | 0.00045417 | 0.006308398 | BM528077 | 551 | 106 | 186 | 56 | 8e−55 | |
| 1047 | 2.036733721 | up | 0.000224768 | 0.004134189 | BE611879 | 448 | 80 | 133 | 60 | 1e−41 | |
| 1048 | 2.037525043 | up | 0.007068039 | 0.037318529 | CD396938 | 584 | 87 | 120 | 72 | 9e−49 | electron transport |
| 1049 | 2.03907602 | up | 0.001528242 | 0.013486401 | BE659002 | 511 | 99 | 100 | 99 | 1e−51 | |
| 1050 | 2.039314923 | up | 0.0000447 | 0.001789988 | AW310166 | 613 | 64 | 95 | 67 | 2e−29 | |
| 1051 | 2.039315284 | up | 0.0000222 | 0.001314393 | BI471792 | 525 | 62 | 88 | 70 | 3e−36 | |
| 1052 | 2.039389689 | up | 0.000810986 | 0.009084828 | CD486940 | 579 | 26 | 30 | 86 | 1e−08 | ciliary/flagellar motility |
| 1053 | 2.03941606 | up | 0.000459833 | 0.006344924 | CD403533 | 428 | 26 | 97 | 26 | 0.83 | |
| 1054 | 2.039494356 | up | 0.000119343 | 0.002897718 | AW761338 | 465 | 115 | 153 | 75 | 6e−64 | |
| 1055 | 2.039914711 | up | 0.002518698 | 0.018719284 | BE610326 | 279 | 11 | 38 | 28 | 0.84 | translational elongation |
| 1056 | 2.040080537 | up | 0.000367357 | 0.005490568 | BM143204 | 464 | 109 | 188 | 57 | 3e−51 | potassium ion transport |
| 1057 | 2.040229149 | up | 0.0000568 | 0.002008532 | BE022823 | 514 | 142 | 169 | 84 | 1e−83 | |
| 1058 | 2.041387465 | up | 0.000640914 | 0.007855112 | BQ610137 | 173 | 11 | 25 | 44 | 1.5 | |
| 1059 | 2.041488358 | up | 0.000224887 | 0.004134366 | BE803335 | 468 | 27 | 30 | 90 | 9e−10 | |
| 1060 | 2.041541567 | up | 0.002876585 | 0.020469135 | BU550174 | 712 | 95 | 161 | 59 | 8e−45 | |
| 1061 | 2.04239887 | up | 0.0000851 | 0.002462679 | AW309912 | 676 | 27 | 66 | 40 | 6e−07 | |
| 1062 | 2.042538033 | up | 0.0000205 | 0.001269383 | AW781752 | 524 | 70 | 151 | 46 | 7e−25 | |
| 1063 | 2.042630087 | up | 0.000784665 | 0.008924824 | BQ628751 | 420 | 28 | 53 | 52 | 2e−09 | |
| 1064 | 2.042874752 | up | 0.006417076 | 0.035045263 | BE058294 | 258 | 10 | 20 | 50 | 4.2 | |
| 1065 | 2.042989748 | up | 0.001204928 | 0.011646321 | CD395372 | | | | | | |
| 1066 | 2.043667532 | up | 0.000280331 | 0.0047073 | BI317540 | 519 | 27 | 39 | 69 | 6e−09 | |
| 1067 | 2.043758916 | up | 0.002711748 | 0.019668275 | BE659804 | 529 | 86 | 111 | 77 | 7e−42 | transport |
| 1068 | 2.0438334 | up | 0.0000887 | 0.002506437 | BM091829 | 490 | 21 | 90 | 23 | 0.22 | |
| 1069 | 2.044429891 | up | 0.000952768 | 0.010030059 | BM885282 | 563 | 118 | 188 | 62 | 7e−67 | |
| 1070 | 2.04453014 | up | 0.0000813 | 0.002410039 | BG652296 | 340 | 15 | 38 | 39 | 3.2 | |
| 1071 | 2.044859517 | up | 0.000377014 | 0.005587476 | CA820600 | 586 | 80 | 102 | 78 | 5e−42 | |
| 1072 | 2.045223399 | up | 0.000122813 | 0.002947703 | BU579208 | 589 | 54 | 78 | 69 | 5e−26 | |
| 1073 | 2.045533299 | up | 0.001608235 | 0.013939648 | BU544455 | 597 | 13 | 30 | 43 | 4.7 | electron transport |
| 1074 | 2.045697542 | up | 0.0000311 | 0.001503193 | AW733220 | | | | | | |
| 1075 | 2.046628714 | up | 0.002705048 | 0.019642409 | BE805444 | 385 | 55 | 91 | 60 | 2e−27 | regulation of transcription\, DNA- |
| | | | | | | | | | | | dependent |
| 1076 | 2.047072927 | up | 0.000292565 | 0.004809299 | BU577464 | 488 | 91 | 137 | 66 | 8e−50 | chloride transport |
| 1077 | 2.04778021 | up | 0.001393725 | 0.012742874 | BE346592 | 621 | 133 | 193 | 68 | 7e−69 | DNA replication initiation |
| 1078 | 2.048120712 | up | 0.000120843 | 0.00291558 | AW830902 | 521 | 60 | 80 | 75 | 1e−31 | |
| 1079 | 2.048194324 | up | 0.000038 | 0.001660614 | BU761927 | 591 | 81 | 120 | 67 | 1e−40 | protein biosynthesis |
| 1080 | 2.048478716 | up | 0.00027951 | 0.004699809 | AI900305 | 622 | 83 | 169 | 49 | 2e−39 | RNA processing |
| 1081 | 2.049492971 | up | 0.0000557 | 0.001994018 | BG882263 | 297 | 32 | 78 | 41 | 1e−09 | electron transport |
| 1082 | 2.049503726 | up | 0.007276392 | 0.038023647 | AW317898 | 607 | 117 | 155 | 75 | 4e−64 | transport |
| 1083 | 2.050403869 | up | 0.001250844 | 0.011905176 | AW309249 | 618 | 39 | 63 | 61 | 6e−17 | |
| 1084 | 2.051205889 | up | 0.001675733 | 0.014331712 | BF324399 | 651 | 138 | 216 | 63 | 4e−71 | response to heat |
| 1085 | 2.051408392 | up | 0.0000454 | 0.001798383 | CD402933 | 434 | 30 | 57 | 52 | 3e−11 | G-protein coupled receptor protein |
| | | | | | | | | | | | signaling pathway |
| 1086 | 2.051901291 | up | 0.0000852 | 0.002462679 | BE821323 | 451 | 25 | 37 | 67 | 1e−08 | |
| 1087 | 2.051971154 | up | 0.000887484 | 0.009603513 | BG550828 | 390 | 9 | 26 | 34 | 9.6 | |
| 1088 | 2.053148877 | up | 0.001196003 | 0.011608983 | BE823222 | 619 | 53 | 91 | 58 | 2e−19 | |
| 1089 | 2.053252494 | up | 0.00027328 | 0.004630251 | BI316897 | 350 | 18 | 59 | 30 | 0.64 | |
| 1090 | 2.053319574 | up | 0.00000976 | 0.000945362 | BI973736 | 568 | 106 | 190 | 55 | 2e−56 | carbohydrate metabolism |
| 1091 | 2.053839852 | up | 0.000311758 | 0.005009165 | CD399849 | 525 | 88 | 137 | 64 | 3e−49 | |
| 1092 | 2.054131903 | up | 0.000821507 | 0.00914747 | BU926270 | 637 | 11 | 16 | 68 | 2.4 | |
| 1093 | 2.05428281 | up | 0.001501343 | 0.013346844 | BG047185 | 565 | 52 | 93 | 55 | 1e−20 | |
| 1094 | 2.055190664 | up | 0.00171143 | 0.014534594 | BM523130 | 593 | 73 | 89 | 82 | 5e−40 | |
| 1095 | 2.057009875 | up | 0.000194858 | 0.003785091 | BF066739 | 461 | 119 | 153 | 77 | 1e−64 | metabolism |
| 1096 | 2.057199775 | up | 0.00019482 | 0.003785091 | BE211685 | 441 | 15 | 29 | 51 | 5e−04 | translational elongation |
| 1097 | 2.057219778 | up | 0.001775282 | 0.014921893 | CA801701 | 626 | 128 | 185 | 69 | 1e−64 | gluconeogenesis |
| 1098 | 2.05776027 | up | 0.0000344 | 0.001562903 | BQ454033 | 428 | 54 | 80 | 67 | 8e−23 | phospholipid biosynthesis |
| 1099 | 2.057795903 | up | 0.000979898 | 0.010215604 | CD402292 | 478 | 49 | 72 | 68 | 1e−22 | protein biosynthesis |
| 1100 | 2.058384192 | up | 0.002314468 | 0.017694388 | CF806994 | 509 | 86 | 104 | 82 | 2e−45 | metabolism |
| 1101 | 2.058605324 | up | 0.000307772 | 0.004957838 | BU082329 | 495 | 118 | 168 | 70 | 4e−65 | viral replication complex formation |
| | | | | | | | | | | | and maintenance |
| 1102 | 2.058839067 | up | 0.0000925 | 0.002551441 | BI424590 | 556 | 131 | 185 | 70 | 4e−66 | |
| 1103 | 2.058870733 | up | 0.000108771 | 0.002771905 | CA853488 | 487 | 11 | 34 | 32 | 4.2 | gibberellic acid biosynthesis |
| 1104 | 2.059131738 | up | 0.000206997 | 0.003936298 | AW704196 | 603 | 157 | 180 | 87 | 8e−88 | |
| 1105 | 2.059387534 | up | 0.000181763 | 0.003658009 | BI321747 | 470 | 15 | 41 | 36 | 3.9 | |
| 1106 | 2.059774638 | up | 0.000223304 | 0.004115311 | CA802358 | 477 | 55 | 91 | 60 | 3e−28 | |
| 1107 | 2.059952494 | up | 0.000219898 | 0.004076965 | AI494968 | 384 | 51 | 114 | 44 | 8e−17 | transport |
| 1108 | 2.060061526 | up | 0.0000545 | 0.001979942 | BF009043 | 647 | 149 | 214 | 69 | 3e−89 | |
| 1109 | 2.060209555 | up | 0.00868845 | 0.042820668 | BM731941 | 545 | 63 | 109 | 57 | 2e−32 | |
| 1110 | 2.060449232 | up | 0.000111516 | 0.002796365 | BG155079 | 406 | 12 | 22 | 54 | 0.19 | protein amino acid phosphorylation |
| 1111 | 2.060566206 | up | 0.009959419 | 0.046884987 | BE190346 | 394 | 26 | 80 | 32 | 1.2 | ubiquitin-dependent protein |
| | | | | | | | | | | | catabolism |
| 1112 | 2.060946404 | up | 0.0000947 | 0.002566638 | CD410984 | 478 | 66 | 76 | 86 | 5e−32 | histidine biosynthesis |
| 1113 | 2.061367591 | up | 0.0000139 | 0.001105237 | BU081474 | 549 | 85 | 133 | 63 | 4e−48 | |
| 1114 | 2.061867819 | up | 0.001407046 | 0.012808475 | CD411565 | 489 | 34 | 78 | 43 | 4e−10 | |
| 1115 | 2.063004676 | up | 0.000315598 | 0.005038495 | AI440627 | 446 | 109 | 131 | 83 | 6e−61 | protein catabolism |
| 1116 | 2.063371449 | up | 0.000794655 | 0.008969456 | CD401340 | 680 | 120 | 140 | 85 | 2e−65 | protein biosynthesis |
| 1117 | 2.064552903 | up | 0.000488523 | 0.006600045 | BQ612759 | 395 | 13 | 29 | 44 | 0.52 | |
| 1118 | 2.064898177 | up | 0.000257484 | 0.004473449 | BI969133 | 719 | 37 | 54 | 68 | 8e−16 | |
| 1119 | 2.064926772 | up | 0.002479123 | 0.018546085 | BG237370 | 426 | 79 | 101 | 78 | 5e−41 | |
| 1120 | 2.065006086 | up | 0.000449547 | 0.006262193 | BE822429 | 614 | 37 | 75 | 49 | 1e−15 | G-protein coupled receptor protein |
| | | | | | | | | | | | signaling pathway |
| 1121 | 2.065928564 | up | 0.00056903 | 0.007250504 | BF425152 | 415 | 63 | 118 | 53 | 1e−30 | transport |
| 1122 | 2.06607574 | up | 0.002687637 | 0.019572105 | BG156403 | 519 | 64 | 135 | 47 | 7e−33 | proline biosynthesis |
| 1123 | 2.067096531 | up | 0.000984594 | 0.010250351 | BI969665 | 747 | 86 | 130 | 66 | 4e−48 | |
| 1124 | 2.067201695 | up | 0.000379828 | 0.005608036 | BM308191 | 597 | 53 | 87 | 60 | 4e−23 | regulation of transcription\, DNA- |
| | | | | | | | | | | | dependent |
| 1125 | 2.067688848 | up | 0.000287861 | 0.004770666 | BE057781 | 470 | 27 | 64 | 42 | 3e−08 | regulation of transcription |
| 1126 | 2.067816457 | up | 0.000491608 | 0.006627727 | BE021655 | 444 | 46 | 77 | 59 | 5e−18 | |
| 1127 | 2.068726811 | up | 0.002328179 | 0.017749833 | BG315904 | 344 | 24 | 31 | 77 | 1e−08 | metabolism |
| 1128 | 2.068771151 | up | 0.002831207 | 0.020235968 | BI471955 | 454 | 60 | 113 | 53 | 3e−24 | |
| 1129 | 2.069323582 | up | 0.000178795 | 0.003632031 | CD409149 | 620 | 74 | 111 | 66 | 4e−36 | |
| 1130 | 2.069503763 | up | 0.000198832 | 0.003834388 | BU083547 | 413 | 12 | 20 | 60 | 5.0 | carbohydrate biosynthesis |
| 1131 | 2.069612719 | up | 0.000227908 | 0.004165475 | CD411220 | 516 | 21 | 74 | 28 | 3.6 | protein amino acid phosphorylation |
| 1132 | 2.070116324 | up | 0.002930885 | 0.020675605 | AW350627 | 809 | 198 | 209 | 94 | e−107 | ubiquitin-dependent protein |
| | | | | | | | | | | | catabolism |
| 1133 | 2.070133325 | up | 0.002634781 | 0.019326178 | AW201986 | 787 | 91 | 119 | 76 | 5e−46 | |
| 1134 | 2.07072802 | up | 0.0000822 | 0.002420756 | CF805768 | 552 | 99 | 115 | 86 | 3e−56 | electron transport |
| 1135 | 2.070827132 | up | 0.000068 | 0.002184143 | BG725538 | 415 | 30 | 86 | 34 | 0.45 | |
| 1136 | 2.07103307 | up | 0.000258725 | 0.004487419 | BG551392 | 342 | 11 | 22 | 50 | 3.2 | |
| 1137 | 2.071143783 | up | 0.000189851 | 0.003732626 | BM308149 | 602 | 100 | 172 | 58 | 6e−51 | |
| 1138 | 2.07151514 | up | 0.000793649 | 0.008963767 | BI967250 | 761 | 97 | 168 | 57 | 8e−49 | proteolysis and peptidolysis |
| 1139 | 2.072718267 | up | 0.0000801 | 0.00238979 | AW350110 | 675 | 88 | 167 | 52 | 7e−38 | regulation of transcription |
| 1140 | 2.072774725 | up | 0.00066941 | 0.008042187 | BQ452617 | 360 | 46 | 71 | 64 | 1e−23 | |
| 1141 | 2.073021345 | up | 0.000899988 | 0.009683106 | CD392202 | 595 | 43 | 53 | 81 | 7e−17 | |
| 1142 | 2.07325694 | up | 0.000431837 | 0.006083568 | BQ785359 | 574 | 167 | 190 | 87 | 1e−97 | |
| 1143 | 2.073312961 | up | 0.002620489 | 0.019240109 | AW350757 | 760 | 113 | 125 | 90 | 2e−61 | |
| 1144 | 2.073368566 | up | 0.00025264 | 0.004420006 | BU550793 | 700 | 126 | 173 | 72 | 4e−76 | |
| 1145 | 2.074090322 | up | 0.000512437 | 0.0067869 | CD407103 | 645 | 58 | 118 | 49 | 1e−21 | |
| 1146 | 2.075254344 | up | 0.003423669 | 0.022857275 | BI942098 | 656 | 47 | 86 | 54 | 8e−18 | 4-coumarate:Coenzyme A ligase |
| | | | | | | | | | | | isoenzyme 4, phenylpropanoid |
| | | | | | | | | | | | metabolism |
| 1147 | 2.075779354 | up | 0.002964507 | 0.020830745 | BU549808 | 721 | 61 | 105 | 58 | 9e−23 | phenylalanyl-tRNA aminoacylation |
| 1148 | 2.07613623 | up | 0.000574339 | 0.007300835 | CF808412 | 156 | 11 | 13 | 84 | 2.4 | |
| 1149 | 2.076498728 | up | 0.000398528 | 0.005789966 | BQ742944 | 597 | 110 | 152 | 72 | 3e−59 | |
| 1150 | 2.076950367 | up | 0.002707307 | 0.019647433 | CF805624 | 536 | 146 | 174 | 83 | 9e−79 | |
| 1151 | 2.077640667 | up | 0.0000384 | 0.001672187 | CF807829 | 553 | 128 | 132 | 96 | 3e−69 | translational initiation |
| 1152 | 2.078175162 | up | 0.000195916 | 0.003796597 | BI942852 | 479 | 120 | 148 | 81 | 1e−66 | DNA metabolism |
| 1153 | 2.07832742 | up | 0.000411261 | 0.005884695 | CD410080 | 606 | 77 | 100 | 77 | 7e−41 | |
| 1154 | 2.078593903 | up | 0.000458338 | 0.006335829 | AW761577 | 369 | 45 | 127 | 35 | 8e−14 | |
| 1155 | 2.078908398 | up | 0.001418426 | 0.012856283 | BU578779 | 576 | 83 | 148 | 56 | 1e−41 | electron transport |
| 1156 | 2.079001554 | up | 0.001829942 | 0.015184521 | AF169021.1 | | | | | | seed maturation protein PM36 |
| 1157 | 2.079265964 | up | 0.0000829 | 0.002431824 | CD414265 | 618 | 33 | 81 | 40 | 1e−10 | |
| 1158 | 2.080390191 | up | 0.00112912 | 0.011211914 | CD397738 | 576 | 42 | 94 | 44 | 6e−18 | |
| 1159 | 2.080736134 | up | 0.0000427 | 0.001761872 | CA783605 | 430 | 56 | 82 | 68 | 7e−29 | cell cycle |
| 1160 | 2.081179126 | up | 0.000299749 | 0.004876631 | CA802980 | 244 | 11 | 21 | 52 | 1.1 | |
| 1161 | 2.081351302 | up | 0.00018756 | 0.00370587 | CA783364 | 710 | 71 | 179 | 39 | 3e−27 | |
| 1162 | 2.081834679 | up | 0.0000625 | 0.002103502 | BM308341 | 586 | 61 | 84 | 72 | 5e−31 | mitochondrial translocation |
| 1163 | 2.082343015 | up | 0.003607404 | 0.023655664 | CA819221 | 545 | 77 | 181 | 42 | 4e−29 | |
| 1164 | 2.083212303 | up | 0.000790729 | 0.00894856 | BI316349 | 552 | 131 | 184 | 71 | 7e−79 | |
| 1165 | 2.083499185 | up | 0.000190675 | 0.003737135 | AW348465 | 700 | 45 | 72 | 62 | 4e−15 | DNA replication initiation |
| 1166 | 2.084020974 | up | 0.000535193 | 0.006960839 | CD395753 | 364 | 23 | 36 | 63 | 9e−09 | |
| 1167 | 2.086036008 | up | 0.001805485 | 0.015074698 | CF806176 | | | | | | |
| 1168 | 2.086084489 | up | 0.0000906 | 0.002529045 | BE611665 | 369 | 42 | 44 | 95 | 4e−20 | nucleocytoplasmic transport |
| 1169 | 2.086140544 | up | 0.000136312 | 0.003092431 | AW351097 | 566 | 47 | 102 | 46 | 5e−14 | |
| 1170 | 2.087001853 | up | 0.000161531 | 0.003401923 | BU090015 | 555 | 71 | 101 | 70 | 4e−36 | |
| 1171 | 2.087182754 | up | 0.000108157 | 0.002761847 | BG551001 | 507 | 132 | 168 | 78 | 3e−71 | protein biosynthesis |
| 1172 | 2.08721268 | up | 0.000216613 | 0.004053564 | BU548617 | 559 | 56 | 88 | 63 | 6e−28 | |
| 1173 | 2.087571421 | up | 0.00167611 | 0.014331712 | CK606640 | 488 | 48 | 55 | 87 | 6e−20 | isoprenoid biosynthesis |
| 1174 | 2.087765784 | up | 0.0000675 | 0.002177084 | BE819998 | 827 | 72 | 160 | 45 | 1e−24 | |
| 1175 | 2.087834647 | up | 0.0000255 | 0.001374317 | CF805757 | 543 | 120 | 167 | 71 | 5e−65 | |
| 1176 | 2.088116553 | up | 0.000311996 | 0.00501086 | AW598515 | 402 | 12 | 30 | 40 | 0.93 | |
| 1177 | 2.088724783 | up | 0.0000101 | 0.000954671 | BI943803 | 446 | 61 | 94 | 64 | 1e−29 | |
| 1178 | 2.090072966 | up | 0.0000333 | 0.001543897 | BI971415 | 689 | 106 | 212 | 50 | 3e−60 | electron transport |
| 1179 | 2.09011082 | up | 0.000354881 | 0.005395505 | AW156714 | 393 | 42 | 58 | 72 | 2e−21 | |
| 1180 | 2.090604582 | up | 0.001374015 | 0.012639328 | BM955045 | 566 | 138 | 170 | 81 | 2e−80 | transport |
| 1181 | 2.090936457 | up | 0.00512358 | 0.030046514 | AI442808 | 547 | 109 | 140 | 77 | 2e−62 | |
| 1182 | 2.091038215 | up | 0.000348024 | 0.005339087 | CD390952 | 439 | 16 | 35 | 45 | 1.1 | DNA replication |
| 1183 | 2.09105356 | up | 0.000146707 | 0.003216456 | BE820518 | 475 | 14 | 40 | 35 | 5.1 | DNA transposition |
| 1184 | 2.092015382 | up | 0.001141852 | 0.011292661 | AW100064 | 455 | 109 | 138 | 78 | 2e−62 | posttranscriptional gene silencing |
| 1185 | 2.092288841 | up | 0.002648717 | 0.019380331 | AI442425 | 541 | 113 | 181 | 62 | 1e−62 | |
| 1186 | 2.092389457 | up | 0.002714813 | 0.019682918 | BE440491 | 462 | 43 | 64 | 67 | 2e−18 | fatty acid biosynthesis |
| 1187 | 2.092926094 | up | 0.000138593 | 0.003129806 | CD407942 | 628 | 126 | 157 | 80 | 3e−68 | protein amino acid phosphorylation |
| 1188 | 2.093073845 | up | 0.0000222 | 0.001314393 | CD399838 | 658 | 39 | 92 | 42 | 4e−16 | electron transport |
| 1189 | 2.093379654 | up | 0.000111841 | 0.002800796 | BE475334 | 430 | 125 | 142 | 88 | 3e−71 | |
| 1190 | 2.093784148 | up | 0.000866557 | 0.009450471 | BM527523 | 569 | 24 | 42 | 57 | 7e−06 | |
| 1191 | 2.094249537 | up | 0.000467265 | 0.006409816 | AI966260 | 525 | 19 | 65 | 29 | 0.75 | electron transport |
| 1192 | 2.094983833 | up | 0.002293389 | 0.017586856 | CD398452 | 508 | 84 | 90 | 93 | 2e−44 | |
| 1193 | 2.094986797 | up | 0.000487066 | 0.006585943 | CD401978 | 674 | 147 | 156 | 94 | 1e−82 | |
| 1194 | 2.097048558 | up | 0.002777665 | 0.019969638 | CF806118 | 657 | 161 | 197 | 81 | 1e−89 | |
| 1195 | 2.097072085 | up | 0.003070802 | 0.02130698 | BG047223 | 478 | 29 | 60 | 48 | 8e−06 | intracellular protein transport |
| 1196 | 2.097517338 | up | 0.000248002 | 0.004383617 | BE821913 | 717 | 119 | 190 | 62 | 7e−58 | DNA metabolism |
| 1197 | 2.098569956 | up | 0.000155021 | 0.003324117 | BM732455 | 568 | 102 | 119 | 85 | 7e−56 | protein-nucleus import |
| 1198 | 2.098901303 | up | 0.000219289 | 0.004075274 | BM177355 | 371 | 20 | 25 | 80 | 8e−08 | |
| 1199 | 2.098979793 | up | 0.006528067 | 0.035422205 | CD417406 | 347 | 24 | 36 | 66 | 4e−10 | transport |
| 1200 | 2.099074754 | up | 0.006118242 | 0.033902702 | CF807384 | 321 | 71 | 81 | 87 | 7e−40 | |
| 1201 | 2.099106585 | up | 0.00034096 | 0.005277719 | BQ453267 | 235 | 12 | 28 | 42 | 1.1 | |
| 1202 | 2.099234551 | up | 0.000271178 | 0.004607433 | BI969586 | 463 | 14 | 26 | 53 | 0.75 | protein biosynthesis |
| 1203 | 2.099730261 | up | 0.0000953 | 0.002573271 | BM177049 | 520 | 19 | 57 | 33 | 0.010 | |
| 1204 | 2.099987781 | up | 0.002888989 | 0.020499235 | AF488307.1 | | | | | | S-adenosylmethionine |
| | | | | | | | | | | | decarboxylase |
| 1205 | 2.100732704 | up | 0.000294204 | 0.004824317 | BE210812 | 471 | 85 | 151 | 56 | 1e−46 | |
| 1206 | 2.101052843 | up | 0.0000905 | 0.002529045 | CD403462 | 423 | 19 | 47 | 40 | 0.21 | |
| 1207 | 2.101230478 | up | 0.001445462 | 0.01300662 | BU579127 | 593 | 115 | 200 | 57 | 2e−58 | mRNA splicing |
| 1208 | 2.10174047 | up | 0.0021716 | 0.017091977 | BE801533 | 147 | 15 | 27 | 55 | 0.044 | response to hypoxia |
| 1209 | 2.101832494 | up | 0.001079296 | 0.010918949 | BQ298655 | 396 | 13 | 26 | 50 | 5.7 | cell growth and/or maintenance |
| 1210 | 2.102476496 | up | 0.000540906 | 0.007008223 | BF071679 | 386 | 95 | 127 | 74 | 1e−50 | RNA processing |
| 1211 | 2.102722884 | up | 0.001324282 | 0.012352906 | BQ297685 | 607 | 20 | 46 | 43 | 0.001 | |
| 1212 | 2.103489534 | up | 0.0000715 | 0.002247932 | CK606265 | 775 | 129 | 188 | 68 | 4e−73 | phosphoenolpyruvate-dependent |
| | | | | | | | | | | | sugar phosphotransferase system |
| 1213 | 2.104180292 | up | 0.000576047 | 0.007315136 | BU545911 | 508 | 14 | 26 | 53 | 5.9 | proteolysis and peptidolysis |
| 1214 | 2.104275905 | up | 0.000281195 | 0.004713407 | BU544017 | 594 | 26 | 86 | 30 | 0.55 | DNA metabolism |
| 1215 | 2.104390833 | up | 0.000120033 | 0.002902004 | CA852398 | 366 | 9 | 19 | 47 | 2.8 | regulation of gene expression\, |
| | | | | | | | | | | | epigenetic |
| 1216 | 2.104490263 | up | 0.0000251 | 0.001368772 | BU761689 | 613 | 126 | 196 | 64 | 8e−67 | |
| 1217 | 2.105075163 | up | 0.000107834 | 0.002756064 | CD402168 | 590 | 64 | 125 | 51 | 1e−34 | |
| 1218 | 2.105869771 | up | 0.000850462 | 0.009364538 | BM307193 | 384 | 24 | 39 | 61 | 3e−08 | Cv Prize protein kinase mRNA., |
| | | | | | | | | | | | cytokinesis |
| 1219 | 2.10637375 | up | 0.000129851 | 0.003028023 | CD394996 | 493 | 19 | 54 | 35 | 1.9 | oligopeptide transport |
| 1220 | 2.106404 | up | 0.000761995 | 0.008770408 | BU760808 | 242 | 18 | 54 | 33 | 0.83 | electron transport |
| 1221 | 2.107305599 | up | 0.005493062 | 0.031501146 | BQ299549 | 563 | 36 | 56 | 64 | 2e−15 | |
| 1222 | 2.108107678 | up | 0.000111612 | 0.002796924 | CD396058 | 314 | 18 | 61 | 29 | 3.2 | protein amino acid phosphorylation |
| 1223 | 2.108171603 | up | 0.000113019 | 0.002817883 | BI427098 | 537 | 45 | 125 | 36 | 4e−11 | |
| 1224 | 2.109711739 | up | 0.000287858 | 0.004770666 | BE806979 | 465 | 47 | 63 | 74 | 4e−25 | electron transport |
| 1225 | 2.109761797 | up | 0.000154435 | 0.003322585 | BM892410 | 455 | 72 | 95 | 75 | 1e−41 | |
| 1226 | 2.109898923 | up | 0.0000281 | 0.001444531 | AW734464 | 456 | 99 | 150 | 66 | 2e−55 | |
| 1227 | 2.110186198 | up | 0.0000431 | 0.00176847 | BE659244 | 713 | 70 | 103 | 67 | 3e−32 | DNA metabolism |
| 1228 | 2.111264642 | up | 0.0000596 | 0.002059649 | BM885268 | 567 | 31 | 72 | 43 | 2e−07 | regulation of transcription |
| 1229 | 2.111369738 | up | 0.000144094 | 0.003182607 | BE805930 | 635 | 158 | 212 | 74 | 4e−82 | purine nucleotide biosynthesis |
| 1230 | 2.111544882 | up | 0.0000156 | 0.001135545 | BE440517 | 461 | 16 | 32 | 50 | 0.99 | metabolism |
| 1231 | 2.11278075 | up | 0.000912959 | 0.009766789 | BE659438 | 256 | 19 | 24 | 79 | 3e−05 | protein biosynthesis |
| 1232 | 2.112896419 | up | 0.0000471 | 0.001823768 | CA800777 | 498 | 91 | 164 | 55 | 3e−50 | transport |
| 1233 | 2.113006073 | up | 0.000503087 | 0.006698452 | CA935724 | 558 | 81 | 116 | 69 | 2e−39 | N-terminal protein myristoylation |
| 1234 | 2.113049913 | up | 0.000429724 | 0.00606515 | CA782572 | 568 | 140 | 155 | 90 | 8e−74 | secretory pathway |
| 1235 | 2.113940827 | up | 0.00021306 | 0.004009017 | BE820822 | 738 | 81 | 137 | 59 | 2e−41 | |
| 1236 | 2.114123376 | up | 0.000246531 | 0.004369942 | BU760770 | | | | | | |
| 1237 | 2.114412549 | up | 0.000406478 | 0.005858599 | CF807780 | 637 | 72 | 131 | 54 | 7e−34 | |
| 1238 | 2.114703066 | up | 0.000780808 | 0.008897094 | BI320306 | 477 | 11 | 23 | 47 | 1.4 | |
| 1239 | 2.114768163 | up | 0.000150406 | 0.003269358 | CD396651 | 671 | 65 | 104 | 62 | 7e−30 | |
| 1240 | 2.115664515 | up | 0.000552802 | 0.007106409 | BQ473314 | 267 | 10 | 30 | 33 | 7.2 | regulation of circadian rhythm |
| 1241 | 2.117072043 | up | 0.001727964 | 0.014638331 | BQ740835 | 358 | 10 | 13 | 76 | 0.86 | carbohydrate metabolism |
| 1242 | 2.117635988 | up | 0.0000436 | 0.001771311 | BM307950 | 548 | 48 | 82 | 58 | 1e−26 | |
| 1243 | 2.117665547 | up | 0.0000257 | 0.001377579 | AW350647 | 656 | 11 | 21 | 52 | 5.5 | electron transport |
| 1244 | 2.117765673 | up | 0.000180961 | 0.003655569 | CA937173 | 512 | 34 | 40 | 85 | 3e−14 | branched chain family amino acid |
| | | | | | | | | | | | biosynthesis |
| 1245 | 2.117898631 | up | 0.0000617 | 0.00209261 | AW471981 | 366 | 92 | 121 | 76 | 3e−51 | metabolism |
| 1246 | 2.118818155 | up | 0.00025888 | 0.004487419 | BG406414 | 349 | 15 | 50 | 30 | 0.13 | |
| 1247 | 2.119136292 | up | 0.0000161 | 0.0011509 | CA935112 | 449 | 57 | 99 | 57 | 2e−28 | |
| 1248 | 2.119158029 | up | 0.000443232 | 0.006195932 | CD400392 | 715 | 139 | 153 | 90 | 8e−80 | |
| 1249 | 2.119305086 | up | 0.003448745 | 0.022939182 | CF807864 | 660 | 145 | 219 | 66 | 1e−82 | proteolysis and peptidolysis |
| 1250 | 2.119595336 | up | 0.000123612 | 0.002950604 | BU764205 | 607 | 66 | 101 | 65 | 2e−31 | |
| 1251 | 2.119782854 | up | 0.000935352 | 0.009927297 | CA820191 | 310 | 10 | 21 | 47 | 9.3 | protein amino acid phosphorylation |
| 1252 | 2.120361875 | up | 0.0000191 | 0.001231955 | BI969629 | 778 | 14 | 38 | 36 | 0.17 | |
| 1253 | 2.120412762 | up | 0.001389111 | 0.01271599 | CD404227 | 636 | 77 | 93 | 82 | 4e−39 | |
| 1254 | 2.120541423 | up | 0.000203005 | 0.003885925 | AW317147 | 460 | 38 | 57 | 66 | 4e−17 | protein biosynthesis |
| 1255 | 2.12089382 | up | 0.000431494 | 0.006081004 | BG352641 | 513 | 23 | 62 | 37 | 6e−06 | |
| 1256 | 2.121157213 | up | 0.005671383 | 0.032190039 | CD403235 | 335 | 12 | 15 | 80 | 0.004 | |
| 1257 | 2.121608531 | up | 0.000185891 | 0.003694813 | BI970809 | 777 | 75 | 129 | 58 | 2e−40 | |
| 1258 | 2.12163047 | up | 0.000179823 | 0.003640408 | BU764067 | 485 | 78 | 109 | 71 | 1e−38 | cation transport |
| 1259 | 2.122136632 | up | 0.001018842 | 0.010528222 | AI965339 | 433 | 69 | 73 | 94 | 7e−37 | ubiquitin-dependent protein |
| | | | | | | | | | | | catabolism |
| 1260 | 2.122977193 | up | 0.000378037 | 0.005596014 | CF922505 | 575 | 86 | 101 | 85 | 5e−46 | small GTPase mediated signal |
| | | | | | | | | | | | transduction |
| 1261 | 2.123498782 | up | 0.000270424 | 0.004600502 | CA800031 | 690 | 76 | 84 | 90 | 2e−39 | translational elongation |
| 1262 | 2.123990714 | up | 0.0000902 | 0.002526814 | CA853339 | 618 | 136 | 199 | 68 | 7e−77 | electron transport |
| 1263 | 2.124128604 | up | 0.000655909 | 0.00796292 | BE806947 | 349 | 14 | 32 | 43 | 5.5 | regulation of transcription |
| 1264 | 2.124974666 | up | 0.000319884 | 0.005076848 | BM567757 | 482 | 70 | 146 | 47 | 2e−33 | |
| 1265 | 2.125648706 | up | 0.003901843 | 0.024935249 | CA802402 | 277 | 8 | 20 | 40 | 3.2 | transport |
| 1266 | 2.125918518 | up | 0.000210686 | 0.003982282 | CF807592 | 568 | 145 | 163 | 88 | 1e−77 | |
| 1267 | 2.126433725 | up | 0.00064272 | 0.007866998 | BU545380 | 645 | 92 | 165 | 55 | 8e−46 | |
| 1268 | 2.127401524 | up | 0.0000978 | 0.002616283 | CD405342 | 672 | 69 | 97 | 71 | 2e−34 | |
| 1269 | 2.127436872 | up | 0.000018 | 0.00121727 | BE346170 | 605 | 111 | 183 | 60 | 5e−68 | xyloglucan biosynthesis |
| 1270 | 2.127786074 | up | 0.0012314 | 0.011791722 | CK606438 | | | | | | |
| 1271 | 2.128110478 | up | 0.000204942 | 0.003909078 | BG839191 | 761 | 98 | 136 | 72 | 3e−46 | regulation of transcription\, DNA- |
| | | | | | | | | | | | dependent |
| 1272 | 2.128170504 | up | 0.000619702 | 0.007697978 | BQ453720 | 253 | 12 | 23 | 52 | 0.059 | |
| 1273 | 2.128260014 | up | 0.001188788 | 0.011560254 | CD409929 | 633 | 65 | 105 | 61 | 7e−35 | electron transport |
| 1274 | 2.12856998 | up | 0.001023904 | 0.01057472 | CD416491 | 449 | 38 | 45 | 84 | 7e−15 | ossification |
| 1275 | 2.128570844 | up | 0.000550939 | 0.007101791 | BI786694 | 541 | 24 | 38 | 63 | 1e−10 | |
| 1276 | 2.128707518 | up | 0.0000934 | 0.002553736 | BE657517 | 730 | 69 | 80 | 86 | 2e−37 | |
| 1277 | 2.128896028 | up | 0.00031215 | 0.005011184 | BM524482 | 576 | 148 | 191 | 77 | 3e−83 | steroid biosynthesis |
| 1278 | 2.129161981 | up | 0.000141726 | 0.003161492 | AW704957 | 300 | 27 | 38 | 71 | 1e−10 | |
| 1279 | 2.130325283 | up | 0.000669425 | 0.008042187 | AI495608 | 524 | 133 | 172 | 77 | 4e−71 | proteolysis and peptidolysis |
| 1280 | 2.130423017 | up | 0.0012017 | 0.01163468 | BM143921 | 494 | 37 | 87 | 42 | 1e−13 | metabolism |
| 1281 | 2.130518481 | up | 0.000499282 | 0.00666805 | AW706444 | 402 | 80 | 136 | 58 | 1e−34 | |
| 1282 | 2.130767941 | up | 0.0008362 | 0.009256276 | CK605866 | 792 | 170 | 200 | 85 | 3e−95 | |
| 1283 | 2.131396058 | up | 0.000498538 | 0.006663827 | AW133014 | 452 | 71 | 124 | 57 | 6e−32 | protein folding |
| 1284 | 2.13139823 | up | 0.002534052 | 0.018811099 | BI699480 | 555 | 86 | 166 | 51 | 2e−39 | regulation of transcription |
| 1285 | 2.131495136 | up | 0.000906756 | 0.009722559 | CD402631 | 509 | 18 | 47 | 38 | 0.70 | |
| 1286 | 2.13152232 | up | 0.0000239 | 0.00133979 | AW780720 | 221 | 37 | 70 | 52 | 3e−13 | |
| 1287 | 2.131654866 | up | 0.0000156 | 0.001135545 | BF426017 | 422 | 68 | 79 | 86 | 2e−34 | |
| 1288 | 2.131964202 | up | 0.001066114 | 0.010838729 | CD390440 | 440 | 32 | 67 | 47 | 6e−10 | cell differentiation |
| 1289 | 2.132156455 | up | 0.002446676 | 0.018391143 | BE556441 | 357 | 46 | 48 | 95 | 4e−20 | |
| 1290 | 2.132783707 | up | 0.0000608 | 0.002077588 | AW831940 | 616 | 124 | 205 | 60 | 2e−69 | |
| 1291 | 2.133117556 | up | 0.0000433 | 0.001770252 | BE329987 | 439 | 18 | 26 | 69 | 4e−06 | |
| 1292 | 2.133302403 | up | 0.001695001 | 0.014453812 | BG839191 | 761 | 98 | 136 | 72 | 3e−46 | regulation of transcription\, DNA- |
| | | | | | | | | | | | dependent |
| 1293 | 2.134158277 | up | 0.000182986 | 0.003668883 | BG839744 | 479 | 93 | 149 | 62 | 2e−50 | tricarboxylic acid cycle |
| 1294 | 2.134790421 | up | 0.000281182 | 0.004713407 | BG154906 | 266 | 20 | 59 | 33 | 0.65 | protein amino acid phosphorylation |
| 1295 | 2.135193068 | up | 0.000211323 | 0.003990314 | BU545471 | 500 | 47 | 52 | 90 | 6e−21 | |
| 1296 | 2.135386908 | up | 0.000943415 | 0.009980826 | BQ610343 | 230 | 10 | 22 | 45 | 3.2 | |
| 1297 | 2.135436861 | up | 0.000699813 | 0.008290479 | CF807225 | 542 | 133 | 147 | 90 | 1e−75 | sex determination |
| 1298 | 2.135500151 | up | 0.000368896 | 0.005501848 | CD394365 | 369 | 26 | 29 | 89 | 2e−09 | protein biosynthesis |
| 1299 | 2.135707775 | up | 0.000776105 | 0.008870381 | BM309579 | 574 | 136 | 192 | 70 | 5e−70 | |
| 1300 | 2.135786377 | up | 0.0000461 | 0.001810721 | BM732455 | 568 | 102 | 119 | 85 | 7e−56 | protein-nucleus import |
| 1301 | 2.136197006 | up | 0.001040502 | 0.010675244 | BI700802 | 564 | 117 | 165 | 70 | 7e−67 | |
| 1302 | 2.136636429 | up | 0.00000862 | 0.000900892 | BM086492 | 463 | 65 | 150 | 43 | 9e−28 | |
| 1303 | 2.137011276 | up | 0.00012585 | 0.002973508 | BU543994 | 459 | 21 | 85 | 24 | 2.8 | |
| 1304 | 2.138716218 | up | 0.001461599 | 0.013099023 | BQ627819 | 229 | 12 | 22 | 54 | 4.2 | |
| 1305 | 2.139091523 | up | 0.00000895 | 0.00091778 | BG551366 | 233 | 9 | 22 | 40 | 9.3 | |
| 1306 | 2.139559092 | up | 0.000672778 | 0.008061763 | BU081672 | 514 | 70 | 139 | 50 | 4e−38 | one-carbon compound metabolism |
| 1307 | 2.13965075 | up | 0.0000492 | 0.001873618 | BI316348 | 521 | 121 | 172 | 70 | 7e−68 | |
| 1308 | 2.14021493 | up | 0.000012 | 0.001038114 | BU081474 | 549 | 85 | 133 | 63 | 4e−48 | |
| 1309 | 2.140355923 | up | 0.0004138 | 0.005911387 | CD416202 | 661 | 52 | 93 | 55 | 7e−24 | |
| 1310 | 2.140875197 | up | 0.00000934 | 0.000924406 | BE210984 | 332 | 78 | 110 | 70 | 3e−37 | |
| 1311 | 2.141192763 | up | 0.0000223 | 0.001314393 | BI970830 | 675 | 49 | 90 | 54 | 1e−24 | carbohydrate metabolism |
| 1312 | 2.141337377 | up | 0.00347045 | 0.023034672 | AW570594 | 609 | 178 | 201 | 88 | 3e−99 | BiP isoform B |
| 1313 | 2.141687426 | up | 0.000827659 | 0.009196912 | CD402549 | 475 | 100 | 109 | 91 | 5e−53 | anti-apoptosis |
| 1314 | 2.142837981 | up | 0.000024 | 0.001341839 | AI441396 | 278 | 14 | 36 | 38 | 1.4 | |
| 1315 | 2.143130268 | up | 0.0000234 | 0.001332938 | BU549006 | 502 | 45 | 116 | 38 | 1e−18 | |
| 1316 | 2.143967753 | up | 0.0000373 | 0.001641557 | BQ785638 | 531 | 22 | 43 | 51 | 2e−07 | |
| 1317 | 2.144013606 | up | 0.007324856 | 0.038169949 | CA802041 | 332 | 12 | 38 | 31 | 7.2 | |
| 1318 | 2.144053338 | up | 0.001031757 | 0.010623251 | CD405725 | 686 | 104 | 111 | 93 | 2e−54 | |
| 1319 | 2.144257845 | up | 0.002665119 | 0.019453865 | BI969436 | 713 | 80 | 159 | 50 | 5e−36 | nucleotide metabolism |
| 1320 | 2.144361259 | up | 0.000559926 | 0.007156325 | BU762524 | 542 | 93 | 100 | 93 | 3e−50 | N-terminal protein amino acid |
| | | | | | | | | | | | acetylation |
| 1321 | 2.144842554 | up | 0.001563195 | 0.013696465 | CD412339 | 707 | 133 | 136 | 97 | 6e−69 | nucleosome assembly |
| 1322 | 2.145729413 | up | 0.0000778 | 0.002341624 | BE659128 | 240 | 20 | 61 | 32 | 0.64 | |
| 1323 | 2.145994721 | up | 0.005497928 | 0.031514359 | AW350101 | 640 | 20 | 64 | 31 | 0.16 | |
| 1324 | 2.147056373 | up | 0.000225891 | 0.00414482 | CD400248 | 601 | 18 | 34 | 52 | 0.003 | proteolysis and peptidolysis |
| 1325 | 2.147389701 | up | 0.000525439 | 0.006889789 | BI095192 | 398 | 19 | 29 | 65 | 5e−06 | transport |
| 1326 | 2.148059141 | up | 0.001012331 | 0.010483982 | BI893945 | 417 | 13 | 13 | 100 | 1.8 | |
| 1327 | 2.148213029 | up | 0.001983943 | 0.016034389 | CD405157 | 572 | 45 | 51 | 88 | 3e−23 | transcription |
| 1328 | 2.148671359 | up | 0.000455281 | 0.006314252 | BI967269 | 700 | 49 | 95 | 51 | 1e−16 | |
| 1329 | 2.148987763 | up | 0.000101029 | 0.00266301 | AW459002 | 458 | 119 | 150 | 79 | 7e−68 | transport |
| 1330 | 2.149419064 | up | 0.000321063 | 0.005091259 | CB063337 | 383 | 35 | 65 | 53 | 2e−14 | amino acid transport |
| 1331 | 2.149850884 | up | 0.0000748 | 0.002298176 | BI970934 | 524 | 29 | 64 | 45 | 2e−11 | |
| 1332 | 2.150369932 | up | 0.000381511 | 0.005622468 | BG653340 | 348 | 11 | 15 | 73 | 0.13 | |
| 1333 | 2.150660111 | up | 0.0000339 | 0.001558741 | BE059335 | 403 | 12 | 31 | 38 | 6.0 | |
| 1334 | 2.150999504 | up | 0.001513756 | 0.013425702 | CA852942 | 251 | 21 | 77 | 27 | 9.5 | electron transport |
| 1335 | 2.151045734 | up | 0.001515813 | 0.013437718 | AI941372 | 564 | 20 | 25 | 80 | 5e−06 | |
| 1336 | 2.151065883 | up | 0.001388061 | 0.012712566 | BE058371 | 623 | 25 | 39 | 64 | 3e−10 | antibiotic biosynthesis |
| 1337 | 2.151340517 | up | 0.0000182 | 0.001221934 | BE555267 | 657 | 90 | 170 | 52 | 7e−48 | |
| 1338 | 2.15185512 | up | 0.000371957 | 0.005538707 | CF807099 | 525 | 127 | 174 | 72 | 1e−66 | regulation of transcription\, DNA- |
| | | | | | | | | | | | dependent |
| 1339 | 2.153945353 | up | 0.000555118 | 0.007121488 | AW596532 | 482 | 12 | 41 | 29 | 0.63 | fatty acid metabolism |
| 1340 | 2.15396599 | up | 0.000185469 | 0.003694813 | CD403858 | 216 | 7 | 11 | 63 | 3.2 | oligopeptide transport |
| 1341 | 2.154420556 | up | 0.000190512 | 0.003736146 | CD401412 | 502 | 53 | 99 | 53 | 5e−25 | |
| 1342 | 2.154835976 | up | 0.000347767 | 0.005339087 | BI971791 | 185 | 13 | 30 | 43 | 0.84 | |
| 1343 | 2.155478949 | up | 0.000124897 | 0.002962079 | AW310849 | 391 | 20 | 22 | 90 | 2e−06 | |
| 1344 | 2.155496264 | up | 0.000116099 | 0.002848619 | BE475334 | 430 | 125 | 142 | 88 | 3e−71 | |
| 1345 | 2.155544272 | up | 0.000264657 | 0.004540648 | BE610145 | 396 | 13 | 19 | 68 | 2.0 | |
| 1346 | 2.155602253 | up | 0.000084 | 0.002450372 | BM091639 | 245 | 10 | 18 | 55 | 4.1 | regulation of transcription\, DNA- |
| | | | | | | | | | | | dependent |
| 1347 | 2.155657 | up | 0.000358311 | 0.005427896 | BI425082 | 572 | 78 | 112 | 69 | 8e−42 | |
| 1348 | 2.15582549 | up | 0.0000109 | 0.000982205 | CD409542 | 703 | 98 | 169 | 57 | 9e−52 | regulation of transcription\, DNA- |
| | | | | | | | | | | | dependent |
| 1349 | 2.156127351 | up | 0.0000536 | 0.001968699 | BM091764 | | | | | | |
| 1350 | 2.156642507 | up | 0.002273342 | 0.017494614 | BE807836 | 536 | 54 | 73 | 73 | 1e−25 | |
| 1351 | 2.156848826 | up | 0.000653432 | 0.007946398 | CA953268 | 465 | 61 | 81 | 75 | 2e−31 | regulation of transcription |
| 1352 | 2.157214432 | up | 0.0000883 | 0.002501346 | BQ299210 | 462 | 64 | 83 | 77 | 2e−33 | electron transport |
| 1353 | 2.158714248 | up | 0.000423471 | 0.006008648 | BE474717 | 353 | 54 | 102 | 52 | 1e−29 | antibiotic biosynthesis |
| 1354 | 2.159093586 | up | 0.000101455 | 0.00266489 | CA802006 | 719 | 62 | 99 | 62 | 5e−30 | |
| 1355 | 2.159102462 | up | 0.000441647 | 0.006180098 | CD392491 | 343 | 16 | 23 | 69 | 0.13 | |
| 1356 | 2.159526481 | up | 0.002201772 | 0.017178481 | CA802038 | 303 | 11 | 25 | 44 | 5.5 | |
| 1357 | 2.159543209 | up | 0.000517929 | 0.006830772 | CD411381 | 501 | 43 | 51 | 84 | 8e−18 | |
| 1358 | 2.160779104 | up | 0.000132161 | 0.003047888 | BE058939 | 377 | 29 | 34 | 85 | 5e−11 | |
| 1359 | 2.161274557 | up | 0.001745589 | 0.014750041 | CA801698 | 593 | 67 | 152 | 44 | 4e−33 | |
| 1360 | 2.161360116 | up | 0.003678708 | 0.023952037 | CA801096 | 609 | 149 | 175 | 85 | 1e−82 | acetyl-CoA biosynthesis |
| 1361 | 2.161546067 | up | 0.000338378 | 0.005270291 | BF596983 | 464 | 72 | 117 | 61 | 9e−39 | |
| 1362 | 2.161570668 | up | 0.001179207 | 0.011519691 | BU964411 | 391 | 23 | 66 | 34 | 2.5 | |
| 1363 | 2.161576738 | up | 0.000236173 | 0.004260624 | AW349404 | 613 | 61 | 107 | 57 | 7e−28 | tRNA aminoacylation for protein |
| | | | | | | | | | | | translation |
| 1364 | 2.161895989 | up | 0.0000625 | 0.002103502 | BU964433 | 558 | 101 | 157 | 64 | 2e−58 | |
| 1365 | 2.162416911 | up | 0.000310657 | 0.004993616 | BU550055 | 673 | 76 | 116 | 65 | 6e−42 | |
| 1366 | 2.162674794 | up | 0.0000354 | 0.001589423 | BE821751 | 654 | 68 | 103 | 66 | 7e−32 | |
| 1367 | 2.163002624 | up | 0.0000261 | 0.001380871 | CD412407 | 99 | 10 | 15 | 66 | 2.4 | regulation of transcription\, DNA- |
| | | | | | | | | | | | dependent |
| 1368 | 2.163357948 | up | 0.000626184 | 0.007752887 | AW734949 | 531 | 93 | 134 | 69 | 5e−47 | |
| 1369 | 2.164354603 | up | 0.007629301 | 0.039207668 | BI943454 | 625 | 178 | 206 | 86 | e−103 | selenocysteine incorporation |
| 1370 | 2.164722554 | up | 0.0000185 | 0.00122629 | BF069870 | 518 | 102 | 149 | 68 | 4e−57 | protein biosynthesis |
| 1371 | 2.165594254 | up | 0.000133465 | 0.003064816 | BG650866 | 468 | 70 | 98 | 71 | 3e−36 | |
| 1372 | 2.165787736 | up | 0.001912224 | 0.015619192 | CD412218 | 489 | 70 | 79 | 88 | 6e−37 | protein biosynthesis |
| 1373 | 2.165802312 | up | 0.0000539 | 0.001975394 | BQ081061 | 441 | 34 | 58 | 58 | 5e−13 | |
| 1374 | 2.166077796 | up | 0.000390932 | 0.005719377 | CA820360 | 572 | 38 | 56 | 67 | 7e−19 | |
| 1375 | 2.166150905 | up | 0.00040852 | 0.005867922 | BQ452761 | 242 | 14 | 42 | 33 | 4.1 | |
| 1376 | 2.166179118 | up | 0.003185931 | 0.021854343 | CD393849 | 675 | 55 | 88 | 62 | 5e−26 | |
| 1377 | 2.168099197 | up | 0.000833742 | 0.009245388 | BI786484 | | | | | | |
| 1378 | 2.168225943 | up | 0.000656098 | 0.00796292 | CD400159 | 595 | 15 | 33 | 45 | 4.6 | |
| 1379 | 2.16830239 | up | 0.000187444 | 0.00370587 | CD405347 | 339 | 19 | 20 | 95 | 3e−05 | protein biosynthesis |
| 1380 | 2.16868106 | up | 0.000970631 | 0.010158409 | CD393260 | 705 | 46 | 49 | 93 | 1e−21 | ciliary/flagellar motility |
| 1381 | 2.168746697 | up | 0.00025176 | 0.004418893 | BM143803 | 420 | 18 | 51 | 35 | 0.12 | transport |
| 1382 | 2.168972218 | up | 0.000125215 | 0.002966424 | CD415704 | 687 | 51 | 76 | 67 | 2e−29 | metabolism |
| 1383 | 2.169331494 | up | 0.000136648 | 0.003098179 | CD399802 | 535 | 61 | 75 | 81 | 1e−29 | tricarboxylic acid cycle |
| 1384 | 2.17019458 | up | 0.00031671 | 0.005043386 | BI968899 | 708 | 109 | 157 | 69 | 3e−62 | |
| 1385 | 2.171004479 | up | 0.000316971 | 0.005043386 | CD404692 | 356 | 39 | 49 | 79 | 9e−17 | RNA processing |
| 1386 | 2.171118072 | up | 0.000322962 | 0.005110599 | CD395870 | 340 | 27 | 92 | 29 | 0.17 | |
| 1387 | 2.171207558 | up | 0.000296778 | 0.00485001 | AW350096 | 674 | 111 | 131 | 84 | 5e−63 | |
| 1388 | 2.171216781 | up | 0.000796055 | 0.00897566 | BI973987 | 420 | 23 | 28 | 82 | 8e−09 | electron transport |
| 1389 | 2.171286651 | up | 0.000321562 | 0.005097028 | BU544809 | 552 | 21 | 76 | 27 | 1.8 | |
| 1390 | 2.172017058 | up | 0.00058599 | 0.007406398 | BU550790 | 634 | 85 | 177 | 48 | 2e−38 | |
| 1391 | 2.172379365 | up | 0.000328495 | 0.00516771 | CD396639 | 605 | 20 | 59 | 33 | 3.7 | |
| 1392 | 2.173687187 | up | 0.00158246 | 0.013800751 | BM525482 | 485 | 39 | 45 | 86 | 1e−16 | |
| 1393 | 2.173781647 | up | 0.000196066 | 0.003797545 | BE611495 | 480 | 63 | 92 | 68 | 4e−33 | |
| 1394 | 2.174076687 | up | 0.000250945 | 0.004410765 | BQ453969 | 382 | 25 | 99 | 25 | 3.1 | regulation of transcription\, DNA- |
| | | | | | | | | | | | dependent |
| 1395 | 2.174157471 | up | 0.00243157 | 0.018307757 | BG511220 | 297 | 12 | 32 | 37 | 4.2 | |
| 1396 | 2.17417577 | up | 0.000134874 | 0.003077454 | BI498494 | 515 | 97 | 140 | 69 | 3e−54 | threonine catabolism |
| 1397 | 2.174760746 | up | 0.000154456 | 0.003322585 | BM188514 | 594 | 65 | 153 | 42 | 2e−30 | |
| 1398 | 2.174800614 | up | 0.000102475 | 0.002675093 | BM884979 | 533 | 78 | 112 | 69 | 2e−41 | |
| 1399 | 2.175318731 | up | 0.002434773 | 0.018323638 | AW348858 | 714 | 99 | 112 | 88 | 4e−55 | |
| 1400 | 2.175363545 | up | 0.002405187 | 0.018148186 | BI471357 | 329 | 7 | 17 | 41 | 3.3 | |
| 1401 | 2.175637753 | up | 0.0000915 | 0.002541909 | CD392612 | 654 | 76 | 135 | 56 | 1e−32 | G-protein coupled receptor protein |
| | | | | | | | | | | | signaling pathway |
| 1402 | 2.176013201 | up | 0.00659136 | 0.035662747 | CD399306 | 646 | 113 | 150 | 75 | 8e−60 | protein transport |
| 1403 | 2.176208666 | up | 0.0000673 | 0.002177084 | BE821972 | 502 | 79 | 82 | 96 | 2e−38 | ATP biosynthesis |
| 1404 | 2.176508663 | up | 0.001055258 | 0.01076671 | BE347555 | 617 | 99 | 169 | 58 | 7e−52 | |
| 1405 | 2.177072472 | up | 0.004414116 | 0.027181377 | BI498562 | 477 | 58 | 74 | 78 | 2e−30 | |
| 1406 | 2.17809703 | up | 0.000897964 | 0.009680247 | CD408714 | 599 | 52 | 58 | 89 | 1e−23 | glycolysis |
| 1407 | 2.178892917 | up | 0.000158432 | 0.003355218 | BI786260 | 486 | 63 | 143 | 44 | 4e−19 | |
| 1408 | 2.178953807 | up | 0.0000894 | 0.002514157 | CD402040 | 369 | 21 | 73 | 28 | 1.7 | |
| 1409 | 2.179391933 | up | 0.000686674 | 0.008194336 | AW202211 | 319 | 70 | 82 | 85 | 2e−35 | |
| 1410 | 2.179699714 | up | 0.001496515 | 0.013319908 | BQ253626 | 440 | 86 | 117 | 73 | 2e−39 | transport |
| 1411 | 2.17980548 | up | 0.000157859 | 0.003347889 | BI701744 | 564 | 78 | 86 | 90 | 3e−38 | fatty acid biosynthesis |
| 1412 | 2.181234945 | up | 0.0000822 | 0.002420756 | BM086300 | 358 | 27 | 43 | 62 | 4e−11 | |
| 1413 | 2.181269416 | up | 0.003195756 | 0.021859459 | CD398574 | 441 | 24 | 55 | 43 | 2e−09 | carbohydrate metabolism |
| 1414 | 2.181300644 | up | 0.001240699 | 0.011847593 | BG046039 | 488 | 122 | 161 | 75 | 3e−70 | selenocysteine incorporation |
| 1415 | 2.181500842 | up | 0.000222445 | 0.004111572 | BI969568 | 775 | 53 | 83 | 63 | 1e−20 | |
| 1416 | 2.18169766 | up | 0.000018 | 0.001219768 | BQ785421 | 591 | 159 | 190 | 83 | 1e−90 | |
| 1417 | 2.181921987 | up | 0.0000549 | 0.001984819 | CK606265 | 775 | 129 | 188 | 68 | 4e−73 | phosphoenolpyruvate-dependent |
| | | | | | | | | | | | sugar phosphotransferase system |
| 1418 | 2.182046296 | up | 0.007804609 | 0.039810491 | BM085604 | 565 | 136 | 190 | 71 | 2e−75 | |
| 1419 | 2.184100154 | up | 0.001816186 | 0.015120416 | AW396379 | 226 | 15 | 44 | 34 | 1.9 | |
| 1420 | 2.184373561 | up | 0.000148693 | 0.003246051 | CD392800 | 579 | 18 | 24 | 75 | 9e−06 | protein folding |
| 1421 | 2.18518246 | up | 0.000167083 | 0.003478297 | CA799618 | 431 | 39 | 91 | 42 | 6e−09 | protein amino acid phosphorylation |
| 1422 | 2.185411293 | up | 0.000517302 | 0.006827294 | BQ297771 | 468 | 117 | 154 | 75 | 8e−65 | |
| 1423 | 2.18542795 | up | 0.000605542 | 0.007577136 | BE210621 | 516 | 122 | 170 | 71 | 8e−66 | |
| 1424 | 2.185978549 | up | 0.009897472 | 0.046713488 | BF324269 | 563 | 36 | 61 | 59 | 1e−12 | |
| 1425 | 2.186799486 | up | 0.000398347 | 0.005789573 | BM527373 | 435 | 46 | 94 | 48 | 5e−15 | |
| 1426 | 2.186873204 | up | 0.000224712 | 0.004134189 | BU763563 | 348 | 19 | 23 | 82 | 0.007 | transport |
| 1427 | 2.186950644 | up | 0.000166807 | 0.003476062 | BM731611 | | | | | | |
| 1428 | 2.186984894 | up | 0.001267973 | 0.012028633 | BE658080 | 731 | 86 | 95 | 90 | 2e−44 | protein biosynthesis |
| 1429 | 2.187317567 | up | 0.0000144 | 0.00111964 | CD411996 | 602 | 84 | 99 | 84 | 3e−43 | selenocysteine incorporation |
| 1430 | 2.188278339 | up | 0.000313763 | 0.005022772 | CD401994 | 513 | 61 | 88 | 69 | 1e−31 | |
| 1431 | 2.188353517 | up | 0.002224159 | 0.017287137 | BG508287 | 514 | 15 | 45 | 33 | 1.2 | |
| 1432 | 2.189040318 | up | 0.000095 | 0.002566638 | AW348696 | 713 | 115 | 174 | 66 | 4e−60 | nucleotide-excision repair |
| 1433 | 2.189410118 | up | 0.000501674 | 0.006684362 | BI893945 | 417 | 13 | 13 | 100 | 1.8 | |
| 1434 | 2.189794854 | up | 0.002368262 | 0.017966882 | CD418023 | 587 | 73 | 79 | 92 | 1e−36 | transport |
| 1435 | 2.191286358 | up | 0.000179814 | 0.003640408 | CD410644 | 629 | 21 | 66 | 31 | 0.055 | protein amino acid phosphorylation |
| 1436 | 2.191297343 | up | 0.00103498 | 0.010645265 | CD396616 | 544 | 41 | 65 | 63 | 8e−17 | histidine biosynthesis |
| 1437 | 2.191353642 | up | 0.000553628 | 0.007107221 | AW394731 | 391 | 48 | 63 | 76 | 2e−19 | |
| 1438 | 2.192491544 | up | 0.0000475 | 0.001830505 | BI945865 | 495 | 101 | 166 | 60 | 3e−59 | carbohydrate metabolism |
| 1439 | 2.192794149 | up | 0.002749955 | 0.019853513 | BU551333 | 579 | 35 | 52 | 67 | 3e−12 | |
| 1440 | 2.192803076 | up | 0.0000345 | 0.001564445 | BE823966 | 652 | 90 | 134 | 67 | 1e−46 | |
| 1441 | 2.194946721 | up | 0.001864393 | 0.015339183 | CD417045 | 537 | 18 | 63 | 28 | 3.0 | |
| 1442 | 2.19531973 | up | 0.000840278 | 0.009287751 | CD399092 | 680 | 43 | 49 | 87 | 4e−19 | response to light |
| 1443 | 2.195657859 | up | 0.000643533 | 0.007874381 | BM139971 | | | | | | |
| 1444 | 2.195720476 | up | 0.0000718 | 0.002248657 | AW830642 | 555 | 119 | 187 | 63 | 1e−65 | intracellular protein transport |
| 1445 | 2.19586791 | up | 0.0000712 | 0.002241939 | BU764116 | 608 | 38 | 91 | 41 | 6e−14 | |
| 1446 | 2.196410447 | up | 0.003566081 | 0.023500696 | AW101759 | 375 | 38 | 67 | 56 | 2e−20 | |
| 1447 | 2.196653845 | up | 0.001375897 | 0.012647356 | CD414364 | 686 | 121 | 191 | 63 | 4e−67 | phosphoenolpyruvate-dependent |
| | | | | | | | | | | | sugar phosphotransferase system |
| 1448 | 2.196978311 | up | 0.000245638 | 0.00436027 | BU549753 | 421 | 73 | 100 | 73 | 2e−38 | |
| 1449 | 2.197622717 | up | 0.003910483 | 0.024958879 | BG155103 | | | | | | |
| 1450 | 2.198506444 | up | 0.00568758 | 0.032252769 | BM094054 | 576 | 46 | 52 | 88 | 2e−22 | regulation of translation |
| 1451 | 2.198875276 | up | 0.00017383 | 0.003561249 | BM523920 | 554 | 69 | 113 | 61 | 6e−34 | |
| 1452 | 2.198918846 | up | 0.0000567 | 0.002008532 | BG551218 | 429 | 52 | 131 | 39 | 2e−14 | |
| 1453 | 2.199407799 | up | 0.000719895 | 0.008456636 | CF806335 | 674 | 106 | 191 | 55 | 4e−47 | |
| 1454 | 2.199525795 | up | 0.000291385 | 0.00479656 | CD394784 | 452 | 13 | 15 | 86 | 0.085 | ‘de novo’ pyrimidine base |
| | | | | | | | | | | | biosynthesis |
| 1455 | 2.200613633 | up | 0.000218689 | 0.004072519 | AW317407 | 527 | 31 | 76 | 40 | 4e−12 | |
| 1456 | 2.200645247 | up | 0.00171536 | 0.014554825 | CD391962 | 666 | 149 | 155 | 96 | 4e−81 | |
| 1457 | 2.200869724 | up | 0.000307229 | 0.004953324 | CD405725 | 686 | 104 | 111 | 93 | 2e−54 | |
| 1458 | 2.201442208 | up | 0.0108614 | 0.049749233 | CF808733 | 588 | 26 | 110 | 23 | 1.2 | tRNA processing |
| 1459 | 2.201476694 | up | 0.006074781 | 0.033721488 | BE347941 | 447 | 33 | 62 | 53 | 3e−11 | |
| 1460 | 2.201946196 | up | 0.000327624 | 0.005161333 | AW311291 | | | | | | |
| 1461 | 2.201971854 | up | 0.000192065 | 0.003750952 | CK605868 | 664 | 201 | 220 | 91 | e−116 | methionine synthase, methionine |
| | | | | | | | | | | | biosynthesis |
| 1462 | 2.202062587 | up | 0.0000275 | 0.001422797 | AI938121 | 411 | 10 | 24 | 41 | 4.9 | |
| 1463 | 2.202362435 | up | 0.000104736 | 0.002706873 | CD399388 | 471 | 52 | 61 | 85 | 4e−27 | |
| 1464 | 2.202552443 | up | 0.000852089 | 0.00937064 | BE822328 | 784 | 95 | 145 | 66 | 2e−50 | |
| 1465 | 2.20261687 | up | 0.000246827 | 0.004373129 | CF807491 | 681 | 73 | 108 | 67 | 7e−36 | glycolysis |
| 1466 | 2.202895713 | up | 0.000375619 | 0.005575587 | CD402446 | 423 | 23 | 53 | 43 | 0.61 | |
| 1467 | 2.202915917 | up | 0.002207983 | 0.017212659 | BG043416 | 656 | 99 | 211 | 46 | 2e−48 | multidrug transport |
| 1468 | 2.203391872 | up | 0.00035835 | 0.005427896 | BE800135 | 486 | 84 | 158 | 53 | 5e−39 | |
| 1469 | 2.204696761 | up | 0.000213537 | 0.004015994 | BE821922 | 475 | 28 | 55 | 50 | 5e−05 | regulation of transcription\, DNA- |
| | | | | | | | | | | | dependent |
| 1470 | 2.205211475 | up | 0.00000981 | 0.000945991 | BE806961 | 505 | 37 | 57 | 64 | 6e−16 | transport |
| 1471 | 2.205560833 | up | 0.002112554 | 0.016721715 | AF244890.1 | | | | | | receptor-like protein kinase 3 |
| 1472 | 2.205814331 | up | 0.002004866 | 0.016161815 | BG047189 | 625 | 137 | 194 | 70 | 4e−74 | regulation of transcription\, DNA- |
| | | | | | | | | | | | dependent |
| 1473 | 2.205835858 | up | 0.0000366 | 0.001624759 | AW309611 | 512 | 26 | 57 | 45 | 7e−06 | |
| 1474 | 2.207964365 | up | 0.000147731 | 0.00323444 | BU091479 | 379 | 17 | 27 | 62 | 3e−04 | |
| 1475 | 2.208259293 | up | 0.001366817 | 0.012604279 | CD404670 | 685 | 110 | 205 | 53 | 2e−55 | glycolysis |
| 1476 | 2.20835005 | up | 0.000185914 | 0.003694813 | CD408574 | 635 | 74 | 158 | 46 | 1e−34 | protein amino acid phosphorylation |
| 1477 | 2.208793741 | up | 0.0000739 | 0.002284101 | BM188020 | 547 | 49 | 100 | 49 | 1e−24 | protein biosynthesis |
| 1478 | 2.208995959 | up | 0.001559358 | 0.013684794 | BF067955 | 350 | 31 | 43 | 72 | 6e−13 | protein biosynthesis |
| 1479 | 2.20918354 | up | 0.000184451 | 0.003688422 | BE819998 | 827 | 72 | 160 | 45 | 1e−24 | |
| 1480 | 2.209234367 | up | 0.000248572 | 0.004391641 | CD395933 | 633 | 101 | 132 | 76 | 5e−48 | |
| 1481 | 2.209540388 | up | 0.002047237 | 0.016384686 | BG652905 | 386 | 16 | 44 | 36 | 0.076 | |
| 1482 | 2.209861325 | up | 0.001011379 | 0.010481464 | BU545380 | 645 | 92 | 165 | 55 | 8e−46 | |
| 1483 | 2.210119415 | up | 0.000549767 | 0.007089108 | CA799510 | 422 | 21 | 67 | 31 | 0.055 | |
| 1484 | 2.210634965 | up | 0.000288431 | 0.004773484 | CD405217 | 501 | 17 | 54 | 31 | 2.0 | leucine catabolism |
| 1485 | 2.210788229 | up | 0.0000971 | 0.00260077 | CD396488 | 605 | 10 | 34 | 29 | 8.2 | |
| 1486 | 2.211205013 | up | 0.000123137 | 0.002948224 | CF806184 | 476 | 88 | 136 | 64 | 1e−43 | glucose catabolism |
| 1487 | 2.211255297 | up | 0.000146657 | 0.003216456 | BE555690 | 430 | 44 | 113 | 38 | 5e−12 | |
| 1488 | 2.211407632 | up | 0.00018858 | 0.003715696 | BG881363 | 461 | 94 | 125 | 75 | 2e−49 | protein amino acid phosphorylation |
| 1489 | 2.212265448 | up | 0.000173556 | 0.003557561 | CD391001 | 666 | 114 | 151 | 75 | 2e−62 | protein biosynthesis |
| 1490 | 2.212922793 | up | 0.003066652 | 0.021289971 | BE209869 | 401 | 71 | 88 | 80 | 6e−37 | |
| 1491 | 2.213067811 | up | 0.0000606 | 0.002073048 | CF807592 | 568 | 145 | 163 | 88 | 1e−77 | |
| 1492 | 2.21311228 | up | 0.00046274 | 0.006373439 | CF806590 | 668 | 161 | 222 | 72 | 2e−94 | calnexin, G-protein coupled receptor |
| | | | | | | | | | | | protein signaling pathway |
| 1493 | 2.213446839 | up | 0.001377871 | 0.012653139 | AW310813 | 353 | 17 | 32 | 53 | 0.004 | two-component signal transduction |
| | | | | | | | | | | | system (phosphorelay) |
| 1494 | 2.213478716 | up | 0.000066 | 0.002161506 | BU546487 | 626 | 93 | 148 | 62 | 2e−48 | |
| 1495 | 2.213588489 | up | 0.00000609 | 0.000832282 | BI942288 | 538 | 119 | 178 | 66 | 8e−62 | arginine biosynthesis |
| 1496 | 2.213805273 | up | 0.0000778 | 0.002341624 | AW832138 | 454 | 106 | 151 | 70 | 9e−55 | protein folding |
| 1497 | 2.213994151 | up | 0.000380656 | 0.005614894 | CA819143 | 445 | 11 | 31 | 35 | 9.9 | |
| 1498 | 2.214034578 | up | 0.000870346 | 0.009475313 | BM307497 | 402 | 48 | 59 | 81 | 2e−23 | |
| 1499 | 2.214221647 | up | 0.0000588 | 0.002038083 | BE821662 | 704 | 63 | 106 | 59 | 9e−31 | phenylalanyl-tRNA aminoacylation |
| 1500 | 2.214329401 | up | 0.002375778 | 0.018013001 | CD416390 | 534 | 32 | 44 | 72 | 3e−14 | |
| 1501 | 2.215488701 | up | 0.001968472 | 0.01594017 | AW508309 | 226 | 14 | 48 | 29 | 1.1 | metal ion transport |
| 1502 | 2.21553362 | up | 0.000121344 | 0.002923918 | CF807754 | 570 | 86 | 167 | 51 | 1e−44 | |
| 1503 | 2.215621648 | up | 0.000129218 | 0.003021647 | BG046739 | 406 | 46 | 83 | 55 | 1e−22 | |
| 1504 | 2.216012137 | up | 0.000436639 | 0.006121403 | BQ297216 | 584 | 30 | 35 | 85 | 1e−10 | glycolysis |
| 1505 | 2.216201697 | up | 0.000119891 | 0.002901929 | CD415130 | 674 | 82 | 158 | 51 | 2e−37 | regulation of transcription |
| 1506 | 2.216358309 | up | 0.00023847 | 0.004287665 | BG839191 | 761 | 98 | 136 | 72 | 3e−46 | regulation of transcription\, DNA- |
| | | | | | | | | | | | dependent |
| 1507 | 2.216610347 | up | 0.0000888 | 0.002506437 | CD407652 | 546 | 72 | 86 | 83 | 2e−34 | |
| 1508 | 2.21704365 | up | 0.000737149 | 0.008594005 | CD408525 | 463 | 15 | 21 | 71 | 8e−06 | |
| 1509 | 2.217365062 | up | 0.000109066 | 0.002775649 | BQ298988 | 160 | 12 | 32 | 37 | 2.4 | |
| 1510 | 2.217649298 | up | 0.0000921 | 0.002547532 | BI317354 | 377 | 28 | 32 | 87 | 1e−10 | |
| 1511 | 2.218710125 | up | 0.0000141 | 0.001112691 | CA799909 | 408 | 55 | 121 | 45 | 5e−24 | nucleosome assembly |
| 1512 | 2.219226947 | up | 0.000143363 | 0.003181853 | BG652101 | 396 | 12 | 27 | 44 | 7.5 | |
| 1513 | 2.219236011 | up | 0.00049341 | 0.00663774 | CF807858 | 517 | 66 | 77 | 85 | 6e−34 | |
| 1514 | 2.22007774 | up | 0.001152606 | 0.011358168 | BI969225 | 633 | 11 | 28 | 39 | 0.21 | nucleotide-excision repair |
| 1515 | 2.220246978 | up | 0.000662957 | 0.00800528 | CA802910 | | | | | | Isolate x138 magmas-like protein |
| | | | | | | | | | | | mRNA, complete cds; nuclear gene |
| | | | | | | | | | | | for mitochondrial product |
| 1516 | 2.22050335 | up | 0.0000533 | 0.001959669 | CA802695 | 411 | 11 | 27 | 40 | 0.20 | |
| 1517 | 2.220619771 | up | 0.0000424 | 0.001755385 | CD395788 | 666 | 79 | 158 | 50 | 1e−31 | |
| 1518 | 2.221316865 | up | 0.0000905 | 0.002529045 | CD414658 | 593 | 59 | 149 | 39 | 1e−22 | |
| 1519 | 2.221427548 | up | 0.0000341 | 0.00156033 | BU547088 | 644 | 17 | 20 | 85 | 0.003 | electron transport |
| 1520 | 2.221603859 | up | 0.008985108 | 0.043755289 | BE021552 | 529 | 98 | 165 | 59 | 3e−54 | |
| 1521 | 2.223324402 | up | 0.000312294 | 0.005011365 | BE820895 | 726 | 65 | 93 | 69 | 6e−32 | nuclear membrane fusion |
| 1522 | 2.22361172 | up | 0.002306015 | 0.017647702 | CD402749 | 570 | 63 | 71 | 88 | 3e−29 | 40S ribosomal protein S25 |
| 1523 | 2.223729801 | up | 0.000364133 | 0.005472037 | BM091812 | 294 | 9 | 18 | 50 | 4.2 | |
| 1524 | 2.224236117 | up | 0.000116033 | 0.002848619 | BM143354 | 323 | 14 | 64 | 21 | 2.4 | |
| 1525 | 2.224748226 | up | 0.000260498 | 0.004496733 | CD393359 | 508 | 77 | 91 | 84 | 1e−39 | |
| 1526 | 2.225076058 | up | 0.009179334 | 0.044430763 | BM270487 | 534 | 42 | 73 | 57 | 1e−19 | |
| 1527 | 2.225110363 | up | 0.000126086 | 0.002974387 | BQ611191 | 436 | 39 | 68 | 57 | 1e−13 | |
| 1528 | 2.225114146 | up | 0.000863539 | 0.009442199 | BE823700 | 573 | 66 | 104 | 63 | 3e−29 | ubiquitin-dependent protein |
| | | | | | | | | | | | catabolism |
| 1529 | 2.226049502 | up | 0.000240739 | 0.004304359 | AW350941 | 618 | 80 | 91 | 87 | 1e−40 | |
| 1530 | 2.226186353 | up | 0.0000271 | 0.001411072 | BI316736 | 418 | 113 | 139 | 81 | 3e−65 | proteolysis and peptidolysis |
| 1531 | 2.22624712 | up | 0.0000343 | 0.001562658 | BF008821 | 466 | 35 | 61 | 57 | 5e−16 | regulation of nitrogen utilization |
| 1532 | 2.226467488 | up | 0.001634865 | 0.014104082 | CD411974 | 629 | 38 | 89 | 42 | 3e−09 | |
| 1533 | 2.226635057 | up | 0.00029365 | 0.004821109 | BE657216 | 758 | 23 | 37 | 62 | 2e−06 | translational initiation |
| 1534 | 2.227413136 | up | 0.000260374 | 0.004496644 | BQ297083 | 526 | 19 | 29 | 65 | 5e−05 | |
| 1535 | 2.227935838 | up | 0.0000176 | 0.001209042 | BE583628 | 682 | 99 | 114 | 86 | 6e−56 | electron transport |
| 1536 | 2.227982408 | up | 0.000525529 | 0.006889789 | BU546934 | 645 | 70 | 107 | 65 | 7e−35 | cell wall catabolism |
| 1537 | 2.228100434 | up | 0.000293773 | 0.004821109 | CF809274 | 532 | 73 | 93 | 78 | 2e−38 | protein biosynthesis |
| 1538 | 2.228914449 | up | 0.00000772 | 0.000894724 | AW185201 | 223 | 11 | 35 | 31 | 5.5 | |
| 1539 | 2.228995273 | up | 0.0000186 | 0.00122632 | BI893673 | 444 | 43 | 57 | 75 | 2e−21 | |
| 1540 | 2.229079847 | up | 0.000267991 | 0.004577758 | BM188504 | 617 | 128 | 172 | 74 | 1e−67 | transport |
| 1541 | 2.229368738 | up | 0.000250109 | 0.004407052 | BM092172 | 575 | 82 | 121 | 67 | 2e−45 | |
| 1542 | 2.22942001 | up | 0.0000301 | 0.001477923 | BU549340 | 628 | 65 | 124 | 52 | 1e−31 | |
| 1543 | 2.2299095 | up | 0.000402363 | 0.005825437 | BG238637 | 590 | 23 | 36 | 63 | 1e−08 | anion transport |
| 1544 | 2.230165157 | up | 0.000557718 | 0.007140238 | CA783243 | 455 | 107 | 128 | 83 | 2e−56 | response to cold |
| 1545 | 2.230316369 | up | 0.00101077 | 0.010479352 | AW310199 | 655 | 103 | 131 | 78 | 3e−55 | signal transduction |
| 1546 | 2.230470108 | up | 0.0000338 | 0.001558741 | AW760424 | 406 | 18 | 27 | 66 | 2e−04 | |
| 1547 | 2.231121672 | up | 0.0000245 | 0.001353718 | BM732761 | 600 | 177 | 199 | 88 | e−105 | main pathways of carbohydrate |
| | | | | | | | | | | | metabolism |
| 1548 | 2.23122529 | up | 0.000898178 | 0.009680247 | AW310310 | 681 | 82 | 111 | 73 | 1e−42 | fatty acid biosynthesis |
| 1549 | 2.231927768 | up | 0.001828988 | 0.015184457 | CD393504 | 505 | 25 | 50 | 50 | 4e−09 | tRNA aminoacylation for protein |
| | | | | | | | | | | | translation |
| 1550 | 2.232876217 | up | 0.001707657 | 0.01451566 | BE659765 | 361 | 8 | 16 | 50 | 1.2 | fatty acid biosynthesis |
| 1551 | 2.232972714 | up | 0.0000137 | 0.001103751 | BM178502 | 420 | 22 | 31 | 70 | 1e−06 | dicarboxylic acid transport |
| 1552 | 2.233006391 | up | 0.00165579 | 0.014225879 | BG047180 | 469 | 54 | 74 | 72 | 6e−27 | |
| 1553 | 2.233047108 | up | 0.000644283 | 0.00788099 | CD406505 | 501 | 11 | 19 | 57 | 1.2 | |
| 1554 | 2.235804276 | up | 0.000639847 | 0.007844586 | CD414276 | 609 | 37 | 45 | 82 | 4e−15 | protein biosynthesis |
| 1555 | 2.23583796 | up | 0.0000297 | 0.00147316 | CA819637 | 356 | 9 | 22 | 40 | 1.5 | |
| 1556 | 2.236317397 | up | 0.0000681 | 0.002184143 | BM893460 | 169 | 14 | 46 | 30 | 0.045 | |
| 1557 | 2.236819373 | up | 0.000176767 | 0.0036018 | BQ298949 | 367 | 24 | 28 | 85 | 4e−09 | protein biosynthesis |
| 1558 | 2.236834729 | up | 0.000487108 | 0.006585943 | BM091810 | 243 | 17 | 47 | 36 | 1.1 | amino acid transport |
| 1559 | 2.23762793 | up | 0.002241188 | 0.017342117 | AW317215 | 486 | 18 | 49 | 36 | 0.84 | G-protein coupled receptor protein |
| | | | | | | | | | | | signaling pathway |
| 1560 | 2.237769977 | up | 0.000458139 | 0.006335459 | CD407817 | 657 | 92 | 152 | 60 | 3e−49 | |
| 1561 | 2.237855922 | up | 0.000033 | 0.001543897 | CF808392 | 748 | 111 | 140 | 79 | 1e−60 | aspartate metabolism |
| 1562 | 2.238065306 | up | 0.000348567 | 0.005341571 | BM521713 | 544 | 157 | 180 | 87 | 1e−87 | methionine biosynthesis |
| 1563 | 2.238377935 | up | 0.002049611 | 0.016384686 | BI321473 | 279 | 11 | 23 | 47 | 5.4 | |
| 1564 | 2.239198021 | up | 0.000089 | 0.002506437 | CD392158 | 704 | 99 | 156 | 63 | 2e−52 | |
| 1565 | 2.23935835 | up | 0.000492841 | 0.006632463 | BM270865 | 458 | 107 | 153 | 69 | 9e−63 | carbohydrate metabolism |
| 1566 | 2.239473085 | up | 0.000631706 | 0.007798161 | BU764584 | 450 | 33 | 40 | 82 | 4e−12 | |
| 1567 | 2.240332442 | up | 0.00063091 | 0.007793451 | BU548254 | 612 | 28 | 73 | 38 | 0.008 | |
| 1568 | 2.240485182 | up | 0.0000669 | 0.002172462 | CF807843 | 544 | 128 | 180 | 71 | 3e−73 | |
| 1569 | 2.24086786 | up | 0.000251959 | 0.004420006 | BE660057 | 703 | 31 | 117 | 26 | 0.029 | |
| 1570 | 2.241608893 | up | 0.000110706 | 0.002790944 | BI321807 | 435 | 48 | 55 | 87 | 7e−29 | regulation of transcription\, DNA- |
| | | | | | | | | | | | dependent |
| 1571 | 2.241617299 | up | 0.0001719 | 0.003537122 | BM954427 | | | | | | |
| 1572 | 2.242324232 | up | 0.003693746 | 0.024006438 | BI786698 | 549 | 12 | 27 | 44 | 8.9 | |
| 1573 | 2.242379342 | up | 0.000662607 | 0.008004703 | BE021139 | 379 | 13 | 28 | 46 | 0.21 | |
| 1574 | 2.242521881 | up | 0.000271198 | 0.004607433 | AW100676 | 367 | 32 | 34 | 94 | 6e−13 | |
| 1575 | 2.242618813 | up | 0.0000915 | 0.002541909 | CD395886 | 541 | 71 | 112 | 63 | 3e−37 | |
| 1576 | 2.242648759 | up | 0.000251149 | 0.004412297 | CD412731 | 474 | 23 | 26 | 88 | 3e−07 | regulation of translation |
| 1577 | 2.242777167 | up | 0.00042034 | 0.005983014 | BG551351 | 471 | 87 | 103 | 84 | 4e−49 | |
| 1578 | 2.243806401 | up | 0.001182215 | 0.011528401 | BG043801 | 447 | 64 | 155 | 41 | 2e−20 | |
| 1579 | 2.243996491 | up | 0.000234879 | 0.004253608 | BF595393 | 488 | 19 | 32 | 59 | 2e−04 | |
| 1580 | 2.244522981 | up | 0.001761881 | 0.014855726 | BF070459 | 615 | 133 | 193 | 68 | 2e−72 | cell differentiation |
| 1581 | 2.244743032 | up | 0.000255204 | 0.004446154 | BE190261 | 353 | 10 | 18 | 55 | 4.3 | protein amino acid phosphorylation |
| 1582 | 2.2448254 | up | 0.000156289 | 0.003340013 | AW156783 | 394 | 14 | 30 | 46 | 0.68 | protein amino acid phosphorylation |
| 1583 | 2.245074131 | up | 0.000425978 | 0.006032651 | CD404243 | 617 | 49 | 82 | 59 | 1e−19 | |
| 1584 | 2.245331743 | up | 0.001233958 | 0.011804199 | BU544114 | 597 | 60 | 100 | 60 | 7e−30 | |
| 1585 | 2.245671633 | up | 0.000156372 | 0.003340013 | AW310123 | 525 | 39 | 68 | 57 | 8e−14 | RNA processing |
| 1586 | 2.24692748 | up | 0.000137361 | 0.003112461 | AW705460 | 451 | 78 | 152 | 51 | 9e−28 | transport |
| 1587 | 2.247228524 | up | 0.000093 | 0.002553736 | BE658484 | 306 | 11 | 18 | 61 | 0.85 | |
| 1588 | 2.247383549 | up | 0.0000642 | 0.002135499 | BI970323 | 802 | 88 | 136 | 64 | 2e−46 | proteolysis and peptidolysis |
| 1589 | 2.248376078 | up | 0.002244233 | 0.017358539 | CF808360 | 336 | 34 | 59 | 57 | 5e−12 | |
| 1590 | 2.248996949 | up | 0.000250616 | 0.004407052 | CD396248 | 432 | 17 | 78 | 21 | 1.4 | apoptosis |
| 1591 | 2.250037385 | up | 0.000391361 | 0.005721199 | CF808835 | 283 | 48 | 54 | 88 | 3e−25 | electron transport |
| 1592 | 2.250951469 | up | 0.000235981 | 0.004260065 | CD415130 | 674 | 82 | 158 | 51 | 2e−37 | regulation of transcription |
| 1593 | 2.251050722 | up | 0.00033968 | 0.005277719 | BU547044 | 417 | 17 | 47 | 36 | 5.1 | |
| 1594 | 2.251187854 | up | 0.005179893 | 0.030301163 | BG359691 | 535 | 100 | 174 | 57 | 1e−59 | |
| 1595 | 2.252583139 | up | 0.000186268 | 0.003694813 | BU546336 | 577 | 26 | 83 | 31 | 3e−05 | regulation of transcription\, DNA- |
| | | | | | | | | | | | dependent |
| 1596 | 2.253843386 | up | 0.000183617 | 0.003675665 | BI970354 | 793 | 75 | 134 | 55 | 3e−32 | |
| 1597 | 2.254259724 | up | 0.0000146 | 0.001126538 | CA784614 | 434 | 13 | 34 | 38 | 0.86 | |
| 1598 | 2.25473188 | up | 0.002092097 | 0.016591218 | BU549506 | 344 | 46 | 70 | 65 | 1e−22 | ubiquitin-dependent protein |
| | | | | | | | | | | | catabolism |
| 1599 | 2.254819723 | up | 0.000119393 | 0.002897718 | BE659500 | 750 | 150 | 172 | 87 | 3e−89 | |
| 1600 | 2.254835897 | up | 0.0000525 | 0.001952406 | BI944261 | 536 | 146 | 178 | 82 | 8e−80 | |
| 1601 | 2.255256563 | up | 0.000217356 | 0.004059301 | CF806023 | 652 | 12 | 21 | 57 | 0.38 | electron transport |
| 1602 | 2.255724902 | up | 0.000328263 | 0.00516623 | BI971128 | 667 | 78 | 142 | 54 | 1e−36 | proton transport |
| 1603 | 2.256125241 | up | 0.002190338 | 0.017146172 | AF184278.1 | | | | | | homeodomain-leucine zipper protein |
| | | | | | | | | | | | 57 |
| 1604 | 2.256195324 | up | 0.000019 | 0.001231955 | BE657286 | 829 | 96 | 205 | 46 | 3e−38 | |
| 1605 | 2.256218785 | up | 0.000848528 | 0.009361322 | CD416707 | 748 | 34 | 43 | 79 | 1e−14 | |
| 1606 | 2.256471886 | up | 0.000297245 | 0.004852627 | CD392549 | 659 | 99 | 159 | 62 | 7e−48 | regulation of transcription |
| 1607 | 2.25650455 | up | 0.0000218 | 0.001309763 | BM520173 | 509 | 10 | 27 | 37 | 1.6 | |
| 1608 | 2.256807573 | up | 0.00000609 | 0.000832282 | BM307257 | 211 | 11 | 29 | 37 | 9.5 | |
| 1609 | 2.257330643 | up | 0.000352431 | 0.005371525 | CD416155 | 466 | 47 | 69 | 68 | 3e−22 | |
| 1610 | 2.257631624 | up | 0.00012322 | 0.002948348 | AW351135 | 736 | 72 | 128 | 56 | 6e−38 | |
| 1611 | 2.257744963 | up | 0.003473932 | 0.023045587 | CF807864 | 660 | 145 | 219 | 66 | 1e−82 | proteolysis and peptidolysis |
| 1612 | 2.257877629 | up | 0.0000319 | 0.001527864 | AW350562 | 739 | 93 | 136 | 68 | 3e−49 | |
| 1613 | 2.257940748 | up | 0.000114371 | 0.002833761 | BE804717 | 320 | 8 | 19 | 42 | 7.1 | carbohydrate metabolism |
| 1614 | 2.257955817 | up | 0.0000717 | 0.002248657 | BE803125 | 460 | 56 | 61 | 91 | 3e−30 | regulation of transcription\, DNA- |
| | | | | | | | | | | | dependent |
| 1615 | 2.258268138 | up | 0.000222751 | 0.004112937 | CD412913 | 565 | 63 | 88 | 71 | 1e−31 | |
| 1616 | 2.258935372 | up | 0.002523042 | 0.018747865 | CD416390 | 534 | 32 | 44 | 72 | 3e−14 | |
| 1617 | 2.259174326 | up | 0.000979108 | 0.01021019 | BE021790 | 606 | 113 | 180 | 62 | 9e−60 | proteolysis and peptidolysis |
| 1618 | 2.259609322 | up | 0.001121076 | 0.011155598 | BQ272849 | 492 | 62 | 164 | 37 | 1e−17 | nickel ion transport |
| 1619 | 2.260272784 | up | 0.0000555 | 0.001990649 | BI317172 | | | | | | |
| 1620 | 2.260598019 | up | 0.0000288 | 0.001464287 | BM525641 | 581 | 42 | 61 | 68 | 4e−16 | negative regulation of flower |
| | | | | | | | | | | | development |
| 1621 | 2.261172907 | up | 0.003098296 | 0.021434487 | CD398066 | 410 | 14 | 32 | 43 | 3.8 | |
| 1622 | 2.262486199 | up | 0.000044 | 0.001775674 | BM887283 | 474 | 14 | 26 | 53 | 8.7 | |
| 1623 | 2.262905094 | up | 0.000013 | 0.001078083 | BM887326 | 551 | 119 | 140 | 85 | 5e−67 | |
| 1624 | 2.263490013 | up | 0.0000344 | 0.001564445 | CF805623 | 545 | 104 | 172 | 60 | 1e−54 | DNA metabolism |
| 1625 | 2.26354692 | up | 0.000464515 | 0.006386065 | AI437791 | 412 | 55 | 136 | 40 | 1e−23 | metabolism |
| 1626 | 2.263599957 | up | 0.000494782 | 0.006641564 | CA802910 | | | | | | Isolate x138 magmas-like protein |
| | | | | | | | | | | | mRNA, complete cds; nuclear gene |
| | | | | | | | | | | | for mitochondrial product |
| 1627 | 2.263683762 | up | 0.0000291 | 0.001465395 | BU548009 | 224 | 10 | 23 | 43 | 4.2 | |
| 1628 | 2.263967941 | up | 0.000186137 | 0.003694813 | BG508853 | 188 | 12 | 20 | 60 | 1.9 | |
| 1629 | 2.264775995 | up | 0.000112151 | 0.002806691 | BE440588 | 514 | 93 | 135 | 68 | 4e−57 | |
| 1630 | 2.265245367 | up | 0.000762573 | 0.008774374 | CD414888 | 536 | 127 | 130 | 97 | 8e−70 | similar to Rps15a protein [Gallus |
| | | | | | | | | | | | gallus] |
| 1631 | 2.265490283 | up | 0.000454354 | 0.006308622 | BE658080 | 731 | 86 | 95 | 90 | 2e−44 | protein biosynthesis |
| 1632 | 2.266765442 | up | 0.000107543 | 0.002753653 | BU090090 | 409 | 19 | 57 | 33 | 2.2 | |
| 1633 | 2.26785924 | up | 0.000875319 | 0.009525622 | CD404470 | 664 | 100 | 148 | 67 | 3e−53 | electron transport |
| 1634 | 2.267883616 | up | 0.0000776 | 0.002341624 | BM731664 | 421 | 67 | 79 | 84 | 1e−36 | |
| 1635 | 2.268367546 | up | 0.000279882 | 0.004703793 | AW781111 | 452 | 92 | 130 | 70 | 2e−50 | phytochromobilin biosynthesis |
| 1636 | 2.268558083 | up | 0.0000925 | 0.002551441 | BU763068 | 570 | 116 | 142 | 81 | 2e−67 | protein biosynthesis |
| 1637 | 2.268807389 | up | 0.000348541 | 0.005341571 | CD405025 | 541 | 27 | 49 | 55 | 1e−05 | |
| 1638 | 2.270255792 | up | 0.00121119 | 0.01168224 | BM091606 | 394 | 10 | 23 | 43 | 4.4 | |
| 1639 | 2.270883808 | up | 0.000531524 | 0.006934702 | CD401360 | 225 | 11 | 21 | 52 | 4.2 | |
| 1640 | 2.270900886 | up | 0.003124799 | 0.02155836 | BQ786702 | 609 | 71 | 161 | 44 | 2e−27 | |
| 1641 | 2.270993823 | up | 0.000102167 | 0.002672397 | CD403743 | 644 | 90 | 154 | 58 | 7e−50 | |
| 1642 | 2.271700632 | up | 0.0000554 | 0.001990649 | BI427438 | 426 | 8 | 18 | 44 | 6.9 | regulation of transcription |
| 1643 | 2.271705895 | up | 0.000359545 | 0.005442217 | BG041306 | 365 | 34 | 39 | 87 | 9e−15 | carbohydrate metabolism |
| 1644 | 2.272292622 | up | 0.001141465 | 0.011292661 | BQ081542 | 558 | 124 | 154 | 80 | 9e−72 | acetyl-OcA biosynthesis |
| 1645 | 2.272926453 | up | 0.001053157 | 0.010755751 | CD412731 | 474 | 23 | 26 | 88 | 3e−07 | regulation of translation |
| 1646 | 2.273584688 | up | 0.000244155 | 0.004340089 | BI786994 | 379 | 70 | 119 | 58 | 8e−35 | |
| 1647 | 2.273661647 | up | 0.0000265 | 0.001388554 | BU548170 | 653 | 12 | 36 | 33 | 1.1 | |
| 1648 | 2.274035228 | up | 0.000190245 | 0.003734731 | BU544979 | 587 | 63 | 104 | 60 | 6e−27 | |
| 1649 | 2.274260316 | up | 0.002895126 | 0.020499323 | CF808781 | 460 | 62 | 84 | 73 | 9e−34 | electron transport |
| 1650 | 2.274491142 | up | 0.0000744 | 0.002291616 | BE823100 | 667 | 58 | 186 | 31 | 1e−16 | |
| 1651 | 2.274793208 | up | 0.0000559 | 0.00199765 | BM271038 | 552 | 44 | 84 | 52 | 7e−18 | |
| 1652 | 2.276278265 | up | 0.000208144 | 0.003952102 | CD407062 | 495 | 13 | 29 | 44 | 0.035 | |
| 1653 | 2.276291377 | up | 0.000243991 | 0.004340089 | CD404912 | 710 | 60 | 92 | 65 | 3e−25 | ubiquitin-dependent protein |
| | | | | | | | | | | | catabolism |
| 1654 | 2.277062159 | up | 0.00031687 | 0.005043386 | BU550973 | 653 | 61 | 135 | 45 | 1e−22 | |
| 1655 | 2.277237419 | up | 0.0000694 | 0.0022017 | AI960604 | 535 | 23 | 35 | 65 | 8e−09 | DNA repair |
| 1656 | 2.27732347 | up | 0.000114589 | 0.002833761 | BG882314 | 455 | 27 | 30 | 90 | 7e−12 | electron transport |
| 1657 | 2.277915312 | up | 0.000120056 | 0.002902004 | BU546781 | 612 | 73 | 103 | 70 | 7e−37 | |
| 1658 | 2.278987918 | up | 0.000384921 | 0.005649006 | BI970187 | 772 | 54 | 127 | 42 | 3e−18 | |
| 1659 | 2.279880213 | up | 0.000423943 | 0.006010626 | BI471672 | 448 | 55 | 175 | 31 | 1e−11 | |
| 1660 | 2.280360743 | up | 0.000461589 | 0.006364445 | CA819148 | | | | | | |
| 1661 | 2.280739875 | up | 0.0000359 | 0.001603515 | BF066536 | 585 | 126 | 195 | 64 | 1e−71 | thiamin metabolism |
| 1662 | 2.280821856 | up | 0.000846438 | 0.009342113 | CD393012 | 675 | 46 | 71 | 64 | 6e−21 | |
| 1663 | 2.281350084 | up | 0.002554582 | 0.018914973 | BG362696 | 514 | 59 | 105 | 56 | 3e−32 | protein biosynthesis |
| 1664 | 2.281484335 | up | 0.0000183 | 0.00122288 | CD414059 | 586 | 26 | 47 | 55 | 2e−06 | |
| 1665 | 2.282192798 | up | 0.0000145 | 0.001125207 | BU548826 | 654 | 16 | 59 | 27 | 2.5 | carbohydrate metabolism |
| 1666 | 2.283405489 | up | 0.0000655 | 0.002157866 | CD400086 | 734 | 89 | 97 | 91 | 7e−48 | protein biosynthesis |
| 1667 | 2.283699325 | up | 0.000260294 | 0.004496644 | CD412243 | 587 | 115 | 162 | 70 | 2e−64 | |
| 1668 | 2.284250445 | up | 0.000264732 | 0.004540648 | CD392549 | 659 | 99 | 159 | 62 | 7e−48 | regulation of transcription |
| 1669 | 2.284482245 | up | 0.0000162 | 0.001154209 | BM567880 | 326 | 14 | 36 | 38 | 3.2 | |
| 1670 | 2.284498393 | up | 0.00184256 | 0.015238798 | BU761288 | 309 | 20 | 26 | 76 | 7e−05 | |
| 1671 | 2.284766481 | up | 0.0000576 | 0.002024457 | BU548244 | 648 | 91 | 169 | 53 | 1e−44 | metabolism |
| 1672 | 2.286043098 | up | 0.0000835 | 0.0024421 | AW185955 | 524 | 59 | 110 | 53 | 1e−26 | protein biosynthesis |
| 1673 | 2.286877792 | up | 0.00232441 | 0.017738797 | BF595424 | 443 | 17 | 51 | 33 | 2.0 | proteolysis and peptidolysis |
| 1674 | 2.288582486 | up | 0.000819771 | 0.009136253 | CD392224 | 713 | 167 | 192 | 86 | 4e−94 | |
| 1675 | 2.2895088 | up | 0.0023055 | 0.017647353 | CF809057 | 788 | 152 | 210 | 72 | 2e−94 | |
| 1676 | 2.289617433 | up | 0.00633637 | 0.034793061 | BI943943 | 320 | 82 | 101 | 81 | 1e−38 | DNA methylation |
| 1677 | 2.289726022 | up | 0.002184132 | 0.017120452 | CD412189 | 607 | 65 | 79 | 82 | 2e−30 | |
| 1678 | 2.290385119 | up | 0.000218455 | 0.004072519 | AI442972 | 588 | 120 | 138 | 86 | 4e−61 | two-component signal transduction |
| | | | | | | | | | | | system (phosphorelay) |
| 1679 | 2.290392991 | up | 0.0000632 | 0.002118327 | CD396871 | 658 | 13 | 40 | 32 | 4.2 | |
| 1680 | 2.290595281 | up | 0.000673571 | 0.008066121 | BE475047 | 503 | 127 | 167 | 76 | 1e−76 | |
| 1681 | 2.292403311 | up | 0.000510992 | 0.006772537 | BE821347 | 583 | 86 | 116 | 74 | 2e−44 | Isolate x138 magmas-like protein |
| | | | | | | | | | | | mRNA, complete cds; nuclear gene |
| | | | | | | | | | | | for mitochondrial product |
| 1682 | 2.29248283 | up | 0.000538898 | 0.006987285 | CK606334 | 717 | 118 | 208 | 56 | 1e−65 | Glutathione S-transferase GST 8, |
| | | | | | | | | | | | transport |
| 1683 | 2.293107775 | up | 0.00046275 | 0.006373439 | BM095114 | 471 | 121 | 156 | 77 | 5e−67 | |
| 1684 | 2.293723688 | up | 0.0000155 | 0.001135545 | CD398725 | 693 | 116 | 143 | 81 | 3e−64 | |
| 1685 | 2.297839951 | up | 0.0000151 | 0.001128338 | CD403737 | | | | | | |
| 1686 | 2.298386774 | up | 0.0000903 | 0.002528493 | CD395607 | 442 | 19 | 29 | 65 | 6e−07 | |
| 1687 | 2.298739169 | up | 0.00000955 | 0.000930763 | BQ785421 | 591 | 159 | 190 | 83 | 1e−90 | |
| 1688 | 2.299268994 | up | 0.0000613 | 0.002087031 | BG237049 | | | | | | |
| 1689 | 2.299616725 | up | 0.000340628 | 0.005277719 | BI893280 | 576 | 91 | 145 | 62 | 4e−47 | |
| 1690 | 2.300261952 | up | 0.008423116 | 0.041984062 | BE348171 | 543 | 136 | 182 | 74 | 1e−76 | protein biosynthesis |
| 1691 | 2.301813714 | up | 0.000119486 | 0.002897718 | AW734066 | 343 | 18 | 67 | 26 | 0.85 | |
| 1692 | 2.302137117 | up | 0.0000464 | 0.001810721 | BM885005 | 413 | 13 | 34 | 38 | 0.20 | |
| 1693 | 2.302627321 | up | 0.000478526 | 0.006502633 | CA782862 | 569 | 17 | 21 | 80 | 0.035 | |
| 1694 | 2.303424576 | up | 0.00018313 | 0.003669828 | AI794798 | 478 | 17 | 49 | 34 | 8.8 | malate metabolism |
| 1695 | 2.303840217 | up | 0.000368675 | 0.005500733 | CD396940 | 719 | 116 | 151 | 76 | 1e−59 | |
| 1696 | 2.303877848 | up | 0.001105341 | 0.011055873 | CD416074 | | | | | | |
| 1697 | 2.304421367 | up | 0.00014009 | 0.003145882 | BQ628119 | 235 | 12 | 40 | 30 | 1.4 | |
| 1698 | 2.304831435 | up | 0.0000423 | 0.001755385 | CA801873 | 662 | 130 | 156 | 83 | 3e−69 | |
| 1699 | 2.305388852 | up | 0.000634715 | 0.007817744 | CA852636 | 402 | 24 | 39 | 61 | 4e−04 | protein biosynthesis |
| 1700 | 2.305842247 | up | 0.000254122 | 0.004437577 | AW310095 | 648 | 87 | 144 | 60 | 6e−47 | |
| 1701 | 2.307163567 | up | 0.000261269 | 0.004506983 | AW705955 | 441 | 83 | 150 | 55 | 6e−43 | |
| 1702 | 2.307287276 | up | 0.000247832 | 0.004383337 | BU927254 | 538 | 20 | 33 | 60 | 1e−05 | |
| 1703 | 2.307998998 | up | 0.0000913 | 0.002541909 | BM732954 | 460 | 8 | 23 | 34 | 6.3 | |
| 1704 | 2.309844254 | up | 0.003003664 | 0.02101573 | BG362818 | | | | | | polyamine-transporting ATPase |
| | | | | | | | | | | | activity |
| 1705 | 2.3099965 | up | 0.000767653 | 0.0088032 | BU544443 | 440 | 15 | 35 | 42 | 2.6 | |
| 1706 | 2.31061007 | up | 0.000545259 | 0.007044501 | CD392100 | 615 | 124 | 126 | 98 | 6e−65 | nucleosome assembly |
| 1707 | 2.310633168 | up | 0.0000858 | 0.002466308 | BG406170 | 370 | 88 | 124 | 70 | 6e−43 | |
| 1708 | 2.312165403 | up | 0.000880722 | 0.009568269 | CF808470 | 156 | 35 | 52 | 67 | 3e−15 | |
| 1709 | 2.312516252 | up | 0.000254788 | 0.004443909 | AW309567 | 457 | 15 | 42 | 35 | 0.56 | protein complex assembly |
| 1710 | 2.313546318 | up | 0.0000251 | 0.001368059 | CD397651 | 616 | 43 | 67 | 64 | 4e−17 | |
| 1711 | 2.31404791 | up | 0.000152635 | 0.003302322 | AW756517 | 573 | 137 | 162 | 84 | 7e−72 | |
| 1712 | 2.31443658 | up | 0.000822787 | 0.0091536 | CD401298 | 453 | 32 | 52 | 61 | 1e−14 | electron transport |
| 1713 | 2.314784471 | up | 0.000190761 | 0.003737135 | AI494968 | 384 | 51 | 114 | 44 | 8e−17 | transport |
| 1714 | 2.315935454 | up | 0.000717833 | 0.008440317 | BE823347 | 602 | 88 | 110 | 80 | 4e−46 | |
| 1715 | 2.317023651 | up | 0.00019721 | 0.00381185 | BI970589 | 700 | 100 | 172 | 58 | 2e−52 | regulation of transcription\, DNA- |
| | | | | | | | | | | | dependent |
| 1716 | 2.317214783 | up | 0.0000191 | 0.001231955 | BE020696 | 421 | 102 | 139 | 73 | 3e−52 | tRNA processing |
| 1717 | 2.317472712 | up | 0.000181537 | 0.003658009 | BM307866 | 596 | 96 | 163 | 58 | 2e−51 | |
| 1718 | 2.317957266 | up | 0.000243917 | 0.004340089 | AW119881 | 371 | 10 | 24 | 41 | 2.9 | electron transport |
| 1719 | 2.319098671 | up | 0.001498117 | 0.013330269 | AW597363 | 423 | 14 | 35 | 40 | 1.8 | apoptosis |
| 1720 | 2.320598303 | up | 0.007723561 | 0.039553882 | BU548014 | 511 | 13 | 41 | 31 | 2.0 | regulation of transcription |
| 1721 | 2.320640071 | up | 0.000194966 | 0.003785091 | CD398581 | 553 | 83 | 99 | 83 | 8e−41 | |
| 1722 | 2.320844913 | up | 0.000797788 | 0.008981739 | CD414845 | 655 | 60 | 93 | 64 | 2e−34 | metabolism |
| 1723 | 2.321385393 | up | 0.000278423 | 0.004687827 | BI700789 | 562 | 85 | 104 | 81 | 1e−47 | |
| 1724 | 2.322401016 | up | 0.000303388 | 0.004912464 | CF807754 | 570 | 86 | 167 | 51 | 1e−44 | |
| 1725 | 2.322858936 | up | 0.001799454 | 0.015044969 | BU082681 | 587 | 101 | 209 | 48 | 5e−45 | |
| 1726 | 2.322996256 | up | 0.002404862 | 0.018148186 | BE657171 | 562 | 50 | 64 | 78 | 6e−23 | regulation of transcription\, DNA- |
| | | | | | | | | | | | dependent |
| 1727 | 2.323139125 | up | 0.00113437 | 0.011238106 | BI470399 | 559 | 58 | 185 | 31 | 2e−19 | metabolism |
| 1728 | 2.323360664 | up | 0.002834731 | 0.020247924 | CD403269 | 711 | 151 | 184 | 82 | 5e−84 | biological_process unknown |
| 1729 | 2.323631918 | up | 0.000216914 | 0.004056766 | BM086011 | 529 | 108 | 159 | 67 | 4e−60 | |
| 1730 | 2.324107135 | up | 0.0000186 | 0.00122629 | BU548236 | 553 | 23 | 28 | 82 | 4e−07 | thiamin metabolism |
| 1731 | 2.324834975 | up | 0.000545545 | 0.007044501 | CK606540 | 724 | 94 | 113 | 83 | 3e−49 | protein biosynthesis |
| 1732 | 2.325073263 | up | 0.000562251 | 0.007183592 | BU550882 | 452 | 70 | 109 | 64 | 1e−36 | |
| 1733 | 2.325266896 | up | 0.00101651 | 0.010511836 | CD418407 | 387 | 13 | 15 | 86 | 0.026 | |
| 1734 | 2.326683314 | up | 0.000110663 | 0.002790944 | CD392616 | 688 | 101 | 120 | 84 | 9e−54 | DNA metabolism |
| 1735 | 2.32684301 | up | 0.005578668 | 0.031811471 | AW734373 | 554 | 49 | 175 | 28 | 3e−13 | |
| 1736 | 2.327758498 | up | 0.0000464 | 0.001810721 | BU578612 | 565 | 103 | 134 | 76 | 1e−54 | tRNA aminoacylation for protein |
| | | | | | | | | | | | translation |
| 1737 | 2.328131802 | up | 0.001506405 | 0.013369968 | BM270810 | | | | | | |
| 1738 | 2.328896561 | up | 0.000691748 | 0.008233993 | AI437803 | 444 | 55 | 118 | 46 | 3e−18 | |
| 1739 | 2.329429963 | up | 0.0000103 | 0.000963549 | BF426017 | 422 | 68 | 79 | 86 | 2e−34 | |
| 1740 | 2.329678453 | up | 0.0000753 | 0.002305548 | BG406414 | 349 | 15 | 50 | 30 | 0.13 | |
| 1741 | 2.329885344 | up | 0.004581791 | 0.027859897 | BE659769 | 209 | 15 | 49 | 30 | 0.86 | DNA transposition |
| 1742 | 2.330350816 | up | 0.0000231 | 0.001328603 | BM177712 | 541 | 48 | 103 | 46 | 4e−22 | |
| 1743 | 2.330732303 | up | 0.004679619 | 0.028267093 | CA801968 | 450 | 59 | 100 | 59 | 4e−27 | |
| 1744 | 2.331665183 | up | 0.002514636 | 0.018708265 | AW200959 | 407 | 75 | 102 | 73 | 5e−40 | tRNA aminoacylation for protein |
| | | | | | | | | | | | translation |
| 1745 | 2.332425003 | up | 0.0000538 | 0.001973239 | BE607683 | 437 | 122 | 145 | 84 | 7e−67 | |
| 1746 | 2.332434105 | up | 0.001838786 | 0.015223166 | AW756576 | 557 | 117 | 185 | 63 | 3e−64 | |
| 1747 | 2.332672032 | up | 0.000106653 | 0.00273398 | BF324108 | 464 | 37 | 42 | 88 | 4e−15 | |
| 1748 | 2.333258651 | up | 0.0003734 | 0.005553607 | AY157740.1 | | | | | | nucleoside diphosphate kinase |
| 1749 | 2.334549178 | up | 0.000081 | 0.002407641 | BU550315 | 624 | 86 | 157 | 54 | 1e−47 | metabolism |
| 1750 | 2.335040212 | up | 0.001807838 | 0.015080956 | CD397742 | 569 | 38 | 78 | 48 | 6e−13 | |
| 1751 | 2.335702879 | up | 0.000444234 | 0.006204759 | CD416430 | 261 | 12 | 38 | 31 | 7.2 | protein amino acid phosphorylation |
| 1752 | 2.336686511 | up | 0.000886799 | 0.009603513 | AI856445 | 497 | 18 | 54 | 33 | 2.0 | embryonic development (sensu |
| | | | | | | | | | | | Magnoliophyta) |
| 1753 | 2.337054412 | up | 0.000341654 | 0.005281591 | BU090990 | 427 | 22 | 36 | 61 | 9e−08 | |
| 1754 | 2.337437494 | up | 0.002136618 | 0.016880225 | CA802642 | 301 | 10 | 13 | 76 | 1.1 | electron transport |
| 1755 | 2.337520347 | up | 0.000143239 | 0.003181853 | AI431092 | 487 | 50 | 71 | 70 | 1e−22 | nickel ion transport |
| 1756 | 2.337543759 | up | 0.00031538 | 0.00503724 | BE057309 | 148 | 11 | 31 | 35 | 1.4 | cation transport |
| 1757 | 2.337605039 | up | 0.0000133 | 0.001087368 | CA782979 | 729 | 75 | 171 | 43 | 3e−26 | cell cycle |
| 1758 | 2.337993474 | up | 0.0001857 | 0.003694813 | BI967250 | 761 | 97 | 168 | 57 | 8e−49 | proteolysis and peptidolysis |
| 1759 | 2.338386339 | up | 0.0000476 | 0.001830505 | BQ094150 | 504 | 46 | 75 | 61 | 6e−19 | |
| 1760 | 2.338484409 | up | 0.000167849 | 0.00348617 | BI424495 | 405 | 49 | 89 | 55 | 3e−22 | |
| 1761 | 2.33875277 | up | 0.000306541 | 0.00494436 | BU091712 | 382 | 16 | 48 | 33 | 0.009 | regulation of transcription\, DNA- |
| | | | | | | | | | | | dependent |
| 1762 | 2.339665796 | up | 0.000163297 | 0.003427588 | BF067737 | 391 | 12 | 16 | 75 | 1.9 | |
| 1763 | 2.341118699 | up | 0.0000946 | 0.002566638 | BQ298724 | 278 | 11 | 16 | 68 | 1.9 | transport |
| 1764 | 2.341712271 | up | 0.000913541 | 0.009770243 | BU547165 | 626 | 61 | 76 | 80 | 3e−29 | protein biosynthesis |
| 1765 | 2.34238086 | up | 0.00170114 | 0.014481366 | BG044600 | 255 | 68 | 85 | 80 | 4e−34 | small GTPase mediated signal |
| | | | | | | | | | | | transduction |
| 1766 | 2.343153074 | up | 0.000121815 | 0.002929629 | BE822326 | 781 | 101 | 138 | 73 | 2e−52 | protein biosynthesis |
| 1767 | 2.34330526 | up | 0.001028089 | 0.010603379 | AW781988 | 547 | 110 | 182 | 60 | 2e−61 | nuclear membrane fusion |
| 1768 | 2.343407185 | up | 0.0000772 | 0.002341624 | BQ611546 | 322 | 12 | 21 | 57 | 0.64 | DNA recombination |
| 1769 | 2.34364951 | up | 0.0000683 | 0.002184143 | BM731770 | 462 | 40 | 44 | 90 | 4e−17 | tricarboxylic acid cycle |
| 1770 | 2.343909636 | up | 0.000115787 | 0.002848619 | CD408814 | 638 | 70 | 107 | 65 | 2e−33 | |
| 1771 | 2.345019057 | up | 0.000242498 | 0.004324949 | BM085538 | | | | | | |
| 1772 | 2.345357744 | up | 0.007783464 | 0.039745753 | CA784199 | 700 | 168 | 216 | 77 | 6e−94 | glycolysis |
| 1773 | 2.345660963 | up | 0.0000839 | 0.002448136 | BI471267 | 421 | 56 | 128 | 43 | 1e−22 | |
| 1774 | 2.345675827 | up | 0.000507527 | 0.006740857 | CD408801 | 289 | 23 | 80 | 28 | 0.012 | protein amino acid phosphorylation |
| 1775 | 2.347436345 | up | 0.000118518 | 0.002885407 | BE352683 | 496 | 104 | 153 | 67 | 2e−55 | electron transport |
| 1776 | 2.347555114 | up | 0.000252543 | 0.004420006 | CD405051 | 666 | 64 | 92 | 69 | 6e−31 | |
| 1777 | 2.347973176 | up | 0.000225411 | 0.004139945 | BQ610144 | 303 | 15 | 30 | 50 | 0.50 | |
| 1778 | 2.348003631 | up | 0.000819381 | 0.009135628 | BE820446 | 592 | 40 | 75 | 53 | 9e−17 | regulation of transcription\, DNA- |
| | | | | | | | | | | | dependent |
| 1779 | 2.349014495 | up | 0.000054 | 0.001976072 | CD408814 | 638 | 70 | 107 | 65 | 2e−33 | |
| 1780 | 2.349110379 | up | 0.000087 | 0.00247839 | BI471175 | 581 | 116 | 192 | 60 | 6e−67 | |
| 1781 | 2.34917076 | up | 0.001445259 | 0.01300662 | BM187652 | 587 | 35 | 81 | 43 | 5e−13 | |
| 1782 | 2.350126917 | up | 0.0000742 | 0.00228806 | AW310367 | 435 | 21 | 64 | 32 | 0.001 | response to light |
| 1783 | 2.350908899 | up | 0.00015434 | 0.003322585 | BU083650 | 254 | 14 | 21 | 66 | 1.9 | transcription |
| 1784 | 2.351568104 | up | 0.000782137 | 0.008904149 | BQ627846 | 439 | 18 | 61 | 29 | 1.5 | |
| 1785 | 2.352628912 | up | 0.0005226 | 0.006866143 | BG044578 | 682 | 173 | 223 | 77 | 2e−98 | amino acid metabolism |
| 1786 | 2.354348216 | up | 0.0000532 | 0.00195852 | AF475939.1 | | | | | | elongation factor 1-gamma |
| 1787 | 2.355554215 | up | 0.000557072 | 0.007139256 | BQ610084 | 533 | 75 | 95 | 78 | 6e−41 | |
| 1788 | 2.355717599 | up | 0.0000261 | 0.001380871 | BI427045 | 421 | 19 | 33 | 57 | 0.001 | nucleosome assembly |
| 1789 | 2.356830319 | up | 0.0000649 | 0.002146014 | BE821479 | 605 | 84 | 167 | 50 | 3e−48 | apoptosis |
| 1790 | 2.358341454 | up | 0.0000867 | 0.002474922 | CD414598 | 683 | 76 | 89 | 85 | 1e−39 | |
| 1791 | 2.359686093 | up | 0.0000431 | 0.00176847 | CA782622 | 748 | 92 | 167 | 55 | 3e−41 | |
| 1792 | 2.359704367 | up | 0.0000298 | 0.00147316 | BU548798 | 692 | 101 | 167 | 60 | 3e−53 | protein biosynthesis |
| 1793 | 2.361331332 | up | 0.00018824 | 0.003712885 | BM885917 | 591 | 143 | 199 | 71 | 5e−77 | DNA metabolism |
| 1794 | 2.361861091 | up | 0.000209111 | 0.003962467 | BE211336 | 611 | 80 | 168 | 47 | 1e−38 | intracellular protein transporl |
| 1795 | 2.362006041 | up | 0.000177651 | 0.00361419 | BE608520 | 619 | 154 | 173 | 89 | 2e−86 | Vacuolar processing enzyme 2 |
| | | | | | | | | | | | (VPE2), protein catabolism |
| 1796 | 2.362076199 | up | 0.0000131 | 0.001083311 | BU544080 | 455 | 18 | 51 | 35 | 0.56 | |
| 1797 | 2.362077798 | up | 0.000163488 | 0.003427788 | CD392529 | 642 | 68 | 110 | 61 | 2e−33 | |
| 1798 | 2.362278735 | up | 0.001178479 | 0.011516578 | BM891443 | 555 | 26 | 37 | 70 | 9e−08 | ATP/ADP exchange |
| 1799 | 2.362473036 | up | 0.000238904 | 0.004290802 | BF068015 | 479 | 48 | 80 | 60 | 8e−23 | |
| 1800 | 2.363889977 | up | 0.001616675 | 0.013988873 | BM890751 | 411 | 42 | 50 | 84 | 4e−19 | |
| 1801 | 2.363920105 | up | 0.001300905 | 0.012238819 | AF255338.1 | | | | | | 20S proteasome subunit |
| 1802 | 2.365038062 | up | 0.000176061 | 0.003591302 | BI969464 | 719 | 47 | 78 | 60 | 9e−18 | |
| 1803 | 2.365084128 | up | 0.010716019 | 0.049308887 | AW306163 | 394 | 48 | 58 | 82 | 5e−23 | |
| 1804 | 2.365320478 | up | 0.000349308 | 0.005347529 | BU762938 | 601 | 99 | 158 | 62 | 8e−53 | porphyrin biosynthesis |
| 1805 | 2.365568581 | up | 0.001349309 | 0.012488408 | CD397370 | 642 | 98 | 140 | 70 | 3e−50 | |
| 1806 | 2.365756404 | up | 0.0000428 | 0.001762775 | AW309274 | 618 | 103 | 121 | 85 | 1e−55 | sex determination |
| 1807 | 2.366456024 | up | 0.000323384 | 0.005115125 | CA819655 | 358 | 31 | 40 | 77 | 7e−14 | transport |
| 1808 | 2.369102667 | up | 0.000152681 | 0.003302322 | CD397282 | 592 | 95 | 105 | 90 | 3e−54 | protein biosynthesis |
| 1809 | 2.369455606 | up | 0.0000355 | 0.001589423 | BU964854 | 583 | 96 | 159 | 60 | 4e−47 | |
| 1810 | 2.369507905 | up | 0.001028923 | 0.010606169 | CF808786 | 578 | 103 | 145 | 71 | 1e−59 | |
| 1811 | 2.36987189 | up | 0.0000683 | 0.002184143 | BI968821 | 637 | 35 | 80 | 43 | 1e−11 | |
| 1812 | 2.370419513 | up | 0.0000919 | 0.00254498 | BI972147 | 550 | 85 | 168 | 50 | 8e−36 | |
| 1813 | 2.370674292 | up | 0.000042 | 0.001750979 | CD411279 | 673 | 90 | 123 | 73 | 3e−51 | |
| 1814 | 2.371101363 | up | 0.000296824 | 0.00485001 | CD400543 | 652 | 106 | 136 | 77 | 1e−59 | protein biosynthesis |
| 1815 | 2.372326063 | up | 0.000265368 | 0.004547411 | CD391812 | 603 | 65 | 69 | 94 | 3e−32 | protein biosynthesis |
| 1816 | 2.372388635 | up | 0.000157513 | 0.003347279 | BM085101 | 464 | 61 | 68 | 89 | 3e−33 | electron transport |
| 1817 | 2.372460178 | up | 0.0000503 | 0.001900471 | BU546631 | 608 | 72 | 136 | 52 | 6e−37 | protein biosynthesis |
| 1818 | 2.372496809 | up | 0.0000305 | 0.00148682 | BU760640 | 380 | 15 | 46 | 32 | 1.1 | |
| 1819 | 2.372532313 | up | 0.000393867 | 0.005742215 | BU760708 | 495 | 18 | 41 | 43 | 0.001 | |
| 1820 | 2.372675512 | up | 0.0000178 | 0.001214142 | CD394299 | 481 | 51 | 62 | 82 | 5e−24 | |
| 1821 | 2.37384978 | up | 0.0000934 | 0.002553736 | BG508850 | 479 | 49 | 75 | 65 | 5e−25 | G-protein coupled receptor protein |
| | | | | | | | | | | | signaling pathway |
| 1822 | 2.374978948 | up | 0.0000476 | 0.001830505 | CD397332 | 569 | 81 | 116 | 69 | 5e−44 | DNA metabolism |
| 1823 | 2.376054532 | up | 0.0000501 | 0.001898772 | CA820571 | 423 | 15 | 39 | 38 | 0.055 | regulation of transcription\, DNA- |
| | | | | | | | | | | | dependent |
| 1824 | 2.37642516 | up | 0.006247289 | 0.034440205 | BE821093 | 725 | 18 | 41 | 43 | 0.20 | protein amino acid phosphorylation |
| 1825 | 2.376462558 | up | 0.000295584 | 0.00483396 | BE473742 | 674 | 165 | 220 | 75 | 1e−92 | |
| 1826 | 2.376482922 | up | 0.000186384 | 0.003694813 | CD406330 | 447 | 48 | 71 | 67 | 2e−23 | |
| 1827 | 2.376666288 | up | 0.0000714 | 0.002244606 | AI965552 | 421 | 38 | 48 | 79 | 3e−18 | |
| 1828 | 2.376859875 | up | 0.000465574 | 0.00638895 | CD407874 | 700 | 134 | 154 | 87 | 7e−73 | adenine salvage pathway |
| 1829 | 2.377090575 | up | 0.0000746 | 0.002293856 | BG651951 | 489 | 22 | 40 | 55 | 1e−09 | protein-mitochondrial targeting |
| 1830 | 2.378013307 | up | 0.0000231 | 0.001328603 | AW279008 | 452 | 99 | 148 | 66 | 6e−54 | |
| 1831 | 2.378721692 | up | 0.0000694 | 0.0022017 | AI748705 | 520 | 119 | 173 | 68 | 3e−70 | threonine catabolism |
| 1832 | 2.378928067 | up | 0.002181213 | 0.017117299 | BU084188 | 373 | 15 | 37 | 40 | 0.12 | tricarboxylic acid cycle |
| 1833 | 2.379048821 | up | 0.003293847 | 0.022339478 | CF807221 | 461 | 80 | 151 | 52 | 2e−45 | protein catabolism |
| 1834 | 2.379441839 | up | 0.000093 | 0.002553736 | BM139593 | 259 | 10 | 26 | 38 | 9.4 | cation transport |
| 1835 | 2.379697971 | up | 0.000532029 | 0.006936475 | BE823824 | 637 | 108 | 167 | 64 | 7e−64 | histone deacetylation |
| 1836 | 2.380700946 | up | 0.0000489 | 0.001870412 | BM307863 | 581 | 86 | 196 | 43 | 3e−36 | |
| 1837 | 2.38220102 | up | 0.0000237 | 0.00133979 | BE346175 | 494 | 50 | 120 | 41 | 2e−17 | nuclear membrane fusion |
| 1838 | 2.382407385 | up | 0.010720946 | 0.049308887 | AW395344 | 427 | 10 | 19 | 52 | 1.8 | two-component signal transduction |
| | | | | | | | | | | | system (phosphorelay) |
| 1839 | 2.382707933 | up | 0.001615542 | 0.013985643 | CD414873 | 509 | 15 | 36 | 41 | 2.7 | metabolism |
| 1840 | 2.384260775 | up | 0.000103498 | 0.002690367 | BE329777 | 463 | 40 | 99 | 40 | 2e−15 | |
| 1841 | 2.385005975 | up | 0.000486985 | 0.006585943 | CD397034 | 584 | 59 | 80 | 73 | 2e−33 | cell cycle |
| 1842 | 2.385439878 | up | 0.0000409 | 0.001731672 | BM527862 | 442 | 12 | 49 | 24 | 1.2 | |
| 1843 | 2.385898927 | up | 0.0000861 | 0.002468444 | CD395174 | 374 | 13 | 40 | 32 | 0.58 | |
| 1844 | 2.386082149 | up | 0.0000363 | 0.001613718 | BI320490 | 549 | 145 | 182 | 79 | 5e−82 | galactose metabolism |
| 1845 | 2.387469255 | up | 0.00000398 | 0.000767688 | BU550127 | 615 | 123 | 175 | 70 | 2e−75 | |
| 1846 | 2.387483077 | up | 0.004328942 | 0.026794278 | AW596467 | 493 | 129 | 163 | 79 | 3e−76 | carbohydrate metabolism |
| 1847 | 2.387514229 | up | 0.000895076 | 0.009660659 | AW570594 | 609 | 178 | 201 | 88 | 3e−99 | BiP isoform C |
| 1848 | 2.387994103 | up | 0.000357614 | 0.0054261 | BU760658 | 306 | 13 | 50 | 26 | 1.9 | |
| 1849 | 2.389424098 | up | 0.00069416 | 0.008254865 | BU927530 | 609 | 65 | 148 | 43 | 3e−31 | |
| 1850 | 2.390688682 | up | 0.000152763 | 0.003302322 | BU549068 | 518 | 112 | 138 | 81 | 4e−53 | microtubule-based movement |
| 1851 | 2.392206 | up | 0.001656386 | 0.014227751 | AF034572.1 | | | | | | Proteasome IOTA subunit |
| 1852 | 2.39268264 | up | 0.0000166 | 0.00116924 | BU546978 | 665 | 70 | 132 | 53 | 6e−32 | |
| 1853 | 2.394923693 | up | 0.001481249 | 0.013230992 | BM954903 | 435 | 16 | 28 | 57 | 0.23 | |
| 1854 | 2.395065069 | up | 0.000042 | 0.001750979 | AF271071.1 | | | | | | biotin carboxyl carrier protein subunit |
| | | | | | | | | | | | precursor |
| 1855 | 2.39558524 | up | 0.0000333 | 0.001543897 | BI315806 | 429 | 65 | 86 | 75 | 3e−31 | |
| 1856 | 2.395758843 | up | 0.004257645 | 0.026444364 | CA852105 | 692 | 41 | 77 | 53 | 1e−15 | proton transport |
| 1857 | 2.396062467 | up | 0.0000146 | 0.001125207 | CA852887 | 483 | 9 | 18 | 50 | 5.3 | electron transport |
| 1858 | 2.396633248 | up | 0.000376081 | 0.005575844 | BM522772 | 461 | 9 | 22 | 40 | 6.4 | |
| 1859 | 2.396815798 | up | 0.0000959 | 0.002588247 | BG551024 | 476 | 139 | 159 | 87 | 2e−73 | nicotinamide adenine dinucleotide |
| | | | | | | | | | | | biosynthesis |
| 1860 | 2.3974983 | up | 0.0000697 | 0.002203792 | BE823343 | 709 | 34 | 66 | 51 | 4e−12 | anion transport |
| 1861 | 2.397612757 | up | 0.001301669 | 0.012240241 | CD400844 | 574 | 14 | 39 | 35 | 4.4 | |
| 1862 | 2.398839515 | up | 0.003058153 | 0.021254505 | BQ612558 | 404 | 25 | 77 | 32 | 0.19 | cell death |
| 1863 | 2.398970456 | up | 0.000362168 | 0.005457831 | AW832353 | 499 | 126 | 155 | 81 | 2e−71 | protein folding |
| 1864 | 2.399070613 | up | 0.00015761 | 0.00334746 | CD400228 | 493 | 13 | 34 | 38 | 1.1 | |
| 1865 | 2.400581701 | up | 0.002161479 | 0.017044419 | BM731397 | 575 | 42 | 65 | 64 | 1e−19 | |
| 1866 | 2.400960022 | up | 0.00012288 | 0.002947703 | CA953106 | 565 | 12 | 26 | 46 | 1.5 | electron transport |
| 1867 | 2.40111828 | up | 0.000331508 | 0.005204234 | CD414299 | 631 | 56 | 71 | 78 | 5e−24 | pyruvate kinase, glycolysis |
| 1868 | 2.401128815 | up | 0.000156993 | 0.003347279 | AW307459 | 276 | 14 | 42 | 33 | 4.2 | |
| 1869 | 2.401212211 | up | 0.0000422 | 0.001755005 | CD404469 | 652 | 54 | 94 | 57 | 1e−25 | |
| 1870 | 2.402332812 | up | 0.0000433 | 0.001770252 | BQ081508 | 440 | 37 | 61 | 60 | 1e−15 | |
| 1871 | 2.402481965 | up | 0.0000718 | 0.002248657 | BI787878 | 497 | 51 | 67 | 76 | 1e−25 | |
| 1872 | 2.402727961 | up | 0.002283926 | 0.017546483 | BM568376 | 570 | 97 | 105 | 92 | 2e−54 | |
| 1873 | 2.402921528 | up | 0.000505788 | 0.00672965 | BQ298270 | 325 | 17 | 54 | 31 | 1.4 | |
| 1874 | 2.406188463 | up | 0.0000306 | 0.00148682 | BQ610726 | 607 | 121 | 179 | 67 | 3e−70 | proteolysis and peptidolysis |
| 1875 | 2.406830759 | up | 0.002209465 | 0.017220643 | AW706349 | | | | | | |
| 1876 | 2.407407057 | up | 0.000899319 | 0.009683106 | CD390751 | 611 | 15 | 62 | 24 | 2.2 | beta-1\, 3 glucan biosynthesis |
| 1877 | 2.407934087 | up | 0.0000778 | 0.002341624 | BE805276 | 612 | 139 | 188 | 73 | 2e−75 | |
| 1878 | 2.40815815 | up | 0.000255898 | 0.004454124 | BI786109 | 545 | 15 | 22 | 68 | 0.011 | |
| 1879 | 2.408517722 | up | 0.0000885 | 0.002505747 | CD390538 | 466 | 14 | 26 | 53 | 0.20 | regulation of flower development |
| 1880 | 2.409256739 | up | 0.0000378 | 0.001657189 | BI972851 | 258 | 32 | 38 | 84 | 4e−11 | ossification |
| 1881 | 2.410144062 | up | 0.0000456 | 0.001803554 | BM306984 | 413 | 21 | 29 | 72 | 2e−06 | DNA metabolism |
| 1882 | 2.410353807 | up | 0.0000669 | 0.002172462 | BG653341 | 346 | 37 | 58 | 63 | 2e−16 | |
| 1883 | 2.410407975 | up | 0.000119629 | 0.002899314 | BI971042 | 645 | 74 | 93 | 79 | 5e−40 | ATP metabolism |
| 1884 | 2.410698003 | up | 0.000333984 | 0.005229615 | CD404099 | 691 | 83 | 99 | 83 | 3e−43 | glycolysis |
| 1885 | 2.411566044 | up | 0.00362737 | 0.023745193 | BU548256 | 626 | 97 | 129 | 75 | 6e−59 | lipid catabolism |
| 1886 | 2.412431185 | up | 0.000780687 | 0.008897094 | BI468832 | 470 | 39 | 74 | 52 | 6e−17 | purine ribonucleoside |
| | | | | | | | | | | | monophosphate biosynthesis |
| 1887 | 2.412753533 | up | 0.000123034 | 0.002948224 | BG507812 | 217 | 13 | 31 | 41 | 3.2 | photomorphogenesis |
| 1888 | 2.414940217 | up | 0.0000539 | 0.001975394 | BF424169 | 433 | 36 | 46 | 78 | 2e−16 | |
| 1889 | 2.415558687 | up | 0.001048506 | 0.010722817 | CF809003 | 704 | 153 | 198 | 77 | 3e−95 | FAD2-2 microsomal omega-6 |
| | | | | | | | | | | | desaturase |
| 1890 | 2.41618069 | up | 0.00069321 | 0.008246168 | CF807504 | 641 | 179 | 212 | 84 | e−101 | glutamate 1-semialdehyde |
| | | | | | | | | | | | aminotransferase |
| 1891 | 2.416351987 | up | 0.0000667 | 0.002171397 | BM887251 | 189 | 9 | 13 | 69 | 3.2 | |
| 1892 | 2.416952765 | up | 0.001071688 | 0.010870137 | CD408191 | 681 | 114 | 121 | 94 | 3e−61 | glycolysis |
| 1893 | 2.417386889 | up | 0.000350913 | 0.005365542 | AW350755 | 655 | 52 | 106 | 49 | 1e−29 | protein biosynthesis |
| 1894 | 2.417695958 | up | 0.000290989 | 0.00479424 | BM178850 | 404 | 31 | 50 | 62 | 4e−13 | |
| 1895 | 2.418943564 | up | 0.0000805 | 0.0023965 | CD396812 | 712 | 82 | 94 | 87 | 1e−40 | fatty acid biosynthesis |
| 1896 | 2.419063153 | up | 0.0000439 | 0.001772209 | CD393016 | 337 | 21 | 58 | 36 | 1.1 | |
| 1897 | 2.419342541 | up | 0.00000668 | 0.000860462 | BF324950 | 420 | 40 | 71 | 56 | 2e−16 | |
| 1898 | 2.419479495 | up | 0.003389603 | 0.022710462 | AW100848 | 338 | 11 | 32 | 34 | 2.5 | protein amino acid phosphorylation |
| 1899 | 2.419937979 | up | 0.000889006 | 0.009614455 | BI699366 | 532 | 32 | 64 | 50 | 1e−11 | |
| 1900 | 2.420768965 | up | 0.000241712 | 0.004319124 | BM092715 | 556 | 126 | 177 | 71 | 1e−64 | |
| 1901 | 2.420929242 | up | 0.00029434 | 0.004824317 | BF595219 | 478 | 36 | 56 | 64 | 5e−19 | |
| 1902 | 2.421196659 | up | 0.001014838 | 0.010499828 | BE658260 | 775 | 151 | 200 | 75 | 3e−82 | transport |
| 1903 | 2.421202813 | up | 0.000379343 | 0.005608036 | BE822480 | 512 | 34 | 100 | 34 | 6e−05 | two-component signal transduction |
| | | | | | | | | | | | system (phosphorelay) |
| 1904 | 2.421222964 | up | 0.00023651 | 0.004262621 | BU545243 | 578 | 57 | 100 | 57 | 3e−28 | |
| 1905 | 2.422287111 | up | 0.000401228 | 0.005813472 | BE209570 | 663 | 165 | 221 | 74 | 8e−98 | protein biosynthesis |
| 1906 | 2.422391803 | up | 0.000139677 | 0.003145136 | CF808180 | 713 | 143 | 171 | 83 | 3e−81 | Vacuolar processing enzyme 2 |
| | | | | | | | | | | | (VPE2), protein catabolism |
| 1907 | 2.422929912 | up | 0.0000354 | 0.001589423 | BU545269 | 498 | 89 | 108 | 82 | 5e−49 | electron transport |
| 1908 | 2.42352725 | up | 0.0000602 | 0.002066371 | BE822001 | 517 | 43 | 73 | 58 | 7e−22 | |
| 1909 | 2.424692216 | up | 0.00010238 | 0.002675093 | BQ473675 | 456 | 71 | 80 | 88 | 6e−38 | transport |
| 1910 | 2.42497335 | up | 0.002259546 | 0.017444696 | CD392870 | 458 | 17 | 51 | 33 | 3.7 | tRNA processing |
| 1911 | 2.425234146 | up | 0.000760778 | 0.00876176 | U40666.1 | | | | | | biotin carboxyl carrier protein |
| | | | | | | | | | | | precursor |
| 1912 | 2.425438898 | up | 0.000396293 | 0.005770876 | BM885392 | 474 | 14 | 33 | 42 | 3.9 | protein amino acid phosphorylation |
| 1913 | 2.426014798 | up | 0.00082609 | 0.009184346 | CD411069 | 216 | 10 | 21 | 47 | 9.4 | regulation of transcription\, DNA- |
| | | | | | | | | | | | dependent |
| 1914 | 2.426645184 | up | 0.003147055 | 0.021672158 | BE800605 | 412 | 15 | 27 | 55 | 0.003 | |
| 1915 | 2.427181353 | up | 0.000154952 | 0.003324117 | BU084442 | 425 | 47 | 73 | 64 | 6e−23 | protein amino acid phosphorylation |
| 1916 | 2.428749456 | up | 0.000941675 | 0.009980326 | BI701943 | 579 | 100 | 139 | 71 | 2e−48 | |
| 1917 | 2.429085247 | up | 0.00000705 | 0.000873658 | BM094097 | 525 | 31 | 54 | 57 | 7e−15 | |
| 1918 | 2.429276148 | up | 0.000627229 | 0.007760718 | CD416491 | 449 | 38 | 45 | 84 | 7e−15 | ossification |
| 1919 | 2.429306081 | up | 0.000134103 | 0.003071963 | BQ453853 | 586 | 66 | 85 | 77 | 4e−33 | purine nucleotide biosynthesis |
| 1920 | 2.430193504 | up | 0.000015 | 0.001128257 | BE347809 | 432 | 100 | 141 | 70 | 2e−49 | RNA processing |
| 1921 | 2.431508228 | up | 0.0000726 | 0.002264259 | CD416202 | 661 | 52 | 93 | 55 | 7e−24 | |
| 1922 | 2.432337428 | up | 0.001502755 | 0.013350171 | CD418000 | 670 | 66 | 149 | 44 | 2e−35 | transport |
| 1923 | 2.432610174 | up | 0.000699813 | 0.008290479 | CD417435 | 530 | 28 | 62 | 45 | 2e−05 | Glutamate 1-semialdehyde |
| | | | | | | | | | | | aminotransferase |
| 1924 | 2.432718791 | up | 0.001648161 | 0.014192749 | AW757083 | 481 | 48 | 60 | 80 | 6e−23 | transport |
| 1925 | 2.433377174 | up | 0.001157738 | 0.011388265 | CF808936 | 552 | 118 | 167 | 70 | 6e−65 | metabolism |
| 1926 | 2.434885849 | up | 0.000568916 | 0.007250504 | CD390753 | 386 | 27 | 38 | 71 | 5e−12 | |
| 1927 | 2.435870684 | up | 0.000130507 | 0.00303449 | AW152939 | 243 | 12 | 22 | 54 | 5.4 | |
| 1928 | 2.43645571 | up | 0.0000982 | 0.002619922 | CA936829 | 575 | 127 | 155 | 81 | 4e−67 | |
| 1929 | 2.436677809 | up | 0.0000151 | 0.001128338 | CA934970 | 357 | 10 | 17 | 58 | 4.3 | |
| 1930 | 2.437139118 | up | 0.0000186 | 0.00122629 | BM520807 | 585 | 107 | 195 | 54 | 2e−55 | |
| 1931 | 2.437904479 | up | 0.0000304 | 0.00148348 | CD412525 | 656 | 75 | 88 | 85 | 1e−37 | electron transport |
| 1932 | 2.438446416 | up | 0.0000289 | 0.001464287 | BE822043 | 644 | 58 | 107 | 54 | 7e−27 | |
| 1933 | 2.438563449 | up | 0.001272464 | 0.012049967 | BU765847 | 549 | 16 | 54 | 29 | 5.2 | protein folding |
| 1934 | 2.43873508 | up | 0.0000186 | 0.00122629 | BI969938 | 747 | 53 | 95 | 55 | 2e−20 | transport |
| 1935 | 2.438779488 | up | 0.000233741 | 0.004241302 | BM188885 | 416 | 58 | 78 | 74 | 4e−31 | |
| 1936 | 2.439234087 | up | 0.001817718 | 0.015126466 | BQ297031 | 523 | 65 | 149 | 43 | 5e−26 | regulation of transcription |
| 1937 | 2.44101357 | up | 0.000899946 | 0.009683106 | CD392544 | 478 | 12 | 29 | 41 | 5.2 | proteolysis and peptidolysis |
| 1938 | 2.441861143 | up | 0.000828499 | 0.009203533 | CD396139 | 538 | 71 | 110 | 64 | 6e−39 | |
| 1939 | 2.442249871 | up | 0.000203612 | 0.003893568 | AW350781 | 785 | 133 | 157 | 84 | 1e−73 | |
| 1940 | 2.442771708 | up | 0.0027535 | 0.019863857 | BU926554 | 554 | 100 | 101 | 99 | 8e−60 | regulation of transcription\, DNA- |
| | | | | | | | | | | | dependent |
| 1941 | 2.443811016 | up | 0.000136034 | 0.003091825 | AI899853 | 474 | 54 | 57 | 94 | 8e−28 | purine nucleotide biosynthesis |
| 1942 | 2.443962352 | up | 0.001200535 | 0.011628744 | BU550191 | 560 | 83 | 111 | 74 | 2e−44 | histidine biosynthesis |
| 1943 | 2.444585099 | up | 0.004397401 | 0.027133175 | AW101688 | 416 | 38 | 54 | 70 | 1e−16 | carbohydrate metabolism |
| 1944 | 2.444925854 | up | 0.0000268 | 0.00140062 | BG045417 | 359 | 22 | 28 | 78 | 2e−09 | lysine biosynthesis via |
| | | | | | | | | | | | diaminopimelate |
| 1945 | 2.445254374 | up | 0.000350292 | 0.005358222 | CD405202 | 692 | 93 | 144 | 64 | 8e−50 | |
| 1946 | 2.445303617 | up | 0.001014923 | 0.010499828 | BQ080721 | 425 | 14 | 41 | 34 | 2.4 | proteolysis and peptidolysis |
| 1947 | 2.44556221 | up | 0.0000242 | 0.001348295 | BU549779 | | | | | | |
| 1948 | 2.446001733 | up | 0.0000473 | 0.001827727 | BG239318 | 405 | 34 | 43 | 79 | 6e−14 | |
| 1949 | 2.448180603 | up | 0.0000104 | 0.000963549 | BQ296723 | 589 | 111 | 145 | 76 | 1e−59 | purine nucleotide biosynthesis |
| 1950 | 2.44821249 | up | 0.0000408 | 0.001731672 | CD400127 | 613 | 96 | 133 | 72 | 1e−50 | protein biosynthesis |
| 1951 | 2.448298926 | up | 0.0000869 | 0.002478118 | BQ786282 | 608 | 90 | 137 | 65 | 2e−52 | apoptosis |
| 1952 | 2.449685778 | up | 0.0000386 | 0.001678647 | AW350338 | 595 | 53 | 79 | 67 | 1e−25 | anion transport |
| 1953 | 2.451219586 | up | 0.000171364 | 0.003531877 | BM519716 | 550 | 30 | 41 | 73 | 1e−13 | proton transport |
| 1954 | 2.452242745 | up | 0.00000454 | 0.000784876 | BG363223 | 583 | 115 | 148 | 77 | 5e−63 | rRNA processing |
| 1955 | 2.452440101 | up | 0.001194474 | 0.011600557 | CD401781 | 707 | 92 | 158 | 58 | 1e−39 | ossification |
| 1956 | 2.45258688 | up | 0.000195021 | 0.003785091 | BE807790 | 408 | 25 | 45 | 55 | 5e−04 | mRNA splicing |
| 1957 | 2.453285234 | up | 0.000181346 | 0.003658009 | CD408020 | 589 | 72 | 137 | 52 | 7e−35 | metabolism |
| 1958 | 2.453445049 | up | 0.0000674 | 0.002177084 | CD391208 | 304 | 23 | 25 | 92 | 2e−07 | protein biosynthesis |
| 1959 | 2.453558359 | up | 0.000449388 | 0.006262193 | BI786194 | 569 | 119 | 135 | 88 | 6e−66 | protein-nucleus import |
| 1960 | 2.453700773 | up | 0.000750376 | 0.008687179 | BE821647 | 560 | 98 | 143 | 68 | 2e−51 | aromatic amino acid family |
| | | | | | | | | | | | biosynthesis\, shikimate pathway |
| 1961 | 2.454137017 | up | 0.003332059 | 0.022523326 | CD406772 | 749 | 77 | 150 | 51 | 8e−36 | metabolism |
| 1962 | 2.454730247 | up | 0.000029 | 0.001465395 | BU548542 | 450 | 78 | 149 | 52 | 3e−34 | vitamin B2 biosynthesis |
| 1963 | 2.45511566 | up | 0.000326307 | 0.005152695 | CD404774 | 622 | 68 | 123 | 55 | 2e−32 | |
| 1964 | 2.455395986 | up | 0.000375534 | 0.005575587 | CF808851 | 556 | 135 | 159 | 84 | 2e−73 | glycolysis |
| 1965 | 2.455417997 | up | 0.000232675 | 0.004227941 | CD402926 | 629 | 73 | 94 | 77 | 2e−36 | fatty acid biosynthesis |
| 1966 | 2.455836239 | up | 0.0000545 | 0.001979942 | BU761875 | 444 | 91 | 109 | 83 | 3e−47 | biological_process unknown |
| 1967 | 2.455961243 | up | 0.000642067 | 0.007861553 | BI786194 | 569 | 119 | 135 | 88 | 6e−66 | protein-nucleus import |
| 1968 | 2.456573105 | up | 0.00026937 | 0.00458997 | BI942877 | 574 | 167 | 188 | 88 | 7e−96 | metabolism |
| 1969 | 2.456985218 | up | 0.000495219 | 0.006642601 | CD404482 | 392 | 17 | 56 | 30 | 2.0 | |
| 1970 | 2.457347798 | up | 0.0000644 | 0.002138008 | CD408141 | 644 | 40 | 65 | 61 | 3e−17 | |
| 1971 | 2.457370608 | up | 0.000278786 | 0.004691844 | CF807934 | 520 | 135 | 162 | 83 | 1e−71 | arginine catabolism |
| 1972 | 2.458444741 | up | 0.000931777 | 0.009906122 | BQ253094 | 546 | 133 | 181 | 73 | 2e−77 | |
| 1973 | 2.458951918 | up | 0.000101965 | 0.002670838 | BG510963 | 506 | 137 | 168 | 81 | 2e−78 | phenylalanyl-tRNA aminoacylation |
| 1974 | 2.459112013 | up | 0.000591817 | 0.007457493 | CD403449 | 675 | 122 | 143 | 85 | 3e−64 | protein biosynthesis |
| 1975 | 2.459550778 | up | 0.000819472 | 0.009135628 | BG154896 | 223 | 14 | 30 | 46 | 0.65 | cytochrome P450 monooxygenase |
| | | | | | | | | | | | CYP83D1, metabolism |
| 1976 | 2.462580145 | up | 0.000165112 | 0.003448378 | BF324751 | 344 | 40 | 47 | 85 | 2e−16 | |
| 1977 | 2.462624578 | up | 0.000315726 | 0.005038495 | CF921737 | 637 | 112 | 166 | 67 | 8e−58 | protein folding |
| 1978 | 2.462678983 | up | 0.0000547 | 0.0019813 | CD416202 | 661 | 52 | 93 | 55 | 7e−24 | |
| 1979 | 2.464306495 | up | 0.0000762 | 0.002325223 | BI427009 | 571 | 131 | 165 | 79 | 2e−69 | transport |
| 1980 | 2.465240973 | up | 0.0000447 | 0.001789988 | BG045417 | 359 | 22 | 28 | 78 | 2e−09 | lysine biosynthesis via |
| | | | | | | | | | | | diaminopimelate |
| 1981 | 2.465241795 | up | 0.0000422 | 0.001755005 | BE659155 | 569 | 60 | 84 | 71 | 6e−31 | mitochondrial translocation |
| 1982 | 2.467121906 | up | 0.000123107 | 0.002948224 | BQ253153 | 495 | 49 | 49 | 100 | 2e−23 | |
| 1983 | 2.467387033 | up | 0.0000513 | 0.001923726 | BU761095 | | | | | | nucleosome assembly protein 1 |
| 1984 | 2.468571619 | up | 0.0000253 | 0.001372273 | AW309551 | 659 | 50 | 99 | 50 | 3e−17 | protein biosynthesis |
| 1985 | 2.468657515 | up | 0.000055 | 0.001986086 | CA801713 | 635 | 172 | 210 | 81 | e−101 | ‘de novo’ pyrimidine base |
| | | | | | | | | | | | biosynthesis |
| 1986 | 2.46899177 | up | 0.0000163 | 0.001156628 | BM178931 | 542 | 138 | 180 | 76 | 2e−80 | |
| 1987 | 2.470028056 | up | 0.0000842 | 0.002451769 | CD395846 | 690 | 91 | 121 | 75 | 5e−50 | |
| 1988 | 2.471625786 | up | 0.000131527 | 0.003040011 | BG652916 | 291 | 15 | 45 | 33 | 1.4 | response to wounding |
| 1989 | 2.472174488 | up | 0.000485568 | 0.006579306 | AW311303 | 431 | 46 | 50 | 92 | 3e−18 | glycolysis |
| 1990 | 2.472882541 | up | 0.000340941 | 0.005277719 | BF070610 | 553 | 29 | 41 | 70 | 3e−13 | regulation of transcription |
| 1991 | 2.473517757 | up | 0.000316321 | 0.00504181 | CA853710 | 429 | 16 | 41 | 39 | 0.13 | |
| 1992 | 2.473736601 | up | 0.001845028 | 0.015249152 | AW459517 | 519 | 160 | 173 | 92 | 3e−91 | |
| 1993 | 2.473928522 | up | 0.0000202 | 0.001260859 | CK606633 | 475 | 23 | 37 | 62 | 3e−07 | |
| 1994 | 2.474884358 | up | 0.000151734 | 0.003287629 | CD487575 | 647 | 82 | 82 | 100 | 1e−40 | nucleosome assembly |
| 1995 | 2.476313225 | up | 0.005448674 | 0.03131977 | CA801759 | 602 | 138 | 202 | 68 | 9e−74 | protein catabolism |
| 1996 | 2.476401623 | up | 0.00000504 | 0.000799682 | BU762685 | 428 | 90 | 146 | 61 | 2e−46 | mRNA catabolism\, nonsense- |
| | | | | | | | | | | | mediated |
| 1997 | 2.476798657 | up | 0.00000685 | 0.000870238 | AW102564 | 243 | 12 | 37 | 32 | 1.4 | |
| 1998 | 2.477060764 | up | 0.0000199 | 0.001255458 | BG352685 | 468 | 120 | 160 | 75 | 1e−66 | |
| 1999 | 2.477072234 | up | 0.0000334 | 0.001546189 | AI856393 | 506 | 30 | 37 | 81 | 8e−13 | |
| 2000 | 2.478218272 | up | 0.00054365 | 0.007029569 | CF806960 | 612 | 158 | 185 | 85 | 1e−88 | |
| 2001 | 2.478265579 | up | 0.0000629 | 0.002110345 | BQ610800 | 552 | 66 | 90 | 73 | 7e−34 | electron transport |
| 2002 | 2.479714732 | up | 0.004850553 | 0.02896869 | BQ081774 | 534 | 164 | 178 | 92 | 1e−92 | |
| 2003 | 2.480212463 | up | 0.000636186 | 0.007822659 | BI943736 | 346 | 18 | 54 | 33 | 0.38 | proteolysis and peptidolysis |
| 2004 | 2.480431124 | up | 0.000155515 | 0.003326183 | CA851201 | 442 | 90 | 116 | 77 | 1e−45 | transport |
| 2005 | 2.480822585 | up | 0.000182324 | 0.003661479 | BM891639 | 562 | 12 | 39 | 30 | 1.9 | |
| 2006 | 2.481773712 | up | 0.000171481 | 0.003532349 | CD406717 | 639 | 42 | 143 | 29 | 2e−13 | ATP biosynthesis |
| 2007 | 2.481835768 | up | 0.000031 | 0.00150215 | BM885863 | 560 | 13 | 46 | 28 | 1.9 | |
| 2008 | 2.482910881 | up | 0.001602567 | 0.013905915 | BE658030 | 449 | 49 | 76 | 64 | 3e−29 | |
| 2009 | 2.485272523 | up | 0.0000277 | 0.001430061 | AW317184 | 672 | 128 | 154 | 83 | 5e−74 | |
| 2010 | 2.485819025 | up | 0.0000257 | 0.001377636 | AW781478 | 641 | 197 | 213 | 92 | e−109 | |
| 2011 | 2.486182451 | up | 0.0000414 | 0.001745262 | AI960030 | 759 | 99 | 139 | 71 | 3e−54 | proteolysis and peptidolysis |
| 2012 | 2.486248658 | up | 0.000416798 | 0.005948834 | CD401340 | 680 | 120 | 140 | 85 | 2e−65 | protein biosynthesis |
| 2013 | 2.487137978 | up | 0.006025652 | 0.033540403 | CA802562 | 734 | 60 | 70 | 85 | 2e−30 | regulation of transcription |
| 2014 | 2.487197707 | up | 0.000150425 | 0.003269358 | X96864.1 | | | | | | phosphoribosylamine--glycine ligase |
| 2015 | 2.488513715 | up | 0.002133611 | 0.01686084 | BU546883 | 606 | 51 | 131 | 38 | 3e−17 | |
| 2016 | 2.488620234 | up | 0.00010487 | 0.002706873 | CD399645 | 623 | 38 | 48 | 79 | 7e−18 | |
| 2017 | 2.489419638 | up | 0.0000125 | 0.001058667 | BU084005 | 568 | 88 | 169 | 52 | 2e−43 | |
| 2018 | 2.491401136 | up | 0.0000118 | 0.001029181 | AW756007 | 612 | 159 | 204 | 77 | 7e−89 | metabolism |
| 2019 | 2.491426018 | up | 0.000732527 | 0.008556911 | BG511051 | 441 | 13 | 34 | 38 | 2.0 | |
| 2020 | 2.494439895 | up | 0.000596071 | 0.00750347 | BQ785938 | 424 | 60 | 71 | 84 | 7e−29 | transport |
| 2021 | 2.494635351 | up | 0.0000581 | 0.00202784 | AW760477 | 406 | 58 | 97 | 59 | 1e−29 | |
| 2022 | 2.494842897 | up | 0.0000946 | 0.002566638 | BM093450 | 479 | 76 | 146 | 52 | 1e−40 | |
| 2023 | 2.497237662 | up | 0.000019 | 0.001231955 | CF805783 | 541 | 97 | 113 | 85 | 6e−51 | |
| 2024 | 2.49837089 | up | 0.008023185 | 0.040557265 | CK605699 | 612 | 48 | 160 | 30 | 6e−06 | |
| 2025 | 2.498560819 | up | 0.001038825 | 0.010664401 | L34346.1 | | | | | | stearoyl-acyl carrier protein |
| | | | | | | | | | | | desaturase |
| 2026 | 2.498836995 | up | 0.004539466 | 0.027709387 | L17319.1 | | | | | | |
| 2027 | 2.500040716 | up | 0.000110552 | 0.002790944 | BQ610045 | 320 | 11 | 23 | 47 | 1.1 | transport |
| 2028 | 2.500449484 | up | 0.000264578 | 0.004540648 | AW706751 | 300 | 38 | 95 | 40 | 5e−07 | |
| 2029 | 2.500576158 | up | 0.000342698 | 0.005289389 | AF255338.1 | | | | | | 20S proteasome subunit |
| 2030 | 2.501606317 | up | 0.00011889 | 0.002892577 | BG237386 | 302 | 12 | 33 | 36 | 1.9 | RNA dependent DNA replication |
| 2031 | 2.503392041 | up | 0.002564558 | 0.018948583 | CF807286 | 567 | 120 | 161 | 74 | 7e−70 | transport |
| 2032 | 2.503603502 | up | 0.00000573 | 0.000820095 | BQ453257 | 354 | 8 | 16 | 50 | 9.6 | electron transport |
| 2033 | 2.504883752 | up | 0.000189114 | 0.003724256 | CF805684 | 554 | 166 | 181 | 91 | 8e−94 | purine nucleotide biosynthesis |
| 2034 | 2.505608841 | up | 0.00030025 | 0.004880547 | BI701497 | 332 | 11 | 13 | 84 | 0.85 | oligopeptide transport |
| 2035 | 2.506824284 | up | 0.000183616 | 0.003675665 | AI443347 | 767 | 134 | 173 | 77 | 3e−72 | protein biosynthesis |
| 2036 | 2.508601669 | up | 0.0000401 | 0.001711701 | CD414787 | 558 | 64 | 154 | 41 | 4e−30 | chemotaxis |
| 2037 | 2.508979245 | up | 0.000206848 | 0.00393544 | AW348176 | 552 | 44 | 65 | 67 | 6e−26 | G-protein coupled receptor protein |
| | | | | | | | | | | | signaling pathway |
| 2038 | 2.509310937 | up | 0.000234257 | 0.004244389 | BG507763 | 385 | 20 | 61 | 32 | 1.8 | |
| 2039 | 2.509670879 | up | 0.0000211 | 0.001283819 | CA803140 | 403 | 28 | 35 | 80 | 2e−11 | transport |
| 2040 | 2.510331334 | up | 0.000116475 | 0.002854123 | BG042200 | 583 | 55 | 91 | 60 | 3e−30 | purine ribonucleoside |
| | | | | | | | | | | | monophosphate biosynthesis |
| 2041 | 2.511177203 | up | 0.000551247 | 0.007103323 | BU549977 | 665 | 104 | 175 | 59 | 9e−57 | regulation of transcription\, DNA- |
| | | | | | | | | | | | dependent |
| 2042 | 2.51126956 | up | 0.000246242 | 0.004368937 | CA802321 | 453 | 79 | 104 | 75 | 5e−40 | biological_process unknown |
| 2043 | 2.511595949 | up | 0.005316432 | 0.030817116 | BM085604 | 565 | 136 | 190 | 71 | 2e−75 | |
| 2044 | 2.51284692 | up | 0.000031 | 0.00150215 | CD394656 | 402 | 56 | 70 | 80 | 5e−29 | |
| 2045 | 2.513534122 | up | 0.000125472 | 0.002970121 | CD405439 | 382 | 23 | 29 | 79 | 9e−08 | |
| 2046 | 2.51482247 | up | 0.00000366 | 0.000764834 | BM731838 | 545 | 80 | 152 | 52 | 5e−38 | |
| 2047 | 2.514916936 | up | 0.0000356 | 0.001591345 | CD399884 | 685 | 39 | 81 | 48 | 5e−17 | regulation of transcription\, DNA- |
| | | | | | | | | | | | dependent |
| 2048 | 2.515303397 | up | 0.000145207 | 0.003201528 | AW432827 | 616 | 82 | 101 | 81 | 2e−42 | protein biosynthesis |
| 2049 | 2.515307006 | up | 0.0000283 | 0.001451074 | CF806086 | 576 | 129 | 194 | 66 | 2e−70 | |
| 2050 | 2.515711178 | up | 0.0000939 | 0.00255704 | CF808154 | 725 | 102 | 185 | 55 | 8e−51 | |
| 2051 | 2.515734722 | up | 0.00001 | 0.000951145 | CD407646 | 653 | 124 | 147 | 84 | 4e−74 | asparagine biosynthesis |
| 2052 | 2.516589165 | up | 0.0000296 | 0.00147316 | BG509486 | 512 | 99 | 170 | 58 | 2e−56 | metabolism |
| 2053 | 2.516884224 | up | 0.0000239 | 0.001340162 | BI970672 | 780 | 44 | 91 | 48 | 7e−17 | |
| 2054 | 2.517267973 | up | 0.00049493 | 0.006641564 | AW734534 | 395 | 18 | 55 | 32 | 1.5 | |
| 2055 | 2.517353899 | up | 0.0000191 | 0.001231955 | BI971155 | 780 | 75 | 117 | 64 | 5e−35 | |
| 2056 | 2.518298309 | up | 0.001323696 | 0.012351646 | CF808835 | 283 | 48 | 54 | 88 | 3e−25 | electron transport |
| 2057 | 2.518387839 | up | 0.000162635 | 0.003419435 | AF068249.1 | | | | | | |
| 2058 | 2.51842425 | up | 0.0000859 | 0.002466308 | BI702093 | 421 | 94 | 95 | 98 | 4e−51 | |
| 2059 | 2.518593615 | up | 0.0000988 | 0.002632822 | CD410890 | 549 | 77 | 81 | 95 | 6e−41 | translational initiation |
| 2060 | 2.519585577 | up | 0.002226716 | 0.017294175 | AW102477 | 359 | 10 | 38 | 26 | 5.8 | |
| 2061 | 2.519975368 | up | 0.001053812 | 0.010759527 | BE802025 | 629 | 134 | 208 | 64 | 6e−73 | carbohydrate metabolism |
| 2062 | 2.520112237 | up | 0.000684598 | 0.008179946 | CA802347 | 404 | 15 | 30 | 50 | 0.029 | |
| 2063 | 2.520547424 | up | 0.000552255 | 0.007106409 | AI901054 | 514 | 70 | 92 | 76 | 4e−36 | regulation of transcription |
| 2064 | 2.52146654 | up | 0.00011358 | 0.002827386 | BG045620 | 438 | 16 | 40 | 40 | 0.30 | response to stress |
| 2065 | 2.521712642 | up | 0.000486072 | 0.00658377 | CF806917 | 702 | 158 | 194 | 81 | 1e−83 | |
| 2066 | 2.523363201 | up | 0.001172493 | 0.011488424 | BI699782 | 574 | 71 | 184 | 38 | 9e−30 | metabolism |
| 2067 | 2.523462325 | up | 0.00000615 | 0.000832282 | BU082870 | 582 | 112 | 157 | 71 | 2e−67 | DNA repair |
| 2068 | 2.523480376 | up | 0.000128456 | 0.003008554 | CF807012 | 492 | 71 | 122 | 58 | 2e−29 | fatty acid biosynthesis |
| 2069 | 2.523910078 | up | 0.0000771 | 0.002341624 | AW348925 | 669 | 88 | 96 | 91 | 4e−46 | |
| 2070 | 2.524244742 | up | 0.000114526 | 0.002833761 | BM093119 | 497 | 29 | 37 | 78 | 5e−12 | |
| 2071 | 2.525366714 | up | 0.000724347 | 0.008495667 | CK606438 | | | | | | |
| 2072 | 2.525897394 | up | 0.000172931 | 0.003549485 | AW757044 | 442 | 21 | 51 | 41 | 0.18 | mRNA splicing |
| 2073 | 2.526835307 | up | 0.0000599 | 0.002064083 | BU762116 | 421 | 16 | 17 | 94 | 0.011 | |
| 2074 | 2.526986477 | up | 0.000174274 | 0.003566452 | CF807307 | 701 | 109 | 154 | 70 | 4e−60 | transport |
| 2075 | 2.527224506 | up | 0.00000282 | 0.000720842 | BF423457 | 437 | 48 | 59 | 81 | 5e−22 | |
| 2076 | 2.527439045 | up | 0.000026 | 0.001380871 | BQ610308 | 264 | 11 | 30 | 36 | 1.4 | |
| 2077 | 2.529087953 | up | 0.003123247 | 0.021551608 | BE023458 | 411 | 13 | 37 | 35 | 3.8 | response to auxin stimulus |
| 2078 | 2.529337747 | up | 0.005664857 | 0.032157852 | CD396438 | 638 | 39 | 62 | 62 | 3e−16 | |
| 2079 | 2.53138434 | up | 0.0000104 | 0.000963549 | CD401423 | 383 | 9 | 30 | 30 | 5.3 | |
| 2080 | 2.53183427 | up | 0.00000781 | 0.000899116 | CD413565 | 341 | 14 | 36 | 38 | 3.2 | |
| 2081 | 2.531913984 | up | 0.000517237 | 0.006827294 | AW782149 | 594 | 150 | 195 | 76 | 2e−84 | amino acid metabolism |
| 2082 | 2.532300866 | up | 0.000834638 | 0.009249871 | AW099283 | 386 | 25 | 39 | 64 | 5e−07 | |
| 2083 | 2.53387091 | up | 0.001515825 | 0.013437718 | BU549268 | 629 | 69 | 79 | 87 | 5e−37 | |
| 2084 | 2.53526342 | up | 0.001433146 | 0.012933387 | BI699341 | 570 | 77 | 87 | 88 | 9e−38 | |
| 2085 | 2.535661015 | up | 0.0000472 | 0.001823768 | AW133381 | 543 | 88 | 127 | 69 | 9e−48 | |
| 2086 | 2.535867998 | up | 0.000104427 | 0.002705181 | AW349406 | | | | | | |
| 2087 | 2.536455309 | up | 0.00000691 | 0.000870238 | BI427054 | 324 | 11 | 23 | 47 | 0.49 | |
| 2088 | 2.536737454 | up | 0.000138633 | 0.003129806 | CD411020 | 578 | 100 | 132 | 75 | 4e−48 | photosynthetic electron transport in |
| | | | | | | | | | | | photosystem I |
| 2089 | 2.536790748 | up | 0.0000501 | 0.001898772 | AW234360 | 496 | 55 | 67 | 82 | 5e−27 | |
| 2090 | 2.537040952 | up | 0.000172891 | 0.003549485 | CF807639 | 538 | 74 | 116 | 63 | 7e−34 | |
| 2091 | 2.537965293 | up | 0.001182078 | 0.011528401 | CA782161 | 409 | 25 | 37 | 67 | 5e−08 | |
| 2092 | 2.538119579 | up | 0.000240362 | 0.004302823 | BG043690 | 375 | 46 | 89 | 51 | 9e−18 | transport |
| 2093 | 2.538614693 | up | 0.0000541 | 0.001976427 | U44850.1 | | | | | | G beta-like protein |
| 2094 | 2.539089202 | up | 0.000794789 | 0.008969456 | AW349728 | 661 | 129 | 173 | 74 | 4e−70 | chloride transport |
| 2095 | 2.539109276 | up | 0.0000964 | 0.002595294 | BM893404 | 511 | 70 | 79 | 88 | 3e−34 | |
| 2096 | 2.539836313 | up | 0.000352019 | 0.005371525 | CD416173 | | | | | | |
| 2097 | 2.539864713 | up | 0.00014618 | 0.003216456 | BE824233 | 691 | 79 | 130 | 60 | 3e−34 | |
| 2098 | 2.540158054 | up | 0.000101245 | 0.002664013 | BU761589 | 321 | 14 | 55 | 25 | 0.64 | apoptosis |
| 2099 | 2.540203745 | up | 0.000122544 | 0.0029434 | BU927382 | 619 | 105 | 176 | 59 | 8e−57 | |
| 2100 | 2.540984885 | up | 0.0000314 | 0.001510393 | BG363105 | 573 | 125 | 160 | 78 | 5e−71 | RNA processing |
| 2101 | 2.542026136 | up | 0.00000851 | 0.000900892 | CA820115 | 563 | 119 | 187 | 63 | 5e−65 | protein biosynthesis |
| 2102 | 2.542580504 | up | 0.0000232 | 0.001330585 | BM732844 | 599 | 69 | 103 | 66 | 9e−44 | |
| 2103 | 2.542636877 | up | 0.000382835 | 0.005633779 | BI785747 | 532 | 23 | 26 | 88 | 1e−08 | two-component signal transduction |
| | | | | | | | | | | | system (phosphorelay) |
| 2104 | 2.542661807 | up | 0.00000306 | 0.000720842 | CD413759 | 666 | 74 | 97 | 76 | 1e−38 | glycolysis |
| 2105 | 2.545238492 | up | 0.000234091 | 0.004243417 | BM885986 | 404 | 45 | 64 | 70 | 2e−21 | |
| 2106 | 2.545315086 | up | 0.000484596 | 0.006571609 | AW567674 | 226 | 10 | 21 | 47 | 7.2 | |
| 2107 | 2.545646477 | up | 0.000168769 | 0.003497564 | AW310005 | 652 | 109 | 140 | 77 | 4e−60 | |
| 2108 | 2.546751995 | up | 0.0000805 | 0.0023965 | BE658655 | 354 | 14 | 22 | 63 | 0.009 | |
| 2109 | 2.54782268 | up | 0.001334468 | 0.012409068 | CA938600 | 318 | 24 | 47 | 51 | 1e−06 | |
| 2110 | 2.548742526 | up | 0.000263136 | 0.00452565 | CF809130 | 716 | 91 | 196 | 46 | 4e−39 | cystathionine-gamma-synthase |
| | | | | | | | | | | | precursor, methionine biosynthesis |
| 2111 | 2.549335193 | up | 0.0000228 | 0.001322079 | BI425010 | 518 | 111 | 172 | 64 | 1e−55 | |
| 2112 | 2.54995073 | up | 0.000103973 | 0.002697141 | CD390907 | 647 | 89 | 121 | 73 | 4e−46 | |
| 2113 | 2.550555358 | up | 0.002973783 | 0.020872528 | AI748030 | 369 | 14 | 39 | 35 | 1.7 | asparaginyl-tRNA aminoacylation |
| 2114 | 2.550665062 | up | 0.000073 | 0.00226999 | BE058866 | 478 | 24 | 42 | 57 | 5e−08 | anion transport |
| 2115 | 2.551180734 | up | 0.0000723 | 0.002259699 | AW349898 | 786 | 87 | 151 | 57 | 4e−45 | |
| 2116 | 2.551915016 | up | 0.00012119 | 0.00292207 | CF807639 | 538 | 74 | 116 | 63 | 7e−34 | |
| 2117 | 2.55265857 | up | 0.0000161 | 0.0011509 | AW349278 | 805 | 88 | 134 | 65 | 5e−44 | protein modification |
| 2118 | 2.554041671 | up | 0.000290591 | 0.004791887 | CD415960 | 607 | 75 | 90 | 83 | 2e−34 | |
| 2119 | 2.55423044 | up | 0.0000181 | 0.001219817 | BU091148 | 360 | 16 | 40 | 40 | 0.52 | protein amino acid glycosylation |
| 2120 | 2.555292529 | up | 0.000017 | 0.001184989 | AW459165 | 627 | 133 | 208 | 63 | 9e−80 | |
| 2121 | 2.558345039 | up | 0.000141008 | 0.003154302 | BU925875 | 559 | 73 | 85 | 85 | 5e−36 | adenine salvage pathway |
| 2122 | 2.558791843 | up | 0.001108 | 0.011072154 | BE347576 | 492 | 22 | 61 | 36 | 8e−04 | |
| 2123 | 2.559024176 | up | 0.000426633 | 0.006035098 | CD409536 | 537 | 11 | 23 | 47 | 8.7 | fatty acid beta-oxidation |
| 2124 | 2.561414067 | up | 0.001060731 | 0.010797927 | CA852600 | 321 | 13 | 36 | 36 | 2.4 | |
| 2125 | 2.561606604 | up | 0.001176986 | 0.011510963 | CF805904 | 375 | 50 | 62 | 80 | 3e−23 | |
| 2126 | 2.564524699 | up | 0.000324985 | 0.005136123 | BG042411 | 407 | 65 | 87 | 74 | 8e−32 | |
| 2127 | 2.564600013 | up | 0.002748655 | 0.019848524 | BE823473 | 666 | 96 | 141 | 68 | 5e−52 | chloride transport |
| 2128 | 2.564722212 | up | 0.0000128 | 0.001073795 | BQ611713 | 400 | 13 | 49 | 26 | 5.9 | multidrug transport |
| 2129 | 2.565024211 | up | 0.001196698 | 0.011610166 | CF806914 | 709 | 78 | 248 | 31 | 2e−30 | metabolism |
| 2130 | 2.565512623 | up | 0.00000918 | 0.000924406 | CD392598 | 612 | 14 | 31 | 45 | 1.3 | |
| 2131 | 2.566906371 | up | 0.0000268 | 0.001397859 | CD400086 | 734 | 89 | 97 | 91 | 7e−48 | protein biosynthesis |
| 2132 | 2.570017323 | up | 0.000331925 | 0.005208612 | BU545441 | 473 | 64 | 81 | 79 | 4e−34 | |
| 2133 | 2.572212961 | up | 0.000205334 | 0.003912571 | CA801700 | 488 | 32 | 40 | 80 | 1e−12 | translational elongation |
| 2134 | 2.573138553 | up | 0.0000417 | 0.001750979 | BG238739 | 389 | 56 | 60 | 93 | 3e−24 | |
| 2135 | 2.574115419 | up | 0.000246399 | 0.004369669 | CD415960 | 607 | 75 | 90 | 83 | 2e−34 | |
| 2136 | 2.574463785 | up | 0.000294624 | 0.004826557 | BM891443 | 555 | 26 | 37 | 70 | 9e−08 | ATP/ADP exchange |
| 2137 | 2.577542963 | up | 0.000118336 | 0.002883786 | CF806897 | 716 | 132 | 185 | 71 | 1e−70 | |
| 2138 | 2.577567795 | up | 0.000150445 | 0.003269358 | BU090309 | 283 | 31 | 69 | 44 | 6e−07 | G-protein coupled receptor protein |
| | | | | | | | | | | | signaling pathway |
| 2139 | 2.580074036 | up | 0.00186218 | 0.01532692 | CK606334 | 717 | 118 | 208 | 56 | 1e−65 | Glutathione S-transferase GST 8, |
| | | | | | | | | | | | transport |
| 2140 | 2.580327419 | up | 0.0000254 | 0.001374317 | BG154569 | 280 | 11 | 27 | 40 | 4.2 | |
| 2141 | 2.580420061 | up | 0.000183907 | 0.003679516 | BM308960 | 502 | 21 | 75 | 28 | 2.0 | |
| 2142 | 2.580803789 | up | 0.000103423 | 0.002690367 | BE330842 | 617 | 124 | 183 | 67 | 4e−65 | |
| 2143 | 2.581074011 | up | 0.0000854 | 0.002466308 | CF806170 | 704 | 97 | 171 | 56 | 3e−56 | |
| 2144 | 2.581654915 | up | 0.000232129 | 0.004220052 | AW202087 | 266 | 47 | 64 | 73 | 1e−21 | carbohydrate metabolism |
| 2145 | 2.581905463 | up | 0.0000603 | 0.002066371 | BI970858 | 730 | 107 | 145 | 73 | 4e−60 | purine salvage |
| 2146 | 2.582493609 | up | 0.000379232 | 0.005608036 | BM891556 | 561 | 100 | 197 | 50 | 5e−43 | |
| 2147 | 2.583675662 | up | 0.0000458 | 0.001805817 | CD397282 | 592 | 95 | 105 | 90 | 3e−54 | protein biosynthesis |
| 2148 | 2.584329166 | up | 0.000336101 | 0.00524569 | BF597220 | 603 | 159 | 201 | 79 | 1e−97 | carbohydrate metabolism |
| 2149 | 2.584424409 | up | 0.0000466 | 0.00181354 | BE610287 | 456 | 29 | 42 | 69 | 8e−14 | |
| 2150 | 2.584530141 | up | 0.000204226 | 0.003901743 | AI443284 | 409 | 53 | 107 | 49 | 8e−21 | |
| 2151 | 2.584693739 | up | 0.000022 | 0.001310737 | BM891968 | 450 | 56 | 127 | 44 | 1e−17 | |
| 2152 | 2.58587341 | up | 0.0000115 | 0.001013778 | BU549130 | 601 | 12 | 38 | 31 | 2.1 | |
| 2153 | 2.587916345 | up | 0.00003 | 0.00147742 | CA783226 | 752 | 187 | 245 | 76 | e−110 | electron transport |
| 2154 | 2.588092279 | up | 0.00018173 | 0.003658009 | BQ454170 | 303 | 15 | 50 | 30 | 0.85 | |
| 2155 | 2.588127406 | up | 0.000553311 | 0.007107221 | AW349088 | 652 | 36 | 45 | 80 | 1e−13 | |
| 2156 | 2.589000259 | up | 0.0000253 | 0.001372273 | CF807636 | 700 | 155 | 216 | 71 | 6e−88 | nucleosome assembly |
| 2157 | 2.589472626 | up | 0.000124098 | 0.002952452 | CD406757 | 609 | 76 | 127 | 59 | 1e−39 | histidine biosynthesis |
| 2158 | 2.589579466 | up | 0.000760168 | 0.00876176 | CD400121 | 693 | 158 | 185 | 85 | 2e−87 | protein biosynthesis |
| 2159 | 2.589769615 | up | 0.0000054 | 0.000812263 | CD407712 | 527 | 16 | 40 | 40 | 4.9 | |
| 2160 | 2.590236036 | up | 0.0000378 | 0.001657189 | CD402963 | 422 | 39 | 42 | 92 | 1e−19 | |
| 2161 | 2.590360721 | up | 0.00000752 | 0.000889839 | AW432659 | 505 | 114 | 166 | 68 | 8e−64 | transport |
| 2162 | 2.591067423 | up | 0.000278957 | 0.00469262 | BU964869 | 603 | 66 | 78 | 84 | 1e−37 | intracellular protein transport |
| 2163 | 2.591384592 | up | 0.0000575 | 0.002021756 | BM527360 | 545 | 145 | 178 | 81 | 6e−83 | |
| 2164 | 2.594563348 | up | 0.000197034 | 0.003810395 | AW156783 | 394 | 14 | 30 | 46 | 0.68 | protein amino acid phosphorylation |
| 2165 | 2.594710739 | up | 0.00000665 | 0.000860462 | BF070056 | 648 | 51 | 119 | 42 | 1e−17 | |
| 2166 | 2.595217203 | up | 0.000295011 | 0.004828777 | CF808533 | 733 | 231 | 233 | 99 | e−135 | selenocysteine incorporation |
| 2167 | 2.595408426 | up | 0.000125852 | 0.002973508 | CD417964 | 590 | 75 | 87 | 86 | 1e−42 | |
| 2168 | 2.595863606 | up | 0.000064 | 0.002134923 | CK605629 | 774 | 163 | 222 | 73 | 5e−88 | |
| 2169 | 2.596586365 | up | 0.000286477 | 0.004761612 | BG511047 | | | | | | |
| 2170 | 2.599563996 | up | 0.00019862 | 0.003833184 | CD407817 | 657 | 92 | 152 | 60 | 3e−49 | |
| 2171 | 2.600996394 | up | 0.001178377 | 0.011516578 | AW705556 | 438 | 79 | 146 | 54 | 3e−34 | |
| 2172 | 2.601503892 | up | 0.0000581 | 0.00202784 | BI943988 | 304 | 74 | 75 | 98 | 3e−39 | |
| 2173 | 2.602374987 | up | 0.000250548 | 0.004407052 | CD407770 | 561 | 10 | 22 | 45 | 4.2 | |
| 2174 | 2.60305758 | up | 0.000117428 | 0.002873743 | CF809359 | 636 | 107 | 122 | 87 | 9e−56 | protein biosynthesis |
| 2175 | 2.603490704 | up | 0.00094028 | 0.009972351 | CF806688 | 716 | 185 | 217 | 85 | e−109 | |
| 2176 | 2.604198426 | up | 0.0002722 | 0.004619365 | CB063343 | 476 | 52 | 62 | 83 | 2e−26 | |
| 2177 | 2.604302357 | up | 0.00016914 | 0.003500454 | BU964510 | 620 | 145 | 203 | 71 | 3e−76 | |
| 2178 | 2.604420785 | up | 0.001701588 | 0.014481366 | CA802955 | 289 | 9 | 19 | 47 | 5.4 | |
| 2179 | 2.604577726 | up | 0.000095 | 0.002566638 | AW350048 | 819 | 116 | 177 | 65 | 4e−56 | ribosomal protein S6 |
| 2180 | 2.604732858 | up | 0.000141116 | 0.003154302 | BG511559 | 322 | 9 | 16 | 56 | 2.4 | DNA metabolism |
| 2181 | 2.605393225 | up | 0.0000368 | 0.001628134 | BE023190 | 353 | 33 | 78 | 42 | 5e−11 | protein biosynthesis |
| 2182 | 2.605479408 | up | 0.00165705 | 0.014230199 | BU762579 | 371 | 24 | 32 | 75 | 1e−10 | pyrimidine base metabolism |
| 2183 | 2.605674451 | up | 0.003189462 | 0.021854343 | CA802562 | 734 | 60 | 70 | 85 | 2e−30 | regulation of transcription |
| 2184 | 2.606464938 | up | 0.003302786 | 0.022383966 | BG041563 | 247 | 44 | 51 | 86 | 2e−19 | aromatic compound metabolism |
| 2185 | 2.606518279 | up | 0.000127695 | 0.002999176 | BG238127 | 453 | 93 | 146 | 63 | 3e−54 | |
| 2186 | 2.608038946 | up | 0.0004655 | 0.00638895 | BG362784 | 548 | 115 | 179 | 64 | 6e−60 | |
| 2187 | 2.609155137 | up | 0.0000214 | 0.001291701 | BU547221 | 694 | 150 | 171 | 87 | 3e−81 | DNA repair |
| 2188 | 2.60964407 | up | 0.000870195 | 0.009475313 | CD402482 | 664 | 121 | 134 | 90 | 2e−66 | similar to XP_533674.1 similar to |
| | | | | | | | | | | | ribosomal protein S16 [Canis |
| | | | | | | | | | | | familiaris] |
| 2189 | 2.609680774 | up | 0.00000373 | 0.000764834 | CF807256 | 577 | 145 | 192 | 75 | 3e−84 | defense response |
| 2190 | 2.609784277 | up | 0.001394063 | 0.012742874 | BE584346 | 576 | 100 | 186 | 53 | 2e−55 | |
| 2191 | 2.610045302 | up | 0.0000117 | 0.00102191 | CD396583 | 516 | 49 | 61 | 80 | 9e−25 | |
| 2192 | 2.610291193 | up | 0.000686469 | 0.008194336 | CF806666 | 512 | 96 | 112 | 85 | 3e−52 | nucleotide-sugar metabolism |
| 2193 | 2.611090323 | up | 0.0000328 | 0.001543897 | AW203473 | 370 | 49 | 84 | 58 | 4e−24 | |
| 2194 | 2.611318656 | up | 0.002294231 | 0.017589729 | U08383.1 | | | | | | BiP isoform B |
| 2195 | 2.61218848 | up | 0.0000313 | 0.001507843 | BG882102 | 534 | 125 | 178 | 70 | 5e−70 | |
| 2196 | 2.612864033 | up | 0.000159546 | 0.003371431 | BG509157 | 392 | 20 | 53 | 37 | 7.5 | |
| 2197 | 2.613064375 | up | 0.000226289 | 0.004147977 | CF807294 | 710 | 191 | 204 | 93 | e−109 | |
| 2198 | 2.614907226 | up | 0.000408187 | 0.005867922 | AW780481 | 346 | 17 | 51 | 33 | 2.5 | metabolism |
| 2199 | 2.615094938 | up | 0.000290418 | 0.004791125 | BU550402 | 544 | 116 | 120 | 96 | 2e−64 | |
| 2200 | 2.61545019 | up | 0.0000105 | 0.00096796 | AW309658 | 617 | 93 | 107 | 86 | 4e−50 | |
| 2201 | 2.616057162 | up | 0.0000217 | 0.001304493 | BE821817 | 593 | 73 | 89 | 82 | 3e−39 | electron transport |
| 2202 | 2.619018995 | up | 0.0000663 | 0.002164602 | CD396884 | 314 | 32 | 35 | 91 | 1e−13 | protein biosynthesis |
| 2203 | 2.619217241 | up | 0.0000129 | 0.001076637 | AI437500 | 438 | 54 | 159 | 33 | 1e−17 | regulation of translation |
| 2204 | 2.619946422 | up | 0.000785984 | 0.008934424 | AW705534 | 286 | 10 | 14 | 71 | 5.4 | malate dehydrogenase, metabolism |
| 2205 | 2.620050004 | up | 0.000021 | 0.001283285 | AW309474 | 645 | 17 | 46 | 36 | 1.1 | sphingosine biosynthesis |
| 2206 | 2.620368619 | up | 0.001624436 | 0.014026983 | CD412043 | 425 | 11 | 25 | 44 | 0.019 | |
| 2207 | 2.625231506 | up | 0.0000695 | 0.002201757 | CF806113 | 545 | 103 | 125 | 82 | 1e−55 | |
| 2208 | 2.625663343 | up | 0.0000162 | 0.001155861 | BE347476 | 553 | 160 | 183 | 87 | 1e−89 | selenocysteine incorporation |
| 2209 | 2.6260555 | up | 0.000366203 | 0.005485846 | BI094895 | 428 | 16 | 23 | 69 | 0.001 | |
| 2210 | 2.626710477 | up | 0.000753054 | 0.008704791 | CF806804 | 137 | 10 | 25 | 40 | 4.2 | |
| 2211 | 2.627561994 | up | 0.0000563 | 0.002005588 | CD412255 | 587 | 52 | 56 | 92 | 4e−28 | protein biosynthesis |
| 2212 | 2.627671655 | up | 0.000855864 | 0.009391032 | CA937360 | 391 | 24 | 44 | 54 | 4e−06 | regulation of transcription |
| 2213 | 2.627806659 | up | 0.0000329 | 0.001543897 | CA800380 | 750 | 69 | 92 | 75 | 4e−35 | transport |
| 2214 | 2.627853201 | up | 0.0000239 | 0.00133979 | CF806126 | | | | | | |
| 2215 | 2.628128533 | up | 0.00015095 | 0.003278189 | CD408222 | 617 | 13 | 43 | 30 | 6.5 | leaf morphogenesis |
| 2216 | 2.628249024 | up | 0.0000752 | 0.002304812 | CF807985 | 311 | 94 | 103 | 91 | 3e−53 | |
| 2217 | 2.628539593 | up | 0.000648492 | 0.007909869 | CD398369 | 647 | 71 | 107 | 66 | 2e−34 | |
| 2218 | 2.62890225 | up | 0.000964889 | 0.010114641 | CF806688 | 716 | 185 | 217 | 85 | e−109 | |
| 2219 | 2.629018159 | up | 0.000918124 | 0.009808101 | CA937360 | 391 | 24 | 44 | 54 | 4e−06 | regulation of transcription |
| 2220 | 2.629831676 | up | 0.000230636 | 0.004196971 | AW755309 | 490 | 36 | 111 | 32 | 6e−12 | |
| 2221 | 2.63191044 | up | 0.010035521 | 0.047113841 | BU090282 | 644 | 112 | 186 | 60 | 2e−57 | |
| 2222 | 2.632157721 | up | 0.000022 | 0.001310737 | L46848.1 | | | | | | acidic ribosomal protein P0 |
| 2223 | 2.632897308 | up | 0.000211676 | 0.003992965 | L14930.1 | | | | | | (Rab7p) |
| 2224 | 2.633274864 | up | 0.000186432 | 0.003694813 | CA819005 | 554 | 109 | 183 | 59 | 5e−60 | regulation of transcription\, DNA- |
| | | | | | | | | | | | dependent |
| 2225 | 2.633322046 | up | 0.000288587 | 0.004773484 | CD411020 | 578 | 100 | 132 | 75 | 4e−48 | photosynthetic electron transport in |
| | | | | | | | | | | | photosystem I |
| 2226 | 2.63456558 | up | 0.0000845 | 0.002456504 | BI426730 | 585 | 40 | 55 | 72 | 1e−14 | ATP/ADP exchange |
| 2227 | 2.635583539 | up | 0.0000847 | 0.002458724 | BE583478 | 768 | 152 | 176 | 86 | 7e−82 | protein biosynthesis |
| 2228 | 2.637766798 | up | 0.008476817 | 0.042130945 | CD411653 | 645 | 31 | 45 | 68 | 6e−12 | |
| 2229 | 2.637812303 | up | 0.00000924 | 0.000924406 | BE823826 | 579 | 38 | 64 | 59 | 6e−15 | cation transport |
| 2230 | 2.638818392 | up | 0.0000993 | 0.002639291 | CD391361 | 698 | 152 | 177 | 85 | 9e−87 | protein biosynthesis |
| 2231 | 2.642487308 | up | 0.0000297 | 0.00147316 | BI973739 | 548 | 162 | 182 | 89 | 7e−98 | potassium ion transport |
| 2232 | 2.642522629 | up | 0.0000105 | 0.00096796 | CD401560 | 626 | 13 | 24 | 54 | 1.8 | |
| 2233 | 2.643536472 | up | 0.000129256 | 0.003021647 | AW317674 | 557 | 89 | 162 | 54 | 4e−43 | cell growth and/or maintenance |
| 2234 | 2.643755768 | up | 0.0000262 | 0.001380871 | CA783355 | 776 | 153 | 247 | 61 | 7e−85 | |
| 2235 | 2.646641844 | up | 0.000146405 | 0.003216456 | CF807089 | 444 | 48 | 81 | 59 | 2e−21 | |
| 2236 | 2.646883683 | up | 0.000115718 | 0.002848619 | BI971561 | 737 | 64 | 167 | 38 | 6e−20 | |
| 2237 | 2.64701596 | up | 0.000238986 | 0.004290802 | CD410890 | 549 | 77 | 81 | 95 | 6e−41 | translational initiation |
| 2238 | 2.647113883 | up | 0.001047119 | 0.010714455 | BU544984 | 675 | 112 | 132 | 84 | 1e−59 | tricarboxylic acid cycle |
| 2239 | 2.648028126 | up | 0.0000725 | 0.002264259 | BE331146 | 336 | 48 | 113 | 42 | 4e−24 | |
| 2240 | 2.649378482 | up | 0.001286021 | 0.012150819 | BI970816 | 771 | 15 | 42 | 35 | 3.2 | intracellular signaling cascade |
| 2241 | 2.650278983 | up | 0.0000736 | 0.00228073 | BM523459 | 589 | 44 | 128 | 34 | 4e−17 | |
| 2242 | 2.650488048 | up | 0.00000313 | 0.000720842 | AI461128 | 505 | 58 | 133 | 43 | 1e−26 | |
| 2243 | 2.650646748 | up | 0.001048197 | 0.01072257 | AW351062 | 451 | 12 | 40 | 30 | 0.41 | |
| 2244 | 2.65325075 | up | 0.000929486 | 0.009898321 | BG725261 | 480 | 89 | 158 | 56 | 4e−51 | |
| 2245 | 2.654255944 | up | 0.001133102 | 0.011231327 | BU089661 | 480 | 24 | 69 | 34 | 2e−04 | regulation of transcription |
| 2246 | 2.656004119 | up | 0.000520178 | 0.006848157 | AI443516 | 253 | 8 | 20 | 40 | 0.39 | protein amino acid phosphorylation |
| 2247 | 2.656099486 | up | 0.0000418 | 0.001750979 | BM886957 | | | | | | |
| 2248 | 2.656554804 | up | 0.0000231 | 0.001328603 | BG650774 | 438 | 55 | 94 | 58 | 5e−27 | |
| 2249 | 2.657042967 | up | 0.00140383 | 0.012789106 | CD412522 | 639 | 52 | 79 | 65 | 2e−29 | |
| 2250 | 2.657365467 | up | 0.000452307 | 0.006289486 | BG551394 | 475 | 49 | 57 | 85 | 5e−24 | |
| 2251 | 2.658051419 | up | 0.0000227 | 0.001321932 | BM568357 | 585 | 57 | 80 | 71 | 2e−29 | |
| 2252 | 2.658144944 | up | 0.0000142 | 0.001112691 | BU763425 | 596 | 27 | 57 | 47 | 2e−07 | electron transport |
| 2253 | 2.658802893 | up | 0.00000163 | 0.000704945 | CD392639 | 423 | 27 | 45 | 60 | 1e−06 | |
| 2254 | 2.658935995 | up | 0.0000227 | 0.001321932 | BG652465 | 420 | 19 | 30 | 63 | 8e−06 | dTDP biosynthesis |
| 2255 | 2.659347951 | up | 0.002355803 | 0.017894018 | CA935531 | 412 | 13 | 31 | 41 | 2.2 | |
| 2256 | 2.659704062 | up | 0.0000239 | 0.00133979 | CD405216 | | | | | | |
| 2257 | 2.659772612 | up | 0.000131328 | 0.003039857 | CA801466 | 725 | 119 | 157 | 75 | 2e−60 | |
| 2258 | 2.660108575 | up | 0.000297371 | 0.004852627 | BU926695 | 390 | 33 | 44 | 75 | 3e−15 | |
| 2259 | 2.660232194 | up | 0.0000451 | 0.001793484 | CF805684 | 554 | 166 | 181 | 91 | 8e−94 | purine nucleotide biosynthesis |
| 2260 | 2.661097782 | up | 0.00000202 | 0.000707618 | BE346738 | 481 | 101 | 160 | 63 | 2e−61 | proteolysis and peptidolysis |
| 2261 | 2.662333332 | up | 0.000247869 | 0.004383337 | AW756603 | 461 | 71 | 113 | 62 | 2e−41 | regulation of transcription |
| 2262 | 2.662399717 | up | 0.0000332 | 0.001543897 | CD404084 | 556 | 35 | 63 | 55 | 8e−09 | regulation of transcription |
| 2263 | 2.662531478 | up | 0.00000313 | 0.000720842 | CF808480 | 428 | 39 | 105 | 37 | 2e−10 | |
| 2264 | 2.663621302 | up | 0.00000548 | 0.000814881 | BU577357 | 433 | 55 | 67 | 82 | 8e−25 | |
| 2265 | 2.663823456 | up | 0.0000919 | 0.00254498 | CD391354 | 616 | 66 | 72 | 91 | 1e−31 | |
| 2266 | 2.664338834 | up | 0.000181501 | 0.003658009 | CD408154 | 724 | 66 | 148 | 44 | 1e−31 | |
| 2267 | 2.664408775 | up | 0.000189727 | 0.003732626 | BI945133 | 322 | 18 | 49 | 36 | 0.075 | regulation of transcription\, DNA- |
| | | | | | | | | | | | dependent |
| 2268 | 2.665110148 | up | 0.000261462 | 0.004506983 | CD391361 | 698 | 152 | 177 | 85 | 9e−87 | protein biosynthesis |
| 2269 | 2.667176487 | up | 0.00000425 | 0.000772527 | AW277529 | 618 | 58 | 119 | 48 | 2e−19 | |
| 2270 | 2.669333069 | up | 0.0000325 | 0.001543897 | BM519579 | 287 | 18 | 57 | 31 | 1.1 | transport |
| 2271 | 2.671029309 | up | 0.000153991 | 0.003321235 | BU927530 | 609 | 65 | 148 | 43 | 3e−31 | |
| 2272 | 2.671769249 | up | 0.0002503 | 0.004407052 | BE821831 | 638 | 139 | 157 | 88 | 1e−75 | |
| 2273 | 2.671794291 | up | 0.0000511 | 0.001918982 | BQ454115 | 326 | 17 | 63 | 26 | 1.8 | |
| 2274 | 2.67220389 | up | 0.000111359 | 0.002796165 | AW568804 | 292 | 25 | 39 | 64 | 9e−08 | protein biosynthesis |
| 2275 | 2.673292003 | up | 0.000291523 | 0.004796737 | CF809073 | 468 | 140 | 155 | 90 | 6e−81 | |
| 2276 | 2.673772165 | up | 0.0000443 | 0.001779937 | BQ740282 | 511 | 61 | 106 | 57 | 4e−30 | protein biosynthesis |
| 2277 | 2.674009995 | up | 0.000109029 | 0.002775649 | AW351051 | 752 | 157 | 189 | 83 | 2e−88 | protein biosynthesis |
| 2278 | 2.674352927 | up | 0.000581681 | 0.00736431 | BI785461 | 513 | 14 | 37 | 37 | 0.42 | regulation of transcription\, DNA- |
| | | | | | | | | | | | dependent |
| 2279 | 2.676010864 | up | 0.0000287 | 0.001464125 | L38856.1 | | | | | | nucleosome assembly protein 1 |
| 2280 | 2.676457528 | up | 0.000058 | 0.00202784 | BI426730 | 585 | 40 | 55 | 72 | 1e−14 | ATP/ADP exchange |
| 2281 | 2.677521039 | up | 0.00014822 | 0.003240109 | BE209656 | 479 | 50 | 62 | 80 | 6e−23 | |
| 2282 | 2.678426554 | up | 0.0000468 | 0.00182046 | AW395282 | 622 | 40 | 111 | 36 | 6e−14 | carbohydrate metabolism |
| 2283 | 2.680468429 | up | 0.003364235 | 0.022626124 | CD397432 | 649 | 33 | 74 | 44 | 7e−05 | |
| 2284 | 2.680538261 | up | 0.0000662 | 0.002164602 | CD408823 | 686 | 77 | 105 | 73 | 5e−39 | protein biosynthesis |
| 2285 | 2.680875904 | up | 0.003298585 | 0.022359518 | BE021487 | 232 | 11 | 21 | 52 | 5.5 | |
| 2286 | 2.681700751 | up | 0.00071853 | 0.008444527 | AW186100 | 652 | 156 | 217 | 71 | 2e−92 | |
| 2287 | 2.682518207 | up | 0.001301192 | 0.012238819 | BM121509 | 870 | 58 | 125 | 46 | 1e−26 | |
| 2288 | 2.682767769 | up | 0.0000108 | 0.000981585 | BG155858 | 458 | 32 | 43 | 74 | 2e−11 | |
| 2289 | 2.684168562 | up | 0.0000471 | 0.001823768 | CF806931 | 710 | 133 | 157 | 84 | 7e−68 | ossification |
| 2290 | 2.684934101 | up | 0.0000696 | 0.002203792 | CD409698 | 533 | 57 | 110 | 51 | 5e−27 | response to UV |
| 2291 | 2.685937421 | up | 0.0000408 | 0.001731672 | BM270176 | 295 | 18 | 58 | 31 | 0.66 | |
| 2292 | 2.687004598 | up | 0.000207739 | 0.003946402 | CD415960 | 607 | 75 | 90 | 83 | 2e−34 | |
| 2293 | 2.68717485 | up | 0.008378947 | 0.041854677 | BI321495 | 375 | 15 | 39 | 38 | 7e−04 | |
| 2294 | 2.688278474 | up | 0.00458783 | 0.0278828 | AW471758 | 213 | 15 | 57 | 26 | 4.2 | |
| 2295 | 2.688975967 | up | 0.000133095 | 0.003060044 | BF598488 | 423 | 54 | 89 | 60 | 4e−24 | leaf morphogenesis |
| 2296 | 2.690866488 | up | 0.000372819 | 0.005549349 | BM091754 | | | | | | |
| 2297 | 2.691463263 | up | 0.001031992 | 0.010623251 | BU545558 | 480 | 58 | 72 | 80 | 2e−28 | electron transport |
| 2298 | 2.692181417 | up | 0.000449292 | 0.006262193 | AW101209 | 430 | 21 | 58 | 36 | 0.49 | G-protein coupled receptor protein |
| | | | | | | | | | | | signaling pathway |
| 2299 | 2.692866932 | up | 0.0000199 | 0.001255458 | BI969785 | 686 | 121 | 168 | 72 | 3e−66 | |
| 2300 | 2.694735258 | up | 0.000016 | 0.0011509 | CD391381 | 597 | 64 | 110 | 58 | 2e−26 | proteolysis and peptidolysis |
| 2301 | 2.69495372 | up | 0.0000303 | 0.001483425 | CD396304 | 722 | 83 | 108 | 76 | 2e−43 | galactose metabolism |
| 2302 | 2.695444692 | up | 0.00000513 | 0.000800719 | BF596398 | 481 | 66 | 93 | 70 | 5e−32 | asparagine biosynthesis |
| 2303 | 2.696488761 | up | 0.000217493 | 0.004059301 | CK606547 | 423 | 41 | 45 | 91 | 2e−20 | protein biosynthesis |
| 2304 | 2.696696385 | up | 0.0000082 | 0.000900527 | BM731752 | 480 | 59 | 86 | 68 | 9e−32 | |
| 2305 | 2.697168499 | up | 0.0000618 | 0.002093668 | CF807343 | 655 | 144 | 198 | 72 | 3e−79 | response to wounding |
| 2306 | 2.697743351 | up | 0.000727257 | 0.008521103 | CD413578 | 633 | 49 | 73 | 67 | 3e−20 | |
| 2307 | 2.698347842 | up | 0.000433264 | 0.006089987 | CD392909 | 664 | 13 | 39 | 33 | 9.6 | DNA recombination |
| 2308 | 2.700694194 | up | 0.000591191 | 0.007454594 | BI469627 | 431 | 36 | 48 | 75 | 2e−16 | |
| 2309 | 2.700895441 | up | 0.000144239 | 0.003182607 | BI968107 | 789 | 12 | 44 | 27 | 3.3 | |
| 2310 | 2.701482308 | up | 0.0000215 | 0.001295711 | BG511394 | 351 | 43 | 75 | 57 | 4e−17 | |
| 2311 | 2.702054079 | up | 0.0000789 | 0.00236758 | CF805792 | 503 | 48 | 53 | 90 | 8e−24 | |
| 2312 | 2.703606211 | up | 0.000301781 | 0.004892759 | AW596896 | 423 | 52 | 103 | 50 | 1e−22 | |
| 2313 | 2.704790239 | up | 0.0000115 | 0.001013808 | CA784082 | 570 | 10 | 15 | 66 | 4.3 | |
| 2314 | 2.705107404 | up | 0.00000196 | 0.000707618 | BF068898 | 501 | 113 | 166 | 68 | 1e−62 | DNA methylation |
| 2315 | 2.706172777 | up | 0.0000669 | 0.002172462 | AW350048 | 819 | 116 | 177 | 65 | 4e−56 | ribosomal protein S6 |
| 2316 | 2.707140679 | up | 0.00000573 | 0.000820095 | BI975038 | 490 | 55 | 70 | 78 | 3e−24 | |
| 2317 | 2.707576759 | up | 0.000103911 | 0.002697141 | BE555372 | 589 | 47 | 62 | 75 | 3e−18 | |
| 2318 | 2.707898991 | up | 0.001214118 | 0.011703702 | BF069978 | 375 | 16 | 49 | 32 | 2.9 | |
| 2319 | 2.709903037 | up | 0.0000292 | 0.001470471 | AW348250 | 750 | 160 | 186 | 86 | 1e−91 | protein biosynthesis |
| 2320 | 2.710443996 | up | 0.000173462 | 0.003557561 | BE821350 | 204 | 22 | 34 | 64 | 1e−06 | metabolism |
| 2321 | 2.710662906 | up | 0.000242249 | 0.004322552 | CD410890 | 549 | 77 | 81 | 95 | 6e−41 | translational initiation |
| 2322 | 2.711116281 | up | 0.002486094 | 0.018577528 | BG654162 | 193 | 15 | 40 | 37 | 1.9 | |
| 2323 | 2.711431457 | up | 0.000266413 | 0.004557014 | CD402134 | 645 | 97 | 131 | 74 | 3e−54 | protein biosynthesis |
| 2324 | 2.712539292 | up | 0.000527116 | 0.006901124 | BG511649 | 336 | 8 | 13 | 61 | 7.2 | |
| 2325 | 2.712977562 | up | 0.000341958 | 0.005282297 | BE059778 | 429 | 20 | 81 | 24 | 1.4 | electron transport |
| 2326 | 2.714717456 | up | 0.0000557 | 0.001994018 | BG044519 | 417 | 110 | 140 | 78 | 3e−60 | electron transport |
| 2327 | 2.714775578 | up | 0.000032 | 0.001530061 | BF425961 | 478 | 66 | 83 | 79 | 1e−32 | protein biosynthesis |
| 2328 | 2.715399897 | up | 0.000141085 | 0.003154302 | BG239432 | 409 | 60 | 117 | 51 | 2e−24 | cell cycle |
| 2329 | 2.716730289 | up | 0.0000627 | 0.002105997 | CF922241 | 665 | 70 | 89 | 78 | 3e−38 | |
| 2330 | 2.716770765 | up | 0.000717108 | 0.008434426 | BE440278 | 485 | 96 | 134 | 71 | 1e−51 | |
| 2331 | 2.717636875 | up | 0.000139991 | 0.00314554 | BE473577 | 490 | 16 | 58 | 27 | 1.4 | DNA replication |
| 2332 | 2.718348007 | up | 0.000882583 | 0.009575935 | CF806688 | 716 | 185 | 217 | 85 | e−109 | |
| 2333 | 2.720625687 | up | 0.000489466 | 0.006605946 | AW423975 | | | | | | |
| 2334 | 2.721834741 | up | 0.0000452 | 0.001793484 | BI945113 | 213 | 12 | 41 | 29 | 5.5 | ribosomal protein S13, tRNA |
| | | | | | | | | | | | aminoacylation for protein translation |
| 2335 | 2.721871632 | up | 0.0000679 | 0.002183171 | BG651387 | 347 | 63 | 74 | 85 | 8e−33 | protein biosynthesis |
| 2336 | 2.7222617 | up | 0.000464136 | 0.006385522 | BM567825 | 288 | 17 | 61 | 27 | 1.8 | |
| 2337 | 2.723278818 | up | 0.0000603 | 0.002066371 | CA853274 | 351 | 11 | 25 | 44 | 1.4 | electron transport |
| 2338 | 2.723925312 | up | 0.000104573 | 0.002705473 | CF808180 | 713 | 143 | 171 | 83 | 3e−81 | Vacuolar processing enzyme 2 |
| | | | | | | | | | | | (VPE2), protein catabolism |
| 2339 | 2.725544839 | up | 0.003174186 | 0.021783259 | AW394550 | 437 | 34 | 51 | 66 | 1e−15 | |
| 2340 | 2.726282456 | up | 0.00006 | 0.002064083 | CD418513 | 683 | 23 | 70 | 32 | 1e−05 | |
| 2341 | 2.727065926 | up | 0.000124288 | 0.002953231 | CD413372 | 565 | 20 | 20 | 100 | 3e−05 | protein targeting |
| 2342 | 2.727898066 | up | 0.0000933 | 0.002553736 | BU925879 | 624 | 118 | 215 | 54 | 4e−53 | microtubule−based movement |
| 2343 | 2.729093795 | up | 0.0000741 | 0.00228806 | BI969927 | 696 | 114 | 150 | 76 | 1e−57 | nucleosome assembly |
| 2344 | 2.731368659 | up | 0.000284419 | 0.004739979 | AW424057 | 274 | 13 | 25 | 52 | 3.2 | protein amino acid phosphorylation |
| 2345 | 2.733130133 | up | 0.00017776 | 0.00361419 | CD404912 | 710 | 60 | 92 | 65 | 3e−25 | ubiquitin-dependent protein |
| | | | | | | | | | | | catabolism |
| 2346 | 2.734765547 | up | 0.000191249 | 0.003740861 | CF807720 | 622 | 137 | 158 | 86 | 8e−75 | |
| 2347 | 2.734772909 | up | 0.000238304 | 0.004286736 | BG406239 | 393 | 18 | 50 | 36 | 1.2 | |
| 2348 | 2.734776112 | up | 0.000233164 | 0.004234772 | BG509216 | 411 | 10 | 13 | 76 | 4.9 | |
| 2349 | 2.735400794 | up | 0.00000388 | 0.000767688 | BU544110 | 512 | 63 | 99 | 63 | 6e−30 | |
| 2350 | 2.736025137 | up | 0.000105243 | 0.00270768 | CD395158 | 672 | 106 | 122 | 86 | 3e−55 | protein biosynthesis |
| 2351 | 2.736257774 | up | 0.00039737 | 0.005783094 | AF007100.1 | | | | | | biotin carboxylase precursor |
| 2352 | 2.736455534 | up | 0.0000969 | 0.00260019 | CD397204 | 654 | 131 | 165 | 79 | 2e−73 | N-terminal protein amino acid |
| | | | | | | | | | | | acetylation |
| 2353 | 2.736587424 | up | 0.0000718 | 0.002248657 | CD392833 | 560 | 42 | 85 | 49 | 3e−13 | |
| 2354 | 2.737209962 | up | 0.000042 | 0.001750979 | BU761975 | 484 | 88 | 116 | 75 | 4e−52 | electron transport |
| 2355 | 2.738131803 | up | 0.000132697 | 0.003052755 | CD401340 | 680 | 120 | 140 | 85 | 2e−65 | protein biosynthesis |
| 2356 | 2.742661671 | up | 0.0000542 | 0.001976427 | BI970273 | 719 | 135 | 188 | 71 | 1e−70 | RNA processing |
| 2357 | 2.743451712 | up | 0.000322569 | 0.005109176 | CD390967 | 689 | 101 | 153 | 66 | 1e−61 | carbohydrate metabolism |
| 2358 | 2.746158814 | up | 0.0000927 | 0.00255292 | CD413539 | 564 | 76 | 101 | 75 | 2e−35 | protein biosynthesis |
| 2359 | 2.748015992 | up | 0.0000193 | 0.001235676 | CA785676 | 559 | 105 | 166 | 63 | 5e−59 | electron transport |
| 2360 | 2.749199969 | up | 0.00000123 | 0.000704945 | AW734044 | 457 | 42 | 104 | 40 | 4e−13 | |
| 2361 | 2.751917003 | up | 0.000361661 | 0.005454491 | AW350801 | 696 | 118 | 157 | 75 | 1e−64 | |
| 2362 | 2.752390899 | up | 0.003059772 | 0.021261824 | CF805702 | 545 | 97 | 178 | 54 | 2e−53 | |
| 2363 | 2.753364627 | up | 0.00000886 | 0.000915272 | CD399794 | 581 | 84 | 119 | 70 | 4e−43 | |
| 2364 | 2.755505506 | up | 0.000172137 | 0.003538112 | BI788080 | 376 | 11 | 43 | 25 | 2.2 | |
| 2365 | 2.755514336 | up | 0.00000596 | 0.000830526 | CD414830 | 327 | 10 | 21 | 47 | 2.4 | cell wall biosynthesis (sensu |
| | | | | | | | | | | | Magnoliophyta) |
| 2366 | 2.755772629 | up | 0.000302776 | 0.004906774 | BM523157 | 504 | 34 | 68 | 50 | 6e−11 | |
| 2367 | 2.757981302 | up | 0.00011803 | 0.002882263 | BI701724 | 562 | 137 | 164 | 83 | 5e−79 | response to wounding |
| 2368 | 2.758515912 | up | 0.0000183 | 0.00122288 | CF807256 | 577 | 145 | 192 | 75 | 3e−84 | defense response |
| 2369 | 2.759270947 | up | 0.000176012 | 0.003591302 | CD403449 | 675 | 122 | 143 | 85 | 3e−64 | protein biosynthesis |
| 2370 | 2.759374063 | up | 0.000133265 | 0.003062083 | AW733473 | 421 | 18 | 63 | 28 | 0.60 | ciliary/flagellar motility |
| 2371 | 2.759799237 | up | 0.001226584 | 0.011763568 | BU546685 | 384 | 44 | 59 | 74 | 8e−20 | potassium ion transport |
| 2372 | 2.760477196 | up | 0.004790078 | 0.028730408 | BE820574 | 455 | 40 | 59 | 67 | 8e−19 | |
| 2373 | 2.761225486 | up | 0.0000342 | 0.00156033 | BE440442 | 545 | 27 | 42 | 64 | 2e−09 | cell recognition |
| 2374 | 2.761977522 | up | 0.0000827 | 0.002431497 | CD409801 | 518 | 21 | 21 | 100 | 8e−06 | protein targeting |
| 2375 | 2.762190071 | up | 0.00000746 | 0.000889839 | CD399794 | 581 | 84 | 119 | 70 | 4e−43 | |
| 2376 | 2.762603721 | up | 0.0000401 | 0.001711701 | CD416891 | 586 | 67 | 99 | 67 | 5e−36 | |
| 2377 | 2.76295428 | up | 0.0000394 | 0.001702189 | BM094307 | 550 | 95 | 171 | 55 | 2e−49 | |
| 2378 | 2.764012135 | up | 0.00000373 | 0.000764834 | AW318170 | 592 | 132 | 167 | 79 | 3e−74 | protein biosynthesis |
| 2379 | 2.764614562 | up | 0.000171012 | 0.003526557 | CD402576 | 546 | 44 | 55 | 80 | 9e−21 | |
| 2380 | 2.76474267 | up | 0.001379392 | 0.012660918 | CA851281 | 653 | 153 | 203 | 75 | 5e−87 | GMP biosynthesis |
| 2381 | 2.764968253 | up | 0.000225311 | 0.004139945 | CD393403 | 627 | 58 | 118 | 49 | 4e−25 | |
| 2382 | 2.765170097 | up | 0.000351522 | 0.005368458 | BI969339 | 493 | 63 | 71 | 88 | 1e−34 | transcription |
| 2383 | 2.765369646 | up | 0.0000509 | 0.001916523 | BE659595 | 628 | 84 | 93 | 90 | 3e−46 | protein biosynthesis |
| 2384 | 2.765422213 | up | 0.000461281 | 0.006362533 | BU546201 | 569 | 31 | 39 | 79 | 6e−12 | protein folding |
| 2385 | 2.768727885 | up | 0.00000762 | 0.000892439 | AF243360.1 | | | | | | glutathione S-transferase GST 5 |
| 2386 | 2.768909914 | up | 0.0000294 | 0.0014708 | CD401125 | 558 | 15 | 37 | 40 | 1.1 | |
| 2387 | 2.769647796 | up | 0.0000599 | 0.002064083 | CF807857 | 614 | 162 | 183 | 88 | 5e−94 | |
| 2388 | 2.769688843 | up | 0.00000796 | 0.000899116 | BG362878 | 580 | 121 | 177 | 68 | 2e−73 | |
| 2389 | 2.770452455 | up | 0.000193528 | 0.003767789 | CD408785 | 470 | 52 | 61 | 85 | 2e−26 | |
| 2390 | 2.772779352 | up | 0.0000326 | 0.001543897 | AW396169 | 430 | 10 | 27 | 37 | 7.0 | multidrug transport |
| 2391 | 2.773394502 | up | 0.000186958 | 0.003701183 | BF596892 | 397 | 29 | 37 | 78 | 2e−11 | |
| 2392 | 2.774991827 | up | 0.0000197 | 0.00125198 | CF807089 | 444 | 48 | 81 | 59 | 2e−21 | |
| 2393 | 2.775057034 | up | 0.0000309 | 0.001500454 | BF597733 | 541 | 144 | 174 | 82 | 2e−82 | tricarboxylic acid cycle |
| 2394 | 2.775425508 | up | 0.0000657 | 0.002159139 | CD405520 | 275 | 14 | 48 | 29 | 2.4 | ribosomal protein L2 |
| 2395 | 2.776622438 | up | 0.0000267 | 0.001397859 | CF808630 | 393 | 88 | 103 | 85 | 4e−47 | adenosine salvage pathway |
| 2396 | 2.777034913 | up | 0.0000397 | 0.001711389 | BE611803 | 556 | 166 | 180 | 92 | 3e−90 | proline biosynthesis |
| 2397 | 2.778271315 | up | 0.000369775 | 0.005508397 | CD398024 | 664 | 95 | 169 | 56 | 8e−42 | |
| 2398 | 2.778558417 | up | 0.0000452 | 0.001793484 | CF807383 | 460 | 96 | 108 | 88 | 6e−51 | electron transport |
| 2399 | 2.778577596 | up | 0.000533643 | 0.006950291 | BM886770 | 538 | 133 | 155 | 85 | 9e−74 | Proteasome IOTA subunit |
| 2400 | 2.779647826 | up | 0.000108679 | 0.002771436 | CD392275 | 721 | 148 | 160 | 92 | 6e−81 | |
| 2401 | 2.780739861 | up | 0.000146657 | 0.003216456 | CD401876 | 455 | 24 | 59 | 40 | 4e−06 | |
| 2402 | 2.781033957 | up | 0.000252526 | 0.004420006 | AF236108.1 | | | | | | |
| 2403 | 2.781302014 | up | 0.0000245 | 0.001353718 | CD409912 | 678 | 116 | 154 | 75 | 7e−62 | |
| 2404 | 2.783234735 | up | 0.00000994 | 0.000951145 | BQ786271 | 592 | 51 | 65 | 78 | 3e−23 | mitochondrial translocation |
| 2405 | 2.784006754 | up | 0.001713262 | 0.014543584 | CD400361 | 683 | 108 | 129 | 83 | 6e−56 | |
| 2406 | 2.785812852 | up | 0.0000296 | 0.00147316 | BE022395 | 303 | 22 | 25 | 88 | 1e−07 | protein biosynthesis |
| 2407 | 2.786084001 | up | 0.000042 | 0.001750979 | CD397099 | 678 | 100 | 128 | 78 | 1e−53 | mitochondrial translocation |
| 2408 | 2.786149019 | up | 0.0000057 | 0.000820095 | BE610582 | 391 | 52 | 86 | 60 | 4e−23 | DNA replication |
| 2409 | 2.788156535 | up | 0.000189849 | 0.003732626 | CD410040 | 536 | 84 | 125 | 67 | 1e−42 | electron transport |
| 2410 | 2.788650008 | up | 0.0000675 | 0.002177084 | BG045204 | 541 | 54 | 145 | 37 | 4e−24 | |
| 2411 | 2.788902894 | up | 0.001492245 | 0.013294481 | BM143646 | 593 | 93 | 149 | 62 | 2e−49 | metabolism |
| 2412 | 2.789372343 | up | 0.0000134 | 0.001091338 | BF424576 | 516 | 114 | 140 | 81 | 6e−59 | |
| 2413 | 2.790209547 | up | 0.0000705 | 0.002227455 | BU550728 | 651 | 97 | 155 | 62 | 2e−52 | protein biosynthesis |
| 2414 | 2.790274037 | up | 0.00000365 | 0.000764834 | CF805929 | 481 | 60 | 105 | 57 | 9e−29 | |
| 2415 | 2.790838745 | up | 0.000100081 | 0.002647286 | BE583765 | 559 | 68 | 92 | 73 | 5e−31 | response to heat |
| 2416 | 2.792747926 | up | 0.000167282 | 0.003479645 | AW423661 | 492 | 65 | 86 | 75 | 3e−38 | |
| 2417 | 2.793399103 | up | 0.0000109 | 0.000983942 | AW348883 | 613 | 15 | 41 | 36 | 0.024 | |
| 2418 | 2.793960483 | up | 0.0000496 | 0.001884481 | CD400835 | 650 | 96 | 113 | 84 | 1e−54 | translational elongation |
| 2419 | 2.797596154 | up | 0.001063468 | 0.010817995 | BF595743 | 667 | 47 | 98 | 47 | 3e−12 | |
| 2420 | 2.79816901 | up | 0.001024495 | 0.010575012 | CD406784 | 662 | 50 | 75 | 66 | 4e−25 | |
| 2421 | 2.801204738 | up | 0.000313806 | 0.005022772 | BG237453 | 257 | 10 | 17 | 58 | 4.2 | lipid metabolism |
| 2422 | 2.803533387 | up | 0.000132466 | 0.003049321 | BU550191 | 560 | 83 | 111 | 74 | 2e−44 | histidine biosynthesis |
| 2423 | 2.807182052 | up | 0.001599323 | 0.013896265 | BM732317 | 565 | 62 | 106 | 58 | 2e−31 | |
| 2424 | 2.807255467 | up | 0.0000438 | 0.001772209 | CD398369 | 647 | 71 | 107 | 66 | 2e−34 | |
| 2425 | 2.807885643 | up | 0.003902592 | 0.024935249 | CD392933 | 564 | 36 | 57 | 63 | 9e−17 | |
| 2426 | 2.807938348 | up | 0.0000048 | 0.000799682 | CF807811 | 701 | 99 | 144 | 68 | 9e−49 | metabolism |
| 2427 | 2.808824003 | up | 0.000423484 | 0.006008648 | BU082274 | 582 | 114 | 174 | 65 | 3e−60 | nicotinamide adenine dinucleotide |
| | | | | | | | | | | | biosynthesis |
| 2428 | 2.810645203 | up | 0.000625165 | 0.00774538 | AW780481 | 346 | 17 | 51 | 33 | 2.5 | metabolism |
| 2429 | 2.81166011 | up | 0.000062 | 0.002096544 | AW277783 | 434 | 95 | 102 | 93 | 6e−50 | transcription |
| 2430 | 2.812091204 | up | 0.0000116 | 0.001020193 | BQ786370 | 591 | 134 | 201 | 66 | 3e−69 | glycolysis |
| 2431 | 2.812144972 | up | 0.00000384 | 0.000764834 | BU545884 | 594 | 101 | 140 | 72 | 2e−55 | |
| 2432 | 2.812530073 | up | 0.000680665 | 0.008140714 | AI856233 | 740 | 92 | 109 | 84 | 2e−47 | proline biosynthesis |
| 2433 | 2.815192048 | up | 0.000118083 | 0.002882263 | BE820998 | 780 | 139 | 177 | 78 | 5e−80 | pyrimidine base metabolism |
| 2434 | 2.815415419 | up | 0.0000142 | 0.001112836 | BU549516 | 417 | 20 | 44 | 45 | 0.005 | |
| 2435 | 2.816721939 | up | 0.004514593 | 0.027613937 | CF809112 | 373 | 38 | 44 | 86 | 1e−18 | histidine biosynthesis |
| 2436 | 2.817894714 | up | 0.0000291 | 0.001466884 | BI785798 | 542 | 13 | 39 | 33 | 3.0 | |
| 2437 | 2.818081214 | up | 0.00000915 | 0.000924406 | BI969821 | 717 | 127 | 178 | 71 | 5e−67 | electron transport |
| 2438 | 2.818269095 | up | 0.000124083 | 0.002952452 | CA783137 | 785 | 221 | 261 | 84 | e−125 | |
| 2439 | 2.820412521 | up | 0.00024448 | 0.004343819 | CF805765 | 551 | 137 | 140 | 97 | 3e−76 | |
| 2440 | 2.821314695 | up | 0.00000927 | 0.000924406 | CK605695 | 843 | 157 | 231 | 67 | 2e−85 | protein biosynthesis |
| 2441 | 2.822861704 | up | 0.0000894 | 0.002514157 | AW471967 | 250 | 17 | 48 | 35 | 2.4 | |
| 2442 | 2.823122095 | up | 0.00084484 | 0.009327209 | BI974314 | 560 | 113 | 140 | 80 | 3e−63 | cysteine biosynthesis from serine |
| 2443 | 2.823510889 | up | 0.0000686 | 0.002184143 | CD403005 | 460 | 20 | 67 | 29 | 2.2 | protein amino acid phosphorylation |
| 2444 | 2.823773164 | up | 0.0000102 | 0.000961453 | AI496072 | 283 | 15 | 42 | 35 | 1.4 | regulation of transcription\, DNA- |
| | | | | | | | | | | | dependent |
| 2445 | 2.824986708 | up | 0.00010036 | 0.002652799 | CF807643 | 742 | 234 | 247 | 94 | e−134 | |
| 2446 | 2.82629353 | up | 0.001417431 | 0.012856283 | CD395739 | 520 | 17 | 32 | 53 | 0.005 | |
| 2447 | 2.826326602 | up | 0.0000312 | 0.001505932 | BM306984 | 413 | 21 | 29 | 72 | 2e−06 | DNA metabolism |
| 2448 | 2.827067472 | up | 0.00012604 | 0.002974387 | CD392610 | 583 | 46 | 51 | 90 | 4e−23 | |
| 2449 | 2.827769192 | up | 0.000405975 | 0.005858599 | CF806742 | 499 | 53 | 57 | 92 | 2e−26 | |
| 2450 | 2.827974841 | up | 0.00141845 | 0.012856283 | BE822726 | 439 | 17 | 54 | 31 | 0.036 | |
| 2451 | 2.82852499 | up | 0.000102549 | 0.002675093 | BU549086 | 621 | 122 | 161 | 75 | 2e−75 | response to UV |
| 2452 | 2.828719804 | up | 0.0000922 | 0.002547532 | CD417931 | 677 | 106 | 131 | 80 | 5e−50 | microtubule-based movement |
| 2453 | 2.828813195 | up | 0.000112466 | 0.002812701 | AW350618 | 636 | 111 | 135 | 82 | 1e−56 | |
| 2454 | 2.829899212 | up | 0.0000175 | 0.001205969 | AW309774 | 369 | 13 | 35 | 37 | 2.8 | metabolism |
| 2455 | 2.832261313 | up | 0.00000524 | 0.000809562 | BQ473336 | 277 | 12 | 19 | 63 | 3.2 | |
| 2456 | 2.834033273 | up | 0.0000829 | 0.002431824 | BQ253259 | 255 | 11 | 34 | 32 | 9.5 | regulation of transcription |
| 2457 | 2.835916113 | up | 0.0000517 | 0.001934676 | BE209656 | 479 | 50 | 62 | 80 | 6e−23 | |
| 2458 | 2.836091445 | up | 0.0000077 | 0.000894724 | CD414300 | 637 | 112 | 134 | 83 | 2e−60 | |
| 2459 | 2.836224918 | up | 0.002315958 | 0.017702181 | CD391457 | 665 | 70 | 112 | 62 | 7e−30 | chloroplast organization and |
| | | | | | | | | | | | biogenesis |
| 2460 | 2.836915549 | up | 0.000136203 | 0.003091825 | BU927244 | 556 | 36 | 72 | 50 | 2e−14 | |
| 2461 | 2.837761838 | up | 0.0000288 | 0.001464287 | BQ273082 | 602 | 136 | 162 | 83 | 2e−76 | |
| 2462 | 2.838522495 | up | 0.0000965 | 0.002595294 | BU544988 | 570 | 70 | 99 | 70 | 1e−35 | metabolism |
| 2463 | 2.839973476 | up | 0.00000728 | 0.000883683 | AI736416 | 405 | 77 | 134 | 57 | 9e−36 | |
| 2464 | 2.841119692 | up | 0.0000849 | 0.002462244 | AW705539 | 399 | 17 | 59 | 28 | 2.0 | |
| 2465 | 2.841694788 | up | 0.0000331 | 0.001543897 | CF806180 | 648 | 74 | 87 | 85 | 6e−39 | |
| 2466 | 2.842727703 | up | 0.0000127 | 0.001070114 | BM092911 | 557 | 67 | 120 | 55 | 6e−29 | fatty acid biosynthesis |
| 2467 | 2.842904656 | up | 0.000209352 | 0.003963047 | AW509412 | 559 | 140 | 187 | 74 | 2e−71 | branched chain family amino acid |
| | | | | | | | | | | | biosynthesis |
| 2468 | 2.844889258 | up | 0.000129985 | 0.003029272 | AI440945 | 361 | 103 | 120 | 85 | 2e−58 | |
| 2469 | 2.845646617 | up | 0.0000933 | 0.002553736 | BU549072 | 379 | 11 | 32 | 34 | 0.27 | |
| 2470 | 2.846553489 | up | 0.000005 | 0.000799682 | BF596398 | 481 | 66 | 93 | 70 | 5e−32 | asparagine biosynthesis |
| 2471 | 2.848011285 | up | 0.0000462 | 0.001810721 | CD402259 | 623 | 52 | 90 | 57 | 1e−26 | |
| 2472 | 2.848593676 | up | 0.0000091 | 0.000924406 | BM178738 | 544 | 155 | 181 | 85 | 4e−84 | |
| 2473 | 2.848667493 | up | 0.000169783 | 0.003507803 | BI945128 | 315 | 15 | 43 | 34 | 1.1 | electron transport |
| 2474 | 2.848998879 | up | 0.0000255 | 0.001374317 | BU550101 | 353 | 12 | 18 | 66 | 0.13 | |
| 2475 | 2.849338286 | up | 0.0000437 | 0.001771311 | AW348250 | 750 | 160 | 186 | 86 | 1e−91 | protein biosynthesis |
| 2476 | 2.849871987 | up | 0.000115363 | 0.002848472 | CD403513 | 678 | 112 | 147 | 76 | 6e−63 | |
| 2477 | 2.85018327 | up | 0.00041919 | 0.005972503 | CF808206 | 568 | 103 | 128 | 80 | 5e−59 | |
| 2478 | 2.850531466 | up | 0.00000313 | 0.000720842 | BU551102 | 632 | 53 | 77 | 68 | 2e−26 | |
| 2479 | 2.851950008 | up | 0.000384696 | 0.005647919 | CD392889 | 646 | 82 | 95 | 86 | 1e−45 | protein biosynthesis |
| 2480 | 2.854262602 | up | 0.0000034 | 0.000750585 | CF805829 | 380 | 18 | 36 | 50 | 6e−06 | transport |
| 2481 | 2.855421379 | up | 0.0000962 | 0.002593544 | BE022033 | 433 | 111 | 124 | 89 | 7e−61 | |
| 2482 | 2.856537382 | up | 0.000526927 | 0.00690105 | CD400862 | 698 | 137 | 157 | 87 | 3e−75 | glycine metabolism |
| 2483 | 2.857562963 | up | 0.000138428 | 0.003129107 | BG238929 | 475 | 109 | 126 | 86 | 2e−57 | |
| 2484 | 2.857728078 | up | 0.00000251 | 0.000712455 | AB061212.1 | | | | | | flavonoid 3′-hydroxylase |
| 2485 | 2.857967812 | up | 0.000115858 | 0.002848619 | CF805755 | 563 | 118 | 166 | 71 | 5e−60 | |
| 2486 | 2.858148649 | up | 0.003194485 | 0.021855919 | BE330075 | 497 | 12 | 32 | 37 | 0.52 | metabolism |
| 2487 | 2.86477516 | up | 0.000216448 | 0.004052504 | BM731847 | 557 | 169 | 185 | 91 | e−100 | protein biosynthesis |
| 2488 | 2.866584872 | up | 0.000826284 | 0.009184346 | BE346365 | 584 | 117 | 198 | 59 | 3e−48 | thylakoid membrane organization |
| | | | | | | | | | | | and biogenesis |
| 2489 | 2.866664618 | up | 0.0000399 | 0.001711701 | BI945664 | 643 | 14 | 32 | 43 | 3.1 | protein amino acid prenylation |
| 2490 | 2.867488531 | up | 0.001777614 | 0.014934817 | BF067399 | 520 | 117 | 147 | 79 | 1e−62 | protein folding |
| 2491 | 2.867846409 | up | 0.0000177 | 0.00121239 | AW349709 | 745 | 111 | 209 | 53 | 1e−51 | rRNA modification |
| 2492 | 2.869563063 | up | 0.0000504 | 0.001904034 | BU548485 | 618 | 90 | 124 | 72 | 6e−48 | proteolysis and peptidolysis |
| 2493 | 2.871149732 | up | 0.000111071 | 0.002790978 | CD403449 | 675 | 122 | 143 | 85 | 3e−64 | protein biosynthesis |
| 2494 | 2.872679474 | up | 0.003621373 | 0.023722455 | CD418280 | 710 | 90 | 139 | 64 | 1e−48 | |
| 2495 | 2.87433946 | up | 0.002816248 | 0.020162347 | BQ628677 | 384 | 8 | 22 | 36 | 9.1 | metabolism |
| 2496 | 2.875125489 | up | 0.0000998 | 0.002646119 | BQ785932 | 420 | 36 | 61 | 59 | 1e−13 | |
| 2497 | 2.877392778 | up | 0.000755266 | 0.008716958 | BI321171 | | | | | | |
| 2498 | 2.878680599 | up | 0.000011 | 0.000991166 | BI969403 | 303 | 16 | 54 | 29 | 1.1 | |
| 2499 | 2.87882866 | up | 0.0000271 | 0.001410713 | BI322110 | 491 | 43 | 62 | 69 | 2e−17 | |
| 2500 | 2.879486381 | up | 0.000051 | 0.001917433 | BM270192 | 317 | 13 | 29 | 44 | 2.4 | dicarboxylic acid transport |
| 2501 | 2.880369074 | up | 0.000039 | 0.001690358 | BI471550 | 433 | 43 | 80 | 53 | 7e−14 | |
| 2502 | 2.881084263 | up | 0.0000649 | 0.002146014 | CD398452 | 508 | 84 | 90 | 93 | 2e−44 | |
| 2503 | 2.885448859 | up | 0.000191629 | 0.003744376 | BU762116 | 421 | 16 | 17 | 94 | 0.011 | |
| 2504 | 2.885803415 | up | 0.000179205 | 0.003632031 | BE820450 | 207 | 18 | 23 | 78 | 1e−04 | |
| 2505 | 2.885878086 | up | 0.0000175 | 0.001205969 | BI970823 | 650 | 74 | 111 | 66 | 1e−36 | |
| 2506 | 2.887865039 | up | 0.0000924 | 0.002551441 | BG510363 | 491 | 42 | 50 | 84 | 1e−16 | |
| 2507 | 2.888507501 | up | 0.000343093 | 0.00529332 | AF007100.1 | | | | | | biotin carboxylase precursor |
| 2508 | 2.889627599 | up | 0.0000219 | 0.001310321 | BI317274 | 508 | 107 | 170 | 62 | 1e−58 | proline dehydrogenase, glutamate |
| | | | | | | | | | | | biosynthesis |
| 2509 | 2.890489552 | up | 0.000222337 | 0.004111572 | CA935387 | 419 | 26 | 30 | 86 | 9e−10 | carbohydrate metabolism |
| 2510 | 2.891072278 | up | 0.00000999 | 0.000951145 | AW100408 | 447 | 27 | 33 | 81 | 8e−10 | nucleosome assembly |
| 2511 | 2.891370332 | up | 0.000268697 | 0.004584151 | BM893125 | 557 | 101 | 110 | 91 | 2e−54 | |
| 2512 | 2.892319422 | up | 0.000168285 | 0.003491387 | AW705534 | 286 | 10 | 14 | 71 | 5.4 | malate dehydrogenase |
| 2513 | 2.89339001 | up | 0.000159084 | 0.003365449 | CD398650 | 721 | 54 | 69 | 78 | 1e−28 | |
| 2514 | 2.894026147 | up | 0.0000163 | 0.001156628 | BQ094646 | 499 | 111 | 168 | 66 | 3e−54 | protein biosynthesis |
| 2515 | 2.894064133 | up | 0.0000433 | 0.001770252 | CD391431 | 677 | 89 | 146 | 60 | 2e−44 | |
| 2516 | 2.895030247 | up | 0.00000565 | 0.000820095 | AW733613 | 269 | 15 | 34 | 44 | 2.5 | protein amino acid phosphorylation |
| 2517 | 2.896493491 | up | 0.000144079 | 0.003182607 | BE823322 | 637 | 63 | 113 | 55 | 3e−28 | |
| 2518 | 2.8969555 | up | 0.001333137 | 0.012402821 | BM309390 | 596 | 161 | 203 | 79 | 6e−90 | |
| 2519 | 2.897770098 | up | 0.00591744 | 0.033171176 | BM731979 | 521 | 61 | 155 | 39 | 7e−33 | cellulose biosynthesis |
| 2520 | 2.89967791 | up | 0.000178974 | 0.003632031 | CF809073 | 468 | 140 | 155 | 90 | 6e−81 | |
| 2521 | 2.900423081 | up | 0.000157492 | 0.003347279 | CD404099 | 691 | 83 | 99 | 83 | 3e−43 | glycolysis |
| 2522 | 2.900555198 | up | 0.0000854 | 0.002466308 | BI970873 | 759 | 143 | 191 | 74 | 2e−81 | |
| 2523 | 2.902157034 | up | 0.002006735 | 0.01616995 | BU546254 | 598 | 71 | 106 | 66 | 6e−40 | |
| 2524 | 2.904886535 | up | 0.000135786 | 0.003091672 | BQ612756 | 429 | 58 | 75 | 77 | 3e−28 | fatty acid biosynthesis |
| 2525 | 2.905991557 | up | 0.00000531 | 0.000810852 | CF806357 | 712 | 127 | 220 | 57 | 1e−65 | protein biosynthesis |
| 2526 | 2.906336752 | up | 0.00000296 | 0.000720842 | BE609695 | 569 | 97 | 186 | 52 | 1e−56 | anion transport |
| 2527 | 2.906847085 | up | 0.000140445 | 0.003147284 | CA935124 | 575 | 69 | 86 | 80 | 8e−37 | protein biosynthesis |
| 2528 | 2.907001146 | up | 0.00020163 | 0.003868666 | AW596159 | 392 | 38 | 58 | 65 | 1e−17 | |
| 2529 | 2.907158029 | up | 0.0000226 | 0.001321932 | CD391778 | 711 | 65 | 96 | 67 | 1e−32 | |
| 2530 | 2.907664939 | up | 0.00000215 | 0.000707618 | CD399594 | 487 | 27 | 35 | 77 | 3e−08 | |
| 2531 | 2.912036992 | up | 0.0000398 | 0.001711403 | CF808605 | 708 | 157 | 219 | 71 | 3e−90 | |
| 2532 | 2.913413108 | up | 0.000765623 | 0.00878528 | BF067399 | 520 | 117 | 147 | 79 | 1e−62 | protein folding |
| 2533 | 2.917662596 | up | 0.00000166 | 0.000704945 | AW704524 | 434 | 81 | 142 | 57 | 5e−41 | |
| 2534 | 2.918248154 | up | 0.000357189 | 0.005421838 | CF807032 | 479 | 67 | 141 | 47 | 2e−24 | ossification |
| 2535 | 2.918979551 | up | 0.0000482 | 0.001849638 | CF806157 | 630 | 97 | 112 | 86 | 1e−53 | |
| 2536 | 2.919724837 | up | 0.000407927 | 0.005867922 | AI966408 | 265 | 13 | 28 | 46 | 2.5 | |
| 2537 | 2.920026134 | up | 0.00016302 | 0.003424662 | CF806202 | 623 | 104 | 130 | 80 | 3e−52 | |
| 2538 | 2.922965743 | up | 0.00000303 | 0.000720842 | CA801700 | 488 | 32 | 40 | 80 | 1e−12 | translational elongation |
| 2539 | 2.923400628 | up | 0.0000436 | 0.001771311 | BI969813 | 758 | 76 | 131 | 58 | 2e−37 | |
| 2540 | 2.924109933 | up | 0.000304834 | 0.004923155 | CD402878 | 620 | 136 | 139 | 97 | 1e−75 | |
| 2541 | 2.925617891 | up | 0.000184896 | 0.003691451 | BG651980 | 522 | 78 | 91 | 85 | 5e−43 | mRNA splicing |
| 2542 | 2.926950025 | up | 0.001662604 | 0.014254613 | CA802847 | 272 | 8 | 18 | 44 | 5.5 | regulation of transcription\, DNA- |
| | | | | | | | | | | | dependent |
| 2543 | 2.933517307 | up | 0.000170173 | 0.003513114 | CD406986 | 687 | 57 | 191 | 29 | 6e−16 | |
| 2544 | 2.933606227 | up | 0.0000686 | 0.002184143 | AF532625.1 | | | | | | GTP-binding protein |
| 2545 | 2.935424278 | up | 0.00012471 | 0.002961387 | BM177889 | 542 | 71 | 130 | 54 | 2e−33 | |
| 2546 | 2.936572434 | up | 0.0000548 | 0.001984819 | BE824394 | 739 | 148 | 208 | 71 | 5e−81 | proteolysis and peptidolysis |
| 2547 | 2.936623144 | up | 0.000015 | 0.001128257 | AW704532 | 517 | 118 | 169 | 69 | 3e−62 | |
| 2548 | 2.937136118 | up | 0.0000799 | 0.002385734 | BF068240 | 375 | 26 | 46 | 56 | 4e−09 | fatty acid biosynthesis |
| 2549 | 2.937661141 | up | 0.0000101 | 0.000954671 | CD407203 | 689 | 17 | 25 | 68 | 0.006 | |
| 2550 | 2.938436791 | up | 0.000298985 | 0.004867734 | BU546621 | 684 | 40 | 71 | 56 | 2e−17 | |
| 2551 | 2.939782285 | up | 0.001038505 | 0.010664401 | BE822726 | 439 | 17 | 54 | 31 | 0.036 | |
| 2552 | 2.939806775 | up | 0.0000247 | 0.001361395 | CD411279 | 673 | 90 | 123 | 73 | 3e−51 | |
| 2553 | 2.941977946 | up | 0.00013872 | 0.003129806 | BM093897 | | | | | | Ribosomal protein L37 (RPL37) |
| 2554 | 2.942075797 | up | 0.0000227 | 0.001321932 | BE657385 | 658 | 26 | 100 | 26 | 0.85 | regulation of transcription\, DNA- |
| | | | | | | | | | | | dependent |
| 2555 | 2.944025236 | up | 0.0000166 | 0.00116924 | CD405886 | 671 | 86 | 123 | 69 | 1e−46 | |
| 2556 | 2.945627886 | up | 0.00000798 | 0.000899116 | CF807829 | 553 | 128 | 132 | 96 | 3e−69 | translational initiation |
| 2557 | 2.946291352 | up | 0.00000802 | 0.000899116 | BU545889 | 631 | 129 | 164 | 78 | 2e−67 | protein biosynthesis |
| 2558 | 2.94663734 | up | 0.0000474 | 0.001827727 | CD403414 | 521 | 61 | 86 | 70 | 2e−32 | |
| 2559 | 2.946668588 | up | 0.000552486 | 0.007106409 | CD414740 | 434 | 17 | 50 | 34 | 0.86 | metabolism |
| 2560 | 2.94762141 | up | 0.00083749 | 0.009267833 | BG881834 | 374 | 20 | 34 | 58 | 3e−04 | |
| 2561 | 2.948297747 | up | 0.0000727 | 0.002264259 | BU577650 | 286 | 13 | 33 | 39 | 1.4 | |
| 2562 | 2.949453169 | up | 0.0000331 | 0.001543897 | CD412544 | 665 | 92 | 110 | 83 | 3e−50 | |
| 2563 | 2.950417279 | up | 0.0000294 | 0.0014708 | L14930.1 | | | | | | (Rab7p) |
| 2564 | 2.95146501 | up | 0.0000792 | 0.002374851 | BE824353 | 607 | 85 | 91 | 93 | 2e−45 | protein biosynthesis |
| 2565 | 2.95412662 | up | 0.00000216 | 0.000707618 | BG047187 | 500 | 97 | 134 | 72 | 2e−49 | |
| 2566 | 2.954681313 | up | 0.000209972 | 0.003970778 | BF324501 | 477 | 68 | 95 | 71 | 5e−28 | protein biosynthesis |
| 2567 | 2.954820068 | up | 0.0000359 | 0.001602355 | CF809287 | 534 | 64 | 71 | 90 | 3e−30 | 40S ribosomal protein S25 |
| 2568 | 2.955146308 | up | 0.000352441 | 0.005371525 | BI425242 | 519 | 51 | 64 | 79 | 3e−23 | amino acid transport |
| 2569 | 2.955734276 | up | 0.00000797 | 0.000899116 | CD396245 | 614 | 106 | 125 | 84 | 1e−56 | protein biosynthesis |
| 2570 | 2.960582686 | up | 0.00000411 | 0.000768376 | CD403849 | 473 | 17 | 38 | 44 | 0.79 | |
| 2571 | 2.961695317 | up | 0.001100825 | 0.011027016 | AI794662 | 325 | 16 | 45 | 35 | 1.9 | |
| 2572 | 2.964718501 | up | 0.001849175 | 0.015271019 | CF808206 | 568 | 103 | 128 | 80 | 5e−59 | |
| 2573 | 2.967922249 | up | 0.00026737 | 0.004569214 | BU764037 | 620 | 133 | 154 | 86 | 3e−75 | response to wounding |
| 2574 | 2.968330678 | up | 0.000133821 | 0.003068701 | CD415745 | 480 | 19 | 23 | 82 | 8e−06 | |
| 2575 | 2.971907046 | up | 0.000196481 | 0.003801663 | BI967715 | 360 | 16 | 41 | 39 | 0.021 | cell growth and/or maintenance |
| 2576 | 2.973498974 | up | 0.000589092 | 0.007433112 | BG651827 | 500 | 32 | 76 | 42 | 1e−11 | regulation of transcription |
| 2577 | 2.975165928 | up | 0.0000148 | 0.001127466 | CK606384 | 885 | 124 | 198 | 62 | 9e−56 | ossification |
| 2578 | 2.97542537 | up | 0.000798296 | 0.008982983 | AF329816.1 | | | | | | Cu/Zn-superoxide dismutase copper |
| | | | | | | | | | | | chaperone precursor |
| 2579 | 2.979477604 | up | 0.000163066 | 0.003424662 | BG362769 | 500 | 34 | 55 | 61 | 1e−13 | |
| 2580 | 2.980887166 | up | 0.000143598 | 0.003181853 | AW349716 | 707 | 48 | 64 | 75 | 7e−20 | CBP protein |
| 2581 | 2.982185608 | up | 0.000115194 | 0.002846846 | CD415149 | | | | | | |
| 2582 | 2.98611595 | up | 0.0000318 | 0.001527864 | BE610938 | 402 | 22 | 79 | 27 | 1.6 | |
| 2583 | 2.987472084 | up | 0.0000294 | 0.0014708 | CD399557 | 565 | 83 | 92 | 90 | 7e−45 | protein biosynthesis |
| 2584 | 2.98852849 | up | 0.000112557 | 0.00281309 | CD412647 | 670 | 53 | 57 | 92 | 4e−26 | |
| 2585 | 2.988704973 | up | 0.00000267 | 0.000720842 | CD393802 | 634 | 46 | 63 | 73 | 6e−22 | branched chain family amino acid |
| | | | | | | | | | | | biosynthesis |
| 2586 | 2.989246923 | up | 0.000754996 | 0.008716958 | BU545129 | 504 | 36 | 43 | 83 | 1e−15 | regulation of transcription |
| 2587 | 2.989264848 | up | 0.0000583 | 0.00202784 | BE820846 | 410 | 37 | 40 | 92 | 2e−15 | |
| 2588 | 2.991188951 | up | 0.000474918 | 0.006481706 | BU544815 | 582 | 66 | 114 | 57 | 3e−29 | |
| 2589 | 2.993058391 | up | 0.000138792 | 0.003129806 | BM523459 | 589 | 44 | 128 | 34 | 4e−17 | |
| 2590 | 2.993338638 | up | 0.0000116 | 0.001020193 | BM270196 | 335 | 18 | 52 | 34 | 1.9 | |
| 2591 | 2.995650426 | up | 0.000103715 | 0.002694163 | CD404738 | 638 | 78 | 107 | 72 | 1e−40 | |
| 2592 | 2.998319091 | up | 0.000539243 | 0.006989346 | BE473714 | 562 | 120 | 190 | 63 | 3e−63 | |
| 2593 | 3.0027242 | up | 0.004191492 | 0.026159928 | AI494661 | 412 | 63 | 83 | 75 | 3e−33 | electron transport |
| 2594 | 3.003769648 | up | 0.001132032 | 0.011226013 | BE347902 | 487 | 106 | 161 | 65 | 9e−59 | |
| 2595 | 3.004041412 | up | 0.00000162 | 0.000704945 | BE823151 | 571 | 33 | 88 | 37 | 4e−05 | |
| 2596 | 3.004706468 | up | 0.00028079 | 0.004710812 | CD399549 | 671 | 84 | 128 | 65 | 9e−46 | protein amino acid phosphorylation |
| 2597 | 3.006242639 | up | 0.0000274 | 0.001421826 | BE823533 | 608 | 75 | 89 | 84 | 3e−38 | regulation of transcription\, DNA- |
| | | | | | | | | | | | dependent |
| 2598 | 3.011539131 | up | 0.0000563 | 0.002005588 | BE658001 | 267 | 10 | 23 | 43 | 7.2 | |
| 2599 | 3.013804932 | up | 0.000435305 | 0.006112439 | BU090070 | 553 | 35 | 63 | 55 | 6e−12 | protein biosynthesis |
| 2600 | 3.014221023 | up | 0.000115411 | 0.002848472 | CD404759 | 589 | 17 | 62 | 27 | 0.54 | regulation of transcription\, DNA- |
| | | | | | | | | | | | dependent |
| 2601 | 3.016262481 | up | 0.000195352 | 0.003789559 | BE347663 | 379 | 14 | 22 | 63 | 0.032 | |
| 2602 | 3.019326079 | up | 0.0000239 | 0.00133979 | BE657555 | 613 | 71 | 122 | 58 | 1e−29 | fatty acid biosynthesis |
| 2603 | 3.0210722 | up | 0.00000915 | 0.000924406 | BU926440 | 586 | 106 | 182 | 58 | 1e−57 | |
| 2604 | 3.021776172 | up | 0.0000571 | 0.002012438 | BI968156 | 791 | 42 | 68 | 61 | 5e−17 | translational elongation |
| 2605 | 3.022622639 | up | 0.000117815 | 0.002881326 | CD396718 | 418 | 36 | 81 | 44 | 9e−10 | |
| 2606 | 3.02459908 | up | 0.0000288 | 0.001464287 | BU084372 | 420 | 63 | 77 | 81 | 1e−30 | protein biosynthesis |
| 2607 | 3.02550285 | up | 0.0000774 | 0.002341624 | BM954626 | 558 | 144 | 186 | 77 | 2e−84 | carbohydrate metabolism |
| 2608 | 3.026364336 | up | 0.0000655 | 0.002157866 | BU965056 | 630 | 167 | 186 | 89 | 1e−96 | translational elongation |
| 2609 | 3.029250829 | up | 0.0000663 | 0.002164602 | BI699799 | 523 | 14 | 45 | 31 | 1.6 | |
| 2610 | 3.031631262 | up | 0.0000686 | 0.002184143 | BG238743 | 441 | 18 | 48 | 37 | 4.4 | |
| 2611 | 3.032067914 | up | 0.0000183 | 0.00122288 | AW756908 | 368 | 16 | 47 | 34 | 0.44 | regulation of transcription |
| 2612 | 3.03314249 | up | 0.0000081 | 0.000899886 | AW317212 | 486 | 27 | 55 | 49 | 2e−10 | mitochondrial translocation |
| 2613 | 3.033666379 | up | 0.0000532 | 0.001958687 | BI969619 | 771 | 151 | 183 | 82 | 4e−91 | |
| 2614 | 3.034098509 | up | 0.00000887 | 0.000915272 | CA938497 | 530 | 106 | 120 | 88 | 6e−59 | |
| 2615 | 3.035187414 | up | 0.0000225 | 0.001321932 | CD400631 | 409 | 61 | 95 | 64 | 3e−31 | transport |
| 2616 | 3.037260736 | up | 0.00000253 | 0.000712455 | BM085372 | 533 | 54 | 87 | 62 | 5e−29 | |
| 2617 | 3.041962505 | up | 0.0000368 | 0.001628134 | CD395921 | 440 | 39 | 46 | 84 | 8e−18 | |
| 2618 | 3.042690842 | up | 0.0000139 | 0.001103908 | CF806665 | 636 | 57 | 100 | 57 | 2e−27 | |
| 2619 | 3.043076748 | up | 0.00000534 | 0.000810852 | CD415935 | 391 | 15 | 46 | 32 | 3.3 | |
| 2620 | 3.043240161 | up | 0.00000482 | 0.000799682 | BE330529 | 611 | 128 | 159 | 80 | 1e−74 | |
| 2621 | 3.043521743 | up | 0.001548551 | 0.013612339 | CD416698 | 594 | 22 | 67 | 32 | 0.003 | |
| 2622 | 3.045385153 | up | 0.0000939 | 0.00255704 | CA803049 | 351 | 16 | 44 | 36 | 0.17 | protein amino acid phosphorylation |
| 2623 | 3.046577233 | up | 0.000110927 | 0.002790978 | BU927487 | 592 | 67 | 97 | 69 | 2e−34 | potassium ion transport |
| 2624 | 3.047169893 | up | 0.0000633 | 0.002118926 | AW101182 | 329 | 11 | 28 | 39 | 5.6 | |
| 2625 | 3.047491679 | up | 0.00000737 | 0.000884012 | CD401299 | 696 | 99 | 113 | 87 | 2e−54 | protein biosynthesis |
| 2626 | 3.047991174 | up | 0.00000189 | 0.000704945 | CK606431 | 532 | 59 | 86 | 68 | 1e−29 | protein biosynthesis |
| 2627 | 3.05042148 | up | 0.00000517 | 0.000803843 | BU549161 | 574 | 58 | 95 | 61 | 6e−26 | |
| 2628 | 3.051032573 | up | 0.00000919 | 0.000924406 | BE023126 | 428 | 8 | 16 | 50 | 1.9 | |
| 2629 | 3.051698403 | up | 0.0000231 | 0.001328603 | AW349133 | 664 | 63 | 126 | 50 | 6e−24 | |
| 2630 | 3.052483267 | up | 0.0000042 | 0.00077152 | BM308645 | 393 | 10 | 35 | 28 | 4.4 | protein amino acid phosphorylation |
| 2631 | 3.052661444 | up | 0.0000948 | 0.002566638 | CD392230 | 635 | 66 | 90 | 73 | 4e−33 | |
| 2632 | 3.053173873 | up | 0.000219033 | 0.004072519 | CF808422 | 150 | 33 | 37 | 89 | 9e−14 | |
| 2633 | 3.053234422 | up | 0.0000148 | 0.001127466 | AI443369 | 642 | 86 | 113 | 76 | 8e−47 | |
| 2634 | 3.056355442 | up | 0.000228068 | 0.00416636 | BE820998 | 780 | 139 | 177 | 78 | 5e−80 | pyrimidine base metabolism |
| 2635 | 3.057649341 | up | 0.0000132 | 0.001086969 | BM885090 | 530 | 149 | 175 | 85 | 2e−84 | lysine biosynthesis via |
| | | | | | | | | | | | diaminopimelate |
| 2636 | 3.058572271 | up | 0.0000228 | 0.001321932 | CD391486 | 698 | 104 | 144 | 72 | 1e−56 | |
| 2637 | 3.058578547 | up | 0.00000795 | 0.000899116 | BU927016 | 606 | 140 | 181 | 77 | 5e−80 | cytochrome biogenesis |
| 2638 | 3.059203164 | up | 0.000164308 | 0.00344113 | BU765089 | 408 | 46 | 109 | 42 | 3e−16 | |
| 2639 | 3.059779255 | up | 0.0000797 | 0.002383605 | CD393958 | 655 | 121 | 153 | 79 | 2e−66 | |
| 2640 | 3.060048065 | up | 0.004625362 | 0.028042933 | CD397627 | 700 | 121 | 152 | 79 | 3e−67 | response to hydrogen peroxide |
| 2641 | 3.062073107 | up | 0.00000815 | 0.000899886 | BU548960 | 594 | 97 | 112 | 86 | 6e−54 | electron transport |
| 2642 | 3.066711956 | up | 0.000609202 | 0.007607741 | CF806157 | 630 | 97 | 112 | 86 | 1e−53 | |
| 2643 | 3.069102445 | up | 0.000034 | 0.001558741 | CD408617 | 654 | 70 | 77 | 90 | 2e−36 | |
| 2644 | 3.069323989 | up | 0.0000623 | 0.002103106 | CD418243 | 411 | 50 | 67 | 74 | 4e−26 | |
| 2645 | 3.070606177 | up | 0.00000428 | 0.000773405 | BU547003 | 654 | 123 | 170 | 72 | 1e−59 | protein catabolism |
| 2646 | 3.071292916 | up | 0.0000349 | 0.001577606 | BG157835 | | | | | | |
| 2647 | 3.07436449 | up | 0.00000294 | 0.000720842 | AW570423 | 493 | 61 | 93 | 65 | 2e−27 | |
| 2648 | 3.075552736 | up | 0.0000814 | 0.002411249 | CF805660 | 531 | 138 | 164 | 84 | 3e−77 | |
| 2649 | 3.077759768 | up | 0.000185893 | 0.003694813 | BE190740 | 358 | 11 | 21 | 52 | 1.1 | regulation of transcription\, DNA- |
| | | | | | | | | | | | dependent |
| 2650 | 3.078003859 | up | 0.00000726 | 0.000883683 | BG238118 | 502 | 72 | 109 | 66 | 2e−37 | |
| 2651 | 3.080201501 | up | 0.000245103 | 0.00435284 | BE657452 | 779 | 146 | 218 | 66 | 2e−81 | |
| 2652 | 3.080599211 | up | 0.00000432 | 0.000776405 | CD390470 | 607 | 14 | 32 | 43 | 0.014 | G-protein coupled receptor protein |
| | | | | | | | | | | | signaling pathway |
| 2653 | 3.081056304 | up | 0.000408614 | 0.005867922 | BU550637 | 394 | 14 | 49 | 28 | 4.4 | |
| 2654 | 3.082224227 | up | 0.000215132 | 0.004029862 | BM178462 | 560 | 109 | 179 | 60 | 2e−59 | |
| 2655 | 3.083189803 | up | 0.000286304 | 0.004760849 | CF808379 | 421 | 121 | 130 | 93 | 6e−63 | |
| 2656 | 3.086579383 | up | 0.00000825 | 0.000900892 | BU549986 | 621 | 77 | 149 | 51 | 2e−39 | |
| 2657 | 3.086648854 | up | 0.0000286 | 0.001462856 | AW350841 | 527 | 81 | 92 | 88 | 2e−43 | |
| 2658 | 3.087920965 | up | 0.000496607 | 0.006647466 | BU549381 | 582 | 76 | 112 | 67 | 3e−40 | electron transport |
| 2659 | 3.088173124 | up | 0.000340167 | 0.005277719 | BG154978 | 257 | 12 | 31 | 38 | 3.2 | |
| 2660 | 3.090774026 | up | 0.000574171 | 0.007300835 | BI322039 | 422 | 12 | 31 | 38 | 1.4 | |
| 2661 | 3.091365756 | up | 0.00000184 | 0.000704945 | BI975097 | 568 | 143 | 166 | 86 | 4e−80 | selenocysteine incorporation |
| 2662 | 3.097232469 | up | 0.000064 | 0.002134923 | CD398971 | 632 | 69 | 89 | 77 | 9e−37 | |
| 2663 | 3.098816716 | up | 0.00000667 | 0.000860462 | BM178073 | 561 | 81 | 168 | 48 | 2e−33 | ossification |
| 2664 | 3.099994949 | up | 0.00000689 | 0.000870238 | BU927439 | 600 | 124 | 188 | 65 | 1e−71 | |
| 2665 | 3.100011093 | up | 0.00159008 | 0.01385436 | BM525663 | 495 | 74 | 96 | 77 | 1e−40 | translational initiation |
| 2666 | 3.102971704 | up | 0.0000363 | 0.001614615 | BG511667 | 322 | 13 | 29 | 44 | 0.64 | |
| 2667 | 3.103515489 | up | 0.00000525 | 0.000809562 | BE821233 | 669 | 37 | 114 | 32 | 3e−08 | |
| 2668 | 3.104751522 | up | 0.0000354 | 0.001589423 | BQ272942 | 519 | 15 | 43 | 34 | 2.8 | |
| 2669 | 3.105258524 | up | 0.00027285 | 0.004627125 | AI748648 | 794 | 102 | 178 | 57 | 2e−48 | Acidic ribosomal protein P0, protein |
| | | | | | | | | | | | biosynthesis |
| 2670 | 3.108261092 | up | 0.007711779 | 0.03951501 | BE658596 | 655 | 18 | 42 | 42 | 0.13 | G-protein coupled receptor protein |
| | | | | | | | | | | | signaling pathway |
| 2671 | 3.109657668 | up | 0.001369795 | 0.012617052 | BE803165 | 656 | 117 | 147 | 79 | 3e−64 | histidine biosynthesis |
| 2672 | 3.109827972 | up | 0.000381326 | 0.005622468 | CA935572 | 360 | 9 | 26 | 34 | 4.4 | regulation of transcription |
| 2673 | 3.111611194 | up | 0.00000457 | 0.00078556 | BI470477 | 511 | 18 | 55 | 32 | 5.9 | negative regulation of flower |
| | | | | | | | | | | | development |
| 2674 | 3.113228763 | up | 0.0000103 | 0.000962144 | BE059135 | 292 | 13 | 25 | 52 | 1.8 | electron transport |
| 2675 | 3.113798869 | up | 0.0000111 | 0.000992148 | BQ628423 | 208 | 9 | 24 | 37 | 5.6 | |
| 2676 | 3.115286855 | up | 0.000042 | 0.001750979 | CD397537 | 662 | 64 | 104 | 61 | 3e−29 | |
| 2677 | 3.117481342 | up | 0.0000628 | 0.002108954 | CD391486 | 698 | 104 | 144 | 72 | 1e−56 | |
| 2678 | 3.117504788 | up | 0.0000593 | 0.002053168 | BE823847 | 726 | 111 | 141 | 78 | 6e−62 | ‘de novo’ pyrimidine base |
| | | | | | | | | | | | biosynthesis |
| 2679 | 3.120265988 | up | 0.0000194 | 0.001235676 | CF806007 | 662 | 178 | 202 | 88 | 3e−99 | |
| 2680 | 3.122956889 | up | 0.000105506 | 0.00271073 | CD392889 | 646 | 82 | 95 | 86 | 1e−45 | protein biosynthesis |
| 2681 | 3.123328904 | up | 0.001817241 | 0.015125851 | CD417082 | 618 | 44 | 145 | 30 | 0.053 | |
| 2682 | 3.126347821 | up | 0.0000124 | 0.001058667 | BM092393 | 349 | 9 | 19 | 47 | 4.2 | |
| 2683 | 3.128343648 | up | 0.00000934 | 0.000924406 | BI969773 | 752 | 134 | 182 | 73 | 8e−76 | |
| 2684 | 3.130345371 | up | 0.0000107 | 0.000976675 | AW781427 | 368 | 20 | 65 | 30 | 0.26 | |
| 2685 | 3.131443453 | up | 0.00000345 | 0.000753007 | AW568239 | 422 | 102 | 139 | 73 | 6e−57 | electron transport |
| 2686 | 3.132810333 | up | 0.0000607 | 0.002075795 | CF805655 | 501 | 65 | 69 | 94 | 2e−32 | protein biosynthesis |
| 2687 | 3.134182166 | up | 0.00000591 | 0.000830526 | BE823239 | 645 | 73 | 119 | 61 | 5e−38 | metabolism |
| 2688 | 3.136231512 | up | 0.000130435 | 0.00303449 | AW396684 | 622 | 49 | 173 | 28 | 3e−12 | fatty acid biosynthesis |
| 2689 | 3.137199604 | up | 0.000146739 | 0.003216456 | AW349966 | 568 | 89 | 104 | 85 | 9e−46 | |
| 2690 | 3.142413562 | up | 0.0000622 | 0.002101134 | AW101190 | 337 | 17 | 20 | 85 | 3e−05 | regulation of transcription\, DNA- |
| | | | | | | | | | | | dependent |
| 2691 | 3.147240131 | up | 0.0000409 | 0.001731672 | BE822841 | 698 | 114 | 138 | 82 | 6e−56 | |
| 2692 | 3.148315634 | up | 0.00000383 | 0.000764834 | BE659410 | 256 | 10 | 21 | 47 | 1.5 | |
| 2693 | 3.149276604 | up | 0.0000255 | 0.001374317 | AW102527 | 236 | 10 | 22 | 45 | 4.2 | trichome differentiation (sensu |
| | | | | | | | | | | | Magnoliophyta) |
| 2694 | 3.152799752 | up | 0.0000918 | 0.00254498 | BM891517 | 428 | 16 | 20 | 80 | 5e−04 | nucleosome assembly |
| 2695 | 3.153036068 | up | 0.002186045 | 0.017127947 | BF070320 | 639 | 77 | 122 | 63 | 3e−47 | |
| 2696 | 3.155684256 | up | 0.000605021 | 0.00757604 | CD406357 | 668 | 75 | 107 | 70 | 2e−40 | nicotinamide adenine dinucleotide |
| | | | | | | | | | | | biosynthesis |
| 2697 | 3.15594628 | up | 0.00000395 | 0.000767688 | BI971321 | 800 | 111 | 137 | 81 | 2e−63 | spermidine biosynthesis |
| 2698 | 3.156821547 | up | 0.00000856 | 0.000900892 | BU546533 | 571 | 26 | 48 | 54 | 1e−11 | DNA topological change |
| 2699 | 3.156982788 | up | 0.0000189 | 0.001231955 | CD394365 | 369 | 26 | 29 | 89 | 2e−09 | protein biosynthesis |
| 2700 | 3.16095699 | up | 0.0000994 | 0.002639612 | AW472061 | 272 | 14 | 50 | 28 | 4.2 | RNA dependent DNA replication |
| 2701 | 3.164864137 | up | 0.0000788 | 0.002366298 | CD392230 | 635 | 66 | 90 | 73 | 4e−33 | |
| 2702 | 3.166342107 | up | 0.000339665 | 0.005277719 | BE820625 | 737 | 162 | 192 | 84 | 3e−92 | nucleotide-sugar metabolism |
| 2703 | 3.170399867 | up | 0.000945985 | 0.009989411 | CA801508 | 699 | 167 | 226 | 73 | 3e−90 | acetyl-CoA metabolism |
| 2704 | 3.175880867 | up | 0.0000147 | 0.001127441 | CF805937 | 704 | 207 | 232 | 89 | e−124 | asparagine biosynthesis |
| 2705 | 3.176080756 | up | 0.0000282 | 0.001446674 | BE823296 | 525 | 37 | 81 | 45 | 2e−13 | |
| 2706 | 3.177815463 | up | 0.000130693 | 0.00303449 | AI437633 | 641 | 41 | 86 | 47 | 7e−13 | microtubule-based process |
| 2707 | 3.178239467 | up | 0.0000764 | 0.00232891 | CD396136 | 469 | 65 | 69 | 94 | 2e−32 | protein biosynthesis |
| 2708 | 3.179166961 | up | 0.00000554 | 0.00082005 | BE819863 | 539 | 76 | 116 | 65 | 1e−41 | |
| 2709 | 3.179296443 | up | 0.0000332 | 0.001543897 | BG155518 | 254 | 16 | 19 | 84 | 8e−04 | fatty acid biosynthesis |
| 2710 | 3.181257335 | up | 0.00000229 | 0.000707618 | BU548220 | 668 | 52 | 83 | 62 | 4e−24 | |
| 2711 | 3.183077176 | up | 0.0000886 | 0.002506437 | CF807720 | 622 | 137 | 158 | 86 | 8e−75 | |
| 2712 | 3.183227204 | up | 0.0000206 | 0.001272227 | BQ296568 | 588 | 134 | 182 | 73 | 5e−65 | |
| 2713 | 3.183272681 | up | 0.0000049 | 0.000799682 | BU549715 | 511 | 85 | 103 | 82 | 4e−44 | |
| 2714 | 3.184297828 | up | 0.000385744 | 0.005658884 | BF324091 | 563 | 95 | 175 | 54 | 8e−52 | metabolism |
| 2715 | 3.186791024 | up | 0.000230426 | 0.004195181 | BU545941 | 422 | 10 | 20 | 50 | 5.2 | |
| 2716 | 3.187961842 | up | 0.0000237 | 0.00133979 | AW317519 | 595 | 106 | 122 | 86 | 5e−55 | protein biosynthesis |
| 2717 | 3.188507279 | up | 0.00036461 | 0.005474833 | BE022964 | 381 | 10 | 24 | 41 | 3.1 | glycolysis |
| 2718 | 3.189935863 | up | 0.000159262 | 0.003367322 | BE058960 | 185 | 10 | 19 | 52 | 4.2 | |
| 2719 | 3.190005325 | up | 0.000174753 | 0.003574311 | AW348942 | 737 | 120 | 150 | 80 | 4e−60 | protein biosynthesis |
| 2720 | 3.19216179 | up | 0.0000354 | 0.001589423 | CD404926 | 606 | 97 | 112 | 86 | 1e−53 | protein biosynthesis |
| 2721 | 3.193288351 | up | 0.0000465 | 0.001813342 | BI320490 | 549 | 145 | 182 | 79 | 5e−82 | galactose metabolism |
| 2722 | 3.193603467 | up | 0.0000855 | 0.002466308 | BE659156 | 545 | 28 | 54 | 51 | 3e−12 | |
| 2723 | 3.19789894 | up | 0.00000199 | 0.000707618 | BM309598 | 432 | 54 | 65 | 83 | 8e−25 | |
| 2724 | 3.198381188 | up | 0.00000424 | 0.000772527 | BU964565 | 581 | 98 | 135 | 72 | 4e−48 | proteolysis and peptidolysis |
| 2725 | 3.198484582 | up | 0.0000711 | 0.002241459 | BU764193 | 334 | 27 | 55 | 49 | 2e−09 | |
| 2726 | 3.198543866 | up | 0.0000125 | 0.001058667 | BU548826 | 654 | 16 | 59 | 27 | 2.5 | carbohydrate metabolism |
| 2727 | 3.198836035 | up | 0.0000194 | 0.001235676 | AW278139 | 292 | 10 | 24 | 41 | 0.63 | regulation of transcription |
| 2728 | 3.200404911 | up | 0.0000401 | 0.001711701 | CF805655 | 501 | 65 | 69 | 94 | 2e−32 | protein biosynthesis |
| 2729 | 3.202931412 | up | 0.0000583 | 0.00202784 | CK606408 | 739 | 152 | 174 | 87 | 2e−84 | protein biosynthesis |
| 2730 | 3.20294868 | up | 0.0000339 | 0.001558741 | CD412540 | 612 | 104 | 113 | 92 | 8e−56 | |
| 2731 | 3.203344266 | up | 0.0000551 | 0.001987749 | CF806563 | 700 | 171 | 216 | 79 | 7e−94 | protein biosynthesis |
| 2732 | 3.203374443 | up | 0.0000447 | 0.001789988 | CD395628 | 528 | 63 | 77 | 81 | 1e−31 | response to wounding |
| 2733 | 3.203407758 | up | 0.001921536 | 0.015671434 | BQ454087 | 420 | 46 | 60 | 76 | 3e−22 | |
| 2734 | 3.204725055 | up | 0.000042 | 0.001750979 | BE658249 | 594 | 28 | 51 | 54 | 1e−06 | |
| 2735 | 3.205689333 | up | 0.0000294 | 0.0014708 | AI855913 | 471 | 22 | 65 | 33 | 0.055 | |
| 2736 | 3.207108883 | up | 0.00115795 | 0.011388265 | BE820934 | 765 | 94 | 123 | 76 | 4e−50 | acetyl-CoA biosynthesis |
| 2737 | 3.208714089 | up | 0.00000965 | 0.000938105 | CD487471 | 579 | 146 | 180 | 81 | 2e−80 | |
| 2738 | 3.209848386 | up | 0.0000171 | 0.001188863 | CD401340 | 680 | 120 | 140 | 85 | 2e−65 | protein biosynthesis |
| 2739 | 3.216356427 | up | 0.0000118 | 0.001026214 | BF071576 | 305 | 85 | 98 | 86 | 8e−47 | |
| 2740 | 3.216669419 | up | 0.0000124 | 0.001058667 | BM732780 | 579 | 44 | 48 | 91 | 1e−18 | ribosomal protein S13, cytokinesis by |
| | | | | | | | | | | | cell plate formation |
| 2741 | 3.21855933 | up | 0.00000454 | 0.000784876 | CA802301 | 683 | 207 | 220 | 94 | e−118 | |
| 2742 | 3.222544544 | up | 0.00000314 | 0.000720842 | CA938587 | 330 | 23 | 33 | 69 | 4e−07 | |
| 2743 | 3.223187824 | up | 0.00000728 | 0.000883683 | BM092427 | 355 | 20 | 59 | 33 | 0.66 | DNA metabolism |
| 2744 | 3.224766208 | up | 0.002589971 | 0.019075618 | BM091753 | 563 | 77 | 82 | 93 | 1e−40 | |
| 2745 | 3.226973643 | up | 0.0000164 | 0.001160462 | CF808130 | 669 | 184 | 214 | 85 | e−110 | G beta-like protein |
| 2746 | 3.227118536 | up | 0.000865545 | 0.009446444 | CD409767 | 274 | 32 | 85 | 37 | 1e−06 | regulation of transcription |
| 2747 | 3.228048634 | up | 0.000124893 | 0.002962079 | CD391547 | 677 | 102 | 112 | 91 | 2e−57 | protein biosynthesis |
| 2748 | 3.229296232 | up | 0.0000463 | 0.001810721 | BQ253343 | 583 | 159 | 180 | 88 | 8e−90 | pyruvate kinase, glycolysis |
| 2749 | 3.229707094 | up | 0.001624035 | 0.014026743 | BI316840 | 379 | 60 | 126 | 47 | 9e−32 | metabolism |
| 2750 | 3.234853435 | up | 0.00000571 | 0.000820095 | BE210539 | 202 | 13 | 23 | 56 | 2.4 | transport |
| 2751 | 3.235889729 | up | 0.0000194 | 0.001235676 | BQ611537 | 351 | 32 | 33 | 96 | 3e−13 | |
| 2752 | 3.236400928 | up | 0.002853212 | 0.020341304 | AW203386 | 546 | 80 | 167 | 47 | 2e−36 | metabolism |
| 2753 | 3.237530121 | up | 0.002232011 | 0.017328141 | CA799698 | 435 | 108 | 127 | 85 | 1e−64 | |
| 2754 | 3.241090223 | up | 0.000859151 | 0.009416113 | BQ610420 | 187 | 36 | 48 | 75 | 2e−17 | |
| 2755 | 3.241782695 | up | 0.000602129 | 0.007558567 | BI472203 | 495 | 84 | 133 | 63 | 2e−45 | |
| 2756 | 3.244821883 | up | 0.000938146 | 0.009954137 | BF070694 | 362 | 58 | 71 | 81 | 1e−27 | ossification |
| 2757 | 3.246679114 | up | 0.00000153 | 0.000704945 | CA782549 | 430 | 92 | 135 | 68 | 8e−52 | protein complex assembly |
| 2758 | 3.246903218 | up | 0.000126127 | 0.002974387 | BU764231 | 261 | 15 | 43 | 34 | 2.5 | |
| 2759 | 3.2470402 | up | 0.0000494 | 0.001877933 | BI943980 | 377 | 33 | 39 | 84 | 5e−10 | regulation of transcription\, DNA- |
| | | | | | | | | | | | dependent |
| 2760 | 3.248143125 | up | 0.000044 | 0.001775674 | CD396136 | 469 | 65 | 69 | 94 | 2e−32 | protein biosynthesis |
| 2761 | 3.249617028 | up | 0.0000333 | 0.001543897 | CD399552 | 645 | 80 | 146 | 54 | 1e−34 | protein biosynthesis |
| 2762 | 3.253326719 | up | 0.0000142 | 0.001112691 | AW705544 | 390 | 10 | 22 | 45 | 4.3 | |
| 2763 | 3.255700756 | up | 0.000279997 | 0.004703793 | BE821591 | 587 | 81 | 99 | 81 | 2e−43 | GTP biosynthesis |
| 2764 | 3.256810334 | up | 0.0000284 | 0.001453374 | CD415224 | 697 | 113 | 144 | 78 | 1e−60 | |
| 2765 | 3.257592038 | up | 0.0000154 | 0.001135545 | BU760747 | 401 | 91 | 128 | 71 | 8e−50 | tRNA aminoacylation for protein |
| | | | | | | | | | | | translation |
| 2766 | 3.259695832 | up | 0.000314696 | 0.005030588 | AW757263 | 463 | 21 | 63 | 33 | 3.7 | |
| 2767 | 3.260165426 | up | 0.0000647 | 0.002146014 | BM527158 | 558 | 29 | 30 | 96 | 2e−08 | DNA metabolism |
| 2768 | 3.2623341 | up | 0.0000237 | 0.00133979 | AI442855 | 646 | 28 | 37 | 75 | 1e−09 | protein biosynthesis |
| 2769 | 3.26282432 | up | 0.00000173 | 0.000704945 | BE822030 | 590 | 16 | 55 | 29 | 6.0 | protein folding |
| 2770 | 3.263715273 | up | 0.00000126 | 0.000704945 | CF808845 | 688 | 154 | 186 | 82 | 5e−90 | |
| 2771 | 3.266457738 | up | 0.000142529 | 0.003172939 | BI970516 | 792 | 115 | 168 | 68 | 3e−57 | |
| 2772 | 3.266840919 | up | 0.000000994 | 0.000704945 | BQ743039 | 405 | 41 | 47 | 87 | 4e−18 | protein biosynthesis |
| 2773 | 3.268061082 | up | 0.00000145 | 0.000704945 | AW396331 | 306 | 21 | 60 | 35 | 3.2 | |
| 2774 | 3.271092909 | up | 0.001597132 | 0.013880438 | BQ613138 | 275 | 8 | 13 | 61 | 3.2 | |
| 2775 | 3.272302662 | up | 0.0000109 | 0.000982205 | CD409876 | 672 | 14 | 47 | 29 | 0.68 | |
| 2776 | 3.273217587 | up | 0.007181053 | 0.037687756 | BI942102 | 666 | 84 | 95 | 88 | 8e−44 | response to stress |
| 2777 | 3.275939636 | up | 0.00000857 | 0.000900892 | CD403645 | 561 | 16 | 45 | 35 | 1.4 | metal ion transport |
| 2778 | 3.277349406 | up | 0.0000582 | 0.00202784 | CF805809 | 410 | 14 | 55 | 25 | 1.3 | regulation of transcription |
| 2779 | 3.278004411 | up | 0.0000188 | 0.001230468 | CD399268 | 517 | 61 | 68 | 89 | 8e−29 | |
| 2780 | 3.279527277 | up | 0.0000138 | 0.001103908 | AW317168 | 663 | 126 | 143 | 88 | 9e−70 | |
| 2781 | 3.28117725 | up | 0.002378364 | 0.018022249 | CD391589 | 635 | 63 | 124 | 50 | 1e−32 | |
| 2782 | 3.284014987 | up | 0.00000334 | 0.000747115 | AI965477 | 373 | 14 | 45 | 31 | 1.3 | protein amino acid phosphorylation |
| 2783 | 3.284878421 | up | 0.0000189 | 0.001231955 | BE823254 | 636 | 62 | 153 | 40 | 4e−20 | |
| 2784 | 3.285480676 | up | 0.0000552 | 0.001987749 | AW310719 | 515 | 29 | 78 | 37 | 1e−07 | |
| 2785 | 3.287095548 | up | 0.0000744 | 0.002291616 | BE658680 | | | | | | |
| 2786 | 3.29024132 | up | 0.000000515 | 0.00067462 | BI699442 | 540 | 64 | 187 | 34 | 5e−19 | protein folding |
| 2787 | 3.291724509 | up | 0.000204247 | 0.003901743 | AW351074 | 709 | 142 | 202 | 70 | 1e−76 | transport |
| 2788 | 3.293855508 | up | 0.000192646 | 0.00375736 | CD405460 | 533 | 82 | 95 | 86 | 8e−46 | protein biosynthesis |
| 2789 | 3.293917675 | up | 0.003341995 | 0.022544083 | BG511525 | 294 | 20 | 62 | 32 | 3.3 | intracellular protein transport |
| 2790 | 3.294991309 | up | 0.00000755 | 0.000889839 | BI787498 | 600 | 153 | 200 | 76 | 4e−91 | |
| 2791 | 3.297644127 | up | 0.0000979 | 0.002616283 | BE824139 | 527 | 45 | 53 | 84 | 8e−19 | |
| 2792 | 3.298950986 | up | 0.000069 | 0.002191837 | BF596954 | 441 | 12 | 28 | 42 | 2.6 | |
| 2793 | 3.299339007 | up | 0.0000057 | 0.000820095 | BG155432 | 402 | 12 | 32 | 37 | 0.54 | transport |
| 2794 | 3.29944972 | up | 0.001016977 | 0.010511836 | CD396669 | 615 | 50 | 121 | 41 | 8e−22 | |
| 2795 | 3.30129492 | up | 0.0000657 | 0.002159139 | CF809155 | 647 | 113 | 198 | 57 | 2e−62 | purple acid phosphatase |
| 2796 | 3.304000802 | up | 0.0000304 | 0.00148348 | AW317168 | 663 | 126 | 143 | 88 | 9e−70 | similar to XP_533674.1 similar to |
| | | | | | | | | | | | ribosomal protein S16 [Canis |
| | | | | | | | | | | | familiaris] |
| 2797 | 3.304249481 | up | 0.0000857 | 0.002466308 | CD391486 | 698 | 104 | 144 | 72 | 1e−56 | |
| 2798 | 3.305056464 | up | 0.000142103 | 0.003166497 | BF070270 | 353 | 24 | 35 | 68 | 3e−08 | |
| 2799 | 3.305610646 | up | 0.00011334 | 0.002823299 | AW310386 | 573 | 46 | 88 | 52 | 5e−18 | response to wounding |
| 2800 | 3.308978394 | up | 0.0000671 | 0.002177084 | AI442912 | 610 | 85 | 103 | 82 | 2e−44 | protein biosynthesis |
| 2801 | 3.309335884 | up | 0.0000014 | 0.000704945 | CF806279 | 651 | 184 | 214 | 85 | e−104 | |
| 2802 | 3.309772935 | up | 0.000017 | 0.001184989 | CD400116 | 661 | 41 | 56 | 73 | 5e−23 | |
| 2803 | 3.312991114 | up | 0.0000463 | 0.001810721 | BQ611280 | 443 | 17 | 46 | 36 | 4.4 | |
| 2804 | 3.313159016 | up | 0.0000166 | 0.00116924 | CD402482 | 664 | 121 | 134 | 90 | 2e−66 | |
| 2805 | 3.320249177 | up | 0.0000621 | 0.002098737 | CD402664 | 745 | 68 | 88 | 77 | 1e−32 | protein biosynthesis |
| 2806 | 3.321641756 | up | 0.004165328 | 0.026068995 | CD416571 | 596 | 20 | 38 | 52 | 2e−05 | |
| 2807 | 3.324166616 | up | 0.0000208 | 0.001280424 | CD402378 | 614 | 124 | 137 | 90 | 1e−71 | |
| 2808 | 3.326205585 | up | 0.0000192 | 0.001234653 | BU548182 | 588 | 86 | 99 | 86 | 4e−47 | |
| 2809 | 3.329327848 | up | 0.00000243 | 0.000712455 | AW311448 | 289 | 39 | 51 | 76 | 3e−17 | protein biosynthesis |
| 2810 | 3.329612486 | up | 0.00000151 | 0.000704945 | BI785573 | 603 | 104 | 171 | 60 | 1e−58 | |
| 2811 | 3.330087577 | up | 0.000231595 | 0.004212377 | AI437633 | 641 | 41 | 86 | 47 | 7e−13 | microtubule-based process |
| 2812 | 3.333425717 | up | 0.00000256 | 0.000712455 | BE823174 | 478 | 22 | 45 | 48 | 2e−07 | |
| 2813 | 3.334334003 | up | 0.000053 | 0.001956938 | BE209499 | 446 | 51 | 73 | 69 | 6e−23 | protein biosynthesis |
| 2814 | 3.335864864 | up | 0.0000153 | 0.001131323 | CF805772 | 543 | 95 | 141 | 67 | 5e−59 | |
| 2815 | 3.336075657 | up | 0.001268627 | 0.012028774 | CK606389 | 773 | 165 | 224 | 73 | 2e−96 | Ascorbate peroxidase (apx1), |
| | | | | | | | | | | | electron transport |
| 2816 | 3.337779548 | up | 0.00000503 | 0.000799682 | BU083655 | 588 | 49 | 60 | 81 | 3e−21 | protein folding |
| 2817 | 3.33792797 | up | 0.00000627 | 0.000834433 | BG352932 | 504 | 63 | 99 | 63 | 2e−28 | |
| 2818 | 3.338795314 | up | 0.00000788 | 0.000899116 | BU549072 | 379 | 11 | 32 | 34 | 0.27 | |
| 2819 | 3.343538255 | up | 0.001850549 | 0.015274644 | CD390824 | | | | | | |
| 2820 | 3.343913186 | up | 0.0000222 | 0.001314393 | BE657335 | 707 | 12 | 49 | 24 | 1.6 | aromatic amino acid family |
| | | | | | | | | | | | metabolism |
| 2821 | 3.343962154 | up | 0.000777763 | 0.008883548 | CD394954 | 664 | 80 | 125 | 64 | 1e−40 | acetyl-CoA metabolism |
| 2822 | 3.346859599 | up | 0.0000135 | 0.001095315 | AW317691 | 355 | 25 | 30 | 83 | 2e−09 | fatty acid biosynthesis |
| 2823 | 3.347978742 | up | 0.00000279 | 0.000720842 | CD416459 | 563 | 91 | 125 | 72 | 3e−50 | transport |
| 2824 | 3.349177653 | up | 0.000033 | 0.001543897 | CD391234 | 691 | 107 | 123 | 86 | 3e−57 | |
| 2825 | 3.353850316 | up | 0.00000179 | 0.000704945 | CD407646 | 653 | 124 | 147 | 84 | 4e−74 | asparagine biosynthesis |
| 2826 | 3.363802223 | up | 0.00047809 | 0.006499048 | CD392554 | 591 | 29 | 107 | 27 | 0.71 | |
| 2827 | 3.364320418 | up | 0.00000909 | 0.000924406 | CA800410 | 596 | 84 | 158 | 53 | 2e−47 | carbohydrate metabolism |
| 2828 | 3.364439943 | up | 0.0000159 | 0.001150482 | CA801928 | 601 | 66 | 115 | 57 | 6e−27 | |
| 2829 | 3.365514198 | up | 0.00000304 | 0.000720842 | CD397137 | 685 | 71 | 203 | 34 | 5e−29 | |
| 2830 | 3.365890475 | up | 0.0000159 | 0.001150482 | AW756850 | 403 | 63 | 80 | 78 | 2e−33 | tRNA processing |
| 2831 | 3.367685755 | up | 0.00033477 | 0.005235776 | CD415169 | 599 | 58 | 71 | 81 | 5e−27 | |
| 2832 | 3.368644651 | up | 0.0000829 | 0.002431824 | BU082072 | 619 | 27 | 86 | 31 | 3.8 | |
| 2833 | 3.368821471 | up | 0.0000916 | 0.002541909 | BI969363 | 760 | 129 | 149 | 86 | 2e−67 | |
| 2834 | 3.368924216 | up | 0.000028 | 0.001442857 | CD414300 | 637 | 112 | 134 | 83 | 2e−60 | |
| 2835 | 3.3748558 | up | 0.00000497 | 0.000799682 | CA852179 | 234 | 58 | 72 | 80 | 3e−27 | |
| 2836 | 3.375200668 | up | 0.000129786 | 0.003028023 | CF805781 | 552 | 81 | 137 | 59 | 4e−42 | |
| 2837 | 3.376090933 | up | 0.00000862 | 0.000900892 | AF051159.1 | | | | | | ribonucleotide reductase small |
| | | | | | | | | | | | subunit |
| 2838 | 3.376318857 | up | 0.000123642 | 0.002950604 | BU762486 | 389 | 20 | 65 | 30 | 0.39 | |
| 2839 | 3.37670503 | up | 0.00006 | 0.002064083 | AW598259 | 356 | 56 | 75 | 74 | 2e−29 | |
| 2840 | 3.376985258 | up | 0.0000478 | 0.001836359 | BM187772 | 576 | 120 | 137 | 87 | 4e−69 | |
| 2841 | 3.378133057 | up | 0.000352589 | 0.005371525 | CF807504 | 641 | 179 | 212 | 84 | e−101 | glutamate 1-semialdehyde |
| | | | | | | | | | | | aminotransferase |
| 2842 | 3.379404062 | up | 0.0000148 | 0.001127466 | BU577357 | 433 | 55 | 67 | 82 | 8e−25 | |
| 2843 | 3.38000577 | up | 0.0000436 | 0.001771311 | CD398452 | 508 | 84 | 90 | 93 | 2e−44 | |
| 2844 | 3.380042402 | up | 0.000201795 | 0.003868666 | BI786925 | 554 | 153 | 183 | 83 | 4e−85 | DNA metabolism |
| 2845 | 3.381656586 | up | 0.00000547 | 0.000814881 | BG510252 | 400 | 13 | 22 | 59 | 0.41 | protein modification |
| 2846 | 3.381853331 | up | 0.0000414 | 0.001745262 | BG653647 | 349 | 21 | 27 | 77 | 5e−07 | protein biosynthesis |
| 2847 | 3.382289216 | up | 0.0000528 | 0.001953259 | BM891517 | 428 | 16 | 20 | 80 | 5e−04 | nucleosome assembly |
| 2848 | 3.38257736 | up | 0.0000026 | 0.000712455 | CD399870 | 566 | 40 | 58 | 68 | 3e−18 | protein folding |
| 2849 | 3.383621891 | up | 0.00022164 | 0.004101158 | AW760127 | 571 | 113 | 163 | 69 | 6e−56 | protein secretion |
| 2850 | 3.384245101 | up | 0.0000336 | 0.001552651 | BE824191 | 730 | 108 | 152 | 71 | 2e−59 | histidine biosynthesis |
| 2851 | 3.38758616 | up | 0.00000499 | 0.000799682 | AW310167 | 660 | 103 | 128 | 80 | 2e−49 | protein biosynthesis |
| 2852 | 3.393412026 | up | 0.0000314 | 0.001512737 | CD397832 | 465 | 65 | 69 | 94 | 1e−32 | protein biosynthesis |
| 2853 | 3.395273606 | up | 0.000021 | 0.001283285 | AW164185 | 299 | 14 | 34 | 41 | 0.29 | protein biosynthesis |
| 2854 | 3.396892829 | up | 0.000277206 | 0.004677945 | BU550020 | 632 | 92 | 100 | 92 | 3e−49 | purine nucleotide biosynthesis |
| 2855 | 3.398822853 | up | 0.0000062 | 0.000832282 | CD407286 | 397 | 33 | 50 | 66 | 2e−13 | threonine catabolism |
| 2856 | 3.405527215 | up | 0.0000932 | 0.002553736 | AW349147 | 790 | 149 | 197 | 75 | 1e−83 | protein biosynthesis |
| 2857 | 3.406182736 | up | 0.000110472 | 0.002790944 | AW101209 | 430 | 21 | 58 | 36 | 0.49 | G-protein coupled receptor protein |
| | | | | | | | | | | | signaling pathway |
| 2858 | 3.408642365 | up | 0.000277452 | 0.004678935 | BQ298270 | 325 | 17 | 54 | 31 | 1.4 | |
| 2859 | 3.409698074 | up | 0.0000463 | 0.001810721 | CF808861 | 481 | 123 | 150 | 82 | 7e−66 | ubiquitin-dependent protein |
| | | | | | | | | | | | catabolism |
| 2860 | 3.411052129 | up | 0.00000259 | 0.000712455 | BG156909 | 289 | 13 | 43 | 30 | 5.4 | |
| 2861 | 3.417233773 | up | 0.0000054 | 0.000812263 | CA851201 | 442 | 90 | 116 | 77 | 1e−45 | transport |
| 2862 | 3.418011672 | up | 0.000521095 | 0.006853309 | BE821183 | 643 | 31 | 65 | 47 | 4e−11 | |
| 2863 | 3.422220848 | up | 0.00327585 | 0.022257555 | AW423444 | 343 | 13 | 42 | 30 | 1.4 | |
| 2864 | 3.422279604 | up | 0.0000811 | 0.002408976 | BU578999 | 434 | 115 | 134 | 85 | 3e−62 | malate dehydrogenase, tricarboxylic |
| | | | | | | | | | | | acid cycle |
| 2865 | 3.422758454 | up | 0.0000736 | 0.00228073 | BG363198 | 477 | 27 | 44 | 61 | 8e−09 | |
| 2866 | 3.423097407 | up | 0.000733086 | 0.008560791 | CD391181 | 696 | 121 | 124 | 97 | 2e−66 | microtubule-based process |
| 2867 | 3.42771796 | up | 0.0000111 | 0.000993386 | BQ785854 | 611 | 84 | 147 | 57 | 2e−38 | protein folding |
| 2868 | 3.428179006 | up | 0.001173626 | 0.01149304 | AW570625 | 352 | 82 | 117 | 70 | 7e−40 | |
| 2869 | 3.431045525 | up | 0.00000608 | 0.000832282 | AW706620 | 337 | 54 | 68 | 79 | 1e−26 | signal transduction |
| 2870 | 3.431512923 | up | 0.000019 | 0.001231955 | CF806640 | 678 | 165 | 204 | 80 | 2e−94 | protein biosynthesis |
| 2871 | 3.432693628 | up | 0.0000272 | 0.001411089 | CF806861 | 684 | 107 | 191 | 56 | 1e−50 | translational elongation |
| 2872 | 3.433168533 | up | 0.00000821 | 0.000900527 | BE610709 | 412 | 18 | 46 | 39 | 4.9 | proteolysis and peptidolysis |
| 2873 | 3.434523949 | up | 0.00495523 | 0.029389153 | CD397262 | 438 | 38 | 61 | 62 | 2e−14 | metabolism |
| 2874 | 3.436652508 | up | 0.00000602 | 0.000832282 | CD416538 | 381 | 21 | 27 | 77 | 5e−08 | |
| 2875 | 3.436657684 | up | 0.00000761 | 0.000892439 | CD395292 | 664 | 79 | 137 | 57 | 8e−39 | protein biosynthesis |
| 2876 | 3.439944279 | up | 0.0000531 | 0.00195852 | AW704049 | 607 | 25 | 25 | 100 | 5e−07 | |
| 2877 | 3.444477245 | up | 0.000332714 | 0.005214459 | CD394876 | 429 | 33 | 47 | 70 | 6e−15 | regulation of transcription |
| 2878 | 3.445694945 | up | 0.0000172 | 0.001190375 | CA850620 | 274 | 17 | 51 | 33 | 0.13 | |
| 2879 | 3.446258423 | up | 0.000108349 | 0.00276488 | CA785044 | 647 | 20 | 75 | 26 | 3.2 | |
| 2880 | 3.447063366 | up | 0.00000687 | 0.000870238 | CF808696 | 470 | 56 | 86 | 65 | 1e−25 | protein biosynthesis |
| 2881 | 3.448889154 | up | 0.00000311 | 0.000720842 | BU965345 | 565 | 145 | 189 | 76 | 2e−81 | electron transport |
| 2882 | 3.451587468 | up | 0.0000155 | 0.001135545 | CK606099 | 810 | 142 | 172 | 82 | 3e−80 | protein biosynthesis |
| 2883 | 3.452023425 | up | 0.000114499 | 0.002833761 | BQ627548 | 427 | 19 | 54 | 35 | 0.82 | |
| 2884 | 3.454505124 | up | 0.00000365 | 0.000764834 | AW309173 | 330 | 13 | 20 | 65 | 0.23 | protein complex assembly |
| 2885 | 3.458047326 | up | 0.0000101 | 0.000954671 | AF020553.1 | | | | | | ribosome-associated protein p40 |
| 2886 | 3.461005535 | up | 0.000123993 | 0.002952452 | BG881900 | 534 | 142 | 178 | 79 | 6e−86 | proteolysis and peptidolysis |
| 2887 | 3.462045492 | up | 0.0000517 | 0.001934676 | CF806830 | 532 | 89 | 92 | 96 | 7e−50 | |
| 2888 | 3.472568358 | up | 0.00021903 | 0.004072519 | BE022964 | 381 | 10 | 24 | 41 | 3.1 | glycolysis |
| 2889 | 3.475869559 | up | 0.0000242 | 0.001346142 | BE804606 | 413 | 43 | 57 | 75 | 5e−22 | mitochondrial translocation |
| 2890 | 3.475974426 | up | 0.0000301 | 0.001477923 | BI974513 | 530 | 103 | 126 | 81 | 8e−54 | |
| 2891 | 3.47675537 | up | 0.000983036 | 0.010236964 | BF595470 | 448 | 19 | 74 | 25 | 1.2 | |
| 2892 | 3.478377658 | up | 0.00000883 | 0.000915272 | BM893027 | 555 | 122 | 169 | 72 | 3e−63 | electron transport |
| 2893 | 3.481176763 | up | 0.00000138 | 0.000704945 | BU547220 | 620 | 136 | 165 | 82 | 1e−75 | |
| 2894 | 3.485454946 | up | 0.00000491 | 0.000799682 | CF808448 | 484 | 94 | 103 | 91 | 8e−54 | protein biosynthesis |
| 2895 | 3.48613573 | up | 0.000134357 | 0.003074036 | BU547080 | 620 | 55 | 68 | 80 | 5e−29 | response to heat |
| 2896 | 3.488023711 | up | 0.0000227 | 0.001321932 | CK606099 | 810 | 142 | 172 | 82 | 3e−80 | protein biosynthesis |
| 2897 | 3.48813289 | up | 0.000339396 | 0.005277719 | BM095099 | 552 | 59 | 90 | 65 | 3e−26 | |
| 2898 | 3.490456872 | up | 0.00000772 | 0.000894724 | BU761560 | 578 | 144 | 154 | 93 | 8e−77 | |
| 2899 | 3.491855015 | up | 0.00000513 | 0.000800719 | CF806439 | 692 | 98 | 127 | 77 | 3e−56 | |
| 2900 | 3.493289943 | up | 0.0000196 | 0.001246701 | CD416674 | 734 | 132 | 159 | 83 | 4e−73 | ribosomal protein S11 |
| 2901 | 3.495445438 | up | 0.000015 | 0.001128338 | BI426133 | 364 | 49 | 57 | 85 | 2e−23 | |
| 2902 | 3.498877796 | up | 0.00000806 | 0.000899116 | CD400867 | 641 | 55 | 117 | 47 | 1e−22 | protein-mitochondrial targeting |
| 2903 | 3.502827726 | up | 0.0000939 | 0.00255704 | AW596381 | 543 | 58 | 78 | 74 | 6e−30 | |
| 2904 | 3.513307142 | up | 0.000024 | 0.001341839 | CD402482 | 664 | 121 | 134 | 90 | 2e−66 | |
| 2905 | 3.513608427 | up | 0.0000395 | 0.001703138 | AI856076 | 708 | 136 | 153 | 88 | 1e−74 | |
| 2906 | 3.515689626 | up | 0.00000147 | 0.000704945 | AI941451 | 549 | 119 | 180 | 66 | 9e−64 | mitochondrial translocation |
| 2907 | 3.516987861 | up | 0.00000978 | 0.000945362 | CD395105 | 696 | 127 | 154 | 82 | 1e−65 | protein biosynthesis |
| 2908 | 3.51735126 | up | 0.00000941 | 0.000924632 | CA820460 | 567 | 41 | 48 | 85 | 5e−19 | Ribosomal protein L37 (RPL37), |
| | | | | | | | | | | | protein biosynthesis |
| 2909 | 3.518027401 | up | 0.000309695 | 0.004984544 | BI785710 | 454 | 47 | 160 | 29 | 3e−13 | tricarboxylic acid cycle |
| 2910 | 3.519763666 | up | 0.0000777 | 0.002341624 | AI748648 | 794 | 102 | 178 | 57 | 2e−48 | Acidic ribosomal protein P0, protein |
| | | | | | | | | | | | biosynthesis |
| 2911 | 3.521477532 | up | 0.0000297 | 0.00147316 | BU761432 | 590 | 19 | 58 | 32 | 0.92 | oligopeptide transport |
| 2912 | 3.522619992 | up | 0.00000356 | 0.000764744 | CF807389 | 703 | 125 | 184 | 67 | 9e−68 | chloroplast transport |
| 2913 | 3.526090136 | up | 0.0000238 | 0.00133979 | AI736711 | 477 | 42 | 68 | 61 | 3e−18 | regulation of transcription\, DNA- |
| | | | | | | | | | | | dependent |
| 2914 | 3.53343652 | up | 0.0000441 | 0.00177615 | AW350465 | 684 | 114 | 141 | 80 | 7e−65 | protein biosynthesis |
| 2915 | 3.533716292 | up | 0.0000547 | 0.0019813 | BG839241 | 748 | 37 | 70 | 52 | 1e−15 | electron transport |
| 2916 | 3.536552717 | up | 0.002708256 | 0.019650526 | CD395419 | 686 | 42 | 49 | 85 | 1e−19 | protein biosynthesis |
| 2917 | 3.539123415 | up | 0.00000587 | 0.000830526 | BI427211 | 437 | 34 | 34 | 100 | 2e−13 | nucleosome assembly |
| 2918 | 3.544858072 | up | 0.00000609 | 0.000832282 | CD414300 | 637 | 112 | 134 | 83 | 2e−60 | |
| 2919 | 3.54510168 | up | 0.000097 | 0.00260019 | BE822084 | 727 | 129 | 155 | 83 | 1e−69 | response to cold |
| 2920 | 3.551166866 | up | 0.0000569 | 0.002010833 | BQ252711 | 378 | 28 | 32 | 87 | 6e−12 | protein biosynthesis |
| 2921 | 3.552132611 | up | 0.0000348 | 0.001576325 | CD407905 | 607 | 70 | 67 | 80 | 8e−37 | protein biosynthesis |
| 2922 | 3.558140974 | up | 0.0000106 | 0.000974923 | AW351049 | 450 | 37 | 59 | 62 | 3e−16 | |
| 2923 | 3.558615051 | up | 0.0000664 | 0.002164602 | BU548342 | 513 | 15 | 43 | 34 | 1.6 | |
| 2924 | 3.558770687 | up | 0.0000859 | 0.002466308 | CD406256 | 650 | 101 | 112 | 90 | 4e−56 | protein biosynthesis |
| 2925 | 3.561695615 | up | 0.0000105 | 0.00096796 | BQ298286 | 316 | 12 | 18 | 66 | 2.4 | cation transport |
| 2926 | 3.563546895 | up | 0.0000126 | 0.001062091 | CD399552 | 645 | 80 | 146 | 54 | 1e−34 | protein biosynthesis |
| 2927 | 3.568369319 | up | 0.0000137 | 0.001103751 | CA800410 | 596 | 84 | 158 | 53 | 2e−47 | carbohydrate metabolism |
| 2928 | 3.568585276 | up | 0.0000368 | 0.001628134 | BM307231 | 605 | 14 | 44 | 31 | 0.74 | |
| 2929 | 3.572772027 | up | 0.001781917 | 0.014957604 | BE556116 | | | | | | |
| 2930 | 3.574631426 | up | 0.00015744 | 0.003347279 | BI468935 | 548 | 29 | 43 | 67 | 1e−12 | |
| 2931 | 3.576346271 | up | 0.0000649 | 0.002146014 | AW309615 | 657 | 127 | 142 | 89 | 1e−68 | protein biosynthesis |
| 2932 | 3.57653012 | up | 0.0000267 | 0.001397859 | BU548685 | 714 | 153 | 179 | 85 | 5e−88 | protein biosynthesis |
| 2933 | 3.576545285 | up | 0.0000374 | 0.001641557 | CD414411 | 654 | 83 | 119 | 69 | 1e−41 | protein biosynthesis |
| 2934 | 3.57857377 | up | 0.0000373 | 0.001641557 | L28004.1 | | | | | | TGACG-motif-binding factor |
| 2935 | 3.57991353 | up | 0.0000658 | 0.002159139 | CF808962 | 724 | 152 | 191 | 79 | 1e−84 | |
| 2936 | 3.582099715 | up | 0.0000348 | 0.001575241 | CD407905 | 607 | 70 | 87 | 80 | 8e−37 | protein biosynthesis |
| 2937 | 3.583079657 | up | 0.001590648 | 0.013856102 | CD414564 | 309 | 13 | 22 | 59 | 0.84 | carbohydrate metabolism |
| 2938 | 3.583678177 | up | 0.000018 | 0.00121727 | CK605999 | 443 | 51 | 62 | 82 | 5e−23 | |
| 2939 | 3.583847934 | up | 0.0000169 | 0.001184427 | BG839241 | 748 | 37 | 70 | 52 | 1e−15 | Cytochrome P450-like protein, clone |
| | | | | | | | | | | | CP6, electron transport |
| 2940 | 3.585121755 | up | 0.0000152 | 0.00113073 | CF805650 | 488 | 69 | 95 | 72 | 8e−36 | 40S ribosomal S4 protein |
| 2941 | 3.585778412 | up | 0.0000844 | 0.002455821 | BE820846 | 410 | 37 | 40 | 92 | 2e−15 | |
| 2942 | 3.586644237 | up | 0.0000123 | 0.001058667 | AW458157 | 273 | 11 | 32 | 34 | 2.4 | |
| 2943 | 3.589569854 | up | 0.00151789 | 0.013446517 | AW099997 | 424 | 36 | 112 | 32 | 2e−08 | |
| 2944 | 3.596541932 | up | 0.00000476 | 0.000799682 | BE820482 | 693 | 40 | 63 | 63 | 4e−18 | nucleosome assembly |
| 2945 | 3.601234332 | up | 0.0000187 | 0.001230468 | AW350857 | 633 | 86 | 166 | 51 | 2e−47 | carbohydrate metabolism |
| 2946 | 3.605508232 | up | 0.000134262 | 0.003073736 | CD391234 | 691 | 107 | 123 | 86 | 3e−57 | |
| 2947 | 3.606268559 | up | 0.005814139 | 0.032748281 | U12150.1 | | | | | | protease inhibitor |
| 2948 | 3.606718822 | up | 0.0000895 | 0.002514157 | BM308587 | 521 | 88 | 104 | 84 | 3e−47 | mRNA splicing |
| 2949 | 3.613995244 | up | 0.000016 | 0.0011509 | BU964617 | 612 | 160 | 179 | 89 | 6e−86 | |
| 2950 | 3.614290823 | up | 0.00000481 | 0.000799682 | BM527855 | 569 | 61 | 108 | 56 | 3e−28 | |
| 2951 | 3.614409732 | up | 0.0000105 | 0.00096796 | BF596461 | 425 | 88 | 105 | 83 | 2e−45 | protein biosynthesis |
| 2952 | 3.614967076 | up | 0.000126282 | 0.002976179 | AW310566 | | | | | | |
| 2953 | 3.617701592 | up | 0.00002 | 0.001255458 | AW349594 | 581 | 9 | 17 | 52 | 4.5 | electron transport |
| 2954 | 3.617746786 | up | 0.000000437 | 0.00067462 | CD395363 | 665 | 14 | 37 | 37 | 7.4 | |
| 2955 | 3.622190662 | up | 0.000289009 | 0.004776276 | CF809073 | 468 | 140 | 155 | 90 | 6e−81 | |
| 2956 | 3.622369807 | up | 0.0000125 | 0.001058667 | BE821955 | 647 | 135 | 142 | 95 | 2e−73 | protein biosynthesis |
| 2957 | 3.625992673 | up | 0.0000202 | 0.001260859 | BM094345 | 545 | 22 | 32 | 68 | 1e−05 | |
| 2958 | 3.626559843 | up | 0.003106681 | 0.021476697 | CD391589 | 635 | 63 | 124 | 50 | 1e−32 | |
| 2959 | 3.630204158 | up | 0.006555304 | 0.035519132 | CD395146 | 679 | 70 | 85 | 82 | 5e−36 | |
| 2960 | 3.632512564 | up | 0.000000439 | 0.00067462 | BE823381 | 632 | 58 | 147 | 39 | 6e−30 | regulation of transcription\, DNA- |
| | | | | | | | | | | | dependent |
| 2961 | 3.635505657 | up | 0.000196447 | 0.003801663 | BM893332 | 560 | 12 | 25 | 48 | 1.1 | |
| 2962 | 3.63623653 | up | 0.0000878 | 0.002491894 | AW310058 | 692 | 29 | 59 | 49 | 9e−12 | chloroplast transport |
| 2963 | 3.63738586 | up | 0.0000249 | 0.001364741 | AW351098 | 689 | 90 | 101 | 89 | 8e−48 | protein biosynthesis |
| 2964 | 3.638901877 | up | 0.0000432 | 0.001770252 | BE820335 | 519 | 63 | 92 | 68 | 2e−31 | |
| 2965 | 3.640977315 | up | 0.0000192 | 0.001231955 | AW472039 | 449 | 65 | 84 | 77 | 3e−33 | protein biosynthesis |
| 2966 | 3.642425494 | up | 0.00000852 | 0.000900892 | AI437954 | 436 | 20 | 59 | 33 | 0.009 | Serine O-acetyltransferase 1 (SAT1), |
| | | | | | | | | | | | cellular response to sulfate starvation |
| 2967 | 3.645776847 | up | 0.0000678 | 0.002182183 | BU549208 | 647 | 16 | 25 | 64 | 1.4 | |
| 2968 | 3.649887246 | up | 0.00000852 | 0.000900892 | CD395394 | 386 | 10 | 27 | 37 | 1.9 | DNA recombination |
| 2969 | 3.650069203 | up | 0.000000275 | 0.00067462 | BI968721 | 802 | 118 | 195 | 60 | 2e−57 | |
| 2970 | 3.650998616 | up | 0.0000325 | 0.001543897 | BE824051 | 487 | 67 | 88 | 76 | 2e−35 | protein biosynthesis |
| 2971 | 3.653650366 | up | 0.000149131 | 0.003253718 | CD390917 | 668 | 145 | 164 | 88 | 4e−81 | protein biosynthesis |
| 2972 | 3.658979605 | up | 0.000105402 | 0.002709915 | CF808827 | 577 | 131 | 134 | 97 | 5e−73 | |
| 2973 | 3.659680032 | up | 0.000132466 | 0.003049321 | CA936708 | 414 | 15 | 43 | 34 | 2.9 | |
| 2974 | 3.663999129 | up | 0.000045 | 0.001793484 | CD408467 | 543 | 47 | 67 | 70 | 1e−22 | |
| 2975 | 3.667651372 | up | 0.0000249 | 0.001364741 | BM523799 | 401 | 44 | 50 | 88 | 2e−20 | protein biosynthesis |
| 2976 | 3.667795056 | up | 0.000021 | 0.001283285 | BE330720 | 494 | 142 | 164 | 86 | 2e−80 | lysine biosynthesis via |
| | | | | | | | | | | | diaminopimelate |
| 2977 | 3.672197629 | up | 0.0000786 | 0.002362551 | BU551341 | 544 | 12 | 19 | 63 | 0.16 | |
| 2978 | 3.674277201 | up | 0.000142828 | 0.003175131 | CD405543 | 635 | 71 | 99 | 71 | 8e−39 | translational elongation |
| 2979 | 3.676757637 | up | 0.00000613 | 0.000832282 | BU549263 | 625 | 75 | 87 | 86 | 6e−39 | protein biosynthesis |
| 2980 | 3.679041804 | up | 0.0000134 | 0.001091338 | BI968058 | 741 | 56 | 110 | 50 | 1e−24 | fatty acid biosynthesis |
| 2981 | 3.680173728 | up | 0.0000678 | 0.002182183 | CD391374 | 674 | 129 | 152 | 84 | 5e−71 | protein biosynthesis |
| 2982 | 3.680908065 | up | 0.00000378 | 0.000764834 | CD405557 | 707 | 124 | 134 | 92 | 1e−68 | |
| 2983 | 3.683142909 | up | 0.0000486 | 0.001864758 | CD396345 | 645 | 108 | 126 | 85 | 6e−63 | |
| 2984 | 3.684895173 | up | 0.000049 | 0.001870412 | AW309620 | 459 | 18 | 34 | 52 | 7e−04 | |
| 2985 | 3.685961635 | up | 0.0000226 | 0.001321932 | CD415189 | 656 | 91 | 104 | 87 | 4e−48 | protein biosynthesis |
| 2986 | 3.686437379 | up | 0.0000124 | 0.001058667 | CD401465 | 581 | 110 | 144 | 76 | 9e−62 | |
| 2987 | 3.688758772 | up | 0.000012 | 0.00104082 | BE190254 | 416 | 50 | 62 | 80 | 4e−23 | |
| 2988 | 3.688816184 | up | 0.0000289 | 0.001464287 | CA935467 | 432 | 31 | 51 | 60 | 7e−11 | protein biosynthesis |
| 2989 | 3.689663573 | up | 0.00000936 | 0.000924406 | CK605999 | 443 | 51 | 62 | 82 | 5e−23 | |
| 2990 | 3.696120979 | up | 0.00000702 | 0.000873658 | BE657697 | 515 | 70 | 85 | 82 | 6e−34 | RNA processing |
| 2991 | 3.696846938 | up | 0.000000684 | 0.000677501 | AI442819 | 683 | 81 | 143 | 56 | 3e−40 | DNA metabolism |
| 2992 | 3.698101312 | up | 0.000133676 | 0.003067769 | CF808199 | 756 | 230 | 230 | 100 | e−134 | microtubule-based process |
| 2993 | 3.698680592 | up | 0.0000582 | 0.00202784 | CF807433 | 652 | 129 | 139 | 92 | 7e−72 | |
| 2994 | 3.701762616 | up | 0.0000053 | 0.000810852 | BU545654 | 588 | 36 | 52 | 69 | 8e−18 | |
| 2995 | 3.703124096 | up | 0.000114557 | 0.002833761 | CD417784 | 423 | 52 | 56 | 92 | 2e−28 | protein biosynthesis |
| 2996 | 3.704956388 | up | 0.00000033 | 0.00067462 | CD403248 | 469 | 15 | 48 | 31 | 0.78 | proteolysis and peptidolysis |
| 2997 | 3.70543769 | up | 0.000100605 | 0.002653677 | BG651436 | 191 | 8 | 20 | 40 | 7.1 | |
| 2998 | 3.705520722 | up | 0.0000448 | 0.00179078 | CD410971 | 442 | 24 | 42 | 57 | 1e−07 | |
| 2999 | 3.708944546 | up | 0.0000492 | 0.001873618 | AW348645 | 590 | 28 | 51 | 54 | 2e−06 | |
| 3000 | 3.711274742 | up | 0.0000152 | 0.00113073 | BG507769 | 279 | 19 | 62 | 30 | 2.4 | |
| 3001 | 3.71170005 | up | 0.00000272 | 0.000720842 | BE658301 | 418 | 25 | 80 | 31 | 0.006 | regulation of transcription\, DNA- |
| | | | | | | | | | | | dependent |
| 3002 | 3.711933864 | up | 0.00000681 | 0.000870238 | CF807792 | 604 | 102 | 136 | 75 | 9e−55 | Syringolide-induced protein 14-1-1, |
| | | | | | | | | | | | protein biosynthesis |
| 3003 | 3.715206359 | up | 0.000200698 | 0.0038555 | BF596776 | 473 | 130 | 157 | 82 | 4e−76 | |
| 3004 | 3.717581914 | up | 0.0000133 | 0.001087368 | CF806905 | 380 | 46 | 51 | 90 | 2e−23 | |
| 3005 | 3.717972842 | up | 0.00000216 | 0.000707618 | BM519648 | 587 | 126 | 187 | 67 | 6e−69 | chloroplast transport |
| 3006 | 3.719556858 | up | 0.00000493 | 0.000799682 | CD399094 | 595 | 96 | 105 | 91 | 1e−54 | protein biosynthesis |
| 3007 | 3.726346785 | up | 0.0000451 | 0.001793484 | BG882474 | 426 | 12 | 35 | 34 | 1.4 | |
| 3008 | 3.726595019 | up | 0.0000406 | 0.00172837 | CF806121 | 683 | 150 | 201 | 74 | 2e−76 | ATP/ADP exchange |
| 3009 | 3.726933553 | up | 0.00000998 | 0.000951145 | CD402535 | 721 | 141 | 167 | 84 | 3e−77 | |
| 3010 | 3.727036741 | up | 0.000327004 | 0.005155046 | CF806892 | 678 | 134 | 224 | 59 | 1e−71 | proteolysis and peptidolysis |
| 3011 | 3.728655348 | up | 0.00000559 | 0.000820095 | CA785635 | 506 | 66 | 115 | 57 | 1e−34 | |
| 3012 | 3.728914734 | up | 0.0000721 | 0.002257131 | CD406020 | 459 | 32 | 50 | 64 | 1e−12 | transport |
| 3013 | 3.729431984 | up | 0.0000778 | 0.002341624 | AW756936 | 583 | 115 | 165 | 69 | 1e−62 | electron transport |
| 3014 | 3.731545091 | up | 0.0000154 | 0.001135492 | BE823654 | 625 | 101 | 144 | 70 | 5e−53 | protein biosynthesis |
| 3015 | 3.736355498 | up | 0.00000876 | 0.00091304 | CD392718 | 711 | 128 | 149 | 85 | 2e−70 | protein biosynthesis |
| 3016 | 3.736921668 | up | 0.0000435 | 0.001771311 | CD392318 | 656 | 90 | 118 | 76 | 3e−50 | |
| 3017 | 3.741086389 | up | 0.00000283 | 0.000720842 | BG551013 | 403 | 27 | 34 | 79 | 6e−11 | |
| 3018 | 3.746436218 | up | 0.00000628 | 0.000834433 | AW348613 | 748 | 130 | 165 | 78 | 2e−75 | |
| 3019 | 3.747841398 | up | 0.00000284 | 0.000720842 | BI973833 | 576 | 80 | 116 | 68 | 1e−43 | |
| 3020 | 3.749676196 | up | 0.00000316 | 0.000720842 | BI943980 | 377 | 33 | 39 | 84 | 5e−10 | regulation of transcription\, DNA- |
| | | | | | | | | | | | dependent |
| 3021 | 3.750047638 | up | 0.00069713 | 0.008279717 | CD400030 | 317 | 12 | 31 | 38 | 1.4 | regulation of transcription |
| 3022 | 3.750269938 | up | 0.00000548 | 0.000814881 | BG154613 | 260 | 12 | 27 | 44 | 3.2 | |
| 3023 | 3.75171098 | up | 0.0000141 | 0.001112691 | AW568434 | 514 | 53 | 61 | 86 | 5e−26 | |
| 3024 | 3.752502303 | up | 0.000156724 | 0.003343945 | BE823140 | 641 | 100 | 144 | 69 | 7e−56 | carbohydrate metabolism |
| 3025 | 3.753273962 | up | 0.0000427 | 0.001761872 | BG237519 | 511 | 94 | 155 | 60 | 6e−55 | |
| 3026 | 3.755956861 | up | 0.001805402 | 0.015074698 | BU550576 | 607 | 87 | 106 | 82 | 1e−49 | |
| 3027 | 3.757551437 | up | 0.000000877 | 0.000704945 | BM731809 | 423 | 17 | 25 | 68 | 5e−04 | aspartate metabolism |
| 3028 | 3.758035345 | up | 0.0000184 | 0.00122348 | CB063421 | 575 | 83 | 117 | 70 | 2e−43 | protein biosynthesis |
| 3029 | 3.760337166 | up | 0.00000513 | 0.000800719 | BI787878 | 497 | 51 | 67 | 76 | 1e−25 | |
| 3030 | 3.761107611 | up | 0.0000157 | 0.001142626 | BE657247 | 835 | 71 | 79 | 89 | 9e−36 | transport |
| 3031 | 3.763433179 | up | 0.00000289 | 0.000720842 | BE824014 | 654 | 120 | 154 | 77 | 2e−57 | ossification |
| 3032 | 3.76561069 | up | 0.000786242 | 0.008934649 | AW733377 | 250 | 12 | 31 | 38 | 1.4 | regulation of transcription |
| 3033 | 3.769532536 | up | 0.00000129 | 0.000704945 | AW666282 | 571 | 145 | 189 | 76 | 2e−77 | |
| 3034 | 3.772480851 | up | 0.00000646 | 0.000848516 | AW349133 | 664 | 63 | 126 | 50 | 6e−24 | |
| 3035 | 3.773269923 | up | 0.000182079 | 0.003660995 | BF595545 | 462 | 118 | 153 | 77 | 2e−66 | response to Wounding |
| 3036 | 3.778577134 | up | 0.00000693 | 0.000870238 | AW706534 | 214 | 15 | 42 | 35 | 3.3 | |
| 3037 | 3.778734755 | up | 0.00000134 | 0.000704945 | CD391304 | 590 | 96 | 135 | 71 | 4e−52 | protein biosynthesis |
| 3038 | 3.783166017 | up | 0.0000399 | 0.001711701 | BI095213 | 403 | 47 | 92 | 51 | 6e−21 | transport |
| 3039 | 3.785951161 | up | 0.00000177 | 0.000704945 | AW119809 | 380 | 9 | 17 | 52 | 1.4 | transport |
| 3040 | 3.786979833 | up | 0.0000261 | 0.001380871 | BU544012 | 572 | 58 | 78 | 74 | 3e−29 | nucleosome assembly |
| 3041 | 3.788939404 | up | 0.0000148 | 0.001127466 | BU926887 | 274 | 15 | 42 | 35 | 0.29 | |
| 3042 | 3.79030618 | up | 0.0000215 | 0.001295711 | BQ611280 | 443 | 17 | 46 | 36 | 4.4 | |
| 3043 | 3.794631549 | up | 0.00000407 | 0.000768376 | CD397451 | 575 | 41 | 56 | 73 | 9e−17 | protein biosynthesis |
| 3044 | 3.796193362 | up | 0.000796807 | 0.008980441 | CD398389 | 462 | 58 | 65 | 89 | 3e−28 | transcription initiation |
| 3045 | 3.797196083 | up | 0.0000982 | 0.002619922 | CD390917 | 668 | 145 | 164 | 88 | 4e−81 | protein biosynthesis |
| 3046 | 3.797539408 | up | 0.00000407 | 0.000768376 | CA785562 | 607 | 108 | 154 | 70 | 9e−60 | |
| 3047 | 3.800677593 | up | 0.00000832 | 0.000900892 | BE023586 | 385 | 14 | 40 | 35 | 3.1 | transport |
| 3048 | 3.8010012 | up | 0.0000369 | 0.001630669 | BE801051 | 468 | 110 | 157 | 70 | 9e−61 | response to heat |
| 3049 | 3.802321968 | up | 0.00000118 | 0.000704945 | BU084337 | 520 | 27 | 57 | 47 | 2e−06 | metabolism |
| 3050 | 3.808284761 | up | 0.0000685 | 0.002184143 | AW831739 | 588 | 92 | 109 | 84 | 1e−50 | chloroplast organization and |
| | | | | | | | | | | | biogenesis |
| 3051 | 3.808456636 | up | 0.0000119 | 0.001036088 | CD402535 | 721 | 141 | 167 | 84 | 3e−77 | |
| 3052 | 3.812633096 | up | 0.003637131 | 0.023804947 | BG653526 | 364 | 16 | 16 | 100 | 0.002 | response to oxidative stress |
| 3053 | 3.817043783 | up | 0.000053 | 0.001956938 | BE821880 | 706 | 74 | 118 | 62 | 1e−36 | |
| 3054 | 3.821875641 | up | 0.00000467 | 0.000797676 | AW459418 | 298 | 13 | 21 | 61 | 9.4 | regulation of transcription |
| 3055 | 3.822769381 | up | 0.000172973 | 0.003549485 | BM307710 | 192 | 14 | 40 | 35 | 4.2 | proteolysis and peptidolysis |
| 3056 | 3.829192364 | up | 0.00000093 | 0.000704945 | AW100894 | 361 | 16 | 44 | 36 | 2.0 | |
| 3057 | 3.837563051 | up | 0.0000549 | 0.001985296 | BE823590 | 604 | 109 | 137 | 79 | 5e−55 | protein biosynthesis |
| 3058 | 3.854086417 | up | 0.000000486 | 0.00067462 | AW317212 | 486 | 27 | 55 | 49 | 2e−10 | mitochondrial translocation |
| 3059 | 3.868068734 | up | 0.001406528 | 0.01280686 | AI856374 | 834 | 94 | 135 | 69 | 3e−51 | signal transduction |
| 3060 | 3.878130425 | up | 0.0000558 | 0.001994018 | BI945894 | 471 | 143 | 157 | 91 | 5e−77 | |
| 3061 | 3.879625869 | up | 0.00000391 | 0.000767688 | CK606431 | 532 | 59 | 86 | 68 | 1e−29 | protein biosynthesis |
| 3062 | 3.888415623 | up | 0.00000439 | 0.000778398 | BE824014 | 654 | 120 | 154 | 77 | 2e−57 | ossification |
| 3063 | 3.892763364 | up | 0.0000304 | 0.00148348 | AW349327 | 589 | 137 | 140 | 97 | 4e−76 | |
| 3064 | 3.893000206 | up | 0.000377952 | 0.005596014 | AI900736 | 497 | 14 | 42 | 33 | 0.40 | |
| 3065 | 3.905955798 | up | 0.00000417 | 0.000769894 | CD391107 | 594 | 41 | 66 | 62 | 2e−19 | protein biosynthesis |
| 3066 | 3.906848263 | up | 0.00000698 | 0.000872224 | BE823925 | 633 | 33 | 77 | 42 | 4e−09 | regulation of transcription |
| 3067 | 3.91521045 | up | 0.00000141 | 0.000704945 | AW186460 | 659 | 105 | 186 | 56 | 1e−47 | protein biosynthesis |
| 3068 | 3.919315634 | up | 0.0000529 | 0.001955984 | CK606099 | 810 | 142 | 172 | 82 | 3e−80 | protein biosynthesis |
| 3069 | 3.919494015 | up | 0.0000261 | 0.001380871 | BQ453310 | 558 | 51 | 55 | 92 | 4e−22 | protein biosynthesis |
| 3070 | 3.919980014 | up | 0.001446448 | 0.01300662 | BG725933 | 518 | 112 | 179 | 62 | 6e−61 | |
| 3071 | 3.920410544 | up | 0.000000798 | 0.000704945 | BQ252691 | 557 | 21 | 41 | 51 | 4e−06 | |
| 3072 | 3.922246355 | up | 0.0000682 | 0.002184143 | AW351315 | 652 | 111 | 129 | 86 | 2e−58 | protein biosynthesis |
| 3073 | 3.926486932 | up | 0.000670758 | 0.008048881 | BM093489 | 560 | 51 | 57 | 89 | 2e−24 | transcription initiation |
| 3074 | 3.926694025 | up | 0.0000461 | 0.001810721 | BM891517 | 428 | 16 | 20 | 80 | 5e−04 | nucleosome assembly |
| 3075 | 3.927646225 | up | 0.00000433 | 0.000776405 | BM525760 | 527 | 84 | 107 | 78 | 4e−43 | copper ion transport |
| 3076 | 3.927944991 | up | 0.0000152 | 0.00113073 | AW350307 | 551 | 40 | 64 | 62 | 2e−15 | |
| 3077 | 3.930932064 | up | 0.000421234 | 0.005990282 | CD396375 | 694 | 74 | 164 | 45 | 4e−36 | protein folding |
| 3078 | 3.933868614 | up | 0.00000334 | 0.000747115 | BU082303 | 569 | 21 | 26 | 80 | 1e−06 | |
| 3079 | 3.935244215 | up | 0.0000202 | 0.001260859 | CK606509 | 380 | 52 | 66 | 78 | 8e−25 | protein biosynthesis |
| 3080 | 3.936516929 | up | 0.00000754 | 0.000889839 | AW348576 | 650 | 22 | 33 | 66 | 6e−05 | transport |
| 3081 | 3.939341312 | up | 0.00058386 | 0.007382488 | BE824203 | 842 | 167 | 269 | 62 | 2e−95 | |
| 3082 | 3.940267112 | up | 0.000475849 | 0.00648603 | BQ253682 | 413 | 75 | 103 | 72 | 1e−40 | |
| 3083 | 3.940557483 | up | 0.002904648 | 0.020529018 | BI470908 | 313 | 20 | 56 | 35 | 0.49 | carbohydrate metabolism |
| 3084 | 3.946451935 | up | 0.00000185 | 0.000704945 | BM270192 | 317 | 13 | 29 | 44 | 2.4 | dicarboxylic acid transport |
| 3085 | 3.954446199 | up | 0.0000175 | 0.001205969 | AW348183 | 676 | 98 | 144 | 68 | 2e−51 | protein biosynthesis |
| 3086 | 3.955852855 | up | 0.0000203 | 0.00126135 | CF808247 | 551 | 132 | 159 | 83 | 1e−73 | Ribosomal protein S11 mRNA, 3′ |
| | | | | | | | | | | | end. |
| 3087 | 3.956039153 | up | 0.00000125 | 0.000704945 | BI317633 | 415 | 15 | 47 | 31 | 1.3 | |
| 3088 | 3.957618001 | up | 0.0000233 | 0.001330585 | AW307023 | 360 | 12 | 20 | 60 | 4.4 | |
| 3089 | 3.960329622 | up | 0.0000658 | 0.002159139 | BI945653 | 348 | 9 | 16 | 56 | 4.2 | Mo-molybdopterin cofactor |
| | | | | | | | | | | | biosynthesis |
| 3090 | 3.96062563 | up | 0.000033 | 0.001543897 | BI967272 | 744 | 81 | 96 | 84 | 8e−43 | |
| 3091 | 3.961282127 | up | 0.001087279 | 0.010938585 | BG405072 | 487 | 34 | 53 | 64 | 1e−14 | |
| 3092 | 3.962647892 | up | 0.00000305 | 0.000720842 | AW317399 | 677 | 35 | 61 | 57 | 1e−14 | |
| 3093 | 3.965843652 | up | 0.000263379 | 0.004527755 | BE807671 | 563 | 136 | 193 | 70 | 2e−74 | Carbohydrate metabolism |
| 3094 | 3.968876964 | up | 0.00000234 | 0.000707618 | CA937278 | 556 | 102 | 185 | 55 | 6e−52 | regulation of transcription |
| 3095 | 3.968966676 | up | 0.00000985 | 0.000947882 | BQ080704 | 342 | 11 | 18 | 61 | 7.2 | cation transport |
| 3096 | 3.972586943 | up | 0.00000173 | 0.000704945 | CD392596 | 450 | 87 | 95 | 91 | 1e−42 | protein biosynthesis |
| 3097 | 3.972999037 | up | 0.000169188 | 0.003500454 | BQ453224 | 402 | 15 | 31 | 48 | 6.0 | |
| 3098 | 3.975349936 | up | 0.0000649 | 0.002146014 | AW831739 | 588 | 92 | 109 | 84 | 1e−50 | chloroplast organization and |
| | | | | | | | | | | | biogenesis |
| 3099 | 3.977142401 | up | 0.00000535 | 0.000810852 | CD394907 | 582 | 58 | 94 | 61 | 6e−26 | mitochondrial translocation |
| 3100 | 3.979880807 | up | 0.0000189 | 0.001231955 | CD390734 | 607 | 80 | 91 | 87 | 4e−44 | protein biosynthesis |
| 3101 | 3.982511546 | up | 0.0000406 | 0.001729476 | BI972560 | 448 | 115 | 149 | 77 | 2e−61 | RNA processing |
| 3102 | 3.986261312 | up | 0.000000753 | 0.000704945 | CD409963 | 626 | 75 | 129 | 58 | 1e−38 | |
| 3103 | 3.991321578 | up | 0.0000749 | 0.002299445 | CD395859 | 700 | 68 | 94 | 72 | 8e−37 | |
| 3104 | 3.993624657 | up | 0.00000861 | 0.000900892 | CF921581 | 573 | 52 | 64 | 81 | 2e−25 | |
| 3105 | 4.000920943 | up | 0.00000397 | 0.000767688 | BE822592 | 673 | 13 | 40 | 32 | 2.0 | protein amino acid glycosylation |
| 3106 | 4.007050993 | up | 0.0000158 | 0.001142626 | AW309369 | 651 | 123 | 146 | 84 | 6e−70 | protein biosynthesis |
| 3107 | 4.009024107 | up | 0.0000334 | 0.001546189 | BE020799 | 479 | 98 | 154 | 63 | 8e−57 | |
| 3108 | 4.011408828 | up | 0.0000149 | 0.001128257 | CD405473 | 665 | 131 | 163 | 80 | 9e−72 | protein biosynthesis |
| 3109 | 4.013945921 | up | 0.0000245 | 0.001353718 | CD393418 | 644 | 110 | 124 | 88 | 3e−56 | protein biosynthesis |
| 3110 | 4.014074782 | up | 0.00000144 | 0.000704945 | CA937458 | 553 | 43 | 70 | 61 | 3e−18 | protein biosynthesis |
| 3111 | 4.016177783 | up | 0.00000224 | 0.000707618 | AW309535 | 599 | 96 | 109 | 88 | 6e−53 | |
| 3112 | 4.018929012 | up | 0.0000155 | 0.001135545 | CF806800 | 714 | 160 | 189 | 84 | 2e−86 | |
| 3113 | 4.024490791 | up | 0.0000168 | 0.001179784 | BI967990 | 769 | 15 | 51 | 29 | 2.4 | protein biosynthesis |
| 3114 | 4.035718215 | up | 0.0000332 | 0.001543897 | BE800329 | 403 | 17 | 62 | 27 | 2.1 | |
| 3115 | 4.039763352 | up | 0.00000254 | 0.000712455 | BU578317 | 508 | 91 | 149 | 61 | 2e−52 | |
| 3116 | 4.04294671 | up | 0.003485888 | 0.023101992 | BG363116 | 562 | 39 | 74 | 52 | 2e−15 | carbohydrate metabolism |
| 3117 | 4.043292064 | up | 0.00000452 | 0.000784876 | BG508333 | 391 | 13 | 32 | 40 | 5.6 | cell death |
| 3118 | 4.050865079 | up | 0.0000447 | 0.001789988 | CD410787 | 367 | 10 | 39 | 25 | 6.1 | regulation of transcription\, DNA- |
| | | | | | | | | | | | dependent |
| 3119 | 4.056663563 | up | 0.001967893 | 0.015938938 | BG405072 | 487 | 34 | 53 | 64 | 1e−14 | |
| 3120 | 4.065834946 | up | 0.00000413 | 0.000769273 | BM094850 | 530 | 70 | 152 | 46 | 2e−27 | |
| 3121 | 4.067472918 | up | 0.00154366 | 0.013582038 | BG653508 | 394 | 18 | 29 | 62 | 0.002 | |
| 3122 | 4.06757738 | up | 0.00000235 | 0.000707618 | BU084833 | 572 | 51 | 78 | 65 | 4e−25 | transport |
| 3123 | 4.068901166 | up | 0.000032 | 0.001530557 | BG363176 | 504 | 13 | 39 | 33 | 2.0 | cellulose biosynthesis |
| 3124 | 4.076430939 | up | 0.0000416 | 0.001748981 | CF807374 | 713 | 210 | 223 | 94 | e−127 | ribosomal protein L2 |
| 3125 | 4.077602214 | up | 0.000668402 | 0.008040067 | AW759068 | 562 | 40 | 51 | 78 | 8e−18 | protein folding |
| 3126 | 4.078395606 | up | 0.0000207 | 0.001280424 | BU549461 | 597 | 86 | 146 | 58 | 6e−51 | electron transport |
| 3127 | 4.083612848 | up | 0.0000223 | 0.001314393 | BG725712 | 433 | 13 | 36 | 36 | 0.17 | protein amino acid phosphorylation |
| 3128 | 4.088223991 | up | 0.0000298 | 0.00147316 | BE609165 | 632 | 123 | 173 | 71 | 6e−71 | translational elongation |
| 3129 | 4.088447576 | up | 0.0000289 | 0.001464291 | CF808081 | 538 | 114 | 153 | 74 | 2e−65 | carbohydrate metabolism |
| 3130 | 4.089228902 | up | 0.00000642 | 0.000848516 | BG507699 | 405 | 85 | 117 | 72 | 2e−47 | carbohydrate metabolism |
| 3131 | 4.093904653 | up | 0.003265008 | 0.022199931 | CA800128 | 324 | 9 | 13 | 69 | 1.4 | regulation of transcription\, DNA- |
| | | | | | | | | | | | dependent |
| 3132 | 4.095224735 | up | 0.000000911 | 0.000704945 | BM307355 | | | | | | |
| 3133 | 4.096660291 | up | 0.000131577 | 0.003040011 | BE612060 | 321 | 23 | 57 | 40 | 5e−04 | |
| 3134 | 4.099447411 | up | 0.00002 | 0.001256547 | BU964883 | 454 | 97 | 109 | 88 | 4e−53 | RNA processing |
| 3135 | 4.105271727 | up | 0.001143892 | 0.011304876 | BE807258 | 317 | 8 | 14 | 57 | 4.2 | |
| 3136 | 4.108482392 | up | 0.000328704 | 0.005168841 | BI970516 | 792 | 115 | 168 | 68 | 3e−57 | |
| 3137 | 4.111999481 | up | 0.0000355 | 0.001589423 | CF807971 | 241 | 34 | 45 | 75 | 1e−13 | protein biosynthesis |
| 3138 | 4.133442717 | up | 0.00000366 | 0.000764834 | CD402535 | 721 | 141 | 167 | 84 | 3e−77 | |
| 3139 | 4.1336298 | up | 0.0000146 | 0.001125207 | BG507968 | 479 | 17 | 48 | 35 | 2.4 | |
| 3140 | 4.135106783 | up | 0.0000424 | 0.001755385 | AI442912 | 610 | 85 | 103 | 82 | 2e−44 | protein biosynthesis |
| 3141 | 4.136800438 | up | 0.0000544 | 0.001979942 | BI786435 | 533 | 17 | 34 | 50 | 0.092 | glycolysis |
| 3142 | 4.140172143 | up | 0.000085 | 0.002462638 | CD395444 | 605 | 46 | 111 | 41 | 3e−14 | protein biosynthesis |
| 3143 | 4.143787569 | up | 0.0000525 | 0.001952406 | BQ296762 | 576 | 156 | 191 | 81 | 8e−82 | protein folding |
| 3144 | 4.147307188 | up | 0.000343456 | 0.005294571 | CD409965 | 600 | 89 | 138 | 64 | 2e−44 | |
| 3145 | 4.148437181 | up | 0.00000354 | 0.000764744 | BI941951 | 557 | 127 | 141 | 90 | 1e−70 | |
| 3146 | 4.151224211 | up | 0.0000135 | 0.001094185 | BI971017 | 791 | 121 | 201 | 60 | 4e−62 | metabolism |
| 3147 | 4.159165181 | up | 0.000110578 | 0.002790944 | CF807216 | 125 | 35 | 41 | 85 | 6e−15 | ATP/ADP exchange |
| 3148 | 4.16484666 | up | 0.00179323 | 0.015012473 | AY204711.1 | | | | | | |
| 3149 | 4.164916852 | up | 0.00000369 | 0.000764834 | BE657319 | 753 | 130 | 177 | 73 | 1e−67 | |
| 3150 | 4.166823048 | up | 0.00000274 | 0.000720842 | BU547182 | 481 | 11 | 33 | 33 | 1.8 | |
| 3151 | 4.167698751 | up | 0.0000141 | 0.001112691 | BQ627738 | 317 | 13 | 25 | 52 | 3.2 | protein amino acid phosphorylation |
| 3152 | 4.17272944 | up | 0.000000269 | 0.00067462 | BQ611057 | 445 | 120 | 141 | 85 | 7e−65 | selenocysteine incorporation |
| 3153 | 4.174995957 | up | 0.0000223 | 0.001314393 | CF807294 | 710 | 191 | 204 | 93 | e−109 | |
| 3154 | 4.183634147 | up | 0.000111442 | 0.002796365 | BI943748 | 386 | 97 | 128 | 75 | 3e−50 | |
| 3155 | 4.187002697 | up | 0.00000234 | 0.000707618 | BI893680 | 472 | 108 | 144 | 75 | 9e−66 | |
| 3156 | 4.191833347 | up | 0.00000337 | 0.000749527 | CA852593 | 561 | 16 | 65 | 24 | 0.13 | |
| 3157 | 4.197402141 | up | 0.00000773 | 0.000894724 | BG650384 | 417 | 17 | 45 | 37 | 0.21 | |
| 3158 | 4.211303138 | up | 0.0000107 | 0.000974923 | CD406719 | 632 | 89 | 95 | 93 | 3e−47 | protein biosynthesis |
| 3159 | 4.213573595 | up | 0.00000502 | 0.000799682 | BU084337 | 520 | 27 | 57 | 47 | 2e−06 | metabolism |
| 3160 | 4.216316952 | up | 0.000018 | 0.00121727 | CD395501 | 746 | 123 | 143 | 86 | 6e−71 | protein biosynthesis |
| 3161 | 4.222820301 | up | 0.00000382 | 0.000764834 | CD403850 | 556 | 58 | 92 | 63 | 2e−25 | |
| 3162 | 4.243318707 | up | 0.00000245 | 0.000712455 | BM086300 | 358 | 27 | 43 | 62 | 4e−11 | |
| 3163 | 4.25109603 | up | 0.0000128 | 0.001070251 | AW704299 | 503 | 94 | 143 | 65 | 4e−45 | |
| 3164 | 4.256737771 | up | 0.00000376 | 0.000764834 | CD404419 | 523 | 27 | 36 | 75 | 4e−09 | regulation of transcription\, DNA- |
| | | | | | | | | | | | dependent |
| 3165 | 4.259555969 | up | 0.004386989 | 0.027082256 | BE820833 | 537 | 49 | 81 | 60 | 5e−22 | |
| 3166 | 4.260143949 | up | 0.00000256 | 0.000712455 | BE659805 | 586 | 61 | 75 | 81 | 2e−31 | |
| 3167 | 4.265987289 | up | 0.0000067 | 0.000860462 | BM568081 | 278 | 15 | 47 | 31 | 2.4 | response to pathogenic fungi |
| 3168 | 4.272131853 | up | 0.0000181 | 0.001219768 | CF806669 | 666 | 151 | 180 | 83 | 5e−87 | |
| 3169 | 4.276548836 | up | 0.0000215 | 0.001295711 | AW119405 | 462 | 98 | 120 | 81 | 6e−56 | sulfate assimilation |
| 3170 | 4.28010433 | up | 0.0000226 | 0.001321932 | CF806123 | 285 | 90 | 92 | 97 | 1e−49 | |
| 3171 | 4.289144714 | up | 0.0000659 | 0.002161506 | BU090070 | 553 | 35 | 63 | 55 | 6e−12 | protein biosynthesis |
| 3172 | 4.291945436 | up | 0.002791178 | 0.020034435 | AW101841 | 229 | 15 | 51 | 29 | 0.65 | |
| 3173 | 4.292131916 | up | 0.000168103 | 0.00348952 | CF807412 | 567 | 54 | 126 | 42 | 1e−22 | electron transport |
| 3174 | 4.296855044 | up | 0.00000384 | 0.000764834 | CA937458 | 553 | 43 | 70 | 61 | 3e−18 | protein biosynthesis |
| 3175 | 4.301917549 | up | 0.0000132 | 0.001087368 | BI469904 | 537 | 156 | 179 | 87 | 4e−84 | |
| 3176 | 4.302981135 | up | 0.0000191 | 0.001231955 | BI471150 | 564 | 155 | 188 | 82 | 3e−87 | |
| 3177 | 4.30449067 | up | 0.0000103 | 0.000963549 | AW348863 | 719 | 152 | 191 | 79 | 8e−86 | 40S ribosomal S4 protein |
| 3178 | 4.306710845 | up | 0.000094 | 0.002557182 | AF135862.1 | | | | | | precursor monofunctional |
| | | | | | | | | | | | aspartokinase |
| 3179 | 4.309432882 | up | 0.000144195 | 0.003182607 | AW396703 | 338 | 12 | 44 | 27 | 5.5 | |
| 3180 | 4.313755339 | up | 0.000204494 | 0.003904488 | CD394756 | 679 | 115 | 119 | 96 | 7e−64 | response to high light intensity |
| 3181 | 4.317419398 | up | 0.00000644 | 0.000848516 | BE059545 | 425 | 36 | 62 | 58 | 2e−12 | |
| 3182 | 4.325677354 | up | 0.000646144 | 0.007896056 | CD413164 | 678 | 45 | 102 | 44 | 2e−14 | electron transport |
| 3183 | 4.336963481 | up | 0.0000355 | 0.001589423 | CD399436 | 631 | 64 | 69 | 92 | 9e−32 | protein biosynthesis |
| 3184 | 4.33738982 | up | 0.0000131 | 0.001083311 | CD417934 | 520 | 63 | 91 | 69 | 5e−31 | protein biosynthesis |
| 3185 | 4.338190068 | up | 0.0000342 | 0.00156033 | AW350453 | 679 | 122 | 141 | 86 | 8e−69 | protein biosynthesis |
| 3186 | 4.339633193 | up | 0.0000233 | 0.001330585 | CA934965 | 359 | 11 | 30 | 36 | 0.68 | |
| 3187 | 4.343859444 | up | 0.00000941 | 0.000924632 | BM092170 | 301 | 19 | 63 | 30 | 2.5 | |
| 3188 | 4.346114963 | up | 0.000160078 | 0.00338004 | AW395106 | 324 | 16 | 66 | 24 | 2.4 | |
| 3189 | 4.347934168 | up | 0.001858598 | 0.015317551 | AI938073 | 556 | 85 | 123 | 69 | 3e−42 | |
| 3190 | 4.356299309 | up | 0.00000814 | 0.000899886 | BU547551 | 589 | 120 | 148 | 81 | 1e−64 | protein biosynthesis |
| 3191 | 4.359318575 | up | 0.0000157 | 0.001139263 | CD415864 | 585 | 82 | 114 | 71 | 1e−40 | protein biosynthesis |
| 3192 | 4.363115461 | up | 0.00000828 | 0.000900892 | AI437596 | 522 | 99 | 138 | 71 | 2e−53 | |
| 3193 | 4.363836066 | up | 0.0000362 | 0.001613273 | CF808079 | 551 | 167 | 183 | 91 | 1e−90 | acidic ribosomal protein P0, protein |
| | | | | | | | | | | | biosynthesis |
| 3194 | 4.364887513 | up | 0.00000928 | 0.000924406 | AW310192 | | | | | | |
| 3195 | 4.365855949 | up | 0.0000011 | 0.000704945 | AF532618.1 | | | | | | nucleolar histone deacetylase HD2- |
| | | | | | | | | | | | P39 |
| 3196 | 4.375058368 | up | 0.0000463 | 0.001810721 | CF807971 | 241 | 34 | 45 | 75 | 1e−13 | protein biosynthesis |
| 3197 | 4.379646707 | up | 0.00000187 | 0.000704945 | BI470690 | 564 | 60 | 82 | 73 | 1e−32 | |
| 3198 | 4.401082274 | up | 0.001202941 | 0.011637232 | BG652966 | 458 | 78 | 125 | 62 | 2e−45 | transport |
| 3199 | 4.40722113 | up | 0.0000183 | 0.00122288 | BE807239 | 346 | 11 | 34 | 32 | 2.5 | response to oxidative stress |
| 3200 | 4.407283187 | up | 0.00000447 | 0.000784842 | AW734088 | 585 | 93 | 184 | 50 | 4e−42 | proteolysis and peptidolysis |
| 3201 | 4.409574616 | up | 0.0000151 | 0.001128338 | CF807857 | 614 | 162 | 183 | 88 | 5e−94 | |
| 3202 | 4.410992013 | up | 0.0000771 | 0.002341624 | BF069083 | 434 | 40 | 52 | 76 | 5e−20 | proteolysis and peptidolysis |
| 3203 | 4.414647238 | up | 0.00000043 | 0.00067462 | BU081368 | 563 | 124 | 175 | 70 | 4e−64 | transcription |
| 3204 | 4.416346811 | up | 0.000689238 | 0.008217113 | AF243183.1 | | | | | | beta-ketoacyl-ACP synthetase I-2 |
| 3205 | 4.419132319 | up | 0.00000374 | 0.000764834 | BE823287 | 453 | 71 | 86 | 82 | 2e−37 | protein biosynthesis |
| 3206 | 4.424839464 | up | 0.0000079 | 0.000899116 | CD402878 | 620 | 136 | 139 | 97 | 1e−75 | |
| 3207 | 4.425591857 | up | 0.000760598 | 0.00876176 | AW596148 | 509 | 9 | 40 | 22 | 4.6 | |
| 3208 | 4.434151329 | up | 0.0000132 | 0.001087368 | AI965477 | 373 | 14 | 45 | 31 | 1.3 | protein amino acid phosphorylation |
| 3209 | 4.438761618 | up | 0.00000309 | 0.000720842 | CD402378 | 614 | 124 | 137 | 90 | 1e−71 | |
| 3210 | 4.440436608 | up | 0.0000145 | 0.001125207 | CD415864 | 585 | 82 | 114 | 71 | 1e−40 | protein biosynthesis |
| 3211 | 4.444989511 | up | 0.00000622 | 0.000832974 | CD396190 | 617 | 46 | 66 | 69 | 3e−23 | |
| 3212 | 4.466861824 | up | 0.00000731 | 0.000883808 | BU547551 | 589 | 120 | 148 | 81 | 1e−64 | protein biosynthesis |
| 3213 | 4.473307814 | up | 0.00000721 | 0.000883683 | CF807237 | 668 | 151 | 174 | 86 | 2e−84 | protein biosynthesis |
| 3214 | 4.480861658 | up | 0.0000138 | 0.001103751 | CD411438 | 608 | 106 | 115 | 92 | 1e−56 | protein biosynthesis |
| 3215 | 4.481257021 | up | 0.0000148 | 0.001127466 | AW507568 | 444 | 103 | 126 | 81 | 9e−57 | |
| 3216 | 4.482072208 | up | 0.0000172 | 0.00119252 | AI965923 | 470 | 30 | 61 | 49 | 1e−07 | protein biosynthesis |
| 3217 | 4.489292855 | up | 0.0000586 | 0.002032757 | AW309868 | 559 | 14 | 35 | 40 | 0.84 | protein amino acid phosphorylation |
| 3218 | 4.491157971 | up | 0.00000224 | 0.000707618 | CF808515 | 549 | 103 | 134 | 76 | 5e−56 | |
| 3219 | 4.494193083 | up | 0.0000489 | 0.001870412 | CF806907 | 696 | 193 | 232 | 83 | e−113 | |
| 3220 | 4.50179096 | up | 0.000000397 | 0.00067462 | BF423913 | 319 | 13 | 30 | 43 | 4.2 | |
| 3221 | 4.522128131 | up | 0.00004 | 0.001711701 | CF808422 | 150 | 33 | 37 | 89 | 9e−14 | |
| 3222 | 4.527690312 | up | 0.0000578 | 0.002027667 | BG237894 | 254 | 10 | 20 | 50 | 3.3 | |
| 3223 | 4.53551868 | up | 0.00000027 | 0.00067462 | CD406619 | 616 | 78 | 99 | 78 | 7e−44 | metabolism |
| 3224 | 4.54073552 | up | 0.001069911 | 0.010859292 | CD397377 | 457 | 10 | 21 | 47 | 4.7 | |
| 3225 | 4.544529801 | up | 0.00000306 | 0.000720842 | AW317399 | 677 | 35 | 61 | 57 | 1e−14 | |
| 3226 | 4.553674927 | up | 0.00000832 | 0.000900892 | CK605966 | 547 | 52 | 68 | 76 | 7e−24 | translational elongation |
| 3227 | 4.575345118 | up | 0.0000868 | 0.002476129 | CK605922 | 645 | 108 | 123 | 87 | 2e−54 | protein biosynthesis |
| 3228 | 4.575521811 | up | 0.00000502 | 0.000799682 | BQ612571 | 578 | 97 | 167 | 58 | 2e−58 | |
| 3229 | 4.581539244 | up | 0.000165848 | 0.003459907 | BM887020 | 541 | 125 | 183 | 68 | 3e−78 | |
| 3230 | 4.600277279 | up | 0.00014256 | 0.003172939 | BU546744 | 627 | 117 | 136 | 86 | 2e−58 | chloroplast organization and |
| | | | | | | | | | | | biogenesis |
| 3231 | 4.609655727 | up | 0.0000112 | 0.000996489 | BE190185 | 606 | 108 | 198 | 54 | 3e−64 | |
| 3232 | 4.612184002 | up | 0.0000223 | 0.001314393 | BU545291 | 507 | 74 | 83 | 89 | 1e−38 | |
| 3233 | 4.619200497 | up | 0.0000759 | 0.00231972 | BI321851 | 427 | 11 | 24 | 45 | 9.0 | |
| 3234 | 4.63239391 | up | 0.0000251 | 0.001368772 | CD401290 | 657 | 86 | 122 | 70 | 3e−45 | protein biosynthesis |
| 3235 | 4.646848687 | up | 0.00247375 | 0.018515723 | AW101841 | 229 | 15 | 51 | 29 | 0.65 | |
| 3236 | 4.653441869 | up | 0.0000184 | 0.00122348 | BF325475 | 416 | 40 | 67 | 59 | 8e−19 | metabolism |
| 3237 | 4.666926038 | up | 0.0000681 | 0.002184143 | CF806473 | 516 | 113 | 122 | 92 | 6e−62 | protein biosynthesis |
| 3238 | 4.668047919 | up | 0.0000013 | 0.000704945 | AW309327 | 649 | 81 | 96 | 84 | 1e−42 | electron transport |
| 3239 | 4.675172297 | up | 0.001230554 | 0.01178662 | CD413725 | 674 | 94 | 137 | 68 | 9e−49 | |
| 3240 | 4.678335335 | up | 0.000601729 | 0.007558567 | AW569700 | 342 | 11 | 21 | 52 | 7.2 | carbohydrate metabolism |
| 3241 | 4.678525001 | up | 0.0000439 | 0.001772209 | BF423878 | 385 | 20 | 67 | 29 | 1.8 | regulation of transcription |
| 3242 | 4.684123382 | up | 0.00000799 | 0.000899116 | CD402690 | 569 | 55 | 77 | 71 | 2e−33 | |
| 3243 | 4.702208543 | up | 0.003918562 | 0.024977596 | BE823282 | 622 | 92 | 138 | 66 | 3e−47 | dicarboxylic acid transport |
| 3244 | 4.705938393 | up | 0.000298166 | 0.004858534 | BU579058 | 599 | 88 | 170 | 51 | 1e−42 | |
| 3245 | 4.713721563 | up | 0.000932219 | 0.009908017 | AW706352 | 257 | 7 | 14 | 50 | 7.2 | electron transport |
| 3246 | 4.719611733 | up | 0.00000316 | 0.000720842 | CD395421 | 737 | 110 | 163 | 67 | 2e−62 | |
| 3247 | 4.72522613 | up | 0.0000067 | 0.000860462 | CF805652 | 562 | 142 | 151 | 94 | 4e−83 | 40S ribosomal S4 protein |
| 3248 | 4.725468197 | up | 0.0000495 | 0.001881515 | BM524726 | 491 | 54 | 77 | 70 | 2e−25 | RNA processing |
| 3249 | 4.730566719 | up | 0.00000406 | 0.000768376 | CK605578 | 562 | 140 | 167 | 83 | 3e−77 | |
| 3250 | 4.730643647 | up | 0.0000632 | 0.002118327 | AW704088 | 598 | 94 | 110 | 85 | 3e−51 | protein biosynthesis |
| 3251 | 4.732959902 | up | 0.000089 | 0.002506437 | CF809354 | 546 | 109 | 167 | 65 | 7e−55 | |
| 3252 | 4.749982311 | up | 0.0000565 | 0.002008532 | BU926695 | 390 | 33 | 44 | 75 | 3e−15 | |
| 3253 | 4.764101006 | up | 0.00000287 | 0.000720842 | AW471533 | 545 | 127 | 165 | 76 | 4e−71 | nucleosome assembly |
| 3254 | 4.765209628 | up | 0.00000143 | 0.000704945 | BM521606 | 493 | 121 | 168 | 72 | 4e−64 | |
| 3255 | 4.768704706 | up | 0.0000013 | 0.000704945 | CD396190 | 617 | 46 | 66 | 69 | 3e−23 | |
| 3256 | 4.803343474 | up | 0.001421662 | 0.012876089 | L22162.1 | | | | | | brassinosteroid-regulated protein |
| 3257 | 4.843814036 | up | 0.000264706 | 0.004540648 | AY049920.1 | | | | | | ferritin |
| 3258 | 4.8495569 | up | 0.00000738 | 0.000884012 | CD393991 | 721 | 71 | 191 | 37 | 2e−22 | |
| 3259 | 4.859617204 | up | 0.0000372 | 0.001638946 | CF808471 | 537 | 89 | 104 | 85 | 8e−46 | |
| 3260 | 4.867131377 | up | 0.000155068 | 0.003324117 | AW394737 | 346 | 28 | 34 | 82 | 2e−11 | metabolism |
| 3261 | 4.874087385 | up | 0.0000138 | 0.001103751 | BG237280 | 231 | 14 | 35 | 40 | 7.1 | |
| 3262 | 4.891453181 | up | 0.00000305 | 0.000720842 | BG839298 | 563 | 16 | 55 | 29 | 7.2 | protein amino acid phosphorylation |
| 3263 | 4.911286989 | up | 0.0000258 | 0.00137807 | BM309263 | 315 | 14 | 39 | 35 | 0.84 | ion transport |
| 3264 | 4.912266836 | up | 0.00000479 | 0.000799682 | CD418459 | 653 | 83 | 116 | 71 | 1e−45 | protein biosynthesis |
| 3265 | 4.928167798 | up | 0.00000117 | 0.000704945 | AW756277 | | | | | | |
| 3266 | 4.930390428 | up | 0.00000231 | 0.000707618 | BQ628872 | 225 | 15 | 39 | 38 | 1.4 | proteolysis and peptidolysis |
| 3267 | 4.935413592 | up | 0.00000252 | 0.000712455 | BF069226 | 457 | 50 | 110 | 45 | 9e−15 | |
| 3268 | 4.935469101 | up | 0.000657818 | 0.007973945 | CD418606 | 291 | 15 | 58 | 25 | 5.4 | |
| 3269 | 4.938640424 | up | 0.0000186 | 0.00122629 | AW349391 | 767 | 131 | 164 | 79 | 8e−70 | response to cold |
| 3270 | 4.949177377 | up | 0.0000291 | 0.001465395 | CD401290 | 657 | 86 | 122 | 70 | 3e−45 | protein biosynthesis |
| 3271 | 4.9517402 | up | 0.000118372 | 0.002883786 | BM095037 | 514 | 41 | 94 | 43 | 4e−18 | |
| 3272 | 4.955514188 | up | 0.0000084 | 0.000900892 | BU548364 | 580 | 54 | 63 | 85 | 4e−29 | mitochondrial electron transport\, |
| | | | | | | | | | | | succinate to ubiquinone |
| 3273 | 4.962823503 | up | 0.000038 | 0.001660614 | BU551245 | 538 | 9 | 19 | 47 | 5.1 | protein amino acid glycosylation |
| 3274 | 4.963634129 | up | 0.00000708 | 0.000873658 | BE058913 | 418 | 53 | 83 | 63 | 1e−24 | starch catabolism |
| 3275 | 4.996612985 | up | 0.0000018 | 0.000704945 | BI971289 | 412 | 11 | 17 | 64 | 0.58 | |
| 3276 | 5.004816151 | up | 0.00000802 | 0.000899116 | CD409753 | 562 | 86 | 92 | 93 | 4e−46 | protein biosynthesis |
| 3277 | 5.014237376 | up | 0.000517593 | 0.006828742 | U27347.1 | | | | | | Pip1 protein |
| 3278 | 5.025362155 | up | 0.000113048 | 0.002817883 | AW164216 | 638 | 165 | 210 | 78 | 4e−99 | carbohydrate metabolism |
| 3279 | 5.031879888 | up | 0.00000126 | 0.000704945 | BG237280 | 231 | 14 | 35 | 40 | 7.1 | |
| 3280 | 5.044298541 | up | 0.00012702 | 0.002991688 | AF243182.1 | | | | | | beta-ketoacyl-ACP synthetase I /// |
| | | | | | | | | | | | beta-ketoacyl-ACP synthetase I-2 |
| 3281 | 5.049625692 | up | 0.000408704 | 0.005867922 | AW759068 | 562 | 40 | 51 | 78 | 8e−18 | protein folding |
| 3282 | 5.051242849 | up | 0.0000319 | 0.001527864 | CA801419 | 745 | 90 | 212 | 42 | 2e−41 | |
| 3283 | 5.069570727 | up | 0.000265169 | 0.004546066 | BE020100 | 266 | 15 | 62 | 24 | 7.2 | regulation of transcription |
| 3284 | 5.078067912 | up | 0.0000163 | 0.001156628 | CF809006 | 556 | 140 | 164 | 85 | 2e−81 | protein biosynthesis |
| 3285 | 5.078967454 | up | 0.00000198 | 0.000707618 | BE805933 | 569 | 62 | 200 | 31 | 3e−23 | |
| 3286 | 5.082675047 | up | 0.00009 | 0.002523425 | CD396846 | 711 | 70 | 145 | 48 | 3e−35 | response to pathogenic fungi |
| 3287 | 5.094255564 | up | 0.00000222 | 0.000707618 | CD416961 | 573 | 43 | 62 | 69 | 8e−18 | protein biosynthesis |
| 3288 | 5.153959667 | up | 0.00000187 | 0.000704945 | CD410399 | 662 | 135 | 142 | 95 | 2e−73 | protein biosynthesis |
| 3289 | 5.162541073 | up | 0.0000239 | 0.00133979 | BE020613 | 410 | 108 | 139 | 77 | 1e−56 | |
| 3290 | 5.184549685 | up | 0.00000397 | 0.000767688 | CD399598 | 619 | 102 | 132 | 77 | 9e−55 | |
| 3291 | 5.212446527 | up | 0.000000169 | 0.00067462 | BU547039 | 644 | 125 | 142 | 88 | 1e−66 | selenocysteine incorporation |
| 3292 | 5.21596179 | up | 0.000000318 | 0.00067462 | BU765471 | 420 | 55 | 64 | 85 | 2e−27 | transcription |
| 3293 | 5.221264074 | up | 0.00000504 | 0.000799682 | CD412496 | 495 | 67 | 78 | 85 | 7e−32 | |
| 3294 | 5.222322895 | up | 0.00000929 | 0.000924406 | CF806907 | 696 | 193 | 232 | 83 | e−113 | |
| 3295 | 5.244510263 | up | 0.0000199 | 0.001255458 | BI785205 | 563 | 57 | 66 | 86 | 8e−31 | protein biosynthesis |
| 3296 | 5.26002018 | up | 0.0000389 | 0.001688181 | BI968208 | 781 | 144 | 180 | 80 | 2e−78 | flavonoid biosynthesis |
| 3297 | 5.289833383 | up | 0.00000711 | 0.000873658 | BG508085 | 252 | 14 | 39 | 35 | 4.3 | response to oxidative stress |
| 3298 | 5.298104807 | up | 0.00000235 | 0.000707618 | CF808052 | 560 | 117 | 150 | 78 | 3e−61 | protein biosynthesis |
| 3299 | 5.318139798 | up | 0.000000449 | 0.00067462 | BM892707 | 565 | 44 | 66 | 66 | 3e−20 | |
| 3300 | 5.319601168 | up | 0.00000278 | 0.000720842 | BI942376 | 445 | 132 | 146 | 90 | 1e−70 | |
| 3301 | 5.330414731 | up | 0.000013 | 0.001078083 | BU547120 | 613 | 61 | 84 | 72 | 3e−28 | |
| 3302 | 5.343331832 | up | 0.00000595 | 0.000830526 | CD390794 | 618 | 43 | 135 | 31 | 7e−10 | |
| 3303 | 5.35005145 | up | 0.00000835 | 0.000900892 | CF806563 | 700 | 171 | 216 | 79 | 7e−94 | protein biosynthesis |
| 3304 | 5.350806283 | up | 0.00000103 | 0.000704945 | BU091656 | 563 | 124 | 183 | 67 | 4e−71 | |
| 3305 | 5.369140264 | up | 0.00000137 | 0.000704945 | CD417849 | 603 | 35 | 67 | 52 | 3e−14 | DNA repair |
| 3306 | 5.39579344 | up | 0.000247786 | 0.004383337 | CD395628 | 528 | 63 | 77 | 81 | 1e−31 | response to wounding |
| 3307 | 5.40689858 | up | 0.0000524 | 0.001952406 | BQ611306 | 356 | 14 | 29 | 48 | 2.5 | |
| 3308 | 5.443670108 | up | 0.000000502 | 0.00067462 | BI968832 | 697 | 101 | 197 | 51 | 3e−54 | |
| 3309 | 5.482053372 | up | 0.004540143 | 0.027709387 | BU084101 | 347 | 19 | 24 | 79 | 4e−07 | response to light |
| 3310 | 5.501574831 | up | 0.00000804 | 0.000899116 | CD405473 | 665 | 131 | 163 | 80 | 9e−72 | protein biosynthesis |
| 3311 | 5.504194933 | up | 0.0000386 | 0.001678137 | CD392557 | 429 | 29 | 58 | 50 | 3e−11 | |
| 3312 | 5.505432543 | up | 0.000761798 | 0.008770408 | AI442316 | 379 | 26 | 37 | 70 | 4e−13 | G-protein coupled receptor protein |
| | | | | | | | | | | | signaling pathway |
| 3313 | 5.530310978 | up | 0.00000122 | 0.000704945 | BF070149 | 434 | 49 | 53 | 92 | 2e−25 | mitochondrial electron transport\, |
| | | | | | | | | | | | succinate to ubiquinone |
| 3314 | 5.571845182 | up | 0.00000223 | 0.000707618 | CD408098 | 296 | 9 | 16 | 56 | 1.9 | |
| 3315 | 5.613350533 | up | 0.00000255 | 0.000712455 | CD416617 | 647 | 70 | 146 | 47 | 3e−28 | |
| 3316 | 5.630101767 | up | 0.00000229 | 0.000707618 | CD407962 | 433 | 75 | 92 | 81 | 5e−39 | DNA metabolism |
| 3317 | 5.634934418 | up | 0.000591477 | 0.007455707 | BE609302 | 614 | 36 | 46 | 78 | 1e−15 | regulation of transcription\, DNA- |
| | | | | | | | | | | | dependent |
| 3318 | 5.637477913 | up | 0.0000856 | 0.002466308 | BI786013 | 576 | 52 | 80 | 65 | 9e−27 | |
| 3319 | 5.650171873 | up | 0.00000441 | 0.000778398 | BM522378 | 577 | 125 | 192 | 65 | 2e−71 | multidrug transport |
| 3320 | 5.657722876 | up | 0.0000277 | 0.001430061 | CD403542 | 628 | 14 | 30 | 46 | 8.7 | |
| 3321 | 5.687273941 | up | 0.00000951 | 0.000930683 | CA935467 | 432 | 31 | 51 | 60 | 7e−11 | protein biosynthesis |
| 3322 | 5.696244056 | up | 0.001558574 | 0.013681257 | BI974374 | 535 | 107 | 155 | 69 | 6e−56 | response to oxidative stress |
| 3323 | 5.696339087 | up | 0.00000437 | 0.000778181 | BG511612 | 340 | 15 | 17 | 88 | 0.026 | threonine catabolism |
| 3324 | 5.716724964 | up | 0.0000518 | 0.001936725 | BU080867 | 350 | 28 | 40 | 70 | 7e−14 | |
| 3325 | 5.73106544 | up | 0.000000606 | 0.000677501 | CF807322 | 749 | 200 | 251 | 79 | e−108 | |
| 3326 | 5.766206773 | up | 0.00000148 | 0.000704945 | AW350912 | 511 | 52 | 64 | 81 | 1e−25 | |
| 3327 | 5.77261722 | up | 0.0000528 | 0.001953259 | CD405901 | 351 | 8 | 21 | 38 | 2.4 | |
| 3328 | 5.789524585 | up | 0.00000694 | 0.000870238 | BI969187 | 795 | 15 | 36 | 41 | 2.0 | |
| 3329 | 5.813458541 | up | 0.00000593 | 0.000830526 | BU926524 | 341 | 22 | 24 | 91 | 4e−07 | immune response |
| 3330 | 5.877791106 | up | 0.002090251 | 0.01658007 | BI945611 | 549 | 50 | 64 | 78 | 3e−23 | response to oxidative stress |
| 3331 | 5.879127912 | up | 0.000727422 | 0.008521103 | BU547877 | 670 | 128 | 182 | 70 | 2e−77 | carbohydrate metabolism |
| 3332 | 5.90078142 | up | 0.000000218 | 0.00067462 | AW132322 | 548 | 59 | 74 | 79 | 4e−27 | |
| 3333 | 6.044136877 | up | 0.000000538 | 0.00067462 | CD393006 | 672 | 38 | 84 | 45 | 6e−13 | |
| 3334 | 6.190062904 | up | 0.000000622 | 0.000677501 | L28003.1 | | | | | | TGACG-motif binding factor |
| 3335 | 6.193409086 | up | 0.000198219 | 0.00382741 | CD406004 | 667 | 116 | 156 | 74 | 2e−68 | carbohydrate metabolism |
| 3336 | 6.201371518 | up | 0.00000109 | 0.000704945 | BU579272 | 582 | 107 | 172 | 62 | 3e−57 | |
| 3337 | 6.216187271 | up | 0.000391772 | 0.005724982 | AW598355 | 555 | 93 | 105 | 88 | 4e−49 | response to wounding |
| 3338 | 6.224450869 | up | 0.000000668 | 0.000677501 | CF807322 | 749 | 200 | 251 | 79 | e−108 | |
| 3339 | 6.286013619 | up | 0.002086462 | 0.016557 | BQ629660 | 604 | 16 | 31 | 51 | 0.018 | |
| 3340 | 6.312377728 | up | 0.000875807 | 0.009526488 | BI974591 | 521 | 123 | 172 | 71 | 1e−69 | response to oxidetive stress |
| 3341 | 6.3442658 | up | 0.0000263 | 0.001384805 | CD401987 | 598 | 57 | 94 | 60 | 2e−33 | |
| 3342 | 6.353093316 | up | 0.0000542 | 0.001976427 | BE022293 | 392 | 12 | 29 | 41 | 3.4 | cellulose biosynthesis |
| 3343 | 6.381510891 | up | 0.0000127 | 0.001064794 | BI699134 | 459 | 13 | 28 | 46 | 1.3 | |
| 3344 | 6.420799936 | up | 0.001701697 | 0.014481366 | CD486940 | 579 | 26 | 30 | 86 | 1e−08 | ciliary/flagellar motility |
| 3345 | 6.495700091 | up | 0.000000979 | 0.000704945 | BE190863 | 536 | 120 | 191 | 62 | 1e−58 | response to light |
| 3346 | 6.511534344 | up | 0.00000932 | 0.000924406 | BI424359 | 427 | 46 | 61 | 75 | 2e−19 | serine O-acetyltransferase 1, |
| | | | | | | | | | | | cysteine biosynthesis from serine |
| 3347 | 6.624305642 | up | 0.002047467 | 0.016384686 | CD394773 | 524 | 31 | 52 | 59 | 2e−11 | regulation of transcription |
| 3348 | 6.645058245 | up | 0.000000953 | 0.000704945 | CF808765 | 573 | 171 | 190 | 90 | 2e−97 | Enolase, response to cold |
| 3349 | 6.713429939 | up | 0.00000619 | 0.000832282 | CD398253 | 274 | 10 | 25 | 40 | 5.5 | |
| 3350 | 6.716421946 | up | 0.0000108 | 0.000981585 | BM520465 | 573 | 12 | 21 | 57 | 1.2 | |
| 3351 | 6.738964047 | up | 0.0000249 | 0.001364741 | BI785748 | 577 | 163 | 187 | 87 | 1e−99 | |
| 3352 | 6.778670364 | up | 0.00000137 | 0.000704945 | AI416867 | 499 | 80 | 90 | 88 | 9e−38 | |
| 3353 | 6.80628125 | up | 0.00000752 | 0.000889839 | CD393835 | | | | | | |
| 3354 | 6.941097409 | up | 0.000000211 | 0.00067462 | CD395848 | 444 | 45 | 86 | 52 | 4e−24 | transport |
| 3355 | 6.993282629 | up | 0.005605758 | 0.031941238 | AW569963 | 393 | 25 | 43 | 58 | 2e−08 | |
| 3356 | 7.037442069 | up | 0.000000894 | 0.000704945 | AW309388 | 587 | 94 | 137 | 68 | 4e−51 | D-ribose metabolism |
| 3357 | 7.062536836 | up | 0.000000264 | 0.00067462 | CA853912 | 550 | 91 | 172 | 52 | 3e−46 | ATP-dependent proteolysis |
| 3358 | 7.094899646 | up | 0.001603002 | 0.013905915 | BE608612 | 334 | 11 | 23 | 47 | 0.22 | DNA repair |
| 3359 | 7.211518639 | up | 0.00000108 | 0.000704945 | BQ453230 | 517 | 75 | 97 | 77 | 3e−40 | |
| 3360 | 7.27901913 | up | 0.00000128 | 0.000704945 | CD391958 | 581 | 81 | 93 | 87 | 6e−42 | RNA methylation |
| 3361 | 7.313422507 | up | 0.000000288 | 0.00067462 | CD408023 | 379 | 14 | 57 | 24 | 0.46 | |
| 3362 | 7.333114784 | up | 0.00000184 | 0.000704945 | CF807999 | 543 | 124 | 181 | 68 | 1e−69 | electron transport |
| 3363 | 7.351496924 | up | 0.00000409 | 0.000768376 | CD408636 | 378 | 19 | 58 | 32 | 0.019 | |
| 3364 | 7.418119354 | up | 0.0000931 | 0.002553736 | CA803057 | 346 | 37 | 41 | 90 | 3e−15 | |
| 3365 | 7.447529321 | up | 0.0000643 | 0.002135499 | BM309451 | 563 | 13 | 32 | 40 | 5.5 | |
| 3366 | 7.455518172 | up | 0.002569993 | 0.018973054 | CF807853 | 237 | 16 | 47 | 34 | 1.1 | lipid transport |
| 3367 | 7.489390838 | up | 0.003657503 | 0.023884642 | BI967997 | 788 | 161 | 199 | 80 | 3e−89 | biological_process unknown |
| 3368 | 7.57580059 | up | 0.0000018 | 0.000704945 | BU549963 | 631 | 13 | 34 | 38 | 4.0 | adaxial/abaxial pattern formation |
| 3369 | 7.818689828 | up | 0.002886879 | 0.020497672 | AW570011 | 478 | 125 | 159 | 78 | 3e−72 | protein folding |
| 3370 | 7.873257673 | up | 6.21E−08 | 0.00067462 | BQ252648 | 514 | 69 | 94 | 73 | 9e−36 | ATP-dependent proteolysis |
| 3371 | 7.883045549 | up | 0.000199097 | 0.003834522 | BG650202 | 280 | 15 | 58 | 25 | 7.1 | |
| 3372 | 7.902128667 | up | 0.0000017 | 0.000704945 | CD412630 | 736 | 57 | 72 | 79 | 2e−28 | early light-induced protein, |
| | | | | | | | | | | | photosynthesis light harvesting |
| 3373 | 8.057537471 | up | 0.0000391 | 0.001692808 | AW704742 | 351 | 90 | 117 | 76 | 8e−52 | tip growth |
| 3374 | 8.175316527 | up | 0.000000586 | 0.000677501 | BM270707 | 339 | 21 | 30 | 70 | 2e−06 | transport |
| 3375 | 8.250243859 | up | 0.0000178 | 0.001214142 | AW568275 | 367 | 15 | 38 | 39 | 1.6 | |
| 3376 | 8.261267374 | up | 0.000000676 | 0.000677501 | BF009846 | 294 | 31 | 97 | 31 | 1e−09 | transport |
| 3377 | 8.383278342 | up | 0.001427398 | 0.012906284 | BU550606 | 601 | 122 | 147 | 82 | 4e−62 | response to heat |
| 3378 | 8.439748544 | up | 0.00000177 | 0.000704945 | AW734913 | 269 | 13 | 43 | 30 | 4.2 | |
| 3379 | 8.475628006 | up | 0.0000213 | 0.001287717 | U21722.1 | | | | | | Hsp23.9 |
| 3380 | 8.649647578 | up | 0.000000501 | 0.00067462 | BM892671 | 544 | 85 | 114 | 74 | 8e−51 | |
| 3381 | 8.704688442 | up | 0.002154847 | 0.017002812 | AW569879 | 511 | 63 | 87 | 72 | 1e−32 | response to wounding |
| 3382 | 8.721818183 | up | 8.58E−08 | 0.00067462 | BG047431 | 218 | 13 | 31 | 41 | 0.10 | DNA transposition |
| 3383 | 8.768108104 | up | 0.000000918 | 0.000704945 | AW832198 | 421 | 53 | 63 | 84 | 2e−25 | RNA methylation |
| 3384 | 8.775621733 | up | 0.000835473 | 0.009252189 | BI968043 | 268 | 12 | 31 | 38 | 0.65 | |
| 3385 | 8.792603282 | up | 0.00000888 | 0.000915272 | L28003.1 | | | | | | TGACG-motif-binding factor /// |
| | | | | | | | | | | | TGACG-motif binding factor |
| 3386 | 8.824725816 | up | 0.000928359 | 0.009892151 | CD403581 | 541 | 44 | 70 | 62 | 5e−22 | |
| 3387 | 8.846511326 | up | 0.0000011 | 0.000704945 | CA802902 | 790 | 200 | 243 | 82 | e−110 | |
| 3388 | 8.875685323 | up | 0.00000121 | 0.000704945 | CA802301 | 683 | 207 | 220 | 94 | e−118 | |
| 3389 | 8.965500274 | up | 0.000000189 | 0.00067462 | CA819374 | 491 | 54 | 75 | 72 | 3e−26 | |
| 3390 | 9.399499838 | up | 0.006210167 | 0.034275749 | BE658252 | 713 | 39 | 49 | 79 | 2e−20 | |
| 3391 | 9.593199093 | up | 0.000047 | 0.001823768 | CF806186 | 568 | 174 | 189 | 92 | 3e−98 | selenocysteine incorporation |
| 3392 | 9.69522791 | up | 0.000101759 | 0.002669158 | BM892178 | 437 | 113 | 144 | 78 | 3e−64 | tip growth |
| 3393 | 9.786036338 | up | 0.000000528 | 0.00067462 | CD404896 | 652 | 121 | 162 | 74 | 2e−71 | |
| 3394 | 9.880948687 | up | 0.001431707 | 0.012931513 | AW395303 | 531 | 35 | 51 | 68 | 8e−19 | transcription |
| 3395 | 10.43572903 | up | 0.000000114 | 0.00067462 | CD416450 | 336 | 23 | 54 | 42 | 7e−11 | |
| 3396 | 10.45849749 | up | 0.000130685 | 0.00303449 | BE657436 | 777 | 95 | 147 | 64 | 1e−48 | |
| 3397 | 10.64157538 | up | 0.001046166 | 0.010710535 | BU550606 | 601 | 122 | 147 | 82 | 4e−62 | response to heat |
| 3398 | 10.99067149 | up | 0.000000638 | 0.000677501 | AW152956 | 575 | 98 | 154 | 63 | 7e−54 | metabolism |
| 3399 | 11.22980765 | up | 0.000812331 | 0.009088345 | CF808111 | 540 | 112 | 139 | 80 | 7e−58 | response to heat |
| 3400 | 11.73076193 | up | 0.001240042 | 0.011844351 | BM887146 | 546 | 127 | 182 | 69 | 6e−70 | response to wounding |
| 3401 | 11.83256274 | up | 0.0000411 | 0.001735199 | AI973750 | 421 | 14 | 44 | 31 | 1.3 | |
| 3402 | 12.01685092 | up | 0.000793808 | 0.008963767 | CF806638 | 616 | 122 | 162 | 75 | 1e−64 | |
| 3403 | 12.06592608 | up | 0.0000125 | 0.001058667 | CD417466 | 659 | 75 | 97 | 77 | 2e−38 | |
| 3404 | 12.17521797 | up | 0.00000108 | 0.000704945 | CD407204 | 712 | 165 | 177 | 93 | 3e−93 | |
| 3405 | 12.70888214 | up | 0.00000953 | 0.000930683 | U21722.1 | | | | | | |
| 3406 | 12.79143995 | up | 0.000000449 | 0.00067462 | CD398633 | 543 | 14 | 49 | 28 | 4.0 | |
| 3407 | 12.84285598 | up | 0.00000193 | 0.000707618 | BI945176 | 430 | 18 | 71 | 25 | 0.37 | Mo-molybdopterin cofactor |
| | | | | | | | | | | | biosynthesis |
| 3408 | 12.99408833 | up | 0.00000455 | 0.000784876 | AF243367.1 | | | | | | glutathione S-transferase GST 12 |
| 3409 | 13.55312591 | up | 2.63E−08 | 0.000495355 | BE802263 | 603 | 80 | 195 | 41 | 4e−39 | |
| 3410 | 13.72343106 | up | 0.005156454 | 0.030184979 | CF807875 | 644 | 107 | 155 | 69 | 3e−57 | |
| 3411 | 13.90789177 | up | 0.006007229 | 0.033489836 | AW620208 | 417 | 32 | 52 | 61 | 8e−11 | |
| 3412 | 14.39846457 | up | 0.0000794 | 0.002376825 | AW706122 | 301 | 13 | 45 | 28 | 0.50 | secondary cell wall biosynthesis |
| | | | | | | | | | | | (sensu Magnoliophyta) |
| 3413 | 14.72969424 | up | 0.000026 | 0.001380871 | BE802487 | 326 | 27 | 91 | 29 | 0.13 | cellulose biosynthesis |
| 3414 | 15.03655496 | up | 0.00000204 | 0.000707618 | BQ740754 | | | | | | |
| 3415 | 15.05925429 | up | 0.0000845 | 0.002456504 | CD394837 | 374 | 15 | 37 | 40 | 4.9 | |
| 3416 | 15.67105724 | up | 0.000004 | 0.000768376 | AW100946 | 419 | 10 | 27 | 37 | 3.0 | |
| 3417 | 16.39787909 | up | 0.007895448 | 0.040126925 | AW569992 | 391 | 88 | 129 | 68 | 8e−47 | |
| 3418 | 16.45163382 | up | 0.007103922 | 0.037460665 | AW568567 | 438 | 50 | 63 | 79 | 3e−24 | |
| 3419 | 16.46632398 | up | 0.00000189 | 0.000704945 | BQ740754 | | | | | | |
| 3420 | 16.96537578 | up | 0.00000988 | 0.000947882 | CF806823 | 692 | 101 | 214 | 47 | 4e−46 | Hsp23.9 |
| 3421 | 17.1995684 | up | 0.007326902 | 0.038170926 | AW569992 | 391 | 88 | 129 | 68 | 8e−47 | |
| 3422 | 17.56272328 | up | 0.00000118 | 0.000704945 | AW397732 | 373 | 33 | 54 | 61 | 3e−15 | electron transport |
| 3423 | 18.17890818 | up | 0.0000085 | 0.000900892 | CD401350 | 386 | 30 | 37 | 81 | 3e−12 | |
| 3424 | 19.02342162 | up | 0.005882957 | 0.033032029 | AW569786 | 372 | 43 | 53 | 81 | 2e−19 | |
| 3425 | 19.69829408 | up | 0.000000622 | 0.000677501 | CF809067 | 638 | 117 | 161 | 72 | 2e−61 | response to oxidative stress |
| 3426 | 20.30309891 | up | 0.00000506 | 0.000799682 | BI316016 | 216 | 17 | 43 | 39 | 1.1 | |
| 3427 | 21.84555273 | up | 0.005611879 | 0.03195174 | AW569992 | 391 | 88 | 129 | 68 | 8e−47 | |
| 3428 | 24.19441236 | up | 0.000000182 | 0.00067462 | X68702.1 | | | | | | alternative oxidase |
| 3429 | 25.54946501 | up | 0.000356982 | 0.005420878 | AW567704 | 355 | 19 | 51 | 37 | 0.51 | |
| 3430 | 26.09912328 | up | 0.000347562 | 0.005339087 | BU550058 | 631 | 84 | 132 | 63 | 5e−43 | |
| 3431 | 35.53754192 | up | 6.65E−09 | 0.000250191 | BG042849 | 407 | 45 | 116 | 38 | 1e−17 | |

The functional classification of the differentially expressed genes in soybean root under drought condition is summarized in Table 3, which shows the numbers of genes that are either up- or down-regulated in each category as defined by protein function.

TABLE 3
Functional Classification of drought responsive transcripts in
soybean root tissues:

| | | | Up + |
|---|---|---|---|
| | Up | Down | Down |
| Root tissue | regulated | regulated | regulated |
| Information Storage and Processing | 14 | 187 | 201 |
| Transcription | 23 | 147 | 170 |
| Metabolism | 96 | 619 | 715 |
| Amino Acid Metabolism | 28 | 132 | 160 |
| Carbohydrate Metabolism | 36 | 273 | 309 |
| Cellular Process and Signaling | 125 | 599 | 724 |
| Signal Transduction | 44 | 274 | 318 |
| Poorly Characterized | 109 | 574 | 683 |
| No Annotation | 409 | 2624 | 3033 |
| Total | 884 | 5429 | 6313 |

Genes or DNA fragments from the root tissues that show at least two fold difference in expression levels between those soybean plants grown under normal water condition and those under drought conditions are listed in Table 4. The designation of columns are similar to those of Table 2. The sequences of all DRGs listed in Tables 2 and 4 as identified by the PublicID are hereby incorporated by reference into this disclosure as if fully reproduced herein.

TABLE 4
List of Soybean Root genes or fragments with at least 2 fold
differential expression under normal or drought conditions

| Item No. | FC | Direction | P Value | adjPVal | Public ID | Query_Length | Iden_numero | Iden_denom | % identity | E-value | Description and possible Function |
|---|---|---|---|---|---|---|---|---|---|---|---|
| 3432 | 67.92858004 | down | 7.88E−08 | 0.0000076 | BQ081552 | | | | | | |
| 3433 | 60.66750493 | down | 9.7E−09 | 0.00000287 | AW349294 | 776 | 20 | 60 | 33 | 1.9 | |
| 3434 | 59.36962999 | down | 6.54E−10 | 0.00000078 | BI786195 | 570 | 53 | 93 | 56 | 4e−24 | carbohydrate metabolism |
| 3435 | 57.94699009 | down | 2.01E−09 | 0.00000146 | BI967267 | 736 | 57 | 157 | 36 | 4e−19 | |
| 3436 | 52.24208562 | down | 4.91E−10 | 0.000000745 | BM093908 | 247 | 9 | 20 | 45 | 9.2 | |
| 3437 | 48.72080456 | down | 1.15E−08 | 0.00000304 | BF008858 | 606 | 59 | 85 | 69 | 1e−35 | regulation of transcription\, DNA-1 dependent |
| 3438 | 43.92855339 | down | 1.65E−09 | 0.00000129 | AW278629 | 496 | 54 | 84 | 64 | 4e−32 | |
| 3439 | 43.00939257 | down | 9.02E−09 | 0.00000278 | AW309755 | 663 | 76 | 136 | 55 | 3e−34 | |
| 3440 | 42.26568355 | down | 8.7E−10 | 0.000000915 | CF920328 | 762 | 65 | 125 | 52 | 2e−27 | |
| 3441 | 41.090768 | down | 5.96E−09 | 0.00000221 | CD416703 | 592 | 41 | 84 | 48 | 3e−19 | |
| 3442 | 40.55123135 | down | 2.2E−10 | 0.000000589 | BI700284 | 381 | 13 | 32 | 40 | 1.1 | protein amino acid phosphorylation |
| 3443 | 38.9009012 | down | 2.25E−09 | 0.00000146 | BQ452830 | 450 | 14 | 31 | 45 | 6.0 | proteolysis and peptidolysis |
| 3444 | 36.93987903 | down | 3.75E−10 | 0.000000672 | CF807894 | 489 | 13 | 26 | 50 | 3.2 | 28 kDa protein |
| 3445 | 35.41653907 | down | 7.85E−10 | 0.000000894 | BI497973 | 453 | 13 | 26 | 50 | 2.7 | |
| 3446 | 35.34370608 | down | 3.51E−09 | 0.00000183 | AF019116.1 | | | | | | peroxidase |
| 3447 | 35.27434892 | down | 5.49E−10 | 0.000000753 | AW201640 | 627 | 13 | 26 | 50 | 5.1 | 28 kDa protein |
| 3448 | 34.60081864 | down | 1.8E−10 | 0.000000589 | BE658397 | 613 | 13 | 26 | 50 | 4.9 | 28 kDa protein |
| 3449 | 34.01804229 | down | 0.00000067 | 0.0000267 | AF127110.1 | | | | | | ripening related protein |
| 3450 | 33.35553918 | down | 1.56E−08 | 0.00000353 | BI969869 | 572 | 40 | 53 | 75 | 2e−17 | |
| 3451 | 32.94109633 | down | 2.78E−10 | 0.000000616 | CA850542 | 367 | 9 | 13 | 69 | 3.6 | carbohydrate biosynthesis |
| 3452 | 32.60301077 | down | 0.000000879 | 0.0000311 | CK606455 | 809 | 122 | 240 | 50 | 2e−62 | copper amino oxidase |
| 3453 | 32.4628378 | down | 0.000012 | 0.000158814 | CK606317 | 701 | 129 | 211 | 61 | 4e−70 | transport |
| 3454 | 32.00277349 | down | 3.22E−08 | 0.00000473 | CK606380 | 390 | 45 | 104 | 43 | 1e−19 | ripening related protein |
| 3455 | 31.33659335 | down | 0.0000713 | 0.000542103 | BU764905 | 336 | 8 | 28 | 28 | 3.2 | |
| 3456 | 30.61632715 | down | 2.38E−08 | 0.00000412 | CK605560 | 600 | 31 | 125 | 24 | 0.95 | |
| 3457 | 30.37050122 | down | 4.58E−08 | 0.00000565 | AW309773 | 501 | 12 | 37 | 32 | 0.40 | |
| 3458 | 30.1134729 | down | 0.00000754 | 0.000116381 | AW309597 | 663 | 76 | 108 | 70 | 2e−44 | antibiotic biosynthesis |
| 3459 | 29.72082191 | down | 6.53E−09 | 0.00000225 | CD391892 | | | | | | |
| 3460 | 29.26269625 | down | 1.16E−08 | 0.00000304 | AW394497 | 258 | 12 | 23 | 52 | 9.4 | |
| 3461 | 29.00333393 | down | 1.07E−08 | 0.00000293 | BQ612452 | 590 | 68 | 118 | 57 | 2e−34 | |
| 3462 | 28.86201166 | down | 5.8E−09 | 0.00000221 | CA852384 | 385 | 11 | 20 | 55 | 3.1 | |
| 3463 | 28.60673423 | down | 0.00000128 | 0.0000382 | AW423744 | 384 | 16 | 29 | 55 | 7e−05 | transport |
| 3464 | 27.55650594 | down | 0.0000192 | 0.00021876 | AW349799 | 528 | 38 | 54 | 70 | 8e−17 | electron transport |
| 3465 | 27.51051381 | down | 0.00000027 | 0.000015 | BM567976 | 366 | 11 | 27 | 40 | 0.72 | |
| 3466 | 27.49404046 | down | 0.000000026 | 0.00000426 | BM139635 | 304 | 41 | 75 | 54 | 1e−21 | |
| 3467 | 27.45589948 | down | 0.0000868 | 0.000623781 | AW310071 | 696 | 72 | 173 | 41 | 1e−34 | |
| 3468 | 27.4086228 | down | 6.33E−08 | 0.00000688 | AW311637 | 212 | 15 | 37 | 40 | 0.077 | |
| 3469 | 27.26846545 | down | 0.000000748 | 0.0000285 | BQ611386 | 255 | 22 | 26 | 84 | 4e−08 | |
| 3470 | 26.96721085 | down | 4.42E−08 | 0.00000558 | BQ613046 | 345 | 38 | 49 | 77 | 5e−17 | transport |
| 3471 | 26.71063747 | down | 2.46E−08 | 0.00000414 | CA800035 | 383 | 27 | 109 | 24 | 0.63 | protein amino acid phosphorylation |
| 3472 | 26.11692239 | down | 2.69E−10 | 0.000000616 | BM522941 | 230 | 14 | 44 | 31 | 2.4 | circadian rhythm |
| 3473 | 25.78792994 | down | 5.33E−10 | 0.000000753 | BM107847 | 764 | 102 | 152 | 67 | 2e−56 | |
| 3474 | 25.26617883 | down | 1.82E−08 | 0.00000368 | BU765206 | 424 | 26 | 58 | 44 | 2e−08 | |
| 3475 | 24.46951801 | down | 0.000843434 | 0.003221458 | BM525407 | 420 | 16 | 37 | 43 | 1.3 | |
| 3476 | 24.37691536 | down | 5.42E−09 | 0.00000216 | AW278220 | 542 | 137 | 159 | 86 | 1e−72 | glycolysis |
| 3477 | 24.24102307 | down | 0.00000202 | 0.0000512 | AW309863 | 679 | 38 | 83 | 45 | 8e−15 | |
| 3478 | 23.67922488 | down | 0.00000498 | 0.0000907 | BI967614 | | | | | | |
| 3479 | 23.55167488 | down | 3.16E−10 | 0.000000626 | BU547363 | 627 | 80 | 144 | 55 | 2e−40 | electron transport |
| 3480 | 23.45297193 | down | 1.54E−09 | 0.00000126 | BE658385 | 582 | 55 | 124 | 44 | 4e−21 | cell-cell signaling |
| 3481 | 23.35667663 | down | 2.01E−08 | 0.0000038 | CF922276 | 724 | 74 | 134 | 55 | 7e−31 | |
| 3482 | 22.61502809 | down | 8.86E−09 | 0.00000276 | AW317179 | 529 | 61 | 83 | 73 | 2e−29 | weakly similar to NP_922657.1 Oryza |
| | | | | | | | | | | | sativa (japonica cultivar-group) putative |
| | | | | | | | | | | | lipid transfer protein |
| | | | | | | | | | | | (OSJNBa0015J15.12), mRNA |
| 3483 | 22.54012102 | down | 0.0000301 | 0.000298682 | BQ612924 | 557 | 70 | 137 | 51 | 3e−36 | |
| 3484 | 22.36614196 | down | 9.21E−10 | 0.000000936 | BQ612015 | 429 | 53 | 70 | 75 | 6e−28 | |
| 3485 | 21.9911512 | down | 1.34E−08 | 0.00000331 | BE821618 | 627 | 34 | 72 | 47 | 2e−12 | |
| 3486 | 21.7857757 | down | 2.04E−08 | 0.00000381 | BI967765 | 787 | 151 | 209 | 72 | 2e−79 | water transport |
| 3487 | 21.71955277 | down | 2.47E−08 | 0.00000414 | BM139646 | 343 | 13 | 26 | 50 | 1.1 | cation transport |
| 3488 | 21.42634692 | down | 0.000000372 | 0.0000185 | AI736435 | 583 | 60 | 189 | 31 | 7e−22 | |
| 3489 | 21.39514235 | down | 0.0000276 | 0.000281203 | AW349743 | 561 | 44 | 84 | 52 | 9e−24 | |
| 3490 | 21.27248481 | down | 1.07E−08 | 0.00000293 | AW310765 | 472 | 18 | 23 | 78 | 2e−04 | response to oxidative stress |
| 3491 | 21.21942388 | down | 2.78E−08 | 0.00000439 | CD390793 | 493 | 11 | 39 | 28 | 2.5 | |
| 3492 | 21.15249313 | down | 0.000942122 | 0.003499603 | BQ453202 | 543 | 53 | 109 | 48 | 3e−27 | |
| 3493 | 21.09769514 | down | 3.09E−09 | 0.00000175 | CD486940 | 579 | 26 | 30 | 86 | 1e−08 | ciliary/flagellar motility |
| 3494 | 20.77261006 | down | 0.000147898 | 0.000907681 | AF135485.1 | | | | | | Cytochrome P450 monooxygenaseCYP93D1 |
| | | | | | | | | | | | (CYP93E1) |
| 3495 | 20.60627906 | down | 0.000214282 | 0.001186866 | BG046570 | 423 | 25 | 83 | 30 | 0.80 | protein amino acid phosphorylation |
| 3496 | 20.43032084 | down | 6.23E−09 | 0.00000221 | U03860.1 | | | | | | cytokinin induced message |
| 3497 | 20.26910545 | down | 7.96E−09 | 0.00000253 | AW306565 | 502 | 9 | 18 | 50 | 9.9 | cytidine metabolism |
| 3498 | 20.13147871 | down | 3.84E−08 | 0.00000517 | CF807325 | 604 | 104 | 135 | 77 | 1e−54 | |
| 3499 | 20.10935932 | down | 1.05E−08 | 0.00000293 | BI700784 | 369 | 13 | 16 | 81 | 0.20 | response to oxidative stress |
| 3500 | 20.08348033 | down | 0.000000314 | 0.0000167 | BQ473745 | 348 | 13 | 39 | 33 | 2.5 | carbohydrate metabolism |
| 3501 | 20.02762434 | down | 1.07E−10 | 0.000000589 | BE023458 | 411 | 13 | 37 | 35 | 3.8 | response to auxin stimulus |
| 3502 | 20.01937843 | down | 1.92E−08 | 0.00000376 | CA852004 | 330 | 26 | 41 | 63 | 1e−10 | response to oxidative stress |
| 3503 | 19.998626 | down | 0.00000634 | 0.000105028 | BM523459 | 589 | 44 | 128 | 34 | 4e−17 | |
| 3504 | 19.95203659 | down | 0.00000245 | 0.0000582 | CK605851 | 294 | 22 | 60 | 36 | 0.009 | Lipoxygenase L-5 (vlxB) |
| 3505 | 19.88257123 | down | 0.0000143 | 0.000179941 | CF807748 | 285 | 10 | 24 | 41 | 4.1 | electron transport |
| 3506 | 19.77345189 | down | 0.000000779 | 0.0000291 | BE659369 | 787 | 74 | 138 | 53 | 6e−36 | response to oxidative stress |
| 3507 | 19.76176836 | down | 3.18E−08 | 0.00000472 | CA802118 | 451 | 54 | 64 | 84 | 5e−28 | |
| 3508 | 19.57192115 | down | 0.000000494 | 0.0000221 | BI970492 | 787 | 77 | 167 | 46 | 2e−32 | response to oxidative stress |
| 3509 | 19.5449823 | down | 2.02E−10 | 0.000000589 | BI700784 | 369 | 13 | 16 | 81 | 0.20 | response to oxidative stress |
| 3510 | 19.46390145 | down | 0.00000698 | 0.000111102 | AW185750 | 383 | 9 | 26 | 34 | 5.3 | Myo-inositol-3-phosphate synthase |
| | | | | | | | | | | | (MIPS), regulation of transcription\, DNA |
| | | | | | | | | | | | dependent |
| 3511 | 19.30489856 | down | 0.000000231 | 0.000014 | AW278125 | 592 | 79 | 188 | 42 | 2e−37 | |
| 3512 | 19.233447 | down | 2.35E−09 | 0.0000015 | BE347158 | 413 | 15 | 31 | 48 | 0.16 | lipid transport |
| 3513 | 19.19469348 | down | 1.58E−10 | 0.000000589 | BI969249 | 681 | 73 | 130 | 56 | 2e−34 | response to oxidative stress |
| 3514 | 19.13663121 | down | 0.00000002 | 0.0000038 | BU551320 | 537 | 37 | 57 | 64 | 7e−16 | transport |
| 3515 | 19.10132148 | down | 2.18E−09 | 0.00000146 | BI702248 | 441 | 76 | 115 | 66 | 2e−36 | |
| 3516 | 18.88507557 | down | 7.79E−09 | 0.0000025 | BI969594 | 766 | 108 | 193 | 55 | 1e−57 | |
| 3517 | 18.84514137 | down | 6.16E−08 | 0.00000682 | CD398582 | 602 | 121 | 171 | 70 | 2e−68 | |
| 3518 | 18.83948846 | down | 5.79E−08 | 0.0000066 | CD397515 | 682 | 60 | 97 | 61 | 3e−30 | |
| 3519 | 18.58241462 | down | 0.00035195 | 0.001700018 | AF135485.1 | | | | | | Cytochrome P450 |
| | | | | | | | | | | | monooxygenaseCYP93D1 (CYP93E1) |
| 3520 | 18.49682292 | down | 2.82E−09 | 0.00000167 | AW350788 | 507 | 67 | 95 | 70 | 1e−34 | response to oxidative stress |
| 3521 | 18.41145287 | down | 5.95E−09 | 0.00000221 | CA800035 | 383 | 27 | 109 | 24 | 0.63 | protein amino acid phosphorylation |
| 3522 | 18.36712463 | down | 0.000000798 | 0.0000294 | CD410143 | 676 | 155 | 159 | 97 | 2e−89 | microtubule-based movement |
| 3523 | 18.32210157 | down | 0.0000292 | 0.000291836 | CD403938 | 671 | 63 | 138 | 45 | 6e−34 | metabolism |
| 3524 | 17.91556388 | down | 1.77E−08 | 0.00000368 | BQ612737 | 377 | 12 | 31 | 38 | 1.7 | |
| 3525 | 17.9136351 | down | 2.03E−10 | 0.000000589 | BM139472 | 424 | 27 | 48 | 56 | 3e−08 | |
| 3526 | 13.16552213 | down | 0.00000104 | 0.0000342 | AW101606 | 241 | 13 | 24 | 54 | 1.4 | peroxidase precursor |
| 3527 | 13.15040651 | down | 0.00000354 | 0.0000729 | CF808078 | 553 | 128 | 182 | 70 | 1e−75 | |
| 3528 | 13.11789666 | down | 0.000000152 | 0.000011 | CD393884 | 415 | 19 | 42 | 45 | 0.024 | |
| 3529 | 13.11184801 | down | 0.00000913 | 0.000131604 | AW234900 | 607 | 122 | 202 | 60 | 8e−69 | transport |
| 3530 | 12.97886969 | down | 4.18E−08 | 0.00000536 | CA785639 | 422 | 15 | 26 | 57 | 0.001 | |
| 3531 | 12.88290172 | down | 0.000164151 | 0.000978196 | AW309848 | 593 | 51 | 102 | 50 | 4e−23 | |
| 3532 | 12.86065189 | down | 8.97E−08 | 0.00000811 | CD402591 | 502 | 55 | 86 | 63 | 5e−25 | |
| 3533 | 12.82879914 | down | 5.53E−08 | 0.00000644 | BG156156 | 451 | 22 | 59 | 37 | 0.064 | |
| 3534 | 12.80954458 | down | 2.16E−09 | 0.00000146 | BE821923 | 543 | 41 | 55 | 74 | 3e−19 | proteolysis and peptidolysis |
| 3535 | 12.80155681 | down | 0.00000014 | 0.0000106 | BU081783 | 488 | 28 | 33 | 84 | 5e−11 | electron transport |
| 3536 | 12.79587174 | down | 1.27E−08 | 0.00000319 | BQ627716 | 346 | 11 | 24 | 45 | 2.5 | |
| 3537 | 12.79554171 | down | 4.62E−09 | 0.00000202 | BE822929 | 665 | 49 | 82 | 59 | 4e−23 | cell adhesion |
| 3538 | 12.79534382 | down | 0.000000491 | 0.000022 | CD417435 | 530 | 28 | 62 | 45 | 2e−05 | Glutamate 1-semialdehyde |
| | | | | | | | | | | | aminotransferase |
| 3539 | 12.79516626 | down | 5.45E−09 | 0.00000216 | BI973233 | 502 | 62 | 99 | 62 | 6e−34 | defense response |
| 3540 | 12.76473816 | down | 0.00000212 | 0.000053 | BE823195 | 667 | 115 | 136 | 84 | 6e−58 | nucleosome assembly |
| 3541 | 12.74379192 | down | 4.03E−08 | 0.0000053 | AW348438 | 756 | 120 | 174 | 68 | 3e−68 | transport |
| 3542 | 12.70220762 | down | 2.08E−08 | 0.00000385 | AW349314 | 760 | 93 | 161 | 57 | 1e−54 | |
| 3543 | 12.67446691 | down | 0.000185314 | 0.001065975 | BE023016 | 422 | 62 | 137 | 45 | 7e−27 | electron transport |
| 3544 | 12.67153496 | down | 0.00000354 | 0.0000729 | BQ081719 | 542 | 41 | 72 | 56 | 1e−22 | basic amino acid transport |
| 3545 | 12.59810627 | down | 6.48E−09 | 0.00000225 | AW309284 | 686 | 73 | 127 | 57 | 4e−38 | |
| 3546 | 12.57659526 | down | 1.29E−09 | 0.0000011 | AW348715 | 729 | 153 | 206 | 74 | 3e−80 | protein amino acid phosphorylation |
| 3547 | 12.55784156 | down | 4.56E−09 | 0.00000202 | BI968966 | 519 | 37 | 104 | 35 | 1e−15 | |
| 3548 | 12.49403642 | down | 9.56E−09 | 0.00000285 | CF806709 | 711 | 138 | 204 | 67 | 1e−73 | |
| 3549 | 12.45770814 | down | 0.00000012 | 0.00000969 | CD397881 | 621 | 104 | 135 | 77 | 1e−50 | nucleosome assembly |
| 3550 | 12.29088753 | down | 7.81E−08 | 0.00000757 | CD413702 | 601 | 80 | 86 | 83 | 1e−43 | microtubule-based process |
| 3551 | 12.2735814 | down | 0.00000638 | 0.000105523 | CD401279 | 658 | 82 | 147 | 55 | 1e−44 | |
| 3552 | 12.25759785 | down | 0.0000011 | 0.0000351 | CF806342 | 671 | 100 | 221 | 45 | 1e−56 | carbohydrate metabolism |
| 3553 | 12.22193365 | down | 0.00000154 | 0.0000429 | AI460621 | 648 | 136 | 200 | 68 | 1e−72 | proline biosynthesis |
| 3554 | 12.22023059 | down | 0.000000339 | 0.0000175 | CF807325 | 604 | 104 | 135 | 77 | 1e−54 | |
| 3555 | 12.21598756 | down | 0.000000305 | 0.0000164 | BQ627617 | 400 | 37 | 73 | 50 | 2e−15 | carbohydrate metabolism |
| 3556 | 12.2051364 | down | 5.61E−10 | 0.000000753 | CK605568 | 524 | 14 | 47 | 29 | 6.3 | |
| 3557 | 12.1550684 | down | 2.03E−08 | 0.00000381 | CF807078 | 519 | 93 | 137 | 67 | 5e−53 | peroxidase, response to dessication |
| 3558 | 12.15146596 | down | 0.00000351 | 0.0000726 | CD396418 | 679 | 35 | 56 | 62 | 3e−15 | |
| 3559 | 12.14875865 | down | 9.27E−08 | 0.0000083 | BM731979 | 521 | 61 | 155 | 39 | 7e−33 | cellulose biosynthesis |
| 3560 | 12.10700871 | down | 0.000000116 | 0.00000943 | AI794712 | 392 | 26 | 38 | 68 | 1e−09 | electron transport |
| 3561 | 12.10536505 | down | 2.43E−08 | 0.00000413 | AB008540.1 | | | | | | phosphoenolpyruvate carboxylase |
| 3562 | 12.08895794 | down | 3.98E−09 | 0.00000192 | CA852377 | 386 | 12 | 28 | 42 | 0.85 | |
| 3563 | 12.03216128 | down | 7.62E−08 | 0.00000746 | CD415329 | 641 | 11 | 19 | 57 | 1.4 | |
| 3564 | 11.99006981 | down | 0.0000126 | 0.000164279 | CA820526 | 509 | 18 | 65 | 27 | 2.0 | |
| 3565 | 11.9319291 | down | 0.0000935 | 0.000657032 | CD406772 | 749 | 77 | 150 | 51 | 8e−36 | metabolism |
| 3566 | 11.92280055 | down | 0.000000015 | 0.00000353 | AW432825 | 357 | 36 | 86 | 41 | 6e−12 | proteolysis and peptidolysis |
| 3567 | 11.88750352 | down | 0.000000132 | 0.0000102 | BI701903 | 329 | 12 | 37 | 32 | 0.86 | |
| 3568 | 11.87339188 | down | 0.000000282 | 0.0000156 | BM271361 | 416 | 14 | 14 | 100 | 0.014 | cell-cell signaling |
| 3569 | 17.67184963 | down | 4.86E−08 | 0.00000587 | BM269985 | 281 | 10 | 31 | 32 | 1.1 | |
| 3570 | 17.64992451 | down | 0.000000588 | 0.0000245 | BI967168 | 738 | 70 | 181 | 38 | 6e−31 | |
| 3571 | 17.47542353 | down | 0.000000147 | 0.0000108 | CD413702 | 601 | 80 | 86 | 93 | 1e−43 | microtubule-based process |
| 3572 | 17.31634787 | down | 3.63E−09 | 0.00000187 | AW350318 | 824 | 101 | 166 | 60 | 7e−55 | response to UV |
| 3573 | 17.23641092 | down | 0.000264535 | 0.001382933 | CK605701 | 657 | 59 | 174 | 33 | 2e−24 | |
| 3574 | 17.0769295 | down | 9.96E−09 | 0.00000293 | CD410832 | 436 | 20 | 56 | 35 | 1.5 | amino acid transport |
| 3575 | 16.95628364 | down | 0.000000266 | 0.0000149 | BU764871 | 392 | 19 | 37 | 51 | 0.004 | |
| 3576 | 16.94120691 | down | 9.45E−08 | 0.00000839 | CF921756 | 676 | 107 | 185 | 57 | 7e−56 | response to oxidative stress |
| 3577 | 16.88286985 | down | 0.000000073 | 0.00000738 | BU082252 | 377 | 11 | 28 | 39 | 1.3 | transport |
| 3578 | 16.86953174 | down | 6.22E−09 | 0.00000221 | BI967493 | 420 | 17 | 58 | 29 | 1.3 | cell adhesion |
| 3579 | 16.8587726 | down | 6.63E−09 | 0.00000225 | BU765628 | 591 | 66 | 121 | 54 | 2e−29 | electron transport |
| 3580 | 16.83231692 | down | 1.64E−08 | 0.00000353 | BQ628412 | 355 | 8 | 14 | 57 | 9.6 | |
| 3581 | 16.80019242 | down | 3.43E−08 | 0.00000493 | CF808642 | 711 | 160 | 229 | 69 | 8e−93 | peroxidase precursor, response to |
| | | | | | | | | | | | dessication |
| 3582 | 16.67012595 | down | 0.000670352 | 0.002721197 | CD390794 | 618 | 43 | 135 | 31 | 7e−10 | |
| 3583 | 16.60724987 | down | 3.42E−09 | 0.00000183 | BI970708 | 766 | 138 | 178 | 77 | 1e−82 | |
| 3584 | 16.58295005 | down | 0.00052576 | 0.002281753 | AW349743 | 561 | 44 | 84 | 52 | 9e−24 | |
| 3585 | 16.56831071 | down | 0.000683794 | 0.002757609 | BQ628396 | 316 | 8 | 17 | 47 | 4.2 | |
| 3586 | 16.50053024 | down | 6.99E−08 | 0.00000729 | BE609637 | 256 | 11 | 40 | 27 | 7.2 | |
| 3587 | 16.49788157 | down | 1.22E−08 | 0.00000309 | CF807078 | 519 | 93 | 137 | 67 | 5e−53 | Peroxidase (PC7), response to |
| | | | | | | | | | | | dessication |
| 3588 | 16.44541055 | down | 0.000000242 | 0.0000143 | CA802180 | 482 | 28 | 111 | 25 | 2e−05 | |
| 3589 | 16.43396607 | down | 3.94E−09 | 0.00000192 | AW309606 | 419 | 41 | 53 | 77 | 1e−17 | response to oxidative stress |
| 3590 | 16.40885259 | down | 2.46E−08 | 0.00000414 | BQ612647 | 520 | 72 | 117 | 61 | 6e−38 | lipid transport |
| 3591 | 16.40387002 | down | 0.000000141 | 0.0000106 | BI969027 | 556 | 67 | 160 | 41 | 3e−34 | |
| 3592 | 16.31664163 | down | 0.000136231 | 0.000857459 | BE020100 | 266 | 15 | 62 | 24 | 7.2 | regulation of transcription |
| 3593 | 16.22858256 | down | 0.000000198 | 0.0000128 | AW348242 | 721 | 98 | 148 | 66 | 1e−49 | carbohydrate metabolism |
| 3594 | 16.17805681 | down | 1.64E−10 | 0.000000589 | CF807494 | 728 | 86 | 130 | 66 | 3e−46 | fatty acid alpha-oxidation |
| 3595 | 16.13289131 | down | 6.25E−10 | 0.00000078 | AW310338 | 623 | 85 | 136 | 62 | 3e−43 | transport |
| 3596 | 15.92878198 | down | 0.000000244 | 0.0000144 | CK605560 | 600 | 31 | 125 | 24 | 0.95 | |
| 3597 | 15.91921528 | down | 0.0000233 | 0.000249858 | BM523459 | 589 | 44 | 128 | 34 | 4e−17 | |
| 3598 | 15.89336088 | down | 0.000160387 | 0.000963928 | CD395324 | 473 | 12 | 40 | 30 | 5.1 | |
| 3599 | 15.79411659 | down | 0.000000018 | 0.00000368 | BM522826 | 573 | 60 | 191 | 31 | 2e−23 | electron transport |
| 3600 | 15.68601727 | down | 1.51E−08 | 0.00000353 | AI966178 | 250 | 16 | 50 | 32 | 4.1 | peroxidase |
| 3601 | 15.66849738 | down | 2.4E−09 | 0.0000015 | AW278173 | 498 | 16 | 31 | 51 | 0.40 | |
| 3602 | 15.55823485 | down | 6.44E−09 | 0.00000225 | CF808620 | 710 | 186 | 216 | 86 | e−101 | glycolysis |
| 3603 | 15.54603645 | down | 7.04E−09 | 0.00000234 | CD414773 | 677 | 115 | 167 | 68 | 2e−65 | carbohydrate metabolism |
| 3604 | 15.54032422 | down | 5.93E−08 | 0.00000668 | BQ297915 | 583 | 95 | 110 | 86 | 7e−51 | galactose metabolism |
| 3605 | 15.51588086 | down | 3.62E−08 | 0.00000504 | BI970553 | 778 | 141 | 210 | 67 | 5e−84 | |
| 3606 | 15.35791277 | down | 0.00031277 | 0.00156464 | AW100848 | 338 | 11 | 32 | 34 | 2.5 | protein amino acid phosphorylation |
| 3607 | 15.33706793 | down | 0.0000224 | 0.000243053 | BG237174 | | | | | | |
| 3608 | 15.21292507 | down | 7.77E−08 | 0.00000757 | BE658675 | 317 | 31 | 48 | 64 | 2e−13 | ciliary/flagellar motility |
| 3609 | 15.18563503 | down | 3.77E−08 | 0.00000512 | CF806287 | 668 | 196 | 219 | 89 | e−114 | |
| 3610 | 15.17215009 | down | 2.79E−08 | 0.00000439 | BU762481 | 461 | 16 | 48 | 33 | 4.9 | transport |
| 3611 | 15.11866973 | down | 9.57E−08 | 0.00000844 | BE820934 | 765 | 94 | 123 | 76 | 4e−50 | acetyl-CoA biosynthesis |
| 3612 | 15.10105817 | down | 1.64E−09 | 0.00000129 | BI945137 | 416 | 19 | 38 | 50 | 0.024 | |
| 3613 | 15.08319792 | down | 5.09E−09 | 0.00000215 | BI967228 | 726 | 123 | 162 | 75 | 8e−67 | fatty acid alpha-oxidation |
| 3614 | 15.08271959 | down | 0.0000111 | 0.000151052 | CF806812 | 560 | 46 | 78 | 58 | 1e−22 | carbohydrate metabolism |
| 3615 | 15.02698373 | down | 0.0000698 | 0.000534007 | AF007211.1 | | | | | | peroxidase precursor |
| 3616 | 14.80706881 | down | 4.4E−09 | 0.00000202 | BE021801 | 601 | 19 | 71 | 26 | 1.2 | |
| 3617 | 14.71281044 | down | 0.00000476 | 0.0000878 | CD415317 | 468 | 27 | 71 | 38 | 2e−07 | |
| 3618 | 14.63849659 | down | 2.18E−08 | 0.00000392 | AW349280 | 761 | 65 | 126 | 51 | 5e−37 | cell wall biosynthesis |
| 3619 | 14.63091588 | down | 0.000128143 | 0.000822518 | CK605699 | 612 | 48 | 160 | 30 | 6e−06 | |
| 3620 | 14.59100831 | down | 1.58E−08 | 0.00000353 | CD402150 | 600 | 35 | 73 | 47 | 4e−15 | |
| 3621 | 14.54829547 | down | 9.2E−09 | 0.00000279 | BG237376 | 430 | 109 | 143 | 76 | 2e−61 | |
| 3622 | 14.54357105 | down | 3.69E−08 | 0.00000508 | BG156396 | 662 | 72 | 153 | 47 | 2e−30 | |
| 3623 | 14.49439636 | down | 4.27E−08 | 0.00000545 | BQ627520 | 341 | 10 | 23 | 43 | 1.1 | |
| 3624 | 14.49211798 | down | 0.00000355 | 0.000073 | BQ612490 | 261 | 13 | 44 | 29 | 5.5 | transport |
| 3625 | 14.43217965 | down | 0.0000137 | 0.000174178 | BQ611430 | 420 | 11 | 38 | 28 | 3.9 | |
| 3626 | 14.17122075 | down | 1.99E−10 | 0.000000589 | CD399255 | | | | | | |
| 3627 | 14.16085804 | down | 2.81E−08 | 0.00000439 | CD397515 | 682 | 60 | 97 | 61 | 3e−30 | |
| 3628 | 14.13668576 | down | 7.54E−08 | 0.00000744 | CD397201 | 446 | 27 | 43 | 62 | 6e−10 | |
| 3629 | 14.07628809 | down | 0.00000786 | 0.000119344 | CA802722 | 265 | 8 | 16 | 50 | 9.4 | acid phosphatase |
| 3630 | 13.98462856 | down | 0.00000186 | 0.0000484 | BE659234 | 392 | 35 | 35 | 100 | 1e−15 | microtubule-based movement |
| 3631 | 13.9821629 | down | 7.52E−08 | 0.00000744 | BI701093 | 426 | 64 | 86 | 74 | 8e−36 | cation transport |
| 3632 | 13.95794212 | down | 1.19E−08 | 0.00000307 | BI971286 | 559 | 82 | 148 | 55 | 1e−39 | response to dessication |
| 3633 | 13.88019439 | down | 0.0000399 | 0.000361044 | BI945123 | | | | | | |
| 3634 | 13.83202255 | down | 6.11E−09 | 0.00000221 | AI856053 | 546 | 11 | 21 | 52 | 2.3 | |
| 3635 | 13.8202006 | down | 2.84E−08 | 0.0000044 | BI969778 | 677 | 104 | 177 | 58 | 5e−60 | defense response |
| 3636 | 13.77828998 | down | 0.00000149 | 0.0000418 | CD414058 | 608 | 58 | 74 | 78 | 2e−29 | proteolysis and peptidolysis |
| 3637 | 13.75252454 | down | 0.000000413 | 0.0000198 | CA936529 | 425 | 22 | 42 | 52 | 2e−07 | |
| 3638 | 13.73964594 | down | 0.00000118 | 0.0000364 | AW203386 | 546 | 80 | 167 | 47 | 2e−36 | metabolism |
| 3639 | 13.7255303 | down | 0.000864601 | 0.003280653 | AW831290 | 433 | 69 | 142 | 48 | 4e−34 | electron transport |
| 3640 | 13.70790611 | down | 0.000000262 | 0.0000149 | CD402416 | 580 | 44 | 78 | 56 | 1e−18 | proteolysis and peptidolysis |
| 3641 | 9.372522048 | down | 0.000000517 | 0.0000226 | CF807156 | 653 | 92 | 149 | 61 | 4e−52 | PR1a precursor |
| 3642 | 9.364067028 | down | 0.00000154 | 0.0000429 | BQ612457 | 299 | 9 | 28 | 32 | 4.2 | apoptosis |
| 3643 | 9.287630827 | down | 0.000000539 | 0.0000234 | CA799123 | 741 | 133 | 230 | 57 | 8e−75 | proteolysis and peptidolysis |
| 3644 | 9.28184954 | down | 0.000337721 | 0.001649744 | BQ610818 | 230 | 10 | 33 | 30 | 9.3 | |
| 3645 | 9.232955313 | down | 0.00000533 | 0.0000945 | BI273670 | 522 | 69 | 122 | 56 | 5e−34 | response to oxidative stress |
| 3646 | 9.226506446 | down | 0.000000114 | 0.00000929 | CD395530 | 688 | 91 | 122 | 74 | 5e−44 | nucleosome assembly |
| 3647 | 9.203033448 | down | 0.000000205 | 0.0000131 | BM567981 | 582 | 71 | 145 | 48 | 5e−29 | regulation of transcription |
| 3648 | 9.199937214 | down | 3.63E−08 | 0.00000504 | CD390967 | 689 | 101 | 153 | 66 | 1e−61 | carbohydrate metabolism |
| 3649 | 9.196303442 | down | 0.00000105 | 0.0000342 | BI943979 | 329 | 15 | 49 | 30 | 1.5 | translational termination |
| 3650 | 9.193685661 | down | 0.000015 | 0.000185405 | AF007211.1 | | | | | | peroxidase precursor |
| 3651 | 9.18581629 | down | 0.0000063 | 0.000104931 | BI784578 | 412 | 67 | 104 | 64 | 1e−31 | polygalacturonase PG1 /// |
| | | | | | | | | | | | polygalacturonase PG2, carbohydrate |
| | | | | | | | | | | | metabolism |
| 3652 | 9.15621988 | down | 0.0000105 | 0.000144642 | BU926461 | 614 | 121 | 147 | 82 | 2e−72 | carbohydrate metabolism |
| 3653 | 9.150151594 | down | 0.00000115 | 0.0000361 | BF595464 | 443 | 32 | 66 | 48 | 1e−08 | transport |
| 3654 | 9.138875724 | down | 0.00000666 | 0.000107999 | BQ611686 | 330 | 22 | 41 | 53 | 6e−07 | |
| 3655 | 9.127279345 | down | 0.00000653 | 0.000106503 | BI316058 | 416 | 66 | 148 | 44 | 5e−27 | carbon utilization by fixation of carbon |
| | | | | | | | | | | | dioxide |
| 3656 | 9.086264131 | down | 0.000000421 | 0.0000201 | BM522529 | 332 | 30 | 55 | 54 | 1e−12 | |
| 3657 | 9.081200506 | down | 0.00000007 | 0.00000729 | CA819378 | 413 | 36 | 67 | 53 | 2e−17 | chemotaxis |
| 3658 | 9.059209101 | down | 0.00000053 | 0.0000231 | BI970072 | 786 | 93 | 190 | 48 | 8e−50 | response to dessication |
| 3659 | 9.036245301 | down | 3.21E−08 | 0.00000473 | CF805992 | 692 | 25 | 88 | 28 | 0.038 | |
| 3660 | 9.03469851 | down | 0.000280452 | 0.001444661 | BG511562 | 351 | 15 | 56 | 26 | 4.2 | |
| 3661 | 9.021942271 | down | 0.00000015 | 0.000011 | AW309460 | 512 | 60 | 92 | 65 | 1e−36 | |
| 3662 | 9.005590008 | down | 0.000000783 | 0.0000291 | AW309561 | 652 | 94 | 149 | 63 | 8e−49 | |
| 3663 | 8.902068123 | down | 4.99E−09 | 0.00000213 | BI702248 | 441 | 76 | 115 | 66 | 2e−36 | |
| 3664 | 8.891757464 | down | 0.000000011 | 0.00000298 | BQ081095 | 542 | 74 | 167 | 44 | 5e−36 | metabolism |
| 3665 | 8.859396166 | down | 0.000000258 | 0.0000148 | BU965333 | 476 | 69 | 87 | 79 | 3e−36 | protein amino acid phosphorylation |
| 3666 | 8.802433708 | down | 0.00000386 | 0.0000771 | CD402690 | 569 | 55 | 77 | 71 | 2e−33 | |
| 3667 | 8.796331406 | down | 1.97E−08 | 0.00000378 | AW349894 | 787 | 65 | 102 | 63 | 2e−31 | ADR11 mRNA |
| 3668 | 8.790497381 | down | 0.00000783 | 0.000119185 | BM567831 | 577 | 69 | 93 | 74 | 1e−36 | proline biosynthesis |
| 3669 | 8.774213891 | down | 2.67E−09 | 0.00000162 | BI973382 | 561 | 94 | 198 | 47 | 2e−50 | |
| 3670 | 8.764388424 | down | 0.000000489 | 0.000022 | CD415063 | 608 | 21 | 37 | 56 | 2e−06 | |
| 3671 | 4.263870423 | down | 0.000218414 | 0.00120302 | AF184277.2 | | | | | | homeodomain-leucine zipper protein 56 |
| 3672 | 4.263200782 | down | 0.000000143 | 0.0000107 | BQ610588 | 444 | 45 | 50 | 90 | 9e−19 | response to osmotic stress |
| 3673 | 4.262617384 | down | 0.000000605 | 0.0000249 | CF809068 | 600 | 30 | 47 | 63 | 3e−14 | |
| 3674 | 4.262607132 | down | 0.000000225 | 0.0000139 | BQ080496 | 577 | 109 | 163 | 66 | 9e−59 | response to oxidative stress |
| 3675 | 4.25953391 | down | 0.00000796 | 0.000120296 | CA802819 | 792 | 139 | 172 | 80 | 2e−77 | ‘de novo’ pyrimidine base biosynthesis |
| 3676 | 4.258905643 | down | 0.00000433 | 0.000083 | AJ563382.1 | | | | | | ornithine decarboxylase |
| 3677 | 4.257783443 | down | 0.00000435 | 0.0000833 | BI425574 | 520 | 55 | 94 | 58 | 4e−28 | |
| 3678 | 4.257601166 | down | 0.0000132 | 0.000170273 | BM523279 | | | | | | |
| 3679 | 4.257294933 | down | 0.0000127 | 0.000165054 | AW101408 | | | | | | |
| 3680 | 4.256734027 | down | 0.000000263 | 0.0000149 | CD402264 | 589 | 65 | 105 | 61 | 1e−31 | |
| 3681 | 4.255648808 | down | 0.0000501 | 0.00042312 | AW830233 | 488 | 71 | 161 | 44 | 4e−37 | electron transport |
| 3682 | 4.248226165 | down | 7.57E−08 | 0.00000744 | BE821176 | 374 | 28 | 68 | 41 | 2e−07 | |
| 3683 | 4.245070101 | down | 0.0000103 | 0.000143074 | BM086044 | 570 | 144 | 189 | 76 | 7e−78 | |
| 3684 | 4.244134316 | down | 0.000000668 | 0.0000267 | AW348133 | 445 | 34 | 56 | 60 | 8e−10 | |
| 3685 | 4.243331863 | down | 0.00000313 | 0.0000678 | CF922946 | 747 | 61 | 90 | 67 | 7e−34 | electron transport |
| 3686 | 4.242609692 | down | 0.00000273 | 0.0000619 | BG237903 | 321 | 14 | 51 | 27 | 0.64 | transport |
| 3687 | 4.241142221 | down | 0.00000174 | 0.0000463 | AJ563365.1 | | | | | | monosaccharide transporter |
| 3688 | 4.240800412 | down | 0.000000432 | 0.0000205 | AI938431 | 333 | 17 | 29 | 58 | 0.045 | protein amino acid phosphorylation |
| 3689 | 4.239510493 | down | 0.00000354 | 0.0000729 | AW394548 | 212 | 20 | 41 | 48 | 7e−05 | DNA recombination |
| 3690 | 4.236529081 | down | 0.000206468 | 0.001153607 | BM309694 | 407 | 14 | 31 | 45 | 3.7 | |
| 3691 | 4.234162181 | down | 0.00000716 | 0.00011286 | CD390845 | 652 | 63 | 102 | 61 | 3e−33 | |
| 3692 | 4.233825963 | down | 0.00000603 | 0.000101686 | BU548398 | 501 | 12 | 32 | 37 | 1.2 | regulation of transcription\, DNA- |
| | | | | | | | | | | | dependent |
| 3693 | 4.230914572 | down | 0.000000777 | 0.0000291 | CA785238 | 742 | 205 | 230 | 89 | e−114 | |
| 3694 | 4.229599552 | down | 0.0000231 | 0.000248833 | BM093843 | 223 | 10 | 43 | 23 | 0.85 | nucleosome assembly |
| 3695 | 4.228878402 | down | 0.000000226 | 0.0000139 | BQ081639 | 540 | 117 | 186 | 62 | 1e−54 | |
| 3696 | 4.228081969 | down | 0.0000153 | 0.000188532 | CA935964 | 160 | 9 | 19 | 47 | 1.1 | |
| 3697 | 4.226516663 | down | 0.000000782 | 0.0000291 | BQ080218 | 547 | 66 | 135 | 48 | 8e−33 | electron transport |
| 3698 | 13.67844564 | down | 0.000000217 | 0.0000137 | CF809278 | 536 | 87 | 182 | 47 | 3e−42 | lipoxygenase /// lipoxygenase |
| 3699 | 13.65373805 | down | 0.0000663 | 0.000515257 | BI943300 | | | | | | |
| 3700 | 13.63808319 | down | 0.0000108 | 0.000147495 | AW395005 | 617 | 76 | 155 | 49 | 2e−41 | Cytochrome P450 |
| | | | | | | | | | | | monooxygenaseCYP93D1 (CYP93E1), |
| | | | | | | | | | | | electron transport |
| 3701 | 13.63368185 | down | 0.000000205 | 0.0000131 | BE330075 | 497 | 12 | 32 | 37 | 0.52 | metabolism |
| 3702 | 13.61728466 | down | 1.14E−08 | 0.00000304 | U51194.1 | | | | | | peroxidase |
| 3703 | 13.61014738 | down | 5.43E−08 | 0.00000639 | CD412339 | 707 | 133 | 136 | 97 | 6e−69 | nucleosome assembly |
| 3704 | 13.60735834 | down | 7.05E−08 | 0.00000729 | CA818952 | 372 | 17 | 62 | 27 | 1.00 | |
| 3705 | 13.57986351 | down | 0.0000369 | 0.000343074 | AI441778 | 416 | 72 | 135 | 53 | 3e−41 | |
| 3706 | 13.50342146 | down | 0.000000304 | 0.0000164 | BE820448 | 457 | 21 | 64 | 32 | 0.33 | |
| 3707 | 13.45292225 | down | 0.000000129 | 0.0000101 | BQ611806 | 169 | 10 | 15 | 66 | 4.2 | electron transport |
| 3708 | 13.39575565 | down | 2.1E−09 | 0.00000146 | AF047718.1 | | | | | | NRT2 protein |
| 3709 | 13.38609556 | down | 3.47E−08 | 0.00000495 | BG881473 | 395 | 48 | 76 | 63 | 2e−22 | |
| 3710 | 13.36340038 | down | 9.13E−08 | 0.00000821 | BG789983 | 516 | 49 | 169 | 28 | 8e−16 | |
| 3711 | 13.35009562 | down | 2.23E−09 | 0.00000146 | BE658101 | 752 | 100 | 154 | 64 | 1e−59 | |
| 3712 | 13.29162761 | down | 1.07E−08 | 0.00000293 | BM523729 | 426 | 20 | 34 | 58 | 6e−06 | |
| 3713 | 13.18709494 | down | 3.76E−08 | 0.00000512 | AW132771 | 643 | 57 | 171 | 33 | 2e−21 | G-protein coupled receptor protein |
| | | | | | | | | | | | signaling pathway |
| 3714 | 13.17206559 | down | 3.49E−08 | 0.00000495 | BF598755 | 575 | 21 | 27 | 77 | 8e−08 | auxin down regulated |
| 3715 | 13.1707127 | down | 0.00000274 | 0.000062 | CD391960 | 660 | 36 | 47 | 76 | 5e−17 | |
| 3716 | 11.85801632 | down | 0.000000042 | 0.00000537 | BE657645 | 710 | 43 | 138 | 31 | 1e−12 | peptidoglycan metabolism |
| 3717 | 11.85653726 | down | 9.11E−08 | 0.00000821 | BI316021 | 504 | 150 | 168 | 89 | 2e−91 | |
| 3718 | 11.83154186 | down | 0.000000161 | 0.0000114 | BQ627558 | 178 | 9 | 21 | 42 | 7.2 | |
| 3719 | 11.80157317 | down | 4.95E−10 | 0.000000745 | BQ298229 | | | | | | |
| 3720 | 11.73377057 | down | 0.00000862 | 0.000126391 | CD393884 | 415 | 19 | 42 | 45 | 0.024 | |
| 3721 | 11.70697509 | down | 4.34E−09 | 0.00000202 | AI901267 | 515 | 25 | 52 | 48 | 4e−08 | |
| 3722 | 11.69022137 | down | 0.000000101 | 0.00000866 | CD393884 | 415 | 19 | 42 | 45 | 0.024 | |
| 3723 | 11.67838872 | down | 0.000000349 | 0.0000178 | CF807802 | 739 | 153 | 238 | 64 | 4e−87 | electron transport |
| 3724 | 11.64596422 | down | 0.000000752 | 0.0000286 | BI967474 | 644 | 78 | 110 | 70 | 2e−43 | chitin catabolism |
| 3725 | 11.63542475 | down | 0.0000835 | 0.000605612 | BM526581 | 541 | 49 | 178 | 27 | 3e−15 | response to wounding |
| 3726 | 11.61272652 | down | 0.00000401 | 0.0000789 | AW310433 | 668 | 92 | 133 | 69 | 3e−49 | G beta-like protein |
| 3727 | 11.61060546 | down | 6.19E−08 | 0.00000682 | CK606333 | 700 | 124 | 201 | 61 | 1e−66 | |
| 3728 | 11.51350175 | down | 0.000000366 | 0.0000184 | CD400520 | 609 | 135 | 136 | 99 | 9e−71 | nucleosome assembly |
| 3729 | 11.45670556 | down | 0.000000235 | 0.0000141 | BE658675 | 317 | 31 | 48 | 64 | 2e−13 | ciliary/flagellar motility |
| 3730 | 11.26185644 | down | 0.0000236 | 0.000252543 | M20038.1 | | | | | | 31 kDa protein |
| 3731 | 11.24806278 | down | 0.000000193 | 0.0000127 | BE440266 | 425 | 31 | 43 | 72 | 2e−12 | |
| 3732 | 11.20727499 | down | 3.18E−08 | 0.00000472 | BM139729 | 281 | 19 | 29 | 65 | 1e−07 | response to dessication |
| 3733 | 11.1937509 | down | 5.41E−08 | 0.00000638 | CD406086 | 741 | 128 | 135 | 94 | 2e−72 | microtubule-based process |
| 3734 | 11.16563982 | down | 0.000000258 | 0.0000148 | CF808723 | 578 | 66 | 192 | 34 | 6e−24 | |
| 3735 | 7.997733134 | down | 0.00000226 | 0.00006554 | BM271358 | 310 | 9 | 20 | 45 | 2.5 | metabolism |
| 3736 | 7.988093514 | down | 0.000000907 | 0.0000318 | BQ611014 | 445 | 22 | 54 | 40 | 3e−05 | metabolism |
| 3737 | 7.981797484 | down | 7.83E−08 | 0.00000758 | BE059139 | 435 | 17 | 63 | 26 | 7.3 | regulation of transcription\, DNA- |
| | | | | | | | | | | | dependent |
| 3738 | 7.962998989 | down | 0.000000467 | 0.0000214 | CD402282 | 679 | 107 | 130 | 82 | 3e−53 | nucleosome assembly |
| 3739 | 7.949576303 | down | 0.000000274 | 0.0000152 | BI971102 | 771 | 120 | 166 | 72 | 1e−68 | |
| 3740 | 7.933071126 | down | 0.00000413 | 0.0000803 | AW706349 | | | | | | |
| 3741 | 7.923636486 | down | 0.000000057 | 0.00000658 | BQ454274 | 439 | 13 | 36 | 36 | 0.67 | |
| 3742 | 7.922642501 | down | 0.000000152 | 0.000011 | BG653611 | 499 | 70 | 143 | 48 | 3e−32 | resistance protein MG13 |
| 3743 | 7.918536708 | down | 2.19E−08 | 0.00000392 | CF806997 | 509 | 94 | 146 | 64 | 2e−56 | |
| 3744 | 7.917509513 | down | 0.00000148 | 0.0000417 | AW350953 | 755 | 170 | 196 | 86 | 1e−96 | microtubule-based movement |
| 3745 | 7.906110907 | down | 7.25E−08 | 0.00000737 | U50075.1 | | | | | | Lipoxygenase L-5 (vlxB) |
| 3746 | 6.170654989 | down | 0.000234063 | 0.001265674 | BG511716 | 286 | 15 | 46 | 32 | 3.2 | regulation of transcription\, DNA- |
| | | | | | | | | | | | dependent |
| 3747 | 6.17020375 | down | 4.88E−08 | 0.00000588 | BI971744 | 437 | 13 | 28 | 46 | 1.5 | |
| 3748 | 6.169272099 | down | 5.51E−09 | 0.00000216 | CD418071 | 588 | 75 | 126 | 59 | 9e−38 | |
| 3749 | 6.165008855 | down | 0.000000367 | 0.0000184 | CK606095 | 880 | 145 | 232 | 62 | 2e−86 | lipid metabolism |
| 3750 | 6.163587726 | down | 0.00000524 | 0.0000934 | BE820335 | 519 | 63 | 92 | 68 | 2e−31 | |
| 3751 | 6.156555991 | down | 0.000129406 | 0.000827944 | AJ003245.1 | | | | | | NADPH:isoflavone reductase |
| 3752 | 6.14800051 | down | 0.000000184 | 0.0000123 | BG511038 | 347 | 19 | 58 | 32 | 0.84 | |
| 3753 | 6.14450638 | down | 0.000000182 | 0.0000122 | BM309390 | 596 | 161 | 203 | 79 | 6e−90 | |
| 3754 | 6.139341947 | down | 0.000000145 | 0.0000108 | CD403181 | 656 | 13 | 32 | 40 | 1.9 | |
| 3755 | 6.137123516 | down | 0.00000145 | 0.0000412 | BQ473559 | 558 | 110 | 158 | 69 | 1e−57 | |
| 3756 | 6.13356368 | down | 0.000000126 | 0.0000099 | BI945026 | 651 | 60 | 81 | 74 | 1e−29 | carbohydrate metabolism |
| 3757 | 6.131458623 | down | 0.00000444 | 0.0000843 | CF806162 | 622 | 130 | 151 | 86 | 6e−73 | microtubule-based process |
| 3758 | 6.13140418 | down | 0.000000152 | 0.000011 | AW570537 | 214 | 58 | 58 | 100 | 3e−26 | nucleosome assembly |
| 3759 | 6.122694778 | down | 0.00000101 | 0.0000338 | BQ453209 | 451 | 19 | 60 | 31 | 4.6 | hypothetical LOC547692 |
| 3760 | 6.120700737 | down | 0.00000403 | 0.000079 | BQ611867 | 380 | 35 | 64 | 54 | 1e−15 | response to oxidative stress |
| 3761 | 6.117906559 | down | 0.00000101 | 0.0000338 | BE823288 | 416 | 16 | 51 | 31 | 1.3 | |
| 3762 | 6.117328908 | down | 0.00000451 | 0.000085 | BQ610899 | 207 | 14 | 41 | 34 | 0.30 | aromatic amino acid family metabolism |
| 3763 | 6.111415249 | down | 0.00000126 | 0.0000378 | BI969835 | 368 | 18 | 39 | 46 | 2e−06 | amino acid transport |
| 3764 | 6.109452376 | down | 0.001186261 | 0.004112065 | U04526.1 | | | | | | lipoxygenase |
| 3765 | 6.108698233 | down | 0.0000514 | 0.000431267 | AW310453 | 543 | 77 | 102 | 75 | 7e−40 | metabolism |
| 3766 | 6.105550796 | down | 0.00000087 | 0.000031 | AW310249 | 642 | 42 | 78 | 53 | 4e−18 | response to oxidative stress |
| 3767 | 6.085661641 | down | 0.000000315 | 0.0000167 | CD398988 | 535 | 43 | 68 | 63 | 4e−15 | |
| 3768 | 6.073259989 | down | 1.21E−08 | 0.00000309 | BF070874 | 462 | 18 | 39 | 46 | 0.011 | |
| 3769 | 6.044357127 | down | 4.49E−08 | 0.00000564 | CF808030 | 519 | 88 | 150 | 58 | 6e−43 | |
| 3770 | 11.14724881 | down | 0.000000048 | 0.00000586 | AW394783 | 638 | 22 | 22 | 100 | 3e−07 | |
| 3771 | 11.13496502 | down | 0.00000516 | 0.0000928 | BE805933 | 569 | 62 | 200 | 31 | 3e−23 | |
| 3772 | 11.12213221 | down | 9.49E−08 | 0.0000084 | BG881473 | 395 | 48 | 76 | 63 | 2e−22 | |
| 3773 | 11.10566226 | down | 0.000000227 | 0.0000139 | CF807372 | 748 | 139 | 254 | 54 | 1e−67 | |
| 3774 | 11.10558175 | down | 0.000000171 | 0.0000117 | CF809039 | 706 | 94 | 180 | 52 | 3e−49 | |
| 3775 | 11.0855788 | down | 4.55E−09 | 0.00000202 | AW349894 | 787 | 65 | 102 | 63 | 2e−31 | auxin down regulated |
| 3776 | 11.0560156 | down | 2.01E−09 | 0.00000146 | BM523361 | 270 | 11 | 15 | 73 | 5.5 | protein amino acid phosphorylation |
| 3777 | 11.05499128 | down | 0.000000031 | 0.00000467 | BE021541 | 583 | 57 | 135 | 42 | 6e−26 | |
| 3778 | 11.04038702 | down | 0.000000014 | 0.00000337 | CF808704 | 584 | 116 | 155 | 74 | 1e−62 | |
| 3779 | 10.97417137 | down | 3.62E−08 | 0.00000504 | BI970976 | 791 | 99 | 204 | 48 | 5e−46 | response to oxidative stress |
| 3780 | 10.96360094 | down | 0.000268133 | 0.00139689 | CA851637 | 583 | 52 | 61 | 85 | 2e−24 | transport |
| 3781 | 10.96257941 | down | 0.00000199 | 0.0000509 | CA800320 | 626 | 95 | 121 | 78 | 5e−53 | |
| 3782 | 10.95548406 | down | 0.00000131 | 0.0000384 | BQ610991 | 445 | 12 | 38 | 31 | 3.4 | cell growth and/or maintenance |
| 3783 | 10.94438209 | down | 0.000000019 | 0.00000375 | BU549975 | 657 | 64 | 171 | 37 | 2e−26 | |
| 3784 | 10.91660004 | down | 0.00000123 | 0.0000372 | CF806670 | 712 | 94 | 187 | 50 | 8e−45 | electron transport |
| 3785 | 10.87736833 | down | 1.78E−08 | 0.00000368 | CF807507 | 457 | 36 | 46 | 78 | 3e−17 | fatty acid alpha-oxidation |
| 3786 | 10.87376662 | down | 7.05E−08 | 0.00000729 | BF069978 | 375 | 16 | 49 | 32 | 2.9 | |
| 3787 | 10.86086986 | down | 5.48E−09 | 0.00000216 | CK605562 | 713 | 32 | 68 | 47 | 1e−12 | metal ion transport |
| 3788 | 10.8578058 | down | 0.000000213 | 0.0000135 | BU764648 | 360 | 10 | 22 | 45 | 3.4 | regulation of transcription\, DNA- |
| | | | | | | | | | | | dependent |
| 3789 | 10.82326954 | down | 0.0000169 | 0.000200912 | CD397298 | 582 | 72 | 133 | 54 | 1e−35 | response to wounding |
| 3790 | 10.8211362 | down | 2.26E−08 | 0.000004 | BI967629 | 551 | 57 | 90 | 63 | 1e−29 | |
| 3791 | 10.8179123 | down | 0.00000349 | 0.0000723 | BE823205 | 384 | 27 | 46 | 58 | 4e−11 | |
| 3792 | 10.80487903 | down | 0.000000332 | 0.0000173 | CD394574 | 717 | 66 | 145 | 45 | 2e−26 | regulation of transcription |
| 3793 | 10.79132099 | down | 0.000000467 | 0.0000214 | BU548101 | 678 | 49 | 178 | 27 | 3e−17 | |
| 3794 | 10.78901421 | down | 8.35E−08 | 0.00000783 | CA802612 | 210 | 14 | 54 | 25 | 0.86 | cytolysis |
| 3795 | 10.77156992 | down | 4.23E−09 | 0.00000201 | BM178864 | 376 | 22 | 60 | 36 | 2e−05 | |
| 3796 | 10.7366914 | down | 4.91E−08 | 0.0000059 | BQ740550 | 293 | 8 | 12 | 66 | 5.6 | protein-nucleus import |
| 3797 | 10.72906028 | down | 4.58E−09 | 0.00000202 | AW349424 | 718 | 60 | 132 | 45 | 6e−25 | cell adhesion |
| 3798 | 10.72794256 | down | 1.15E−09 | 0.0000011 | AW310286 | 641 | 86 | 123 | 69 | 8e−49 | electron transport |
| 3799 | 10.72391861 | down | 1.04E−08 | 0.00000293 | BI967598 | 763 | 97 | 149 | 65 | 9e−59 | |
| 3800 | 10.70697735 | down | 0.000000157 | 0.0000112 | BM522465 | 293 | 9 | 20 | 45 | 1.9 | |
| 3801 | 10.65615606 | down | 6.64E−10 | 0.00000078 | BU762481 | 461 | 16 | 48 | 33 | 4.9 | transport |
| 3802 | 10.64192641 | down | 0.000000942 | 0.0000325 | CD393765 | 419 | 44 | 46 | 95 | 5e−19 | nucleosome assembly |
| 3803 | 10.6114833 | down | 0.00000405 | 0.0000792 | CK606530 | 552 | 17 | 47 | 36 | 0.82 | |
| 3804 | 10.59758478 | down | 7.1E−09 | 0.00000234 | BE658819 | 305 | 12 | 26 | 46 | 7.2 | regulation of transcription\, DNA- |
| | | | | | | | | | | | dependent |
| 3805 | 10.59650579 | down | 0.001275946 | 0.00433741 | CD414209 | 644 | 37 | 72 | 51 | 2e−15 | electron transport |
| 3806 | 10.57285495 | down | 0.000000686 | 0.0000271 | CD391589 | 635 | 63 | 124 | 50 | 1e−32 | |
| 3807 | 10.5623 | down | 0.00000207 | 0.0000521 | CD404597 | 548 | 40 | 45 | 88 | 6e−17 | nucleosome assembly |
| 3808 | 10.55462492 | down | 0.000001 | 0.0000337 | CK605927 | 442 | 12 | 38 | 31 | 3.4 | Hydroxyproline-rich glycoprotein (hrgp), |
| | | | | | | | | | | | cell growth and/or maintenance |
| 3809 | 10.5173163 | down | 0.000000112 | 0.0000092 | AW311147 | 490 | 30 | 44 | 68 | 9e−14 | endo-xyloglucan transferase, |
| | | | | | | | | | | | carbohydrate metabolism |
| 3810 | 10.51015242 | down | 0.000000576 | 0.0000243 | BQ273560 | 540 | 87 | 178 | 48 | 9e−48 | protein amino acid phosphorylation |
| 3811 | 10.49953458 | down | 9.46E−08 | 0.00000839 | CK606168 | 822 | 149 | 258 | 57 | 4e−77 | biological_process unknown |
| 3812 | 10.49343848 | down | 9.91E−08 | 0.00000857 | BM892178 | 437 | 113 | 144 | 78 | 3e−64 | tip growth |
| 3813 | 10.48171265 | down | 0.000115025 | 0.000759845 | BQ611770 | 381 | 13 | 41 | 31 | 2.3 | peroxidase precursor /// peroxidase |
| | | | | | | | | | | | precursor |
| 3814 | 10.47898642 | down | 0.000000069 | 0.00000729 | CF806307 | 698 | 181 | 199 | 90 | e−103 | microtubule-based process |
| 3815 | 10.42089838 | down | 0.00000231 | 0.000056 | CA782166 | 227 | 11 | 23 | 47 | 9.3 | |
| 3816 | 10.40138101 | down | 2.25E−09 | 0.00000146 | CF809344 | 543 | 94 | 180 | 52 | 6e−46 | cell adhesion |
| 3817 | 10.38010697 | down | 0.00000129 | 0.0000382 | AI442263 | 344 | 71 | 83 | 85 | 3e−40 | |
| 3818 | 10.3783039 | down | 0.00000131 | 0.0000384 | AW310734 | 490 | 45 | 86 | 52 | 6e−21 | potassium ion transport |
| 3819 | 10.37338023 | down | 5.18E−09 | 0.00000216 | CF807610 | 564 | 91 | 114 | 79 | 3e−48 | glycoprotein catabolism |
| 3820 | 10.33939662 | down | 0.000000001 | 0.00000099 | BE822092 | 651 | 64 | 146 | 43 | 2e−24 | |
| 3821 | 10.31055753 | down | 7.11E−08 | 0.0000073 | AW666186 | 329 | 75 | 107 | 70 | 9e−40 | response to oxidative stress |
| 3822 | 10.25669114 | down | 0.000000334 | 0.0000173 | BG882760 | 424 | 61 | 100 | 61 | 4e−32 | |
| 3823 | 10.12489816 | down | 0.000000314 | 0.0000167 | CD409881 | 568 | 69 | 100 | 69 | 1e−35 | jasmonic acid biosynthesis |
| 3824 | 10.11972273 | down | 1.28E−09 | 0.0000011 | CD414114 | 650 | 87 | 135 | 64 | 9e−51 | |
| 3825 | 10.0926294 | down | 0.001122342 | 0.003960948 | BE820032 | 387 | 18 | 52 | 34 | 0.85 | |
| 3826 | 10.07381701 | down | 0.000000018 | 0.00000368 | BE823251 | 564 | 42 | 72 | 58 | 8e−20 | response to gibberellic acid stimulus |
| 3827 | 10.07101268 | down | 0.00000148 | 0.0000417 | CF805988 | 697 | 147 | 230 | 63 | 1e−80 | lipoxygenase |
| 3828 | 10.05620699 | down | 0.0000107 | 0.000146657 | BE658665 | 652 | 69 | 153 | 45 | 3e−30 | metabolism |
| 3829 | 10.05078981 | down | 2.28E−08 | 0.00000401 | CF807824 | 568 | 104 | 171 | 60 | 7e−57 | |
| 3830 | 10.04685598 | down | 0.00000199 | 0.0000509 | CK605646 | 526 | 54 | 128 | 42 | 2e−27 | lipoxygenase |
| 3831 | 10.02611206 | down | 0.00000109 | 0.0000351 | CD405503 | 698 | 110 | 196 | 56 | 1e−57 | |
| 3832 | 9.977471103 | down | 6.03E−08 | 0.00000677 | BE821339 | 682 | 14 | 31 | 45 | 1.2 | nucleobase\, nucleoside\, nucleotide |
| | | | | | | | | | | | and nucleic acid transport |
| 3833 | 9.967345811 | down | 0.0000723 | 0.000546885 | AY095999.1 | | | | | | beta-amyrin synthase |
| 3834 | 9.948993488 | down | 0.000000265 | 0.0000149 | CD391589 | 635 | 63 | 124 | 50 | 1e−32 | |
| 3835 | 9.941838725 | down | 0.0000509 | 0.00042809 | BU080867 | 350 | 28 | 40 | 70 | 7e−14 | |
| 3836 | 9.902514314 | down | 8.14E−09 | 0.00000255 | AW351171 | 460 | 14 | 29 | 48 | 0.006 | |
| 3837 | 9.893428155 | down | 2.33E−08 | 0.00000406 | CF807507 | 457 | 36 | 46 | 78 | 3e−17 | fatty acid alpha-oxidation |
| 3838 | 9.834695417 | down | 0.00000193 | 0.0000497 | CD402282 | 679 | 107 | 130 | 82 | 3e−53 | nucleosome assembly |
| 3839 | 9.834333618 | down | 3.12E−09 | 0.00000175 | CF809206 | 242 | 11 | 30 | 36 | 4.1 | G-protein coupled receptor protein |
| | | | | | | | | | | | signaling pathway |
| 3840 | 9.829418982 | down | 0.000000434 | 0.0000205 | AF128266.1 | | | | | | polygalacturonase PG1 |
| 3841 | 9.777712126 | down | 0.000000967 | 0.000033 | BF596776 | 473 | 130 | 157 | 82 | 4e−76 | |
| 3842 | 9.777665114 | down | 1.94E−08 | 0.00000376 | CF807728 | 566 | 82 | 167 | 49 | 3e−42 | response to UV |
| 3843 | 9.767274762 | down | 0.000000121 | 0.00000972 | CD410957 | 617 | 31 | 52 | 59 | 2e−14 | transport |
| 3844 | 9.764262346 | down | 5.72E−08 | 0.00000658 | CF806252 | 616 | 117 | 171 | 68 | 4e−69 | carbohydrate metabolism |
| 3845 | 9.707452199 | down | 0.0000032 | 0.0000684 | BE806816 | 466 | 112 | 154 | 72 | 2e−62 | |
| 3846 | 9.706150757 | down | 7.79E−08 | 0.00000757 | BI969195 | 684 | 91 | 174 | 52 | 3e−48 | antibiotic biosynthesis |
| 3847 | 9.706000191 | down | 8.35E−08 | 0.00000783 | CA800460 | 469 | 115 | 155 | 74 | 2e−61 | |
| 3848 | 9.681376705 | down | 0.00000373 | 0.0000753 | BU550200 | 662 | 47 | 117 | 40 | 6e−12 | boron transport |
| 3849 | 9.663840148 | down | 0.00000196 | 0.0000503 | CA936063 | 420 | 26 | 53 | 49 | 5e−10 | metabolism |
| 3850 | 9.593870692 | down | 0.000000253 | 0.0000147 | BG155109 | 279 | 12 | 39 | 30 | 2.4 | |
| 3851 | 9.584943244 | down | 0.00000494 | 0.0000902 | AW349654 | 601 | 59 | 123 | 47 | 1e−26 | response to auxin stimulus |
| 3852 | 9.578185064 | down | 0.00000925 | 0.000132616 | BI969674 | 743 | 87 | 174 | 50 | 7e−37 | |
| 3853 | 9.577765841 | down | 0.000120059 | 0.000784825 | AW396660 | 347 | 8 | 13 | 61 | 1.9 | |
| 3854 | 9.541955817 | down | 0.002002801 | 0.005988344 | L22029.1 | | | | | | hydroxyproline-rich glycoprotein |
| 3855 | 9.524949353 | down | 0.0000023 | 0.0000557 | CA785167 | 479 | 54 | 61 | 88 | 3e−28 | |
| 3856 | 9.522470554 | down | 0.000000139 | 0.0000106 | CA936681 | 270 | 41 | 84 | 48 | 8e−18 | metabolism |
| 3857 | 9.503205731 | down | 1.59E−08 | 0.00000353 | AW348396 | 792 | 164 | 221 | 74 | e−102 | chitin catabolism |
| 3858 | 9.503064564 | down | 0.000000387 | 0.000019 | CF809163 | 569 | 142 | 157 | 90 | 7e−81 | microtubule-based process |
| 3859 | 9.498991004 | down | 0.000000226 | 0.0000139 | BM188193 | 561 | 140 | 186 | 75 | 5e−83 | fatty acid alpha-oxidation |
| 3860 | 9.472436112 | down | 0.000000334 | 0.0000173 | CF805865 | 664 | 198 | 219 | 90 | e−113 | microtubule-based process |
| 3861 | 9.470470944 | down | 1.18E−09 | 0.0000011 | BQ612778 | 367 | 18 | 57 | 31 | 2.1 | |
| 3862 | 9.439220029 | down | 0.00000173 | 0.0000461 | AW733764 | 206 | 10 | 19 | 52 | 3.3 | electron transport |
| 3863 | 9.433925802 | down | 0.00000901 | 0.000130413 | BI702052 | 517 | 57 | 97 | 58 | 5e−28 | |
| 3864 | 9.428002205 | down | 2.87E−08 | 0.00000441 | BI316791 | 383 | 79 | 129 | 61 | 4e−43 | |
| 3865 | 8.729809222 | down | 0.00000104 | 0.0000341 | CD412352 | 627 | 82 | 82 | 100 | 1e−40 | nucleosome assembly |
| 3866 | 8.690222391 | down | 0.000348136 | 0.001686615 | CA802684 | 428 | 19 | 58 | 32 | 0.009 | response to wounding |
| 3867 | 8.67772011 | down | 1.17E−08 | 0.00000305 | CF921609 | 602 | 20 | 48 | 41 | 4e−05 | |
| 3868 | 8.67237617 | down | 0.000000953 | 0.0000327 | CA851713 | 362 | 50 | 98 | 51 | 1e−23 | DNA repair |
| 3869 | 8.620946005 | down | 3.46E−09 | 0.00000183 | AW100573 | 332 | 13 | 34 | 38 | 1.5 | |
| 3870 | 8.62007845 | down | 0.000000792 | 0.0000293 | BG650202 | 280 | 15 | 58 | 25 | 7.1 | |
| 3871 | 8.612346107 | down | 0.000000103 | 0.00000878 | BI892807 | 356 | 14 | 37 | 37 | 1.5 | |
| 3872 | 8.583260454 | down | 0.0000591 | 0.000475752 | BI971059 | 541 | 24 | 64 | 37 | 0.005 | |
| 3873 | 8.571782269 | down | 6.24E−08 | 0.00000682 | BU548215 | 589 | 90 | 183 | 49 | 2e−41 | |
| 3874 | 8.54132991 | down | 0.000000104 | 0.00000883 | CF807728 | 566 | 82 | 167 | 49 | 3e−42 | response to UV |
| 3875 | 8.518151719 | down | 0.000000335 | 0.0000173 | BI498123 | 554 | 112 | 184 | 60 | 8e−57 | ammonium transport |
| 3876 | 8.489671508 | down | 2.89E−08 | 0.00000443 | CA936082 | 443 | 20 | 49 | 40 | 0.010 | |
| 3877 | 8.479584784 | down | 1.61E−08 | 0.00000353 | CD412979 | 362 | 22 | 37 | 59 | 9e−07 | |
| 3878 | 8.454812574 | down | 0.00000445 | 0.0000843 | BQ611088 | 576 | 24 | 75 | 32 | 0.18 | proteolysis and peptidolysis |
| 3879 | 8.441249997 | down | 9.17E−09 | 0.00000279 | AW309491 | 451 | 16 | 35 | 45 | 0.41 | electron transport |
| 3880 | 8.439581975 | down | 0.001938526 | 0.005853393 | BU080591 | 556 | 18 | 50 | 36 | 0.020 | |
| 3881 | 8.435842563 | down | 0.00000563 | 0.0000977 | BE058741 | 263 | 14 | 18 | 77 | 0.29 | |
| 3882 | 8.418341352 | down | 0.000117133 | 0.000770117 | AW598371 | 298 | 11 | 23 | 47 | 3.2 | chloride transport |
| 3883 | 8.399568628 | down | 6.62E−08 | 0.0000071 | CD406782 | 629 | 135 | 136 | 99 | 1e−70 | nucleosome assembly |
| 3884 | 8.398217856 | down | 0.000000166 | 0.0000116 | CD413164 | 678 | 45 | 102 | 44 | 2e−14 | electron transport |
| 3885 | 8.382319619 | down | 0.000000384 | 0.000019 | CA801508 | 699 | 167 | 226 | 73 | 3e−90 | acetyl-CoA metabolism |
| 3886 | 8.36742883 | down | 0.000000258 | 0.0000148 | BQ612822 | 431 | 12 | 13 | 92 | 0.17 | |
| 3887 | 8.355757913 | down | 1.75E−08 | 0.00000368 | BI426462 | 571 | 49 | 90 | 54 | 1e−22 | galactose metabolism |
| 3888 | 8.349626851 | down | 0.000000216 | 0.0000136 | BF069978 | 375 | 16 | 49 | 32 | 2.9 | |
| 3889 | 8.299546513 | down | 3.93E−08 | 0.00000052 | AI443411 | 683 | 58 | 123 | 47 | 2e−25 | |
| 3890 | 8.295030706 | down | 3.99E−08 | 0.00000527 | BM178538 | 393 | 16 | 46 | 34 | 0.14 | regulation of translational initiation |
| 3891 | 8.294052878 | down | 0.00000405 | 0.0000792 | AI794712 | 392 | 26 | 38 | 68 | 1e−09 | electron transport |
| 3892 | 8.236138424 | down | 0.00000109 | 0.0000351 | BI944192 | 598 | 14 | 38 | 36 | 3.6 | |
| 3893 | 8.235284735 | down | 0.000101884 | 0.000696778 | BU765595 | 592 | 36 | 76 | 47 | 6e−18 | |
| 3894 | 8.233527926 | down | 1.22E−08 | 0.00000309 | BG791179 | 231 | 13 | 14 | 92 | 0.034 | |
| 3895 | 8.215802115 | down | 2.21E−08 | 0.00000394 | CF808466 | | | | | | |
| 3896 | 8.21091994 | down | 6.37E−08 | 0.0000069 | CF806474 | | | | | | |
| 3897 | 8.205805318 | down | 0.00000137 | 0.0000399 | BG237519 | 511 | 94 | 155 | 60 | 6e−55 | |
| 3898 | 8.20282369 | down | 6.64E−09 | 0.00000225 | BQ612879 | 613 | 19 | 41 | 46 | 0.002 | |
| 3899 | 8.202091843 | down | 0.000747667 | 0.002942018 | BI972239 | 392 | 42 | 65 | 64 | 2e−19 | proteolysis and peptidolysis |
| 3900 | 8.17516145 | down | 0.000000622 | 0.0000253 | AW507869 | 681 | 121 | 223 | 54 | 2e−66 | |
| 3901 | 8.16490529 | down | 0.000373058 | 0.001775776 | BI945212 | 440 | 18 | 34 | 52 | 3e−06 | proteolysis and peptidolysis |
| 3902 | 8.160065041 | down | 0.000492529 | 0.002174413 | AW349263 | 627 | 69 | 107 | 64 | 2e−33 | |
| 3903 | 8.158860713 | down | 0.00000568 | 0.0000983 | CD397710 | 508 | 43 | 86 | 50 | 1e−17 | response to UV |
| 3904 | 8.15322426 | down | 0.00000609 | 0.000102481 | BQ611037 | 600 | 80 | 194 | 41 | 3e−31 | regulation of transcription |
| 3905 | 8.148207088 | down | 2.11E−08 | 0.00000386 | BG237988 | 471 | 82 | 101 | 81 | 2e−44 | protein biosynthesis |
| 3906 | 8.137851814 | down | 0.0000647 | 0.000506814 | BM568005 | 418 | 9 | 14 | 64 | 8.7 | glycerol-3-phosphate metabolism |
| 3907 | 8.137850879 | down | 0.0000112 | 0.000151484 | BM107944 | 364 | 45 | 105 | 42 | 9e−23 | electron transport |
| 3908 | 8.131114565 | down | 3.82E−09 | 0.00000189 | CF807728 | 566 | 82 | 167 | 49 | 3e−42 | response to UV |
| 3909 | 8.11385643 | down | 1.06E−08 | 0.00000293 | U50075.1 | | | | | | Lipoxygenase L-5 (vlxB) |
| 3910 | 8.110774662 | down | 0.00000454 | 0.0000853 | BU546668 | 574 | 50 | 127 | 39 | 2e−18 | |
| 3911 | 8.093812534 | down | 0.00000562 | 0.0000975 | BQ453262 | 588 | 14 | 48 | 29 | 2.7 | transport |
| 3912 | 8.08871983 | down | 0.0000895 | 0.000636889 | CD394954 | 664 | 80 | 125 | 64 | 1e−40 | acetyl-CoA metabolism |
| 3913 | 8.087176248 | down | 0.0000219 | 0.000238908 | CF805734 | 561 | 156 | 187 | 83 | 1e−95 | |
| 3914 | 8.083023511 | down | 0.000000504 | 0.0000223 | AW734373 | 554 | 49 | 175 | 28 | 3e−13 | |
| 3915 | 8.075425981 | down | 0.000000158 | 0.0000112 | AW569918 | 467 | 86 | 105 | 81 | 2e−43 | |
| 3916 | 8.054695146 | down | 2.98E−09 | 0.00000172 | CD405936 | 349 | 12 | 15 | 80 | 0.004 | |
| 3917 | 8.033769655 | down | 0.000000405 | 0.0000195 | AW099756 | 410 | 66 | 83 | 79 | 4e−31 | |
| 3918 | 8.009846116 | down | 2.59E−08 | 0.00000425 | BQ610782 | 357 | 14 | 35 | 40 | 0.66 | proton transport |
| 3919 | 7.998671928 | down | 0.0000316 | 0.000309252 | BG789780 | | | | | | |
| 3920 | 7.903305692 | down | 0.000000168 | 0.0000117 | BQ742168 | 605 | 139 | 200 | 69 | 3e−75 | proline biosynthesis |
| 3921 | 7.89839282 | down | 0.000000709 | 0.0000276 | CD402282 | 679 | 107 | 130 | 82 | 3e−53 | nucleosome assembly |
| 3922 | 7.883877614 | down | 0.00000334 | 0.0000705 | BG237178 | | | | | | |
| 3923 | 7.877597566 | down | 3.06E−08 | 0.00000462 | CD403650 | 429 | 16 | 26 | 61 | 5e−04 | lipid transport |
| 3924 | 7.850510034 | down | 0.000000294 | 0.000016 | CD411430 | 699 | 48 | 91 | 52 | 3e−21 | regulation of transcription\, DNA- |
| | | | | | | | | | | | dependent |
| 3925 | 7.844584693 | down | 0.000000455 | 0.0000211 | BI943978 | 311 | 32 | 46 | 69 | 1e−17 | |
| 3926 | 7.833407152 | down | 0.00000249 | 0.0000587 | CD418606 | 291 | 15 | 58 | 25 | 5.4 | |
| 3927 | 7.831904785 | down | 0.00000026 | 0.0000148 | CD397710 | 508 | 43 | 86 | 50 | 1e−17 | response to UV |
| 3928 | 7.818853596 | down | 0.000000113 | 0.00000924 | CA935138 | 363 | 11 | 25 | 44 | 2.0 | |
| 3929 | 7.818310432 | down | 0.00000103 | 0.000034 | CF805814 | 483 | 24 | 64 | 37 | 0.004 | |
| 3930 | 7.806321486 | down | 0.000000359 | 0.0000181 | CD396052 | 555 | 55 | 87 | 63 | 3e−26 | |
| 3931 | 7.795286506 | down | 0.000000162 | 0.0000114 | AW184932 | 456 | 37 | 50 | 74 | 2e−17 | secondary cell wall biosynthesis (sensu |
| | | | | | | | | | | | Magnoliophyta) |
| 3932 | 7.788682637 | down | 0.000000257 | 0.0000148 | BE210278 | 349 | 13 | 24 | 54 | 1.1 | |
| 3933 | 7.767481006 | down | 0.00000581 | 0.0000994 | BE023603 | 428 | 68 | 132 | 51 | 2e−36 | |
| 3934 | 7.766157056 | down | 9.79E−08 | 0.00000853 | BE023121 | 435 | 75 | 143 | 52 | 3e−44 | |
| 3935 | 7.762029745 | down | 0.00000105 | 0.0000343 | BG510693 | 469 | 34 | 65 | 52 | 3e−14 | amino acid transport |
| 3936 | 7.744384868 | down | 0.00000015 | 0.000011 | AW349873 | 632 | 50 | 108 | 46 | 6e−20 | |
| 3937 | 7.744064326 | down | 0.000000156 | 0.0000112 | BE473800 | 457 | 57 | 141 | 40 | 4e−23 | protein amino acid phosphorylation |
| 3938 | 7.739189277 | down | 0.000001 | 0.0000337 | AW704742 | 351 | 90 | 117 | 76 | 8e−52 | tip growth |
| 3939 | 7.732330298 | down | 0.000000168 | 0.0000117 | BE821221 | 685 | 15 | 42 | 35 | 0.91 | |
| 3940 | 7.731377058 | down | 0.0000791 | 0.000582949 | U36442.1 | | | | | | lipoxygenase |
| 3941 | 7.730882882 | down | 1.63E−08 | 0.00000353 | CD393502 | 310 | 15 | 29 | 51 | 0.001 | |
| 3942 | 7.723242528 | down | 0.00010108 | 0.000692661 | L22162.1 | | | | | | brassinosteroid-regulated protein |
| 3943 | 7.716425751 | down | 3.25E−08 | 0.00000475 | BI973946 | 429 | 101 | 145 | 69 | 5e−56 | galactose metabolism |
| 3944 | 7.71578223 | down | 0.000387386 | 0.001825269 | BU547370 | 590 | 26 | 68 | 38 | 1e−08 | |
| 3945 | 7.713327803 | down | 0.00000281 | 0.000063 | BI315893 | 453 | 60 | 104 | 57 | 3e−31 | electron transport |
| 3946 | 7.699604442 | down | 9.38E−08 | 0.00000836 | BQ630544 | 636 | 154 | 210 | 73 | 1e−89 | carbohydrate metabolism |
| 3947 | 7.696022382 | down | 7.07E−08 | 0.00000729 | BI969418 | 522 | 64 | 87 | 73 | 2e−32 | carbohydrate metabolism |
| 3948 | 7.688454007 | down | 0.0000587 | 0.000473481 | BG041850 | 398 | 65 | 111 | 58 | 2e−38 | transport |
| 3949 | 7.669445084 | down | 0.000000738 | 0.0000283 | CF807456 | 788 | 183 | 262 | 69 | e−108 | |
| 3950 | 7.666964227 | down | 0.000000582 | 0.0000243 | BQ453599 | 382 | 14 | 23 | 60 | 0.009 | |
| 3951 | 7.662782833 | down | 0.000000052 | 0.00000617 | BE020127 | 592 | 137 | 157 | 87 | 2e−74 | Sali3-2, cell proliferation |
| 3952 | 7.633839987 | down | 0.000000416 | 0.0000199 | CD390951 | 620 | 15 | 19 | 78 | 0.16 | cell adhesion |
| 3953 | 7.621035223 | down | 0.0000104 | 0.000143546 | CF808798 | 548 | 92 | 137 | 67 | 2e−48 | beta-1,3-endoglucanase (EC 3.2.1.39), |
| | | | | | | | | | | | carbohydrate metabolism |
| 3954 | 7.620961368 | down | 1.81E−08 | 0.00000368 | BI969925 | 775 | 75 | 120 | 62 | 3e−37 | metabolism |
| 3955 | 7.616102117 | down | 0.000000453 | 0.000021 | AW309146 | 404 | 61 | 70 | 87 | 1e−31 | |
| 3956 | 7.612563067 | down | 0.00000281 | 0.000063 | CA783485 | 739 | 48 | 112 | 42 | 3e−16 | proteolysis and peptidolysis |
| 3957 | 7.606519838 | down | 0.0000148 | 0.000184055 | BI469627 | 431 | 36 | 48 | 75 | 2e−16 | |
| 3958 | 7.600619702 | down | 0.000000799 | 0.0000294 | CF809153 | 556 | 56 | 123 | 45 | 3e−20 | |
| 3959 | 7.595693295 | down | 0.0000042 | 0.0000812 | BG726168 | 508 | 142 | 166 | 85 | 2e−86 | cell elongation |
| 3960 | 7.584369199 | down | 0.000000501 | 0.0000222 | CA934978 | 301 | 20 | 35 | 57 | 8e−04 | |
| 3961 | 7.583353875 | down | 0.00000167 | 0.000045 | AW350602 | 736 | 97 | 171 | 56 | 3e−51 | carbohydrate metabolism |
| 3962 | 7.582635028 | down | 0.00000315 | 0.0000679 | BQ630307 | 555 | 109 | 169 | 64 | 7e−56 | |
| 3963 | 7.580829572 | down | 0.00000012 | 0.00000969 | CD399075 | 504 | 28 | 54 | 51 | 4e−10 | |
| 3964 | 7.572302558 | down | 1.11E−08 | 0.00000299 | BM269644 | 594 | 19 | 29 | 65 | 8e−08 | |
| 3965 | 7.568658107 | down | 0.000000132 | 0.0000102 | BG650527 | 412 | 73 | 96 | 76 | 4e−40 | proline biosynthesis |
| 3966 | 7.567993547 | down | 1.85E−08 | 0.00000371 | AW119494 | 425 | 57 | 78 | 73 | 2e−27 | glycoprotein catabolism |
| 3967 | 7.558666719 | down | 0.00000087 | 0.000031 | BU765440 | 353 | 16 | 34 | 47 | 0.39 | beta-1\, 3 glucan biosynthesis |
| 3968 | 7.548729872 | down | 0.000000558 | 0.0000239 | CD414564 | 309 | 13 | 22 | 59 | 0.84 | carbohydrate metabolism |
| 3969 | 7.538429172 | down | 0.000000151 | 0.000011 | AI443128 | 483 | 90 | 158 | 56 | 7e−48 | response to UV |
| 3970 | 7.529140873 | down | 8.79E−08 | 0.00000799 | BG042861 | 694 | 117 | 220 | 53 | 7e−63 | |
| 3971 | 7.502801501 | down | 0.00000456 | 0.0000855 | AJ293442.1 | | | | | | major latex protein homologue |
| 3972 | 7.48675361 | down | 0.00000596 | 0.000100889 | AI440768 | 409 | 98 | 134 | 73 | 6e−53 | proteolysis and peptidolysis |
| 3973 | 7.478635483 | down | 0.000000027 | 0.00000432 | CF805715 | 553 | 85 | 102 | 83 | 7e−42 | D-ribose metabolism |
| 3974 | 7.478266791 | down | 3.02E−08 | 0.00000046 | CF807624 | 710 | 119 | 188 | 63 | 4e−65 | proteolysis and peptidolysis |
| 3975 | 7.472385205 | down | 4.35E−08 | 0.00000553 | BM108115 | 592 | 10 | 24 | 41 | 0.92 | regulation of transcription\, DNA- |
| | | | | | | | | | | | dependent |
| 3976 | 7.467127809 | down | 0.000000571 | 0.0000242 | CD397710 | 508 | 43 | 86 | 50 | 1e−17 | response to UV |
| 3977 | 7.450163119 | down | 0.0000067 | 0.000108295 | BQ473377 | 580 | 33 | 105 | 31 | 3e−08 | |
| 3978 | 7.435373291 | down | 0.00000431 | 0.0000828 | BI971077 | 795 | 160 | 173 | 92 | 7e−91 | microtubule-based movement |
| 3979 | 7.432650177 | down | 0.000308674 | 0.001549923 | CA936141 | 363 | 21 | 42 | 50 | 2e−06 | |
| 3980 | 7.425915355 | down | 0.000000612 | 0.0000251 | AW203772 | 410 | 28 | 81 | 34 | 2e−05 | |
| 3981 | 7.424297809 | down | 0.0000393 | 0.00035746 | BI702268 | 380 | 26 | 31 | 83 | 2e−09 | |
| 3982 | 7.416469202 | down | 0.00000314 | 0.0000678 | BI786051 | 569 | 35 | 63 | 55 | 4e−13 | |
| 3983 | 7.414381652 | down | 3.42E−09 | 0.00000183 | BU765258 | 590 | 92 | 196 | 46 | 4e−49 | |
| 3984 | 7.410645999 | down | 0.000000766 | 0.0000289 | BE807671 | 563 | 136 | 193 | 70 | 2e−74 | carbohydrate metabolism |
| 3985 | 7.379563912 | down | 0.000000292 | 0.0000159 | BM954625 | 562 | 134 | 179 | 74 | 3e−82 | |
| 3986 | 7.369630654 | down | 0.00000027 | 0.000015 | BI969602 | 655 | 54 | 170 | 31 | 4e−07 | |
| 3987 | 7.35251794 | down | 0.00000582 | 0.0000994 | BU548459 | 496 | 39 | 110 | 35 | 2e−12 | |
| 3988 | 7.349150555 | down | 2.42E−08 | 0.00000413 | BE823255 | 605 | 75 | 142 | 52 | 3e−35 | |
| 3989 | 7.33875093 | down | 0.000125963 | 0.000813948 | BI943299 | 287 | 18 | 24 | 75 | 5e−04 | |
| 3990 | 7.336702652 | down | 0.000000184 | 0.0000123 | BI968419 | 788 | 69 | 157 | 43 | 1e−29 | protein amino acid phosphorylation |
| 3991 | 7.316703951 | down | 0.000000194 | 0.0000127 | BU764573 | 457 | 64 | 90 | 71 | 9e−34 | amino acid transport |
| 3992 | 7.315388118 | down | 0.000000416 | 0.0000199 | BQ628276 | 438 | 113 | 145 | 77 | 1e−65 | cation transport |
| 3993 | 7.312087106 | down | 0.0000744 | 0.000558607 | BE658341 | 449 | 65 | 98 | 66 | 6e−32 | response to oxidative stress |
| 3994 | 7.288800608 | down | 5.39E−09 | 0.00000216 | BU551192 | 602 | 59 | 125 | 47 | 3e−25 | proteolysis and peptidolysis |
| 3995 | 7.272549759 | down | 0.00000458 | 0.0000858 | CF921137 | 228 | 11 | 35 | 31 | 9.3 | |
| 3996 | 7.270772303 | down | 0.000100837 | 0.000691755 | AW310453 | 543 | 77 | 102 | 75 | 7e−40 | metabolism |
| 3997 | 7.26660897 | down | 0.000545528 | 0.002345619 | CF806531 | 509 | 106 | 168 | 63 | 4e−56 | nucleotide-sugar metabolism |
| 3998 | 7.262420924 | down | 0.000000719 | 0.0000279 | CA938490 | 380 | 19 | 39 | 48 | 2e−05 | |
| 3999 | 7.260606579 | down | 0.000209663 | 0.001166951 | BI968026 | 492 | 56 | 86 | 65 | 2e−29 | electron transport |
| 4000 | 7.249206087 | down | 9.42E−09 | 0.00000284 | BI967512 | 554 | 73 | 98 | 74 | 1e−36 | small GTPase mediated signal |
| | | | | | | | | | | | transduction |
| 4001 | 7.245826865 | down | 0.000000515 | 0.0000226 | BQ452617 | 360 | 46 | 71 | 64 | 1e−23 | |
| 4002 | 7.238672828 | down | 0.000000141 | 0.0000106 | BF068954 | 539 | 134 | 178 | 75 | 9e−80 | |
| 4003 | 7.22762777 | down | 2.06E−08 | 0.00000383 | AW310969 | 539 | 56 | 88 | 63 | 2e−31 | |
| 4004 | 7.192730011 | down | 0.00000544 | 0.0000955 | BQ297791 | 289 | 15 | 51 | 29 | 7.0 | |
| 4005 | 7.187168048 | down | 0.000000034 | 0.00000493 | BF069014 | 418 | 103 | 139 | 74 | 4e−59 | |
| 4006 | 7.179102213 | down | 0.00000269 | 0.0000614 | BU765404 | 603 | 116 | 157 | 73 | 5e−63 | electron transport |
| 4007 | 7.16579184 | down | 0.0000497 | 0.000420906 | BM522506 | | | | | | |
| 4008 | 7.160250422 | down | 0.00000155 | 0.000043 | AW458881 | 395 | 9 | 11 | 81 | 1.2 | |
| 4009 | 7.158791417 | down | 0.000537322 | 0.00231935 | BQ452690 | 528 | 107 | 135 | 79 | 7e−68 | |
| 4010 | 7.139244669 | down | 0.00000103 | 0.000034 | CD410684 | 369 | 13 | 18 | 72 | 0.11 | |
| 4011 | 7.133261273 | down | 0.00000129 | 0.0000382 | BI969859 | 692 | 60 | 77 | 77 | 8e−27 | |
| 4012 | 7.117973247 | down | 0.000000165 | 0.0000116 | CD418070 | 632 | 117 | 137 | 85 | 8e−65 | Cyclin-dependent kinases CDKB, |
| | | | | | | | | | | | regulation of mitotic cell cycle |
| 4013 | 7.117055448 | down | 0.000000629 | 0.0000255 | BQ611250 | 313 | 15 | 35 | 42 | 4.2 | transport |
| 4014 | 7.113034534 | down | 0.000000383 | 0.000019 | AW396070 | 637 | 65 | 210 | 30 | 4e−13 | |
| 4015 | 7.066653537 | down | 0.000000066 | 0.0000071 | CD403129 | 593 | 132 | 136 | 97 | 6e−69 | nucleosome assembly |
| 4016 | 7.059999029 | down | 4.98E−09 | 0.00000213 | AW348390 | 703 | 144 | 178 | 80 | 1e−82 | nucleotide-sugar metabolism |
| 4017 | 7.034911733 | down | 0.00000327 | 0.0000693 | BI970725 | 750 | 102 | 156 | 65 | 2e−53 | |
| 4018 | 7.029348174 | down | 7.56E−08 | 0.00000744 | BU549972 | 732 | 98 | 139 | 70 | 7e−60 | electron transport |
| 4019 | 7.023494319 | down | 0.0000269 | 0.000275977 | BU765780 | 421 | 62 | 136 | 45 | 2e−32 | electron transport |
| 4020 | 7.016766687 | down | 0.00000099 | 0.0000335 | BQ612445 | 613 | 164 | 206 | 79 | 2e−93 | secondary cell wall biosynthesis (sensu |
| | | | | | | | | | | | Magnoliophyta) |
| 4021 | 7.016728003 | down | 1.46E−08 | 0.00000347 | CD403261 | 602 | 33 | 91 | 36 | 5e−08 | response to auxin stimulus |
| 4022 | 7.000801664 | down | 0.0000102 | 0.000142185 | CF806282 | 650 | 76 | 132 | 57 | 3e−42 | |
| 4023 | 6.996029458 | down | 2.12E−08 | 0.00000386 | AW350946 | 623 | 45 | 94 | 47 | 5e−21 | carbohydrate metabolism |
| 4024 | 6.99479202 | down | 0.00000714 | 0.000112713 | AW350762 | 729 | 89 | 165 | 53 | 6e−44 | Putative NADPH:isoflavone reductase, |
| | | | | | | | | | | | nucleotide-sugar metabolism |
| 4025 | 6.969242132 | down | 0.00000571 | 0.0000985 | BG511704 | 314 | 20 | 75 | 26 | 0.034 | |
| 4026 | 6.968745914 | down | 7.42E−08 | 0.00000744 | CK605938 | 588 | 100 | 149 | 67 | 7e−60 | carbohydrate metabolism |
| 4027 | 6.938968747 | down | 0.00000102 | 0.0000339 | CF805750 | 543 | 57 | 177 | 32 | 2e−19 | cell wall modification |
| 4028 | 6.938495634 | down | 0.000122457 | 0.000795939 | BQ610288 | 441 | 15 | 35 | 42 | 7.6 | proteolysis and peptidolysis |
| 4029 | 6.913047959 | down | 0.0000761 | 0.000567269 | CD391559 | 661 | 97 | 152 | 63 | 1e−48 | transport |
| 4030 | 6.912691568 | down | 1.55E−08 | 0.00000353 | CA935397 | 356 | 12 | 30 | 40 | 1.9 | |
| 4031 | 6.906138681 | down | 0.001891321 | 0.005754432 | CD397262 | 438 | 38 | 61 | 62 | 2e−14 | metabolism |
| 4032 | 6.900075791 | down | 9.69E−08 | 0.00000847 | BQ452965 | 431 | 91 | 131 | 69 | 1e−49 | electron transport |
| 4033 | 6.888484836 | down | 0.0000011 | 0.0000351 | BU091077 | 423 | 17 | 48 | 35 | 8.9 | protein amino acid phosphorylation |
| 4034 | 6.8871533 | down | 0.000000229 | 0.0000139 | BE805868 | 550 | 103 | 154 | 66 | 2e−50 | |
| 4035 | 6.883412036 | down | 0.00000024 | 0.0000143 | BI317536 | 491 | 62 | 86 | 72 | 5e−30 | |
| 4036 | 6.872191182 | down | 0.00000323 | 0.0000688 | AW704362 | 563 | 141 | 193 | 73 | 4e−74 | |
| 4037 | 6.870166281 | down | 6.62E−08 | 0.0000071 | CD402735 | 661 | 56 | 75 | 74 | 3e−26 | D-xylose metabolism |
| 4038 | 6.8561861 | down | 0.0000452 | 0.000395134 | CD412716 | 655 | 11 | 38 | 28 | 1.4 | signal transduction |
| 4039 | 6.856006723 | down | 7.25E−09 | 0.00000235 | BQ454274 | 439 | 13 | 36 | 36 | 0.67 | |
| 4040 | 6.855524 | down | 2.66E−08 | 0.00000432 | CD406086 | 741 | 128 | 135 | 94 | 2e−72 | microtubule-based process |
| 4041 | 6.850647101 | down | 3.7E−09 | 0.00000188 | BG650239 | 415 | 12 | 18 | 66 | 0.45 | chitin catabolism |
| 4042 | 6.842750326 | down | 0.0000296 | 0.000294895 | CA784015 | 591 | 44 | 81 | 54 | 3e−17 | G-protein coupled receptor protein |
| | | | | | | | | | | | signaling pathway |
| 4043 | 6.831132913 | down | 0.00000308 | 0.0000669 | CF808930 | 696 | 195 | 231 | 84 | e−116 | cell elongation |
| 4044 | 6.81473665 | down | 0.000000303 | 0.0000164 | AB083030.1 | | | | | | syringolide-induced protein B15-3-5 |
| 4045 | 6.812014829 | down | 2.13E−08 | 0.00000386 | CK605690 | 576 | 73 | 135 | 54 | 3e−36 | response to oxidative stress |
| 4046 | 6.804798549 | down | 0.000000166 | 0.0000116 | BI424448 | 351 | 33 | 52 | 63 | 8e−18 | |
| 4047 | 6.791078111 | down | 0.000016 | 0.000193446 | CA800380 | 750 | 69 | 92 | 75 | 4e−35 | transport |
| 4048 | 6.789343093 | down | 9.26E−08 | 0.0000083 | BI702220 | 570 | 112 | 181 | 61 | 5e−62 | galactose metabolism |
| 4049 | 6.784053304 | down | 0.000481724 | 0.002140273 | BI321537 | 378 | 58 | 71 | 81 | 4e−27 | |
| 4050 | 6.777893681 | down | 0.000000221 | 0.0000137 | AW398009 | 306 | 15 | 51 | 29 | 3.2 | biological_process unknown |
| 4051 | 6.776568798 | down | 0.0000736 | 0.000554344 | BM121509 | 870 | 58 | 125 | 46 | 1e−26 | |
| 4052 | 6.768491534 | down | 0.000000549 | 0.0000236 | BG041563 | 247 | 44 | 51 | 86 | 2e−19 | aromatic compound metabolism |
| 4053 | 6.765416302 | down | 0.0000175 | 0.000205037 | CA799146 | 520 | 70 | 99 | 70 | 3e−38 | electron transport |
| 4054 | 6.752577526 | down | 0.000000257 | 0.0000148 | CD407893 | 634 | 82 | 179 | 45 | 2e−39 | |
| 4055 | 6.74697159 | down | 0.000252467 | 0.001336567 | BE821995 | 571 | 16 | 44 | 36 | 0.51 | |
| 4056 | 6.740575193 | down | 0.000000243 | 0.0000144 | AW423444 | 343 | 13 | 42 | 30 | 1.4 | |
| 4057 | 6.737035287 | down | 0.000000697 | 0.0000274 | L78163.1 | | | | | | peroxidase |
| 4058 | 6.726293728 | down | 2.82E−08 | 0.00000439 | BE822840 | 630 | 40 | 86 | 46 | 2e−13 | biological_process unknown |
| 4059 | 6.724976688 | down | 0.000000738 | 0.0000283 | BE820653 | 750 | 98 | 177 | 55 | 4e−50 | |
| 4060 | 6.710785342 | down | 0.000000224 | 0.0000138 | CF807282 | 488 | 45 | 79 | 56 | 1e−19 | |
| 4061 | 6.709139879 | down | 0.000000147 | 0.0000108 | CA935397 | 356 | 12 | 30 | 40 | 1.9 | |
| 4062 | 6.69699793 | down | 0.000000164 | 0.0000115 | AW830552 | 384 | 64 | 82 | 78 | 7e−34 | |
| 4063 | 6.693232974 | down | 0.00000729 | 0.000114115 | AF128268.1 | | | | | | trypsin inhibitor |
| 4064 | 6.689608052 | down | 0.000000871 | 0.000031 | BM085697 | 543 | 75 | 180 | 41 | 2e−33 | |
| 4065 | 6.683655194 | down | 0.000000585 | 0.0000244 | BM091989 | 301 | 11 | 34 | 32 | 0.85 | protein amino acid phosphorylation |
| 4066 | 6.678461154 | down | 0.00000183 | 0.000048 | BU763221 | 428 | 35 | 82 | 42 | 3e−13 | |
| 4067 | 6.675179098 | down | 0.000000006 | 0.00000221 | CK605685 | 268 | 11 | 29 | 37 | 5.5 | oligopeptide transport |
| 4068 | 6.663922074 | down | 0.00000369 | 0.0000751 | AW310119 | 470 | 12 | 32 | 37 | 0.21 | regulation of transcription\, DNA- |
| | | | | | | | | | | | dependent |
| 4069 | 6.659515924 | down | 0.000000357 | 0.000018 | BE821758 | 693 | 116 | 164 | 70 | 3e−65 | |
| 4070 | 6.655029424 | down | 0.00000101 | 0.0000338 | AW310932 | 424 | 11 | 28 | 39 | 3.0 | |
| 4071 | 6.654548526 | down | 0.00000308 | 0.0000669 | BM521730 | 586 | 149 | 192 | 77 | 3e−81 | cell elongation |
| 4072 | 6.65287177 | down | 0.00000559 | 0.0000972 | CD403463 | 583 | 41 | 74 | 55 | 3e−17 | response to auxin stimulus |
| 4073 | 6.639385893 | down | 3.15E−08 | 0.00000471 | BI470448 | 318 | 55 | 101 | 54 | 1e−27 | proteolysis and peptidolysis |
| 4074 | 6.631430483 | down | 0.0000102 | 0.000142234 | AW317461 | 305 | 45 | 67 | 67 | 3e−23 | proteolysis and peptidolysis |
| 4075 | 6.630262606 | down | 0.0000292 | 0.000291836 | AW101401 | 657 | 104 | 205 | 50 | 5e−46 | threonine catabolism |
| 4076 | 6.62398395 | down | 6.19E−08 | 0.00000682 | BM525054 | 442 | 11 | 23 | 47 | 7.6 | |
| 4077 | 6.604967412 | down | 0.00000058 | 0.0000243 | CA802874 | 367 | 13 | 47 | 27 | 2.8 | |
| 4078 | 6.59845995 | down | 0.000000238 | 0.0000142 | AW309422 | 576 | 80 | 107 | 74 | 8e−47 | |
| 4079 | 6.591052382 | down | 0.0000205 | 0.000227762 | BE658807 | 382 | 14 | 38 | 36 | 1.1 | |
| 4080 | 6.587312296 | down | 0.0000241 | 0.000256027 | BI970373 | 583 | 71 | 113 | 62 | 5e−38 | metabolism |
| 4081 | 6.582202968 | down | 0.000000797 | 0.0000294 | BG654242 | 397 | 11 | 16 | 68 | 1.5 | proteolysis and peptidolysis |
| 4082 | 6.576365983 | down | 0.00031987 | 0.001589219 | BU765089 | 408 | 46 | 109 | 42 | 3e−16 | |
| 4083 | 6.56215885 | down | 0.00000199 | 0.0000509 | BQ299690 | | | | | | |
| 4084 | 6.539768189 | down | 7.47E−08 | 0.00000744 | CF920436 | 662 | 80 | 120 | 66 | 2e−45 | |
| 4085 | 6.532515924 | down | 0.00000248 | 0.0000586 | BI699029 | 518 | 54 | 80 | 67 | 3e−25 | response to oxidative stress |
| 4086 | 6.530900167 | down | 0.00000882 | 0.000128459 | BI786385 | 561 | 62 | 79 | 78 | 4e−32 | |
| 4087 | 6.525513535 | down | 0.000000506 | 0.0000224 | BU548351 | 618 | 69 | 174 | 39 | 3e−29 | electron transport |
| 4088 | 6.517500716 | down | 4.82E−08 | 0.00000586 | CD392100 | 615 | 124 | 126 | 98 | 6e−65 | nucleosome assembly |
| 4089 | 6.513043434 | down | 0.000000269 | 0.000015 | BG507816 | 414 | 17 | 31 | 54 | 4e−05 | electron transport |
| 4090 | 6.498728211 | down | 1.53E−08 | 0.00000353 | CF805781 | 552 | 81 | 137 | 59 | 4e−42 | |
| 4091 | 6.49387735 | down | 0.00000219 | 0.000054 | BM568068 | 386 | 14 | 23 | 60 | 0.076 | |
| 4092 | 6.476027994 | down | 0.00000316 | 0.000068 | CA784872 | 422 | 62 | 130 | 47 | 4e−26 | cell adhesion |
| 4093 | 6.473873618 | down | 0.00000129 | 0.0000382 | CA819306 | 251 | 11 | 23 | 47 | 5.6 | regulation of transcription\, DNA- |
| | | | | | | | | | | | dependent |
| 4094 | 6.473257206 | down | 1.76E−08 | 0.00000368 | BG042866 | 456 | 18 | 58 | 31 | 0.43 | |
| 4095 | 6.463225291 | down | 0.000000218 | 0.0000137 | AW706246 | 359 | 18 | 53 | 33 | 0.89 | proteolysis and peptidolysis |
| 4096 | 6.46023507 | down | 4.83E−08 | 0.00000586 | AW423415 | 388 | 12 | 33 | 36 | 1.9 | |
| 4097 | 6.457057763 | down | 0.0000121 | 0.000159302 | BU550106 | 563 | 78 | 118 | 66 | 6e−42 | |
| 4098 | 6.455525094 | down | 9.58E−08 | 0.00000844 | CK606025 | 640 | 35 | 130 | 26 | 3e−05 | |
| 4099 | 6.430037357 | down | 0.0000726 | 0.000548296 | CA936141 | 363 | 21 | 42 | 50 | 2e−06 | |
| 4100 | 6.425773131 | down | 0.000000123 | 0.00000976 | BG551662 | 343 | 12 | 20 | 60 | 1.9 | mitotic cyclin /// mitotic cyclin |
| 4101 | 6.425747049 | down | 0.000155782 | 0.000942663 | AW309499 | 652 | 57 | 110 | 51 | 6e−25 | response to oxidative stress |
| 4102 | 6.42523594 | down | 0.00000066 | 0.0000264 | CD395891 | 551 | 75 | 90 | 83 | 3e−38 | Cyclin-dependent kinases CDKB, |
| | | | | | | | | | | | regulation of mitotic cell cycle |
| 4103 | 6.418047572 | down | 8.02E−09 | 0.00000253 | BQ576552 | 499 | 23 | 51 | 45 | 9e−06 | |
| 4104 | 6.41649134 | down | 8.62E−08 | 0.00000795 | BI968823 | 559 | 112 | 147 | 76 | 2e−61 | |
| 4105 | 6.415829519 | down | 0.000000248 | 0.0000145 | BE820838 | 610 | 65 | 144 | 45 | 2e−27 | protein amino acid phosphorylation |
| 4106 | 6.411562384 | down | 0.0000462 | 0.000402046 | CD412716 | 655 | 11 | 38 | 28 | 1.4 | signal transduction |
| 4107 | 6.405552405 | down | 0.00000182 | 0.0000478 | AW099942 | 684 | 128 | 208 | 61 | 1e−74 | proteolysis and peptidolysis |
| 4108 | 6.397672042 | down | 0.000000234 | 0.0000141 | BU084087 | 422 | 128 | 140 | 91 | 4e−69 | electron transport |
| 4109 | 6.387166323 | down | 0.00000011 | 0.00000914 | CD398819 | 687 | 126 | 163 | 77 | 6e−71 | nucleotide-sugar metabolism |
| 4110 | 6.386087705 | down | 0.000000255 | 0.0000148 | AW755915 | 442 | 76 | 154 | 49 | 3e−33 | cell wall modification |
| 4111 | 6.385546107 | down | 0.00000115 | 0.0000361 | AW349565 | 697 | 106 | 173 | 61 | 7e−63 | protein amino acid phosphorylation |
| 4112 | 6.382582239 | down | 0.000000544 | 0.0000235 | AW349775 | 637 | 46 | 77 | 59 | 5e−24 | proteolysis and peptidolysis |
| 4113 | 6.381899274 | down | 0.00000111 | 0.0000354 | AW100779 | 439 | 20 | 24 | 83 | 1e−04 | metabolism |
| 4114 | 6.36484046 | down | 0.0000167 | 0.000198771 | CK605906 | 793 | 85 | 140 | 60 | 6e−47 | |
| 4115 | 6.345652294 | down | 0.0000157 | 0.000191711 | BM269655 | 555 | 22 | 38 | 57 | 3e−07 | Putative 2-hydroxydihydrodaidzein |
| | | | | | | | | | | | reductase, anthocyanin biosynthesis |
| 4116 | 6.319712212 | down | 0.00000037 | 0.0000184 | BI970577 | 798 | 70 | 143 | 48 | 1e−30 | |
| 4117 | 6.299800159 | down | 0.0000125 | 0.000163493 | BQ079729 | 576 | 74 | 197 | 37 | 1e−27 | metabolism |
| 4118 | 6.297139164 | down | 0.000000135 | 0.0000104 | BE802171 | 411 | 16 | 49 | 32 | 2.9 | protein biosynthesis |
| 4119 | 6.293735916 | down | 0.00000351 | 0.0000725 | AI441660 | 444 | 74 | 104 | 71 | 5e−36 | |
| 4120 | 6.284793045 | down | 0.000000123 | 0.00000976 | CA783093 | 691 | 87 | 192 | 45 | 1e−38 | regulation of transcription\, DNA- |
| | | | | | | | | | | | dependent |
| 4121 | 6.281857956 | down | 0.00000218 | 0.0000538 | AJ563368.1 | | | | | | beta-fructofuranosidase |
| 4122 | 6.276976021 | down | 0.000000174 | 0.0000118 | CF808820 | 574 | 58 | 120 | 48 | 7e−27 | |
| 4123 | 6.267994648 | down | 0.000280081 | 0.001443343 | BU083449 | 363 | 11 | 34 | 32 | 2.7 | |
| 4124 | 6.259951213 | down | 0.000000294 | 0.000016 | BQ081656 | 327 | 15 | 65 | 23 | 7.0 | electron transport |
| 4125 | 6.254447193 | down | 4.38E−08 | 0.00000554 | CF805995 | 692 | 76 | 188 | 40 | 3e−38 | protein folding |
| 4126 | 6.246425767 | down | 0.00000228 | 0.0000554 | CF390492 | 642 | 78 | 105 | 74 | 2e−43 | |
| 4127 | 6.245996473 | down | 0.000000199 | 0.0000129 | CD417686 | 667 | 76 | 78 | 97 | 2e−36 | nucleosome assembly |
| 4128 | 6.241414963 | down | 0.00000634 | 0.000105046 | BE819933 | 551 | 29 | 34 | 85 | 5e−12 | |
| 4129 | 6.23205638 | down | 1.58E−08 | 0.00000353 | BU545899 | 515 | 12 | 20 | 60 | 0.72 | |
| 4130 | 6.227826299 | down | 0.00000049 | 0.000022 | BQ612494 | 290 | 12 | 13 | 92 | 0.37 | |
| 4131 | 6.223345863 | down | 7.31E−08 | 0.00000738 | CA852094 | 355 | 9 | 24 | 37 | 2.5 | metabolism |
| 4132 | 6.215905094 | down | 1.42E−08 | 0.00000341 | CD392332 | 604 | 34 | 38 | 89 | 2e−12 | |
| 4133 | 6.210696006 | down | 0.000000213 | 0.0000135 | CF806639 | 708 | 87 | 187 | 46 | 6e−38 | electron transport |
| 4134 | 6.21001537 | down | 0.000110592 | 0.000738804 | BI970784 | 714 | 83 | 110 | 75 | 2e−45 | carbohydrate metabolism |
| 4135 | 6.208031008 | down | 0.00000535 | 0.0000947 | AW307212 | 399 | 10 | 27 | 37 | 2.6 | |
| 4136 | 6.205681493 | down | 0.00000612 | 0.00010271 | BQ297915 | 583 | 95 | 110 | 86 | 7e−51 | galactose metabolism |
| 4137 | 6.193674916 | down | 0.000000279 | 0.0000155 | CA852607 | 412 | 15 | 39 | 38 | 1.7 | |
| 4138 | 6.182554995 | down | 0.00000159 | 0.0000439 | BE659231 | 485 | 41 | 66 | 62 | 1e−19 | sterol biosynthesis |
| 4139 | 6.181247376 | down | 0.00000117 | 0.0000363 | BE330624 | 389 | 74 | 114 | 64 | 5e−41 | DNA replication |
| 4140 | 6.177640793 | down | 0.000000527 | 0.000023 | CD402105 | 621 | 14 | 47 | 29 | 1.0 | |
| 4141 | 6.17518392 | down | 0.0000003 | 0.0000163 | BI426477 | 559 | 91 | 182 | 50 | 2e−50 | metabolism |
| 4142 | 6.031631045 | down | 0.00000274 | 0.000062 | CF809046 | 548 | 132 | 164 | 80 | 4e−69 | gravitropism |
| 4143 | 6.030929214 | down | 0.00000112 | 0.0000355 | AF228501.1 | | | | | | 14-3-3-like protein |
| 4144 | 6.027916041 | down | 0.000000532 | 0.0000231 | AI930728 | 578 | 133 | 167 | 79 | 2e−72 | glycoprotein catabolism |
| 4145 | 6.016468959 | down | 0.000000574 | 0.0000243 | CA819645 | 319 | 8 | 29 | 27 | 7.1 | protein folding |
| 4146 | 6.010105325 | down | 0.0000841 | 0.000608974 | AF195798.1 | | | | | | Isoflavone synthase 1 (ifs1) |
| 4147 | 6.009123638 | down | 0.0000099 | 0.00013935 | CD390408 | 640 | 15 | 26 | 57 | 0.011 | cell adhesion |
| 4148 | 6.003587334 | down | 0.00000171 | 0.0000459 | CA935147 | 252 | 10 | 24 | 41 | 1.9 | |
| 4149 | 5.997264693 | down | 0.00000105 | 0.0000343 | CD390492 | 642 | 78 | 105 | 74 | 2e−43 | |
| 4150 | 5.988147051 | down | 0.00000643 | 0.000105803 | BI971087 | 695 | 58 | 119 | 48 | 5e−28 | |
| 4151 | 5.986641652 | down | 0.000000496 | 0.0000221 | AW760844 | 421 | 37 | 95 | 38 | 1e−08 | |
| 4152 | 5.986420659 | down | 0.000438242 | 0.001995406 | BI317200 | 508 | 33 | 33 | 100 | 1e−12 | cation transport |
| 4153 | 5.985307626 | down | 0.00000823 | 0.000122544 | BM307261 | 611 | 62 | 150 | 41 | 1e−18 | |
| 4154 | 5.981175794 | down | 0.001075597 | 0.003844661 | CD405129 | 558 | 41 | 101 | 40 | 8e−18 | |
| 4155 | 5.978414029 | down | 0.000000347 | 0.0000177 | AW311001 | 679 | 86 | 125 | 68 | 1e−46 | |
| 4156 | 5.969624846 | down | 0.003517171 | 0.008901552 | BI424426 | 427 | 95 | 115 | 82 | 4e−51 | |
| 4157 | 5.957194742 | down | 0.000000194 | 0.0000127 | BF597401 | 414 | 34 | 65 | 52 | 2e−10 | morphogenesis |
| 4158 | 5.955129145 | down | 1.94E−08 | 0.00000376 | BI968591 | 775 | 164 | 190 | 86 | 1e−91 | |
| 4159 | 5.945757843 | down | 0.0000011 | 0.0000352 | BE657872 | 311 | 12 | 36 | 33 | 4.2 | transport |
| 4160 | 5.94488435 | down | 0.0000847 | 0.000612132 | CD406004 | 667 | 116 | 156 | 74 | 2e−68 | carbohydrate metabolism |
| 4161 | 5.932541819 | down | 0.000229916 | 0.001248204 | CD409747 | 461 | 17 | 49 | 34 | 0.018 | |
| 4162 | 5.930483029 | down | 0.0000691 | 0.000531083 | AI736154 | 349 | 10 | 27 | 37 | 9.3 | |
| 4163 | 5.926168617 | down | 0.00000521 | 0.0000932 | BF219543 | 582 | 69 | 96 | 71 | 2e−34 | |
| 4164 | 5.92148359 | down | 0.00000192 | 0.0000497 | CA784301 | 520 | 12 | 31 | 38 | 4.7 | DNA methylation |
| 4165 | 5.919958715 | down | 0.00000366 | 0.0000747 | BQ786484 | 426 | 100 | 101 | 99 | 1e−56 | microtubule-based process |
| 4166 | 5.907698793 | down | 0.000000131 | 0.0000102 | BU762121 | 592 | 76 | 143 | 53 | 4e−30 | ATP/ADP exchange |
| 4167 | 5.903842957 | down | 0.000000205 | 0.0000131 | BI785739 | 592 | 107 | 126 | 84 | 2e−62 | carbohydrate metabolism |
| 4168 | 5.90327821 | down | 0.0000185 | 0.000212611 | AI495276 | 350 | 71 | 104 | 68 | 1e−34 | |
| 4169 | 5.898194184 | down | 0.00000107 | 0.0000348 | CF808553 | 665 | 160 | 221 | 72 | 2e−92 | Sucrose synthase (SS), cell wall |
| | | | | | | | | | | | modification |
| 4170 | 5.895115886 | down | 0.00000575 | 0.0000986 | BQ298504 | 420 | 43 | 108 | 39 | 8e−17 | |
| 4171 | 5.884591416 | down | 0.000000617 | 0.0000252 | AI938331 | 221 | 11 | 26 | 42 | 3.2 | transport |
| 4172 | 5.883368824 | down | 0.00000388 | 0.0000773 | BQ079729 | 576 | 74 | 197 | 37 | 1e−27 | metabolism |
| 4173 | 5.876818685 | down | 0.000000219 | 0.0000137 | BU551023 | 624 | 49 | 206 | 23 | 2e−04 | metal ion transport |
| 4174 | 5.875540572 | down | 2.49E−08 | 0.00000414 | BQ627713 | 440 | 16 | 43 | 37 | 3.4 | carbohydrate metabolism |
| 4175 | 5.86506808 | down | 0.0000179 | 0.000207812 | AW705829 | | | | | | |
| 4176 | 5.863084899 | down | 0.000000574 | 0.0000243 | AW349515 | 662 | 101 | 124 | 81 | 1e−52 | |
| 4177 | 5.138084477 | down | 0.0000421 | 0.000375799 | BI787263 | 350 | 22 | 53 | 41 | 3e−05 | regulation of transcription |
| 4178 | 5.136541519 | down | 0.0000353 | 0.000333289 | CF807655 | 676 | 12 | 37 | 32 | 9.9 | metabolism |
| 4179 | 5.135851456 | down | 3.54E−08 | 0.00000499 | AW317636 | 511 | 64 | 99 | 64 | 3e−32 | |
| 4180 | 5.133669138 | down | 6.07E−08 | 0.00000678 | CD399083 | 676 | 90 | 139 | 64 | 3e−45 | |
| 4181 | 5.132736274 | down | 0.00000228 | 0.0000554 | BQ786519 | 555 | 111 | 140 | 79 | 2e−60 | protein amino acid phosphorylation |
| 4182 | 5.131950256 | down | 1.87E−08 | 0.00000371 | BU549650 | 450 | 28 | 65 | 43 | 4e−10 | xyloglucan biosynthesis |
| 4183 | 5.12581517 | down | 0.000000401 | 0.0000194 | BG507699 | 405 | 85 | 117 | 72 | 2e−47 | carbohydrate metabolism |
| 4184 | 5.124887568 | down | 8.26E−08 | 0.00000782 | BG045222 | 642 | 103 | 216 | 47 | 2e−57 | metabolism |
| 4185 | 5.11817814 | down | 0.000134239 | 0.000849473 | CF806068 | 548 | 55 | 89 | 61 | 3e−31 | response to gibberellic acid stimulus |
| 4186 | 5.117053692 | down | 4.14E−08 | 0.00000536 | CD405703 | 660 | 87 | 111 | 78 | 8e−50 | |
| 4187 | 5.113317899 | down | 8.11E−08 | 0.00000774 | BQ612873 | 619 | 152 | 205 | 74 | 7e−90 | protein amino acid phosphorylation |
| 4188 | 5.111747588 | down | 1.98E−08 | 0.00000378 | CF808617 | 533 | 33 | 85 | 38 | 2e−10 | |
| 4189 | 5.108611605 | down | 0.000011 | 0.000150039 | AI960156 | 641 | 108 | 135 | 80 | 1e−56 | malate metabolism |
| 4190 | 5.100228075 | down | 0.0000171 | 0.000201966 | AI960156 | 641 | 108 | 135 | 80 | 1e−56 | malate metabolism |
| 4191 | 5.093979257 | down | 0.000000212 | 0.0000135 | AW310236 | 414 | 36 | 63 | 57 | 1e−14 | |
| 4192 | 5.092537042 | down | 4.57E−08 | 0.00000565 | AW164593 | 308 | 17 | 53 | 32 | 0.65 | proteolysis and peptidolysis |
| 4193 | 5.088912925 | down | 0.000356334 | 0.001715932 | BQ453408 | 496 | 50 | 74 | 67 | 2e−23 | sodium ion transport |
| 4194 | 5.085558365 | down | 0.0000268 | 0.000275269 | BI470435 | 534 | 45 | 101 | 44 | 3e−20 | protein amino acid phosphorylation |
| 4195 | 5.081295702 | down | 0.000000059 | 0.00000668 | AW350127 | 662 | 98 | 179 | 54 | 2e−52 | beta-glucosidase, carbohydrate |
| | | | | | | | | | | | metabolism |
| 4196 | 5.080009904 | down | 0.000000345 | 0.0000177 | BM731862 | 535 | 85 | 113 | 75 | 1e−42 | jasmonic acid mediated signaling |
| | | | | | | | | | | | pathway (jasmonic acid/ethylene |
| | | | | | | | | | | | dependent systemic resistance) |
| 4197 | 5.072084919 | down | 0.000000394 | 0.0000192 | BM177229 | 405 | 57 | 135 | 42 | 4e−26 | |
| 4198 | 5.071366911 | down | 0.000000227 | 0.0000139 | CD400295 | 468 | 58 | 59 | 98 | 6e−27 | nucleosome assembly |
| 4199 | 5.067681827 | down | 0.000000265 | 0.0000149 | BM308215 | 460 | 82 | 149 | 55 | 4e−44 | electron transport |
| 4200 | 5.860695515 | down | 0.0000163 | 0.00019591 | BG238374 | 518 | 59 | 94 | 62 | 1e−31 | |
| 4201 | 5.857628944 | down | 0.000000824 | 0.00003 | BM885373 | 553 | 117 | 184 | 63 | 2e−67 | |
| 4202 | 5.856833891 | down | 0.000000967 | 0.000033 | BE822116 | 593 | 80 | 113 | 70 | 8e−43 | |
| 4203 | 5.85610483 | down | 0.0000131 | 0.000169399 | CA819120 | 442 | 18 | 50 | 36 | 1.5 | |
| 4204 | 5.853233248 | down | 4.16E−08 | 0.00000536 | BU550344 | 547 | 13 | 21 | 61 | 0.21 | |
| 4205 | 5.841749071 | down | 0.00000641 | 0.000105745 | BU548101 | 678 | 49 | 178 | 27 | 3e−17 | |
| 4206 | 5.839805096 | down | 0.0000555 | 0.00045205 | CF806068 | 548 | 55 | 89 | 61 | 3e−31 | response to gibberellic acid stimulus |
| 4207 | 5.819219015 | down | 0.00000095 | 0.0000326 | BQ612355 | 403 | 81 | 107 | 75 | 3e−47 | oligopeptide transport |
| 4208 | 5.814649134 | down | 0.000000111 | 0.0000092 | BG789774 | 409 | 38 | 47 | 80 | 2e−16 | |
| 4209 | 5.814461377 | down | 0.000000729 | 0.0000281 | BE330550 | 454 | 61 | 84 | 72 | 2e−29 | nucleotide-sugar metabolism |
| 4210 | 5.81220538 | down | 0.0000153 | 0.000188524 | AW706352 | 257 | 7 | 14 | 50 | 7.2 | electron transport |
| 4211 | 5.811752513 | down | 4.13E−08 | 0.00000536 | BE822050 | 638 | 76 | 134 | 56 | 4e−38 | |
| 4212 | 5.810705103 | down | 0.000000746 | 0.0000285 | AI442853 | 455 | 21 | 31 | 67 | 1e−04 | N-terminal protein myristoylation |
| 4213 | 5.809275519 | down | 0.0000035 | 0.0000725 | BF070320 | 639 | 77 | 122 | 63 | 3e−47 | |
| 4214 | 5.806769675 | down | 0.000000125 | 0.00000988 | BG406465 | | | | | | |
| 4215 | 5.795788921 | down | 0.000000201 | 0.0000129 | BI972166 | 420 | 22 | 103 | 21 | 2.3 | |
| 4216 | 5.79327977 | down | 0.0000196 | 0.000221197 | CD394606 | 592 | 111 | 136 | 81 | 3e−64 | regulation of transcription |
| 4217 | 5.787899622 | down | 0.000000334 | 0.0000173 | BU548303 | 600 | 51 | 89 | 57 | 4e−25 | |
| 4218 | 5.785854613 | down | 0.00000353 | 0.0000729 | AW394946 | 411 | 19 | 20 | 95 | 7e−05 | |
| 4219 | 5.78139954 | down | 0.000103976 | 0.000707453 | U89693.1 | | | | | | Sali3-2 |
| 4220 | 5.781055078 | down | 7.24E−08 | 0.00000737 | CD414114 | 650 | 87 | 135 | 64 | 9e−51 | |
| 4221 | 5.771959438 | down | 0.00000641 | 0.000105721 | AI759701 | 433 | 26 | 33 | 78 | 1e−09 | |
| 4222 | 5.771851016 | down | 0.000000367 | 0.0000184 | BQ740711 | 599 | 18 | 60 | 30 | 2.1 | protein amino acid phosphorylation |
| 4223 | 5.770218059 | down | 0.000000492 | 0.000022 | BI943706 | 518 | 89 | 157 | 56 | 5e−51 | |
| 4224 | 5.769800656 | down | 0.00003 | 0.000297721 | BG650228 | 239 | 13 | 14 | 92 | 0.004 | adaxial/abaxial pattern formation |
| 4225 | 5.767740714 | down | 0.0000137 | 0.000174054 | BU761052 | 526 | 94 | 175 | 53 | 4e−50 | |
| 4226 | 5.751168925 | down | 0.00000149 | 0.0000418 | CF922006 | 679 | 60 | 162 | 37 | 1e−33 | |
| 4227 | 5.751094162 | down | 0.00000415 | 0.0000805 | BG363518 | 480 | 67 | 106 | 63 | 2e−30 | proton transport |
| 4228 | 5.747521347 | down | 0.000000144 | 0.0000108 | AW760945 | 411 | 34 | 88 | 38 | 2e−09 | calnexin, sodium ion transport |
| 4229 | 5.733164484 | down | 0.000000673 | 0.0000268 | D50871.1 | | | | | | mitotic cyclin b1-type |
| 4230 | 5.731397886 | down | 0.000054 | 0.000446186 | AW432965 | 386 | 15 | 52 | 28 | 9.3 | oligosaccharide metabolism |
| 4231 | 5.730981984 | down | 3.89E−08 | 0.00000518 | BI971264 | 781 | 90 | 196 | 45 | 1e−41 | biosynthesis |
| 4232 | 5.730749189 | down | 0.000000952 | 0.0000327 | BF071699 | 592 | 151 | 201 | 75 | 1e−82 | protein amino acid phosphorylation |
| 4233 | 5.728493999 | down | 1.28E−08 | 0.00000319 | CD399924 | 482 | 55 | 73 | 75 | 1e−27 | jasmonic acid mediated signaling |
| | | | | | | | | | | | pathway (jasmonic acid/ethylene |
| | | | | | | | | | | | dependent systemic resistance) |
| 4234 | 5.725803139 | down | 0.0000001 | 0.0000086 | CF807687 | 764 | 87 | 236 | 36 | 8e−23 | |
| 4235 | 5.71803998 | down | 0.00000613 | 0.000102853 | CD417953 | 583 | 41 | 60 | 68 | 1e−20 | carbohydrate metabolism |
| 4236 | 5.71308163 | down | 0.0000251 | 0.000264072 | AW101759 | 375 | 38 | 67 | 56 | 2e−20 | |
| 4237 | 5.711614023 | down | 0.000000174 | 0.0000118 | BG156767 | 393 | 10 | 36 | 27 | 0.89 | transport |
| 4238 | 5.711456004 | down | 0.00000024 | 0.0000143 | AW706330 | 469 | 45 | 106 | 42 | 3e−15 | |
| 4239 | 5.705413606 | down | 0.0000455 | 0.000397757 | AI442650 | 533 | 49 | 107 | 45 | 1e−27 | |
| 4240 | 5.704460369 | down | 0.000000461 | 0.0000213 | CF808907 | 620 | 135 | 209 | 64 | 3e−78 | |
| 4241 | 5.702919483 | down | 0.0000043 | 0.0000827 | BU762621 | 577 | 58 | 176 | 32 | 7e−24 | |
| 4242 | 5.702710429 | down | 0.00000323 | 0.0000688 | BI967787 | 662 | 85 | 179 | 47 | 1e−39 | |
| 4243 | 5.696021234 | down | 0.0000624 | 0.000493869 | CF807760 | 412 | 52 | 96 | 54 | 3e−25 | |
| 4244 | 5.692248872 | down | 0.0000012 | 0.0000367 | AW309546 | 493 | 13 | 19 | 68 | 1.5 | protein amino acid phosphorylation |
| 4245 | 5.692185974 | down | 0.0000001 | 0.0000086 | BE823286 | 728 | 118 | 167 | 70 | 6e−62 | |
| 4246 | 5.689194215 | down | 6.99E−08 | 0.00000729 | BI967718 | 653 | 31 | 104 | 29 | 0.22 | |
| 4247 | 5.68497499 | down | 0.000000106 | 0.00000891 | AW349825 | 524 | 58 | 91 | 63 | 3e−30 | |
| 4248 | 5.684550127 | down | 0.000000208 | 0.0000133 | BE822014 | 566 | 58 | 75 | 77 | 2e−27 | |
| 4249 | 5.68296352 | down | 9.62E−08 | 0.00000844 | BQ742171 | 431 | 66 | 141 | 46 | 5e−31 | |
| 4250 | 5.68183558 | down | 0.00000526 | 0.0000936 | BG881338 | 348 | 20 | 66 | 30 | 0.22 | |
| 4251 | 5.680639724 | down | 0.00000146 | 0.0000413 | BG789619 | 336 | 10 | 32 | 31 | 5.5 | |
| 4252 | 5.671835486 | down | 0.00000462 | 0.000086 | AW394946 | 411 | 19 | 20 | 95 | 7e−05 | |
| 4253 | 5.665615022 | down | 0.000000638 | 0.0000257 | BU546595 | 648 | 47 | 117 | 40 | 1e−13 | |
| 4254 | 5.657481825 | down | 4.05E−08 | 0.00000531 | BG237670 | 333 | 12 | 36 | 33 | 1.5 | electron transport |
| 4255 | 5.657080935 | down | 0.0000001 | 0.0000086 | BQ612471 | 367 | 8 | 15 | 53 | 1.2 | |
| 4256 | 5.647958235 | down | 0.0000277 | 0.000281619 | AF128267.1 | | | | | | polygalacturonase PG2 |
| 4257 | 5.645025464 | down | 3.43E−08 | 0.00000493 | AW707266 | 371 | 16 | 26 | 61 | 2e−06 | |
| 4258 | 5.639763919 | down | 0.0000022 | 0.0000542 | AW570594 | 609 | 178 | 201 | 88 | 3e−99 | BiP isoform C |
| 4259 | 5.638037934 | down | 0.00000497 | 0.0000905 | BQ629010 | 266 | 20 | 30 | 66 | 1e−05 | response to pathogenic bacteria |
| | | | | | | | | | | | (incompatible interaction) |
| 4260 | 5.634589305 | down | 0.0000286 | 0.000288315 | BI426431 | 420 | 23 | 57 | 40 | 2e−07 | |
| 4261 | 5.629191238 | down | 0.000000537 | 0.0000233 | CF806590 | 668 | 161 | 222 | 72 | 2e−94 | calnexin, G-protein coupled receptor |
| | | | | | | | | | | | protein signaling pathway |
| 4262 | 5.626418717 | down | 0.00000113 | 0.0000358 | BG404902 | 406 | 73 | 115 | 63 | 1e−39 | |
| 4263 | 5.61763275 | down | 0.0000774 | 0.000573389 | AI495949 | 306 | 79 | 107 | 73 | 3e−37 | |
| 4264 | 5.616532232 | down | 0.0000127 | 0.000165664 | CF807229 | 644 | 79 | 213 | 37 | 1e−33 | |
| 4265 | 5.614630397 | down | 7.44E−08 | 0.00000744 | CA937343 | 346 | 12 | 29 | 41 | 5.5 | RNA dependent DNA replication |
| 4266 | 5.609081416 | down | 0.00000399 | 0.0000785 | BE657686 | 610 | 71 | 108 | 65 | 7e−36 | |
| 4267 | 5.607960638 | down | 0.000000297 | 0.0000161 | AW311298 | 652 | 119 | 219 | 54 | 4e−59 | |
| 4268 | 5.60357864 | down | 0.0000133 | 0.000170568 | BF008787 | 681 | 66 | 136 | 48 | 1e−34 | |
| 4269 | 5.59822558 | down | 0.00000029 | 0.0000159 | BQ081195 | 485 | 76 | 110 | 69 | 2e−40 | regulation of transcription |
| 4270 | 5.570898134 | down | 0.0000051 | 0.0000921 | BE657686 | 610 | 71 | 108 | 65 | 7e−36 | |
| 4271 | 5.567189004 | down | 0.000000171 | 0.0000117 | BE803404 | 517 | 115 | 172 | 66 | 6e−64 | biological_process unknown |
| 4272 | 5.56248369 | down | 7.97E−08 | 0.00000766 | BE805053 | 602 | 65 | 151 | 43 | 1e−27 | G-protein coupled receptor protein |
| | | | | | | | | | | | signaling pathway |
| 4273 | 5.544953456 | down | 0.0000238 | 0.000253843 | BI970840 | 691 | 68 | 149 | 45 | 4e−33 | response to oxidative stress |
| 4274 | 5.539737052 | down | 0.00000011 | 0.00000914 | CD418272 | 218 | 9 | 16 | 56 | 9.4 | |
| 4275 | 5.533082453 | down | 0.00000248 | 0.0000586 | BM893052 | 558 | 88 | 104 | 84 | 2e−47 | |
| 4276 | 5.531348305 | down | 0.000158025 | 0.000952558 | AW309671 | 632 | 72 | 108 | 66 | 2e−40 | |
| 4277 | 5.530976035 | down | 0.000000027 | 0.00000432 | CA852986 | 479 | 28 | 30 | 93 | 3e−10 | ciliary/flagellar motility |
| 4278 | 5.526618771 | down | 0.00000015 | 0.000011 | AW278755 | 651 | 167 | 217 | 76 | 3e−95 | cellulose biosynthesis |
| 4279 | 5.52629178 | down | 0.000003 | 0.0000659 | CD392693 | 626 | 34 | 130 | 26 | 0.011 | |
| 4280 | 5.522707658 | down | 0.00000055 | 0.0000236 | CD409235 | 605 | 37 | 46 | 80 | 1e−15 | protein amino acid phosphorylation |
| 4281 | 5.52051916 | down | 0.00000264 | 0.0000608 | BQ610347 | 345 | 25 | 67 | 37 | 3e−08 | electron transport |
| 4282 | 5.519713106 | down | 0.00000192 | 0.0000497 | BM178538 | 393 | 16 | 46 | 34 | 0.14 | regulation of translational initiation |
| 4283 | 5.518914592 | down | 7.57E−08 | 0.00000744 | BU550268 | 694 | 12 | 23 | 52 | 1.6 | |
| 4284 | 5.518636975 | down | 0.000000602 | 0.0000248 | AI442660 | 310 | 81 | 101 | 80 | 1e−45 | protein amino acid phosphorylation |
| 4285 | 5.515211767 | down | 0.00014969 | 0.000915071 | AI855972 | | | | | | |
| 4286 | 5.512186469 | down | 0.000000269 | 0.000015 | BU550745 | 591 | 88 | 167 | 52 | 2e−45 | |
| 4287 | 5.511584804 | down | 0.000000045 | 0.00000564 | BM522926 | 398 | 31 | 56 | 55 | 1e−12 | |
| 4288 | 5.503570334 | down | 0.000161048 | 0.000966907 | AW348608 | 563 | 93 | 150 | 62 | 4e−49 | |
| 4289 | 5.501416555 | down | 0.000000482 | 0.0000219 | AW349050 | 705 | 104 | 183 | 56 | 2e−57 | proteolysis and peptidolysis |
| 4290 | 5.487505051 | down | 0.000000315 | 0.0000167 | CD415666 | 561 | 57 | 75 | 76 | 2e−30 | electron transport |
| 4291 | 5.483262876 | down | 0.000000123 | 0.00000976 | CD409236 | 690 | 25 | 77 | 32 | 0.001 | |
| 4292 | 5.474040067 | down | 0.000000024 | 0.00000413 | BE020641 | 533 | 79 | 140 | 56 | 3e−41 | response to oxidative stress |
| 4293 | 5.473411084 | down | 0.000391919 | 0.001841323 | BQ613138 | 275 | 8 | 13 | 61 | 3.2 | |
| 4294 | 5.458051958 | down | 0.0000179 | 0.000207836 | AW351249 | 450 | 32 | 48 | 66 | 6e−13 | proteolysis and peptidolysis |
| 4295 | 5.454605418 | down | 0.000000168 | 0.0000117 | CA785641 | 592 | 91 | 195 | 46 | 2e−48 | carbohydrate metabolism |
| 4296 | 5.453243534 | down | 7.25E−08 | 0.00000737 | CD390580 | 488 | 20 | 61 | 32 | 0.85 | |
| 4297 | 5.450318986 | down | 0.00000178 | 0.000047 | BQ627749 | 179 | 10 | 22 | 45 | 4.2 | |
| 4298 | 5.445880575 | down | 0.000000391 | 0.0000191 | CD416791 | 586 | 19 | 59 | 32 | 1.2 | proteolysis and peptidolysis |
| 4299 | 5.443885677 | down | 1.05E−08 | 0.00000293 | BU549481 | 547 | 47 | 117 | 40 | 7e−19 | protein amino acid phosphorylation |
| 4300 | 5.440447575 | down | 0.00000784 | 0.000119244 | BI784811 | 283 | 12 | 19 | 63 | 0.37 | |
| 4301 | 5.432133361 | down | 0.000000366 | 0.0000184 | AW780843 | 194 | 13 | 37 | 35 | 1.9 | |
| 4302 | 5.408014176 | down | 0.0000354 | 0.000333289 | AW596467 | 493 | 129 | 163 | 79 | 3e−76 | carbohydrate metabolism |
| 4303 | 5.402853197 | down | 0.000000319 | 0.0000169 | BI971974 | 545 | 44 | 111 | 39 | 2e−12 | |
| 4304 | 5.402574556 | down | 0.00000178 | 0.0000472 | BF423817 | 405 | 15 | 42 | 35 | 6.1 | protein amino acid phosphorylation |
| 4305 | 5.3935316 | down | 0.00000267 | 0.0000612 | BE821285 | 459 | 41 | 83 | 49 | 8e−22 | |
| 4306 | 5.387719628 | down | 0.000000649 | 0.000026 | AW704807 | 378 | 14 | 57 | 24 | 3.9 | regulation of transcription\, DNA- |
| | | | | | | | | | | | dependent |
| 4307 | 5.378968709 | down | 0.00000162 | 0.0000442 | AW349529 | 611 | 39 | 76 | 51 | 2e−14 | response to dessication |
| 4308 | 5.3748195 | down | 0.000257449 | 0.001357634 | AI442942 | 500 | 14 | 51 | 27 | 0.048 | regulation of cell proliferation |
| 4309 | 5.367862931 | down | 2.09E−08 | 0.00000385 | BE802811 | 461 | 66 | 142 | 46 | 2e−30 | |
| 4310 | 5.365239744 | down | 0.00000116 | 0.0000363 | AW349408 | 562 | 22 | 63 | 34 | 0.38 | transport |
| 4311 | 5.361114935 | down | 0.00000205 | 0.0000517 | BF423685 | 415 | 96 | 141 | 68 | 3e−52 | |
| 4312 | 5.355464619 | down | 0.000000428 | 0.0000203 | CD404134 | 775 | 100 | 116 | 86 | 2e−54 | |
| 4313 | 5.354725093 | down | 0.00000265 | 0.0000609 | CF806273 | 449 | 12 | 21 | 57 | 2.1 | |
| 4314 | 5.351738195 | down | 0.000033 | 0.000318518 | CA784981 | 636 | 18 | 36 | 50 | 0.033 | |
| 4315 | 5.347832461 | down | 0.001377637 | 0.00458408 | AW349107 | 609 | 95 | 131 | 72 | 3e−50 | response to oxidative stress |
| 4316 | 5.345503191 | down | 0.000320654 | 0.001591018 | CA782249 | 267 | 15 | 37 | 40 | 7.2 | |
| 4317 | 5.345395465 | down | 0.000597062 | 0.002503389 | AF198453.1 | | | | | | UDP-glycose:flavonoid |
| | | | | | | | | | | | glycosyltransferase |
| 4318 | 5.340238584 | down | 0.000000174 | 0.0000118 | CK606142 | 797 | 217 | 256 | 84 | e−136 | |
| 4319 | 5.337964418 | down | 8.77E−08 | 0.00000799 | BI424855 | 437 | 36 | 82 | 43 | 3e−15 | |
| 4320 | 5.337643983 | down | 0.000000391 | 0.0000191 | BM527626 | 414 | 45 | 71 | 63 | 2e−22 | metabolism |
| 4321 | 5.333798173 | down | 2.86E−08 | 0.00000441 | AW349241 | 635 | 85 | 188 | 45 | 3e−34 | cytidine deamination |
| 4322 | 5.328674394 | down | 0.0000547 | 0.000450075 | BE823227 | 685 | 104 | 134 | 77 | 6e−53 | protein amino acid phosphorylation |
| 4323 | 5.32474607 | down | 2.79E−08 | 0.00000439 | U64916.1 | | | | | | GMFP5 |
| 4324 | 5.317591991 | down | 0.000000016 | 0.00000353 | BG789758 | 463 | 42 | 94 | 44 | 2e−21 | transport |
| 4325 | 5.30715083 | down | 0.000151275 | 0.000921618 | BE058056 | 271 | 12 | 21 | 57 | 9.3 | electron transport |
| 4326 | 5.303569976 | down | 0.000000752 | 0.0000286 | CF806876 | 656 | 92 | 146 | 63 | 6e−52 | |
| 4327 | 5.295994313 | down | 0.000000452 | 0.000021 | BM309768 | 565 | 161 | 188 | 85 | 1e−92 | boron transport |
| 4328 | 5.269969489 | down | 0.000000169 | 0.0000117 | AW704333 | 530 | 37 | 67 | 55 | 8e−17 | |
| 4329 | 5.265917979 | down | 0.000000764 | 0.0000289 | BG238111 | 276 | 16 | 42 | 38 | 0.099 | |
| 4330 | 5.262007086 | down | 0.000000144 | 0.0000108 | CD412608 | 671 | 95 | 138 | 68 | 3e−50 | |
| 4331 | 5.253872046 | down | 0.000000566 | 0.0000241 | CF805990 | 408 | 46 | 127 | 36 | 5e−15 | regulation of transcription |
| 4332 | 5.247154583 | down | 0.000000806 | 0.0000295 | BQ080473 | 514 | 70 | 125 | 56 | 2e−37 | carbohydrate metabolism |
| 4333 | 5.246974879 | down | 0.000000572 | 0.0000242 | BQ610840 | 514 | 85 | 110 | 77 | 2e−48 | |
| 4334 | 5.246513554 | down | 0.0000759 | 0.000566217 | BM187887 | 551 | 39 | 55 | 70 | 4e−15 | metal ion transport |
| 4335 | 5.24358071 | down | 0.000000924 | 0.0000321 | BU549268 | 629 | 69 | 79 | 87 | 5e−37 | |
| 4336 | 5.242039594 | down | 0.0000288 | 0.000289278 | BU765478 | 543 | 51 | 124 | 41 | 3e−15 | sodium ion transport |
| 4337 | 5.240657502 | down | 0.000000087 | 0.00000796 | BE821880 | 706 | 74 | 118 | 62 | 1e−36 | |
| 4338 | 5.24054772 | down | 0.00000277 | 0.0000622 | BI316840 | 379 | 60 | 126 | 47 | 9e−32 | metabolism |
| 4339 | 5.238520325 | down | 0.000000162 | 0.0000114 | CA820002 | 583 | 39 | 140 | 27 | 1e−04 | |
| 4340 | 5.23500347 | down | 5.22E−08 | 0.00000618 | BQ081542 | 558 | 124 | 154 | 80 | 9e−72 | acetyl-CoA biosynthesis |
| 4341 | 5.234966896 | down | 5.53E−08 | 0.00000644 | BM139673 | 421 | 14 | 41 | 34 | 3.0 | |
| 4342 | 5.232168224 | down | 0.0000647 | 0.000506763 | AW620528 | 544 | 94 | 167 | 56 | 4e−48 | |
| 4343 | 5.231801439 | down | 0.00000543 | 0.0000955 | BI968729 | 635 | 92 | 183 | 50 | 1e−46 | response to oxidative stress |
| 4344 | 5.227492437 | down | 0.000000909 | 0.0000318 | CD418191 | 345 | 16 | 23 | 69 | 0.003 | response to wounding |
| 4345 | 5.225053451 | down | 0.00000161 | 0.0000441 | BE658061 | 386 | 8 | 14 | 57 | 5.5 | transport |
| 4346 | 5.224964104 | down | 0.000000607 | 0.0000249 | AW100646 | 427 | 23 | 25 | 92 | 4e−08 | transport |
| 4347 | 5.222259436 | down | 0.0000482 | 0.000413158 | AI856426 | 579 | 84 | 135 | 62 | 2e−47 | |
| 4348 | 5.215400216 | down | 0.000000469 | 0.0000215 | CF808812 | 660 | 24 | 45 | 53 | 3e−05 | |
| 4349 | 5.20590331 | down | 0.00000183 | 0.000048 | AY204711.1 | | | | | | microsomal omega-3-fatty acid |
| | | | | | | | | | | | desaturase |
| 4350 | 5.203735204 | down | 0.001537096 | 0.004964055 | AW568912 | 415 | 28 | 37 | 75 | 2e−13 | proteolysis and peptidolysis |
| 4351 | 5.195639467 | down | 0.001387044 | 0.004606009 | BE658017 | 752 | 97 | 172 | 56 | 1e−50 | galactose metabolism |
| 4352 | 5.193525959 | down | 0.00000447 | 0.0000846 | BI470340 | 540 | 113 | 166 | 68 | 1e−60 | lipid metabolism |
| 4353 | 5.192060674 | down | 0.000000921 | 0.0000321 | BQ453384 | 382 | 21 | 37 | 56 | 3e−06 | |
| 4354 | 5.188922733 | down | 0.00000272 | 0.0000619 | BQ473162 | 442 | 9 | 21 | 42 | 9.9 | cell wall modification |
| 4355 | 5.18200742 | down | 2.56E−08 | 0.00000422 | CF807263 | 551 | 79 | 100 | 79 | 4e−46 | |
| 4356 | 5.181642297 | down | 0.000000178 | 0.000012 | BU544287 | 542 | 10 | 28 | 35 | 5.2 | |
| 4357 | 5.173638606 | down | 0.000000155 | 0.0000112 | CD413608 | 439 | 58 | 62 | 93 | 2e−28 | |
| 4358 | 5.165972611 | down | 0.000000184 | 0.0000123 | BU550344 | 547 | 13 | 21 | 61 | 0.21 | |
| 4359 | 5.152072454 | down | 0.000000213 | 0.0000135 | CK606714 | 185 | 8 | 17 | 47 | 7.1 | Cytosolic glutamine synthetase |
| | | | | | | | | | | | GSbeta 1 |
| 4360 | 5.144840072 | down | 0.0000208 | 0.000230316 | AW234261 | 405 | 88 | 131 | 67 | 3e−46 | response to oxidative stress |
| 4361 | 5.143114103 | down | 0.000031 | 0.000304634 | BQ629284 | 464 | 28 | 34 | 82 | 1e−10 | |
| 4362 | 5.142460837 | down | 0.0000051 | 0.0000921 | CF807323 | 751 | 73 | 163 | 44 | 2e−39 | response to pathogenic fungi |
| 4363 | 5.140752217 | down | 0.00000253 | 0.0000594 | CD405657 | 308 | 11 | 36 | 30 | 0.22 | DNA repair |
| 4364 | 5.1389832 | down | 0.000268184 | 0.001396964 | CF806168 | 397 | 77 | 108 | 71 | 1e−38 | response to oxidative stress |
| 4365 | 5.063653929 | down | 0.000000433 | 0.0000205 | CD391349 | 693 | 88 | 137 | 64 | 5e−47 | |
| 4366 | 5.062679642 | down | 0.000000599 | 0.0000248 | CA936335 | 455 | 94 | 149 | 63 | 5e−51 | proton transport |
| 4367 | 5.057940466 | down | 0.000000018 | 0.00000368 | AF357211.1 | | | | | | ethylene-responsive element binding |
| | | | | | | | | | | | protein 1 |
| 4368 | 5.05542808 | down | 3.88E−08 | 0.00000518 | BG238698 | 449 | 20 | 70 | 28 | 0.71 | regulation of transcription\, DNA- |
| | | | | | | | | | | | dependent |
| 4369 | 5.055051265 | down | 0.000016 | 0.000193845 | BI317200 | 508 | 33 | 33 | 100 | 1e−12 | cation transport |
| 4370 | 5.0484233 | down | 0.0000132 | 0.000170001 | CF805870 | 678 | 192 | 224 | 85 | e−113 | sucrose synthase, response to cold |
| 4371 | 5.047385039 | down | 0.000000552 | 0.0000236 | BI317002 | 445 | 17 | 59 | 28 | 2.6 | |
| 4372 | 5.046440783 | down | 3.81E−08 | 0.00000515 | AW598590 | | | | | | |
| 4373 | 5.041491712 | down | 0.00001 | 0.000140914 | CF809316 | 757 | 161 | 251 | 64 | 7e−95 | |
| 4374 | 5.037341574 | down | 0.00000112 | 0.0000355 | CA799081 | 566 | 127 | 189 | 67 | 2e−74 | |
| 4375 | 5.030811321 | down | 0.000127191 | 0.000818504 | AW666270 | | | | | | |
| 4376 | 5.026609387 | down | 0.000318805 | 0.00158681 | AW705639 | 408 | 85 | 133 | 63 | 2e−44 | |
| 4377 | 5.024715309 | down | 2.42E−08 | 0.00000413 | BU547877 | 670 | 128 | 182 | 70 | 2e−77 | carbohydrate metabolism |
| 4378 | 5.023394498 | down | 0.00000149 | 0.0000419 | CD390523 | 458 | 39 | 58 | 67 | 1e−16 | proteolysis and peptidolysis |
| 4379 | 5.02329606 | down | 0.000000851 | 0.0000306 | CD394817 | 634 | 49 | 87 | 56 | 3e−17 | |
| 4380 | 5.022876136 | down | 0.000011 | 0.000149599 | CD397706 | 359 | 29 | 33 | 87 | 5e−13 | regulation of transcription |
| 4381 | 5.02168752 | down | 0.00000172 | 0.0000461 | AW100736 | 694 | 64 | 95 | 67 | 2e−32 | |
| 4382 | 5.020842512 | down | 0.00000025 | 0.0000146 | BE021748 | 415 | 10 | 32 | 31 | 3.8 | |
| 4383 | 5.016240592 | down | 0.00000668 | 0.000108177 | BE659297 | 385 | 30 | 45 | 66 | 7e−11 | electron transport |
| 4384 | 4.997883072 | down | 0.00000794 | 0.000120147 | CF806892 | 678 | 134 | 224 | 59 | 1e−71 | proteolysis and peptidolysis |
| 4385 | 4.990810853 | down | 0.000000108 | 0.00000905 | CF808173 | 612 | 46 | 83 | 55 | 5e−21 | protein amino acid phosphorylation |
| 4386 | 4.988597191 | down | 0.000000244 | 0.0000144 | BI942329 | 174 | 10 | 30 | 33 | 1.9 | |
| 4387 | 4.975432246 | down | 0.000000671 | 0.0000267 | CF806381 | 690 | 186 | 211 | 88 | e−103 | |
| 4388 | 4.972830756 | down | 9.87E−08 | 0.00000857 | AW311553 | 282 | 13 | 47 | 27 | 1.9 | |
| 4389 | 4.971092788 | down | 0.00000021 | 0.0000134 | BI321105 | 570 | 62 | 184 | 33 | 6e−23 | |
| 4390 | 4.970611146 | down | 0.00000296 | 0.0000652 | AW307307 | 321 | 16 | 48 | 33 | 2.4 | |
| 4391 | 4.958353879 | down | 0.00000202 | 0.0000512 | CF808820 | 574 | 58 | 120 | 48 | 7e−27 | |
| 4392 | 4.955623975 | down | 0.00019253 | 0.001095584 | BU548731 | 644 | 53 | 110 | 48 | 3e−16 | G-protein coupled receptor protein |
| | | | | | | | | | | | signaling pathway |
| 4393 | 4.944755912 | down | 0.00000106 | 0.0000344 | CF806819 | 650 | 101 | 181 | 55 | 3e−55 | |
| 4394 | 4.944734247 | down | 0.00000162 | 0.0000442 | BQ611829 | 520 | 85 | 178 | 47 | 8e−29 | regulation of transcription\, DNA- |
| | | | | | | | | | | | dependent |
| 4395 | 4.941954279 | down | 0.00000541 | 0.0000953 | CD402237 | 511 | 70 | 83 | 84 | 7e−36 | DNA replication initiation |
| 4396 | 4.939964431 | down | 0.0000304 | 0.00030097 | AF243367.1 | | | | | | glutathione S-transferase GST 12 |
| 4397 | 4.93726129 | down | 0.000000831 | 0.0000302 | BQ296088 | 581 | 110 | 194 | 56 | 1e−55 | G-protein coupled receptor protein |
| | | | | | | | | | | | signaling pathway |
| 4398 | 4.936073089 | down | 0.0000053 | 0.0000942 | CD406505 | 501 | 11 | 19 | 57 | 1.2 | |
| 4399 | 4.935224172 | down | 0.000000842 | 0.0000304 | BI701092 | 492 | 57 | 126 | 45 | 8e−18 | |
| 4400 | 4.930118954 | down | 0.000963941 | 0.003558162 | AW350431 | 766 | 103 | 183 | 56 | 1e−54 | |
| 4401 | 4.929153325 | down | 0.000000218 | 0.0000137 | CD403154 | 614 | 93 | 96 | 96 | 1e−46 | nucleosome assembly |
| 4402 | 4.928675832 | down | 0.0000401 | 0.000362504 | BG789780 | | | | | | |
| 4403 | 4.926097002 | down | 0.0000011 | 0.0000351 | BM522504 | 469 | 34 | 93 | 36 | 6e−11 | |
| 4404 | 4.91994933 | down | 0.00000369 | 0.0000751 | CD405147 | 627 | 72 | 86 | 83 | 8e−41 | |
| 4405 | 4.917991204 | down | 0.0000168 | 0.000199563 | CA801519 | 408 | 20 | 64 | 31 | 0.33 | |
| 4406 | 4.910506535 | down | 0.0000125 | 0.00016339 | BQ629293 | 431 | 40 | 97 | 41 | 3e−17 | |
| 4407 | 4.910084445 | down | 0.000000194 | 0.0000127 | BE805868 | 550 | 103 | 154 | 66 | 2e−50 | |
| 4408 | 4.907155818 | down | 0.000235533 | 0.0012715 | BI970958 | 789 | 97 | 233 | 41 | 4e−46 | metabolism |
| 4409 | 4.904252701 | down | 0.0000354 | 0.000333914 | BI970389 | 652 | 90 | 161 | 55 | 2e−47 | |
| 4410 | 4.903803962 | down | 0.00000306 | 0.0000667 | BE801366 | 481 | 40 | 68 | 58 | 4e−15 | response to light |
| 4411 | 4.898674314 | down | 0.0000111 | 0.000151387 | CD395459 | 358 | 17 | 75 | 22 | 0.86 | |
| 4412 | 4.898629714 | down | 0.000000329 | 0.0000172 | AF516879.1 | | | | | | expansin |
| 4413 | 4.896704277 | down | 0.00000102 | 0.0000339 | CA802402 | 277 | 8 | 20 | 40 | 3.2 | transport |
| 4414 | 4.89652659 | down | 0.000000861 | 0.0000309 | BE330621 | 667 | 139 | 198 | 70 | 7e−78 | carbohydrate metabolism |
| 4415 | 4.895233089 | down | 0.00000255 | 0.0000596 | BU548143 | 632 | 91 | 145 | 62 | 2e−47 | |
| 4416 | 4.894996936 | down | 0.000262931 | 0.001376848 | CD395463 | 345 | 15 | 38 | 39 | 3.2 | nucleobase\, nucleoside\, nucleotide |
| | | | | | | | | | | | and nucleic acid transport |
| 4417 | 4.888755656 | down | 0.00000749 | 0.000116042 | BI971040 | 798 | 150 | 198 | 75 | 2e−87 | beta-D-glucan exohydrolase, |
| | | | | | | | | | | | carbohydrate metabolism |
| 4418 | 4.888088692 | down | 0.00000337 | 0.0000708 | CD398711 | 496 | 53 | 74 | 71 | 4e−32 | carbohydrate metabolism |
| 4419 | 4.88679575 | down | 0.00000948 | 0.000134994 | BQ452657 | 588 | 69 | 72 | 95 | 1e−34 | biological_process unknown |
| 4420 | 4.884616268 | down | 0.0000213 | 0.000234181 | CD407325 | 628 | 125 | 156 | 80 | 2e−73 | proteolysis and peptidolysis |
| 4421 | 4.883698151 | down | 0.000000477 | 0.0000217 | BE822676 | 511 | 55 | 139 | 39 | 6e−24 | antibiotic biosynthesis |
| 4422 | 4.882471113 | down | 2.23E−08 | 0.00000396 | AI443042 | 532 | 104 | 128 | 81 | 4e−51 | flavonoid biosynthesis |
| 4423 | 4.87953406 | down | 0.000000171 | 0.0000117 | AI441809 | 393 | 71 | 95 | 74 | 1e−41 | carbohydrate metabolism |
| 4424 | 4.877929366 | down | 0.0000145 | 0.00018162 | BQ296859 | 581 | 134 | 194 | 69 | 3e−75 | electron transport |
| 4425 | 4.861210723 | down | 0.000000147 | 0.0000108 | CA800410 | 596 | 84 | 158 | 53 | 2e−47 | carbohydrate metabolism |
| 4426 | 4.856705713 | down | 0.00018071 | 0.001046212 | BE347412 | 468 | 89 | 145 | 61 | 6e−47 | sulfate transport |
| 4427 | 4.854183279 | down | 0.0000768 | 0.0005702 | BG882393 | 430 | 11 | 28 | 39 | 0.009 | |
| 4428 | 4.853783083 | down | 0.0000383 | 0.000352083 | AJ563383.1 | | | | | | ornithine decarboxylase |
| 4429 | 4.846333813 | down | 0.00000685 | 0.000109774 | AW472464 | 618 | 124 | 190 | 65 | 3e−65 | |
| 4430 | 4.836179313 | down | 0.000113363 | 0.000751372 | BQ785704 | 371 | 9 | 19 | 47 | 1.3 | |
| 4431 | 4.83565526 | down | 0.000071 | 0.000541 | AI416749 | 268 | 13 | 22 | 59 | 3.2 | |
| 4432 | 4.83516736 | down | 0.000000262 | 0.0000149 | BE659836 | 571 | 49 | 82 | 59 | 8e−20 | |
| 4433 | 4.833271373 | down | 0.000000577 | 0.0000243 | CD402480 | 495 | 43 | 52 | 82 | 1e−17 | |
| 4434 | 4.828420318 | down | 0.000000349 | 0.0000178 | BI471498 | 575 | 112 | 191 | 58 | 6e−64 | |
| 4435 | 4.827984305 | down | 0.000000396 | 0.0000192 | BG882734 | 528 | 26 | 66 | 39 | 7e−05 | |
| 4436 | 4.825707416 | down | 0.0000019 | 0.0000493 | AW164216 | 638 | 165 | 210 | 78 | 4e−99 | carbohydrate metabolism |
| 4437 | 4.818492331 | down | 0.0000217 | 0.00023715 | CA936403 | 591 | 36 | 81 | 44 | 2e−15 | response to dessication |
| 4438 | 4.818080634 | down | 2.31E−08 | 0.00000404 | BE820625 | 737 | 162 | 192 | 84 | 3e−92 | nucleotide-sugar metabolism |
| 4439 | 4.815313336 | down | 0.000000187 | 0.0000124 | BG511492 | 248 | 14 | 50 | 28 | 0.63 | |
| 4440 | 4.813551301 | down | 0.000000416 | 0.0000199 | BG551662 | 343 | 12 | 20 | 60 | 1.9 | mitotic cyclin |
| 4441 | 4.813390234 | down | 0.00000497 | 0.0000905 | BE555169 | 426 | 64 | 87 | 73 | 1e−30 | secondary cell wall biosynthesis (sensu |
| | | | | | | | | | | | Magnoliophyta) |
| 4442 | 4.81065176 | down | 1.63E−08 | 0.00000353 | BI701165 | 545 | 36 | 113 | 31 | 9e−05 | response to water deprivation |
| 4443 | 4.806215468 | down | 0.0000112 | 0.000151484 | BU549591 | 456 | 39 | 44 | 88 | 2e−17 | |
| 4444 | 4.80538976 | down | 1.66E−08 | 0.00000356 | BE822954 | 489 | 77 | 110 | 70 | 1e−35 | galactose metabolism |
| 4445 | 4.8050872 | down | 0.00000875 | 0.000127787 | AW666288 | 573 | 172 | 188 | 91 | 7e−97 | |
| 4446 | 4.802481538 | down | 0.00000203 | 0.0000515 | AW704315 | 602 | 93 | 116 | 80 | 3e−49 | protein amino acid phosphorylation |
| 4447 | 4.802247038 | down | 0.00000835 | 0.000123959 | BQ080157 | 218 | 13 | 32 | 40 | 7.2 | |
| 4448 | 4.797756555 | down | 0.0000101 | 0.000141229 | CD401630 | 618 | 37 | 70 | 52 | 2e−16 | proteolysis and peptidolysis |
| 4449 | 4.796452657 | down | 0.000000103 | 0.00000878 | AW310211 | 665 | 80 | 155 | 51 | 4e−39 | |
| 4450 | 4.786899496 | down | 0.0000762 | 0.000567269 | CF806057 | 510 | 44 | 72 | 61 | 3e−20 | |
| 4451 | 4.781465105 | down | 0.000000468 | 0.0000214 | CK606142 | 797 | 217 | 256 | 84 | e−136 | |
| 4452 | 4.779241025 | down | 0.0000131 | 0.000169289 | BM523674 | 168 | 9 | 27 | 33 | 5.5 | |
| 4453 | 4.774346507 | down | 0.000220611 | 0.001212106 | BI784696 | 399 | 16 | 42 | 38 | 0.082 | signal transduction |
| 4454 | 4.767456086 | down | 0.0000101 | 0.000141229 | BI321021 | 233 | 48 | 77 | 62 | 1e−19 | |
| 4455 | 4.767385876 | down | 0.00000609 | 0.000102481 | AW349068 | 655 | 66 | 120 | 55 | 2e−38 | |
| 4456 | 4.760722283 | down | 4.17E−08 | 0.00000536 | CD401652 | 480 | 26 | 67 | 38 | 4e−10 | glutathione S-transferase GST 17, |
| | | | | | | | | | | | aromatic amino acid family metabolism |
| 4457 | 4.752943977 | down | 3.74E−08 | 0.00000512 | BG041904 | 533 | 97 | 172 | 56 | 3e−57 | |
| 4458 | 4.751532721 | down | 0.00000802 | 0.000120862 | BE330184 | 597 | 127 | 194 | 65 | 5e−73 | |
| 4459 | 4.746861403 | down | 0.00000103 | 0.0000339 | BQ610840 | 514 | 85 | 110 | 77 | 2e−48 | |
| 4460 | 4.74148909 | down | 0.0000156 | 0.000190395 | BQ453594 | 420 | 21 | 68 | 30 | 1.7 | regulation of transcription |
| 4461 | 4.738282354 | down | 0.0000497 | 0.000420922 | CF921337 | 622 | 51 | 121 | 42 | 5e−21 | |
| 4462 | 4.735729988 | down | 0.00000588 | 0.000100049 | AI856145 | 264 | 53 | 69 | 76 | 4e−26 | |
| 4463 | 4.734602833 | down | 0.000000768 | 0.000029 | BI700722 | 583 | 72 | 179 | 40 | 6e−34 | metabolism |
| 4464 | 4.73148754 | down | 7.05E−08 | 0.00000729 | BE820147 | 285 | 8 | 28 | 28 | 9.2 | |
| 4465 | 4.718859418 | down | 0.000000605 | 0.0000249 | BU763259 | 593 | 94 | 112 | 83 | 3e−54 | |
| 4466 | 4.718192272 | down | 0.00000851 | 0.000125325 | CD398677 | 448 | 44 | 88 | 50 | 2e−18 | response to pathogenic bacteria |
| | | | | | | | | | | | (incompatible interaction) |
| 4467 | 4.714593442 | down | 0.000000398 | 0.0000193 | AW348133 | 445 | 34 | 56 | 60 | 8e−10 | |
| 4468 | 4.71000163 | down | 0.000000943 | 0.0000325 | BE609308 | 383 | 13 | 31 | 41 | 4.1 | |
| 4469 | 4.707525131 | down | 0.00000229 | 0.0000555 | AW396216 | 440 | 24 | 33 | 72 | 2e−08 | |
| 4470 | 4.705873138 | down | 0.00000218 | 0.0000538 | CA936959 | 449 | 30 | 35 | 85 | 3e−12 | |
| 4471 | 4.705056216 | down | 1.95E−08 | 0.00000376 | CF809070 | 668 | 132 | 176 | 75 | 6e−85 | chitin catabolism |
| 4472 | 4.70428711 | down | 0.000000353 | 0.0000179 | BM523606 | 545 | 80 | 172 | 46 | 2e−40 | |
| 4473 | 4.692327505 | down | 5.58E−08 | 0.00000648 | CF806561 | 676 | 115 | 226 | 50 | 2e−60 | |
| 4474 | 4.690947226 | down | 0.000000949 | 0.0000326 | BU549947 | 677 | 120 | 163 | 73 | 1e−65 | UDP-glucose metabolism |
| 4475 | 4.687930771 | down | 0.00000142 | 0.0000406 | CD406270 | 575 | 67 | 88 | 76 | 2e−34 | |
| 4476 | 4.687566663 | down | 0.00035001 | 0.001692908 | BM885487 | 372 | 12 | 36 | 33 | 3.8 | |
| 4477 | 4.687459691 | down | 0.00000111 | 0.0000354 | BE555263 | 474 | 69 | 138 | 50 | 8e−35 | transport |
| 4478 | 4.686222112 | down | 0.000200746 | 0.001131048 | CD396846 | 711 | 70 | 145 | 48 | 3e−35 | response to pathogenic fungi |
| 4479 | 4.685690951 | down | 0.00000561 | 0.0000975 | BF070693 | 337 | 65 | 111 | 58 | 6e−29 | electron transport |
| 4480 | 4.6849307 | down | 0.000000652 | 0.0000261 | BI970329 | 679 | 71 | 139 | 51 | 3e−35 | metabolism |
| 4481 | 4.683956521 | down | 0.00000489 | 0.0000894 | BG725476 | 393 | 58 | 135 | 42 | 6e−26 | |
| 4482 | 4.683843393 | down | 4.97E−08 | 0.00000596 | BU548256 | 626 | 97 | 129 | 75 | 6e−59 | lipid catabolism |
| 4483 | 4.682543261 | down | 0.003272532 | 0.00846582 | CD400221 | 415 | 13 | 44 | 29 | 0.59 | Homeodomain-leucine zipper protein 56 |
| | | | | | | | | | | | (Hdl56) |
| 4484 | 4.677046602 | down | 5.63E−08 | 0.00000652 | BE658830 | 578 | 10 | 22 | 45 | 10.0 | |
| 4485 | 4.674213463 | down | 0.00000114 | 0.000036 | CA852787 | 332 | 22 | 23 | 95 | 5e−06 | |
| 4486 | 4.673066828 | down | 0.00000152 | 0.0000424 | CF921991 | 539 | 82 | 82 | 100 | 1e−40 | nucleosome assembly |
| 4487 | 4.670676548 | down | 0.00000114 | 0.0000359 | BU544394 | 617 | 43 | 83 | 51 | 4e−10 | DNA methylation |
| 4488 | 4.664185165 | down | 0.001031602 | 0.003728518 | U30475.1 | | | | | | homeobox-leucine zipper protein |
| | | | | | | | | | | | homolog; Method: conceptual |
| | | | | | | | | | | | translation supplied by author |
| 4489 | 4.66366942 | down | 0.0000034 | 0.0000712 | BU760898 | 370 | 10 | 30 | 33 | 6.3 | proteolysis and peptidolysis |
| 4490 | 4.662660945 | down | 0.000408859 | 0.00189949 | AW309958 | 675 | 97 | 147 | 65 | 8e−50 | defense response |
| 4491 | 4.6620224 | down | 0.00000319 | 0.0000683 | CK606628 | 641 | 45 | 124 | 36 | 7e−10 | MRNA from stress-induced gene (H4) |
| 4492 | 4.661477563 | down | 0.000000208 | 0.0000133 | CA936122 | 495 | 16 | 36 | 44 | 1.9 | signal transduction |
| 4493 | 4.659828736 | down | 0.000000474 | 0.0000216 | AW348132 | 721 | 139 | 238 | 58 | 2e−72 | lipid metabolism |
| 4494 | 4.657772577 | down | 0.00000691 | 0.000110275 | BM093848 | 229 | 7 | 17 | 41 | 3.2 | |
| 4495 | 4.650117188 | down | 0.00000123 | 0.0000372 | CD405056 | 664 | 34 | 49 | 69 | 6e−18 | G-protein coupled receptor protein |
| | | | | | | | | | | | signaling pathway |
| 4496 | 4.649748966 | down | 0.00000005 | 0.00000597 | BG046605 | 604 | 134 | 186 | 72 | 3e−75 | |
| 4497 | 4.645039735 | down | 0.00000316 | 0.000068 | BM885752 | 406 | 34 | 51 | 66 | 4e−12 | dUTP metabolism |
| 4498 | 4.632270751 | down | 0.00000025 | 0.0000146 | CA782920 | 698 | 32 | 83 | 38 | 5e−04 | |
| 4499 | 4.626353334 | down | 0.000416097 | 0.001923208 | BQ613105 | 536 | 42 | 141 | 29 | 5e−05 | protein amino acid phosphorylation |
| 4500 | 4.626295583 | down | 0.000000972 | 0.0000331 | Y10492.1 | | | | | | Putative cytochrome P450, clone CP5 |
| 4501 | 4.624486701 | down | 0.00000026 | 0.0000148 | AW306479 | 492 | 99 | 161 | 61 | 2e−46 | |
| 4502 | 4.615134857 | down | 0.000000124 | 0.00000982 | BQ298594 | 262 | 20 | 21 | 95 | 3e−05 | transport |
| 4503 | 4.610446556 | down | 0.004627708 | 0.010824365 | AW348397 | 790 | 110 | 212 | 51 | 1e−59 | lipoxygenase |
| 4504 | 4.608762262 | down | 0.000000208 | 0.0000133 | CD406580 | 489 | 31 | 51 | 60 | 4e−13 | |
| 4505 | 4.605938732 | down | 0.00000542 | 0.0000955 | BF071200 | 461 | 34 | 45 | 75 | 6e−14 | DNA replication initiation |
| 4506 | 4.600423693 | down | 0.0000991 | 0.000683525 | BQ611541 | 199 | 16 | 54 | 29 | 5.4 | sulfate assimilation |
| 4507 | 4.599415951 | down | 0.00000192 | 0.0000497 | BI969955 | 715 | 72 | 168 | 42 | 3e−30 | metabolism |
| 4508 | 4.59622488 | down | 0.000000622 | 0.0000253 | BQ474028 | 379 | 13 | 63 | 20 | 3.0 | |
| 4509 | 4.595004116 | down | 0.000000109 | 0.0000091 | CD401651 | 605 | 31 | 94 | 32 | 0.008 | |
| 4510 | 4.593304052 | down | 7.04E−08 | 0.00000729 | BG508624 | 364 | 15 | 50 | 30 | 0.92 | |
| 4511 | 4.589809664 | down | 8.46E−08 | 0.00000788 | AW348689 | 573 | 23 | 34 | 67 | 6e−07 | ciliary/flagellar motility |
| 4512 | 4.589593752 | down | 3.72E−08 | 0.00000511 | BE021574 | 401 | 65 | 132 | 49 | 4e−27 | response to oxidative stress |
| 4513 | 4.589484262 | down | 0.00000139 | 0.0000403 | CD411966 | 511 | 94 | 112 | 83 | 3e−51 | proteolysis and peptidolysis |
| 4514 | 4.589264026 | down | 0.000000498 | 0.0000222 | CF806940 | 714 | 62 | 140 | 44 | 3e−31 | transport |
| 4515 | 4.588954261 | down | 0.000000425 | 0.0000202 | BQ298358 | 413 | 29 | 53 | 54 | 2e−10 | regulation of transcription |
| 4516 | 4.58703624 | down | 0.00000444 | 0.0000843 | BM890917 | 525 | 51 | 170 | 30 | 4e−16 | hypersensitive response |
| 4517 | 4.586547625 | down | 0.000000509 | 0.0000225 | BI968013 | 644 | 44 | 61 | 72 | 7e−26 | G-protein coupled receptor protein |
| | | | | | | | | | | | signaling pathway |
| 4518 | 4.579897667 | down | 0.00000523 | 0.0000932 | CF805784 | 537 | 52 | 167 | 31 | 2e−18 | |
| 4519 | 4.577394828 | down | 0.00000249 | 0.0000587 | U20502.1 | | | | | | calnexin |
| 4520 | 4.576057793 | down | 0.000000847 | 0.0000305 | CD392205 | 510 | 56 | 97 | 57 | 2e−29 | carbohydrate metabolism |
| 4521 | 4.575426188 | down | 0.00000198 | 0.0000507 | CA782339 | 671 | 39 | 96 | 40 | 2e−07 | regulation of transcription |
| 4522 | 4.571061041 | down | 0.000000121 | 0.00000972 | CF805977 | 620 | 59 | 207 | 28 | 6e−06 | |
| 4523 | 4.570601208 | down | 0.0000184 | 0.000211968 | CD396215 | 671 | 98 | 133 | 73 | 1e−54 | isoprenoid biosynthesis |
| 4524 | 4.569723731 | down | 0.00000071 | 0.0000276 | AW277882 | 537 | 116 | 176 | 65 | 5e−61 | apoptosis |
| 4525 | 4.563185521 | down | 0.0000178 | 0.000207329 | AI988123 | 411 | 27 | 73 | 36 | 8e−09 | protein amino acid phosphorylation |
| 4526 | 4.562318827 | down | 0.000000218 | 0.0000137 | BM522568 | 388 | 27 | 27 | 100 | 8e−10 | microtubule-based movement |
| 4527 | 4.555951959 | down | 0.000000186 | 0.0000123 | BM188776 | 567 | 114 | 186 | 61 | 1e−61 | |
| 4528 | 4.550995704 | down | 0.0000281 | 0.000284288 | CD394600 | 673 | 92 | 124 | 74 | 4e−52 | proteolysis and peptidolysis |
| 4529 | 4.549012249 | down | 0.00000015 | 0.000011 | BQ741033 | 427 | 21 | 49 | 42 | 6e−06 | |
| 4530 | 4.545913232 | down | 0.000000815 | 0.0000298 | BG042982 | 707 | 138 | 237 | 58 | 1e−71 | electron transport |
| 4531 | 4.541492168 | down | 3.43E−08 | 0.00000493 | AI443042 | 532 | 104 | 128 | 81 | 4e−51 | Chalcone synthase [soybeans, mRNA, |
| | | | | | | | | | | | 1119 nt], flavonoid biosynthesis |
| 4532 | 4.539972631 | down | 0.00000749 | 0.000116042 | BI472156 | 395 | 36 | 59 | 61 | 1e−14 | proteolysis and peptidolysis |
| 4533 | 4.532877223 | down | 0.000000234 | 0.0000141 | AW310551 | 491 | 55 | 100 | 55 | 3e−24 | cytokinesis by cell plate formation |
| 4534 | 4.530171978 | down | 0.0000093 | 0.000132901 | BQ299492 | 549 | 158 | 183 | 86 | 2e−98 | |
| 4535 | 4.524762788 | down | 0.00000363 | 0.0000741 | BM527366 | 519 | 41 | 62 | 66 | 9e−23 | G-protein coupled receptor protein |
| | | | | | | | | | | | signaling pathway |
| 4536 | 4.522980503 | down | 0.000000197 | 0.0000128 | U36191.1 | | | | | | lipoxygenase |
| 4537 | 4.521269555 | down | 0.00000167 | 0.0000451 | CD395627 | 287 | 10 | 22 | 45 | 5.4 | transport |
| 4538 | 4.518932361 | down | 0.00000825 | 0.00012271 | CF808603 | 652 | 94 | 143 | 65 | 3e−55 | |
| 4639 | 4.516480565 | down | 0.0000167 | 0.00019915 | CD392652 | 381 | 12 | 33 | 36 | 4.0 | |
| 4540 | 4.508701869 | down | 0.0000384 | 0.000352678 | AI495284 | 441 | 38 | 56 | 67 | 7e−19 | epidermal cell differentiation |
| 4541 | 4.508417282 | down | 3.89E−08 | 0.00000518 | BG239729 | 471 | 33 | 79 | 41 | 3e−11 | electron transport |
| 4542 | 4.505628031 | down | 0.00000206 | 0.000052 | BG653752 | 421 | 12 | 21 | 57 | 3.9 | regulation of transcription |
| 4543 | 4.497163686 | down | 0.000408945 | 0.00189949 | CF808204 | 157 | 14 | 36 | 38 | 0.64 | |
| 4544 | 4.494886439 | down | 0.0000503 | 0.000424667 | AW705829 | | | | | | |
| 4545 | 4.492370676 | down | 0.000002 | 0.000051 | BI471357 | 329 | 7 | 17 | 41 | 3.3 | |
| 4546 | 4.48981502 | down | 8.68E−08 | 0.00000796 | BM522991 | 428 | 17 | 45 | 37 | 0.22 | |
| 4547 | 4.485501905 | down | 0.0000106 | 0.000146426 | CD395611 | 359 | 13 | 34 | 38 | 3.4 | G-protein coupled receptor protein |
| | | | | | | | | | | | signaling pathway |
| 4548 | 4.48537599 | down | 0.000000158 | 0.0000112 | BU763547 | 562 | 137 | 187 | 73 | 5e−72 | |
| 4549 | 4.483419287 | down | 0.0000103 | 0.000143546 | BM307710 | 192 | 14 | 40 | 35 | 4.2 | proteolysis and peptidolysis |
| 4550 | 4.482746269 | down | 0.0000683 | 0.000526087 | AW348930 | 747 | 91 | 197 | 46 | 1e−42 | metabolism |
| 4551 | 4.481796946 | down | 0.00000852 | 0.000125382 | AW310453 | 543 | 77 | 102 | 75 | 7e−40 | metabolism |
| 4552 | 4.479722639 | down | 0.000232301 | 0.001259047 | AW317178 | 654 | 111 | 174 | 63 | 2e−55 | |
| 4553 | 4.478815476 | down | 8.06E−08 | 0.00000772 | BE820487 | 612 | 66 | 103 | 64 | 3e−34 | |
| 4554 | 4.475748216 | down | 0.00000631 | 0.000104931 | CF809212 | 540 | 27 | 64 | 42 | 5e−05 | |
| 4555 | 4.475066863 | down | 0.0000161 | 0.000194761 | CF808027 | 533 | 127 | 179 | 70 | 6e−68 | |
| 4556 | 4.474027913 | down | 0.00000028 | 0.0000155 | CA800410 | 596 | 84 | 158 | 53 | 2e−47 | carbohydrate metabolism |
| 4557 | 4.470230678 | down | 0.000000901 | 0.0000318 | AI442332 | 362 | 50 | 110 | 45 | 9e−23 | phospholipid metabolism |
| 4558 | 4.468978086 | down | 0.000101171 | 0.000692862 | BQ080839 | 524 | 56 | 65 | 86 | 1e−23 | protein biosynthesis |
| 4559 | 4.466829207 | down | 0.0000268 | 0.00027559 | AW706162 | 192 | 20 | 56 | 35 | 6e−04 | |
| 4560 | 4.461806196 | down | 0.00000211 | 0.0000528 | BQ610568 | 555 | 111 | 162 | 68 | 3e−63 | metabolism |
| 4561 | 4.457413601 | down | 0.00000138 | 0.00004 | BQ612435 | 439 | 42 | 83 | 50 | 3e−21 | dicarboxylic acid transport |
| 4562 | 4.455831315 | down | 0.0000309 | 0.000304355 | AW759469 | 591 | 157 | 196 | 80 | 3e−90 | proteolysis and peptidolysis |
| 4563 | 4.453815119 | down | 0.00000286 | 0.0000637 | BG157500 | 640 | 77 | 155 | 49 | 2e−38 | |
| 4564 | 4.453647062 | down | 4.16E−08 | 0.00000536 | AI437572 | 432 | 78 | 135 | 57 | 7e−45 | chitin catabolism |
| 4565 | 4.451875735 | down | 0.000000446 | 0.0000208 | BI970672 | 780 | 44 | 91 | 48 | 7e−17 | |
| 4566 | 4.4492224 | down | 0.00000111 | 0.0000354 | BE661181 | 646 | 107 | 192 | 55 | 6e−52 | transport |
| 4567 | 4.448977817 | down | 0.0000417 | 0.000373852 | AW349732 | 769 | 105 | 145 | 72 | 1e−56 | |
| 4568 | 4.441883994 | down | 0.00000749 | 0.000116042 | AW100581 | 281 | 18 | 61 | 29 | 0.22 | |
| 4569 | 4.439282323 | down | 0.000000762 | 0.0000289 | CF808766 | 698 | 177 | 226 | 78 | e−108 | |
| 4570 | 4.435990839 | down | 0.00000728 | 0.000114115 | CD399897 | 678 | 44 | 55 | 80 | 5e−18 | aromatic compound metabolism |
| 4571 | 4.433307672 | down | 0.0000282 | 0.000285364 | BE023599 | 251 | 24 | 43 | 55 | 1e−09 | electron transport |
| 4572 | 4.432355759 | down | 0.000000563 | 0.000024 | CD397900 | 622 | 17 | 31 | 54 | 0.002 | |
| 4573 | 4.427983548 | down | 0.00000315 | 0.0000678 | AW101999 | 435 | 57 | 125 | 45 | 3e−21 | electron transport |
| 4574 | 4.426779536 | down | 0.00000503 | 0.0000912 | BG156321 | 513 | 52 | 110 | 47 | 8e−24 | proteolysis and peptidolysis |
| 4575 | 4.426451507 | down | 0.00000112 | 0.0000356 | BF069047 | 636 | 51 | 154 | 33 | 3e−19 | |
| 4576 | 4.423746226 | down | 0.00000886 | 0.000128818 | AW349057 | 661 | 50 | 93 | 53 | 1e−17 | Calnexin, sodium ion transport |
| 4577 | 4.413083231 | down | 0.00000437 | 0.0000834 | CD393521 | 540 | 36 | 100 | 36 | 1e−08 | |
| 4578 | 4.412767227 | down | 0.000229028 | 0.001244536 | BI700389 | 555 | 87 | 179 | 48 | 2e−43 | |
| 4579 | 4.411136531 | down | 0.0000907 | 0.000643608 | AW569146 | 436 | 14 | 35 | 40 | 1.9 | |
| 4580 | 4.409264711 | down | 0.00000033 | 0.0000172 | BE020759 | 392 | 105 | 128 | 82 | 2e−57 | |
| 4581 | 4.408348409 | down | 8.57E−08 | 0.00000795 | AW317183 | 628 | 73 | 130 | 56 | 6e−44 | |
| 4582 | 4.40565402 | down | 0.00000614 | 0.000102878 | AW186256 | 307 | 64 | 103 | 62 | 2e−31 | proteolysis and peptidolysis |
| 4583 | 4.405403354 | down | 0.00000218 | 0.0000538 | BI971888 | 539 | 95 | 114 | 83 | 1e−51 | dUTP metabolism |
| 4584 | 4.403807977 | down | 0.00000193 | 0.0000497 | BQ610840 | 514 | 85 | 110 | 77 | 2e−48 | |
| 4585 | 4.403491013 | down | 0.000102775 | 0.000700911 | CD409927 | 700 | 42 | 62 | 67 | 7e−23 | G-protein coupled receptor protein |
| | | | | | | | | | | | signaling pathway |
| 4586 | 4.402243961 | down | 0.0000222 | 0.000241835 | BU762688 | 570 | 143 | 189 | 75 | 5e−83 | protein amino acid phosphorylation |
| 4587 | 4.402165315 | down | 0.000797188 | 0.00308619 | BQ612650 | | | | | | |
| 4588 | 4.400670127 | down | 0.000250026 | 0.001327193 | BG651255 | 406 | 53 | 76 | 69 | 1e−25 | |
| 4589 | 4.389737105 | down | 0.000000387 | 0.000019 | AW349341 | 727 | 74 | 139 | 53 | 1e−36 | |
| 4590 | 4.385934492 | down | 0.001410096 | 0.004661161 | BM092298 | 354 | 28 | 60 | 46 | 3e−09 | |
| 4591 | 4.38552311 | down | 3.68E−08 | 0.00000508 | BQ611195 | 441 | 12 | 29 | 41 | 7.6 | electron transport |
| 4592 | 4.38388122 | down | 0.00000055 | 0.0000236 | BI970888 | 665 | 52 | 135 | 38 | 4e−14 | |
| 4593 | 4.380937009 | down | 0.00000084 | 0.0000304 | BQ452791 | 430 | 40 | 60 | 66 | 2e−16 | ciliary/flagellar motility |
| 4594 | 4.379979833 | down | 0.00000289 | 0.0000642 | BE822696 | 655 | 77 | 139 | 55 | 1e−39 | response to oxidative stress |
| 4595 | 4.379525194 | down | 6.39E−08 | 0.00000691 | BI701070 | 518 | 35 | 42 | 83 | 2e−15 | |
| 4596 | 4.376891722 | down | 0.0000293 | 0.000292965 | AW278168 | 426 | 31 | 40 | 77 | 1e−10 | |
| 4597 | 4.369972792 | down | 0.000000319 | 0.0000169 | BQ629187 | 447 | 18 | 55 | 32 | 0.013 | |
| 4598 | 4.369742578 | down | 7.66E−08 | 0.00000748 | BI969790 | 442 | 19 | 49 | 38 | 3e−04 | metabolism |
| 4599 | 4.36650419 | down | 0.00000274 | 0.000062 | AI496668 | 426 | 55 | 70 | 78 | 1e−25 | |
| 4600 | 4.361139948 | down | 0.00000522 | 0.0000932 | BM086044 | 570 | 144 | 189 | 76 | 7e−78 | |
| 4601 | 4.360262891 | down | 0.00000228 | 0.0000554 | BG725261 | 480 | 89 | 158 | 56 | 4e−51 | |
| 4602 | 4.358976904 | down | 7.01E−08 | 0.00000729 | CF809035 | 720 | 148 | 237 | 62 | 3e−83 | glutamate biosynthesis |
| 4603 | 4.348303387 | down | 0.00000035 | 0.0000178 | BG652851 | 409 | 22 | 24 | 91 | 7e−09 | |
| 4604 | 4.346456334 | down | 0.000245485 | 0.001311044 | BE658596 | 655 | 18 | 42 | 42 | 0.13 | G-protein coupled receptor protein |
| | | | | | | | | | | | signaling pathway |
| 4605 | 4.34476569 | down | 3.14E−08 | 0.0000047 | BU083789 | 449 | 54 | 152 | 35 | 2e−29 | |
| 4606 | 4.343885782 | down | 0.000000384 | 0.000019 | AW100853 | | | | | | |
| 4607 | 4.341019892 | down | 0.0000042 | 0.0000813 | BI320632 | 573 | 51 | 187 | 27 | 1e−13 | hypersensitive response |
| 4608 | 4.34000352 | down | 0.0000101 | 0.000141443 | AI856145 | 264 | 53 | 69 | 76 | 4e−26 | |
| 4609 | 4.319875429 | down | 0.000000429 | 0.0000203 | BM085930 | 584 | 58 | 159 | 36 | 5e−23 | |
| 4610 | 4.319078777 | down | 0.0000133 | 0.000170634 | BF071188 | 402 | 112 | 133 | 84 | 2e−64 | L-serine biosynthesis |
| 4611 | 4.317340485 | down | 0.000000169 | 0.0000117 | BM732633 | 551 | 14 | 51 | 27 | 3.1 | Hydroxyproline-rich glycoprotein (hrgp) |
| 4612 | 4.314161105 | down | 0.000430304 | 0.001971673 | BE610052 | 562 | 36 | 41 | 87 | 2e−14 | |
| 4613 | 4.312760341 | down | 9.72E−08 | 0.00000849 | BM731975 | 534 | 106 | 174 | 60 | 5e−64 | |
| 4614 | 4.309112489 | down | 0.000102787 | 0.000700911 | AW348556 | 737 | 98 | 107 | 91 | 5e−53 | |
| 4615 | 4.296662623 | down | 0.0000198 | 0.000222823 | CF808330 | 711 | 123 | 223 | 55 | 9e−66 | NAD(P)H dependent 6′-deoxychalcone |
| | | | | | | | | | | | synthase, potassium ion transport |
| 4616 | 4.295967767 | down | 0.000000722 | 0.0000279 | CA850597 | 315 | 21 | 28 | 75 | 7e−08 | carbon utilization |
| 4617 | 4.295560053 | down | 0.000000104 | 0.00000883 | CA820109 | 403 | 23 | 25 | 92 | 1e−08 | cell-cell signaling |
| 4618 | 4.295453813 | down | 0.002950213 | 0.007873514 | AW099627 | 512 | 62 | 87 | 71 | 2e−31 | proteolysis and peptidolysis |
| 4619 | 4.294152655 | down | 6.89E−08 | 0.00000729 | CD415065 | 535 | 71 | 133 | 53 | 1e−34 | |
| 4620 | 4.290879544 | down | 0.0000259 | 0.000269629 | AW310777 | 678 | 121 | 153 | 79 | 6e−67 | electron transport |
| 4621 | 4.289083877 | down | 0.00000829 | 0.000123258 | AW459462 | 587 | 105 | 201 | 52 | 2e−55 | |
| 4622 | 4.288401992 | down | 0.00000108 | 0.0000349 | CA800172 | 376 | 40 | 77 | 51 | 4e−15 | olfaction |
| 4623 | 4.288137635 | down | 0.00000145 | 0.0000412 | CD394504 | 679 | 60 | 65 | 92 | 7e−30 | microtubule-based movement |
| 4624 | 4.28700987 | down | 0.000000156 | 0.0000112 | BE822435 | 626 | 82 | 110 | 74 | 6e−49 | aspartic proteinase 4, proteolysis and |
| | | | | | | | | | | | peptidolysis |
| 4625 | 4.28367447 | down | 0.000000768 | 0.000029 | BM188434 | 569 | 117 | 181 | 64 | 5e−68 | protein amino acid phosphorylation |
| 4626 | 4.282040351 | down | 0.00000327 | 0.0000693 | BG507997 | 522 | 120 | 173 | 69 | 2e−67 | |
| 4627 | 4.280429245 | down | 0.00000226 | 0.0000554 | BF425779 | 415 | 79 | 133 | 59 | 4e−42 | electron transport |
| 4628 | 4.2798833 | down | 0.00000493 | 0.0000901 | BI967589 | 719 | 21 | 40 | 52 | 2e−05 | |
| 4629 | 4.278633251 | down | 0.00000134 | 0.0000391 | BM188587 | 553 | 67 | 138 | 48 | 1e−28 | |
| 4630 | 4.277996831 | down | 0.00000388 | 0.0000773 | BQ080717 | 552 | 147 | 184 | 79 | 6e−79 | transport |
| 4631 | 4.277771711 | down | 0.000000101 | 0.00000867 | CF807842 | 569 | 140 | 160 | 87 | 5e−89 | Cytosolic glutamine synthetase |
| | | | | | | | | | | | GSbeta 1 |
| 4632 | 4.273000183 | down | 0.00000771 | 0.000117799 | CF805971 | 672 | 26 | 64 | 40 | 5e−12 | |
| 4633 | 4.270101197 | down | 0.000000947 | 0.0000326 | AW570594 | 609 | 178 | 201 | 88 | 3e−99 | BiP isoform B |
| 4634 | 4.269173569 | down | 0.0000165 | 0.000197702 | CA936951 | 555 | 62 | 159 | 38 | 8e−28 | |
| 4635 | 4.267110229 | down | 5.15E−08 | 0.00000614 | BG508856 | 488 | 84 | 127 | 66 | 2e−43 | response to oxidative stress |
| 4636 | 4.22483774 | down | 0.000142053 | 0.000883962 | CD412960 | 675 | 76 | 138 | 55 | 4e−39 | |
| 4637 | 4.220668695 | down | 0.000000466 | 0.0000214 | CD392418 | 640 | 40 | 84 | 47 | 3e−12 | |
| 4638 | 4.21765485 | down | 0.000280777 | 0.00144559 | BI317229 | 230 | 9 | 16 | 56 | 1.4 | intracellular signaling cascade |
| 4639 | 4.215596516 | down | 0.0000153 | 0.000188348 | U26457.1 | | | | | | lipoxygenase |
| 4640 | 4.215581766 | down | 7.15E−08 | 0.00000732 | BG043694 | 533 | 41 | 76 | 53 | 2e−16 | |
| 4641 | 4.215502312 | down | 0.000000498 | 0.0000222 | AW350108 | 678 | 28 | 103 | 27 | 1.5 | G-protein coupled receptor protein |
| | | | | | | | | | | | signaling pathway |
| 4642 | 4.214851291 | down | 0.00000122 | 0.0000371 | BU544403 | 377 | 15 | 52 | 28 | 0.60 | |
| 4643 | 4.212570273 | down | 0.000000911 | 0.0000318 | CF921163 | 720 | 137 | 168 | 81 | 1e−81 | |
| 4644 | 4.209719636 | down | 0.000000464 | 0.0000214 | CF808030 | 519 | 88 | 150 | 58 | 6e−43 | |
| 4645 | 4.207303008 | down | 0.00000121 | 0.0000371 | BU765177 | 425 | 12 | 14 | 85 | 0.62 | |
| 4646 | 4.207132591 | down | 0.00000248 | 0.0000587 | CD409235 | 605 | 37 | 46 | 80 | 1e−15 | protein amino acid phosphorylation |
| 4647 | 4.205756201 | down | 0.000000258 | 0.0000148 | BQ628831 | 257 | 9 | 22 | 40 | 7.2 | |
| 4648 | 4.203485978 | down | 0.0000123 | 0.000161223 | CF808755 | 652 | 135 | 221 | 61 | 8e−70 | metabolism |
| 4649 | 4.202391935 | down | 0.0000035 | 0.0000725 | CF808362 | 481 | 129 | 153 | 84 | 1e−71 | |
| 4650 | 4.200107855 | down | 0.0000185 | 0.000212611 | CD414538 | 591 | 44 | 80 | 55 | 1e−23 | |
| 4651 | 4.197941613 | down | 0.0000141 | 0.000177449 | BG882829 | 471 | 78 | 100 | 78 | 3e−39 | |
| 4652 | 4.197934956 | down | 0.0000104 | 0.000144328 | BG650352 | 422 | 13 | 30 | 43 | 3.0 | protein amino acid phosphorylation |
| 4653 | 4.197301531 | down | 0.0000237 | 0.000253128 | BU550313 | 710 | 96 | 148 | 64 | 3e−51 | DNA replication initiation |
| 4654 | 4.195564901 | down | 0.00000176 | 0.0000466 | AW349565 | 697 | 106 | 173 | 61 | 7e−63 | protein amino acid phosphorylation |
| 4655 | 4.192126102 | down | 0.00000104 | 0.0000342 | CF807456 | 788 | 183 | 262 | 69 | e−108 | |
| 4656 | 4.191444131 | down | 0.002107172 | 0.006210716 | BE059056 | 260 | 12 | 29 | 41 | 0.38 | glycolysis |
| 4657 | 4.187353263 | down | 0.0000037 | 0.0000751 | BQ630178 | 606 | 77 | 130 | 59 | 9e−36 | response to oxidative stress |
| 4658 | 4.187007449 | down | 0.000000231 | 0.000014 | AW101089 | 205 | 10 | 24 | 41 | 9.2 | |
| 4659 | 4.185277436 | down | 0.00000471 | 0.0000873 | AI930627 | 491 | 87 | 163 | 53 | 1e−46 | |
| 4660 | 4.180432202 | down | 0.00000384 | 0.0000768 | BE555317 | 419 | 74 | 137 | 54 | 8e−35 | D-ribose metabolism |
| 4661 | 4.178182893 | down | 0.00000867 | 0.000126896 | CD404425 | 598 | 10 | 20 | 50 | 6.1 | |
| 4662 | 4.177961928 | down | 0.0000215 | 0.000235852 | CA852529 | 408 | 21 | 68 | 30 | 2.2 | protein amino acid phosphorylation |
| 4663 | 4.174784976 | down | 0.0000101 | 0.000141685 | AF118784.1 | | | | | | ribonucleotide reductase large subunit A |
| 4664 | 4.171583405 | down | 0.00000184 | 0.0000481 | AW309311 | 576 | 13 | 41 | 31 | 7.5 | |
| 4665 | 4.17029064 | down | 0.0000153 | 0.000188348 | AW349057 | 661 | 50 | 93 | 53 | 1e−17 | Calnexin, sodium ion transport |
| 4666 | 4.162930405 | down | 0.000000569 | 0.0000242 | BE800382 | 457 | 62 | 123 | 50 | 2e−27 | |
| 4667 | 4.161915905 | down | 0.00000325 | 0.0000691 | AI494661 | 412 | 63 | 83 | 75 | 3e−33 | electron transport |
| 4668 | 4.161607787 | down | 0.00000977 | 0.000137939 | BI468752 | 395 | 11 | 26 | 42 | 0.88 | amino acid transport |
| 4669 | 4.160902263 | down | 0.000000837 | 0.0000303 | BE022964 | 381 | 10 | 24 | 41 | 3.1 | glycolysis |
| 4670 | 4.158225581 | down | 0.00000274 | 0.000062 | CA785238 | 742 | 205 | 230 | 89 | e−114 | |
| 4671 | 4.155972957 | down | 4.58E−08 | 0.00000565 | AW306889 | 426 | 70 | 111 | 63 | 2e−36 | protein amino acid phosphorylation |
| 4672 | 4.154470033 | down | 0.00000022 | 0.0000137 | BI971185 | 551 | 30 | 55 | 54 | 5e−13 | |
| 4673 | 4.151950301 | down | 0.0000582 | 0.000470433 | BE023852 | 262 | 43 | 67 | 64 | 5e−20 | electron transport |
| 4674 | 4.15077707 | down | 0.000000892 | 0.0000315 | AW307000 | 353 | 11 | 34 | 32 | 5.6 | DNA replication |
| 4675 | 4.149362146 | down | 0.000106796 | 0.000719894 | BU546909 | 560 | 43 | 62 | 69 | 2e−20 | |
| 4676 | 4.14867913 | down | 0.000000685 | 0.0000271 | AW509163 | 484 | 39 | 44 | 88 | 2e−17 | DNA repair |
| 4677 | 4.146646515 | down | 0.00000205 | 0.0000517 | BU549354 | 562 | 96 | 128 | 75 | 2e−51 | actin cytoskeleton organization and |
| | | | | | | | | | | | biogenesis |
| 4678 | 4.140691644 | down | 0.00000921 | 0.000132376 | BM732969 | 489 | 44 | 59 | 74 | 2e−26 | |
| 4679 | 4.138683793 | down | 0.0000236 | 0.00025233 | BI317008 | 434 | 20 | 78 | 25 | 3.3 | RNA dependent DNA replication |
| 4680 | 4.137683796 | down | 0.000152807 | 0.000928695 | BE554841 | 639 | 144 | 213 | 67 | 2e−83 | transport |
| 4681 | 4.134319865 | down | 0.000112746 | 0.000748999 | BQ297116 | 352 | 11 | 28 | 39 | 4.2 | |
| 4682 | 4.134191389 | down | 0.000000993 | 0.0000336 | BU547841 | 658 | 50 | 161 | 31 | 1e−17 | |
| 4683 | 4.133172773 | down | 0.000009 | 0.000130265 | BU550583 | 650 | 54 | 126 | 42 | 3e−18 | |
| 4684 | 4.130753201 | down | 0.00000631 | 0.000104931 | BE824464 | 708 | 27 | 32 | 84 | 1e−10 | |
| 4685 | 4.130064468 | down | 0.000000286 | 0.0000157 | CD390492 | 642 | 78 | 105 | 74 | 2e−43 | |
| 4686 | 4.129144893 | down | 0.000000805 | 0.0000295 | CF807622 | 484 | 103 | 162 | 63 | 2e−57 | |
| 4687 | 4.128709804 | down | 0.000000243 | 0.0000144 | CA936869 | 540 | 62 | 125 | 49 | 2e−27 | protein amino acid phosphorylation |
| 4688 | 4.12703895 | down | 0.0000239 | 0.000254385 | BM143921 | 494 | 37 | 87 | 42 | 1e−13 | metabolism |
| 4689 | 4.12667247 | down | 0.0000266 | 0.000273782 | AI416624 | 338 | 71 | 111 | 63 | 3e−40 | |
| 4690 | 4.124957691 | down | 0.000000186 | 0.0000123 | BM188587 | 553 | 67 | 138 | 48 | 1e−28 | |
| 4691 | 4.124506856 | down | 0.000025 | 0.000262771 | AF276302.1 | | | | | | chalcone isomerase |
| 4692 | 4.12187993 | down | 0.000119631 | 0.000782439 | BQ453582 | 348 | 13 | 49 | 26 | 1.1 | cell elongation |
| 4693 | 4.120203717 | down | 0.000000145 | 0.0000108 | CD397394 | 620 | 102 | 134 | 76 | 1e−53 | response to ethylene stimulus |
| 4694 | 4.119330481 | down | 0.00000432 | 0.0000828 | AW309542 | 533 | 15 | 44 | 34 | 2.9 | |
| 4695 | 4.118131152 | down | 0.000000304 | 0.0000164 | CF806169 | 542 | 130 | 167 | 77 | 1e−72 | electron transport |
| 4696 | 4.11693926 | down | 0.00000678 | 0.000109098 | CF808455 | 320 | 17 | 22 | 77 | 3e−04 | amino acid transport |
| 4697 | 4.116892214 | down | 0.000000741 | 0.0000284 | CD408043 | 606 | 45 | 86 | 52 | 7e−18 | |
| 4698 | 4.11477758 | down | 0.00000111 | 0.0000354 | AI495867 | 354 | 85 | 118 | 72 | 8e−52 | |
| 4699 | 4.113401624 | down | 0.000000198 | 0.0000128 | AW433291 | 443 | 46 | 137 | 33 | 6e−18 | |
| 4700 | 4.112830856 | down | 0.000000483 | 0.0000219 | D78510.1 | | | | | | Beta-glucan-elicitor receptor |
| 4701 | 4.109511876 | down | 0.00000577 | 0.0000989 | CA937243 | 467 | 12 | 24 | 50 | 2.3 | regulation of transcription\, DNA- |
| | | | | | | | | | | | dependent |
| 4702 | 3.789829787 | down | 0.000948269 | 0.003516881 | BU765875 | 341 | 9 | 17 | 52 | 4.2 | protein amino acid phosphorylation |
| 4703 | 3.789789289 | down | 0.00000102 | 0.0000339 | CD410115 | 653 | 88 | 141 | 62 | 6e−41 | ciliary/flagellar motility |
| 4704 | 3.789548242 | down | 0.0000214 | 0.000235441 | AW620761 | 492 | 14 | 29 | 48 | 0.50 | |
| 4705 | 3.787838325 | down | 0.000000517 | 0.0000226 | AW830642 | 555 | 119 | 187 | 63 | 1e−65 | intracellular protein transport |
| 4706 | 3.787718786 | down | 0.000000716 | 0.0000278 | BE329760 | 686 | 60 | 91 | 65 | 5e−29 | electron transport |
| 4707 | 3.786949991 | down | 0.0000131 | 0.000169289 | BU765236 | 247 | 34 | 48 | 70 | 3e−13 | gravitropism |
| 4708 | 3.785885515 | down | 0.0000726 | 0.000548296 | BG507644 | 503 | 103 | 150 | 68 | 1e−59 | |
| 4709 | 3.782101727 | down | 0.0000329 | 0.00031728 | BU549811 | 454 | 12 | 25 | 48 | 6.1 | |
| 4710 | 3.781998371 | down | 0.00000173 | 0.0000462 | CF806478 | 510 | 51 | 127 | 40 | 7e−15 | ciliary/flagellar motility |
| 4711 | 3.778026659 | down | 0.000000419 | 0.00002 | CA936960 | 545 | 21 | 49 | 42 | 1e−05 | |
| 4712 | 3.775346332 | down | 0.000000676 | 0.0000269 | CD394504 | 679 | 60 | 65 | 92 | 7e−30 | microtubule-based movement |
| 4713 | 4.107985825 | down | 0.000000176 | 0.0000119 | CA784884 | 434 | 56 | 90 | 62 | 6e−29 | dicarboxylic acid transport |
| 4714 | 4.106503236 | down | 0.00000122 | 0.0000371 | BM108105 | 571 | 46 | 117 | 39 | 2e−16 | transport |
| 4715 | 4.104739309 | down | 0.00000156 | 0.0000433 | AW310201 | 539 | 13 | 60 | 21 | 0.47 | carbohydrate metabolism |
| 4716 | 4.10327617 | down | 0.00000423 | 0.0000817 | AW350419 | 718 | 69 | 93 | 74 | 5e−34 | D-ribose metabolism |
| 4717 | 4.101857384 | down | 0.000000361 | 0.0000182 | BG238117 | 556 | 16 | 50 | 32 | 3.1 | protein amino acid phosphorylation |
| 4718 | 4.101508847 | down | 0.000000706 | 0.0000276 | AW348585 | 733 | 64 | 140 | 45 | 1e−31 | electron transport |
| 4719 | 4.09853819 | down | 9.34E−08 | 0.00000834 | BQ473221 | 516 | 25 | 76 | 32 | 0.32 | electron transport |
| 4720 | 4.093919768 | down | 0.000000126 | 0.0000099 | BI942201 | 603 | 130 | 201 | 64 | 1e−68 | proline biosynthesis |
| 4721 | 4.093780984 | down | 0.000000787 | 0.0000292 | BI317761 | 474 | 57 | 113 | 50 | 2e−18 | |
| 4722 | 4.093150259 | down | 0.0000334 | 0.000321577 | BU546927 | 564 | 73 | 143 | 51 | 6e−28 | |
| 4723 | 4.092551732 | down | 0.00000665 | 0.000107858 | BQ454144 | 612 | 73 | 118 | 61 | 2e−37 | biological_process unknown |
| 4724 | 4.091062408 | down | 0.000356923 | 0.001718329 | BQ272719 | 575 | 33 | 83 | 39 | 1e−06 | regulation of abscisic acid mediated |
| | | | | | | | | | | | signaling |
| 4725 | 4.090903687 | down | 0.0000643 | 0.000504747 | BI968208 | 781 | 144 | 180 | 80 | 2e−78 | flavonoid biosynthesis |
| 4726 | 4.088970968 | down | 0.000000263 | 0.0000149 | CA853683 | 502 | 98 | 162 | 60 | 4e−54 | proteolysis and peptidolysis |
| 4727 | 4.087686406 | down | 0.0000275 | 0.000280621 | CD398481 | 479 | 32 | 55 | 58 | 1e−09 | |
| 4728 | 4.087217851 | down | 0.000000826 | 0.0000301 | CF806121 | 683 | 150 | 201 | 74 | 2e−76 | ATP/ADP exchange |
| 4729 | 4.081254123 | down | 0.000012 | 0.000158814 | BM890715 | 557 | 68 | 139 | 48 | 2e−27 | protein amino acid phosphorylation |
| 4730 | 4.079961248 | down | 0.00000121 | 0.0000371 | BU927557 | 444 | 73 | 95 | 76 | 3e−40 | metabolism |
| 4731 | 4.079092908 | down | 0.00000241 | 0.0000576 | U77678.1 | | | | | | asparagine synthetase 2 |
| 4732 | 4.077521469 | down | 0.00000409 | 0.0000799 | AW350271 | 628 | 94 | 117 | 80 | 4e−48 | |
| 4733 | 4.075918017 | down | 0.0000866 | 0.000622251 | BG239756 | 449 | 94 | 121 | 77 | 4e−54 | |
| 4734 | 4.07435042 | down | 0.000000289 | 0.0000158 | AW317183 | 628 | 73 | 130 | 56 | 6e−44 | |
| 4735 | 4.074225808 | down | 0.000026 | 0.00027023 | AW349057 | 661 | 50 | 93 | 53 | 1e−17 | calnexin, sodium ion transport |
| 4736 | 4.07284628 | down | 0.000000238 | 0.0000142 | CD418266 | 681 | 106 | 148 | 71 | 2e−60 | |
| 4737 | 4.072468929 | down | 0.000357977 | 0.001722181 | BI942310 | 358 | 10 | 21 | 47 | 2.5 | |
| 4738 | 4.07153469 | down | 0.0000328 | 0.000316495 | BU546272 | 621 | 39 | 62 | 62 | 2e−19 | |
| 4739 | 4.070347717 | down | 0.00000196 | 0.0000503 | AF118785.1 | | | | | | ribonucleotide reductase large subunit B |
| 4740 | 4.068245642 | down | 0.00000325 | 0.0000691 | AW348585 | 733 | 64 | 140 | 45 | 1e−31 | electron transport |
| 4741 | 4.067922449 | down | 8.72E−08 | 0.00000796 | AW349594 | 581 | 9 | 17 | 52 | 4.5 | electron transport |
| 4742 | 4.066448153 | down | 0.00000479 | 0.0000881 | BU765191 | 409 | 16 | 33 | 48 | 0.018 | electron transport |
| 4743 | 4.064586445 | down | 0.00000165 | 0.0000447 | BE023934 | 220 | 11 | 30 | 36 | 4.2 | protein amino acid phosphorylation |
| 4744 | 4.063009257 | down | 0.0000106 | 0.000146441 | CF806273 | 449 | 12 | 21 | 57 | 2.1 | |
| 4745 | 4.062651023 | down | 0.00000053 | 0.0000231 | AW704544 | 483 | 67 | 163 | 41 | 5e−32 | |
| 4746 | 4.060933016 | down | 0.000000425 | 0.0000202 | AI856228 | 523 | 87 | 141 | 61 | 1e−40 | ciliary/flagellar motility |
| 4747 | 4.060534958 | down | 0.00145191 | 0.004760162 | BU762610 | 359 | 35 | 49 | 71 | 7e−17 | threonine catabolism |
| 4748 | 4.059584794 | down | 0.00000504 | 0.0000914 | BM187663 | 531 | 25 | 92 | 27 | 0.20 | |
| 4749 | 4.058311858 | down | 0.000113007 | 0.000749934 | BI317410 | 467 | 113 | 169 | 66 | 2e−61 | |
| 4750 | 4.057531959 | down | 0.00000187 | 0.0000487 | BE658834 | 667 | 56 | 144 | 38 | 2e−23 | |
| 4751 | 4.055431986 | down | 0.0000645 | 0.000506076 | BM522903 | 405 | 18 | 24 | 75 | 0.001 | electron transport |
| 4752 | 4.053962753 | down | 0.000000795 | 0.0000294 | AI856228 | 523 | 87 | 141 | 61 | 1e−40 | ciliary/flagellar motility |
| 4753 | 4.0529337 | down | 0.0000305 | 0.000301686 | CD407199 | 574 | 14 | 42 | 33 | 2.0 | |
| 4754 | 4.052886761 | down | 0.000000845 | 0.0000305 | BU546989 | 579 | 83 | 125 | 66 | 1e−48 | |
| 4755 | 4.051236312 | down | 0.0000227 | 0.000245907 | BE824274 | 552 | 53 | 88 | 60 | 1e−24 | |
| 4756 | 4.050332058 | down | 0.00000105 | 0.0000342 | CF806672 | 645 | 49 | 88 | 55 | 5e−20 | G-protein coupled receptor protein |
| | | | | | | | | | | | signaling pathway |
| 4757 | 4.049334281 | down | 0.00000124 | 0.0000373 | BU548955 | 593 | 45 | 118 | 38 | 7e−14 | |
| 4758 | 4.048828483 | down | 6.14E−08 | 0.00000682 | BQ298558 | 362 | 11 | 48 | 22 | 6.0 | |
| 4759 | 4.048318778 | down | 0.000000232 | 0.000014 | BU551233 | 564 | 52 | 183 | 28 | 2e−13 | |
| 4760 | 4.047563375 | down | 0.000026 | 0.00027023 | BE556116 | | | | | | |
| 4761 | 4.042896347 | down | 0.000014 | 0.000176534 | BG156858 | 395 | 40 | 65 | 61 | 3e−18 | transport |
| 4762 | 4.042800311 | down | 0.000000895 | 0.0000316 | BQ611165 | 543 | 118 | 168 | 70 | 2e−60 | metabolism |
| 4763 | 4.042539036 | down | 0.00000169 | 0.0000454 | AW350559 | 491 | 46 | 58 | 79 | 7e−24 | electron transport |
| 4764 | 4.042416222 | down | 0.00000436 | 0.0000834 | BI317615 | 319 | 56 | 87 | 64 | 2e−27 | amino acid transport |
| 4765 | 4.041545318 | down | 0.00000469 | 0.0000871 | CD394414 | 476 | 20 | 31 | 64 | 6e−06 | carbohydrate metabolism |
| 4766 | 4.041180337 | down | 0.00000056 | 0.0000239 | CD394127 | 276 | 13 | 29 | 44 | 1.4 | |
| 4767 | 4.039571935 | down | 0.000327081 | 0.001614542 | BI970755 | 750 | 16 | 32 | 50 | 6.8 | amino acid metabolism |
| 4768 | 4.038612939 | down | 0.00000116 | 0.0000363 | CF807872 | 688 | 85 | 226 | 37 | 3e−25 | response to osmotic stress |
| 4769 | 4.036179626 | down | 0.00000012 | 0.00000969 | CF806081 | 657 | 137 | 200 | 68 | 3e−77 | galactose metabolism |
| 4770 | 4.032516869 | down | 0.000000414 | 0.0000199 | BQ453909 | 454 | 52 | 65 | 80 | 5e−26 | |
| 4771 | 4.031449 | down | 9.88E−08 | 0.00000857 | BI497646 | 392 | 81 | 129 | 62 | 6e−44 | protein amino acid phosphorylation |
| 4772 | 4.02746465 | down | 0.00000128 | 0.0000382 | CD395146 | 679 | 70 | 85 | 82 | 5e−36 | |
| 4773 | 4.026553608 | down | 0.000216197 | 0.001194131 | BI968600 | 790 | 79 | 125 | 63 | 2e−38 | |
| 4774 | 4.0251665 | down | 0.000532294 | 0.002304257 | AW309826 | 666 | 81 | 153 | 52 | 2e−46 | electron transport |
| 4775 | 4.023768295 | down | 0.0000279 | 0.000282692 | CF805870 | 678 | 192 | 224 | 85 | e−113 | Sucrose synthase (SS), response to |
| | | | | | | | | | | | cold |
| 4776 | 4.023215187 | down | 0.000000139 | 0.0000106 | BI973614 | 574 | 59 | 173 | 34 | 1e−24 | metabolism |
| 4777 | 4.021482583 | down | 0.000000909 | 0.0000318 | CF806595 | 695 | 90 | 223 | 40 | 1e−43 | metabolism |
| 4778 | 4.01961199 | down | 0.00000211 | 0.000053 | AB062754.1 | | | | | | ferritin |
| 4779 | 4.018429715 | down | 0.000000697 | 0.0000274 | CA802824 | 230 | 8 | 13 | 61 | 9.3 | proteolysis and peptidolysis |
| 4780 | 4.013997004 | down | 0.00000204 | 0.0000516 | AW832106 | 382 | 52 | 133 | 39 | 8e−14 | |
| 4781 | 4.013594314 | down | 0.000000191 | 0.0000126 | BI094851 | 583 | 30 | 56 | 53 | 6e−11 | |
| 4782 | 4.011558193 | down | 0.000000534 | 0.0000232 | BI700200 | 421 | 29 | 109 | 26 | 0.001 | |
| 4783 | 4.00786296 | down | 0.000000133 | 0.0000103 | CD398612 | 409 | 29 | 43 | 67 | 4e−12 | |
| 4784 | 4.004195476 | down | 0.00000245 | 0.0000581 | BE820602 | 338 | 21 | 29 | 72 | 6e−07 | response to light |
| 4785 | 4.001020139 | down | 0.00000223 | 0.0000547 | BF595987 | 377 | 11 | 28 | 39 | 3.0 | |
| 4786 | 3.998511898 | down | 0.000000105 | 0.00000891 | CD415333 | 393 | 11 | 34 | 32 | 3.4 | |
| 4787 | 3.998409635 | down | 0.000000737 | 0.0000283 | BM885206 | 569 | 156 | 189 | 82 | 2e−94 | carbohydrate metabolism |
| 4788 | 3.996749787 | down | 0.00000166 | 0.000045 | BI788127 | 384 | 14 | 43 | 32 | 2.4 | |
| 4789 | 3.996391152 | down | 0.00010781 | 0.000725175 | AW733248 | 397 | 37 | 59 | 62 | 2e−15 | |
| 4790 | 3.995745955 | down | 0.00000749 | 0.000116042 | AW279230 | 555 | 91 | 122 | 74 | 9e−53 | microtubule-based process |
| 4791 | 3.99457898 | down | 0.000000148 | 0.0000109 | CF806118 | 657 | 161 | 197 | 81 | 1e−89 | |
| 4792 | 3.994438424 | down | 0.0000053 | 0.0000942 | CD393504 | 505 | 25 | 50 | 50 | 4e−09 | tRNA aminoacylation for protein |
| | | | | | | | | | | | translation |
| 4793 | 3.992555789 | down | 0.0000312 | 0.000306205 | BU080998 | 619 | 106 | 195 | 54 | 7e−58 | electron transport |
| 4794 | 3.98968453 | down | 0.0000149 | 0.000185365 | BE657584 | 476 | 15 | 50 | 30 | 4.0 | |
| 4795 | 3.988271898 | down | 0.0000006 | 0.0000248 | BE440441 | 562 | 60 | 148 | 40 | 7e−27 | |
| 4796 | 3.987995025 | down | 0.0000164 | 0.000197137 | AI973546 | 435 | 13 | 42 | 30 | 1.1 | |
| 4797 | 3.987812651 | down | 0.0000353 | 0.000333289 | BQ610941 | 578 | 87 | 187 | 46 | 3e−43 | carbohydrate metabolism |
| 4798 | 3.986506812 | down | 0.0000426 | 0.000379406 | AW598676 | 578 | 132 | 193 | 68 | 2e−69 | oligopeptide transport |
| 4799 | 3.984622307 | down | 7.45E−08 | 0.00000744 | AW423572 | 294 | 14 | 36 | 38 | 1.5 | |
| 4800 | 3.983530297 | down | 0.00000123 | 0.0000372 | BE020061 | 376 | 65 | 119 | 54 | 1e−33 | |
| 4801 | 3.98098852 | down | 0.00031964 | 0.00158887 | BI945154 | 734 | 14 | 37 | 37 | 0.35 | Sali3-2 |
| 4802 | 3.978322354 | down | 0.000137072 | 0.000861749 | AW202394 | 411 | 14 | 53 | 26 | 0.34 | |
| 4803 | 3.975048587 | down | 0.0000344 | 0.000328015 | CD402671 | 596 | 75 | 105 | 71 | 5e−39 | |
| 4804 | 3.974438328 | down | 0.0000242 | 0.00025667 | BU547858 | 619 | 26 | 84 | 30 | 0.59 | |
| 4805 | 3.970025334 | down | 0.000206151 | 0.001152351 | BM954354 | 484 | 27 | 70 | 38 | 9e−05 | |
| 4806 | 3.96959192 | down | 0.000601554 | 0.002515211 | BQ612558 | 404 | 25 | 77 | 32 | 0.19 | cell death |
| 4807 | 3.96790982 | down | 0.000380364 | 0.001801889 | BQ453973 | 546 | 148 | 183 | 80 | 3e−84 | L-aspartate transport |
| 4808 | 3.965349495 | down | 0.000000112 | 0.0000092 | BG653402 | 244 | 69 | 78 | 88 | 8e−36 | |
| 4809 | 3.965257364 | down | 0.000000612 | 0.0000251 | CD391663 | 722 | 71 | 103 | 68 | 3e−34 | nucleosome assembly |
| 4810 | 3.962982968 | down | 0.000000433 | 0.0000205 | BQ612990 | 590 | 43 | 74 | 58 | 3e−14 | |
| 4811 | 3.962706814 | down | 0.000000391 | 0.0000191 | CD406079 | 652 | 117 | 146 | 80 | 2e−68 | brassinosteroid mediated signaling |
| 4812 | 3.961332633 | down | 0.00000141 | 0.0000406 | BG237767 | 409 | 11 | 26 | 42 | 1.3 | |
| 4813 | 3.953402739 | down | 0.00000314 | 0.0000678 | BE475550 | 593 | 53 | 95 | 55 | 1e−23 | |
| 4814 | 3.952140268 | down | 0.000000622 | 0.0000253 | U36191.1 | | | | | | lipoxygenase |
| 4815 | 3.946937792 | down | 0.0000311 | 0.000305536 | BG789619 | 336 | 10 | 32 | 31 | 5.5 | |
| 4816 | 3.946709023 | down | 0.0000468 | 0.000405665 | CF805620 | 538 | 127 | 169 | 75 | 2e−71 | |
| 4817 | 3.939361878 | down | 0.00000895 | 0.000129632 | CF806531 | 509 | 106 | 168 | 63 | 4e−56 | nucleotide-sugar metabolism |
| 4818 | 3.938080565 | down | 0.000094 | 0.000659533 | AW472501 | 466 | 24 | 47 | 51 | 9e−07 | carbohydrate metabolism |
| 4819 | 3.937846516 | down | 0.0000236 | 0.000252543 | AI794662 | 325 | 16 | 45 | 35 | 1.9 | |
| 4820 | 3.936639462 | down | 0.00000135 | 0.0000395 | BE611653 | 367 | 24 | 81 | 29 | 0.065 | |
| 4821 | 3.932903648 | down | 0.000000137 | 0.0000104 | AW310535 | 440 | 14 | 37 | 37 | 7.6 | |
| 4822 | 3.932579356 | down | 0.000095 | 0.000664724 | CA800540 | 629 | 31 | 35 | 88 | 9e−13 | regulation of transcription |
| 4823 | 3.928710463 | down | 0.0000106 | 0.000146441 | AI442864 | 500 | 30 | 67 | 44 | 3e−08 | |
| 4824 | 3.928320028 | down | 6.77E−08 | 0.00000721 | BI970055 | 771 | 47 | 117 | 40 | 4e−13 | |
| 4825 | 3.927724241 | down | 0.000000784 | 0.0000291 | AI443579 | 448 | 21 | 45 | 46 | 0.007 | tRNA processing |
| 4826 | 3.927373225 | down | 0.0000307 | 0.000302824 | AW395181 | 362 | 11 | 29 | 37 | 1.6 | |
| 4827 | 3.927141534 | down | 0.000000649 | 0.000026 | BQ453911 | 465 | 58 | 64 | 90 | 9e−31 | |
| 4828 | 3.92462364 | down | 0.000000745 | 0.0000284 | BI967748 | 762 | 116 | 141 | 82 | 2e−65 | glycolysis |
| 4829 | 3.923538115 | down | 0.00000534 | 0.0000946 | BG238497 | 572 | 109 | 188 | 57 | 1e−56 | proteolysis and peptidolysis |
| 4830 | 3.920734187 | down | 0.00000259 | 0.0000601 | BM568087 | 432 | 33 | 90 | 36 | 9e−08 | protein amino acid phosphorylation |
| 4831 | 3.92012567 | down | 0.000000196 | 0.0000128 | CF806406 | 669 | 123 | 205 | 60 | 9e−75 | |
| 4832 | 3.919148779 | down | 0.0000718 | 0.000544985 | CA800297 | 585 | 144 | 195 | 73 | 2e−85 | secondary cell wall biosynthesis (sensu |
| | | | | | | | | | | | Magnoliophyta) |
| 4833 | 3.91784539 | down | 0.000230738 | 0.001252197 | BQ627652 | 202 | 15 | 44 | 34 | 4.1 | |
| 4834 | 3.915774724 | down | 0.0000122 | 0.000161084 | CF808497 | 683 | 109 | 233 | 46 | 7e−49 | 2′-hydroxydihydrodaidzein reductase |
| 4835 | 3.91558805 | down | 0.000000192 | 0.0000127 | AW309536 | 631 | 66 | 128 | 51 | 3e−38 | carbohydrate metabolism |
| 4836 | 3.679873258 | down | 0.000000227 | 0.0000139 | CD418071 | 588 | 75 | 126 | 59 | 9e−38 | |
| 4837 | 3.679791694 | down | 0.00000726 | 0.000114049 | BQ628072 | 433 | 11 | 18 | 61 | 1.4 | electron transport |
| 4838 | 3.679781091 | down | 0.00000122 | 0.0000371 | BQ079445 | 572 | 82 | 117 | 70 | 4e−46 | electron transport |
| 4839 | 3.679680066 | down | 0.0000468 | 0.000405665 | AW597777 | 550 | 54 | 142 | 38 | 3e−23 | transport |
| 4840 | 3.679279547 | down | 0.000000498 | 0.0000222 | AW309759 | 663 | 30 | 48 | 62 | 3e−13 | carbohydrate metabolism |
| 4841 | 3.673683104 | down | 0.000336928 | 0.001647351 | AF243362.1 | | | | | | glutathione S-transferase GST 7 |
| 4842 | 3.673342858 | down | 0.00000146 | 0.0000415 | CF805726 | 569 | 63 | 83 | 75 | 2e−29 | dUTP metabolism |
| 4843 | 3.671920179 | down | 0.0000935 | 0.000657032 | CD396219 | 423 | 11 | 27 | 40 | 1.8 | |
| 4844 | 3.670183956 | down | 0.00000679 | 0.000109262 | BU549375 | 428 | 17 | 35 | 48 | 0.015 | electron transport |
| 4845 | 3.669780348 | down | 0.0000144 | 0.000180074 | AW309875 | 662 | 80 | 114 | 70 | 9e−43 | |
| 4846 | 3.667231006 | down | 0.00000638 | 0.000105523 | AW620511 | 464 | 116 | 152 | 76 | 2e−64 | transport |
| 4847 | 3.663449157 | down | 0.000000479 | 0.0000218 | BI967548 | 486 | 12 | 31 | 38 | 1.9 | hydroxyproline-rich glycoprotein |
| 4848 | 3.663372624 | down | 0.000585987 | 0.002473217 | CA819098 | 199 | 8 | 22 | 36 | 5.4 | |
| 4849 | 3.661679363 | down | 0.00000684 | 0.000109603 | BI321280 | 482 | 99 | 127 | 77 | 3e−50 | transport |
| 4850 | 3.661432798 | down | 0.0000147 | 0.000183603 | BI968010 | 427 | 47 | 64 | 73 | 6e−22 | regulation of transcription\, DNA- |
| | | | | | | | | | | | dependent |
| 4851 | 3.657691545 | down | 0.00000247 | 0.0000585 | BG510681 | 540 | 50 | 173 | 28 | 2e−11 | |
| 4852 | 3.654636648 | down | 0.0000351 | 0.000332586 | BU545084 | 592 | 73 | 171 | 42 | 7e−39 | |
| 4853 | 3.653438825 | down | 0.000000124 | 0.00000982 | BG839371 | 744 | 14 | 47 | 29 | 2.3 | |
| 4854 | 3.651659062 | down | 0.00000181 | 0.0000478 | CF805955 | 553 | 107 | 147 | 72 | 8e−60 | glycolysis |
| 4855 | 3.650983589 | down | 0.0000316 | 0.000309252 | CD392179 | 625 | 64 | 107 | 59 | 1e−31 | |
| 4856 | 3.650789863 | down | 0.0000106 | 0.000146298 | BE330476 | 552 | 78 | 101 | 77 | 2e−42 | transport |
| 4857 | 3.648627899 | down | 0.000000295 | 0.0000161 | BI973736 | 568 | 106 | 190 | 55 | 2e−56 | carbohydrate metabolism |
| 4858 | 3.643924927 | down | 0.000879428 | 0.003325504 | CD417852 | 622 | 36 | 81 | 44 | 3e−11 | Cyclin D3, regulation of cell cycle |
| 4859 | 3.642993048 | down | 0.00000479 | 0.0000881 | BI315773 | 368 | 11 | 25 | 44 | 4.8 | protein biosynthesis |
| 4860 | 3.642622334 | down | 0.000000329 | 0.0000172 | BE023715 | 365 | 14 | 48 | 29 | 4.7 | transport |
| 4861 | 3.642115592 | down | 0.0000213 | 0.000233968 | BI973916 | 429 | 64 | 141 | 45 | 6e−26 | G-protein coupled receptor protein |
| | | | | | | | | | | | signaling pathway |
| 4862 | 3.641870327 | down | 0.000000219 | 0.0000137 | BE823303 | 683 | 26 | 49 | 53 | 3e−09 | protein amino acid phosphorylation |
| 4863 | 3.641780151 | down | 0.0000352 | 0.000332586 | BI699570 | 580 | 78 | 99 | 78 | 6e−50 | carbohydrate biosynthesis |
| 4864 | 3.910408896 | down | 8.14E−08 | 0.00000775 | BQ612268 | 382 | 13 | 29 | 44 | 0.002 | dicarboxylic acid transport |
| 4865 | 3.907406338 | down | 0.00000229 | 0.0000555 | CD414593 | 574 | 57 | 81 | 70 | 7e−25 | |
| 4866 | 3.905078173 | down | 0.000013 | 0.00016783 | CF809161 | 548 | 45 | 68 | 66 | 5e−21 | regulation of transcription\, DNA- |
| | | | | | | | | | | | dependent |
| 4867 | 3.904299122 | down | 0.000170595 | 0.00100734 | CF807867 | 737 | 190 | 237 | 80 | e−117 | methionine metabolism |
| 4868 | 3.904100721 | down | 0.000000139 | 0.0000106 | BG156185 | 327 | 21 | 88 | 23 | 0.13 | |
| 4869 | 3.897471129 | down | 0.0000173 | 0.000203979 | AJ563365.1 | | | | | | monosaccharide transporter |
| 4870 | 3.89622502 | down | 0.0000014 | 0.0000404 | CD402460 | 702 | 32 | 119 | 26 | 0.001 | response to auxin stimulus |
| 4871 | 3.893454161 | down | 0.000254026 | 0.001343303 | BM884996 | 569 | 67 | 100 | 67 | 2e−31 | defense response |
| 4872 | 3.888331536 | down | 0.0000352 | 0.000333021 | BE057404 | 393 | 14 | 22 | 63 | 0.028 | regulation of transcription\, DNA- |
| | | | | | | | | | | | dependent |
| 4873 | 3.88784468 | down | 0.00000461 | 0.000086 | CA799411 | 560 | 68 | 87 | 78 | 1e−36 | regulation of transcription\, DNA- |
| | | | | | | | | | | | dependent |
| 4874 | 3.887278892 | down | 0.0000504 | 0.000424731 | AW307517 | 439 | 18 | 58 | 31 | 1.5 | proteolysis and peptidolysis |
| 4875 | 3.885450768 | down | 0.0000071 | 0.000112267 | BI315890 | 306 | 13 | 30 | 43 | 1.1 | |
| 4876 | 3.884419945 | down | 0.000000848 | 0.0000305 | BG046591 | 538 | 104 | 137 | 75 | 2e−57 | response to oxidative stress |
| 4877 | 3.883848311 | down | 0.000478565 | 0.002130516 | CD409660 | | | | | | |
| 4878 | 3.881220395 | down | 0.0000249 | 0.000262319 | BQ576507 | 400 | 37 | 84 | 44 | 2e−10 | regulation of transcription\, DNA- |
| | | | | | | | | | | | dependent |
| 4879 | 3.880470178 | down | 0.00000249 | 0.0000587 | CK605765 | 882 | 177 | 241 | 73 | e−106 | |
| 4880 | 3.875961143 | down | 0.0000265 | 0.000273368 | BG238502 | 656 | 69 | 217 | 31 | 1e−09 | transport |
| 4881 | 3.874913986 | down | 0.00000275 | 0.000062 | BQ630452 | 567 | 15 | 41 | 36 | 0.23 | |
| 4882 | 3.873761515 | down | 0.0000164 | 0.000196751 | BE347177 | 543 | 104 | 154 | 67 | 9e−53 | metabolism |
| 4883 | 3.870966503 | down | 0.0000054 | 0.0000952 | AW781253 | 423 | 16 | 50 | 32 | 0.16 | |
| 4884 | 3.870741296 | down | 0.000000624 | 0.0000253 | BG363116 | 562 | 39 | 74 | 52 | 2e−15 | carbohydrate metabolism |
| 4885 | 3.867019838 | down | 0.00000123 | 0.0000372 | BQ081493 | 420 | 31 | 69 | 44 | 5e−11 | |
| 4886 | 3.865564246 | down | 0.00000199 | 0.0000509 | BF067368 | 385 | 115 | 128 | 89 | 9e−61 | nucleotide-excision repair |
| 4887 | 3.865409776 | down | 0.00000591 | 0.000100315 | AB062754.1 | | | | | | ferritin |
| 4888 | 3.864225917 | down | 0.000000387 | 0.000019 | BI787495 | 593 | 121 | 147 | 82 | 2e−66 | water transport |
| 4889 | 3.862439368 | down | 0.00000553 | 0.0000966 | BU550178 | 702 | 122 | 155 | 78 | 3e−67 | proteolysis and peptidolysis |
| 4890 | 3.86103516 | down | 0.0000872 | 0.000625509 | CD401615 | 638 | 49 | 91 | 53 | 2e−16 | |
| 4891 | 3.85973505 | down | 0.00000012 | 0.00000969 | CF805767 | 542 | 46 | 74 | 62 | 7e−21 | |
| 4892 | 3.859173429 | down | 0.0000439 | 0.000387914 | BU548809 | 519 | 38 | 78 | 48 | 2e−11 | two-component signal transduction |
| | | | | | | | | | | | system (phosphorelay) |
| 4893 | 3.856712752 | down | 5.78E−08 | 0.0000066 | BG154867 | 488 | 114 | 158 | 72 | 4e−62 | |
| 4894 | 3.856613296 | down | 0.0000168 | 0.000199409 | BG045036 | 503 | 106 | 154 | 68 | 1e−61 | antibiotic biosynthesis |
| 4895 | 3.854211062 | down | 0.0000456 | 0.000398274 | BE610702 | 507 | 61 | 137 | 44 | 1e−22 | regulation of transcription |
| 4896 | 3.854004478 | down | 0.00000127 | 0.0000379 | BI426969 | 505 | 16 | 34 | 47 | 5.9 | |
| 4897 | 3.8539693 | down | 0.000419241 | 0.001934468 | BE023109 | 290 | 12 | 36 | 33 | 5.4 | |
| 4898 | 3.853333937 | down | 0.0000529 | 0.000439847 | CF806954 | 364 | 34 | 38 | 89 | 3e−15 | regulation of translation |
| 4899 | 3.852720101 | down | 0.000000708 | 0.0000276 | CD393284 | | | | | | |
| 4900 | 3.850473968 | down | 0.0000268 | 0.000275403 | BU550583 | 650 | 54 | 126 | 42 | 3e−18 | |
| 4901 | 3.85009557 | down | 0.0000129 | 0.000167666 | CA820031 | 583 | 66 | 238 | 27 | 6e−11 | regulation of transcription\, DNA- |
| | | | | | | | | | | | dependent |
| 4902 | 3.845059706 | down | 0.000000175 | 0.0000119 | BU763299 | 303 | 8 | 23 | 34 | 4.2 | transport |
| 4903 | 3.844064199 | down | 0.00000105 | 0.0000343 | AW102356 | 494 | 19 | 35 | 54 | 4e−04 | response to pathogenic bacteria |
| 4904 | 3.841745341 | down | 0.000000581 | 0.0000243 | AW396565 | 285 | 9 | 17 | 52 | 3.2 | protein amino acid phosphorylation |
| 4905 | 3.84142859 | down | 0.000000914 | 0.0000319 | BM568087 | 432 | 33 | 90 | 36 | 9e−08 | protein amino acid phosphorylation |
| 4906 | 3.840240335 | down | 0.00000369 | 0.0000751 | BG155028 | 549 | 126 | 166 | 75 | 3e−72 | |
| 4907 | 3.839583319 | down | 0.000000378 | 0.0000188 | CF805624 | 536 | 146 | 174 | 83 | 9e−79 | |
| 4908 | 3.837470153 | down | 0.0000386 | 0.000353073 | BE611786 | 288 | 13 | 30 | 43 | 1.1 | regulation of transcription\, DNA- |
| | | | | | | | | | | | dependent |
| 4909 | 3.837045979 | down | 0.000000296 | 0.0000161 | CD393841 | 679 | 35 | 92 | 38 | 8e−08 | protein biosynthesis |
| 4910 | 3.834787644 | down | 0.000000345 | 0.0000177 | CD406087 | 230 | 13 | 21 | 61 | 0.64 | |
| 4911 | 3.83423963 | down | 0.000000169 | 0.0000117 | BM092023 | 363 | 12 | 18 | 66 | 2.7 | metabolism |
| 4912 | 3.834147243 | down | 0.00000318 | 0.0000682 | BI967637 | 487 | 48 | 109 | 44 | 2e−13 | |
| 4913 | 3.834054081 | down | 0.000000512 | 0.0000226 | CD407619 | 682 | 28 | 30 | 93 | 6e−10 | ciliary/flagellar motility |
| 4914 | 3.833690159 | down | 0.000000736 | 0.0000283 | BF423817 | 405 | 15 | 42 | 35 | 6.1 | protein amino acid phosphorylation |
| 4915 | 3.833672011 | down | 0.000136673 | 0.000859956 | CD414022 | 621 | 50 | 103 | 48 | 5e−22 | proteolysis and peptidolysis |
| 4916 | 3.832919124 | down | 0.000000155 | 0.0000112 | CF806666 | 512 | 96 | 112 | 85 | 3e−52 | nucleotide-sugar metabolism |
| 4917 | 3.832295341 | down | 0.000064 | 0.000502427 | CA852105 | 692 | 41 | 77 | 53 | 1e−15 | proton transport |
| 4918 | 3.830632313 | down | 0.00000214 | 0.0000531 | BI971134 | 790 | 52 | 66 | 78 | 1e−23 | DNA repair |
| 4919 | 3.828803016 | down | 0.00000118 | 0.0000365 | AF243366.1 | | | | | | glutathione S-transferase GST 11 |
| 4920 | 3.828131207 | down | 0.000000249 | 0.0000146 | BU964633 | 500 | 70 | 105 | 66 | 2e−33 | |
| 4921 | 3.827974467 | down | 0.00000106 | 0.0000344 | CF806865 | 236 | 39 | 56 | 69 | 4e−17 | glycolysis |
| 4922 | 3.826936795 | down | 0.000000087 | 0.00000796 | BU551244 | 446 | 45 | 88 | 51 | 1e−21 | |
| 4923 | 3.824370481 | down | 0.000000164 | 0.0000115 | BI971324 | 750 | 86 | 167 | 51 | 4e−37 | metabolism |
| 4924 | 3.823279148 | down | 0.0000201 | 0.00022492 | AW757083 | 481 | 48 | 60 | 80 | 6e−23 | transport |
| 4925 | 3.821529886 | down | 0.00000105 | 0.0000343 | BU545459 | 495 | 79 | 126 | 62 | 5e−42 | carbohydrate metabolism |
| 4926 | 3.820398072 | down | 0.00000253 | 0.0000593 | AW309330 | 648 | 67 | 105 | 63 | 3e−30 | |
| 4927 | 3.820205193 | down | 0.0000078 | 0.000118926 | CF809212 | 540 | 27 | 64 | 42 | 5e−05 | |
| 4928 | 3.818319258 | down | 0.00000011 | 0.00000914 | BG237132 | 469 | 72 | 140 | 51 | 4e−27 | |
| 4929 | 3.816232689 | down | 0.00000156 | 0.0000434 | BU551370 | | | | | | |
| 4930 | 3.814877156 | down | 0.00000237 | 0.000057 | BU546096 | 720 | 106 | 198 | 53 | 1e−57 | protein amino acid phosphorylation |
| 4931 | 3.8143872 | down | 0.000000225 | 0.0000138 | CD411673 | 670 | 33 | 86 | 38 | 2e−05 | |
| 4932 | 3.814114717 | down | 8.95E−08 | 0.00000811 | BE330206 | 503 | 24 | 52 | 46 | 2e−04 | regulation of transcription\, DNA- |
| | | | | | | | | | | | dependent |
| 4933 | 3.813228993 | down | 0.000000514 | 0.0000226 | BU765504 | 597 | 74 | 216 | 34 | 4e−20 | |
| 4934 | 3.81098974 | down | 0.00000412 | 0.0000803 | BI702330 | 482 | 103 | 154 | 66 | 6e−47 | regulation of transcription |
| 4935 | 3.810622305 | down | 0.000105921 | 0.000715791 | AW278143 | | | | | | |
| 4936 | 3.810390219 | down | 0.00000445 | 0.0000844 | AW395454 | 513 | 130 | 177 | 73 | 1e−72 | electron transport |
| 4937 | 3.810306957 | down | 0.000000956 | 0.0000327 | CF809068 | 600 | 30 | 47 | 63 | 3e−14 | |
| 4938 | 3.808641624 | down | 0.00000173 | 0.0000462 | CD410010 | 330 | 11 | 20 | 55 | 1.1 | regulation of transcription |
| 4939 | 3.807207729 | down | 0.000000234 | 0.0000141 | CF808171 | 584 | 88 | 191 | 46 | 3e−45 | electron transport |
| 4940 | 3.80664526 | down | 0.000000617 | 0.0000252 | AW309927 | 666 | 85 | 149 | 57 | 2e−42 | |
| 4941 | 3.803403332 | down | 0.000000907 | 0.0000318 | CD413105 | 667 | 81 | 107 | 75 | 1e−45 | protein amino acid phosphorylation |
| 4942 | 3.802860692 | down | 0.00000176 | 0.0000468 | BG508595 | 469 | 33 | 54 | 61 | 5e−14 | protein amino acid phosphorylation |
| 4943 | 3.802633357 | down | 0.00000303 | 0.0000663 | BG650872 | 439 | 94 | 123 | 76 | 1e−54 | |
| 4944 | 3.801493029 | down | 0.0000013 | 0.0000384 | BE330057 | 622 | 38 | 98 | 38 | 2e−11 | regulation of transcription |
| 4945 | 3.800185146 | down | 0.000850723 | 0.003242715 | BI787790 | 476 | 48 | 66 | 72 | 2e−25 | |
| 4946 | 3.7995986 | down | 0.000491359 | 0.002171194 | BE059161 | 243 | 21 | 48 | 43 | 3.2 | phenylalanyl-tRNA aminoacylation |
| 4947 | 3.799350844 | down | 0.00000062 | 0.0000253 | BM887517 | 538 | 160 | 179 | 89 | 2e−95 | oligopeptide transport |
| 4948 | 3.798974865 | down | 0.00000235 | 0.0000566 | AW704362 | 563 | 141 | 193 | 73 | 4e−74 | |
| 4949 | 3.797457451 | down | 0.00000119 | 0.0000366 | AW309650 | 683 | 59 | 91 | 64 | 1e−26 | metabolism |
| 4950 | 3.797341464 | down | 0.00000159 | 0.0000439 | BE807486 | 366 | 11 | 32 | 34 | 4.7 | protein amino acid dephosphorylation |
| 4951 | 3.79499475 | down | 0.0000895 | 0.000636889 | BI967874 | 416 | 13 | 34 | 38 | 5.1 | |
| 4952 | 3.794251742 | down | 4.67E−08 | 0.00000572 | AW705849 | 538 | 43 | 100 | 43 | 1e−16 | |
| 4953 | 3.793622819 | down | 0.000000708 | 0.0000276 | BM568006 | 547 | 10 | 19 | 52 | 3.0 | Mo-molybdopterin cofactor biosynthesis |
| 4954 | 3.792278748 | down | 0.00000196 | 0.0000503 | CF807592 | 568 | 145 | 163 | 88 | 1e−77 | |
| 4955 | 3.791072957 | down | 0.00000267 | 0.0000612 | AW568561 | 475 | 35 | 46 | 76 | 2e−14 | |
| 4956 | 3.790981477 | down | 0.0000418 | 0.00037414 | AI522928 | 312 | 67 | 102 | 65 | 8e−36 | metabolism |
| 4957 | 3.79084961 | down | 0.0000109 | 0.000149019 | BE611653 | 367 | 24 | 81 | 29 | 0.065 | |
| 4958 | 3.790838341 | down | 0.000000203 | 0.000013 | BI425718 | 570 | 88 | 183 | 48 | 5e−46 | xyloglucan biosynthesis |
| 4959 | 3.774794958 | down | 0.00000242 | 0.0000577 | AF249318.1 | | | | | | Pti1 kinase-like protein |
| 4960 | 3.77449992 | down | 0.00000189 | 0.0000492 | CD401867 | 633 | 36 | 68 | 52 | 6e−09 | |
| 4961 | 3.774091953 | down | 0.000000719 | 0.0000279 | BI972227 | 365 | 19 | 25 | 76 | 2e−05 | proteolysis and peptidolysis |
| 4962 | 3.772661584 | down | 0.0000026 | 0.0000601 | CF807216 | 125 | 35 | 41 | 85 | 6e−15 | ATP/ADP exchange |
| 4963 | 3.771550954 | down | 0.000000439 | 0.0000206 | CD394479 | 609 | 60 | 73 | 82 | 2e−31 | |
| 4964 | 3.771237354 | down | 0.000000322 | 0.000017 | AW185294 | 551 | 63 | 77 | 81 | 3e−34 | |
| 4965 | 3.771134465 | down | 0.00000176 | 0.0000466 | AW394575 | 403 | 19 | 56 | 33 | 2.7 | |
| 4966 | 3.770404493 | down | 0.00000336 | 0.0000707 | BE820853 | 595 | 61 | 94 | 64 | 2e−31 | proteolysis and peptidolysis |
| 4967 | 3.769712789 | down | 0.00041306 | 0.001913075 | AW306682 | 409 | 13 | 27 | 48 | 0.33 | |
| 4968 | 3.768522338 | down | 0.0000389 | 0.000355131 | BE611219 | 506 | 102 | 127 | 80 | 8e−55 | transport |
| 4969 | 3.766561909 | down | 0.000206057 | 0.001152012 | CF808262 | 562 | 59 | 108 | 54 | 6e−33 | electron transport |
| 4970 | 3.766532879 | down | 0.000246784 | 0.00131601 | CF806688 | 716 | 185 | 217 | 85 | e−109 | |
| 4971 | 3.76537359 | down | 0.000000482 | 0.0000219 | BG044912 | 493 | 78 | 87 | 89 | 2e−42 | amino acid transport |
| 4972 | 3.764526167 | down | 0.000047 | 0.000406543 | BE820819 | 203 | 15 | 29 | 51 | 3.2 | regulation of transcription\, DNA- |
| | | | | | | | | | | | dependent |
| 4973 | 3.764462103 | down | 7.32E−08 | 0.00000738 | BE058555 | 465 | 98 | 133 | 73 | 2e−50 | transport |
| 4974 | 3.763772772 | down | 0.000000577 | 0.0000243 | CA935684 | 426 | 84 | 138 | 60 | 1e−45 | |
| 4975 | 3.763068964 | down | 0.000000879 | 0.0000311 | AW233808 | 473 | 131 | 155 | 84 | 1e−77 | |
| 4976 | 3.763059356 | down | 0.0000489 | 0.000416402 | AW349132 | 689 | 102 | 191 | 53 | 6e−58 | |
| 4977 | 3.761975527 | down | 0.000340366 | 0.001658815 | BM528748 | 568 | 170 | 189 | 89 | 4e−98 | |
| 4978 | 3.761924104 | down | 0.000138414 | 0.000868008 | BM524095 | 536 | 69 | 199 | 34 | 5e−17 | |
| 4979 | 3.75947416 | down | 0.0000658 | 0.000512484 | BU549884 | 585 | 40 | 62 | 64 | 1e−16 | carbohydrate metabolism |
| 4980 | 3.757183684 | down | 0.00000353 | 0.0000729 | AW317183 | 628 | 73 | 130 | 56 | 6e−44 | |
| 4981 | 3.755554947 | down | 0.000139793 | 0.000873013 | AW831290 | 433 | 69 | 142 | 48 | 4e−34 | electron transport |
| 4982 | 3.754419283 | down | 0.000000583 | 0.0000244 | BQ612383 | 437 | 16 | 43 | 37 | 0.52 | |
| 4983 | 3.754382778 | down | 0.000151248 | 0.000921618 | CF806507 | 617 | 144 | 204 | 70 | 1e−81 | cell elongation |
| 4984 | 3.753775558 | down | 0.0000683 | 0.00052619 | CF806335 | 674 | 106 | 191 | 55 | 4e−47 | |
| 4985 | 3.751719981 | down | 0.0000934 | 0.000656461 | BI970927 | 574 | 10 | 25 | 40 | 2.6 | cellulose biosynthesis |
| 4986 | 3.75004537 | down | 0.0000219 | 0.000239247 | CA819837 | 220 | 16 | 58 | 27 | 0.020 | |
| 4987 | 3.748591064 | down | 0.000000183 | 0.0000123 | CA850597 | 315 | 21 | 28 | 75 | 7e−08 | carbon utilization |
| 4988 | 3.746078924 | down | 0.0000265 | 0.000273713 | BE821285 | 459 | 41 | 83 | 49 | 8e−22 | |
| 4989 | 3.746030172 | down | 0.0000173 | 0.000204023 | BM178135 | 373 | 13 | 23 | 56 | 1.00 | |
| 4990 | 3.744840129 | down | 0.00000123 | 0.0000372 | BM177226 | 427 | 21 | 59 | 35 | 0.13 | |
| 4991 | 3.744805989 | down | 0.00000718 | 0.000113104 | CD486940 | 579 | 26 | 30 | 86 | 1e−08 | ciliary/flagellar motility |
| 4992 | 3.744526659 | down | 0.00000115 | 0.0000361 | BE658864 | 219 | 17 | 35 | 48 | 2.5 | |
| 4993 | 3.743553566 | down | 0.0000102 | 0.000142234 | CA819837 | 220 | 16 | 58 | 27 | 0.020 | |
| 4994 | 3.733051314 | down | 0.000000108 | 0.00000905 | CD400862 | 698 | 137 | 157 | 87 | 3e−75 | glycine metabolism |
| 4995 | 3.733048153 | down | 0.00000478 | 0.0000881 | CF806066 | 656 | 161 | 216 | 74 | 7e−99 | cell elongation |
| 4996 | 3.732790019 | down | 0.00000532 | 0.0000943 | CD412146 | 648 | 62 | 133 | 46 | 3e−27 | |
| 4997 | 3.731642022 | down | 8.41E−08 | 0.00000785 | BU547639 | 354 | 15 | 43 | 34 | 4.3 | |
| 4998 | 3.730365262 | down | 0.0000035 | 0.0000725 | AI988121 | 115 | 11 | 30 | 36 | 2.5 | cell growth and/or maintenance |
| 4999 | 3.729619777 | down | 0.0000017 | 0.0000457 | BU091001 | 470 | 12 | 31 | 38 | 3.0 | protein complex assembly |
| 5000 | 3.727250848 | down | 0.000000938 | 0.0000325 | BG042570 | 633 | 70 | 133 | 52 | 7e−37 | |
| 5001 | 3.725606764 | down | 0.00000063 | 0.0000255 | BQ630533 | 602 | 143 | 195 | 73 | 7e−78 | |
| 5002 | 3.724935402 | down | 0.0000315 | 0.000308407 | BF067565 | 448 | 49 | 88 | 55 | 4e−21 | cell adhesion |
| 5003 | 3.724565218 | down | 0.000000391 | 0.0000191 | BI701413 | 568 | 41 | 171 | 23 | 4e−09 | metabolism |
| 5004 | 3.721956012 | down | 0.000133048 | 0.00084501 | CD407998 | 643 | 62 | 148 | 41 | 9e−27 | proteolysis and peptidolysis |
| 5005 | 3.721242425 | down | 0.00000046 | 0.0000213 | BM177977 | 562 | 56 | 118 | 47 | 2e−22 | response to oxidative stress |
| 5006 | 3.721236986 | down | 0.00000222 | 0.0000545 | AW186182 | 521 | 132 | 175 | 75 | 1e−74 | |
| 5007 | 3.719960891 | down | 0.000115174 | 0.000760637 | AI856188 | 548 | 11 | 27 | 40 | 5.2 | |
| 5008 | 3.719533525 | down | 0.000068 | 0.000524508 | BU549826 | 644 | 100 | 160 | 62 | 4e−53 | carbohydrate metabolism |
| 5009 | 3.716735384 | down | 0.000000155 | 0.0000112 | CA852815 | 396 | 11 | 19 | 57 | 1.2 | selenocysteine incorporation |
| 5010 | 3.716352674 | down | 0.00000433 | 0.000083 | AW311543 | 155 | 14 | 31 | 45 | 9.2 | |
| 5011 | 3.716033129 | down | 0.00000278 | 0.0000626 | BU550576 | 607 | 87 | 106 | 82 | 1e−49 | |
| 5012 | 3.713818202 | down | 0.000000563 | 0.000024 | BM954521 | 319 | 9 | 13 | 69 | 1.1 | protein amino acid phosphorylation |
| 5013 | 3.713588468 | down | 0.00000518 | 0.0000929 | BE823308 | 661 | 101 | 133 | 75 | 3e−53 | protein amino acid phosphorylation |
| 5014 | 3.712438357 | down | 0.00000164 | 0.0000446 | CA799668 | 720 | 131 | 232 | 56 | 1e−73 | |
| 5015 | 3.710469056 | down | 0.0000019 | 0.0000493 | CA803057 | 346 | 37 | 41 | 90 | 3e−15 | |
| 5016 | 3.706429652 | down | 0.0000244 | 0.000258214 | AW598203 | 480 | 105 | 157 | 66 | 1e−54 | |
| 5017 | 3.706232971 | down | 0.0000102 | 0.000142275 | BI970991 | 597 | 74 | 95 | 77 | 4e−39 | regulation of translation |
| 5018 | 3.705898216 | down | 0.00000451 | 0.000085 | CF809212 | 540 | 27 | 64 | 42 | 5e−05 | |
| 5019 | 3.705377457 | down | 0.0000114 | 0.000153343 | BU545195 | 522 | 27 | 44 | 61 | 4e−09 | carbohydrate metabolism |
| 5020 | 3.703980614 | down | 0.000043 | 0.000382673 | BI702152 | 362 | 32 | 49 | 65 | 2e−14 | |
| 5021 | 3.703929171 | down | 0.0000004 | 0.0000194 | BM307957 | 374 | 9 | 24 | 37 | 4.9 | electron transport |
| 5022 | 3.703431864 | down | 0.00000246 | 0.0000582 | BE022964 | 381 | 10 | 24 | 41 | 3.1 | glycolysis |
| 5023 | 3.702771225 | down | 0.000109445 | 0.000732766 | AI460729 | 402 | 49 | 89 | 55 | 4e−25 | proteolysis and peptidolysis |
| 5024 | 3.701476101 | down | 8.58E−08 | 0.00000795 | BG726403 | 320 | 15 | 35 | 42 | 0.83 | intracellular signaling cascade |
| 5025 | 3.700959325 | down | 0.000000197 | 0.0000128 | D13998.1 | | | | | | phosphoenolpyruvate carboxylase |
| 5026 | 3.699642983 | down | 0.00176757 | 0.005486497 | AW350194 | 786 | 108 | 190 | 56 | 9e−59 | |
| 5027 | 3.699187304 | down | 0.000000906 | 0.0000318 | CF808183 | 726 | 189 | 241 | 78 | e−112 | terpenoid biosynthesis |
| 5028 | 3.698637002 | down | 0.00000481 | 0.0000885 | BQ610340 | 381 | 45 | 66 | 68 | 2e−20 | DNA methylation |
| 5029 | 3.698152133 | down | 0.00000591 | 0.000100315 | BE821548 | 247 | 10 | 20 | 50 | 1.9 | |
| 5030 | 3.697240735 | down | 0.0000353 | 0.000333289 | BI094938 | 412 | 34 | 36 | 94 | 1e−14 | transport |
| 5031 | 3.696705713 | down | 0.00000592 | 0.000100437 | BI972404 | 525 | 40 | 120 | 33 | 3e−09 | |
| 5032 | 3.694955113 | down | 0.00000235 | 0.0000566 | AW460084 | 546 | 128 | 174 | 73 | 3e−71 | lipid biosynthesis |
| 5033 | 3.694890617 | down | 0.0000063 | 0.000104925 | BQ611020 | 370 | 10 | 17 | 58 | 1.3 | regulation of transcription\, DNA- |
| | | | | | | | | | | | dependent |
| 5034 | 3.694610924 | down | 0.0000103 | 0.000143348 | CF806381 | 690 | 186 | 211 | 88 | e−103 | |
| 5035 | 3.692445973 | down | 0.00000204 | 0.0000517 | BE823578 | 711 | 23 | 69 | 33 | 0.087 | |
| 5036 | 3.690851372 | down | 0.000000764 | 0.0000289 | CD398490 | 704 | 20 | 60 | 33 | 0.010 | |
| 5037 | 3.690057598 | down | 0.00000515 | 0.0000928 | BE473777 | 329 | 60 | 109 | 55 | 2e−28 | transport |
| 5038 | 3.688773198 | down | 0.00000034 | 0.0000175 | BG238158 | 550 | 43 | 96 | 44 | 5e−16 | response to reactive oxygen species |
| 5039 | 3.688480898 | down | 0.0000363 | 0.00033972 | CF808802 | 760 | 104 | 213 | 48 | 2e−39 | |
| 5040 | 3.684810824 | down | 0.0000384 | 0.000352676 | CD414923 | 462 | 62 | 100 | 62 | 4e−35 | electron transport |
| 5041 | 3.683895186 | down | 0.0000702 | 0.000536435 | BI471586 | 560 | 20 | 82 | 24 | 1.9 | |
| 5042 | 3.683858523 | down | 0.00000169 | 0.0000456 | BG404966 | 474 | 64 | 88 | 72 | 3e−29 | glycolysis |
| 5043 | 3.683725063 | down | 0.000000307 | 0.0000165 | AW309763 | 600 | 74 | 106 | 69 | 8e−37 | |
| 5044 | 3.683702707 | down | 0.0000011 | 0.0000351 | BM891946 | 563 | 70 | 145 | 48 | 3e−28 | proton transport |
| 5045 | 3.683172604 | down | 0.00000167 | 0.000045 | CD405317 | 387 | 11 | 27 | 40 | 4.2 | |
| 5046 | 3.683021432 | down | 0.000000136 | 0.0000104 | CF809046 | 548 | 132 | 164 | 60 | 4e−69 | gravitropism |
| 5047 | 3.681694577 | down | 0.000002 | 0.000051 | BE440328 | 569 | 103 | 151 | 68 | 8e−61 | |
| 5048 | 3.640927437 | down | 0.000000272 | 0.0000151 | CF807592 | 568 | 145 | 163 | 88 | 1e−77 | |
| 5049 | 3.640590227 | down | 0.00000058 | 0.0000243 | BQ253916 | 575 | 124 | 181 | 68 | 1e−65 | |
| 5050 | 3.639972958 | down | 0.0000307 | 0.000302735 | BE659875 | 762 | 118 | 231 | 51 | 2e−61 | carbohydrate metabolism |
| 5051 | 3.639434551 | down | 0.000000222 | 0.0000137 | S46989.1 | | | | | | Chalcone synthase [soybeans, mRNA, |
| | | | | | | | | | | | 1119 nt] |
| 5052 | 3.480358977 | down | 0.0000198 | 0.000222658 | CK606595 | 273 | 23 | 32 | 71 | 1e−08 | |
| 5053 | 3479943972 | down | 0.000000751 | 0.0000286 | CD414945 | 716 | 48 | 90 | 53 | 3e−22 | chromatin assembly/disassembly |
| 5054 | 3.479043717 | down | 0.000003 | 0.0000659 | BG653261 | 405 | 40 | 98 | 40 | 4e−12 | |
| 5055 | 3.477776085 | down | 0.00000382 | 0.0000765 | BU762209 | 548 | 32 | 119 | 26 | 0.009 | |
| 5056 | 3.47681705 | down | 0.0000135 | 0.000172562 | AI901151 | 555 | 127 | 142 | 89 | 3e−71 | small GTPase mediated signal |
| | | | | | | | | | | | transduction |
| 5057 | 3.475431598 | down | 0.000143874 | 0.000890955 | BU550673 | 534 | 29 | 101 | 28 | 3e−04 | response to virus |
| 5058 | 3.472960022 | down | 0.00000699 | 0.000111171 | BE820706 | 207 | 12 | 19 | 63 | 3.3 | transport |
| 5059 | 3.471176755 | down | 0.00000753 | 0.00011626 | BE659785 | 694 | 56 | 112 | 50 | 2e−24 | |
| 5060 | 3.469734741 | down | 0.00000193 | 0.0000497 | BU964415 | 583 | 121 | 198 | 61 | 3e−69 | proteolysis and peptidolysis |
| 5061 | 3.468104503 | down | 0.00000702 | 0.000111293 | CD396836 | 562 | 32 | 66 | 48 | 1e−05 | |
| 5062 | 3.467790651 | down | 0.0000164 | 0.000197137 | CD407159 | 587 | 67 | 94 | 71 | 4e−35 | transport |
| 5063 | 3.466348792 | down | 0.00000626 | 0.000104541 | BI970653 | 766 | 86 | 121 | 71 | 1e−51 | carbohydrate metabolism |
| 5064 | 3.464607176 | down | 0.0000906 | 0.000643099 | CD401803 | 499 | 28 | 46 | 60 | 2e−09 | |
| 5065 | 3.463117187 | down | 0.00000272 | 0.0000619 | AI522941 | 715 | 155 | 207 | 74 | 3e−99 | carbohydrate metabolism |
| 5066 | 3.462941249 | down | 0.00000539 | 0.0000951 | BE823570 | 666 | 32 | 98 | 32 | 1e−04 | |
| 5067 | 3.462099298 | down | 0.000000585 | 0.0000244 | BG046811 | 654 | 53 | 125 | 42 | 4e−22 | |
| 5068 | 3.461829511 | down | 0.0000168 | 0.000199797 | BE190451 | | | | | | |
| 5069 | 3.461772648 | down | 0.00000025 | 0.0000146 | BG508974 | 349 | 12 | 34 | 35 | 1.9 | |
| 5070 | 3.461310393 | down | 0.000027 | 0.000277134 | CF923111 | 468 | 33 | 70 | 47 | 3e−13 | |
| 5071 | 3.459255155 | down | 0.000312251 | 0.001563083 | CF806688 | 716 | 185 | 217 | 85 | e−109 | |
| 5072 | 3.459186725 | down | 0.00000373 | 0.0000754 | CA938627 | | | | | | |
| 5073 | 3.458902451 | down | 0.00000836 | 0.000123987 | BQ629239 | 521 | 48 | 73 | 65 | 8e−22 | |
| 5074 | 3.454610776 | down | 0.0000132 | 0.000170273 | AW349728 | 661 | 129 | 173 | 74 | 4e−70 | chloride transport |
| 5075 | 3.45443312 | down | 0.000359003 | 0.001725541 | BF067671 | 596 | 145 | 198 | 73 | 3e−78 | proteolysis and peptidolysis |
| 5076 | 3.452883184 | down | 0.000000324 | 0.000017 | BI972157 | 361 | 57 | 81 | 70 | 6e−31 | transport |
| 5077 | 3.412838003 | down | 0.0000224 | 0.000242928 | AW307241 | 609 | 54 | 120 | 45 | 4e−16 | |
| 5078 | 3.412436875 | down | 0.0000145 | 0.000181227 | BI971321 | 800 | 111 | 137 | 81 | 2e−63 | spermidine biosynthesis |
| 5079 | 3.41176228 | down | 0.00000268 | 0.0000614 | AW310958 | 377 | 16 | 43 | 37 | 3.0 | |
| 5080 | 3.409820265 | down | 0.00000832 | 0.000123535 | BM522997 | 406 | 38 | 53 | 71 | 9e−17 | DNA replication initiation |
| 5081 | 3.409484477 | down | 0.0000673 | 0.000520787 | BM522580 | 542 | 60 | 98 | 61 | 7e−32 | |
| 5082 | 3.406961465 | down | 0.00000738 | 0.000115167 | BG041500 | 307 | 71 | 90 | 78 | 5e−35 | transport |
| 5083 | 3.406652194 | down | 0.00000467 | 0.0000869 | AW597363 | 423 | 14 | 35 | 40 | 1.8 | apoptosis |
| 5084 | 3.405222399 | down | 0.00000649 | 0.000106296 | AW351068 | 579 | 35 | 59 | 59 | 5e−14 | copper ion transport |
| 5085 | 3.405156691 | down | 0.000065 | 0.000508307 | BI970687 | 670 | 53 | 120 | 44 | 5e−22 | |
| 5086 | 3.402189569 | down | 0.00000117 | 0.0000363 | CD408980 | 524 | 56 | 83 | 67 | 6e−27 | proteolysis and peptidolysis |
| 5087 | 3.402105379 | down | 0.00000035 | 0.0000178 | BI970351 | 780 | 61 | 134 | 45 | 3e−28 | |
| 5088 | 3.401591174 | down | 0.000612251 | 0.002547903 | BG790817 | 479 | 31 | 44 | 70 | 8e−12 | transport |
| 5089 | 3.400317205 | down | 0.0000222 | 0.000241306 | BM094841 | 565 | 95 | 159 | 59 | 3e−50 | regulation of cell cycle |
| 5090 | 3.399708682 | down | 0.00000117 | 0.0000363 | BE821928 | 606 | 80 | 136 | 58 | 5e−44 | electron transport |
| 5091 | 3.399391068 | down | 0.0000011 | 0.0000351 | BQ610503 | 603 | 16 | 45 | 35 | 2.8 | multidrug transport |
| 5092 | 3.397808957 | down | 0.0000157 | 0.000191711 | CD415914 | 589 | 58 | 62 | 93 | 1e−29 | |
| 5093 | 3.39755645 | down | 0.000000532 | 0.0000231 | AW277321 | 603 | 150 | 202 | 74 | 7e−87 | |
| 5094 | 3.397293297 | down | 0.0000122 | 0.000160916 | BM308628 | 606 | 70 | 137 | 51 | 2e−35 | transport |
| 5095 | 3.397216255 | down | 0.0000399 | 0.000361394 | BI784533 | 534 | 52 | 142 | 36 | 9e−10 | regulation of transcription\, DNA- |
| | | | | | | | | | | | dependent |
| 5096 | 3.396981469 | down | 0.0000617 | 0.000489338 | CF807064 | 514 | 59 | 75 | 78 | 6e−30 | carbohydrate metabolism |
| 5097 | 3.394064648 | down | 0.00000487 | 0.0000892 | BU549271 | 626 | 107 | 159 | 67 | 3e−56 | electron transport |
| 5098 | 3.391251397 | down | 0.0000708 | 0.000539677 | BU545074 | 519 | 31 | 58 | 53 | 1e−08 | regulation of transcription\, DNA- |
| | | | | | | | | | | | dependent |
| 5099 | 3.390992916 | down | 0.000000856 | 0.0000307 | BE821217 | 319 | 14 | 20 | 70 | 0.044 | |
| 5100 | 3.386812965 | down | 0.000000363 | 0.0000182 | BG508388 | 483 | 17 | 44 | 38 | 0.020 | metal ion transport |
| 5101 | 3.386717073 | down | 0.00000269 | 0.0000614 | BE583573 | 359 | 10 | 22 | 45 | 0.021 | transport |
| 5102 | 3.385656218 | down | 0.000127354 | 0.000819135 | CD413525 | 664 | 71 | 123 | 57 | 7e−38 | protein folding |
| 5103 | 3.38516935 | down | 0.000000464 | 0.0000214 | AW132856 | 524 | 34 | 68 | 50 | 4e−09 | |
| 5104 | 3.380207508 | down | 0.0000359 | 0.000336872 | CF806536 | 348 | 30 | 49 | 61 | 1e−13 | |
| 5105 | 3.379467827 | down | 0.00000563 | 0.0000976 | AW706013 | 362 | 19 | 33 | 57 | 0.24 | |
| 5106 | 3.378368477 | down | 0.0000156 | 0.00019101 | BE658046 | 692 | 123 | 203 | 60 | 5e−57 | |
| 5107 | 3.377502146 | down | 0.00000364 | 0.0000742 | BI944143 | 472 | 38 | 58 | 65 | 7e−16 | regulation of transcription\, DNA- |
| | | | | | | | | | | | dependent |
| 5108 | 3.376398077 | down | 0.000000382 | 0.0000189 | CD398928 | 524 | 55 | 80 | 68 | 2e−23 | potassium ion transport |
| 5109 | 3.376363823 | down | 0.00000821 | 0.000122383 | BU765881 | 526 | 118 | 174 | 67 | 1e−66 | |
| 5110 | 3.44795301 | down | 0.00000752 | 0.000116249 | BU548684 | 687 | 94 | 136 | 69 | 3e−50 | SferH-2 mRNA for ferritin, iron ion |
| | | | | | | | | | | | transport |
| 5111 | 3.446424207 | down | 0.000373334 | 0.001776643 | AI939229 | 408 | 12 | 12 | 100 | 4.8 | |
| 5112 | 3.446215278 | down | 0.00000583 | 0.0000995 | BI699854 | 420 | 26 | 29 | 89 | 2e−11 | |
| 5113 | 3.445839694 | down | 0.00000392 | 0.0000777 | BE440698 | 427 | 37 | 67 | 55 | 2e−17 | DNA replication |
| 5114 | 3.445692021 | down | 0.000000139 | 0.0000106 | BM528731 | 564 | 130 | 151 | 86 | 2e−74 | |
| 5115 | 3.444139875 | down | 0.00000837 | 0.000124012 | CF808131 | 769 | 142 | 219 | 64 | 6e−81 | electron transport |
| 5116 | 3.442257065 | down | 0.00000288 | 0.000064 | BG510544 | 335 | 12 | 25 | 48 | 0.50 | proteolysis and peptidolysis |
| 5117 | 3.442210974 | down | 0.000374973 | 0.001782411 | AW202151 | 530 | 148 | 181 | 81 | 3e−81 | cellulose biosynthesis |
| 5118 | 3.441407637 | down | 0.0000818 | 0.00059788 | CF807400 | 764 | 72 | 144 | 50 | 4e−25 | gravitropism |
| 5119 | 3.440123514 | down | 0.00000192 | 0.0000497 | AW349281 | 383 | 43 | 46 | 93 | 1e−21 | |
| 5120 | 3.440079081 | down | 0.000000831 | 0.0000302 | AF200824.1 | | | | | | |
| 5121 | 3.635362519 | down | 0.000431136 | 0.001975082 | AB092557.1 | | | | | | cysteine proteinase |
| 5122 | 3.633115341 | down | 0.000000127 | 0.00000996 | BI972263 | 557 | 20 | 38 | 52 | 1e−05 | |
| 5123 | 3.63114143 | down | 0.00000244 | 0.000058 | AW309587 | 663 | 57 | 63 | 90 | 7e−27 | nucleosome assembly |
| 5124 | 3.630116946 | down | 0.00000218 | 0.0000538 | BG237580 | 381 | 17 | 43 | 39 | 0.073 | |
| 5125 | 3.628225959 | down | 0.0000438 | 0.000387644 | CA852328 | 484 | 39 | 77 | 50 | 1e−13 | antibiotic biosynthesis |
| 5126 | 3.627495099 | down | 0.00000628 | 0.000104766 | BG726004 | 517 | 48 | 80 | 60 | 1e−21 | leaf morphogenesis |
| 5127 | 3.625320756 | down | 0.00000154 | 0.0000429 | BG238706 | 430 | 70 | 116 | 60 | 2e−34 | |
| 5128 | 3.623387952 | down | 0.000021 | 0.000231638 | BE021618 | 480 | 129 | 160 | 80 | 1e−72 | adaxial/abaxial pattern formation |
| 5129 | 3.621240857 | down | 0.0000339 | 0.00032462 | AW119750 | 346 | 14 | 18 | 77 | 0.076 | protein amino acid phosphorylation |
| 5130 | 3.62096451 | down | 0.000000989 | 0.0000335 | CD412522 | 639 | 52 | 79 | 65 | 2e−29 | |
| 5131 | 3.619751142 | down | 0.000599603 | 0.002509287 | BQ629904 | 616 | 62 | 145 | 42 | 7e−31 | |
| 5132 | 3.619465088 | down | 0.00000219 | 0.000054 | CD487575 | 647 | 82 | 82 | 100 | 1e−40 | nucleosome assembly |
| 5133 | 3.619321873 | down | 0.00000145 | 0.0000412 | AI736292 | 233 | 9 | 15 | 60 | 5.4 | flagellum biogenesis |
| 5134 | 3.618088347 | down | 0.000000436 | 0.0000205 | BI971547 | 520 | 20 | 70 | 28 | 1.2 | electron transport |
| 5135 | 3.617401498 | down | 0.00000324 | 0.0000689 | BU546154 | 583 | 93 | 176 | 52 | 4e−53 | metabolism |
| 5136 | 3.615413408 | down | 0.00000393 | 0.000078 | CD395653 | 630 | 45 | 141 | 31 | 1e−08 | |
| 5137 | 3.612458651 | down | 0.0000681 | 0.000524974 | CF806954 | 364 | 34 | 38 | 89 | 3e−15 | regulation of translation |
| 5138 | 3.610622061 | down | 0.00000553 | 0.0000966 | BU548251 | 585 | 36 | 84 | 42 | 1e−15 | regulation of transcription\, DNA- |
| | | | | | | | | | | | dependent |
| 5139 | 3.60996409 | down | 0.00000767 | 0.000117626 | AI442632 | 325 | 42 | 52 | 80 | 7e−19 | |
| 5140 | 3.609183405 | down | 0.000000268 | 0.000015 | BQ079922 | 555 | 29 | 56 | 51 | 3e−14 | regulation of transcription\, DNA- |
| | | | | | | | | | | | dependent |
| 5141 | 3.60847004 | down | 0.00000314 | 0.0000678 | BE659531 | 676 | 45 | 87 | 51 | 5e−18 | protein amino acid phosphorylation |
| 5142 | 3.607959086 | down | 0.00000326 | 0.0000692 | CA785949 | 606 | 78 | 201 | 38 | 2e−37 | |
| 5143 | 3.606328307 | down | 0.0000745 | 0.000559008 | BG157768 | 656 | 170 | 218 | 77 | 8e−95 | |
| 5144 | 3.605909807 | down | 0.0000115 | 0.000153998 | BI972063 | 558 | 16 | 44 | 36 | 9.2 | |
| 5145 | 3.603652542 | down | 0.000228932 | 0.00124419 | CF807400 | 764 | 72 | 144 | 50 | 4e−25 | gravitropism |
| 5146 | 3.603064533 | down | 0.00000413 | 0.0000804 | AW101266 | 425 | 67 | 137 | 48 | 8e−25 | |
| 5147 | 3.60298532 | down | 0.0000228 | 0.000246214 | BG508133 | 414 | 23 | 70 | 32 | 9e−05 | proteolysis and peptidolysis |
| 5148 | 3.602051318 | down | 0.00000439 | 0.0000836 | BG046595 | 519 | 104 | 176 | 59 | 5e−55 | electron transport |
| 5149 | 3.598542451 | down | 0.0000193 | 0.000218938 | BE023656 | 496 | 62 | 140 | 44 | 1e−32 | proteolysis and peptidolysis |
| 5150 | 3.59816925 | down | 0.00000425 | 0.000082 | CF806108 | 618 | 101 | 163 | 61 | 8e−46 | |
| 5151 | 3.597797577 | down | 8.63E−08 | 0.00000795 | BM522624 | 378 | 12 | 32 | 37 | 0.46 | |
| 5152 | 3.596519675 | down | 0.000109308 | 0.000732109 | BG652533 | 355 | 18 | 47 | 38 | 0.39 | transport |
| 5153 | 3.594583411 | down | 0.00000286 | 0.0000637 | BI471633 | 504 | 94 | 117 | 80 | 1e−49 | |
| 5154 | 3.591149366 | down | 0.00000462 | 0.000086 | BG157412 | 474 | 82 | 122 | 67 | 4e−50 | |
| 5155 | 3.590383197 | down | 0.00000751 | 0.000116189 | CD399193 | 372 | 17 | 30 | 56 | 8e−06 | |
| 5156 | 3.589903749 | down | 0.00000369 | 0.0000751 | BI970240 | 772 | 124 | 200 | 62 | 1e−67 | transport |
| 5157 | 3.586480422 | down | 0.00000234 | 0.0000564 | AW310916 | 535 | 55 | 95 | 57 | 1e−29 | basic amino acid transport |
| 5158 | 3.585844127 | down | 0.000000543 | 0.0000235 | BI972302 | | | | | | |
| 5159 | 3.584784796 | down | 0.00000195 | 0.0000501 | BF070874 | 462 | 18 | 39 | 46 | 0.011 | |
| 5160 | 3.584381252 | down | 0.0000198 | 0.000222658 | AW307023 | 360 | 12 | 20 | 60 | 4.4 | |
| 5161 | 3.58422524 | down | 0.00000324 | 0.0000689 | BI970282 | 649 | 58 | 107 | 54 | 9e−27 | |
| 5162 | 3.583321512 | down | 0.000000112 | 0.0000092 | AW201116 | 365 | 54 | 122 | 44 | 8e−24 | |
| 5163 | 3.582705168 | down | 0.00000217 | 0.0000536 | BQ627543 | 397 | 18 | 58 | 31 | 0.10 | beta-tubulin, defense response |
| 5164 | 3.581365903 | down | 0.0000657 | 0.000511713 | CD401169 | 618 | 8 | 20 | 40 | 5.0 | mRNA splicing |
| 5165 | 3.581106018 | down | 0.0000101 | 0.000141584 | AI988142 | 647 | 51 | 112 | 45 | 8e−25 | protein amino acid phosphorylation |
| 5166 | 3.580659528 | down | 0.00000124 | 0.0000373 | BU544647 | 576 | 57 | 120 | 47 | 2e−24 | protein amino acid phosphorylation |
| 5167 | 3.579929715 | down | 0.0000578 | 0.000468361 | AW596159 | 392 | 38 | 58 | 65 | 1e−17 | |
| 5168 | 3.579033563 | down | 0.00000159 | 0.0000439 | CD406455 | 567 | 77 | 109 | 70 | 2e−44 | |
| 5169 | 3.578337963 | down | 0.000000682 | 0.000027 | CD392967 | 651 | 78 | 174 | 44 | 1e−37 | |
| 5170 | 3.575924844 | down | 0.000130118 | 0.000831223 | CD397826 | 378 | 18 | 35 | 51 | 0.005 | regulation of transcription |
| 5171 | 3.575730885 | down | 0.0000385 | 0.000352804 | AW101688 | 416 | 38 | 54 | 70 | 1e−16 | carbohydrate metabolism |
| 5172 | 3.574520782 | down | 0.00000399 | 0.0000785 | AW349905 | 713 | 68 | 113 | 60 | 1e−31 | cell wall modification (sensu |
| | | | | | | | | | | | Magnoliophyta) |
| 5173 | 3.573391336 | down | 0.000000187 | 0.0000124 | BU763206 | 590 | 100 | 181 | 55 | 6e−56 | |
| 5174 | 3.572530461 | down | 0.000000728 | 0.0000281 | BE329505 | 412 | 45 | 54 | 83 | 5e−24 | |
| 5175 | 3.571657488 | down | 0.001219596 | 0.004200129 | BI701509 | 541 | 39 | 66 | 59 | 3e−14 | |
| 5176 | 3.570285396 | down | 0.00000315 | 0.0000678 | BU082396 | 478 | 99 | 159 | 62 | 2e−51 | |
| 5177 | 3.56953948 | down | 0.000319714 | 0.001589024 | BU549984 | 647 | 92 | 124 | 74 | 2e−52 | electron transport |
| 5178 | 3.568856781 | down | 0.000153815 | 0.000933008 | BF595347 | | | | | | |
| 5179 | 3.568716255 | down | 0.0000097 | 0.000137393 | BI967570 | 513 | 14 | 40 | 35 | 1.2 | |
| 5180 | 3.568374562 | down | 0.000000236 | 0.0000142 | BU926323 | 634 | 127 | 169 | 75 | 2e−71 | carbohydrate metabolism |
| 5181 | 3.5681995 | down | 0.0001562 | 0.000944277 | BE210622 | 574 | 121 | 192 | 63 | 1e−65 | proteolysis and peptidolysis |
| 5182 | 3.566814479 | down | 0.0000362 | 0.000338753 | BE473596 | 577 | 80 | 105 | 76 | 1e−41 | histidine biosynthesis |
| 5183 | 3.566775667 | down | 0.0000256 | 0.000266873 | CA819683 | | | | | | |
| 5184 | 3.566601231 | down | 0.00000246 | 0.0000583 | BM309584 | 602 | 99 | 162 | 61 | 1e−53 | threonine catabolism |
| 5185 | 3.563651677 | down | 0.000000221 | 0.0000137 | BE802754 | 466 | 27 | 64 | 42 | 2e−07 | |
| 5186 | 3.557602462 | down | 0.000124454 | 0.00080669 | BI787925 | 570 | 111 | 188 | 59 | 6e−60 | |
| 5187 | 3.557525028 | down | 0.0000122 | 0.000161084 | AW459996 | 441 | 21 | 72 | 29 | 1.2 | |
| 5188 | 3.556359684 | down | 0.0000044 | 0.0000838 | CD394315 | 534 | 21 | 69 | 30 | 5.0 | |
| 5189 | 3.556102503 | down | 0.00000146 | 0.0000413 | BE473384 | 675 | 124 | 206 | 60 | 1e−65 | |
| 5190 | 3.555015466 | down | 0.00000181 | 0.0000478 | BE611807 | 288 | 58 | 90 | 64 | 6e−33 | |
| 5191 | 3.554987426 | down | 0.00000212 | 0.000053 | BQ611733 | 426 | 42 | 106 | 39 | 5e−17 | protein amino acid phosphorylation |
| 5192 | 3.554696053 | down | 0.000533349 | 0.002308029 | D86351.1 | | | | | | CYP93A2 mRNA for cytochrome P-450 |
| 5193 | 3.554299196 | down | 0.000000352 | 0.0000179 | BU546934 | 645 | 70 | 107 | 65 | 7e−35 | cell wall catabolism |
| 5194 | 3.552444546 | down | 0.0000496 | 0.000420793 | CD408242 | 634 | 102 | 124 | 82 | 2e−56 | metal ion transport |
| 5195 | 3.551398814 | down | 0.0000115 | 0.000154012 | BG725901 | 251 | 67 | 83 | 80 | 9e−38 | |
| 5196 | 3.55135089 | down | 0.000035 | 0.000331437 | BI892886 | 359 | 10 | 13 | 76 | 1.5 | |
| 5197 | 3.550734692 | down | 0.000126929 | 0.000817791 | BE819998 | 827 | 72 | 160 | 45 | 1e−24 | |
| 5198 | 3.548135308 | down | 0.0000434 | 0.000384728 | CF806507 | 617 | 144 | 204 | 70 | 1e−81 | cell elongation |
| 5199 | 3.546867138 | down | 0.00000228 | 0.0000554 | BG651852 | 385 | 76 | 122 | 62 | 3e−42 | |
| 5200 | 3.546641993 | down | 0.00000274 | 0.000062 | BI317274 | 508 | 107 | 170 | 62 | 1e−58 | proline dehydrogenase, glutamate |
| | | | | | | | | | | | biosynthesis |
| 5201 | 3.546617393 | down | 0.0000389 | 0.000354871 | AB086391.1 | | | | | | simillar to Arabidopsis thaliana putative |
| | | | | | | | | | | | disease resistance protein |
| 5202 | 3.545774562 | down | 0.000179695 | 0.001041454 | BU548312 | 559 | 41 | 41 | 100 | 4e−17 | L-serine biosynthesis |
| 5203 | 3.545356763 | down | 0.0000178 | 0.000207804 | BM526911 | 432 | 49 | 68 | 72 | 2e−24 | response to pest/pathogen/parasite |
| 5204 | 3.544648698 | down | 0.001506935 | 0.004888053 | CF808286 | | | | | | |
| 5205 | 3.543776504 | down | 0.001458583 | 0.004772969 | BQ081227 | 338 | 17 | 42 | 40 | 0.29 | |
| 5206 | 3.543657549 | down | 0.000000777 | 0.0000291 | CD411972 | 690 | 77 | 119 | 64 | 7e−41 | |
| 5207 | 3.542022324 | down | 0.000000241 | 0.0000143 | AW348801 | 565 | 38 | 66 | 57 | 9e−14 | cytokinesis by cell plate formation |
| 5208 | 3.540141587 | down | 0.000000492 | 0.000022 | AW349390 | 705 | 82 | 163 | 50 | 4e−42 | protein amino acid phosphorylation |
| 5209 | 3.539394859 | down | 0.00000445 | 0.0000844 | BI316936 | | | | | | |
| 5210 | 3.539216759 | down | 0.0000192 | 0.00021874 | BI970193 | | | | | | |
| 5211 | 3.539122571 | down | 0.0000443 | 0.000389852 | AW759674 | 583 | 73 | 120 | 60 | 7e−38 | |
| 5212 | 3.538824081 | down | 0.000000774 | 0.0000291 | BG046811 | 654 | 53 | 125 | 42 | 4e−22 | |
| 5213 | 3.53672399 | down | 0.000000728 | 0.0000281 | BI471633 | 504 | 94 | 117 | 80 | 1e−49 | |
| 5214 | 3.536402299 | down | 0.00000116 | 0.0000363 | CD392813 | 611 | 62 | 141 | 43 | 5e−24 | |
| 5215 | 3.535163655 | down | 0.0000254 | 0.000265783 | BG651854 | 494 | 40 | 75 | 53 | 1e−14 | G beta-like protein |
| 5216 | 3.533812804 | down | 0.000054 | 0.000446559 | BU083321 | 557 | 23 | 49 | 46 | 7e−08 | |
| 5217 | 3.532060987 | down | 0.000000215 | 0.0000136 | BI969186 | 773 | 94 | 194 | 48 | 2e−45 | |
| 5218 | 3.531430776 | down | 0.0000494 | 0.0004195 | AW310270 | 545 | 44 | 98 | 44 | 3e−22 | |
| 5219 | 3.53053173 | down | 0.001148457 | 0.004020294 | CD396375 | 694 | 74 | 164 | 45 | 4e−36 | protein folding |
| 5220 | 3.530525666 | down | 0.0000418 | 0.000374525 | BE823592 | 644 | 22 | 54 | 40 | 2e−06 | |
| 5221 | 3.530401227 | down | 0.000084 | 0.00060854 | BM143981 | 537 | 49 | 58 | 84 | 1e−23 | regulation of transcription |
| 5222 | 3.528999123 | down | 0.000000256 | 0.0000148 | BE821073 | 573 | 71 | 157 | 45 | 1e−24 | regulation of transcription |
| 5223 | 3.528020838 | down | 0.0000162 | 0.00019591 | AY113187.2 | | | | | | diphenol oxidase laccase |
| 5224 | 3.520873145 | down | 0.000000359 | 0.0000181 | BI424960 | 442 | 18 | 44 | 40 | 0.40 | proteolysis and peptidolysis |
| 5225 | 3.520583348 | down | 0.000000177 | 0.0000119 | CF809254 | 257 | 10 | 11 | 90 | 0.85 | |
| 5226 | 3.519654157 | down | 0.00000804 | 0.00012101 | BQ627853 | 429 | 61 | 102 | 59 | 6e−34 | |
| 5227 | 3.519251935 | down | 0.00000252 | 0.0000592 | CF808853 | 572 | 52 | 136 | 38 | 9e−19 | regulation of transcription\, DNA- |
| | | | | | | | | | | | dependent |
| 5228 | 3.519036076 | down | 0.000178138 | 0.001037068 | BE807239 | 346 | 11 | 34 | 32 | 2.5 | response to oxidative stress |
| 5229 | 3.518359877 | down | 0.00000279 | 0.0000628 | BG652383 | 524 | 28 | 39 | 71 | 7e−13 | |
| 5230 | 3.517309948 | down | 0.0000712 | 0.00054144 | BE584385 | 678 | 61 | 201 | 30 | 1e−18 | |
| 5231 | 3.513961899 | down | 0.0000259 | 0.000269286 | BF324343 | 365 | 14 | 35 | 40 | 1.2 | ubiquitin-dependent protein catabolism |
| 5232 | 3.513010687 | down | 0.000000223 | 0.0000138 | CA820109 | 403 | 23 | 25 | 92 | 1e−08 | cell-cell signaling |
| 5233 | 3.512110975 | down | 0.000000588 | 0.0000245 | BM188774 | 502 | 19 | 27 | 70 | 1e−06 | |
| 5234 | 3.511533719 | down | 0.000000236 | 0.0000141 | AW570625 | 352 | 82 | 117 | 70 | 7e−40 | |
| 5235 | 3.506528633 | down | 0.000134389 | 0.000849706 | CA784390 | 734 | 111 | 225 | 49 | 6e−54 | |
| 5236 | 3.506360674 | down | 0.000228356 | 0.001241957 | CF806310 | 505 | 103 | 167 | 61 | 2e−56 | proteolysis and peptidolysis |
| 5237 | 3.505204373 | down | 0.00000264 | 0.0000608 | BI970635 | 791 | 110 | 130 | 84 | 3e−64 | electron transport |
| 5238 | 3.502551677 | down | 0.000000974 | 0.0000331 | BE657833 | | | | | | |
| 5239 | 3.501862831 | down | 0.00000788 | 0.000119427 | BE475461 | 382 | 10 | 23 | 43 | 8.9 | |
| 5240 | 3.501723773 | down | 0.000000606 | 0.0000249 | CF808300 | 500 | 139 | 166 | 83 | 9e−81 | electron transport |
| 5241 | 3.497211342 | down | 0.000687229 | 0.002767307 | CD409280 | 654 | 69 | 100 | 69 | 1e−40 | |
| 5242 | 3.493906797 | down | 0.0000039 | 0.0000775 | CD396246 | 731 | 72 | 146 | 49 | 4e−36 | metabolism |
| 5243 | 3.493209008 | down | 0.000000996 | 0.0000336 | BM307190 | 279 | 13 | 44 | 29 | 1.9 | |
| 5244 | 3.492529604 | down | 0.0000179 | 0.00020806 | CF808052 | 560 | 117 | 150 | 78 | 3e−61 | protein biosynthesis |
| 5245 | 3.49061611 | down | 0.00000904 | 0.000130701 | BU548038 | 639 | 91 | 137 | 66 | 6e−50 | metabolism |
| 5246 | 3.486550766 | down | 0.00000572 | 0.0000985 | BU550899 | 735 | 154 | 204 | 75 | 2e−88 | glycerol metabolism |
| 5247 | 3.485987582 | down | 0.00000031 | 0.0000167 | BM307957 | 374 | 9 | 24 | 37 | 4.9 | electron transport |
| 5248 | 3.485978549 | down | 0.000021 | 0.000231593 | CF806853 | 150 | 11 | 25 | 44 | 3.2 | |
| 5249 | 3.48567324 | down | 0.000000872 | 0.000031 | CA820135 | 431 | 24 | 36 | 66 | 8e−07 | |
| 5250 | 3.483707509 | down | 0.00000498 | 0.0000907 | BG789974 | 527 | 81 | 102 | 79 | 6e−49 | |
| 5251 | 3.482813655 | down | 0.00000114 | 0.000036 | BI317149 | 401 | 58 | 121 | 47 | 2e−27 | threonine catabolism |
| 5252 | 3.482648714 | down | 0.00000148 | 0.0000417 | AF039027.1 | | | | | | cationic peroxidase 2 |
| 5253 | 3.481426335 | down | 0.000000677 | 0.0000269 | BU765157 | 488 | 20 | 56 | 35 | 0.29 | |
| 5254 | 3.437160008 | down | 0.000035 | 0.000331784 | CA800565 | | | | | | |
| 5255 | 3.436833377 | down | 0.00000374 | 0.0000754 | AW568133 | 412 | 29 | 30 | 96 | 9e−13 | |
| 5256 | 3.435570637 | down | 0.0000115 | 0.000154229 | BQ134043 | 404 | 122 | 134 | 91 | 8e−74 | |
| 5257 | 3.432208088 | down | 0.00000937 | 0.000133599 | BU549658 | 709 | 77 | 109 | 70 | 9e−41 | protein catabolism |
| 5258 | 3.431387444 | down | 0.0000166 | 0.000198349 | BQ454195 | 450 | 40 | 71 | 56 | 9e−12 | regulation of transcription\, DNA- |
| | | | | | | | | | | | dependent |
| 5259 | 3.429404716 | down | 0.0000025 | 0.000059 | BM568170 | 407 | 22 | 34 | 64 | 1e−08 | |
| 5260 | 3.428749602 | down | 0.00000507 | 0.0000918 | BI785026 | 522 | 62 | 133 | 46 | 3e−24 | |
| 5261 | 3.423258552 | down | 0.0000171 | 0.000202434 | AI443304 | 388 | 26 | 30 | 86 | 4e−09 | metabolism |
| 5262 | 3.423142513 | down | 0.0000335 | 0.000321984 | CA783454 | 454 | 14 | 51 | 27 | 2.7 | transport |
| 5263 | 3.421753062 | down | 0.000660951 | 0.002694086 | BE823048 | 609 | 64 | 94 | 68 | 1e−32 | regulation of transcription\, DNA- |
| | | | | | | | | | | | dependent |
| 5264 | 3.418670288 | down | 0.00000265 | 0.0000609 | CD417025 | 356 | 41 | 55 | 74 | 1e−20 | |
| 5265 | 3.418437926 | down | 0.00000185 | 0.0000483 | CD395009 | 482 | 24 | 65 | 36 | 0.17 | |
| 5266 | 3.417821395 | down | 0.00000134 | 0.0000393 | BI968693 | 711 | 116 | 135 | 85 | 2e−63 | calmodulin |
| 5267 | 3.41644024 | down | 0.000000446 | 0.0000208 | AI431186 | 446 | 16 | 34 | 47 | 2e−04 | |
| 5268 | 3.415609615 | down | 0.000260718 | 0.00136907 | BM140147 | 192 | 12 | 27 | 44 | 7.1 | |
| 5269 | 3.415422515 | down | 0.0000182 | 0.000210043 | BE823282 | 622 | 92 | 138 | 66 | 3e−47 | dicarboxylic acid transport |
| 5270 | 3.415282842 | down | 0.000000477 | 0.0000217 | BM523097 | 324 | 64 | 83 | 77 | 1e−29 | |
| 5271 | 3.413607407 | down | 0.0000112 | 0.000151499 | AW706106 | 474 | 13 | 14 | 92 | 0.019 | cation transport |
| 5272 | 3.376282929 | down | 0.0000308 | 0.000303081 | CK606089 | 538 | 69 | 139 | 49 | 1e−32 | |
| 5273 | 3.375607176 | down | 0.000000464 | 0.0000214 | BG881453 | 417 | 42 | 60 | 70 | 2e−19 | protein amino acid phosphorylation |
| 5274 | 3.375132543 | down | 0.0000288 | 0.000289322 | BQ094516 | 535 | 141 | 178 | 79 | 7e−82 | cell wall modification (sensu |
| | | | | | | | | | | | Magnoliophyta) |
| 5275 | 3.374906643 | down | 0.00000431 | 0.0000827 | AW780837 | 436 | 96 | 111 | 86 | 2e−51 | protein amino acid phosphorylation |
| 5276 | 3.371798652 | down | 0.00000903 | 0.000130508 | BU549198 | 701 | 102 | 172 | 59 | 1e−55 | electron transport |
| 5277 | 3.36936027 | down | 0.00000129 | 0.0000382 | AW350600 | 727 | 67 | 166 | 40 | 1e−28 | protein amino acid phosphorylation |
| 5278 | 3.36683205 | down | 0.00000126 | 0.0000378 | BE805868 | 550 | 103 | 154 | 66 | 2e−50 | |
| 5279 | 3.365228826 | down | 0.000150926 | 0.000920685 | AW755809 | 252 | 33 | 77 | 42 | 8e−15 | protein amino acid phosphorylation |
| 5280 | 3.363617006 | down | 0.00000241 | 0.0000576 | BG046128 | 368 | 16 | 44 | 36 | 2.8 | |
| 5281 | 3.362564175 | down | 0.0000142 | 0.000178829 | CA853360 | 645 | 145 | 197 | 73 | 6e−81 | proteolysis and peptidolysis |
| 5282 | 3.362330313 | down | 0.00000212 | 0.000053 | AW706795 | 551 | 68 | 99 | 68 | 7e−32 | |
| 5283 | 3.360037708 | down | 0.0000371 | 0.000344013 | BQ627483 | 519 | 77 | 120 | 64 | 1e−40 | carbohydrate metabolism |
| 5284 | 3.358938575 | down | 0.00000136 | 0.0000397 | CD407697 | 589 | 87 | 89 | 97 | 2e−43 | nucleosome assembly |
| 5285 | 3.357288406 | down | 0.00000932 | 0.000133084 | BF424943 | 662 | 95 | 164 | 57 | 2e−43 | |
| 5286 | 3.356963285 | down | 0.00000496 | 0.0000905 | BG047180 | 469 | 54 | 74 | 72 | 6e−27 | |
| 5287 | 3.356739455 | down | 0.000000781 | 0.0000291 | BU763753 | 449 | 19 | 53 | 35 | 0.14 | transport |
| 5288 | 3.356237586 | down | 0.001253834 | 0.0042847 | AW397248 | 454 | 62 | 68 | 91 | 1e−28 | regulation of meristem organization |
| 5289 | 3.355930874 | down | 0.00000161 | 0.0000441 | BM527540 | 427 | 29 | 41 | 70 | 9e−13 | |
| 5290 | 3.353415514 | down | 0.0000303 | 0.000300323 | CD408242 | 634 | 102 | 124 | 82 | 2e−56 | metal ion transport |
| 5291 | 3.352930299 | down | 0.000000832 | 0.0000302 | BE209646 | 278 | 7 | 20 | 35 | 7.1 | |
| 5292 | 3.352041787 | down | 0.0000233 | 0.000249858 | CD406598 | 429 | 52 | 91 | 57 | 3e−23 | |
| 5293 | 3.351994416 | down | 0.0000512 | 0.000429832 | CD393774 | 542 | 30 | 106 | 28 | 0.015 | |
| 5294 | 3.351920419 | down | 0.0000294 | 0.000293498 | BF424989 | 396 | 42 | 75 | 56 | 2e−15 | regulation of transcription |
| 5295 | 3.351734761 | down | 0.000355525 | 0.001714012 | CF806688 | 716 | 185 | 217 | 85 | e−109 | |
| 5296 | 3.351257259 | down | 0.00000931 | 0.000132901 | BG362785 | 523 | 58 | 120 | 48 | 6e−22 | |
| 5297 | 3.348203949 | down | 0.00000311 | 0.0000674 | BQ610093 | 430 | 122 | 143 | 85 | 7e−69 | regulation of cell cycle |
| 5298 | 3.34540829 | down | 0.0000824 | 0.00060077 | CF808910 | 537 | 136 | 181 | 75 | 3e−81 | galactose metabolism |
| 5299 | 3.343853589 | down | 0.0000169 | 0.000200669 | BF009966 | 440 | 45 | 104 | 43 | 1e−15 | defense response |
| 5300 | 3.34300045 | down | 0.0000204 | 0.000227142 | BE020773 | 423 | 80 | 141 | 56 | 3e−37 | N-terminal protein myristoylation |
| 5301 | 3.342612104 | down | 0.000000779 | 0.0000291 | BQ612677 | 262 | 10 | 21 | 47 | 7.2 | |
| 5302 | 3.33975628 | down | 0.0000103 | 0.000142582 | CD413054 | 428 | 22 | 43 | 51 | 4e−09 | |
| 5303 | 3.33971669 | down | 0.000000185 | 0.0000123 | BE658303 | 782 | 99 | 131 | 75 | 2e−55 | |
| 5304 | 3.339460389 | down | 0.0000104 | 0.000143934 | BE609705 | 576 | 29 | 92 | 31 | 3e−04 | |
| 5305 | 3.339182041 | down | 0.000000254 | 0.0000148 | BE023327 | 416 | 54 | 88 | 61 | 2e−25 | peptidoglycan metabolism |
| 5306 | 3.339100209 | down | 0.000399297 | 0.00186504 | AW278477 | | | | | | |
| 5307 | 3.339058381 | down | 0.000000765 | 0.0000289 | BI497597 | 533 | 69 | 98 | 70 | 5e−40 | |
| 5308 | 3.338134161 | down | 0.00000701 | 0.000111286 | CA936776 | 420 | 16 | 41 | 39 | 0.46 | |
| 5309 | 3.33614517 | down | 0.00000128 | 0.0000382 | BG362734 | 563 | 53 | 129 | 41 | 1e−21 | |
| 5310 | 3.335886293 | down | 0.00000483 | 0.0000887 | BQ298754 | 273 | 14 | 34 | 41 | 0.49 | regulation of transcription\, DNA- |
| | | | | | | | | | | | dependent |
| 5311 | 3.334928896 | down | 0.000000975 | 0.0000331 | BE822507 | 776 | 58 | 103 | 56 | 3e−21 | |
| 5312 | 3.334811034 | down | 0.0000182 | 0.000210491 | BU544114 | 597 | 60 | 100 | 60 | 7e−30 | |
| 5313 | 3.333193887 | down | 0.0000109 | 0.000149019 | BE657554 | 731 | 68 | 96 | 70 | 9e−34 | amino acid transport |
| 5314 | 3.332743662 | down | 0.0000327 | 0.000316007 | BE440266 | 425 | 31 | 43 | 72 | 2e−12 | |
| 5315 | 3.331037554 | down | 0.00000227 | 0.0000554 | BM107812 | 598 | 85 | 108 | 78 | 2e−46 | transport |
| 5316 | 3.330958648 | down | 0.000285812 | 0.001465449 | CD390874 | 572 | 20 | 38 | 52 | 0.016 | |
| 5317 | 3.33059017 | down | 0.00000123 | 0.0000372 | BI969361 | 798 | 120 | 139 | 86 | 2e−66 | |
| 5318 | 3.328592987 | down | 0.0000074 | 0.0001153 | BE022059 | 411 | 67 | 137 | 48 | 6e−30 | |
| 5319 | 3.327188175 | down | 0.000000132 | 0.0000102 | BQ741052 | 569 | 54 | 146 | 36 | 7e−16 | metabolism |
| 5320 | 3.325091551 | down | 0.00000771 | 0.000117799 | BQ610148 | 553 | 100 | 185 | 54 | 5e−54 | |
| 5321 | 3.323725837 | down | 0.00000577 | 0.0000989 | BU546284 | 586 | 15 | 56 | 26 | 7.8 | transport |
| 5322 | 3.323135587 | down | 0.0000337 | 0.000323272 | BQ079740 | 407 | 8 | 26 | 30 | 8.2 | |
| 5323 | 3.323020888 | down | 0.000000314 | 0.0000167 | AW102323 | 749 | 31 | 55 | 56 | 2e−12 | |
| 5324 | 3.322862461 | down | 0.000000449 | 0.000021 | CF807592 | 568 | 145 | 163 | 88 | 1e−77 | |
| 5325 | 3.321557288 | down | 0.00000356 | 0.0000731 | CA819674 | 398 | 16 | 19 | 84 | 2e−05 | response to oxidative stress |
| 5326 | 3.320319294 | down | 0.00000215 | 0.0000534 | BM527366 | 519 | 41 | 62 | 66 | 9e−23 | G-protein coupled receptor protein |
| | | | | | | | | | | | signaling pathway |
| 5327 | 3.319297445 | down | 0.00000843 | 0.000124507 | CF808053 | 538 | 72 | 103 | 69 | 4e−39 | |
| 5328 | 3.317660249 | down | 0.0000053 | 0.0000942 | BI971068 | 796 | 54 | 118 | 45 | 1e−19 | |
| 5329 | 3.315357878 | down | 0.000000116 | 0.00000943 | BG362816 | 538 | 96 | 180 | 53 | 4e−54 | carbohydrate metabolism |
| 5330 | 3.314160891 | down | 0.000121559 | 0.00079243 | CA782625 | 760 | 86 | 116 | 74 | 7e−48 | protein amino acid dephosphorylation |
| 5331 | 3.314139962 | down | 0.00000322 | 0.0000687 | AW458428 | 366 | 15 | 40 | 37 | 1.2 | cell death |
| 5332 | 3.314104685 | down | 0.000000441 | 0.0000207 | BM567833 | 430 | 41 | 54 | 75 | 1e−17 | electron transport |
| 5333 | 3.314073444 | down | 0.00000129 | 0.0000382 | BE210212 | 500 | 103 | 130 | 79 | 2e−57 | |
| 5334 | 3.313518511 | down | 0.00000673 | 0.000108581 | BI784541 | 552 | 24 | 47 | 51 | 7e−05 | |
| 5335 | 3.312390831 | down | 0.000000173 | 0.0000118 | CF808517 | 557 | 102 | 160 | 63 | 1e−57 | |
| 5336 | 3.311170574 | down | 0.000000395 | 0.0000192 | BU550052 | 703 | 32 | 64 | 50 | 1e−12 | |
| 5337 | 3.310681971 | down | 0.0000236 | 0.000252459 | BE475515 | 563 | 134 | 187 | 71 | 4e−86 | |
| 5338 | 3.310157967 | down | 0.0000622 | 0.000492795 | BM522902 | 433 | 21 | 83 | 25 | 3.2 | |
| 5339 | 3.306078569 | down | 0.000152211 | 0.000926041 | CF807766 | 696 | 50 | 220 | 22 | 3e−06 | |
| 5340 | 3.305889083 | down | 0.00000071 | 0.0000276 | AW734257 | 356 | 16 | 16 | 100 | 0.009 | cell differentiation |
| 5341 | 3.304475848 | down | 0.0000229 | 0.000246966 | AW733336 | 578 | 97 | 192 | 50 | 5e−47 | electron transport |
| 5342 | 3.299069636 | down | 0.00000115 | 0.0000361 | AW471809 | 463 | 30 | 56 | 53 | 2e−07 | |
| 5343 | 3.299064511 | down | 0.00000055 | 0.0000236 | AW309198 | 434 | 94 | 144 | 65 | 4e−47 | biological_process unknown |
| 5344 | 3.299000978 | down | 0.0000113 | 0.000152786 | BE805396 | 572 | 110 | 195 | 56 | 5e−50 | regulation of transcription\, DNA- |
| | | | | | | | | | | | dependent |
| 5345 | 3.297734402 | down | 0.000034 | 0.000325184 | BI316016 | 216 | 17 | 43 | 39 | 1.1 | |
| 5346 | 3.297673384 | down | 0.00000494 | 0.0000903 | BE210339 | 516 | 31 | 60 | 51 | 2e−10 | protein biosynthesis |
| 5347 | 3.295870655 | down | 0.000000266 | 0.0000149 | AW350019 | 795 | 122 | 231 | 52 | 5e−62 | |
| 5348 | 3.294384474 | down | 0.00000633 | 0.000105028 | BI699438 | 490 | 27 | 89 | 30 | 0.026 | two-component signal transduction |
| | | | | | | | | | | | system (phosphorelay) |
| 5349 | 3.29248306 | down | 0.000015 | 0.000185595 | BG789513 | 417 | 34 | 37 | 91 | 1e−12 | |
| 5350 | 3.291989118 | down | 0.0000477 | 0.000410104 | BM187682 | 570 | 136 | 195 | 69 | 2e−73 | protein amino acid phosphorylation |
| 5351 | 3.291771353 | down | 0.000021 | 0.000231685 | BM524404 | 547 | 146 | 163 | 89 | 2e−82 | regulation of transcription\, DNA- |
| | | | | | | | | | | | dependent |
| 5352 | 3.291385672 | down | 0.0000108 | 0.000147365 | BE658252 | 713 | 39 | 49 | 79 | 2e−20 | |
| 5353 | 3.289259547 | down | 0.000139882 | 0.00087325 | BI969411 | 770 | 98 | 152 | 64 | 1e−59 | |
| 5354 | 3.286728483 | down | 0.00000116 | 0.0000363 | BG238637 | 590 | 23 | 36 | 63 | 1e−08 | anion transport |
| 5355 | 3.286400443 | down | 0.0000177 | 0.000206947 | BI315572 | 494 | 103 | 165 | 62 | 7e−56 | |
| 5356 | 3.28472556 | down | 0.000262691 | 0.00137653 | AW348555 | 695 | 97 | 151 | 64 | 2e−51 | protein amino acid phosphorylation |
| 5357 | 3.284543036 | down | 0.0000508 | 0.000427573 | CF805832 | 365 | 16 | 21 | 76 | 0.006 | |
| 5358 | 3.28288618 | down | 0.00000653 | 0.000106503 | BM143204 | 464 | 109 | 188 | 57 | 3e−51 | potassium ion transport |
| 5359 | 3.282736333 | down | 0.000000622 | 0.0000253 | AW781747 | 559 | 103 | 156 | 66 | 4e−57 | |
| 5360 | 3.28133079 | down | 0.000000571 | 0.0000242 | BF070459 | 615 | 133 | 193 | 68 | 2e−72 | cell differentiation |
| 5361 | 3.201355452 | down | 0.00000393 | 0.000078 | BM178585 | 539 | 88 | 104 | 84 | 9e−53 | |
| 5362 | 3.201298596 | down | 0.0000066 | 0.000107228 | BG237912 | 325 | 14 | 17 | 82 | 0.020 | carbohydrate metabolism |
| 5363 | 3.200343327 | down | 0.000000401 | 0.0000194 | AW310476 | 645 | 13 | 38 | 34 | 0.82 | |
| 5364 | 3.199784804 | down | 0.001059598 | 0.00380085 | BE657601 | 761 | 101 | 191 | 52 | 2e−52 | |
| 5365 | 3.198448592 | down | 0.0000298 | 0.000295988 | CA784263 | 762 | 194 | 250 | 77 | e−108 | |
| 5366 | 3.197523005 | down | 0.0000371 | 0.000344064 | BQ133748 | 412 | 123 | 137 | 89 | 3e−68 | transport |
| 5367 | 3.197478079 | down | 0.0000218 | 0.000237664 | BF071366 | 475 | 68 | 162 | 41 | 3e−25 | |
| 5368 | 3.196810794 | down | 0.00000663 | 0.00010746 | BM309759 | 599 | 93 | 185 | 50 | 2e−50 | |
| 6369 | 3.19640409 | down | 0.000203279 | 0.001141304 | CA819374 | 491 | 54 | 75 | 72 | 3e−26 | |
| 5370 | 3.19573462 | down | 0.00000325 | 0.0000691 | AW309582 | 280 | 11 | 35 | 31 | 0.49 | |
| 5371 | 3.194446007 | down | 0.0000221 | 0.000240801 | BM954467 | 371 | 8 | 25 | 32 | 8.5 | |
| 5372 | 3.192068498 | down | 0.000278099 | 0.001434314 | AW350573 | 427 | 23 | 32 | 71 | 2e−08 | pathogenesis |
| 5373 | 3.191898669 | down | 0.00000729 | 0.000114115 | AW395344 | 427 | 10 | 19 | 52 | 1.8 | two-component signal transduction |
| | | | | | | | | | | | system (phosphorelay) |
| 5374 | 3.191421577 | down | 0.000000785 | 0.0000291 | BM092432 | 301 | 14 | 43 | 32 | 3.2 | |
| 5375 | 3.191005294 | down | 0.00025759 | 0.001358142 | BQ299017 | 362 | 12 | 34 | 35 | 0.92 | proteolysis and peptidolysis |
| 5376 | 3.190896226 | down | 0.000118691 | 0.000778048 | BQ629122 | 412 | 35 | 41 | 85 | 2e−16 | response to auxin stimulus |
| 5377 | 3.190365194 | down | 0.000385572 | 0.001819963 | BM567831 | 577 | 69 | 93 | 74 | 1e−36 | proline biosynthesis |
| 5378 | 3.190343038 | down | 0.000468085 | 0.002093773 | BI968172 | 793 | 28 | 40 | 70 | 2e−07 | regulation of transcription\, DNA- |
| | | | | | | | | | | | dependent |
| 5379 | 3.189348746 | down | 0.00000137 | 0.0000399 | CD392104 | 690 | 74 | 132 | 56 | 9e−33 | regulation of transcription\, DNA- |
| | | | | | | | | | | | dependent |
| 5380 | 3.18846328 | down | 0.0000123 | 0.000161231 | BE347480 | 576 | 60 | 98 | 61 | 3e−32 | carbohydrate metabolism |
| 5381 | 3.188331353 | down | 0.00000347 | 0.0000722 | BU544153 | 597 | 144 | 211 | 68 | 2e−72 | |
| 5382 | 3.187727221 | down | 0.0000108 | 0.000147697 | BQ080595 | 285 | 47 | 64 | 73 | 4e−22 | |
| 5383 | 3.18683 | down | 0.0000888 | 0.000633587 | BU765614 | 597 | 92 | 192 | 47 | 2e−40 | protein amino acid phosphorylation |
| 5384 | 3.185009936 | down | 0.0000604 | 0.000482145 | BE807315 | 680 | 150 | 206 | 72 | 5e−78 | |
| 5385 | 3.184373778 | down | 0.000000906 | 0.0000318 | BM094626 | 469 | 15 | 42 | 35 | 6.6 | |
| 5386 | 3.18172908 | down | 0.000000694 | 0.0000273 | BU544528 | 538 | 77 | 117 | 65 | 2e−42 | D-ribose metabolism |
| 5387 | 3.179957298 | down | 0.00000333 | 0.0000703 | BQ273031 | 539 | 41 | 113 | 36 | 1e−14 | metabolism |
| 5388 | 3.178470076 | down | 0.001079554 | 0.003854634 | CF809098 | 543 | 54 | 174 | 31 | 5e−09 | |
| 5389 | 3.178384505 | down | 0.00000321 | 0.0000686 | BQ081229 | 465 | 88 | 148 | 59 | 6e−49 | |
| 5390 | 3.177170265 | down | 0.00000142 | 0.0000407 | AW733701 | 551 | 163 | 182 | 89 | 2e−92 | |
| 5391 | 3.175150386 | down | 0.000000604 | 0.0000249 | CD408963 | 663 | 72 | 122 | 59 | 6e−37 | |
| 5392 | 3.174746846 | down | 0.00000515 | 0.0000928 | AW733720 | 475 | 73 | 150 | 48 | 6e−31 | |
| 5393 | 3.17454525 | down | 0.00000121 | 0.0000369 | AW597576 | 440 | 17 | 32 | 53 | 0.001 | |
| 5394 | 3.17430757 | down | 0.0000168 | 0.000199627 | AW164766 | 258 | 29 | 49 | 59 | 2e−12 | |
| 5395 | 3.17403573 | down | 0.000149561 | 0.000914433 | BE583643 | 431 | 16 | 46 | 34 | 0.65 | |
| 5396 | 3.281268281 | down | 0.00000407 | 0.0000795 | U43839.1 | | | | | | GmCK2p |
| 5397 | 3.281082067 | down | 0.0000133 | 0.000170757 | BQ299569 | 576 | 92 | 192 | 47 | 2e−33 | |
| 5398 | 3.27967223 | down | 0.00000286 | 0.0000637 | BI969917 | 428 | 38 | 73 | 52 | 2e−15 | metabolism |
| 5399 | 3.279583396 | down | 0.000000496 | 0.0000221 | BI969633 | 595 | 40 | 141 | 28 | 1e−10 | |
| 5400 | 3.27943697 | down | 0.000000298 | 0.0000161 | BI970254 | 778 | 42 | 69 | 60 | 6e−20 | |
| 5401 | 3.276186278 | down | 0.0000046 | 0.000086 | BI316593 | 534 | 20 | 27 | 74 | 9e−07 | proteolysis and peptidolysis |
| 5402 | 3.275466694 | down | 0.000000325 | 0.0000171 | BQ453416 | 426 | 82 | 136 | 60 | 6e−44 | potassium ion transport |
| 5403 | 3.275392237 | down | 0.00000115 | 0.0000361 | AW460238 | 350 | 8 | 17 | 47 | 0.84 | |
| 5404 | 3.161281254 | down | 0.0000284 | 0.000286697 | CD404180 | 302 | 8 | 11 | 72 | 9.4 | electron transport |
| 5405 | 3.161149375 | down | 0.0000538 | 0.000444808 | AW394524 | 400 | 12 | 39 | 30 | 2.6 | metabolism |
| 5406 | 3.161096405 | down | 0.000000582 | 0.0000243 | BQ453039 | 386 | 40 | 97 | 41 | 6e−10 | Heat shock transcription factor 31, |
| | | | | | | | | | | | regulation of transcription\, DNA- |
| | | | | | | | | | | | dependent |
| 5407 | 3.159304864 | down | 0.000167523 | 0.000994196 | AF243183.1 | | | | | | beta-ketoacyl-ACP synthetase I-2 |
| 5408 | 3.158808621 | down | 0.001047458 | 0.003770971 | BM522600 | 418 | 17 | 30 | 56 | 1e−04 | oligopeptide transport |
| 5409 | 3.158237068 | down | 0.0000208 | 0.000230533 | BM522804 | 437 | 80 | 122 | 65 | 3e−42 | |
| 5410 | 3.157984163 | down | 0.00000252 | 0.0000592 | AW350472 | 651 | 54 | 107 | 50 | 5e−28 | |
| 5411 | 3.157638016 | down | 0.0000108 | 0.00014756 | CA853090 | 510 | 89 | 164 | 54 | 5e−52 | carbohydrate metabolism |
| 5412 | 3.274855271 | down | 0.0000385 | 0.000352804 | CF809003 | 704 | 153 | 198 | 77 | 3e−95 | FAD2-2 microsomal omega-6 |
| | | | | | | | | | | | desaturase |
| 5413 | 3.274084377 | down | 0.000182171 | 0.001053535 | BI699402 | 422 | 12 | 30 | 40 | 5.2 | protein amino acid phosphorylation |
| 5414 | 3.27381791 | down | 0.000000912 | 0.0000318 | CD406943 | 535 | 29 | 35 | 82 | 6e−12 | protein amino acid phosphorylation |
| 5415 | 3.270800611 | down | 0.00000269 | 0.0000614 | AW310187 | 611 | 52 | 140 | 37 | 3e−17 | carbohydrate metabolism |
| 5416 | 3.269637921 | down | 0.000288954 | 0.001476926 | BM523175 | 487 | 107 | 140 | 76 | 8e−62 | proteolysis and peptidolysis |
| 5417 | 3.268748471 | down | 0.00000583 | 0.0000996 | CD408867 | 562 | 42 | 93 | 45 | 3e−14 | |
| 5418 | 3.267026548 | down | 0.000232427 | 0.001259548 | BE806891 | | | | | | |
| 5419 | 3.265887014 | down | 0.00000115 | 0.0000361 | AF243374.1 | | | | | | glutathione S-transferase GST 19 |
| 5420 | 3.264601963 | down | 0.00000113 | 0.0000358 | BI423792 | 251 | 9 | 20 | 45 | 7.3 | intracellular signaling cascade |
| 5421 | 3.264196469 | down | 0.00000414 | 0.0000804 | BE211509 | 428 | 50 | 61 | 81 | 2e−27 | |
| 5422 | 3.263845595 | down | 0.002634206 | 0.007267768 | CA852006 | 273 | 13 | 34 | 38 | 4.2 | carbohydrate metabolism |
| 5423 | 3.26369211 | down | 0.000000804 | 0.0000295 | CD391904 | 479 | 58 | 82 | 70 | 1e−29 | |
| 5424 | 3.263385041 | down | 0.00000979 | 0.000138062 | BM308587 | 521 | 88 | 104 | 84 | 3e−47 | mRNA splicing |
| 5425 | 3.262494635 | down | 0.00000471 | 0.0000873 | AI442808 | 547 | 109 | 140 | 77 | 2e−62 | |
| 5426 | 3.261874684 | down | 0.0000416 | 0.000372904 | BI968685 | 792 | 63 | 155 | 40 | 1e−20 | auxin mediated signaling |
| 5427 | 3.261729525 | down | 0.00000141 | 0.0000406 | CD410553 | 476 | 52 | 98 | 53 | 6e−25 | galactose metabolism |
| 5428 | 3.261652707 | down | 0.000000735 | 0.0000283 | BG238230 | 482 | 28 | 108 | 25 | 0.007 | |
| 5429 | 3.260746322 | down | 0.00000484 | 0.0000888 | CF808041 | 674 | 105 | 159 | 66 | 6e−55 | |
| 5430 | 3.260569823 | down | 0.00000139 | 0.0000403 | CD413673 | 400 | 16 | 45 | 35 | 0.69 | cation transport |
| 5431 | 3.258686029 | down | 0.0000253 | 0.000264979 | BE824396 | 666 | 77 | 113 | 68 | 3e−40 | proteolysis and peptidolysis |
| 5432 | 3.258114401 | down | 0.000000492 | 0.000022 | BG238134 | 542 | 14 | 39 | 35 | 1.8 | |
| 5433 | 3.256955466 | down | 0.000000378 | 0.0000188 | AJ563366.1 | | | | | | monosaccharide transporter |
| 5434 | 3.256674999 | down | 0.0000567 | 0.000462565 | CF807847 | 775 | 206 | 261 | 78 | e−118 | gluconeogenesis |
| 5435 | 3.256514826 | down | 0.0000358 | 0.000336872 | AW348090 | 655 | 32 | 62 | 51 | 1e−06 | |
| 5436 | 3.255936333 | down | 0.000000456 | 0.0000211 | CD415526 | 609 | 12 | 32 | 37 | 4.9 | |
| 5437 | 3.255344236 | down | 0.00000798 | 0.000120479 | BG725921 | 510 | 80 | 147 | 54 | 5e−38 | metabolism |
| 5438 | 3.254964865 | down | 0.000297702 | 0.001509945 | BQ629660 | 604 | 16 | 31 | 51 | 0.018 | |
| 5439 | 3.254836285 | down | 0.00000168 | 0.0000452 | BQ080718 | 557 | 119 | 185 | 64 | 1e−63 | response to oxidative stress |
| 5440 | 3.253750759 | down | 0.000276323 | 0.001427695 | BE806891 | | | | | | |
| 5441 | 3.253212257 | down | 0.0000149 | 0.000185308 | BM271403 | 546 | 71 | 185 | 38 | 1e−24 | |
| 5442 | 3.252264234 | down | 0.0000532 | 0.000441945 | CD395511 | 584 | 10 | 22 | 45 | 0.24 | |
| 5443 | 3.250551282 | down | 0.00000787 | 0.000119427 | CD391529 | 565 | 30 | 71 | 42 | 3e−08 | DNA replication initiation |
| 5444 | 3.250050512 | down | 0.00000191 | 0.0000496 | AW509184 | 282 | 13 | 31 | 41 | 9.2 | |
| 5445 | 3.249447487 | down | 0.002005196 | 0.005993902 | BI973527 | 566 | 132 | 185 | 71 | 2e−72 | proteolysis and peptidolysis |
| 5446 | 3.249089893 | down | 0.00000771 | 0.000117799 | AF353620.1 | | | | | | glutamine synthetase precursor |
| 5447 | 3.246331727 | down | 0.00000686 | 0.000109774 | AI966408 | 265 | 13 | 28 | 46 | 2.5 | |
| 5448 | 3.246198887 | down | 0.00000205 | 0.0000517 | AW309584 | 648 | 98 | 158 | 62 | 4e−55 | protein transport |
| 5449 | 3.244326256 | down | 0.0000144 | 0.000180904 | BQ296712 | 591 | 158 | 197 | 80 | 7e−91 | |
| 5450 | 3.244237651 | down | 0.0000038 | 0.0000763 | BE819903 | 598 | 42 | 95 | 44 | 8e−13 | |
| 5451 | 3.244129651 | down | 0.0000161 | 0.000194761 | BG154903 | 488 | 50 | 152 | 32 | 8e−23 | cellulose biosynthesis |
| 5452 | 3.243669356 | down | 0.0000318 | 0.00031075 | BM732034 | 542 | 57 | 75 | 76 | 2e−26 | |
| 5453 | 3.243263432 | down | 0.0000158 | 0.00019208 | BU547495 | 658 | 18 | 26 | 69 | 2e−05 | |
| 5454 | 3.241624084 | down | 0.00000765 | 0.000117474 | BU547411 | 618 | 75 | 109 | 68 | 7e−37 | defense response |
| 5455 | 3.241391934 | down | 0.00000578 | 0.000099 | BE657833 | | | | | | |
| 5456 | 3.240902858 | down | 0.000135162 | 0.000852731 | AW349964 | 729 | 104 | 205 | 50 | 7e−58 | proteolysis and peptidolysis |
| 5457 | 3.240111683 | down | 0.000946544 | 0.003511865 | CD392087 | 558 | 12 | 25 | 48 | 4.2 | |
| 5458 | 3.239837136 | down | 0.00000729 | 0.000114115 | BG316414 | 500 | 50 | 77 | 64 | 1e−19 | |
| 5459 | 3.239597362 | down | 0.0000166 | 0.000198056 | CK605704 | 431 | 18 | 62 | 29 | 3.2 | protein catabolism |
| 5460 | 3.238922375 | down | 0.000106672 | 0.0007192 | AW351287 | 725 | 58 | 158 | 36 | 3e−18 | leaf morphogenesis |
| 5461 | 3.236672624 | down | 0.0000184 | 0.000211968 | BM091603 | 215 | 14 | 34 | 41 | 3.2 | |
| 5462 | 3.236493491 | down | 0.00000394 | 0.000078 | BE348171 | 543 | 136 | 182 | 74 | 1e−76 | protein biosynthesis |
| 5463 | 3.234493488 | down | 0.00000923 | 0.000132424 | AF516880.1 | | | | | | expansin |
| 5464 | 3.233935014 | down | 0.0000151 | 0.000186831 | BQ610756 | 526 | 42 | 76 | 55 | 2e−13 | |
| 5465 | 3.232698299 | down | 0.0000128 | 0.000165816 | BE191387 | 635 | 106 | 202 | 52 | 1e−50 | |
| 5466 | 3.232312753 | down | 0.0000153 | 0.000188598 | BI787878 | 497 | 51 | 67 | 76 | 1e−25 | |
| 5467 | 3.23128802 | down | 0.0000849 | 0.000613176 | BU083922 | 528 | 93 | 133 | 69 | 3e−47 | protein amino acid phosphorylation |
| 5468 | 3.230962432 | down | 0.0000504 | 0.000425071 | AI973878 | 308 | 15 | 35 | 42 | 1.9 | |
| 5469 | 3.230852561 | down | 0.00000038 | 0.0000189 | BI943752 | 433 | 16 | 25 | 64 | 0.001 | |
| 5470 | 3.230550517 | down | 0.000480836 | 0.002137735 | AW706721 | 171 | 12 | 30 | 40 | 1.5 | |
| 5471 | 3.229617825 | down | 0.0000405 | 0.000365512 | CD391917 | 563 | 13 | 44 | 29 | 2.5 | |
| 5472 | 3.229468996 | down | 0.000164803 | 0.000981307 | BM094391 | 527 | 31 | 127 | 24 | 0.002 | |
| 5473 | 3.228937481 | down | 0.000106183 | 0.000716794 | AW507640 | 608 | 107 | 205 | 52 | 1e−52 | electron transport |
| 5474 | 3.228266058 | down | 0.00017382 | 0.001021889 | BI699402 | 422 | 12 | 30 | 40 | 5.2 | protein amino acid phosphorylation |
| 5475 | 3.226280344 | down | 0.00000143 | 0.0000408 | BE806262 | 569 | 94 | 181 | 51 | 1e−47 | transport |
| 5476 | 3.224699646 | down | 0.0000464 | 0.000402925 | BG509363 | 519 | 121 | 172 | 70 | 9e−63 | proteolysis and peptidolysis |
| 5477 | 3.223259252 | down | 0.0000715 | 0.000543337 | CF920420 | 629 | 82 | 156 | 52 | 7e−42 | |
| 5478 | 3.223122841 | down | 0.00000397 | 0.0000783 | BE821039 | 551 | 13 | 36 | 36 | 6.9 | |
| 5479 | 3.221542054 | down | 0.00000256 | 0.0000597 | AW101682 | 417 | 34 | 60 | 56 | 5e−17 | |
| 5480 | 3.220942164 | down | 0.00000213 | 0.000053 | BM527366 | 519 | 41 | 62 | 66 | 9e−23 | G-protein coupled receptor protein |
| | | | | | | | | | | | signaling pathway |
| 5481 | 3.220919806 | down | 0.00000715 | 0.000112845 | CD391200 | 365 | 31 | 34 | 91 | 1e−12 | protein amino acid phosphorylation |
| 5482 | 3.220741584 | down | 0.000699101 | 0.002802734 | BE819998 | 827 | 72 | 160 | 45 | 1e−24 | |
| 5483 | 3.220475314 | down | 0.00000303 | 0.0000663 | AI416659 | 489 | 105 | 127 | 82 | 7e−62 | carbohydrate biosynthesis |
| 5484 | 3.218560618 | down | 0.00000101 | 0.0000338 | AW100789 | 420 | 15 | 45 | 33 | 1.3 | |
| 5485 | 3.217504647 | down | 0.0000516 | 0.000432157 | BU544832 | 585 | 13 | 39 | 33 | 1.6 | |
| 5486 | 3.216943182 | down | 0.000133236 | 0.0008454 | BE823826 | 579 | 38 | 64 | 59 | 6e−15 | cation transport |
| 5487 | 3.216084886 | down | 0.000236495 | 0.001275529 | BQ741550 | 565 | 50 | 147 | 34 | 1e−14 | translational termination |
| 5488 | 3.215964395 | down | 0.001048044 | 0.003772198 | CD404298 | 504 | 55 | 110 | 50 | 2e−20 | |
| 5489 | 3.21508846 | down | 0.000000701 | 0.0000274 | BI425988 | 574 | 82 | 162 | 50 | 2e−39 | |
| 5490 | 3.213554815 | down | 0.0001395 | 0.000871656 | BG238184 | 474 | 97 | 121 | 80 | 3e−52 | transport |
| 5491 | 3.212804858 | down | 0.000024 | 0.00025494 | AI437929 | 403 | 91 | 128 | 71 | 1e−48 | transport |
| 5492 | 3.212576032 | down | 0.000000994 | 0.0000336 | BI942098 | 656 | 47 | 86 | 54 | 8e−18 | 4-coumarate:Coenzyme A ligase |
| | | | | | | | | | | | isoenzyme 4, phenylpropanoid |
| | | | | | | | | | | | metabolism |
| 5493 | 3.212392852 | down | 0.000000158 | 0.0000112 | BI424889 | 242 | 9 | 19 | 47 | 4.1 | regulation of transcription\, DNA- |
| | | | | | | | | | | | dependent |
| 5494 | 3.211972873 | down | 0.000760974 | 0.002982128 | CF808717 | 577 | 97 | 191 | 50 | 3e−51 | carbohydrate metabolism |
| 5495 | 3.211741993 | down | 0.000000998 | 0.0000336 | BU551158 | 601 | 86 | 168 | 51 | 2e−38 | |
| 5496 | 3.210003596 | down | 0.0000541 | 0.000446957 | BI945894 | 471 | 143 | 157 | 91 | 5e−77 | |
| 5497 | 3.209650118 | down | 0.0000185 | 0.00021253 | AI939079 | | | | | | |
| 5498 | 3.207733543 | down | 0.0000207 | 0.000229643 | BI317446 | 506 | 49 | 107 | 45 | 2e−18 | regulation of transcription |
| 5499 | 3.207538952 | down | 0.00000126 | 0.0000376 | CF806737 | 713 | 104 | 168 | 61 | 4e−53 | |
| 5500 | 3.207346075 | down | 0.000174667 | 0.001024122 | AW350642 | 734 | 72 | 139 | 51 | 5e−40 | electron transport |
| 5501 | 3.207171761 | down | 0.00000601 | 0.000101579 | BE823350 | 508 | 111 | 130 | 85 | 2e−60 | |
| 5502 | 3.20713282 | down | 0.000000933 | 0.0000324 | CD396407 | 398 | 14 | 38 | 36 | 0.31 | |
| 5503 | 3.207030047 | down | 0.000948396 | 0.003517003 | BU544977 | 539 | 15 | 30 | 50 | 0.011 | |
| 5504 | 3.206823242 | down | 0.0000171 | 0.000201703 | BQ253153 | 495 | 49 | 49 | 100 | 2e−23 | |
| 5505 | 3.206180388 | down | 0.00000863 | 0.000126508 | BU083490 | 531 | 18 | 33 | 54 | 0.008 | proteolysis and peptidolysis |
| 5506 | 3.205868918 | down | 0.0000254 | 0.000266052 | CF806066 | 656 | 161 | 216 | 74 | 7e−99 | cell elongation |
| 5507 | 3.205153819 | down | 0.00000164 | 0.0000445 | CD417005 | 537 | 29 | 30 | 96 | 5e−10 | cell differentiation |
| 5508 | 3.204653083 | down | 0.000000452 | 0.000021 | AF002258.1 | | | | | | 4-coumarate:CoA ligase isoenzyme 3 /// |
| | | | | | | | | | | | 4-coumarate:Coenzyme A ligase |
| | | | | | | | | | | | isoenzyme 4 |
| 5509 | 3.203870299 | down | 0.00000372 | 0.0000753 | BM108270 | 713 | 138 | 170 | 81 | 2e−70 | isoprenoid biosynthesis |
| 5510 | 3.202693871 | down | 0.000162016 | 0.000970703 | CF807049 | 606 | 134 | 166 | 80 | 3e−79 | |
| 5511 | 3.173764858 | down | 0.000000999 | 0.0000336 | AW348452 | 733 | 48 | 81 | 59 | 8e−22 | glycolysis |
| 5512 | 3.171805875 | down | 0.0000147 | 0.000182858 | BU579396 | 564 | 20 | 47 | 42 | 5e−04 | |
| 5513 | 3.171677391 | down | 0.00000479 | 0.0000881 | BE658824 | 362 | 27 | 70 | 38 | 0.001 | |
| 5514 | 3.171393705 | down | 0.00000312 | 0.0000676 | BU760714 | 445 | 83 | 113 | 73 | 5e−39 | transport |
| 5515 | 3.17097911 | down | 0.00000215 | 0.0000534 | AW307488 | 556 | 38 | 76 | 50 | 3e−14 | |
| 5516 | 3.17057081 | down | 0.000025 | 0.000263044 | BE820650 | 328 | 12 | 30 | 40 | 5.4 | metabolism |
| 5517 | 3.168922198 | down | 0.00000485 | 0.0000889 | AW348858 | 714 | 99 | 112 | 88 | 4e−55 | |
| 5518 | 3.16822446 | down | 0.00000151 | 0.0000424 | AW309927 | 666 | 85 | 149 | 57 | 2e−42 | |
| 5519 | 3.167387028 | down | 0.00039817 | 0.001861857 | BI969369 | 701 | 92 | 173 | 53 | 4e−52 | |
| 5520 | 3.167138233 | down | 0.000000198 | 0.0000128 | AW703886 | 468 | 14 | 22 | 63 | 0.032 | |
| 5521 | 3.166514522 | down | 0.0000403 | 0.000364077 | CD396444 | 645 | 64 | 154 | 41 | 3e−27 | |
| 5522 | 3.164590968 | down | 0.0000304 | 0.00030097 | BG839744 | 479 | 93 | 149 | 62 | 2e−50 | tricarboxylic acid cycle |
| 5523 | 3.162896011 | down | 0.000497421 | 0.002190607 | CD392921 | 576 | 40 | 119 | 33 | 2e−10 | regulation of transcription |
| 5524 | 3.162768528 | down | 0.00055566 | 0.002379393 | AW099083 | 377 | 29 | 40 | 72 | 5e−16 | carbohydrate biosynthesis |
| 5525 | 3.162617194 | down | 0.000000243 | 0.0000144 | CD396677 | 626 | 28 | 52 | 53 | 9e−10 | |
| 5526 | 3.162228262 | down | 0.0000427 | 0.000380566 | BG653913 | 509 | 101 | 153 | 66 | 2e−59 | |
| 5527 | 3.157563617 | down | 0.0000246 | 0.000259972 | BE659815 | 619 | 53 | 101 | 52 | 4e−26 | |
| 5528 | 3.156884196 | down | 0.00000167 | 0.0000451 | BI497613 | 570 | 88 | 179 | 49 | 2e−48 | metabolism |
| 5529 | 3.155948965 | down | 0.0000209 | 0.000230993 | BI892899 | 523 | 59 | 133 | 44 | 8e−22 | fatty acid beta-oxidation |
| 5530 | 3.155915769 | down | 0.00000144 | 0.0000411 | BG551038 | 475 | 64 | 129 | 49 | 6e−30 | regulation of transcription |
| 5531 | 3.155882757 | down | 0.0000395 | 0.00035901 | AF069738.1 | | | | | | Symbiotic ammonium transporter |
| | | | | | | | | | | | (SAT1) |
| 5532 | 3.155782509 | down | 0.00000344 | 0.0000718 | BG404916 | 410 | 15 | 60 | 25 | 2.9 | |
| 5533 | 3.155488043 | down | 0.00000686 | 0.000109781 | AW569282 | | | | | | |
| 5534 | 3.153517811 | down | 0.0000163 | 0.000196399 | CF807678 | 580 | 97 | 188 | 51 | 2e−45 | |
| 5535 | 3.152963657 | down | 0.00000251 | 0.0000591 | AW348149 | 805 | 196 | 239 | 82 | e−110 | gluconeogenesis |
| 5536 | 3.011487225 | down | 0.0000139 | 0.00017564 | CD417859 | 619 | 87 | 92 | 94 | 2e−47 | microtubule-based process |
| 5537 | 3.010841766 | down | 0.0000117 | 0.000155972 | BQ628737 | 212 | 15 | 40 | 37 | 7.2 | |
| 5538 | 3.010540473 | down | 0.000135068 | 0.000852568 | BM568150 | 574 | 117 | 175 | 66 | 1e−61 | |
| 5539 | 3.010164443 | down | 0.0000606 | 0.000483695 | CA783056 | 719 | 50 | 133 | 37 | 2e−10 | regulation of transcription\, DNA- |
| | | | | | | | | | | | dependent |
| 5540 | 3.008851817 | down | 0.00000627 | 0.000104593 | AW706989 | 518 | 111 | 171 | 64 | 1e−58 | |
| 5541 | 3.008823994 | down | 0.000052 | 0.000434281 | BM307262 | 592 | 106 | 127 | 83 | 1e−62 | |
| 5542 | 3.00856279 | down | 0.00000254 | 0.0000595 | BU549419 | 454 | 33 | 60 | 55 | 1e−17 | |
| 5543 | 3.00633245 | down | 0.00000128 | 0.0000382 | BG405067 | 501 | 52 | 160 | 32 | 7e−11 | negative regulation of flavonoid |
| | | | | | | | | | | | biosynthesis |
| 5544 | 3.00606805 | down | 0.000659864 | 0.002690236 | BQ785938 | 424 | 60 | 71 | 84 | 7e−29 | transport |
| 5545 | 3.00377849 | down | 0.00000237 | 0.000057 | BQ253787 | 329 | 9 | 28 | 32 | 3.3 | |
| 5546 | 3.003132908 | down | 0.000305337 | 0.001537491 | BQ612378 | 266 | 16 | 43 | 37 | 1.4 | |
| 5547 | 3.002245492 | down | 0.00000199 | 0.0000509 | CD404536 | 612 | 65 | 138 | 47 | 8e−22 | |
| 5548 | 3.001395807 | down | 0.0000268 | 0.000275505 | CA800336 | 520 | 55 | 167 | 32 | 2e−15 | sodium ion transport |
| 5549 | 3.000589507 | down | 0.0000216 | 0.000236375 | BE822985 | 660 | 25 | 67 | 37 | 4e−06 | protein amino acid phosphorylation |
| 5550 | 3.000233137 | down | 0.00000117 | 0.0000363 | AW396307 | 265 | 14 | 14 | 100 | 0.22 | cellulose biosynthesis |
| 5551 | 2.999447794 | down | 0.0000535 | 0.000443318 | AW508844 | 550 | 135 | 187 | 72 | 2e−76 | histidine biosynthesis |
| 5552 | 2.99890338 | down | 0.00000427 | 0.0000823 | CF807454 | 700 | 197 | 232 | 84 | e−118 | Ferredoxin-dependent glutamate |
| | | | | | | | | | | | synthase (glu), glutamate biosynthesis |
| 5553 | 2.998731655 | down | 0.0000318 | 0.00031075 | BG359427 | 374 | 42 | 93 | 45 | 8e−17 | |
| 5554 | 2.998618473 | down | 0.000000489 | 0.000022 | CF807080 | 589 | 114 | 174 | 65 | 7e−68 | alternative oxidase 2b |
| 5555 | 2.998316937 | down | 0.0000043 | 0.0000827 | BU548524 | 597 | 63 | 83 | 75 | 7e−31 | |
| 5556 | 2.996153208 | down | 0.0000019 | 0.0000493 | BI972260 | 329 | 14 | 27 | 51 | 0.66 | |
| 5557 | 2.995926429 | down | 0.000000835 | 0.0000303 | BI942916 | 353 | 64 | 78 | 82 | 8e−31 | DNA methylation |
| 5558 | 2.995553433 | down | 0.00000462 | 0.0000861 | BE822474 | 609 | 76 | 111 | 68 | 2e−42 | |
| 5559 | 2.994851504 | down | 0.00000201 | 0.0000511 | CA819741 | 329 | 13 | 47 | 27 | 3.3 | proton transport |
| 5560 | 2.993370875 | down | 0.00000104 | 0.0000341 | BI970945 | 787 | 32 | 52 | 61 | 2e−08 | |
| 5561 | 2.992477314 | down | 0.0000429 | 0.000381516 | BE823098 | 595 | 116 | 117 | 99 | 9e−60 | nucleosome assembly |
| 5562 | 2.991186048 | down | 0.00000245 | 0.0000581 | BU091393 | 449 | 49 | 102 | 48 | 2e−19 | transport |
| 5563 | 2.990344583 | down | 0.0000276 | 0.000280947 | BG509533 | 301 | 13 | 21 | 61 | 1.9 | cell wall modification |
| 5564 | 2.989570617 | down | 0.00000431 | 0.0000827 | BE657914 | 289 | 21 | 50 | 42 | 0.002 | |
| 5565 | 2.989481522 | down | 0.0000602 | 0.00048128 | BE059910 | 437 | 13 | 42 | 30 | 7.4 | |
| 5566 | 2.98870741 | down | 0.0000438 | 0.000387438 | BE820523 | 706 | 98 | 133 | 73 | 1e−51 | transport |
| 5567 | 2.988366407 | down | 0.0000087 | 0.000127276 | BE658030 | 449 | 49 | 76 | 64 | 3e−29 | |
| 5568 | 2.988014851 | down | 0.0000153 | 0.000188674 | CA799637 | 667 | 80 | 162 | 49 | 1e−42 | |
| 5569 | 2.987878382 | down | 0.00116065 | 0.004049035 | BI972022 | 543 | 85 | 186 | 45 | 6e−43 | |
| 5570 | 2.987837706 | down | 0.00011209 | 0.000745431 | CD414885 | 609 | 32 | 54 | 59 | 4e−13 | |
| 5571 | 3.15161633 | down | 0.0000166 | 0.000198056 | CA784580 | 681 | 104 | 120 | 86 | 2e−55 | galactose metabolism |
| 5572 | 3.151043903 | down | 0.00000319 | 0.0000684 | BQ080116 | 420 | 86 | 144 | 59 | 3e−37 | |
| 5573 | 3.150957721 | down | 0.0000057 | 0.0000984 | AW707170 | 420 | 71 | 140 | 50 | 3e−36 | protein amino acid phosphorylation |
| 5574 | 3.150866208 | down | 0.00000339 | 0.000071 | CA800793 | 306 | 12 | 30 | 40 | 5.5 | |
| 5575 | 3.148438764 | down | 0.00000282 | 0.000063 | BI785606 | 558 | 74 | 88 | 54 | 7e−38 | arginine biosynthesis |
| 5575 | 3.146736038 | down | 0.00000873 | 0.000127448 | AW508892 | 516 | 95 | 160 | 59 | 7e−57 | |
| 5577 | 3.146321038 | down | 0.000121885 | 0.00079387 | CF806817 | 661 | 52 | 81 | 64 | 3e−21 | |
| 5578 | 3.145853207 | down | 0.00000386 | 0.0000771 | BF598914 | 695 | 125 | 231 | 54 | 4e−61 | |
| 5579 | 3.145476804 | down | 0.0000267 | 0.000274684 | AW759870 | 515 | 99 | 160 | 61 | 2e−44 | |
| 5580 | 3.144580206 | down | 0.000812669 | 0.00313238 | BU763682 | | | | | | |
| 5581 | 3.144191989 | down | 0.0000027 | 0.0000616 | BI317201 | 495 | 11 | 20 | 55 | 1.5 | aromatic compound metabolism |
| 5582 | 3.14352366 | down | 0.00000084 | 0.0000304 | AW317569 | 359 | 37 | 44 | 84 | 4e−16 | |
| 5583 | 3.143445317 | down | 0.0000197 | 0.000221682 | AW397976 | 539 | 140 | 163 | 85 | 7e−80 | metabolism |
| 5584 | 3.141490102 | down | 0.00000471 | 0.0000873 | CD390602 | 407 | 22 | 47 | 46 | 1e−07 | |
| 5585 | 3.141022276 | down | 0.00000108 | 0.0000349 | BE803396 | 427 | 39 | 67 | 58 | 3e−16 | response to oxidative stress |
| 5586 | 3.140996767 | down | 0.0000188 | 0.000214992 | BQ741578 | 585 | 121 | 186 | 65 | 5e−63 | |
| 5587 | 3.138703554 | down | 0.00000762 | 0.000117192 | CF807143 | 570 | 131 | 186 | 70 | 2e−71 | metabolism |
| 5588 | 3.138665956 | down | 0.0000117 | 0.000155659 | BG042981 | 444 | 69 | 149 | 46 | 2e−29 | regulation of transcription |
| 5589 | 3.13811995 | down | 0.00000102 | 0.0000339 | BI969297 | 762 | 22 | 25 | 88 | 2e−05 | protein amino acid phosphorylation |
| 5590 | 3.137333167 | down | 0.000146579 | 0.000902065 | AI938916 | 342 | 15 | 43 | 34 | 9.4 | protein amino acid phosphorylation |
| 5591 | 3.136902194 | down | 0.00000382 | 0.0000765 | AW306644 | 499 | 49 | 85 | 57 | 6e−19 | |
| 5592 | 3.136252434 | down | 0.00000162 | 0.0000442 | BE822809 | 539 | 75 | 93 | 80 | 2e−40 | ‘de novo’ pyrimidine base biosynthesis |
| 5593 | 3.13542159 | down | 0.00031427 | 0.001571306 | BU550113 | 603 | 48 | 85 | 56 | 1e−20 | |
| 5594 | 3.134310895 | down | 0.000000954 | 0.0000327 | BM525106 | 571 | 82 | 149 | 55 | 7e−38 | proteolysis and peptidolysis |
| 5595 | 3.133205411 | down | 0.0000099 | 0.00013935 | AW396684 | 622 | 49 | 173 | 28 | 3e−12 | fatty acid biosynthesis |
| 5596 | 3.130469732 | down | 0.00000816 | 0.000122128 | BG509922 | | | | | | |
| 5597 | 3.12958918 | down | 0.0000053 | 0.0000942 | CD412359 | 627 | 64 | 106 | 60 | 8e−35 | |
| 5598 | 3.128573069 | down | 0.0000226 | 0.000244505 | BI971268 | 537 | 27 | 50 | 54 | 7e−07 | |
| 5599 | 3.127971629 | down | 0.00000227 | 0.0000554 | CF806812 | 560 | 46 | 78 | 58 | 1e−22 | carbohydrate metabolism |
| 5600 | 3.127402562 | down | 0.00000886 | 0.000128818 | BM177734 | 546 | 134 | 164 | 81 | 5e−82 | sterol biosynthesis |
| 5601 | 3.127381619 | down | 0.0000776 | 0.000574293 | AW350110 | 675 | 88 | 167 | 52 | 7e−38 | regulation of transcription |
| 5602 | 3.126553113 | down | 0.00000727 | 0.000114049 | BU762479 | 579 | 154 | 191 | 60 | 1e−84 | protein amino acid phosphorylation |
| 5603 | 3.12628583 | down | 0.000276454 | 0.001427978 | BQ628605 | 406 | 28 | 38 | 73 | 5e−11 | |
| 5604 | 3.125787657 | down | 0.0000014 | 0.0000404 | BU763658 | 420 | 28 | 29 | 96 | 6e−11 | regulation of transcription |
| 5605 | 3.125190223 | down | 0.000144137 | 0.000892063 | AI441027 | 220 | 17 | 59 | 28 | 1.9 | (G. max) hydroproline-rich protein, |
| | | | | | | | | | | | protein amino acid phosphorylation |
| 5606 | 3.125138121 | down | 0.00000754 | 0.000116381 | BM307069 | 403 | 19 | 26 | 73 | 0.11 | protein amino acid phosphorylation |
| 5607 | 3.124601912 | down | 0.000023 | 0.000247637 | AW186292 | 409 | 14 | 33 | 42 | 1.6 | |
| 5608 | 3.123956955 | down | 0.000000797 | 0.0000294 | CF805830 | 345 | 10 | 22 | 45 | 0.020 | transport |
| 5609 | 3.123932366 | down | 0.00000107 | 0.0000348 | CD413396 | 406 | 38 | 73 | 52 | 3e−13 | |
| 5610 | 3.123776853 | down | 0.0000317 | 0.000309412 | AW350457 | 540 | 51 | 134 | 38 | 7e−18 | |
| 5611 | 3.122308451 | down | 0.0000135 | 0.000172988 | BU544016 | 528 | 15 | 44 | 34 | 3.8 | |
| 5612 | 3.122164581 | down | 0.00000758 | 0.000116943 | BE821681 | 672 | 37 | 91 | 40 | 1e−07 | UDP-glucose metabolism |
| 5613 | 3.119603764 | down | 0.0000174 | 0.000204348 | CA935724 | 558 | 81 | 116 | 69 | 2e−39 | N-terminal protein myristoylation |
| 5614 | 3.118263663 | down | 0.000000637 | 0.0000257 | BU548356 | 617 | 82 | 121 | 67 | 7e−42 | transport |
| 5615 | 3.118164474 | down | 0.000000934 | 0.0000324 | AW349777 | 609 | 101 | 148 | 68 | 8e−53 | |
| 5616 | 3.117818354 | down | 0.00000106 | 0.0000344 | CF808738 | 570 | 103 | 151 | 68 | 2e−53 | |
| 5617 | 3.1161476 | down | 0.0000208 | 0.000230551 | BQ629429 | 575 | 121 | 153 | 79 | 1e−74 | carbohydrate biosynthesis |
| 5618 | 3.115543677 | down | 0.00000588 | 0.000100049 | BU763539 | 496 | 46 | 46 | 100 | 8e−20 | nucleosome assembly |
| 5619 | 3.114906468 | down | 0.0000339 | 0.000324326 | CD395831 | 668 | 75 | 91 | 82 | 3e−38 | |
| 5620 | 3.11417687 | down | 0.00000148 | 0.0000417 | BI424410 | 598 | 140 | 198 | 70 | 4e−83 | |
| 5621 | 3.113954784 | down | 0.0000705 | 0.000538729 | BI784526 | 515 | 105 | 157 | 66 | 2e−64 | |
| 5622 | 3.113620096 | down | 0.0000463 | 0.000402315 | BI967624 | 759 | 100 | 227 | 44 | 9e−32 | |
| 5623 | 3.113175177 | down | 0.000220872 | 0.001213006 | AW309945 | 676 | 105 | 158 | 66 | 2e−64 | L-aspartate transport |
| 5624 | 3.113127374 | down | 0.000114381 | 0.00075652 | BI699187 | 359 | 22 | 50 | 44 | 1e−04 | metabolism |
| 5625 | 3.112067611 | down | 0.0000138 | 0.000174871 | CD402114 | 642 | 58 | 109 | 53 | 2e−27 | |
| 5626 | 3.110568602 | down | 0.00000387 | 0.0000772 | BM085427 | 562 | 110 | 168 | 65 | 5e−63 | proteolysis and peptidolysis |
| 5627 | 3.110075675 | down | 0.000000346 | 0.0000177 | BI969422 | 549 | 29 | 69 | 42 | 2e−07 | |
| 5628 | 3.108746921 | down | 0.000357998 | 0.001722181 | AW307477 | 584 | 153 | 192 | 79 | 8e−85 | |
| 5629 | 3.107947141 | down | 0.00000117 | 0.0000363 | BE823652 | 758 | 80 | 205 | 39 | 3e−33 | apoptosis |
| 5630 | 3.107235085 | down | 0.0000008 | 0.0000294 | BM271340 | 429 | 86 | 113 | 76 | 1e−50 | |
| 5631 | 3.10709245 | down | 0.00000162 | 0.0000442 | BE611291 | 411 | 28 | 90 | 31 | 2e−06 | protein amino acid phosphorylation |
| 5632 | 3.104998276 | down | 0.000315683 | 0.001575861 | BI941740 | 796 | 188 | 259 | 72 | e−102 | galactose metabolism |
| 5633 | 3.104631328 | down | 0.002418612 | 0.006838583 | BQ080393 | 347 | 101 | 115 | 87 | 6e−55 | DNA repair |
| 5634 | 3.103476275 | down | 0.00000386 | 0.0000771 | AI735891 | 554 | 122 | 184 | 66 | 5e−70 | |
| 5635 | 3.101803988 | down | 0.00000702 | 0.000111293 | BQ094655 | 603 | 38 | 56 | 67 | 6e−16 | |
| 5636 | 3.10169221 | down | 0.00000784 | 0.000119185 | AW567674 | 226 | 10 | 21 | 47 | 7.2 | |
| 5637 | 3.100445519 | down | 0.000000944 | 0.0000325 | BQ079515 | 555 | 87 | 158 | 55 | 5e−43 | ciliary/flagellar motility |
| 5638 | 3.09936335 | down | 0.0000135 | 0.000172433 | BE021154 | 451 | 83 | 148 | 56 | 3e−47 | galactose metabolism |
| 5639 | 3.099193134 | down | 0.0000064 | 0.000105663 | BI892899 | 523 | 59 | 133 | 44 | 8e−22 | fatty acid beta-oxidation |
| 5640 | 3.098261638 | down | 0.00000375 | 0.0000756 | AW596527 | 566 | 123 | 177 | 69 | 3e−70 | |
| 5641 | 3.098067539 | down | 0.000365239 | 0.00174785 | AI442963 | 448 | 26 | 77 | 33 | 0.037 | intracellular signaling cascade |
| 5642 | 3.097730822 | down | 0.0000133 | 0.000170303 | BU544388 | 506 | 80 | 100 | 80 | 2e−49 | |
| 5643 | 3.097662143 | down | 0.00000807 | 0.000121248 | BG650176 | 384 | 14 | 48 | 29 | 1.8 | |
| 5644 | 3.096141789 | down | 0.0000354 | 0.000333289 | CD391838 | 573 | 10 | 30 | 33 | 7.5 | regulation of transcription\, DNA- |
| | | | | | | | | | | | dependent |
| 5645 | 3.095885442 | down | 0.000071 | 0.000541154 | AI939079 | | | | | | |
| 5646 | 3.093825559 | down | 0.0000187 | 0.000213694 | BQ613064 | 604 | 31 | 34 | 91 | 6e−11 | |
| 5647 | 3.093418837 | down | 0.00000131 | 0.0000385 | BI970635 | 791 | 110 | 130 | 84 | 3e−64 | electron transport |
| 5648 | 3.093049997 | down | 0.0000954 | 0.000667003 | BM269818 | 489 | 78 | 103 | 75 | 6e−42 | protein biosynthesis |
| 5649 | 3.092428146 | down | 0.00000383 | 0.0000767 | BU926677 | 469 | 97 | 158 | 61 | 5e−53 | |
| 5650 | 3.091983723 | down | 0.0000237 | 0.000252889 | BG239463 | 425 | 43 | 54 | 79 | 5e−19 | |
| 5651 | 3.091685532 | down | 0.0000133 | 0.000170634 | AW310367 | 435 | 21 | 64 | 32 | 0.001 | response to light |
| 5652 | 3.091520366 | down | 0.000000622 | 0.0000253 | AW598213 | 316 | 14 | 35 | 40 | 0.099 | |
| 5653 | 3.090482415 | down | 0.0000334 | 0.000321177 | BI320883 | 532 | 154 | 177 | 87 | 1e−88 | |
| 5654 | 3.088861172 | down | 0.000012 | 0.000158814 | AW156706 | 377 | 14 | 29 | 48 | 0.071 | |
| 5655 | 3.088553379 | down | 0.0000548 | 0.000450526 | BM954327 | 480 | 22 | 36 | 61 | 4e−06 | electron transport |
| 5656 | 3.087671572 | down | 0.00000261 | 0.0000604 | CA800068 | 740 | 118 | 211 | 55 | 8e−62 | |
| 5657 | 3.08635468 | down | 0.00000102 | 0.0000339 | BG044230 | 528 | 34 | 99 | 34 | 5e−08 | |
| 5658 | 3.086292082 | down | 0.0000016 | 0.0000439 | AW234029 | 357 | 57 | 104 | 54 | 3e−29 | |
| 5659 | 3.084913627 | down | 0.00000298 | 0.0000657 | BG508815 | 308 | 42 | 67 | 62 | 4e−20 | DNA methylation |
| 5660 | 3.084336855 | down | 0.00000701 | 0.000111253 | BI700424 | 421 | 19 | 25 | 76 | 5e−05 | electron transport |
| 5661 | 3.083963987 | down | 0.00000496 | 0.0000905 | AW397797 | 421 | 22 | 59 | 37 | 0.78 | regulation of carbohydrate metabolism |
| 5662 | 3.083719149 | down | 0.0000159 | 0.000192933 | AW350584 | 331 | 39 | 49 | 79 | 2e−20 | response to pathogenic fungi |
| 5663 | 3.083287263 | down | 0.00000173 | 0.0000462 | BF009987 | 612 | 20 | 64 | 31 | 0.024 | |
| 5664 | 3.082777556 | down | 0.0000449 | 0.000393419 | AW351319 | 728 | 75 | 140 | 53 | 6e−41 | lipid metabolism |
| 5665 | 3.081327233 | down | 0.000124732 | 0.000807872 | BI968454 | 418 | 13 | 33 | 39 | 1.0 | |
| 5666 | 3.080934621 | down | 0.0000011 | 0.0000351 | BU082628 | 506 | 42 | 66 | 63 | 2e−21 | chitin catabolism |
| 5667 | 3.079416767 | down | 0.0000936 | 0.000657118 | AF065435.1 | | | | | | nodulin |
| 5668 | 3.077912374 | down | 0.00000307 | 0.0000668 | BE822747 | 552 | 54 | 99 | 54 | 2e−25 | iron ion homeostasis |
| 5669 | 3.076575561 | down | 0.000013 | 0.000168352 | BM527497 | 569 | 53 | 92 | 57 | 4e−25 | protein amino acid phosphorylation |
| 5670 | 3.075558113 | down | 0.00000256 | 0.0000597 | CD405808 | 684 | 67 | 180 | 37 | 5e−23 | regulation of transcription |
| 5671 | 3.075299537 | down | 0.00000437 | 0.0000834 | BI968056 | 676 | 42 | 120 | 35 | 2e−10 | DNA methylation |
| 5672 | 3.074881177 | down | 0.00000118 | 0.0000364 | BU549663 | 710 | 27 | 44 | 61 | 5e−08 | |
| 5673 | 3.074797143 | down | 0.0000194 | 0.00022012 | BU546367 | 391 | 23 | 30 | 76 | 4e−08 | |
| 5674 | 3.074232883 | down | 0.0000112 | 0.000151484 | BE821862 | 696 | 66 | 88 | 75 | 6e−37 | |
| 5675 | 3.073310106 | down | 0.00000183 | 0.000048 | CD396216 | 511 | 19 | 32 | 59 | 3e−04 | |
| 5676 | 3.072560692 | down | 0.0000014 | 0.0000404 | BQ134032 | 424 | 52 | 88 | 59 | 3e−28 | metabolism |
| 5677 | 3.072177565 | down | 0.00000295 | 0.0000652 | AI460409 | 350 | 17 | 48 | 35 | 7.1 | oligopeptide transport |
| 5678 | 3.071131418 | down | 0.000000439 | 0.0000206 | CD411820 | 593 | 63 | 99 | 63 | 2e−35 | |
| 5679 | 3.070947045 | down | 0.0000744 | 0.000558566 | BQ629275 | 314 | 12 | 40 | 30 | 3.2 | proteolysis and peptidolysis |
| 5680 | 3.070614746 | down | 0.0000674 | 0.000521447 | BU082623 | 567 | 73 | 185 | 39 | 5e−30 | regulation of transcription\, DNA- |
| | | | | | | | | | | | dependent |
| 5681 | 3.069658754 | down | 0.000000792 | 0.0000293 | CF807005 | 648 | 38 | 122 | 31 | 2e−07 | DNA recombination |
| 5682 | 3.069386301 | down | 0.0000052 | 0.0000931 | BM525861 | 504 | 108 | 159 | 67 | 4e−61 | electron transport |
| 5683 | 3.068671081 | down | 0.0000028 | 0.0000629 | CK605578 | 562 | 140 | 167 | 83 | 3e−77 | |
| 5684 | 3.068179786 | down | 0.00000298 | 0.0000657 | AW278212 | 379 | 16 | 36 | 44 | 0.042 | response to pathogenic bacteria |
| 5685 | 3.068023506 | down | 0.0000115 | 0.000154524 | BE806164 | 438 | 68 | 107 | 63 | 3e−35 | carbohydrate metabolism |
| 5686 | 3.066754982 | down | 0.000000326 | 0.0000171 | BU761346 | 395 | 23 | 59 | 38 | 5e−06 | |
| 5687 | 3.066328183 | down | 0.0000427 | 0.000380301 | BI699282 | 450 | 31 | 57 | 54 | 8e−11 | |
| 5688 | 3.065914717 | down | 0.00000156 | 0.0000433 | CD394564 | 346 | 25 | 31 | 80 | 8e−09 | regulation of mitotic cell cycle |
| 5689 | 3.06374937 | down | 0.0000149 | 0.000184469 | BI967887 | 353 | 9 | 24 | 37 | 5.6 | |
| 5690 | 3.063492569 | down | 0.0000394 | 0.000358219 | AW307363 | 586 | 96 | 189 | 50 | 6e−47 | proteolysis and peptidolysis |
| 5691 | 3.06303534 | down | 0.001260155 | 0.004299661 | BG839283 | 747 | 178 | 221 | 80 | 3e−94 | Phenylalanine ammonia-lyase |
| | | | | | | | | | | | [soybeans, mRNA, 1427 nt], |
| | | | | | | | | | | | phenylpropanoid biosynthesis |
| 5692 | 3.063020143 | down | 0.00000193 | 0.0000497 | BE209880 | 337 | 13 | 21 | 61 | 0.17 | |
| 5693 | 3.062350126 | down | 0.0000535 | 0.000443511 | AW433263 | 346 | 13 | 18 | 72 | 0.045 | carbohydrate metabolism |
| 5694 | 3.061464358 | down | 0.0000279 | 0.000282687 | AW831517 | 442 | 84 | 156 | 53 | 7e−35 | |
| 5695 | 3.06083767 | down | 0.000000701 | 0.0000274 | BE824094 | 711 | 116 | 157 | 73 | 1e−63 | protein amino acid phosphorylation |
| 5696 | 3.06078234 | down | 0.0000898 | 0.000638251 | CA819221 | 545 | 77 | 181 | 42 | 4e−29 | |
| 5697 | 3.059228775 | down | 0.000000592 | 0.0000246 | BU549324 | 511 | 40 | 79 | 50 | 2e−16 | |
| 5698 | 3.057872797 | down | 0.00000331 | 0.0000699 | CD418314 | 593 | 10 | 20 | 50 | 6.1 | |
| 5699 | 3.057815476 | down | 0.000000984 | 0.0000334 | BI967506 | 789 | 164 | 222 | 73 | 8e−97 | carbohydrate metabolism |
| 5700 | 3.057400513 | down | 0.00000143 | 0.0000409 | AW310289 | 643 | 62 | 67 | 92 | 3e−31 | galactose metabolism |
| 5701 | 3.056834808 | down | 0.00000104 | 0.0000341 | BQ628332 | 225 | 8 | 22 | 36 | 7.2 | |
| 5702 | 3.05653773 | down | 0.00000232 | 0.0000561 | CA936699 | 430 | 35 | 62 | 56 | 1e−14 | protein amino acid phosphorylation |
| 5703 | 3.054402675 | down | 0.0000128 | 0.000166297 | BQ612438 | 319 | 14 | 38 | 36 | 0.38 | proteolysis and peptidolysis |
| 5704 | 3.053565951 | down | 0.00000147 | 0.0000416 | BU550790 | 634 | 85 | 177 | 48 | 2e−38 | |
| 5705 | 3.053213461 | down | 0.0000253 | 0.000264968 | BM887530 | 561 | 28 | 69 | 40 | 3e−06 | electron transport |
| 5706 | 3.05319907 | down | 0.000000712 | 0.0000277 | BE806131 | 521 | 118 | 163 | 72 | 2e−68 | |
| 5707 | 3.051806736 | down | 0.000000643 | 0.0000259 | BQ629101 | 516 | 27 | 103 | 26 | 1e−04 | apoptosis |
| 5708 | 3.051220435 | down | 0.00000513 | 0.0000926 | BE191592 | 713 | 136 | 191 | 71 | 1e−68 | |
| 5709 | 3.051101724 | down | 0.002130657 | 0.006255676 | CF806916 | 712 | 203 | 233 | 87 | e−122 | response to cold |
| 5710 | 3.050650685 | down | 0.00000947 | 0.000134939 | BE474469 | 439 | 125 | 146 | 85 | 7e−73 | UDP-glucose metabolism |
| 5711 | 3.047695141 | down | 0.000480396 | 0.002136648 | BG238202 | 480 | 74 | 115 | 64 | 2e−31 | G1 phase of mitotic cell cycle |
| 5712 | 3.047572918 | down | 0.00000106 | 0.0000344 | AW705813 | 501 | 29 | 68 | 42 | 3e−06 | regulation of transcription\, DNA- |
| | | | | | | | | | | | dependent |
| 5713 | 3.047518924 | down | 0.0000448 | 0.000393017 | BI969469 | 747 | 38 | 86 | 44 | 6e−12 | electron transport |
| 5714 | 3.0464797 | down | 0.00000389 | 0.0000773 | AF195028.1 | | | | | | plasma membrane Ca2+-ATPase |
| 5715 | 3.046468824 | down | 0.000106621 | 0.000719104 | BF067810 | 450 | 103 | 112 | 91 | 8e−58 | |
| 5716 | 3.045714889 | down | 0.0000116 | 0.000155047 | BU925875 | 559 | 73 | 85 | 85 | 5e−36 | adenine salvage pathway |
| 5717 | 3.045527673 | down | 0.00000787 | 0.000119427 | BE806391 | 491 | 11 | 29 | 37 | 7.3 | electron transport |
| 5718 | 3.044367272 | down | 0.00000121 | 0.0000371 | CF805934 | 670 | 25 | 81 | 30 | 0.036 | |
| 5719 | 3.04419666 | down | 0.00000136 | 0.0000397 | CD399802 | 535 | 61 | 75 | 81 | 1e−29 | tricarboxylic acid cycle |
| 5720 | 3.044058138 | down | 0.00000632 | 0.000105 | BM309549 | 506 | 102 | 129 | 79 | 2e−58 | |
| 5721 | 3.043817173 | down | 0.0000232 | 0.000249423 | BI700110 | 439 | 10 | 24 | 41 | 7.4 | |
| 5722 | 3.043427491 | down | 0.0000174 | 0.000204576 | AW156448 | 291 | 14 | 26 | 53 | 6e−04 | |
| 5723 | 3.042119034 | down | 0.000017 | 0.00020162 | AF243182.1 | | | | | | beta-ketoacyl-ACP synthetase I /// beta- |
| | | | | | | | | | | | ketoacyl-ACP synthetase I-2 |
| 5724 | 3.039867526 | down | 0.000582688 | 0.002464742 | CA802645 | 285 | 12 | 39 | 30 | 0.83 | |
| 5725 | 3.039017919 | down | 0.00000686 | 0.000109781 | BE020257 | 443 | 60 | 83 | 72 | 2e−31 | cell wall catabolism |
| 5726 | 3.038855722 | down | 0.00023285 | 0.001261114 | BG405325 | 341 | 83 | 113 | 73 | 3e−44 | sulfate transport |
| 5727 | 3.038746183 | down | 0.000153245 | 0.000930601 | BG359691 | 535 | 100 | 174 | 57 | 1e−59 | |
| 5728 | 3.0382624 | down | 0.0000327 | 0.000316047 | BI893826 | 347 | 26 | 71 | 36 | 3e−04 | |
| 5729 | 3.037197217 | down | 0.00000472 | 0.0000874 | AW569585 | 450 | 31 | 57 | 54 | 4e−07 | cell growth and/or maintenance |
| 5730 | 3.037123927 | down | 0.0000474 | 0.000408217 | AI496432 | 445 | 65 | 139 | 46 | 5e−31 | |
| 5731 | 3.037021232 | down | 0.00000361 | 0.0000737 | BE608520 | 619 | 154 | 173 | 89 | 2e−86 | Vacuolar processing enzyme 2 (VPE2), |
| | | | | | | | | | | | protein catabolism |
| 5732 | 3.036329185 | down | 0.0000721 | 0.000546107 | BE820999 | 487 | 68 | 99 | 68 | 2e−37 | regulation of transcription\, DNA- |
| | | | | | | | | | | | dependent |
| 5733 | 3.034580837 | down | 0.0000102 | 0.000142234 | CF808985 | 520 | 53 | 133 | 39 | 5e−20 | protein amino acid phosphorylation |
| 5734 | 3.034010521 | down | 0.000000846 | 0.0000305 | BG042435 | 598 | 125 | 191 | 65 | 2e−66 | threonine catabolism |
| 5735 | 3.0333323 | down | 0.00000127 | 0.0000379 | AW351025 | 587 | 12 | 19 | 63 | 0.14 | |
| 5736 | 3.032987691 | down | 0.00000386 | 0.0000771 | CA802930 | 280 | 15 | 32 | 46 | 9.3 | |
| 5737 | 3.032718397 | down | 0.000263931 | 0.001380547 | BG551684 | 254 | 9 | 22 | 40 | 3.3 | regulation of transcription\, DNA- |
| | | | | | | | | | | | dependent |
| 5738 | 3.032069342 | down | 0.00000249 | 0.0000587 | CD398272 | 428 | 9 | 19 | 47 | 7.0 | |
| 5739 | 3.031207701 | down | 0.0000469 | 0.000405869 | CA784730 | 713 | 162 | 242 | 66 | 7e−82 | regulation of transcription\, DNA- |
| | | | | | | | | | | | dependent |
| 5740 | 3.03110388 | down | 0.00000681 | 0.000109267 | BI970186 | 768 | 83 | 123 | 67 | 5e−44 | carbohydrate metabolism |
| 5741 | 3.029382922 | down | 0.001888895 | 0.005749223 | CD412731 | 474 | 23 | 26 | 88 | 3e−07 | regulation of translation |
| 5742 | 3.028652878 | down | 0.0000109 | 0.000149195 | BM522736 | 543 | 28 | 87 | 32 | 3e−04 | |
| 5743 | 3.02846676 | down | 0.000000394 | 0.0000192 | CD392844 | 284 | 13 | 30 | 43 | 4.1 | |
| 5744 | 3.028316471 | down | 0.0000848 | 0.000612314 | BI968148 | 788 | 88 | 183 | 48 | 4e−40 | |
| 5745 | 3.028225886 | down | 0.001995276 | 0.005972967 | CA784647 | 727 | 38 | 98 | 38 | 2e−11 | |
| 5746 | 3.028153188 | down | 0.00000695 | 0.000110774 | BI969499 | 788 | 138 | 184 | 75 | 2e−82 | |
| 5747 | 3.028149453 | down | 0.0000916 | 0.000647935 | AW350857 | 633 | 86 | 166 | 51 | 2e−47 | carbohydrate metabolism |
| 5748 | 3.023975454 | down | 0.000006 | 0.000101444 | BE331541 | 583 | 93 | 130 | 71 | 8e−48 | |
| 5749 | 3.023642831 | down | 0.00000263 | 0.0000608 | CD413418 | 458 | 47 | 102 | 46 | 2e−22 | |
| 5750 | 3.023264346 | down | 0.0000356 | 0.000334672 | AW309595 | 666 | 49 | 95 | 51 | 6e−20 | |
| 5751 | 3.023027769 | down | 0.0000225 | 0.000243882 | CF807537 | 601 | 89 | 152 | 58 | 1e−41 | |
| 5752 | 3.01959612 | down | 0.0000221 | 0.000240863 | U64866.1 | | | | | | sali5-4a protein |
| 5753 | 3.018823972 | down | 0.0000339 | 0.000324429 | CD409023 | 614 | 74 | 150 | 49 | 3e−36 | |
| 5754 | 3.017829082 | down | 0.0000013 | 0.0000384 | BI497998 | 539 | 86 | 120 | 71 | 5e−48 | cell elongation |
| 5755 | 3.014952264 | down | 0.00000191 | 0.0000496 | BU545419 | 329 | 65 | 94 | 69 | 1e−36 | cell wall modification |
| 5756 | 3.014857736 | down | 0.00000151 | 0.0000424 | CD414601 | 555 | 11 | 28 | 39 | 9.1 | oligopeptide transport |
| 5757 | 3.013852955 | down | 0.0000114 | 0.000153515 | CD405202 | 692 | 93 | 144 | 64 | 8e−50 | |
| 5758 | 3.013782799 | down | 0.000421037 | 0.001939368 | BE021143 | 441 | 115 | 118 | 97 | 2e−65 | response to high light intensity |
| 5759 | 3.01341089 | down | 0.0000199 | 0.000223376 | BU763409 | 328 | 12 | 33 | 36 | 3.2 | |
| 5760 | 3.013171016 | down | 0.00000257 | 0.0000599 | BM522553 | 392 | 13 | 43 | 30 | 0.89 | mRNA processing |
| 5761 | 3.012418729 | down | 0.000194477 | 0.001104031 | CK606338 | 782 | 12 | 30 | 40 | 1.9 | MRNA from stress-induced gene (H4) |
| 5762 | 3.012341867 | down | 0.0000474 | 0.000408527 | CA799403 | 620 | 41 | 104 | 39 | 1e−17 | |
| 5763 | 3.012302952 | down | 0.0000571 | 0.000465414 | BM525748 | | | | | | |
| 5764 | 3.012172086 | down | 0.00000525 | 0.0000935 | CA800952 | 666 | 77 | 174 | 44 | 3e−35 | |
| 5765 | 3.0121375 | down | 0.00000227 | 0.0000554 | AW156348 | 346 | 18 | 59 | 30 | 1.9 | |
| 5766 | 3.012081991 | down | 0.00000557 | 0.0000969 | BU550056 | 614 | 53 | 141 | 37 | 5e−13 | |
| 5767 | 3.012052327 | down | 0.0000396 | 0.000359616 | BE822299 | 359 | 10 | 16 | 62 | 1.5 | apoptosis |
| 5768 | 3.012039061 | down | 0.0000146 | 0.000182135 | CA783780 | 546 | 75 | 172 | 43 | 1e−31 | |
| 5769 | 2.986392585 | down | 0.000045 | 0.000394511 | CF808863 | 759 | 135 | 160 | 84 | 3e−69 | |
| 5770 | 2.986315362 | down | 0.000472179 | 0.002107918 | BQ612772 | 174 | 12 | 37 | 32 | 5.5 | |
| 5771 | 2.985957822 | down | 0.000185299 | 0.001065975 | BI316229 | 498 | 97 | 136 | 71 | 1e−52 | |
| 5772 | 2.985621572 | down | 0.000206677 | 0.00115443 | BU547004 | 651 | 30 | 109 | 27 | 0.058 | |
| 5773 | 2.985332364 | down | 0.00000255 | 0.0000596 | CD415247 | 614 | 135 | 167 | 80 | 7e−76 | |
| 5774 | 2.984637463 | down | 0.0000643 | 0.000504382 | CA953418 | 576 | 95 | 152 | 62 | 7e−54 | |
| 5775 | 2.983344904 | down | 0.0000183 | 0.000210717 | BQ628554 | 298 | 23 | 82 | 28 | 0.50 | |
| 5776 | 2.98320618 | down | 0.000005 | 0.0000909 | CA936289 | 567 | 146 | 189 | 77 | 1e−80 | protein amino acid phosphorylation |
| 5777 | 2.981710853 | down | 0.00000118 | 0.0000363 | AI856147 | 723 | 120 | 200 | 60 | 3e−59 | isoprenoid biosynthesis |
| 5778 | 2.98112646 | down | 0.00000442 | 0.0000841 | AW704299 | 503 | 94 | 143 | 65 | 4e−45 | |
| 5779 | 2.980919466 | down | 0.0000347 | 0.000330144 | CD416142 | 296 | 20 | 22 | 90 | 7e−05 | protein biosynthesis |
| 5780 | 2.979353401 | down | 0.00000922 | 0.000132419 | BI785751 | 558 | 155 | 170 | 91 | 3e−86 | (G. max) mRNA for proliferating cell |
| | | | | | | | | | | | nuclear antigen (PCNA), partial, |
| | | | | | | | | | | | regulation of DNA replication |
| 5781 | 2.977740941 | down | 0.000000311 | 0.0000167 | CD409521 | 618 | 75 | 190 | 39 | 1e−30 | |
| 5782 | 2.977407514 | down | 0.000269345 | 0.001400326 | AW164694 | 437 | 86 | 137 | 62 | 2e−43 | carbohydrate metabolism |
| 5783 | 2.976149285 | down | 0.0000592 | 0.000476067 | AW433155 | 414 | 39 | 63 | 61 | 3e−16 | |
| 5784 | 2.753881011 | down | 0.0000164 | 0.000197074 | BU547416 | 663 | 89 | 152 | 58 | 6e−44 | |
| 5785 | 2.753875015 | down | 0.00000962 | 0.000136464 | BU081394 | 600 | 20 | 74 | 27 | 1.2 | exocytosis |
| 5786 | 2.753683036 | down | 0.000015 | 0.000185595 | AI495867 | 354 | 85 | 118 | 72 | 8e−52 | |
| 5787 | 2.75314888 | down | 0.0000188 | 0.000215218 | BQ630127 | 605 | 114 | 197 | 57 | 2e−61 | electron transport |
| 5788 | 2.752640221 | down | 0.000124436 | 0.00080669 | CA784540 | 766 | 169 | 259 | 65 | 1e−99 | |
| 5789 | 2.752020078 | down | 0.00000159 | 0.0000439 | BM092075 | 334 | 34 | 42 | 80 | 1e−16 | |
| 5790 | 2.749738689 | down | 0.0000178 | 0.000207573 | BE022719 | 308 | 52 | 100 | 52 | 2e−24 | electron transport |
| 5791 | 2.749592903 | down | 0.00000104 | 0.0000341 | AW568433 | 449 | 33 | 41 | 80 | 6e−14 | |
| 5792 | 2.749493928 | down | 0.00000263 | 0.0000608 | BG155554 | 375 | 22 | 50 | 44 | 3e−06 | |
| 5793 | 2.748437623 | down | 0.000330602 | 0.001625951 | BE474824 | 420 | 21 | 48 | 43 | 2e−06 | mitochondrial electron transport\, NADH |
| | | | | | | | | | | | to ubiquinone |
| 5794 | 2.748285736 | down | 0.00000157 | 0.0000434 | BM887472 | 551 | 100 | 140 | 71 | 2e−54 | mitogen-activated protein kinase 1 |
| 5795 | 2.745801256 | down | 0.0000041 | 0.00008 | BQ611581 | 220 | 14 | 46 | 30 | 7.2 | |
| 5796 | 2.745674657 | down | 0.000356057 | 0.001715258 | BU081882 | 570 | 40 | 84 | 47 | 2e−17 | |
| 5797 | 2.744519782 | down | 0.0000164 | 0.000197137 | BI971744 | 437 | 13 | 28 | 46 | 1.5 | |
| 5798 | 2.744365258 | down | 0.000061 | 0.000485903 | AW279450 | 464 | 125 | 156 | 80 | 5e−69 | regulation of transcription\, DNA- |
| | | | | | | | | | | | dependent |
| 5799 | 2.744283113 | down | 0.000321281 | 0.001593023 | BU549116 | | | | | | |
| 5800 | 2.743501581 | down | 0.000611981 | 0.002547202 | BI893750 | 413 | 29 | 78 | 37 | 5e−05 | |
| 5801 | 2.743285116 | down | 0.00000426 | 0.000082 | CD412154 | 661 | 17 | 39 | 43 | 0.17 | |
| 5802 | 2.743182622 | down | 0.0000053 | 0.0000942 | CF922570 | 670 | 88 | 102 | 86 | 4e−51 | |
| 5803 | 2.624805145 | down | 0.000000855 | 0.0000307 | BI967831 | 688 | 134 | 151 | 88 | 8e−72 | |
| 5804 | 2.624654802 | down | 0.000382585 | 0.001809022 | BF067691 | 509 | 99 | 152 | 65 | 4e−53 | regulation of transcription |
| 5805 | 2.624312958 | down | 0.0000016 | 0.0000439 | AW756063 | 447 | 88 | 146 | 60 | 2e−45 | |
| 5806 | 2.623549155 | down | 0.0000148 | 0.000184109 | CF806402 | 692 | 29 | 69 | 42 | 2e−07 | |
| 5807 | 2.623367283 | down | 0.00000768 | 0.000117626 | CD415130 | 674 | 82 | 158 | 51 | 2e−37 | regulation of transcription |
| 5808 | 2.622577032 | down | 0.00058872 | 0.002480023 | CF806194 | 580 | 152 | 178 | 85 | 1e−86 | |
| 5809 | 2.622399383 | down | 0.0000262 | 0.000271615 | BM271008 | 345 | 11 | 21 | 52 | 9.3 | |
| 5810 | 2.622034899 | down | 0.00000892 | 0.000129441 | CD416697 | 683 | 47 | 114 | 41 | 5e−16 | regulation of transcription\, DNA- |
| | | | | | | | | | | | dependent |
| 5811 | 2.621916016 | down | 0.00000396 | 0.0000783 | BE660328 | 738 | 161 | 173 | 93 | 2e−90 | small GTPase mediated signal |
| | | | | | | | | | | | transduction |
| 5812 | 2.621274674 | down | 0.000148952 | 0.00091234 | CA938402 | 428 | 16 | 32 | 50 | 0.075 | G-protein coupled receptor protein |
| | | | | | | | | | | | signaling pathway |
| 5813 | 2.620829727 | down | 0.00000257 | 0.0000599 | BE820632 | 481 | 24 | 43 | 55 | 7e−08 | metabolism |
| 5814 | 2.620822806 | down | 0.0000144 | 0.000180332 | BM178069 | 552 | 75 | 181 | 41 | 1e−33 | lipid metabolism |
| 5815 | 2.620565896 | down | 0.000693001 | 0.002783698 | CD413632 | 572 | 16 | 39 | 41 | 0.40 | |
| 5816 | 2.620272433 | down | 0.0000444 | 0.00039034 | AW423948 | 384 | 41 | 61 | 67 | 1e−18 | |
| 5817 | 2.619930352 | down | 0.00000279 | 0.0000627 | BG650766 | 461 | 78 | 117 | 66 | 2e−41 | |
| 5818 | 2.619900515 | down | 0.00000191 | 0.0000496 | CD416464 | 558 | 49 | 77 | 63 | 2e−24 | |
| 5819 | 2.619806242 | down | 0.00000126 | 0.0000376 | BE329937 | 454 | 80 | 92 | 86 | 8e−43 | |
| 5820 | 2.619399837 | down | 0.00000489 | 0.0000894 | AW351160 | 689 | 98 | 125 | 78 | 2e−57 | electron transport |
| 5821 | 2.619158318 | down | 0.000000785 | 0.0000291 | J03920.1 | | | | | | auxin-regulated protein (Aux22) |
| 5822 | 2.618811979 | down | 0.000275005 | 0.001423229 | BE824014 | 654 | 120 | 154 | 77 | 2e−57 | ossification |
| 5823 | 2.618537564 | down | 0.000002 | 0.0000509 | AW350843 | 597 | 45 | 76 | 59 | 9e−23 | |
| 5824 | 2.618532714 | down | 0.000416952 | 0.001925977 | BQ094164 | 550 | 81 | 130 | 62 | 8e−38 | tricarboxylic acid cycle |
| 5825 | 2.618085321 | down | 0.0000242 | 0.000256666 | BQ079384 | 522 | 86 | 178 | 48 | 6e−41 | electron transport |
| 5826 | 2.617928416 | down | 0.0000978 | 0.000677928 | CF807238 | 281 | 16 | 39 | 41 | 0.099 | |
| 5827 | 2.617792208 | down | 0.000491613 | 0.002171643 | BF597286 | 455 | 12 | 54 | 22 | 0.73 | |
| 5828 | 2.617237521 | down | 0.00000827 | 0.000123058 | AW310488 | 667 | 115 | 151 | 76 | 7e−67 | proteolysis and peptidolysis |
| 5829 | 2.616480395 | down | 0.00000288 | 0.000064 | BU551247 | 482 | 41 | 74 | 55 | 1e−14 | establishment of competence for |
| | | | | | | | | | | | transformation |
| 5830 | 2.616439613 | down | 0.0000109 | 0.000149019 | BE024043 | 450 | 130 | 151 | 86 | 6e−69 | protein amino acid phosphorylation |
| 5831 | 2.616379383 | down | 0.000011 | 0.000149798 | CA785274 | 675 | 56 | 78 | 11 | 3e−29 | |
| 5832 | 2.615798866 | down | 0.0000233 | 0.000250216 | BM092010 | 293 | 16 | 44 | 36 | 4.2 | protein amino acid phosphorylation |
| 5833 | 2.615153956 | down | 0.0000159 | 0.000193 | CD401381 | 395 | 64 | 81 | 79 | 2e−36 | cell wall modification |
| 5834 | 2.614660852 | down | 0.000312816 | 0.001564664 | BM885094 | 469 | 35 | 112 | 31 | 0.005 | regulation of transcription |
| 5835 | 2.614154953 | down | 0.0000106 | 0.000146275 | CA819086 | 441 | 44 | 108 | 40 | 9e−09 | translational termination |
| 5836 | 2.614059867 | down | 0.0000327 | 0.000316007 | BE346680 | 643 | 144 | 214 | 67 | 3e−83 | signal transduction |
| 5837 | 2.613803569 | down | 0.0001045 | 0.00070912 | U08384.1 | | | | | | BiP isoform A |
| 5838 | 2.975292932 | down | 0.000567777 | 0.002420017 | BF009919 | 296 | 28 | 68 | 41 | 0.001 | regulation of transcription |
| 5839 | 2.974098251 | down | 0.0000111 | 0.000151357 | AW349402 | 554 | 47 | 107 | 43 | 1e−14 | |
| 5840 | 2.973573968 | down | 0.0000457 | 0.000399128 | BQ298800 | 525 | 35 | 107 | 32 | 0.052 | |
| 5841 | 2.973552275 | down | 0.0000035 | 0.0000725 | AI938957 | 566 | 28 | 46 | 60 | 3e−11 | |
| 5842 | 2.973027556 | down | 0.000145442 | 0.000897533 | AW350291 | 698 | 129 | 175 | 73 | 5e−71 | |
| 5843 | 2.97251654 | down | 0.00000173 | 0.0000461 | CF806685 | 664 | 86 | 128 | 67 | 2e−46 | protein amino acid phosphorylation |
| 5844 | 2.971687659 | down | 0.000000969 | 0.000033 | BG725902 | 486 | 140 | 141 | 99 | 2e−76 | small GTPase mediated signal |
| | | | | | | | | | | | transduction |
| 5845 | 2.9716705 | down | 0.00000523 | 0.0000932 | BI699466 | 420 | 11 | 25 | 44 | 0.78 | regulation of transcription\, DNA- |
| | | | | | | | | | | | dependent |
| 5846 | 2.971327288 | down | 0.0000455 | 0.000397403 | BM176963 | 428 | 17 | 57 | 29 | 1.4 | oligopeptide transport |
| 5847 | 2.970618444 | down | 0.000173149 | 0.001018743 | BM527789 | 585 | 69 | 152 | 45 | 1e−29 | regulation of transcription |
| 5848 | 2.970487553 | down | 0.000556688 | 0.002383253 | CF805829 | 380 | 18 | 36 | 50 | 6e−06 | transport |
| 5849 | 2.969931354 | down | 0.0000161 | 0.000194761 | BG507403 | 551 | 51 | 115 | 44 | 5e−19 | |
| 5850 | 2.969196618 | down | 0.000061 | 0.000485903 | CD409217 | 289 | 11 | 23 | 47 | 4.1 | regulation of transcription\, DNA- |
| | | | | | | | | | | | dependent |
| 5851 | 2.968723151 | down | 0.00000669 | 0.000108231 | BF009486 | 416 | 77 | 98 | 78 | 3e−44 | carbohydrate metabolism |
| 5852 | 2.968378542 | down | 0.000047 | 0.000406543 | CF805696 | 542 | 91 | 176 | 51 | 1e−47 | |
| 5853 | 2.968250652 | down | 0.000396351 | 0.001856813 | BE021143 | 441 | 115 | 118 | 97 | 2e−65 | response to high light intensity |
| 5854 | 2.968218165 | down | 0.00000296 | 0.0000652 | CF806715 | 687 | 175 | 225 | 77 | e−100 | cell differentiation |
| 5855 | 2.968128757 | down | 0.000859031 | 0.003265142 | CA802422 | 245 | 9 | 21 | 42 | 5.4 | |
| 5856 | 2.968050377 | down | 0.000000331 | 0.0000172 | AW100760 | 328 | 25 | 48 | 52 | 2e−07 | |
| 5857 | 2.967830092 | down | 0.0000016 | 0.0000439 | BM085121 | 515 | 66 | 109 | 60 | 3e−31 | electron transport |
| 5858 | 2.967153548 | down | 0.0000139 | 0.000176052 | CF808509 | 554 | 138 | 184 | 75 | 9e−80 | protein amino acid phosphorylation |
| 5859 | 2.965426947 | down | 0.0000181 | 0.000209414 | AW310689 | 363 | 25 | 71 | 35 | 0.002 | |
| 5860 | 2.965402941 | down | 0.0000159 | 0.000192924 | BI427126 | 552 | 151 | 184 | 82 | 1e−85 | |
| 5861 | 2.964280946 | down | 0.0000118 | 0.000156584 | AW101778 | 344 | 20 | 62 | 32 | 3.2 | multidrug transport |
| 5862 | 2.964130564 | down | 0.00000644 | 0.000105803 | BQ630519 | 555 | 70 | 94 | 74 | 1e−37 | |
| 5863 | 2.963883822 | down | 0.00000109 | 0.000035 | BU546941 | 643 | 39 | 99 | 39 | 1e−12 | aging |
| 5864 | 2.963463635 | down | 0.00000144 | 0.0000411 | AW278270 | 337 | 39 | 54 | 72 | 1e−19 | |
| 5865 | 2.963411215 | down | 0.00000082 | 0.0000299 | AW348801 | 565 | 38 | 66 | 57 | 9e−14 | cytokinesis by cell plate formation |
| 5866 | 2.960752127 | down | 0.000000633 | 0.0000256 | AW350481 | 752 | 20 | 70 | 28 | 2.4 | |
| 5867 | 2.960315083 | down | 0.000217502 | 0.0011994 | BG789629 | 479 | 99 | 151 | 65 | 8e−57 | response to wounding |
| 5868 | 2.960226764 | down | 0.0000759 | 0.000566217 | CA801538 | 434 | 89 | 132 | 67 | 2e−44 | glycolysis |
| 5869 | 2.958051034 | down | 0.000000322 | 0.000017 | BM143260 | 420 | 21 | 53 | 39 | 3e−07 | protein amino acid phosphorylation |
| 5870 | 2.958033965 | down | 0.0000808 | 0.000592545 | BE659841 | 386 | 12 | 29 | 41 | 2.5 | |
| 5871 | 2.957578441 | down | 0.0000105 | 0.000144642 | BQ253736 | 420 | 104 | 142 | 73 | 6e−54 | regulation of transcription |
| 5872 | 2.957334261 | down | 0.0000106 | 0.000146441 | AW734306 | | | | | | |
| 5873 | 2.956709175 | down | 0.0000255 | 0.000266283 | BE022458 | 204 | 11 | 23 | 47 | 3.2 | transport |
| 5874 | 2.956632023 | down | 0.000353347 | 0.001705258 | CF807867 | 737 | 190 | 237 | 80 | e−117 | methionine metabolism |
| 5875 | 2.95650168 | down | 0.00000588 | 0.000100049 | CF809129 | 694 | 65 | 132 | 49 | 1e−27 | |
| 5876 | 2.955679227 | down | 0.000000876 | 0.0000311 | CD416275 | 354 | 29 | 38 | 76 | 3e−11 | |
| 5877 | 2.955345446 | down | 0.0000623 | 0.000493476 | BI971353 | 709 | 77 | 163 | 47 | 6e−38 | |
| 5878 | 2.955076457 | down | 0.0000353 | 0.000333289 | CD407874 | 700 | 134 | 154 | 87 | 7e−73 | adenine salvage pathway |
| 5879 | 2.953645682 | down | 0.00000375 | 0.0000755 | AW309856 | 461 | 37 | 46 | 80 | 1e−16 | |
| 5880 | 2.953107164 | down | 0.0000112 | 0.000151484 | BU926932 | 610 | 137 | 191 | 71 | 1e−81 | carbohydrate metabolism |
| 5881 | 2.953062161 | down | 0.00000345 | 0.0000718 | BU765120 | 598 | 46 | 73 | 63 | 3e−21 | metabolism |
| 5882 | 2.952968515 | down | 0.0000141 | 0.000177557 | BQ079740 | 407 | 8 | 26 | 30 | 8.2 | |
| 5883 | 2.952890817 | down | 0.00000369 | 0.0000751 | BI317193 | 506 | 81 | 147 | 55 | 4e−43 | mitochondrial translocation |
| 5884 | 2.952371334 | down | 0.000000873 | 0.000031 | BQ453397 | 467 | 51 | 72 | 70 | 5e−24 | response to light |
| 5885 | 2.952194287 | down | 0.00000128 | 0.0000382 | CD393322 | 563 | 53 | 71 | 74 | 3e−27 | |
| 5886 | 2.952109023 | down | 0.0000432 | 0.000383438 | BE020664 | 406 | 46 | 78 | 58 | 7e−20 | transport |
| 5887 | 2.949233603 | down | 0.000000955 | 0.0000327 | AW348176 | 552 | 44 | 65 | 67 | 6e−26 | G-protein coupled receptor protein |
| | | | | | | | | | | | signaling pathway |
| 5888 | 2.949199934 | down | 0.000390219 | 0.00183586 | BQ079388 | 530 | 60 | 137 | 43 | 2e−23 | nucleotide-sugar metabolism |
| 5889 | 2.946452451 | down | 0.00000808 | 0.000121312 | BQ610467 | 610 | 119 | 200 | 59 | 1e−62 | |
| 5890 | 2.94354372 | down | 0.00000213 | 0.0000531 | CD411120 | 705 | 11 | 28 | 39 | 2.1 | |
| 5891 | 2.94353334 | down | 0.00000518 | 0.0000929 | BE820316 | 645 | 125 | 146 | 85 | 1e−68 | metabolism |
| 5892 | 2.941007242 | down | 0.0000076 | 0.000117138 | BG043466 | 379 | 56 | 111 | 50 | 9e−26 | |
| 5893 | 2.940846556 | down | 0.0000694 | 0.000532466 | AB083026.1 | | | | | | syringolide-induced protein 14-1-1 |
| 5894 | 2.940833895 | down | 0.000100442 | 0.000689927 | BM732383 | 563 | 58 | 77 | 75 | 2e−27 | transport |
| 5895 | 2.940522397 | down | 0.0000137 | 0.000174694 | BE440295 | 635 | 66 | 163 | 40 | 1e−31 | electron transport |
| 5896 | 2.940269053 | down | 0.000000437 | 0.0000205 | AI416757 | 453 | 19 | 31 | 61 | 7e−04 | |
| 5897 | 2.939800377 | down | 0.000018 | 0.00020917 | BU765099 | 429 | 34 | 50 | 68 | 6e−15 | |
| 5898 | 2.939126057 | down | 0.00000819 | 0.000122286 | CA935282 | 596 | 123 | 188 | 65 | 1e−66 | regulation of nitrogen utilization |
| 5899 | 2.939081642 | down | 0.000000867 | 0.000031 | BF595946 | 429 | 88 | 108 | 81 | 5e−48 | protein amino acid phosphorylation |
| 5900 | 2.938956715 | down | 0.000000282 | 0.0000156 | BE058069 | 390 | 14 | 25 | 56 | 0.004 | proteolysis and peptidolysis |
| 5901 | 2.937764699 | down | 0.0000802 | 0.000589138 | CK605984 | 700 | 78 | 129 | 60 | 3e−31 | aromatic compound metabolism |
| 5902 | 2.936265025 | down | 0.000000649 | 0.000026 | U13987.1 | | | | | | Inducible nitrate reductase 2 (INR2) |
| 5903 | 2.936021862 | down | 0.001160488 | 0.004048845 | BM139971 | | | | | | |
| 5904 | 2.935864342 | down | 0.000595739 | 0.00250007 | AW396769 | 647 | 58 | 134 | 43 | 2e−24 | |
| 5905 | 2.935137534 | down | 0.0000129 | 0.000167666 | BM085107 | 546 | 118 | 181 | 65 | 2e−60 | potassium ion transport |
| 5906 | 2.935062662 | down | 0.00000408 | 0.0000796 | BQ740743 | 457 | 15 | 59 | 25 | 2.1 | |
| 5907 | 2.934848514 | down | 0.0000213 | 0.000234181 | BU547811 | | | | | | |
| 5908 | 2.933808455 | down | 0.000188851 | 0.001080372 | AW277978 | 653 | 152 | 180 | 84 | 2e−90 | response to ethylene stimulus |
| 5909 | 2.933260185 | down | 0.000171485 | 0.001011482 | BM523755 | 280 | 12 | 25 | 48 | 1.9 | L-serine biosynthesis |
| 5910 | 2.933187138 | down | 0.0000315 | 0.000308373 | AW307139 | 412 | 95 | 124 | 76 | 3e−52 | |
| 5911 | 2.932602793 | down | 0.00000552 | 0.0000964 | CA936143 | 559 | 63 | 132 | 47 | 6e−29 | electron transport |
| 5912 | 2.932262088 | down | 0.00000306 | 0.0000667 | BI968134 | 753 | 31 | 138 | 22 | 0.007 | DNA replication |
| 5913 | 2.932189607 | down | 0.00000303 | 0.0000663 | CD417955 | 537 | 18 | 58 | 31 | 0.46 | proteolysis and peptidolysis |
| 5914 | 2.931674399 | down | 0.00000376 | 0.0000757 | CF809244 | 533 | 19 | 56 | 33 | 0.054 | |
| 5915 | 2.931517655 | down | 0.00000135 | 0.0000394 | CD395152 | 682 | 58 | 119 | 48 | 5e−20 | |
| 5916 | 2.931455658 | down | 0.00000478 | 0.0000881 | CD398783 | 368 | 11 | 11 | 100 | 1.7 | transport |
| 5917 | 2.93127666 | down | 0.0000756 | 0.000564901 | BM307216 | 291 | 13 | 19 | 68 | 0.075 | proteolysis and peptidolysis |
| 5918 | 2.931059878 | down | 0.0000107 | 0.000146441 | BI973739 | 548 | 162 | 182 | 89 | 7e−98 | potassium ion transport |
| 5919 | 2.93082169 | down | 0.0000524 | 0.000436773 | CF808526 | 552 | 131 | 154 | 85 | 6e−73 | metabolism |
| 5920 | 2.929149245 | down | 0.0000192 | 0.000218401 | BI425175 | 308 | 14 | 54 | 25 | 1.9 | |
| 5921 | 2.927906509 | down | 0.00000162 | 0.0000441 | AI965868 | | | | | | |
| 5922 | 2.927774297 | down | 0.000645693 | 0.002650272 | AW309868 | 559 | 14 | 35 | 40 | 0.84 | protein amino acid phosphorylation |
| 5923 | 2.926227139 | down | 0.000114976 | 0.000759652 | CF808970 | 495 | 47 | 96 | 48 | 4e−16 | indole-3-acetic acid induced protein |
| | | | | | | | | | | | ARG-2 homolog, response to reactive |
| | | | | | | | | | | | oxygen species |
| 5924 | 2.925787549 | down | 0.00000854 | 0.000125533 | CF808992 | 548 | 114 | 170 | 67 | 4e−61 | Uricase (Nod-35), purine base |
| | | | | | | | | | | | metabolism |
| 5925 | 2.925576863 | down | 0.00000631 | 0.000104931 | BM271008 | 345 | 11 | 21 | 52 | 9.3 | |
| 5926 | 2.925266848 | down | 0.000000518 | 0.0000227 | BI969313 | 696 | 122 | 156 | 78 | 8e−66 | glycine catabolism |
| 5927 | 2.923737927 | down | 0.00000857 | 0.000125961 | AW507733 | 371 | 13 | 42 | 30 | 6.5 | cytoskeleton organization and |
| | | | | | | | | | | | biogenesis |
| 5928 | 2.922797175 | down | 0.00000142 | 0.0000406 | BU083717 | 447 | 16 | 48 | 33 | 7.7 | transcription |
| 5929 | 2.921601675 | down | 0.0000243 | 0.000257082 | BU763748 | 644 | 133 | 160 | 83 | 1e−78 | |
| 5930 | 2.920726524 | down | 0.0000105 | 0.000144605 | CF806056 | 653 | 57 | 151 | 37 | 4e−27 | |
| 5931 | 2.920640962 | down | 0.00000547 | 0.0000959 | AW310339 | 507 | 44 | 69 | 63 | 2e−20 | two-component signal transduction |
| | | | | | | | | | | | system (phosphorelay) |
| 5932 | 2.920323744 | down | 0.0000195 | 0.000221197 | BE822248 | 720 | 96 | 112 | 85 | 2e−52 | regulation of transcription\, DNA- |
| | | | | | | | | | | | dependent |
| 5933 | 2.918575968 | down | 0.0000573 | 0.000466046 | BM527789 | 585 | 69 | 152 | 45 | 1e−29 | regulation of transcription |
| 5934 | 2.918459174 | down | 0.001370041 | 0.004564973 | U08383.1 | | | | | | BiP isoform B |
| 5935 | 2.915115188 | down | 0.00000469 | 0.0000871 | CD393514 | 514 | 63 | 83 | 75 | 4e−31 | |
| 5936 | 2.91482093 | down | 0.00000348 | 0.0000723 | AW705560 | 444 | 35 | 40 | 87 | 2e−12 | Ubiquitin, ubiquitin-dependent protein |
| | | | | | | | | | | | catabolism |
| 5937 | 2.914662251 | down | 0.000000903 | 0.0000318 | AW164239 | | | | | | |
| 5938 | 2.914407523 | down | 0.0000107 | 0.000146768 | BF716244 | 389 | 97 | 123 | 78 | 3e−54 | cell wall modification |
| 5939 | 2.914007209 | down | 0.000146364 | 0.000901148 | BU765286 | 261 | 12 | 42 | 28 | 0.85 | |
| 5940 | 2.913936691 | down | 0.000000948 | 0.0000326 | BM954481 | 555 | 80 | 188 | 42 | 9e−32 | cytidine deamination |
| 5941 | 2.912498856 | down | 0.0000445 | 0.000391097 | BG511678 | | | | | | |
| 5942 | 2.910580877 | down | 0.000106674 | 0.0007192 | BQ298226 | | | | | | |
| 5943 | 2.910321181 | down | 0.00000138 | 0.00004 | BM731664 | 421 | 67 | 79 | 84 | 1e−36 | |
| 5944 | 2.910128098 | down | 0.0000123 | 0.000161801 | BG726532 | 487 | 106 | 122 | 86 | 1e−62 | |
| 5945 | 2.909992158 | down | 0.00000265 | 0.0000609 | BU550929 | 678 | 98 | 118 | 83 | 2e−49 | protein amino acid phosphorylation |
| 5946 | 2.909933833 | down | 0.00000258 | 0.0000599 | BG725767 | 455 | 9 | 48 | 18 | 2.8 | regulation of transcription |
| 5947 | 2.909635078 | down | 0.0000121 | 0.000159558 | AW202087 | 266 | 47 | 64 | 73 | 1e−21 | carbohydrate metabolism |
| 5948 | 2.909222376 | down | 0.00000232 | 0.0000561 | BE608448 | 625 | 90 | 193 | 46 | 1e−37 | fatty acid biosynthesis |
| 5949 | 2.908733674 | down | 0.00000604 | 0.000101853 | AW310165 | 468 | 61 | 96 | 63 | 4e−32 | |
| 5950 | 2.908717104 | down | 0.0000896 | 0.000637108 | AW760424 | 406 | 18 | 27 | 66 | 2e−04 | |
| 5951 | 2.907989429 | down | 0.00000733 | 0.000114471 | BG790288 | 367 | 23 | 57 | 40 | 0.95 | |
| 5952 | 2.90672625 | down | 0.00059211 | 0.002490397 | BI787704 | 593 | 88 | 154 | 57 | 2e−39 | |
| 5953 | 2.906680161 | down | 0.000220665 | 0.001212222 | BI785070 | 547 | 17 | 47 | 36 | 0.015 | apoptosis |
| 5954 | 2.905433937 | down | 0.0000193 | 0.000219281 | BM178271 | 563 | 73 | 96 | 76 | 6e−41 | |
| 5955 | 2.905346494 | down | 0.000000796 | 0.0000294 | AW306851 | 187 | 36 | 59 | 61 | 6e−17 | protein amino acid phosphorylation |
| 5956 | 2.905096363 | down | 0.00000355 | 0.000073 | CD403154 | 614 | 93 | 96 | 96 | 1e−46 | nucleosome assembly |
| 5957 | 2.904706828 | down | 0.00000821 | 0.00012241 | BI969048 | 706 | 44 | 70 | 62 | 1e−20 | two-component signal transduction |
| | | | | | | | | | | | system (phosphorelay) |
| 5958 | 2.90278074 | down | 0.00000102 | 0.0000339 | BQ080602 | 541 | 29 | 40 | 72 | 8e−12 | response to auxin stimulus |
| 5959 | 2.902689912 | down | 0.00000228 | 0.0000554 | BI893662 | 510 | 115 | 142 | 80 | 3e−65 | proteolysis and peptidolysis |
| 5960 | 2.902374156 | down | 0.000000687 | 0.0000271 | BE800599 | 529 | 38 | 176 | 21 | 6e−06 | proteolysis and peptidolysis |
| 5961 | 2.902112394 | down | 0.0000116 | 0.000155393 | BU549585 | 584 | 142 | 153 | 92 | 7e−83 | transport |
| 5962 | 2.901668201 | down | 0.001030515 | 0.003726019 | CA801096 | 609 | 149 | 175 | 85 | 1e−82 | acetyl-CoA biosynthesis |
| 5963 | 2.901166181 | down | 0.00000861 | 0.000126391 | CK605966 | 547 | 52 | 68 | 76 | 7e−24 | translational elongation |
| 5964 | 2.900881693 | down | 0.0000107 | 0.000146593 | AW350819 | 633 | 45 | 120 | 37 | 1e−18 | protein amino acid phosphorylation |
| 5965 | 2.900396064 | down | 0.00000475 | 0.0000878 | CA819035 | 574 | 60 | 141 | 42 | 2e−28 | nucleoside metabolism |
| 5966 | 2.900285975 | down | 0.00000113 | 0.0000357 | BI702227 | 488 | 19 | 67 | 28 | 2.5 | |
| 5967 | 2.899908174 | down | 0.00000373 | 0.0000753 | CD397245 | 295 | 15 | 46 | 32 | 2.5 | |
| 5968 | 2.899451973 | down | 0.000208057 | 0.001160243 | AW620767 | 582 | 98 | 182 | 53 | 6e−40 | gluconeogenesis |
| 5969 | 2.897830362 | down | 0.0000004 | 0.0000194 | AI736607 | 447 | 23 | 29 | 79 | 5e−06 | regulation of transcription\, DNA- |
| | | | | | | | | | | | dependent |
| 5970 | 2.897718092 | down | 0.00000745 | 0.000115833 | BU547416 | 663 | 89 | 152 | 58 | 6e−44 | |
| 5971 | 2.897522645 | down | 0.0000251 | 0.00026356 | BQ080142 | 499 | 52 | 133 | 39 | 3e−21 | protein amino acid phosphorylation |
| 5972 | 2.895969773 | down | 0.0000377 | 0.000348225 | AW317389 | 304 | 16 | 54 | 29 | 1.4 | carbohydrate metabolism |
| 5973 | 2.895656025 | down | 0.00000138 | 0.00004 | CF807823 | 746 | 18 | 48 | 37 | 0.16 | |
| 5974 | 2.895517468 | down | 0.0000963 | 0.000672397 | AI496413 | 447 | 53 | 102 | 51 | 2e−22 | |
| 5975 | 2.894980395 | down | 0.00000345 | 0.0000718 | BG238344 | 387 | 62 | 120 | 51 | 6e−31 | metabolism |
| 5976 | 2.893620133 | down | 0.000000678 | 0.0000269 | CD417522 | 598 | 46 | 72 | 63 | 3e−22 | |
| 5977 | 2.891548173 | down | 0.0000281 | 0.000284465 | BI969591 | 727 | 137 | 185 | 74 | 2e−77 | electron transport |
| 5978 | 2.891312803 | down | 0.0000452 | 0.000395201 | CF806186 | 568 | 174 | 189 | 92 | 3e−98 | selenocysteine incorporation |
| 5979 | 2.890493941 | down | 0.00000311 | 0.0000674 | BQ611482 | 612 | 89 | 189 | 47 | 4e−51 | electron transport |
| 5980 | 2.890191112 | down | 0.00000236 | 0.0000569 | BQ473785 | 567 | 33 | 68 | 48 | 3e−09 | |
| 5981 | 2.888482995 | down | 0.0000257 | 0.000267502 | BG362929 | 518 | 30 | 35 | 85 | 7e−12 | regulation of transcription\, DNA- |
| | | | | | | | | | | | dependent |
| 5982 | 2.887265285 | down | 0.00000267 | 0.0000612 | BQ612756 | 429 | 58 | 75 | 77 | 3e−28 | fatty acid biosynthesis |
| 5983 | 2.885517908 | down | 0.00000475 | 0.0000878 | CD412510 | 553 | 19 | 26 | 73 | 5e−04 | defense response |
| 5984 | 2.884268351 | down | 0.0000177 | 0.000206684 | U77678.1 | | | | | | asparagine synthetase 2 |
| 5985 | 2.884054335 | down | 0.00000633 | 0.000105028 | AW348613 | 748 | 130 | 165 | 78 | 2e−75 | |
| 5986 | 2.883964727 | down | 0.00000593 | 0.000100488 | CD390440 | 440 | 32 | 67 | 47 | 6e−10 | cell differentiation |
| 5987 | 2.883825359 | down | 0.0000829 | 0.000602797 | BI972234 | 542 | 106 | 171 | 61 | 8e−57 | |
| 5988 | 2.882986874 | down | 0.00000329 | 0.0000696 | BE821492 | 623 | 63 | 128 | 49 | 3e−31 | |
| 5989 | 2.88292102 | down | 0.0000425 | 0.000378895 | BG650945 | 517 | 89 | 176 | 50 | 5e−44 | electron transport |
| 5990 | 2.881563017 | down | 0.000013 | 0.000168048 | BU544158 | 496 | 47 | 70 | 67 | 8e−26 | |
| 5991 | 2.880660817 | down | 0.000039 | 0.000355433 | BU577698 | 569 | 75 | 180 | 41 | 2e−26 | salicylic acid mediated signaling |
| | | | | | | | | | | | pathway (systemic acquired resistance) |
| 5992 | 2.879385787 | down | 0.0000719 | 0.000545109 | AW348645 | 590 | 28 | 51 | 54 | 2e−06 | |
| 5993 | 2.879226298 | down | 0.00000571 | 0.0000985 | BE023364 | 405 | 16 | 44 | 36 | 0.95 | cytochrome biogenesis |
| 5994 | 2.879211444 | down | 0.00000244 | 0.0000579 | CA782572 | 568 | 140 | 155 | 90 | 8e−74 | secretory pathway |
| 5995 | 2.878847241 | down | 0.000018 | 0.000208483 | BE612223 | 545 | 40 | 71 | 56 | 4e−17 | |
| 5996 | 2.878795896 | down | 0.000023 | 0.000247487 | CD394310 | 483 | 91 | 107 | 85 | 2e−53 | proteolysis and peptidolysis |
| 5997 | 2.877709539 | down | 0.00000273 | 0.000062 | BE820570 | 697 | 65 | 118 | 55 | 1e−28 | |
| 5998 | 2.877212283 | down | 0.00000451 | 0.000085 | BU548498 | 482 | 61 | 148 | 41 | 1e−27 | protein amino acid phosphorylation |
| 5999 | 2.877048657 | down | 0.00000375 | 0.0000755 | BM954581 | 555 | 17 | 52 | 32 | 3.1 | |
| 6000 | 2.876689728 | down | 0.00000426 | 0.000082 | CF808912 | 556 | 147 | 168 | 87 | 9e−85 | response to light |
| 6001 | 2.876404403 | down | 0.00000661 | 0.000107329 | CF806170 | 704 | 97 | 171 | 56 | 3e−56 | |
| 6002 | 2.876029212 | down | 0.00000724 | 0.000113774 | BE657185 | 799 | 133 | 210 | 63 | 4e−72 | |
| 6003 | 2.875463639 | down | 0.000246218 | 0.001314026 | BQ611839 | 603 | 65 | 114 | 57 | 1e−25 | |
| 6004 | 2.875269066 | down | 0.00000103 | 0.0000339 | BG511169 | 284 | 17 | 58 | 29 | 1.1 | regulation of transcription\, DNA- |
| | | | | | | | | | | | dependent |
| 6005 | 2.874669496 | down | 0.00000104 | 0.0000341 | BG157338 | 463 | 70 | 94 | 74 | 7e−36 | aromatic compound metabolism |
| 6006 | 2.874235763 | down | 0.00000221 | 0.0000544 | CK605868 | 664 | 201 | 220 | 91 | e−116 | methionine biosynthesis |
| 6007 | 2.873568367 | down | 0.00000108 | 0.0000348 | AW317212 | 486 | 27 | 55 | 49 | 2e−10 | mitochondrial translocation |
| 6008 | 2.873552031 | down | 0.00000281 | 0.000063 | CD404338 | 666 | 37 | 95 | 38 | 2e−12 | |
| 6009 | 2.873020485 | down | 0.00000214 | 0.0000532 | AW598055 | 443 | 9 | 15 | 60 | 0.89 | |
| 6010 | 2.872620995 | down | 0.000000407 | 0.0000196 | BQ080756 | 557 | 62 | 97 | 63 | 3e−31 | |
| 6011 | 2.87156527 | down | 0.00000177 | 0.0000468 | CA938729 | 475 | 61 | 92 | 66 | 2e−33 | |
| 6012 | 2.870894009 | down | 0.0000195 | 0.000220698 | CA784474 | 743 | 78 | 165 | 47 | 1e−30 | regulation of transcription |
| 6013 | 2.870731583 | down | 0.00000239 | 0.0000573 | BE822970 | 223 | 14 | 41 | 34 | 0.38 | transport |
| 6014 | 2.870513385 | down | 0.000626654 | 0.002593063 | BM523757 | 415 | 23 | 36 | 63 | 4e−08 | |
| 6015 | 2.870338021 | down | 0.0000456 | 0.000398159 | BF009213 | 587 | 148 | 170 | 87 | 1e−87 | cell growth and/or maintenance |
| 6016 | 2.869110118 | down | 0.0000489 | 0.000416627 | CD396068 | 719 | 17 | 51 | 33 | 2.9 | |
| 6017 | 2.869076766 | down | 0.0000103 | 0.00014299 | BI970595 | 712 | 43 | 74 | 58 | 2e−19 | cytoskeleton organization and |
| | | | | | | | | | | | biogenesis |
| 6018 | 2.868577422 | down | 0.0000048 | 0.0000882 | CD405336 | 636 | 48 | 77 | 62 | 5e−25 | regulation of transcription |
| 6019 | 2.867689511 | down | 0.0000895 | 0.000636889 | BU549027 | 578 | 93 | 123 | 75 | 4e−49 | |
| 6020 | 2.865481665 | down | 0.00000122 | 0.0000371 | AI930780 | 561 | 77 | 176 | 43 | 9e−32 | regulation of transcription |
| 6021 | 2.865442434 | down | 0.0000872 | 0.000625509 | BF599103 | 403 | 56 | 123 | 45 | 3e−23 | protein amino acid phosphorylation |
| 6022 | 2.865183081 | down | 0.0000336 | 0.000322783 | BI967997 | 788 | 161 | 199 | 80 | 3e−89 | biological_process unknown |
| 6023 | 2.865163966 | down | 0.000000572 | 0.0000242 | AY383736.1 | | | | | | 3-ketoacyl-CoA thiolase |
| 6024 | 2.865041422 | down | 0.0000404 | 0.000364405 | BI970904 | 799 | 24 | 33 | 72 | 5e−07 | |
| 6025 | 2.864808561 | down | 0.0000133 | 0.000170303 | BE658601 | 475 | 18 | 34 | 52 | 0.004 | |
| 6026 | 2.863487072 | down | 0.000000942 | 0.0000325 | CD400920 | 391 | 12 | 24 | 50 | 0.30 | protein amino acid phosphorylation |
| 6027 | 2.863439788 | down | 0.000853834 | 0.003250948 | AW508123 | 459 | 43 | 73 | 58 | 3e−20 | metal ion transport |
| 6028 | 2.863362733 | down | 0.0000848 | 0.00061241 | BE801594 | 420 | 19 | 69 | 27 | 0.35 | |
| 6029 | 2.863246829 | down | 0.000002 | 0.0000509 | BU548417 | 470 | 68 | 126 | 53 | 2e−37 | |
| 6030 | 2.862847548 | down | 0.00000437 | 0.0000834 | BF071407 | 409 | 84 | 142 | 59 | 3e−45 | |
| 6031 | 2.862780078 | down | 0.000383639 | 0.00181259 | CF805725 | 571 | 136 | 151 | 90 | 1e−78 | response to hypoxia |
| 6032 | 2.862532435 | down | 0.00000441 | 0.000084 | CA802924 | 347 | 15 | 38 | 39 | 8e−04 | |
| 6033 | 2.861179511 | down | 0.00000326 | 0.0000692 | BM954488 | 282 | 19 | 30 | 63 | 2e−04 | |
| 6034 | 2.788025661 | down | 0.00000796 | 0.00012027 | BU761747 | 439 | 63 | 97 | 64 | 1e−32 | gibberellic acid biosynthesis |
| 6035 | 2.786910431 | down | 0.0000818 | 0.000597858 | CD404838 | 503 | 35 | 48 | 12 | 8e−13 | |
| 6036 | 2.786730428 | down | 0.00000102 | 0.0000339 | BE822236 | 582 | 49 | 171 | 28 | 2e−06 | |
| 6037 | 2.786142745 | down | 0.000331467 | 0.001628462 | BU547220 | 620 | 136 | 165 | 82 | 1e−75 | |
| 6038 | 2.784814866 | down | 0.00000207 | 0.0000521 | CD391382 | 623 | 67 | 90 | 74 | 2e−34 | WD-repeat cell cycle regulatory protein, |
| | | | | | | | | | | | G-protein coupled receptor protein |
| | | | | | | | | | | | signaling pathway |
| 6039 | 2.784353719 | down | 0.00000475 | 0.0000878 | BE331434 | 623 | 98 | 180 | 54 | 2e−47 | |
| 6040 | 2.784340717 | down | 0.00000414 | 0.0000804 | BG881526 | 510 | 131 | 136 | 96 | 5e−74 | |
| 6041 | 2.783774428 | down | 0.000000908 | 0.0000318 | BE021475 | 481 | 87 | 115 | 75 | 1e−50 | |
| 6042 | 2.783338591 | down | 0.0000811 | 0.000594322 | BG790426 | 478 | 39 | 130 | 30 | 7e−10 | |
| 6043 | 2.782751696 | down | 0.00000225 | 0.0000551 | BI497635 | 552 | 40 | 73 | 54 | 5e−16 | regulation of transcription |
| 6044 | 2.782433232 | down | 0.000120476 | 0.000787009 | BU964755 | 616 | 158 | 209 | 75 | 5e−90 | transport |
| 6045 | 2.782372744 | down | 0.00000558 | 0.000097 | BU549420 | 639 | 76 | 150 | 50 | 4e−32 | amino acid transport |
| 6046 | 2.781330045 | down | 0.0000768 | 0.000570201 | CF806668 | 720 | 65 | 140 | 46 | 2e−27 | |
| 6047 | 2.781243586 | down | 0.00000457 | 0.0000857 | AF002258.1 | | | | | | 4-coumarate:CoA ligase isoenzyme 3 |
| 6048 | 2.780536619 | down | 0.0000016 | 0.0000439 | BU765868 | 541 | 80 | 106 | 75 | 3e−47 | |
| 6049 | 2.780306323 | down | 0.000100962 | 0.000692234 | BU964503 | 444 | 81 | 123 | 65 | 3e−42 | |
| 6050 | 2.780296788 | down | 0.0000216 | 0.000236357 | AW310232 | 544 | 27 | 51 | 52 | 2e−07 | |
| 6051 | 2.77838424 | down | 0.0000176 | 0.000206127 | AW349967 | 641 | 53 | 74 | 71 | 8e−28 | |
| 6052 | 2.7777398 | down | 0.00000162 | 0.0000442 | CD394756 | 679 | 115 | 119 | 96 | 7e−64 | response to high light intensity |
| 6053 | 2.777359454 | down | 0.00000195 | 0.0000502 | BI426916 | 295 | 28 | 81 | 34 | 4e−06 | regulation of transcription\, DNA- |
| | | | | | | | | | | | dependent |
| 6054 | 2.776718092 | down | 0.00000682 | 0.000109405 | BM886923 | 545 | 38 | 72 | 52 | 3e−14 | |
| 6055 | 2.632970938 | down | 0.00000344 | 0.0000717 | BG406072 | 465 | 25 | 40 | 62 | 5e−10 | lipid catabolism |
| 6056 | 2.632842407 | down | 0.000301048 | 0.001522173 | BE823304 | 701 | 55 | 81 | 67 | 2e−27 | electron transport |
| 6057 | 2.632546661 | down | 0.000112239 | 0.000745992 | AY323131.1 | | | | | | GAD protein |
| 6058 | 2.6321339 | down | 0.0000135 | 0.000172988 | BM178355 | 561 | 110 | 163 | 67 | 4e−54 | morphogenesis |
| 6059 | 2.631082784 | down | 0.00012877 | 0.000825135 | BE805831 | 430 | 115 | 143 | 80 | 1e−65 | |
| 6060 | 2.631018579 | down | 0.00000762 | 0.000117192 | BU763098 | 301 | 20 | 69 | 28 | 4.2 | protein catabolism |
| 6061 | 2.630918316 | down | 0.00000234 | 0.0000565 | BI787214 | 571 | 33 | 93 | 35 | 3e−05 | hypothetical LOC547848, metal ion |
| | | | | | | | | | | | transport |
| 6062 | 2.630711282 | down | 0.00000172 | 0.0000461 | BM885192 | 465 | 11 | 36 | 30 | 1.0 | |
| 6063 | 2.630533759 | down | 0.00000629 | 0.000104925 | AW308885 | 428 | 32 | 44 | 72 | 7e−14 | |
| 6064 | 2.629698079 | down | 0.00090597 | 0.003400235 | CD407147 | 640 | 102 | 157 | 64 | 2e−53 | |
| 6065 | 2.629576943 | down | 0.00000176 | 0.0000466 | CD415327 | 692 | 53 | 97 | 54 | 2e−23 | |
| 6066 | 2.860359271 | down | 0.001611764 | 0.005139009 | BG511047 | | | | | | |
| 6067 | 2.860026238 | down | 0.00000689 | 0.000110029 | CD399954 | 465 | 26 | 49 | 53 | 2e−10 | biological_process unknown |
| 6068 | 2.859939377 | down | 0.000147902 | 0.000907681 | AW706614 | 440 | 16 | 49 | 32 | 7e−04 | |
| 6069 | 2.859805563 | down | 0.0000065 | 0.000106296 | BM094683 | | | | | | |
| 6070 | 2.858877721 | down | 0.00000303 | 0.0000664 | BG511742 | 357 | 15 | 59 | 25 | 0.51 | |
| 6071 | 2.858358998 | down | 0.000177919 | 0.001036112 | BQ612411 | 405 | 33 | 60 | 55 | 2e−13 | |
| 6072 | 2.858329194 | down | 0.000000997 | 0.0000336 | BI674271 | 474 | 12 | 28 | 42 | 0.79 | |
| 6073 | 2.85811665 | down | 0.0000769 | 0.000570812 | BU550979 | 626 | 81 | 107 | 75 | 1e−47 | |
| 6074 | 2.857765141 | down | 0.00000871 | 0.000127346 | CF806530 | 688 | 144 | 163 | 88 | 9e−86 | response to hypoxia |
| 6075 | 2.857408097 | down | 0.00000291 | 0.0000644 | BQ080608 | 569 | 22 | 86 | 25 | 0.30 | |
| 6076 | 2.857250524 | down | 0.00000416 | 0.0000807 | AW569278 | 336 | 8 | 15 | 53 | 9.4 | regulation of transcription\, DNA- |
| | | | | | | | | | | | dependent |
| 6077 | 2.856773466 | down | 0.0000739 | 0.000555836 | BM885321 | 570 | 80 | 151 | 52 | 1e−41 | electron transport |
| 6078 | 2.856325469 | down | 0.00000172 | 0.0000461 | CA801380 | 703 | 127 | 174 | 72 | 2e−65 | |
| 6079 | 2.85562445 | down | 0.00000863 | 0.000126508 | BE346991 | 571 | 97 | 113 | 85 | 2e−58 | |
| 6080 | 2.85536767 | down | 0.0000163 | 0.000196271 | BU926045 | 583 | 63 | 141 | 44 | 2e−21 | |
| 6081 | 2.855325176 | down | 0.0000387 | 0.000353797 | BE800712 | 601 | 78 | 168 | 46 | 2e−36 | |
| 6082 | 2.85473496 | down | 0.00002 | 0.000223716 | BF067605 | 543 | 60 | 177 | 33 | 2e−24 | metabolism |
| 6083 | 2.854399767 | down | 0.00000203 | 0.0000514 | CD405214 | 543 | 55 | 71 | 77 | 5e−22 | |
| 6084 | 2.85399903 | down | 0.00000122 | 0.0000371 | BU548301 | 582 | 39 | 55 | 70 | 3e−13 | regulation of transcription\, DNA- |
| | | | | | | | | | | | dependent |
| 6085 | 2.85342867 | down | 0.00000233 | 0.0000563 | CA802902 | 790 | 200 | 243 | 82 | e−110 | |
| 6086 | 2.850658377 | down | 0.0000127 | 0.00016513 | AW781786 | 398 | 66 | 135 | 48 | 2e−26 | protein amino acid phosphorylation |
| 6087 | 2.850334015 | down | 0.0000061 | 0.000102552 | BG044519 | 417 | 110 | 140 | 78 | 3e−60 | electron transport |
| 6088 | 2.848981847 | down | 0.0000237 | 0.000252713 | BG551078 | 472 | 44 | 131 | 33 | 1e−11 | |
| 6089 | 2.848809769 | down | 0.00000212 | 0.000053 | AW780839 | 313 | 21 | 68 | 30 | 2.4 | |
| 6090 | 2.848311029 | down | 0.00000115 | 0.0000361 | CF808975 | 550 | 171 | 183 | 83 | 2e−95 | acetyl-CoA biosynthesis |
| 6091 | 2.847590665 | down | 0.0000311 | 0.000306036 | CF806123 | 285 | 90 | 92 | 97 | 1e−49 | |
| 6092 | 2.84755955 | down | 0.00000773 | 0.000118008 | BU964854 | 583 | 96 | 159 | 60 | 4e−47 | |
| 6093 | 2.846786054 | down | 0.000215373 | 0.00119158 | CD401722 | 702 | 47 | 71 | 66 | 3e−24 | |
| 6094 | 2.846578766 | down | 0.00000261 | 0.0000604 | BG362703 | 541 | 30 | 37 | 81 | 2e−11 | |
| 6095 | 2.845767602 | down | 0.00000133 | 0.0000391 | CD396138 | 714 | 73 | 94 | 77 | 4e−36 | |
| 6096 | 2.844082857 | down | 0.000162696 | 0.000973226 | CD415914 | 589 | 58 | 62 | 93 | 1e−29 | |
| 6097 | 2.842103705 | down | 0.00018408 | 0.001060819 | BG352380 | 589 | 75 | 197 | 38 | 2e−35 | electron transport |
| 6098 | 2.842014085 | down | 0.0000108 | 0.000148221 | CF808054 | 444 | 104 | 148 | 70 | 2e−60 | L-serine biosynthesis |
| 6099 | 2.841712038 | down | 0.00000767 | 0.000117604 | BE658250 | 793 | 83 | 105 | 79 | 1e−52 | carbohydrate biosynthesis |
| 6100 | 2.840460481 | down | 0.000203325 | 0.001141304 | AB004271.1 | | | | | | |
| 6101 | 2.84013003 | down | 0.0000581 | 0.000470008 | AW349908 | 619 | 86 | 153 | 56 | 7e−34 | |
| 6102 | 2.83954097 | down | 0.000000509 | 0.0000225 | BU546224 | 454 | 32 | 86 | 37 | 4e−13 | electron transport |
| 6103 | 2.839364637 | down | 0.0000967 | 0.000674098 | BF009291 | 434 | 58 | 74 | 78 | 2e−29 | phosphate transport |
| 6104 | 2.839310672 | down | 0.00000362 | 0.0000739 | AW310947 | 524 | 19 | 32 | 59 | 7e−05 | |
| 6105 | 2.838814468 | down | 0.00000652 | 0.000106503 | BE473599 | 530 | 145 | 179 | 81 | 7e−82 | transport |
| 6106 | 2.838588409 | down | 0.000012 | 0.000158814 | CF806702 | 652 | 59 | 197 | 29 | 5e−16 | |
| 6107 | 2.83645147 | down | 0.000105658 | 0.000714403 | AW348373 | 461 | 32 | 53 | 60 | 5e−10 | electron transport |
| 6108 | 2.834960131 | down | 0.000489558 | 0.002164962 | AI461202 | 468 | 87 | 154 | 56 | 9e−42 | |
| 6109 | 2.834906364 | down | 0.0000203 | 0.000226043 | BE610184 | 547 | 105 | 181 | 58 | 1e−56 | electron transport |
| 6110 | 2.834669546 | down | 0.00000109 | 0.000035 | CF806914 | 709 | 78 | 248 | 31 | 2e−30 | metabolism |
| 6111 | 2.834507773 | down | 0.000692947 | 0.002783698 | BF068658 | 432 | 47 | 161 | 29 | 2e−04 | regulation of transcription |
| 6112 | 2.834358857 | down | 0.001050055 | 0.003775982 | AW185298 | 427 | 73 | 92 | 79 | 5e−40 | |
| 6113 | 2.834106807 | down | 0.0000853 | 0.000615324 | AW570288 | 339 | 27 | 43 | 62 | 2e−07 | |
| 6114 | 2.833999337 | down | 0.0000178 | 0.000207266 | U87907.1 | | | | | | alternative oxidase 2b |
| 6115 | 2.833578486 | down | 0.00000708 | 0.000112018 | CA801220 | 767 | 86 | 190 | 45 | 3e−31 | |
| 6116 | 2.832539453 | down | 0.00000179 | 0.0000473 | AW102068 | 470 | 53 | 82 | 64 | 6e−27 | lipid catabolism |
| 6117 | 2.832372038 | down | 0.000101402 | 0.000693984 | BQ611525 | 512 | 87 | 167 | 52 | 1e−41 | |
| 6118 | 2.832343484 | down | 0.0000067 | 0.000108286 | BU548404 | 618 | 71 | 118 | 60 | 2e−38 | |
| 6119 | 2.831773603 | down | 0.000019 | 0.000216456 | CD395659 | 636 | 62 | 81 | 76 | 2e−29 | |
| 6120 | 2.831318858 | down | 0.00000144 | 0.000041 | BU765143 | 589 | 82 | 98 | 83 | 3e−44 | |
| 6121 | 2.830913209 | down | 0.000000679 | 0.0000269 | BU547481 | 598 | 84 | 170 | 49 | 1e−46 | |
| 6122 | 2.830337605 | down | 0.000484707 | 0.002149725 | BQ252738 | 419 | 62 | 101 | 61 | 3e−28 | N-terminal protein myristoylation |
| 6123 | 2.829729992 | down | 0.00000818 | 0.00012225 | BU577692 | 582 | 119 | 194 | 61 | 2e−70 | protein catabolism |
| 6124 | 2.828939472 | down | 0.000570085 | 0.002426247 | CD394418 | 574 | 51 | 64 | 79 | 4e−27 | |
| 6125 | 2.828932046 | down | 0.00000988 | 0.000139117 | AI988141 | 675 | 36 | 83 | 43 | 5e−13 | apoptosis |
| 6126 | 2.827272737 | down | 0.000012 | 0.000158814 | CA801989 | 662 | 167 | 219 | 76 | 8e−92 | defense response |
| 6127 | 2.826929887 | down | 0.0000139 | 0.000175751 | BE473523 | 464 | 27 | 32 | 84 | 8e−11 | carbohydrate metabolism |
| 6128 | 2.826928136 | down | 0.000397337 | 0.001859812 | CK606407 | 469 | 62 | 75 | 82 | 2e−29 | |
| 6129 | 2.826668807 | down | 0.00000312 | 0.0000676 | BU546941 | 643 | 39 | 99 | 39 | 1e−12 | aging |
| 6130 | 2.826561505 | down | 0.00000691 | 0.000110351 | BG237790 | | | | | | |
| 6131 | 2.826316105 | down | 0.0000525 | 0.000437727 | BU925851 | 629 | 95 | 148 | 64 | 5e−50 | protein amino acid phosphorylation |
| 6132 | 2.825890346 | down | 0.00000534 | 0.0000946 | BI315570 | 497 | 136 | 165 | 82 | 3e−77 | protein amino acid phosphorylation |
| 6133 | 2.82534798 | down | 0.0000316 | 0.000308768 | AI973546 | 435 | 13 | 42 | 30 | 1.1 | |
| 6134 | 2.82432061 | down | 0.00000159 | 0.0000438 | CF808605 | 708 | 157 | 219 | 71 | 3e−90 | |
| 6135 | 2.824286258 | down | 0.00000742 | 0.000115521 | CF806328 | 713 | 22 | 77 | 28 | 1.7 | |
| 6136 | 2.82410515 | down | 0.000014 | 0.000176849 | BI967760 | 779 | 27 | 32 | 84 | 2e−09 | |
| 6137 | 2.823376067 | down | 0.000250851 | 0.001331197 | AW756161 | 548 | 75 | 137 | 54 | 2e−33 | |
| 6138 | 2.823201166 | down | 0.00000687 | 0.000109898 | BQ610593 | 299 | 20 | 60 | 33 | 0.13 | |
| 6139 | 2.823055279 | down | 0.001206901 | 0.004167863 | BG363199 | 539 | 88 | 167 | 52 | 2e−41 | protein amino acid phosphorylation |
| 6140 | 2.822821715 | down | 0.00000242 | 0.0000576 | D50870.1 | | | | | | mitotic cyclin a2-type |
| 6141 | 2.822408346 | down | 0.00000573 | 0.0000986 | BF598665 | 467 | 37 | 71 | 52 | 1e−13 | metal ion transport |
| 6142 | 2.822355621 | down | 0.000000676 | 0.0000269 | BE820331 | 573 | 31 | 54 | 57 | 8e−13 | |
| 6143 | 2.822096945 | down | 0.00000387 | 0.0000772 | BM307460 | 489 | 12 | 24 | 50 | 0.10 | |
| 6144 | 2.822038627 | down | 0.000000478 | 0.0000217 | CA803053 | 311 | 10 | 31 | 32 | 7.1 | oligopeptide transport |
| 6145 | 2.82136626 | down | 0.000256931 | 0.001355236 | BQ299017 | 362 | 12 | 34 | 35 | 0.92 | proteolysis and peptidolysis |
| 6146 | 2.821279059 | down | 0.00000152 | 0.0000424 | BI970555 | 757 | 109 | 138 | 78 | 2e−57 | protein amino acid phosphorylation |
| 6147 | 2.820970471 | down | 0.000793805 | 0.003076072 | BG511051 | 441 | 13 | 34 | 38 | 2.0 | |
| 6148 | 2.81986014 | down | 0.000093 | 0.000654211 | BE822362 | 489 | 85 | 112 | 75 | 9e−40 | |
| 6149 | 2.819646103 | down | 0.00000802 | 0.000120862 | BM085755 | 490 | 113 | 163 | 69 | 3e−61 | |
| 6150 | 2.818688769 | down | 0.00000391 | 0.0000776 | CD413710 | 362 | 35 | 42 | 83 | 2e−14 | protein biosynthesis |
| 6151 | 2.817811863 | down | 0.00000299 | 0.0000658 | BI969522 | 734 | 40 | 50 | 80 | 2e−16 | |
| 6152 | 2.817554068 | down | 0.000018 | 0.000208414 | BQ611085 | 541 | 133 | 177 | 75 | 3e−66 | |
| 6153 | 2.817046614 | down | 0.000122256 | 0.000795457 | CD404838 | 503 | 35 | 48 | 72 | 8e−13 | |
| 6154 | 2.816991882 | down | 0.00000956 | 0.000135745 | BG406101 | 370 | 57 | 97 | 58 | 5e−28 | |
| 6155 | 2.816793171 | down | 0.000000943 | 0.0000325 | BG509076 | 311 | 16 | 33 | 48 | 0.026 | steroid metabolism |
| 6156 | 2.816006706 | down | 0.00000341 | 0.0000713 | BI699782 | 574 | 71 | 184 | 38 | 9e−30 | metabolism |
| 6157 | 2.815969932 | down | 0.0000174 | 0.000204458 | BE609266 | 446 | 51 | 147 | 34 | 6e−15 | |
| 6158 | 2.815926832 | down | 0.00000645 | 0.000105861 | BI674278 | 471 | 22 | 30 | 73 | 9e−08 | protein amino acid phosphorylation |
| 6159 | 2.815352548 | down | 0.00000157 | 0.0000435 | CD414878 | 606 | 85 | 86 | 98 | 1e−41 | nucleosome assembly |
| 6160 | 2.814982178 | down | 0.00000645 | 0.000105827 | BU763592 | 419 | 15 | 67 | 22 | 6.6 | regulation of transcription |
| 6161 | 2.814428664 | down | 0.0000631 | 0.000497577 | BE058832 | 471 | 12 | 27 | 44 | 8.7 | |
| 6162 | 2.814351173 | down | 0.00000282 | 0.000063 | CF809129 | 694 | 65 | 132 | 49 | 1e−27 | |
| 6163 | 2.814308312 | down | 0.000102009 | 0.000697268 | AW307334 | 237 | 14 | 39 | 35 | 5.4 | |
| 6164 | 2.813610037 | down | 0.000255067 | 0.001347482 | BM092012 | 239 | 14 | 43 | 32 | 5.4 | protein amino acid phosphorylation |
| 6165 | 2.813486687 | down | 0.00000394 | 0.000078 | BI426447 | 376 | 69 | 77 | 89 | 2e−41 | carbohydrate biosynthesis |
| 6166 | 2.812987587 | down | 0.00000482 | 0.0000886 | BU550686 | | | | | | |
| 6167 | 2.812378282 | down | 0.001065237 | 0.003817436 | BE822965 | 713 | 45 | 117 | 38 | 2e−10 | regulation of transcription\, DNA- |
| | | | | | | | | | | | dependent |
| 6168 | 2.812008737 | down | 0.0000491 | 0.00041764 | BI971058 | 775 | 133 | 208 | 63 | 3e−74 | proteolysis and peptidolysis |
| 6169 | 2.811987748 | down | 0.00000183 | 0.000048 | BE657998 | 692 | 57 | 84 | 67 | 7e−25 | |
| 6170 | 2.811891061 | down | 0.0000112 | 0.000151484 | AW757139 | 414 | 23 | 39 | 58 | 0.041 | regulation of transcription\, DNA- |
| | | | | | | | | | | | dependent |
| 6171 | 2.811730986 | down | 0.00000547 | 0.0000959 | AB007503.1 | | | | | | squalene synthase |
| 6172 | 2.811552354 | down | 0.00000239 | 0.0000572 | BG508441 | 430 | 68 | 124 | 54 | 3e−37 | proteolysis and peptidolysis |
| 6173 | 2.810849533 | down | 0.00000644 | 0.000105803 | AF030231.1 | | | | | | sucrose synthase |
| 6174 | 2.810575671 | down | 0.0000271 | 0.000277568 | BE658807 | 382 | 14 | 38 | 36 | 1.1 | |
| 6175 | 2.810333424 | down | 0.00000659 | 0.000107091 | BI968067 | 791 | 140 | 206 | 67 | 2e−84 | cell wall modification |
| 6176 | 2.809950602 | down | 0.0000225 | 0.000243882 | BE800855 | 517 | 68 | 162 | 41 | 5e−26 | regulation of transcription\, DNA- |
| | | | | | | | | | | | dependent |
| 6177 | 2.808247312 | down | 0.0000746 | 0.000559321 | AW350303 | 658 | 28 | 104 | 26 | 0.035 | |
| 6178 | 2.807600848 | down | 0.000142639 | 0.000886665 | AW596381 | 543 | 58 | 78 | 74 | 6e−30 | |
| 6179 | 2.807569023 | down | 0.000142545 | 0.000886228 | AW234624 | 596 | 150 | 192 | 78 | 2e−84 | electron transport |
| 6180 | 2.806394502 | down | 0.0000093 | 0.000132901 | BI317137 | 469 | 108 | 155 | 69 | 1e−56 | |
| 6181 | 2.805330447 | down | 0.00000609 | 0.000102493 | CA820659 | 562 | 19 | 70 | 27 | 0.020 | |
| 6182 | 2.804973517 | down | 0.00000549 | 0.0000961 | CA819603 | 598 | 74 | 186 | 39 | 1e−34 | |
| 6183 | 2.804592807 | down | 0.00000476 | 0.0000878 | BU762792 | 404 | 36 | 59 | 61 | 2e−14 | protein folding |
| 6184 | 2.804380219 | down | 0.00000178 | 0.0000471 | AW309700 | 668 | 129 | 158 | 81 | 1e−68 | terpenoid biosynthesis |
| 6185 | 2.803868695 | down | 0.0000884 | 0.00063142 | BQ611705 | 270 | 16 | 46 | 34 | 0.84 | |
| 6186 | 2.8035084 | down | 0.00000269 | 0.0000614 | AW277772 | 608 | 141 | 202 | 69 | 5e−82 | photomorphogenesis |
| 6187 | 2.801546655 | down | 0.0000122 | 0.000161084 | AW755562 | 554 | 145 | 168 | 86 | 3e−81 | pyridoxine biosynthesis |
| 6188 | 2.799595348 | down | 0.00000456 | 0.0000855 | BQ612000 | 271 | 12 | 36 | 33 | 1.4 | DNA recombination |
| 6189 | 2.799456418 | down | 0.000037 | 0.00034356 | CF807411 | 678 | 105 | 188 | 55 | 2e−49 | transport |
| 6190 | 2.799341389 | down | 0.001081699 | 0.003860616 | BQ612376 | 500 | 13 | 43 | 30 | 0.90 | |
| 6191 | 2.798828555 | down | 0.00064998 | 0.002662931 | BE059130 | | | | | | |
| 6192 | 2.798603045 | down | 0.000176215 | 0.001030533 | AW350818 | 721 | 13 | 43 | 30 | 2.2 | electron transport |
| 6193 | 2.798443557 | down | 0.0000075 | 0.000116095 | CD415285 | 325 | 12 | 28 | 42 | 1.4 | |
| 6194 | 2.798068789 | down | 0.00000263 | 0.0000607 | BE475213 | 425 | 31 | 139 | 22 | 5e−06 | |
| 6195 | 2.797710016 | down | 0.0000182 | 0.000210468 | CD397366 | 650 | 106 | 166 | 63 | 2e−55 | |
| 6196 | 2.797442266 | down | 0.00000564 | 0.0000977 | CF809244 | 533 | 19 | 56 | 33 | 0.054 | |
| 6197 | 2.797135083 | down | 0.0000634 | 0.000499469 | BI785751 | 558 | 155 | 170 | 91 | 3e−86 | (G. max) mRNA for proliferating cell |
| | | | | | | | | | | | nuclear antigen (PCNA), partial, |
| | | | | | | | | | | | regulation of DNA replication |
| 6198 | 2.797033358 | down | 0.000014 | 0.000176962 | BI972112 | 419 | 47 | 76 | 61 | 3e−23 | protein amino acid phosphorylation |
| 6199 | 2.796868199 | down | 0.00000371 | 0.0000752 | BU545109 | 567 | 14 | 19 | 73 | 0.10 | |
| 6200 | 2.796244064 | down | 0.000102398 | 0.000699149 | BM522616 | | | | | | |
| 6201 | 2.794795134 | down | 0.000484496 | 0.002149725 | BF067528 | 467 | 107 | 164 | 65 | 2e−51 | |
| 6202 | 2.794144172 | down | 0.0000138 | 0.000175004 | AI974100 | 273 | 14 | 32 | 43 | 0.84 | protein amino acid phosphorylation |
| 6203 | 2.793784961 | down | 0.0000564 | 0.000461288 | BI497616 | 549 | 129 | 181 | 71 | 2e−74 | protein amino acid phosphorylation |
| 6204 | 2.793487589 | down | 0.0000817 | 0.00059683 | BG359742 | 362 | 20 | 42 | 47 | 1.2 | |
| 6205 | 2.793204387 | down | 0.00000804 | 0.00012101 | BE822228 | 603 | 55 | 106 | 51 | 3e−25 | |
| 6206 | 2.792446183 | down | 0.00019758 | 0.001117223 | AI495316 | 644 | 59 | 144 | 40 | 8e−23 | regulation of transcription\, DNA- |
| | | | | | | | | | | | dependent |
| 6207 | 2.792154353 | down | 0.000000815 | 0.0000298 | BE346971 | 584 | 43 | 97 | 44 | 2e−12 | |
| 6208 | 2.791911646 | down | 0.0000326 | 0.000315668 | CA937162 | 424 | 12 | 31 | 38 | 0.47 | |
| 6209 | 2.791650039 | down | 0.00000104 | 0.0000342 | BU550633 | 694 | 97 | 127 | 76 | 2e−48 | regulation of gene expression\, |
| | | | | | | | | | | | epigenetic |
| 6210 | 2.790985067 | down | 0.0000116 | 0.000154676 | CD404506 | 632 | 67 | 110 | 60 | 3e−26 | |
| 6211 | 2.789119218 | down | 0.00000964 | 0.000136581 | CF806677 | 696 | 97 | 173 | 56 | 7e−52 | |
| 6212 | 2.788692271 | down | 0.0000134 | 0.00017107 | BM308031 | 614 | 115 | 197 | 58 | 3e−58 | |
| 6213 | 2.788623809 | down | 0.000509161 | 0.002226444 | AW350475 | 780 | 120 | 143 | 83 | 1e−67 | water transport |
| 6214 | 2.788610039 | down | 0.00000284 | 0.0000633 | BI968670 | 570 | 68 | 109 | 62 | 6e−37 | |
| 6215 | 2.788129738 | down | 0.00000553 | 0.0000965 | CD415130 | 674 | 82 | 158 | 51 | 2e−37 | regulation of transcription |
| 6216 | 2.788102309 | down | 0.0000713 | 0.000542385 | BQ611691 | 415 | 12 | 22 | 54 | 2.2 | |
| 6217 | 2.776323991 | down | 0.00000461 | 0.000086 | BE191451 | 607 | 120 | 203 | 59 | 3e−58 | |
| 6218 | 2.775467717 | down | 0.00000444 | 0.0000843 | BU577083 | 573 | 82 | 135 | 60 | 2e−43 | |
| 6219 | 2.77415461 | down | 0.00000116 | 0.0000363 | BF067657 | 427 | 87 | 119 | 73 | 1e−45 | ciliary/flagellar motility |
| 6220 | 2.774125553 | down | 0.000638551 | 0.002630142 | BG238407 | 701 | 147 | 164 | 89 | 2e−81 | metabolism |
| 6221 | 2.77323321 | down | 0.0000306 | 0.000302372 | BI320922 | 460 | 50 | 82 | 60 | 2e−24 | basic amino acid transport |
| 6222 | 2.773217007 | down | 0.00000151 | 0.0000423 | AY247754.1 | | | | | | hypothetical LOC547825 |
| 6223 | 2.772474205 | down | 0.00000487 | 0.0000892 | BE822342 | 540 | 41 | 101 | 40 | 3e−12 | |
| 6224 | 2.772227636 | down | 0.00000217 | 0.0000536 | BG363209 | 478 | 60 | 105 | 57 | 1e−30 | |
| 6225 | 2.772190832 | down | 0.00000926 | 0.000132671 | BU548684 | 687 | 94 | 136 | 69 | 3e−50 | SferH-2 mRNA for ferritin, iron ion |
| | | | | | | | | | | | transport |
| 6226 | 2.771764649 | down | 0.00000122 | 0.0000372 | AW397627 | 358 | 50 | 66 | 75 | 2e−27 | proteolysis and peptidolysis |
| 6227 | 2.770685705 | down | 0.000313137 | 0.001566061 | BE659858 | 374 | 11 | 23 | 47 | 6.5 | |
| 6228 | 2.770554226 | down | 0.0000968 | 0.000674333 | AW832496 | 306 | 45 | 70 | 64 | 9e−19 | regulation of transcription |
| 6229 | 2.770411348 | down | 0.00000453 | 0.0000852 | CD487688 | 630 | 56 | 91 | 61 | 1e−26 | protein amino acid phosphorylation |
| 6230 | 2.769879789 | down | 0.000538719 | 0.002322982 | AW307237 | 314 | 17 | 33 | 51 | 5e−04 | glucosinolate biosynthesis |
| 6231 | 2.769051678 | down | 0.0000366 | 0.000341488 | BU547416 | 663 | 89 | 152 | 58 | 6e−44 | |
| 6232 | 2.769041817 | down | 0.0000184 | 0.00021144 | AI736226 | 180 | 14 | 34 | 41 | 0.004 | |
| 6233 | 2.768476126 | down | 0.000007 | 0.000111171 | AW309358 | 657 | 28 | 105 | 26 | 0.004 | |
| 6234 | 2.768056459 | down | 0.0000032 | 0.0000684 | CF807799 | 706 | 134 | 184 | 72 | 6e−70 | glycolysis |
| 6235 | 2.768033212 | down | 0.000023 | 0.000247534 | CD407259 | 675 | 84 | 126 | 66 | 1e−35 | |
| 6236 | 2.767902325 | down | 0.0000158 | 0.000192003 | BU761202 | 617 | 122 | 192 | 63 | 3e−68 | |
| 6237 | 2.767595312 | down | 0.0000314 | 0.000307687 | BI788044 | 365 | 17 | 39 | 43 | 0.55 | aromatic amino acid family metabolism |
| 6238 | 2.766915982 | down | 0.00000587 | 0.000100049 | BG789735 | 540 | 55 | 178 | 30 | 2e−20 | antibiotic biosynthesis |
| 6239 | 2.765501331 | down | 0.00000379 | 0.0000762 | CK606168 | 822 | 149 | 258 | 57 | 4e−77 | biological_process unknown |
| 6240 | 2.765436237 | down | 0.0000167 | 0.000198784 | BG511034 | 546 | 141 | 182 | 77 | 2e−80 | protein amino acid phosphorylation |
| 6241 | 2.765317313 | down | 0.00000429 | 0.0000825 | AW350699 | 663 | 17 | 49 | 34 | 1.9 | nucleotide-excision repair |
| 6242 | 2.764931457 | down | 0.0000121 | 0.000159302 | BU550791 | 681 | 48 | 60 | 80 | 4e−30 | |
| 6243 | 2.76469833 | down | 0.00000464 | 0.0000864 | CD399954 | 465 | 26 | 49 | 53 | 2e−10 | biological_process unknown |
| 6244 | 2.764692161 | down | 0.0000206 | 0.00022876 | CA801365 | 385 | 29 | 40 | 72 | 1e−08 | regulation of transcription\, DNA- |
| | | | | | | | | | | | dependent |
| 6245 | 2.764406258 | down | 0.000343626 | 0.001669699 | BI973984 | 576 | 113 | 191 | 59 | 1e−55 | DNA replication |
| 6246 | 2.764277448 | down | 0.000355815 | 0.001714639 | BU091407 | 406 | 51 | 141 | 36 | 1e−21 | |
| 6247 | 2.763932992 | down | 0.000155772 | 0.000942663 | CD404769 | 467 | 27 | 35 | 77 | 6e−09 | metabolism |
| 6248 | 2.763540336 | down | 0.0000344 | 0.000327564 | BU547997 | 591 | 38 | 72 | 52 | 2e−12 | |
| 6249 | 2.762916989 | down | 0.0000037 | 0.0000751 | AI856394 | 457 | 22 | 73 | 30 | 0.73 | |
| 6250 | 2.762110227 | down | 0.001765432 | 0.00548303 | BI469429 | 566 | 55 | 121 | 45 | 2e−26 | electron transport |
| 6251 | 2.761710989 | down | 0.00000601 | 0.000101579 | CA802033 | 301 | 18 | 45 | 40 | 1.5 | |
| 6252 | 2.761291528 | down | 0.00000653 | 0.000106503 | BI969830 | 800 | 62 | 145 | 42 | 7e−28 | |
| 6253 | 2.761035774 | down | 0.0000847 | 0.000612132 | AW203618 | 424 | 14 | 28 | 50 | 1.4 | |
| 6254 | 2.760753718 | down | 0.0000586 | 0.00047303 | AW311648 | | | | | | |
| 6255 | 2.760436459 | down | 0.00018798 | 0.001077412 | BM178538 | 393 | 16 | 46 | 34 | 0.14 | regulation of translational initiation |
| 6256 | 2.759253915 | down | 0.00000743 | 0.000115625 | BI892641 | 570 | 92 | 135 | 68 | 2e−55 | dicarboxylic acid transport |
| 6257 | 2.759134334 | down | 0.000048 | 0.000411874 | BE609741 | 266 | 33 | 71 | 46 | 2e−10 | protein amino acid phosphorylation |
| 6258 | 2.757674348 | down | 0.0000576 | 0.000467579 | CD408191 | 681 | 114 | 121 | 94 | 3e−61 | glycolysis |
| 6259 | 2.756950176 | down | 0.0000242 | 0.000256666 | BM524482 | 576 | 148 | 191 | 77 | 3e−83 | steroid biosynthesis |
| 6260 | 2.756905909 | down | 0.00000675 | 0.000108736 | BQ273679 | 579 | 67 | 114 | 58 | 6e−39 | |
| 6261 | 2.756780229 | down | 0.00000275 | 0.000062 | CD407905 | 607 | 70 | 87 | 80 | 8e−37 | protein biosynthesis |
| 6262 | 2.756727437 | down | 0.0000342 | 0.000326244 | BM307497 | 402 | 48 | 59 | 81 | 2e−23 | |
| 6263 | 2.756232134 | down | 0.000172772 | 0.001017483 | BF596816 | 608 | 158 | 203 | 77 | 4e−87 | protein amino acid phosphorylation |
| 6264 | 2.755471619 | down | 0.0000235 | 0.00025161 | CA853885 | 524 | 107 | 165 | 64 | 1e−55 | proline biosynthesis |
| 6265 | 2.755425664 | down | 0.00000117 | 0.0000363 | BI471633 | 504 | 94 | 117 | 80 | 1e−49 | |
| 6266 | 2.74278016 | down | 0.0000556 | 0.00045587 | CD398479 | 710 | 22 | 75 | 29 | 0.57 | protein amino acid phosphorylation |
| 6267 | 2.742551064 | down | 0.0000217 | 0.000237277 | CD398858 | 498 | 21 | 39 | 53 | 3e−08 | hypothetical LOC547938, pathogenesis |
| 6268 | 2.742240689 | down | 0.000021 | 0.000231638 | CF805768 | 552 | 99 | 115 | 86 | 3e−56 | electron transport |
| 6269 | 2.742144891 | down | 0.000058 | 0.000469661 | BI971515 | 795 | 72 | 160 | 45 | 3e−32 | regulation of transcription\, DNA- |
| | | | | | | | | | | | dependent |
| 6270 | 2.741430932 | down | 0.00000174 | 0.0000462 | CF806960 | 612 | 158 | 185 | 85 | 1e−88 | |
| 6271 | 2.740152772 | down | 0.0000134 | 0.000171238 | BQ629078 | 431 | 21 | 69 | 30 | 0.29 | |
| 6272 | 2.739807966 | down | 0.00000221 | 0.0000544 | BU765373 | 575 | 32 | 144 | 22 | 0.001 | |
| 6273 | 2.738185276 | down | 0.000000908 | 0.0000318 | CA799417 | 687 | 53 | 97 | 54 | 2e−24 | |
| 6274 | 2.73787514 | down | 0.00000981 | 0.000138334 | BU762927 | 458 | 50 | 52 | 96 | 1e−23 | microtubule cytoskeleton organization |
| | | | | | | | | | | | and biogenesis |
| 6275 | 2.73739993 | down | 0.000129771 | 0.000829996 | CA937008 | 560 | 63 | 110 | 57 | 2e−26 | |
| 6276 | 2.735801922 | down | 0.00000445 | 0.0000843 | CF806858 | 706 | 163 | 211 | 77 | 9e−95 | |
| 6277 | 2.735612594 | down | 0.00000354 | 0.0000729 | AW234095 | 442 | 50 | 131 | 38 | 7e−11 | |
| 6278 | 2.732762873 | down | 0.000116596 | 0.000767423 | AW757083 | 481 | 48 | 60 | 80 | 6e−23 | transport |
| 6279 | 2.732445725 | down | 0.00000963 | 0.000136555 | BQ611985 | 186 | 13 | 27 | 48 | 1.4 | |
| 6280 | 2.731599076 | down | 0.0000769 | 0.000571125 | BU577013 | 483 | 52 | 71 | 73 | 4e−24 | protein amino acid dephosphorylation |
| 6281 | 2.731234674 | down | 0.00000348 | 0.0000722 | BG550828 | 390 | 9 | 26 | 34 | 9.6 | |
| 6282 | 2.731091138 | down | 0.00000452 | 0.0000851 | CD405048 | 381 | 14 | 49 | 28 | 0.21 | |
| 6283 | 2.730576718 | down | 0.00000432 | 0.0000828 | BG507427 | 509 | 16 | 53 | 30 | 1.2 | |
| 6284 | 2.7304645 | down | 0.0000255 | 0.000266283 | BM188470 | 550 | 131 | 183 | 71 | 1e−77 | |
| 6285 | 2.729583777 | down | 0.0000113 | 0.000153042 | AW100596 | 309 | 36 | 58 | 62 | 3e−15 | transition metal ion homeostasis |
| 6286 | 2.728477377 | down | 0.00000733 | 0.000114471 | BI972431 | 581 | 101 | 186 | 54 | 1e−48 | response to toxin |
| 6287 | 2.72764414 | down | 0.000225458 | 0.001230454 | AI974052 | 450 | 10 | 17 | 58 | 0.19 | |
| 6288 | 2.727187277 | down | 0.000190736 | 0.001088838 | BF069863 | 582 | 168 | 196 | 85 | 4e−98 | cellulose biosynthesis |
| 6289 | 2.726010983 | down | 0.00000229 | 0.0000555 | BI945276 | 286 | 14 | 41 | 34 | 0.37 | |
| 6290 | 2.7258218 | down | 0.0000436 | 0.000386227 | AW759220 | 494 | 96 | 164 | 58 | 2e−50 | transport |
| 6291 | 2.72570788 | down | 0.0000193 | 0.000219577 | BE822305 | 644 | 82 | 121 | 67 | 3e−47 | |
| 6292 | 2.725382876 | down | 0.0000302 | 0.000299328 | CD392171 | 633 | 28 | 42 | 66 | 5e−09 | |
| 6293 | 2.725126518 | down | 0.00000073 | 0.0000281 | AW349693 | 668 | 91 | 139 | 65 | 9e−51 | transport |
| 6294 | 2.725073288 | down | 0.0000239 | 0.000254646 | BI968723 | 753 | 98 | 175 | 56 | 2e−55 | |
| 6295 | 2.72401019 | down | 0.00000398 | 0.0000784 | AW311395 | 347 | 17 | 48 | 35 | 0.29 | |
| 6296 | 2.723818794 | down | 0.0000276 | 0.000281287 | AW394771 | 449 | 83 | 103 | 80 | 1e−46 | electron transport |
| 6297 | 2.723349137 | down | 0.000003 | 0.0000659 | AW780839 | 313 | 21 | 68 | 30 | 2.4 | |
| 6298 | 2.72314322 | down | 0.000226357 | 0.001233677 | AW508335 | 451 | 96 | 152 | 63 | 6e−50 | protein amino acid phosphorylation |
| 6299 | 2.723011318 | down | 0.0000329 | 0.0003173 | CD413667 | 322 | 11 | 23 | 47 | 5.4 | |
| 6300 | 2.722773554 | down | 0.0000054 | 0.0000952 | CF807468 | 567 | 105 | 151 | 69 | 2e−56 | |
| 6301 | 2.722661587 | down | 0.00000718 | 0.000113104 | CA782921 | 757 | 137 | 245 | 55 | 2e−75 | response to sucrose stimulus |
| 6302 | 2.722113812 | down | 0.00000117 | 0.0000363 | AI441316 | 291 | 18 | 69 | 26 | 0.015 | |
| 6303 | 2.72138781 | down | 0.000248603 | 0.001322437 | CA938643 | 603 | 124 | 188 | 65 | 1e−74 | |
| 6304 | 2.720997288 | down | 0.0000176 | 0.000205986 | CA799713 | 745 | 159 | 174 | 91 | 3e−95 | gluconeogenesis |
| 6305 | 2.720699438 | down | 0.0000613 | 0.000487243 | BG508510 | 334 | 14 | 38 | 36 | 0.85 | |
| 6306 | 2.720544237 | down | 0.0000154 | 0.000189144 | AW595946 | 509 | 131 | 161 | 81 | 4e−74 | |
| 6307 | 2.720156135 | down | 0.0000141 | 0.000177492 | AW350021 | 751 | 143 | 175 | 81 | 5e−82 | |
| 6308 | 2.720089424 | down | 0.0000338 | 0.000323636 | CA935466 | 312 | 9 | 28 | 32 | 7.1 | ion transport |
| 6309 | 2.719818113 | down | 0.0000228 | 0.000246298 | BE821099 | 662 | 66 | 83 | 79 | 5e−33 | transport |
| 6310 | 2.719800303 | down | 0.00000101 | 0.0000337 | BI974043 | 569 | 45 | 83 | 54 | 7e−22 | potassium ion transport |
| 6311 | 2.719031433 | down | 0.0000182 | 0.000209952 | AI440673 | 434 | 58 | 130 | 44 | 2e−22 | |
| 6312 | 2.718949272 | down | 0.0000279 | 0.000282752 | AW704352 | 547 | 81 | 144 | 56 | 4e−39 | |
| 6313 | 2.718869943 | down | 0.0000186 | 0.000212882 | BU084219 | 556 | 64 | 79 | 81 | 1e−33 | auxin mediated signaling |
| 6314 | 2.718256916 | down | 0.0000055 | 0.0000962 | CD401978 | 674 | 147 | 156 | 94 | 1e−82 | |
| 6315 | 2.71825643 | down | 0.0000177 | 0.000207083 | CA803112 | 412 | 15 | 63 | 23 | 2.2 | |
| 6316 | 2.718192595 | down | 0.00000143 | 0.0000408 | CD413696 | 298 | 9 | 35 | 25 | 1.9 | nucleotide-excision repair |
| 6317 | 2.718083452 | down | 0.0000197 | 0.000221672 | AI930826 | 561 | 85 | 169 | 50 | 1e−31 | transcription |
| 6318 | 2.717945813 | down | 0.000000697 | 0.0000274 | BI967831 | 688 | 134 | 151 | 88 | 8e−72 | |
| 6319 | 2.71730119 | down | 0.00000761 | 0.000117185 | AW781103 | 435 | 41 | 79 | 51 | 5e−22 | proteolysis and peptidolysis |
| 6320 | 2.717099355 | down | 0.0000299 | 0.000297021 | CD397017 | 553 | 21 | 79 | 26 | 4.1 | |
| 6321 | 2.716957478 | down | 0.0000171 | 0.000201703 | CD407204 | 712 | 165 | 177 | 93 | 3e−93 | |
| 6322 | 2.716385262 | down | 0.0000247 | 0.000260774 | BQ627693 | 411 | 18 | 48 | 37 | 1.3 | |
| 6323 | 2.716147248 | down | 0.000000754 | 0.0000286 | AW100760 | 328 | 25 | 48 | 52 | 2e−07 | |
| 6324 | 2.715997882 | down | 0.00000263 | 0.0000607 | BE556216 | 595 | 73 | 166 | 43 | 2e−23 | gravitropism |
| 6325 | 2.71596958 | down | 0.00000821 | 0.000122383 | AW310022 | 650 | 78 | 100 | 78 | 8e−41 | |
| 6326 | 2.715763049 | down | 0.00000183 | 0.000048 | BE556399 | 382 | 61 | 156 | 39 | 6e−20 | protein folding |
| 6327 | 2.714904706 | down | 0.00000304 | 0.0000665 | CD399432 | 390 | 78 | 111 | 70 | 3e−41 | |
| 6328 | 2.714668741 | down | 0.00000819 | 0.00012232 | BM885986 | 404 | 45 | 64 | 70 | 2e−21 | |
| 6329 | 2.714663008 | down | 0.00000487 | 0.0000892 | BG406544 | 461 | 18 | 30 | 60 | 6e−06 | regulation of transcription |
| 6330 | 2.7145157 | down | 0.00000224 | 0.000055 | L23833.1 | | | | | | glutamine phosphoribosylpyrophosphate |
| | | | | | | | | | | | amidotransferase |
| 6331 | 2.714138032 | down | 0.00000213 | 0.000053 | BQ079859 | 522 | 110 | 139 | 79 | 8e−62 | flavonoid biosynthesis |
| 6332 | 2.713425605 | down | 0.0000459 | 0.00040003 | CD411941 | 501 | 23 | 32 | 71 | 2e−08 | |
| 6333 | 2.711645535 | down | 0.000352494 | 0.001702234 | BE820358 | 723 | 86 | 123 | 69 | 3e−41 | protein amino acid phosphorylation |
| 6334 | 2.71079925 | down | 0.000835349 | 0.003198372 | AI443822 | 399 | 117 | 122 | 95 | 3e−61 | ubiquitin-dependent protein catabolism |
| 6335 | 2.710618114 | down | 0.0000038 | 0.0000762 | AI737035 | 425 | 66 | 112 | 58 | 1e−34 | |
| 6336 | 2.710241203 | down | 0.000180902 | 0.001046999 | AW703819 | 257 | 35 | 82 | 42 | 1e−16 | electron transport |
| 6337 | 2.709765674 | down | 0.0000944 | 0.000661623 | CF806755 | 648 | 81 | 209 | 38 | 2e−36 | Putative cytochrome P450, clone CP7, |
| | | | | | | | | | | | electron transport |
| 6338 | 2.709671309 | down | 0.0000096 | 0.000136199 | BG362937 | 487 | 11 | 15 | 73 | 0.044 | |
| 6339 | 2.709050948 | down | 0.00000152 | 0.0000424 | CD408467 | 543 | 47 | 67 | 70 | 1e−22 | |
| 6340 | 2.708101053 | down | 0.00000508 | 0.000092 | AW348709 | 713 | 13 | 40 | 32 | 4.8 | protein folding |
| 6341 | 2.707625338 | down | 0.00000165 | 0.0000446 | CD417226 | 505 | 49 | 75 | 65 | 1e−19 | |
| 6342 | 2.707580746 | down | 0.00000305 | 0.0000665 | BE347455 | 565 | 107 | 189 | 56 | 2e−52 | protein biosynthesis |
| 6343 | 2.706420523 | down | 0.0000882 | 0.000630158 | CA935401 | 315 | 51 | 80 | 63 | 5e−27 | |
| 6344 | 2.705998034 | down | 0.0000519 | 0.000433639 | BU761776 | 602 | 126 | 198 | 63 | 3e−72 | gibberellic acid biosynthesis |
| 6345 | 2.70587952 | down | 0.000186376 | 0.001071266 | T41487 | 429 | 46 | 79 | 58 | 1e−20 | transport |
| 6346 | 2.70542467 | down | 0.00003 | 0.000297721 | AI496452 | 402 | 55 | 64 | 85 | 4e−27 | regulation of transcription\, DNA- |
| | | | | | | | | | | | dependent |
| 6347 | 2.70458497 | down | 0.00000454 | 0.0000853 | BE020273 | 644 | 92 | 178 | 51 | 9e−43 | histidine biosynthesis |
| 6348 | 2.704517515 | down | 0.0000813 | 0.000594673 | BU546955 | 615 | 37 | 68 | 54 | 8e−17 | |
| 6349 | 2.704040439 | down | 0.0000754 | 0.000563529 | CD412855 | 725 | 68 | 127 | 53 | 4e−35 | galactose metabolism |
| 6350 | 2.703433089 | down | 0.0000832 | 0.000603869 | BG046885 | 506 | 80 | 170 | 47 | 2e−31 | brassinosteroid biosynthesis |
| 6351 | 2.70180016 | down | 0.00000227 | 0.0000554 | BE820955 | 503 | 71 | 103 | 68 | 4e−37 | threonine catabolism |
| 6352 | 2.701404619 | down | 0.00000673 | 0.000108581 | BG651885 | 390 | 35 | 120 | 29 | 8e−05 | regulation of transcription |
| 6353 | 2.700267707 | down | 0.00001 | 0.000140916 | BE823646 | 704 | 112 | 177 | 63 | 3e−63 | |
| 6354 | 2.700258328 | down | 0.0000057 | 0.0000984 | BQ253787 | 329 | 9 | 28 | 32 | 3.3 | |
| 6355 | 2.699845204 | down | 0.0000141 | 0.000177559 | CD395311 | 664 | 58 | 79 | 73 | 1e−29 | DNA replication |
| 6356 | 2.699456582 | down | 0.0000381 | 0.000350801 | BE657327 | 833 | 102 | 216 | 47 | 2e−53 | biosynthesis |
| 6357 | 2.699356259 | down | 0.0000109 | 0.000149019 | BQ611816 | 582 | 61 | 68 | 89 | 4e−30 | |
| 6358 | 2.699328571 | down | 0.00000319 | 0.0000683 | CK606530 | 552 | 17 | 47 | 36 | 0.82 | |
| 6359 | 2.699170521 | down | 0.0000306 | 0.000302381 | BQ273100 | 420 | 63 | 99 | 63 | 6e−33 | |
| 6360 | 2.69877067 | down | 0.000193118 | 0.001098265 | BI321901 | 479 | 108 | 158 | 68 | 5e−61 | DNA repair |
| 6361 | 2.698552075 | down | 0.00000286 | 0.0000637 | BI941697 | 360 | 13 | 36 | 36 | 2.6 | |
| 6362 | 2.697010974 | down | 0.00000924 | 0.000132463 | BE608150 | 535 | 10 | 34 | 29 | 6.6 | |
| 6363 | 2.696136651 | down | 0.000133633 | 0.000847205 | AW507708 | 611 | 53 | 98 | 54 | 3e−22 | gravitropism |
| 6364 | 2.694161807 | down | 0.00000252 | 0.0000592 | CD398733 | 306 | 24 | 29 | 82 | 1e−11 | regulation of transcription\, DNA- |
| | | | | | | | | | | | dependent |
| 6365 | 2.69403865 | down | 0.0000526 | 0.000437873 | CD401662 | 589 | 42 | 98 | 42 | 2e−13 | |
| 6366 | 2.693975889 | down | 0.0000117 | 0.000155659 | CD405372 | 667 | 69 | 138 | 50 | 7e−33 | protein amino acid phosphorylation |
| 6367 | 2.693919473 | down | 0.00000208 | 0.0000522 | BI470446 | 537 | 105 | 176 | 59 | 3e−56 | metabolism |
| 6368 | 2.693838959 | down | 0.00000101 | 0.0000337 | BG881621 | 502 | 54 | 132 | 40 | 3e−16 | |
| 6369 | 2.692530996 | down | 0.0000348 | 0.000330641 | CA785545 | 484 | 98 | 161 | 60 | 4e−44 | regulation of transcription\, DNA- |
| | | | | | | | | | | | dependent |
| 6370 | 2.69252043 | down | 0.0000277 | 0.000281408 | BI967166 | 754 | 58 | 94 | 61 | 5e−25 | |
| 6371 | 2.691718386 | down | 0.0000133 | 0.000170634 | BU549789 | 714 | 35 | 129 | 27 | 5e−06 | proteolysis and peptidolysis |
| 6372 | 2.691413597 | down | 0.0000256 | 0.000266882 | CD406617 | 716 | 34 | 40 | 85 | 7e−13 | protein biosynthesis |
| 6373 | 2.690135545 | down | 0.00000271 | 0.0000617 | BM177239 | 550 | 113 | 145 | 77 | 3e−64 | |
| 6374 | 2.688434951 | down | 0.000084 | 0.000608641 | CA799914 | 375 | 22 | 42 | 52 | 3e−09 | regulation of transcription\, DNA- |
| | | | | | | | | | | | dependent |
| 6375 | 2.688108066 | down | 0.0000348 | 0.000330449 | AW831391 | 604 | 28 | 34 | 82 | 5e−10 | galactose metabolism |
| 6376 | 2.687736281 | down | 0.000021 | 0.000231685 | CD391181 | 696 | 121 | 124 | 97 | 2e−66 | microtubule-based process |
| 6377 | 2.687451857 | down | 0.0000174 | 0.000204667 | BI969457 | 752 | 107 | 220 | 48 | 2e−64 | |
| 6378 | 2.687236137 | down | 0.00000654 | 0.000106559 | AW350562 | 739 | 93 | 136 | 68 | 3e−49 | |
| 6379 | 2.686755899 | down | 0.00000117 | 0.0000363 | AW203473 | 370 | 49 | 84 | 58 | 4e−24 | |
| 6380 | 2.686637365 | down | 0.0000635 | 0.000500029 | BU760718 | 642 | 178 | 210 | 84 | e−106 | protein amino acid phosphorylation |
| 6381 | 2.686496953 | down | 0.00000701 | 0.000111253 | BE023321 | 331 | 16 | 34 | 47 | 0.86 | |
| 6382 | 2.68648793 | down | 0.000014 | 0.000176901 | CF806780 | 695 | 154 | 234 | 65 | 6e−90 | carbohydrate metabolism |
| 6383 | 2.686448274 | down | 0.0000198 | 0.000222823 | CF805883 | 671 | 94 | 162 | 58 | 3e−50 | Cytochrome P450-like protein, clone |
| | | | | | | | | | | | CP9, electron transport |
| 6384 | 2.686152986 | down | 0.00000271 | 0.0000617 | BG156576 | 262 | 10 | 37 | 27 | 5.5 | |
| 6385 | 2.686058545 | down | 0.00000202 | 0.0000512 | BQ611849 | 365 | 17 | 25 | 68 | 0.002 | |
| 6386 | 2.68589937 | down | 0.00000197 | 0.0000506 | BE329937 | 454 | 80 | 92 | 86 | 8e−43 | |
| 6387 | 2.68566819 | down | 0.0000181 | 0.000209195 | BU548956 | 543 | 41 | 54 | 75 | 1e−19 | carbohydrate metabolism |
| 6388 | 2.68554065 | down | 0.00000356 | 0.0000731 | BG508253 | 233 | 25 | 55 | 45 | 6e−07 | |
| 6389 | 2.683714033 | down | 0.00000372 | 0.0000753 | AW424105 | 426 | 23 | 70 | 32 | 2.4 | |
| 6390 | 2.68362725 | down | 0.0000271 | 0.000277153 | BI967272 | 744 | 81 | 96 | 84 | 8e−43 | |
| 6391 | 2.683397619 | down | 0.0000104 | 0.000144328 | BI971319 | 778 | 108 | 186 | 58 | 3e−57 | protein amino acid phosphorylation |
| 6392 | 2.683374229 | down | 0.000108987 | 0.000730216 | BI969806 | 653 | 62 | 80 | 77 | 8e−34 | |
| 6393 | 2.683209294 | down | 0.0000252 | 0.000264369 | BU548330 | 665 | 58 | 100 | 58 | 7e−24 | |
| 6394 | 2.683063798 | down | 0.000881407 | 0.003331985 | AI437972 | 385 | 54 | 73 | 73 | 6e−28 | |
| 6395 | 2.682130336 | down | 0.0000013 | 0.0000384 | AW164562 | | | | | | |
| 6396 | 2.682125206 | down | 0.001075427 | 0.003844431 | BG508458 | 259 | 12 | 22 | 54 | 3.2 | biosynthesis |
| 6397 | 2.682124315 | down | 0.000015 | 0.000185652 | CA853861 | 546 | 77 | 174 | 44 | 1e−31 | |
| 6398 | 2.681360401 | down | 0.00000125 | 0.0000375 | BE021189 | 407 | 29 | 118 | 24 | 1.3 | |
| 6399 | 2.681228298 | down | 0.0000421 | 0.000375954 | AF035671.1 | | | | | | arginase |
| 6400 | 2.680832099 | down | 0.00000881 | 0.000128368 | BI972268 | 220 | 9 | 24 | 37 | 4.2 | regulation of transcription\, DNA- |
| | | | | | | | | | | | dependent |
| 6401 | 2.680738417 | down | 0.0000074 | 0.0001153 | AI442425 | 541 | 113 | 181 | 62 | 1e−62 | |
| 6402 | 2.680230429 | down | 0.00000722 | 0.000113545 | AW597829 | 487 | 9 | 25 | 36 | 1.4 | |
| 6403 | 2.679934615 | down | 0.000470382 | 0.002102298 | CD398324 | 582 | 35 | 61 | 57 | 7e−14 | |
| 6404 | 2.679404364 | down | 0.0000101 | 0.000141443 | BM108011 | 601 | 83 | 165 | 50 | 2e−39 | |
| 6405 | 2.67938849 | down | 0.00000417 | 0.0000809 | CD407513 | 712 | 118 | 167 | 70 | 2e−65 | electron transport |
| 6406 | 2.67917781 | down | 0.000648039 | 0.002656714 | CF808851 | 556 | 135 | 159 | 84 | 2e−73 | glycolysis |
| 6407 | 2.678995617 | down | 0.00000317 | 0.0000682 | BI945865 | 495 | 101 | 166 | 60 | 3e−59 | carbohydrate metabolism |
| 6408 | 2.67896791 | down | 0.0000117 | 0.000155659 | BE611098 | 408 | 36 | 55 | 65 | 6e−16 | regulation of transcription |
| 6409 | 2.678741965 | down | 0.000015 | 0.000185595 | BQ298780 | 307 | 15 | 47 | 31 | 1.1 | |
| 6410 | 2.677305641 | down | 0.000126993 | 0.000817791 | BI969295 | 732 | 81 | 154 | 52 | 5e−38 | |
| 6411 | 2.676399726 | down | 0.00000421 | 0.0000813 | BQ629059 | 449 | 72 | 91 | 79 | 3e−40 | transport |
| 6412 | 2.675893897 | down | 0.00000884 | 0.000128725 | BF424240 | 603 | 129 | 180 | 71 | 2e−70 | response to oxidative stress |
| 6413 | 2.674635852 | down | 0.00000129 | 0.0000382 | BG726769 | 421 | 109 | 126 | 86 | 2e−59 | intracellular protein transport |
| 6414 | 2.674433011 | down | 0.000144174 | 0.000892079 | AW348658 | 631 | 32 | 80 | 40 | 9e−13 | protein amino acid phosphorylation |
| 6415 | 2.673669742 | down | 0.00022467 | 0.001227409 | CA819497 | 214 | 11 | 27 | 40 | 1.9 | |
| 6416 | 2.673642618 | down | 0.00000399 | 0.0000785 | BU547879 | 617 | 69 | 121 | 57 | 2e−32 | |
| 6417 | 2.673561436 | down | 0.0000952 | 0.000666043 | BI968104 | 781 | 112 | 159 | 70 | 4e−64 | dicarboxylic acid transport |
| 6418 | 2.673026734 | down | 0.002633328 | 0.007266524 | CF808035 | 556 | 61 | 69 | 88 | 5e−30 | |
| 6419 | 2.671932057 | down | 0.00000181 | 0.0000478 | BE608426 | 456 | 61 | 109 | 55 | 4e−30 | |
| 6420 | 2.671288655 | down | 0.0000016 | 0.000044 | BG839113 | 756 | 122 | 149 | 81 | 3e−68 | |
| 6421 | 2.671179123 | down | 0.0000165 | 0.000197702 | BI321581 | 277 | 75 | 92 | 81 | 2e−39 | L-phenylalanine biosynthesis |
| 6422 | 2.67032807 | down | 0.00000573 | 0.0000986 | CF806255 | 510 | 79 | 96 | 82 | 3e−43 | |
| 6423 | 2.670057735 | down | 0.0000583 | 0.000471182 | BU765847 | 549 | 16 | 54 | 29 | 5.2 | protein folding |
| 6424 | 2.669825541 | down | 0.00000658 | 0.000107059 | AW100503 | 518 | 15 | 40 | 37 | 2.1 | |
| 6425 | 2.669800347 | down | 0.000000783 | 0.0000291 | CD401142 | 384 | 13 | 40 | 32 | 0.28 | metabolism |
| 6426 | 2.669531123 | down | 0.00000537 | 0.0000947 | CD405423 | 579 | 66 | 137 | 48 | 6e−24 | |
| 6427 | 2.668742664 | down | 0.0000741 | 0.000557206 | BI317470 | 455 | 19 | 79 | 24 | 2.1 | |
| 6428 | 2.668483397 | down | 0.000972915 | 0.003583902 | AW350475 | 780 | 120 | 143 | 83 | 1e−67 | water transport |
| 6429 | 2.668364952 | down | 0.0000157 | 0.000191609 | BU544101 | 497 | 73 | 147 | 49 | 1e−33 | |
| 6430 | 2.668318388 | down | 0.00000168 | 0.0000454 | BI967566 | 755 | 53 | 176 | 30 | 2e−15 | cell wall modification |
| 6431 | 2.66820211 | down | 0.000196698 | 0.001113911 | BM309375 | 566 | 115 | 171 | 67 | 3e−56 | protein amino acid phosphorylation |
| 6432 | 2.666555091 | down | 0.000349803 | 0.001692284 | CD405516 | 451 | 94 | 142 | 66 | 4e−49 | |
| 6433 | 2.666474085 | down | 0.00000825 | 0.00012271 | BU080561 | 552 | 132 | 182 | 72 | 2e−73 | proteolysis and peptidolysis |
| 6434 | 2.666356519 | down | 0.00000838 | 0.000124012 | AW350624 | 696 | 80 | 103 | 77 | 4e−39 | nucleosome assembly |
| 6435 | 2.665624248 | down | 0.000224501 | 0.001226667 | BG237865 | 426 | 26 | 65 | 40 | 4e−09 | electron transport |
| 6436 | 2.665291251 | down | 0.00000256 | 0.0000597 | BE659347 | 661 | 123 | 136 | 90 | 3e−69 | |
| 6437 | 2.665198126 | down | 0.00000284 | 0.0000634 | AW100693 | 259 | 13 | 33 | 39 | 4.2 | |
| 6438 | 2.665174195 | down | 0.0000262 | 0.000271118 | BI472159 | 546 | 150 | 153 | 98 | 6e−83 | ethylene mediated signaling pathway |
| 6439 | 2.664686798 | down | 0.00000522 | 0.0000932 | CF807950 | | | | | | |
| 6440 | 2.66391564 | down | 0.00000461 | 0.000086 | BE824387 | 484 | 48 | 92 | 52 | 1e−20 | |
| 6441 | 2.662688445 | down | 0.0000106 | 0.000146441 | BU578491 | 568 | 154 | 189 | 81 | 1e−88 | protein amino acid phosphorylation |
| 6442 | 2.662655439 | down | 0.0000701 | 0.00053606 | CD417784 | 423 | 52 | 56 | 92 | 2e−28 | protein biosynthesis |
| 6443 | 2.662619173 | down | 0.0000516 | 0.000432049 | AI966630 | 511 | 127 | 167 | 76 | 1e−65 | transport |
| 6444 | 2.661666908 | down | 0.00000702 | 0.000111293 | BE020900 | 406 | 42 | 90 | 46 | 1e−15 | regulation of transcription\, DNA- |
| | | | | | | | | | | | dependent |
| 6445 | 2.661622396 | down | 0.00000979 | 0.000138092 | BQ610591 | 446 | 49 | 88 | 55 | 4e−20 | microtubule-based movement |
| 6446 | 2.660264675 | down | 0.00000479 | 0.0000881 | CA936073 | 570 | 68 | 129 | 52 | 1e−32 | |
| 6447 | 2.659409523 | down | 0.00000697 | 0.000110943 | AW310484 | 675 | 74 | 152 | 48 | 5e−31 | protein amino acid phosphorylation |
| 6448 | 2.658818046 | down | 0.00000403 | 0.0000791 | BQ629970 | 549 | 56 | 130 | 43 | 6e−25 | |
| 6449 | 2.658625837 | down | 0.00000376 | 0.0000757 | BU550908 | | | | | | |
| 6450 | 2.658613322 | down | 0.0000174 | 0.000204667 | CF808912 | 556 | 147 | 168 | 87 | 9e−85 | response to light |
| 6451 | 2.658455697 | down | 0.0000306 | 0.000302372 | BM528181 | 560 | 171 | 186 | 91 | 5e−95 | two-component signal transduction |
| | | | | | | | | | | | system (phosphorelay) |
| 6452 | 2.657804637 | down | 0.000709664 | 0.002832782 | M64267.1 | | | | | | Fe-superoxide dismutase |
| 6453 | 2.657569526 | down | 0.0000981 | 0.00067894 | BQ273266 | 550 | 81 | 148 | 54 | 5e−36 | |
| 6454 | 2.657218535 | down | 0.0000777 | 0.000574595 | BM086132 | 549 | 111 | 151 | 73 | 3e−67 | |
| 6455 | 2.656873492 | down | 0.0000068 | 0.000109267 | CD393078 | 575 | 94 | 111 | 84 | 2e−50 | metabolism |
| 6456 | 2.656523205 | down | 0.0000278 | 0.000282355 | BG882585 | 500 | 118 | 164 | 71 | 5e−66 | |
| 6457 | 2.655784432 | down | 0.00000224 | 0.0000549 | AW203482 | 368 | 35 | 43 | 81 | 1e−14 | |
| 6458 | 2.655305762 | down | 0.0000295 | 0.000293854 | BQ610718 | 441 | 19 | 23 | 82 | 2e−05 | |
| 6459 | 2.654091076 | down | 0.000302916 | 0.001528354 | AW203644 | 589 | 128 | 176 | 72 | 6e−74 | |
| 6460 | 2.654018651 | down | 0.000178895 | 0.001039381 | BU548178 | 544 | 55 | 60 | 91 | 4e−26 | auxin polar transport |
| 6461 | 2.653816804 | down | 0.0000119 | 0.000157889 | BQ629820 | 205 | 13 | 25 | 52 | 5.4 | |
| 6462 | 2.653377142 | down | 0.00000307 | 0.0000668 | AW156634 | 425 | 16 | 47 | 34 | 1.8 | phosphatidylcholine biosynthesis |
| 6463 | 2.653303367 | down | 0.00000405 | 0.0000792 | BQ627734 | 591 | 164 | 189 | 86 | 7e−94 | |
| 6464 | 2.653214797 | down | 0.0000182 | 0.000210171 | BE473415 | 463 | 16 | 44 | 36 | 0.44 | |
| 6465 | 2.652236953 | down | 0.000247988 | 0.001320658 | BI892681 | 555 | 113 | 183 | 61 | 2e−68 | |
| 6466 | 2.651281436 | down | 0.000138438 | 0.000868013 | CF808208 | 560 | 121 | 159 | 76 | 1e−62 | |
| 6467 | 2.65039807 | down | 0.00000504 | 0.0000914 | CD410679 | 626 | 12 | 28 | 42 | 5.1 | |
| 6468 | 2.650003919 | down | 0.0000304 | 0.00030097 | BI942877 | 574 | 167 | 188 | 88 | 7e−96 | metabolism |
| 6469 | 2.649863414 | down | 0.000200862 | 0.001131531 | AW620699 | 513 | 77 | 114 | 67 | 2e−43 | aromatic compound metabolism |
| 6470 | 2.6495951 | down | 0.0000189 | 0.000215732 | BI787878 | 497 | 51 | 67 | 76 | 1e−25 | |
| 6471 | 2.649390733 | down | 0.0000155 | 0.000189782 | BG650755 | 186 | 43 | 53 | 81 | 9e−19 | |
| 6472 | 2.649258146 | down | 0.000134546 | 0.0008503 | AI938045 | 529 | 69 | 110 | 62 | 1e−33 | |
| 6473 | 2.648037836 | down | 0.0000127 | 0.000165654 | AI959932 | 489 | 46 | 71 | 64 | 6e−23 | |
| 6474 | 2.646645329 | down | 0.00000452 | 0.0000851 | CA799609 | 408 | 36 | 102 | 35 | 7e−09 | |
| 6475 | 2.646117723 | down | 0.000102869 | 0.000701217 | CD408842 | 516 | 69 | 109 | 63 | 2e−33 | |
| 6476 | 2.646091854 | down | 0.000154091 | 0.000934232 | BI945779 | 252 | 10 | 18 | 55 | 3.3 | carbohydrate metabolism |
| 6477 | 2.645534481 | down | 0.000629125 | 0.002601285 | BE330504 | 537 | 58 | 137 | 42 | 5e−22 | asymmetric cytokinesis |
| 6478 | 2.645466939 | down | 0.000129912 | 0.000830473 | CF809187 | 455 | 106 | 151 | 70 | 2e−55 | protein amino acid phosphorylation |
| 6479 | 2.642854638 | down | 0.00000703 | 0.000111299 | BI470216 | 446 | 17 | 60 | 28 | 0.41 | |
| 6480 | 2.642764277 | down | 0.00014386 | 0.000890955 | T41487 | 429 | 46 | 79 | 58 | 1e−20 | transport |
| 6481 | 2.642461551 | down | 0.000541648 | 0.002333471 | CD397887 | 539 | 54 | 94 | 57 | 2e−22 | amino acid metabolism |
| 6482 | 2.641734717 | down | 0.0000118 | 0.000156922 | BQ094320 | 568 | 44 | 73 | 60 | 2e−16 | |
| 6483 | 2.641491917 | down | 0.00000346 | 0.000072 | CF808452 | 152 | 28 | 34 | 82 | 2e−11 | |
| 6484 | 2.640385834 | down | 0.000122189 | 0.000795299 | BG839300 | 806 | 119 | 171 | 69 | 3e−63 | biosynthesis |
| 6485 | 2.639915247 | down | 0.00000866 | 0.000126797 | BF596692 | 528 | 128 | 176 | 72 | 7e−71 | protein amino acid phosphorylation |
| 6486 | 2.639879613 | down | 0.000252268 | 0.001335889 | BQ453029 | 558 | 58 | 147 | 39 | 5e−24 | electron transport |
| 6487 | 2.639633937 | down | 0.000986798 | 0.003619447 | AB006748.1 | | | | | | Chitinase III-A |
| 6488 | 2.639595366 | down | 0.00000123 | 0.0000372 | CD394842 | 713 | 63 | 149 | 42 | 8e−19 | |
| 6489 | 2.638805988 | down | 0.0000403 | 0.000364077 | CD392748 | 660 | 179 | 211 | 84 | e−104 | photosynthesis light harvesting |
| 6490 | 2.638644129 | down | 0.00000241 | 0.0000576 | BI315808 | 415 | 92 | 118 | 77 | 7e−47 | |
| 6491 | 2.638641054 | down | 0.000108848 | 0.000729806 | BU091633 | 440 | 45 | 100 | 45 | 2e−11 | nucleosome assembly |
| 6492 | 2.638611949 | down | 0.0000165 | 0.000197839 | AI442227 | 385 | 32 | 58 | 55 | 3e−14 | transport |
| 6493 | 2.637691432 | down | 0.00000516 | 0.0000928 | BI699346 | 380 | 13 | 25 | 52 | 0.033 | carbohydrate metabolism |
| 6494 | 2.63768493 | down | 0.00000816 | 0.000122072 | BU544845 | 545 | 61 | 97 | 62 | 7e−26 | |
| 6495 | 2.637251251 | down | 0.00000143 | 0.0000408 | AI988428 | 575 | 19 | 44 | 43 | 2.0 | Hydroxyproline-rich glycoprotein (hrgp) |
| 6496 | 2.636061442 | down | 0.00000342 | 0.0000715 | CD409774 | 409 | 14 | 24 | 58 | 0.013 | wee1 |
| 6497 | 2.636027485 | down | 0.00000161 | 0.000044 | BG044740 | 384 | 72 | 83 | 86 | 1e−40 | |
| 6498 | 2.63602215 | down | 0.00000562 | 0.0000975 | AW705378 | 425 | 16 | 44 | 36 | 4.1 | |
| 6499 | 2.635449675 | down | 0.000398522 | 0.001862805 | CF806136 | 449 | 77 | 113 | 68 | 2e−34 | cell elongation |
| 6500 | 2.635262754 | down | 0.000330601 | 0.001625951 | BF008909 | 476 | 31 | 87 | 35 | 2e−06 | |
| 6501 | 2.635195021 | down | 0.0000181 | 0.000209666 | CA802562 | 734 | 60 | 70 | 85 | 2e−30 | regulation of transcription |
| 6502 | 2.635147669 | down | 0.00000782 | 0.000119065 | BM270808 | 440 | 32 | 40 | 80 | 1e−12 | |
| 6503 | 2.635054339 | down | 0.000177444 | 0.001034132 | BQ453624 | 596 | 129 | 196 | 65 | 1e−74 | |
| 6504 | 2.634840533 | down | 0.000284834 | 0.001461429 | BQ453230 | 517 | 75 | 97 | 77 | 3e−40 | |
| 6505 | 2.634568704 | down | 0.0000104 | 0.000143546 | CF808647 | 566 | 66 | 155 | 42 | 4e−32 | |
| 6506 | 2.634353419 | down | 0.0000502 | 0.000423965 | BQ786753 | 429 | 37 | 52 | 71 | 1e−17 | glycine catabolism |
| 6507 | 2.634102026 | down | 0.00000202 | 0.0000512 | BU548060 | 694 | 28 | 108 | 25 | 1e−04 | |
| 6508 | 2.633666683 | down | 0.00000839 | 0.00012407 | CA936091 | 501 | 45 | 115 | 39 | 4e−20 | protein amino acid phosphorylation |
| 6509 | 2.629017647 | down | 0.00000813 | 0.00012179 | AW100643 | 424 | 43 | 64 | 67 | 2e−21 | |
| 6510 | 2.62815635 | down | 0.000179936 | 0.001042532 | BU761560 | 578 | 144 | 154 | 93 | 8e−77 | |
| 6511 | 2.62802924 | down | 0.0000128 | 0.000166398 | AI441804 | 341 | 81 | 105 | 77 | 5e−43 | carbohydrate biosynthesis |
| 6512 | 2.627243376 | down | 0.00000537 | 0.0000947 | AI856193 | 266 | 12 | 21 | 57 | 0.059 | |
| 6513 | 2.62694831 | down | 0.00000626 | 0.000104576 | CD404616 | 612 | 62 | 73 | 84 | 3e−30 | |
| 6514 | 2.626715965 | down | 0.0000013 | 0.0000384 | BU548512 | 580 | 41 | 114 | 35 | 9e−09 | regulation of transcription\, DNA- |
| | | | | | | | | | | | dependent |
| 6515 | 2.626513319 | down | 0.00000667 | 0.00010807 | BI472174 | 487 | 54 | 72 | 75 | 1e−29 | |
| 6516 | 2.625946297 | down | 0.0000337 | 0.000323531 | CD403681 | 662 | 27 | 59 | 45 | 4e−04 | |
| 6517 | 2.625823849 | down | 0.0000314 | 0.000307891 | BE659709 | 365 | 37 | 66 | 56 | 6e−14 | methylation-dependent chromatin |
| | | | | | | | | | | | silencing |
| 6518 | 2.625590963 | down | 0.0000772 | 0.000572541 | BU761031 | 403 | 20 | 23 | 86 | 1e−04 | regulation of transcription\, DNA- |
| | | | | | | | | | | | dependent |
| 6519 | 2.61371009 | down | 0.000685702 | 0.002762343 | AW203405 | 579 | 156 | 193 | 80 | 5e−91 | transport |
| 6520 | 2.613421381 | down | 0.0000142 | 0.000178829 | CD408042 | 449 | 25 | 96 | 26 | 0.92 | |
| 6521 | 2.613352507 | down | 0.00000327 | 0.0000693 | CF921756 | 676 | 107 | 185 | 57 | 7e−56 | response to oxidative stress |
| 6522 | 2.612301075 | down | 0.000012 | 0.000158522 | BE022356 | 412 | 103 | 137 | 75 | 1e−61 | carbohydrate metabolism |
| 6523 | 2.611803474 | down | 0.00000268 | 0.0000613 | CD400920 | 391 | 12 | 24 | 50 | 0.30 | protein amino acid phosphorylation |
| 6524 | 2.611724869 | down | 0.0000179 | 0.000208232 | AW278764 | 640 | 165 | 213 | 77 | 4e−93 | meristem organization |
| 6525 | 2.610846158 | down | 0.00000181 | 0.0000477 | CD418032 | 425 | 50 | 82 | 60 | 8e−22 | malate metabolism |
| 6526 | 2.610393456 | down | 0.00000746 | 0.000115878 | CF807138 | 384 | 85 | 127 | 66 | 1e−46 | |
| 6527 | 2.610368071 | down | 0.00141966 | 0.004687009 | BE020161 | 549 | 127 | 183 | 69 | 4e−67 | |
| 6528 | 2.610035884 | down | 0.00000116 | 0.0000363 | BE820796 | 771 | 71 | 131 | 54 | 6e−29 | |
| 6529 | 2.609575907 | down | 0.0000065 | 0.000106296 | BM528357 | 541 | 69 | 115 | 60 | 3e−32 | regulation of transcription |
| 6530 | 2.609419414 | down | 0.0000363 | 0.000339449 | CD397377 | 457 | 10 | 21 | 47 | 4.7 | |
| 6531 | 2.609269247 | down | 0.000515181 | 0.00224725 | CF807838 | 665 | 64 | 116 | 55 | 8e−34 | Cv. Williams 82 lipoxygenase |
| 6532 | 2.608865673 | down | 0.000661384 | 0.002695264 | CF808707 | 686 | 136 | 205 | 66 | 1e−72 | protein amino acid phosphorylation |
| 6533 | 2.608490839 | down | 0.00000472 | 0.0000873 | BE822643 | 675 | 133 | 202 | 65 | 1e−75 | carbohydrate metabolism |
| 6534 | 2.608231778 | down | 0.00000283 | 0.0000632 | BE821537 | 356 | 27 | 56 | 48 | 7e−09 | subtilisin-like protease C1, proteolysis |
| | | | | | | | | | | | and peptidolysis |
| 6535 | 2.52726933 | down | 0.00000749 | 0.000116042 | CF807708 | 735 | 147 | 243 | 60 | 4e−80 | proteolysis and peptidolysis |
| 6536 | 2.526859502 | down | 0.000643221 | 0.002643482 | L43921.1 | | | | | | microsomal omega-6 desaturase |
| 6537 | 2.525680967 | down | 0.0000394 | 0.000358219 | BQ742728 | 508 | 41 | 153 | 26 | 1e−08 | |
| 6538 | 2.525655722 | down | 0.00000551 | 0.0000963 | AI440651 | 274 | 55 | 103 | 53 | 8e−25 | protein amino acid phosphorylation |
| 6539 | 2.551725349 | down | 0.0000024 | 0.0000574 | CD393826 | 634 | 121 | 164 | 73 | 2e−69 | |
| 6540 | 2.551618143 | down | 0.0000198 | 0.000222658 | BG155686 | 388 | 64 | 148 | 43 | 4e−25 | |
| 6541 | 2.551014237 | down | 0.0000127 | 0.000165597 | CD406089 | 687 | 72 | 90 | 80 | 2e−35 | photosynthesis |
| 6542 | 2.550660647 | down | 0.0000252 | 0.000264717 | CA935453 | | | | | | |
| 6543 | 2.550542276 | down | 0.000325497 | 0.001608838 | AW394374 | 311 | 12 | 27 | 44 | 4.2 | ubiquitin-dependent protein catabolism |
| 6544 | 2.550227008 | down | 0.00008 | 0.000587909 | BG046111 | 606 | 113 | 170 | 66 | 1e−55 | |
| 6545 | 2.549741247 | down | 0.0000498 | 0.000421812 | BM885300 | 489 | 70 | 149 | 46 | 8e−25 | |
| 6546 | 2.548651144 | down | 0.0000304 | 0.00030067 | AI461081 | 430 | 35 | 93 | 37 | 9e−11 | 34 kDa maturing seed vacuolar thiol |
| | | | | | | | | | | | protease precursor |
| 6547 | 2.548408383 | down | 0.0000142 | 0.00017888 | AW567814 | 463 | 69 | 81 | 85 | 1e−39 | proteolysis and peptidolysis |
| 6548 | 2.54821183 | down | 0.00000649 | 0.000106293 | BQ742661 | 234 | 13 | 24 | 54 | 1.9 | |
| 6549 | 2.548022625 | down | 0.00000448 | 0.0000847 | BI892848 | 571 | 120 | 190 | 63 | 3e−74 | |
| 6550 | 2.547952071 | down | 0.001355263 | 0.004527655 | CF808388 | 633 | 69 | 141 | 48 | 9e−21 | Ethylene responsive protein (EREB). |
| | | | | | | | | | | | regulation of transcription\, DNA- |
| | | | | | | | | | | | dependent |
| 6551 | 2.54780682 | down | 0.0000231 | 0.000248833 | BF324548 | 457 | 45 | 91 | 49 | 2e−23 | |
| 6552 | 2.547615829 | down | 0.0000036 | 0.0000736 | CD416313 | 676 | 79 | 100 | 79 | 2e−40 | response to ethylene stimulus |
| 6553 | 2.547410479 | down | 0.0000109 | 0.000148955 | BG237273 | 231 | 12 | 46 | 26 | 0.38 | tRNA modification |
| 6554 | 2.547052505 | down | 0.00000377 | 0.0000758 | CD403744 | 466 | 45 | 60 | 75 | 3e−22 | transport |
| 6555 | 2.546571597 | down | 0.0000802 | 0.000589138 | AW310322 | 534 | 25 | 36 | 69 | 9e−10 | |
| 6556 | 2.546289879 | down | 0.002086942 | 0.006170759 | CF806136 | 449 | 77 | 113 | 68 | 2e−34 | cell elongation |
| 6557 | 2.546198436 | down | 0.0000393 | 0.00035742 | AW306749 | | | | | | |
| 6558 | 2.54594132 | down | 0.00000795 | 0.00012027 | BM893397 | 463 | 93 | 155 | 60 | 1e−40 | |
| 6559 | 2.545635725 | down | 0.000021 | 0.000231638 | AW100968 | 252 | 11 | 24 | 45 | 1.9 | defense response |
| 6560 | 2.545325607 | down | 0.00000228 | 0.0000554 | BM308028 | 384 | 24 | 50 | 48 | 4e−04 | |
| 6561 | 2.545016785 | down | 0.0000068 | 0.000109267 | AW705533 | 468 | 96 | 158 | 60 | 3e−49 | carbohydrate metabolism |
| 6562 | 2.543640564 | down | 0.0000286 | 0.000288023 | BM954128 | 542 | 100 | 164 | 60 | 1e−51 | protein amino acid phosphorylation |
| 6563 | 2.542551346 | down | 0.00000614 | 0.000102878 | BI787214 | 571 | 33 | 93 | 35 | 3e−05 | metal ion transport |
| 6564 | 2.542540657 | down | 0.000176106 | 0.001030342 | CK606454 | | | | | | |
| 6565 | 2.542258557 | down | 0.0000605 | 0.000482878 | BF424359 | 423 | 32 | 44 | 72 | 2e−14 | |
| 6566 | 2.5418763 | down | 0.000140539 | 0.000876366 | CF921737 | 637 | 112 | 166 | 67 | 9e−58 | protein folding |
| 6567 | 2.541710448 | down | 0.0000317 | 0.000309767 | BE658130 | 853 | 90 | 117 | 76 | 1e−48 | transport |
| 6568 | 2.541438632 | down | 0.0000855 | 0.000616144 | AW705829 | | | | | | |
| 6569 | 2.539894837 | down | 0.000684177 | 0.002758562 | CF807674 | 678 | 183 | 213 | 85 | e−106 | sucrose metabolism |
| 6570 | 2.539732762 | down | 0.000320397 | 0.001590882 | AW310120 | 610 | 71 | 136 | 52 | 1e−34 | |
| 6571 | 2.538003385 | down | 0.0000285 | 0.000287329 | BQ473189 | | | | | | |
| 6572 | 2.53745673 | down | 0.0000106 | 0.000146336 | BI970793 | | | | | | |
| 6573 | 2.536604855 | down | 0.00000446 | 0.0000845 | CD390831 | 532 | 39 | 42 | 92 | 4e−17 | |
| 6574 | 2.536440974 | down | 0.0000533 | 0.000442556 | CA936854 | 447 | 40 | 51 | 78 | 3e−16 | |
| 6575 | 2.53471924 | down | 0.00000396 | 0.0000782 | CA938662 | 620 | 123 | 206 | 59 | 1e−66 | |
| 6576 | 2.53407548 | down | 0.0000115 | 0.000154229 | CD410043 | 690 | 92 | 118 | 77 | 8e−50 | trehalose biosynthesis |
| 6577 | 2.534036799 | down | 0.00000642 | 0.000105745 | AY004243.1 | | | | | | hypothetical LOC547691 |
| 6578 | 2.533937726 | down | 0.0000626 | 0.000494689 | CA935893 | 473 | 61 | 79 | 77 | 3e−34 | |
| 6579 | 2.533745417 | down | 0.0000115 | 0.000154229 | CF808765 | 573 | 171 | 190 | 90 | 2e−97 | Enolase, response to cold |
| 6580 | 2.533295871 | down | 0.0000722 | 0.000546732 | BU551014 | 578 | 44 | 86 | 51 | 5e−14 | |
| 6581 | 2.525196199 | down | 0.0000367 | 0.000342061 | CD391345 | 702 | 45 | 69 | 65 | 1e−23 | |
| 6582 | 2.5251271 | down | 0.0000425 | 0.000378895 | BQ079993 | 543 | 70 | 147 | 47 | 1e−27 | |
| 6583 | 2.52512111 | down | 0.000531731 | 0.002302352 | BM093364 | 490 | 48 | 73 | 65 | 1e−20 | |
| 6584 | 2.524905567 | down | 0.0000102 | 0.000141817 | BU545986 | 536 | 70 | 110 | 63 | 6e−36 | |
| 6585 | 2.520989232 | down | 0.00000789 | 0.000119582 | BI974530 | 462 | 65 | 82 | 79 | 3e−33 | |
| 6586 | 2.52087937 | down | 0.0000482 | 0.000413158 | BF069997 | 498 | 93 | 166 | 56 | 1e−44 | protein amino acid phosphorylation |
| 6587 | 2.520074896 | down | 0.00000587 | 0.000100013 | L23854.1 | | | | | | Nitrate reductase (INR1) /// Inducible |
| | | | | | | | | | | | nitrate reductase 2 (INR2) |
| 6588 | 2.519985442 | down | 0.00000448 | 0.0000847 | BM732317 | 565 | 62 | 106 | 58 | 2e−31 | |
| 6589 | 2.519868817 | down | 0.000164263 | 0.000978708 | BQ741118 | 492 | 84 | 95 | 88 | 9e−46 | |
| 6590 | 2.519766457 | down | 0.000293886 | 0.001495355 | AW349994 | 666 | 90 | 168 | 53 | 3e−52 | galactose metabolism |
| 6591 | 2.519762351 | down | 0.0000333 | 0.000320678 | BE474746 | 529 | 84 | 177 | 47 | 2e−39 | |
| 6592 | 2.519148387 | down | 0.0000129 | 0.000167073 | AW349047 | 599 | 47 | 85 | 55 | 4e−22 | cellulose biosynthesis |
| 6593 | 2.519050417 | down | 0.00000536 | 0.0000947 | CA800744 | 426 | 42 | 65 | 64 | 8e−20 | |
| 6594 | 2.51823469 | down | 0.000096 | 0.000670438 | BM093903 | 272 | 10 | 24 | 41 | 9.3 | |
| 6595 | 2.518184919 | down | 0.00000214 | 0.0000533 | AW309951 | 447 | 18 | 57 | 31 | 0.31 | |
| 6596 | 2.608015673 | down | 0.0000152 | 0.00018777 | AI440556 | 422 | 80 | 137 | 58 | 2e−44 | DNA replication\, priming |
| 6597 | 2.607810902 | down | 0.00000284 | 0.0000633 | CA802301 | 683 | 207 | 220 | 94 | e−118 | |
| 6598 | 2.607700089 | down | 0.00000101 | 0.0000337 | AI494929 | 416 | 90 | 138 | 65 | 2e−47 | metabolism |
| 6599 | 2.607631003 | down | 0.000203928 | 0.001143833 | AI442846 | | | | | | |
| 6600 | 2.607513465 | down | 0.00000457 | 0.0000856 | AF243372.1 | | | | | | glutathione S-transferase GST 17 |
| 6601 | 2.607151765 | down | 0.00000423 | 0.0000816 | BI973058 | 595 | 135 | 147 | 91 | 5e−77 | |
| 6602 | 2.606758585 | down | 0.0000114 | 0.000153343 | BI969004 | 706 | 54 | 93 | 58 | 7e−26 | |
| 6603 | 2.606199363 | down | 0.0000913 | 0.000646961 | AI440894 | 334 | 46 | 107 | 42 | 4e−15 | protein amino acid phosphorylation |
| 6604 | 2.605262646 | down | 0.0000155 | 0.000189782 | BI969224 | 766 | 88 | 98 | 89 | 7e−43 | |
| 6605 | 2.604785702 | down | 0.0000385 | 0.000352804 | AI437780 | 445 | 91 | 156 | 58 | 3e−47 | protein amino acid phosphorylation |
| 6606 | 2.604485089 | down | 0.00000879 | 0.000128216 | BE331101 | 666 | 162 | 221 | 73 | 1e−97 | metabolism |
| 6607 | 2.604331165 | down | 0.00014301 | 0.000888094 | BF598602 | 587 | 104 | 181 | 57 | 2e−49 | protein amino acid phosphorylation |
| 6608 | 2.604162347 | down | 0.00000259 | 0.0000601 | AI988066 | 536 | 53 | 92 | 57 | 5e−17 | |
| 6609 | 2.60399686 | down | 0.0000383 | 0.000352083 | BG044001 | 555 | 166 | 185 | 89 | 4e−97 | protein amino acid phosphorylation |
| 6610 | 2.603700919 | down | 0.0000192 | 0.000218319 | CD394831 | 261 | 15 | 47 | 31 | 2.5 | galactose metabolism |
| 6611 | 2.603667448 | down | 0.0000163 | 0.000196399 | AW755458 | 510 | 116 | 169 | 68 | 8e−64 | protein amino acid phosphorylation |
| 6612 | 2.6027785 | down | 0.0000148 | 0.000183754 | CA800068 | 740 | 118 | 211 | 55 | 8e−62 | |
| 6613 | 2.602649694 | down | 0.0000362 | 0.000338603 | BE331305 | 474 | 28 | 47 | 59 | 8e−09 | transport |
| 6614 | 2.602340671 | down | 0.00000952 | 0.000135375 | BM188517 | 557 | 156 | 185 | 84 | 8e−89 | |
| 6615 | 2.601946955 | down | 0.00000357 | 0.0000732 | AW119808 | 613 | 73 | 219 | 33 | 3e−22 | |
| 6616 | 2.601843465 | down | 0.00000291 | 0.0000644 | BG790368 | 323 | 57 | 98 | 58 | 9e−30 | metabolism |
| 6617 | 2.601755848 | down | 0.00000519 | 0.000093 | CD417050 | 605 | 55 | 99 | 55 | 3e−26 | transport |
| 6618 | 2.601622596 | down | 0.00000275 | 0.0000621 | BI968943 | 783 | 85 | 144 | 59 | 6e−39 | |
| 6619 | 2.599436897 | down | 0.0000017 | 0.0000457 | BF008941 | 447 | 84 | 133 | 63 | 7e−41 | DNA methylation |
| 6620 | 2.599328439 | down | 0.000167091 | 0.000992256 | AW432320 | 472 | 44 | 122 | 36 | 3e−10 | regulation of transcription\, DNA- |
| | | | | | | | | | | | dependent |
| 6621 | 2.598572667 | down | 0.00000145 | 0.0000413 | AW459395 | 390 | 23 | 52 | 44 | 2e−04 | |
| 6622 | 2.598116299 | down | 0.000145249 | 0.000896523 | BI785285 | 525 | 88 | 165 | 53 | 6e−41 | proteolysis and peptidolysis |
| 6623 | 2.598063818 | down | 0.0000052 | 0.0000931 | BQ452585 | 408 | 21 | 26 | 80 | 9e−07 | |
| 6624 | 2.597948924 | down | 0.0000102 | 0.000142275 | BQ299032 | 290 | 14 | 47 | 29 | 9.2 | RNA dependent DNA replication |
| 6625 | 2.597901442 | down | 0.0000277 | 0.000281287 | BE820941 | 605 | 31 | 135 | 22 | 7e−04 | |
| 6626 | 2.597715134 | down | 0.0000323 | 0.000313969 | CA937659 | 466 | 70 | 89 | 78 | 9e−37 | glycolipid biosynthesis |
| 6627 | 2.596927645 | down | 0.000123581 | 0.000802134 | BU549195 | 695 | 121 | 165 | 73 | 6e−69 | protein amino acid phosphorylation |
| 6628 | 2.596747556 | down | 0.00000266 | 0.000061 | CF808081 | 538 | 114 | 153 | 74 | 2e−65 | carbohydrate metabolism |
| 6629 | 2.596219284 | down | 0.0000326 | 0.000315668 | AW277349 | 463 | 93 | 154 | 60 | 1e−47 | transport |
| 6630 | 2.596057219 | down | 0.0000584 | 0.000471799 | AW760011 | 623 | 140 | 206 | 67 | 3e−73 | signal transduction |
| 6631 | 2.595667284 | down | 0.0000016 | 0.000044 | BI968074 | 776 | 90 | 180 | 50 | 1e−41 | amino acid biosynthesis |
| 6632 | 2.595269004 | down | 0.00000466 | 0.0000867 | BU762711 | 428 | 63 | 94 | 67 | 3e−31 | proteolysis and peptidolysis |
| 6633 | 2.595075913 | down | 0.0000474 | 0.000408527 | BU551224 | 661 | 11 | 20 | 55 | 1.1 | |
| 6634 | 2.595033145 | down | 0.0000356 | 0.000334672 | BE023631 | 479 | 39 | 61 | 63 | 1e−16 | metabolism |
| 6635 | 2.594938535 | down | 0.000359732 | 0.001728311 | AW666282 | 571 | 145 | 189 | 76 | 2e−77 | |
| 6636 | 2.594825821 | down | 0.000130181 | 0.000831487 | BU549952 | 683 | 87 | 167 | 52 | 3e−43 | oligopeptide transport |
| 6637 | 2.594430677 | down | 0.00000712 | 0.000112458 | BU083085 | 576 | 33 | 113 | 29 | 4e−04 | |
| 6638 | 2.593643071 | down | 0.0000019 | 0.0000493 | CD404113 | 671 | 115 | 173 | 66 | 8e−63 | |
| 6639 | 2.59322189 | down | 0.00000122 | 0.0000371 | CD406446 | 490 | 17 | 48 | 35 | 2.5 | |
| 6640 | 2.593081913 | down | 0.00000574 | 0.0000986 | CA935084 | 483 | 33 | 38 | 86 | 2e−13 | proteolysis and peptidolysis |
| 6641 | 2.592812469 | down | 0.000000999 | 0.0000336 | BQ252812 | 432 | 58 | 91 | 63 | 2e−32 | regulation of transcription\, DNA- |
| | | | | | | | | | | | dependent |
| 6642 | 2.592204161 | down | 0.00000321 | 0.0000686 | CF808593 | 558 | 148 | 149 | 99 | 5e−81 | calmodulin, calcium ion sensing |
| 6643 | 2.591986637 | down | 0.00000258 | 0.0000599 | BG362795 | 547 | 94 | 160 | 58 | 5e−48 | |
| 6644 | 2.590834184 | down | 0.0000288 | 0.000288977 | BG359602 | 500 | 95 | 154 | 61 | 6e−56 | |
| 6645 | 2.589573645 | down | 0.0000223 | 0.000242049 | BQ740933 | 599 | 122 | 208 | 58 | 9e−57 | ciliary/flagellar motility |
| 6646 | 2.589020118 | down | 0.00000361 | 0.0000737 | AI442775 | 529 | 67 | 83 | 80 | 2e−34 | protein amino acid dephosphorylation |
| 6647 | 2.588742586 | down | 0.000041 | 0.00036898 | BF325442 | 492 | 132 | 164 | 80 | 3e−78 | organogenesis |
| 6648 | 2.588664714 | down | 0.00000132 | 0.0000387 | CD397233 | 645 | 37 | 53 | 69 | 2e−13 | lipid metabolism |
| 6649 | 2.58829938 | down | 0.000057 | 0.000464816 | BI785751 | 558 | 155 | 170 | 91 | 3e−86 | proliferating cell nuclear antigen, |
| | | | | | | | | | | | regulation of DNA replication |
| 6650 | 2.588144449 | down | 0.00000445 | 0.0000843 | CF807379 | 656 | 180 | 205 | 87 | e−108 | potassium ion transport |
| 6651 | 2.587567106 | down | 0.0000263 | 0.000272262 | BE806704 | 452 | 67 | 164 | 40 | 1e−20 | |
| 6652 | 2.587212304 | down | 0.00006 | 0.000480313 | BU544060 | 517 | 29 | 108 | 26 | 0.32 | |
| 6653 | 2.587146879 | down | 0.00000861 | 0.000126391 | CF808742 | 556 | 118 | 185 | 63 | 5e−69 | proteolysis and peptidolysis |
| 6654 | 2.587035457 | down | 0.0000205 | 0.000227762 | BU548719 | 631 | 43 | 134 | 32 | 2e−13 | |
| 6655 | 2.586902633 | down | 0.000283347 | 0.001456382 | BE658510 | 545 | 59 | 75 | 78 | 6e−28 | cell fate specification |
| 6656 | 2.586540554 | down | 0.0000707 | 0.000539585 | BU546906 | 557 | 81 | 175 | 46 | 6e−44 | |
| 6657 | 2.585949224 | down | 0.00000522 | 0.0000932 | BQ786355 | 150 | 11 | 32 | 34 | 9.2 | |
| 6658 | 2.585578443 | down | 0.000029 | 0.000290828 | BG882474 | 426 | 12 | 35 | 34 | 1.4 | |
| 6659 | 2.585557182 | down | 0.00000131 | 0.0000384 | AW567698 | 360 | 24 | 56 | 42 | 8e−10 | transport |
| 6660 | 2.585329867 | down | 0.00000729 | 0.000114115 | AW278809 | 743 | 160 | 245 | 65 | 2e−89 | electron transport |
| 6661 | 2.584975606 | down | 0.00123675 | 0.004243733 | BG551036 | 474 | 58 | 146 | 39 | 3e−14 | |
| 6662 | 2.584904231 | down | 0.00000397 | 0.0000783 | AW309940 | 557 | 48 | 116 | 41 | 4e−18 | |
| 6663 | 2.584776944 | down | 0.000162847 | 0.000973509 | CA936417 | 586 | 85 | 140 | 60 | 4e−47 | |
| 6664 | 2.584191349 | down | 0.00000858 | 0.000126018 | BQ741663 | 592 | 94 | 181 | 51 | 5e−47 | response to pathogenic fungi |
| 6665 | 2.583284528 | down | 0.0000346 | 0.000329218 | BG157397 | 383 | 9 | 24 | 37 | 5.3 | |
| 6666 | 2.583103517 | down | 0.00000503 | 0.0000912 | BU764968 | 485 | 89 | 119 | 74 | 9e−46 | response to pathogenic fungi |
| 6667 | 2.582908841 | down | 0.0000206 | 0.000228841 | CD415224 | 697 | 113 | 144 | 78 | 1e−60 | |
| 6668 | 2.582822731 | down | 0.0000165 | 0.000197839 | BE661506 | 783 | 94 | 170 | 55 | 3e−48 | |
| 6669 | 2.58271771 | down | 0.0000072 | 0.000113242 | BQ452873 | 540 | 37 | 52 | 71 | 5e−14 | protein amino acid dephosphorylation |
| 6670 | 2.582389427 | down | 0.000302516 | 0.001526951 | BI785748 | 577 | 163 | 187 | 87 | 1e−99 | |
| 6671 | 2.582215057 | down | 0.0000562 | 0.000460188 | BM525082 | 571 | 120 | 192 | 62 | 3e−68 | |
| 6672 | 2.582182216 | down | 0.000208422 | 0.001161939 | BE329562 | 531 | 95 | 147 | 64 | 6e−51 | proteolysis and peptidolysis |
| 6673 | 2.581818138 | down | 0.00000276 | 0.0000622 | BU765544 | 426 | 40 | 61 | 65 | 1e−18 | L-serine biosynthesis |
| 6674 | 2.581511688 | down | 0.0000147 | 0.000183603 | AW704921 | 411 | 47 | 91 | 51 | 1e−18 | |
| 6675 | 2.580841571 | down | 0.00000405 | 0.0000792 | AW350843 | 597 | 45 | 76 | 59 | 9e−23 | |
| 6676 | 2.580471852 | down | 0.00000544 | 0.0000955 | BM528250 | 555 | 81 | 145 | 55 | 6e−38 | signal transduction |
| 6677 | 2.579950195 | down | 0.0000712 | 0.000541612 | AW309938 | 676 | 87 | 127 | 68 | 1e−44 | |
| 6678 | 2.578780065 | down | 0.00000167 | 0.0000451 | BI425084 | 503 | 41 | 52 | 78 | 2e−22 | |
| 6679 | 2.578076735 | down | 0.00000393 | 0.000078 | CD410800 | 579 | 57 | 93 | 61 | 3e−27 | |
| 6680 | 2.578047536 | down | 0.000127089 | 0.00081799 | BI784930 | 468 | 24 | 38 | 63 | 6e−09 | protein amino acid phosphorylation |
| 6681 | 2.577766965 | down | 0.001890617 | 0.005753066 | BF424596 | 334 | 101 | 111 | 90 | 2e−53 | DNA repair |
| 6682 | 2.577676066 | down | 0.000956517 | 0.003538398 | AW459535 | 399 | 16 | 39 | 41 | 0.082 | |
| 6683 | 2.57742504 | down | 0.0000826 | 0.000601333 | CD396327 | 306 | 23 | 49 | 46 | 2e−06 | metabolism |
| 6684 | 2.577406827 | down | 0.001771251 | 0.005496109 | CA820138 | 420 | 18 | 20 | 90 | 0.001 | response to auxin stimulus |
| 6685 | 2.576897288 | down | 0.00002 | 0.000223716 | BI316139 | 358 | 61 | 91 | 67 | 4e−31 | cellulose biosynthesis |
| 6686 | 2.576401175 | down | 0.0000119 | 0.00015762 | BE607642 | 405 | 13 | 19 | 68 | 0.023 | protein amino acid phosphorylation |
| 6687 | 2.575898543 | down | 0.0000455 | 0.000397753 | BI972608 | 552 | 112 | 136 | 82 | 7e−64 | proteolysis and peptidolysis |
| 6688 | 2.57580862 | down | 0.00000217 | 0.0000536 | CA782170 | 707 | 137 | 170 | 80 | 1e−79 | |
| 6689 | 2.575227978 | down | 0.00000635 | 0.000105046 | BQ740358 | 600 | 132 | 197 | 67 | 5e−74 | protein amino acid phosphorylation |
| 6690 | 2.575059288 | down | 0.00000973 | 0.0001376 | BU082861 | 584 | 70 | 95 | 73 | 8e−40 | regulation of transcription\, DNA- |
| | | | | | | | | | | | dependent |
| 6691 | 2.574452046 | down | 0.000266764 | 0.001392072 | AW432588 | 507 | 32 | 72 | 44 | 2e−11 | NAD(P)H dependent 6′-deoxychalcone |
| | | | | | | | | | | | synthase, potassium ion transport |
| 6692 | 2.574094252 | down | 0.00000717 | 0.000113023 | CF807468 | 567 | 105 | 151 | 69 | 2e−56 | |
| 6693 | 2.573837184 | down | 0.0000157 | 0.000191236 | BE658641 | 638 | 15 | 50 | 30 | 2.4 | |
| 6694 | 2.57378899 | down | 0.0000413 | 0.000370948 | BU762960 | 364 | 13 | 33 | 39 | 6.0 | |
| 6695 | 2.573239422 | down | 0.000203863 | 0.00114364 | BU765246 | 521 | 26 | 55 | 47 | 6e−06 | |
| 6696 | 2.572859743 | down | 0.0000542 | 0.000447122 | AW666190 | 464 | 105 | 151 | 69 | 4e−59 | |
| 6697 | 2.572430022 | down | 0.002292371 | 0.006591137 | BF009589 | 400 | 91 | 129 | 70 | 7e−54 | proteolysis and peptidolysis |
| 6698 | 2.571569774 | down | 0.00000258 | 0.00006 | BU760781 | 307 | 11 | 27 | 40 | 4.2 | |
| 6699 | 2.570641988 | down | 0.0000101 | 0.000141229 | CD403992 | 563 | 39 | 73 | 53 | 7e−13 | |
| 6700 | 2.570318477 | down | 0.0000086 | 0.000126339 | CA850872 | 344 | 31 | 59 | 52 | 3e−10 | protein amino acid phosphorylation |
| 6701 | 2.57009176 | down | 0.00000139 | 0.0000402 | BI969322 | 701 | 39 | 44 | 88 | 4e−21 | regulation of transcription |
| 6702 | 2.570075812 | down | 0.0000703 | 0.000537148 | BI969302 | 750 | 68 | 178 | 38 | 7e−34 | |
| 6703 | 2.569875425 | down | 0.000179071 | 0.001039602 | BI787925 | 570 | 111 | 188 | 59 | 6e−60 | |
| 6704 | 2.569601371 | down | 0.00000475 | 0.0000878 | CA936241 | 517 | 45 | 75 | 60 | 3e−17 | ion transport |
| 6705 | 2.569463573 | down | 0.00000271 | 0.0000618 | AW132226 | 296 | 12 | 33 | 36 | 9.4 | |
| 6706 | 2.569411575 | down | 0.0000202 | 0.000224994 | CA800956 | 658 | 12 | 34 | 35 | 9.4 | |
| 6707 | 2.569104885 | down | 0.00000605 | 0.000101975 | CD418592 | 676 | 92 | 96 | 95 | 2e−45 | nucleosome assembly |
| 6708 | 2.568669802 | down | 0.0000652 | 0.000508891 | BQ081195 | 485 | 76 | 110 | 69 | 2e−40 | regulation of transcription |
| 6709 | 2.568517467 | down | 0.0000179 | 0.000207836 | CA800792 | 610 | 186 | 203 | 91 | e−105 | cellulose biosynthesis |
| 6710 | 2.568486024 | down | 0.0000142 | 0.000179016 | CD400159 | 595 | 15 | 33 | 45 | 4.6 | |
| 6711 | 2.568343649 | down | 0.0000706 | 0.000539414 | BU082029 | 422 | 11 | 22 | 50 | 4.0 | electron transport |
| 6712 | 2.568280563 | down | 0.0000385 | 0.000352804 | BE819952 | 763 | 23 | 52 | 44 | 5e−06 | apoptosis |
| 6713 | 2.568136433 | down | 0.000001 | 0.0000337 | CD404912 | 710 | 60 | 92 | 65 | 3e−25 | ubiquitin-dependent protein catabolism |
| 6714 | 2.56811341 | down | 0.00000153 | 0.0000426 | X62303.1 | | | | | | mitotic cyclin |
| 6715 | 2.567770074 | down | 0.00000405 | 0.0000792 | BQ611599 | 418 | 15 | 57 | 26 | 1.0 | cation transport |
| 6716 | 2.567663519 | down | 0.000176474 | 0.001031225 | CF809057 | 788 | 152 | 210 | 72 | 2e−94 | |
| 6717 | 2.566907653 | down | 0.00000163 | 0.0000444 | CA803108 | 574 | 42 | 75 | 56 | 3e−16 | |
| 6718 | 2.566834745 | down | 0.00000239 | 0.0000574 | BI968720 | 381 | 99 | 116 | 85 | 1e−53 | L-phenylalanine biosynthesis |
| 6719 | 2.566001127 | down | 0.00000329 | 0.0000696 | BM085247 | 591 | 121 | 199 | 60 | 6e−61 | carbohydrate metabolism |
| 6720 | 2.565997213 | down | 0.00000502 | 0.0000912 | AI461103 | 521 | 59 | 93 | 63 | 5e−27 | response to auxin stimulus |
| 6721 | 2.565956624 | down | 0.0000493 | 0.000418849 | BI893588 | 604 | 75 | 201 | 37 | 7e−34 | protein amino acid phosphorylation |
| 6722 | 2.565039811 | down | 0.00000555 | 0.0000967 | CD391759 | 606 | 39 | 74 | 52 | 1e−15 | protein amino acid phosphorylation |
| 6723 | 2.565031581 | down | 0.0000218 | 0.000238448 | BI470028 | 543 | 82 | 148 | 55 | 3e−42 | |
| 6724 | 2.564981301 | down | 0.00007 | 0.00053543 | AW310615 | 616 | 56 | 127 | 44 | 4e−20 | protein amino acid phosphorylation |
| 6725 | 2.564010041 | down | 0.0000592 | 0.000476067 | CF807857 | 614 | 162 | 183 | 88 | 5e−94 | |
| 6726 | 2.563305898 | down | 0.0000564 | 0.000460872 | AW310451 | 648 | 46 | 137 | 33 | 2e−12 | |
| 6727 | 2.56322809 | down | 0.003241994 | 0.008408913 | CA784156 | 507 | 18 | 44 | 40 | 2e−04 | |
| 6728 | 2.562820418 | down | 0.000182005 | 0.001053058 | CF807488 | 678 | 135 | 160 | 84 | 1e−79 | carbohydrate metabolism |
| 6729 | 2.562340966 | down | 0.0000267 | 0.000274684 | BI970516 | 792 | 115 | 168 | 68 | 3e−57 | |
| 6730 | 2.562091635 | down | 0.00000486 | 0.0000891 | BI321141 | 571 | 41 | 64 | 64 | 7e−16 | protein biosynthesis |
| 6731 | 2.561913825 | down | 0.0000384 | 0.000352245 | BI893280 | 576 | 91 | 145 | 62 | 4e−47 | |
| 6732 | 2.561853586 | down | 0.00000357 | 0.0000732 | BM307407 | 422 | 11 | 34 | 32 | 4.0 | |
| 6733 | 2.561636702 | down | 0.000000957 | 0.0000327 | AY204712.1 | | | | | | microsomal omega-3 fatty acid |
| | | | | | | | | | | | desaturase |
| 6734 | 2.561221662 | down | 0.00000307 | 0.0000669 | BU964441 | 607 | 168 | 200 | 84 | 2e−94 | protein amino acid phosphorylation |
| 6735 | 2.560007142 | down | 0.0000723 | 0.000547008 | BI426425 | 518 | 156 | 172 | 90 | 6e−96 | |
| 6736 | 2.558856404 | down | 0.00000135 | 0.0000395 | BI970989 | 788 | 139 | 184 | 75 | 3e−75 | amino acid metabolism |
| 6737 | 2.558365298 | down | 0.000129316 | 0.000827507 | CD394551 | 690 | 27 | 87 | 31 | 0.013 | |
| 6738 | 2.55824875 | down | 0.0000686 | 0.000527954 | BG362899 | 578 | 99 | 124 | 79 | 3e−57 | |
| 6739 | 2.558216809 | down | 0.00000696 | 0.000110881 | CK606384 | 885 | 124 | 198 | 62 | 9e−56 | ossification |
| 6740 | 2.557684732 | down | 0.00000255 | 0.0000596 | AF243368.1 | | | | | | glutathione S-transferase GST 13 |
| 6741 | 2.557092328 | down | 0.0000365 | 0.000341416 | BG237049 | | | | | | |
| 6742 | 2.557048617 | down | 0.0000124 | 0.000162788 | AW186376 | 487 | 94 | 129 | 72 | 3e−47 | |
| 6743 | 2.55671758 | down | 0.000484595 | 0.002149725 | CA801928 | 601 | 66 | 115 | 57 | 6e−27 | |
| 6744 | 2.556447396 | down | 0.00000402 | 0.000079 | CD416009 | 224 | 43 | 77 | 55 | 2e−16 | |
| 6745 | 2.556320501 | down | 0.00000554 | 0.0000966 | CA851541 | 353 | 22 | 75 | 29 | 4.3 | regulation of transcription\, DNA- |
| | | | | | | | | | | | dependent |
| 6746 | 2.555976775 | down | 0.00000681 | 0.000109267 | BG041917 | 501 | 19 | 41 | 46 | 0.006 | regulation of transcription\, DNA- |
| | | | | | | | | | | | dependent |
| 6747 | 2.555925104 | down | 0.0000653 | 0.000509425 | AW831345 | 360 | 94 | 116 | 81 | 3e−49 | carbohydrate metabolism |
| 6748 | 2.554979018 | down | 0.000635843 | 0.002621858 | BG881629 | 473 | 115 | 156 | 73 | 4e−70 | proteolysis and peptidolysis |
| 6749 | 2.55433082 | down | 0.000148131 | 0.000908201 | BI967418 | | | | | | |
| 6750 | 2.554324557 | down | 0.0000412 | 0.000369942 | AW101190 | 337 | 17 | 20 | 85 | 3e−05 | regulation of transcription\, DNA- |
| | | | | | | | | | | | dependent |
| 6751 | 2.554064749 | down | 0.00000695 | 0.000110807 | BI971152 | 779 | 54 | 103 | 52 | 1e−20 | response to light |
| 6752 | 2.553838198 | down | 0.000138983 | 0.000870267 | CF808901 | 561 | 155 | 180 | 86 | 6e−95 | expansin |
| 6753 | 2.552847817 | down | 0.0000101 | 0.000141229 | BM731619 | 545 | 144 | 151 | 95 | 1e−79 | |
| 6754 | 2.55259077 | down | 0.0000103 | 0.000142633 | CF920340 | 715 | 120 | 160 | 75 | 5e−68 | |
| 6755 | 2.532881295 | down | 0.0000859 | 0.000618014 | CD399348 | 554 | 14 | 37 | 37 | 0.37 | regulation of transcription\, DNA- |
| | | | | | | | | | | | dependent |
| 6756 | 2.532706797 | down | 0.0000435 | 0.000385435 | AI437666 | 428 | 109 | 141 | 77 | 3e−63 | |
| 6757 | 2.532245899 | down | 0.000372037 | 0.00177204 | BE803852 | 480 | 93 | 162 | 57 | 2e−45 | galactose metabolism |
| 6758 | 2.53221006 | down | 0.000464096 | 0.002081127 | BQ452843 | 288 | 12 | 34 | 35 | 1.1 | |
| 6759 | 2.531277448 | down | 0.0000102 | 0.000141817 | BQ628642 | 283 | 17 | 19 | 89 | 0.004 | regulation of cell cycle |
| 6760 | 2.531234669 | down | 0.0000076 | 0.000117138 | BI969660 | 671 | 79 | 111 | 71 | 3e−41 | methionine metabolism |
| 6761 | 2.531204988 | down | 0.00000558 | 0.000097 | CD417606 | 670 | 77 | 103 | 74 | 8e−42 | metabolism |
| 6762 | 2.530534623 | down | 0.0000695 | 0.000532936 | CA820596 | 519 | 13 | 36 | 36 | 2.1 | |
| 6763 | 2.530084668 | down | 0.0000153 | 0.000188485 | BE820839 | 538 | 49 | 68 | 72 | 3e−25 | |
| 6764 | 2.529873195 | down | 0.00000518 | 0.0000929 | BG511742 | 357 | 15 | 59 | 25 | 0.51 | |
| 6765 | 2.529120318 | down | 0.00000558 | 0.000097 | BQ296321 | 420 | 28 | 80 | 35 | 0.032 | |
| 6766 | 2.529081062 | down | 0.0000604 | 0.000482145 | BQ296146 | 502 | 109 | 167 | 65 | 2e−56 | |
| 6767 | 2.529029274 | down | 0.0000115 | 0.000154229 | BU549900 | 516 | 31 | 47 | 65 | 2e−11 | |
| 6768 | 2.528970512 | down | 0.00000211 | 0.0000528 | BG511824 | 338 | 16 | 21 | 76 | 0.035 | |
| 6769 | 2.52860878 | down | 0.0000995 | 0.000685621 | AI965964 | 421 | 122 | 183 | 66 | 6e−60 | |
| 6770 | 2.528539928 | down | 0.0000189 | 0.000216053 | BI970181 | 685 | 105 | 142 | 73 | 4e−56 | protein amino acid phosphorylation |
| 6771 | 2.528341376 | down | 0.00000838 | 0.000124012 | CD408888 | 597 | 26 | 57 | 45 | 9e−07 | regulation of transcription\, DNA- |
| | | | | | | | | | | | dependent |
| 6772 | 2.528332499 | down | 0.0000313 | 0.000307454 | AW310724 | 430 | 46 | 80 | 57 | 1e−18 | |
| 6773 | 2.528279801 | down | 0.000302036 | 0.001525349 | BU550913 | 663 | 73 | 103 | 70 | 5e−39 | transport |
| 6774 | 2.528178806 | down | 0.0000767 | 0.000569861 | BU545170 | 589 | 66 | 73 | 90 | 2e−36 | |
| 6775 | 2.527943857 | down | 0.00000467 | 0.0000869 | CA799585 | 435 | 93 | 132 | 70 | 5e−50 | regulation of transcription\, DNA- |
| | | | | | | | | | | | dependent |
| 6776 | 2.527676561 | down | 0.00000199 | 0.0000509 | BI320672 | | | | | | |
| 6777 | 2.527675402 | down | 0.000932708 | 0.003475089 | AW200709 | 555 | 111 | 159 | 69 | 5e−61 | |
| 6778 | 2.527656502 | down | 0.0000846 | 0.000611489 | BE210185 | 570 | 158 | 205 | 77 | 1e−85 | |
| 6779 | 2.52746261 | down | 0.0000565 | 0.000461313 | AW348887 | 732 | 98 | 164 | 59 | 4e−45 | regulation of transcription |
| 6780 | 2.527424894 | down | 0.00000317 | 0.0000682 | BQ452927 | 304 | 11 | 30 | 36 | 3.2 | |
| 6781 | 2.51811471 | down | 0.00000141 | 0.0000405 | BM527927 | 549 | 100 | 151 | 66 | 5e−54 | |
| 6782 | 2.517994402 | down | 0.000196198 | 0.001111247 | CF806504 | 512 | 109 | 131 | 83 | 6e−61 | |
| 6783 | 2.517587817 | down | 0.0000628 | 0.000495778 | BQ741884 | 622 | 145 | 205 | 70 | 8e−83 | |
| 6784 | 2.517479151 | down | 0.00055146 | 0.002365351 | CF808358 | 653 | 166 | 219 | 75 | 3e−90 | GAI1, regulation of transcription |
| 6785 | 2.516480182 | down | 0.00000762 | 0.000117192 | CD415059 | 685 | 69 | 100 | 69 | 1e−42 | electron transport |
| 6786 | 2.515882534 | down | 0.00000312 | 0.0000676 | AW203376 | 456 | 54 | 121 | 44 | 6e−25 | |
| 6787 | 2.515623616 | down | 0.00000653 | 0.000106503 | BG044600 | 255 | 68 | 85 | 80 | 4e−34 | small GTPase mediated signal |
| | | | | | | | | | | | transduction |
| 6788 | 2.514902172 | down | 0.00000273 | 0.0000619 | BQ080184 | 535 | 109 | 181 | 60 | 1e−53 | |
| 6789 | 2.514521319 | down | 0.00000522 | 0.0000932 | BE330250 | | | | | | WD-repeat cell cycle regulatory protein |
| 6790 | 2.514477448 | down | 0.000474442 | 0.002115917 | BM528763 | 557 | 98 | 145 | 67 | 1e−50 | |
| 6791 | 2.513711283 | down | 0.0000322 | 0.000312917 | AF452453.1 | | | | | | phosphate transporter |
| 6792 | 2.512908829 | down | 0.0000121 | 0.000159898 | CD406580 | 489 | 31 | 51 | 60 | 4e−13 | |
| 6793 | 2.512790023 | down | 0.0000117 | 0.000155589 | BI970498 | 794 | 113 | 137 | 82 | 3e−61 | galactose metabolism |
| 6794 | 2.512538141 | down | 0.00000813 | 0.00012179 | BI423924 | 559 | 58 | 99 | 58 | 4e−26 | aromatic amino acid family metabolism |
| 6795 | 2.512530635 | down | 0.0000653 | 0.000509425 | BE330995 | 570 | 50 | 91 | 54 | 8e−21 | |
| 6796 | 2.511640002 | down | 0.00000422 | 0.0000814 | BI471566 | 512 | 18 | 50 | 36 | 5e−04 | response to dessication |
| 6797 | 2.511554518 | down | 0.0000647 | 0.000506814 | BU544575 | 613 | 16 | 38 | 42 | 0.12 | proteolysis and peptidolysis |
| 6798 | 2.511374632 | down | 0.00000577 | 0.0000989 | CF808300 | 500 | 139 | 166 | 83 | 9e−81 | electron transport |
| 6799 | 2.511344946 | down | 0.0000203 | 0.000226572 | BI785492 | 549 | 143 | 183 | 78 | 3e−77 | |
| 6800 | 2.511194029 | down | 0.001560185 | 0.005019656 | BG041848 | 665 | 116 | 218 | 53 | 2e−56 | protein amino acid phosphorylation |
| 6801 | 2.511123228 | down | 0.000128384 | 0.000823648 | BI470370 | 574 | 113 | 191 | 59 | 5e−58 | biosynthesis |
| 6802 | 2.510620703 | down | 0.00120496 | 0.004162309 | AW133169 | 267 | 85 | 87 | 97 | 9e−43 | ubiquitin-dependent protein catabolism |
| 6803 | 2.510512765 | down | 0.0000309 | 0.0003044 | BF423878 | 385 | 20 | 67 | 29 | 1.8 | regulation of transcription |
| 6804 | 2.510433758 | down | 0.00000872 | 0.000127346 | BM732305 | 420 | 15 | 33 | 45 | 0.042 | DNA repair |
| 6805 | 2.51023951 | down | 0.00000652 | 0.000106503 | AW099842 | 415 | 26 | 52 | 50 | 2e−11 | carbohydrate metabolism |
| 6806 | 2.509065562 | down | 0.000108611 | 0.000729131 | AW185315 | 478 | 12 | 35 | 34 | 1.8 | protein amino acid phosphorylation |
| 6807 | 2.50894945 | down | 0.0000421 | 0.000376292 | BM892632 | 558 | 105 | 193 | 54 | 1e−55 | carbohydrate metabolism |
| 6808 | 2.508907429 | down | 0.0000482 | 0.000413128 | BE805991 | 392 | 35 | 135 | 25 | 0.001 | lipoprotein metabolism |
| 6809 | 2.508719155 | down | 0.001865602 | 0.005693068 | BG508620 | 494 | 122 | 163 | 74 | 6e−72 | regulation of transcription\, DNA- |
| | | | | | | | | | | | dependent |
| 6810 | 2.50842388 | down | 0.000561218 | 0.002398552 | CF806925 | 549 | 155 | 183 | 84 | 2e−87 | regulation of meristem organization |
| 6811 | 2.508395344 | down | 0.0000232 | 0.000249853 | AY258630.1 | | | | | | MRNA sequence |
| 6812 | 2.508266511 | down | 0.0000125 | 0.00016339 | BU926989 | 442 | 12 | 25 | 48 | 4.4 | |
| 6813 | 2.508251621 | down | 0.0000144 | 0.000180904 | BE657917 | 581 | 36 | 131 | 27 | 2e−10 | defense response |
| 6814 | 2.507981157 | down | 0.00000124 | 0.0000373 | BG510741 | 280 | 17 | 49 | 34 | 3.2 | metabolism |
| 6815 | 2.507456019 | down | 0.0000881 | 0.000629565 | BQ628787 | 575 | 106 | 193 | 54 | 2e−55 | response to oxidative stress |
| 6816 | 2.507000824 | down | 0.000256561 | 0.001353854 | BG839522 | 487 | 93 | 128 | 72 | 2e−50 | carbohydrate metabolism |
| 6817 | 2.506236749 | down | 0.0000185 | 0.000212553 | CD417806 | 641 | 49 | 87 | 56 | 2e−17 | |
| 6818 | 2.506030937 | down | 0.000037 | 0.000343325 | BI699931 | 524 | 23 | 94 | 24 | 0.003 | protein folding |
| 6819 | 2.505696522 | down | 0.000116307 | 0.000766561 | AW424283 | 445 | 22 | 30 | 73 | 8e−07 | |
| 6820 | 2.505534539 | down | 0.000375956 | 0.001784831 | BE058913 | 418 | 53 | 83 | 63 | 1e−24 | starch catabolism |
| 6821 | 2.50493216 | down | 0.000008 | 0.000120657 | CF808379 | 421 | 121 | 130 | 93 | 6e−63 | |
| 6822 | 2.504786037 | down | 0.00000554 | 0.0000966 | AW311022 | 600 | 28 | 58 | 48 | 7e−10 | electron transport |
| 6823 | 2.504704199 | down | 0.000111005 | 0.00074075 | BU551189 | 697 | 113 | 177 | 63 | 5e−61 | |
| 6824 | 2.504312336 | down | 0.0000385 | 0.000352804 | BG362680 | 563 | 36 | 52 | 69 | 5e−14 | |
| 6825 | 2.503974553 | down | 0.000288045 | 0.001473838 | BG551207 | 486 | 97 | 164 | 59 | 3e−50 | |
| 6826 | 2.503885995 | down | 0.000173114 | 0.001018743 | BQ740952 | 569 | 75 | 155 | 48 | 1e−31 | |
| 6827 | 2.503628742 | down | 0.00000291 | 0.0000644 | BI970635 | 791 | 110 | 130 | 84 | 3e−64 | electron transport |
| 6828 | 2.50362704 | down | 0.0000577 | 0.000468317 | CA784396 | 758 | 179 | 233 | 76 | e−109 | |
| 6829 | 2.503378487 | down | 0.0000166 | 0.000198056 | BU549566 | 602 | 70 | 139 | 50 | 2e−26 | |
| 6830 | 2.503284561 | down | 0.00043765 | 0.001993675 | BQ740268 | 599 | 96 | 140 | 68 | 1e−47 | regulation of transcription\, DNA- |
| | | | | | | | | | | | dependent |
| 6831 | 2.502779654 | down | 0.00183823 | 0.005635141 | BG790087 | 427 | 28 | 66 | 42 | 5e−08 | regulation of translation |
| 6832 | 2.502627905 | down | 0.0000305 | 0.000301412 | BU544212 | 451 | 38 | 47 | 80 | 1e−15 | |
| 6833 | 2.502561486 | down | 0.000189901 | 0.001085055 | BG725172 | 347 | 12 | 41 | 29 | 1.4 | oligopeptide transport |
| 6834 | 2.502199544 | down | 0.0000245 | 0.000258531 | BE023128 | 437 | 45 | 89 | 50 | 1e−14 | |
| 6835 | 2.502041055 | down | 0.00000879 | 0.000128192 | BE805348 | 355 | 46 | 110 | 41 | 2e−17 | |
| 6836 | 2.501967183 | down | 0.0000736 | 0.00055452 | CF921114 | 727 | 107 | 199 | 53 | 7e−61 | Glutathione S-transferase GST 13, |
| | | | | | | | | | | | transport |
| 6837 | 2.501193446 | down | 0.00000235 | 0.0000566 | AW351016 | 714 | 68 | 95 | 71 | 8e−38 | |
| 6838 | 2.500571816 | down | 0.0000166 | 0.000198394 | BM270865 | 458 | 107 | 153 | 69 | 9e−63 | carbohydrate metabolism |
| 6839 | 2.499911607 | down | 0.00000955 | 0.000135676 | BQ452943 | 579 | 125 | 195 | 64 | 4e−66 | lipid metabolism |
| 6840 | 2.499565467 | down | 0.000380929 | 0.001803657 | BI699942 | 305 | 9 | 26 | 34 | 0.50 | |
| 6841 | 2.499497275 | down | 0.00000356 | 0.0000731 | BU549924 | 529 | 49 | 60 | 81 | 3e−25 | |
| 6842 | 2.498934512 | down | 0.0000624 | 0.000494022 | BM309321 | 550 | 93 | 149 | 62 | 1e−50 | mitogen-activated protein kinase kinase |
| | | | | | | | | | | | MAPKK2, MAPKKK cascade |
| 6843 | 2.498916357 | down | 0.00000672 | 0.000108569 | CD400959 | 570 | 42 | 55 | 76 | 1e−17 | |
| 6844 | 2.498731295 | down | 0.0000291 | 0.000291143 | CA935989 | 456 | 11 | 27 | 40 | 2.1 | |
| 6845 | 2.497638032 | down | 0.0000209 | 0.000230735 | BI893077 | 555 | 99 | 118 | 83 | 4e−54 | cell differentiation |
| 6846 | 2.409819009 | down | 0.00000335 | 0.0000705 | AI441434 | 411 | 23 | 53 | 43 | 9e−07 | regulation of transcription\, DNA- |
| | | | | | | | | | | | dependent |
| 6847 | 2.40946425 | down | 0.00000549 | 0.0000961 | BQ612713 | 434 | 40 | 62 | 64 | 5e−15 | histidine biosynthesis |
| 6848 | 2.409008181 | down | 0.00000885 | 0.000128725 | BG511541 | 325 | 14 | 53 | 26 | 4.1 | |
| 6849 | 2.408900716 | down | 0.000068 | 0.000524508 | BI970399 | 392 | 16 | 52 | 30 | 5.7 | glycerol metabolism |
| 6850 | 2.407985344 | down | 0.0000314 | 0.000307673 | BE473433 | 414 | 34 | 84 | 40 | 2e−11 | |
| 6851 | 2.40757332 | down | 0.00000832 | 0.000123535 | CA801769 | 574 | 107 | 168 | 63 | 1e−53 | |
| 6852 | 2.407516826 | down | 0.0000114 | 0.000153042 | AW567738 | 563 | 101 | 194 | 52 | 3e−53 | |
| 6853 | 2.407353417 | down | 0.000463295 | 0.002078283 | BU761172 | 564 | 14 | 38 | 36 | 0.38 | photomorphogenesis |
| 6854 | 2.407209866 | down | 0.000047 | 0.000406543 | BE346170 | 605 | 111 | 183 | 80 | 5e−68 | xyloglucan biosynthesis |
| 6855 | 2.407041376 | down | 0.000277918 | 0.001433768 | AW424155 | | | | | | |
| 6856 | 2.40683786 | down | 0.00000291 | 0.0000644 | BE657958 | 569 | 23 | 41 | 56 | 5e−09 | |
| 6857 | 2.406226241 | down | 0.0000375 | 0.00034639 | BU081510 | 322 | 11 | 32 | 34 | 3.2 | regulation of transcription\, DNA- |
| | | | | | | | | | | | dependent |
| 6858 | 2.404940603 | down | 0.0000366 | 0.000341807 | BG652735 | 477 | 84 | 152 | 55 | 6e−44 | protein amino acid phosphorylation |
| 6859 | 2.404166235 | down | 0.00002 | 0.000223716 | AW306729 | 452 | 41 | 81 | 50 | 5e−17 | |
| 6860 | 2.403448912 | down | 0.0000721 | 0.000546444 | BI471792 | 525 | 62 | 88 | 70 | 3e−36 | |
| 6861 | 2.402632316 | down | 0.00000571 | 0.0000985 | AF502079.1 | | | | | | resistance protein KR3 |
| 6862 | 2.402095573 | down | 0.000306716 | 0.001542148 | AW351315 | 652 | 111 | 129 | 86 | 2e−58 | protein biosynthesis |
| 6863 | 2.402041765 | down | 0.0000747 | 0.000559647 | BI969847 | 780 | 112 | 177 | 63 | 3e−62 | carbohydrate metabolism |
| 6864 | 2.401809183 | down | 0.00000224 | 0.000055 | BQ630319 | 544 | 80 | 177 | 45 | 2e−35 | |
| 6865 | 2.496660009 | down | 0.0000168 | 0.000199563 | CD407677 | 498 | 46 | 59 | 77 | 4e−29 | |
| 6866 | 2.496446938 | down | 0.0000408 | 0.000367441 | CF808147 | 744 | 184 | 190 | 96 | e−100 | protein secretion |
| 6867 | 2.44700045 | down | 0.00000927 | 0.000132701 | BU548292 | 637 | 43 | 56 | 76 | 2e−19 | |
| 6868 | 2.446794589 | down | 0.000088 | 0.000629302 | BG362609 | 560 | 151 | 187 | 80 | 1e−83 | protein amino acid phosphorylation |
| 6869 | 2.445879342 | down | 0.000122701 | 0.000796974 | BE806991 | 344 | 16 | 45 | 35 | 0.29 | carbohydrate metabolism |
| 6870 | 2.445793486 | down | 0.0000716 | 0.000543777 | BI969902 | 794 | 93 | 119 | 78 | 9e−46 | |
| 6871 | 2.445349184 | down | 0.0000176 | 0.000205908 | BI969660 | 671 | 79 | 111 | 71 | 3e−41 | methionine metabolism |
| 6872 | 2.445075284 | down | 0.000170645 | 0.001007481 | CF806338 | 641 | 16 | 47 | 34 | 0.21 | transport |
| 6873 | 2.444801293 | down | 0.0000215 | 0.00023567 | BE473574 | 536 | 92 | 137 | 67 | 1e−53 | cytolysis |
| 6874 | 2.444518037 | down | 0.00000468 | 0.000087 | BE022652 | 147 | 17 | 22 | 77 | 4e−05 | |
| 6875 | 2.444427818 | down | 0.0000509 | 0.000428124 | BU551398 | 568 | 129 | 139 | 92 | 2e−71 | cellulose biosynthesis |
| 6876 | 2.444388053 | down | 0.0000261 | 0.000270659 | CD402148 | 606 | 59 | 75 | 78 | 1e−32 | |
| 6877 | 2.444291396 | down | 0.0000935 | 0.000657032 | AW831824 | 381 | 81 | 125 | 64 | 8e−38 | |
| 6878 | 2.444046709 | down | 0.00000782 | 0.000119065 | CD398750 | 542 | 55 | 71 | 77 | 3e−23 | protein amino acid phosphorylation |
| 6879 | 2.443757181 | down | 0.000213504 | 0.001183425 | BM893352 | 535 | 60 | 164 | 36 | 2e−13 | |
| 6880 | 2.443740664 | down | 0.0000675 | 0.000521698 | BF425767 | 583 | 133 | 194 | 68 | 3e−79 | |
| 6881 | 2.442236579 | down | 0.0000207 | 0.0002292 | AW733781 | 600 | 126 | 199 | 63 | 1e−71 | carbohydrate metabolism |
| 6882 | 2.441738538 | down | 0.0000255 | 0.000266196 | CK605849 | 606 | 62 | 73 | 84 | 1e−32 | |
| 6883 | 2.441560014 | down | 0.0000255 | 0.000266386 | BG156924 | 395 | 43 | 92 | 46 | 2e−17 | |
| 6884 | 2.441541394 | down | 0.00000348 | 0.0000723 | AW164497 | 353 | 23 | 63 | 36 | 2e−04 | |
| 6885 | 2.441258898 | down | 0.00000788 | 0.000119427 | CD395873 | 622 | 32 | 115 | 27 | 1e−04 | regulation of transcription\, DNA- |
| | | | | | | | | | | | dependent |
| 6886 | 2.440892573 | down | 0.0000578 | 0.000468361 | CD400770 | 549 | 36 | 71 | 50 | 8e−12 | |
| 6887 | 2.439902839 | down | 0.00000419 | 0.000081 | BQ298856 | 419 | 54 | 64 | 84 | 1e−27 | galactose metabolism |
| 6888 | 2.438873672 | down | 0.00000589 | 0.00010008 | AF271071.1 | | | | | | biotin carboxyl carrier protein subunit |
| | | | | | | | | | | | precursor |
| 6889 | 2.4386554 | down | 0.00011905 | 0.000779721 | AW308995 | 502 | 76 | 169 | 44 | 2e−35 | electron transport |
| 6890 | 2.438484383 | down | 0.0000146 | 0.000182232 | BU547946 | 676 | 102 | 162 | 62 | 7e−67 | |
| 6891 | 2.496215454 | down | 0.00000673 | 0.000108581 | CD393345 | 426 | 30 | 59 | 50 | 9e−11 | |
| 6892 | 2.496113226 | down | 0.001231444 | 0.004232027 | BG042249 | 339 | 54 | 69 | 78 | 1e−30 | |
| 6893 | 2.495991442 | down | 0.0000395 | 0.000358965 | BM522940 | 424 | 12 | 14 | 85 | 0.21 | |
| 6894 | 2.495221064 | down | 0.00000176 | 0.0000468 | AY028297.1 | | | | | | lipoxygenase |
| 6895 | 2.493602299 | down | 0.0000358 | 0.000336872 | BE819945 | 598 | 50 | 64 | 78 | 5e−23 | protein-nucleus import |
| 6896 | 2.493354444 | down | 0.00047664 | 0.002124463 | BQ611306 | 356 | 14 | 29 | 48 | 2.5 | |
| 6897 | 2.493165105 | down | 0.0000851 | 0.000614319 | AI496624 | 408 | 39 | 91 | 42 | 9e−18 | 2-hydroxyisoflavanone dehydratase |
| 6898 | 2.49304493 | down | 0.0000385 | 0.000352804 | CF808771 | 565 | 56 | 161 | 34 | 7e−17 | |
| 6899 | 2.492766749 | down | 0.0000542 | 0.000447122 | BQ628857 | 253 | 18 | 60 | 30 | 7.3 | electron transport |
| 6900 | 2.492344762 | down | 0.0000127 | 0.000165597 | CA936677 | 479 | 60 | 64 | 93 | 2e−34 | carbohydrate biosynthesis |
| 6901 | 2.492026942 | down | 0.0000163 | 0.000196399 | BG237258 | 393 | 11 | 35 | 31 | 1.5 | cytoskeleton organization and |
| | | | | | | | | | | | biogenesis |
| 6902 | 2.4917632 | down | 0.000267113 | 0.001393236 | BM309583 | 576 | 22 | 23 | 95 | 4e−06 | regulation of transcription\, DNA- |
| | | | | | | | | | | | dependent |
| 6903 | 2.491728321 | down | 0.000105605 | 0.000714175 | CF808607 | 558 | 83 | 161 | 51 | 2e−36 | protein amino acid phosphorylation |
| 6904 | 2.491668942 | down | 0.00000585 | 0.0000999 | AW598510 | 439 | 56 | 93 | 60 | 2e−28 | regulation of transcription |
| 6905 | 2.491105552 | down | 0.00000581 | 0.0000994 | AW350843 | 597 | 45 | 76 | 59 | 9e−23 | |
| 6906 | 2.490938348 | down | 0.0000406 | 0.000366408 | BG508657 | 481 | 31 | 111 | 27 | 0.48 | protein amino acid phosphorylation |
| 6907 | 2.490840528 | down | 0.0000202 | 0.000224994 | BF070096 | 475 | 85 | 105 | 80 | 9e−48 | |
| 6908 | 2.490476288 | down | 0.0000444 | 0.00039034 | BF069997 | 498 | 93 | 166 | 56 | 1e−44 | protein amino acid phosphorylation |
| 6909 | 2.490459991 | down | 0.00000253 | 0.0000594 | BE610331 | 233 | 9 | 23 | 39 | 1.9 | |
| 6910 | 2.490329814 | down | 0.000555221 | 0.002378565 | AW781592 | 276 | 14 | 44 | 31 | 1.9 | |
| 6911 | 2.489931149 | down | 0.000962281 | 0.003554477 | CD416307 | 694 | 14 | 28 | 50 | 0.11 | |
| 6912 | 2.488710549 | down | 0.000072 | 0.000545517 | AW102065 | 626 | 50 | 178 | 28 | 3e−06 | |
| 6913 | 2.488657249 | down | 0.00000412 | 0.0000802 | CD418598 | 606 | 52 | 110 | 47 | 4e−19 | |
| 6914 | 2.488368335 | down | 0.000184989 | 0.001064432 | AW756277 | | | | | | |
| 6915 | 2.488244485 | down | 0.000341171 | 0.001661636 | AI736243 | 235 | 11 | 29 | 37 | 7.1 | trehalose biosynthesis |
| 6916 | 2.48755432 | down | 0.000387185 | 0.001824821 | BI786323 | 560 | 80 | 110 | 72 | 4e−46 | |
| 6917 | 2.485873911 | down | 0.0000472 | 0.000407632 | AI495850 | 519 | 69 | 145 | 47 | 1e−31 | |
| 6918 | 2.48564618 | down | 0.0000201 | 0.000224309 | BQ630441 | 612 | 45 | 62 | 72 | 2e−24 | |
| 6919 | 2.485311348 | down | 0.000215734 | 0.001192626 | BE659136 | 758 | 25 | 88 | 28 | 0.001 | |
| 6920 | 2.484942685 | down | 0.00000894 | 0.000129628 | BI700179 | 312 | 45 | 53 | 84 | 1e−20 | DNA metabolism |
| 6921 | 2.484872849 | down | 0.000323197 | 0.001600411 | BE475334 | 430 | 125 | 142 | 88 | 3e−71 | |
| 6922 | 2.484358513 | down | 0.0000459 | 0.000399746 | CA936923 | 547 | 112 | 182 | 61 | 1e−57 | protein amino acid phosphorylation |
| 6923 | 2.484066407 | down | 0.0000252 | 0.000264717 | BG363381 | 481 | 40 | 115 | 34 | 6e−10 | response to red/far-red light |
| 6924 | 2.483552071 | down | 0.00000207 | 0.0000521 | CD412043 | 425 | 11 | 25 | 44 | 0.019 | |
| 6925 | 2.483516884 | down | 0.000105021 | 0.000711375 | BI426517 | 570 | 101 | 141 | 71 | 6e−56 | transport |
| 6926 | 2.483348803 | down | 0.00000132 | 0.0000388 | CA938233 | 560 | 20 | 29 | 68 | 7e−06 | |
| 6927 | 2.482756609 | down | 0.0000757 | 0.000565194 | AW279193 | 614 | 104 | 202 | 51 | 4e−50 | |
| 6928 | 2.482220339 | down | 0.000139184 | 0.000870905 | BE610010 | | | | | | |
| 6929 | 2.482200947 | down | 0.00000533 | 0.0000944 | BG237839 | 330 | 85 | 103 | 82 | 3e−45 | protein amino acid phosphorylation |
| 6930 | 2.480872095 | down | 0.0000172 | 0.000202511 | BQ252680 | 462 | 61 | 75 | 81 | 8e−32 | |
| 6931 | 2.480716756 | down | 0.00000681 | 0.000109353 | CA784454 | 629 | 51 | 131 | 38 | 6e−12 | proton transport |
| 6932 | 2.480595332 | down | 0.000315618 | 0.001575861 | AW133007 | 515 | 103 | 156 | 66 | 4e−48 | regulation of transcription\, DNA- |
| | | | | | | | | | | | dependent |
| 6933 | 2.480308272 | down | 0.0000554 | 0.000454382 | BU578999 | 434 | 115 | 134 | 85 | 3e−62 | malate dehydrogenase, tricarboxylic |
| | | | | | | | | | | | acid cycle |
| 6934 | 2.480293178 | down | 0.000922686 | 0.003446125 | BQ628223 | 610 | 160 | 204 | 78 | 7e−89 | ion transport |
| 6935 | 2.480127988 | down | 0.000013 | 0.000168102 | CD415189 | 656 | 91 | 104 | 87 | 4e−48 | protein biosynthesis |
| 6936 | 2.478873444 | down | 0.000472696 | 0.002109634 | BQ611378 | 593 | 84 | 157 | 53 | 1e−39 | |
| 6937 | 2.478806322 | down | 0.00000593 | 0.000100484 | CD398315 | 269 | 15 | 23 | 65 | 0.65 | |
| 6938 | 2.478511009 | down | 0.00000851 | 0.00012527 | BE803555 | 372 | 20 | 23 | 86 | 5e−06 | electron transport |
| 6939 | 2.477586353 | down | 0.00000688 | 0.000109986 | CD406410 | 604 | 29 | 85 | 34 | 0.067 | flower development |
| 6940 | 2.477251167 | down | 0.0000145 | 0.000181002 | AW706795 | 551 | 68 | 99 | 68 | 7e−32 | |
| 6941 | 2.477204993 | down | 0.00015089 | 0.000920615 | BE804124 | 384 | 35 | 77 | 45 | 2e−11 | response to light |
| 6942 | 2.477086105 | down | 0.00000551 | 0.0000963 | BU544185 | 555 | 55 | 71 | 77 | 1e−30 | electron transport |
| 6943 | 2.476397463 | down | 0.0000745 | 0.000559234 | BE824150 | 720 | 97 | 137 | 70 | 9e−52 | |
| 6944 | 2.476309417 | down | 0.0000349 | 0.000330817 | BF219559 | 691 | 88 | 102 | 86 | 5e−45 | L-serine biosynthesis |
| 6945 | 2.476029331 | down | 0.000107599 | 0.000724144 | BQ473444 | 574 | 114 | 165 | 69 | 7e−64 | |
| 6946 | 2.47557351 | down | 0.0000197 | 0.000221672 | CD408566 | 435 | 14 | 50 | 28 | 0.86 | |
| 6947 | 2.475005191 | down | 0.0000032 | 0.0000684 | CD410308 | 657 | 92 | 171 | 53 | 2e−41 | |
| 6948 | 2.474997001 | down | 0.0000146 | 0.000181775 | AW310275 | 497 | 19 | 58 | 32 | 1.2 | |
| 6949 | 2.474624306 | down | 0.0000374 | 0.000345834 | BI970248 | 646 | 70 | 104 | 67 | 7e−37 | |
| 6950 | 2.474552266 | down | 0.0000486 | 0.00041453 | BQ079684 | 559 | 61 | 176 | 34 | 2e−24 | protein amino acid phosphorylation |
| 6951 | 2.474439192 | down | 0.00000461 | 0.000086 | BQ297975 | 482 | 60 | 80 | 75 | 1e−33 | dicarboxylic acid transport |
| 6952 | 2.474348291 | down | 0.000177286 | 0.001034053 | CD407905 | 607 | 70 | 87 | 80 | 8e−37 | protein biosynthesis |
| 6953 | 2.474011782 | down | 0.00000708 | 0.000112018 | BM732317 | 565 | 62 | 106 | 58 | 2e−31 | |
| 6954 | 2.473368175 | down | 0.0000173 | 0.000203579 | CD412380 | 527 | 37 | 107 | 34 | 2e−08 | metal ion transport |
| 6955 | 2.473364548 | down | 0.0000271 | 0.000277457 | BU545886 | 573 | 30 | 40 | 75 | 7e−09 | regulation of transcription\, DNA- |
| | | | | | | | | | | | dependent |
| 6956 | 2.472187631 | down | 0.0000179 | 0.000208124 | CA783510 | 705 | 36 | 61 | 59 | 7e−15 | |
| 6957 | 2.47210614 | down | 0.000120553 | 0.000787372 | CF920420 | 629 | 82 | 156 | 52 | 7e−42 | |
| 6958 | 2.472006435 | down | 0.00000202 | 0.0000512 | CF806184 | 476 | 88 | 136 | 64 | 1e−43 | glucose catabolism |
| 6959 | 2.471635647 | down | 0.000172292 | 0.00101481 | BE659804 | 529 | 86 | 111 | 77 | 7e−42 | transport |
| 6960 | 2.471551196 | down | 0.000416512 | 0.001924537 | BE474062 | 410 | 46 | 61 | 75 | 5e−21 | |
| 6961 | 2.471444339 | down | 0.0000391 | 0.000356022 | AW349604 | 424 | 47 | 58 | 81 | 6e−23 | |
| 6962 | 2.47140587 | down | 0.000012 | 0.000158814 | AW309969 | 559 | 51 | 63 | 80 | 3e−23 | |
| 6963 | 2.471165187 | down | 0.000011 | 0.000150342 | BU547906 | 620 | 11 | 31 | 35 | 3.9 | |
| 6964 | 2.470301238 | down | 0.00000147 | 0.0000417 | BU546918 | 367 | 35 | 53 | 66 | 6e−17 | amino acid metabolism |
| 6965 | 2.46938515 | down | 0.00000509 | 0.0000921 | BE190802 | 444 | 17 | 28 | 60 | 5e−06 | |
| 6966 | 2.468834274 | down | 0.000051 | 0.000428538 | AI899921 | 663 | 126 | 216 | 58 | 2e−67 | |
| 6967 | 2.46838403 | down | 0.00000766 | 0.000117558 | AW396739 | 658 | 135 | 200 | 67 | 1e−68 | |
| 6968 | 2.466968564 | down | 0.0000081 | 0.000121459 | BQ740576 | 590 | 90 | 165 | 54 | 4e−47 | protein amino acid phosphorylation |
| 6969 | 2.466442354 | down | 0.000188172 | 0.001078003 | BI469916 | 324 | 12 | 25 | 48 | 4.1 | |
| 6970 | 2.466123826 | down | 0.00000634 | 0.000105028 | BM526502 | 565 | 54 | 184 | 29 | 4e−12 | |
| 6971 | 2.46556161 | down | 0.00000853 | 0.000125465 | BQ785508 | 518 | 62 | 91 | 68 | 2e−31 | ubiquitin-dependent protein catabolism |
| 6972 | 2.465553601 | down | 0.0000191 | 0.000217575 | BG047084 | 447 | 23 | 51 | 45 | 4e−04 | |
| 6973 | 2.464981107 | down | 0.00000814 | 0.000121827 | BE822226 | 654 | 131 | 187 | 70 | 1e−73 | |
| 6974 | 2.464156025 | down | 0.00000817 | 0.000122179 | AW234386 | 583 | 88 | 142 | 61 | 2e−46 | ion transport |
| 6975 | 2.464012906 | down | 0.00000263 | 0.0000608 | CD405909 | 516 | 16 | 48 | 33 | 0.42 | RNA dependent DNA replication |
| 6976 | 2.462032685 | down | 0.000139421 | 0.000871644 | BI969450 | 767 | 108 | 183 | 59 | 8e−57 | |
| 6977 | 2.461422098 | down | 0.0000776 | 0.000574307 | BF595470 | 448 | 19 | 74 | 25 | 1.2 | |
| 6978 | 2.461346839 | down | 0.0000159 | 0.000192924 | BQ253856 | 482 | 102 | 162 | 62 | 1e−46 | regulation of transcription |
| 6979 | 2.461180129 | down | 0.0000176 | 0.000206275 | BM891305 | 467 | 38 | 81 | 46 | 3e−14 | |
| 6980 | 2.461083754 | down | 0.0000138 | 0.000174959 | BI427009 | 571 | 131 | 165 | 79 | 2e−69 | transport |
| 6981 | 2.461061403 | down | 0.0000644 | 0.000505192 | CD405906 | 438 | 24 | 79 | 30 | 0.52 | |
| 6982 | 2.460966848 | down | 0.0000721 | 0.000546401 | CA802562 | 734 | 60 | 70 | 85 | 2e−30 | regulation of transcription |
| 6983 | 2.460315873 | down | 0.00000869 | 0.000127133 | BU550176 | 562 | 15 | 49 | 30 | 1.1 | regulation of transcription |
| 6984 | 2.459553694 | down | 0.0000112 | 0.000151895 | BE658608 | 363 | 12 | 29 | 41 | 3.5 | |
| 6985 | 2.459311297 | down | 0.000047 | 0.000406543 | AW598033 | 273 | 21 | 75 | 28 | 2.4 | proline biosynthesis |
| 6986 | 2.458660001 | down | 0.0000114 | 0.000153515 | BU545776 | | | | | | |
| 6987 | 2.457708921 | down | 0.001887731 | 0.005746386 | AW202391 | 806 | 69 | 120 | 57 | 2e−29 | |
| 6988 | 2.457634724 | down | 0.0000493 | 0.000418849 | BQ473675 | 456 | 71 | 80 | 88 | 6e−38 | transport |
| 6989 | 2.456943653 | down | 0.000925791 | 0.003454634 | BG881477 | 493 | 118 | 168 | 70 | 1e−60 | |
| 6990 | 2.45687719 | down | 0.000117238 | 0.000770675 | BQ453969 | 382 | 25 | 99 | 25 | 3.1 | regulation of transcription\, DNA- |
| | | | | | | | | | | | dependent |
| 6991 | 2.45561519 | down | 0.00000358 | 0.0000732 | CD393758 | 392 | 29 | 30 | 96 | 7e−09 | proton transport |
| 6992 | 2.455528236 | down | 0.0000188 | 0.000215218 | BI470614 | 422 | 58 | 92 | 63 | 5e−35 | |
| 6993 | 2.455506865 | down | 0.00000727 | 0.000114049 | BG044327 | 483 | 61 | 71 | 85 | 9e−29 | protein amino acid phosphorylation |
| 6994 | 2.454797492 | down | 0.000250392 | 0.001328947 | AW756993 | 419 | 47 | 83 | 56 | 3e−22 | protein amino acid phosphorylation |
| 6995 | 2.453926344 | down | 0.00000521 | 0.0000932 | BQ612664 | 505 | 74 | 97 | 76 | 2e−41 | |
| 6996 | 2.453384135 | down | 0.00000975 | 0.00013777 | CD413589 | 327 | 63 | 75 | 84 | 5e−36 | |
| 6997 | 2.452942526 | down | 0.00000568 | 0.0000982 | CD392019 | 597 | 14 | 44 | 31 | 8.0 | |
| 6998 | 2.452839413 | down | 0.00000549 | 0.0000961 | BE347570 | 484 | 71 | 240 | 29 | 3e−10 | regulation of transcription |
| 6999 | 2.45238503 | down | 0.0000139 | 0.00017579 | BI972054 | 532 | 57 | 119 | 47 | 1e−24 | proline dehydrogenase, glutamate |
| | | | | | | | | | | | biosynthesis |
| 7000 | 2.451614832 | down | 0.0000985 | 0.000680833 | BU765584 | 420 | 31 | 35 | 88 | 5e−11 | |
| 7001 | 2.451457489 | down | 0.0000106 | 0.000145825 | AI966188 | 523 | 93 | 188 | 49 | 9e−42 | |
| 7002 | 2.451301744 | down | 0.00000379 | 0.0000761 | AW100632 | 253 | 26 | 85 | 30 | 0.17 | |
| 7003 | 2.450944388 | down | 0.0000191 | 0.000217074 | CD408574 | 635 | 74 | 158 | 46 | 1e−34 | protein amino acid phosphorylation |
| 7004 | 2.450778952 | down | 0.00000461 | 0.000086 | BI424687 | 586 | 111 | 194 | 57 | 2e−63 | galactose metabolism |
| 7005 | 2.450655958 | down | 0.00000514 | 0.0000927 | BG839320 | 826 | 122 | 167 | 73 | 2e−68 | protein amino acid phosphorylation |
| 7006 | 2.450081395 | down | 0.0000271 | 0.000277328 | BG352358 | 470 | 61 | 107 | 57 | 3e−32 | |
| 7007 | 2.449310032 | down | 0.0000361 | 0.000338456 | CA935091 | 608 | 72 | 200 | 36 | 3e−22 | |
| 7008 | 2.449095087 | down | 0.0000265 | 0.000273713 | BG510325 | 495 | 73 | 167 | 43 | 8e−31 | protein amino acid phosphorylation |
| 7009 | 2.448931409 | down | 0.000187757 | 0.00107685 | CD395597 | 524 | 54 | 102 | 52 | 7e−23 | |
| 7010 | 2.448760349 | down | 0.00000643 | 0.000105803 | BG239730 | 460 | 46 | 60 | 76 | 2e−21 | regulation of transcription |
| 7011 | 2.448272508 | down | 0.0000654 | 0.000509812 | BG509010 | 397 | 22 | 68 | 32 | 5e−04 | defense response |
| 7012 | 2.447958227 | down | 0.000160451 | 0.000964041 | BE822184 | 635 | 92 | 137 | 67 | 2e−52 | cell wall modification |
| 7013 | 2.447787861 | down | 0.0000218 | 0.000237664 | CF805902 | 672 | 94 | 222 | 42 | 8e−45 | metabolism |
| 7014 | 2.447341771 | down | 0.0000276 | 0.000281287 | BQ298051 | 550 | 119 | 127 | 93 | 2e−66 | protein amino acid phosphorylation |
| 7015 | 2.447162291 | down | 0.000102312 | 0.000698812 | CD416202 | 661 | 52 | 93 | 55 | 7e−24 | |
| 7016 | 2.437546534 | down | 0.0000124 | 0.000162784 | BE820594 | 671 | 42 | 69 | 60 | 1e−17 | metal ion transport |
| 7017 | 2.436456329 | down | 0.0000803 | 0.000589277 | BF597599 | 418 | 31 | 122 | 25 | 8e−04 | defense response |
| 7018 | 2.43633674 | down | 0.00022715 | 0.001236884 | CD417799 | 685 | 72 | 94 | 76 | 8e−37 | |
| 7019 | 2.436139981 | down | 0.0000204 | 0.000226699 | BE330811 | 395 | 83 | 130 | 63 | 4e−43 | |
| 7020 | 2.4356855 | down | 0.00000182 | 0.0000478 | BG509756 | 504 | 113 | 152 | 74 | 1e−65 | |
| 7021 | 2.43550113 | down | 0.00000544 | 0.0000955 | BE022807 | | | | | | |
| 7022 | 2.435313899 | down | 0.000169168 | 0.001000802 | CA800458 | 789 | 235 | 264 | 89 | e−136 | |
| 7023 | 2.434857048 | down | 0.000500192 | 0.002198695 | BI468894 | 516 | 29 | 57 | 50 | 5e−07 | regulation of transcription\, DNA- |
| | | | | | | | | | | | dependent |
| 7024 | 2.434619345 | down | 0.000979081 | 0.003601167 | CD393936 | 385 | 20 | 77 | 25 | 2.4 | |
| 7025 | 2.433882179 | down | 0.000203417 | 0.001141623 | AW733698 | 437 | 35 | 47 | 74 | 8e−15 | |
| 7026 | 2.43382638 | down | 0.0000157 | 0.00019101 | AW102372 | 414 | 38 | 136 | 27 | 4e−04 | |
| 7027 | 2.432305816 | down | 0.0000237 | 0.000252878 | CK605907 | 754 | 82 | 145 | 56 | 1e−37 | |
| 7028 | 2.432252368 | down | 0.0000304 | 0.000301074 | CD417318 | 365 | 9 | 36 | 25 | 1.6 | |
| 7029 | 2.432202131 | down | 0.000670744 | 0.0027222 | BE440603 | 631 | 123 | 186 | 66 | 6e−76 | cell growth and/or maintenance |
| 7030 | 2.431757807 | down | 0.0000342 | 0.000326647 | AW348263 | 613 | 74 | 94 | 78 | 2e−38 | |
| 7031 | 2.430614476 | down | 0.00000604 | 0.000101853 | BF068523 | 389 | 102 | 129 | 79 | 1e−59 | carbohydrate metabolism |
| 7032 | 2.430451281 | down | 0.0000582 | 0.000470306 | CF809155 | 647 | 113 | 198 | 57 | 2e−62 | purple acid phosphatase |
| 7033 | 2.430333602 | down | 0.0000512 | 0.000429728 | AF160197.1 | | | | | | Ni-binding urease accessory protein |
| | | | | | | | | | | | UreG |
| 7034 | 2.430077492 | down | 0.000013 | 0.00016783 | BI969887 | 798 | 121 | 183 | 66 | 1e−58 | protein amino acid phosphorylation |
| 7035 | 2.429598428 | down | 0.0000601 | 0.000480987 | BI787403 | 548 | 103 | 183 | 56 | 9e−51 | transport |
| 7036 | 2.429576244 | down | 0.00028083 | 0.001445618 | BU083376 | 427 | 78 | 140 | 55 | 4e−37 | |
| 7037 | 2.429558146 | down | 0.0000206 | 0.000228516 | BU550882 | 452 | 70 | 109 | 64 | 1e−36 | |
| 7038 | 2.429360504 | down | 0.00000415 | 0.0000806 | BG238044 | 515 | 49 | 105 | 46 | 5e−11 | |
| 7039 | 2.428954234 | down | 0.00032527 | 0.001608138 | CD396280 | 659 | 35 | 59 | 59 | 3e−13 | |
| 7040 | 2.36755095 | down | 0.0000084 | 0.00012419 | AB040040.1 | | | | | | nonclathrin coat protein zeta 1-COP |
| 7041 | 2.366506088 | down | 0.0000092 | 0.000132236 | BE347874 | 619 | 89 | 155 | 57 | 4e−44 | |
| 7042 | 2.366471271 | down | 0.0000152 | 0.000188139 | BE802496 | 553 | 84 | 188 | 44 | 6e−33 | protein amino acid phosphorylation |
| 7043 | 2.366211306 | down | 0.0000707 | 0.000539463 | BI701318 | 421 | 15 | 34 | 44 | 1.0 | |
| 7044 | 2.366058942 | down | 0.00000335 | 0.0000705 | CA782430 | 536 | 56 | 107 | 52 | 2e−28 | |
| 7045 | 2.366039768 | down | 0.0000359 | 0.000337272 | BG047414 | 439 | 10 | 18 | 55 | 0.52 | |
| 7046 | 2.366036641 | down | 0.00000567 | 0.000098 | AF363021.1 | | | | | | cytosolic glutamine synthetase beta 2 |
| 7047 | 2.366007074 | down | 0.00000323 | 0.0000688 | BG316320 | 400 | 76 | 105 | 72 | 1e−41 | |
| 7048 | 2.365794492 | down | 0.0000278 | 0.000281906 | BG352137 | 427 | 56 | 58 | 96 | 2e−28 | |
| 7049 | 2.365680004 | down | 0.000036 | 0.000337687 | BE821647 | 560 | 98 | 143 | 68 | 2e−51 | aromatic amino acid family |
| | | | | | | | | | | | biosynthesis\, shikimate pathway |
| 7050 | 2.365567995 | down | 0.00000817 | 0.000122212 | BU547912 | 633 | 74 | 170 | 43 | 4e−34 | |
| 7051 | 2.365460672 | down | 0.0000077 | 0.000117791 | BE609300 | 431 | 20 | 40 | 50 | 9e−06 | |
| 7052 | 2.365257018 | down | 0.00000535 | 0.0000946 | AW309947 | 499 | 36 | 50 | 72 | 2e−16 | |
| 7053 | 2.364992113 | down | 0.0000559 | 0.000457664 | BG237643 | 319 | 13 | 26 | 50 | 0.64 | |
| 7054 | 2.364971797 | down | 0.00000727 | 0.000114049 | BF070937 | 516 | 61 | 69 | 88 | 1e−28 | |
| 7055 | 2.364927629 | down | 0.00000372 | 0.0000753 | CD393324 | 524 | 65 | 78 | 83 | 1e−34 | fatty acid alpha-oxidation |
| 7056 | 2.364918394 | down | 0.0000248 | 0.000261224 | BG882742 | 507 | 9 | 20 | 45 | 7.7 | transport |
| 7057 | 2.364772676 | down | 0.00000994 | 0.000139724 | BU089706 | 383 | 92 | 126 | 73 | 1e−48 | glycerol metabolism |
| 7058 | 2.364646726 | down | 0.000399175 | 0.00186481 | BE329894 | 525 | 63 | 94 | 67 | 9e−31 | |
| 7059 | 2.364418175 | down | 0.001249276 | 0.00427262 | BE802488 | 591 | 117 | 197 | 59 | 2e−57 | starch metabolism |
| 7060 | 2.364307742 | down | 0.0000066 | 0.000107204 | BQ785555 | 276 | 17 | 35 | 48 | 4.2 | protein amino acid phosphorylation |
| 7061 | 2.364163528 | down | 0.000155653 | 0.00094249 | AI495341 | 451 | 44 | 77 | 57 | 5e−17 | |
| 7062 | 2.363330385 | down | 0.0000213 | 0.000234181 | AI441710 | 416 | 40 | 45 | 88 | 5e−16 | |
| 7063 | 2.362843842 | down | 0.000436636 | 0.001990728 | AW396525 | 656 | 196 | 218 | 89 | e−117 | |
| 7064 | 2.362700644 | down | 0.000110419 | 0.000738063 | BU544761 | 449 | 12 | 20 | 60 | 2.1 | |
| 7065 | 2.362672525 | down | 0.000224043 | 0.001224701 | BI971366 | 610 | 29 | 67 | 43 | 2e−07 | |
| 7066 | 2.362603536 | down | 0.0000732 | 0.000552236 | BM107945 | 525 | 120 | 151 | 79 | 8e−67 | pS257 protein |
| 7067 | 2.362394124 | down | 0.0000156 | 0.000190931 | CD413732 | 615 | 18 | 20 | 90 | 1e−04 | photosynthesis light harvesting |
| 7068 | 2.362357677 | down | 0.0000737 | 0.000555028 | CD415625 | 655 | 112 | 151 | 74 | 1e−64 | |
| 7069 | 2.36226281 | down | 0.000192289 | 0.001094875 | AB052786.1 | | | | | | NRT1-3 protein |
| 7070 | 2.362090496 | down | 0.0000276 | 0.000280947 | BM567736 | 521 | 37 | 90 | 41 | 1e−12 | apoptosis |
| 7071 | 2.361923356 | down | 0.0000159 | 0.000193231 | CA936646 | 608 | 160 | 206 | 77 | 9e−92 | |
| 7072 | 2.360787956 | down | 0.000221885 | 0.001216615 | BG239631 | 379 | 65 | 81 | 80 | 2e−31 | pentose-phosphate shunt |
| 7073 | 2.360469655 | down | 0.00000704 | 0.000111453 | BQ273518 | 385 | 10 | 25 | 40 | 9.1 | carbohydrate metabolism |
| 7074 | 2.360276993 | down | 0.00000797 | 0.000120381 | BI426941 | 512 | 42 | 61 | 68 | 7e−22 | |
| 7075 | 2.428804612 | down | 0.0000911 | 0.000646051 | BQ452478 | 317 | 25 | 32 | 78 | 1e−08 | |
| 7076 | 2.428745046 | down | 0.0000119 | 0.000157678 | AW306744 | | | | | | |
| 7077 | 2.428735998 | down | 0.0000359 | 0.000337147 | BU548027 | 569 | 108 | 152 | 71 | 2e−57 | proteolysis and peptidolysis |
| 7078 | 2.428721538 | down | 0.0000947 | 0.000662806 | BI471571 | 364 | 29 | 87 | 33 | 0.037 | |
| 7079 | 2.428308136 | down | 0.00000829 | 0.000123258 | BQ612173 | 515 | 55 | 101 | 54 | 2e−23 | nucleosome assembly |
| 7080 | 2.428037171 | down | 0.00000555 | 0.0000967 | BU763690 | 274 | 44 | 91 | 48 | 7e−19 | metabolism |
| 7081 | 2.426942372 | down | 0.0000728 | 0.000549748 | BG047068 | 423 | 34 | 56 | 60 | 6e−15 | carbohydrate metabolism |
| 7082 | 2.426756608 | down | 0.0000294 | 0.000293021 | CA801072 | 408 | 24 | 52 | 46 | 1e−09 | |
| 7083 | 2.426206353 | down | 0.00014491 | 0.00089472 | CA934979 | 605 | 132 | 201 | 65 | 1e−76 | electron transport |
| 7084 | 2.426007602 | down | 0.0000238 | 0.000253843 | BG043044 | 578 | 138 | 196 | 70 | 2e−75 | protein amino acid phosphorylation |
| 7085 | 2.425677761 | down | 0.0000649 | 0.000507941 | CD396318 | 671 | 63 | 87 | 72 | 2e−33 | |
| 7086 | 2.425661897 | down | 0.0000226 | 0.000244407 | BI427326 | 545 | 93 | 96 | 96 | 7e−50 | small GTPase mediated signal |
| | | | | | | | | | | | transduction |
| 7087 | 2.425369268 | down | 0.00000515 | 0.0000928 | AI973637 | 482 | 84 | 136 | 61 | 4e−40 | |
| 7088 | 2.425307015 | down | 0.0000308 | 0.000303081 | BG881710 | 404 | 45 | 88 | 51 | 7e−17 | |
| 7089 | 2.425036595 | down | 0.00000358 | 0.0000732 | AW570024 | 316 | 53 | 73 | 72 | 9e−27 | |
| 7090 | 2.424827013 | down | 0.00000294 | 0.000065 | BE020729 | 484 | 12 | 37 | 32 | 1.8 | |
| 7091 | 2.424332783 | down | 0.00000845 | 0.000124606 | BG551593 | 391 | 47 | 81 | 58 | 1e−19 | |
| 7092 | 2.424292892 | down | 0.00000242 | 0.0000577 | BE824264 | 732 | 113 | 175 | 64 | 9e−63 | carbohydrate metabolism |
| 7093 | 2.424264262 | down | 0.0000523 | 0.000436138 | BE806733 | 441 | 27 | 96 | 28 | 0.010 | |
| 7094 | 2.424203965 | down | 0.00000752 | 0.00011624 | BI971682 | 568 | 94 | 189 | 49 | 4e−45 | |
| 7095 | 2.423967476 | down | 0.0000666 | 0.000517244 | BG511652 | 329 | 31 | 56 | 55 | 1e−13 | |
| 7096 | 2.423667193 | down | 0.0000341 | 0.000326244 | BM567882 | 431 | 62 | 94 | 65 | 9e−35 | cell wall modification |
| 7097 | 2.423360199 | down | 0.001097726 | 0.00390199 | AI443164 | 508 | 114 | 160 | 71 | 1e−70 | |
| 7098 | 2.423303408 | down | 0.000218919 | 0.00120439 | BU084337 | 520 | 27 | 57 | 47 | 2e−06 | metabolism |
| 7099 | 2.422724971 | down | 0.000152697 | 0.000928323 | BG156885 | 247 | 14 | 50 | 28 | 1.9 | |
| 7100 | 2.422613338 | down | 0.0000154 | 0.000189178 | CA784199 | 700 | 168 | 216 | 77 | 6e−94 | glycolysis |
| 7101 | 2.422455677 | down | 0.0000055 | 0.0000962 | CF922505 | 575 | 86 | 101 | 85 | 5e−46 | small GTPase mediated signal |
| | | | | | | | | | | | transduction |
| 7102 | 2.422317607 | down | 0.0000342 | 0.000326244 | BU081340 | 540 | 15 | 27 | 55 | 0.055 | |
| 7103 | 2.422216726 | down | 0.0000145 | 0.00018131 | AI736119 | 349 | 13 | 42 | 30 | 3.2 | |
| 7104 | 2.421899698 | down | 0.00000699 | 0.000111171 | CD405968 | 613 | 47 | 67 | 70 | 5e−23 | |
| 7105 | 2.421688802 | down | 0.000144881 | 0.000894691 | BQ611057 | 445 | 120 | 141 | 85 | 7e−65 | selenocysteine incorporation |
| 7106 | 2.421311504 | down | 0.0000216 | 0.000236799 | BG653092 | 512 | 136 | 170 | 80 | 9e−76 | |
| 7107 | 2.421171208 | down | 0.000318522 | 0.001585821 | BI969785 | 686 | 121 | 168 | 72 | 3e−66 | |
| 7108 | 2.421107448 | down | 0.0000742 | 0.00055761 | BU765147 | 325 | 12 | 19 | 63 | 0.64 | |
| 7109 | 2.420855102 | down | 0.00000598 | 0.000101267 | AW756558 | 409 | 101 | 136 | 74 | 2e−54 | protein amino acid phosphorylation |
| 7110 | 2.420268344 | down | 0.0000635 | 0.000499996 | CA783226 | 752 | 187 | 245 | 76 | e−110 | electron transport |
| 7111 | 2.419515805 | down | 0.000354701 | 0.001710477 | BU546621 | 684 | 40 | 71 | 56 | 2e−17 | |
| 7112 | 2.419051495 | down | 0.0000104 | 0.000144328 | AW101495 | 188 | 11 | 30 | 36 | 1.9 | |
| 7113 | 2.418757364 | down | 0.00000326 | 0.0000691 | X92437.1 | | | | | | Cinnamic acid 4-hydroxylase (CYP73) |
| 7114 | 2.418226734 | down | 0.00000395 | 0.000078 | BF325280 | 568 | 167 | 189 | 88 | 1e−94 | |
| 7115 | 2.417856654 | down | 0.00000564 | 0.0000977 | CF808896 | 548 | 84 | 147 | 57 | 3e−50 | dicarboxylic acid transport |
| 7116 | 2.417571119 | down | 0.0000464 | 0.000403247 | BM092715 | 556 | 126 | 177 | 71 | 1e−64 | |
| 7117 | 2.417090019 | down | 0.0000496 | 0.000420793 | CA800257 | 611 | 18 | 69 | 26 | 4.9 | transport |
| 7118 | 2.416663875 | down | 0.000100525 | 0.000690119 | BU545315 | 577 | 67 | 127 | 52 | 6e−39 | proteolysis and peptidolysis |
| 7119 | 2.416661649 | down | 0.001938023 | 0.005852344 | BM525031 | 555 | 78 | 183 | 42 | 7e−35 | regulation of transcription\, DNA- |
| | | | | | | | | | | | dependent |
| 7120 | 2.416441787 | down | 0.000373823 | 0.001778071 | BU547276 | 599 | 80 | 114 | 70 | 2e−44 | electron transport |
| 7121 | 2.4155374 | down | 0.000350037 | 0.001692908 | BG315974 | 570 | 74 | 123 | 60 | 5e−34 | protein amino acid phosphorylation |
| 7122 | 2.41512884 | down | 0.0000106 | 0.000145988 | BE609257 | 362 | 18 | 35 | 51 | 5e−04 | nodulation |
| 7123 | 2.414954971 | down | 0.00000262 | 0.0000607 | AW349631 | 529 | 56 | 101 | 55 | 2e−23 | |
| 7124 | 2.414826235 | down | 0.00017145 | 0.001011434 | BE824263 | 563 | 63 | 190 | 33 | 6e−18 | |
| 7125 | 2.413952195 | down | 0.0000175 | 0.000205265 | BM525047 | 569 | 23 | 93 | 24 | 1.1 | |
| 7126 | 2.413940799 | down | 0.0000828 | 0.000602245 | AW831499 | 451 | 94 | 135 | 69 | 4e−51 | |
| 7127 | 2.413544852 | down | 0.000220966 | 0.001213311 | CF808208 | 560 | 121 | 159 | 76 | 1e−62 | |
| 7128 | 2.413246166 | down | 0.00000333 | 0.0000702 | CD409997 | 696 | 97 | 102 | 95 | 1e−52 | photosynthesis light harvesting |
| 7129 | 2.413194942 | down | 0.00000318 | 0.0000682 | AW351168 | 666 | 128 | 170 | 75 | 2e−72 | malate metabolism |
| 7130 | 2.412807415 | down | 0.00000656 | 0.000106759 | BE473613 | 515 | 115 | 162 | 70 | 1e−66 | cell wall modification |
| 7131 | 2.412763106 | down | 0.000152996 | 0.000929542 | CD407100 | 648 | 45 | 70 | 64 | 3e−23 | |
| 7132 | 2.411694376 | down | 0.000003 | 0.0000659 | CF806570 | 511 | 92 | 151 | 60 | 1e−50 | metabolism |
| 7133 | 2.411616219 | down | 0.000255242 | 0.001348218 | BE057470 | 489 | 51 | 71 | 71 | 2e−28 | |
| 7134 | 2.411605035 | down | 0.0000575 | 0.000467095 | BI786740 | 568 | 100 | 189 | 52 | 6e−52 | electron transport |
| 7135 | 2.411594626 | down | 0.00000574 | 0.0000986 | CD395419 | 686 | 42 | 49 | 85 | 1e−19 | protein biosynthesis |
| 7136 | 2.410913042 | down | 0.000033 | 0.000318518 | AI442768 | 455 | 25 | 43 | 58 | 1e−11 | |
| 7137 | 2.41056808 | down | 0.0000232 | 0.000249247 | BM887582 | 398 | 17 | 47 | 36 | 0.007 | dicarboxylic acid transport |
| 7138 | 2.409900314 | down | 0.000125836 | 0.000813488 | CF809240 | 569 | 10 | 16 | 62 | 4.3 | regulation of transcription |
| 7139 | 2.401228403 | down | 0.0000654 | 0.000509812 | CA820573 | 588 | 19 | 51 | 37 | 2.7 | |
| 7140 | 2.400973405 | down | 0.000444926 | 0.002018508 | BG652280 | 420 | 11 | 32 | 34 | 8.7 | |
| 7141 | 2.400833882 | down | 0.001720496 | 0.005386161 | CA784580 | 681 | 104 | 120 | 86 | 2e−55 | galactose metabolism |
| 7142 | 2.400801547 | down | 0.0000108 | 0.000147495 | BU762234 | 623 | 57 | 85 | 67 | 4e−28 | protein amino acid phosphorylation |
| 7143 | 2.400325507 | down | 0.000425957 | 0.001956997 | BI943959 | 391 | 38 | 63 | 60 | 1e−17 | proteolysis and peptidolysis |
| 7144 | 2.400285398 | down | 0.00000768 | 0.000117626 | CD406753 | 626 | 43 | 95 | 45 | 4e−13 | |
| 7145 | 2.400189944 | down | 0.0000271 | 0.000277579 | BQ611864 | 176 | 13 | 41 | 31 | 3.2 | cation transport |
| 7146 | 2.400153318 | down | 0.000034 | 0.000324982 | BQ741384 | 580 | 47 | 64 | 73 | 1e−21 | regulation of transcription |
| 7147 | 2.400111615 | down | 0.000135122 | 0.00085262 | BI971993 | 531 | 21 | 37 | 56 | 1e−07 | |
| 7148 | 2.399688467 | down | 0.00000699 | 0.000111171 | AW099549 | 422 | 13 | 35 | 37 | 1.8 | |
| 7149 | 2.399616153 | down | 0.00006 | 0.000480264 | BE022913 | 318 | 16 | 49 | 32 | 1.9 | DNA methylation |
| 7150 | 2.399418728 | down | 0.00000621 | 0.000103843 | CD405603 | 641 | 59 | 108 | 54 | 2e−21 | regulation of transcription\, DNA- |
| | | | | | | | | | | | dependent |
| 7151 | 2.399388174 | down | 0.001937804 | 0.005852344 | CA784580 | 681 | 104 | 120 | 86 | 2e−55 | galactose metabolism |
| 7152 | 2.399138871 | down | 0.0000135 | 0.000172562 | AW598141 | 361 | 16 | 58 | 27 | 0.68 | protein folding |
| 7153 | 2.399049144 | down | 0.000574306 | 0.002440069 | AW317206 | 404 | 24 | 27 | 88 | 5e−08 | |
| 7154 | 2.398776357 | down | 0.0000418 | 0.00037414 | BU548865 | 554 | 74 | 94 | 78 | 4e−38 | metabolism |
| 7155 | 2.397951316 | down | 0.0000676 | 0.000522289 | BI892849 | 537 | 150 | 179 | 83 | 1e−84 | glycolysis |
| 7156 | 2.397420218 | down | 0.000334547 | 0.001639351 | BI969715 | 705 | 85 | 187 | 45 | 2e−41 | |
| 7157 | 2.397342052 | down | 0.00000413 | 0.0000804 | AW459201 | 300 | 20 | 58 | 34 | 5.5 | |
| 7158 | 2.397068962 | down | 0.0000367 | 0.000342061 | BE329901 | 581 | 72 | 84 | 85 | 1e−36 | transport |
| 7159 | 2.396923056 | down | 0.00000215 | 0.0000534 | AW597990 | 398 | 63 | 65 | 96 | 1e−28 | transport |
| 7160 | 2.396571205 | down | 0.0000273 | 0.000278999 | AW570318 | 382 | 40 | 86 | 46 | 2e−18 | |
| 7161 | 2.396525337 | down | 0.00000269 | 0.0000614 | BE659221 | 763 | 24 | 90 | 26 | 0.22 | |
| 7162 | 2.396351788 | down | 0.0000166 | 0.00019805 | BF069364 | 419 | 114 | 139 | 82 | 3e−64 | |
| 7163 | 2.396079883 | down | 0.0000111 | 0.000150465 | CD398411 | 636 | 85 | 118 | 72 | 1e−41 | nucleosome assembly |
| 7164 | 2.394994058 | down | 0.00000974 | 0.000137671 | BQ079884 | 460 | 15 | 40 | 37 | 0.97 | |
| 7165 | 2.394957645 | down | 0.0000474 | 0.000408364 | BE659327 | 436 | 19 | 58 | 32 | 0.016 | apoptosis |
| 7166 | 2.394541348 | down | 0.0000187 | 0.000213975 | BG363326 | 369 | 7 | 18 | 38 | 2.8 | ubiquitin-dependent protein catabolism |
| 7167 | 2.393790558 | down | 0.0000265 | 0.00027346 | BG839918 | 758 | 177 | 221 | 80 | 4e−98 | |
| 7168 | 2.393096546 | down | 0.0000121 | 0.000159693 | BU762843 | 569 | 51 | 135 | 37 | 1e−20 | |
| 7169 | 2.392785283 | down | 0.000262475 | 0.001376149 | BI784636 | 576 | 89 | 150 | 59 | 2e−39 | |
| 7170 | 2.392547548 | down | 0.00000746 | 0.000115878 | BU927041 | 442 | 51 | 141 | 36 | 4e−12 | regulation of translation |
| 7171 | 2.39252302 | down | 0.00000308 | 0.0000669 | BU548283 | 771 | 88 | 169 | 52 | 3e−45 | |
| 7172 | 2.392495016 | down | 0.0000358 | 0.000336577 | BI945133 | 322 | 18 | 49 | 36 | 0.075 | regulation of transcription\, DNA- |
| | | | | | | | | | | | dependent |
| 7173 | 2.392197568 | down | 0.000161658 | 0.000969175 | BE658252 | 713 | 39 | 49 | 79 | 2e−20 | |
| 7174 | 2.3921865 | down | 0.000393982 | 0.001847485 | BE659788 | 437 | 15 | 41 | 36 | 0.30 | |
| 7175 | 2.391701634 | down | 0.000159516 | 0.000960162 | CD413982 | 461 | 12 | 29 | 41 | 0.58 | |
| 7176 | 2.391565434 | down | 0.0000383 | 0.000352083 | BM528510 | 574 | 34 | 111 | 30 | 0.047 | apoptosis |
| 7177 | 2.391464973 | down | 0.0000061 | 0.000102546 | BI973117 | 420 | 51 | 103 | 49 | 1e−26 | electron transport |
| 7178 | 2.390934609 | down | 0.00000201 | 0.0000511 | CD415362 | 665 | 124 | 171 | 72 | 6e−69 | |
| 7179 | 2.390634625 | down | 0.00034355 | 0.001669549 | AW424283 | 445 | 22 | 30 | 73 | 8e−07 | |
| 7180 | 2.390532523 | down | 0.0000126 | 0.000164229 | BG650814 | 523 | 58 | 90 | 64 | 2e−28 | |
| 7181 | 2.389853601 | down | 0.0000496 | 0.000420793 | BQ610693 | 492 | 51 | 145 | 35 | 2e−13 | |
| 7182 | 2.389833456 | down | 0.0000209 | 0.00023095 | BE190157 | 615 | 85 | 203 | 41 | 5e−35 | |
| 7183 | 2.389103246 | down | 0.0000939 | 0.00065922 | BI700723 | 571 | 100 | 160 | 62 | 5e−57 | protein folding |
| 7184 | 2.389047665 | down | 0.000221969 | 0.001216897 | BE059235 | 435 | 24 | 42 | 57 | 3e−05 | |
| 7185 | 2.388935436 | down | 0.00000437 | 0.0000834 | BE824093 | 534 | 70 | 127 | 55 | 2e−35 | glycolysis |
| 7186 | 2.388860161 | down | 0.0000131 | 0.00016924 | CD398783 | 368 | 11 | 11 | 100 | 1.7 | transport |
| 7187 | 2.387253195 | down | 0.0000675 | 0.000521956 | CD395987 | 611 | 111 | 142 | 78 | 2e−61 | D-ribose metabolism |
| 7188 | 2.387133277 | down | 0.00000335 | 0.0000706 | BQ611546 | 322 | 12 | 21 | 57 | 0.64 | DNA recombination |
| 7189 | 2.387055345 | down | 0.0000121 | 0.000159627 | BQ651726 | 517 | 143 | 167 | 85 | 3e−80 | aromatic amino acid family biosynthesis |
| 7190 | 2.386958041 | down | 0.00002 | 0.000223756 | BQ080898 | 464 | 15 | 26 | 57 | 6e−04 | salicylic acid mediated signaling |
| | | | | | | | | | | | pathway (systemic acquired resistance) |
| 7191 | 2.386920853 | down | 0.000199401 | 0.001125633 | AW706022 | 578 | 17 | 26 | 65 | 3e−05 | nitrogen fixation |
| 7192 | 2.386664419 | down | 0.0000284 | 0.000286506 | BQ610576 | 529 | 46 | 65 | 70 | 1e−22 | |
| 7193 | 2.386654234 | down | 0.0000253 | 0.0002648 | BE023083 | 468 | 61 | 71 | 85 | 2e−32 | megasporogenesis |
| 7194 | 2.386648171 | down | 0.000163391 | 0.00097527 | BQ785154 | 226 | 11 | 28 | 39 | 3.2 | |
| 7195 | 2.385956875 | down | 0.0000122 | 0.000160374 | BG725838 | 515 | 28 | 51 | 54 | 3e−10 | aromatic compound metabolism |
| 7196 | 2.385285448 | down | 0.00000418 | 0.000081 | BI497954 | 362 | 22 | 40 | 55 | 8e−08 | |
| 7197 | 2.385112721 | down | 0.00000446 | 0.0000844 | BI945636 | 488 | 14 | 32 | 43 | 1.9 | protein amino acid prenylation |
| 7198 | 2.384226344 | down | 0.0000196 | 0.000221197 | BU547281 | 561 | 88 | 124 | 70 | 7e−46 | regulation of transcription\, DNA- |
| | | | | | | | | | | | dependent |
| 7199 | 2.383540586 | down | 0.0000346 | 0.000329807 | AW310082 | 468 | 32 | 102 | 31 | 3e−04 | ossification |
| 7200 | 2.383410334 | down | 0.00255778 | 0.007122625 | AW780869 | 335 | 22 | 53 | 41 | 0.007 | response to light |
| 7201 | 2.383261157 | down | 0.00000687 | 0.000109898 | BG653036 | 525 | 129 | 174 | 74 | 3e−72 | protein amino acid phosphorylation |
| 7202 | 2.383160434 | down | 0.001529864 | 0.00494656 | BE474155 | 494 | 103 | 165 | 62 | 6e−58 | carbohydrate metabolism |
| 7203 | 2.382721661 | down | 0.000382 | 0.00180759 | BM731847 | 557 | 169 | 185 | 91 | e−100 | protein biosynthesis |
| 7204 | 2.382597026 | down | 0.000174717 | 0.001024122 | AW348400 | 469 | 78 | 78 | 100 | 6e−38 | nucleosome assembly |
| 7205 | 2.382577323 | down | 0.000738491 | 0.002915057 | BG045664 | 558 | 135 | 151 | 89 | 5e−76 | galactose metabolism |
| 7206 | 2.382469955 | down | 0.0000507 | 0.00042706 | AW396856 | 406 | 84 | 119 | 70 | 1e−42 | |
| 7207 | 2.382438021 | down | 0.00000809 | 0.000121456 | BM178125 | 420 | 26 | 40 | 65 | 1e−05 | protein amino acid phosphorylation |
| 7208 | 2.381962644 | down | 0.0000148 | 0.000184109 | BE021411 | 440 | 90 | 126 | 71 | 3e−47 | |
| 7209 | 2.319740155 | down | 0.0000572 | 0.000465419 | BQ785950 | 524 | 102 | 150 | 68 | 2e−51 | regulation of transcription\, DNA- |
| | | | | | | | | | | | dependent |
| 7210 | 2.319668311 | down | 0.00000805 | 0.000121099 | BQ094677 | 549 | 113 | 176 | 64 | 8e−62 | |
| 7211 | 2.31939595 | down | 0.000119518 | 0.0007821 | AW395106 | 324 | 16 | 66 | 24 | 2.4 | |
| 7212 | 2.31930338 | down | 0.0000157 | 0.00019158 | BU547000 | 695 | 98 | 153 | 64 | 5e−50 | cell growth and/or maintenance |
| 7213 | 2.31912539 | down | 0.000151207 | 0.000921618 | BE822470 | 765 | 90 | 180 | 50 | 5e−48 | cell growth and/or maintenance |
| 7214 | 2.3189509 | down | 0.000349362 | 0.001691227 | BG510614 | 503 | 54 | 70 | 77 | 5e−28 | |
| 7215 | 2.318382944 | down | 0.000014 | 0.000176901 | CD414072 | 594 | 101 | 121 | 83 | 1e−56 | protein amino acid phosphorylation |
| 7216 | 2.318282178 | down | 0.0000367 | 0.000341964 | AW351023 | 372 | 40 | 59 | 67 | 2e−19 | signal transduction |
| 7217 | 2.318229101 | down | 0.0000176 | 0.000205986 | CA801733 | 616 | 113 | 182 | 62 | 7e−60 | |
| 7218 | 2.318108428 | down | 0.000130721 | 0.000834085 | CA802683 | 297 | 13 | 30 | 43 | 2.5 | |
| 7219 | 2.318064314 | down | 0.000047 | 0.000406543 | BU766031 | 488 | 78 | 122 | 63 | 1e−41 | |
| 7220 | 2.317401697 | down | 0.000205772 | 0.001151089 | CF806931 | 710 | 133 | 157 | 84 | 7e−68 | ossification |
| 7221 | 2.31736461 | down | 0.00000893 | 0.000129457 | CD414949 | 605 | 25 | 69 | 36 | 3e−05 | phosphoenolpyruvate-dependent sugar |
| | | | | | | | | | | | phosphotransferase system |
| 7222 | 2.317285079 | down | 0.00000991 | 0.00013935 | BF426105 | 540 | 36 | 54 | 66 | 3e−14 | regulation of transcription\, DNA- |
| | | | | | | | | | | | dependent |
| 7223 | 2.317178647 | down | 0.0000348 | 0.000330641 | AI959958 | 457 | 24 | 65 | 36 | 0.051 | |
| 7224 | 2.316939412 | down | 0.0000187 | 0.000213975 | BE821686 | 683 | 71 | 177 | 40 | 1e−28 | |
| 7225 | 2.31687189 | down | 0.0000866 | 0.000622251 | BM526895 | 548 | 77 | 135 | 57 | 1e−34 | regulation of transcription\, DNA- |
| | | | | | | | | | | | dependent |
| 7226 | 2.316639999 | down | 0.000401694 | 0.001874371 | BQ576403 | 456 | 35 | 107 | 32 | 1e−17 | regulation of transcription\, DNA- |
| | | | | | | | | | | | dependent |
| 7227 | 2.316559381 | down | 0.0000362 | 0.000339187 | BG155089 | 669 | 174 | 220 | 79 | 4e−99 | Nitrate reductase (NiR), electron |
| | | | | | | | | | | | transport |
| 7228 | 2.316501515 | down | 0.0000417 | 0.000373841 | BG551629 | 421 | 37 | 66 | 56 | 7e−13 | |
| 7229 | 2.316442798 | down | 0.000243239 | 0.001301884 | BE611202 | 410 | 94 | 136 | 69 | 9e−53 | |
| 7230 | 2.316294475 | down | 0.00000475 | 0.0000878 | BM519738 | 518 | 75 | 162 | 46 | 2e−31 | |
| 7231 | 2.316288917 | down | 0.0000249 | 0.000262747 | BU083811 | 554 | 111 | 148 | 75 | 3e−58 | |
| 7232 | 2.315763821 | down | 0.0000208 | 0.000230551 | BU761319 | 626 | 108 | 134 | 80 | 1e−55 | nucleosome assembly |
| 7233 | 2.315514928 | down | 0.0000493 | 0.000418849 | BI945484 | 526 | 81 | 160 | 50 | 4e−42 | protein amino acid phosphorylation |
| 7234 | 2.315388707 | down | 0.000600784 | 0.00251283 | BI426579 | 499 | 8 | 14 | 57 | 9.8 | |
| 7235 | 2.381267363 | down | 0.0000138 | 0.000174776 | AW348752 | 635 | 15 | 24 | 62 | 0.005 | |
| 7236 | 2.380940214 | down | 0.0000598 | 0.000478828 | CF805760 | 517 | 109 | 171 | 63 | 1e−57 | electron transport |
| 7237 | 2.380329577 | down | 0.0000173 | 0.000203923 | AI442912 | 610 | 85 | 103 | 82 | 2e−44 | protein biosynthesis |
| 7238 | 2.379322246 | down | 0.0000246 | 0.000259972 | AW459541 | 619 | 100 | 204 | 49 | 4e−50 | metabolism |
| 7239 | 2.378998097 | down | 0.00000274 | 0.000062 | AW348658 | 631 | 32 | 80 | 40 | 9e−13 | protein amino acid phosphorylation |
| 7240 | 2.378966099 | down | 0.0000177 | 0.000207104 | BM307174 | 563 | 50 | 86 | 58 | 4e−23 | |
| 7241 | 2.378891353 | down | 0.00000591 | 0.000100379 | BM731611 | | | | | | |
| 7242 | 2.378535875 | down | 0.0000448 | 0.000393074 | BG507829 | 399 | 31 | 57 | 54 | 7e−12 | |
| 7243 | 2.37788289 | down | 0.0000215 | 0.000236118 | CD405919 | 472 | 74 | 87 | 85 | 3e−43 | transport |
| 7244 | 2.377584772 | down | 0.000194976 | 0.001105992 | BE473997 | 486 | 48 | 122 | 39 | 3e−14 | |
| 7245 | 2.37696135 | down | 0.0000347 | 0.000330144 | CA819576 | 403 | 11 | 33 | 33 | 2.7 | regulation of transcription\, DNA- |
| | | | | | | | | | | | dependent |
| 7246 | 2.376562705 | down | 0.000419002 | 0.001933784 | CA802547 | 333 | 12 | 34 | 35 | 5.5 | |
| 7247 | 2.37608814 | down | 0.0000314 | 0.000307633 | BI700218 | 576 | 145 | 191 | 75 | 8e−82 | transport |
| 7248 | 2.376028911 | down | 0.00000265 | 0.0000609 | CF807539 | 642 | 70 | 146 | 47 | 6e−30 | |
| 7249 | 2.375883089 | down | 0.0000746 | 0.000559558 | BQ627738 | 317 | 13 | 25 | 52 | 3.2 | protein amino acid phosphorylation |
| 7250 | 2.375437783 | down | 0.000229883 | 0.001248204 | BM522949 | 421 | 27 | 36 | 75 | 2e−09 | cation transport |
| 7251 | 2.375408175 | down | 0.00000186 | 0.0000486 | AW569658 | 450 | 35 | 48 | 72 | 2e−15 | response to stress |
| 7252 | 2.375221797 | down | 0.0000108 | 0.00014756 | BG839298 | 563 | 16 | 55 | 29 | 7.2 | protein amino acid phosphorylation |
| 7253 | 2.374810071 | down | 0.000552608 | 0.002369149 | BQ080890 | 524 | 70 | 107 | 65 | 3e−33 | N-terminal protein myristoylation |
| 7254 | 2.374545952 | down | 0.00000762 | 0.000117192 | BI426895 | 486 | 66 | 128 | 51 | 6e−28 | metabolism |
| 7255 | 2.374490841 | down | 0.000199879 | 0.00112768 | BM188504 | 517 | 128 | 172 | 74 | 1e−67 | transport |
| 7256 | 2.373842342 | down | 0.000590685 | 0.002485792 | CD392010 | 523 | 26 | 38 | 68 | 5e−11 | regulation of transcription |
| 7257 | 2.373259425 | down | 0.0000608 | 0.000484479 | CD404769 | 467 | 27 | 35 | 77 | 6e−09 | metabolism |
| 7258 | 2.37246788 | down | 0.0000347 | 0.000330144 | CA799335 | 612 | 123 | 204 | 60 | 6e−69 | transport |
| 7259 | 2.372192755 | down | 0.0000125 | 0.00016339 | AW310570 | 472 | 38 | 79 | 48 | 3e−12 | |
| 7260 | 2.371781969 | down | 0.0000228 | 0.000246214 | BE820579 | 757 | 91 | 165 | 55 | 1e−43 | |
| 7261 | 2.37177096 | down | 0.000112911 | 0.000749439 | CD399983 | 355 | 13 | 35 | 37 | 0.87 | |
| 7262 | 2.371076294 | down | 0.0000196 | 0.000221197 | CK606454 | | | | | | |
| 7263 | 2.371049669 | down | 0.00000685 | 0.000109774 | BU550868 | 613 | 124 | 160 | 77 | 2e−76 | |
| 7264 | 2.370898265 | down | 0.0000163 | 0.000196427 | AW102238 | 334 | 13 | 31 | 41 | 4.2 | |
| 7265 | 2.370710628 | down | 0.0000518 | 0.000433187 | AW310981 | 473 | 14 | 36 | 38 | 1.8 | electron transport |
| 7266 | 2.370368379 | down | 0.00000517 | 0.0000929 | CA802301 | 683 | 207 | 220 | 94 | e−118 | |
| 7267 | 2.370266537 | down | 0.00000448 | 0.0000847 | AW310570 | 472 | 38 | 79 | 48 | 3e−12 | |
| 7268 | 2.370066423 | down | 0.0000039 | 0.0000776 | CA935779 | 576 | 130 | 167 | 77 | 1e−75 | |
| 7269 | 2.369988581 | down | 0.00000971 | 0.000137423 | BU761668 | 459 | 110 | 137 | 80 | 1e−62 | metabolism |
| 7270 | 2.369401645 | down | 0.000332784 | 0.001632838 | BG045664 | 558 | 135 | 151 | 89 | 5e−76 | galactose metabolism |
| 7271 | 2.360178472 | down | 0.000302627 | 0.001527305 | BU964565 | 581 | 98 | 135 | 72 | 4e−48 | proteolysis and peptidolysis |
| 7272 | 2.359887043 | down | 0.00000733 | 0.000114495 | BE658167 | 342 | 11 | 38 | 28 | 1.9 | ossification |
| 7273 | 2.359395585 | down | 0.0000229 | 0.000246966 | BI973058 | 595 | 135 | 147 | 91 | 5e−77 | |
| 7274 | 2.359317346 | down | 0.0000447 | 0.0003925 | CF807694 | 560 | 103 | 111 | 92 | 4e−58 | protein amino acid phosphorylation |
| 7275 | 2.359302859 | down | 0.0000399 | 0.000361079 | AW755326 | 333 | 13 | 28 | 46 | 9.5 | electron transport |
| 7276 | 2.35924106 | down | 0.0000168 | 0.000199387 | BU762195 | 628 | 138 | 209 | 66 | 9e−77 | protein amino acid phosphorylation |
| 7277 | 2.359119788 | down | 0.000280932 | 0.001445946 | BF008821 | 465 | 35 | 61 | 57 | 5e−16 | regulation of nitrogen utilization |
| 7278 | 2.358959771 | down | 0.0000132 | 0.000170303 | S46989.1 | | | | | | Chalcone synthase [soybeans, mRNA, |
| | | | | | | | | | | | 1119 nt] |
| 7279 | 2.358892938 | down | 0.00000902 | 0.000130472 | CF922008 | 643 | 72 | 142 | 50 | 3e−33 | response to sucrose stimulus |
| 7280 | 2.358800632 | down | 0.0000533 | 0.000442568 | AF506517.1 | | | | | | receptor-like kinase RHG1 |
| 7281 | 2.358029899 | down | 0.000722256 | 0.002867524 | AF244890.1 | | | | | | receptor-like protein kinase 3 |
| 7282 | 2.357610356 | down | 0.0000914 | 0.000647147 | BM092247 | | | | | | |
| 7283 | 2.357298798 | down | 0.0000183 | 0.000210869 | BU765586 | 596 | 22 | 46 | 47 | 0.002 | |
| 7284 | 2.356574743 | down | 0.000063 | 0.000497003 | BU551240 | 749 | 44 | 75 | 58 | 2e−20 | |
| 7285 | 2.356429519 | down | 0.0000526 | 0.000438086 | AW279120 | 377 | 80 | 105 | 76 | 5e−46 | |
| 7286 | 2.356417817 | down | 0.0000237 | 0.000252878 | BU547766 | 500 | 40 | 54 | 74 | 6e−16 | regulation of transcription |
| 7287 | 2.356404123 | down | 0.00000743 | 0.000115599 | BE819876 | 367 | 11 | 17 | 64 | 0.32 | |
| 7288 | 2.356324025 | down | 0.0000598 | 0.000478997 | BI970982 | 466 | 30 | 76 | 39 | 6e−11 | threonine catabolism |
| 7289 | 2.356075958 | down | 0.000257873 | 0.001359064 | BG044238 | 112 | 15 | 27 | 55 | 0.005 | electron transport |
| 7290 | 2.355716575 | down | 0.000004 | 0.0000787 | AW102252 | 155 | 7 | 20 | 35 | 9.2 | |
| 7291 | 2.355207145 | down | 0.00000438 | 0.0000836 | BU550307 | 640 | 119 | 179 | 66 | 2e−65 | electron transport |
| 7292 | 2.355066039 | down | 0.0000194 | 0.000220022 | BQ253025 | 594 | 12 | 31 | 38 | 3.5 | |
| 7293 | 2.354916909 | down | 0.0000277 | 0.000281619 | BQ741480 | 593 | 95 | 126 | 75 | 7e−55 | |
| 7294 | 2.354252288 | down | 0.00000403 | 0.000079 | AW432908 | 482 | 32 | 55 | 58 | 5e−12 | protein amino acid phosphorylation |
| 7295 | 2.353827643 | down | 0.000249385 | 0.001325472 | CA853093 | 295 | 37 | 80 | 46 | 7e−11 | metabolism |
| 7296 | 2.353248048 | down | 0.00000896 | 0.000129711 | CF808835 | 283 | 48 | 54 | 88 | 3e−25 | electron transport |
| 7297 | 2.352868775 | down | 0.00000304 | 0.0000665 | BQ785970 | 546 | 32 | 45 | 71 | 2e−12 | |
| 7298 | 2.352605313 | down | 0.000550043 | 0.002360984 | BI424988 | 575 | 52 | 99 | 52 | 1e−25 | |
| 7299 | 2.352371337 | down | 0.00000881 | 0.000128368 | CD392345 | 635 | 94 | 119 | 78 | 3e−47 | protein biosynthesis |
| 7300 | 2.351661883 | down | 0.0000588 | 0.000474309 | CD395529 | 450 | 37 | 73 | 50 | 7e−12 | |
| 7301 | 2.3513841 | down | 0.000263255 | 0.001377775 | BE820317 | 563 | 39 | 70 | 55 | 4e−14 | |
| 7302 | 2.350813613 | down | 0.0000516 | 0.000432237 | AW397591 | 311 | 10 | 21 | 47 | 4.2 | |
| 7303 | 2.35079822 | down | 0.0000523 | 0.000436167 | BQ453695 | 498 | 53 | 87 | 60 | 2e−27 | |
| 7304 | 2.350717563 | down | 0.0000158 | 0.000192003 | AW101961 | 207 | 16 | 49 | 32 | 5.6 | transport |
| 7305 | 2.350332322 | down | 0.0000224 | 0.000243492 | BU548211 | 700 | 105 | 139 | 75 | 1e−57 | protein amino acid phosphorylation |
| 7306 | 2.3500199 | down | 0.0000268 | 0.000275269 | BM188920 | 427 | 10 | 41 | 24 | 3.1 | |
| 7307 | 2.34997297 | down | 0.0000599 | 0.000479746 | BG510539 | 287 | 14 | 50 | 28 | 1.4 | |
| 7308 | 2.349572575 | down | 0.00000971 | 0.000137423 | BU577819 | 408 | 109 | 135 | 80 | 5e−59 | transport |
| 7309 | 2.349556868 | down | 0.0000153 | 0.000188494 | BM568073 | 284 | 15 | 37 | 40 | 0.13 | response to pathogenic bacteria |
| 7310 | 2.349347468 | down | 0.0000472 | 0.000407657 | BU545509 | 526 | 48 | 70 | 68 | 3e−22 | |
| 7311 | 2.349317946 | down | 0.00000452 | 0.0000851 | CF922355 | 720 | 144 | 182 | 79 | 2e−80 | cell differentiation |
| 7312 | 2.34928454 | down | 0.001138711 | 0.00399883 | BI701521 | 564 | 141 | 188 | 75 | 5e−83 | |
| 7313 | 2.348895379 | down | 0.0000135 | 0.000172562 | AW397813 | 561 | 101 | 163 | 61 | 4e−57 | protein amino acid phosphorylation |
| 7314 | 2.348867582 | down | 0.000131562 | 0.000838031 | AW831580 | 529 | 71 | 154 | 46 | 8e−38 | |
| 7315 | 2.348706962 | down | 0.0000719 | 0.000544985 | CA853797 | 411 | 94 | 126 | 74 | 9e−52 | response to light |
| 7316 | 2.348213922 | down | 0.00000748 | 0.000116042 | BI970266 | 752 | 86 | 135 | 63 | 4e−47 | biological_process unknown |
| 7317 | 2.347768942 | down | 0.000773905 | 0.003019988 | CF808757 | 574 | 162 | 190 | 85 | 9e−91 | ferredoxin-dependent glutamate |
| | | | | | | | | | | | synthase, photorespiration |
| 7318 | 2.34776235 | down | 0.00057278 | 0.002435513 | BI426746 | 568 | 128 | 189 | 67 | 2e−74 | |
| 7319 | 2.347758783 | down | 0.00000371 | 0.0000751 | BE917603 | 571 | 67 | 131 | 51 | 1e−27 | protein amino acid phosphorylation |
| 7320 | 2.347577118 | down | 0.001631083 | 0.005187415 | AW508411 | 629 | 186 | 209 | 88 | e−111 | adenosine 5′-phosphosulfate reductase, |
| | | | | | | | | | | | sulfate assimilation |
| 7321 | 2.34678198 | down | 0.0000443 | 0.00039025 | AW203448 | 363 | 12 | 30 | 40 | 6.0 | proteolysis and peptidolysis |
| 7322 | 2.290641503 | down | 0.000059 | 0.000475076 | BI967234 | 537 | 22 | 34 | 64 | 3e−06 | |
| 7323 | 2.29043435 | down | 0.0000416 | 0.000373018 | BU081089 | 612 | 161 | 204 | 78 | 8e−93 | |
| 7324 | 2.289894456 | down | 0.000015 | 0.00018613 | BE819876 | 367 | 11 | 17 | 64 | 0.32 | |
| 7325 | 2.289797798 | down | 0.000166061 | 0.000987387 | BM528163 | 541 | 92 | 180 | 51 | 2e−46 | |
| 7326 | 2.289782902 | down | 0.0000178 | 0.000207266 | CD403460 | 472 | 30 | 46 | 65 | 2e−10 | |
| 7327 | 2.289585481 | down | 0.0000154 | 0.000189375 | BI969658 | 692 | 57 | 122 | 46 | 4e−25 | response to oxidative stress |
| 7328 | 2.289408994 | down | 0.0000282 | 0.000285142 | CF809053 | 668 | 89 | 156 | 57 | 5e−49 | electron transport |
| 7329 | 2.289318145 | down | 0.000102784 | 0.000700911 | BE807371 | 562 | 42 | 108 | 38 | 5e−12 | |
| 7330 | 2.288797352 | down | 0.0000922 | 0.000650503 | CD412403 | 606 | 45 | 72 | 62 | 3e−17 | electron transport |
| 7331 | 2.288647667 | down | 0.0000448 | 0.000393017 | AW459469 | 502 | 19 | 41 | 46 | 2e−04 | |
| 7332 | 2.288383465 | down | 0.000146189 | 0.000900256 | BI322150 | 517 | 84 | 136 | 61 | 3e−39 | |
| 7333 | 2.288340559 | down | 0.001014863 | 0.003686438 | CF807455 | 733 | 157 | 207 | 75 | 1e−89 | glycolysis |
| 7334 | 2.288194938 | down | 0.0000651 | 0.000508666 | BM521060 | 534 | 98 | 161 | 60 | 8e−56 | transport |
| 7335 | 2.28804272 | down | 0.00000749 | 0.000116042 | BU545678 | 587 | 19 | 57 | 33 | 0.11 | |
| 7336 | 2.287940294 | down | 0.0000282 | 0.000285222 | CA783500 | 744 | 179 | 181 | 98 | e−100 | small GTPase mediated signal |
| | | | | | | | | | | | transduction |
| 7337 | 2.287870576 | down | 0.0000217 | 0.000237472 | BI320985 | 219 | 12 | 26 | 46 | 2.5 | |
| 7338 | 2.346496088 | down | 0.000222403 | 0.001218566 | BU082164 | 467 | 43 | 65 | 66 | 3e−22 | protein amino acid phosphorylation |
| 7339 | 2.346305657 | down | 0.0000179 | 0.000207836 | AW350142 | 720 | 71 | 133 | 53 | 4e−29 | proteolysis and peptidolysis |
| 7340 | 2.346065716 | down | 0.000131038 | 0.000835824 | BE802684 | 419 | 39 | 64 | 60 | 1e−16 | protein amino acid phosphorylation |
| 7341 | 2.345858572 | down | 0.00000276 | 0.0000622 | CA784132 | 637 | 84 | 217 | 38 | 2e−28 | |
| 7342 | 2.345819494 | down | 0.00010617 | 0.000716794 | CD395877 | 467 | 11 | 31 | 35 | 3.9 | electron transport |
| 7343 | 2.345687895 | down | 0.0000825 | 0.000601261 | BG650304 | 294 | 11 | 20 | 55 | 9.5 | apoptosis |
| 7344 | 2.34560758 | down | 0.0000392 | 0.000357317 | BI971571 | 761 | 88 | 140 | 62 | 4e−51 | |
| 7345 | 2.345064227 | down | 0.000151255 | 0.000921618 | CA786051 | 437 | 17 | 50 | 34 | 0.18 | |
| 7346 | 2.344915379 | down | 0.0000327 | 0.00031605 | BI471946 | 499 | 57 | 128 | 44 | 4e−25 | |
| 7347 | 2.344825553 | down | 0.0000107 | 0.000146882 | CD397751 | 529 | 53 | 71 | 74 | 3e−25 | |
| 7348 | 2.344731728 | down | 0.0000717 | 0.000544809 | BQ253787 | 329 | 9 | 28 | 32 | 3.3 | |
| 7349 | 2.344125939 | down | 0.0000124 | 0.000163085 | CA802338 | 390 | 13 | 34 | 38 | 3.3 | protein amino acid phosphorylation |
| 7350 | 2.343662871 | down | 0.0000395 | 0.000359005 | BG316282 | 499 | 88 | 108 | 81 | 7e−46 | electron transport |
| 7351 | 2.34346918 | down | 0.0000182 | 0.000209952 | CF807103 | 269 | 16 | 33 | 48 | 5.5 | MRNA from stress-induced gene (H4) |
| 7352 | 2.343365432 | down | 0.0000473 | 0.000408196 | CA853724 | 474 | 67 | 87 | 77 | 1e−33 | biosynthesis |
| 7353 | 2.34303812 | down | 0.000725788 | 0.002877902 | BQ786370 | 591 | 134 | 201 | 66 | 3e−69 | glycolysis |
| 7354 | 2.342995386 | down | 0.00000354 | 0.0000729 | AW349830 | 738 | 55 | 120 | 45 | 3e−16 | |
| 7355 | 2.342529748 | down | 0.0000593 | 0.000476504 | CD414873 | 509 | 15 | 36 | 41 | 2.7 | metabolism |
| 7356 | 2.342223356 | down | 0.0000439 | 0.000387895 | CA819791 | 285 | 11 | 27 | 40 | 4.1 | metabolism |
| 7357 | 2.341601022 | down | 0.0000428 | 0.000381016 | BU548218 | 617 | 76 | 117 | 64 | 6e−36 | |
| 7358 | 2.340289587 | down | 0.0000236 | 0.000252555 | BU549436 | 348 | 16 | 50 | 32 | 1.4 | |
| 7359 | 2.340045423 | down | 0.0000214 | 0.000234853 | BE657667 | 670 | 56 | 203 | 27 | 2e−10 | |
| 7360 | 2.339589104 | down | 0.000197193 | 0.001115707 | BI426451 | 552 | 121 | 130 | 93 | 5e−70 | microtubule-based process |
| 7361 | 2.339547236 | down | 0.0000187 | 0.000213655 | CK606415 | 810 | 122 | 209 | 58 | 8e−63 | |
| 7362 | 2.33948561 | down | 0.0000508 | 0.000427573 | BU082733 | 549 | 84 | 191 | 43 | 1e−42 | |
| 7363 | 2.339395511 | down | 0.0000625 | 0.000494496 | AW756084 | 267 | 32 | 76 | 42 | 1e−08 | G1 phase of mitotic cell cycle |
| 7364 | 2.338696917 | down | 0.00000339 | 0.000071 | CF807294 | 710 | 191 | 204 | 93 | e−109 | |
| 7365 | 2.338325736 | down | 0.000233418 | 0.001263806 | AF244889.1 | | | | | | receptor-like protein kinase 2 |
| 7366 | 2.338148557 | down | 0.000161566 | 0.000968934 | BM092288 | 313 | 26 | 34 | 76 | 4e−10 | protein folding |
| 7367 | 2.338072463 | down | 0.00024687 | 0.00131601 | AW309518 | 598 | 34 | 69 | 49 | 4e−12 | |
| 7368 | 2.337595945 | down | 0.0000826 | 0.000601333 | AW351166 | 362 | 12 | 28 | 42 | 4.6 | peroxisome organization and biogenesis |
| 7369 | 2.337064346 | down | 0.0000078 | 0.000118925 | AI496328 | 442 | 49 | 99 | 49 | 1e−18 | hypersensitive response |
| 7370 | 2.336928163 | down | 0.0000121 | 0.000159301 | BE059367 | 417 | 42 | 63 | 66 | 8e−19 | lipid catabolism |
| 7371 | 2.336826317 | down | 0.00000252 | 0.0000592 | CD395352 | 523 | 28 | 51 | 54 | 4e−10 | |
| 7372 | 2.336535624 | down | 0.0000331 | 0.00031907 | BQ610438 | 391 | 18 | 37 | 48 | 1.5 | regulation of transcription\, DNA- |
| | | | | | | | | | | | dependent |
| 7373 | 2.336233864 | down | 0.0000454 | 0.00039708 | AW350817 | 447 | 57 | 81 | 70 | 3e−24 | |
| 7374 | 2.336149084 | down | 0.0000021 | 0.0000527 | AI855595 | 202 | 19 | 48 | 39 | 0.83 | transcription initiation |
| 7375 | 2.336086012 | down | 0.000860302 | 0.003267636 | CK606208 | 820 | 74 | 130 | 56 | 4e−38 | Vegetative storage protien mRNA |
| | | | | | | | | | | | (VSP27), carbohydrate metabolism |
| 7376 | 2.3356696 | down | 0.00096425 | 0.003558255 | BU761432 | 590 | 19 | 58 | 32 | 0.92 | oligopeptide transport |
| 7377 | 2.335576941 | down | 0.0000272 | 0.000277579 | BE822397 | 618 | 109 | 179 | 60 | 4e−64 | |
| 7378 | 2.335409841 | down | 0.0000127 | 0.000165171 | BQ298286 | 316 | 12 | 18 | 66 | 2.4 | cation transport |
| 7379 | 2.335353759 | down | 0.000068 | 0.000524508 | BI893194 | 594 | 87 | 199 | 43 | 3e−46 | cell wall biosynthesis |
| 7380 | 2.335309208 | down | 0.0000117 | 0.000155724 | AW349633 | 733 | 77 | 144 | 53 | 1e−32 | |
| 7381 | 2.334983781 | down | 0.00000352 | 0.0000728 | BU550593 | 614 | 70 | 99 | 70 | 4e−41 | |
| 7382 | 2.334952044 | down | 0.000025 | 0.000266283 | CF806264 | 237 | 11 | 18 | 61 | 2.4 | regulation of transcription\, DNA- |
| | | | | | | | | | | | dependent |
| 7383 | 2.334815804 | down | 0.0000878 | 0.000628608 | CF805871 | 605 | 38 | 109 | 34 | 3e−14 | |
| 7384 | 2.334400328 | down | 0.00000724 | 0.000113774 | CF805866 | 652 | 120 | 221 | 54 | 2e−56 | regulation of transcription |
| 7385 | 2.334370271 | down | 0.000209145 | 0.001164587 | BM188324 | 602 | 128 | 179 | 71 | 2e−71 | regulation of transcription |
| 7386 | 2.333771785 | down | 0.000238924 | 0.001283657 | BI967711 | 623 | 78 | 183 | 42 | 3e−28 | regulation of transcription |
| 7387 | 2.333424823 | down | 0.0000535 | 0.000443203 | BI472180 | 589 | 112 | 194 | 57 | 6e−53 | transport |
| 7388 | 2.333266537 | down | 0.0000895 | 0.000636889 | AI438031 | 333 | 11 | 32 | 34 | 2.5 | |
| 7389 | 2.332831943 | down | 0.0000616 | 0.00048928 | BI785600 | 577 | 129 | 192 | 67 | 2e−74 | |
| 7390 | 2.332591075 | down | 0.000177859 | 0.001035924 | BE473658 | 649 | 53 | 80 | 66 | 7e−21 | |
| 7391 | 2.332428423 | down | 0.0000139 | 0.000176178 | AW306696 | 232 | 11 | 26 | 42 | 4.2 | proteolysis and peptidolysis |
| 7392 | 2.332318595 | down | 0.0000176 | 0.000205986 | AI441921 | 381 | 98 | 131 | 74 | 1e−48 | protein amino acid phosphorylation |
| 7393 | 2.331686798 | down | 0.0000186 | 0.000213297 | BE806780 | 543 | 140 | 178 | 78 | 1e−79 | cell differentiation |
| 7394 | 2.33121743 | down | 0.00000806 | 0.000121122 | BU764584 | 450 | 33 | 40 | 82 | 4e−12 | |
| 7395 | 2.330638208 | down | 0.00000887 | 0.000128818 | CF808588 | 492 | 67 | 89 | 75 | 6e−34 | |
| 7396 | 2.329633652 | down | 0.00000394 | 0.000078 | BU550179 | 688 | 29 | 52 | 55 | 7e−09 | |
| 7397 | 2.329526991 | down | 0.0000175 | 0.000205037 | BQ612821 | 440 | 17 | 61 | 27 | 3.4 | nucleosome assembly |
| 7398 | 2.329339326 | down | 0.0000386 | 0.000352911 | AW507722 | 523 | 145 | 173 | 83 | 1e−90 | |
| 7399 | 2.329156968 | down | 0.000323269 | 0.001600558 | BU547520 | 588 | 85 | 153 | 55 | 4e−41 | |
| 7400 | 2.329113799 | down | 0.0000497 | 0.000420922 | CF809126 | 557 | 119 | 184 | 64 | 5e−70 | |
| 7401 | 2.328996256 | down | 0.00000381 | 0.0000764 | BM093448 | 557 | 56 | 81 | 69 | 1e−24 | |
| 7402 | 2.328526854 | down | 0.0000785 | 0.000579497 | BU549480 | 217 | 14 | 38 | 36 | 4.2 | |
| 7403 | 2.325316292 | down | 0.0000268 | 0.000275227 | CA934968 | 187 | 10 | 30 | 33 | 5.5 | protein amino acid phosphorylation |
| 7404 | 2.323807777 | down | 0.000027 | 0.00027683 | CD393966 | 662 | 58 | 93 | 62 | 3e−30 | |
| 7405 | 2.323796421 | down | 0.000990515 | 0.003628125 | BM092500 | 560 | 137 | 189 | 72 | 3e−74 | |
| 7406 | 2.323492749 | down | 0.0000192 | 0.000218401 | BU549315 | 538 | 59 | 92 | 64 | 7e−29 | biosynthesis |
| 7407 | 2.323393579 | down | 0.0000437 | 0.000386645 | CF806786 | 711 | 160 | 207 | 77 | 5e−88 | plasmid partitioning (sensu Bacteria) |
| 7408 | 2.322941439 | down | 0.000593212 | 0.00249308 | AW202369 | 279 | 39 | 84 | 46 | 2e−11 | electron transport |
| 7409 | 2.322851455 | down | 0.00000302 | 0.0000662 | BG508345 | 384 | 24 | 31 | 77 | 6e−09 | regulation of transcription\, DNA- |
| | | | | | | | | | | | dependent |
| 7410 | 2.322843964 | down | 0.001376126 | 0.004579922 | AW306930 | 429 | 24 | 71 | 33 | 0.012 | |
| 7411 | 2.322774975 | down | 0.00000786 | 0.000119345 | CD397751 | 529 | 53 | 71 | 74 | 3e−25 | |
| 7412 | 2.322590153 | down | 0.000165172 | 0.000983347 | BM270534 | 417 | 80 | 119 | 67 | 1e−39 | |
| 7413 | 2.322456971 | down | 0.0000357 | 0.000335883 | AW705539 | 399 | 17 | 59 | 28 | 2.0 | |
| 7414 | 2.322169112 | down | 0.00000862 | 0.000126391 | CD401821 | 619 | 56 | 70 | 80 | 9e−29 | D-xylose metabolism |
| 7415 | 2.322123338 | down | 0.0000483 | 0.000413458 | BG405522 | 381 | 50 | 81 | 61 | 1e−24 | |
| 7416 | 2.321596406 | down | 0.0000346 | 0.00032949 | BU579354 | 423 | 88 | 116 | 75 | 1e−47 | transport |
| 7417 | 2.321160865 | down | 0.000032 | 0.000311368 | BI971182 | 219 | 17 | 57 | 29 | 7.2 | |
| 7418 | 2.320161833 | down | 0.00000645 | 0.000105861 | CD405955 | 640 | 90 | 166 | 54 | 3e−47 | protein amino acid phosphorylation |
| 7419 | 2.315386826 | down | 0.0000322 | 0.000312917 | BM885282 | 563 | 118 | 188 | 62 | 7e−67 | |
| 7420 | 2.315203317 | down | 0.000325671 | 0.001609483 | BI969927 | 696 | 114 | 150 | 76 | 1e−57 | nucleosome assembly |
| 7421 | 2.31508409 | down | 0.00000961 | 0.000136415 | BI426015 | 375 | 59 | 74 | 79 | 4e−33 | |
| 7422 | 2.314864875 | down | 0.000114775 | 0.000758723 | BM178289 | 499 | 22 | 45 | 48 | 3e−06 | electron transport |
| 7423 | 2.314759557 | down | 0.0000239 | 0.000254229 | AW351116 | 806 | 117 | 201 | 58 | 1e−66 | |
| 7424 | 2.314594609 | down | 0.0000145 | 0.000180904 | BI944238 | 187 | 13 | 33 | 39 | 7.1 | |
| 7425 | 2.314385203 | down | 0.0000154 | 0.00018894 | BI471295 | 566 | 76 | 178 | 42 | 9e−32 | |
| 7426 | 2.314142273 | down | 0.00000377 | 0.0000759 | CD415458 | 628 | 86 | 120 | 71 | 3e−45 | |
| 7427 | 2.314113207 | down | 0.0000223 | 0.000242122 | BU549701 | 544 | 24 | 58 | 41 | 7e−07 | |
| 7428 | 2.313479291 | down | 0.00000849 | 0.000125022 | BQ627697 | 356 | 29 | 40 | 72 | 2e−09 | biosynthesis |
| 7429 | 2.313418482 | down | 0.000537603 | 0.002319765 | CF805855 | 624 | 79 | 133 | 59 | 7e−34 | GH1 protein (GH1), gravitropism |
| 7430 | 2.313399992 | down | 0.0000133 | 0.000170634 | BI968028 | 763 | 47 | 84 | 55 | 2e−24 | |
| 7431 | 2.312974085 | down | 0.0005548 | 0.002377337 | BU760670 | 646 | 74 | 179 | 41 | 8e−31 | apoptosis |
| 7432 | 2.312395593 | down | 0.00000674 | 0.000108661 | BQ740987 | 599 | 82 | 202 | 40 | 9e−39 | |
| 7433 | 2.312256388 | down | 0.0000755 | 0.000564621 | CD416090 | 393 | 27 | 39 | 69 | 4e−11 | |
| 7434 | 2.312038958 | down | 0.0000243 | 0.000257122 | BU084397 | 420 | 121 | 138 | 87 | 1e−67 | transport |
| 7435 | 2.311716116 | down | 0.000221151 | 0.001213652 | AW133151 | 721 | 121 | 155 | 78 | 6e−64 | protein biosynthesis |
| 7436 | 2.311483969 | down | 0.000151927 | 0.000924641 | BI967716 | 223 | 14 | 50 | 28 | 7.2 | N-terminal protein amino acid |
| | | | | | | | | | | | acetylation |
| 7437 | 2.311444878 | down | 0.0000759 | 0.000566349 | BU547223 | 664 | 86 | 112 | 76 | 3e−48 | |
| 7438 | 2.310799713 | down | 0.002345401 | 0.006692964 | BM085417 | 569 | 169 | 189 | 89 | e−100 | primary cell wall biosynthesis (sensu |
| | | | | | | | | | | | Magnoliophyta) |
| 7439 | 2.31036134 | down | 0.0000973 | 0.000676347 | BI470370 | 574 | 113 | 191 | 59 | 5e−58 | biosynthesis |
| 7440 | 2.309449895 | down | 0.00000953 | 0.000135423 | CF806957 | 688 | 70 | 132 | 53 | 3e−29 | cell death |
| 7441 | 2.309361481 | down | 0.0000339 | 0.000324436 | BM519739 | 593 | 80 | 90 | 88 | 3e−46 | pectin biosynthesis |
| 7442 | 2.309085231 | down | 0.0000567 | 0.00046255 | BF070887 | 455 | 79 | 130 | 60 | 2e−42 | |
| 7443 | 2.308960983 | down | 0.0000854 | 0.000615933 | CA802726 | 247 | 13 | 39 | 33 | 1.9 | |
| 7444 | 2.308625603 | down | 0.0000355 | 0.000334527 | BU549896 | 293 | 21 | 52 | 40 | 0.002 | |
| 7445 | 2.308486365 | down | 0.00000527 | 0.0000938 | BI969421 | 780 | 74 | 173 | 42 | 4e−38 | |
| 7446 | 2.308462737 | down | 0.0000646 | 0.000506485 | CA937365 | 567 | 21 | 30 | 70 | 6e−07 | |
| 7447 | 2.308199067 | down | 0.000103882 | 0.000707099 | BE610326 | 279 | 11 | 38 | 28 | 0.84 | translational elongation |
| 7448 | 2.307421831 | down | 0.000127919 | 0.000821363 | AW277440 | 437 | 16 | 44 | 36 | 0.88 | |
| 7449 | 2.307297505 | down | 0.00000357 | 0.0000732 | AW349686 | 698 | 82 | 118 | 69 | 7e−38 | regulation of transcription |
| 7450 | 2.3068907 | down | 0.000901897 | 0.003388668 | AW349476 | 302 | 17 | 28 | 60 | 3e−04 | metal ion transport |
| 7451 | 2.306471598 | down | 0.000619403 | 0.002569142 | BE823928 | 411 | 18 | 36 | 50 | 0.008 | |
| 7452 | 2.306281365 | down | 0.000272643 | 0.00141334 | BI497615 | 471 | 35 | 112 | 31 | 2e−05 | |
| 7453 | 2.306208843 | down | 0.00078993 | 0.003066072 | BG238788 | 725 | 197 | 237 | 83 | e−111 | glycolysis |
| 7454 | 2.306093657 | down | 0.000108166 | 0.000727045 | AW309187 | 668 | 186 | 222 | 83 | e−117 | |
| 7455 | 2.305519035 | down | 0.0000387 | 0.000353675 | BI320411 | 278 | 51 | 76 | 67 | 2e−24 | protein amino acid phosphorylation |
| 7456 | 2.305448164 | down | 0.0000189 | 0.000215732 | BE475334 | 430 | 125 | 142 | 88 | 3e−71 | |
| 7457 | 2.305251704 | down | 0.0000229 | 0.000246966 | AI856374 | 834 | 94 | 135 | 69 | 3e−51 | signal transduction |
| 7458 | 2.304087148 | down | 0.0000233 | 0.000250375 | BU577239 | 594 | 122 | 183 | 66 | 1e−70 | DNA repair |
| 7459 | 2.30400862 | down | 0.0000125 | 0.00016339 | AW459223 | 340 | 14 | 43 | 32 | 0.85 | regulation of transcription\, DNA- |
| | | | | | | | | | | | dependent |
| 7460 | 2.303864773 | down | 0.0000113 | 0.000152786 | BQ079576 | 532 | 14 | 31 | 45 | 3.8 | sterol biosynthesis |
| 7461 | 2.303688433 | down | 0.0000125 | 0.000164022 | BE659092 | 589 | 24 | 70 | 34 | 2.7 | |
| 7462 | 2.303593222 | down | 0.000015 | 0.000185652 | BE659661 | 555 | 18 | 60 | 30 | 0.49 | |
| 7463 | 2.303401463 | down | 0.00038657 | 0.001823677 | AW309009 | 525 | 61 | 87 | 70 | 1e−30 | transport |
| 7464 | 2.302476194 | down | 0.000187264 | 0.001074892 | BI787881 | 505 | 122 | 162 | 75 | 2e−71 | protein amino acid phosphorylation |
| 7465 | 2.302184183 | down | 0.00000418 | 0.000081 | CA819040 | 472 | 45 | 89 | 50 | 8e−19 | |
| 7466 | 2.302092683 | down | 0.0000632 | 0.000498386 | AW351001 | 698 | 32 | 84 | 38 | 5e−15 | |
| 7467 | 2.301633048 | down | 0.0000531 | 0.000441119 | BF066728 | 344 | 105 | 114 | 92 | 3e−60 | proteolysis and peptidolysis |
| 7468 | 2.301307534 | down | 0.000137097 | 0.000861759 | BG362878 | 580 | 121 | 177 | 68 | 2e−73 | |
| 7469 | 2.301189804 | down | 0.000127261 | 0.000818678 | BM307513 | 343 | 17 | 27 | 62 | 0.004 | protein amino acid phosphorylation |
| 7470 | 2.301082787 | down | 0.0000612 | 0.000487012 | CA937565 | 408 | 17 | 42 | 40 | 0.97 | signal transduction |
| 7471 | 2.300578458 | down | 0.00000731 | 0.000114277 | L19360.1 | | | | | | protein kinase 2 |
| 7472 | 2.300535182 | down | 0.00000544 | 0.0000955 | CD390916 | 268 | 10 | 27 | 37 | 7.2 | |
| 7473 | 2.299975573 | down | 0.000588528 | 0.002479802 | AI443505 | 582 | 69 | 139 | 49 | 2e−30 | abscisic acid biosynthesis |
| 7474 | 2.299609042 | down | 0.000158227 | 0.00095347 | CF806746 | 725 | 150 | 183 | 81 | 8e−88 | |
| 7475 | 2.299486143 | down | 0.000194314 | 0.001103735 | AW782149 | 594 | 150 | 195 | 76 | 2e−84 | amino acid metabolism |
| 7476 | 2.298857524 | down | 0.0000348 | 0.000330641 | BQ613037 | 506 | 96 | 113 | 84 | 1e−48 | |
| 7477 | 2.298289728 | down | 0.00000796 | 0.000120296 | BU546482 | 522 | 15 | 35 | 42 | 2.8 | |
| 7478 | 2.297929152 | down | 0.00000548 | 0.000096 | CD417358 | 331 | 15 | 44 | 34 | 1.5 | |
| 7479 | 2.297335329 | down | 0.000016 | 0.000193231 | CD392732 | 601 | 91 | 128 | 71 | 5e−48 | protein amino acid phosphorylation |
| 7480 | 2.297228362 | down | 0.00000458 | 0.0000858 | CD410800 | 579 | 57 | 93 | 61 | 3e−27 | |
| 7481 | 2.296089934 | down | 0.00000513 | 0.0000925 | BU548185 | 271 | 66 | 88 | 75 | 1e−33 | protein amino acid phosphorylation |
| 7482 | 2.295410967 | down | 0.0000183 | 0.000211148 | BQ080878 | 582 | 79 | 134 | 58 | 4e−43 | metabolism |
| 7483 | 2.295404219 | down | 0.000026 | 0.00027023 | AI965636 | 489 | 79 | 141 | 56 | 7e−37 | potassium ion transport |
| 7484 | 2.295305871 | down | 0.0000441 | 0.000389141 | BM886509 | 569 | 58 | 77 | 75 | 1e−25 | ubiquitin-dependent protein catabolism |
| 7485 | 2.295278677 | down | 0.0000136 | 0.00017319 | BG511234 | 506 | 87 | 168 | 51 | 1e−42 | ubiquitin-dependent protein catabolism |
| 7486 | 2.295250193 | down | 0.000337902 | 0.001650201 | CF808990 | 705 | 165 | 222 | 74 | 2e−92 | Aspartate aminotransferase |
| | | | | | | | | | | | glyoxysomal isozyme AAT1 precursor |
| | | | | | | | | | | | and aspartate aminotransferase |
| | | | | | | | | | | | cytosolic isozyme AAT2 (AAT), leaf |
| | | | | | | | | | | | senescence |
| 7487 | 2.295173775 | down | 0.000205705 | 0.00115099 | CK606602 | 516 | 106 | 138 | 76 | 2e−59 | |
| 7488 | 2.295085209 | down | 0.00000496 | 0.0000905 | CD410230 | 527 | 11 | 21 | 52 | 6.4 | multidrug transport |
| 7489 | 2.29496408 | down | 0.0000064 | 0.000105663 | BM121610 | 748 | 42 | 70 | 60 | 7e−18 | lipid metabolism |
| 7490 | 2.294712428 | down | 0.0000304 | 0.000300825 | CA852256 | 651 | 63 | 105 | 60 | 5e−31 | protein amino acid phosphorylation |
| 7491 | 2.294680009 | down | 0.00000609 | 0.000102481 | BU081082 | 626 | 118 | 211 | 55 | 1e−73 | |
| 7492 | 2.294598437 | down | 0.000006 | 0.000101444 | CD409722 | 605 | 122 | 162 | 75 | 3e−71 | |
| 7493 | 2.294278479 | down | 0.000568227 | 0.002420807 | BI969561 | 776 | 86 | 124 | 69 | 8e−47 | transport |
| 7494 | 2.294202379 | down | 0.000545242 | 0.002344926 | BU090618 | 566 | 89 | 142 | 62 | 2e−38 | isoprenoid biosynthesis |
| 7495 | 2.293712783 | down | 0.0000761 | 0.000567269 | AW756085 | 561 | 62 | 132 | 46 | 2e−23 | |
| 7496 | 2.293669687 | down | 0.0000167 | 0.000198784 | CA801819 | 496 | 38 | 72 | 52 | 1e−12 | |
| 7497 | 2.292874404 | down | 0.000174443 | 0.001023438 | CD411438 | 608 | 106 | 115 | 92 | 1e−56 | protein biosynthesis |
| 7498 | 2.292815839 | down | 0.00000774 | 0.000118187 | BQ611893 | 291 | 31 | 39 | 79 | 2e−12 | |
| 7499 | 2.292632438 | down | 0.00000843 | 0.000124488 | CF807993 | 427 | 30 | 39 | 76 | 3e−12 | electron transport |
| 7500 | 2.292074196 | down | 0.001336293 | 0.004479796 | AW278033 | 631 | 146 | 203 | 71 | 4e−78 | branched chain family amino acid |
| | | | | | | | | | | | biosynthesis |
| 7501 | 2.291682044 | down | 0.000243913 | 0.001304869 | CD416961 | 673 | 43 | 62 | 69 | 8e−18 | protein biosynthesis |
| 7502 | 2.291562146 | down | 0.0000621 | 0.000492364 | CA783746 | 606 | 108 | 218 | 49 | 7e−52 | electron transport |
| 7503 | 2.291271949 | down | 0.0000157 | 0.000191459 | AI900498 | 453 | 98 | 151 | 64 | 4e−47 | regulation of transcription\, DNA- |
| | | | | | | | | | | | dependent |
| 7504 | 2.291232419 | down | 0.0000133 | 0.000170635 | AW733755 | 421 | 9 | 15 | 60 | 3.0 | amino acid transport |
| 7505 | 2.29120595 | down | 0.0000282 | 0.000285026 | CD394519 | 319 | 10 | 13 | 76 | 4.2 | |
| 7506 | 2.287650513 | down | 0.00063092 | 0.002605843 | BE820802 | 571 | 11 | 21 | 52 | 4.3 | protein amino acid phosphorylation |
| 7507 | 2.287458818 | down | 0.0000112 | 0.000151506 | AI496358 | 394 | 94 | 129 | 72 | 4e−50 | |
| 7508 | 2.287419794 | down | 0.000179335 | 0.001040329 | BI975097 | 568 | 143 | 166 | 86 | 4e−80 | selenocysteine incorporation |
| 7509 | 2.287113388 | down | 0.000194749 | 0.001105205 | CA800265 | 705 | 86 | 159 | 54 | 3e−41 | carbohydrate biosynthesis |
| 7510 | 2.286948495 | down | 0.000105785 | 0.000715135 | BU761957 | 534 | 124 | 162 | 76 | 1e−66 | protein amino acid phosphorylation |
| 7511 | 2.286718172 | down | 0.0000482 | 0.000413158 | AW310917 | 229 | 13 | 24 | 54 | 1.1 | |
| 7512 | 2.286711183 | down | 0.0000192 | 0.00021876 | BF424354 | 581 | 143 | 194 | 73 | 5e−79 | protein amino acid glycosylation |
| 7513 | 2.28655087 | down | 0.00000511 | 0.0000923 | BQ742835 | 425 | 41 | 104 | 39 | 1e−11 | tRNA aminoacylation for protein |
| | | | | | | | | | | | translation |
| 7514 | 2.286516875 | down | 0.0000349 | 0.000331401 | BE657930 | 422 | 28 | 47 | 59 | 9e−10 | intracellular protein transport |
| 7515 | 2.286472712 | down | 0.000170382 | 0.001006554 | BE823012 | 490 | 24 | 47 | 51 | 7e−08 | |
| 7516 | 2.286113284 | down | 0.000648127 | 0.002656782 | CF808862 | 681 | 95 | 120 | 79 | 1e−52 | pyridoxine biosynthesis |
| 7517 | 2.285911994 | down | 0.0000196 | 0.000221197 | AW830775 | 332 | 51 | 84 | 60 | 3e−26 | nitrogen metabolism |
| 7518 | 2.28494139 | down | 0.0000196 | 0.000221387 | BQ628915 | 214 | 13 | 33 | 39 | 7.2 | electron transport |
| 7519 | 2.284533578 | down | 0.0000874 | 0.000626939 | CA799718 | 714 | 64 | 228 | 28 | 5e−15 | protein amino acid phosphorylation |
| 7520 | 2.2844062 | down | 0.0000482 | 0.000413128 | CK605999 | 443 | 51 | 62 | 82 | 5e−23 | |
| 7521 | 2.283620076 | down | 0.00000524 | 0.0000934 | BM891745 | 569 | 124 | 197 | 62 | 3e−66 | auxin mediated signaling |
| 7522 | 2.283383139 | down | 0.0000574 | 0.000466469 | BI968921 | 781 | 155 | 233 | 66 | 8e−84 | DNA methylation |
| 7523 | 2.283074953 | down | 0.000734226 | 0.002902183 | CF806402 | 692 | 29 | 69 | 42 | 2e−07 | |
| 7524 | 2.283038975 | down | 0.000110246 | 0.00073721 | BG789742 | 434 | 83 | 144 | 57 | 3e−46 | carbohydrate metabolism |
| 7525 | 2.282925034 | down | 0.00000344 | 0.0000717 | CF807232 | 271 | 44 | 98 | 44 | 1e−12 | |
| 7526 | 2.282796177 | down | 0.00000802 | 0.000120883 | CD407497 | 569 | 75 | 105 | 71 | 9e−35 | |
| 7527 | 2.282641643 | down | 0.0000958 | 0.000669463 | AF301590.1 | | | | | | Cytosolic glutamine synthetase |
| | | | | | | | | | | | GSbeta1 |
| 7528 | 2.282484762 | down | 0.0000203 | 0.000226384 | CD392748 | 660 | 179 | 211 | 84 | e−104 | photosynthesis light harvesting |
| 7529 | 2.281789706 | down | 0.0000958 | 0.000669389 | AW234450 | 602 | 163 | 197 | 82 | 6e−98 | |
| 7530 | 2.281636615 | down | 0.0000106 | 0.000145879 | BG508767 | 211 | 11 | 28 | 39 | 3.3 | |
| 7531 | 2.281600676 | down | 0.0000134 | 0.000171238 | BE347874 | 619 | 89 | 155 | 57 | 4e−44 | |
| 7532 | 2.281370789 | down | 0.0000912 | 0.000646624 | CF808618 | 305 | 40 | 74 | 54 | 3e−14 | |
| 7533 | 2.281351867 | down | 0.0000039 | 0.0000775 | BU549173 | 574 | 36 | 83 | 43 | 2e−10 | |
| 7534 | 2.280717022 | down | 0.00003 | 0.000297721 | CD400282 | 496 | 52 | 68 | 76 | 2e−22 | |
| 7535 | 2.280189016 | down | 0.000197881 | 0.001118757 | BF424069 | 264 | 20 | 70 | 28 | 0.026 | apoptosis |
| 7536 | 2.280141371 | down | 0.0000174 | 0.000204776 | CD415693 | 595 | 30 | 36 | 83 | 8e−13 | |
| 7537 | 2.280036358 | down | 0.0000556 | 0.00045587 | BU550409 | 545 | 60 | 70 | 85 | 8e−30 | G-protein coupled receptor protein |
| | | | | | | | | | | | signaling pathway |
| 7538 | 2.279632378 | down | 0.000632048 | 0.002609931 | BE819881 | 516 | 71 | 109 | 65 | 9e−41 | cell wall organization and biogenesis |
| | | | | | | | | | | | (sensu Magnoliophyta) |
| 7539 | 2.279505904 | down | 0.0000078 | 0.000118925 | AW310721 | 679 | 109 | 143 | 76 | 5e−63 | |
| 7540 | 2.279160159 | down | 0.0000863 | 0.000620736 | BI973227 | 554 | 88 | 110 | 80 | 2e−43 | DNA methylation |
| 7541 | 2.278898001 | down | 0.00000676 | 0.000108826 | AJ239132.1 | | | | | | carbonic anhydrase |
| 7542 | 2.278608166 | down | 0.0000198 | 0.000222737 | BG237396 | 411 | 22 | 49 | 44 | 7e−04 | cellular morphogenesis during |
| | | | | | | | | | | | differentiation |
| 7543 | 2.27834341 | down | 0.0000103 | 0.000143348 | BU548348 | 645 | 103 | 122 | 84 | 7e−56 | |
| 7544 | 2.277791996 | down | 0.00000669 | 0.000108286 | BE556009 | 448 | 55 | 74 | 74 | 8e−24 | nucleosome assembly |
| 7545 | 2.277404881 | down | 0.0000149 | 0.000184746 | BG156309 | 487 | 115 | 161 | 71 | 7e−67 | |
| 7546 | 2.276214014 | down | 0.000344387 | 0.001672483 | AI494765 | 396 | 84 | 111 | 75 | 2e−44 | G-protein coupled receptor protein |
| | | | | | | | | | | | signaling pathway |
| 7547 | 2.27611116 | down | 0.00000655 | 0.000106681 | AW310285 | 641 | 140 | 177 | 79 | 5e−86 | |
| 7548 | 2.276066655 | down | 0.0000313 | 0.000307543 | BQ742012 | 571 | 95 | 204 | 46 | 3e−37 | |
| 7549 | 2.275850748 | down | 0.000139087 | 0.000870757 | BI699241 | 545 | 140 | 181 | 77 | 2e−78 | negative regulation of flower |
| | | | | | | | | | | | development |
| 7550 | 2.275722447 | down | 0.000172056 | 0.001013899 | AW350249 | 695 | 134 | 160 | 83 | 6e−77 | electron transport |
| 7551 | 2.275018391 | down | 0.0000186 | 0.000213297 | AI443601 | 374 | 24 | 46 | 52 | 7e−07 | regulation of cell cycle |
| 7552 | 2.274973447 | down | 0.00000739 | 0.0001153 | CD407026 | 370 | 17 | 48 | 35 | 2.2 | RNA processing |
| 7553 | 2.274659991 | down | 0.00057863 | 0.00245456 | CD393086 | 555 | 83 | 104 | 79 | 4e−43 | trehalose biosynthesis |
| 7554 | 2.274376316 | down | 0.0000337 | 0.000323272 | BM092475 | 420 | 17 | 68 | 25 | 6.6 | |
| 7555 | 2.274365125 | down | 0.00000314 | 0.0000678 | AW310673 | 434 | 45 | 86 | 52 | 5e−18 | |
| 7556 | 2.273714753 | down | 0.000144147 | 0.000892063 | BM177422 | 604 | 89 | 148 | 60 | 5e−45 | |
| 7557 | 2.273644465 | down | 0.000113093 | 0.000750342 | BE802373 | 653 | 149 | 225 | 66 | 7e−85 | |
| 7558 | 2.273568353 | down | 0.0000236 | 0.00025233 | BE821686 | 683 | 71 | 177 | 40 | 1e−28 | |
| 7559 | 2.272865264 | down | 0.000321944 | 0.001595467 | AB083027.1 | | | | | | syringolide-induced protein 1-3-1A |
| 7560 | 2.272305306 | down | 0.0000208 | 0.000230533 | CD416674 | 734 | 132 | 159 | 83 | 4e−73 | ribosomal protein S11 |
| 7561 | 2.272226573 | down | 0.0000115 | 0.000153886 | CF808936 | 552 | 118 | 167 | 70 | 6e−65 | metabolism |
| 7562 | 2.272072965 | down | 0.0000171 | 0.000201827 | AW706295 | 195 | 10 | 19 | 52 | 3.2 | |
| 7563 | 2.27119951 | down | 0.000058 | 0.000469661 | BI785350 | 579 | 117 | 152 | 76 | 9e−65 | |
| 7564 | 2.270400637 | down | 0.0000445 | 0.00039129 | CD398443 | 370 | 15 | 48 | 31 | 1.3 | |
| 7565 | 2.270318277 | down | 0.00000956 | 0.000135766 | BQ453323 | 434 | 9 | 27 | 33 | 1.1 | protein amino acid phosphorylation |
| 7566 | 2.269917833 | down | 0.001237882 | 0.004244983 | BE820284 | 740 | 73 | 180 | 40 | 8e−25 | |
| 7567 | 2.269875214 | down | 0.000009 | 0.000130277 | BU578170 | 547 | 57 | 136 | 41 | 6e−23 | |
| 7568 | 2.269720808 | down | 0.00000575 | 0.0000987 | BU083805 | 594 | 70 | 198 | 35 | 9e−31 | |
| 7569 | 2.269697222 | down | 0.000137723 | 0.000864541 | BE023596 | | | | | | |
| 7570 | 2.269456315 | down | 0.0000718 | 0.000544985 | BE658988 | 388 | 14 | 34 | 41 | 0.076 | |
| 7571 | 2.269245944 | down | 0.0000353 | 0.000333289 | AW733896 | 260 | 9 | 27 | 33 | 9.4 | |
| 7572 | 2.268836233 | down | 0.000161021 | 0.0009669 | BI699480 | 555 | 86 | 166 | 51 | 2e−39 | regulation of transcription |
| 7573 | 2.268617682 | down | 0.0000144 | 0.000180332 | BE058601 | 373 | 16 | 34 | 47 | 0.008 | |
| 7574 | 2.268427279 | down | 0.0000752 | 0.00056248 | BE191090 | 652 | 111 | 216 | 51 | 4e−57 | metabolism |
| 7575 | 2.268090914 | down | 0.000039 | 0.000355362 | BU548822 | 643 | 15 | 45 | 33 | 2.4 | protein catabolism |
| 7576 | 2.267808074 | down | 0.0000384 | 0.000352245 | BE821445 | 278 | 20 | 39 | 51 | 1e−05 | |
| 7577 | 2.267778898 | down | 0.000113128 | 0.000750342 | CF807237 | 668 | 151 | 174 | 86 | 2e−84 | protein biosynthesis |
| 7578 | 2.26740304 | down | 0.0000397 | 0.000359753 | BU081718 | 616 | 126 | 204 | 61 | 4e−68 | protein biosynthesis |
| 7579 | 2.267191527 | down | 0.00000518 | 0.0000929 | BU084372 | 420 | 63 | 77 | 81 | 1e−30 | protein biosynthesis |
| 7580 | 2.267041077 | down | 0.0000102 | 0.000142234 | AW101113 | 439 | 48 | 79 | 60 | 6e−21 | |
| 7581 | 2.26661826 | down | 0.00000948 | 0.00013499 | BI785280 | 577 | 38 | 77 | 49 | 9e−14 | |
| 7582 | 2.26625552 | down | 0.0000366 | 0.000341753 | BE823140 | 641 | 100 | 144 | 69 | 7e−56 | carbohydrate metabolism |
| 7583 | 2.266241282 | down | 0.000539617 | 0.002326588 | AW311417 | 674 | 174 | 213 | 81 | 1e−96 | |
| 7584 | 2.265635179 | down | 0.0000439 | 0.000387914 | BE659214 | 541 | 17 | 36 | 47 | 0.014 | |
| 7585 | 2.263569339 | down | 0.0000158 | 0.000192253 | CF808942 | 636 | 145 | 177 | 81 | 9e−80 | |
| 7586 | 2.263534483 | down | 0.0000127 | 0.000165597 | CA820282 | 598 | 115 | 186 | 61 | 3e−71 | |
| 7587 | 2.262037698 | down | 0.0000368 | 0.000342685 | BU764259 | 277 | 13 | 30 | 43 | 0.38 | |
| 7588 | 2.261965647 | down | 0.0000157 | 0.000191119 | BI971020 | 800 | 120 | 154 | 77 | 4e−72 | glycogen biosynthesis |
| 7589 | 2.261936983 | down | 0.000318062 | 0.001584369 | BE023382 | 456 | 123 | 151 | 81 | 1e−66 | protein amino acid phosphorylation |
| 7590 | 2.261933515 | down | 0.000451879 | 0.002041189 | BI426378 | 224 | 9 | 20 | 45 | 9.4 | cell wall biosynthesis (sensu |
| | | | | | | | | | | | Magnoliophyta) |
| 7591 | 2.261606522 | down | 0.000113165 | 0.000750459 | CF808984 | 652 | 38 | 79 | 48 | 6e−17 | protein amino acid phosphorylation |
| 7592 | 2.261574609 | down | 0.0000112 | 0.000151484 | CD418243 | 411 | 50 | 67 | 74 | 4e−26 | |
| 7593 | 2.26150006 | down | 0.000179549 | 0.00104101 | AW234995 | 583 | 169 | 192 | 88 | e−101 | carbohydrate metabolism |
| 7594 | 2.260653117 | down | 0.00000975 | 0.000137818 | BI427162 | 336 | 30 | 66 | 45 | 2e−08 | |
| 7595 | 2.260282159 | down | 0.000351467 | 0.001698358 | BE329712 | 461 | 109 | 150 | 72 | 3e−59 | glycogen biosynthesis |
| 7596 | 2.260198302 | down | 0.000006 | 0.000101462 | CD401345 | 477 | 34 | 46 | 73 | 1e−13 | nucleotide-sugar transport |
| 7597 | 2.259841807 | down | 0.0000485 | 0.000414391 | BU091712 | 382 | 16 | 48 | 33 | 0.009 | regulation of transcription\, DNA- |
| | | | | | | | | | | | dependent |
| 7598 | 2.259810274 | down | 0.000271473 | 0.001409219 | AI442629 | 543 | 117 | 158 | 74 | 3e−61 | |
| 7599 | 2.259762368 | down | 0.0000336 | 0.000323082 | CF807298 | 577 | 79 | 156 | 50 | 3e−38 | electron transport |
| 7600 | 2.259446576 | down | 0.0000138 | 0.00017522 | BE820520 | 375 | 19 | 26 | 73 | 4e−06 | regulation of transcription\, DNA- |
| | | | | | | | | | | | dependent |
| 7601 | 2.258933248 | down | 0.00000302 | 0.0000663 | BU965000 | 441 | 11 | 22 | 50 | 2.6 | |
| 7602 | 2.258925357 | down | 0.0000243 | 0.000257122 | BI967564 | 595 | 18 | 48 | 37 | 0.32 | |
| 7603 | 2.25880353 | down | 0.000200579 | 0.001130442 | CK606525 | 760 | 135 | 183 | 73 | 7e−75 | |
| 7604 | 2.258585548 | down | 0.0000326 | 0.000315717 | BQ079408 | 421 | 21 | 33 | 63 | 3e−07 | |
| 7605 | 2.258354059 | down | 0.0000126 | 0.000164679 | AI416868 | 486 | 71 | 84 | 84 | 9e−35 | |
| 7606 | 2.258217054 | down | 0.000421227 | 0.001940005 | BI892535 | 397 | 32 | 39 | 82 | 2e−14 | regulation of nitrogen utilization |
| 7607 | 2.258210397 | down | 0.0000782 | 0.000577719 | BE190254 | 416 | 50 | 62 | 80 | 4e−23 | |
| 7608 | 2.258200405 | down | 0.00000459 | 0.0000859 | CD404867 | 558 | 50 | 88 | 56 | 1e−20 | regulation of transcription |
| 7609 | 2.258198969 | down | 0.0000257 | 0.000267985 | BI787992 | 361 | 20 | 26 | 76 | 4e−06 | |
| 7610 | 2.257279113 | down | 0.00106133 | 0.003805973 | BM732315 | 554 | 81 | 160 | 50 | 3e−42 | response to brassinosteroid stimulus |
| 7611 | 2.25726439 | down | 0.0000168 | 0.000199552 | AW830151 | 429 | 89 | 109 | 81 | 4e−50 | metabolism |
| 7612 | 2.257029092 | down | 0.00000921 | 0.000132355 | BE440840 | 515 | 44 | 54 | 81 | 3e−18 | transport |
| 7613 | 2.256219519 | down | 0.0000536 | 0.000443561 | BU763411 | 577 | 142 | 192 | 73 | 3e−85 | metabolism |
| 7614 | 2.256161357 | down | 0.000153322 | 0.00093078 | BM094054 | 576 | 46 | 52 | 88 | 2e−22 | regulation of translation |
| 7615 | 2.256128953 | down | 0.000498177 | 0.002192913 | BE210777 | 473 | 30 | 57 | 52 | 8e−09 | |
| 7616 | 2.255867393 | down | 0.0000445 | 0.000391375 | AW311540 | 569 | 77 | 109 | 70 | 1e−41 | |
| 7617 | 2.255786605 | down | 0.0000596 | 0.000477552 | AI736149 | 321 | 11 | 18 | 61 | 4.1 | protein modification |
| 7618 | 2.255731259 | down | 0.0000772 | 0.000572346 | BI971984 | 553 | 138 | 184 | 75 | 2e−76 | |
| 7619 | 2.255401988 | down | 0.000178209 | 0.001037156 | AI442774 | 431 | 48 | 55 | 87 | 4e−24 | protein-O-fucosyltransferase 2 |
| 7620 | 2.255336532 | down | 0.0000278 | 0.000281906 | BI893997 | 516 | 28 | 61 | 45 | 3e−08 | |
| 7621 | 2.255205149 | down | 0.000113117 | 0.000750342 | BE347998 | 653 | 133 | 173 | 76 | 2e−77 | proteolysis and peptidolysis |
| 7622 | 2.255040713 | down | 0.00000307 | 0.0000669 | BE823860 | 517 | 28 | 38 | 73 | 3e−10 | proteolysis and peptidolysis |
| 7623 | 2.254787976 | down | 0.0000201 | 0.000224757 | CD398655 | 611 | 72 | 88 | 81 | 9e−39 | GMP biosynthesis |
| 7624 | 2.25441294 | down | 0.0000859 | 0.000618014 | BU548985 | 614 | 60 | 101 | 59 | 3e−28 | |
| 7625 | 2.254031958 | down | 0.000734483 | 0.002902786 | BQ741597 | 433 | 58 | 136 | 42 | 5e−15 | regulation of transcription\, DNA- |
| | | | | | | | | | | | dependent |
| 7626 | 2.253724552 | down | 0.000205856 | 0.001151387 | BM177863 | 499 | 14 | 33 | 42 | 0.68 | regulation of transcription\, DNA- |
| | | | | | | | | | | | dependent |
| 7627 | 2.253511227 | down | 0.0000102 | 0.000142234 | BM525482 | 485 | 39 | 45 | 86 | 1e−16 | |
| 7628 | 2.253399053 | down | 0.000151147 | 0.000921431 | AI939079 | | | | | | |
| 7629 | 2.252895038 | down | 0.000320366 | 0.001590882 | CF805719 | 504 | 150 | 167 | 89 | 4e−90 | cell wall biosynthesis (sensu |
| | | | | | | | | | | | Magnoliophyta) |
| 7630 | 2.252420429 | down | 0.0000978 | 0.000677928 | AW317180 | 495 | 12 | 30 | 40 | 1.5 | |
| 7631 | 2.252298515 | down | 0.000651456 | 0.002666653 | BI425255 | | | | | | |
| 7632 | 2.251894416 | down | 0.0000436 | 0.000386373 | AW394388 | 296 | 13 | 31 | 41 | 0.85 | |
| 7633 | 2.251824153 | down | 0.000146948 | 0.000903156 | BM308022 | 539 | 137 | 179 | 76 | 9e−79 | |
| 7634 | 2.251596985 | down | 0.0000235 | 0.000252144 | CA802782 | 655 | 42 | 98 | 42 | 2e−10 | |
| 7635 | 2.251430107 | down | 0.0000038 | 0.0000762 | CF806738 | 701 | 96 | 212 | 45 | 1e−47 | |
| 7636 | 2.251368656 | down | 0.00000887 | 0.000128818 | BI427232 | 555 | 103 | 181 | 56 | 2e−54 | protein amino acid phosphorylation |
| 7637 | 2.251082733 | down | 0.00000837 | 0.000124012 | CA935357 | 601 | 79 | 103 | 76 | 3e−45 | |
| 7638 | 2.251023509 | down | 0.002489704 | 0.006987593 | BI974876 | 555 | 46 | 153 | 30 | 4e−11 | |
| 7639 | 2.250560692 | down | 0.00000945 | 0.000134664 | BI967892 | 653 | 106 | 156 | 67 | 6e−57 | response to oxidative stress |
| 7640 | 2.250487838 | down | 0.000282587 | 0.001453073 | AW350695 | 493 | 8 | 24 | 33 | 9.5 | transcription initiation from Pol II |
| | | | | | | | | | | | promoter |
| 7641 | 2.25037681 | down | 0.000014 | 0.000176619 | BI316980 | 508 | 40 | 51 | 78 | 2e−16 | |
| 7642 | 2.250361706 | down | 0.000288111 | 0.001473838 | BU548935 | 554 | 30 | 66 | 45 | 1e−05 | |
| 7643 | 2.250041992 | down | 0.000405677 | 0.001888975 | CK605763 | 688 | 154 | 183 | 84 | 7e−89 | NADP-specific isocitrate dehydrogenase |
| | | | | | | | | | | | (idh1) mRNA, 3′ end., main pathways of |
| | | | | | | | | | | | carbohydrate metabolism |
| 7644 | 2.249989487 | down | 0.00000739 | 0.0001153 | CF807350 | 364 | 13 | 36 | 36 | 0.064 | carbohydrate metabolism |
| 7645 | 2.249982629 | down | 0.000138508 | 0.000868088 | BI426133 | 364 | 49 | 67 | 85 | 2e−23 | |
| 7646 | 2.249887181 | down | 0.0000972 | 0.000675908 | AW831457 | 242 | 58 | 66 | 87 | 7e−30 | |
| 7647 | 2.249713406 | down | 0.0000289 | 0.000289874 | BE820728 | | | | | | |
| 7648 | 2.249448022 | down | 0.001141968 | 0.004006582 | BQ079957 | 544 | 72 | 168 | 42 | 1e−30 | |
| 7649 | 2.249340722 | down | 0.0000382 | 0.000351887 | CA802734 | 350 | 15 | 23 | 65 | 2e−04 | |
| 7650 | 2.248822116 | down | 0.000141789 | 0.000882844 | AW348548 | 465 | 88 | 106 | 83 | 2e−46 | DNA replication |
| 7551 | 2.248668408 | down | 0.000174059 | 0.001022817 | AW395444 | 397 | 77 | 86 | 89 | 2e−41 | |
| 7652 | 2.248515732 | down | 0.0000291 | 0.000291143 | AW203611 | 591 | 100 | 154 | 64 | 4e−53 | protein amino acid dephosphorylation |
| 7653 | 2.248038096 | down | 0.001373518 | 0.004572799 | CA784580 | 681 | 104 | 120 | 86 | 2e−55 | galactose metabolism |
| 7654 | 2.247838744 | down | 0.0000197 | 0.000222117 | BM270192 | 317 | 13 | 29 | 44 | 2.4 | dicarboxylic acid transport |
| 7655 | 2.246857719 | down | 0.000227279 | 0.001236994 | BQ629324 | 385 | 20 | 41 | 48 | 3e−07 | |
| 7656 | 2.246815211 | down | 0.000431153 | 0.001975082 | BQ743039 | 405 | 41 | 47 | 87 | 4e−18 | protein biosynthesis |
| 7657 | 2.246029327 | down | 0.0000663 | 0.000515257 | CA783193 | 795 | 124 | 216 | 57 | 8e−71 | |
| 7658 | 2.245330614 | down | 0.0000343 | 0.000327554 | AW831584 | 572 | 102 | 154 | 66 | 5e−51 | |
| 7659 | 2.245073864 | down | 0.00000536 | 0.0000947 | CD412837 | 678 | 112 | 156 | 71 | 6e−67 | |
| 7660 | 2.244661099 | down | 0.0000114 | 0.000153845 | BI945652 | 239 | 8 | 24 | 33 | 5.4 | matrix metalloproteinase MMP2, |
| | | | | | | | | | | | metabolism |
| 7661 | 2.244497149 | down | 0.0000181 | 0.000209702 | AW350680 | 754 | 143 | 190 | 75 | 2e−76 | |
| 7662 | 2.244125341 | down | 0.0000279 | 0.000283265 | BM085101 | 464 | 61 | 68 | 89 | 3e−33 | electron transport |
| 7663 | 2.243864688 | down | 0.00000571 | 0.0000985 | CD414576 | 474 | 15 | 42 | 35 | 2.3 | |
| 7664 | 2.242859865 | down | 0.0000608 | 0.00048458 | BQ453652 | 438 | 52 | 63 | 82 | 9e−25 | |
| 7665 | 2.242721717 | down | 0.0000265 | 0.000273782 | CF808784 | 690 | 121 | 187 | 64 | 1e−65 | protein catabolism |
| 7666 | 2.242685837 | down | 0.0000131 | 0.000168723 | BE804769 | 522 | 68 | 121 | 56 | 6e−32 | |
| 7667 | 2.242600485 | down | 0.00033328 | 0.001634635 | BU547933 | 520 | 36 | 95 | 37 | 5e−12 | G-protein coupled receptor protein |
| | | | | | | | | | | | signaling pathway |
| 7668 | 2.242173517 | down | 0.000065 | 0.00050817 | CD406906 | 408 | 68 | 97 | 70 | 2e−34 | trichome branching (sensu |
| | | | | | | | | | | | Magnoliophyta) |
| 7669 | 2.241530437 | down | 0.001697224 | 0.005334618 | BI786637 | 503 | 20 | 51 | 39 | 2.0 | |
| 7670 | 2.24070922 | down | 0.0000154 | 0.000189375 | AW705251 | 553 | 128 | 163 | 78 | 3e−72 | protein amino acid phosphorylation |
| 7671 | 2.240643304 | down | 0.000390877 | 0.001838268 | BI427078 | 576 | 139 | 167 | 83 | 1e−75 | cysteine biosynthesis |
| 7672 | 2.240524053 | down | 0.0000107 | 0.000147294 | CF806379 | 479 | 63 | 73 | 86 | 2e−31 | transport |
| 7673 | 2.240208519 | down | 0.0000367 | 0.000341992 | CD391381 | 597 | 64 | 110 | 58 | 2e−26 | proteolysis and peptidolysis |
| 7674 | 2.239826303 | down | 0.00000622 | 0.000103975 | AW350237 | 605 | 26 | 74 | 35 | 0.023 | polysaccharide metabolism |
| 7675 | 2.239650497 | down | 0.0000215 | 0.000236118 | AW432463 | 313 | 11 | 25 | 44 | 1.9 | |
| 7676 | 2.239404521 | down | 0.000012 | 0.000158814 | BG154825 | 341 | 20 | 36 | 55 | 9e−06 | |
| 7677 | 2.239111946 | down | 0.000403644 | 0.001881371 | BE348127 | 492 | 122 | 164 | 74 | 5e−68 | amino acid metabolism |
| 7678 | 2.238996506 | down | 0.0000803 | 0.000589277 | BI426534 | 547 | 11 | 22 | 50 | 8.8 | |
| 7679 | 2.238955095 | down | 0.000820879 | 0.003154206 | CA801632 | 784 | 215 | 261 | 82 | e−118 | |
| 7680 | 2.23892934 | down | 0.000258885 | 0.001362678 | AW706620 | 337 | 54 | 68 | 79 | 1e−26 | signal transduction |
| 7681 | 2.238710965 | down | 0.000335054 | 0.001640671 | BE021632 | 479 | 70 | 158 | 44 | 8e−28 | electron transport |
| 7682 | 2.238492037 | down | 0.0000156 | 0.00019101 | CF808066 | 549 | 19 | 37 | 51 | 4e−04 | |
| 7683 | 2.238279825 | down | 0.0000287 | 0.000288411 | CD390893 | 555 | 18 | 31 | 58 | 0.004 | cell adhesion |
| 7684 | 2.23824371 | down | 0.00000371 | 0.0000751 | BU548284 | 688 | 17 | 43 | 39 | 3.5 | metabolism |
| 7685 | 2.237889049 | down | 0.0000562 | 0.000459972 | BI969660 | 671 | 79 | 111 | 71 | 3e−41 | methionine metabolism |
| 7686 | 2.237288756 | down | 0.0000114 | 0.000153674 | AW424122 | 457 | 31 | 113 | 27 | 1e−05 | |
| 7687 | 2.237224782 | down | 0.000141885 | 0.000883147 | BG508334 | 393 | 28 | 41 | 68 | 8e−10 | |
| 7688 | 2.236837523 | down | 0.00004 | 0.000361818 | BI967604 | 759 | 93 | 150 | 62 | 2e−48 | |
| 7689 | 2.236833682 | down | 0.0000336 | 0.000323082 | BU577987 | 539 | 14 | 27 | 51 | 3.0 | |
| 7690 | 2.23672364 | down | 0.0000118 | 0.000156315 | CF807708 | 735 | 147 | 243 | 60 | 4e−80 | proteolysis and peptidolysis |
| 7691 | 2.236666986 | down | 0.001237699 | 0.004244983 | BM086054 | 535 | 44 | 150 | 29 | 2e−07 | |
| 7692 | 2.236548913 | down | 0.000127881 | 0.000821259 | BQ610796 | 186 | 8 | 13 | 61 | 3.2 | response to UV |
| 7693 | 2.236256646 | down | 0.000179563 | 0.00104101 | CF808116 | 729 | 65 | 79 | 82 | 1e−31 | transport |
| 7694 | 2.236225471 | down | 0.000311451 | 0.001560531 | BF069083 | 434 | 40 | 52 | 76 | 5e−20 | proteolysis and peptidolysis |
| 7695 | 2.236079044 | down | 0.00011689 | 0.000768921 | BG154892 | 506 | 66 | 110 | 60 | 1e−33 | |
| 7696 | 2.235303064 | down | 0.0000399 | 0.000361147 | BI971033 | 796 | 122 | 188 | 64 | 2e−69 | transport |
| 7697 | 2.235241678 | down | 0.0000568 | 0.000463339 | AW349703 | 662 | 67 | 132 | 50 | 4e−28 | |
| 7698 | 2.234966011 | down | 0.0000471 | 0.000406647 | CK605851 | 294 | 22 | 60 | 36 | 0.009 | Lipoxygenase L-5 (vlxB) |
| 7699 | 2.234758981 | down | 0.0000118 | 0.000157062 | BI497844 | 335 | 18 | 30 | 60 | 4e−04 | |
| 7700 | 2.234754826 | down | 0.000174716 | 0.001024122 | BM307866 | 596 | 96 | 163 | 58 | 2e−51 | |
| 7701 | 2.234403566 | down | 0.000161381 | 0.000968286 | BI970450 | 781 | 69 | 141 | 48 | 8e−34 | |
| 7702 | 2.23432578 | down | 0.0000147 | 0.000183627 | CF808835 | 283 | 48 | 54 | 88 | 3e−25 | electron transport |
| 7703 | 2.234267036 | down | 0.0000368 | 0.000342685 | CD418465 | 714 | 79 | 159 | 49 | 1e−40 | electron transport |
| 7704 | 2.233687728 | down | 0.00000343 | 0.0000717 | CD413683 | 485 | 10 | 13 | 76 | 3.2 | |
| 7705 | 2.233528689 | down | 0.0000204 | 0.000227142 | BQ611262 | 393 | 14 | 14 | 100 | 0.061 | male meiotic spindle assembly (sensu |
| | | | | | | | | | | | Viridiplantae) |
| 7706 | 2.233405631 | down | 0.0000101 | 0.000141229 | AF098662.1 | | | | | | Clone GO2 unknown mRNA sequence |
| 7707 | 2.233319866 | down | 0.00000891 | 0.000129297 | BE022332 | 477 | 62 | 88 | 70 | 2e−35 | |
| 7708 | 2.233238738 | down | 0.0000263 | 0.000272167 | AW310578 | 262 | 11 | 19 | 57 | 0.29 | |
| 7709 | 2.232724716 | down | 0.00000634 | 0.000105028 | AW233990 | | | | | | |
| 7710 | 2.232688408 | down | 0.0000125 | 0.000163677 | BE058825 | 457 | 92 | 101 | 91 | 4e−47 | |
| 7711 | 2.232659846 | down | 0.0000145 | 0.000181653 | BG725747 | 361 | 17 | 39 | 43 | 3.4 | metabolism |
| 7712 | 2.232566582 | down | 0.000178375 | 0.001037805 | AW458020 | 455 | 71 | 83 | 85 | 4e−39 | regulation of meristem organization |
| 7713 | 2.232302758 | down | 0.0000825 | 0.000601261 | U43838.1 | | | | | | GmCK1p |
| 7714 | 2.232291806 | down | 0.0000338 | 0.000323921 | BU547024 | 677 | 41 | 73 | 56 | 5e−20 | protein amino acid phosphorylation |
| 7715 | 2.232238084 | down | 0.0000244 | 0.000258214 | CA783210 | 698 | 135 | 214 | 63 | 2e−78 | protein amino acid phosphorylation |
| 7716 | 2.232049079 | down | 0.000102474 | 0.000699412 | BU548402 | 603 | 90 | 125 | 72 | 4e−55 | |
| 7717 | 2.231645935 | down | 0.00000637 | 0.000105475 | AW705861 | 347 | 9 | 20 | 45 | 7.1 | proteolysis and peptidolysis |
| 7718 | 2.231526113 | down | 0.000175032 | 0.001025649 | BQ612124 | 568 | 58 | 100 | 58 | 2e−34 | proteolysis and peptidolysis |
| 7719 | 2.231503716 | down | 0.00000936 | 0.000133521 | BQ610726 | 607 | 121 | 179 | 67 | 3e−70 | proteolysis and peptidolysis |
| 7720 | 2.231473338 | down | 0.0000131 | 0.000169399 | BG046987 | 466 | 23 | 52 | 44 | 2e−05 | |
| 7721 | 2.231208875 | down | 0.0000943 | 0.000661013 | CD411822 | 610 | 76 | 99 | 76 | 3e−43 | sulfate assimilation |
| 7722 | 2.231164892 | down | 0.000062 | 0.000491486 | BU550215 | 622 | 100 | 142 | 70 | 1e−53 | jasmonic acid biosynthesis |
| 7723 | 2.230921303 | down | 0.0000667 | 0.000517819 | BU550157 | 648 | 34 | 122 | 27 | 3e−04 | |
| 7724 | 2.230627885 | down | 0.00000508 | 0.000092 | AW432620 | 375 | 30 | 56 | 53 | 8e−08 | |
| 7725 | 2.230103291 | down | 0.000269351 | 0.001400326 | BI967176 | 424 | 65 | 81 | 80 | 2e−35 | |
| 7726 | 2.229967019 | down | 0.0000072 | 0.000113242 | CD401243 | 554 | 46 | 63 | 73 | 1e−20 | |
| 7727 | 2.229698877 | down | 0.00004 | 0.000361818 | BU551319 | 584 | 87 | 180 | 48 | 5e−44 | protein amino acid phosphorylation |
| 7728 | 2.229454119 | down | 0.000162534 | 0.000972568 | BQ610556 | 330 | 45 | 53 | 84 | 5e−23 | pentose-phosphate shunl |
| 7729 | 2.229449677 | down | 0.0000102 | 0.000142234 | CF808975 | 550 | 171 | 183 | 93 | 2e−95 | acetyl-CoA biosynthesis |
| 7730 | 2.229416798 | down | 0.0000196 | 0.000221382 | CD404869 | 287 | 17 | 53 | 32 | 3.2 | |
| 7731 | 2.229197497 | down | 0.00000867 | 0.000126951 | BE657948 | 461 | 35 | 99 | 35 | 0.001 | regulation of transcription\, DNA- |
| | | | | | | | | | | | dependent |
| 7732 | 2.229089331 | down | 0.00000596 | 0.000100889 | BE823359 | 616 | 92 | 126 | 73 | 2e−54 | signal transduction |
| 7733 | 2.229037343 | down | 0.00000603 | 0.000101686 | CF807739 | 724 | 132 | 159 | 83 | 7e−73 | metabolism |
| 7734 | 2.228925377 | down | 0.000140665 | 0.000877006 | BI699754 | 479 | 12 | 27 | 44 | 1.8 | RNA dependent DNA replication |
| 7735 | 2.228830757 | down | 0.000597926 | 0.002504544 | AI900872 | 569 | 145 | 188 | 77 | 3e−82 | |
| 7736 | 2.228806837 | down | 0.0000613 | 0.000487695 | BI969945 | 767 | 63 | 107 | 58 | 8e−31 | D-ribose metabolism |
| 7737 | 2.22792648 | down | 0.000107669 | 0.000724481 | BI423744 | 547 | 98 | 187 | 52 | 1e−46 | |
| 7738 | 2.227834175 | down | 0.0000064 | 0.000105663 | BE440278 | 485 | 96 | 134 | 71 | 1e−51 | |
| 7739 | 2.227598372 | down | 0.0000244 | 0.000258214 | BQ629925 | 440 | 79 | 147 | 53 | 6e−42 | |
| 7740 | 2.226925485 | down | 0.00000988 | 0.000139176 | CA819005 | 554 | 109 | 183 | 59 | 5e−60 | regulation of transcription\, DNA- |
| | | | | | | | | | | | dependent |
| 7741 | 2.22686741 | down | 0.00000977 | 0.00013796 | BI970491 | 779 | 87 | 127 | 68 | 6e−49 | |
| 7742 | 2.226846153 | down | 0.00000771 | 0.000117799 | BI699590 | 572 | 49 | 104 | 47 | 1e−18 | |
| 7743 | 2.226596544 | down | 0.000537191 | 0.002319051 | BE806784 | 651 | 67 | 150 | 44 | 8e−33 | |
| 7744 | 2.226549718 | down | 0.000111234 | 0.000741642 | AI900723 | 564 | 112 | 185 | 60 | 3e−61 | proteolysis and peptidolysis |
| 7745 | 2.226096142 | down | 0.001029537 | 0.003723916 | BE610836 | 374 | 61 | 119 | 51 | 1e−32 | |
| 7746 | 2.225744489 | down | 0.0000977 | 0.000677863 | BI699189 | 306 | 16 | 47 | 34 | 0.22 | |
| 7747 | 2.225298012 | down | 0.000238848 | 0.001283657 | BI471609 | 497 | 66 | 79 | 83 | 4e−36 | |
| 7748 | 2.224541868 | down | 0.000120464 | 0.000787009 | CD405557 | 707 | 124 | 134 | 92 | 1e−68 | |
| 7749 | 2.224302283 | down | 0.0000836 | 0.000606003 | BU760883 | 451 | 47 | 74 | 63 | 7e−25 | |
| 7750 | 2.223532507 | down | 0.000398965 | 0.001864416 | AW348062 | 763 | 95 | 149 | 63 | 2e−53 | |
| 7751 | 2.223158392 | down | 0.000796876 | 0.003085618 | BI320279 | 436 | 65 | 132 | 49 | 7e−29 | |
| 7752 | 2.222835854 | down | 0.0000578 | 0.000468433 | BI320306 | 477 | 11 | 23 | 47 | 1.4 | |
| 7753 | 2.222732704 | down | 0.00045272 | 0.0020439 | BQ452979 | 422 | 81 | 140 | 57 | 6e−39 | transport |
| 7754 | 2.222730636 | down | 0.000249365 | 0.001325472 | BU760714 | 445 | 83 | 113 | 73 | 5e−39 | transport |
| 7755 | 2.222146206 | down | 0.000144601 | 0.000893549 | CD394786 | 247 | 13 | 44 | 29 | 3.2 | |
| 7756 | 2.221996896 | down | 0.000075 | 0.000561732 | CF806262 | 669 | 114 | 236 | 48 | 8e−47 | proteolysis and peptidolysis |
| 7757 | 2.221469287 | down | 0.000026 | 0.00027023 | BU547412 | 532 | 55 | 105 | 52 | 4e−25 | G-protein coupled receptor protein |
| | | | | | | | | | | | signaling pathway |
| 7758 | 2.221292827 | down | 0.0000166 | 0.000198349 | AW310771 | 268 | 8 | 25 | 32 | 4.2 | carbohydrate metabolism |
| 7759 | 2.220977496 | down | 0.0000786 | 0.000580402 | BU762486 | 389 | 20 | 65 | 30 | 0.39 | |
| 7760 | 2.220446147 | down | 0.0000209 | 0.000231381 | AW309814 | 677 | 60 | 131 | 45 | 2e−19 | |
| 7761 | 2.220322748 | down | 0.000430271 | 0.001971673 | CD403627 | 377 | 21 | 61 | 34 | 1.3 | regulation of transcription\, DNA- |
| | | | | | | | | | | | dependent |
| 7762 | 2.220148464 | down | 0.0000573 | 0.000466429 | CD415391 | 697 | 102 | 126 | 80 | 2e−50 | nucleosome assembly |
| 7763 | 2.219313087 | down | 0.0000687 | 0.000528425 | AW507595 | 446 | 42 | 86 | 48 | 1e−18 | |
| 7764 | 2.219035883 | down | 0.0000196 | 0.000221433 | BI973656 | 390 | 22 | 62 | 35 | 0.23 | |
| 7765 | 2.218946952 | down | 0.000680413 | 0.002749607 | CD403514 | 595 | 49 | 101 | 48 | 2e−22 | proton transport |
| 7766 | 2.218822138 | down | 0.0000464 | 0.000402814 | CA801727 | 634 | 95 | 179 | 53 | 2e−49 | |
| 7767 | 2.218810321 | down | 0.0000326 | 0.000315668 | AW459710 | 417 | 20 | 60 | 33 | 1.0 | |
| 7768 | 2.21878316 | down | 0.000770935 | 0.003012462 | AI794786 | 580 | 89 | 120 | 74 | 5e−52 | |
| 7769 | 2.218777817 | down | 0.000304383 | 0.001534727 | BE802851 | 467 | 75 | 85 | 88 | 9e−40 | |
| 7770 | 2.218609953 | down | 0.00000641 | 0.000105721 | CD403804 | 489 | 35 | 62 | 56 | 3e−13 | metabolism |
| 7771 | 2.217938996 | down | 0.0000983 | 0.000680225 | CD390358 | 634 | 133 | 169 | 78 | 2e−74 | |
| 7772 | 2.217631421 | down | 0.0000383 | 0.000352083 | AW351149 | 786 | 19 | 47 | 40 | 0.035 | |
| 7773 | 2.217220936 | down | 0.0000631 | 0.000497741 | CA801426 | 802 | 80 | 176 | 45 | 4e−34 | cell adhesion |
| 7774 | 2.217102601 | down | 0.0000183 | 0.000210869 | BI944375 | 578 | 52 | 90 | 57 | 6e−18 | ion transport |
| 7775 | 2.216111587 | down | 0.0000185 | 0.000212611 | AW317669 | 535 | 48 | 133 | 36 | 5e−10 | |
| 7776 | 2.215931353 | down | 0.000176477 | 0.001031225 | CA820600 | 586 | 80 | 102 | 78 | 5e−42 | |
| 7777 | 2.215752546 | down | 0.0000236 | 0.000252459 | BG882787 | 534 | 80 | 158 | 50 | 8e−32 | protein amino acid phosphorylation |
| 7778 | 2.215380697 | down | 0.000369462 | 0.001763505 | BG509311 | 525 | 129 | 175 | 73 | 1e−69 | carbohydrate metabolism |
| 7779 | 2.215109608 | down | 0.0000522 | 0.000436104 | AW458823 | 219 | 9 | 16 | 56 | 2.5 | proteolysis and peptidolysis |
| 7780 | 2.215031219 | down | 0.00000715 | 0.000112845 | BG154611 | | | | | | |
| 7781 | 2.214996197 | down | 0.000217224 | 0.001198221 | CA801303 | 765 | 121 | 248 | 48 | 6e−63 | |
| 7782 | 2.214944239 | down | 0.0000943 | 0.000660933 | CD402472 | 415 | 38 | 58 | 65 | 1e−16 | |
| 7783 | 2.213924872 | down | 0.000139939 | 0.00087325 | BU760976 | 444 | 87 | 134 | 64 | 7e−43 | protein kinase C activation |
| 7784 | 2.213813781 | down | 0.000679398 | 0.002747247 | AW507560 | 444 | 60 | 77 | 77 | 3e−29 | |
| 7785 | 2.21381242 | down | 0.0000224 | 0.000243053 | AW348865 | 769 | 163 | 206 | 79 | 9e−93 | deoxyribonucleoside diphosphate |
| | | | | | | | | | | | metabolism |
| 7786 | 2.213134565 | down | 0.0000144 | 0.000180668 | CA938233 | 560 | 20 | 29 | 68 | 7e−06 | |
| 7787 | 2.212530588 | down | 0.000180405 | 0.001044924 | BE352683 | 496 | 104 | 153 | 67 | 2e−55 | electron transport |
| 7788 | 2.212166049 | down | 0.0000178 | 0.000207329 | CF808876 | 548 | 98 | 171 | 57 | 2e−57 | |
| 7789 | 2.212157242 | down | 0.0000612 | 0.000487036 | BQ610748 | 421 | 49 | 73 | 67 | 1e−20 | glycolysis |
| 7790 | 2.21208077 | down | 0.00000952 | 0.000135414 | CD405423 | 579 | 66 | 137 | 48 | 6e−24 | |
| 7791 | 2.212003173 | down | 0.000026 | 0.00027023 | BE806525 | 507 | 26 | 96 | 27 | 0.31 | |
| 7792 | 2.211897038 | down | 0.0000106 | 0.000146441 | CD417450 | | | | | | |
| 7793 | 2.211691668 | down | 0.0000107 | 0.000146922 | AW348696 | 713 | 115 | 174 | 66 | 4e−60 | nucleotide-excision repair |
| 7794 | 2.211678255 | down | 0.0000104 | 0.000144328 | CD394548 | 701 | 87 | 175 | 49 | 8e−43 | regulation of transcription\, DNA- |
| | | | | | | | | | | | dependent |
| 7795 | 2.211590485 | down | 0.00000885 | 0.000128725 | BM093327 | 578 | 33 | 46 | 71 | 3e−12 | transport |
| 7796 | 2.211188341 | down | 0.0000503 | 0.000424667 | CA938468 | 585 | 37 | 92 | 40 | 2e−06 | |
| 7797 | 2.211167238 | down | 0.001239682 | 0.00424908 | BM893205 | 473 | 28 | 35 | 80 | 3e−12 | protein amino acid dephosphorylation |
| 7798 | 2.211058281 | down | 0.0000978 | 0.000677928 | BM521606 | 493 | 121 | 168 | 72 | 4e−64 | |
| 7799 | 2.21087044 | down | 0.000133902 | 0.000848258 | CD402535 | 721 | 141 | 167 | 84 | 3e−77 | |
| 7800 | 2.210832461 | down | 0.0000929 | 0.00065365 | BM522726 | 469 | 16 | 43 | 37 | 0.070 | proteolysis and peptidolysis |
| 7801 | 2.210542314 | down | 0.00000982 | 0.000138334 | CA802879 | 598 | 54 | 168 | 32 | 4e−12 | |
| 7802 | 2.210461559 | down | 0.000238135 | 0.001281068 | BI944625 | 500 | 104 | 167 | 62 | 3e−54 | glycolysis |
| 7803 | 2.210075651 | down | 0.0000785 | 0.0005797 | BU549977 | 665 | 104 | 175 | 59 | 9e−57 | regulation of transcription\, DNA- |
| | | | | | | | | | | | dependent |
| 7804 | 2.209946263 | down | 0.001114411 | 0.003940353 | CF806862 | 677 | 120 | 222 | 54 | 1e−63 | |
| 7805 | 2.209889542 | down | 0.0000609 | 0.000485397 | BG359305 | 322 | 67 | 107 | 62 | 5e−36 | antibiotic biosynthesis |
| 7806 | 2.209207265 | down | 0.000563701 | 0.002405339 | AI437736 | 537 | 107 | 179 | 59 | 2e−57 | galactose metabolism |
| 7807 | 2.20898691 | down | 0.0000366 | 0.000341807 | BQ611844 | 621 | 82 | 169 | 48 | 7e−39 | |
| 7808 | 2.208966144 | down | 0.0000671 | 0.000519503 | BI967635 | 226 | 12 | 27 | 44 | 3.2 | dTDP biosynthesis |
| 7809 | 2.208913546 | down | 0.000059 | 0.000475243 | BE210539 | 202 | 13 | 23 | 56 | 2.4 | transport |
| 7810 | 2.208758684 | down | 0.00000906 | 0.000130764 | AI443511 | 360 | 17 | 36 | 47 | 0.23 | regulation of transcription\, DNA- |
| | | | | | | | | | | | dependent |
| 7811 | 2.208650691 | down | 0.0000063 | 0.000104925 | BE822222 | 625 | 27 | 79 | 34 | 4e−05 | |
| 7812 | 2.208522732 | down | 0.000244769 | 0.001308203 | CD397063 | 624 | 58 | 77 | 75 | 2e−28 | |
| 7813 | 2.20826245 | down | 0.001563362 | 0.005026439 | BU927299 | 616 | 94 | 217 | 43 | 4e−38 | |
| 7814 | 2.208180193 | down | 0.0000396 | 0.000359015 | BG237749 | 378 | 11 | 25 | 44 | 3.9 | |
| 7815 | 2.208088382 | down | 0.0000082 | 0.000122359 | AW598082 | | | | | | |
| 7816 | 2.207633515 | down | 0.0000114 | 0.000153343 | BU550159 | 668 | 100 | 137 | 72 | 9e−57 | |
| 7817 | 2.207111143 | down | 0.000121768 | 0.000793414 | CF809296 | 336 | 38 | 67 | 56 | 8e−18 | electron transport |
| 7818 | 2.206991163 | down | 0.0000449 | 0.000393643 | AI960718 | 482 | 56 | 148 | 37 | 2e−16 | |
| 7819 | 2.206976065 | down | 0.000385071 | 0.001818235 | CA935400 | 360 | 55 | 71 | 77 | 1e−26 | transition metal ion homeostasis |
| 7820 | 2.206905352 | down | 0.0000631 | 0.000497577 | BU760812 | 637 | 28 | 34 | 82 | 9e−11 | |
| 7821 | 2.206882794 | down | 0.000116516 | 0.000767403 | BE022889 | 380 | 13 | 36 | 36 | 8.9 | |
| 7822 | 2.206738182 | down | 0.0000187 | 0.000213975 | AW706815 | 278 | 14 | 41 | 34 | 1.1 | |
| 7823 | 2.20601807 | down | 0.000162634 | 0.000973013 | BU547079 | 628 | 43 | 123 | 34 | 2e−12 | |
| 7824 | 2.205270608 | down | 0.0000956 | 0.000668711 | CA785282 | 435 | 80 | 110 | 72 | 6e−47 | |
| 7825 | 2.205022881 | down | 0.0000034 | 0.0000711 | AF243368.1 | | | | | | glutathione S-transferase GST 13 |
| 7826 | 2.204701451 | down | 0.0000101 | 0.000141195 | AW309684 | 585 | 85 | 105 | 80 | 1e−46 | |
| 7827 | 2.204618726 | down | 0.0000454 | 0.000397015 | AW317399 | 677 | 35 | 61 | 57 | 1e−14 | |
| 7828 | 2.20454954 | down | 0.000077 | 0.000571397 | BI893750 | 413 | 29 | 78 | 37 | 5e−05 | |
| 7829 | 2.204300927 | down | 0.000226629 | 0.001234532 | CF807077 | 471 | 58 | 90 | 64 | 4e−29 | |
| 7830 | 2.204133967 | down | 0.0000677 | 0.000522747 | CK605778 | 734 | 86 | 175 | 49 | 6e−43 | Vegetative storage protien mRNA |
| | | | | | | | | | | | (VSP27) |
| 7831 | 2.203667958 | down | 0.0000431 | 0.000382748 | CD398452 | 508 | 84 | 90 | 93 | 2e−44 | |
| 7832 | 2.203609279 | down | 0.0000754 | 0.000563373 | BU082677 | 563 | 65 | 134 | 48 | 6e−24 | |
| 7833 | 2.20344713 | down | 0.0000285 | 0.000287328 | BM954563 | 568 | 58 | 187 | 31 | 3e−18 | electron transport |
| 7834 | 2.203325959 | down | 0.0000652 | 0.000508891 | BE020345 | 435 | 133 | 145 | 91 | 4e−69 | DNA replication |
| 7835 | 2.202259923 | down | 0.000149081 | 0.000912979 | BQ740648 | 599 | 87 | 99 | 87 | 9e−49 | cell wall biosynthesis (sensu |
| | | | | | | | | | | | Magnoliophyta) |
| 7836 | 2.202178915 | down | 0.000136126 | 0.000857375 | BE821472 | 329 | 14 | 46 | 30 | 1.5 | DNA recombination |
| 7837 | 2.201874125 | down | 0.000644615 | 0.002646426 | AW780460 | 399 | 64 | 133 | 48 | 2e−29 | amino acid transport |
| 7838 | 2.201752273 | down | 0.00000498 | 0.0000907 | CD392589 | 584 | 46 | 96 | 47 | 7e−20 | |
| 7839 | 2.201525882 | down | 0.000143143 | 0.000888593 | BG157181 | 340 | 27 | 32 | 84 | 1e−08 | |
| 7840 | 2.201401199 | down | 0.0000279 | 0.000283265 | BI969862 | 797 | 140 | 224 | 62 | 1e−83 | carbohydrate metabolism |
| 7841 | 2.201156957 | down | 0.000463292 | 0.002078283 | CA820272 | 562 | 9 | 18 | 50 | 7.2 | |
| 7842 | 2.201103763 | down | 0.000791137 | 0.003069956 | BQ452751 | 383 | 17 | 19 | 89 | 0.007 | protein amino acid phosphorylation |
| 7843 | 2.201048312 | down | 0.0000433 | 0.000384615 | BM522842 | 279 | 16 | 46 | 34 | 0.49 | glycolysis |
| 7844 | 2.200901635 | down | 0.000806269 | 0.003114296 | AI522873 | 438 | 24 | 29 | 82 | 4e−09 | protein amino acid phosphorylation |
| 7845 | 2.200807809 | down | 0.0000373 | 0.000346182 | BQ742018 | 415 | 33 | 45 | 73 | 6e−15 | |
| 7846 | 2.200000143 | down | 0.0000598 | 0.000479229 | BG652106 | 518 | 82 | 91 | 90 | 1e−40 | nucleosome assembly |
| 7847 | 2.199885851 | down | 0.00033942 | 0.001655248 | BG359619 | 356 | 54 | 84 | 64 | 3e−28 | red\, far-red light phototransduction |
| 7848 | 2.199615459 | down | 0.0000137 | 0.000174178 | BU548031 | 595 | 95 | 108 | 87 | 1e−49 | |
| 7849 | 2.199522992 | down | 0.00000924 | 0.000132463 | CF808281 | 618 | 115 | 163 | 70 | 1e−57 | |
| 7850 | 2.199451931 | down | 0.0000156 | 0.00019101 | CF806895 | 504 | 140 | 164 | 85 | 6e−75 | two-component signal transduction |
| | | | | | | | | | | | system (phosphorelay) |
| 7851 | 2.19926494 | down | 0.0000666 | 0.00051734 | BQ133572 | 548 | 36 | 63 | 57 | 1e−09 | response to stress |
| 7852 | 2.198969336 | down | 0.0000116 | 0.00015508 | AW350347 | 771 | 113 | 147 | 76 | 8e−63 | L-serine biosynthesis |
| 7853 | 2.198876981 | down | 0.000146714 | 0.000902304 | BM270487 | 534 | 42 | 73 | 57 | 1e−19 | |
| 7854 | 2.198840245 | down | 0.0000155 | 0.000189652 | L01453.1 | | | | | | protein kinase |
| 7855 | 2.198710357 | down | 0.0000385 | 0.000352804 | BG509157 | 392 | 20 | 53 | 37 | 7.5 | |
| 7856 | 2.198500824 | down | 0.0000319 | 0.000310783 | BE803157 | 453 | 74 | 142 | 52 | 3e−31 | |
| 7857 | 2.198422254 | down | 0.0000203 | 0.000226572 | AW310095 | 648 | 87 | 144 | 60 | 6e−47 | |
| 7858 | 2.198412699 | down | 0.0000409 | 0.000368094 | CD415449 | 648 | 116 | 164 | 70 | 3e−66 | |
| 7859 | 2.198344599 | down | 0.00000338 | 0.0000709 | BQ611780 | 555 | 16 | 63 | 25 | 0.28 | fatty acid biosynthesis |
| 7860 | 2.198002998 | down | 0.000265936 | 0.001389293 | CA800337 | 789 | 215 | 261 | 82 | e−119 | nitrogen metabolism |
| 7861 | 2.19774074 | down | 0.0004722 | 0.002107918 | AW598659 | 550 | 80 | 104 | 76 | 2e−43 | proteolysis and peptidolysis |
| 7862 | 2.197192331 | down | 0.000277652 | 0.001432806 | AI442223 | 376 | 64 | 110 | 58 | 5e−30 | |
| 7863 | 2.196853673 | down | 0.0000354 | 0.000333289 | BI968224 | 464 | 15 | 56 | 26 | 3.8 | |
| 7864 | 2.196831468 | down | 0.0000369 | 0.000342814 | BM732811 | 576 | 81 | 192 | 42 | 4e−34 | |
| 7865 | 2.196796326 | down | 0.000177276 | 0.001034053 | BE658249 | 594 | 28 | 51 | 54 | 1e−06 | |
| 7866 | 2.196698148 | down | 0.000346208 | 0.001678993 | BU546557 | 668 | 13 | 32 | 40 | 7.4 | |
| 7867 | 2.196384223 | down | 0.0000081 | 0.000121516 | BM177567 | 418 | 10 | 25 | 40 | 1.3 | |
| 7868 | 2.196030884 | down | 0.0000495 | 0.000420618 | AW349258 | 679 | 19 | 48 | 39 | 0.69 | |
| 7869 | 2.195957959 | down | 0.0000141 | 0.000177842 | BG156754 | 176 | 9 | 22 | 40 | 5.5 | |
| 7870 | 2.195432217 | down | 0.000013 | 0.000168102 | CD399280 | 494 | 71 | 109 | 65 | 8e−37 | |
| 7871 | 2.195231417 | down | 0.0000198 | 0.000222658 | BE658958 | 218 | 10 | 16 | 62 | 9.4 | brassinosteroid biosynthesis |
| 7872 | 2.194991817 | down | 0.001804011 | 0.005561974 | CF808015 | 544 | 36 | 37 | 97 | 8e−17 | |
| 7873 | 2.194727949 | down | 0.000502593 | 0.002205451 | BM308613 | 609 | 114 | 141 | 80 | 7e−63 | protein amino acid phosphorylation |
| 7874 | 2.194282764 | down | 0.0000669 | 0.000518799 | BU083406 | 381 | 28 | 37 | 75 | 6e−09 | secretory pathway |
| 7875 | 2.193834876 | down | 0.0000198 | 0.000222823 | BG237980 | 677 | 106 | 171 | 61 | 2e−54 | |
| 7876 | 2.193509738 | down | 0.001106943 | 0.003924646 | AW704705 | 512 | 14 | 45 | 31 | 2.1 | electron transport |
| 7877 | 2.193399876 | down | 0.00000611 | 0.000102642 | CK605728 | 817 | 67 | 192 | 34 | 4e−28 | |
| 7878 | 2.193244898 | down | 0.0000125 | 0.00016339 | CF808180 | 713 | 143 | 171 | 83 | 3e−81 | Vacuolar processing enzyme 2 (VPE2), |
| | | | | | | | | | | | protein catabolism |
| 7879 | 2.192849391 | down | 0.0000507 | 0.000426999 | BI702134 | 246 | 55 | 81 | 67 | 3e−26 | |
| 7880 | 2.192541809 | down | 0.0000713 | 0.000542038 | CD394570 | 621 | 73 | 95 | 76 | 7e−40 | |
| 7881 | 2.192476277 | down | 0.000387366 | 0.001825269 | CD417934 | 520 | 63 | 91 | 69 | 5e−31 | protein biosynthesis |
| 7882 | 2.192139223 | down | 0.0000825 | 0.000601261 | CF805952 | 702 | 59 | 165 | 35 | 2e−16 | |
| 7883 | 2.192113441 | down | 0.000175405 | 0.001027196 | BU760895 | 472 | 23 | 47 | 48 | 3e−07 | transport |
| 7884 | 2.191876168 | down | 0.0000173 | 0.000203923 | BG237397 | | | | | | |
| 7885 | 2.19184412 | down | 0.0000722 | 0.000546574 | CD390621 | 337 | 15 | 22 | 68 | 0.035 | carbohydrate metabolism |
| 7886 | 2.191422204 | down | 0.0000541 | 0.000447122 | AW307476 | 546 | 115 | 180 | 63 | 3e−64 | |
| 7887 | 2.191213416 | down | 0.000195898 | 0.001109885 | BQ610956 | | | | | | |
| 7888 | 2.191176491 | down | 0.0000378 | 0.000348501 | AI496646 | 414 | 13 | 17 | 76 | 0.20 | |
| 7889 | 2.191118201 | down | 0.0000116 | 0.000155047 | AW831983 | 518 | 52 | 71 | 73 | 5e−27 | |
| 7890 | 2.191075457 | down | 0.000610378 | 0.002542216 | BQ628343 | 244 | 14 | 43 | 32 | 4.1 | |
| 7891 | 2.190917096 | down | 0.00000659 | 0.000107091 | BU544211 | 513 | 58 | 122 | 47 | 2e−24 | |
| 7892 | 2.190805348 | down | 0.000212016 | 0.001177437 | BF219563 | 676 | 27 | 60 | 45 | 9e−09 | regulation of transcription\, DNA- |
| | | | | | | | | | | | dependent |
| 7893 | 2.190672101 | down | 0.0000657 | 0.000511713 | BM890751 | 411 | 42 | 50 | 84 | 4e−19 | |
| 7894 | 2.190538117 | down | 0.00000908 | 0.000130994 | CF808180 | 713 | 143 | 171 | 83 | 3e−81 | Vacuolar processing enzyme 2 (VPE2), |
| | | | | | | | | | | | protein catabolism |
| 7895 | 2.190181396 | down | 0.00000891 | 0.000129297 | BG363156 | 535 | 87 | 141 | 61 | 2e−40 | |
| 7896 | 2.190120124 | down | 0.001325371 | 0.00445828 | BU577254 | 537 | 52 | 92 | 56 | 4e−26 | protein amino acid phosphorylation |
| 7897 | 2.190053751 | down | 0.0000165 | 0.000197577 | BI970266 | 752 | 86 | 135 | 63 | 4e−47 | biological_process unknown |
| 7898 | 2.189978809 | down | 0.0000214 | 0.000234853 | BQ742425 | 539 | 29 | 46 | 63 | 3e−14 | regulation of transcription\, DNA- |
| | | | | | | | | | | | dependent |
| 7899 | 2.189460177 | down | 0.00000371 | 0.0000752 | CA851709 | 455 | 14 | 23 | 60 | 0.006 | proteolysis and peptidolysis |
| 7900 | 2.189391742 | down | 0.00000754 | 0.000116381 | CD403765 | 614 | 37 | 53 | 69 | 1e−11 | |
| 7901 | 2.189297042 | down | 0.000330961 | 0.001626654 | AW309187 | 668 | 186 | 222 | 83 | e−117 | |
| 7902 | 2.188859276 | down | 0.001036635 | 0.003142263 | CF808827 | 577 | 131 | 134 | 97 | 5e−73 | |
| 7903 | 2.188743427 | down | 0.0000319 | 0.000310783 | CD392405 | 657 | 125 | 149 | 83 | 1e−70 | carbohydrate metabolism |
| 7904 | 2.188712588 | down | 0.000740625 | 0.002921119 | CD412288 | 610 | 57 | 108 | 52 | 5e−31 | |
| 7905 | 2.188535931 | down | 0.00000618 | 0.000103437 | CA800500 | 698 | 31 | 93 | 33 | 1e−08 | |
| 7906 | 2.188519217 | down | 0.0000833 | 0.000604355 | BU761530 | 610 | 70 | 132 | 53 | 3e−38 | |
| 7907 | 2.18847863 | down | 0.0000121 | 0.000160042 | AI736209 | 444 | 32 | 57 | 56 | 6e−11 | |
| 7908 | 2.18829466 | down | 0.0000328 | 0.000317061 | BE822117 | 613 | 12 | 52 | 23 | 1.7 | regulation of global transcription from |
| | | | | | | | | | | | Pol II promoter |
| 7909 | 2.188091266 | down | 0.0000458 | 0.000399128 | BI967245 | 774 | 119 | 147 | 80 | 1e−66 | protein amino acid phosphorylation |
| 7910 | 2.187614719 | down | 0.000042 | 0.000375705 | BQ613096 | 552 | 19 | 58 | 32 | 1.8 | carbohydrate metabolism |
| 7911 | 2.187208749 | down | 0.000612519 | 0.002548594 | CA953389 | 574 | 96 | 150 | 64 | 8e−50 | protein amino acid phosphorylation |
| 7912 | 2.186957884 | down | 0.0000127 | 0.000165664 | BG239480 | 418 | 98 | 139 | 70 | 3e−52 | |
| 7913 | 2.186824399 | down | 0.000688803 | 0.002772549 | CF806744 | 535 | 109 | 149 | 73 | 5e−62 | |
| 7914 | 2.186402775 | down | 0.0000162 | 0.000195638 | CD392911 | 429 | 26 | 91 | 28 | 0.64 | proteolysis and peptidolysis |
| 7915 | 2.186082058 | down | 0.0000183 | 0.000210717 | CD411729 | 296 | 14 | 35 | 40 | 3.2 | |
| 7916 | 2.185895294 | down | 0.000492494 | 0.002174413 | CF807162 | 381 | 10 | 13 | 76 | 8.9 | |
| 7917 | 2.185568415 | down | 0.00003 | 0.000297921 | CD391951 | 653 | 33 | 71 | 46 | 8e−10 | |
| 7918 | 2.185553663 | down | 0.0000216 | 0.000236357 | CD408212 | 631 | 16 | 23 | 69 | 0.002 | |
| 7919 | 2.185196733 | down | 0.0000916 | 0.000647935 | AW348511 | 738 | 116 | 184 | 63 | 1e−64 | |
| 7920 | 2.185012304 | down | 0.000674379 | 0.002732933 | BG238475 | 646 | 80 | 99 | 80 | 3e−45 | |
| 7921 | 2.184982488 | down | 0.00019199 | 0.001093672 | BU545032 | 642 | 30 | 74 | 40 | 2e−10 | |
| 7922 | 2.184612722 | down | 0.0000363 | 0.000339542 | BG363173 | 479 | 35 | 66 | 53 | 4e−10 | |
| 7923 | 2.18454431 | down | 0.00000767 | 0.000117626 | CD410619 | 640 | 90 | 130 | 69 | 3e−46 | |
| 7924 | 2.184434821 | down | 0.0000148 | 0.000183966 | CD394694 | 595 | 45 | 61 | 73 | 1e−17 | |
| 7925 | 2.18441188 | down | 0.00029597 | 0.001504 | BE821735 | 722 | 36 | 101 | 35 | 3e−04 | regulation of transcription |
| 7926 | 2.184368628 | down | 0.0000255 | 0.000266509 | BU551006 | 698 | 63 | 102 | 61 | 6e−29 | |
| 7927 | 2.184199204 | down | 0000052 | 0.000434322 | BI971504 | 784 | 73 | 149 | 48 | 3e−28 | |
| 7928 | 2.184165917 | down | 0.0000264 | 0.000272707 | BG507880 | 527 | 73 | 177 | 41 | 4e−34 | protein amino acid phosphorylation |
| 7929 | 2.183906459 | down | 0.000396577 | 0.001857638 | BF595478 | 580 | 81 | 176 | 46 | 2e−39 | |
| 7930 | 2.183905308 | down | 0.0000665 | 0.000517046 | CA801713 | 635 | 172 | 210 | 81 | e−101 | ‘de novo’ pyrimidine base biosynthesis |
| 7931 | 2.183668104 | down | 0.00000929 | 0.000132818 | AW310458 | 649 | 79 | 165 | 47 | 3e−36 | protein amino acid phosphorylation |
| 7932 | 2.18324418 | down | 0.0000082 | 0.000122359 | AW309836 | 687 | 63 | 153 | 41 | 3e−24 | |
| 7933 | 2.18314451 | down | 0.0000261 | 0.000270651 | BM886509 | 569 | 58 | 77 | 75 | 1e−25 | ubiquitin-dependent protein catabolism |
| 7934 | 2.183046254 | down | 0.0000172 | 0.000203268 | BM732369 | 564 | 33 | 84 | 39 | 6e−16 | |
| 7935 | 2.18243508 | down | 0.0000157 | 0.00019101 | BU550316 | 505 | 28 | 35 | 80 | 1e−12 | carbohydrate metabolism |
| 7936 | 2.165973221 | down | 0.000108646 | 0.000729228 | BQ473937 | 579 | 95 | 116 | 81 | 2e−46 | protein amino acid phosphorylation |
| 7937 | 2.165892839 | down | 0.000071 | 0.000541154 | CF806264 | 237 | 11 | 18 | 61 | 2.4 | regulation of transcription\, DNA- |
| | | | | | | | | | | | dependent |
| 7938 | 2.165683658 | down | 0.00008 | 0.000588138 | BE020159 | 577 | 138 | 193 | 71 | 4e−84 | |
| 7939 | 2.165301748 | down | 0.00000515 | 0.0000928 | BQ081062 | 420 | 46 | 88 | 52 | 1e−20 | |
| 7940 | 2.164885755 | down | 0.00000587 | 0.000100013 | CA819652 | 243 | 20 | 60 | 33 | 0.49 | cell wall catabolism |
| 7941 | 2.164810065 | down | 0.0000788 | 0.000580732 | CD418111 | 669 | 39 | 102 | 38 | 4e−08 | |
| 7942 | 2.164808948 | down | 0.001020676 | 0.003700049 | BE554943 | 477 | 26 | 64 | 40 | 0.002 | regulation of transcription\, DNA- |
| | | | | | | | | | | | dependent |
| 7943 | 2.164079208 | down | 0.000131109 | 0.000836138 | BU549072 | 379 | 11 | 32 | 34 | 0.27 | |
| 7944 | 2.163933381 | down | 0.0000287 | 0.00028877 | CD398574 | 441 | 24 | 55 | 43 | 2e−09 | carbohydrate metabolism |
| 7945 | 2.163148913 | down | 0.000360922 | 0.00173204 | AI460917 | 472 | 14 | 45 | 31 | 0.055 | signal transduction |
| 7946 | 2.163135442 | down | 0.0000821 | 0.000599045 | BE803848 | 635 | 74 | 165 | 44 | 3e−31 | |
| 7947 | 2.163129488 | down | 0.0000374 | 0.000346275 | CD399383 | 640 | 85 | 86 | 98 | 1e−41 | nucleosome assembly |
| 7948 | 2.163118227 | down | 0.0000163 | 0.000196399 | BE058566 | 309 | 12 | 37 | 32 | 3.2 | aromatic amino acid family metabolism |
| 7949 | 2.163067013 | down | 0.0000184 | 0.00021144 | BM092794 | 365 | 21 | 36 | 58 | 7e−04 | |
| 7950 | 2.162961455 | down | 0.0000201 | 0.000224309 | CD401772 | 630 | 44 | 73 | 60 | 5e−19 | |
| 7951 | 2.162795606 | down | 0.0000494 | 0.0004195 | AI960688 | 484 | 101 | 162 | 62 | 1e−52 | |
| 7952 | 2.162742226 | down | 0.000347675 | 0.001684804 | CF805768 | 552 | 99 | 115 | 86 | 3e−56 | electron transport |
| 7953 | 2.162661565 | down | 0.0000288 | 0.000288977 | BE657461 | 733 | 43 | 137 | 31 | 4e−11 | dicarboxylic acid transport |
| 7954 | 2.162326564 | down | 0.0000485 | 0.000414391 | CF807781 | 169 | 16 | 27 | 59 | 0.86 | brassinosteroid mediated signaling |
| 7955 | 2.162261115 | down | 0.0000812 | 0.000594362 | BU545788 | 272 | 22 | 37 | 59 | 4e−09 | electron transport |
| 7956 | 2.162206268 | down | 0.0000633 | 0.000498697 | BQ298550 | 448 | 32 | 60 | 53 | 4e−11 | electron transport |
| 7957 | 2.161930551 | down | 0.0000146 | 0.000181757 | BI969642 | 784 | 72 | 149 | 48 | 2e−33 | protein amino acid phosphorylation |
| 7958 | 2.161905052 | down | 0.000750046 | 0.00294922 | AW317392 | 610 | 155 | 201 | 77 | 2e−94 | steroid metabolism |
| 7959 | 2.161730905 | down | 0.000194456 | 0.001104031 | BE805343 | 531 | 120 | 177 | 67 | 2e−71 | |
| 7960 | 2.16168963 | down | 0.0000209 | 0.000230993 | CD396853 | 700 | 45 | 100 | 45 | 2e−17 | negative regulation of flower |
| | | | | | | | | | | | development |
| 7961 | 2.161667454 | down | 0.0000262 | 0.000271615 | CF809102 | 663 | 106 | 193 | 54 | 5e−60 | |
| 7962 | 2.161660255 | down | 0.000119426 | 0.000781896 | BE806787 | 500 | 90 | 166 | 54 | 2e−48 | |
| 7963 | 2.161532179 | down | 0.00000572 | 0.0000985 | BI974203 | 585 | 61 | 136 | 44 | 6e−27 | |
| 7964 | 2.1612448 | down | 0.001146278 | 0.004013786 | AW569086 | 525 | 117 | 143 | 81 | 3e−69 | cationic peroxidase 2 /// peroxidase |
| 7965 | 2.16118053 | down | 0.000462967 | 0.002078048 | BU927486 | 586 | 42 | 134 | 31 | 7e−09 | carbohydrate metabolism |
| 7966 | 2.161069032 | down | 0.001123027 | 0.003962991 | BG042411 | 407 | 65 | 87 | 74 | 8e−32 | |
| 7967 | 2.161066278 | down | 0.000134197 | 0.000849473 | CA819047 | 270 | 10 | 37 | 27 | 0.84 | glycerol-3-phosphate metabolism |
| 7968 | 2.160689465 | down | 0.000119444 | 0.000781896 | CF807474 | 433 | 26 | 42 | 61 | 3e−10 | |
| 7969 | 2.160201319 | down | 0.0000448 | 0.000393074 | AI416848 | 412 | 42 | 56 | 75 | 4e−19 | |
| 7970 | 2.160164275 | down | 0.0000317 | 0.000309786 | BG046544 | 504 | 21 | 67 | 31 | 0.91 | regulation of transcription\, DNA- |
| | | | | | | | | | | | dependent |
| 7971 | 2.120488936 | down | 0.0000682 | 0.000525366 | BI699442 | 540 | 64 | 187 | 34 | 5e−19 | protein folding |
| 7972 | 2.120310607 | down | 0.0000276 | 0.000280947 | CF809032 | 660 | 138 | 220 | 62 | 3e−70 | metabolism |
| 7973 | 2.120225497 | down | 0.0000319 | 0.00031075 | BU547775 | 634 | 96 | 111 | 86 | 6e−49 | |
| 7974 | 2.119500927 | down | 0.0000868 | 0.000623781 | AW306221 | 609 | 60 | 86 | 69 | 2e−29 | |
| 7975 | 2.11928212 | down | 0.000156693 | 0.000946606 | BG508005 | 496 | 53 | 166 | 31 | 6e−12 | protein amino acid phosphorylation |
| 7976 | 2.119007691 | down | 0.000577249 | 0.002450083 | BE346184 | 617 | 158 | 205 | 77 | 3e−97 | carbohydrate metabolism |
| 7977 | 2.118709519 | down | 0.0000215 | 0.000236118 | BQ610800 | 552 | 66 | 90 | 73 | 7e−34 | electron transport |
| 7978 | 2.118684556 | down | 0.0000114 | 0.000153515 | BI968925 | 800 | 16 | 46 | 34 | 0.002 | ER to Golgi transport |
| 7979 | 2.118506348 | down | 0.0000627 | 0.000495471 | CF805655 | 501 | 65 | 69 | 94 | 2e−32 | protein biosynthesis |
| 7980 | 2.118344409 | down | 0.000369378 | 0.001763505 | BI785447 | 562 | 42 | 87 | 48 | 2e−11 | |
| 7981 | 2.118279517 | down | 0.0000156 | 0.000190362 | CD410224 | 616 | 73 | 109 | 66 | 3e−41 | electron transport |
| 7982 | 2.118192832 | down | 0.0000171 | 0.00020248 | BE824174 | 785 | 17 | 32 | 53 | 2e−05 | |
| 7983 | 2.118165863 | down | 0.0000496 | 0.000420793 | CD396224 | 611 | 44 | 59 | 74 | 8e−19 | |
| 7984 | 2.118110892 | down | 0.0000324 | 0.000314624 | AW203826 | 426 | 79 | 147 | 53 | 1e−35 | |
| 7985 | 2.117961124 | down | 0.0000185 | 0.000212611 | AW317166 | 646 | 56 | 100 | 56 | 3e−29 | protein amino acid phosphorylation |
| 7986 | 2.117923541 | down | 0.000247933 | 0.001320554 | CD392619 | 640 | 59 | 82 | 71 | 1e−30 | electron transport |
| 7987 | 2.117316775 | down | 0.000133087 | 0.000845024 | CA803163 | 444 | 33 | 37 | 89 | 2e−16 | carbohydrate biosynthesis |
| 7988 | 2.117257498 | down | 0.000204392 | 0.001146089 | BI699179 | 370 | 68 | 96 | 70 | 5e−36 | proteolysis and peptidolysis |
| 7989 | 2.116889974 | down | 0.000188764 | 0.00108004 | BE211206 | 577 | 96 | 192 | 50 | 7e−41 | |
| 7990 | 2.074239888 | down | 0.0000112 | 0.000151484 | BG653673 | 463 | 27 | 31 | 87 | 4e−10 | |
| 7991 | 2.074236822 | down | 0.0000616 | 0.00048928 | CA819511 | 343 | 17 | 47 | 36 | 4.2 | |
| 7992 | 2.073829444 | down | 0.0000439 | 0.000387914 | CD404676 | 719 | 89 | 119 | 74 | 3e−42 | nucleosome assembly |
| 7993 | 2.073741296 | down | 0.0002781 | 0.001434314 | BI971954 | 550 | 139 | 181 | 76 | 2e−78 | glycolysis |
| 7994 | 2.073582332 | down | 0.0000219 | 0.000238842 | AW102270 | 298 | 10 | 26 | 38 | 1.5 | |
| 7995 | 2.073194247 | down | 0.000157001 | 0.000947905 | CD410111 | | | | | | |
| 7996 | 2.072987147 | down | 0.000319604 | 0.00158887 | CF807310 | 681 | 82 | 185 | 44 | 3e−33 | |
| 7997 | 2.072911497 | down | 0.0000253 | 0.0002648 | AW101113 | 439 | 48 | 79 | 60 | 6e−21 | |
| 7998 | 2.072856903 | down | 0.000764493 | 0.002991511 | AI440627 | 446 | 109 | 131 | 83 | 6e−61 | protein catabolism |
| 7999 | 2.072781855 | down | 0.0000112 | 0.000151484 | AI938021 | 501 | 16 | 43 | 37 | 2.0 | |
| 8000 | 2.072710094 | down | 0.000018 | 0.000208796 | CD407758 | 590 | 136 | 165 | 82 | 9e−86 | carbohydrate biosynthesis |
| 8001 | 2.072357007 | down | 0.0000621 | 0.000491902 | BG352685 | 468 | 120 | 160 | 75 | 1e−66 | |
| 8002 | 2.07216448 | down | 0.000152856 | 0.000928839 | CA801912 | 567 | 63 | 116 | 54 | 3e−26 | G1 phase of mitotic cell cycle |
| 8003 | 2.072004729 | down | 0.000023 | 0.000247958 | BE657901 | | | | | | |
| 8004 | 2.071764969 | down | 0.001026646 | 0.003714529 | CD416427 | 616 | 77 | 115 | 66 | 5e−37 | amino acid transport |
| 8005 | 2.182325914 | down | 0.0000125 | 0.000163747 | BU551196 | 467 | 100 | 132 | 75 | 2e−55 | |
| 8006 | 2.181940463 | down | 0.0000117 | 0.000155589 | BE610251 | 513 | 123 | 172 | 71 | 4e−69 | metabolism |
| 8007 | 2.181917795 | down | 0.0000441 | 0.000389174 | BU550808 | 576 | 50 | 95 | 52 | 1e−23 | |
| 8008 | 2.181700537 | down | 0.0000877 | 0.000628608 | AW597528 | 476 | 29 | 34 | 85 | 2e−12 | cellular morphogenesis |
| 8009 | 2.18148093 | down | 0.000128518 | 0.000824225 | BI972497 | 560 | 154 | 185 | 83 | 1e−87 | transport |
| 8010 | 2.181276493 | down | 0.0000878 | 0.000628608 | BU761717 | 512 | 35 | 71 | 49 | 7e−14 | |
| 8011 | 2.181257587 | down | 0.0000271 | 0.00027733 | BE659549 | 773 | 193 | 227 | 85 | e−108 | L-phenylalanine biosynthesis |
| 8012 | 2.180875223 | down | 0.0000361 | 0.000338456 | AW318037 | 685 | 141 | 215 | 65 | 2e−78 | aromatic compound metabolism |
| 8013 | 2.180760166 | down | 0.0000063 | 0.000104931 | CA851929 | 600 | 17 | 48 | 35 | 0.43 | protein amino acid phosphorylation |
| 8014 | 2.180523687 | down | 0.0000436 | 0.000386352 | BM093461 | 542 | 13 | 34 | 38 | 2.3 | chemotaxis |
| 8015 | 2.180498881 | down | 0.0000241 | 0.000256213 | CD392167 | 634 | 48 | 107 | 44 | 8e−20 | |
| 8016 | 2.180353012 | down | 0.0000511 | 0.000429453 | AW277368 | 457 | 71 | 129 | 55 | 6e−40 | |
| 8017 | 2.180296634 | down | 0.0000222 | 0.000241338 | BU764579 | 421 | 78 | 96 | 81 | 1e−41 | |
| 8018 | 2.179960094 | down | 0.00000504 | 0.0000914 | BF010117 | 320 | 21 | 85 | 24 | 0.22 | |
| 8019 | 2.179770212 | down | 0.000026 | 0.00027023 | CD402926 | 629 | 73 | 94 | 77 | 2e−36 | fatty acid biosynthesis |
| 8020 | 2.179707151 | down | 0.0000211 | 0.000232924 | BE821279 | 662 | 87 | 152 | 57 | 2e−46 | |
| 8021 | 2.17950983 | down | 0.0000446 | 0.000391609 | AW703774 | 579 | 111 | 152 | 73 | 3e−64 | lipid catabolism |
| 8022 | 2.179173407 | down | 0.00022931 | 0.001245524 | BF598957 | 446 | 17 | 38 | 44 | 0.31 | |
| 8023 | 2.179047237 | down | 0.0000473 | 0.000408196 | CF807561 | 576 | 23 | 84 | 27 | 3e−09 | 2-hydroxyisoflavanone dehydratase |
| 8024 | 2.178765097 | down | 0.00011888 | 0.000778821 | CD400364 | 489 | 59 | 95 | 62 | 1e−28 | cysteine proteinase inhibitor |
| 8025 | 2.178727581 | down | 0.00000764 | 0.000117279 | CK605927 | 442 | 12 | 38 | 31 | 3.4 | Hydroxyproline-rich glycoprotein (hrgp), |
| | | | | | | | | | | | cell growth and/or maintenance |
| 8026 | 2.178718193 | down | 0.00000439 | 0.0000836 | BU550023 | 564 | 80 | 127 | 62 | 7e−45 | |
| 8027 | 2.178485437 | down | 0.0000277 | 0.000281619 | BG790605 | 510 | 138 | 167 | 82 | 5e−82 | cell growth and/or maintenance |
| 8028 | 2.178320966 | down | 0.000134551 | 0.0008503 | AI900670 | 380 | 47 | 121 | 38 | 1e−13 | |
| 8029 | 2.17828393 | down | 0.000800496 | 0.003095595 | AI437547 | 465 | 41 | 49 | 83 | 5e−19 | response to light |
| 8030 | 2.178279344 | down | 0.0000178 | 0.000207749 | CF806262 | 669 | 114 | 236 | 48 | 8e−47 | proteolysis and peptidolysis |
| 8031 | 2.178109346 | down | 0.0000176 | 0.000205986 | AW317399 | 677 | 35 | 61 | 57 | 1e−14 | |
| 8032 | 2.178079688 | down | 0.001779773 | 0.005509826 | BE020227 | 460 | 20 | 40 | 50 | 0.023 | |
| 8033 | 2.178010445 | down | 0.0000368 | 0.000342328 | BE023586 | 385 | 14 | 40 | 35 | 3.1 | transport |
| 8034 | 2.177791626 | down | 0.00000567 | 0.000098 | BI698968 | 410 | 30 | 60 | 50 | 3e−12 | cell adhesion |
| 8035 | 2.177643842 | down | 0.0000531 | 0.000440843 | BQ630696 | 596 | 90 | 108 | 83 | 3e−51 | response to hypoxia |
| 8036 | 2.177296825 | down | 0.0000828 | 0.00060226 | AW598112 | 185 | 14 | 33 | 42 | 5.5 | |
| 8037 | 2.176337963 | down | 0.0000626 | 0.000494847 | AW309535 | 599 | 96 | 109 | 88 | 6e−53 | |
| 8038 | 2.176030939 | down | 0.000238586 | 0.001282942 | AW759802 | 442 | 27 | 46 | 58 | 4e−08 | regulation of transcription |
| 8039 | 2.17459376 | down | 0.0000062 | 0.000103799 | CD390504 | 377 | 14 | 28 | 50 | 0.35 | protein targeting |
| 8040 | 2.174110153 | down | 0.0000362 | 0.000338603 | BE801207 | 447 | 99 | 112 | 88 | 4e−57 | |
| 8041 | 2.173950053 | down | 0.00000917 | 0.000132012 | AW349480 | 468 | 27 | 78 | 34 | 2e−07 | |
| 8042 | 2.17387856 | down | 0.0000239 | 0.000254308 | BE608334 | 493 | 115 | 164 | 70 | 2e−65 | |
| 8043 | 2.17347475 | down | 0.0000531 | 0.000440843 | BU548087 | 656 | 93 | 225 | 41 | 3e−35 | regulation of transcription |
| 8044 | 2.173407278 | down | 0.0000353 | 0.000333289 | AW459596 | 402 | 12 | 37 | 32 | 0.93 | |
| 8045 | 2.17331561 | down | 0.0000891 | 0.000635045 | AW203448 | 363 | 12 | 30 | 40 | 6.0 | proteolysis and peptidolysis |
| 8046 | 2.173170241 | down | 0.0000147 | 0.000182972 | BM731508 | 547 | 92 | 146 | 63 | 5e−49 | |
| 8047 | 2.173126559 | down | 0.0000253 | 0.000264979 | CF806484 | 509 | 93 | 157 | 59 | 2e−45 | electron transport |
| 8048 | 2.17292185 | down | 0.0000121 | 0.000160024 | AW350332 | 637 | 92 | 134 | 68 | 1e−51 | |
| 8049 | 2.172813677 | down | 0.0000484 | 0.000414092 | AW348635 | 763 | 126 | 206 | 61 | 3e−78 | |
| 8050 | 2.172671167 | down | 0.000377511 | 0.001791082 | BQ080654 | 420 | 105 | 113 | 92 | 7e−58 | fumarate metabolism |
| 8051 | 2.172609175 | down | 0.0000323 | 0.000313953 | CD399296 | 684 | 42 | 103 | 40 | 1e−13 | |
| 8052 | 2.172335561 | down | 0.000191072 | 0.001089926 | BI970256 | 703 | 53 | 152 | 34 | 3e−19 | regulation of transcription |
| 8053 | 2.172090335 | down | 0.000339393 | 0.001655248 | BM954903 | 435 | 16 | 28 | 57 | 0.23 | |
| 8054 | 2.171885406 | down | 0.000469655 | 0.002099797 | BU082743 | 465 | 31 | 54 | 57 | 2e−08 | |
| 8055 | 2.171725654 | down | 0.00000518 | 0.0000929 | BG652966 | 458 | 78 | 125 | 62 | 2e−45 | transport |
| 8056 | 2.171648433 | down | 0.000891742 | 0.003358899 | CF808990 | 705 | 165 | 222 | 74 | 2e−92 | aspartate aminotransferase glyoxysoma |
| | | | | | | | | | | | isozyme AAT1 precursor |
| 8057 | 2.171600109 | down | 0.0000224 | 0.000243194 | BM094218 | 580 | 118 | 146 | 80 | 6e−63 | |
| 8058 | 2.171587692 | down | 0.000143599 | 0.00088984 | CD403139 | 587 | 36 | 99 | 36 | 1e−04 | metal ion transport |
| 8059 | 2.17105852 | down | 0.000260466 | 0.001368317 | BE609440 | 639 | 132 | 213 | 61 | 5e−78 | |
| 8060 | 2.17070163 | down | 0.00165503 | 0.005243624 | BU964510 | 620 | 145 | 203 | 71 | 3e−76 | |
| 8061 | 2.170585201 | down | 0.000193982 | 0.001102178 | BE330108 | 671 | 93 | 165 | 56 | 1e−45 | |
| 8062 | 2.170578051 | down | 0.0000966 | 0.000673637 | BU927430 | 457 | 115 | 152 | 75 | 3e−61 | |
| 8063 | 2.170518775 | down | 0.0000164 | 0.000196871 | BE659282 | 485 | 68 | 80 | 85 | 3e−38 | |
| 8064 | 2.169862824 | down | 0.0000395 | 0.00035862 | BU762938 | 601 | 99 | 158 | 62 | 8e−53 | porphyrin biosynthesis |
| 8065 | 2.169611476 | down | 0.000108435 | 0.000728334 | AW568434 | 514 | 53 | 61 | 86 | 5e−26 | |
| 8066 | 2.169492336 | down | 0.00000928 | 0.000132783 | BQ133728 | 541 | 126 | 174 | 72 | 1e−69 | |
| 8067 | 2.169336433 | down | 0.0000115 | 0.000154229 | BQ299018 | 293 | 12 | 27 | 44 | 3.3 | |
| 8068 | 2.169085049 | down | 0.00000642 | 0.000105789 | AW309669 | 680 | 139 | 154 | 90 | 2e−74 | acetyl-CoA biosynthesis |
| 8069 | 2.16894223 | down | 0.000161089 | 0.000966996 | BI972758 | 426 | 62 | 130 | 47 | 9e−27 | response to pest/pathogen/parasite |
| 8070 | 2.168198257 | down | 0.0000061 | 0.00010256 | CD394513 | 552 | 95 | 149 | 63 | 5e−52 | |
| 8071 | 2.167726886 | down | 0.00000822 | 0.000122494 | CD399274 | 684 | 141 | 145 | 97 | 2e−82 | ubiquitin-dependent protein catabolism |
| 8072 | 2.167599747 | down | 0.0000942 | 0.000660695 | BF715908 | 504 | 136 | 167 | 81 | 6e−79 | |
| 8073 | 2.167553347 | down | 0.0000196 | 0.000221357 | BQ611050 | 411 | 26 | 26 | 100 | 2e−09 | microtubule-based process |
| 8074 | 2.167432391 | down | 0.0000633 | 0.000498939 | BI967256 | 584 | 10 | 19 | 52 | 6.0 | metabolism |
| 8075 | 2.167288973 | down | 0.0000184 | 0.000211575 | BU548978 | 660 | 83 | 131 | 63 | 3e−37 | transport |
| 8076 | 2.167092438 | down | 0.00068396 | 0.002757983 | BI971851 | 566 | 103 | 186 | 55 | 5e−55 | biological_process unknown |
| 8077 | 2.166755817 | down | 0.0000288 | 0.000289278 | AW759306 | 497 | 9 | 22 | 40 | 2.0 | response to pathogenic fungi |
| 8078 | 2.166451835 | down | 0.0000122 | 0.000160916 | AW704850 | 510 | 129 | 169 | 76 | 1e−69 | transport |
| 8079 | 2.166433967 | down | 0.000237461 | 0.00127902 | BQ094708 | 572 | 56 | 182 | 30 | 1e−12 | regulation of transcription\, DNA- |
| | | | | | | | | | | | dependent |
| 8080 | 2.166405484 | down | 0.0000627 | 0.000495222 | BE658900 | 704 | 82 | 98 | 83 | 2e−42 | isoprenoid biosynthesis |
| 8081 | 2.166132044 | down | 0.0000164 | 0.000197178 | AI460889 | 457 | 98 | 151 | 64 | 1e−42 | |
| 8082 | 2.166129381 | down | 0.0000196 | 0.000221197 | BU544946 | 556 | 68 | 166 | 40 | 9e−29 | dicarboxylic acid transport |
| 8083 | 2.159879248 | down | 0.0000697 | 0.000533717 | BQ785268 | 559 | 116 | 207 | 56 | 3e−62 | oligopeptide transport |
| 8084 | 2.159220437 | down | 0.0000901 | 0.000639742 | AW233786 | 519 | 97 | 113 | 85 | 6e−53 | |
| 8085 | 2.159085577 | down | 0.0000074 | 0.0001153 | CD393430 | 277 | 15 | 52 | 28 | 1.4 | |
| 8086 | 2.159076498 | down | 0.00000847 | 0.000124852 | CD399859 | 675 | 67 | 89 | 75 | 6e−32 | |
| 8087 | 2.158636572 | down | 0.000692705 | 0.002783104 | BE659955 | 711 | 65 | 140 | 46 | 1e−24 | GAI2, regulation of transcription |
| 8088 | 2.158298001 | down | 0.0000258 | 0.000268562 | BE609367 | 489 | 11 | 18 | 61 | 4.2 | |
| 8089 | 2.158109054 | down | 0.000088 | 0.000629302 | BE821004 | 550 | 26 | 109 | 23 | 0.015 | |
| 8090 | 2.157940578 | down | 0.001399666 | 0.004636462 | BI941740 | 796 | 188 | 259 | 72 | e−102 | galactose metabolism |
| 8091 | 2.157874964 | down | 0.000129939 | 0.000830506 | AW201256 | 447 | 49 | 88 | 55 | 1e−22 | electron transport |
| 8092 | 2.157660789 | down | 0.000669985 | 0.002720394 | AW099852 | 591 | 31 | 98 | 31 | 1e−04 | |
| 8093 | 2.157470828 | down | 0.0000793 | 0.000583974 | BU544404 | 513 | 29 | 50 | 58 | 3e−06 | protein amino acid phosphorylation |
| 8094 | 2.156994877 | down | 0.000047 | 0.000406543 | CD399794 | 581 | 84 | 119 | 70 | 4e−43 | |
| 8095 | 2.156914638 | down | 0.0000509 | 0.00042784 | CF920232 | 481 | 43 | 67 | 64 | 4e−19 | glycolysis |
| 8096 | 2.156756988 | down | 0.0000723 | 0.000546885 | BM523734 | 560 | 21 | 29 | 72 | 9e−06 | trehalose biosynthesis |
| 8097 | 2.156746473 | down | 0.0000117 | 0.000155972 | BQ454151 | 460 | 15 | 39 | 38 | 1.3 | |
| 8098 | 2.156071168 | down | 0.000134493 | 0.000850219 | CD395955 | 493 | 13 | 28 | 46 | 1.1 | nucleotide-sugar transport |
| 8099 | 2.155754002 | down | 0.0000338 | 0.00032389 | CF806460 | 666 | 119 | 165 | 72 | 3e−70 | |
| 8100 | 2.155719857 | down | 0.0000229 | 0.000246847 | CD402535 | 721 | 141 | 167 | 84 | 3e−77 | |
| 8101 | 2.155685716 | down | 0.000520001 | 0.002262764 | BE802793 | 480 | 92 | 160 | 57 | 4e−50 | |
| 8102 | 2.155360785 | down | 0.000203284 | 0.001141304 | CD416202 | 661 | 52 | 93 | 55 | 7e−24 | |
| 8103 | 2.155220146 | down | 0.000387545 | 0.00182579 | BE819900 | 676 | 120 | 166 | 72 | 9e−62 | |
| 8104 | 2.155056439 | down | 0.001683966 | 0.005308919 | BQ272769 | 591 | 148 | 198 | 74 | 6e−83 | |
| 8105 | 2.154731834 | down | 0.000299883 | 0.001518554 | BE020433 | 410 | 80 | 128 | 62 | 5e−32 | extracellular transport |
| 8106 | 2.15438286 | down | 0.0000186 | 0.000213397 | BE658964 | 709 | 106 | 139 | 76 | 4e−57 | |
| 8107 | 2.154257487 | down | 0.000175666 | 0.001028404 | CD402653 | 569 | 82 | 82 | 100 | 1e−40 | nucleosome assembly |
| 8108 | 2.153909049 | down | 0.0000159 | 0.000192946 | BE657218 | 734 | 34 | 49 | 69 | 2e−14 | |
| 8109 | 2.153573475 | down | 0.000783175 | 0.003045988 | BQ453101 | 559 | 73 | 112 | 65 | 3e−38 | carbohydrate metabolism |
| 8110 | 2.153526712 | down | 0.000746848 | 0.002939716 | BM891831 | 547 | 112 | 173 | 64 | 2e−66 | electron transport |
| 8111 | 2.153449245 | down | 0.000194105 | 0.001102712 | CF806642 | 273 | 7 | 17 | 41 | 2.4 | |
| 8112 | 2.153397713 | down | 0.0000485 | 0.000414493 | BU550044 | 607 | 55 | 68 | 80 | 5e−29 | |
| 8113 | 2.153204037 | down | 0.0000324 | 0.000314416 | BU578509 | 581 | 124 | 182 | 68 | 3e−67 | |
| 8114 | 2.15289979 | down | 0.0000402 | 0.000363368 | BQ627692 | 397 | 22 | 54 | 40 | 0.002 | response to sucrose stimulus |
| 8115 | 2.152677613 | down | 0.0000215 | 0.000236092 | CF808630 | 393 | 88 | 103 | 85 | 4e−47 | adenosine salvage pathway |
| 8116 | 2.1525496 | down | 0.00000996 | 0.000139955 | CK606085 | 350 | 57 | 115 | 49 | 2e−22 | defense response |
| 8117 | 2.152275816 | down | 0.0000324 | 0.000314624 | BE821177 | 714 | 113 | 186 | 60 | 8e−61 | cell growth and/or maintenance |
| 8118 | 2.152202618 | down | 0.0000195 | 0.000221197 | BU964440 | 607 | 23 | 83 | 27 | 3.7 | |
| 8119 | 2.151895767 | down | 0.0000116 | 0.000155047 | BG406373 | 428 | 70 | 107 | 65 | 2e−35 | cytidine deamination |
| 8120 | 2.151273527 | down | 0.000129696 | 0.000829659 | BI967315 | 684 | 69 | 102 | 67 | 3e−32 | nucleosome assembly |
| 8121 | 2.150520344 | down | 0.000642982 | 0.002643184 | AW509469 | 675 | 62 | 107 | 57 | 8e−31 | |
| 8122 | 2.150294529 | down | 0.0000154 | 0.000189144 | BQ611255 | 603 | 87 | 170 | 51 | 4e−37 | electron transport |
| 8123 | 2.149383785 | down | 0.0000216 | 0.000236799 | BU551340 | 605 | 64 | 77 | 83 | 4e−33 | tricarboxylic acid cycle |
| 8124 | 2.149325597 | down | 0.0000243 | 0.000257296 | AI856522 | 250 | 11 | 30 | 36 | 1.8 | |
| 8125 | 2.148803191 | down | 0.000159715 | 0.000960894 | BG352531 | 396 | 79 | 100 | 79 | 7e−41 | carbohydrate metabolism |
| 8126 | 2.148772315 | down | 0.0000249 | 0.000262747 | CF805710 | 514 | 72 | 103 | 69 | 5e−37 | |
| 8127 | 2.148265376 | down | 0.000056 | 0.000458643 | BI321448 | 453 | 88 | 147 | 59 | 3e−51 | |
| 8128 | 2.148131653 | down | 0.0000319 | 0.000311046 | CF806563 | 700 | 171 | 216 | 79 | 7e−94 | protein biosynthesis |
| 8129 | 2.147794747 | down | 0.000162148 | 0.000971184 | BF070903 | 371 | 67 | 96 | 69 | 3e−32 | |
| 8130 | 2.147570816 | down | 0.0000117 | 0.000155589 | BU550885 | 694 | 84 | 150 | 56 | 2e−44 | small GTPase mediated signal |
| | | | | | | | | | | | transduction |
| 8131 | 2.146737981 | down | 0.000419754 | 0.001935116 | AI443953 | 524 | 72 | 153 | 47 | 6e−24 | |
| 8132 | 2.145828987 | down | 0.000152506 | 0.000927611 | BE657439 | 789 | 99 | 173 | 57 | 9e−49 | protein amino acid phosphorylation |
| 8133 | 2.145654659 | down | 0.000124744 | 0.000807872 | CF808861 | 481 | 123 | 150 | 82 | 7e−66 | ubiquitin-dependent protein catabolism |
| 8134 | 2.145617629 | down | 0.000559942 | 0.002394457 | BM954238 | 530 | 132 | 179 | 73 | 1e−67 | protein amino acid phosphorylation |
| 8135 | 2.145573756 | down | 0.000730779 | 0.002892815 | BU965258 | 449 | 45 | 54 | 83 | 6e−22 | regulation of transcription\, DNA- |
| | | | | | | | | | | | dependent |
| 8136 | 2.145038315 | down | 0.000178961 | 0.001039446 | BM094295 | 515 | 23 | 49 | 46 | 0.017 | |
| 8137 | 2.144752858 | down | 0.0000535 | 0.000443203 | BG511440 | 498 | 79 | 171 | 46 | 1e−32 | lipid metabolism |
| 8138 | 2.144658871 | down | 0.0000591 | 0.000475752 | BI972743 | 453 | 104 | 142 | 73 | 4e−61 | steroid metabolism |
| 8139 | 2.144282797 | down | 0.0000276 | 0.000280947 | CD411706 | 655 | 29 | 42 | 69 | 4e−14 | response to brassinosteroid stimulus |
| 8140 | 2.143876506 | down | 0.000163017 | 0.000974219 | BI944140 | 470 | 38 | 43 | 88 | 2e−17 | regulation of nitrogen utilization |
| 8141 | 2.143704994 | down | 0.0000181 | 0.000209414 | BG239691 | 464 | 29 | 81 | 35 | 0.005 | |
| 8142 | 2.143460975 | down | 0.000467602 | 0.002093289 | BM139626 | 269 | 79 | 89 | 88 | 1e−41 | |
| 8143 | 2.143063841 | down | 0.0000436 | 0.000386227 | CD398446 | 644 | 23 | 47 | 48 | 2e−08 | lipid transport |
| 8144 | 2.142295052 | down | 0.00000995 | 0.000139905 | AW307590 | | | | | | |
| 8145 | 2.142136329 | down | 0.000221544 | 0.001215318 | BF325451 | 590 | 148 | 196 | 75 | 7e−91 | |
| 8146 | 2.142030971 | down | 0.0000155 | 0.000190225 | CD399965 | 619 | 50 | 138 | 36 | 2e−21 | |
| 8147 | 2.141577018 | down | 0.000104479 | 0.00070912 | CD401290 | 657 | 86 | 122 | 70 | 3e−45 | protein biosynthesis |
| 8148 | 2.141537974 | down | 0.000292639 | 0.001491913 | BI971611 | 562 | 11 | 29 | 37 | 3.2 | |
| 8149 | 2.141532908 | down | 0.000027 | 0.000276788 | CA783780 | 546 | 75 | 172 | 43 | 1e−31 | |
| 8150 | 2.141490032 | down | 0.000102928 | 0.000701492 | BM524557 | 484 | 66 | 102 | 64 | 4e−34 | |
| 8151 | 2.141369253 | down | 0.00094423 | 0.003505701 | AW508411 | 629 | 186 | 209 | 88 | e−111 | sulfate assimilation |
| 8152 | 2.14125533 | down | 0.000191001 | 0.001089769 | BG509382 | 404 | 45 | 50 | 90 | 2e−21 | cell-cell signaling |
| 8153 | 2.140999423 | down | 0.001056335 | 0.003792762 | AI736707 | 560 | 60 | 87 | 68 | 1e−24 | protein amino acid phosphorylation |
| 8154 | 2.140536415 | down | 0.00070467 | 0.00281719 | BI423999 | 542 | 46 | 79 | 58 | 4e−18 | regulation of transcription |
| 8155 | 2.140519629 | down | 0.000066 | 0.000513211 | CF808647 | 566 | 66 | 155 | 42 | 4e−32 | |
| 8156 | 2.093342813 | down | 0.0000148 | 0.000183966 | BQ298665 | 393 | 11 | 19 | 57 | 2.6 | |
| 8157 | 2.093319554 | down | 0.000127241 | 0.000818678 | BQ133844 | 420 | 83 | 138 | 60 | 7e−42 | regulation of transcription\, DNA- |
| | | | | | | | | | | | dependent |
| 8158 | 2.093207397 | down | 0.0000296 | 0.000294882 | BE821823 | 530 | 57 | 130 | 43 | 2e−24 | carbohydrate metabolism |
| 8159 | 2.093062229 | down | 0.00017176 | 0.001012946 | BE803826 | 497 | 29 | 79 | 36 | 8e−05 | |
| 8160 | 2.093053097 | down | 0.000025 | 0.000262771 | BQ629100 | 562 | 19 | 65 | 29 | 5.5 | |
| 8161 | 2.092275261 | down | 0.0000319 | 0.00031075 | BU544073 | 581 | 92 | 158 | 58 | 4e−46 | |
| 8162 | 2.092259763 | down | 0.000126219 | 0.000814899 | BE022115 | 440 | 102 | 146 | 69 | 1e−55 | proteolysis and peptidolysis |
| 8163 | 2.092154207 | down | 0.000097 | 0.000675599 | CF806478 | 510 | 51 | 127 | 40 | 7e−15 | ciliary/flagellar motility |
| 8164 | 2.092129629 | down | 0.00016243 | 0.000972253 | BQ628360 | 167 | 14 | 32 | 43 | 5.5 | |
| 8165 | 2.092114594 | down | 0.000037 | 0.00034358 | BU763922 | 473 | 19 | 65 | 29 | 0.79 | G-protein coupled receptor protein |
| | | | | | | | | | | | signaling pathway |
| 8166 | 2.091459085 | down | 0.000106309 | 0.000717514 | BE475543 | 450 | 117 | 147 | 79 | 1e−65 | |
| 8167 | 2.091294215 | down | 0.0000167 | 0.00019915 | BI320975 | 444 | 24 | 71 | 33 | 3e−06 | protein amino acid phosphorylation |
| 8168 | 2.091113615 | down | 0.0000576 | 0.00046747 | BI471822 | 488 | 100 | 162 | 61 | 1e−49 | |
| 8169 | 2.140047043 | down | 0.0000289 | 0.000289722 | CA937082 | 535 | 79 | 163 | 48 | 1e−26 | Heat shock transcription factor 31, |
| | | | | | | | | | | | regulation of transcription\, DNA- |
| | | | | | | | | | | | dependent |
| 8170 | 2.139888239 | down | 0.000263046 | 0.00137723 | BF424229 | 602 | 151 | 205 | 73 | 1e−86 | protein amino acid phosphorylation |
| 8171 | 2.139859168 | down | 0.0000268 | 0.000275227 | BQ252518 | 569 | 89 | 157 | 56 | 4e−43 | |
| 8172 | 2.139336173 | down | 0.00124947 | 0.004272894 | BG044510 | 681 | 59 | 171 | 34 | 1e−18 | |
| 8173 | 2.13927158 | down | 0.0000149 | 0.000185201 | BE658339 | 391 | 27 | 92 | 29 | 0.004 | |
| 8174 | 2.138863242 | down | 0.000304121 | 0.00153361 | CF807546 | 768 | 184 | 240 | 76 | e−109 | histidine biosynthesis |
| 8175 | 2.138674545 | down | 0.0000347 | 0.000330039 | BU084413 | 588 | 30 | 68 | 44 | 2e−13 | regulation of cell cycle |
| 8176 | 2.138331152 | down | 0.0000439 | 0.000387914 | CD416430 | 261 | 12 | 38 | 31 | 7.2 | protein amino acid phosphorylation |
| 8177 | 2.138319778 | down | 0.0000714 | 0.000542988 | BQ629283 | 392 | 12 | 20 | 60 | 9.8 | apoptosis |
| 8178 | 2.137796924 | down | 0.0000469 | 0.000406221 | CF807730 | 756 | 185 | 239 | 77 | e−108 | epsilon2-COP, protein biosynthesis |
| 8179 | 2.137776081 | down | 0.0000255 | 0.000266283 | CD393440 | 746 | 17 | 50 | 34 | 0.073 | |
| 8180 | 2.137627225 | down | 0.000126057 | 0.000814265 | BG237899 | 377 | 62 | 106 | 58 | 9e−26 | protein amino acid phosphorylation |
| 8181 | 2.137580768 | down | 0.0000353 | 0.000333289 | BF425727 | 475 | 21 | 60 | 35 | 0.46 | |
| 8182 | 2.137396613 | down | 0.0000831 | 0.000603553 | BU083471 | 581 | 118 | 200 | 59 | 5e−55 | |
| 8183 | 2.13738788 | down | 0.000999146 | 0.003648371 | BI943360 | 441 | 75 | 117 | 64 | 1e−32 | glycolysis |
| 8184 | 2.137225892 | down | 0.000130668 | 0.000833888 | BE021850 | 445 | 116 | 148 | 78 | 9e−65 | protein amino acid phosphorylation |
| 8185 | 2.136656256 | down | 0.000604401 | 0.002524311 | BG507675 | 517 | 111 | 158 | 70 | 2e−60 | protein amino acid phosphorylation |
| 8186 | 2.136205539 | down | 0.000743411 | 0.002928641 | CF809008 | 551 | 112 | 182 | 61 | 3e−58 | galactose metabolism |
| 8187 | 2.135611844 | down | 0.0000208 | 0.000230551 | CD408422 | 590 | 72 | 90 | 80 | 4e−35 | cation transport |
| 8188 | 2.135289282 | down | 0.0000383 | 0.000352083 | CF805807 | 330 | 14 | 43 | 32 | 0.23 | phosphate transporter |
| 8189 | 2.135259516 | down | 0.000454948 | 0.002051865 | CD416524 | 695 | 78 | 144 | 54 | 7e−36 | |
| 8190 | 2.134942996 | down | 0.0000542 | 0.000447243 | BQ252463 | 419 | 102 | 138 | 73 | 7e−53 | regulation of transcription |
| 8191 | 2.134713061 | down | 0.000585144 | 0.002471046 | BM885458 | 126 | 10 | 21 | 47 | 5.5 | regulation of transcription\, DNA- |
| | | | | | | | | | | | dependent |
| 8192 | 2.134178568 | down | 0.0000542 | 0.000447122 | BQ473336 | 277 | 12 | 19 | 63 | 3.2 | |
| 8193 | 2.13406982 | down | 0.0000195 | 0.000221197 | BU546185 | 495 | 38 | 112 | 33 | 2e−06 | regulation of transcription |
| 8194 | 2.133638152 | down | 0.00000658 | 0.000107059 | BE824025 | 661 | 52 | 85 | 61 | 2e−24 | |
| 8195 | 2.13318572 | down | 0.0000162 | 0.000195895 | CD396207 | 667 | 75 | 98 | 76 | 7e−38 | |
| 8196 | 2.133153801 | down | 0.0000107 | 0.000146899 | CA851116 | 303 | 15 | 51 | 29 | 1.1 | metabolism |
| 8197 | 2.132947105 | down | 0.0000314 | 0.000307919 | CA938657 | 519 | 63 | 89 | 70 | 1e−31 | |
| 8198 | 2.132768708 | down | 0.0000852 | 0.000614818 | BE211623 | 497 | 83 | 144 | 57 | 1e−44 | electron transport |
| 8199 | 2.132667315 | down | 0.00005 | 0.000422709 | CD397432 | 649 | 33 | 74 | 44 | 7e−05 | |
| 8200 | 2.132199315 | down | 0.000345966 | 0.001678299 | BI320751 | 504 | 127 | 167 | 76 | 9e−70 | |
| 8201 | 2.132018704 | down | 0.000417029 | 0.001926095 | AW348778 | 688 | 83 | 126 | 65 | 4e−45 | |
| 8202 | 2.131754032 | down | 0.0000435 | 0.000385493 | BU080862 | 574 | 155 | 191 | 81 | 2e−84 | |
| 8203 | 2.131480432 | down | 0.000241718 | 0.001295603 | CD399436 | 631 | 64 | 69 | 92 | 9e−32 | protein biosynthesis |
| 8204 | 2.131378686 | down | 0.000036 | 0.000337961 | BG404904 | 326 | 41 | 82 | 50 | 1e−20 | electron transport |
| 8205 | 2.131373577 | down | 0.0000838 | 0.000607126 | BI786698 | 549 | 12 | 27 | 44 | 8.9 | |
| 8206 | 2.131034016 | down | 0.00000672 | 0.000108569 | BU765323 | 422 | 39 | 70 | 55 | 1e−16 | |
| 8207 | 2.130609752 | down | 0.00000718 | 0.000113104 | BE058510 | 349 | 17 | 47 | 36 | 0.020 | protein amino acid phosphorylation |
| 8208 | 2.130215972 | down | 0.000129216 | 0.000827009 | AW306884 | 458 | 68 | 156 | 43 | 4e−27 | |
| 8209 | 2.129927429 | down | 0.0000547 | 0.000450203 | BE806947 | 349 | 14 | 32 | 43 | 5.5 | regulation of transcription |
| 8210 | 2.129840473 | down | 0.001170957 | 0.004077051 | AW733177 | 441 | 11 | 35 | 31 | 2.6 | |
| 8211 | 2.129676888 | down | 0.0000141 | 0.000177559 | CA852778 | 593 | 50 | 118 | 42 | 6e−18 | response to pathogenic bacteria |
| | | | | | | | | | | | (incompatible interaction) |
| 8212 | 2.129515152 | down | 0.00014988 | 0.000915785 | L01430.1 | | | | | | calmodulin |
| 8213 | 2.129466773 | down | 0.0000343 | 0.000327554 | BE607553 | 476 | 56 | 133 | 42 | 4e−21 | |
| 8214 | 2.129382256 | down | 0.000307844 | 0.001546371 | CD417960 | 583 | 12 | 22 | 54 | 0.53 | |
| 8215 | 2.129349529 | down | 0.0000206 | 0.000228634 | BU548345 | 662 | 121 | 144 | 84 | 2e−67 | lipid metabolism |
| 8216 | 2.129337688 | down | 0.0000111 | 0.000150465 | BF424010 | 452 | 19 | 50 | 38 | 2e−07 | basic amino acid transport |
| 8217 | 2.129223197 | down | 0.0000526 | 0.000438086 | BQ611819 | 607 | 39 | 113 | 34 | 1e−09 | regulation of transcription\, DNA- |
| | | | | | | | | | | | dependent |
| 8218 | 2.129154502 | down | 0.0000431 | 0.000382955 | AW203420 | 367 | 43 | 113 | 38 | 2e−13 | |
| 8219 | 2.128579378 | down | 0.0000345 | 0.000329083 | CD393829 | 262 | 27 | 44 | 61 | 2e−08 | regulation of transcription |
| 8220 | 2.128136169 | down | 0.0000203 | 0.000226605 | BM271030 | 608 | 37 | 55 | 67 | 3e−17 | |
| 8221 | 2.128131832 | down | 0.000163246 | 0.000974811 | BQ299038 | 579 | 39 | 59 | 66 | 1e−20 | transport |
| 8222 | 2.127043943 | down | 0.0000136 | 0.000174054 | AW351148 | 784 | 96 | 166 | 57 | 2e−48 | |
| 8223 | 2.126713828 | down | 0.0000648 | 0.000507659 | CF809241 | 573 | 55 | 115 | 47 | 6e−21 | |
| 8224 | 2.126542476 | down | 0.00000768 | 0.000117626 | BE658878 | 299 | 16 | 49 | 32 | 4.2 | cell wall biosynthesis |
| 8225 | 2.126516686 | down | 0.0000629 | 0.000496787 | AW830258 | 408 | 26 | 91 | 28 | 0.43 | |
| 8226 | 2.125940458 | down | 0.0000782 | 0.000577855 | BQ296858 | 530 | 56 | 71 | 78 | 8e−30 | |
| 8227 | 2.125691927 | down | 0.00000759 | 0.000117067 | CD400233 | 589 | 71 | 110 | 64 | 1e−39 | threonine catabolism |
| 8228 | 2.125456977 | down | 0.0000479 | 0.000411408 | BE346228 | 513 | 25 | 94 | 26 | 7.9 | |
| 8229 | 2.125313184 | down | 0.000053 | 0.000440695 | BE022504 | 372 | 10 | 24 | 41 | 1.00 | protein amino acid phosphorylation |
| 8230 | 2.125118254 | down | 0.0000201 | 0.000224309 | BF425401 | 511 | 28 | 78 | 35 | 9e−09 | |
| 8231 | 2.125055483 | down | 0.0000134 | 0.000171039 | AW423408 | 384 | 19 | 24 | 79 | 2e−08 | |
| 8232 | 2.125027451 | down | 0.0000173 | 0.000204023 | BE020749 | 545 | 40 | 55 | 72 | 2e−20 | |
| 8233 | 2.125005709 | down | 0.0000235 | 0.000252134 | BE821780 | 723 | 102 | 200 | 51 | 3e−57 | carbohydrate metabolism |
| 8234 | 2.124885274 | down | 0.0000485 | 0.000414391 | BG725741 | 372 | 29 | 39 | 74 | 1e−08 | regulation of transcription |
| 8235 | 2.1245109 | down | 0.0000146 | 0.000181757 | BU926281 | 551 | 10 | 43 | 23 | 1.4 | |
| 8236 | 2.124453766 | down | 0.000403865 | 0.001882169 | CD397785 | 532 | 50 | 67 | 74 | 1e−23 | sterol biosynthesis |
| 8237 | 2.124423516 | down | 0.000109804 | 0.000734773 | AI736414 | 506 | 65 | 154 | 42 | 3e−24 | DNA replication initiation |
| 8238 | 2.124178384 | down | 0.001762305 | 0.005477023 | BM519618 | 563 | 134 | 187 | 71 | 1e−78 | cell-matrix adhesion |
| 8239 | 2.123842995 | down | 0.0000515 | 0.000431871 | BM107783 | 554 | 25 | 40 | 62 | 2e−07 | regulation of gene expression\, |
| | | | | | | | | | | | epigenetic |
| 8240 | 2.123482302 | down | 0.0000109 | 0.000149195 | BE820709 | 253 | 11 | 13 | 84 | 0.50 | |
| 8241 | 2.123355216 | down | 0.0000137 | 0.000174178 | BG046191 | 337 | 35 | 51 | 68 | 8e−15 | |
| 8242 | 2.122884047 | down | 0.0000492 | 0.000418717 | AI442440 | 590 | 15 | 36 | 41 | 0.013 | Sali3-2, response to dessication |
| 8243 | 2.122812099 | down | 0.0000309 | 0.0003044 | AW277890 | 530 | 130 | 176 | 73 | 4e−71 | |
| 8244 | 2.122721232 | down | 0.0000767 | 0.000570052 | BU762206 | 634 | 144 | 211 | 68 | 2e−81 | protein amino acid phosphorylation |
| 8245 | 2.122408548 | down | 0.0000337 | 0.000323412 | AW310069 | 675 | 102 | 136 | 75 | 6e−58 | |
| 8246 | 2.122229094 | down | 0.000047 | 0.000406543 | BG725466 | 429 | 45 | 98 | 45 | 5e−14 | electron transport |
| 8247 | 2.121692872 | down | 0.0000965 | 0.000673255 | CF808979 | 730 | 78 | 240 | 32 | 2e−36 | |
| 8248 | 2.121571169 | down | 0.000588913 | 0.002480278 | BU550425 | 665 | 89 | 153 | 58 | 5e−49 | carbohydrate metabolism |
| 8249 | 2.121425842 | down | 0.000813064 | 0.003133462 | BU089703 | 671 | 71 | 170 | 41 | 5e−29 | |
| 8250 | 2.121387491 | down | 0.000184481 | 0.001062642 | CF806563 | 700 | 171 | 216 | 79 | 7e−94 | protein biosynthesis |
| 8251 | 2.121379063 | down | 0.0000385 | 0.000352764 | CA802940 | 215 | 13 | 33 | 39 | 2.5 | regulation of transcription\, DNA- |
| | | | | | | | | | | | dependent |
| 8252 | 2.121207834 | down | 0.0000275 | 0.000280629 | AW761383 | 385 | 19 | 24 | 79 | 0.001 | transport |
| 8253 | 2.120926181 | down | 0.001502115 | 0.004877464 | BM086167 | 571 | 145 | 170 | 85 | 1e−80 | signal transduction |
| 8254 | 2.120682689 | down | 0.001383444 | 0.004598521 | CF806634 | 688 | 112 | 187 | 59 | 5e−53 | D-xylose metabolism |
| 8255 | 2.120669921 | down | 0.001517142 | 0.004912686 | BM886578 | 423 | 13 | 13 | 100 | 0.61 | |
| 8256 | 2.116019365 | down | 0.000924588 | 0.003451173 | CD402329 | 580 | 13 | 34 | 38 | 5.8 | Lipoxygenase (lox7) |
| 8257 | 2.115936949 | down | 0.0000189 | 0.000216039 | AI736575 | 395 | 18 | 67 | 26 | 0.52 | electron transport |
| 8258 | 2.115895945 | down | 0.0000309 | 0.000304499 | CK605912 | 623 | 68 | 102 | 66 | 1e−36 | electron transport |
| 8259 | 2.115688117 | down | 0.00000624 | 0.000104291 | AF243376.1 | | | | | | glutathione S-transferase GST 21 |
| 8260 | 2.115468631 | down | 0.0000826 | 0.000601335 | AW349891 | 598 | 56 | 93 | 60 | 1e−25 | |
| 8261 | 2.115410112 | down | 0.00061719 | 0.002562927 | CA785287 | 629 | 89 | 215 | 41 | 3e−36 | |
| 8262 | 2.115351503 | down | 0.0000553 | 0.000454382 | CD408714 | 599 | 52 | 58 | 89 | 1e−23 | glycolysis |
| 8263 | 2.115171613 | down | 0.0000735 | 0.000554085 | BU544031 | 512 | 74 | 156 | 47 | 1e−33 | protein amino acid phosphorylation |
| 8264 | 2.11495656 | down | 0.000029 | 0.000290681 | BF597697 | 426 | 134 | 142 | 94 | 2e−73 | |
| 8265 | 2.114934011 | down | 0.00126548 | 0.004314308 | BI942102 | 666 | 84 | 95 | 88 | 8e−44 | response to stress |
| 8266 | 2.114858492 | down | 0.0000224 | 0.000242928 | BG157539 | 525 | 17 | 23 | 73 | 7e−05 | negative regulation of CDK activity |
| 8267 | 2.114750028 | down | 0.000197472 | 0.00111678 | BI941775 | 503 | 12 | 16 | 75 | 0.11 | |
| 8268 | 2.114542524 | down | 0.0000476 | 0.000409872 | BU577047 | 554 | 53 | 136 | 38 | 3e−19 | |
| 8269 | 2.114497154 | down | 0.0000461 | 0.000401182 | AW351021 | 354 | 30 | 36 | 83 | 4e−11 | |
| 8270 | 2.114483137 | down | 0.0000165 | 0.000197839 | CD415272 | 533 | 60 | 79 | 75 | 8e−32 | |
| 8271 | 2.114334264 | down | 0.0000909 | 0.000644904 | CD405024 | 516 | 83 | 106 | 78 | 3e−44 | glycine catabolism |
| 8272 | 2.114194487 | down | 0.0000509 | 0.000428124 | BM085386 | 513 | 97 | 153 | 63 | 1e−49 | |
| 8273 | 2.114055264 | down | 0.0000354 | 0.000333289 | BE823020 | 599 | 51 | 69 | 73 | 1e−25 | G-protein coupled receptor protein |
| | | | | | | | | | | | signaling pathway |
| 8274 | 2.113955044 | down | 0.00000729 | 0.000114115 | CD404614 | 436 | 10 | 32 | 31 | 1.5 | metabolism |
| 8275 | 2.113741351 | down | 0.0000235 | 0.000252134 | BI942964 | 502 | 133 | 149 | 89 | 5e−76 | methionine metabolism |
| 8276 | 2.113549411 | down | 0.0000741 | 0.000557159 | BE822605 | 659 | 89 | 106 | 83 | 1e−49 | |
| 8277 | 2.11342606 | down | 0.0000524 | 0.000436773 | CD403248 | 469 | 15 | 48 | 31 | 0.78 | proteolysis and peptidolysis |
| 8278 | 2.113340427 | down | 0.00000905 | 0.000130761 | BF009264 | 452 | 63 | 119 | 52 | 2e−31 | protein amino acid phosphorylation |
| 8279 | 2.112923844 | down | 0.000383264 | 0.001811216 | BQ452998 | 574 | 116 | 164 | 70 | 7e−64 | protein modification |
| 8280 | 2.112873196 | down | 0.000148003 | 0.000907859 | BF424755 | 397 | 35 | 78 | 44 | 8e−13 | |
| 8281 | 2.112838587 | down | 0.0000434 | 0.000384728 | BQ081240 | 520 | 44 | 80 | 55 | 3e−19 | |
| 8282 | 2.112639175 | down | 0.0000593 | 0.000476504 | BI787706 | 595 | 146 | 198 | 73 | 4e−81 | protein amino acid phosphorylation |
| 8283 | 2.112393691 | down | 0.000189946 | 0.001085149 | CA800675 | 737 | 80 | 237 | 33 | 9e−24 | apoptosis |
| 8284 | 2.112252745 | down | 0.0000268 | 0.000275505 | BM954335 | 459 | 15 | 42 | 35 | 1.3 | |
| 8285 | 2.112244616 | down | 0.0000831 | 0.000603627 | BG154957 | 528 | 107 | 141 | 75 | 1e−58 | regulation of transcription |
| 8286 | 2.112208187 | down | 0.000213205 | 0.001182291 | BU546227 | 542 | 32 | 48 | 66 | 5e−14 | electron transport |
| 8287 | 2.112183598 | down | 0.0000188 | 0.000214992 | BQ630502 | 637 | 145 | 211 | 68 | 4e−86 | |
| 8288 | 2.112141622 | down | 0.000115482 | 0.000762195 | CA784607 | 433 | 49 | 71 | 69 | 3e−26 | carbohydrate biosynthesis |
| 8289 | 2.112087324 | down | 0.000248127 | 0.001320744 | AI460487 | 181 | 10 | 32 | 31 | 9.4 | |
| 8290 | 2.111987272 | down | 0.000272242 | 0.001411799 | BM309522 | 483 | 97 | 154 | 62 | 1e−50 | |
| 8291 | 2.111966724 | down | 0.000701669 | 0.002810001 | BG155453 | | | | | | |
| 8292 | 2.111805809 | down | 0.0000315 | 0.000308373 | AW432668 | 530 | 112 | 176 | 63 | 7e−66 | electron transport |
| 8293 | 2.111654532 | down | 0.00013997 | 0.00087325 | BQ081489 | 423 | 86 | 102 | 84 | 1e−42 | intracellular protein transport |
| 8294 | 2.111229236 | down | 0.000104396 | 0.00070912 | BQ627927 | 494 | 27 | 56 | 48 | 5e−09 | |
| 8295 | 2.111011052 | down | 0.000169074 | 0.001000401 | AI416901 | 447 | 26 | 88 | 29 | 2.6 | electron transport |
| 8296 | 2.110875225 | down | 0.000016 | 0.000193957 | AW278714 | 281 | 16 | 32 | 50 | 6e−04 | aromatic compound metabolism |
| 8297 | 2.110718464 | down | 0.0000376 | 0.000347409 | BU548081 | 619 | 84 | 96 | 87 | 2e−47 | negative regulation of flower |
| | | | | | | | | | | | development |
| 8298 | 2.110522768 | down | 0.000381373 | 0.001805441 | BM094052 | 541 | 114 | 171 | 66 | 1e−61 | |
| 8299 | 2.109996065 | down | 0.0000793 | 0.000583841 | BQ473578 | 587 | 63 | 122 | 51 | 4e−28 | metal ion transport |
| 8300 | 2.109931142 | down | 0.00000643 | 0.000105803 | CF805817 | 175 | 26 | 34 | 76 | 8e−10 | lipid catabolism |
| 8301 | 2.1097998 | down | 0.0000398 | 0.000360228 | BM107696 | 573 | 58 | 134 | 43 | 2e−26 | electron transport |
| 8302 | 2.109459793 | down | 0.001155891 | 0.0040373 | BG046670 | 428 | 37 | 42 | 88 | 2e−16 | |
| 8303 | 2.109420929 | down | 0.000966309 | 0.003563056 | CA799183 | 761 | 156 | 225 | 69 | 1e−89 | |
| 8304 | 2.109235216 | down | 0.0000958 | 0.000669463 | BG238851 | 667 | 89 | 204 | 43 | 9e−30 | |
| 8305 | 2.108493578 | down | 0.000017 | 0.000201413 | AI441130 | 357 | 76 | 115 | 66 | 2e−39 | protein amino acid phosphorylation |
| 8306 | 2.108038193 | down | 0.000136941 | 0.000861209 | BE822982 | 511 | 18 | 44 | 40 | 0.008 | actin cytoskeleton organization and |
| | | | | | | | | | | | biogenesis |
| 8307 | 2.107733809 | down | 0.0000078 | 0.000118925 | BG047538 | 372 | 15 | 22 | 68 | 0.011 | D-ribose metabolism |
| 8308 | 2.107514286 | down | 0.0000254 | 0.000265557 | CD414002 | 478 | 11 | 37 | 29 | 0.28 | Cyclin-dependent kinases regulatory |
| | | | | | | | | | | | subunit, sodium ion transport |
| 8309 | 2.107430919 | down | 0.0000291 | 0.000291165 | CD418280 | 710 | 90 | 139 | 64 | 1e−48 | |
| 8310 | 2.107232161 | down | 0.000337714 | 0.001649744 | BI497772 | 539 | 79 | 92 | 85 | 2e−39 | negative regulation of flower |
| | | | | | | | | | | | development |
| 8311 | 2.10721452 | down | 0.000230873 | 0.001252752 | AI900398 | 456 | 59 | 133 | 44 | 6e−27 | cellulose biosynthesis |
| 8312 | 2.107035437 | down | 0.000210411 | 0.00117025 | BF596273 | 421 | 54 | 130 | 41 | 3e−23 | |
| 8313 | 2.107018874 | down | 0.000126361 | 0.000815393 | BG352722 | 616 | 34 | 64 | 53 | 4e−15 | |
| 8314 | 2.106719864 | down | 0.000226414 | 0.001233714 | AW099997 | 424 | 36 | 112 | 32 | 2e−08 | |
| 8315 | 2.106497211 | down | 0.0000618 | 0.00049022 | BG352722 | 616 | 34 | 64 | 53 | 4e−15 | |
| 8316 | 2.106431568 | down | 0.0000566 | 0.000461949 | CD396420 | 288 | 6 | 12 | 50 | 2.4 | |
| 8317 | 2.106427345 | down | 0.0000719 | 0.000544985 | CD416292 | 693 | 123 | 214 | 57 | 3e−54 | |
| 8318 | 2.106195354 | down | 0.000143421 | 0.000889469 | BM523077 | 573 | 78 | 157 | 49 | 9e−38 | regulation of transcription |
| 8319 | 2.106188943 | down | 0.0000325 | 0.000314635 | BG044010 | 623 | 53 | 133 | 39 | 4e−22 | |
| 8320 | 2.105503296 | down | 0.0000852 | 0.000614818 | CD395892 | 314 | 8 | 17 | 47 | 9.3 | |
| 8321 | 2.104735908 | down | 0.0000123 | 0.000161302 | BI471612 | 421 | 50 | 87 | 57 | 4e−21 | |
| 8322 | 2.104286822 | down | 0.0000186 | 0.000213297 | BE023264 | 593 | 108 | 221 | 48 | 7e−38 | |
| 8323 | 2.103894429 | down | 0.0000235 | 0.000252208 | BQ081060 | 324 | 9 | 17 | 52 | 1.1 | |
| 8324 | 2.103717299 | down | 0.0000102 | 0.000142275 | BU546768 | 524 | 54 | 119 | 45 | 2e−17 | |
| 8325 | 2.103625394 | down | 0.000874581 | 0.003311172 | AW184945 | 504 | 121 | 167 | 72 | 8e−69 | carbohydrate metabolism |
| 8326 | 2.103537644 | down | 0.000322173 | 0.001596113 | CA801440 | 602 | 92 | 193 | 47 | 8e−38 | |
| 8327 | 2.103310627 | down | 0.000058 | 0.000469661 | BE022612 | 484 | 30 | 36 | 83 | 3e−10 | |
| 8328 | 2.103039376 | down | 0.0000392 | 0.00035708 | CA820153 | | | | | | |
| 8329 | 2.102934147 | down | 0.0000238 | 0.000253843 | CD418256 | 539 | 31 | 51 | 60 | 6e−12 | DNA replication |
| 8330 | 2.102846732 | down | 0.000309887 | 0.001554562 | BI944709 | 477 | 74 | 95 | 77 | 4e−44 | metabolism |
| 8331 | 2.102633416 | down | 0.0000184 | 0.00021144 | BU550110 | 488 | 12 | 50 | 24 | 2.5 | |
| 8332 | 2.102620729 | down | 0.000979315 | 0.00360148 | CD401409 | 279 | 16 | 60 | 26 | 9.3 | |
| 8333 | 2.102325221 | down | 0.000256137 | 0.001352188 | CF807077 | 471 | 58 | 90 | 64 | 4e−29 | |
| 8334 | 2.10220543 | down | 0.0000368 | 0.000342685 | AW203260 | 463 | 83 | 100 | 83 | 3e−46 | |
| 8335 | 2.101982173 | down | 0.001075418 | 0.003844431 | BI942654 | 605 | 179 | 200 | 89 | e−103 | ciliary/flagellar motility |
| 8336 | 2.101839417 | down | 0.0000516 | 0.000432129 | CF805641 | 546 | 93 | 153 | 60 | 1e−42 | metabolism |
| 8337 | 2.101740533 | down | 0.0000138 | 0.000174776 | BG508349 | 525 | 39 | 69 | 56 | 2e−16 | |
| 8338 | 2.101654166 | down | 0.000627729 | 0.002596247 | BF068503 | 248 | 61 | 82 | 74 | 3e−31 | flower development |
| 8339 | 2.101635771 | down | 0.001263558 | 0.004308926 | AW132910 | 433 | 18 | 51 | 35 | 0.50 | |
| 8340 | 2.101587153 | down | 0.0000196 | 0.000221197 | U66836.1 | | | | | | RecA/Rad51/DMC1-like protein |
| 8341 | 2.101309044 | down | 0.000125548 | 0.000812239 | CD392230 | 635 | 66 | 90 | 73 | 4e−33 | |
| 8342 | 2.101174594 | down | 0.00000843 | 0.000124488 | CD416083 | 661 | 84 | 128 | 65 | 4e−44 | lipid metabolism |
| 8343 | 2.101160179 | down | 0.000059 | 0.000475076 | BF425858 | 441 | 38 | 64 | 59 | 6e−16 | electron transport |
| 8344 | 2.101129893 | down | 0.0000347 | 0.000329866 | CF806380 | 720 | 195 | 232 | 84 | e−109 | SGF14C |
| 8345 | 2.101112177 | down | 0.000182697 | 0.001055319 | BI787655 | 545 | 137 | 182 | 75 | 1e−81 | |
| 8346 | 2.100962269 | down | 0.0000267 | 0.000274647 | CF807151 | 532 | 78 | 172 | 45 | 9e−34 | |
| 8347 | 2.100861491 | down | 0.000150087 | 0.000916605 | CD392230 | 635 | 66 | 90 | 73 | 4e−33 | |
| 8348 | 2.10083309 | down | 0.0000653 | 0.000509616 | CA937407 | 509 | 13 | 27 | 48 | 2.0 | |
| 8349 | 2.100684863 | down | 0.000342938 | 0.001668298 | BM526594 | 571 | 53 | 65 | 81 | 2e−25 | phototropism |
| 8350 | 2.100592521 | down | 0.0000252 | 0.000264316 | BE348045 | 730 | 112 | 154 | 72 | 8e−59 | regulation of cell cycle |
| 8351 | 2.100266578 | down | 0.000445869 | 0.002020349 | BG652339 | 482 | 116 | 160 | 72 | 9e−67 | glycolysis |
| 8352 | 2.100199567 | down | 0.00015017 | 0.000916817 | BI973641 | 560 | 107 | 142 | 75 | 1e−60 | anion transport |
| 8353 | 2.100127767 | down | 0.0000163 | 0.000196399 | BF068458 | 355 | 18 | 46 | 39 | 3.3 | electron transport |
| 8354 | 2.100089739 | down | 0.0000366 | 0.000341899 | BU549028 | 553 | 63 | 80 | 78 | 2e−31 | transport |
| 8355 | 2.09987004 | down | 0.0000125 | 0.00016339 | BG510932 | 456 | 26 | 54 | 48 | 1e−04 | regulation of transcription\, DNA- |
| | | | | | | | | | | | dependent |
| 8356 | 2.099560041 | down | 0.0000427 | 0.000380213 | BU578044 | 423 | 16 | 39 | 41 | 2e−04 | response to pathogenic bacteria |
| | | | | | | | | | | | (incompatible interaction) |
| 8357 | 2.099498882 | down | 0.0000552 | 0.000453123 | CF805652 | 562 | 142 | 151 | 94 | 4e−83 | 40S ribosomal S4 protein |
| 8358 | 2.099422695 | down | 0.0000672 | 0.000520212 | BU546067 | 552 | 62 | 128 | 48 | 5e−32 | very-long-chain fatty acid metabolism |
| 8359 | 2.099280371 | down | 0.000135372 | 0.000853624 | BQ253870 | 487 | 84 | 144 | 58 | 2e−43 | regulation of transcription |
| 8360 | 2.099269979 | down | 0.00010483 | 0.000710592 | AW278528 | 579 | 33 | 47 | 70 | 8e−15 | |
| 8361 | 2.0990716 | down | 0.0000351 | 0.00033256 | AI938052 | 203 | 12 | 28 | 42 | 0.17 | proteolysis and peptidolysis |
| 8362 | 2.098941195 | down | 0.001173271 | 0.004082463 | AI461110 | 438 | 83 | 114 | 72 | 7e−43 | protein amino acid phosphorylation |
| 8363 | 2.098938291 | down | 0.000053 | 0.000440381 | CD410657 | 498 | 77 | 100 | 77 | 3e−42 | |
| 8364 | 2.098868004 | down | 0.0000779 | 0.000576079 | BQ629510 | 537 | 63 | 140 | 45 | 3e−23 | regulation of transcription\, DNA- |
| | | | | | | | | | | | dependent |
| 8365 | 2.098369034 | down | 0.0000137 | 0.000174749 | AB083032.1 | | | | | | syringolide-induced protein B13-1-1 |
| 8366 | 2.098016417 | down | 0.0000167 | 0.000198784 | BU546655 | 639 | 159 | 167 | 95 | 4e−92 | microtubule-based process |
| 8367 | 2.098008792 | down | 0.0000878 | 0.000628608 | BM732148 | 564 | 33 | 91 | 36 | 8e−07 | regulation of translation |
| 8368 | 2.09784072 | down | 0.0000324 | 0.00031421 | BI971007 | 802 | 94 | 189 | 49 | 2e−38 | |
| 8369 | 2.097747628 | down | 0.000218165 | 0.001201999 | BQ742026 | 592 | 137 | 199 | 68 | 2e−72 | electron transport |
| 8370 | 2.097613824 | down | 0.000296465 | 0.0015057 | BI972851 | 258 | 32 | 38 | 84 | 4e−11 | ossification |
| 8371 | 2.097487907 | down | 0.0000999 | 0.000687789 | AW704626 | 518 | 115 | 174 | 66 | 7e−63 | intracellular protein transport |
| 8372 | 2.097268226 | down | 0.0000132 | 0.000169651 | BG157586 | 452 | 36 | 56 | 64 | 2e−17 | glycolipid biosynthesis |
| 8373 | 2.096758134 | down | 0.000255049 | 0.001347482 | AI495608 | 524 | 133 | 172 | 77 | 4e−71 | proteolysis and peptidolysis |
| 8374 | 2.096695473 | down | 0.00000917 | 0.000132012 | CD396509 | 260 | 11 | 37 | 29 | 1.5 | |
| 8375 | 2.09655397 | down | 0.0000506 | 0.0004262 | BU091330 | 532 | 74 | 181 | 40 | 9e−29 | protein amino acid phosphorylation |
| 8376 | 2.096481099 | down | 0.000087 | 0.000624443 | BF068700 | 425 | 114 | 141 | 80 | 6e−66 | histidine biosynthesis |
| 8377 | 2.095485607 | down | 0.001743195 | 0.005435059 | BM308144 | 593 | 22 | 50 | 44 | 0.017 | |
| 8378 | 2.095107433 | down | 0.001254374 | 0.004286159 | BG405362 | 342 | 78 | 103 | 75 | 1e−43 | |
| 8379 | 2.095093921 | down | 0.0000269 | 0.000276494 | CD404253 | 627 | 37 | 103 | 35 | 1e−11 | |
| 8380 | 2.094523616 | down | 0.000405481 | 0.001888297 | BF008984 | 417 | 101 | 138 | 73 | 9e−58 | transport |
| 8381 | 2.094442407 | down | 0.00000847 | 0.000124852 | BE347552 | 398 | 17 | 66 | 25 | 0.41 | |
| 8382 | 2.094021759 | down | 0.0000165 | 0.000197839 | CD394725 | 638 | 41 | 106 | 38 | 3e−14 | proteolysis and peptidolysis |
| 8383 | 2.093915935 | down | 0.0000578 | 0.000468828 | AI437791 | 412 | 55 | 136 | 40 | 1e−23 | metabolism |
| 8384 | 2.093857846 | down | 0.0000486 | 0.000414541 | AW568239 | 422 | 102 | 139 | 73 | 6e−57 | electron transport |
| 8385 | 2.093760407 | down | 0.0000165 | 0.000197527 | BI969344 | 770 | 31 | 52 | 59 | 4e−10 | |
| 8386 | 2.093552779 | down | 0.000160218 | 0.000963153 | CD401290 | 657 | 86 | 122 | 70 | 3e−45 | protein biosynthesis |
| 8387 | 2.09355224 | down | 0.0000699 | 0.000535287 | BQ740845 | 431 | 13 | 36 | 36 | 2.5 | |
| 8388 | 2.090616565 | down | 0.0000208 | 0.000230646 | BQ611039 | 566 | 38 | 79 | 48 | 3e−17 | |
| 8389 | 2.08957271 | down | 0.0000234 | 0.000250673 | BQ079942 | 179 | 11 | 34 | 32 | 7.2 | |
| 8390 | 2.089309526 | down | 0.0000253 | 0.000264979 | BM525153 | 492 | 9 | 18 | 50 | 0.66 | electron transport |
| 8391 | 2.089029112 | down | 0.000443806 | 0.002014233 | AW704196 | 603 | 157 | 180 | 87 | 8e−88 | |
| 8392 | 2.088716506 | down | 0.0000222 | 0.000241401 | AW349309 | 291 | 30 | 45 | 66 | 3e−15 | |
| 8393 | 2.088680612 | down | 0.000327045 | 0.001614542 | BU964909 | 629 | 125 | 206 | 60 | 3e−77 | proteolysis and peptidolysis |
| 8394 | 2.088412239 | down | 0.000213614 | 0.001183686 | BG881900 | 534 | 142 | 178 | 79 | 6e−86 | proteolysis and peptidolysis |
| 8395 | 2.088283935 | down | 0.0000482 | 0.000413264 | BM886833 | 525 | 13 | 34 | 38 | 4.8 | transport |
| 8396 | 2.088128312 | down | 0.0000685 | 0.000527155 | AW349683 | 507 | 33 | 87 | 37 | 6e−10 | |
| 8397 | 2.087783751 | down | 0.000104709 | 0.000710154 | AW703709 | 572 | 24 | 55 | 43 | 2e−05 | |
| 8398 | 2.087782082 | down | 0.000348358 | 0.001687462 | BG154593 | 535 | 100 | 158 | 63 | 1e−55 | |
| 8399 | 2.087673066 | down | 0.0000746 | 0.000559293 | BE659002 | 511 | 99 | 100 | 99 | 1e−51 | |
| 8400 | 2.087621564 | down | 0.0000616 | 0.00048928 | CD397282 | 592 | 95 | 105 | 90 | 3e−54 | protein biosynthesis |
| 8401 | 2.087242414 | down | 0.0000179 | 0.000207812 | CF806460 | 666 | 119 | 165 | 12 | 3e−70 | |
| 8402 | 2.086924619 | down | 0.000191913 | 0.001093565 | BG156735 | 206 | 14 | 41 | 34 | 3.3 | |
| 8403 | 2.086874107 | down | 0.0000686 | 0.000528121 | CD394552 | 709 | 51 | 148 | 34 | 1e−16 | |
| 8404 | 2.086840279 | down | 0.0000669 | 0.00051895 | BU764593 | 551 | 16 | 29 | 55 | 6e−06 | |
| 8405 | 2.086811669 | down | 0.000232845 | 0.001261114 | BU547726 | 604 | 14 | 44 | 31 | 0.74 | phospholipid biosynthesis |
| 8406 | 2.086803966 | down | 0.0000494 | 0.000419782 | BQ295808 | 578 | 41 | 76 | 53 | 5e−15 | |
| 8407 | 2.086751219 | down | 0.0000762 | 0.000567447 | CA785837 | 599 | 99 | 115 | 86 | 7e−57 | |
| 8408 | 2.086695469 | down | 0.0000386 | 0.000352911 | AI966502 | 297 | 34 | 49 | 69 | 3e−13 | metabolism |
| 8409 | 2.086573797 | down | 0.0000971 | 0.000675669 | BE210499 | 535 | 87 | 130 | 66 | 7e−47 | |
| 8410 | 2.086627096 | down | 0.0000172 | 0.000203165 | AW311155 | 314 | 16 | 61 | 26 | 2.4 | cell proliferation |
| 8411 | 2.08618929 | down | 0.000558409 | 0.002389532 | CF806746 | 725 | 150 | 183 | 81 | 8e−88 | |
| 8412 | 2.086034065 | down | 0.000153129 | 0.000930047 | AW734930 | 391 | 83 | 114 | 72 | 4e−48 | DNA repair |
| 8413 | 2.085966415 | down | 0.000100091 | 0.000688624 | BE058124 | 615 | 164 | 203 | 80 | 1e−96 | |
| 8414 | 2.085739805 | down | 0.0000412 | 0.000370016 | BI787992 | 361 | 20 | 26 | 76 | 4e−06 | |
| 8415 | 2.085739115 | down | 0.0000215 | 0.000235852 | AW396170 | 311 | 21 | 27 | 77 | 2e−06 | |
| 8416 | 2.085555191 | down | 0.000258307 | 0.001360778 | BG405805 | 598 | 49 | 69 | 71 | 5e−21 | |
| 8417 | 2.085534092 | down | 0.0000294 | 0.000293499 | BI971041 | 728 | 40 | 102 | 39 | 3e−11 | protein amino acid phosphorylation |
| 8418 | 2.08543567 | down | 0.0000496 | 0.000420793 | BQ610855 | 455 | 44 | 47 | 93 | 3e−22 | brassinosteroid biosynthesis |
| 8419 | 2.08542416 | down | 0.0000236 | 0.000252555 | BU082147 | 561 | 38 | 133 | 28 | 3e−05 | |
| 8420 | 2.085108015 | down | 0.0000353 | 0.000333289 | CK605999 | 443 | 51 | 62 | 82 | 5e−23 | |
| 8421 | 2.084932276 | down | 0.0000317 | 0.000309767 | AI494682 | 344 | 47 | 104 | 45 | 1e−20 | |
| 8422 | 2.084806766 | down | 0.000149264 | 0.000913361 | BG507797 | 414 | 18 | 53 | 33 | 1.7 | proton transport |
| 8423 | 2.08475261 | down | 0.0000535 | 0.000443318 | BE822823 | 668 | 18 | 53 | 33 | 1.5 | proteolysis and peptidolysis |
| 8424 | 2.084580824 | down | 0.0000939 | 0.000659287 | CA799171 | 750 | 88 | 144 | 61 | 1e−42 | |
| 8425 | 2.08448636 | down | 0.0000294 | 0.000293392 | CD413457 | 721 | 153 | 192 | 79 | 2e−86 | protein biosynthesis |
| 8426 | 2.084484952 | down | 0.000100302 | 0.00068936 | AW831457 | 242 | 58 | 66 | 87 | 7e−30 | |
| 8427 | 2.084419582 | down | 0.00000928 | 0.000132783 | BI967222 | 501 | 33 | 93 | 35 | 1e−07 | cell fate specification |
| 8428 | 2.084305615 | down | 0.00086345 | 0.003277417 | BE806387 | 586 | 100 | 156 | 64 | 3e−50 | |
| 8429 | 2.084019893 | down | 0.00000747 | 0.000115956 | BI968713 | 800 | 102 | 206 | 49 | 3e−45 | |
| 8430 | 2.083920268 | down | 0.000155282 | 0.000940697 | BM092071 | 570 | 46 | 59 | 77 | 5e−20 | |
| 8431 | 2.083618623 | down | 0.0000136 | 0.000174035 | AI856531 | 292 | 12 | 30 | 40 | 2.4 | glycyl-tRNA aminoacylation |
| 8432 | 2.083530532 | down | 0.0000126 | 0.000164678 | BI971027 | 794 | 41 | 123 | 33 | 1e−08 | regulation of transcription\, DNA- |
| | | | | | | | | | | | dependent |
| 8433 | 2.08351283 | down | 0.000105852 | 0.000715458 | BI971363 | 630 | 64 | 151 | 42 | 2e−19 | |
| 8434 | 2.082857434 | down | 0.0000176 | 0.000205986 | AW349351 | 747 | 99 | 143 | 69 | 9e−53 | galactose metabolism |
| 8435 | 2.082640749 | down | 0.0000878 | 0.000628608 | AI736013 | 793 | 164 | 214 | 76 | 3e−93 | protein amino acid phosphorylation |
| 8436 | 2.082474573 | down | 0.0000183 | 0.000211148 | BU763133 | 569 | 16 | 47 | 34 | 0.67 | |
| 8437 | 2.081409151 | down | 0.00000924 | 0.000132463 | CD402683 | 454 | 15 | 39 | 38 | 2.7 | |
| 8438 | 2.081353285 | down | 0.000215586 | 0.00119198 | AF135862.1 | | | | | | |
| 8439 | 2.0397923 | down | 0.0000194 | 0.00022012 | BE658683 | | | | | | |
| 8440 | 2.039416181 | down | 0.0000159 | 0.000193214 | BE820647 | 716 | 59 | 104 | 56 | 2e−30 | |
| 8441 | 2.039389184 | down | 0.00106874 | 0.003826345 | BM892707 | 565 | 44 | 66 | 66 | 3e−20 | |
| 8442 | 2.039317854 | down | 0.0000299 | 0.000297021 | AW307100 | | | | | | |
| 8443 | 2.039305785 | down | 0.000336124 | 0.001644505 | BG047041 | 464 | 98 | 161 | 60 | 2e−54 | |
| 8444 | 2.038968317 | down | 0.000125488 | 0.00081199 | AW351166 | 362 | 12 | 28 | 42 | 4.6 | peroxisome organization and biogenesis |
| 8445 | 2.038920612 | down | 0.0000942 | 0.000660695 | BQ628012 | 339 | 26 | 92 | 28 | 1.4 | |
| 8446 | 2.038897216 | down | 0.000146382 | 0.000901148 | CF806471 | 512 | 105 | 168 | 62 | 9e−55 | fucose metabolism |
| 8447 | 2.038703857 | down | 0.0000284 | 0.000286559 | AW761338 | 465 | 115 | 153 | 75 | 6e−64 | |
| 8448 | 2.038479715 | down | 0.0000606 | 0.000483472 | CA799900 | 609 | 10 | 16 | 62 | 6.3 | protein amino acid phosphorylation |
| 8449 | 2.038096893 | down | 0.0000449 | 0.000393419 | BI973929 | 555 | 57 | 89 | 64 | 2e−24 | |
| 8450 | 2.038042958 | down | 0.000260502 | 0.001368317 | CK606070 | 258 | 48 | 85 | 56 | 1e−22 | electron transport |
| 8451 | 2.037817831 | down | 0.000313691 | 0.001568623 | BI945497 | 734 | 155 | 237 | 65 | 8e−83 | GMP biosynthesis |
| 8452 | 2.037520888 | down | 0.0000398 | 0.000360942 | BF597369 | 437 | 10 | 19 | 52 | 2.6 | electron transport |
| 8453 | 2.037290056 | down | 0.0000159 | 0.000193214 | BE822181 | 530 | 21 | 71 | 29 | 0.26 | |
| 8454 | 2.036816136 | down | 0.000111871 | 0.000744423 | CA936292 | 538 | 104 | 161 | 64 | 2e−51 | |
| 8455 | 2.036741336 | down | 0.0000461 | 0.000401182 | BQ079657 | 409 | 17 | 22 | 77 | 2e−04 | proteolysis and peptidolysis |
| 8456 | 2.036696281 | down | 0.0000821 | 0.000599045 | BU549426 | 530 | 60 | 118 | 50 | 1e−26 | antibiotic biosynthesis |
| 8457 | 2.03666661 | down | 0.0000199 | 0.000223191 | CA935386 | 332 | 8 | 16 | 50 | 9.5 | |
| 8458 | 2.036395389 | down | 0.0000141 | 0.00017769 | BQ453808 | 530 | 53 | 77 | 68 | 6e−27 | lipid metabolism |
| 8459 | 2.036256633 | down | 0.000118633 | 0.000777943 | BU091560 | 459 | 70 | 82 | 85 | 1e−33 | histidine biosynthesis |
| 8460 | 2.03620032 | down | 0.001559822 | 0.005019137 | AW755412 | 309 | 16 | 70 | 22 | 0.29 | |
| 8461 | 2.036184461 | down | 0.0000326 | 0.000315778 | BI320905 | 552 | 106 | 188 | 56 | 3e−56 | |
| 8462 | 2.036147226 | down | 0.0000609 | 0.000485138 | BU084135 | 515 | 78 | 102 | 76 | 1e−42 | metabolism |
| 8463 | 2.036044054 | down | 0.000155643 | 0.00094249 | BG044720 | 405 | 39 | 117 | 33 | 2e−08 | cell wall catabolism |
| 8464 | 2.03589915 | down | 0.0000117 | 0.000155816 | BE823449 | 715 | 99 | 186 | 53 | 2e−48 | heme biosynthesis |
| 8465 | 2.035804612 | down | 0.0000315 | 0.000308381 | CD409151 | 648 | 94 | 98 | 95 | 4e−50 | |
| 8466 | 2.035803764 | down | 0.0000841 | 0.000608867 | BG511605 | 255 | 9 | 17 | 52 | 4.2 | |
| 8467 | 2.035707941 | down | 0.000223068 | 0.001221321 | CF806975 | 412 | 124 | 135 | 91 | 1e−75 | |
| 8468 | 2.035546604 | down | 0.000100822 | 0.000691755 | BE609485 | 579 | 83 | 191 | 43 | 5e−41 | glycolysis |
| 8469 | 2.08082378 | down | 0.000439208 | 0.001998592 | AW133007 | 515 | 103 | 156 | 66 | 4e−48 | regulation of transcription\, DNA- |
| | | | | | | | | | | | dependent |
| 8470 | 2.080718047 | down | 0.00025351 | 0.001341076 | BQ741444 | 434 | 71 | 111 | 63 | 1e−32 | protein secretion |
| 8471 | 2.080469224 | down | 0.000115186 | 0.000760637 | BM523464 | 597 | 99 | 176 | 56 | 5e−60 | |
| 8472 | 2.080165154 | down | 0.000176049 | 0.001030324 | CF805944 | 684 | 142 | 226 | 62 | 4e−76 | |
| 8473 | 2.080111651 | down | 0.001208186 | 0.004170387 | CA935637 | 569 | 59 | 118 | 50 | 3e−21 | |
| 8474 | 2.079814118 | down | 0.000446733 | 0.002022882 | CD408001 | 294 | 20 | 46 | 43 | 3.3 | |
| 8475 | 2.079658915 | down | 0.00000907 | 0.000130834 | AW705887 | 611 | 56 | 175 | 32 | 4e−18 | antibiotic biosynthesis |
| 8476 | 2.079523359 | down | 0.000625921 | 0.002591439 | BM521156 | 547 | 117 | 188 | 62 | 3e−62 | |
| 8477 | 2.07934552 | down | 0.001042997 | 0.003758865 | AW133151 | 721 | 121 | 155 | 78 | 6e−64 | protein biosynthesis |
| 8478 | 2.079129098 | down | 0.0000227 | 0.000245182 | BU082845 | 598 | 57 | 165 | 34 | 3e−11 | |
| 8479 | 2.078643167 | down | 0.0000385 | 0.000352764 | BE022504 | 372 | 10 | 24 | 41 | 1.00 | protein amino acid phosphorylation |
| 8480 | 2.078357222 | down | 0.000100256 | 0.00068936 | BI498775 | 569 | 124 | 146 | 84 | 1e−72 | |
| 8481 | 2.078299705 | down | 0.000186164 | 0.001070371 | AW596835 | 541 | 137 | 180 | 76 | 2e−74 | ciliary/flagellar motility |
| 8482 | 2.078209869 | down | 0.000079 | 0.000582424 | CF807857 | 614 | 162 | 183 | 88 | 5e−94 | |
| 8483 | 2.077900175 | down | 0.00000785 | 0.000119244 | BF070413 | 391 | 89 | 122 | 72 | 1e−44 | |
| 8484 | 2.077216538 | down | 0.000108315 | 0.000727851 | BI945664 | 643 | 14 | 32 | 43 | 3.1 | protein amino acid prenylation |
| 8485 | 2.076980903 | down | 0.0000212 | 0.000233323 | CD397798 | 449 | 39 | 93 | 41 | 1e−13 | |
| 8486 | 2.076790756 | down | 0.000613129 | 0.002550002 | BM886154 | 581 | 113 | 194 | 58 | 3e−57 | |
| 8487 | 2.076619177 | down | 0.0000852 | 0.000614818 | AW308988 | 345 | 30 | 36 | 83 | 1e−13 | phosphate transport |
| 8488 | 2.076193882 | down | 0.0000659 | 0.000512768 | CD392633 | 696 | 37 | 67 | 55 | 2e−11 | |
| 8489 | 2.076047385 | down | 0.000252075 | 0.001335616 | CF805749 | 556 | 52 | 102 | 50 | 3e−18 | mitochondrial electron transport\, NADH |
| | | | | | | | | | | | to ubiquinone |
| 8490 | 2.075831743 | down | 0.0000775 | 0.000574101 | BU551219 | 682 | 69 | 138 | 50 | 5e−30 | |
| 8491 | 2.075761661 | down | 0.0000924 | 0.000651254 | AW704275 | 461 | 48 | 67 | 71 | 5e−23 | |
| 8492 | 2.075701989 | down | 0.0000496 | 0.000420793 | BE822862 | 763 | 54 | 125 | 43 | 4e−21 | 2′-hydroxy isoflavone/dihydroflavonol |
| | | | | | | | | | | | reductase homolog |
| 8493 | 2.07540102 | down | 0.0000127 | 0.000165664 | CF805939 | 460 | 44 | 64 | 68 | 4e−20 | |
| 8494 | 2.075325241 | down | 0.000876404 | 0.00331607 | BM523065 | 586 | 99 | 157 | 63 | 2e−52 | electron transport |
| 8495 | 2.07512162 | down | 0.0000596 | 0.000477916 | BQ629434 | 625 | 24 | 24 | 100 | 2e−06 | |
| 8496 | 2.075036662 | down | 0.00000588 | 0.000100049 | AW277394 | 454 | 16 | 50 | 32 | 4.7 | electron transport |
| 8497 | 2.074921454 | down | 0.00012698 | 0.000817791 | BU083431 | 534 | 59 | 80 | 73 | 5e−34 | |
| 8498 | 2.074672478 | down | 0.000104031 | 0.000707596 | CF806525 | | | | | | |
| 8499 | 2.074624765 | down | 0.0000114 | 0.000153343 | CD403227 | 465 | 73 | 91 | 80 | 3e−38 | transport |
| 8500 | 2.07461863 | down | 0.0000595 | 0.000477051 | CA938880 | 444 | 30 | 69 | 43 | 3e−04 | |
| 8501 | 2.074542819 | down | 0.0000308 | 0.000303081 | CD414912 | 532 | 21 | 27 | 77 | 1e−07 | regulation of transcription |
| 8502 | 2.074351821 | down | 0.0000257 | 0.000267985 | CF921971 | 613 | 92 | 135 | 68 | 3e−53 | protein modification |
| 8503 | 2.074296642 | down | 0.0000112 | 0.000151895 | BM525462 | 552 | 58 | 85 | 68 | 1e−26 | biosynthesis |
| 8504 | 2.071705007 | down | 0.000260154 | 0.001367254 | CF809006 | 556 | 140 | 164 | 85 | 2e−81 | protein biosynthesis |
| 8505 | 2.071466551 | down | 0.0000777 | 0.000574861 | BE612054 | 601 | 91 | 125 | 72 | 3e−49 | DNA repair |
| 8506 | 2.071346325 | down | 0.0000141 | 0.000177879 | BI892928 | 214 | 10 | 31 | 32 | 1.9 | protein amino acid phosphorylation |
| 8507 | 2.071136595 | down | 0.0000244 | 0.000258077 | CF807445 | 725 | 159 | 226 | 70 | 1e−88 | ATP synthase subunit |
| 8508 | 2.07088596 | down | 0.000082 | 0.000598808 | BE475334 | 430 | 125 | 142 | 88 | 3e−71 | |
| 8509 | 2.070818453 | down | 0.00000872 | 0.000127346 | CA784404 | 693 | 157 | 214 | 73 | 5e−85 | |
| 8510 | 2.070644483 | down | 0.0000142 | 0.000178833 | CD405808 | 684 | 67 | 180 | 37 | 5e−23 | regulation of transcription |
| 8511 | 2.070561165 | down | 0.000922001 | 0.003444068 | BE020433 | 410 | 80 | 128 | 62 | 5e−32 | extracellular transport |
| 8512 | 2.070297703 | down | 0.000300087 | 0.001518971 | AW185133 | 523 | 78 | 130 | 60 | 5e−35 | protein amino acid phosphorylation |
| 8513 | 2.070239851 | down | 0.000371164 | 0.001768552 | BI974279 | 542 | 77 | 122 | 63 | 1e−38 | regulation of transcription |
| 8514 | 2.070027723 | down | 0.00020757 | 0.001157875 | AI416735 | 485 | 17 | 46 | 36 | 3.2 | GAI1 |
| 8515 | 2.069845005 | down | 0.000133631 | 0.000847205 | BE660849 | 708 | 91 | 196 | 46 | 3e−45 | |
| 8516 | 2.069731945 | down | 0.0000391 | 0.000356022 | BM270428 | 310 | 26 | 34 | 76 | 2e−10 | carbohydrate metabolism |
| 8517 | 2.069629484 | down | 0.000301958 | 0.001525156 | BE610587 | 582 | 64 | 150 | 42 | 4e−20 | |
| 8518 | 2.069539082 | down | 0.0000152 | 0.000188013 | CD406689 | 522 | 66 | 75 | 88 | 3e−38 | electron transport |
| 8519 | 2.069453992 | down | 0.000202153 | 0.001136823 | BM143113 | 556 | 124 | 185 | 67 | 3e−69 | |
| 8520 | 2.069361352 | down | 0.0000264 | 0.000272756 | CD403850 | 556 | 58 | 92 | 63 | 2e−25 | |
| 8521 | 2.069275717 | down | 0.0000878 | 0.000628681 | AW317220 | 656 | 123 | 179 | 68 | 2e−68 | |
| 8522 | 2.069034387 | down | 0.0000399 | 0.000361016 | AI961002 | 450 | 54 | 125 | 43 | 1e−24 | |
| 8523 | 2.0688791 | down | 0.0000592 | 0.000476268 | BQ080586 | 548 | 39 | 79 | 49 | 2e−12 | |
| 8524 | 2.068738015 | down | 0.0000156 | 0.000190362 | BE658355 | 481 | 31 | 59 | 52 | 1e−10 | oligopeptide transport |
| 8525 | 2.068686409 | down | 0.0000725 | 0.000547634 | CA820222 | 534 | 18 | 46 | 39 | 3.8 | |
| 8526 | 2.068555564 | down | 0.000168253 | 0.000997269 | BG404904 | 326 | 41 | 82 | 50 | 1e−20 | electron transport |
| 8527 | 2.068305066 | down | 0.0000095 | 0.00013525 | BQ612798 | 570 | 41 | 147 | 27 | 5e−07 | |
| 8528 | 2.068291221 | down | 0.000137318 | 0.000862858 | CD398452 | 508 | 84 | 90 | 93 | 2e−44 | |
| 8529 | 2.068138922 | down | 0.0000519 | 0.000434029 | BI972871 | 353 | 76 | 113 | 67 | 7e−40 | |
| 8530 | 2.06813809 | down | 0.0000289 | 0.000289882 | BI970966 | 748 | 110 | 171 | 64 | 4e−63 | |
| 8531 | 2.068053532 | down | 0.0000996 | 0.000685855 | BG405055 | 496 | 14 | 45 | 31 | 7.4 | Choline kinase GmCK1p, L-serine |
| | | | | | | | | | | | biosynthesis |
| 8532 | 2.067709148 | down | 0.0000229 | 0.000246847 | CD409001 | 380 | 18 | 50 | 36 | 1.1 | transport |
| 8533 | 2.067572173 | down | 0.00022204 | 0.001217113 | CF922021 | 674 | 83 | 120 | 69 | 3e−46 | |
| 8534 | 2.067424152 | down | 0.0000411 | 0.000369544 | CK605778 | 734 | 86 | 175 | 49 | 6e−43 | Vegetative storage protien mRNA |
| | | | | | | | | | | | (VSP27) |
| 8535 | 2.067169856 | down | 0.0000351 | 0.00033256 | CA820555 | 428 | 15 | 29 | 51 | 0.034 | |
| 8536 | 2.06701482 | down | 0.000246646 | 0.001315561 | BM309127 | 565 | 66 | 76 | 86 | 4e−34 | |
| 8537 | 2.06681954 | down | 0.0000373 | 0.000345182 | CA819871 | 506 | 77 | 113 | 68 | 5e−41 | |
| 8538 | 2.066367804 | down | 0.0000295 | 0.000293594 | BE805798 | 370 | 79 | 121 | 65 | 8e−43 | protein amino acid phosphorylation |
| 8539 | 2.066320433 | down | 0.0000162 | 0.000195745 | AW432771 | 313 | 12 | 18 | 66 | 3.2 | pyridoxine biosynthesis |
| 8540 | 2.066234691 | down | 0.0000983 | 0.000679951 | BQ079384 | 522 | 86 | 178 | 48 | 6e−41 | electron transport |
| 8541 | 2.065953561 | down | 0.0000141 | 0.000177842 | BU544088 | 561 | 56 | 107 | 52 | 2e−23 | protein amino acid phosphorylation |
| 8542 | 2.065935759 | down | 0.001340141 | 0.004488248 | BG839173 | 823 | 34 | 99 | 34 | 7e−04 | |
| 8543 | 2.06587755 | down | 0.000528862 | 0.002292568 | AI443549 | 559 | 19 | 86 | 22 | 0.38 | cation transport |
| 8544 | 2.065665992 | down | 0.000225488 | 0.001230454 | CD397282 | 592 | 95 | 105 | 90 | 3e−54 | protein biosynthesis |
| 8545 | 2.065115748 | down | 0.0000148 | 0.000183966 | CD396464 | 731 | 118 | 169 | 69 | 2e−63 | acetyl-CoA metabolism |
| 8546 | 2.064984923 | down | 0.00100855 | 0.003670094 | BQ299346 | 420 | 94 | 139 | 67 | 9e−53 | proteolysis and peptidolysis |
| 8547 | 2.064672647 | down | 0.000178591 | 0.001038415 | AI431212 | 308 | 69 | 99 | 69 | 7e−35 | |
| 8548 | 2.06461336 | down | 0.0000241 | 0.000256374 | BQ612084 | | | | | | |
| 8549 | 2.063867255 | down | 0.000563338 | 0.002404607 | BM891639 | 562 | 12 | 39 | 30 | 1.9 | |
| 8550 | 2.063837171 | down | 0.0000353 | 0.000333289 | BE823496 | 665 | 92 | 164 | 56 | 4e−52 | |
| 8551 | 2.063829103 | down | 0.0000766 | 0.000569861 | AI900301 | 401 | 48 | 70 | 68 | 3e−22 | |
| 8552 | 2.063809844 | down | 0.0000484 | 0.000413782 | BU550458 | 627 | 57 | 73 | 78 | 7e−29 | |
| 8553 | 2.063773438 | down | 0.000290832 | 0.00148431 | BI699341 | 570 | 77 | 87 | 88 | 9e−38 | |
| 8554 | 2.063683631 | down | 0.00000838 | 0.000124012 | CF806245 | 660 | 47 | 113 | 41 | 5e−19 | protein amino acid phosphorylation |
| 8555 | 2.063433966 | down | 0.0000451 | 0.000395098 | AW351191 | 371 | 66 | 90 | 73 | 3e−36 | response to UV |
| 8556 | 2.0630924 | down | 0.0000609 | 0.00048534 | BG041928 | 506 | 100 | 108 | 92 | 1e−54 | |
| 8557 | 2.06209834 | down | 0.002102108 | 0.006200324 | BG044510 | 681 | 59 | 171 | 34 | 1e−18 | |
| 8558 | 2.0619452 | down | 0.0000275 | 0.000280621 | CA819048 | 363 | 10 | 25 | 40 | 3.5 | |
| 8559 | 2.061546942 | down | 0.0000411 | 0.000369301 | AW348087 | 672 | 99 | 155 | 63 | 2e−49 | |
| 8560 | 2.060649775 | down | 0.0000753 | 0.000563336 | AW306814 | 309 | 20 | 58 | 34 | 0.38 | |
| 8561 | 2.060633371 | down | 0.0000582 | 0.000470354 | BE607988 | 476 | 98 | 156 | 62 | 4e−46 | protein amino acid phosphorylation |
| 8562 | 2.060574605 | down | 0.000266293 | 0.001390387 | BM568090 | 529 | 42 | 63 | 66 | 2e−18 | |
| 8563 | 2.060425903 | down | 0.000017 | 0.000201703 | AW397102 | 341 | 43 | 93 | 46 | 2e−19 | |
| 8564 | 2.059997063 | down | 0.0000143 | 0.000179881 | BE820502 | 628 | 76 | 119 | 63 | 5e−35 | |
| 8565 | 2.059960189 | down | 0.0000372 | 0.000345061 | AW706581 | 457 | 54 | 71 | 76 | 4e−26 | |
| 8566 | 2.059933127 | down | 0.0000597 | 0.000478496 | BG650433 | 358 | 35 | 73 | 47 | 6e−13 | regulation of transcription\, DNA- |
| | | | | | | | | | | | dependent |
| 8567 | 2.059917382 | down | 0.0000478 | 0.000411119 | BQ253089 | 421 | 26 | 39 | 66 | 7e−10 | protein amino acid phosphorylation |
| 8568 | 2.059824413 | down | 0.00013616 | 0.000857448 | CF807792 | 604 | 102 | 136 | 75 | 9e−55 | Syringolide-induced protein 14-1-1, |
| | | | | | | | | | | | protein biosynthesis |
| 8569 | 2.059812898 | down | 0.0000435 | 0.000385626 | BI425364 | | | | | | |
| 8570 | 2.059714673 | down | 0.000120887 | 0.000789004 | BQ629471 | 538 | 138 | 177 | 77 | 2e−78 | signal transduction |
| 8571 | 2.059467118 | down | 0.000249747 | 0.001326467 | BI973119 | 553 | 73 | 102 | 71 | 8e−38 | |
| 8572 | 2.058987994 | down | 0.0000101 | 0.000141229 | AW471840 | 270 | 14 | 44 | 31 | 1.4 | tRNA processing |
| 8573 | 2.058980044 | down | 0.0000218 | 0.000238371 | CF808365 | 666 | 162 | 222 | 72 | 8e−87 | |
| 8574 | 2.058912738 | down | 0.0000335 | 0.000322518 | BM086662 | 542 | 71 | 137 | 51 | 1e−37 | electron transport |
| 8575 | 2.058866315 | down | 0.000142914 | 0.00088786 | BE329716 | 522 | 112 | 167 | 67 | 4e−60 | |
| 8576 | 2.058551965 | down | 0.0000351 | 0.00033256 | CA800208 | 668 | 131 | 227 | 57 | 3e−71 | |
| 8577 | 2.058433525 | down | 0.0000676 | 0.000522289 | CD418088 | 684 | 61 | 80 | 76 | 3e−27 | cell differentiation |
| 8578 | 2.058406316 | down | 0.0000974 | 0.000676746 | BE191495 | 639 | 113 | 210 | 53 | 1e−62 | electron transport |
| 8579 | 2.058383864 | down | 0.000101183 | 0.000692862 | CA785469 | 666 | 29 | 77 | 37 | 0.079 | flower development |
| 8580 | 2.058242947 | down | 0.000626647 | 0.002593063 | CD406755 | 704 | 26 | 97 | 26 | 0.95 | |
| 8581 | 2.058139022 | down | 0.000651239 | 0.002666317 | BG405007 | 294 | 71 | 94 | 75 | 2e−39 | tRNA aminoacylation for protein |
| | | | | | | | | | | | translation |
| 8582 | 2.058038413 | down | 0.0000966 | 0.000673366 | CD404946 | 353 | 90 | 118 | 76 | 2e−50 | |
| 8583 | 2.057963437 | down | 0.0000691 | 0.000531009 | AW705318 | 514 | 12 | 41 | 29 | 3.5 | |
| 8584 | 2.057938053 | down | 0.00018646 | 0.001071585 | BI425772 | 421 | 79 | 134 | 58 | 4e−42 | proteolysis and peptidolysis |
| 8585 | 2.057795233 | down | 0.0000349 | 0.000331189 | BI787878 | 497 | 51 | 67 | 76 | 1e−25 | |
| 8586 | 2.057528288 | down | 0.000778826 | 0.003032865 | BM520077 | 559 | 81 | 82 | 98 | 1e−44 | D-xylose metabolism |
| 8587 | 2.056778617 | down | 0.000440954 | 0.00200484 | AW705786 | 584 | 17 | 48 | 35 | 0.92 | |
| 8588 | 2.056379581 | down | 0.00119461 | 0.004134909 | CF805996 | 719 | 109 | 237 | 45 | 3e−40 | |
| 8589 | 2.056183589 | down | 0.000065 | 0.000508477 | AW164508 | 501 | 94 | 123 | 76 | 1e−54 | |
| 8590 | 2.056013232 | down | 0.0000282 | 0.000285584 | AW099840 | 460 | 53 | 77 | 68 | 4e−27 | carbohydrate metabolism |
| 8591 | 2.055964093 | down | 0.0000133 | 0.000170568 | CF808122 | 533 | 14 | 41 | 34 | 1.7 | histidine biosynthesis |
| 8592 | 2.055833176 | down | 0.000054 | 0.000446238 | AI856323 | 571 | 76 | 101 | 75 | 5e−42 | |
| 8593 | 2.055659914 | down | 0.0000392 | 0.000357165 | AW780768 | 438 | 89 | 119 | 74 | 2e−48 | electron transport |
| 8594 | 2.05535915 | down | 0.000391789 | 0.001841323 | BG507757 | 474 | 142 | 158 | 89 | 1e−81 | |
| 8595 | 2.055253676 | down | 0.0000193 | 0.00021876 | BQ630108 | 370 | 21 | 26 | 80 | 2e−06 | |
| 8596 | 2.055053997 | down | 0.0000563 | 0.00046021 | CD394203 | 321 | 25 | 43 | 58 | 5e−09 | |
| 8597 | 2.054749532 | down | 0.0000204 | 0.000227142 | BQ610957 | 603 | 121 | 208 | 58 | 3e−70 | proteolysis and peptidolysis |
| 8598 | 2.054670895 | down | 0.0000159 | 0.000193214 | BU548129 | 466 | 22 | 49 | 44 | 3e−07 | |
| 8599 | 2.054520647 | down | 0.000174145 | 0.001023107 | AW350315 | 754 | 77 | 164 | 46 | 9e−37 | response to brassinosteroid stimulus |
| 8600 | 2.054476494 | down | 0.0000339 | 0.00032423 | AW100747 | 194 | 14 | 31 | 45 | 3.2 | |
| 8601 | 2.054450841 | down | 0.000024 | 0.000255441 | BM091766 | 525 | 145 | 173 | 83 | 6e−80 | small GTPase mediated signal |
| | | | | | | | | | | | transduction |
| 8602 | 2.054417539 | down | 0.0000182 | 0.000210635 | CF806227 | 584 | 87 | 102 | 85 | 4e−50 | |
| 8603 | 2.054106918 | down | 0.0000369 | 0.000343217 | BQ610490 | 331 | 9 | 20 | 45 | 1.5 | response to pathogenic fungi |
| | | | | | | | | | | | (incompatible interaction) |
| 8604 | 2.053722547 | down | 0.0000517 | 0.000432787 | BI968045 | 724 | 137 | 207 | 66 | 2e−66 | |
| 8605 | 2.053548224 | down | 0.001791542 | 0.005535386 | BM886527 | 560 | 71 | 111 | 63 | 1e−37 | electron transport |
| 8606 | 2.053509977 | down | 0.000463296 | 0.002078283 | BI785381 | 551 | 84 | 184 | 45 | 5e−38 | |
| 8607 | 2.053043017 | down | 0.000017 | 0.000201315 | CA935501 | 480 | 30 | 41 | 73 | 5e−11 | metabolism |
| 8608 | 2.052399682 | down | 0.000413142 | 0.001913075 | BQ080389 | 545 | 129 | 165 | 78 | 2e−75 | signal transduction |
| 8609 | 2.052274099 | down | 0.000156944 | 0.00094771 | BE330849 | 667 | 140 | 221 | 63 | 3e−85 | proline biosynthesis |
| 8610 | 2.051873859 | down | 0.0000144 | 0.000180668 | CF806075 | 560 | 86 | 119 | 72 | 1e−51 | chitin catabolism |
| 8611 | 2.051873092 | down | 0.0000707 | 0.000539414 | BG156147 | 469 | 104 | 148 | 70 | 2e−56 | response to oxidative stress |
| 8612 | 2.051801076 | down | 0.0000251 | 0.000264072 | BU544247 | 510 | 88 | 132 | 66 | 3e−41 | |
| 8613 | 2.051797218 | down | 0.0000943 | 0.000660989 | AW309726 | 470 | 16 | 55 | 29 | 1.3 | glycolysis |
| 8614 | 2.051626115 | down | 0.000237483 | 0.00127902 | BE660364 | 659 | 125 | 194 | 64 | 2e−67 | protein folding |
| 8615 | 2.051619721 | down | 0.000493482 | 0.002177026 | BI971415 | 689 | 106 | 212 | 50 | 3e−60 | electron transport |
| 8616 | 2.051590432 | down | 0.000146919 | 0.000903125 | BI968817 | 629 | 89 | 188 | 47 | 8e−36 | |
| 8617 | 2.05152391 | down | 0.000256667 | 0.001354224 | BM954372 | 404 | 45 | 82 | 54 | 2e−19 | protein amino acid phosphorylation |
| 8618 | 2.051509489 | down | 0.000109895 | 0.00073525 | CA819733 | 395 | 17 | 53 | 32 | 0.68 | proteolysis and peptidolysis |
| 8619 | 2.05133236 | down | 0.001662451 | 0.005258277 | CA936700 | 449 | 84 | 123 | 68 | 5e−42 | ‘de novo’ pyrimidine base biosynthesis |
| 8620 | 2.051281572 | down | 0.0000133 | 0.000170634 | BI785707 | 591 | 135 | 176 | 76 | 1e−73 | |
| 8621 | 2.051253901 | down | 0.0000337 | 0.000323572 | CA800345 | 243 | 13 | 25 | 52 | 0.83 | |
| 8622 | 2.050756474 | down | 0.0000913 | 0.000646624 | AW184809 | 570 | 62 | 151 | 41 | 4e−19 | |
| 8623 | 2.050702654 | down | 0.000251288 | 0.001332387 | AI965691 | 470 | 93 | 129 | 72 | 3e−55 | |
| 8624 | 2.050579917 | down | 0.0000558 | 0.000457265 | AI495029 | 450 | 27 | 75 | 36 | 3e−05 | |
| 8625 | 2.050573956 | down | 0.0000354 | 0.000333752 | BI497812 | 423 | 12 | 37 | 32 | 2.3 | |
| 8626 | 2.050469636 | down | 0.0000155 | 0.000189782 | CF807823 | 746 | 18 | 48 | 37 | 0.16 | |
| 8627 | 2.050033448 | down | 0.00069078 | 0.002777743 | CA801961 | 453 | 124 | 142 | 87 | 8e−72 | DNA replication initiation |
| 8628 | 2.049062026 | down | 0.00014251 | 0.000886159 | BQ628103 | 274 | 12 | 32 | 37 | 1.9 | |
| 8629 | 2.049053558 | down | 0.0000683 | 0.000526474 | BM307907 | 577 | 90 | 174 | 51 | 4e−42 | metal ion transport |
| 8630 | 2.048954646 | down | 0.0000787 | 0.000580732 | CA783832 | 799 | 173 | 186 | 93 | 2e−95 | Glyceraldehyde-3 phosphate |
| | | | | | | | | | | | dehydrogenase (GAPDH), response to |
| | | | | | | | | | | | stress |
| 8631 | 2.04895005 | down | 0.0000196 | 0.000221349 | BQ629141 | 436 | 88 | 105 | 83 | 1e−50 | cell growth and/or maintenance |
| 8632 | 2.048906566 | down | 0.0000451 | 0.000394673 | AW101443 | 416 | 69 | 116 | 59 | 1e−32 | |
| 8633 | 2.048885948 | down | 0.0000177 | 0.000207083 | CD414993 | 590 | 85 | 86 | 98 | 1e−41 | nucleosome assembly |
| 8634 | 2.04885171 | down | 0.000334284 | 0.001638274 | AW309517 | 636 | 51 | 80 | 63 | 1e−21 | intracellular protein transport |
| 8635 | 2.048843574 | down | 0.000204431 | 0.001146089 | BU544092 | 554 | 50 | 80 | 62 | 7e−21 | protein amino acid phosphorylation |
| 8636 | 2.048736432 | down | 0.001306423 | 0.004411508 | BM892454 | 534 | 117 | 182 | 64 | 5e−59 | |
| 8637 | 2.048630348 | down | 0.000211095 | 0.001173535 | BF597623 | 207 | 52 | 69 | 75 | 6e−28 | proteolysis and peptidolysis |
| 8638 | 2.047672534 | down | 0.0000174 | 0.000204667 | AW348614 | 719 | 82 | 169 | 48 | 1e−37 | metabolism |
| 8639 | 2.047603468 | down | 0.000734533 | 0.002902786 | BU578740 | 550 | 135 | 183 | 73 | 6e−78 | |
| 8640 | 2.047559988 | down | 0.001333053 | 0.004473722 | BE610220 | 340 | 59 | 112 | 52 | 9e−24 | transport |
| 8641 | 2.047525904 | down | 0.000204452 | 0.001146089 | BG650489 | 416 | 52 | 87 | 59 | 4e−22 | |
| 8642 | 2.047481276 | down | 0.0000138 | 0.000174871 | CF806738 | 701 | 96 | 212 | 45 | 1e−47 | |
| 8643 | 2.047255773 | down | 0.000366897 | 0.001753777 | BM309062 | 571 | 136 | 173 | 78 | 9e−78 | Glyceraldehyde-3 phosphate |
| | | | | | | | | | | | dehydrogenase (GAPDH) |
| 8644 | 2.046528926 | down | 0.0000631 | 0.000497577 | BI944491 | 699 | 192 | 212 | 90 | e−104 | |
| 8645 | 2.046156318 | down | 0.0000505 | 0.000425501 | BM178639 | 420 | 55 | 61 | 90 | 2e−31 | |
| 8646 | 2.046061306 | down | 0.000016 | 0.000193231 | BU084838 | 565 | 51 | 173 | 29 | 1e−16 | |
| 8647 | 2.045888915 | down | 0.0000404 | 0.000364405 | CF805761 | 544 | 48 | 83 | 57 | 8e−25 | Vegetative storage protien mRNA |
| | | | | | | | | | | | (VSP27), carbohydrate metabolism |
| 8648 | 2.045800049 | down | 0.000377966 | 0.001792508 | BG155341 | 344 | 98 | 110 | 89 | 8e−55 | carbohydrate biosynthesis |
| 8649 | 2.045360473 | down | 0.0000242 | 0.000256666 | CD394450 | 376 | 10 | 21 | 47 | 6.5 | |
| 8650 | 2.045332005 | down | 0.0000473 | 0.000408196 | BF598781 | 431 | 15 | 43 | 34 | 0.65 | |
| 8651 | 2.044541412 | down | 0.000113531 | 0.000752355 | BM954614 | 216 | 11 | 21 | 52 | 5.5 | protein neddylation |
| 8652 | 2.04452713 | down | 0.0000168 | 0.000199627 | CD398886 | 709 | 103 | 114 | 90 | 2e−57 | intracellular protein transport |
| 8653 | 2.044407406 | down | 0.000046 | 0.000400306 | CF808699 | 646 | 86 | 127 | 67 | 2e−44 | |
| 8654 | 2.044198014 | down | 0.000043 | 0.000382673 | BE659771 | 650 | 18 | 50 | 36 | 0.005 | |
| 8655 | 2.044148179 | down | 0.0000348 | 0.000330398 | AW349391 | 767 | 131 | 164 | 79 | 8e−70 | enolase, response to cold |
| 8656 | 2.044003113 | down | 0.00177457 | 0.005502321 | BG044815 | 419 | 54 | 134 | 40 | 8e−17 | response to auxin stimulus |
| 8657 | 2.043758762 | down | 0.0000422 | 0.000376502 | BM521435 | 544 | 93 | 117 | 79 | 6e−47 | |
| 8658 | 2.043616496 | down | 0.000210989 | 0.00117312 | BU761371 | 451 | 111 | 150 | 74 | 5e−62 | methylation-dependent chromatin |
| | | | | | | | | | | | silencing |
| 8659 | 2.043494827 | down | 0.0000668 | 0.000517819 | AW432391 | 382 | 14 | 37 | 37 | 0.36 | |
| 8660 | 2.04321219 | down | 0.0000845 | 0.000610794 | BU551211 | 672 | 122 | 176 | 69 | 1e−70 | signal transduction |
| 8661 | 2.043126463 | down | 0.000575503 | 0.00244405 | CA786021 | 507 | 57 | 140 | 40 | 1e−18 | regulation of transcription |
| 8662 | 2.043012551 | down | 0.0000291 | 0.000291205 | BI973175 | 316 | 10 | 26 | 38 | 0.38 | intracellular signaling cascade |
| 8663 | 2.042971507 | down | 0.0000442 | 0.00038939 | BG046959 | 464 | 93 | 153 | 60 | 3e−49 | electron transport |
| 8664 | 2.042897862 | down | 0.0000588 | 0.000474103 | BM085051 | 552 | 21 | 32 | 65 | 8e−07 | |
| 8665 | 2.042062065 | down | 0.001072513 | 0.003837232 | BE611850 | 701 | 51 | 84 | 60 | 5e−20 | protein amino acid phosphorylation |
| 8666 | 2.042007804 | down | 0.0000547 | 0.000450301 | CF805959 | 678 | 71 | 160 | 44 | 4e−30 | regulation of transcription |
| 8667 | 2.041535326 | down | 0.0000561 | 0.00045899 | BF595610 | 522 | 154 | 174 | 88 | 2e−90 | protein amino acid phosphorylation |
| 8668 | 2.041206592 | down | 0.000582034 | 0.002462634 | BM523917 | 545 | 102 | 153 | 66 | 1e−53 | |
| 8669 | 2.04112162 | down | 0.0000917 | 0.000647935 | CA938534 | 588 | 78 | 168 | 46 | 5e−31 | |
| 8670 | 2.041016399 | down | 0.0000735 | 0.000553611 | BE658965 | 421 | 20 | 53 | 37 | 5.1 | |
| 8671 | 2.040740176 | down | 0.000853762 | 0.003250948 | BG155821 | 694 | 182 | 230 | 79 | e−104 | amino acid metabolism |
| 8672 | 2.040674744 | down | 0.000129837 | 0.000830133 | CF807444 | 668 | 101 | 137 | 73 | 1e−58 | electron transport |
| 8673 | 2.040647018 | down | 0.000283517 | 0.001456846 | CF806140 | 682 | 116 | 207 | 56 | 2e−52 | regulation of transcription |
| 8674 | 2.040061672 | down | 0.000053 | 0.000440695 | BE330662 | 593 | 148 | 174 | 85 | 2e−85 | DNA replication |
| 8675 | 2.035377623 | down | 0.0000381 | 0.000351006 | BM527731 | 464 | 31 | 54 | 57 | 3e−12 | |
| 8676 | 2.035043297 | down | 0.0000376 | 0.00034733 | BE820074 | 499 | 34 | 53 | 64 | 5e−13 | protein amino acid phosphorylation |
| 8677 | 2.034993616 | down | 0.0000125 | 0.000163391 | BG047108 | 457 | 17 | 55 | 30 | 0.43 | |
| 8678 | 2.034640325 | down | 0.0000137 | 0.000174275 | BU926903 | 471 | 29 | 100 | 29 | 0.032 | |
| 8679 | 2.034403989 | down | 0.001066134 | 0.003819559 | CF805849 | 598 | 76 | 170 | 44 | 2e−33 | |
| 8680 | 2.03423142 | down | 0.000400657 | 0.001870695 | BI893334 | 506 | 94 | 128 | 73 | 4e−49 | metabolism |
| 8681 | 2.034228385 | down | 0.0000569 | 0.000464083 | BI784833 | 413 | 14 | 36 | 38 | 0.77 | |
| 8682 | 2.033937124 | down | 0.000013 | 0.000168352 | BU964784 | 456 | 90 | 147 | 61 | 1e−46 | |
| 8683 | 2.033736529 | down | 0.0000217 | 0.000237325 | BE347006 | 478 | 82 | 122 | 67 | 5e−49 | |
| 8684 | 2.033583569 | down | 0.000108406 | 0.000728268 | BE822926 | 649 | 64 | 68 | 94 | 2e−31 | glycolysis |
| 8685 | 2.033381178 | down | 0.000319546 | 0.00158887 | AW349120 | 670 | 59 | 132 | 44 | 4e−14 | regulation of transcription\, DNA- |
| | | | | | | | | | | | dependent |
| 8686 | 2.033370979 | down | 0.000149908 | 0.00091581 | BG047161 | 413 | 18 | 55 | 32 | 0.091 | photomorphogenesis |
| 8687 | 2.033195187 | down | 0.0000621 | 0.000492006 | AW350126 | 729 | 35 | 110 | 31 | 9e−05 | |
| 8688 | 2.033174501 | down | 0.000287721 | 0.001472627 | BM527377 | 558 | 77 | 113 | 68 | 3e−41 | |
| 8689 | 2.032928211 | down | 0.0000359 | 0.000337399 | CD406462 | 527 | 21 | 31 | 67 | 2e−07 | |
| 8690 | 2.032924243 | down | 0.000325495 | 0.001608838 | BM522856 | 405 | 10 | 34 | 29 | 2.1 | regulation of transcription\, DNA- |
| | | | | | | | | | | | dependent |
| 8691 | 2.032840479 | down | 0.000272271 | 0.001411799 | BU548983 | 668 | 72 | 90 | 80 | 6e−42 | carbohydrate metabolism |
| 8692 | 2.032657802 | down | 0.0000448 | 0.000393017 | BI470204 | 476 | 88 | 135 | 65 | 2e−43 | lipid metabolism |
| 8693 | 2.032588087 | down | 0.0000193 | 0.000218938 | AW348596 | 665 | 65 | 115 | 56 | 1e−30 | electron transport |
| 8694 | 2.032554698 | down | 0.0000539 | 0.00044576 | BG508205 | 312 | 36 | 41 | 87 | 3e−17 | alternative oxidase 2b |
| 8695 | 2.032250625 | down | 0.000361501 | 0.001734373 | AW509466 | 498 | 46 | 64 | 71 | 4e−22 | signal transduction |
| 8696 | 2.031950659 | down | 0.0000196 | 0.00022145 | BI320818 | 443 | 16 | 51 | 31 | 2.0 | regulation of transcription\, DNA- |
| | | | | | | | | | | | dependent |
| 8697 | 2.031813367 | down | 0.0000127 | 0.000165111 | AW201538 | 451 | 16 | 48 | 33 | 0.19 | protein biosynthesis |
| 8698 | 2.031452308 | down | 0.0000229 | 0.000247185 | BI970500 | 718 | 38 | 79 | 48 | 3e−16 | |
| 8699 | 2.031380784 | down | 0.0000309 | 0.000304355 | BI497609 | 462 | 69 | 148 | 46 | 7e−23 | ossification |
| 8700 | 2.031192346 | down | 0.0001031 | 0.000702411 | BG041461 | | | | | | |
| 8701 | 2.030753809 | down | 0.0000184 | 0.00021144 | CF807880 | 705 | 40 | 118 | 33 | 1e−06 | |
| 8702 | 2.030430297 | down | 0.0000654 | 0.000509651 | BF425263 | 422 | 67 | 141 | 47 | 3e−29 | regulation of transcription\, DNA- |
| | | | | | | | | | | | dependent |
| 8703 | 2.030297679 | down | 0.000139839 | 0.000873161 | BE821755 | 273 | 14 | 33 | 42 | 1.9 | |
| 8704 | 2.030068239 | down | 0.000156396 | 0.000945162 | BE346131 | 264 | 36 | 52 | 69 | 1e−13 | transport |
| 8705 | 2.029902015 | down | 0.0000185 | 0.00021253 | BQ628119 | 235 | 12 | 40 | 30 | 1.4 | |
| 8706 | 2.029519291 | down | 0.0000223 | 0.000242485 | BM176987 | 438 | 16 | 37 | 43 | 0.18 | amino acid transport |
| 8707 | 2.029410805 | down | 0.000149447 | 0.000914035 | BG154527 | 430 | 93 | 104 | 89 | 4e−46 | microtubule-based process |
| 8708 | 2.029408415 | down | 0.0000791 | 0.000582949 | BU551115 | 458 | 24 | 69 | 40 | 2e−05 | regulation of cell proliferation |
| 8709 | 2.02940702 | down | 0.000509645 | 0.002228008 | BG726755 | 470 | 77 | 151 | 50 | 6e−31 | |
| 8710 | 2.029056865 | down | 0.0000248 | 0.000261864 | CD401772 | 630 | 44 | 73 | 60 | 5e−19 | |
| 8711 | 2.028676621 | down | 0.0000232 | 0.000249423 | BU545778 | 511 | 36 | 98 | 36 | 2e−12 | protein amino acid phosphorylation |
| 8712 | 2.028576187 | down | 0.0000537 | 0.000444472 | BE346270 | 525 | 46 | 98 | 46 | 2e−23 | regulation of transcription |
| 8713 | 2.028272445 | down | 0.000580452 | 0.002459795 | BU090050 | 331 | 35 | 63 | 55 | 9e−16 | |
| 8714 | 2.027584459 | down | 0.000204017 | 0.001144162 | CF806279 | 651 | 184 | 214 | 85 | e−104 | |
| 8715 | 2.027311528 | down | 0.0000261 | 0.000270482 | CF808828 | 649 | 110 | 214 | 51 | 3e−63 | DNA repair |
| 8716 | 2.026949454 | down | 0.000338756 | 0.0016533 | CA820125 | 450 | 27 | 47 | 57 | 2e−07 | |
| 8717 | 2.026901834 | down | 0.000804313 | 0.003107696 | CF806485 | 212 | 12 | 26 | 46 | 3.3 | |
| 8718 | 2.026860069 | down | 0.000122686 | 0.000796974 | BI945671 | 371 | 13 | 38 | 34 | 2.9 | |
| 8719 | 2.026725177 | down | 0.0000258 | 0.000268374 | BQ473906 | 565 | 37 | 138 | 26 | 0.005 | |
| 8720 | 2.026713354 | down | 0.0000913 | 0.000646624 | BE021628 | 597 | 79 | 236 | 33 | 7e−23 | protein amino acid phosphorylation |
| 8721 | 2.026690567 | down | 0.0000696 | 0.000533204 | BE822297 | 591 | 112 | 177 | 63 | 5e−56 | |
| 8722 | 2.02663275 | down | 0.0000763 | 0.000567644 | CA820023 | 588 | 113 | 180 | 62 | 2e−56 | |
| 8723 | 2.026541059 | down | 0.000286661 | 0.001468 | BU080611 | 580 | 96 | 96 | 100 | 7e−51 | small GTPase mediated signal |
| | | | | | | | | | | | transduction |
| 8724 | 2.026540613 | down | 0.000467122 | 0.002091956 | CF808929 | 705 | 35 | 78 | 44 | 1e−09 | |
| 8725 | 2.026532733 | down | 0.0000244 | 0.000257893 | CD403241 | 365 | 14 | 33 | 42 | 0.42 | cytokinesis |
| 8726 | 2.025961184 | down | 0.0000251 | 0.000264072 | BU548967 | 557 | 37 | 85 | 43 | 2e−17 | proteolysis and peptidolysis |
| 8727 | 2.025620389 | down | 0.000187547 | 0.001076018 | BM270176 | 295 | 18 | 58 | 31 | 0.66 | |
| 8728 | 2.02533595 | down | 0.0000548 | 0.00045068 | BF598279 | 433 | 21 | 28 | 75 | 2e−07 | metabolism |
| 8729 | 2.025304079 | down | 0.000029 | 0.000290681 | BE555669 | 560 | 42 | 102 | 41 | 2e−20 | |
| 8730 | 2.025245564 | down | 0.000186743 | 0.001072391 | BU545140 | 405 | 14 | 14 | 100 | 0.15 | |
| 8731 | 2.025210306 | down | 0.000141373 | 0.00088069 | BI973015 | 588 | 81 | 103 | 78 | 3e−44 | protein amino acid phosphorylation |
| 8732 | 2.024822162 | down | 0.00000939 | 0.000133857 | CF808995 | 517 | 13 | 28 | 46 | 2.1 | |
| 8733 | 2.023884234 | down | 0.000135027 | 0.000852568 | AI856110 | 357 | 12 | 30 | 40 | 3.3 | |
| 8734 | 2.023855649 | down | 0.0000172 | 0.000202543 | BI968925 | 800 | 16 | 46 | 34 | 0.002 | ER to Golgi transport |
| 8735 | 2.023800446 | down | 0.000019 | 0.000217041 | BE474915 | 411 | 87 | 136 | 63 | 2e−43 | carbohydrate metabolism |
| 8736 | 2.023698497 | down | 0.00016315 | 0.000974534 | BU549333 | 643 | 76 | 186 | 40 | 3e−21 | regulation of transcription |
| 8737 | 2.023566056 | down | 0.000150695 | 0.000919719 | BQ133883 | 558 | 130 | 186 | 69 | 2e−68 | carbohydrate biosynthesis |
| 8738 | 2.02347143 | down | 0.0000439 | 0.000387914 | BQ629224 | 209 | 14 | 38 | 36 | 0.86 | metabolism |
| 8739 | 2.023190734 | down | 0.000037 | 0.00034356 | CD395820 | 508 | 44 | 55 | 80 | 3e−22 | |
| 8740 | 2.022917542 | down | 0.000108963 | 0.000730188 | CF807947 | 468 | 106 | 133 | 79 | 1e−60 | transport |
| 8741 | 2.022738344 | down | 0.000435512 | 0.001987544 | CA785213 | 721 | 62 | 82 | 75 | 4e−34 | |
| 8742 | 2.022450945 | down | 0.0000185 | 0.00021244 | AW310600 | 646 | 67 | 78 | 85 | 2e−35 | |
| 8743 | 2.022400571 | down | 0.0000364 | 0.000340199 | CD401201 | 722 | 19 | 30 | 63 | 5e−06 | DNA metabolism |
| 8744 | 2.022395719 | down | 0.0000128 | 0.000165801 | AB052788.1 | | | | | | nitrate transporter NRT1-5 |
| 8745 | 2.02196476 | down | 0.0000394 | 0.000358382 | BU546620 | 538 | 18 | 30 | 60 | 0.002 | |
| 8746 | 2.021573425 | down | 0.0000756 | 0.000564856 | CD415774 | 595 | 45 | 85 | 52 | 3e−14 | |
| 8747 | 2.021200116 | down | 0.0000767 | 0.000569861 | BE330493 | 632 | 40 | 52 | 76 | 2e−24 | |
| 8748 | 2.020992716 | down | 0.0000261 | 0.00027023 | BQ081272 | 552 | 154 | 182 | 84 | 9e−96 | carbohydrate biosynthesis |
| 8749 | 2.020933285 | down | 0.000146908 | 0.000903125 | CA936974 | 478 | 12 | 29 | 41 | 5.2 | |
| 8750 | 2.020741327 | down | 0.000176967 | 0.001033126 | BG881523 | 469 | 18 | 35 | 51 | 0.006 | regulation of transcription |
| 8751 | 2.020718325 | down | 0.000037 | 0.00034358 | BM178013 | 565 | 135 | 188 | 71 | 4e−73 | isoprenoid biosynthesis |
| 8752 | 2.020647035 | down | 0.000087 | 0.000624468 | BG651678 | 440 | 10 | 33 | 30 | 5.8 | G-protein coupled receptor protein |
| | | | | | | | | | | | signaling pathway |
| 8753 | 2.020643221 | down | 0.0000399 | 0.000361079 | CD402231 | 745 | 30 | 35 | 85 | 2e−14 | |
| 8754 | 2.020290376 | down | 0.00042674 | 0.001959162 | AW459328 | 596 | 39 | 83 | 46 | 3e−10 | regulation of transcription |
| 8755 | 2.020189291 | down | 0.000132384 | 0.000841842 | BE058403 | 243 | 12 | 37 | 32 | 0.37 | |
| 8756 | 2.020065388 | down | 0.000780268 | 0.003036941 | CF806314 | 700 | 130 | 227 | 57 | 3e−69 | amino acid transport |
| 8757 | 2.019935678 | down | 0.000301709 | 0.001524422 | BM093439 | 564 | 152 | 193 | 78 | 9e−78 | starch biosynthesis |
| 8758 | 2.019707754 | down | 0.0000545 | 0.000449309 | CD399549 | 671 | 84 | 128 | 65 | 9e−46 | protein amino acid phosphorylation |
| 8759 | 2.019322207 | down | 0.000128991 | 0.00082599 | BI970908 | 774 | 59 | 90 | 65 | 5e−30 | carbohydrate metabolism |
| 8760 | 2.019200575 | down | 0.0000693 | 0.000531633 | CF808264 | 562 | 124 | 187 | 66 | 3e−71 | protein amino acid phosphorylation |
| 8761 | 2.019069907 | down | 0.0000167 | 0.0001987 | CK606162 | 612 | 69 | 93 | 74 | 2e−36 | L-serine biosynthesis |
| 8762 | 2.018318988 | down | 0.0000592 | 0.000476067 | BM892048 | 468 | 44 | 47 | 93 | 4e−21 | isoprenoid biosynthesis |
| 8763 | 2.017995056 | down | 0.0000986 | 0.000680833 | BU761753 | 587 | 88 | 193 | 45 | 4e−42 | protein amino acid phosphorylation |
| 8764 | 2.017871082 | down | 0.000319965 | 0.001589219 | BI943988 | 304 | 74 | 75 | 98 | 3e−39 | |
| 8765 | 2.017739852 | down | 0.0000925 | 0.000651865 | CA819767 | 349 | 8 | 16 | 50 | 7.1 | |
| 8766 | 2.01748535 | down | 0.0000421 | 0.000375905 | BU549171 | 695 | 46 | 80 | 57 | 3e−18 | |
| 8767 | 2.01721966 | down | 0.0000137 | 0.000174412 | BI971676 | 490 | 17 | 34 | 50 | 0.005 | protein amino acid phosphorylation |
| 8768 | 2.017207996 | down | 0.0000433 | 0.000384639 | BM139929 | 322 | 15 | 50 | 30 | 0.099 | |
| 8769 | 2.017173965 | down | 0.0000825 | 0.000601261 | CF809176 | 548 | 158 | 182 | 86 | 4e−93 | tricarboxylic acid cycle |
| 8770 | 2.017109797 | down | 0.0000752 | 0.000562921 | CD410897 | 441 | 19 | 61 | 31 | 0.40 | |
| 8771 | 2.016824644 | down | 0.001278395 | 0.00434338 | AW507693 | 556 | 116 | 150 | 77 | 1e−69 | nitrogen metabolism |
| 8772 | 2.016798339 | down | 0.0000191 | 0.00021741 | BI788030 | 507 | 42 | 58 | 72 | 8e−18 | |
| 8773 | 2.016540963 | down | 0.000247445 | 0.0013187 | BF008815 | 629 | 138 | 208 | 66 | 5e−67 | |
| 8774 | 2.016537739 | down | 0.000109921 | 0.000735297 | BM732274 | 568 | 96 | 189 | 50 | 1e−52 | |
| 8775 | 2.016525217 | down | 0.0000153 | 0.000188261 | BG650418 | 287 | 17 | 59 | 28 | 3.2 | cell wall modification |
| 8776 | 2.016084295 | down | 0.000517453 | 0.002254326 | BM520077 | 559 | 81 | 82 | 98 | 1e−44 | D-xylose metabolism |
| 8777 | 2.016081475 | down | 0.001777436 | 0.00550803 | U68763.1 | | | | | | scof-1 protein |
| 8778 | 2.016056281 | down | 0.0000708 | 0.000539944 | BG044834 | 239 | 15 | 24 | 62 | 0.002 | regulation of transcription |
| 8779 | 2.01594099 | down | 0.000112038 | 0.000745217 | BG363130 | 522 | 22 | 75 | 29 | 0.11 | metabolism |
| 8780 | 2.015928821 | down | 0.000135049 | 0.000852568 | BE821063 | 611 | 12 | 33 | 36 | 1.3 | regulation of transcription |
| 8781 | 2.015829514 | down | 0.000087 | 0.000624443 | BU547775 | 634 | 96 | 111 | 86 | 6e−49 | |
| 8782 | 2.015763405 | down | 0.00067574 | 0.00273716 | BQ785421 | 591 | 159 | 190 | 83 | 1e−90 | |
| 8783 | 2.015487692 | down | 0.0000428 | 0.000381138 | BE820356 | 476 | 15 | 48 | 31 | 6.8 | |
| 8784 | 2.015420174 | down | 0.0000369 | 0.000343317 | AW102083 | 499 | 34 | 47 | 72 | 9e−14 | trehalose biosynthesis |
| 8785 | 2.015289869 | down | 0.000139315 | 0.000871331 | BM525617 | 427 | 15 | 28 | 53 | 0.21 | |
| 8786 | 2.015116616 | down | 0.0000237 | 0.000252555 | BG510658 | 307 | 11 | 25 | 44 | 4.2 | protein amino acid phosphorylation |
| 8787 | 2.01488382 | down | 0.000117122 | 0.000770117 | BM270616 | 554 | 72 | 87 | 82 | 7e−37 | electron transport |
| 8788 | 2.014791958 | down | 0.000252358 | 0.001336177 | BU548332 | 653 | 89 | 122 | 72 | 1e−41 | protein amino acid phosphorylation |
| 8789 | 2.014671783 | down | 0.000321111 | 0.001592387 | CK605547 | 686 | 115 | 189 | 60 | 6e−55 | L-serine biosynthesis |
| 8790 | 2.014397652 | down | 0.000049 | 0.000417179 | BI316563 | 414 | 12 | 36 | 33 | 3.8 | |
| 8791 | 2.014255705 | down | 0.0000682 | 0.000525366 | CD399794 | 581 | 84 | 119 | 70 | 4e−43 | |
| 8792 | 2.013552657 | down | 0.0000183 | 0.000210717 | CF806021 | 634 | 45 | 55 | 81 | 3e−20 | metabolism |
| 8793 | 2.013477802 | down | 0.0000601 | 0.000480712 | BU550919 | 709 | 37 | 43 | 86 | 3e−20 | |
| 8794 | 2.013471402 | down | 0.000125823 | 0.000813488 | BQ630441 | 612 | 45 | 62 | 72 | 2e−24 | |
| 8795 | 2.01343647 | down | 0.000720865 | 0.002863783 | BM525275 | 595 | 158 | 198 | 79 | 2e−95 | DNA replication initiation |
| 8796 | 2.013367776 | down | 0.0000228 | 0.000246214 | BM093425 | 543 | 87 | 128 | 67 | 3e−46 | |
| 8797 | 2.013271181 | down | 0.0000482 | 0.000413158 | BG156327 | 479 | 41 | 120 | 34 | 1e−09 | |
| 8798 | 2.012863132 | down | 0.0000724 | 0.000547208 | BU761623 | 471 | 34 | 89 | 38 | 4e−10 | regulation of transcription\, DNA- |
| | | | | | | | | | | | dependent |
| 8799 | 2.0125351 | down | 0.000153485 | 0.000931459 | AW348454 | 789 | 78 | 106 | 73 | 2e−45 | |
| 8800 | 2.012534639 | down | 0.0000465 | 0.00040343 | BI969757 | 786 | 118 | 128 | 92 | 5e−63 | transcription termination |
| 8801 | 2.012459783 | down | 0.0000499 | 0.000422085 | BU547464 | 669 | 122 | 169 | 72 | 1e−71 | carbohydrate metabolism |
| 8802 | 2.012377199 | down | 0.0000433 | 0.000384639 | BU763440 | 512 | 29 | 90 | 32 | 0.006 | protein biosynthesis |
| 8803 | 2.01228119 | down | 0.0000514 | 0.000431163 | CK606003 | 585 | 168 | 193 | 87 | 2e−93 | |
| 8804 | 2.012214641 | down | 0.000506388 | 0.002217979 | BU926668 | 486 | 57 | 149 | 38 | 7e−16 | regulation of transcription\, DNA- |
| | | | | | | | | | | | dependent |
| 8805 | 2.012034478 | down | 0.000696119 | 0.002793693 | BG650674 | 514 | 106 | 169 | 62 | 4e−52 | |
| 8806 | 2.011243279 | down | 0.0000387 | 0.00035347 | BI941830 | 387 | 8 | 26 | 30 | 0.50 | Flavanone 3′ hydroxylase (F3′H1) |
| | | | | | | | | | | | psuedogene, mRNA sequence, |
| | | | | | | | | | | | proteolysis and peptidolysis |
| 8807 | 2.010986276 | down | 0.0000154 | 0.000189375 | CD410293 | 595 | 63 | 88 | 71 | 3e−30 | transport |
| 8808 | 2.010826108 | down | 0.0000784 | 0.000578707 | BE347146 | 543 | 91 | 181 | 50 | 2e−45 | viral life cycle |
| 8809 | 2.010789644 | down | 0.0000287 | 0.000288411 | AW277435 | 385 | 13 | 41 | 31 | 2.4 | metabolism |
| 8810 | 2.010752457 | down | 0.000192829 | 0.00109712 | BI974314 | 560 | 113 | 140 | 80 | 3e−63 | cysteine biosynthesis from serine |
| 8811 | 2.010514205 | down | 0.000168204 | 0.000997269 | CK606408 | 739 | 152 | 174 | 87 | 2e−84 | protein biosynthesis |
| 8812 | 2.010420589 | down | 0.00016628 | 0.000988224 | BU089765 | 324 | 44 | 113 | 38 | 1e−13 | |
| 8813 | 2.009221975 | down | 0.00018271 | 0.001055319 | BG508965 | 417 | 19 | 28 | 67 | 1e−05 | multidrug transport |
| 8814 | 2.00920058 | down | 0.000276164 | 0.001427071 | BI893677 | 447 | 65 | 100 | 65 | 3e−34 | |
| 8815 | 2.008998618 | down | 0.0000743 | 0.000557886 | CD412322 | 488 | 94 | 129 | 72 | 3e−48 | male meiosis cytokinesis |
| 8816 | 2.00898247 | down | 0.0000321 | 0.000312465 | CA799417 | 687 | 53 | 97 | 54 | 2e−24 | |
| 8817 | 2.00853603 | down | 0.001050953 | 0.003778129 | BQ296811 | 574 | 26 | 43 | 60 | 3e−08 | |
| 8818 | 2.008197084 | down | 0.0000594 | 0.000477051 | CK605598 | 545 | 16 | 41 | 39 | 0.011 | |
| 8819 | 2.008099548 | down | 0.0000197 | 0.000222117 | AI440690 | 420 | 69 | 90 | 76 | 2e−36 | |
| 8820 | 2.008056002 | down | 0.001454269 | 0.004764376 | CF806620 | 649 | 166 | 212 | 78 | 3e−99 | |
| 8821 | 2.008008482 | down | 0.0000927 | 0000652791 | CF806124 | 712 | 111 | 192 | 57 | 2e−55 | electron transport |
| 8822 | 2.007961022 | down | 0.001777306 | 0.00550803 | AW100048 | 397 | 12 | 23 | 52 | 0.23 | carbohydrate metabolism |
| 8823 | 2.007876257 | down | 0.0000197 | 0.000221672 | BE347054 | 473 | 38 | 67 | 56 | 3e−13 | |
| 8824 | 2.007728799 | down | 0.0000161 | 0.000194621 | CD407646 | 653 | 124 | 147 | 84 | 4e−74 | asparagine biosynthesis |
| 8825 | 2.007692401 | down | 0.000248532 | 0.001322246 | BU760697 | 626 | 122 | 196 | 62 | 1e−68 | |
| 8826 | 2.007108762 | down | 0.000143368 | 0.000889286 | BQ610029 | 457 | 50 | 93 | 53 | 8e−22 | |
| 8827 | 2.006489663 | down | 0.0000513 | 0.000430266 | BE823887 | 613 | 63 | 92 | 68 | 7e−31 | |
| 8828 | 2.006293581 | down | 0.00066701 | 0.002712029 | BM086344 | 427 | 56 | 85 | 65 | 2e−30 | |
| 8829 | 2.006047026 | down | 0.0000491 | 0.000417918 | BU080831 | 441 | 50 | 81 | 61 | 3e−21 | |
| 8830 | 2.005955398 | down | 0.000103991 | 0.000707453 | BI968313 | 546 | 58 | 128 | 45 | 8e−30 | metabolism |
| 8831 | 2.005871803 | down | 0.000042 | 0.000375329 | BU547296 | 664 | 93 | 125 | 74 | 7e−51 | |
| 8832 | 2.005690331 | down | 0.000110624 | 0.000738815 | BE658257 | 788 | 100 | 117 | 85 | 4e−49 | transport |
| 8833 | 2.005554142 | down | 0.0000208 | 0.000230283 | BU084534 | 357 | 29 | 50 | 58 | 1e−10 | |
| 8834 | 2.00547534 | down | 0.000244705 | 0.001308203 | AW349133 | 664 | 63 | 126 | 50 | 6e−24 | |
| 8835 | 2.005380111 | down | 0.000131347 | 0.000837084 | CA852761 | 303 | 21 | 58 | 36 | 0.38 | transport |
| 8836 | 2.00528989 | down | 0.000123215 | 0.000800039 | BU083463 | 521 | 141 | 167 | 84 | 7e−81 | protein amino acid phosphorylation |
| 8837 | 2.005101029 | down | 0.000479691 | 0.002134269 | BI968510 | 770 | 115 | 152 | 75 | 8e−63 | |
| 8838 | 2.005050615 | down | 0.000128319 | 0.000823368 | BQ785725 | 599 | 45 | 90 | 50 | 6e−19 | |
| 8839 | 2.004949808 | down | 0.000509377 | 0.002227095 | BM177179 | 611 | 63 | 90 | 10 | 9e−29 | histidine biosynthesis |
| 8840 | 2.00458784 | down | 0.000238921 | 0.001283657 | BE820716 | 290 | 20 | 36 | 55 | 8e−07 | |
| 8841 | 2.004030016 | down | 0.000144038 | 0.000891828 | BU545833 | 602 | 71 | 88 | 80 | 1e−34 | UV protection |
| 8842 | 2.003932317 | down | 0.0000732 | 0.000552236 | BQ079820 | 420 | 102 | 131 | 17 | 8e−57 | |
| 8843 | 2.00346353 | down | 0.0000493 | 0.000418849 | BG653618 | 517 | 105 | 148 | 70 | 4e−57 | isoprenoid biosynthesis |
| 8844 | 2.003376083 | down | 0.000169761 | 0.001003657 | BG651980 | 522 | 78 | 91 | 85 | 5e−43 | mRNA splicing |
| 8845 | 2.003158437 | down | 0.000025 | 0.000263044 | AW350383 | 675 | 107 | 153 | 69 | 1e−56 | electron transport |
| 8846 | 2.002535558 | down | 0.000199838 | 0.001127618 | BQ785832 | 452 | 16 | 56 | 28 | 1.6 | |
| 8847 | 2.002524171 | down | 0.000119202 | 0.00078058 | AI966268 | 569 | 72 | 93 | 77 | 2e−35 | |
| 8848 | 2.002472461 | down | 0.000503063 | 0.00220667 | AI941451 | 549 | 119 | 180 | 66 | 9e−64 | mitochondrial translocation |
| 8849 | 2.002313747 | down | 0.000167521 | 0.000994196 | CF808533 | 733 | 231 | 233 | 99 | e−135 | selenocysteine incorporation |
| 8850 | 2.002211739 | down | 0.000140539 | 0.000876366 | BU544285 | 547 | 129 | 142 | 90 | 4e−68 | |
| 8851 | 2.002137054 | down | 0.0000116 | 0.000155212 | CF807318 | 632 | 110 | 139 | 79 | 5e−59 | D-ribose metabolism |
| 8852 | 2.002002247 | down | 0.0000666 | 0.000517279 | BG650417 | 359 | 21 | 52 | 40 | 0.68 | |
| 8853 | 2.001431687 | down | 0.000113744 | 0.000753366 | BM307959 | 494 | 46 | 90 | 51 | 6e−23 | |
| 8854 | 2.001365188 | down | 0.000116547 | 0.000767423 | AI437705 | 471 | 82 | 145 | 56 | 8e−44 | |
| 8855 | 2.00123112 | down | 0.0000302 | 0.00029957 | AW432768 | 225 | 13 | 38 | 34 | 0.65 | negative regulation of flower |
| | | | | | | | | | | | development |
| 8856 | 2.001059861 | down | 0.0000499 | 0.000422169 | BE660090 | 540 | 73 | 127 | 57 | 4e−29 | |
| 8857 | 2.000726426 | down | 0.0000465 | 0.000403528 | BE659702 | 477 | 34 | 47 | 72 | 2e−16 | |
| 8858 | 2.000534521 | down | 0.0000179 | 0.00020806 | BQ298220 | 420 | 25 | 33 | 75 | 2e−10 | |
| 8859 | 2.000293088 | down | 0.001050774 | 0.003777844 | BQ296797 | 415 | 39 | 149 | 26 | 2e−05 | response to light |
| 8860 | 2.00020914 | down | 0.000135607 | 0.000854676 | BU084724 | 488 | 13 | 24 | 54 | 0.85 | |
| 8861 | 102.4086652 | up | 2.17E−09 | 0.00000146 | BI321308 | 366 | 12 | 31 | 38 | 1.6 | |
| 8862 | 86.51359106 | up | 9.63E−08 | 0.00000844 | M94012.1 | | | | | | dehydrin-like protein |
| 8863 | 66.42860337 | up | 4.52E−09 | 0.00000202 | AF117884.1 | | | | | | seed maturation protein PM30 |
| 8864 | 64.25697861 | up | 1.62E−10 | 0.000000589 | BF324269 | 563 | 36 | 61 | 59 | 1e−12 | |
| 8865 | 51.63159713 | up | 5.28E−09 | 0.00000216 | AW569917 | 347 | 20 | 53 | 37 | 0.49 | |
| 8866 | 49.01876256 | up | 8.96E−11 | 0.000000589 | BU548370 | 583 | 84 | 119 | 70 | 2e−45 | carbohydrate biosynthesis |
| 8867 | 46.02277472 | up | 1.61E−08 | 0.00000353 | AF117884.1 | | | | | | seed maturation protein PM30 |
| 8868 | 44.17987983 | up | 2E−10 | 0.000000589 | BI893889 | 535 | 13 | 39 | 33 | 1.3 | |
| 8869 | 38.2422535 | up | 1.13E−10 | 0.000000589 | BU550698 | 642 | 25 | 92 | 27 | 2e−07 | |
| 8870 | 35.90880635 | up | 0.000000121 | 0.00000972 | BM886682 | 461 | 80 | 147 | 54 | 3e−41 | |
| 8871 | 33.73985349 | up | 5.69E−11 | 0.000000589 | BE823727 | 653 | 52 | 111 | 46 | 9e−24 | |
| 8872 | 32.2980714 | up | 0.00000175 | 0.0000466 | AF004806.1 | | | | | | 24 kDa seed maturation protein |
| 8873 | 31.36943355 | up | 3.71E−11 | 0.000000589 | BI974508 | 527 | 48 | 121 | 39 | 8e−19 | |
| 8874 | 30.97196324 | up | 0.00000201 | 0.0000512 | AW569963 | 393 | 25 | 43 | 58 | 2e−08 | |
| 8875 | 29.07132921 | up | 8.64E−10 | 0.000000915 | AW597300 | 380 | 21 | 27 | 77 | 8e−06 | |
| 8876 | 27.43851138 | up | 6.23E−10 | 0.00000078 | CD393365 | 581 | 26 | 88 | 29 | 2e−08 | |
| 8877 | 26.31917741 | up | 1.79E−08 | 0.00000368 | BM528173 | 548 | 122 | 158 | 77 | 9e−66 | |
| 8878 | 23.95732798 | up | 8.23E−08 | 0.00000782 | Z22872.1 | | | | | | Shi-shi 51 kDa seed maturation protein |
| | | | | | | | | | | | (pGmPM10) /// Lea protein |
| 8879 | 23.84906851 | up | 1.26E−09 | 0.0000011 | AF109720.1 | | | | | | seed maturation protein PM35 |
| 8880 | 23.66599522 | up | 0.00000124 | 0.0000373 | AW568567 | 438 | 50 | 63 | 79 | 3e−24 | |
| 8881 | 22.92493266 | up | 1.87E−08 | 0.00000371 | M93568.1 | | | | | | maturation-associated protein |
| 8882 | 22.81308051 | up | 2.35E−10 | 0.000000589 | AF169025.1 | | | | | | seed maturation protein PM41 |
| 8883 | 22.5390291 | up | 4.51E−10 | 0.000000745 | BU548646 | 493 | 26 | 112 | 23 | 1.5 | translational termination |
| 8884 | 21.91377239 | up | 3.5E−10 | 0.000000658 | X63565.1 | | | | | | soybean seed maturation polypeptides |
| 8885 | 21.53781686 | up | 1.58E−08 | 0.00000353 | BE474472 | 420 | 21 | 72 | 29 | 0.35 | |
| 8886 | 21.25216009 | up | 0.00000132 | 0.0000386 | CF809067 | 638 | 117 | 161 | 72 | 2e−61 | response to oxidative stress |
| 8887 | 21.09343405 | up | 2.47E−08 | 0.00000414 | BU090518 | 412 | 16 | 44 | 36 | 0.99 | |
| 8888 | 19.24404851 | up | 3.37E−08 | 0.00000492 | BI316837 | 467 | 42 | 47 | 89 | 2e−20 | proline biosynthesis |
| 8889 | 18.72292884 | up | 1.31E−09 | 0.0000011 | BM095037 | 514 | 41 | 94 | 43 | 4e−18 | |
| 8890 | 18.0808727 | up | 2.96E−10 | 0.000000618 | BG363535 | 486 | 79 | 142 | 55 | 7e−37 | |
| 8891 | 12.849467 | up | 8.07E−08 | 0.00000772 | BM887020 | 541 | 125 | 183 | 68 | 3e−78 | |
| 8892 | 12.54809475 | up | 0.001039903 | 0.00375095 | BE823689 | 644 | 98 | 158 | 62 | 9e−51 | |
| 8893 | 12.32265564 | up | 0.00013758 | 0.000863927 | U21724.1 | | | | | | Hsp22.5 |
| 8894 | 12.19573418 | up | 0.0000438 | 0.00038769 | CF805621 | 542 | 105 | 153 | 68 | 3e−55 | |
| 8895 | 12.15807085 | up | 0.001687806 | 0.005316685 | AF202731.1 | | | | | | chitinase class I |
| 8896 | 12.13784114 | up | 4.67E−08 | 0.00000572 | BU760782 | 538 | 68 | 124 | 54 | 3e−26 | |
| 8897 | 17.87184756 | up | 0.000000258 | 0.0000148 | AW569502 | 446 | 71 | 119 | 69 | 1e−36 | |
| 8898 | 17.33372405 | up | 0.0000139 | 0.00017604 | AW620208 | 417 | 32 | 52 | 61 | 8e−11 | |
| 8899 | 16.24786989 | up | 0.000000171 | 0.0000117 | Z22872.1 | | | | | | Lea protein |
| 8900 | 15.36317377 | up | 0.0000285 | 0.000287328 | CF807875 | 644 | 107 | 155 | 69 | 3e−57 | |
| 8901 | 15.27094907 | up | 0.00000212 | 0.000053 | BU550058 | 631 | 84 | 132 | 63 | 5e−43 | |
| 8902 | 14.83768636 | up | 0.000000269 | 0.000015 | AW306163 | 394 | 48 | 58 | 82 | 5e−23 | |
| 8903 | 14.47550482 | up | 0.000000172 | 0.0000118 | CA782738 | 435 | 22 | 43 | 51 | 6e−07 | carbohydrate metabolism |
| 8904 | 14.40425229 | up | 0.0000091 | 0.000131164 | BM143164 | 476 | 51 | 113 | 45 | 2e−20 | |
| 8905 | 14.14343323 | up | 2.1E−09 | 0.00000146 | CF806037 | 678 | 97 | 134 | 72 | 3e−55 | cold acclimation |
| 8906 | 13.96241702 | up | 0.000000264 | 0.0000149 | CK606334 | 717 | 118 | 208 | 56 | 1e−65 | Glutathione S-transferase GST 8, |
| | | | | | | | | | | | transport |
| 8907 | 13.78345945 | up | 0.000636751 | 0.002624742 | CD392150 | 665 | 64 | 191 | 33 | 1e−14 | |
| 8908 | 13.77192719 | up | 0.000000547 | 0.0000236 | BF325207 | 483 | 29 | 71 | 40 | 3e−13 | |
| 8909 | 9.359665018 | up | 6.24E−08 | 0.00000682 | BE823818 | 709 | 103 | 190 | 54 | 1e−56 | electron transport |
| 8910 | 9.346469654 | up | 3.42E−09 | 0.00000183 | BQ740664 | 298 | 16 | 53 | 30 | 4.2 | |
| 8911 | 9.270204026 | up | 0.002333401 | 0.006673603 | AW569992 | 391 | 88 | 129 | 68 | 8e−47 | |
| 8912 | 9.231589928 | up | 0.000000506 | 0.0000224 | CF806325 | 702 | 169 | 228 | 74 | e−102 | |
| 8913 | 9.174756652 | up | 6.33E−08 | 0.00000688 | AW396773 | 651 | 52 | 119 | 43 | 1e−21 | |
| 8914 | 9.174720488 | up | 0.0000012 | 0.0000367 | BU090523 | 379 | 22 | 92 | 23 | 0.12 | electron transport |
| 8915 | 9.106121593 | up | 0.000319865 | 0.001589219 | AW567704 | 355 | 19 | 51 | 37 | 0.51 | |
| 8916 | 8.995995013 | up | 0.000000313 | 0.0000167 | BE821604 | 652 | 49 | 94 | 52 | 3e−23 | |
| 8917 | 8.973788629 | up | 0.00000142 | 0.0000406 | AW132190 | | | | | | |
| 8918 | 8.78820338 | up | 0.000049 | 0.00041694 | BM568072 | 579 | 164 | 193 | 84 | 9e−94 | |
| 8919 | 8.743068589 | up | 0.00000102 | 0.0000339 | AF169023.1 | | | | | | seed maturation protein PM38 |
| 8920 | 4.232735741 | up | 0.005469887 | 0.012193701 | BE823692 | 506 | 24 | 49 | 48 | 3e−11 | |
| 8921 | 4.22883439 | up | 0.0001197 | 0.000782756 | AF529303.1 | | | | | | |
| 8922 | 4.227055936 | up | 0.001062677 | 0.003809463 | CF809367 | 351 | 29 | 42 | 69 | 2e−12 | proteolysis and peptidolysis |
| 8923 | 13.54897678 | up | 0.000000327 | 0.0000172 | CK606334 | 717 | 118 | 208 | 56 | 1e−65 | Glutathione S-transferase GST 8, |
| | | | | | | | | | | | transport |
| 8924 | 13.54621618 | up | 2.81E−08 | 0.00000439 | CD403280 | 412 | 13 | 36 | 36 | 6.4 | xyloglucan biosynthesis |
| 8925 | 13.47665749 | up | 0.000000582 | 0.0000243 | BE658702 | 338 | 16 | 65 | 24 | 2.5 | lipid metabolism |
| 8926 | 13.4026527 | up | 4.63E−10 | 0.000000745 | CD403249 | 558 | 26 | 39 | 66 | 3e−10 | lipid metabolism |
| 8927 | 11.72194491 | up | 0.00000013 | 0.0000101 | AI938364 | 385 | 13 | 40 | 32 | 1.4 | metabolism |
| 8928 | 11.50903173 | up | 1.62E−08 | 0.00000353 | CF807476 | 668 | 81 | 114 | 71 | 3e−40 | |
| 8929 | 11.41790295 | up | 1.01E−08 | 0.00000293 | M80666.1 | | | | | | maturation polypeptide |
| 8930 | 11.41301274 | up | 0.0000167 | 0.0001987 | BE583765 | 559 | 68 | 92 | 73 | 5e−31 | response to heat |
| 8931 | 11.3760445 | up | 0.000000363 | 0.0000182 | AF243364.1 | | | | | | glutathione S-transferase GST 9 |
| 8932 | 11.30323045 | up | 0.0000333 | 0.000320524 | BU546301 | 655 | 65 | 145 | 44 | 3e−31 | |
| 8933 | 7.966585828 | up | 1.36E−08 | 0.00000333 | AW757140 | 416 | 15 | 66 | 22 | 3.9 | |
| 8934 | 7.909985456 | up | 0.0000799 | 0.000587493 | AW351262 | 590 | 60 | 216 | 27 | 9e−09 | |
| 8935 | 7.907425473 | up | 0.0000267 | 0.000274765 | BE659724 | 458 | 53 | 76 | 69 | 4e−27 | |
| 8936 | 6.052528683 | up | 0.00000232 | 0.000056 | BU544413 | 491 | 35 | 75 | 46 | 2e−12 | |
| 8937 | 11.1207226 | up | 0.00000254 | 0.0000595 | BE824410 | 535 | 68 | 104 | 65 | 2e−32 | |
| 8938 | 11.11118349 | up | 0.000177318 | 0.001034053 | CF807875 | 644 | 107 | 155 | 69 | 3e−57 | |
| 8939 | 11.04943641 | up | 0.000233942 | 0.00126524 | BE807374 | 332 | 50 | 112 | 44 | 3e−21 | |
| 8940 | 10.95100791 | up | 0.000000776 | 0.0000291 | U02966.1 | | | | | | Shi-shi 51 kDa seed maturation protein |
| | | | | | | | | | | | (pGmPM10) |
| 8941 | 10.8610407 | up | 8.76E−10 | 0.000000915 | BI968048 | 710 | 80 | 110 | 72 | 1e−39 | |
| 8942 | 10.75435614 | up | 0.00000153 | 0.0000426 | AW164229 | 209 | 12 | 30 | 40 | 1.1 | proteolysis and peptidolysis |
| 8943 | 10.70527725 | up | 3.76E−09 | 0.00000189 | CD408988 | 562 | 22 | 46 | 47 | 3e−11 | regulation of transcription\, DNA- |
| | | | | | | | | | | | dependent |
| 8944 | 10.62757481 | up | 1.37E−08 | 0.00000334 | BU549133 | 563 | 78 | 151 | 51 | 8e−34 | |
| 8945 | 10.62180878 | up | 0.000962652 | 0.003554938 | BG157124 | | | | | | |
| 8946 | 10.59864946 | up | 0.00000161 | 0.000044 | AI966352 | 286 | 12 | 24 | 50 | 3.2 | Malate synthase (MS) mRNA, 3′ end. |
| 8947 | 10.28104734 | up | 1.03E−08 | 0.00000293 | CF806120 | 421 | 15 | 27 | 55 | 0.21 | Hydroxyproline-rich glycoprotein (hrgp), |
| | | | | | | | | | | | apoptosis |
| 8948 | 10.20525969 | up | 0.0000254 | 0.000265791 | AW569888 | 283 | 9 | 25 | 36 | 9.2 | |
| 8949 | 10.06274891 | up | 0.001036392 | 0.003741875 | BI942525 | 243 | 16 | 52 | 30 | 0.83 | |
| 8950 | 10.05860336 | up | 0.000988639 | 0.003622667 | CF806263 | 637 | 95 | 154 | 61 | 3e−52 | |
| 8951 | 9.903732699 | up | 1.27E−09 | 0.0000011 | AW396311 | 347 | 44 | 85 | 51 | 3e−18 | transport |
| 8952 | 9.86661586 | up | 0.000122426 | 0.000795876 | U21723.1 | | | | | | Hsp22.3 |
| 8953 | 9.806544898 | up | 7.52E−08 | 0.00000744 | BE823818 | 709 | 103 | 190 | 54 | 1e−56 | electron transport |
| 8954 | 9.699483867 | up | 0.002450868 | 0.006904486 | U59626.1 | | | | | | LEA protein |
| 8955 | 9.646552164 | up | 0.00000322 | 0.0000687 | CD408999 | 514 | 24 | 95 | 25 | 2.1 | proton transport |
| 8956 | 9.474409481 | up | 0.0000336 | 0.000323082 | BF595112 | 297 | 17 | 53 | 32 | 0.38 | |
| 8957 | 9.42405865 | up | 8.28E−08 | 0.00000782 | BI945193 | | | | | | |
| 8958 | 9.403548842 | up | 0.000000006 | 0.00000221 | CD393365 | 581 | 26 | 88 | 29 | 2e−08 | |
| 8959 | 9.387010859 | up | 3.62E−08 | 0.00000504 | U66316.1 | | | | | | desiccation protective protein LEA5 |
| 8960 | 9.386068522 | up | 0.00000128 | 0.0000382 | BM886862 | 437 | 21 | 65 | 32 | 0.39 | |
| 8961 | 8.668659244 | up | 0.0000116 | 0.000155201 | L35272.1 | | | | | | Heat shock protein (SB100) |
| 8962 | 8.592741905 | up | 6.21E−08 | 0.00000682 | BM886596 | 572 | 12 | 35 | 34 | 2.6 | |
| 8963 | 8.531295763 | up | 2.85E−09 | 0.00000167 | CD406679 | 627 | 11 | 21 | 52 | 0.46 | regulation of transcription\, DNA- |
| | | | | | | | | | | | dependent |
| 8964 | 8.528406083 | up | 0.003555663 | 0.008968197 | AW569992 | 391 | 88 | 129 | 68 | 8e−47 | |
| 8965 | 8.525587766 | up | 0.000000161 | 0.0000114 | CA802334 | 455 | 7 | 24 | 29 | 6.2 | |
| 8966 | 8.414260859 | up | 0.000000035 | 0.00000495 | BE658779 | 389 | 20 | 51 | 39 | 0.012 | |
| 8967 | 8.341658831 | up | 1.38E−08 | 0.00000334 | CA851984 | 229 | 12 | 38 | 31 | 9.3 | |
| 8968 | 8.271929228 | up | 0.00000162 | 0.0000441 | AW567661 | 365 | 15 | 49 | 30 | 1.6 | leaf morphogenesis |
| 8969 | 8.256432013 | up | 8.37E−08 | 0.00000783 | BI974374 | 535 | 107 | 155 | 69 | 6e−56 | response to oxidative stress |
| 8970 | 8.235477069 | up | 0.0000441 | 0.000389174 | BM094572 | 536 | 62 | 143 | 43 | 4e−26 | |
| 8971 | 8.106179544 | up | 5.89E−08 | 0.00000668 | BI892562 | 467 | 34 | 45 | 75 | 7e−16 | amino acid transport |
| 8972 | 8.029552432 | up | 0.00000978 | 0.000138062 | CF806487 | 376 | 46 | 92 | 50 | 9e−21 | |
| 8973 | 8.020562162 | up | 1.57E−08 | 0.00000353 | CF807204 | 164 | 16 | 36 | 44 | 0.23 | |
| 8974 | 7.90029427 | up | 0.000117567 | 0.000772525 | BG651692 | 458 | 43 | 62 | 69 | 3e−23 | G-protein coupled receptor protein |
| | | | | | | | | | | | signaling pathway |
| 8975 | 7.885465005 | up | 2.43E−09 | 0.0000015 | BM522852 | 427 | 45 | 59 | 76 | 8e−22 | polysaccharide catabolism |
| 8976 | 7.766534353 | up | 0.0000176 | 0.000206049 | BE821978 | 482 | 73 | 105 | 69 | 3e−39 | oligopeptide transport |
| 8977 | 7.76336193 | up | 1.03E−08 | 0.00000293 | AW164547 | 438 | 27 | 35 | 77 | 8e−12 | response to cold |
| 8978 | 7.716899883 | up | 1.87E−08 | 0.00000371 | BU544815 | 582 | 66 | 114 | 57 | 3e−29 | |
| 8979 | 7.709670937 | up | 0.00000841 | 0.000124293 | BM270844 | 477 | 13 | 25 | 52 | 0.042 | protein modification |
| 8980 | 7.67644332 | up | 0.0000518 | 0.000433459 | D63781.1 | | | | | | epoxide hydrolase |
| 8981 | 7.66603982 | up | 7.21E−09 | 0.00000235 | U41475.1 | | | | | | phosphoinositide-specific phospholipase |
| | | | | | | | | | | | C P25 |
| 8982 | 7.665764972 | up | 0.000000744 | 0.0000284 | CD394008 | 558 | 20 | 66 | 30 | 1.4 | |
| 8983 | 7.616362454 | up | 0.001176798 | 0.004091705 | BG649950 | | | | | | |
| 8984 | 7.456086341 | up | 0.00000613 | 0.000102873 | AF243369.1 | | | | | | glutathione S-transferase GST 14 |
| 8985 | 7.420615617 | up | 0.000011 | 0.000149798 | CF806325 | 702 | 169 | 228 | 74 | e−102 | |
| 8986 | 7.343389621 | up | 2.68E−08 | 0.00000432 | CF807696 | 567 | 98 | 157 | 62 | 2e−50 | aromatic compound metabolism |
| 8987 | 7.337577543 | up | 0.000000527 | 0.000023 | BE657436 | 777 | 95 | 147 | 64 | 1e−48 | |
| 8988 | 7.2884169 | up | 0.001269245 | 0.004321975 | BU549129 | 475 | 39 | 106 | 36 | 1e−12 | apoptosis |
| 8989 | 7.224762942 | up | 2.67E−08 | 0.00000432 | BM093715 | 441 | 19 | 26 | 73 | 9e−06 | |
| 8990 | 7.21960327 | up | 0.0000294 | 0.000293401 | BE608018 | 571 | 113 | 186 | 60 | 1e−66 | |
| 8991 | 7.211182143 | up | 0.007578041 | 0.015428441 | AW569992 | 391 | 88 | 129 | 68 | 8e−47 | |
| 8992 | 7.200737305 | up | 0.0000013 | 0.0000384 | AF004807.1 | | | | | | dehydrin |
| 8993 | 7.118246317 | up | 0.0000106 | 0.000146441 | BI469834 | 406 | 14 | 36 | 38 | 0.065 | |
| 8994 | 7.117050543 | up | 0.000643915 | 0.002644706 | AI496603 | 391 | 39 | 124 | 31 | 4e−11 | |
| 8995 | 7.113490563 | up | 0.000011 | 0.00014993 | CA935537 | 377 | 17 | 28 | 60 | 0.001 | |
| 8996 | 7.052256265 | up | 0.000000694 | 0.0000273 | BI974591 | 521 | 123 | 172 | 71 | 1e−69 | response to oxidative stress |
| 8997 | 6.987540012 | up | 0.0000222 | 0.000241835 | BF325331 | 303 | 20 | 39 | 51 | 1e−05 | response to osmotic stress |
| 8998 | 6.977996134 | up | 5.64E−09 | 0.00000219 | CA937233 | 414 | 11 | 32 | 34 | 2.9 | |
| 8999 | 6.927031643 | up | 0.0000014 | 0.0000404 | CD395789 | 592 | 21 | 75 | 28 | 3.5 | |
| 9000 | 6.747657931 | up | 0.0000601 | 0.000480712 | BU544169 | 529 | 107 | 139 | 76 | 9e−61 | protein folding |
| 9001 | 6.63222819 | up | 0.00000102 | 0.0000339 | AW349200 | | | | | | |
| 9002 | 6.591005891 | up | 2.52E−08 | 0.00000418 | BI945611 | 549 | 50 | 64 | 78 | 3e−23 | response to oxidative stress |
| 9003 | 6.572735778 | up | 0.000532035 | 0.002303403 | AW351136 | 691 | 80 | 99 | 80 | 4e−43 | electron transport |
| 9004 | 6.488051385 | up | 0.00000916 | 0.000131953 | BE657172 | 546 | 19 | 67 | 28 | 1.4 | |
| 9005 | 6.465780319 | up | 0.000000641 | 0.0000258 | AW308946 | 222 | 11 | 40 | 27 | 1.9 | |
| 9006 | 6.418033473 | up | 0.007304116 | 0.015041689 | AW567705 | 382 | 15 | 41 | 36 | 0.62 | |
| 9007 | 6.308176505 | up | 0.000000134 | 0.0000103 | AW396773 | 651 | 52 | 119 | 43 | 1e−21 | |
| 9008 | 6.295833409 | up | 0.0000815 | 0.000595843 | BM887146 | 546 | 127 | 182 | 69 | 6e−70 | response to wounding |
| 9009 | 6.256651006 | up | 0.000163588 | 0.000975922 | CF805621 | 542 | 105 | 153 | 68 | 3e−55 | |
| 9010 | 6.242987506 | up | 0.000314849 | 0.00157378 | CF806810 | 491 | 20 | 54 | 37 | 0.001 | sodium ion transport |
| 9011 | 6.23921087 | up | 0.0000171 | 0.000202007 | BU551266 | 366 | 29 | 80 | 36 | 7e−07 | |
| 9012 | 6.035317022 | up | 2.76E−08 | 0.00000439 | BU547059 | 656 | 21 | 54 | 38 | 0.002 | |
| 9013 | 6.031672831 | up | 0.000646704 | 0.002653263 | AI437934 | 413 | 38 | 74 | 51 | 1e−15 | polygalacturonase inhibitor protein |
| 9014 | 6.012270062 | up | 0.004999249 | 0.011430414 | U66317.1 | | | | | | Em protein |
| 9015 | 5.955248124 | up | 0.0000692 | 0.000531387 | CD406632 | 628 | 38 | 96 | 39 | 1e−18 | |
| 9016 | 5.93861517 | up | 0.00000693 | 0.000110527 | AW350679 | 752 | 122 | 194 | 62 | 5e−70 | |
| 9017 | 5.936510068 | up | 1.17E−08 | 0.00000304 | BE823095 | 543 | 28 | 56 | 50 | 2e−07 | protein amino acid dephosphorylation |
| 9018 | 5.932864724 | up | 6.81E−09 | 0.00000229 | BE823095 | 543 | 28 | 56 | 50 | 2e−07 | protein amino acid dephosphorylation |
| 9019 | 5.928439594 | up | 0.0000576 | 0.00046747 | CA802310 | 430 | 38 | 76 | 50 | 8e−20 | |
| 9020 | 5.902542539 | up | 0.0000427 | 0.000380213 | BG406552 | 522 | 32 | 58 | 55 | 8e−11 | sulfate transport |
| 9021 | 5.899183602 | up | 0.000000435 | 0.0000205 | CF808945 | 605 | 17 | 56 | 30 | 6.3 | |
| 9022 | 5.863061239 | up | 6.12E−09 | 0.00000221 | BI699825 | 492 | 51 | 72 | 70 | 1e−27 | |
| 9023 | 5.132914009 | up | 0.00027695 | 0.00143015 | CF808111 | 540 | 112 | 139 | 80 | 7e−58 | response to heat |
| 9024 | 5.074369245 | up | 6.07E−08 | 0.00000678 | AF169021.1 | | | | | | seed maturation protein PM36 |
| 9025 | 5.066683447 | up | 0.00000968 | 0.000137088 | AW201851 | | | | | | |
| 9026 | 5.825065863 | up | 0.00000395 | 0.0000781 | BU551360 | 542 | 79 | 103 | 76 | 3e−41 | electron transport |
| 9027 | 5.819642297 | up | 3.04E−08 | 0.0000046 | BM271479 | 248 | 10 | 23 | 43 | 3.1 | |
| 9028 | 5.767089994 | up | 0.0000106 | 0.000146441 | BF324988 | 511 | 65 | 108 | 60 | 6e−37 | cell wall organization and biogenesis |
| | | | | | | | | | | | (sensu Magnoliophyta) |
| 9029 | 5.760762448 | up | 0.000000227 | 0.0000139 | CF807696 | 567 | 98 | 157 | 62 | 2e−50 | aromatic compound metabolism |
| 9030 | 5.746824206 | up | 0.0000225 | 0.000243882 | BE659011 | 290 | 10 | 25 | 40 | 5.4 | |
| 9031 | 5.744305429 | up | 0.001752473 | 0.005455554 | CF806693 | 662 | 108 | 163 | 66 | 3e−55 | transition metal ion homeostasis |
| 9032 | 5.743455929 | up | 0.000668969 | 0.002717342 | CF807731 | 538 | 93 | 130 | 71 | 9e−55 | proteolysis and peptidolysis |
| 9033 | 5.729504122 | up | 0.004096173 | 0.009933174 | AW201364 | 419 | 51 | 65 | 78 | 5e−25 | transition metal ion homeostasis |
| 9034 | 5.72041738 | up | 0.0000893 | 0.000636093 | BI701238 | 534 | 85 | 172 | 49 | 2e−51 | |
| 9035 | 5.718029723 | up | 0.0000521 | 0.000435108 | AW279540 | 549 | 107 | 189 | 56 | 5e−54 | protein amino acid dephosphorylation |
| 9036 | 5.677772836 | up | 0.0000014 | 0.0000404 | CF806991 | 438 | 20 | 38 | 52 | 6e−05 | |
| 9037 | 5.666703177 | up | 0.005227025 | 0.011798199 | BU549021 | 641 | 90 | 163 | 55 | 7e−40 | |
| 9038 | 5.628715634 | up | 0.000403116 | 0.001879607 | BF423860 | 313 | 13 | 31 | 41 | 1.9 | proteolysis and peptidolysis |
| 9039 | 5.615943908 | up | 0.0000426 | 0.000379932 | AY126715.1 | | | | | | galactinol synthase |
| 9040 | 5.605331836 | up | 0.000000628 | 0.0000254 | CF808134 | 508 | 14 | 23 | 60 | 0.41 | sucrose transporter |
| 9041 | 5.604102364 | up | 0.0000459 | 0.000399603 | BI974382 | 488 | 66 | 102 | 64 | 6e−37 | glutathione S-transferase GST 14, |
| | | | | | | | | | | | aromatic amino acid family metabolism |
| 9042 | 5.572714482 | up | 0.00000124 | 0.0000373 | BI892702 | 408 | 23 | 58 | 39 | 0.023 | metabolism |
| 9043 | 5.545210746 | up | 0.00000575 | 0.0000987 | BE659535 | 782 | 15 | 46 | 32 | 7.3 | chromatin assembly/disassembly |
| 9044 | 5.529962662 | up | 0.000361239 | 0.00173334 | BI788213 | 528 | 15 | 35 | 42 | 0.014 | |
| 9045 | 5.522854155 | up | 0.000149415 | 0.000913984 | BM525537 | 504 | 14 | 27 | 51 | 0.41 | |
| 9046 | 5.521091696 | up | 0.000000035 | 0.00000495 | BE823010 | 656 | 55 | 140 | 39 | 2e−18 | dicarboxylic acid transport |
| 9047 | 5.512709726 | up | 0.0000813 | 0.000594564 | CD413437 | 544 | 20 | 46 | 43 | 1.4 | |
| 9048 | 5.505985751 | up | 0.00000397 | 0.0000783 | CF805740 | 544 | 38 | 61 | 62 | 7e−18 | |
| 9049 | 5.468526842 | up | 0.000345816 | 0.001677955 | AW569879 | 511 | 63 | 87 | 72 | 1e−32 | response to wounding |
| 9050 | 5.464261591 | up | 0.000602729 | 0.002519284 | BM188482 | 373 | 50 | 70 | 71 | 9e−26 | |
| 9051 | 5.444261043 | up | 0.000000243 | 0.0000144 | AW397631 | 401 | 59 | 75 | 78 | 9e−31 | GMFP7, metal ion transport |
| 9052 | 5.438832166 | up | 4.55E−08 | 0.00000565 | BM270250 | 246 | 17 | 49 | 34 | 1.1 | metabolism |
| 9053 | 5.426017764 | up | 0.000000134 | 0.0000103 | BE209406 | | | | | | |
| 9054 | 5.383127059 | up | 0.000502849 | 0.002205989 | BU925900 | 552 | 114 | 184 | 61 | 1e−61 | |
| 9055 | 5.379789152 | up | 0.000188366 | 0.001078747 | BQ473598 | 447 | 27 | 58 | 46 | 3e−06 | |
| 9056 | 5.340300008 | up | 0.0000436 | 0.000386227 | BU547701 | 627 | 127 | 151 | 84 | 6e−76 | electron transport |
| 9057 | 5.335299718 | up | 0.000000596 | 0.0000247 | BE822640 | 640 | 83 | 121 | 68 | 2e−45 | |
| 9058 | 5.310828266 | up | 0.00000119 | 0.0000365 | BI892702 | 408 | 23 | 58 | 39 | 0.023 | metabolism |
| 9059 | 5.30372019 | up | 0.000000451 | 0.000021 | AW156713 | 303 | 11 | 34 | 32 | 5.5 | protein amino acid phosphorylation |
| 9060 | 5.289764081 | up | 0.00000109 | 0.000035 | BU551096 | 405 | 13 | 52 | 25 | 0.42 | DNA repair |
| 9061 | 5.286148475 | up | 0.000000021 | 0.00000385 | BE021503 | 462 | 17 | 39 | 43 | 0.26 | |
| 9062 | 5.26617737 | up | 0.002098307 | 0.00619511 | BE659482 | 759 | 98 | 199 | 49 | 1e−43 | DNA metabolism |
| 9063 | 5.25837662 | up | 0.00000172 | 0.0000461 | CD404556 | 199 | 12 | 32 | 37 | 0.22 | cation transport |
| 9064 | 5.012753837 | up | 0.000567668 | 0.002420017 | AF117724.1 | | | | | | seed maturation protein PM28 |
| 9065 | 5.011624122 | up | 0.00144522 | 0.004747644 | AW705747 | 450 | 48 | 81 | 59 | 4e−22 | epoxide hydrolase, proteolysis and |
| | | | | | | | | | | | peptidolysis |
| 9066 | 4.997749814 | up | 0.00000112 | 0.0000355 | BG509281 | 463 | 52 | 116 | 44 | 4e−19 | two-component signal transduction |
| | | | | | | | | | | | system (phosphorelay) |
| 9067 | 4.984822952 | up | 0.0000546 | 0.000449783 | BE661789 | 648 | 56 | 73 | 76 | 1e−27 | potassium ion transport |
| 9068 | 4.973789346 | up | 0.000013 | 0.000168352 | CA802310 | 430 | 38 | 76 | 50 | 8e−20 | |
| 9069 | 4.960784064 | up | 0.000000083 | 0.00000783 | CF805794 | 549 | 134 | 176 | 76 | 2e−76 | |
| 9070 | 4.956026924 | up | 0.00000788 | 0.000119427 | CF805794 | 549 | 134 | 176 | 76 | 2e−76 | |
| 9071 | 4.944851381 | up | 0.0000079 | 0.000119582 | AF004810.1 | | | | | | late-embryogenesis abundant protein |
| 9072 | 4.915654019 | up | 0.00002 | 0.00022417 | BF425929 | 433 | 38 | 92 | 41 | 2e−08 | |
| 9073 | 4.902550059 | up | 0.00000179 | 0.0000473 | BU084758 | 480 | 35 | 40 | 87 | 7e−16 | negative regulation of flower |
| | | | | | | | | | | | development |
| 9074 | 4.89241938 | up | 0.000743239 | 0.002928578 | CA852440 | 342 | 31 | 42 | 73 | 3e−13 | proteolysis and peptidolysis |
| 9075 | 4.887817777 | up | 0.000000149 | 0.0000109 | CF921241 | 729 | 15 | 27 | 55 | 0.59 | Hydroxyproline-rich glycoprotein (hrgp), |
| | | | | | | | | | | | apoptosis |
| 9076 | 4.863804329 | up | 0.000146079 | 0.000900025 | BK000118.1 | | | | | | |
| 9077 | 4.862702374 | up | 0.0000473 | 0.000407798 | CF807451 | 190 | 37 | 60 | 61 | 4e−20 | |
| 9078 | 4.856092391 | up | 0.00000147 | 0.0000415 | CD403280 | 412 | 13 | 36 | 36 | 6.4 | xyloglucan biosynthesis |
| 9079 | 4.855781436 | up | 0.006194535 | 0.013338746 | BE822846 | 722 | 101 | 138 | 73 | 9e−58 | alcohol dehydrogenase 1, electron |
| | | | | | | | | | | | transport |
| 9080 | 4.841327884 | up | 0.00000212 | 0.000053 | AW310386 | 573 | 46 | 88 | 52 | 5e−18 | response to wounding |
| 9081 | 4.797032066 | up | 6.79E−08 | 0.00000722 | BE191618 | 417 | 31 | 60 | 51 | 2e−13 | |
| 9082 | 4.783414971 | up | 5.92E−08 | 0.00000668 | BF596503 | 374 | 96 | 124 | 77 | 4e−50 | protein amino acid dephosphorylation |
| 9083 | 4.782941374 | up | 0.00000356 | 0.0000731 | CD395871 | 608 | 13 | 40 | 32 | 0.98 | microsporogenesis |
| 9084 | 4.774025606 | up | 0.000200571 | 0.001130442 | BI701238 | 534 | 85 | 172 | 49 | 2e−51 | |
| 9085 | 4.769808301 | up | 0.0000659 | 0.000512768 | AW279540 | 549 | 107 | 189 | 56 | 5e−54 | protein amino acid dephosphorylation |
| 9086 | 4.750336786 | up | 0.000123995 | 0.000804543 | BM887385 | 246 | 11 | 24 | 45 | 1.9 | regulation of transcription\, DNA- |
| | | | | | | | | | | | dependent |
| 9087 | 4.724917647 | up | 0.00000929 | 0.000132842 | AW509471 | 497 | 58 | 169 | 34 | 3e−16 | seed maturation protein PM39, |
| | | | | | | | | | | | response to cold |
| 9088 | 4.719033879 | up | 0.0000236 | 0.000252459 | AW620238 | 403 | 23 | 36 | 63 | 1e−07 | |
| 9089 | 4.709847774 | up | 0.000000039 | 0.00000518 | BE823095 | 543 | 28 | 56 | 50 | 2e−07 | protein amino acid dephosphorylation |
| 9090 | 4.705408497 | up | 0.00000087 | 0.000031 | BG405072 | 487 | 34 | 53 | 64 | 1e−14 | |
| 9091 | 4.687851636 | up | 6.66E−08 | 0.00000711 | BE658223 | 884 | 11 | 24 | 45 | 8.7 | proton transport |
| 9092 | 4.685654371 | up | 0.000000114 | 0.00000929 | BI322039 | 422 | 12 | 31 | 38 | 1.4 | |
| 9093 | 4.674492254 | up | 0.000237792 | 0.001279986 | BU080770 | 515 | 51 | 140 | 36 | 6e−19 | |
| 9094 | 4.670948285 | up | 0.0000005 | 0.0000222 | BI971315 | 780 | 14 | 53 | 26 | 5.5 | electron transport |
| 9095 | 4.62590972 | up | 0.00000922 | 0.000132419 | AF004807.1 | | | | | | dehydrin |
| 9096 | 4.613841916 | up | 0.000000112 | 0.0000092 | BE611723 | 438 | 16 | 23 | 69 | 0.002 | |
| 9097 | 4.586231752 | up | 0.000329469 | 0.0016233 | BQ296209 | | | | | | |
| 9098 | 4.572101318 | up | 0.0000113 | 0.000152726 | AI960326 | 233 | 11 | 33 | 33 | 3.2 | |
| 9099 | 4.566934051 | up | 5.73E−08 | 0.00000658 | CD411143 | 681 | 79 | 152 | 51 | 6e−32 | |
| 9100 | 4.529161891 | up | 0.000000172 | 0.0000118 | BI971030 | 793 | 85 | 136 | 62 | 1e−47 | |
| 9101 | 4.527244968 | up | 0.000197028 | 0.001115274 | AW350922 | 761 | 119 | 190 | 62 | 3e−63 | metabolism |
| 9102 | 4.492981093 | up | 0.000066 | 0.000513447 | CF808510 | 753 | 161 | 219 | 73 | 7e−95 | |
| 9103 | 4.490820535 | up | 0.00000276 | 0.0000622 | BQ298213 | 448 | 29 | 43 | 67 | 2e−11 | |
| 9104 | 4.487767259 | up | 0.00000118 | 0.0000363 | BE823976 | 524 | 40 | 108 | 37 | 5e−16 | |
| 9105 | 4.475508096 | up | 0.00000285 | 0.0000636 | AI748644 | 645 | 43 | 50 | 86 | 8e−22 | regulation of transcription\, DNA- |
| | | | | | | | | | | | dependent |
| 9106 | 4.467463826 | up | 0.005897907 | 0.012883669 | BM732585 | 524 | 80 | 173 | 46 | 5e−36 | |
| 9107 | 4.46560931 | up | 0.000000774 | 0.0000291 | BU549148 | 688 | 74 | 99 | 74 | 3e−40 | proteolysis and peptidolysis |
| 9108 | 4.456159172 | up | 0.00000811 | 0.000121601 | BM526951 | 557 | 91 | 128 | 71 | 1e−45 | |
| 9109 | 4.455277415 | up | 0.0000137 | 0.000174178 | BM092330 | 305 | 13 | 40 | 32 | 1.4 | |
| 9110 | 4.436620089 | up | 0.000137557 | 0.000863927 | BU550013 | 528 | 41 | 71 | 57 | 1e−16 | electron transport |
| 9111 | 4.431880179 | up | 0.00012204 | 0.000794604 | CD408166 | 337 | 32 | 54 | 59 | 1e−13 | |
| 9112 | 4.427814907 | up | 0.000363639 | 0.001741746 | BQ610190 | 367 | 11 | 19 | 57 | 6.1 | regulation of transcription |
| 9113 | 4.421307025 | up | 0.000305624 | 0.001538508 | CF809203 | 407 | 75 | 100 | 75 | 8e−40 | Win gene encoding wound-induced |
| | | | | | | | | | | | protein, response to virus |
| 9114 | 4.420722819 | up | 0.00010524 | 0.00071209 | BF325304 | 424 | 17 | 27 | 62 | 6e−04 | |
| 9115 | 4.418644297 | up | 0.0000208 | 0.000230646 | BQ081355 | 405 | 33 | 55 | 60 | 2e−15 | metabolism |
| 9116 | 4.409003862 | up | 0.000000287 | 0.0000158 | BE022791 | 478 | 18 | 22 | 81 | 5e−06 | glycolysis |
| 9117 | 4.402672916 | up | 0.000000352 | 0.0000179 | BU546534 | 606 | 12 | 26 | 46 | 1.3 | leaf morphogenesis |
| 9118 | 4.400103907 | up | 0.000667538 | 0.002713764 | CF806487 | 376 | 46 | 92 | 50 | 9e−21 | |
| 9119 | 4.365725957 | up | 0.0000042 | 0.0000813 | CA802310 | 430 | 38 | 76 | 50 | 8e−20 | |
| 9120 | 4.363803093 | up | 0.001800061 | 0.005551605 | CF808986 | 575 | 84 | 134 | 62 | 1e−43 | |
| 9121 | 4.344211773 | up | 0.018684275 | 0.030830898 | CD408184 | 700 | 14 | 37 | 37 | 1.2 | response to water deprivation |
| 9122 | 4.338961082 | up | 0.000000123 | 0.00000976 | BE823563 | 644 | 69 | 135 | 51 | 2e−34 | |
| 9123 | 4.319896423 | up | 0.000000262 | 0.0000149 | CD397802 | 419 | 14 | 30 | 46 | 1.7 | tRNA modification |
| 9124 | 4.312731431 | up | 0.0000542 | 0.000447122 | AW758966 | 574 | 67 | 113 | 59 | 4e−35 | antibiotic biosynthesis |
| 9125 | 4.309548178 | up | 0.0000112 | 0.000151484 | BM520182 | 476 | 71 | 90 | 78 | 9e−37 | |
| 9126 | 4.300719246 | up | 0.000108891 | 0.000729966 | CF808829 | 668 | 33 | 116 | 28 | 0.001 | |
| 9127 | 4.2971288 | up | 0.00421249 | 0.010149155 | CF808091 | 222 | 37 | 69 | 53 | 3e−13 | |
| 9128 | 4.296363661 | up | 0.000213421 | 0.00118314 | AW156573 | 370 | 12 | 29 | 41 | 1.7 | protein amino acid phosphorylation |
| 9129 | 4.285731492 | up | 0.00000014 | 0.0000106 | BU548355 | 638 | 95 | 139 | 68 | 3e−49 | |
| 9130 | 4.273987309 | up | 0.0000751 | 0.000561991 | AW569822 | 505 | 134 | 166 | 80 | 2e−80 | sucrose metabolism |
| 9131 | 4.219303288 | up | 0.000160214 | 0.000963153 | X60043.1 | | | | | | hypothetical LOC547915 |
| 9132 | 4.209232044 | up | 0.000000392 | 0.0000192 | CD394788 | 401 | 65 | 78 | 83 | 3e−33 | |
| 9133 | 4.208965289 | up | 0.0000113 | 0.000152555 | AW309563 | 520 | 11 | 28 | 39 | 8.1 | sucrose transporter |
| 9134 | 4.204238292 | up | 0.00000296 | 0.0000653 | CD401132 | 710 | 91 | 149 | 61 | 2e−45 | transport |
| 9135 | 4.204120785 | up | 0.0000143 | 0.000179941 | CF807846 | 754 | 68 | 206 | 33 | 8e−17 | |
| 9136 | 4.178441758 | up | 0.00000339 | 0.000071 | BG508180 | | | | | | |
| 9137 | 4.171696034 | up | 0.0000261 | 0.00027023 | CF808931 | 684 | 132 | 216 | 61 | 9e−78 | GmGST mRNA for geraniol-inducible |
| | | | | | | | | | | | glutathione S-transferase |
| 9138 | 4.137045108 | up | 0.000000313 | 0.0000167 | BM528129 | 543 | 10 | 37 | 27 | 3.0 | |
| 9139 | 4.129626487 | up | 5.49E−08 | 0.00000643 | BG654067 | 280 | 16 | 49 | 32 | 0.84 | |
| 9140 | 4.126359104 | up | 0.00024899 | 0.001324311 | AW348412 | 646 | 105 | 164 | 64 | 2e−55 | |
| 9141 | 4.1121412 | up | 0.0000151 | 0.000186217 | BQ628998 | 337 | 12 | 22 | 54 | 1.9 | proteolysis and peptidolysis |
| 9142 | 4.098510549 | up | 0.000306174 | 0.001540043 | CF807977 | 539 | 66 | 99 | 66 | 1e−34 | metabolism |
| 9143 | 4.095036536 | up | 0.000543052 | 0.00233818 | BU544413 | 491 | 35 | 75 | 46 | 2e−12 | |
| 9144 | 4.075036554 | up | 0.00000652 | 0.000106503 | BM886777 | 435 | 17 | 33 | 51 | 0.23 | |
| 9145 | 4.037609841 | up | 0.019597726 | 0.03200676 | BM085244 | 559 | 102 | 195 | 52 | 5e−49 | |
| 9146 | 4.037253088 | up | 0.000355635 | 0.001714323 | CD398280 | 363 | 21 | 35 | 60 | 6e−06 | |
| 9147 | 4.028142798 | up | 4.54E−08 | 0.00000565 | BQ627845 | 434 | 16 | 36 | 44 | 0.17 | |
| 9148 | 4.019311482 | up | 0.000347637 | 0.001684804 | BE820574 | 455 | 40 | 59 | 67 | 8e−19 | |
| 9149 | 4.015093364 | up | 0.001030239 | 0.003725388 | BM891960 | 548 | 14 | 44 | 31 | 1.8 | |
| 9150 | 4.012376978 | up | 0.000857119 | 0.003260153 | CD411677 | 623 | 45 | 81 | 55 | 4e−18 | proton transport |
| 9151 | 3.991023086 | up | 0.000000351 | 0.0000178 | CA935681 | 265 | 13 | 29 | 44 | 0.65 | |
| 9152 | 3.989614426 | up | 0.000000236 | 0.0000141 | BE807836 | 536 | 54 | 73 | 73 | 1e−25 | |
| 9153 | 3.986329473 | up | 0.00000144 | 0.000041 | CF807137 | 160 | 11 | 26 | 42 | 4.1 | regulation of transcription\, DNA- |
| | | | | | | | | | | | dependent |
| 9154 | 3.983397377 | up | 0.000000401 | 0.0000194 | CD394008 | 558 | 20 | 66 | 30 | 1.4 | |
| 9155 | 3.982597045 | up | 0.00000182 | 0.0000479 | AW201449 | 579 | 67 | 115 | 58 | 3e−36 | fatty acid biosynthesis |
| 9156 | 3.97868573 | up | 0.0000228 | 0.000246214 | BI970404 | 796 | 66 | 102 | 64 | 2e−28 | |
| 9157 | 3.970363272 | up | 0.001448418 | 0.004754826 | BQ473598 | 447 | 27 | 58 | 46 | 3e−06 | |
| 9158 | 3.965025711 | up | 0.000000145 | 0.0000108 | BM085488 | 564 | 154 | 189 | 81 | 1e−88 | |
| 9159 | 3.962647672 | up | 0.001433918 | 0.00472068 | BQ473598 | 447 | 27 | 58 | 46 | 3e−06 | |
| 9160 | 3.960362362 | up | 0.00000652 | 0.000106503 | AW706751 | 300 | 38 | 95 | 40 | 5e−07 | |
| 9161 | 3.947963073 | up | 0.00000306 | 0.0000667 | BE658150 | 865 | 94 | 184 | 51 | 9e−42 | metabolism |
| 9162 | 3.946361742 | up | 0.00073493 | 0.002903746 | BE658456 | 820 | 106 | 234 | 45 | 3e−49 | |
| 9163 | 3.94098642 | up | 0.000134988 | 0.000852568 | AW570011 | 478 | 125 | 159 | 78 | 3e−72 | protein folding |
| 9164 | 3.934675842 | up | 0.0000217 | 0.000237277 | BM084952 | 420 | 17 | 59 | 28 | 0.35 | protein amino acid phosphorylation |
| 9165 | 3.934508918 | up | 0.000461962 | 0.002076044 | AW396425 | 361 | 11 | 25 | 44 | 0.40 | |
| 9166 | 3.674569176 | up | 0.000000595 | 0.0000247 | BE658217 | 879 | 62 | 86 | 72 | 4e−35 | nucleotide-excision repair |
| 9167 | 3.673833221 | up | 0.000814134 | 0.003135981 | CF805987 | 700 | 19 | 19 | 100 | 0.001 | ubiquitin-dependent protein catabolism |
| 9168 | 3.673284152 | up | 0.0000554 | 0.000454382 | AF529303.1 | | | | | | PR10-like protein |
| 9169 | 3.672801228 | up | 0.000000144 | 0.0000108 | BU544330 | 461 | 48 | 118 | 40 | 9e−18 | protein amino acid phosphorylation |
| 9170 | 3.648312769 | up | 0.00000338 | 0.0000709 | CD395251 | 598 | 18 | 23 | 78 | 4e−04 | |
| 9171 | 3.645641975 | up | 0.000000259 | 0.0000148 | CF806499 | 369 | 40 | 109 | 36 | 4e−09 | |
| 9172 | 3.914428073 | up | 0.0000115 | 0.00015437 | CA820071 | 427 | 25 | 44 | 56 | 3e−11 | glutathione transferase, aromatic amino |
| | | | | | | | | | | | acid family metabolism |
| 9173 | 3.899485945 | up | 0.000401197 | 0.001872751 | AW351024 | 804 | 17 | 51 | 33 | 0.90 | |
| 9174 | 3.893915841 | up | 0.000449096 | 0.002030324 | BU544997 | 376 | 37 | 85 | 43 | 5e−11 | |
| 9175 | 3.888332386 | up | 0.019684223 | 0.032124364 | CF806816 | 660 | 139 | 210 | 66 | 2e−80 | response to pathogenic bacteria |
| | | | | | | | | | | | (incompatible interaction) |
| 9176 | 3.886293829 | up | 0.00000123 | 0.0000372 | BG405072 | 487 | 34 | 53 | 64 | 1e−14 | |
| 9177 | 3.882343439 | up | 0.0000021 | 0.0000527 | BM520466 | 304 | 16 | 49 | 32 | 2.5 | |
| 9178 | 3.880231783 | up | 0.00000456 | 0.0000855 | BF595839 | 296 | 22 | 62 | 35 | 0.13 | electron transport |
| 9179 | 3.877286183 | up | 0.000643807 | 0.00264455 | CF806221 | 492 | 131 | 157 | 83 | 9e−78 | |
| 9180 | 3.877260106 | up | 0.000000335 | 0.0000173 | BI095213 | 403 | 47 | 92 | 51 | 6e−21 | transport |
| 9181 | 3.852101476 | up | 0.005463888 | 0.012185877 | BG882930 | 514 | 84 | 130 | 64 | 5e−44 | metabolism |
| 9182 | 3.848775223 | up | 0.000270751 | 0.001406438 | BM954809 | 552 | 64 | 70 | 91 | 3e−33 | proline biosynthesis |
| 9183 | 3.828015972 | up | 0.009585407 | 0.018356336 | AI856683 | 340 | 15 | 31 | 48 | 0.005 | proteolysis and peptidolysis |
| 9184 | 3.819270287 | up | 0.001293514 | 0.004376957 | BU549170 | 701 | 41 | 75 | 54 | 2e−13 | |
| 9185 | 3.807427056 | up | 0.00000361 | 0.0000738 | AW568454 | 521 | 13 | 25 | 52 | 0.051 | protein modification |
| 9186 | 3.80622894 | up | 0.00032701 | 0.001614542 | BI321106 | 381 | 24 | 34 | 70 | 5e−11 | |
| 9187 | 3.796770894 | up | 0.000452218 | 0.002042231 | BU547713 | 570 | 123 | 164 | 75 | 3e−60 | trehalose biosynthesis |
| 9188 | 3.767169249 | up | 0.000586992 | 0.002475792 | BG157182 | 490 | 17 | 27 | 62 | 0.015 | |
| 9189 | 3.748803795 | up | 0.0000263 | 0.000272167 | BI471538 | 372 | 15 | 43 | 34 | 0.014 | |
| 9190 | 3.744199669 | up | 0.000000782 | 0.0000291 | AW311627 | 232 | 12 | 29 | 41 | 2.4 | carbohydrate metabolism |
| 9191 | 3.739425601 | up | 0.000000513 | 0.0000226 | AW350518 | 720 | 101 | 177 | 57 | 7e−53 | carbohydrate metabolism |
| 9192 | 3.735859421 | up | 0.0000426 | 0.000380036 | BM528903 | 489 | 102 | 133 | 76 | 3e−57 | |
| 9193 | 3.718452046 | up | 0.000000929 | 0.0000323 | CA802347 | 404 | 15 | 30 | 50 | 0.029 | |
| 9194 | 3.71795694 | up | 0.00000118 | 0.0000364 | BE346999 | 672 | 118 | 184 | 64 | 2e−65 | |
| 9195 | 3.705867191 | up | 0.000000542 | 0.0000234 | BE822453 | 408 | 10 | 28 | 35 | 8.2 | regulation of transcription\, DNA- |
| | | | | | | | | | | | dependent |
| 9196 | 3.7009141 | up | 0.00000338 | 0.0000709 | BU550613 | 638 | 19 | 38 | 50 | 1e−04 | response to abscisic acid stimulus |
| 9197 | 3.690124081 | up | 0.001645266 | 0.005221827 | BM886782 | 535 | 50 | 59 | 84 | 3e−24 | electron transport |
| 9198 | 3.687933129 | up | 0.0000584 | 0.000471417 | BI969641 | 777 | 97 | 163 | 59 | 2e−55 | |
| 9199 | 3.47527917 | up | 0.006329009 | 0.013545339 | BM085020 | 569 | 165 | 189 | 87 | 6e−96 | sucrose metabolism |
| 9200 | 3.466044368 | up | 0.00000572 | 0.0000985 | CK606100 | 650 | 76 | 142 | 53 | 6e−31 | |
| 9201 | 3.460476569 | up | 0.00000584 | 0.0000997 | CF808191 | 695 | 169 | 202 | 83 | 8e−95 | transport |
| 9202 | 3.459904289 | up | 0.00000299 | 0.0000658 | CF808908 | 333 | 9 | 21 | 42 | 5.5 | |
| 9203 | 3.458923966 | up | 0.00000084 | 0.0000304 | BU550205 | 555 | 14 | 48 | 29 | 0.098 | |
| 9204 | 3.456289173 | up | 0.001353274 | 0.004523187 | AF048978.1 | | | | | | 2,4-D inducible glutathione S- |
| | | | | | | | | | | | transferase |
| 9205 | 3.450036541 | up | 0.000000151 | 0.000011 | BU764231 | 261 | 15 | 43 | 34 | 2.5 | |
| 9206 | 3.396151503 | up | 0.00000106 | 0.0000344 | CA801749 | 617 | 78 | 141 | 55 | 2e−38 | |
| 9207 | 3.391763415 | up | 0.000108841 | 0.000729806 | BE611571 | 544 | 31 | 50 | 62 | 3e−11 | amino acid transport |
| 9208 | 3.382687176 | up | 0.00000241 | 0.0000576 | BI787366 | 431 | 13 | 38 | 34 | 1.9 | src1 protein |
| 9209 | 3.381670058 | up | 0.00000246 | 0.0000583 | BU578967 | 444 | 12 | 17 | 70 | 1.5 | nitrate assimilation |
| 9210 | 3.381603654 | up | 0.000635812 | 0.002621858 | BQ452792 | 254 | 13 | 32 | 40 | 2.5 | |
| 9211 | 3.447120631 | up | 0.0000968 | 0.000674364 | BU548315 | 543 | 70 | 155 | 45 | 6e−31 | |
| 9212 | 3.44525728 | up | 0.001707865 | 0.005360298 | BI974673 | 388 | 52 | 85 | 61 | 1e−29 | metabolism |
| 9213 | 3.442221332 | up | 0.00000238 | 0.0000571 | CD406120 | 279 | 10 | 23 | 43 | 4.2 | |
| 9214 | 3.634825483 | up | 0.00000707 | 0.000111927 | CD397802 | 419 | 14 | 30 | 46 | 1.7 | tRNA modification |
| 9215 | 3.631703054 | up | 0.0000273 | 0.000278842 | AI522887 | 489 | 14 | 51 | 27 | 0.65 | regulation of transcription\, DNA- |
| | | | | | | | | | | | dependent |
| 9216 | 3.630384639 | up | 0.000000472 | 0.0000216 | BE348000 | 622 | 85 | 197 | 43 | 1e−36 | protein amino acid dephosphorylation |
| 9217 | 3.623916851 | up | 0.00000701 | 0.000111286 | BU549226 | 640 | 27 | 57 | 47 | 6e−09 | metallothionein type 2, copper ion |
| | | | | | | | | | | | homeostasis |
| 9218 | 3.621071918 | up | 0.00294801 | 0.007871544 | BI894054 | 521 | 85 | 175 | 48 | 3e−39 | |
| 9219 | 3.617740848 | up | 0.000854631 | 0.003252995 | BM886273 | 454 | 65 | 142 | 45 | 1e−22 | |
| 9220 | 3.601097045 | up | 0.00000537 | 0.0000947 | BI968699 | 791 | 157 | 201 | 78 | 3e−90 | proline biosynthesis |
| 9221 | 3.594203144 | up | 0.00000189 | 0.0000492 | CD393748 | 543 | 15 | 54 | 27 | 3.0 | |
| 9222 | 3.591157592 | up | 0.00000109 | 0.000035 | BU090905 | 442 | 98 | 142 | 69 | 1e−55 | carbohydrate metabolism |
| 9223 | 3.590495322 | up | 0.0000138 | 0.000175488 | BM887093 | 544 | 16 | 39 | 41 | 3.0 | RNA dependent DNA replication |
| 9224 | 3.588253966 | up | 0.0000543 | 0.000448051 | BG156741 | 662 | 49 | 101 | 48 | 2e−20 | regulation of transcription\, DNA- |
| | | | | | | | | | | | dependent |
| 9225 | 3.58542003 | up | 0.000000109 | 0.00000912 | CD391920 | 456 | 56 | 66 | 84 | 6e−27 | regulation of transcription |
| 9226 | 3.58451301 | up | 0.001657807 | 0.005248884 | CF806638 | 616 | 122 | 162 | 75 | 1e−64 | |
| 9227 | 3.583168989 | up | 0.0000638 | 0.000501635 | BE823831 | 682 | 66 | 82 | 80 | 5e−34 | embryonic development (sensu |
| | | | | | | | | | | | Magnoliophyta) |
| 9228 | 3.568872849 | up | 0.008972485 | 0.017474813 | BE821266 | 658 | 58 | 174 | 33 | 1e−21 | biosynthesis |
| 9229 | 3.567163178 | up | 0.00000532 | 0.0000943 | BI973761 | 453 | 16 | 26 | 61 | 0.32 | |
| 9230 | 3.554100461 | up | 0.001184869 | 0.004109513 | AW567677 | 526 | 45 | 92 | 48 | 1e−21 | phosphoenolpyruvate-dependent sugar |
| | | | | | | | | | | | phosphotransferase system |
| 9231 | 3.544316392 | up | 0.005009008 | 0.0114416 | CD417321 | 357 | 28 | 43 | 65 | 8e−12 | |
| 9232 | 3.537513345 | up | 0.002631957 | 0.007263808 | CF807685 | 552 | 43 | 64 | 67 | 2e−20 | metabolism |
| 9233 | 3.526110098 | up | 0.0000119 | 0.00015762 | BE823183 | 603 | 61 | 89 | 68 | 1e−31 | |
| 9234 | 3.520022204 | up | 0.0000472 | 0.000407571 | L17319.1 | | | | | | |
| 9235 | 3.518260142 | up | 0.00000644 | 0.000105803 | BE822695 | 678 | 125 | 180 | 69 | 1e−71 | |
| 9236 | 3.51364709 | up | 0.0000109 | 0.000149019 | BM528129 | 543 | 10 | 37 | 27 | 3.0 | |
| 9237 | 3.508338877 | up | 0.00000732 | 0.000114437 | D28876.1 | | | | | | cysteine proteinase |
| 9238 | 3.495178494 | up | 0.000000864 | 0.0000309 | BI974790 | 560 | 42 | 53 | 79 | 5e−20 | regulation of transcription\, DNA- |
| | | | | | | | | | | | dependent |
| 9239 | 3.494909998 | up | 0.0000377 | 0.000347867 | BM887549 | 407 | 63 | 77 | 81 | 2e−34 | cell wall biosynthesis (sensu |
| | | | | | | | | | | | Magnoliophyta) |
| 9240 | 3.491384428 | up | 0.00076219 | 0.002985113 | AW279542 | 473 | 44 | 82 | 53 | 1e−21 | electron transport |
| 9241 | 3.483987456 | up | 0.0000568 | 0.000463339 | BI969348 | 427 | 38 | 63 | 60 | 2e−12 | |
| 9242 | 3.483560951 | up | 0.0000058 | 0.0000992 | CA782606 | 536 | 27 | 76 | 35 | 1e−05 | metal ion transport |
| 9243 | 3.482162633 | up | 0.0002373 | 0.001278766 | CK606097 | 773 | 48 | 155 | 30 | 1e−07 | PR10-like protein |
| 9244 | 3.438435851 | up | 0.0000451 | 0.000394673 | BU547692 | 609 | 16 | 39 | 41 | 0.089 | regulation of transcription |
| 9245 | 3.428712481 | up | 0.000174217 | 0.001023107 | BF598837 | 457 | 42 | 47 | 89 | 2e−20 | proline biosynthesis |
| 9246 | 3.428710449 | up | 0.000000902 | 0.0000318 | BU089704 | 353 | 30 | 47 | 63 | 6e−13 | ciliary/flagellar motility |
| 9247 | 3.423795246 | up | 0.000000254 | 0.0000148 | BI892788 | 291 | 20 | 29 | 68 | 4e−06 | transport |
| 9248 | 3.414808025 | up | 0.000000824 | 0.00003 | BG654181 | 298 | 14 | 39 | 35 | 4.2 | regulation of transcription\, DNA- |
| | | | | | | | | | | | dependent |
| 9249 | 3.373819317 | up | 0.003792558 | 0.009406744 | CF807071 | 518 | 56 | 104 | 53 | 5e−28 | phosphoenolpyruvate-dependent sugar |
| | | | | | | | | | | | phosphotransferase system |
| 9250 | 3.368139756 | up | 0.001751191 | 0.005452756 | BU547713 | 570 | 123 | 164 | 75 | 3e−60 | trehalose biosynthesis |
| 9251 | 3.352561349 | up | 0.017636707 | 0.029438078 | BM954800 | 525 | 59 | 72 | 81 | 1e−29 | carotenoid biosynthesis |
| 9252 | 3.350971785 | up | 0.001917172 | 0.005808498 | BM085620 | 476 | 56 | 101 | 55 | 4e−31 | |
| 9253 | 3.349354882 | up | 0.000162381 | 0.000972253 | BF324893 | 656 | 56 | 113 | 49 | 2e−24 | |
| 9254 | 3.347976266 | up | 0.0000272 | 0.000277579 | CF807343 | 655 | 144 | 198 | 72 | 3e−79 | response to wounding |
| 9255 | 3.34282411 | up | 0.00179782 | 0.005546979 | AI938534 | 481 | 45 | 107 | 42 | 2e−14 | |
| 9256 | 3.342624408 | up | 0.0000594 | 0.000477046 | BI424905 | 354 | 49 | 54 | 90 | 1e−24 | cytosolic glutamine synthetase alpha |
| 9257 | 3.337725925 | up | 0.00000276 | 0.0000622 | BQ740671 | 425 | 37 | 62 | 59 | 3e−16 | |
| 9258 | 3.32831809 | up | 0.0000993 | 0.000684267 | AF117725.1 | | | | | | seed maturation protein PM29 |
| 9259 | 3.311119031 | up | 0.00000082 | 0.0000299 | BM954206 | 498 | 12 | 25 | 48 | 0.68 | regulation of transcription |
| 9260 | 3.308921126 | up | 0.000000334 | 0.0000173 | CF809317 | 380 | 30 | 46 | 65 | 8e−14 | protein folding |
| 9261 | 3.303808641 | up | 0.00016433 | 0.000978955 | BM095066 | 300 | 26 | 45 | 57 | 3e−08 | sucrose transporter, phloem loading |
| 9262 | 3.303064291 | up | 0.00000148 | 0.0000417 | AI748644 | 645 | 43 | 50 | 86 | 8e−22 | regulation of transcription\, DNA- |
| | | | | | | | | | | | dependent |
| 9263 | 3.300113311 | up | 0.001801041 | 0.005553728 | BU545511 | 510 | 37 | 109 | 33 | 4e−14 | |
| 9264 | 3.291690594 | up | 0.032567972 | 0.04888705 | CA801752 | 645 | 108 | 163 | 66 | 4e−54 | transition metal ion homeostasis |
| 9265 | 3.289265088 | up | 0.007223871 | 0.014918123 | BI425441 | 336 | 17 | 22 | 77 | 1e−06 | |
| 9266 | 3.288955604 | up | 0.00000642 | 0.000105761 | BI699405 | 420 | 20 | 50 | 40 | 0.002 | regulation of transcription\, DNA- |
| | | | | | | | | | | | dependent |
| 9267 | 3.286362969 | up | 0.000405981 | 0.001889854 | AW184812 | 481 | 10 | 22 | 45 | 4.0 | |
| 9268 | 3.284299635 | up | 0.0000586 | 0.000472951 | BU546419 | 502 | 32 | 73 | 43 | 2e−09 | |
| 9269 | 3.201197823 | up | 0.000654445 | 0.002675381 | BU549461 | 597 | 86 | 146 | 58 | 6e−51 | electron transport |
| 9270 | 3.20057945 | up | 0.00000138 | 0.00004 | CD416257 | 630 | 17 | 31 | 54 | 0.12 | regulation of transcription\, DNA- |
| | | | | | | | | | | | dependent |
| 9271 | 3.20030384 | up | 0.007452518 | 0.015261271 | BF324224 | 329 | 17 | 38 | 44 | 5.6 | oligopeptide transport |
| 9272 | 3.196734168 | up | 0.00000799 | 0.000120479 | BQ611662 | 393 | 12 | 33 | 36 | 2.0 | |
| 9273 | 3.195551735 | up | 0.000000487 | 0.000022 | AW733977 | 289 | 10 | 30 | 33 | 0.37 | |
| 9274 | 3.277813846 | up | 0.0000287 | 0.000288441 | AW156522 | 219 | 14 | 38 | 36 | 7.2 | |
| 9275 | 3.158662798 | up | 0.001125366 | 0.00396864 | AW707079 | 419 | 37 | 59 | 62 | 4e−14 | gluconeogenesis |
| 9276 | 3.275290713 | up | 0.000000488 | 0.000022 | AW309583 | 516 | 53 | 82 | 64 | 3e−27 | carbohydrate metabolism |
| 9277 | 3.265729529 | up | 0.00000122 | 0.0000371 | AW509040 | 667 | 50 | 142 | 35 | 8e−13 | response to abscisic acid stimulus |
| 9278 | 3.255949299 | up | 0.0000381 | 0.000351187 | BG650581 | 397 | 33 | 51 | 64 | 7e−12 | |
| 9279 | 3.254433424 | up | 0.000589985 | 0.002483682 | CD396847 | 478 | 49 | 69 | 71 | 8e−22 | response to UV |
| 9280 | 3.241693641 | up | 0.00000108 | 0.000035 | CD416257 | 630 | 17 | 31 | 54 | 0.12 | regulation of transcription\, DNA- |
| | | | | | | | | | | | dependent |
| 9281 | 3.240280742 | up | 0.0000696 | 0.000533425 | BE348034 | 660 | 81 | 124 | 65 | 5e−40 | |
| 9282 | 3.239798237 | up | 0.0000199 | 0.000222823 | BM887064 | 552 | 158 | 184 | 85 | 5e−89 | |
| 9283 | 3.236682472 | up | 0.000112785 | 0.000749124 | BF598694 | 482 | 45 | 78 | 57 | 2e−21 | |
| 9284 | 3.231767013 | up | 0.0000639 | 0.000502167 | BU550596 | 480 | 15 | 32 | 46 | 0.81 | |
| 9285 | 3.230861349 | up | 0.00000256 | 0.0000598 | BE823069 | 502 | 73 | 108 | 67 | 3e−37 | amino acid transport |
| 9286 | 3.227682903 | up | 0.0000946 | 0.000662463 | BI786528 | 166 | 23 | 25 | 92 | 9e−09 | |
| 9287 | 3.227393322 | up | 0.00000673 | 0.000108581 | CD397542 | 493 | 33 | 43 | 76 | 3e−13 | |
| 9288 | 3.214049812 | up | 0.000736944 | 0.002910481 | BU548558 | 346 | 19 | 27 | 70 | 9e−06 | |
| 9289 | 3.205050429 | up | 0.000000644 | 0.0000259 | CD415020 | 563 | 31 | 64 | 48 | 7e−08 | regulation of transcription\, DNA- |
| | | | | | | | | | | | dependent |
| 9290 | 3.168191185 | up | 0.00000155 | 0.000043 | AW457857 | 459 | 54 | 104 | 51 | 1e−28 | electron transport |
| 9291 | 3.154470843 | up | 0.000763785 | 0.002990114 | BI700202 | 420 | 14 | 43 | 32 | 5.1 | abscisic acid biosynthesis |
| 9292 | 3.152886254 | up | 0.000000285 | 0.0000157 | CD413163 | 599 | 130 | 177 | 73 | 1e−69 | |
| 9293 | 3.005037748 | up | 0.000102419 | 0.000699165 | BM887549 | 407 | 63 | 77 | 81 | 2e−34 | cell wall biosynthesis (sensu |
| | | | | | | | | | | | Magnoliophyta) |
| 9294 | 3.004426317 | up | 0.00000152 | 0.0000425 | BQ473855 | 577 | 46 | 103 | 44 | 7e−14 | |
| 9295 | 2.999634073 | up | 0.007107009 | 0.014744602 | BQ741076 | 588 | 72 | 93 | 77 | 5e−38 | |
| 9296 | 2.999547879 | up | 0.00000185 | 0.0000483 | BQ079450 | 261 | 10 | 33 | 30 | 1.1 | metabolism |
| 9297 | 2.995597518 | up | 0.000000584 | 0.0000244 | BU545606 | 645 | 52 | 122 | 42 | 5e−17 | protein amino acid phosphorylation |
| 9298 | 2.995305602 | up | 0.00000284 | 0.0000634 | BE824376 | 626 | 53 | 108 | 49 | 4e−27 | |
| 9299 | 2.994926536 | up | 0.005480571 | 0.012209638 | CF807071 | 518 | 56 | 104 | 53 | 5e−28 | phosphoenolpyruvate-dependent sugar |
| | | | | | | | | | | | phosphotransferase system |
| 9300 | 3.147989267 | up | 0.013908936 | 0.024357313 | BU550820 | 636 | 12 | 43 | 27 | 3.1 | |
| 9301 | 3.14402892 | up | 0.000496569 | 0.002187884 | AF004810.1 | | | | | | late-embryogenesis abundant protein |
| 9302 | 3.137220879 | up | 0.000151944 | 0.000924641 | BM568229 | 426 | 56 | 94 | 59 | 7e−29 | cell wall organization and biogenesis |
| | | | | | | | | | | | (sensu Magnoliophyta) |
| 9303 | 3.133374421 | up | 0.00000276 | 0.0000622 | AW597827 | 426 | 44 | 56 | 78 | 2e−17 | DNA replication initiation |
| 9304 | 3.130762258 | up | 0.010158278 | 0.019161198 | BM143872 | 581 | 44 | 113 | 38 | 3e−21 | electron transport |
| 9305 | 3.128423585 | up | 0.0000269 | 0.000275977 | CA802806 | 622 | 89 | 211 | 42 | 5e−35 | protein amino acid dephosphorylation |
| 9306 | 3.123166213 | up | 0.001208521 | 0.00417116 | BG510223 | 281 | 20 | 42 | 47 | 2e−04 | |
| 9307 | 3.118939232 | up | 0.0000497 | 0.000420836 | BG650172 | 416 | 57 | 91 | 62 | 4e−27 | protein biosynthesis |
| 9308 | 3.115870748 | up | 0.000500537 | 0.002199083 | BI788222 | 420 | 38 | 42 | 90 | 2e−17 | response to stress |
| 9309 | 3.111994246 | up | 0.0000113 | 0.000152786 | CD392933 | 564 | 36 | 57 | 63 | 9e−17 | |
| 9310 | 3.079439027 | up | 0.00000216 | 0.0000536 | CD412016 | 668 | 19 | 43 | 44 | 0.67 | |
| 9311 | 3.078345119 | up | 0.000862781 | 0.003275727 | AW761339 | 407 | 38 | 53 | 71 | 8e−19 | |
| 9312 | 3.071084555 | up | 0.001330797 | 0.004468939 | BI320909 | 395 | 15 | 53 | 28 | 2.6 | |
| 9313 | 3.063947029 | up | 0.000763139 | 0.002988295 | AI965726 | 544 | 56 | 79 | 70 | 1e−27 | |
| 9314 | 3.060077752 | up | 0.025041668 | 0.039173104 | CD408184 | 700 | 14 | 37 | 37 | 1.2 | response to water deprivation |
| 9315 | 3.054991286 | up | 0.000224025 | 0.001224701 | CD408609 | 669 | 102 | 157 | 64 | 4e−52 | photosynthesis light harvesting |
| 9316 | 3.054523646 | up | 0.000158495 | 0.000954928 | AW568266 | 563 | 86 | 177 | 48 | 2e−49 | |
| 9317 | 3.033133909 | up | 0.000486013 | 0.002153987 | BM093776 | 404 | 22 | 70 | 31 | 0.72 | |
| 9318 | 3.02057864 | up | 0.001185787 | 0.0041108 | BG507825 | 458 | 13 | 23 | 56 | 0.030 | trehalose biosynthesis |
| 9319 | 3.01918679 | up | 0.00000141 | 0.0000405 | BU578738 | 371 | 14 | 21 | 66 | 0.003 | electron transport |
| 9320 | 3.017514845 | up | 0.000000942 | 0.0000325 | CA784632 | 628 | 37 | 113 | 32 | 1e−04 | |
| 9321 | 2.982799499 | up | 0.019695997 | 0.032139396 | BG509205 | 311 | 13 | 34 | 38 | 3.2 | |
| 9322 | 2.978148804 | up | 0.00000259 | 0.00006 | BM092330 | 305 | 13 | 40 | 32 | 1.4 | |
| 9323 | 2.977932404 | up | 0.006212921 | 0.013367883 | CF805987 | 700 | 19 | 19 | 100 | 0.001 | ubiquitin-dependent protein catabolism |
| 9324 | 2.750706235 | up | 0.00000315 | 0.0000678 | BU547689 | 706 | 100 | 188 | 53 | 1e−50 | metabolism |
| 9325 | 2.625583195 | up | 0.00000343 | 0.0000716 | BE657443 | 790 | 56 | 102 | 54 | 1e−24 | |
| 9326 | 2.622479184 | up | 0.00000906 | 0.000130834 | BM886786 | 540 | 52 | 73 | 71 | 3e−24 | |
| 9327 | 2.620609863 | up | 0.000573941 | 0.002439347 | BG511314 | 473 | 46 | 135 | 34 | 1e−13 | |
| 9328 | 2.975853218 | up | 0.0000112 | 0.000151895 | CD409598 | 701 | 146 | 165 | 88 | 2e−83 | |
| 9329 | 2.971267495 | up | 0.000564544 | 0.002408389 | CD393581 | 604 | 72 | 134 | 53 | 2e−32 | |
| 9330 | 2.970669172 | up | 0.011146065 | 0.020548356 | BE800180 | 491 | 11 | 32 | 34 | 1.1 | |
| 9331 | 2.95994082 | up | 0.000018 | 0.000208796 | BE822368 | 778 | 154 | 201 | 76 | 5e−92 | transport |
| 9332 | 2.957876668 | up | 0.000000334 | 0.0000173 | BM732249 | 420 | 15 | 30 | 50 | 0.12 | proteolysis and peptidolysis |
| 9333 | 2.943037685 | up | 0.000584401 | 0.00246899 | BI969629 | 778 | 14 | 38 | 36 | 0.17 | |
| 9334 | 2.942285613 | up | 0.0000291 | 0.000291381 | BU081727 | 617 | 11 | 32 | 34 | 6.5 | regulation of transcription\, DNA- |
| | | | | | | | | | | | dependent |
| 9335 | 2.936804573 | up | 0.00030534 | 0.001537491 | BU760760 | 281 | 29 | 74 | 39 | 3e−08 | regulation of transcription\, DNA- |
| | | | | | | | | | | | dependent |
| 9336 | 2.933832728 | up | 0.00000243 | 0.0000579 | BE658194 | 800 | 13 | 31 | 41 | 0.89 | |
| 9337 | 2.933278826 | up | 0.0000527 | 0.000438211 | BQ628029 | 366 | 9 | 19 | 47 | 6.1 | |
| 9338 | 2.932162568 | up | 0.022435302 | 0.035778914 | BU761764 | 622 | 134 | 169 | 79 | 4e−81 | cell expansion |
| 9339 | 2.929926405 | up | 0.000000739 | 0.0000283 | BU550492 | 309 | 15 | 36 | 41 | 3.2 | regulation of transcription\, DNA- |
| | | | | | | | | | | | dependent |
| 9340 | 2.929516661 | up | 0.0000525 | 0.000437241 | AW278758 | 490 | 54 | 79 | 68 | 3e−26 | oligopeptide transport |
| 9341 | 2.925928232 | up | 0.00000844 | 0.00012458 | BI427443 | 289 | 16 | 47 | 34 | 4.1 | |
| 9342 | 2.922720194 | up | 0.0000213 | 0.000233968 | BM890886 | 473 | 10 | 38 | 26 | 8.7 | |
| 9343 | 2.919801225 | up | 0.0000164 | 0.000196845 | BM520182 | 476 | 71 | 90 | 78 | 9e−37 | |
| 9344 | 2.917866277 | up | 0.0000273 | 0.00027863 | CD411653 | 645 | 31 | 45 | 68 | 6e−12 | |
| 9345 | 2.914314603 | up | 0.007229193 | 0.014925943 | CA801701 | 626 | 128 | 185 | 69 | 1e−64 | gluconeogenesis |
| 9346 | 2.913016642 | up | 0.000271629 | 0.001409445 | CF807689 | 756 | 185 | 230 | 80 | e−112 | Cytosolic glutamine synthetase alpha |
| 9347 | 2.91248862 | up | 0.002379844 | 0.006758698 | AF243379.1 | | | | | | glutathione S-transferase GST 24 |
| 9348 | 2.908264969 | up | 0.000000885 | 0.0000313 | CF805900 | 601 | 91 | 182 | 50 | 2e−44 | |
| 9349 | 2.9063309 | up | 0.002254754 | 0.006511348 | AW201969 | 493 | 48 | 67 | 71 | 4e−21 | L-lysine catabolism |
| 9350 | 2.898239764 | up | 0.002318001 | 0.006640969 | CD398706 | 484 | 16 | 59 | 27 | 3.1 | |
| 9351 | 2.894006992 | up | 0.00000157 | 0.0000434 | BI942820 | 601 | 166 | 198 | 83 | 2e−90 | |
| 9352 | 2.892844369 | up | 0.006631786 | 0.014019031 | BU550606 | 601 | 122 | 147 | 82 | 4e−62 | response to heat |
| 9353 | 2.882724607 | up | 0.000126709 | 0.000816939 | CA936708 | 414 | 15 | 43 | 34 | 2.9 | |
| 9354 | 2.877219416 | up | 0.0000776 | 0.000574293 | BG835826 | 415 | 35 | 42 | 83 | 2e−14 | metabolism |
| 9355 | 2.87538116 | up | 0.000570558 | 0.002427712 | BI470046 | 524 | 13 | 28 | 46 | 2.8 | |
| 9356 | 2.785086571 | up | 0.000363088 | 0.001740436 | AF243365.1 | | | | | | glutathione S-transferase GST 10 |
| 9357 | 2.855705626 | up | 0.002539186 | 0.007088642 | AW201454 | 599 | 112 | 198 | 56 | 3e−60 | amino acid transport |
| 9358 | 2.851955587 | up | 0.000150532 | 0.000918878 | BI701059 | 350 | 16 | 22 | 72 | 0.001 | electron transport |
| 9359 | 2.847601395 | up | 0.0000015 | 0.0000421 | BI971156 | 715 | 67 | 120 | 55 | 9e−31 | protein amino acid phosphorylation |
| 9360 | 2.847025403 | up | 0.0000298 | 0.000296734 | AI495634 | 462 | 72 | 174 | 41 | 6e−27 | |
| 9361 | 2.845957893 | up | 0.00000116 | 0.0000362 | BG508857 | 216 | 15 | 37 | 40 | 4.2 | protein amino acid phosphorylation |
| 9362 | 2.842159237 | up | 0.00000763 | 0.000117214 | BI426559 | 471 | 106 | 155 | 68 | 3e−60 | |
| 9363 | 2.840475649 | up | 0.000000873 | 0.000031 | AW396745 | 574 | 100 | 143 | 69 | 2e−55 | |
| 9364 | 2.828214621 | up | 0.007379772 | 0.015154849 | AW395303 | 531 | 35 | 51 | 68 | 8e−19 | transcription |
| 9365 | 2.826767914 | up | 0.013759314 | 0.024149261 | BE659080 | 532 | 51 | 75 | 68 | 1e−22 | metabolism |
| 9366 | 2.826615672 | up | 0.0000538 | 0.000444776 | AW704764 | 500 | 90 | 138 | 65 | 2e−41 | |
| 9367 | 2.824936478 | up | 0.001332832 | 0.004473549 | BF325093 | 420 | 16 | 40 | 40 | 0.011 | |
| 9368 | 2.816227161 | up | 0.00000342 | 0.0000715 | BE823084 | 618 | 100 | 128 | 78 | 8e−57 | |
| 9369 | 2.814488813 | up | 0.00047418 | 0.002115504 | BG363704 | 502 | 20 | 65 | 30 | 2.6 | |
| 9370 | 2.811757234 | up | 0.00000118 | 0.0000364 | BE821882 | 533 | 19 | 54 | 35 | 1.3 | |
| 9371 | 2.808487041 | up | 0.00000432 | 0.0000828 | AW349211 | 764 | 62 | 151 | 41 | 5e−24 | amino acid biosynthesis |
| 9372 | 2.806554417 | up | 0.00000449 | 0.0000847 | BU926059 | 613 | 80 | 213 | 37 | 7e−37 | |
| 9373 | 2.805272123 | up | 0.00000662 | 0.000107417 | BE330112 | 674 | 54 | 81 | 66 | 1e−27 | |
| 9374 | 2.804728568 | up | 0.000681537 | 0.002752049 | BI974947 | 553 | 62 | 78 | 79 | 2e−32 | |
| 9375 | 2.80374916 | up | 0.00000646 | 0.000105906 | BG362818 | | | | | | |
| 9376 | 2.803272232 | up | 0.000293653 | 0.001495057 | BM887283 | 474 | 14 | 26 | 53 | 8.7 | |
| 9377 | 2.802441155 | up | 0.009909915 | 0.018820133 | CD404733 | 472 | 27 | 76 | 35 | 0.006 | |
| 9378 | 2.797532572 | up | 0.00000806 | 0.000121099 | BI316203 | 437 | 27 | 39 | 69 | 5e−11 | metabolism |
| 9379 | 2.791993868 | up | 0.0000128 | 0.000166398 | CA782161 | 409 | 25 | 37 | 67 | 5e−08 | |
| 9380 | 2.79044958 | up | 0.00000333 | 0.0000703 | BU548813 | 637 | 21 | 74 | 28 | 0.62 | |
| 9381 | 2.788532549 | up | 0.000512284 | 0.002237727 | BE023275 | 528 | 24 | 93 | 25 | 0.44 | transcription |
| 9382 | 2.775600493 | up | 0.0000151 | 0.000186864 | BM085509 | 563 | 141 | 190 | 74 | 2e−78 | |
| 9383 | 2.774939172 | up | 0.023456398 | 0.037097246 | CF807381 | 572 | 131 | 188 | 69 | 9e−73 | |
| 9384 | 2.773013161 | up | 0.00000291 | 0.0000644 | AB062756.1 | | | | | | ferritin |
| 9385 | 2.769052928 | up | 0.00000121 | 0.0000371 | CA851347 | 384 | 15 | 40 | 37 | 0.82 | |
| 9386 | 2.766421842 | up | 0.0000469 | 0.000405869 | BU548987 | 563 | 43 | 89 | 48 | 9e−19 | |
| 9387 | 2.7657638 | up | 0.000000978 | 0.0000332 | BU550599 | 624 | 22 | 86 | 25 | 1.7 | apoptosis |
| 9388 | 2.764538286 | up | 0.003008259 | 0.007978611 | BI974098 | 423 | 16 | 59 | 27 | 3.0 | |
| 9389 | 2.758155337 | up | 0.000370358 | 0.001765607 | BM732091 | 345 | 16 | 50 | 32 | 0.059 | |
| 9390 | 2.757050952 | up | 0.000549322 | 0.002358427 | CD418350 | 656 | 18 | 37 | 48 | 0.007 | ciliary/flagellar motility |
| 9391 | 2.755940264 | up | 0.00000173 | 0.0000462 | BQ612550 | 271 | 9 | 22 | 40 | 2.5 | cellulose biosynthesis |
| 9392 | 2.754185469 | up | 0.0000155 | 0.000190034 | BE805791 | 427 | 16 | 39 | 41 | 3.1 | |
| 9393 | 2.741657009 | up | 0.0000919 | 0.000649361 | BQ611988 | 279 | 33 | 40 | 82 | 1e−14 | potassium ion transport |
| 9394 | 2.737037741 | up | 0.000183905 | 0.001060139 | BM527139 | 385 | 25 | 78 | 32 | 0.63 | |
| 9395 | 2.736885267 | up | 0.013187775 | 0.023369686 | BG043272 | 488 | 78 | 102 | 76 | 4e−40 | metabolism |
| 9396 | 2.736751386 | up | 0.0000233 | 0.000249858 | AI856277 | 540 | 16 | 47 | 34 | 3.0 | protein amino acid phosphorylation |
| 9397 | 2.726345945 | up | 0.0000501 | 0.00042319 | BE658881 | 604 | 43 | 102 | 42 | 6e−13 | salinity response |
| 9398 | 2.723932505 | up | 0.001992422 | 0.005966946 | BU550101 | 353 | 12 | 18 | 66 | 0.13 | |
| 9399 | 2.707857707 | up | 0.0000486 | 0.000414922 | BE657213 | 452 | 27 | 50 | 54 | 2e−07 | carbohydrate metabolism |
| 9400 | 2.700041186 | up | 0.023306474 | 0.036906905 | BM890881 | 559 | 60 | 84 | 71 | 6e−34 | metabolism |
| 9401 | 2.698954388 | up | 0.025799308 | 0.040131308 | AW350266 | 613 | 69 | 115 | 60 | 9e−34 | beta-amylase, starch catabolism |
| 9402 | 2.68836824 | up | 0.002580562 | 0.007165603 | AI960883 | 224 | 15 | 62 | 24 | 2.5 | |
| 9403 | 2.684032774 | up | 0.000157613 | 0.000950529 | BU090662 | 583 | 112 | 167 | 67 | 2e−58 | sulfate transport |
| 9404 | 2.678234552 | up | 0.0000224 | 0.000243194 | BE659397 | 557 | 21 | 48 | 43 | 0.009 | copper ion homeostasis |
| 9405 | 2.674825198 | up | 0.002512276 | 0.007039914 | CA799429 | 438 | 15 | 57 | 26 | 5.7 | transport |
| 9406 | 2.672874458 | up | 0.0000377 | 0.000348462 | AF022459.1 | | | | | | cytochrome P450 CYP71D10 |
| 9407 | 2.671044078 | up | 0.011139316 | 0.020538934 | CF808574 | 556 | 119 | 146 | 81 | 2e−71 | cell expansion |
| 9408 | 2.670999472 | up | 0.002219235 | 0.006437938 | AI736534 | 763 | 22 | 95 | 23 | 2.4 | regulation of carbohydrate metabolism |
| 9409 | 2.656614436 | up | 0.000581343 | 0.002461073 | AW351155 | 348 | 19 | 41 | 46 | 2e−04 | |
| 9410 | 2.652346206 | up | 0.002932601 | 0.007844321 | BE331452 | 413 | 93 | 117 | 79 | 7e−53 | mercury ion transport |
| 9411 | 2.652182717 | up | 0.014904687 | 0.025723819 | BM525672 | 587 | 93 | 195 | 47 | 1e−46 | |
| 9412 | 2.651423011 | up | 0.0000029 | 0.0000644 | BQ299308 | 390 | 16 | 48 | 33 | 1.1 | transcription from Pol II promoter |
| 9413 | 2.650642725 | up | 0.0000041 | 0.00008 | BU551388 | 565 | 86 | 119 | 72 | 6e−45 | transport |
| 9414 | 2.650569942 | up | 0.000027 | 0.000276542 | CD391964 | 563 | 21 | 54 | 38 | 2e−06 | |
| 9415 | 2.647470197 | up | 0.001761894 | 0.005476564 | BE821877 | 621 | 74 | 144 | 51 | 3e−31 | |
| 9416 | 2.643666885 | up | 0.0000307 | 0.000302808 | CF805853 | 650 | 61 | 96 | 63 | 1e−29 | electron transport |
| 9417 | 2.643225588 | up | 0.000224896 | 0.001228293 | CD400844 | 574 | 14 | 39 | 35 | 4.4 | |
| 9418 | 2.638480182 | up | 0.000319616 | 0.00158887 | BG510367 | 361 | 14 | 41 | 34 | 2.6 | |
| 9419 | 2.634885218 | up | 0.0000527 | 0.000438802 | CF806554 | 728 | 31 | 40 | 77 | 2e−12 | |
| 9420 | 2.627007749 | up | 0.00000625 | 0.000104425 | BU550489 | 623 | 71 | 96 | 73 | 4e−38 | |
| 9421 | 2.626070809 | up | 0.000408279 | 0.001897942 | BG155319 | 454 | 13 | 20 | 65 | | |
| 9422 | 2.610393832 | up | 0.004543205 | 0.01068251 | BU546635 | | | | | | |
| 9423 | 2.610154407 | up | 0.007690331 | 0.015592615 | CD396438 | 638 | 39 | 62 | 62 | 3e−16 | |
| 9424 | 2.60945482 | up | 0.000104393 | 0.00070912 | CF807983 | 582 | 33 | 65 | 50 | 1e−12 | |
| 9425 | 2.551410915 | up | 0.0000694 | 0.00053218 | CF806554 | 728 | 31 | 40 | 77 | 2e−12 | |
| 9426 | 2.548783807 | up | 0.000372812 | 0.001775058 | BM954621 | 556 | 16 | 38 | 42 | 1.1 | |
| 9427 | 2.547015688 | up | 0.0000565 | 0.000461456 | BI427054 | 324 | 11 | 23 | 47 | 0.49 | |
| 9428 | 2.543576535 | up | 0.000949172 | 0.003519188 | AW203289 | 524 | 129 | 180 | 71 | 6e−69 | |
| 9429 | 2.541544358 | up | 0.00034463 | 0.00167321 | BF595970 | 405 | 37 | 60 | 61 | 2e−13 | |
| 9430 | 2.536250053 | up | 0.000202072 | 0.001136823 | BE331387 | 395 | 66 | 86 | 76 | 2e−33 | protein amino acid phosphorylation |
| 9431 | 2.520741731 | up | 0.0000387 | 0.000353675 | BM271324 | 241 | 27 | 39 | 69 | 8e−10 | |
| 9432 | 2.608135638 | up | 0.001457496 | 0.004771716 | CD393125 | 675 | 64 | 85 | 75 | 6e−32 | |
| 9433 | 2.602493516 | up | 0.000103026 | 0.000702032 | BG155364 | | | | | | |
| 9434 | 2.60158701 | up | 0.003029392 | 0.0080174 | BE822500 | 540 | 11 | 35 | 31 | 3.0 | |
| 9435 | 2.601219048 | up | 0.000992287 | 0.003630882 | CK606097 | 773 | 48 | 155 | 30 | 1e−07 | hypothetical LOC547915 |
| 9436 | 2.599408379 | up | 0.000441256 | 0.002005719 | BU546094 | 554 | 19 | 63 | 30 | 2.4 | signal transduction |
| 9437 | 2.598050629 | up | 0.032870776 | 0.049264907 | CF806150 | 524 | 91 | 180 | 50 | 8e−43 | |
| 9438 | 2.595817978 | up | 0.000526175 | 0.002282761 | BM270350 | 372 | 16 | 31 | 51 | 0.34 | |
| 9439 | 2.593425995 | up | 0.0000232 | 0.000249271 | CF805896 | 718 | 116 | 192 | 60 | 9e−63 | carbohydrate metabolism |
| 9440 | 2.58891697 | up | 0.018719217 | 0.030880428 | U21722.1 | | | | | | Hsp23.9 |
| 9441 | 2.588476781 | up | 0.002404294 | 0.006806798 | CF807689 | 756 | 185 | 230 | 80 | e−112 | Cytosolic glutamine synthetase alpha |
| 9442 | 2.58634893 | up | 0.000346602 | 0.001680689 | BE823084 | 618 | 100 | 128 | 78 | 8e−57 | |
| 9443 | 2.581768407 | up | 0.00000448 | 0.0000847 | CD416793 | 378 | 52 | 68 | 76 | 2e−26 | protein folding |
| 9444 | 2.578051999 | up | 0.000445855 | 0.002020349 | CF806578 | 512 | 15 | 17 | 88 | 0.013 | |
| 9445 | 2.574535016 | up | 0.011120481 | 0.020511242 | BF596986 | 550 | 73 | 185 | 39 | 2e−27 | |
| 9446 | 2.570937434 | up | 0.000101758 | 0.000696165 | BE801610 | 360 | 11 | 40 | 27 | 3.4 | |
| 9447 | 2.569587147 | up | 0.0000196 | 0.000221197 | BG882205 | 300 | 16 | 46 | 34 | 3.2 | |
| 9448 | 2.563018901 | up | 0.009106136 | 0.01765965 | BE022898 | 439 | 47 | 77 | 61 | 1e−23 | very-long-chain fatty acid metabolism |
| 9449 | 2.562913214 | up | 0.000224012 | 0.001224701 | BG510367 | 361 | 14 | 41 | 34 | 2.6 | |
| 9450 | 2.56149439 | up | 0.000595384 | 0.002499416 | AI496298 | 398 | 80 | 129 | 62 | 2e−42 | transport |
| 9451 | 2.554564471 | up | 0.0000338 | 0.00032389 | AI442765 | 668 | 82 | 100 | 82 | 4e−44 | regulation of transcription\, DNA- |
| | | | | | | | | | | | dependent |
| 9452 | 2.513790337 | up | 0.00000837 | 0.000124012 | BU551356 | 571 | 23 | 55 | 41 | 1e−05 | |
| 9453 | 2.512564446 | up | 0.0000137 | 0.000174487 | AW597749 | | | | | | |
| 9454 | 2.512030822 | up | 0.001846804 | 0.005653592 | BM093041 | 167 | 16 | 17 | 94 | 0.003 | electron transport |
| 9455 | 2.503085457 | up | 0.0000092 | 0.000132236 | CF807547 | 724 | 183 | 227 | 80 | e−108 | |
| 9456 | 2.501694456 | up | 0.0000422 | 0.000376794 | AW186006 | 530 | 121 | 137 | 88 | 8e−67 | |
| 9457 | 2.498528888 | up | 0.000976129 | 0.003592575 | BE807870 | 459 | 12 | 22 | 54 | 6.3 | |
| 9458 | 2.409548368 | up | 0.007152541 | 0.014813108 | CF808239 | 692 | 207 | 230 | 90 | e−121 | protein amino acid phosphorylation |
| 9459 | 2.408378253 | up | 0.0000129 | 0.00016769 | BI426947 | 293 | 10 | 21 | 47 | 3.3 | |
| 9460 | 2.40605857 | up | 0.00407209 | 0.009889436 | CD409733 | 629 | 56 | 113 | 49 | 5e−30 | proteolysis and peptidolysis |
| 9461 | 2.403570703 | up | 0.004599558 | 0.010780643 | BI786979 | 578 | 138 | 192 | 71 | 1e−81 | |
| 9462 | 2.403292977 | up | 0.005341634 | 0.011975219 | BM891124 | 546 | 53 | 115 | 46 | 2e−20 | response to abscisic acid stimulus |
| 9463 | 2.442265988 | up | 0.000125908 | 0.000813729 | BG789592 | 523 | 61 | 111 | 54 | 2e−27 | |
| 9464 | 2.441651647 | up | 0.0000333 | 0.000320541 | AW310967 | 551 | 25 | 83 | 30 | 6e−07 | ciliary/flagellar motility |
| 9465 | 2.438098634 | up | 0.000749683 | 0.0029481 | CD403339 | 552 | 38 | 41 | 92 | 4e−16 | metabolism |
| 9466 | 2.496080136 | up | 0.0000057 | 0.0000984 | BI317172 | | | | | | |
| 9467 | 2.49018714 | up | 0.00000229 | 0.0000555 | AW755713 | 289 | 13 | 29 | 44 | 0.17 | protein amino acid phosphorylation |
| 9468 | 2.488525964 | up | 0.0000711 | 0.000541154 | BF426144 | 289 | 14 | 41 | 34 | 7.0 | regulation of transcription\, DNA- |
| | | | | | | | | | | | dependent |
| 9469 | 2.484659387 | up | 0.0000444 | 0.000390395 | CA935018 | 261 | 12 | 29 | 41 | 0.50 | |
| 9470 | 2.483593692 | up | 0.0000128 | 0.000166014 | BU764979 | 518 | 38 | 66 | 57 | 2e−16 | two-component signal transduction |
| | | | | | | | | | | | system (phosphorelay) |
| 9471 | 2.481232781 | up | 0.007368978 | 0.015143613 | BU550606 | 601 | 122 | 147 | 82 | 4e−62 | response to heat |
| 9472 | 2.479280956 | up | 0.0000141 | 0.00017769 | CD411627 | 266 | 10 | 34 | 29 | 1.1 | response to virus |
| 9473 | 2.475031191 | up | 0.0000146 | 0.000182135 | BQ628677 | 384 | 8 | 22 | 36 | 9.1 | metabolism |
| 9474 | 2.470884882 | up | 0.0000457 | 0.000399128 | CA937041 | 550 | 21 | 79 | 26 | 0.21 | protein amino acid phosphorylation |
| 9475 | 2.463731736 | up | 0.000758463 | 0.002975163 | BI968513 | 745 | 149 | 223 | 66 | 1e−79 | |
| 9476 | 2.45791585 | up | 0.000162518 | 0.000972568 | BU083077 | 537 | 11 | 36 | 30 | 8.7 | electron transport |
| 9477 | 2.455835363 | up | 0.0000545 | 0.000449238 | BI423828 | 292 | 13 | 36 | 36 | 3.1 | |
| 9478 | 2.453128174 | up | 0.000191989 | 0.001093672 | AW348576 | 650 | 22 | 33 | 66 | 6e−05 | transport |
| 9479 | 2.45278312 | up | 0.0000168 | 0.000199288 | BU545073 | 582 | 36 | 92 | 39 | 2e−08 | |
| 9480 | 2.451892883 | up | 0.000013 | 0.000168041 | BQ630403 | 616 | 13 | 36 | 36 | 0.002 | |
| 9481 | 2.451335346 | up | 0.001021049 | 0.003701045 | AW156801 | 316 | 11 | 32 | 34 | 0.49 | dd15, RNA dependent DNA replication |
| 9482 | 2.450159038 | up | 0.0000459 | 0.00040003 | BE657248 | 769 | 24 | 62 | 38 | 7e−06 | |
| 9483 | 2.44700163 | up | 0.000101177 | 0.000692862 | BU548987 | 563 | 43 | 89 | 48 | 9e−19 | |
| 9484 | 2.437213367 | up | 0.0000075 | 0.000116063 | BE821656 | 761 | 94 | 141 | 66 | 3e−51 | oligopeptide transport |
| 9485 | 2.434279947 | up | 0.000113613 | 0.000752764 | BE802809 | 371 | 13 | 34 | 38 | 5.0 | regulation of transcription\, DNA- |
| | | | | | | | | | | | dependent |
| 9486 | 2.43288977 | up | 0.00000314 | 0.0000678 | BM891503 | 438 | 46 | 79 | 58 | 2e−20 | response to heat |
| 9487 | 2.431866319 | up | 0.0000101 | 0.000141229 | BG653848 | 301 | 19 | 19 | 100 | 2e−06 | electron transport |
| 9488 | 2.368690569 | up | 0.000039 | 0.000355362 | CD418665 | 522 | 30 | 61 | 49 | 9e−10 | |
| 9489 | 2.368646867 | up | 0.00005 | 0.000422709 | BQ611567 | | | | | | |
| 9490 | 2.367968691 | up | 0.00065537 | 0.002677721 | BI943666 | 254 | 13 | 37 | 35 | 1.9 | proteolysis and peptidolysis |
| 9491 | 2.367204124 | up | 0.0000016 | 0.0000439 | BQ628705 | 215 | 11 | 32 | 34 | 3.2 | transport |
| 9492 | 2.366835642 | up | 0.012346436 | 0.022209414 | CD397612 | 666 | 16 | 45 | 35 | 1.9 | |
| 9493 | 2.366434233 | up | 0.000207294 | 0.001157191 | CA953215 | 440 | 21 | 63 | 33 | 0.69 | |
| 9494 | 2.366432418 | up | 0.001002747 | 0.003655842 | CA819954 | 420 | 17 | 54 | 31 | 0.46 | regulation of transcription\, DNA- |
| | | | | | | | | | | | dependent |
| 9495 | 2.365508696 | up | 0.005556786 | 0.012321697 | CF806144 | 476 | 25 | 46 | 54 | 8e−09 | |
| 9496 | 2.362581095 | up | 0.0000131 | 0.000169269 | BI967417 | 779 | 30 | 86 | 34 | 1e−06 | |
| 9497 | 2.361798594 | up | 0.00000782 | 0.000119065 | BF009272 | 423 | 52 | 71 | 73 | 3e−27 | |
| 9498 | 2.361696172 | up | 0.00017052 | 0.001007058 | U77679.1 | | | | | | asparagine synthetase 1 |
| 9499 | 2.425794878 | up | 0.000148949 | 0.00091234 | AW756869 | 441 | 11 | 25 | 44 | 9.9 | signal transduction |
| 9500 | 2.424925658 | up | 0.0000177 | 0.000207038 | CD406082 | 551 | 71 | 99 | 71 | 5e−40 | protein folding |
| 9501 | 2.424829955 | up | 0.0000189 | 0.000215732 | BE608556 | 340 | 13 | 24 | 54 | 0.38 | regulation of transcription\, DNA- |
| | | | | | | | | | | | dependent |
| 9502 | 2.420448765 | up | 0.00000803 | 0.000120883 | CD395363 | 665 | 14 | 37 | 37 | 7.4 | |
| 9503 | 2.420214247 | up | 0.00000791 | 0.000119798 | AF035255.1 | | | | | | catalase |
| 9504 | 2.416060343 | up | 0.0000167 | 0.00019915 | BU762882 | 578 | 63 | 85 | 74 | 8e−31 | sulfate transport |
| 9505 | 2.411125547 | up | 0.0000266 | 0.000273782 | BI701699 | 553 | 100 | 189 | 52 | 1e−51 | protein amino acid phosphorylation |
| 9506 | 2.397552775 | up | 0.000302188 | 0.001525707 | BE819915 | 866 | 147 | 218 | 67 | 2e−79 | aldehyde dehydrogenase family 7 |
| | | | | | | | | | | | member A1, proline biosynthesis |
| 9507 | 2.395278155 | up | 0.0000644 | 0.00050506 | BE820608 | 477 | 41 | 73 | 56 | 1e−21 | oligopeptide transport |
| 9508 | 2.386541364 | up | 0.00133796 | 0.004484587 | CF808749 | 565 | 15 | 44 | 34 | 4.2 | |
| 9509 | 2.315418832 | up | 0.000374068 | 0.001778628 | CD392491 | 343 | 16 | 23 | 69 | 0.13 | |
| 9510 | 2.381495171 | up | 0.03016543 | 0.045820073 | CF806249 | 589 | 83 | 184 | 45 | 2e−42 | polygalacturonase inhibitor protein |
| 9511 | 2.380716235 | up | 0.004461642 | 0.010551412 | BM270077 | 273 | 24 | 45 | 53 | 4e−09 | |
| 9512 | 2.37889542 | up | 0.0000177 | 0.000207083 | BE823008 | 606 | 46 | 98 | 46 | 2e−18 | protein folding |
| 9513 | 2.37813516 | up | 0.001490388 | 0.004850273 | CF807685 | 552 | 43 | 64 | 67 | 2e−20 | metabolism |
| 9514 | 2.374121002 | up | 0.00000919 | 0.000132236 | BM094795 | 563 | 102 | 193 | 52 | 3e−48 | |
| 9515 | 2.37261114 | up | 0.000182086 | 0.001053364 | U12150.1 | | | | | | protease inhibitor |
| 9516 | 2.370328227 | up | 0.001095478 | 0.003896114 | BQ630399 | 593 | 47 | 174 | 27 | 6e−11 | regulation of transcription |
| 9517 | 2.359231862 | up | 0.000398034 | 0.001861713 | AW706991 | 590 | 76 | 88 | 86 | 3e−39 | plasma membrane proton-ATPase, |
| | | | | | | | | | | | cation transport |
| 9518 | 2.358255552 | up | 0.001089155 | 0.003877472 | BM523928 | 452 | 25 | 33 | 75 | 1e−10 | proteolysis and peptidolysis |
| 9519 | 2.354709065 | up | 0.0100528 | 0.019011816 | BF324498 | 575 | 62 | 111 | 55 | 3e−32 | electron transport |
| 9520 | 2.289697722 | up | 0.0000291 | 0.000291143 | BG359378 | 413 | 18 | 53 | 33 | 1.7 | |
| 9521 | 2.344853347 | up | 0.000114813 | 0.000758841 | CD405782 | 480 | 20 | 58 | 34 | 3.1 | transport |
| 9522 | 2.343357503 | up | 0.0000467 | 0.000404992 | BE820682 | 788 | 97 | 140 | 69 | 9e−49 | |
| 9523 | 2.343209395 | up | 0.0000382 | 0.000351614 | BM085191 | 546 | 80 | 103 | 77 | 1e−44 | |
| 9524 | 2.342498158 | up | 0.000100131 | 0.000688671 | AY143660.2 | | | | | | phosphoenolpyruvate carboxylase |
| | | | | | | | | | | | kinase |
| 9525 | 2.342386682 | up | 0.000186712 | 0.001072373 | BE823731 | 520 | 62 | 97 | 63 | 3e−31 | |
| 9526 | 2.342047272 | up | 0.0000379 | 0.000349143 | CD415997 | 416 | 18 | 44 | 40 | 5.1 | electron transport |
| 9527 | 2.341848951 | up | 0.000032 | 0.000311506 | AB083025.1 | | | | | | syringolide-induced protein 19-1-5 |
| 9528 | 2.339815387 | up | 0.00000837 | 0.000124012 | AW186006 | 530 | 121 | 137 | 88 | 8e−67 | |
| 9529 | 2.335254183 | up | 0.001154476 | 0.004034343 | AW311044 | 337 | 14 | 39 | 35 | 0.85 | |
| 9530 | 2.333659224 | up | 0.003427458 | 0.008732348 | CD408801 | 289 | 23 | 80 | 28 | 0.012 | protein amino acid phosphorylation |
| 9531 | 2.333132161 | up | 0.001918173 | 0.005811064 | CF807773 | 732 | 25 | 61 | 40 | 6e−07 | |
| 9532 | 2.331118458 | up | 0.000632743 | 0.002612225 | CF807852 | 650 | 82 | 149 | 55 | 1e−39 | dehydration responsive element binding |
| | | | | | | | | | | | protein, regulation of transcription\, DNA |
| | | | | | | | | | | | dependent |
| 9533 | 2.330605115 | up | 0.001751501 | 0.005453269 | BF219574 | 678 | 65 | 111 | 58 | 6e−29 | |
| 9534 | 2.330062115 | up | 0.00000403 | 0.000079 | BU765934 | 485 | 18 | 29 | 62 | 7e−05 | |
| 9535 | 2.326635448 | up | 0.00000469 | 0.0000871 | BU546757 | 558 | 50 | 63 | 79 | 5e−23 | |
| 9536 | 2.321756099 | up | 0.008130578 | 0.016249269 | CD406278 | 610 | 27 | 96 | 28 | 7e−04 | |
| 9537 | 2.32159252 | up | 0.010019714 | 0.018961076 | BI470205 | 541 | 158 | 180 | 87 | 1e−90 | |
| 9538 | 2.319905281 | up | 0.00000241 | 0.0000576 | AF167986.1 | | | | | | cysteine proteinase |
| 9539 | 2.315146628 | up | 0.006706283 | 0.014133243 | CD408246 | 676 | 159 | 187 | 85 | 5e−94 | |
| 9540 | 2.311497582 | up | 0.002556675 | 0.007121419 | BI701465 | 279 | 23 | 37 | 62 | 7e−08 | |
| 9541 | 2.306614823 | up | 0.000273639 | 0.00141733 | BM892670 | 383 | 22 | 41 | 53 | 7e−08 | transport |
| 9542 | 2.304202757 | up | 0.0000483 | 0.000413458 | CD416359 | 611 | 101 | 107 | 94 | 5e−56 | |
| 9543 | 2.299687041 | up | 0.0000242 | 0.000256666 | AW133420 | 326 | 13 | 27 | 48 | 5.4 | |
| 9544 | 2.299488174 | up | 0.026385852 | 0.04090329 | CF808418 | 122 | 9 | 14 | 64 | 7.2 | |
| 9545 | 2.299314272 | up | 0.002690293 | 0.007377711 | BQ080887 | 326 | 85 | 87 | 97 | 3e−44 | proton transport |
| 9546 | 2.298178968 | up | 0.00169551 | 0.005331458 | BE820726 | 506 | 57 | 76 | 75 | 7e−30 | regulation of transcription\, DNA- |
| | | | | | | | | | | | dependent |
| 9547 | 2.29699448 | up | 0.00036513 | 0.001747552 | BQ298585 | 595 | 20 | 37 | 54 | 4e−05 | |
| 9548 | 2.296734794 | up | 0.000157986 | 0.000952471 | BU083070 | 542 | 44 | 69 | 63 | 3e−20 | |
| 9549 | 2.294905702 | up | 0.0000944 | 0.000661371 | AW704747 | 315 | 72 | 88 | 81 | 6e−41 | protein folding |
| 9550 | 2.292601352 | up | 0.0000617 | 0.000489456 | AW349454 | 744 | 39 | 77 | 50 | 6e−14 | protein amino acid dephosphorylation |
| 9551 | 2.292365127 | up | 0.004798618 | 0.011103298 | CF807742 | 560 | 67 | 78 | 85 | 6e−36 | photosynthesis light harvesting |
| 9552 | 2.2921687 | up | 0.010244064 | 0.019280471 | BE659199 | 560 | 44 | 98 | 44 | 4e−19 | |
| 9553 | 2.290693317 | up | 0.0000678 | 0.000523764 | BI971318 | 741 | 55 | 118 | 46 | 8e−19 | |
| 9554 | 2.287461683 | up | 0.000902387 | 0.003390172 | BE822523 | 429 | 12 | 29 | 41 | 0.64 | wax biosynthesis |
| 9555 | 2.286735917 | up | 0.0000116 | 0.000155047 | AW472132 | 458 | 15 | 36 | 41 | 2.8 | proteolysis and peptidolysis |
| 9556 | 2.286359027 | up | 0.0000497 | 0.000420922 | CD398333 | 669 | 91 | 129 | 70 | 2e−47 | |
| 9557 | 2.284559699 | up | 0.000202567 | 0.001138405 | AF529301.1 | | | | | | hypothetical LOC547648 |
| 9558 | 2.283973094 | up | 0.0000257 | 0.000267502 | BE022997 | 406 | 34 | 50 | 68 | 2e−14 | |
| 9559 | 2.283039664 | up | 0.000328124 | 0.0016185 | AI794795 | 525 | 17 | 41 | 41 | 0.98 | |
| 9560 | 2.282906924 | up | 0.0000292 | 0.000291829 | AW306442 | 444 | 33 | 46 | 71 | 1e−13 | |
| 9561 | 2.282507633 | up | 0.0000977 | 0.000677813 | BU549156 | 647 | 53 | 79 | 67 | 2e−22 | cation transport |
| 9562 | 2.281752432 | up | 0.004858464 | 0.011200338 | BG507821 | 510 | 67 | 73 | 91 | 2e−34 | |
| 9563 | 2.281080331 | up | 0.000437234 | 0.001992019 | BG789592 | 523 | 61 | 111 | 54 | 2e−27 | |
| 9564 | 2.278574574 | up | 0.0000161 | 0.000194761 | CF809317 | 380 | 30 | 46 | 65 | 8e−14 | protein folding |
| 9565 | 2.275456634 | up | 0.0000588 | 0.000474309 | BG237777 | 325 | 11 | 17 | 64 | 1.9 | proteolysis and peptidolysis |
| 9566 | 2.272064582 | up | 0.006114706 | 0.013222868 | BI785710 | 454 | 47 | 160 | 29 | 3e−13 | tricarboxylic acid cycle |
| 9567 | 2.27013926 | up | 0.0000154 | 0.000189375 | BG043550 | 466 | 107 | 140 | 76 | 1e−56 | protein amino acid phosphorylation |
| 9568 | 2.268774356 | up | 0.003779627 | 0.009385147 | BE057763 | 418 | 7 | 15 | 46 | 3.9 | protein folding |
| 9569 | 2.267926784 | up | 0.00000648 | 0.00010628 | BM093748 | 420 | 18 | 18 | 100 | 0.001 | protein amino acid phosphorylation |
| 9570 | 2.267129118 | up | 0.000353975 | 0.001707635 | AW156522 | 219 | 14 | 38 | 36 | 7.2 | |
| 9571 | 2.266869313 | up | 0.0195967 | 0.032006524 | CF806762 | 695 | 150 | 231 | 64 | 8e−85 | |
| 9572 | 2.264084105 | up | 0.0000741 | 0.000556895 | CA785884 | 608 | 71 | 120 | 59 | 6e−32 | amino acid metabolism |
| 9573 | 2.26196886 | up | 0.002235467 | 0.006472566 | CD417013 | 729 | 105 | 128 | 82 | 4e−58 | |
| 9574 | 2.260531852 | up | 0.0000287 | 0.00028877 | BG043541 | 474 | 100 | 141 | 70 | 5e−59 | |
| 9575 | 2.258462268 | up | 0.000906281 | 0.003400385 | CD392933 | 564 | 36 | 57 | 63 | 9e−17 | |
| 9576 | 2.255997708 | up | 0.0000373 | 0.000345182 | Z46953.1 | | | | | | Heat shock transcription factor 34 |
| 9577 | 2.255707412 | up | 0.0000255 | 0.000266283 | BU550467 | 573 | 42 | 69 | 60 | 3e−21 | |
| 9578 | 2.255454045 | up | 0.000227782 | 0.001239553 | CF921774 | 676 | 17 | 50 | 34 | 1.5 | |
| 9579 | 2.255292815 | up | 0.00000634 | 0.000105046 | CD397037 | 408 | 36 | 62 | 58 | 3e−17 | |
| 9580 | 2.24830848 | up | 0.0000283 | 0.000285971 | BI944138 | 344 | 31 | 56 | 55 | 3e−13 | |
| 9581 | 2.246673245 | up | 0.000114866 | 0.000759062 | CF809033 | 697 | 90 | 166 | 54 | 3e−50 | aromatic amino acid family metabolism |
| 9582 | 2.246582485 | up | 0.003347107 | 0.008597615 | BQ740676 | 483 | 49 | 54 | 90 | 9e−24 | |
| 9583 | 2.246242197 | up | 0.006592653 | 0.013964386 | BU544731 | 485 | 54 | 82 | 65 | 2e−26 | 3-methylcrotonyl-CoA carboxylase, |
| | | | | | | | | | | | biotin-carrier domain, leucine catabolism |
| 9584 | 2.245161906 | up | 0.000387185 | 0.001824821 | BU549111 | 665 | 42 | 44 | 95 | 2e−20 | lipid metabolism |
| 9585 | 2.244422181 | up | 0.011178988 | 0.020583228 | BM177095 | 514 | 31 | 52 | 59 | 2e−10 | |
| 9586 | 2.242832905 | up | 0.002843869 | 0.007677849 | CD413483 | 679 | 89 | 195 | 45 | 7e−33 | regulation of transcription |
| 9587 | 2.242648428 | up | 0.000722533 | 0.002868321 | AW396505 | 443 | 101 | 146 | 69 | 1e−52 | protein amino acid dephosphorylation |
| 9588 | 2.237816756 | up | 0.000151764 | 0.000924215 | AW132176 | 125 | 16 | 37 | 43 | 1.9 | |
| 9589 | 2.230214308 | up | 0.0000457 | 0.000398838 | AW310073 | 628 | 44 | 83 | 53 | 6e−17 | |
| 9590 | 2.227021258 | up | 0.000678365 | 0.002744248 | CD408607 | 702 | 96 | 146 | 65 | 1e−46 | |
| 9591 | 2.227010947 | up | 0.002616075 | 0.007235738 | CD391363 | 548 | 31 | 41 | 75 | 2e−12 | |
| 9592 | 2.223185301 | up | 0.000127822 | 0.000821159 | BI971314 | 774 | 98 | 145 | 67 | 3e−55 | antibiotic biosynthesis |
| 9593 | 2.222657378 | up | 0.000005 | 0.0000908 | AW350244 | 762 | 104 | 165 | 63 | 2e−50 | |
| 9594 | 2.214693376 | up | 0.000219116 | 0.001204943 | BI317786 | 517 | 38 | 49 | 77 | 3e−15 | proline biosynthesis |
| 9595 | 2.213794913 | up | 0.0000565 | 0.000461313 | BM528240 | 470 | 12 | 26 | 46 | 6.7 | |
| 9596 | 2.213447192 | up | 0.00019509 | 0.001106471 | BM567837 | | | | | | |
| 9597 | 2.213156088 | up | 0.0000262 | 0.000271615 | CD391852 | 337 | 14 | 47 | 29 | 7.2 | RNA processing |
| 9598 | 2.211416789 | up | 0.000201488 | 0.001134544 | BM954717 | 518 | 19 | 32 | 59 | 6e−08 | |
| 9599 | 2.210529474 | up | 0.000156784 | 0.000946894 | CK606554 | 586 | 22 | 53 | 41 | 0.001 | |
| 9600 | 2.205969205 | up | 0.006423391 | 0.013691096 | CF807121 | 465 | 73 | 93 | 78 | 6e−41 | proteolysis and peptidolysis |
| 9601 | 2.203595457 | up | 0.000434652 | 0.001985258 | AI938073 | 556 | 85 | 123 | 69 | 3e−42 | |
| 9602 | 2.203321115 | up | 0.0000653 | 0.000509616 | BF595187 | 414 | 16 | 38 | 42 | 5.0 | transport |
| 9603 | 2.201226846 | up | 0.001249131 | 0.00427262 | CD400916 | 639 | 58 | 78 | 74 | 4e−32 | |
| 9604 | 2.200097839 | up | 0.000825339 | 0.003167138 | BQ786198 | 582 | 102 | 195 | 52 | 1e−48 | electron transport |
| 9605 | 2.186419316 | up | 0.000730916 | 0.002893053 | BI469662 | 545 | 138 | 181 | 76 | 7e−79 | |
| 9606 | 2.185538529 | up | 0.000643491 | 0.002644105 | CD408252 | | | | | | |
| 9607 | 2.165542316 | up | 0.009337712 | 0.017992887 | AW568729 | 221 | 35 | 74 | 47 | 4e−11 | |
| 9608 | 2.162695617 | up | 0.0000605 | 0.000482878 | CF806291 | 466 | 24 | 53 | 45 | 9e−05 | |
| 9609 | 2.182354578 | up | 0.0000101 | 0.000141229 | U35367.1 | | | | | | arginine decarboxylase |
| 9610 | 2.120189932 | up | 0.000357554 | 0.001720704 | BU548268 | 499 | 14 | 30 | 46 | 1.2 | carbohydrate biosynthesis |
| 9611 | 2.119651471 | up | 0.003767106 | 0.009359193 | CA852842 | 373 | 11 | 38 | 28 | 6.5 | |
| 9612 | 2.119634038 | up | 0.011722815 | 0.021343819 | BM522757 | 253 | 10 | 31 | 32 | 5.6 | |
| 9613 | 2.118893687 | up | 0.000507962 | 0.002222975 | BI971637 | 142 | 10 | 18 | 55 | 1.9 | vitamin B2 biosynthesis |
| 9614 | 2.117936426 | up | 0.007310038 | 0.015051443 | BG653267 | 379 | 8 | 26 | 30 | 8.7 | lipoxygenase-3 |
| 9615 | 2.071967541 | up | 0.0000653 | 0.000509425 | CF808099 | 474 | 10 | 19 | 52 | 3.9 | |
| 9616 | 2.071965364 | up | 0.000331599 | 0.001628722 | CA936907 | 548 | 32 | 57 | 56 | 1e−13 | regulation of transcription |
| 9617 | 2.181069398 | up | 0.00054437 | 0.002342157 | CD415233 | 366 | 56 | 92 | 60 | 7e−28 | transport |
| 9618 | 2.180933052 | up | 0.0000361 | 0.000338395 | AW156279 | 322 | 10 | 23 | 43 | 9.2 | |
| 9619 | 2.178524368 | up | 0.009394565 | 0.018077821 | AW733824 | 530 | 59 | 91 | 64 | 5e−30 | |
| 9620 | 2.176404572 | up | 0.000289195 | 0.001477558 | BG042067 | 520 | 75 | 177 | 42 | 3e−37 | metabolism |
| 9621 | 2.17275979 | up | 0.000061 | 0.000485903 | BM307631 | | | | | | Seed maturation protein PM23 (PM23) |
| 9622 | 2.172524513 | up | 0.0000197 | 0.000221672 | BF324132 | 432 | 13 | 40 | 32 | 1.4 | metabolism |
| 9623 | 2.168552234 | up | 0.0000484 | 0.0004138 | BM085077 | 441 | 48 | 81 | 59 | 2e−21 | glycolysis |
| 9624 | 2.168208927 | up | 0.000138938 | 0.000870133 | BM093489 | 560 | 51 | 57 | 89 | 2e−24 | transcription initiation |
| 9625 | 2.166432024 | up | 0.000486376 | 0.002154073 | CD402509 | 680 | 55 | 90 | 61 | 4e−29 | |
| 9626 | 2.166116993 | up | 0.000148001 | 0.000907859 | BU550276 | | | | | | |
| 9627 | 2.166097533 | up | 0.003058068 | 0.008074846 | BU765709 | 601 | 121 | 193 | 62 | 2e−67 | |
| 9628 | 2.158480243 | up | 0.00201591 | 0.006016443 | CD417266 | 566 | 27 | 95 | 28 | 2e−07 | regulation of transcription |
| 9629 | 2.157157497 | up | 0.000208504 | 0.001162221 | BG156642 | 514 | 12 | 34 | 35 | 3.5 | |
| 9630 | 2.15122622 | up | 0.000764106 | 0.002990435 | BU761090 | 359 | 32 | 44 | 72 | 3e−16 | syringolide-induced protein 19-1-5, cell |
| | | | | | | | | | | | wall organization and biogenesis (sensu |
| | | | | | | | | | | | Magnoliophyta) |
| 9631 | 2.149692515 | up | 0.0000923 | 0.000650878 | BG156308 | 467 | 86 | 116 | 74 | 5e−40 | regulation of transcription |
| 9632 | 2.148425816 | up | 0.02905115 | 0.044387438 | CD406723 | 691 | 89 | 152 | 58 | 7e−49 | |
| 9633 | 2.148261657 | up | 0.000241121 | 0.001293061 | BE657368 | 566 | 20 | 33 | 60 | 2e−04 | |
| 9634 | 2.146395251 | up | 0.0000242 | 0.000256666 | CF808333 | 694 | 148 | 215 | 68 | 2e−83 | potassium ion transport |
| 9635 | 2.145479553 | up | 0.0000928 | 0.000653494 | BI700868 | 420 | 34 | 87 | 39 | 6e−09 | |
| 9636 | 2.145303722 | up | 0.004547217 | 0.010688602 | BM085273 | 424 | 56 | 82 | 68 | 2e−32 | |
| 9637 | 2.145195673 | up | 0.000131215 | 0.000836532 | CA935481 | | | | | | |
| 9638 | 2.144044039 | up | 0.00018264 | 0.001055319 | BE820822 | 738 | 81 | 137 | 59 | 2e−41 | |
| 9639 | 2.143418455 | up | 0.004855351 | 0.011195315 | CF807083 | 548 | 11 | 16 | 68 | 0.81 | |
| 9640 | 2.14148079 | up | 0.005517602 | 0.01226373 | AFFX-r2-Bs- | | | | | | |
| | | | | | dap-3 | | | | | | |
| 9641 | 2.140773912 | up | 0.000386213 | 0.001822483 | AW156339 | 394 | 15 | 53 | 28 | 5.7 | tryptophan catabolism |
| 9642 | 2.092003764 | up | 0.0000297 | 0.000295113 | BM523687 | 433 | 18 | 30 | 60 | 5e−04 | regulation of transcription\, DNA- |
| | | | | | | | | | | | dependent |
| 9643 | 2.139037484 | up | 0.000226883 | 0.001235734 | BE801610 | 360 | 11 | 40 | 27 | 3.4 | |
| 9644 | 2.136880649 | up | 0.001032392 | 0.003731015 | AW349564 | 420 | 21 | 31 | 67 | 1e−06 | electron transport |
| 9645 | 2.135832825 | up | 0.0000292 | 0.00029179 | CF806377 | 490 | 54 | 99 | 54 | 4e−29 | |
| 9646 | 2.133988957 | up | 0.00000887 | 0.000128818 | BI970432 | 787 | 156 | 191 | 81 | 1e−87 | |
| 9647 | 2.13077473 | up | 0.00000681 | 0.000109267 | BQ628923 | 533 | 73 | 91 | 80 | 2e−38 | |
| 9648 | 2.128435668 | up | 0.001559622 | 0.005019133 | BU763145 | 457 | 12 | 29 | 41 | 0.73 | wax biosynthesis |
| 9649 | 2.128253833 | up | 0.000679817 | 0.002748055 | BM523789 | 439 | 21 | 51 | 41 | 1e−04 | |
| 9650 | 2.1228822 | up | 0.000246803 | 0.00131601 | AF243365.1 | | | | | | glutathione S-transferase GST 10 |
| 9651 | 2.12268091 | up | 0.017780698 | 0.029623208 | AW234498 | 594 | 65 | 174 | 37 | 1e−27 | negative regulation of flavonoid |
| | | | | | | | | | | | biosynthesis |
| 9652 | 2.122303074 | up | 0.01681724 | 0.028336857 | BM094403 | 518 | 106 | 174 | 60 | 4e−58 | |
| 9653 | 2.120494278 | up | 0.0000763 | 0.000567701 | BG156412 | 419 | 59 | 83 | 71 | 4e−31 | |
| 9654 | 2.111382718 | up | 0.000306932 | 0.00154303 | CF808612 | 546 | 57 | 112 | 50 | 4e−31 | |
| 9655 | 2.105633688 | up | 0.0000201 | 0.000224994 | AI416728 | 450 | 18 | 64 | 28 | 2.7 | |
| 9656 | 2.101518593 | up | 0.029008567 | 0.044327778 | BM567859 | 434 | 31 | 76 | 40 | 2e−05 | |
| 9657 | 2.099758854 | up | 0.000048 | 0.000412217 | AW100450 | 463 | 11 | 28 | 39 | 6.4 | regulation of transcription\, DNA- |
| | | | | | | | | | | | dependent |
| 9658 | 2.095057812 | up | 0.000100474 | 0.000690016 | CF806973 | 509 | 62 | 117 | 52 | 8e−26 | |
| 9659 | 2.091001393 | up | 0.000216111 | 0.001194131 | AY373033.1 | | | | | | phosphoenolpyruvate-carboxylase |
| | | | | | | | | | | | kinase |
| 9660 | 2.0902236 | up | 0.000412883 | 0.001912819 | BE820792 | 392 | 47 | 77 | 61 | 3e−21 | |
| 9661 | 2.088086331 | up | 0.000582311 | 0.002463507 | AW349326 | 610 | 35 | 67 | 52 | 2e−17 | |
| 9662 | 2.087305814 | up | 0.0000172 | 0.000203076 | BI426372 | 421 | 32 | 69 | 46 | 2e−14 | regulation of transcription\, DNA- |
| | | | | | | | | | | | dependent |
| 9663 | 2.085100748 | up | 0.003139681 | 0.008218229 | BI784829 | 480 | 38 | 75 | 50 | 1e−13 | regulation of transcription |
| 9664 | 2.083486129 | up | 0.0000118 | 0.000157289 | BQ298212 | 604 | 34 | 61 | 55 | 5e−15 | |
| 9665 | 2.083465715 | up | 0.00010694 | 0.000720348 | BE822975 | 650 | 98 | 127 | 77 | 3e−56 | |
| 9666 | 2.08340155 | up | 0.008795831 | 0.017230686 | BI471456 | 420 | 48 | 71 | 67 | 4e−21 | |
| 9667 | 2.082747383 | up | 0.000212519 | 0.001179023 | BQ612599 | | | | | | |
| 9668 | 2.082519952 | up | 0.0000299 | 0.000297083 | CF808689 | 280 | 12 | 29 | 41 | 0.026 | regulation of transcription |
| 9669 | 2.082484167 | up | 0.0000747 | 0.000559647 | Z46951.1 | | | | | | Heat shock transcription factor 29 |
| 9670 | 2.040013226 | up | 0.001154826 | 0.004035077 | BI973509 | 571 | 157 | 190 | 82 | 3e−87 | glycolysis |
| 9671 | 2.038531181 | up | 0.000918795 | 0.003434319 | CD393613 | 583 | 35 | 77 | 45 | 1e−07 | |
| 9672 | 2.036950128 | up | 0.00004 | 0.000361818 | AW100836 | 277 | 7 | 20 | 35 | 9.3 | |
| 9673 | 2.036758382 | up | 0.0000168 | 0.000199326 | AW705548 | 333 | 12 | 33 | 36 | 7.2 | |
| 9674 | 2.035548057 | up | 0.0000302 | 0.00029957 | BU548759 | 686 | 61 | 82 | 74 | 1e−30 | |
| 9675 | 2.035478414 | up | 0.0000806 | 0.000590799 | BE658289 | 687 | 45 | 111 | 40 | 1e−12 | protein amino acid phosphorylation |
| 9676 | 2.081134407 | up | 0.000021 | 0.000231638 | BM891503 | 438 | 46 | 79 | 58 | 2e−20 | response to heat |
| 9677 | 2.080931019 | up | 0.000115584 | 0.000762599 | BU548694 | 621 | 66 | 111 | 59 | 4e−32 | carbohydrate metabolism |
| 9678 | 2.080214406 | up | 0.001155734 | 0.0040373 | BI472192 | 485 | 79 | 103 | 76 | 1e−42 | electron transport |
| 9679 | 2.079671261 | up | 0.000187958 | 0.001077412 | AW132176 | 125 | 16 | 37 | 43 | 1.9 | |
| 9680 | 2.078114379 | up | 0.0000397 | 0.000360025 | AW100836 | 277 | 7 | 20 | 35 | 9.3 | |
| 9681 | 2.076490713 | up | 0.000393036 | 0.001844139 | BI787825 | 511 | 66 | 102 | 64 | 2e−32 | electron transport |
| 9682 | 2.076150865 | up | 0.0000197 | 0.000222096 | BQ611704 | 371 | 42 | 59 | 71 | 6e−20 | biosynthesis |
| 9683 | 2.070220568 | up | 0.00037673 | 0.001787828 | BG239638 | 292 | 19 | 55 | 34 | 0.13 | |
| 9684 | 2.067163175 | up | 0.013972677 | 0.024453584 | BI321165 | 398 | 22 | 58 | 37 | 1e−04 | |
| 9685 | 2.066676016 | up | 0.002472548 | 0.006952412 | BU550575 | 724 | 52 | 89 | 58 | 2e−23 | |
| 9686 | 2.066092442 | up | 0.0000465 | 0.00040343 | AW349304 | 615 | 68 | 82 | 82 | 1e−28 | |
| 9687 | 2.065654109 | up | 0.0000438 | 0.00038769 | AW348577 | 731 | 66 | 110 | 60 | 2e−30 | amino acid biosynthesis |
| 9688 | 2.065291307 | up | 0.00038145 | 0.001805441 | BI321925 | 256 | 13 | 42 | 30 | 1.1 | |
| 9689 | 2.064490269 | up | 0.000109392 | 0.000732537 | BM520182 | 476 | 71 | 90 | 78 | 9e−37 | |
| 9690 | 2.064479145 | up | 0.000446786 | 0.002022882 | BU577475 | 545 | 80 | 113 | 70 | 4e−42 | |
| 9691 | 2.064372243 | up | 0.000130541 | 0.000833237 | AW310696 | 466 | 57 | 77 | 74 | 8e−30 | |
| 9692 | 2.063609655 | up | 0.000144338 | 0.000892359 | CD402574 | 631 | 53 | 151 | 35 | 8e−20 | |
| 9693 | 2.062752221 | up | 0.0000332 | 0.000320067 | BM891102 | 556 | 26 | 72 | 36 | 0.49 | |
| 9694 | 2.059396363 | up | 0.0000382 | 0.000351614 | BF325217 | 627 | 118 | 157 | 75 | 3e−62 | |
| 9695 | 2.057284517 | up | 0.013798107 | 0.024202667 | AI443559 | 488 | 61 | 75 | 81 | 3e−34 | |
| 9696 | 2.056810631 | up | 0.000583376 | 0.002466407 | AW397746 | 325 | 13 | 45 | 28 | 0.64 | |
| 9697 | 2.053652073 | up | 0.000609515 | 0.002539745 | BI699026 | 426 | 18 | 37 | 48 | 1e−06 | |
| 9698 | 2.053486389 | up | 0.006580466 | 0.013946227 | BE440598 | 247 | 23 | 47 | 48 | 1e−08 | |
| 9699 | 2.052849361 | up | 0.000048 | 0.000412016 | AB069959.1 | | | | | | aspartic proteinase 1 |
| 9700 | 2.052295776 | up | 0.017203169 | 0.028851435 | BM568216 | 303 | 12 | 18 | 66 | 2.5 | |
| 9701 | 2.050679612 | up | 0.007723644 | 0.015646119 | AI461152 | 565 | 53 | 81 | 65 | 4e−31 | response to pathogenic bacteria |
| | | | | | | | | | | | (incompatible interaction) |
| 9702 | 2.049316038 | up | 0.000132568 | 0.000842585 | BE824237 | 538 | 94 | 130 | 72 | 1e−50 | |
| 9703 | 2.049118501 | up | 0.012084301 | 0.021848629 | BG238674 | 476 | 88 | 128 | 68 | 9e−45 | |
| 9704 | 2.046665963 | up | 0.0000366 | 0.000341488 | CD398389 | 462 | 58 | 65 | 89 | 3e−28 | transcription initiation |
| 9705 | 2.046488817 | up | 0.0000731 | 0.000551303 | CD405729 | 293 | 8 | 29 | 27 | 7.2 | |
| 9706 | 2.046066288 | up | 0.00011183 | 0.000744423 | AI938055 | 649 | 62 | 87 | 71 | 1e−30 | |
| 9707 | 2.045193702 | up | 0.000232713 | 0.001260732 | BE803955 | 546 | 81 | 159 | 50 | 3e−38 | regulation of transcription\, DNA- |
| | | | | | | | | | | | dependent |
| 9708 | 2.044596796 | up | 0.000218989 | 0.001204584 | BI969034 | 777 | 45 | 155 | 29 | 4e−09 | regulation of proton transport |
| 9709 | 2.043105889 | up | 0.000503437 | 0.002207798 | BU549025 | 654 | 54 | 124 | 43 | 2e−16 | regulation of transcription |
| 9710 | 2.042904661 | up | 0.0000255 | 0.000266334 | BI700618 | 419 | 19 | 33 | 57 | 2e−04 | |
| 9711 | 2.041115373 | up | 0.002607726 | 0.007221307 | BQ741562 | 572 | 101 | 192 | 52 | 1e−45 | regulation of transcription |
| 9712 | 2.03509736 | up | 0.0000391 | 0.000356022 | BU549140 | 550 | 56 | 82 | 68 | 2e−27 | metabolism |
| 9713 | 2.034124575 | up | 0.000493531 | 0.002177026 | BM085940 | | | | | | |
| 9714 | 2.032088977 | up | 0.0000689 | 0.000529873 | CD397348 | 361 | 16 | 21 | 76 | 2e−04 | |
| 9715 | 2.031692918 | up | 0.00945617 | 0.018163843 | CA782711 | 632 | 76 | 99 | 76 | 6e−42 | |
| 9716 | 2.027997045 | up | 0.0000616 | 0.000489332 | BU546734 | 497 | 19 | 25 | 76 | 1e−04 | |
| 9717 | 2.027277969 | up | 0.010077106 | 0.019049164 | BM521278 | 495 | 17 | 50 | 34 | 3.3 | |
| 9718 | 2.026973264 | up | 0.005097796 | 0.011581113 | BM731185 | 494 | 101 | 163 | 61 | 1e−56 | |
| 9719 | 2.025904799 | up | 0.0000245 | 0.000258992 | AW348257 | 802 | 84 | 200 | 42 | 3e−40 | |
| 9720 | 2.025720732 | up | 0.0000654 | 0.000509812 | BQ298426 | 334 | 18 | 45 | 40 | 0.29 | |
| 9721 | 2.025354317 | up | 0.000300705 | 0.001521079 | CA852124 | 392 | 11 | 31 | 35 | 4.4 | metabolism |
| 9722 | 2.024990502 | up | 0.000116601 | 0.000767423 | BM139949 | 176 | 12 | 19 | 63 | 0.85 | dicarboxylic acid transport |
| 9723 | 2.022570215 | up | 0.004968814 | 0.011378962 | CA938632 | 310 | 12 | 39 | 30 | 3.2 | carbohydrate metabolism |
| 9724 | 2.020442234 | up | 0.000331931 | 0.001629927 | BI971637 | 142 | 10 | 18 | 55 | 1.9 | vitamin B2 biosynthesis |
| 9725 | 2.019605584 | up | 0.000708821 | 0.002830618 | AW349049 | 677 | 66 | 97 | 68 | 4e−32 | electron transport |
| 9726 | 2.017144269 | up | 0.0000815 | 0.000595761 | AW507853 | 441 | 13 | 33 | 39 | 2.0 | |
| 9727 | 2.014744529 | up | 0.000175444 | 0.001027266 | BI967466 | 474 | 16 | 42 | 38 | 0.16 | |
| 9728 | 2.014247921 | up | 0.000015 | 0.000185448 | AF243361.1 | | | | | | glutathione S-transferase GST 6 |
| 9729 | 2.013085219 | up | 0.0000369 | 0.00034315 | BU549224 | 625 | 25 | 70 | 35 | 2e−09 | |
| 9730 | 2.011416845 | up | 0.000155591 | 0.000942414 | BQ273151 | 463 | 11 | 52 | 21 | 0.20 | |
| 9731 | 2.011251831 | up | 0.001818883 | 0.005594526 | BE804860 | 626 | 78 | 94 | 82 | 2e−38 | |
| 9732 | 2.011180324 | up | 0.000714273 | 0.002844538 | BI967949 | 794 | 52 | 77 | 67 | 2e−25 | proline biosynthesis |
| 9733 | 2.010099702 | up | 0.0000488 | 0.0004163 | CD395348 | 426 | 40 | 73 | 54 | 2e−19 | regulation of transcription\, DNA- |
| | | | | | | | | | | | dependent |
| 9734 | 2.009186169 | up | 0.000284193 | 0.001458936 | CK606623 | 632 | 54 | 95 | 56 | 3e−26 | nucleosome assembly |
| 9735 | 2.008071962 | up | 0.0000574 | 0.000466469 | BE824265 | 692 | 54 | 98 | 55 | 2e−19 | protein amino acid dephosphorylation |
| 9736 | 2.007015122 | up | 0.0000687 | 0.000528513 | AW598240 | 413 | 15 | 44 | 34 | 0.27 | |
| 9737 | 2.005853259 | up | 0.013690938 | 0.024053946 | BM520482 | 557 | 100 | 185 | 54 | 1e−58 | electron transport |
| 9738 | 2.003457134 | up | 0.003776619 | 0.009378961 | BE058464 | 223 | 15 | 47 | 31 | 4.2 | |
| 9739 | 2.002452973 | up | 0.00037253 | 0.001773938 | BI974909 | 552 | 43 | 98 | 43 | 1e−11 | |
| 9740 | 2.002442276 | up | 0.013431371 | 0.023698639 | CD396361 | 575 | 23 | 72 | 31 | 0.40 | |
| 9741 | 2.001591851 | up | 0.000273229 | 0.001415598 | BU550552 | 630 | 126 | 159 | 79 | 3e−63 | |
| 9742 | 2.001207194 | up | 0.0000602 | 0.00048128 | CD393748 | 543 | 15 | 54 | 27 | 3.0 | |
| 9743 | 2.001149297 | up | 0.002429785 | 0.006862953 | CD400916 | 639 | 58 | 78 | 74 | 4e−32 | |
| 9744 | 2.000879397 | up | 0.000309821 | 0.001554438 | CF809170 | 528 | 61 | 112 | 54 | 9e−31 | |

### Example 2 Identification of Transcription Factors that are Upregulated in Response to Drought Conditions

Based on database mining of transcription factors, domain homology analysis, and the soybean microarray data obtained in Example 1 using drought-treated root tissues from greenhouse-grown plants, 199 candidate transcription factor genes or ESTs derived from these genes with putative function for drought tolerance were identified. 64 of the candidates showed high sequence similarity to known transcription factor domains and might possess high potential for drought tolerant gene identification. The remaining 135 of the candidates showed relatively low sequence similarity to known transcription factors domains and thus might represent a valuable resource for the identification of novel genes of drought tolerance. The candidates generally belonged to the NAM, zinc finger, bHLH, MYB, AP2, CCAAT-binding, bZIP and WRKY families.

On the basis of family novelty and the magnitude of drought-inducibility, three transcripts were chosen for a pilot experiment to characterize and isolate promoters for drought tolerance studies. The three candidates were BGI56308, BI970909, and BI893889, which belonged to the bHLH, CCAAT-binding, and NAM families, respectively. Under drought condition, the expression levels of these three genes were increased from 2.5 to 252-fold. Moreover, no transcription factor from those families has been reported to control drought tolerance in soybean and other crops. Therefore, these candidate genes may represent novel members of these families that may also play a role in plant drought response. Functional characterization of these transcription factors may help elucidate pathways that are involved in plant drought response.

### Example 3

### Validation of Genes that are Upregulated in Response to Drought Conditions

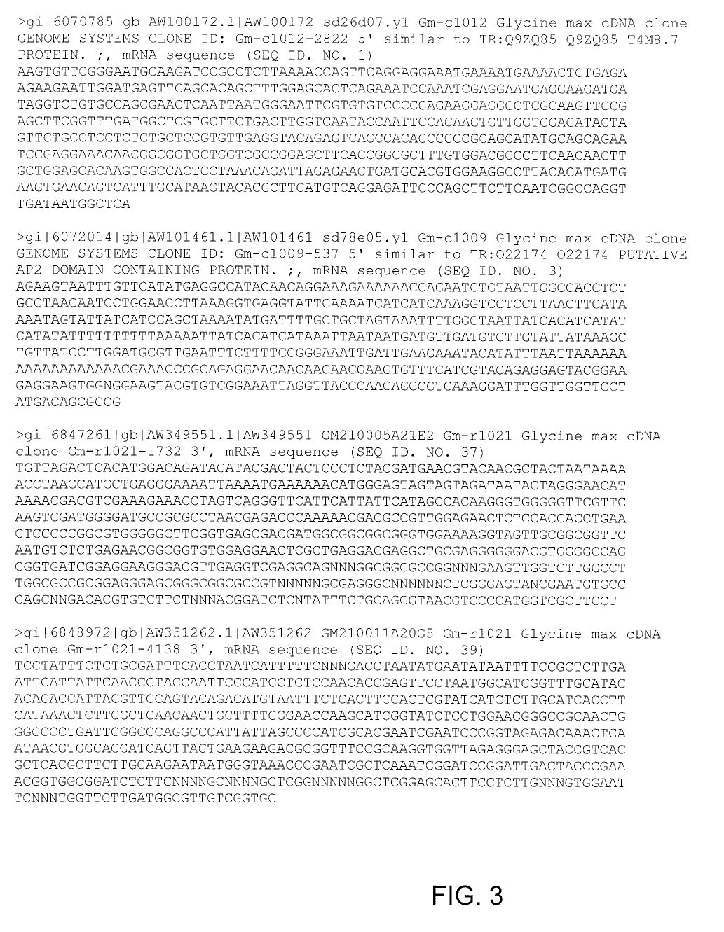

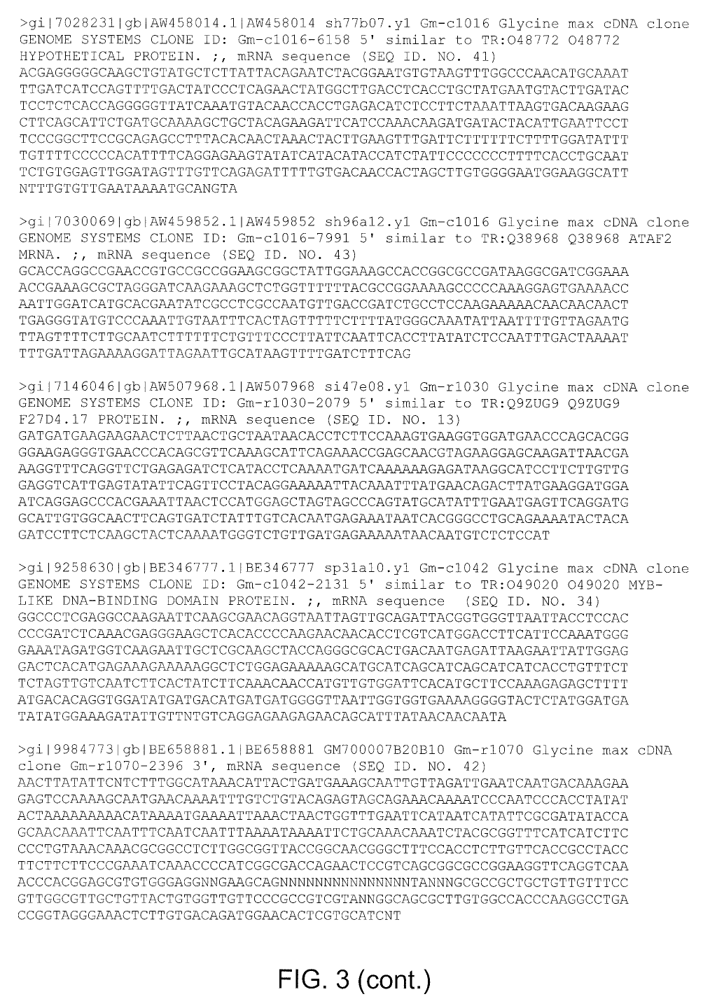

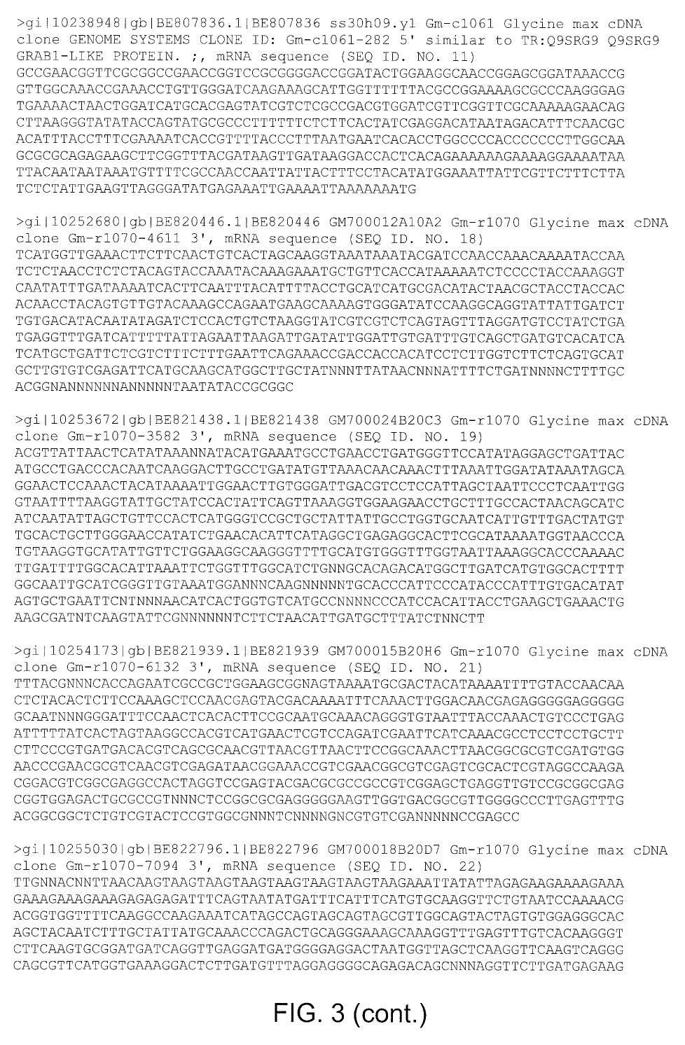

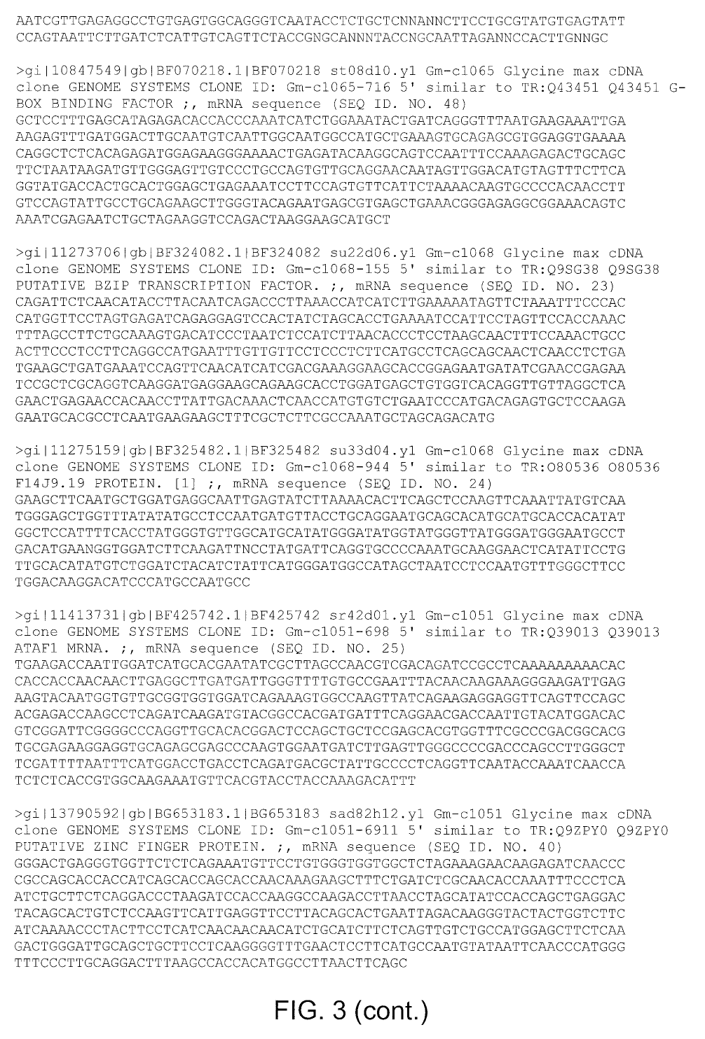

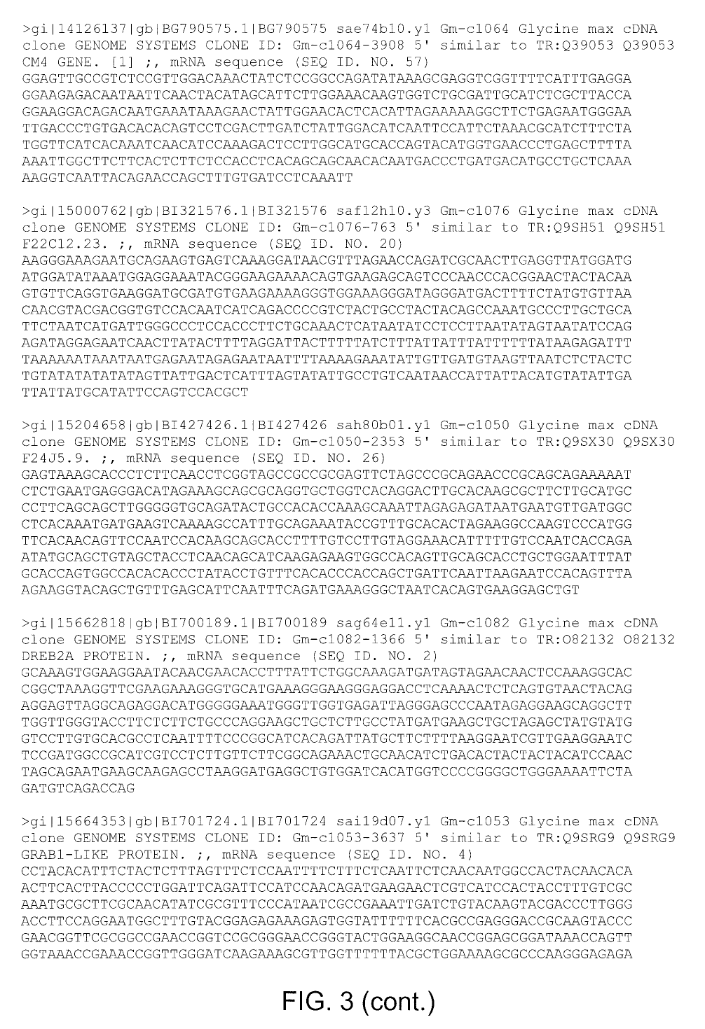

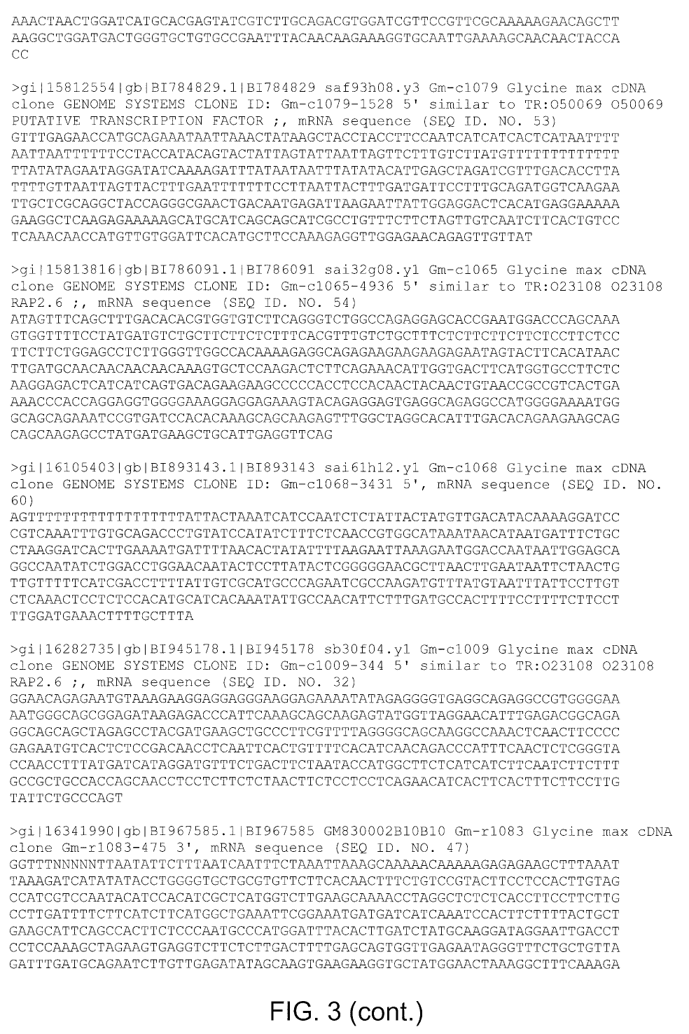

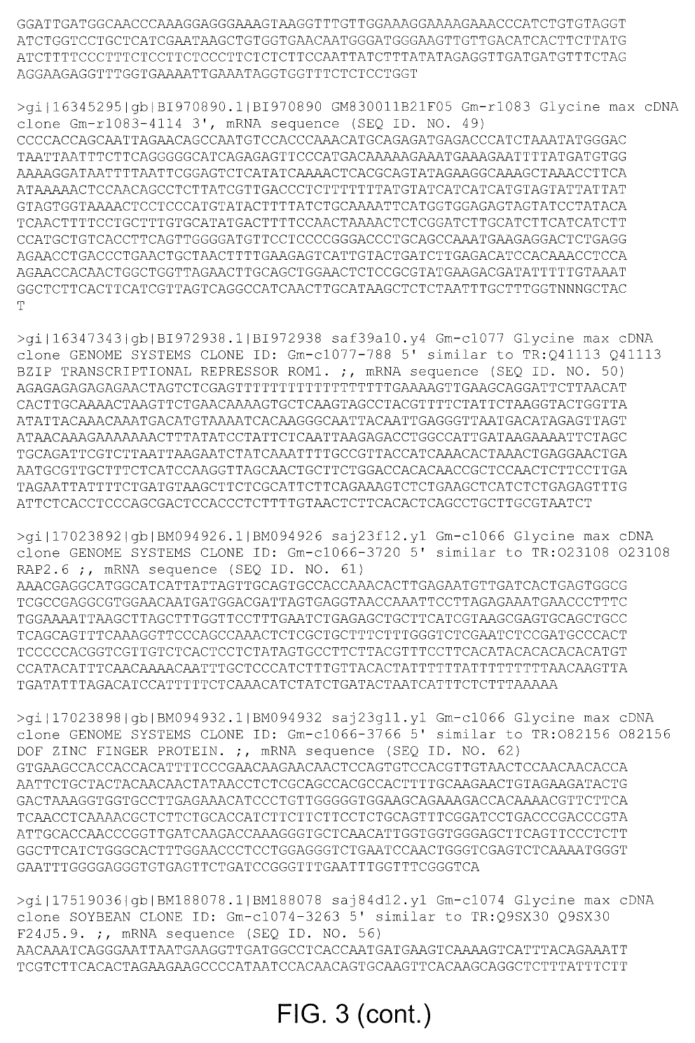

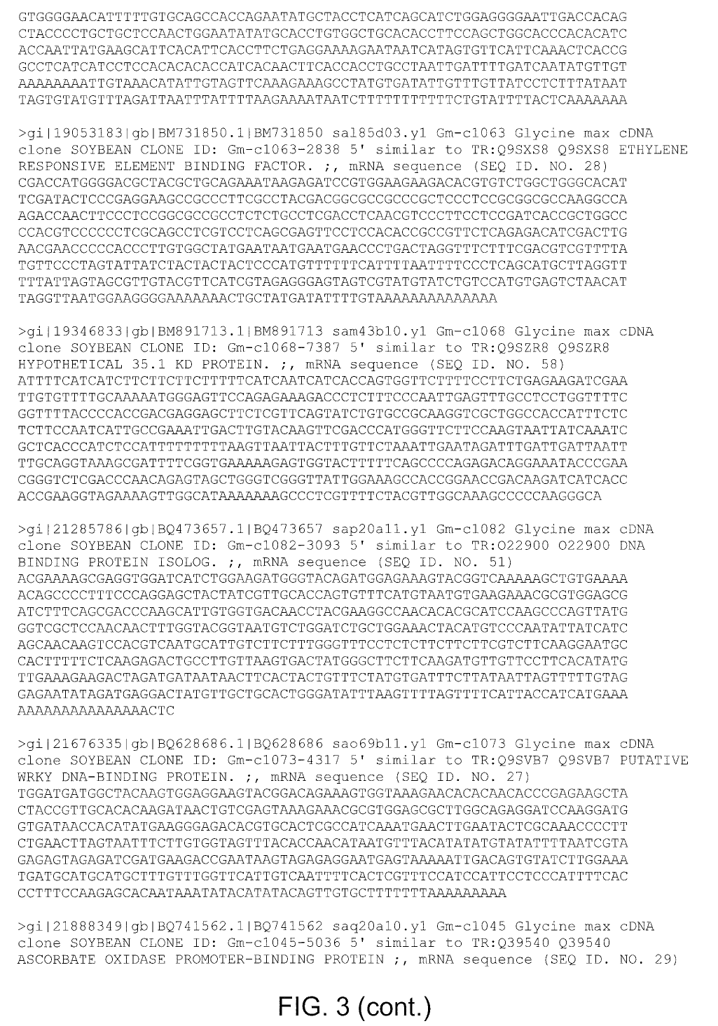

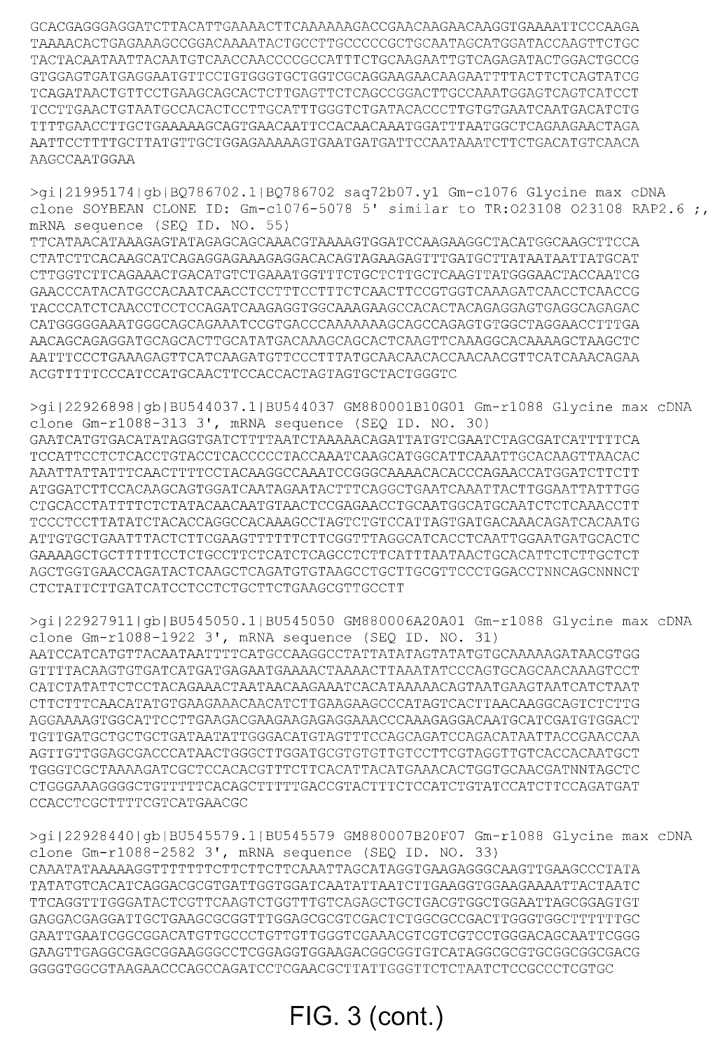

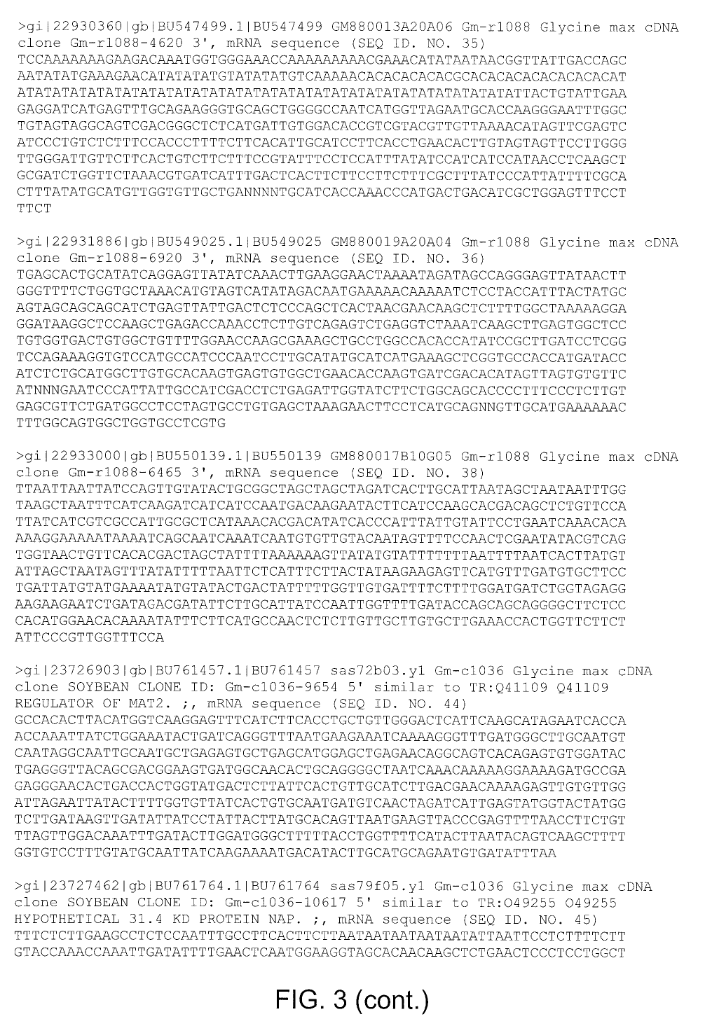

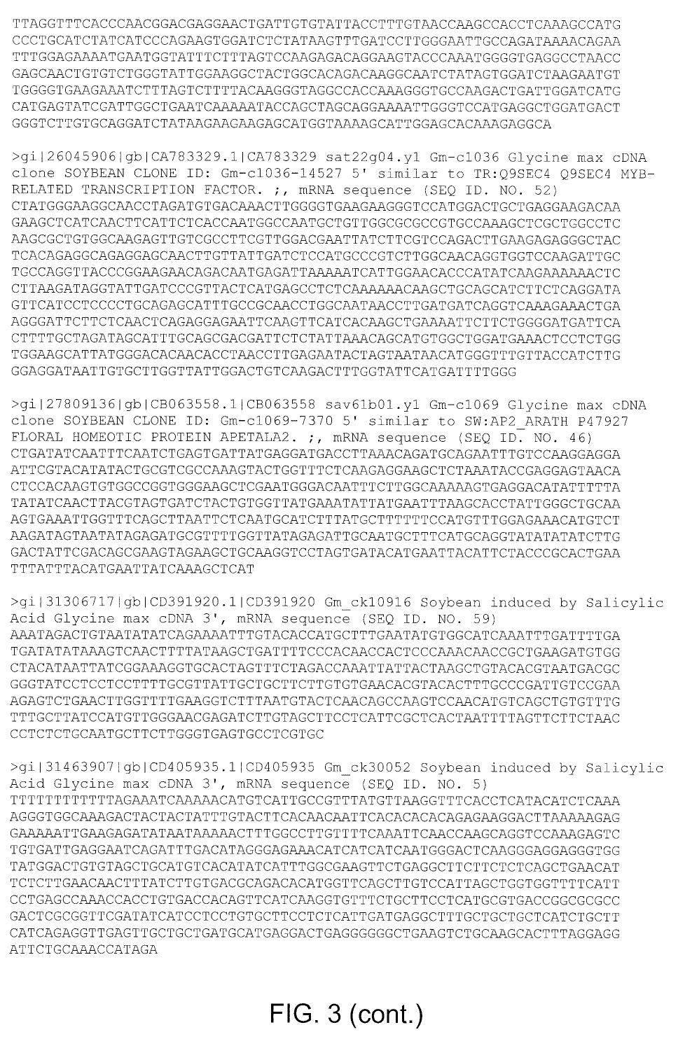

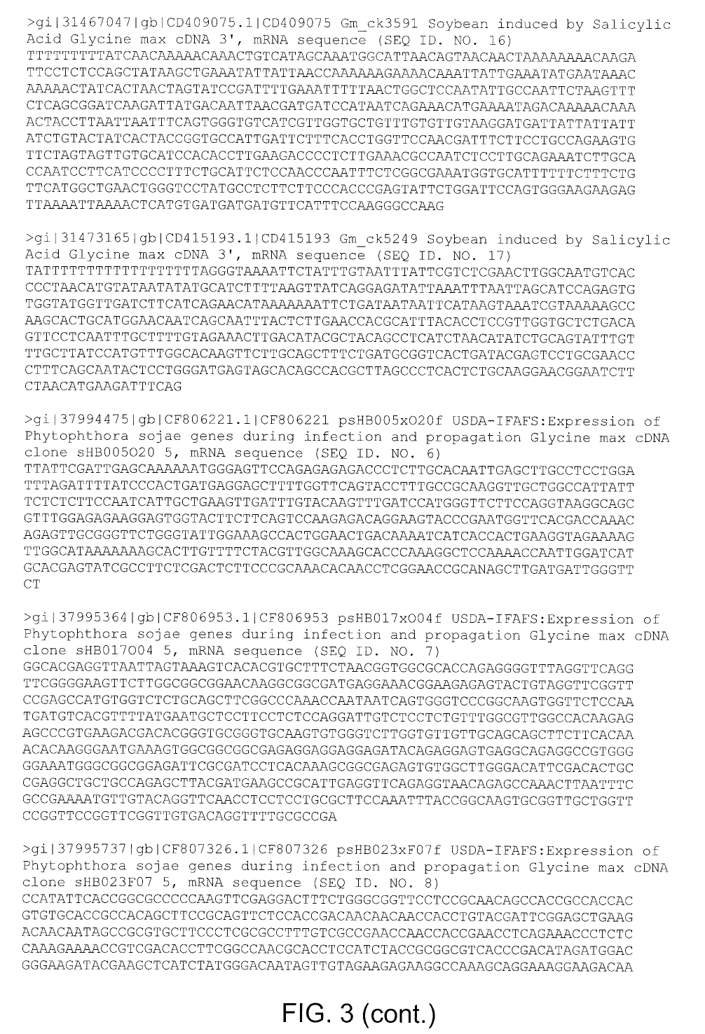

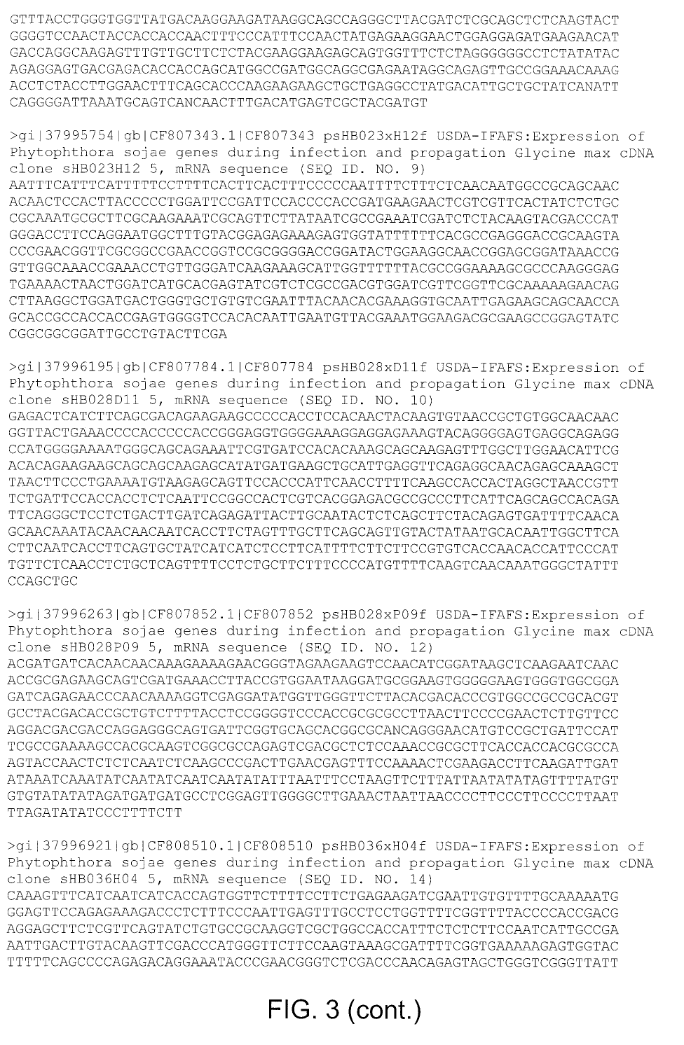

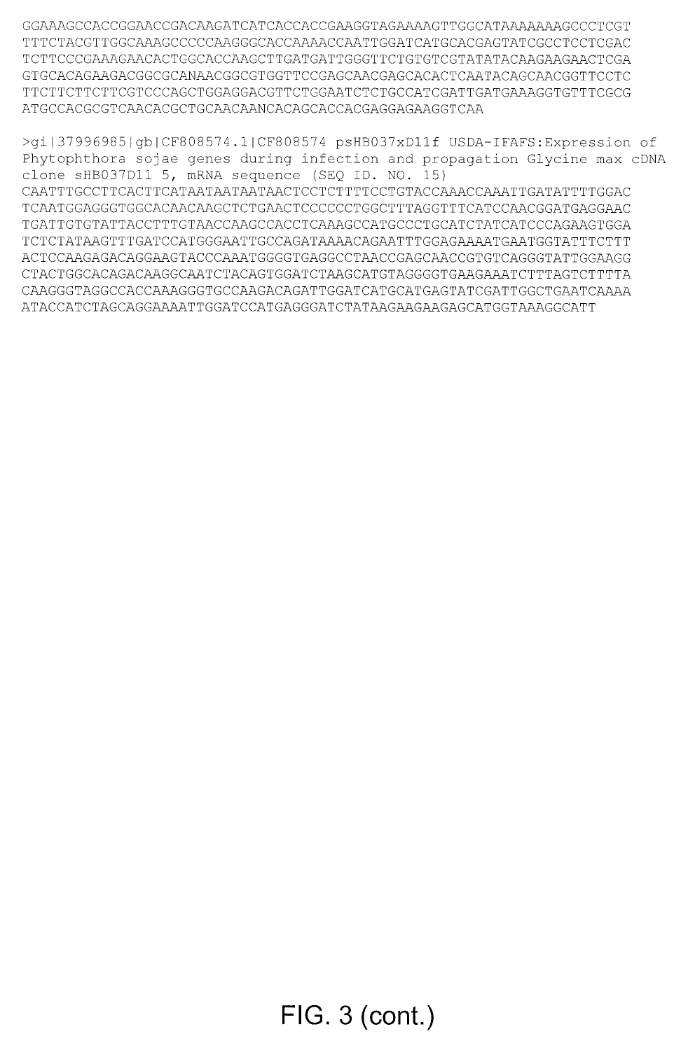

A set of 62 candidate drought response genes (or DRGs) identified in the microarray experiment were further confirmed by quantitative reverse transcription-PCR (qRT-RCR). Briefly, RNA samples from root or leaf tissues obtained from soybean plants grown under normal or drought conditions were prepared as described in Example 1. cDNA were prepared from these RNA samples by reverse transcription. The cDNA samples thus obtained were then used as template for PCR using primer pairs specific for 64 candidate genes. The PCR products of each gene under either drought or normal conditions were quantitated and the results are summarized in Table 5. The exact sequences of all transcripts listed in Table 5 are shown in FIG. 3. Some DRGs shown in Table 5 have been listed in Tables 2 or 4, while certain DRGs shown in Table 5 do not overlap with those listed in Tables 2 or 4. The Column with the heading “qRT-PCR Root log ratio of expression level” shows the base 2 logarithm of the ratio between the root expression level of the particular gene under drought condition and the expression level of the same gene under normal condition. Similarly, the Column with the heading “qRT-PCR Leaf log ratio of expression level” shows a similar set of data obtained from leaf tissues. The qRT-PCR results are generally consistent with the microarray data, suggesting that the genes whose expression levels are up-regulated or down-regulated are likely to be true Drought Response Genes (DRGs).

While the foregoing instrumentalities have been described in some detail for purposes of clarity and understanding, it will be clear to one skilled in the art from a reading of this disclosure that various changes in form and detail can be made without departing from the true scope of the invention. For example, all the techniques and apparatus described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

TABLE 5

qRT-PCR validation of Soybean transcripts expressed in the root microarray

| | NCBI | | | | qRT-PCR | qRT-PCR |
|---|---|---|---|---|---|---|
| | Acces- | | | | Root log | Leaf log |
| SEQ | sion# of | | | | ratio of | ratio of |
| ID | soybean | Fold Change | qRT-PCR Forward primer | qRT-PCR Reverse primer | expression | expression |
| No. | EST | in Microarray | (SEQ ID No.) | (SEQ ID No.) | level | level |
| 1 | AW100172 | 3.084026621 | ctgtgccagcgaactcaattaa | cttgcgagccctccttctc (66) | 1.179714697 | 0.895684575 |
| | | | (65) | | | |
| 2 | BI700189 | 5.250749017 | ttttcccggcatcacagatt (67) | gcggccatcggagattc (68) | 2.895301654 | 0.900519653 |
| 3 | AW101461 | 2.131337965 | tcatatgaggccatacaacaggaa | gttaggcagaggtggccaatt (70) | 3.218713127 | 1.099808494 |
| | | | (69) | | | |
| 4 | BI701724 | 2.445271745 | caaatgcgcttcgcaacata (71) | cccaagggtcgtacttgtacaga | 0.773064485 | 2.11599468 |
| | | | | (72) | | |
| 5 | CD405935 | 2.378775421 | cctcagaacttcgccaaatga (73) | agggaggagggtggtatgga (74) | 1.765969386 | 0.435720028 |
| 6 | CF806221 | 5.844540021 | cccaaaggctccaaaacca (75) | ggttgtgtttgcgggaagag (76) | 2.707173466 | 1.788682924 |
| 7 | CF806953 | 3.07486286 | ttccgagccatgtggtctct (77) | gccgggacccactgattat (78) | 2.428323563 | 31.96231869 |
| 8 | CF807326 | 2.533554706 | caacaatagccgcgtgctt (79) | tctgaggttcggtggttggt (80) | 4.313476213 | 0.869315235 |
| 9 | CF807343 | 8.420142043 | cgccgacgtggatcgt (81) | tcgacacagcacccagtcat (82) | 2.813139313 | 2.384971456 |
| 10 | CF807784 | 3.526862338 | tccacccattcaaccttttca (83) | ttgagaggtggtggaatcagaa | 0.751688585 | 5.961955746 |
| | | | | (84) | | |
| 11 | BE807836 | 11.39265251 | CACTATCGAGGACATAATAGACATTT | GGGCCAGGTGTGATTCATTAA (86) | 3.198592784 | 1.743447998 |
| | | | CAA (85) | | | |
| 12 | CF807852 | 3.418157687 | ttccccgaactcttgttcca (87) | cgtgctgcaccgaatcact (88) | 1.809994107 | 2.073651806 |
| 13 | AW507968 | 3.104335099 | atggcattgtggcaacttca (89) | tctgcaggcccgtgattatt (90) | 2.570471471 | 1.062284353 |
| 14 | CF808510 | 11.48486693 | GAAGAACTCGAGTGCACAGAAGAC | CTGTATTGAGTGTGCTCGTTGCT | 2.516019319 | 2.125569846 |
| | | | (91) | (92) | | |
| 15 | CF808574 | 6.774193077 | atggagggtggcacaacaag (93) | aatcagttcctcatccgttgga | 1.214925907 | 3.765955189 |
| | | | | (94) | | |
| 16 | CD409075 | 2.893022301 | aggacccagttcagccatga (95) | ctgcattctccaacccaatttc | 3.226927877 | 0.986515074 |
| | | | | (96) | | |
| 17 | CD415193 | 2.82518237 | gagcaccaacggaggtgtaaa (97) | ctgcatggaacaatcagcaatt | 1.600145026 | 1.402223191 |
| | | | | (98) | | |
| 18 | BE820446 | 2.634118248 | TTGGATATCCCACTTTTGCTTCA | ATCATGCGACATACTAACGCTACCT | 2.336783376 | 1.42179684 |
| | | | (99) | (100) | | |
| 19 | BE821438 | 2.543318408 | tcccaagcagtgcaacatagtc | ctcatgggtccgctgctatt (102) | 1.074857694 | 0.928756087 |
| | | | (101) | | | |
| 20 | BI321576 | 2.207357752 | cagaccccgtctactgcctact | gtggagggcccaatcatg (104) | 0.639898209 | 1.210508883 |
| | | | (103) | | | |
| 21 | BE821939 | 2.355222512 | tgcgctgacgtgtcatcac (105) | tcatgaactcgtccagatcgaa | 0.755689422 | 1.017449131 |
| | | | | (106) | | |
| 22 | BE822796 | 2.095832928 | aacctgatcatccgcacttga | ctgcagggaaagcaaaggttt | 2.064518481 | 0.574531142 |
| | | | (107) | (108) | | |
| 23 | BF324082 | 3.416959863 | aagtgacatccctaatctccatctta | agggaagtggcagtttggaa (110) | 2.936031948 | 0.112808916 |
| | | | a (109) | | | |
| 24 | BF325482 | 5.267479195 | tgttgcacatatgtctggatctacat | caggaagcccaaacattgga (112) | 2.842974187 | 1.262883887 |
| | | | (111) | | | |
| 25 | BF425742 | 2.068872398 | GGAACGACCAATTGTACATGGA | AGCTGGAGTCCGTGTGCAA (114) | 0.224027074 | 5.847374529 |
| | | | (113) | | | |
| 26 | BI427426 | 4.769527624 | GCACCTTTTGTCCTTGTAGGAAA | TCTCTTGATGCTGTTGAGGTAGCT | 0.826515435 | 0.635762723 |
| | | | (115) | (116) | | |
| 27 | BQ628686 | 4.497761581 | gcatgctttgtttggttcattg | tgctcttggaaagggtgaaaat | 2.562119323 | 0.992467433 |
| | | | (117) | (118) | | |
| 28 | BM731850 | 2.044991104 | ctcgtcctcagcgagttcct (119) | gggttcgttcaagtcgatgtc | 7.951057023 | 0 |
| | | | | (120) | | |
| 29 | BQ741562 | 10.24611681 | CTGAAGCAGCACTCTTGAGTTCTC | GCATTACAGTTCAAGGAAGGATGA | 15.99359845 | 1.697910013 |
| | | | (121) | (122) | | |
| 30 | BU544037 | 3.939302141 | ggctttgtggcctggtgtag (123) | cctgcaatggcatgcaatc (124) | 1.601244187 | 2.815531581 |
| 31 | BU545050 | 2.494897545 | gcattgtggtgacaacctacga | aaagttgttggagcgacccata | 1.329048728 | 2.107376374 |
| | | | (125) | (126) | | |
| 32 | BI945178 | 2.772128801 | tccgacaacctcaattcactgt | ggttggtacccgagagttgaaa | 0.92235029 | 11.83388602 |
| | | | (127) | (128) | | |
| 33 | BU545579 | 3.055064447 | aaccgcgcttcagcaatc (129) | tgctgacgtggctggaatta (130) | 0.628241724 | 1.590916738 |
| 34 | BE346777 | 2.151895139 | GTGGATTCACATGCTTCCAAAG | CACCACCAATTAACCCCATCA | 5.745522109 | 0.925283904 |
| | | | (130) | (131) | | |
| 35 | BU547499 | 5.270995487 | tcccttggtgcattctaacca | tgtattgaagaggatcatgagtttgc | 0.180701827 | 2.242966905 |
| | | | (132) | (133) | | |
| 36 | BU549025 | 5.875864511 | acacctttctggaccgaggat | ttttggaaccaagcgaaagc (135) | 4.889861716 | 0.645009508 |
| | | | (134) | | | |
| 37 | AW349551 | 2.153270217 | ctcgtcctcagcgagttcct (136) | tagttgcggcggttcaatg (137) | 0.704217833 | 2.973284126 |
| 38 | BU550139 | 3.139509682 | tcaggaatacaataaatgggtgatat | cgacagctctgttccattatcatc | 0.704949264 | 0.852237437 |
| | | | g (138) | (139) | | |
| 39 | AW351262 | 17.11708494 | TGTACTGGAACGTAATGGTGTGTGT | CAATTCCCATCCTCTCCAACA | 7.265947794 | 0.805102663 |
| | | | (140) | (141) | | |
| 40 | BG653183 | 2.017838456 | caacatctgcatcttctcagttgtc | tcaaaccccttgaggaagca (143) | 1.047227581 | 1.21660345 |
| | | | (142) | | | |
| 41 | AW458014 | 2.091595353 | ccccttttcacctgcaattct | tccattccccacaagctagtg | 3.60212605 | 0.965014595 |
| | | | (144) | (145) | | |
| 42 | BE658881 | 3.954686528 | tccgtgggtttgacctgaac (146) | ccatcggcgaccagaact (147) | 0.277411211 | 1.889361371 |
| 43 | AW459852 | 2.172823071 | cggaagcggctattggaa (148) | ccctagcgctttcggtttt (149) | 0.120999836 | 2.094198216 |
| 44 | BU761457 | 3.897946544 | aaagatgccgagagggaacac | gttcgtcaagatgcaacagtgaa | 18.4130026 | 1.271652661 |
| | | | (150) | (151) | | |
| 45 | BU761764 | 5.880074724 | TTAATTCCTCTTTTCTTGTACCAAAC | AAAGCCAGGAGGGAGTTCAGA | 1.170626898 | 1.602711404 |
| | | | C (152) | (153) | | |
| 46 | CB063558 | 2.30019111 | gcacctattgggctgcaaag (154) | gaaaaaagcataaagatgcattgaga | 5.600809401 | 2.040362749 |
| | | | | (155) | | |
| 47 | BI967585 | 2.27451735 | cgatgagcaggaccagatacc | cccaaaggagggaaagtaaggt | 1.70729339 | 0.506005159 |
| | | | (156) | (157) | | |
| 48 | BF070218 | 3.582174165 | aggcagtccaatttccaaagag | gcagggacaactcccaaca (159) | 2.614112084 | 1.511894704 |
| | | | (158) | | | |
| 49 | BI970890 | 2.476691576 | ctgatgccccctgaagaaatt | cacccaaacatgcagagatgag | 1.207628743 | 1.381055215 |
| | | | (160) | (161) | | |
| 50 | BI972938 | 3.803601179 | tgccgttaccatcaaacactaaac | gctaaccttggatgagaaagcaa | 1.623132753 | 1.350839558 |
| | | | (162) | (163) | | |
| 51 | BQ473657 | 3.265947707 | CTGGATCTGCTGGAAACTACATGT | GGAAACCCAAAGAAGACAATGC | 2.625389848 | 2.168943292 |
| | | | (164) | (165) | | |
| 52 | CA783329 | 3.61154719 | CAAAAAACAAGCTGCAGCATCT | CCAGGTTGCGGCAAATG (167) | 7.751069203 | 0.782186749 |
| | | | (166) | | | |
| 53 | BI784829 | 2.917788554 | aaagcatgcatcagcagcat (168) | tggttgtttgaggacagtgaaga | 5.493438033 | 0.74028789 |
| | | | | (169) | | |
| 54 | BI786091 | 4.256920675 | gcccccacctccacaact (170) | cacctcctggtgggttttca (171) | 0.558102237 | 14.04069068 |
| 55 | BQ786702 | 6.11243033 | gagcagcaaacgtaaaagtggat | gatgcttgtgaagatagtggaagct | 8.006220409 | 1.872437201 |
| | | | (172) | (173) | | |
| 56 | BM188078 | 5.347282485 | aactcaccggcctcatcatc (174) | aatcaattaggcaggtggtgaagt | 1.471782005 | 0.676653897 |
| | | | | (175) | | |
| 57 | BG790575 | 2.130840142 | cctcacagcagcaacacaatg | gctggttctgtaattgacctttttg | 16.3768237 | 0.592442208 |
| | | | (176) | (177) | | |
| 58 | BM891713 | 2.627768053 | tcagccccagagacaggaaa (178) | tagctgggtcgggttattgg (179) | 0 | 2.025252797 |
| 59 | CD391920 | 5.01907607 | tttccgataattatgtagccacatct | aagctgattttcccacaacca | 9.769844952 | 1.694022457 |
| | | | (180) | (181) | | |
| 60 | BI893143 | 2.349057984 | ttgtcgcatgcccagaatc (182) | ttggcaatatttgtgatgcatgt | 0 | 0 |
| | | | | (183) | | |
| 61 | BM094926 | 2.10592882 | tcaaaggttcccagccaaac (184) | tgggcatcggagattcga (185) | 0.376159561 | 0.907837298 |
| 62 | BM094932 | 2.04661982 | aagaccaaagggtgctcaacat | ttccaaagtgcccagatgaag | 1.662781575 | 1.520080787 |
| | | | (186) | (187) | | |
| 63 | D26092 | Endo control | AGCTATTCGCAGTTCCCAAAT | CAGAGACGAACCTTGAGGAGA | 1 | 1 |
| | | | (188) | (189) | | |
| 64 | J01298 | Endo control | CGTTAACTTTTCCCCTTCGCTC | CGAGGACGACCAACAATGCTA | 1.29685184 | 0.499685295 |
| | | | (190) | (191) | | |

### REFERENCES

In addition to those references that are cited in full in the text, additional information for those abbreviated citations is listed below:
Boyer, J S, 1983, Environmental stress and crop yields. In C. D. Raper and P. J. Kramer (ed) Crop reactions to water and temperature stresses In humid, temperature climates. Westview press, Boulder, Colo. pp 3-7.
Muchow R C, Sinclair T R. 1988. Water and nitrogen limitations In soybean grain production. II. Field and model analyses. Field Crop Res. 15:143-158.
Specht J E, Hume D J, Kumind S V. 1999. Soybean yield potential-A genetic physiological perspective. Crop Science 39:1560-1570.
Wang W, Vinocur B, Altman A: Plant responses to drought, salinity and extreme temperatures: towards genetic engineering for stress tolerance. Planta 2003, 218:1-14.
Vinocur, B, Altman A: Recent advances in engineering plant tolerance to abiotic stress: achievements and limitations. Curr Opin Biotech 2005, 16:123-32.
Chaves M M, Oliveire M M: Mechanisms underlying plant resilience to water deficits: prospects for water-saving agriculture. J Exp Bot 2004, 55; 2365-2384.
Shinozaki K, Yamaguchi-Shinozaki K, Seki M: Regulatory network of gene expression in the drought and cold stress responses. Curr Opin Plant Biol 2003, 6:410-417.
Schena M, Shalon D, Davis R W, Brown P O (1995) Quantitative monitoring of gene expression patterns with a complementary DNA microarray. Science 270: 467-470
Shalon D, Smith S, Brown P (1990) A DNA microarray system for analyzing complex DNA samples using two-color fluorescent probe hybridization. Genome Res. 8: 639-645.
Bray E A: Genes commonly regulated by water-deficit stress in Arabidopsis thaliana. J Exp Bot 2004, 55:2331-2341.
Denby K, Gehring C: Engineering drought and salinity tolerance in plants: lessons from genome-wide expression profiling In Arabidopsis. Trends in Plant Sci 2005, 23547-552.
Shinozaki K, Yamaguchi-Shinozaki K: Molecular responses to drought and cold stress. Curr Opin Biotech 1996, 7:181-167
Shinozaki. K. and Yamaguchi-Shinozaki, K: Molecular responses to dehydration and low temperature; differences and cross-talk between two stress signaling pathways. Curr Opin Plant Biol 2000, 3:217-223.
Seki M, Narusaka M, Abe H, Kasuga M, Yamaguchi-Shinozaki K, Carninci P, Hayashizaki Y, Shinozaki K: Monitoring the expression pattern of 1300 Arabidopsis genes under drought and cold stresses by using a full-length cDNA microarray. Plant Cell 2001, 13:61-72.
Fowler S, Thomashow M F: Arabidopsis transcriptome profiling indicates that multiple regulatory pathways are activated during cold acclimation In addition to the CBF cold response pathway, Plant Cell 2002, 14:1875-1690.
Maruyama K, Sakuma Y, Kasuga M, Ito Y, Seki M, Goda H, Shimada Y, Yoshida S, Shinozaki K, Yamaguchi-Shinozaki K: Identification of cold-inducible downstream genes of the Arabidopsis DREB1A/CBF3 transcriptional factor using two microarray systems. Plant J 2004, 38:982-993.

We claim:

1. A method for generating a transgenic plant from a host plant, said transgenic plant being more tolerant to drought condition when compared to the host plant, said method comprising a step of altering the expression levels of a protein encoded by a Drought Response Gene (DRG) or a fragment thereof, said Drought Response Gene being endogenous to the host plant.

2. The method of claim 1, wherein the expression levels of the protein are altered by modifying the transcription regulation of the Drought Response Gene.

3. A method for generating a transgenic plant from a host plant, said transgenic plant being more tolerant to drought condition when compared to the host plant, said method comprising the steps of:
(a) introducing into a plant cell a construct comprising a Drought Response Gene (DRG) or a fragment thereof encoding a polypeptide; and
(b) generating a transgenic plant expressing said polypeptide or a fragment thereof.

4. The method of claim 3, wherein said Drought Response Gene or a fragment thereof is derived from a plant that is genetically different from the host plant.

5. The method of claim 3, wherein said Drought Response Gene or a fragment thereof is derived from a plant that belongs to the same species as the host plant.

6. The method of claim 3, wherein the Drought Response Gene or a fragment thereof comprises a sequence selected from the group consisting of the sequences from SEQ ID. No. 1 to SEQ ID. No. 62.

7. The method of claim 3, wherein the coding sequence of said Drought Response Gene or a fragment thereof is operably linked to a promoter for regulating expression of said polypeptide.

8. The method of claim 7, wherein the promoter is derived from another gene that is different from said Drought Response Gene.

9. A method for generating a transgenic plant from a host plant, said transgenic plant being more tolerant to drought condition when compared to the host plant, said method comprising the steps of:
(a) introducing into a plant cell a construct comprising a DNA sequence encoding a first polypeptide that is at least 90% identical to a second polypeptide encoded by a Drought Response Gene (DRG), said Drought Response Gene being derived from a source selected from the group consisting of a plant that is genetically different from the host plant; and
(b) generating a transgenic plant expressing said first polypeptide or a fragment thereof.

10. The method of claim 9, wherein the Drought Response Gene comprises a sequence selected from the group consisting of the sequences from SEQ ID. No. 1 to SEQ ID. No. 62.

11. The method of claim 9, wherein the coding region of said DNA sequence is operably linked to a promoter for regulating expression of said first polypeptide or a fragment thereof.

12. The method of claim 11, wherein the promoter is at least one member selected from the group consisting of a cell-specific promoter, a tissue specific promoter, an organ specific promoter, a constitutive promoter, and an inducible promoter.

13. The method according to claim 11, wherein at least a portion of said DNA sequence is oriented in an antisense direction relative to said promoter within said construct.

14. The method of claim 9, wherein the first polypeptide is at least 98% identical to the second polypeptide encoded by said Drought Response Gene.

15. The method of claim 9, wherein the first polypeptide is at least 99% identical to the second polypeptide encoded by said Drought Response Gene.

16. A method for generating a transgenic plant from a host plant, said transgenic plant being more tolerant to drought condition when compared to the host plant, said method comprising the steps of:
(a) introducing into a plant cell a construct comprising a DNA sequence selected from the group consisting of a soybean Drought Response Gene or a fragment thereof, and a homolog of a soybean Drought Response Gene or a fragment thereof; and
(b) generating a transgenic plant expressing a polypeptide encoded by said DNA sequence.

17. The method of claim 16, wherein the homolog is derived from a plant other than soybean, said homolog encoding a first polypeptide that is at least 90% identical to a second polypeptide encoded by the soybean Drought Response Gene.

18. The method of claim 16, wherein the host plant is selected from the group consisting of soybean, corn, wheat, rice, and cotton.

19. A method for generating a transgenic plant from a host plant, said transgenic plant being more tolerant to drought condition when compared to the host plant, said method comprising the steps of:
(a) introducing into a plant cell a construct comprising at least one DNA sequence, said at least one DNA sequence being derived from a soybean Drought Response Gene or its homolog from a plant that is genetically different from soybean, said soybean Drought Response Gene comprising a sequence selected from the group consisting of sequences from SEQ ID. No. 1 to SEQ ID. No. 62; and
(b) generating a transgenic plant expressing a polypeptide encoded by said DNA sequence.

20. A transgenic plant generated from a host plant using the method of claim 1, claim 3, claim 9, claim 16 or claim 19, said transgenic plant exhibiting increased tolerance to drought as compared to the host plant.

21. A transgenic plant generated from a host plant, said transgenic plant comprising a transgene, wherein the transgene comprises a Drought Response Gene (DRG) or a fragment thereof, and said Drought Response Gene (DRG) or a fragment thereof is derived from a plant that is genetically different from the host plant.

22. The transgenic plant of claim 21, wherein the transgene comprises a sequence selected from the group consisting of sequences from SEQ ID. No. 1 to SEQ ID. No. 62.

23. The transgenic plant of claim 21, wherein the coding regions of the transgene is operably linked to a promoter for regulating expression of said transgene.

24. The transgenic plant of claim 23, wherein the promoter is at least one member selected from the group consisting of a cell-specific promoter, a tissue specific promoter, an organ specific promoter, a constitutive promoter, and an inducible promoter.

25. A transgenic plant generated from a host plant, said transgenic plant comprising a Drought Response Gene (DRG) or a fragment thereof encoding a polypeptide, wherein the Drought Response Gene is derived from a plant that belongs to the same species as the host plant, and the expression levels the polypeptide encoded by said Drought Response Gene is altered such that the expression levels of said polypeptide in the transgenic plant is at least 50% higher or lower than the expression levels of said polypeptide in the host plant.

26. The transgenic plant of claim 25, wherein the host plant is selected from the group consisting of soybean, corn, wheat, rice, and cotton.
